(19) United States
(12) Patent Application Publication
(10) Pub. No.: US 2026/0209740 A1
(43) Pub. Date: Jul. 23, 2026

(54) LIGASE AND DEHYDROGENASE VARIANTS AND METHODS OF USE

(71) Applicant: GENOMATICA, INC., San Diego, CA (US)

(72) Inventors: Bob VAN HOVE, Dedham, MA (US); Jonathan Lewis MCMURRY, Boston, MA (US); Dylan Alexander CARLIN, Sonoma, CA (US); Fiona QU, Cambridge, MA (US); Ana Elisa MARTINEZ DEL CAMPO, Somerville, MA (US); Amit Mahendra SHAH, San Diego, CA (US); Harish NAGARAJAN, San Diego, CA (US); Anke René R. GOORMANS, Boston, MA (US)

(21) Appl. No.: 19/144,382

(22) PCT Filed: Dec. 28, 2023
(86) PCT No.: PCT/US2023/086280

§ 371 Date: Jun. 27, 2025

Related U.S. Application Data
(60) Provisional application No. 63/478,069, filed on Dec. 30, 2022.

(51) Int. Cl. C12N 9/00; C12N 1/20; C12N 9/02; C12P 7/18; C12P 7/24; C12P 19/32

(52) U.S. Cl. CPC C12N 9/93; C12N 1/20; C12N 9/0008; C12P 7/18; C12P 7/24; C12P 19/32; C12Y 602/01003

(57) ABSTRACT

The disclosure provides polypeptides and encoding nucleic acids of engineered acyl coenzyme-A ligases, engineered In acetylating aldehyde dehydrogenases or both. The disclosure also provides cells expressing an engineered form of the acyl coenzyme-A ligases, acetylating aldehyde dehydrogenases or both. The disclosure further provides methods for producing a bioderived compound, such as 6-hydroxyhexanoyl coenzyme-A, 6-hydroxy hexanal, 1,6 hexanediol, 6-aminocaproyl-CoA, 6-aminocaproate semialdehyde, hexamethylenediamine, and products derived therefrom comprising culturing cells expressing an engineered acyl coenzyme-A ligases, engineered acetylating aldehyde dehydrogenases or both.

### CROSS-REFERENCE

This application claims benefit of U.S. Provisional Application No. 63/478,069, filed on Dec. 30, 2022, which is incorporated herein by reference in its entirety.

### INCORPORATION BY REFERENCE OF SEQUENCE LISTING

The instant application contains a Sequence Listing, which has been submitted via Patent Center. The Sequence Listing titled 199683-141001_PCT_SL.xml, which was created on Oct. 31, 2023 and is 104,371 bytes in size, is hereby incorporated by reference in its entirety.

### FIELD

The present disclosure relates generally to acyl coenzyme-A ligase variants, acetylating aldehyde dehydrogenase variants, or both and methods of using such enzymes, and more specifically to acyl coenzyme-A ligase variants, acetylating aldehyde dehydrogenase variants, or both encoded by recombinant nucleic acids that have been introduced to a non-naturally occurring microbial organism to produce a bioderived compound such as 6-hydroxyhexanoyl coenzyme-A, 6-hydroxyhexanal, 1,6 hexanediol, 6-aminocaproyl-CoA, 6-aminocaproate semialdehyde, hexamethylenediamine, and products derived therefrom.

### BACKGROUND OF THE INVENTION

Various commodity chemicals are used to make desired products for commercial use. Many of the commodity chemicals are derived from petroleum. Such commodity chemicals have various uses, including use as solvents, resins, polymer precursors, and specialty chemicals. Desired commodity chemicals include 6-carbon molecules, such as, 1,6 hexanediol (HDO), and hexamethylenediamine (HMD), upstream precursors (e.g., 6-hydroxyhexanoyl coenzyme-A, 6-hydroxyhexanal, 6-aminocaproyl-CoA, 6-aminocaproate semialdehyde and caprolactam) and downstream products. HDO is an intermediate in the production of HMD and nylon 6,6. (O'Neil, M. J. (ed.). The Merck Index−An Encyclopedia of Chemicals, Drugs, and Biologicals. Cambridge, UK: Royal Society of Chemistry, 2013., p. 868). Nylons are polyamides that can be synthesized by the condensation polymerization of a diamine with a dicarboxylic acid or the condensation polymerization of lactams. Nylon 6,6 can also be produced by reaction of HMD and adipic acid, while nylon 6 is produced by a ring opening polymerization of caprolactam. Therefore, HDO, HMD and upstream precursors are commodity chemicals.

Microorganisms have been engineered to produce some of the nylon intermediates. However, engineered microorganisms can produce undesirable byproducts as a result of undesired enzymatic activity on pathway intermediates and final products. Such byproducts and impurities therefore increase cost and complexity of biosynthesizing compounds and can decrease efficiency or yield of the desired products.

### SUMMARY OF INVENTION

In some embodiments, provided herein is an engineered acyl-coenzyme A ligase (ACL) that is a variant of amino acid sequence SEQ ID NO: 1 or a functional fragment thereof, wherein the engineered ACL comprises one or more alterations at a position described in TABLE 8; or is a variant of amino acid sequence SEQ ID NO: 2 or a functional fragment thereof, wherein the engineered ACL comprises one or more alterations at a position described in TABLE 9. An engineered ACL described herein, in some embodiments, is capable of: (a) catalyzing the conversion of 6-hydroxyhexanoate (HHA) to 6-hydroxyhexanoyl coenzyme A (HHA-CoA); (b) catalyzing the conversion of HHA to HHA-CoA at a greater rate compared to wild-type ACL; (c) having a higher specificity for the HHA substrate as compared to the wild type ACL; (d) catalyzing the conversion of 6-aminocaproate (6ACA) to 6-aminocaproyl-CoA (6ACA-CoA); (e) catalyzing the conversion of 6ACA to 6ACA-CoA at a greater rate compared to wild-type ACL; (f) having a higher specificity for the 6ACA substrate as compared to the wild type ACL or (g) any combination thereof. In some embodiments, an engineered ACL described herein has higher specificity for conversion of HHA to HHA-CoA, or 6ACA to 6ACA-CoA, or both over conversion of: (a) propionate to propionyl-CoA; (b) acetate to acetyl-CoA; (c) adipate to adipyl-CoA; (d) succinate to succinyl-CoA; (e) 4-hydroxybutyrate (4-HB) to 4-hydroxybutyryl-CoA (4-HBC); (f) 3-hydroxybutyrate (3HB) to 3-hydroxybutyrate-CoA (3HB-CoA); (g) 3-hydroxybutyrate (3HB) to either isomer of 3-hydroxybutyrate-CoA (3HB-CoA); or (h) any combination thereof. An engineered ACL described herein, in some embodiments, has higher specificity for conversion of adipate to adipyl-CoA, succinate to succinyl-CoA, or both, over conversion of 4-HB to 4-HBC.

In some embodiments, an engineered ACL described herein has an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an ACL consisting of the amino acid sequence of SEQ ID NO: 1 or SEQ ID NO: 2.

In some embodiments, an engineered ACL described herein having one or more amino acid alterations comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 or more alterations. An engineered ACL described herein, in some embodiments, includes one or more amino acid alterations that are conservative or non-conservative amino acid substitutions, deletions, or insertions, or any combination thereof.

In some embodiments, an engineered ACL described herein includes one or more amino acid alterations at a position corresponding to position 4, 7, 8, 9, 10, 12, 13, 15, 16, 19, 20, 22, 30, 31, 35, 36, 39, 40, 43, 44, 47, 48, 49, 50, 51, 52, 53, 54, 55, 60, 61, 64, 66, 67, 69, 72, 73, 74, 76, 78, 80, 81, 82, 83, 86, 87, 88, 89, 90, 91, 94, 95, 96, 97, 98, 99, 100, 101, 102, 103, 104, 105, 106, 107, 109, 111, 112, 113, 114, 115, 116, 117, 119, 121, 126, 127, 128, 129, 130, 131, 136, 137, 138, 139, 140, 144, 149, 150, 152, 153, 159, 162, 163, 164, 165, 166, 167, 170, 172, 182, 189, 191, 194, 195, 196, 197, 198, 199, 200, 201, 202, 205, 208, 209, 211, 212, 213, 214, 215, 217, 220, 221, 222, 223, 224, 225, 226, 227, 228, 229, 230, 231, 232, 233, 234, 235, 238, 239, 240, 241, 242, 243, 244, 246, 247, 248, 249, 250, 251, 252, 253, 254, 255, 256, 257, 258, 259, 260, 261, 262, 264, 265, 266, 267, 268, 269, 270, 271, 272, 274, 275, 276, 277, 278, 279, 280, 281, 282, 286, 287, 288, 289, 290, 291, 294, 295, 296, 297, 298, 299, 300, 304, 306, 309, 310, 311, 314, 315, 316, 317, 318, 319, 320, 321, 323, 324, 326, 327, 328, 329, 330, 331, 334, 335, 336, 338, 339, 341, 342, 348, 351, 352, 353, 354, 355, 356, 357, 359, 360, 362, 363, 364, 365, 366, 367, 368, 369, 370, 371, 372, 373, 374, 375, 376, 377, 378, 379, 380, 381, 383, 384, 386, 387, 388, 389, 390, 391, 394, 398, 402, 406, 407, 410, 411, 412, 413, 414, 415, 416, 418, 419, 420, 424, 425, 426, 430, 431, 432, 433, 436, 442, 443, 444, 445, 446, 447, 455, 459, 461, 462, 464, 465, 469, 470, 471, 472, 474, 475, 476, 477, 478, 479, 480, 481, 484, 486, 489, 490, 491, 493, 494, 496, 503, 506, 507, 508, 509, 513, 518, 519, 521, 523, 524, 525, 527, 528, 529, 539, 540, 542, 546, 551, 553, 554, 555, 557, 559, 560, 562, 564, 565, 566, 583, or a combination thereof, in SEQ ID NO: 1.

In some embodiments, an engineered ACL described herein includes one or more amino acid alterations at a position corresponding to position 7, 8, 10, 12, 15, 16, 19, 52, 54, 64, 66, 67, 73, 74, 82, 87, 90, 94, 100, 111, 115, 117, 119, 121, 129, 131, 138, 139, 140, 144, 150, 165, 166, 172, 182, 191, 199, 209, 212, 217, 221, 223, 224, 227, 229, 230, 231, 232, 233, 234, 235, 238, 240, 243, 249, 253, 254, 256, 258, 259, 262, 268, 271, 275, 277, 281, 288, 290, 296, 297, 300, 304, 306, 316, 318, 320, 321, 323, 328, 330, 331, 336, 352, 353, 363, 364, 365, 366, 369, 372, 374, 376, 378, 388, 390, 394, 398, 406, 407, 416, 432, 443, 445, 455, 462, 469, 470, 471, 480, 481, 484, 486, 490, 491, 493, 494, 496, 508, 513, 518, 519, 521, 523, 525, 546, 554, 555, 560, 565, 583 or a combination thereof, in SEQ ID NO: 1.

In some embodiments, an engineered ACL includes one or more amino acid alterations at a position corresponding to position 10, 199, 223, 229, 230, 231, 232, 233, 234, 235, 238, 243, 256, 258, 277, 288, 300, 318, 320, 321, 323, 328, 330, 364, 365, 369, 372, 374, 378, 462, 469, 480, 481, 484, 513, 523, 525, 554, 560, 583 or any combination thereof, in SEQ ID NO: 1.

In some embodiments, an engineered ACL includes one or more amino acid alterations at a position corresponding to position 223, 232, 233, 234, 238, 258, 288, 318, 320, 321, 323, 364, 513, 523 or any combination thereof, in SEQ ID NO: 1.

In some embodiments, an engineered ACL described herein having one or more amino acid alterations results in an engineered ACL including: (a) M at a residue corresponding to position 4 in SEQ ID NO: 1; (b) D, K, Q, S, T at a residue corresponding to position 7 in SEQ ID NO: 1; (c) I, S at a residue corresponding to position 8 in SEQ ID NO: 1; (d) A, K, S at a residue corresponding to position 9 in SEQ ID NO: 1; (e) F, H, P, V, W, Y at a residue corresponding to position 10 in SEQ ID NO: 1; (f) D, E, H, P at a residue corresponding to position 12 in SEQ ID NO: 1; (g) C, M at a residue corresponding to position 13 in SEQ ID NO: 1; (h) D, I, L, P at a residue corresponding to position 15 in SEQ ID NO: 1; (i) E, H, N at a residue corresponding to position 16 in SEQ ID NO: 1; (j) F, L, P, Q at a residue corresponding to position 19 in SEQ ID NO: 1; (k) W at a residue corresponding to position 20 in SEQ ID NO: 1; (1) S at a residue corresponding to position 22 in SEQ ID NO: 1; (m) D at a residue corresponding to position 30 in SEQ ID NO: 1; (n) A at a residue corresponding to position 31 in SEQ ID NO: 1; (o) G, P at a residue corresponding to position 35 in SEQ ID NO: 1; (p) H at a residue corresponding to position 36 in SEQ ID NO: 1; (q) A at a residue corresponding to position 39 in SEQ ID NO: 1; (r) C, F, L, M at a residue corresponding to position 40 in SEQ ID NO: 1; (s) G at a residue corresponding to position 43 in SEQ ID NO: 1; (t) D, H, S, T at a residue corresponding to position 44 in SEQ ID NO: 1; (u) R, RTYTQKEL (SEQ ID NO: 15), RTYTQREL (SEQ ID NO: 16), RTYT (SEQ ID NO: 17) at a residue corresponding to position 47 in SEQ ID NO: 1; (v) a deletion at a residue corresponding to position 48 in SEQ ID NO: 1; (w) a deletion, Y at a residue corresponding to position 49 in SEQ ID NO: 1; (x) a deletion, T at a residue corresponding to position 50 in SEQ ID NO: 1; (y) a deletion, F, H, L, R, W, Y at a residue corresponding to position 51 in SEQ ID NO: 1; (z) a deletion, K, R at a residue corresponding to position 52 in SEQ ID NO: 1; (aa) a deletion at a residue corresponding to position 53 in SEQ ID NO: 1; (bb) a deletion, L at a residue corresponding to position 54 in SEQ ID NO: 1; (cc) L at a residue corresponding to position 55 in SEQ ID NO: 1; (dd) R at a residue corresponding to position 60 in SEQ ID NO: 1; (ee) F, L at a residue corresponding to position 61 in SEQ ID NO: 1; (ff) A, G at a residue corresponding to position 64 in SEQ ID NO: 1; (gg) R at a residue corresponding to position 66 in SEQ ID NO: 1; (hh) A at a residue corresponding to position 67 in SEQ ID NO: 1; (ii) L at a residue corresponding to position 69 in SEQ ID NO: 1; (jj) G, T at a residue corresponding to position 72 in SEQ ID NO: 1; (kk) F, G, L, V at a residue corresponding to position 73 in SEQ ID NO: 1; (ll) K at a residue corresponding to position 74 in SEQ ID NO: 1; (mm) T at a residue corresponding to position 76 in SEQ ID NO: 1; (nn) V at a residue corresponding to position 78 in SEQ ID NO: 1; (oo) G at a residue corresponding to position 80 in SEQ ID NO: 1; (pp) PG at a residue corresponding to position 81 in SEQ ID NO: 1; (qq) A, E, G, N, R, T at a residue corresponding to position 82 in SEQ ID NO: 1; (rr) a deletion, K, P, R, S at a residue corresponding to position 83 in SEQ ID NO: 1; (ss) I at a residue corresponding to position 86 in SEQ ID NO: 1; (tt) I, V, VAV at a residue corresponding to position 87 in SEQ ID NO: 1; (uu) a deletion, A at a residue corresponding to position 88 in SEQ ID NO: 1; (vv) a deletion, I, L, V at a residue corresponding to position 89 in SEQ ID NO: 1; (ww) C, L, M, V, W at a residue corresponding to position 90 in SEQ ID NO: 1; (xx) A, L, M, S at a residue corresponding to position 91 in SEQ ID NO: 1; (yy) C, CIEAA (SEQ ID NO: 18), S, T, TIEAA (SEQ ID NO: 19) at a residue corresponding to position 94 in SEQ ID NO: 1; (zz) a deletion at a residue corresponding to position 95 in SEQ ID NO: 1; (aaa) a deletion, E at a residue corresponding to position 96 in SEQ ID NO: 1; (bbb) a deletion at a residue corresponding to position 97 in SEQ ID NO: 1; (ccc) a deletion, A, L at a residue corresponding to position 98 in SEQ ID NO: 1; (ddd) C, M at a residue corresponding to position 99 in SEQ ID NO: 1; (eee) S, M, L at a residue corresponding to position 100 in SEQ ID NO: 1; (fff) F, M at a residue corresponding to position 101 in SEQ ID NO: 1; (ggg) H, F, L at a residue corresponding to position 102 in SEQ ID NO: 1; (hhh) G at a residue corresponding to position 103 in SEQ ID NO: 1; (iii) V, L, T at a residue corresponding to position 104 in SEQ ID NO: 1; (jjj) V, W, H, A, I at a residue corresponding to position 105 in SEQ ID NO: 1; (kkk) S, Y, R, K at a residue corresponding to position 106 in SEQ ID NO: 1; (lll) A, C, V at a residue corresponding to position 107 in SEQ ID NO: 1; (mmm) L, V, Q at a residue corresponding to position 109 in SEQ ID NO: 1; (nnn) V, A, M, L, C, VTPAN (SEQ ID NO: 20) at a residue corresponding to position 111 in SEQ ID NO: 1; (ooo) T, a deletion at a residue corresponding to position 112 in SEQ ID NO: 1; (ppp) a deletion, L at a residue corresponding to position 113 in SEQ ID NO: 1; (qqq) a deletion, A, L, V at a residue corresponding to position 114 in SEQ ID NO: 1; (rrr) a deletion, N at a residue corresponding to position 115 in SEQ ID NO: 1; (sss) A, W, P at a residue corresponding to position 116 in SEQ ID NO: 1; (ttt) S at a residue corresponding to position 117 in SEQ ID NO: 1; (uuu) T at a residue corresponding to position 119 in SEQ ID NO: 1; (vvv) R, D at a residue corresponding to position 121 in SEQ ID NO: 1; (www) H at a residue corresponding to position 126 in SEQ ID NO: 1; (xxx) M, L, MRHTG (SEQ ID NO: 21), MRITG (SEQ ID NO: 22) at a residue corresponding to position 127 in SEQ ID NO: 1; (yyy) a deletion at a residue corresponding to position 128 in SEQ ID NO: 1; (zzz) a deletion, I, H at a residue corresponding to position 129 in SEQ ID NO: 1; (aaaa) a deletion at a residue corresponding to position 130 in SEQ ID NO: 1; (bbbb) a deletion, G, D at a residue corresponding to position 131 in SEQ ID NO: 1; (cccc) L at a residue corresponding to position 136 in SEQ ID NO: 1; (dddd) T at a residue corresponding to position 137 in SEQ ID NO: 1; (eeee) D, S at a residue corresponding to position 138 in SEQ ID NO: 1; (ffff) E at a residue corresponding to position 139 in SEQ ID NO: 1; (gggg) E, K, D at a residue corresponding to position 140 in SEQ ID NO: 1; (hhhh) T at a residue corresponding to position 144 in SEQ ID NO: 1; (iiii) R at a residue corresponding to position 149 in SEQ ID NO: 1; (jjjj) I, L at a residue corresponding to position 150 in SEQ ID NO: 1; (kkkk) A, T, C, I at a residue corresponding to position 152 in SEQ ID NO: 1; (llll) G, S at a residue corresponding to position 153 in SEQ ID NO: 1; (mmmm) G at a residue corresponding to position 159 in SEQ ID NO: 1; (nnnn) S, H, R, SIDAF (SEQ ID NO: 23), RIESF (SEQ ID NO: 24), HFDSF (SEQ ID NO: 25), HFDAF (SEQ ID NO: 26), HIDSF (SEQ ID NO: 27) at a residue corresponding to position 162 in SEQ ID NO: 1; (oooo) F, I, a deletion at a residue corresponding to position 163 in SEQ ID NO: 1; (pppp) a deletion, D at a residue corresponding to position 164 in SEQ ID NO: 1; (qqqq) a deletion, S at a residue corresponding to position 165 in SEQ ID NO: 1; (rrrr) a deletion, F at a residue corresponding to position 166 in SEQ ID NO: 1; (ssss) L at a residue corresponding to position 167 in SEQ ID NO: 1; (tttt) G at a residue corresponding to position 170 in SEQ ID NO: 1; (uuuu) L, A, D at a residue corresponding to position 172 in SEQ ID NO: 1; (vvvv) Q at a residue corresponding to position 182 in SEQ ID NO: 1; (wwww) E, G at a residue corresponding to position 189 in SEQ ID NO: 1; (xxxx) A at a residue corresponding to position 191 in SEQ ID NO: 1; (yyyy) D at a residue corresponding to position 194 in SEQ ID NO: 1; (zzzz) T, P, M at a residue corresponding to position 195 in SEQ ID NO: 1; (aaaaa) T, I at a residue corresponding to position 196 in SEQ ID NO: 1; (bbbbb) W, C at a residue corresponding to position 197 in SEQ ID NO: 1; (ccccc) Y, V, S, W, I, M, T at a residue corresponding to position 198 in SEQ ID NO: 1; (ddddd) M, L, W, P, F, C, A at a residue corresponding to position 199 in SEQ ID NO: 1; (eeeee) W at a residue corresponding to position 200 in SEQ ID NO: 1; (fffff) F, T, N, P, Y, L at a residue corresponding to position 201 in SEQ ID NO: 1; (ggggg) T, H, A, Q, E, L, G at a residue corresponding to position 202 in SEQ ID NO: 1; (hhhhh) T at a residue corresponding to position 205 in SEQ ID NO: 1; (iiiii) F at a residue corresponding to position 208 in SEQ ID NO: 1; (jjjjj) P at a residue corresponding to position 209 in SEQ ID NO: 1; (kkkkk) G at a residue corresponding to position 211 in SEQ ID NO: 1; (lllll) A, C at a residue corresponding to position 212 in SEQ ID NO: 1; (mmmmm) M, V, E at a residue corresponding to position 213 in SEQ ID NO: 1; (nnnnn) V at a residue corresponding to position 214 in SEQ ID NO: 1; (ooooo) S at a residue corresponding to position 215 in SEQ ID NO: 1; (ppppp) Q, R, Y, H, K, G at a residue corresponding to position 217 in SEQ ID NO: 1; (qqqqq) W, M, V at a residue corresponding to position 220 in SEQ ID NO: 1; (rrrrr) T, A at a residue corresponding to position 221 in SEQ ID NO: 1; (sssss) S, N at a residue corresponding to position 222 in SEQ ID NO: 1; (ttttt) A, N, I, L, H at a residue corresponding to position 223 in SEQ ID NO: 1; (uuuuu) T, Q, H, I, N, M at a residue corresponding to position 224 in SEQ ID NO: 1; (vvvvv) L, A, F, Q at a residue corresponding to position 225 in SEQ ID NO: 1; (wwwww) Y, C, T, W at a residue corresponding to position 226 in SEQ ID NO: 1; (xxxxx) G, E, M, T, A, GKWSQD (SEQ ID NO: 28), GKWSQSY (SEQ ID NO: 29), GKWSQ (SEQ ID NO: 30), GKWSQDLK (SEQ ID NO: 31), GKWSQQLQ (SEQ ID NO: 32) at a residue corresponding to position 227 in SEQ ID NO: 1; (yyyyy) a deletion at a residue corresponding to position 228 in SEQ ID NO: 1; (zzzzz) a deletion, F, S, L, G, W, I, M, H at a residue corresponding to position 229 in SEQ ID NO: 1; (aaaaaa) a deletion, R, M, P, T, E, Q, A at a residue corresponding to position 230 in SEQ ID NO: 1; (bbbbbb) a deletion, Q, P, H, QDLQ (SEQ ID NO: 33) at a residue corresponding to position 231 in SEQ ID NO: 1; (cccccc) a deletion, W, A, Y, N, K, Q, D at a residue corresponding to position 232 in SEQ ID NO: 1; (dddddd) a deletion, W, R, M, Y, P at a residue corresponding to position 233 in SEQ ID NO: 1; (eeeeee) a deletion, F, Y, T, K, R, Q, P, G, H, E at a residue corresponding to position 234 in SEQ ID NO: 1; (ffffff) H, S, R, K, M, Q, D, G at a residue corresponding to position 235 in SEQ ID NO: 1; (gggggg) P, N, H, T, D, DISAC (SEQ ID NO: 34), DVS at a residue corresponding to position 238 in SEQ ID NO: 1; (hhhhhh) V, F, R, a deletion at a residue corresponding to position 239 in SEQ ID NO: 1; (iiiiii) a deletion, F, H, T, W, S, L, C, V at a residue corresponding to position 240 in SEQ ID NO: 1; (jjjjjj) a deletion, C, M, P, I at a residue corresponding to position 241 in SEQ ID NO: 1; (kkkkkk) a deletion, G, V, C at a residue corresponding to position 242 in SEQ ID NO: 1; (llllll) L, C, M at a residue corresponding to position 243 in SEQ ID NO: 1; (mmmmmm) M, V at a residue corresponding to position 244 in SEQ ID NO: 1; (nnnnnn) W, F, L, Y at a residue corresponding to position 246 in SEQ ID NO: 1; (oooooo) F, C at a residue corresponding to position 247 in SEQ ID NO: 1; (pppppp) S, P at a residue corresponding to position 248 in SEQ ID NO: 1; (qqqqqq) A, V, M at a residue corresponding to position 249 in SEQ ID NO: 1; (rrrrrr) M, A, W at a residue corresponding to position 250 in SEQ ID NO: 1; (ssssss) A, H at a residue corresponding to position 251 in SEQ ID NO: 1; (tttttt) I, M, W, Q at a residue corresponding to position 252 in SEQ ID NO: 1; (uuuuuu) T, Q, N, I, H, V, M at a residue corresponding to position 253 in SEQ ID NO: 1; (vvvvvv) E, I, T, Y, H, V, L, P, A, M, F, S, G at a residue corresponding to position 254 in SEQ ID NO: 1; (wwwwww) M, T, I, N, Y, C, ILLANV (SEQ ID NO: 35), ILLANVHY (SEQ ID NO: 36), ILLLNVHY (SEQ ID NO: 37), ILLLNV (SEQ ID NO: 38), ILLLNVHK (SEQ ID NO: 39), ILLHNVHK (SEQ ID NO: 40), ILLSNVHY (SEQ ID NO: 41) at a residue corresponding to position 255 in SEQ ID NO: 1; (xxxxxx) I, V, H, C, F, a deletion at a residue corresponding to position 256 in SEQ ID NO: 1; (yyyyyy) a deletion, L, T, N at a residue corresponding to position 257 in SEQ ID NO: 1; (zzzzzz) a deletion, S, L, P, M, H, T, A at a residue corresponding to position 258 in SEQ ID NO: 1; (aaaaaaa) a deletion, C, A at a residue corresponding to position 259 in SEQ ID NO: 1; (bbbbbbb) a deletion, F, L, M, V at a residue corresponding to position 260 in SEQ ID NO: 1; (ccccccc) a deletion, Y, M, W, A, F, L, S at a residue corresponding to position 261 in SEQ ID NO: 1; (ddddddd) a deletion, M, K, Y, R, G, C, L, V, S, N at a residue corresponding to position 262 in SEQ ID NO: 1; (eeeeeee) G, W, I, S, V at a residue corresponding to position 264 in SEQ ID NO: 1; (fffffff) R, C, H, K at a residue corresponding to position 265 in SEQ ID NO: 1; (ggggggg) I, V, Q, C at a residue corresponding to position 266 in SEQ ID NO: 1; (hhhhhhh) Y at a residue corresponding to position 267 in SEQ ID NO: 1; (iiiiiii) V, M, T, F, IMPR (SEQ ID NO: 42), IMNR (SEQ ID NO: 43) at a residue corresponding to position 268 in SEQ ID NO: 1; (jjjjjjj) M, at a residue corresponding to position 269 in SEQ ID NO: 1; (kkkkkkk) a deletion, P at a residue corresponding to position 270 in SEQ ID NO: 1; (lllllll) a deletion, R, Q at a residue corresponding to position 271 in SEQ ID NO: 1; (mmmmmmm) W at a residue corresponding to position 272 in SEQ ID NO: 1; (nnnnnnn) P, PEEMLRAIQ (SEQ ID NO: 44), PEEMLQAIQ (SEQ ID NO: 45) at a residue corresponding to position 274 in SEQ ID NO: 1; (ooooooo) D, A, E, R, K, EEMLQAIQ (SEQ ID NO: 46), EEMLRAIQ (SEQ ID NO: 47), a deletion, KEMLQAIQ (SEQ ID NO: 48) at a residue corresponding to position 275 in SEQ ID NO: 1; (ppppppp) a deletion at a residue corresponding to position 276 in SEQ ID NO: 1; (qqqqqqq) a deletion, M, Y, L, F at a residue corresponding to position 277 in SEQ ID NO: 1; (rrrrrrr) a deletion at a residue corresponding to position 278 in SEQ ID NO: 1; (sssssss) a deletion, Q, R, QAIQ (SEQ ID NO: 49), RAIQ (SEQ ID NO: 50) at a residue corresponding to position 279 in SEQ ID NO: 1; (ttttttt) a deletion at a residue corresponding to position 280 in SEQ ID NO: 1; (uuuuuuu) a deletion, V, M, L, I at a residue corresponding to position 281 in SEQ ID NO: 1; (vvvvvvv) a deletion, Q at a residue corresponding to position 282 in SEQ ID NO: 1; (wwwwwww) P, C at a residue corresponding to position 286 in SEQ ID NO: 1; (xxxxxxx) T, TVFFA (SEQ ID NO: 51), TSFFA (SEQ ID NO: 52), TFFFA (SEQ ID NO: 53), TMFFA (SEQ ID NO: 54), TVFHA (SEQ ID NO: 55) at a residue corresponding to position 287 in SEQ ID NO: 1; (yyyyyyy) H, F, M, Y, R, I, V, T, C, a deletion at a residue corresponding to position 288 in SEQ ID NO: 1; (zzzzzzz) a deletion, M, V, A, L, F, I at a residue corresponding to position 289 in SEQ ID NO: 1; (aaaaaaaa) a deletion, A, W, H, C, F, V, P, L at a residue corresponding to position 290 in SEQ ID NO: 1; (bbbbbbbb) a deletion, A, M, G, V at a residue corresponding to position 291 in SEQ ID NO: 1; (cccccccc) A, T at a residue corresponding to position 294 in SEQ ID NO: 1; (dddddddd) L, a deletion at a residue corresponding to position 295 in SEQ ID NO: 1; (eeeeeeee) M, L at a residue corresponding to position 296 in SEQ ID NO: 1; (ffffffff) M, Q, I, A, L, T at a residue corresponding to position 297 in SEQ ID NO: 1; (gggggggg) M, A, F, R, IM at a residue corresponding to position 298 in SEQ ID NO: 1; (hhhhhhhh) M, F at a residue corresponding to position 299 in SEQ ID NO: 1; (iiiiiiii) A at a residue corresponding to position 300 in SEQ ID NO: 1; (jjjjjjjj) M, R, E at a residue corresponding to position 304 in SEQ ID NO: 1; (kkkkkkkk) D at a residue corresponding to position 306 in SEQ ID NO: 1; (llllllll) L at a residue corresponding to position 309 in SEQ ID NO: 1; (mmmmmmmm) L, D at a residue corresponding to position 310 in SEQ ID NO: 1; (nnnnnnnn) L at a residue corresponding to position 311 in SEQ ID NO: 1; (oooooooo) S, SHVRSV (SEQ ID NO: 56), SHVRTV (SEQ ID NO: 57) at a residue corresponding to position 314 in SEQ ID NO: 1; (pppppppp) S, a deletion at a residue corresponding to position 315 in SEQ ID NO: 1; (qqqqqqqq) a deletion, V, L, I at a residue corresponding to position 316 in SEQ ID NO: 1; (rrrrrrrr) a deletion, H, N, Q, T at a residue corresponding to position 317 in SEQ ID NO: 1; (ssssssss) a deletion, Y, M, T, S, C, Q, R, H, V, W, I, L at a residue corresponding to position 318 in SEQ ID NO: 1; (tttttttt) a deletion, C, F, V, L, M at a residue corresponding to position 319 in SEQ ID NO: 1; (uuuuuuuu) F, I, V, Y, T, M, W at a residue corresponding to position 320 in SEQ ID NO: 1; (vvvvvvvv) C at a residue corresponding to position 321 in SEQ ID NO: 1; (wwwwwwww) G at a residue corresponding to position 323 in SEQ ID NO: 1; (xxxxxxxx) S, M at a residue corresponding to position 324 in SEQ ID NO: 1; (yyyyyyyy) V, I, C at a residue corresponding to position 326 in SEQ ID NO: 1; (zzzzzzzz) S at a residue corresponding to position 327 in SEQ ID NO: 1; (aaaaaaaaa) H, V, R, S, RELE (SEQ ID NO: 58) at a residue corresponding to position 328 in SEQ ID NO: 1; (bbbbbbbbb) E, a deletion, ELE at a residue corresponding to position 329 in SEQ ID NO: 1; (ccccccccc) a deletion, L, T, I at a residue corresponding to position 330 in SEQ ID NO: 1; (ddddddddd) a deletion, M, E, C, L, I at a residue corresponding to position 331 in SEQ ID NO: 1; (eeeeeeeee) A, W, C, M, I at a residue corresponding to position 334 in SEQ ID NO: 1; (fffffffff) Q, R at a residue corresponding to position 335 in SEQ ID NO: 1; (ggggggggg) K at a residue corresponding to position 336 in SEQ ID NO: 1; (hhhhhhhhh) M at a residue corresponding to position 338 in SEQ ID NO: 1; (iiiiiiiii) S at a residue corresponding to position 339 in SEQ ID NO: 1; (jjjjjjjjj) H, T at a residue corresponding to position 341 in SEQ ID NO: 1; (kkkkkkkkk) L, F at a residue corresponding to position 342 in SEQ ID NO: 1; (lllllllll) D, H, Y at a residue corresponding to position 348 in SEQ ID NO: 1; (mmmmmmmmm) V, C, T, F, A, W at a residue corresponding to position 351 in SEQ ID NO: 1; (nnnnnnnnn) E, K, D at a residue corresponding to position 352 in SEQ ID NO: 1; (ooooooooo) V, L, Mat a residue corresponding to position 353 in SEQ IDNO: 1; (ppppppppp) C, F, L, Y, A, K, S, I at a residue corresponding to position 354 in SEQ ID NO: 1; (qqqqqqqqq) E at a residue corresponding to position 355 in SEQ ID NO: 1; (rrrrrrrrr) A at a residue corresponding to position 356 in SEQ ID NO: 1; (sssssssss) W at a residue corresponding to position 357 in SEQ ID NO: 1; (ttttttttt) M at a residue corresponding to position 359 in SEQ ID NO: 1; (uuuuuuuuu) S at a residue corresponding to position 360 in SEQ ID NO: 1; (vvvvvvvvv) C, S at a residue corresponding to position 362 in SEQ ID NO: 1; (wwwwwwwww) A, T, G at a residue corresponding to position 363 in SEQ ID NO: 1; (xxxxxxxxx) C, S at a residue corresponding to position 364 in SEQ ID NO: 1; (yyyyyyyyy) L, I, M, C at a residue corresponding to position 365 in SEQ ID NO: 1; (zzzzzzzzz) S, V, T at a residue corresponding to position 366 in SEQ ID NO: 1; (aaaaaaaaaa) A, C, S, H at a residue corresponding to position 367 in SEQ ID NO: 1; (bbbbbbbbbb) H at a residue corresponding to position 368 in SEQ ID NO: 1; (cccccccccc) H, Y, I, C, L, V, W, S, T, M at a residue corresponding to position 369 in SEQ ID NO: 1; (dddddddddd) Y, R, T, S, H, G, N, Q at a residue corresponding to position 370 in SEQ ID NO: 1; (eeeeeeeeee) W at a residue corresponding to position 371 in SEQ ID NO: 1; (ffffffffff) K, W, Q, H, S, R, N at a residue corresponding to position 372 in SEQ ID NO: 1; (gggggggggg) S, H, P, Q, E at a residue corresponding to position 373 in SEQ ID NO: 1; (hhhhhhhhhh) D, T, A, R, S, P, W, E, Q, H at a residue corresponding to position 374 in SEQ ID NO: 1; (iiiiiiiiii) P at a residue corresponding to position 375 in SEQ ID NO: 1; (jjjjjjjjjj) T, K, E, Q, H, D at a residue corresponding to position 376 in SEQ ID NO: 1; (kkkkkkkkkk) Y, R, N at a residue corresponding to position 377 in SEQ ID NO: 1; (llllllllll) S, Y, P, V, A at a residue corresponding to position 378 in SEQ ID NO: 1; (mmmmmmmmmm) G at a residue corresponding to position 379 in SEQ ID NO: 1; (nnnnnnnnnn) T at a residue corresponding to position 380 in SEQ ID NO: 1; (oooooooooo) V, S, I at a residue corresponding to position 381 in SEQ ID NO: 1; (pppppppppp) Q, H, T, Y, W, P at a residue corresponding to position 383 in SEQ ID NO: 1; (qqqqqqqqqq) I, P, A, M at a residue corresponding to position 384 in SEQ ID NO: 1; (rrrrrrrrrr) S, A at a residue corresponding to position 386 in SEQ ID NO: 1; (ssssssssss) G, S, H at a residue corresponding to position 387 in SEQ ID NO: 1; (tttttttttt) T, M, V, L at a residue corresponding to position 388 in SEQ ID NO: 1; (uuuuuuuuuu) R, K, E at a residue corresponding to position 389 in SEQ ID NO: 1; (vvvvvvvvvv) C, L, A at a residue corresponding to position 390 in SEQ ID NO: 1; (wwwwwwwwww) K at a residue corresponding to position 391 in SEQ ID NO: 1; (xxxxxxxxxx) D at a residue corresponding to position 394 in SEQ ID NO: 1; (yyyyyyyyyy) D, N at a residue corresponding to position 398 in SEQ ID NO: 1; (zzzzzzzzzz) V, L, I at a residue corresponding to position 402 in SEQ ID NO: 1; (aaaaaaaaaaa) L, V at a residue corresponding to position 406 in SEQ ID NO: 1; (bbbbbbbbbbb) D at a residue corresponding to position 407 in SEQ ID NO: 1; (ccccccccccc) N, Q, NTENAPR (SEQ ID NO: 59), NTENAPD (SEQ ID NO: 60) at a residue corresponding to position 410 in SEQ ID NO: 1; (ddddddddddd) T, TENAPD (SEQ ID NO: 61), TQNAPR (SEQ ID NO: 62), a deletion, TENAPR (SEQ ID NO: 63), TQNAPN (SEQ ID NO: 64) at a residue corresponding to position 411 in SEQ ID NO: 1; (eeeeeeeeeee) a deletion, Q at a residue corresponding to position 412 in SEQ ID NO: 1; (fffffffffff) a deletion, N, NNPN (SEQ ID NO: 65) at a residue corresponding to position 413 in SEQ ID NO: 1; (ggggggggggg) a deletion, A, S, N at a residue corresponding to position 414 in SEQ ID NO: 1; (hhhhhhhhhhh) a deletion, P at a residue corresponding to position 415 in SEQ ID NO: 1; (iiiiiiiiiii) a deletion, D, R, N, E at a residue corresponding to position 416 in SEQ ID NO: 1; (jjjjjjjjjjj) P at a residue corresponding to position 418 in SEQ ID NO: 1; (kkkkkkkkkkk) G at a residue corresponding to position 419 in SEQ ID NO: 1; (lllllllllll) E at a residue corresponding to position 420 in SEQ ID NO: 1; (mmmmmmmmmmm) M, V at a residue corresponding to position 424 in SEQ ID NO: 1; (nnnnnnnnnnn) V, L at a residue corresponding to position 425 in SEQ ID NO: 1; (ooooooooooo) M, L at a residue corresponding to position 426 in SEQ ID NO: 1; (ppppppppppp) T, N, S at a residue corresponding to position 430 in SEQ ID NO: 1; (qqqqqqqqqqq) I, L at a residue corresponding to position 431 in SEQ ID NO: 1; (rrrrrrrrrrr) T, F at a residue corresponding to position 432 in SEQ ID NO: 1; (sssssssssss) S, K at a residue corresponding to position 433 in SEQ ID NO: 1; (ttttttttttt) W at a residue corresponding to position 436 in SEQ ID NO: 1; (uuuuuuuuuuu) T at a residue corresponding to position 442 in SEQ ID NO: 1; (vvvvvvvvvvv) R, K, RASFA (SEQ ID NO: 66) at a residue corresponding to position 443 in SEQ ID NO: 1; (wwwwwwwwwww) a deletion at a residue corresponding to position 444 in SEQ ID NO: 1; (xxxxxxxxxxx) a deletion, M, T, S, A at a residue corresponding to position 445 in SEQ ID NO: 1; (yyyyyyyyyyy) a deletion at a residue corresponding to position 446 in SEQ ID NO: 1; (zzzzzzzzzzz) a deletion, A at a residue corresponding to position 447 in SEQ ID NO: 1; (aaaaaaaaaaaa) S, G at a residue corresponding to position 455 in SEQ ID NO: 1; (bbbbbbbbbbbb) G, E, D at a residue corresponding to position 459 in SEQ ID NO: 1; (cccccccccccc) D, G at a residue corresponding to position 461 in SEQ ID NO: 1; (dddddddddddd) R, G at a residue corresponding to position 462 in SEQ ID NO: 1; (eeeeeeeeeeee) L, Y at a residue corresponding to position 464 in SEQ ID NO: 1; (ffffffffffff) H at a residue corresponding to position 465 in SEQ ID NO: 1; (gggggggggggg) M at a residue corresponding to position 469 in SEQ ID NO: 1; (hhhhhhhhhhhh) C, G at a residue corresponding to position 470 in SEQ ID NO: 1; (iiiiiiiiiiii) Y, K, M, W, I, R at a residue corresponding to position 471 in SEQ ID NO: 1; (jjjjjjjjjjjj) I, F, C, Y, L, M at a residue corresponding to position 472 in SEQ ID NO: 1; (kkkkkkkkkkkk) E, EDGH (SEQ ID NO: 67), EDGY (SEQ ID NO: 68) at a residue corresponding to position 474 in SEQ ID NO: 1; (llllllllllll) D, a deletion at a residue corresponding to position 475 in SEQ ID NO: 1; (mmmmmmmmmmmm) a deletion at a residue corresponding to position 476 in SEQ ID NO: 1; (nnnnnnnnnnnn) a deletion, H, F, N, E, Y at a residue corresponding to position 477 in SEQ ID NO: 1; (oooooooooooo) W, I, M at a residue corresponding to position 478 in SEQ ID NO: 1; (pppppppppppp) T, H, Y, F, W at a residue corresponding to position 479 in SEQ ID NO: 1; (qqqqqqqqqqqq) V, L at a residue corresponding to position 480 in SEQ ID NO: 1; (rrrrrrrrrrrr) C, I, T at a residue corresponding to position 481 in SEQ ID NO: 1; (ssssssssssss) L at a residue corresponding to position 484 in SEQ ID NO: 1; (tttttttttttt) D at a residue corresponding to position 486 in SEQ ID NO: 1; (uuuuuuuuuuuu) I at a residue corresponding to position 489 in SEQ ID NO: 1; (vvvvvvvvvvvv) R, C, V at a residue corresponding to position 490 in SEQ ID NO: 1; (wwwwwwwwwwww) G, R, N at a residue corresponding to position 491 in SEQ ID NO: 1; (xxxxxxxxxxxx) F, H, Y, L at a residue corresponding to position 493 in SEQ ID NO: 1; (yyyyyyyyyyyy) N at a residue corresponding to position 494 in SEQ ID NO: 1; (zzzzzzzzzzzz) F, S, Y at a residue corresponding to position 496 in SEQ ID NO: 1; (aaaaaaaaaaaaa) L, V, I at a residue corresponding to position 503 in SEQ ID NO: 1; (bbbbbbbbbbbbb) R, RHPD (SEQ ID NO: 69) at a residue corresponding to position 506 in SEQ ID NO: 1; (ccccccccccccc) a deletion at a residue corresponding to position 507 in SEQ ID NO: 1; (ddddddddddddd) a deletion, P at a residue corresponding to position 508 in SEQ ID NO: 1; (eeeeeeeeeeeee) a deletion, D at a residue corresponding to position 509 in SEQ ID NO: 1; (fffffffffffff) A, V, C at a residue corresponding to position 513 in SEQ ID NO: 1; (ggggggggggggg) V at a residue corresponding to position 518 in SEQ ID NO: 1; (hhhhhhhhhhhhh) P at a residue corresponding to position 519 in SEQ ID NO: 1; (iiiiiiiiiiiii) E, P at a residue corresponding to position 521 in SEQ ID NO: 1; (jjjjjjjjjjjjj) Q, A, R, M, Y, D at a residue corresponding to position 523 in SEQ ID NO: 1; (kkkkkkkkkkkkk) S, A at a residue corresponding to position 524 in SEQ ID NO: 1; (lllllllllllll) S, T, D, G, E at a residue corresponding to position 525 in SEQ ID NO: 1; (mmmmmmmmmmmmm) P, T, V at a residue corresponding to position 527 in SEQ ID NO: 1; (nnnnnnnnnnnnn) A, V at a residue corresponding to position 528 in SEQ ID NO: 1; (ooooooooooooo) V, A at a residue corresponding to position 529 in SEQ ID NO: 1; (ppppppppppppp) L, V at a residue corresponding to position 539 in SEQ ID NO: 1; (qqqqqqqqqqqqq) T at a residue corresponding to position 540 in SEQ ID NO: 1; (rrrrrrrrrrrrr) E, D at a residue corresponding to position 542 in SEQ ID NO: 1; (sssssssssssss) A, K at a residue corresponding to position 546 in SEQ ID NO: 1; (ttttttttttttt) K, R at a residue corresponding to position 551 in SEQ ID NO: 1; (uuuuuuuuuuuuu) S at a residue corresponding to position 553 in SEQ ID NO: 1; (vvvvvvvvvvvvv) S, P, H at a residue corresponding to position 554 in SEQ ID NO: 1; (wwwwwwwwwwwww) F at a residue corresponding to position 555 in SEQ ID NO: 1; (xxxxxxxxxxxxx) K at a residue corresponding to position 557 in SEQ ID NO: 1; (yyyyyyyyyyyyy) P at a residue corresponding to position 559 in SEQ ID NO: 1; (zzzzzzzzzzzzz) G at a residue corresponding to position 560 in SEQ ID NO: 1; (aaaaaaaaaaaaaa) V, a deletion at a residue corresponding to position 562 in SEQ ID NO: 1; (bbbbbbbbbbbbbb) F at a residue corresponding to position 564 in SEQ ID NO: 1; (cccccccccccccc) V, F, FV at a residue corresponding to position 565 in SEQ ID NO: 1; (dddddddddddddd) E, VE at a residue corresponding to position 566 in SEQ ID NO: 1; (eeeeeeeeeeeeee) Q, E at a residue corresponding to position 583 in SEQ ID NO: 1; (ffffffffffffff) or any combination thereof.

In some embodiments, an engineered ACL described herein having one or more amino acid alterations results in an engineered ACL comprising: (a) K, S at a residue corresponding to position 7 in SEQ ID NO: 1; (b) S at a residue corresponding to position 8 in SEQ ID NO: 1; (c) W, Y, F at a residue corresponding to position 10 in SEQ ID NO: 1; (d) E, P, D at a residue corresponding to position 12 in SEQ ID NO: 1; (e) I at a residue corresponding to position 15 in SEQ ID NO: 1; (f) E at a residue corresponding to position 16 in SEQ ID NO: 1; (g) F at a residue corresponding to position 19 in SEQ ID NO: 1; (h) R, K at a residue corresponding to position 52 in SEQ ID NO: 1; (i) L at a residue corresponding to position 54 in SEQ ID NO: 1; (j) A at a residue corresponding to position 64 in SEQ ID NO: 1; (k) R at a residue corresponding to position 66 in SEQ ID NO: 1; (1) A at a residue corresponding to position 67 in SEQ ID NO: 1; (m) L at a residue corresponding to position 73 in SEQ ID NO: 1; (n) K at a residue corresponding to position 74 in SEQ ID NO: 1; (o) T at a residue corresponding to position 82 in SEQ ID NO: 1; (p) I at a residue corresponding to position 87 in SEQ ID NO: 1; (q) L, M at a residue corresponding to position 90 in SEQ ID NO: 1; (r) T at a residue corresponding to position 94 in SEQ ID NO: 1; (s) L at a residue corresponding to position 100 in SEQ ID NO: 1; (t) V at a residue corresponding to position 111 in SEQ ID NO: 1; (u) N at a residue corresponding to position 115 in SEQ ID NO: 1; (v) S at a residue corresponding to position 117 in SEQ ID NO: 1; (w) T at a residue corresponding to position 119 in SEQ ID NO: 1; (x) D at a residue corresponding to position 121 in SEQ ID NO: 1; (y) I at a residue corresponding to position 129 in SEQ ID NO: 1; (z) G at a residue corresponding to position 131 in SEQ ID NO: 1; (aa) D, S at a residue corresponding to position 138 in SEQ ID NO: 1; (bb) E at a residue corresponding to position 139 in SEQ ID NO: 1; (cc) K, E, D at a residue corresponding to position 140 in SEQ ID NO: 1; (dd) T at a residue corresponding to position 144 in SEQ ID NO: 1; (ee) L, I at a residue corresponding to position 150 in SEQ ID NO: 1; (ff) S at a residue corresponding to position 165 in SEQ ID NO: 1; (gg) F at a residue corresponding to position 166 in SEQ ID NO: 1; (hh) L, A, D at a residue corresponding to position 172 in SEQ ID NO: 1; (ii) Q at a residue corresponding to position 182 in SEQ ID NO: 1; (jj) A at a residue corresponding to position 191 in SEQ ID NO: 1; (kk) A at a residue corresponding to position 199 in SEQ ID NO: 1; (ll) P at a residue corresponding to position 209 in SEQ ID NO: 1; (mm) C at a residue corresponding to position 212 in SEQ ID NO: 1; (nn) R at a residue corresponding to position 217 in SEQ ID NO: 1; (oo) T, A at a residue corresponding to position 221 in SEQ ID NO: 1; (pp) N, I, A, L, H at a residue corresponding to position 223 in SEQ ID NO: 1; (qq) Q at a residue corresponding to position 224 in SEQ ID NO: 1; (rr) E, T, A at a residue corresponding to position 227 in SEQ ID NO: 1; (ss) I, L, M at a residue corresponding to position 229 in SEQ ID NO: 1; (tt) Q, A at a residue corresponding to position 230 in SEQ ID NO: 1; (uu) H, Q, P at a residue corresponding to position 231 in SEQ ID NO: 1; (vv) Y, K, Q, N at a residue corresponding to position 232 in SEQ ID NO: 1; (ww) W, Y at a residue corresponding to position 233 in SEQ ID NO: 1; (xx) Y, F, H at a residue corresponding to position 234 in SEQ ID NO: 1; (yy) S at a residue corresponding to position 235 in SEQ ID NO: 1; (zz) D, H, T at a residue corresponding to position 238 in SEQ ID NO: 1; (aaa) T, H, C, S at a residue corresponding to position 240 in SEQ ID NO: 1; (bbb) C, M at a residue corresponding to position 243 in SEQ ID NO: 1; (ccc) M, V at a residue corresponding to position 249 in SEQ ID NO: 1; (ddd) H, M, Q at a residue corresponding to position 253 in SEQ ID NO: 1; (eee) M at a residue corresponding to position 254 in SEQ ID NO: 1; (fff) I, V at a residue corresponding to position 256 in SEQ ID NO: 1; (ggg) T, H, A, L, M at a residue corresponding to position 258 in SEQ ID NO: 1; (hhh) A at a residue corresponding to position 259 in SEQ ID NO: 1; (iii) K, C, N at a residue corresponding to position 262 in SEQ ID NO: 1; (jjj) F at a residue corresponding to position 268 in SEQ ID NO: 1; (kkk) R, Q at a residue corresponding to position 271 in SEQ ID NO: 1; (lll) E at a residue corresponding to position 275 in SEQ ID NO: 1; (mmm) L, M at a residue corresponding to position 277 in SEQ ID NO: 1; (nnn) V at a residue corresponding to position 281 in SEQ ID NO: 1; (ooo) V, I, F, T, H, Y at a residue corresponding to position 288 in SEQ ID NO: 1; (ppp) V, A at a residue corresponding to position 290 in SEQ ID NO: 1; (qqq) L, M at a residue corresponding to position 296 in SEQ ID NO: 1; (rrr) I at a residue corresponding to position 297 in SEQ ID NO: 1; (sss) A at a residue corresponding to position 300 in SEQ ID NO: 1; (ttt) E at a residue corresponding to position 304 in SEQ ID NO: 1; (uuu) D at a residue corresponding to position 306 in SEQ ID NO: 1; (vvv) L at a residue corresponding to position 316 in SEQ ID NO: 1; (www) S, R, H, W, Y, M, T, C, Q, V, I, L at a residue corresponding to position 318 in SEQ ID NO: 1; (xxx) M, F, T, Y, W at a residue corresponding to position 320 in SEQ ID NO: 1; (yyy) C at a residue corresponding to position 321 in SEQ ID NO: 1; (zzz) G at a residue corresponding to position 323 in SEQ ID NO: 1; (aaaa) V, H, R at a residue corresponding to position 328 in SEQ ID NO: 1; (bbbb) I, L at a residue corresponding to position 330 in SEQ ID NO: 1; (cccc) I at a residue corresponding to position 331 in SEQ ID NO: 1; (dddd) K at a residue corresponding to position 336 in SEQ ID NO: 1; (eeee) K at a residue corresponding to position 352 in SEQ ID NO: 1; (ffff) V, L at a residue corresponding to position 353 in SEQ ID NO: 1; (gggg) G at a residue corresponding to position 363 in SEQ ID NO: 1; (hhhh) S at a residue corresponding to position 364 in SEQ ID NO: 1; (iiii) C, L, I, M at a residue corresponding to position 365 in SEQ ID NO: 1; (jjjj) V at a residue corresponding to position 366 in SEQ ID NO: 1; (kkkk) H, S, T, M at a residue corresponding to position 369 in SEQ ID NO: 1; (llll) S, K, H, N at a residue corresponding to position 372 in SEQ ID NO: 1; (mmmm) Q, R, T, P, H, S, A, D, E at a residue corresponding to position 374 in SEQ ID NO: 1; (nnnn) T at a residue corresponding to position 376 in SEQ ID NO: 1; (oooo) A, P, S, V at a residue corresponding to position 378 in SEQ ID NO: 1; (pppp) T at a residue corresponding to position 388 in SEQ ID NO: 1; (qqqq) C, A at a residue corresponding to position 390 in SEQ ID NO: 1; (rrrr) D at a residue corresponding to position 394 in SEQ ID NO: 1; (ssss) N at a residue corresponding to position 398 in SEQ ID NO: 1; (tttt) L at a residue corresponding to position 406 in SEQ ID NO: 1; (uuuu) D at a residue corresponding to position 407 in SEQ ID NO: 1; (vvvv) N, E at a residue corresponding to position 416 in SEQ ID NO: 1; (wwww) F at a residue corresponding to position 432 in SEQ ID NO: 1; (xxxx) R, K at a residue corresponding to position 443 in SEQ ID NO: 1; (yyyy) A at a residue corresponding to position 445 in SEQ ID NO: 1; (zzzz) S, G at a residue corresponding to position 455 in SEQ ID NO: 1; (aaaaa) R at a residue corresponding to position 462 in SEQ ID NO: 1; (bbbbb) M at a residue corresponding to position 469 in SEQ ID NO: 1; (ccccc) G at a residue corresponding to position 470 in SEQ ID NO: 1; (ddddd) R, M, W at a residue corresponding to position 471 in SEQ ID NO: 1; (eeeee) V at a residue corresponding to position 480 in SEQ ID NO: 1; (fffff) I, C at a residue corresponding to position 481 in SEQ ID NO: 1; (ggggg) L at a residue corresponding to position 484 in SEQ ID NO: 1; (hhhhh) D at a residue corresponding to position 486 in SEQ ID NO: 1; (iiiii) C at a residue corresponding to position 490 in SEQ ID NO: 1; (jjjjj) R, G at a residue corresponding to position 491 in SEQ ID NO: 1; (kkkkk) L, H, Y at a residue corresponding to position 493 in SEQ ID NO: 1; (lllll) N at a residue corresponding to position 494 in SEQ ID NO: 1; (mmmmm) Y at a residue corresponding to position 496 in SEQ ID NO: 1; (nnnnn) P at a residue corresponding to position 508 in SEQ ID NO: 1; (ooooo) A, V, C at a residue corresponding to position 513 in SEQ ID NO: 1; (ppppp) V at a residue corresponding to position 518 in SEQ ID NO: 1; (qqqqq) P at a residue corresponding to position 519 in SEQ ID NO: 1; (rrrrr) E, P at a residue corresponding to position 521 in SEQ ID NO: 1; (sssss) R, Q, A, Y, M, D at a residue corresponding to position 523 in SEQ ID NO: 1; (ttttt) D, S, T, G, E at a residue corresponding to position 525 in SEQ ID NO: 1; (uuuuu) K at a residue corresponding to position 546 in SEQ ID NO: 1; (vvvvv) H, S, P at a residue corresponding to position 554 in SEQ ID NO: 1; (wwwww) F at a residue corresponding to position 555 in SEQ ID NO: 1; (xxxxx) G at a residue corresponding to position 560 in SEQ ID NO: 1; (yyyyy) V at a residue corresponding to position 565 in SEQ ID NO: 1; (zzzzz) Q, E at a residue corresponding to position 583 in SEQ ID NO: 1; (aaaaaa) or any combination thereof.

In some embodiments, an engineered ACL described herein having one or more amino acid alterations results in an engineered ACL comprising: (a) F at a residue corresponding to position 10 in SEQ ID NO: 1; (b) A at a residue corresponding to position 199 in SEQ ID NO: 1; (c) L, H, A at a residue corresponding to position 223 in SEQ ID NO: 1; (d) M at a residue corresponding to position 229 in SEQ ID NO: 1; (e) Q, A at a residue corresponding to position 230 in SEQ ID NO: 1; (f) P at a residue corresponding to position 231 in SEQ ID NO: 1; (g) N at a residue corresponding to position 232 in SEQ ID NO: 1; (h) W, Y at a residue corresponding to position 233 in SEQ ID NO: 1; (i) H at a residue corresponding to position 234 in SEQ ID NO: 1; (j) S at a residue corresponding to position 235 in SEQ ID NO: 1; (k) T at a residue corresponding to position 238 in SEQ ID NO: 1; (l) C, M at a residue corresponding to position 243 in SEQ ID NO: 1; (m) I, V at a residue corresponding to position 256 in SEQ ID NO: 1; (n) L, M at a residue corresponding to position 258 in SEQ ID NO: 1; (o) M at a residue corresponding to position 277 in SEQ ID NO: 1; (p) Y, H at a residue corresponding to position 288 in SEQ ID NO: 1; (q) A at a residue corresponding to position 300 in SEQ ID NO: 1; (r) V, T, C, I, L, Q, M, Y at a residue corresponding to position 318 in SEQ ID NO: 1; (s) W, Y at a residue corresponding to position 320 in SEQ ID NO: 1; (t) C at a residue corresponding to position 321 in SEQ ID NO: 1; (u) G at a residue corresponding to position 323 in SEQ ID NO: 1; (v) R at a residue corresponding to position 328 in SEQ ID NO: 1; (w) L at a residue corresponding to position 330 in SEQ ID NO: 1; (x) S at a residue corresponding to position 364 in SEQ ID NO: 1; (y) I, M at a residue corresponding to position 365 in SEQ ID NO: 1; (z) M at a residue corresponding to position 369 in SEQ ID NO: 1; (aa) N at a residue corresponding to position 372 in SEQ ID NO: 1; (bb) E at a residue corresponding to position 374 in SEQ ID NO: 1; (cc) V at a residue corresponding to position 378 in SEQ ID NO: 1; (dd) R at a residue corresponding to position 462 in SEQ ID NO: 1; (ee) M at a residue corresponding to position 469 in SEQ ID NO: 1; (ff) V at a residue corresponding to position 480 in SEQ ID NO: 1; (gg) C at a residue corresponding to position 481 in SEQ ID NO: 1; (hh) L at a residue corresponding to position 484 in SEQ ID NO: 1; (ii) C, A, V at a residue corresponding to position 513 in SEQ ID NO: 1; (jj) D at a residue corresponding to position 523 in SEQ ID NO: 1; (kk) G, E, T at a residue corresponding to position 525 in SEQ ID NO: 1; (ll) S, P at a residue corresponding to position 554 in SEQ ID NO: 1; (mm) G at a residue corresponding to position 560 in SEQ ID NO: 1; (nn) E at a residue corresponding to position 583 in SEQ ID NO: 1; or (oo) any combination thereof.

In some embodiments, an engineered ACL described herein having one or more amino acid alterations results in an engineered ACL comprising: (a) L, H at a residue corresponding to position 223 in SEQ ID NO: 1; (b) N at a residue corresponding to position 232 in SEQ ID NO: 1; (c) W, Y at a residue corresponding to position 233 in SEQ ID NO: 1; (d) H at a residue corresponding to position 234 in SEQ ID NO: 1; (e) T at a residue corresponding to position 238 in SEQ ID NO: 1; (f) L at a residue corresponding to position 258 in SEQ ID NO: 1; (g) Y, H at a residue corresponding to position 288 in SEQ ID NO: 1; (h) V, T, C, I, L at a residue corresponding to position 318 in SEQ ID NO: 1; (i) W, Y at a residue corresponding to position 320 in SEQ ID NO: 1; (j) C at a residue corresponding to position 321 in SEQ ID NO: 1; (k) G at a residue corresponding to position 323 in SEQ ID NO: 1; (l) S at a residue corresponding to position 364 in SEQ ID NO: 1; (m) C, A at a residue corresponding to position 513 in SEQ ID NO: 1; (n) D at a residue corresponding to position 523 in SEQ ID NO: 1; or (o) any combination thereof.

In some embodiments, an engineered ACL described herein includes one or more amino acid alterations selected from the group consisting of: I4M, N7D, N7K, N7Q, N7S, N7T, A8I, A8S, T9A, T9K, T9S, I10F, I10H, I10P, I10V, I10W, I10Y, N12D, N12E, N12H, N12P, I13C, I13M, V15D, V15I, V15L, V15P, D16E, D16H, D16N, Y19F, Y19L, Y19P, Y19Q, D20W, K22S, G30D, G31A, E35G, E35P, T36H, S39A, V40C, V40F, V40L, V40M, A43G, K44D, K44H, K44S, K44T, Q47R, Q47RTYTQKEL (“RTYTQKEL” disclosed as SEQ ID NO: 15), Q47RTYTQREL (“RTYTQREL” disclosed as SEQ ID NO: 16), Q47RTYT (“RTYT” disclosed as SEQ ID NO: 17), d48, d49, L49Y, d50, S50T, d51, Q51F, Q51H, Q51L, Q51R, Q51W, Q51Y, d52, A52K, A52R, d53, d54, M54L, W55L, K60R, Y61F, Y61L, L64A, L64G, S66R, S67A, Y69L, C72G, C72T, R73F, R73G, R73L, R73V, H74K, D76T, E78V, D80G, P81PG, S82A, S82E, S82G, S82N, S82R, S82T, d83, M83K, M83P, M83R, M83S, V86I, L87I, L87V, L87VAV, d88, I88A, d89, T89I, T89L, T89V, F90C, F90L, F90M, F90V, F90W, F91A, F91L, F91M, F91S, V94C, V94CIEAA (“CIEAA” disclosed as SEQ ID NO: 18), V94S, V94T, V94TIEAA (“TIEAA” disclosed as SEQ ID NO: 19), d95, d96, L96E, d97, d98, P98A, P98L, V99C, V99M, V100S, V100M, V100L, H101F, H101M, W102H, W102F, W102L, A103G, A104V, A104L, A104T, L105V, L105W, L105H, L105A, L105I, D106S, D106Y, D106R, D106K, L107A, L107C, L107V, A109L, A109V, A109Q, 111V, I111A, I111M, I111L, I111C, I111VTPAN (“VTPAN” disclosed as SEQ ID NO: 20), S112T, d112, d113, P113L, d114, G114A, G114L, G114V, d115, S115N, T116A, T116W, T116P, G117S, S119T, Q121R, Q121D, Q126H, F127M, F127L, F127MRHTG (“MRHTG” disclosed as SEQ ID NO: 21), F127MRITG (“MRITG” disclosed as SEQ ID NO: 22), d128, d129, V129I, V129H, d130, d131, T131G, T131D, V136L, Y137T, A138D, A138S, K139E, A140E, A140K, A140D, V144T, T149R, K150I, K150L, Y152A, Y152T, Y152C, Y152I, N153G, N153S, A159G, E162S, E162H, E162R, E162SIDAF (“SIDAF” disclosed as SEQ IDNO: 23), E162RIESF (“RIESF” disclosed as SEQ ID NO: 24), E162HFDSF (“HFDSF” disclosed as SEQ ID NO: 25), E162HFDAF (“HFDAF” disclosed as SEQ ID NO: 26), E162HIDSF (“HIDSF” disclosed as SEQ ID NO: 27), L163F, L163I, d163, d164, E164D, d165, A165S, d166, L166F, D167L, A170G, M172L, M172A, M172D, R182Q, H189E, H189G, S191A, R194D, I195T, I195P, I195M, A196T, A196I, Y197W, Y197C, L198Y, L198V, L198S, L198W, L198I, L198M, L198T, G199M, G199L, G199W, G199P, G199F, G199C, G199A, M200W, S201F, S201T, S201N, S201P, S201Y, S201L, S202T, S202H, S202A, S202Q, S202E, S202L, S202G, S205T, V208F, S209P, A211G, V212A, V212C, R213M, R213V, R213E, L214V, T215S, S217Q, S217R, S217Y, S217H, S217K, S217G, T220W, T220M, T220V, S221T, S221A, C222S, C222N, S223A, S223N, S223I, S223L, S223H, E224T, E224Q, E224H, E224I, E224N, E224M, I225L, I225A, I225F, I225Q, S226Y, S226C, S226T, S226W, N227G, N227E, N227M, N227T, N227A, N227GKWSQD (“GKWSQD” disclosed as SEQ ID NO: 28), N227GKWSQSY (“GKWSQSY” disclosed as SEQ ID NO: 29), N227GKWSQ (“GKWSQ” disclosed as SEQ ID NO: 30), N227GKWSQDLK (“GKWSQDLK” disclosed as SEQ ID NO: 31), N227GKWSQQLQ (“GKWSQQLQ” disclosed as SEQ ID NO: 32), d228, d229, A229F, A229S, A229L, A229G, A229W, A229I, A229M, A229H, d230, S230R, S230M, S230P, S230T, S230E, S230Q, S230A, d231, E231Q, E231P, E231H, E231QDLQ (“QDLQ” disclosed as SEQ ID NO: 33), d232, S232W, S232A, S232Y, S232N, S232K, S232Q, S232D, d233, L233W, L233R, L233M, L233Y, L233P, d234, A234F, A234Y, A234T, A234K, A234R, A234Q, A234P, A234G, A234H, A234E, T235H, T235S, T235R, T235K, T235M, T235Q, T235D, T235G, Q238P, Q238N, Q238H, Q238T, Q238D, Q238DISAC (“DISAC” disclosed as SEQ ID NO: 34), Q238DVS, I239V, I239F, I239R, d239, d240, A240F, A240H, A240T, A240W, A240S, A240L, A240C, A240V, d241, A241C, A241M, A241P, A241I, d242, A242G, A242V, A242C, I243L, I243C, I243M, I244M, I244V, V246W, V246F, V246L, V246Y, S247F, S247C, H248S, H248P, L249A, L249V, L249M, F250M, F250A, F250W, G251A, G251H, L252I, L252M, L252W, L252Q, S253T, S253Q, S253N, S253I, S253H, S253V, S253M, K254E, K254I, K254T, K254Y, K254H, K254V, K254L, K254P, K254A, K254M, K254F, K254S, K254G, F255M, F255T, F255I, F255N, F255Y, F255C, F255ILLANV (“ILLANV” disclosed as SEQ ID NO: 35), F255ILLANVHY (“ILLANVHY” disclosed as SEQ ID NO: 36), F255ILLLNVHY (“ILLLNVHY” disclosed as SEQ ID NO: 37), F255ILLLNV (“ILLLNV” disclosed as SEQ ID NO: 38), F255ILLLNVHK (“ILLLNVHK” disclosed as SEQ ID NO: 39), F255ILLHNVHK (“ILLHNVHK” disclosed as SEQ ID NO: 40), F255ILLSNVHY (“ILLSNVHY” disclosed as SEQ ID NO: 41), L256I, L256V, L256H, L256C, L256F, d256, d257, I257L, I257T, I257N, d258, G258S, G258L, G258P, G258M, G258H, G258T, G258A, d259, N259C, N259A, d260, P260F, P260L, P260M, P260V, d261, H261Y, H261M, H261W, H261A, H261F, H261L, H261S, d262, A262M, A262K, A262Y, A262R, A262G, A262C, A262L, A262V, A262S, A262N, A264G, A264W, A264I, A264S, A264V, T265R, T265C, T265H, T265K, T266I, T266V, T266Q, T266C, V267Y, Y268V, Y268M, Y268T, Y268F, Y268IMPR (“IMPR” disclosed as SEQ ID NO: 42), Y268EIMNR (“IMNR” disclosed as SEQ ID NO: 43), H269M, d269, d270, N270P, d271, G271R, G271Q, F272W, L274P, L274PEEMLRAIQ (“PEEMLRAIQ” disclosed as SEQ ID NO: 44), L274PEEMLQAIQ (“PEEMLQAIQ” disclosed as SEQ ID NO: 45), I275D, I275A, 1275E, I275R, I275K, I275EEMLQAIQ (“EEMLQAIQ” disclosed as SEQ ID NO: 46), I275EEMLRAIQ (“EEMLRAIQ” disclosed as SEQ ID NO: 47), d275-282, I275KEMLQAIQ (“KEMLQAIQ” disclosed as SEQ ID NO: 48), d276, d277, V277M, V277Y, V277L, V277F, d278, d279, E279Q, E279R, E279QAIQ (“QAIQ” disclosed as SEQ ID NO: 49), E279RAIQ (“RAIQ” disclosed as SEQ ID NO: 50), d280, d281, Q281V, Q281M, Q281L, Q281I, d282, K282Q, V286P, V286C, N287T, N287TVFFA (“TVFFA” disclosed as SEQ ID NO: 51), N287TSFFA (“TSFFA” disclosed as SEQ ID NO: 52), N287TFFFA (“TFFFA” disclosed as SEQ ID NO: 53), N287TMFFA (“TMFFA” disclosed as SEQ ID NO: 54), N287TVFHA (“TVFHA” disclosed as SEQ ID NO: 55), S288H, S288F, S288M, S288Y, S288R, S288I, S288V, S288T, S288C, d288, d289, W289M, W289V, W289A, W289L, W289F, W289I, d290, T290A, T290W, T290H, T290C, T290F, T290V, T290P, T290L, d291, L291A, L291M, L291G, L291V, P294A, P294T, I295L, d295, I296M, I296L, V297M, V297Q, V297I, V297A, V297L, V297T, L298M, L298A, L298F, L298R, L2981M, L299M, L299F, T300A, I304M, I304R, 1304E, E306D, I309L, P310L, P310D, S311L, A314S, A314SHVRSV (“SHVRSV” disclosed as SEQ ID NO: 56), A314SHVRTV (“SHVRTV” disclosed as SEQ ID NO: 57), H315S, d315, d316, M316V, M316L, M316I, d317, R317H, R317N, R317Q, R317T, d318, A318Y, A318M, A318T, A318S, A318C, A318Q, A318R, A318H, A318V, A318W, A318I, A318L, d319, I319C, I319F, I319V, I319L, I319M, L320F, L320I, L320V, L320Y, L320T, L320M, L320W, S321C, A323G, A324S, A324M, L326V, L326I, L326C, G327S, A328H, A328V, A328R, A328S, A328RELE (“RELE” disclosed as SEQ ID NO: 58), N329E, d329, N329ELE, d330, V330L, V330T, V330I, d331, T331M, T331E, T331C, T331L, T331I, L334A, L334W, L334C, L334M, L334I, L335Q, L335R, T336K, V338M, T339S, N341H, N341T, K342L, K342F, E348D, E348H, E348Y, L351V, L351C, L351T, L351F, L351A, L351W, R352E, R352K, R352D, I353V, I353L, I353M, V354C, V354F, V354L, V354Y, V354A, V354K, V354S, V354I, Q355E, G356A, Y357W, L359M, T360S, T362C, T362S, S363A, S363T, S363G, P364C, P364S, V365L, V365I, V365M, V365C, A366S, A366V, A366T, T367A, T367C, T367S, T367H, L368H, F369H, F369Y, F369I, F369C, F369L, F369V, F369W, F369S, F369T, F369M, D370Y, D370R, D370T, D370S, D370H, D370G, D370N, D370Q, P371W, E372K, E372W, E372Q, E372H, E372S, E372R, E372N, D373S, D373H, D373P, D373Q, D373E, K374D, K374T, K374A, K374R, K374S, K374P, K374W, K374E, K374Q, K374H, E375P, R376T, R376K, R376E, R376Q, R376H, R376D, H377Y, H377R, H377N, I378S, 1378Y, 1378P, 1378V, 1378A, R379G, S380T, C381V, C381S, C381I, K383Q, K383H, K383T, K383Y, K383W, K383P, L384I, L384P, L384A, L384M, P386S, P386A, N387G, N387S, N387H, S388T, S388M, S388V, S388L, Q389R, Q389K, Q389E, V390C, V390L, V390A, R391K, N394D, V398D, V398N, A402V, A402L, A402I, D406L, D406V, P407D, L410N, L410Q, L410NTENAPR (“NTENAPR” disclosed as SEQ ID NO: 59), L410NTENAPD (“NTENAPD” disclosed as SEQ ID NO: 60), D411T, D411TENAPD (“TENAPD” disclosed as SEQ ID NO: 61), D411TQNAPR (“TQNAPR” disclosed as SEQ ID NO: 62), d411, D411TENAPR (“TENAPR” disclosed as SEQ ID NO: 63), D411TQNAPN (“TQNAPN” disclosed as SEQ ID NO: 64), d412, E412Q, d413, A413N, A413NNPN (“NNPN” disclosed as SEQ ID NO: 65), d414, I414A, I414S, I414N, d415, K415P, d416, Q416D, Q416R, Q416N, Q416E, T418P, L419G, P420E, L424M, L424V, W425V, W425L, I426M, I426L, Q430T, Q430N, Q430S, V431I, V431L, M432T, M432F, D433S, D433K, H436W, N442T, E443R, E443K, E443RASFA (“RASFA” disclosed as SEQ ID NO: 66), d444, d445, C445M, C445T, C445S, C445A, d446, d447, V447A, D455S, D455G, Y459G, Y459E, Y459D, R461D, R461G, H462R, H462G, F464L, F464Y, R465H, V469M, A470C, A470G, L471Y, L471K, L471M, L471W, L471I, L471R, V472I, V472F, V472C, V472Y, V472L, V472M, K474E, K474EDGH (“EDGH” disclosed as SEQ ID NO: 67), K474EDGY (“EDGY” disclosed as SEQ ID NO: 68), Q475D, d475, d476, d477, R477H, R477F, R477N, R477E, R477Y, Y478W, Y478I, Y478M, M479T, M479H, M479Y, M479F, M479W, I480V, I480L, V481C, V481I, V481T, T484L, E486D, K489I, S490R, S490C, S490V, Q491G, Q491R, Q491N, K493F, K493H, K493Y, K493L, Q494N, A496F, A496S, A496Y, M503L, M503V, M503I, G506R, G506RHPD (“RHPD” disclosed as SEQ ID NO: 69), d507, d508, A508P, d509, Q509D, T513A, T513V, T513C, I518V, Q519P, V521E, V521P, K523Q, K523A, K523R, K523M, K523Y, K523D, G524S, G524A, N525S, N525T, N525D, N525G, N525E, A527P, A527T, A527V, P528A, P528V, R529V, R529A, Y539L, Y539V, D540T, V542E, V542D, T546A, T546K, Q551K, Q551R, P553S, K554S, K554P, K554H, Y555F, Q557K, H559P, A560G, I562V, d562, V564F, I565V, I565F, I565FV, D566E, D566VE, A583Q, A583E, and any combination thereof.

In some embodiments, an engineered ACL described herein includes one or more amino acid alterations selected from the group consisting of: N7K, N7S, A8S, I10W, I10Y, I10F, N12E, N12P, N12D, V15I, D16E, Y19F, A52R, A52K, M54L, L64A, S66R, S67A, R73L, H74K, S82T, L87I, F90L, F90M, V94T, V100L, I11V, S115N, G117S, S119T, Q121D, V129I, T131G, A138D, A138S, K139E, A140K, A140E, A140D, V144T, K150L, K150I, A165S, L166F, M172L, M172A, M172D, R182Q, S191A, G199A, S209P, V212C, S217R, S221T, S221A, S223N, S223I, S223A, S223L, S223H, E224Q, N227E, N227T, N227A, A229I, A229L, A229M, S230Q, S230A, E231H, E231Q, E231P, S232Y, S232K, S232Q, S232N, L233W, L233Y, A234Y, A234F, A234H, T235S, Q238D, Q238H, Q238T, A240T, A240H, A240C, A240S, I243C, I243M, L249M, L249V, S253H, S253M, S253Q, K254M, L256I, L256V, G258T, G258H, G258A, G258L, G258M, N259A, A262K, A262C, A262N, Y268F, G271R, G271Q, I275E, V277L, V277M, Q281V, S288V, S288I, S288F, S288T, S288H, S288Y, T290V, T290A, I296L, I296M, V297I, T300A, I304E, E306D, M316L, A318S, A318R, A318H, A318W, A318Y, A318M, A318T, A318C, A318Q, A318V, A318I, A318L, L320M, L320F, L320T, L320Y, L320W, S321C, A323G, A328V, A328H, A328R, V330I, V330L, T331I, T336K, R352K, I353V, I353L, S363G, P364S, V365C, V365L, V365I, V365M, A366V, F369H, F369S, F369T, F369M, E372S, E372K, E372H, E372N, K374Q, K374R, K374T, K374P, K374H, K374S, K374A, K374D, K374E, R376T, I378A, I378P, I378S, I378V, S388T, V390C, V390A, N394D, V398N, D406L, P407D, Q416N, Q416E, M432F, E443R, E443K, C445A, D455S, D455G, H462R, V469M, A470G, L471R, L471M, L471W, I480V, V481I, V481C, T484L, E486D, S490C, Q491R, Q491G, K493L, K493H, K493Y, Q494N, A496Y, A508P, T513A, T513V, T513C, I518V, Q519P, V521E, V521P, K523R, K523Q, K523A, K523Y, K523M, K523D, N525D, N525S, N525T, N525G, N525E, T546K, K554H, K554S, K554P, Y555F, A560G, I565V, A583Q, A583E and any combination thereof.

In some embodiments, an engineered ACL described herein includes one or more amino acid alterations selected from the group consisting of: I10F, G199A, S223L, S223H, S223A, A229M, S230Q, S230A, E231P, S232N, L233W, L233Y, A234H, T235S, Q238T, I243C, I243M, L256I, L256V, G258L, G258M, V277M, S288Y, S288H, T300A, A318V, A318T, A318C, A318I, A318L, A318Q, A318M, A318Y, L320W, L320Y, S321C, A323G, A328R, V330L, P364S, V365I, V365M, F369M, E372N, K374E, I378V, H462R, V469M, I480V, V481C, T484L, T513C, T513A, T513V, K523D, N525G, N525E, N525T, K554S, K554P, A560G, A583E and any combination thereof.

In some embodiments, an engineered ACL described herein includes one or more amino acid alterations selected from the group consisting of: L233W, P364S, S232N, L320W, A318V, S321C, S223L, L320Y, T513C, Q238T, L233Y, K523D, A318T, S288Y, A318C, A234H, A323G, T513A, S288H, S223H, A318I, A318L, G258L and any combination thereof.

In some embodiments, an engineered ACL described herein includes a single alteration or a combination of alterations described in TABLE 8.

In some embodiments, an engineered ACL described herein includes one or more amino acid alterations at a position corresponding to position 5, 6, 7, 8, 10, 12, 13, 14, 15, 16, 19, 20, 22, 25, 28, 37, 39, 40, 41, 43, 44, 45, 48, 50, 51, 53, 57, 58, 59, 61, 62, 63, 66, 67, 69, 71, 72, 74, 75, 76, 77, 78, 80, 81, 83, 84, 85, 86, 87, 88, 92, 93, 94, 95, 97, 98, 99, 100, 101, 103, 104, 106, 107, 108, 109, 110, 113, 114, 115, 116, 119, 120, 121, 122, 123, 124, 125, 126, 127, 128, 130, 131, 136, 137, 140, 141, 142, 145, 147, 148, 149, 150, 151, 157, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 216, 218, 219, 220, 221, 222, 224, 225, 226, 227, 228, 230, 231, 232, 233, 234, 235, 236, 237, 238, 239, 240, 241, 242, 243, 244, 245, 246, 247, 251, 252, 253, 254, 255, 256, 257, 259, 260, 262, 263, 264, 265, 266, 267, 268, 269, 270, 271, 272, 273, 274, 275, 277, 278, 279, 281, 282, 283, 284, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 341, 342, 343, 344, 345, 346, 347, 348, 349, 351, 352, 353, 354, 355, 357, 358, 359, 361, 362, 363, 365, 366, 367, 368, 369, 370, 372, 373, 375, 376, 377, 378, 379, 380, 381, 382, 383, 384, 385, 386, 387, 388, 389, 391, 392, 394, 395, 396, 398, 399, 400, 401, 402, 404, 405, 406, 407, 409, 411, 413, 414, 415, 416, 417, 418, 419, 420, 421, 425, 430, 432, 433, 434, 435, 441, 442, 443, 446, 447, 449, 450, 451, 452, 453, 455, 456, 458, 459, 460, 461, 462, 463, 464, 465, 466, 467, 468, 469, 470, 471, 473, 474, 476, 478, 479, 480, 481, 483, 485, 490, 492, 493, 496, 497, 498, 502, 504, 507, 508, 510, 511, 512, 513, 514, 515, 516, 518, 519, 521, 526, 527, 532, 535, 540, 542, 546, 552, 554, 558, 560, 561, 563, 564, 565, 566, 567, 568, 569, 570, 572, 573, 574, 575, 577, 578, 583, 584, 585, 587, 588, 591, 593, 595, 596, 598, 599, 601, or a combination thereof, in SEQ ID NO: 2.

In some embodiments, an engineered ACL described herein includes one or more amino acid alterations at a position corresponding to position 5, 6, 7, 8, 10, 12, 13, 14, 15, 16, 19, 20, 22, 25, 28, 37, 39, 40, 41, 43, 45, 50, 51, 53, 57, 58, 59, 61, 62, 63, 67, 69, 71, 72, 74, 75, 76, 77, 78, 80, 81, 83, 84, 85, 86, 87, 88, 92, 93, 94, 95, 97, 98, 99, 100, 101, 103, 104, 106, 108, 109, 110, 113, 116, 119, 120, 121, 123, 124, 125, 126, 127, 128, 130, 131, 136, 137, 140, 141, 142, 145, 147, 148, 149, 150, 151, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 218, 219, 220, 221, 222, 224, 226, 227, 228, 230, 232, 233, 234, 235, 236, 237, 238, 239, 240, 241, 242, 243, 244, 245, 246, 247, 252, 253, 254, 255, 256, 260, 262, 263, 264, 265, 266, 267, 268, 270, 271, 272, 273, 274, 275, 277, 278, 279, 281, 283, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 342, 343, 344, 345, 346, 347, 348, 349, 351, 352, 353, 354, 355, 357, 358, 359, 362, 363, 365, 366, 367, 368, 369, 370, 375, 376, 377, 378, 379, 380, 381, 382, 383, 384, 385, 386, 387, 388, 389, 391, 392, 394, 396, 398, 399, 400, 401, 404, 406, 407, 409, 411, 414, 416, 419, 420, 421, 425, 430, 432, 433, 434, 435, 441, 442, 443, 446, 447, 450, 451, 452, 453, 456, 460, 461, 462, 463, 465, 466, 467, 468, 469, 470, 471, 473, 476, 478, 479, 480, 481, 485, 490, 492, 498, 507, 508, 510, 511, 512, 514, 515, 516, 518, 519, 521, 526, 527, 532, 535, 540, 542, 546, 552, 554, 560, 561, 563, 564, 565, 566, 567, 568, 569, 570, 572, 573, 574, 575, 577, 578, 583, 584, 585, 587, 588, 591, 593, 595, 596, 598, 599, 601 or a combination thereof, in SEQ ID NO: 2.

In some embodiments, an engineered ACL of described herein includes one or more amino acid alterations at a position corresponding to 5, 6, 7, 8, 10, 12, 14, 15, 16, 19, 20, 22, 28, 39, 41, 43, 45, 50, 57, 58, 61, 62, 63, 67, 69, 71, 72, 76, 77, 78, 81, 84, 85, 86, 87, 92, 93, 94, 95, 98, 104, 108, 109, 113, 116, 119, 120, 123, 124,125, 126, 127, 130, 131, 136, 140, 141, 142, 145, 147, 148, 149, 151, 159, 161, 163, 164, 167, 168, 169, 170, 173, 175, 176, 177, 181, 183, 185, 186, 187, 190, 191, 193, 194, 195, 201, 205, 209, 214, 218, 219, 220, 221, 226, 228, 230, 232, 234, 237, 238, 239, 241, 242, 243, 244, 246, 247, 253, 254, 260, 262, 263, 266, 267, 272, 273, 274, 278, 279, 281, 283, 285, 290, 291, 292, 295, 296, 298, 300, 302, 303, 304, 305, 307, 311, 313, 314, 315, 316, 318, 321, 322, 330, 333, 334, 336, 337, 338, 339, 340, 344, 345, 346, 347, 348, 349, 351, 352, 353, 354, 355, 357, 358, 359, 362, 363, 365, 367, 369, 375, 376, 379, 382, 383, 384, 386, 387, 388, 389, 399, 400, 401, 404, 407, 409, 416, 419, 420, 421, 430, 432, 433, 434, 441, 442, 446, 450, 452, 453, 456, 460, 461, 465, 466, 471, 473, 476, 479, 480, 481, 485, 490, 492, 507, 511, 514, 515, 516, 518, 519, 521, 526, 527, 532, 540, 542, 546, 554, 561, 564, 565, 566, 567, 568, 570, 572, 574, 575, 577, 584, 587, 595, 596, 599 or a combination thereof, in SEQ ID NO: 2.

In some embodiments, an engineered ACL described herein includes one or more amino acid alterations at a position corresponding to 5, 6, 7, 8, 10, 12, 14, 15, 19, 20, 22, 41, 45, 50, 58, 62, 63, 67, 69, 72, 76, 77, 81, 86, 87, 92, 93, 94, 95, 98, 104, 108, 113, 116, 120, 123, 124, 125, 126, 127, 130, 136, 140, 141, 142, 145, 147, 148, 149, 151, 159, 161, 163, 164, 167, 168, 169, 175, 176, 177, 185, 186, 187, 190, 191, 193, 195, 201, 205, 209, 214, 219, 220, 221, 228, 230, 232, 234, 237, 238, 239, 241, 242, 243, 244, 246, 247, 253, 254, 260, 263, 266, 267, 272, 273, 274, 278, 279, 281, 283, 291, 292, 295, 298, 300, 302, 303, 304, 305, 307, 311, 313, 314, 315, 318, 321, 322, 330, 333, 334, 336, 337, 338, 340, 344, 348, 349, 351, 353, 354, 355, 357, 358, 359, 362, 363, 365, 367, 369, 376, 379, 382, 383, 386, 387, 388, 389, 399, 400, 401, 404, 407, 409, 420, 421, 430, 432, 433, 434, 442, 446, 450, 452, 453, 456, 460, 461, 465, 466, 473, 476, 479, 480, 481, 485, 492, 507, 514, 515, 516, 518, 519, 521, 526, 527, 532, 540, 542, 554, 564, 565, 567, 568, 570, 572, 575, 584, 587, 595, 596 or a combination thereof, in SEQ ID NO: 2.

In some embodiments, an engineered ACL having one or more amino acid alterations results in an engineered ACL comprising: (a) R, E at a residue corresponding to position 5 in SEQ ID NO: 2; (b) L at a residue corresponding to position 6 in SEQ ID NO: 2; (c) K, T at a residue corresponding to position 7 in SEQ ID NO: 2; (d) P at a residue corresponding to position 8 in SEQ ID NO: 2; (e) K, D, P, N, R at a residue corresponding to position 10 in SEQ ID NO: 2; (f) A, I, V, M at a residue corresponding to position 12 in SEQ ID NO: 2; (g) C at a residue corresponding to position 13 in SEQ ID NO: 2; (h) P, A, Q at a residue corresponding to position 14 in SEQ ID NO: 2; (i) E, L, S, R at a residue corresponding to position 15 in SEQ ID NO: 2; (j) C, L, I at a residue corresponding to position 16 in SEQ ID NO: 2; (k) D, R at a residue corresponding to position 19 in SEQ ID NO: 2; (1) H, K, R, D at a residue corresponding to position 20 in SEQ ID NO: 2; (m) D, E at a residue corresponding to position 22 in SEQ ID NO: 2; (n) N at a residue corresponding to position 25 in SEQ ID NO: 2; (o) M at a residue corresponding to position 28 in SEQ ID NO: 2; (p) C at a residue corresponding to position 37 in SEQ ID NO: 2; (q) R at a residue corresponding to position 39 in SEQ ID NO: 2; (r) T at a residue corresponding to position 40 in SEQ ID NO: 2; (s) M, L, V, I at a residue corresponding to position 41 in SEQ ID NO: 2; (t) S at a residue corresponding to position 43 in SEQ ID NO: 2; (u) M at a residue corresponding to position 44 in SEQ ID NO: 2; (v) I, V at a residue corresponding to position 45 in SEQ ID NO: 2; (w) P at a residue corresponding to position 48 in SEQ ID NO: 2; (x) I, F, R, V, L at a residue corresponding to position 50 in SEQ ID NO: 2; (y) R at a residue corresponding to position 51 in SEQ ID NO: 2; (z) L at a residue corresponding to position 53 in SEQ ID NO: 2; (aa) F at a residue corresponding to position 57 in SEQ ID NO: 2; (bb) V, L, M, F at a residue corresponding to position 58 in SEQ ID NO: 2; (cc) V at a residue corresponding to position 59 in SEQ ID NO: 2; (dd) P, E at a residue corresponding to position 61 in SEQ ID NO: 2; (ee) D at a residue corresponding to position 62 in SEQ ID NO: 2; (ff) D at a residue corresponding to position 63 in SEQ ID NO: 2; (gg) G at a residue corresponding to position 66 in SEQ ID NO: 2; (hh) K at a residue corresponding to position 67 in SEQ ID NO: 2; (ii) F, Y, L at a residue corresponding to position 69 in SEQ ID NO: 2; (jj) F, W at a residue corresponding to position 71 in SEQ ID NO: 2; (kk) A, R at a residue corresponding to position 72 in SEQ ID NO: 2; (ll) F at a residue corresponding to position 74 in SEQ ID NO: 2; (mm) D at a residue corresponding to position 75 in SEQ ID NO: 2; (nn) T, D, A, R at a residue corresponding to position 76 in SEQ ID NO: 2; (oo) R at a residue corresponding to position 77 in SEQ ID NO: 2; (pp) A at a residue corresponding to position 78 in SEQ ID NO: 2; (qq) K at a residue corresponding to position 80 in SEQ ID NO: 2; (rr) C at a residue corresponding to position 81 in SEQ ID NO: 2; (ss) S, H, Q, N at a residue corresponding to position 83 in SEQ ID NO: 2; (tt) Y, H at a residue corresponding to position 84 in SEQ ID NO: 2; (uu) F at a residue corresponding to position 85 in SEQ ID NO: 2; (vv) A, Q at a residue corresponding to position 86 in SEQ ID NO: 2; (ww) R at a residue corresponding to position 87 in SEQ ID NO: 2; (xx) L at a residue corresponding to position 88 in SEQ ID NO: 2; (yy) K at a residue corresponding to position 92 in SEQ ID NO: 2; (zz) G at a residue corresponding to position 93 in SEQ ID NO: 2; (aaa) R at a residue corresponding to position 94 in SEQ ID NO: 2; (bbb) R at a residue corresponding to position 95 in SEQ ID NO: 2; (ccc) A at a residue corresponding to position 97 in SEQ ID NO: 2; (ddd) M at a residue corresponding to position 98 in SEQ ID NO: 2; (eee) L at a residue corresponding to position 99 in SEQ ID NO: 2; (fff) C at a residue corresponding to position 100 in SEQ ID NO: 2; (ggg) P at a residue corresponding to position 101 in SEQ ID NO: 2; (hhh) R at a residue corresponding to position 103 in SEQ ID NO: 2; (iii) L, W, A at a residue corresponding to position 104 in SEQ ID NO: 2; (jjj) W, F at a residue corresponding to position 106 in SEQ ID NO: 2; (kkk) V, P, I, L, W, F at a residue corresponding to position 107 in SEQ ID NO: 2; (lll) V at a residue corresponding to position 108 in SEQ ID NO: 2; (mmm) V at a residue corresponding to position 109 in SEQ ID NO: 2; (nnn) M, F, Y, A, D at a residue corresponding to position 110 in SEQ ID NO: 2; (ooo) T, C, V at a residue corresponding to position 113 in SEQ ID NO: 2; (ppp) M, W, L, G, A at a residue corresponding to position 114 in SEQ ID NO: 2; (qqq) A at a residue corresponding to position 115 in SEQ ID NO: 2; (rrr) I at a residue corresponding to position 116 in SEQ ID NO: 2; (sss) V, T, I at a residue corresponding to position 119 in SEQ ID NO: 2; (ttt) M, F, V, A, L, W, Y at a residue corresponding to position 120 in SEQ ID NO: 2; (uuu) T at a residue corresponding to position 121 in SEQ ID NO: 2; (vvv) A at a residue corresponding to position 122 in SEQ ID NO: 2; (www) M, V, I at a residue corresponding to position 123 in SEQ ID NO: 2; (xxx) Y, S, N at a residue corresponding to position 124 in SEQ ID NO: 2; (yyy) P, T, W at a residue corresponding to position 125 in SEQ ID NO: 2; (zzz) M, N, R, A at a residue corresponding to position 126 in SEQ ID NO: 2; (aaaa) F, W, L at a residue corresponding to position 127 in SEQ ID NO: 2; (bbbb) R at a residue corresponding to position 128 in SEQ ID NO: 2; (cccc) M at a residue corresponding to position 130 in SEQ ID NO: 2; (dddd) E at a residue corresponding to position 131 in SEQ ID NO: 2; (eeee) E, Q, K at a residue corresponding to position 136 in SEQ ID NO: 2; (ffff) M at a residue corresponding to position 137 in SEQ ID NO: 2; (gggg) M, C, I at a residue corresponding to position 140 in SEQ ID NO: 2; (hhhh) M at a residue corresponding to position 141 in SEQ ID NO: 2; (iiii) N, Q at a residue corresponding to position 142 in SEQ ID NO: 2; (jjjj) G, R, D at a residue corresponding to position 145 in SEQ ID NO: 2; (kkkk) S at a residue corresponding to position 147 in SEQ ID NO: 2; (llll) L, V, A at a residue corresponding to position 148 in SEQ ID NO: 2; (mmmm) V, L at a residue corresponding to position 149 in SEQ ID NO: 2; (nnnn) V at a residue corresponding to position 150 in SEQ ID NO: 2; (oooo) C at a residue corresponding to position 151 in SEQ ID NO: 2; (pppp) L at a residue corresponding to position 157 in SEQ ID NO: 2; (qqqq) R, E at a residue corresponding to position 159 in SEQ ID NO: 2; (rrrr) I at a residue corresponding to position 161 in SEQ ID NO: 2; (ssss) V at a residue corresponding to position 163 in SEQ ID NO: 2; (tttt) A at a residue corresponding to position 164 in SEQ ID NO: 2; (uuuu) R at a residue corresponding to position 165 in SEQ ID NO: 2; (vvvv) R at a residue corresponding to position 167 in SEQ ID NO: 2; (wwww) H at a residue corresponding to position 168 in SEQ ID NO: 2; (xxxx) I at a residue corresponding to position 169 in SEQ ID NO: 2; (yyyy) I, V, L at a residue corresponding to position 170 in SEQ ID NO: 2; (zzzz) V at a residue corresponding to position 173 in SEQ ID NO: 2; (aaaaa) R at a residue corresponding to position 175 in SEQ ID NO: 2; (bbbbb) D at a residue corresponding to position 176 in SEQ ID NO: 2; (ccccc) E at a residue corresponding to position 177 in SEQ ID NO: 2; (ddddd) E at a residue corresponding to position 181 in SEQ ID NO: 2; (eeeee) I at a residue corresponding to position 183 in SEQ ID NO: 2; (fffff) S at a residue corresponding to position 185 in SEQ ID NO: 2; (ggggg) Y at a residue corresponding to position 186 in SEQ ID NO: 2; (hhhhh) D at a residue corresponding to position 187 in SEQ ID NO: 2; (iiiii) V at a residue corresponding to position 189 in SEQ ID NO: 2; (jjjjj) V at a residue corresponding to position 190 in SEQ ID NO: 2; (kkkkk) A, E at a residue corresponding to position 191 in SEQ ID NO: 2; (lllll) T at a residue corresponding to position 192 in SEQ ID NO: 2; (mmmmm) A at a residue corresponding to position 193 in SEQ ID NO: 2; (nnnnn) S at a residue corresponding to position 194 in SEQ ID NO: 2; (ooooo) S at a residue corresponding to position 195 in SEQ ID NO: 2; (ppppp) P at a residue corresponding to position 196 in SEQ ID NO: 2; (qqqqq) A, P, L at a residue corresponding to position 201 in SEQ ID NO: 2; (rrrrr) T at a residue corresponding to position 205 in SEQ ID NO: 2; (sssss) D at a residue corresponding to position 208 in SEQ ID NO: 2; (ttttt) I, T, P, V at a residue corresponding to position 209 in SEQ ID NO: 2; (uuuuu) L, T, C, M, V, A at a residue corresponding to position 211 in SEQ ID NO: 2; (vvvvv) L, M at a residue corresponding to position 212 in SEQ ID NO: 2; (wwwww) I, C, V, Q, M at a residue corresponding to position 213 in SEQ ID NO: 2; (xxxxx) Y at a residue corresponding to position 214 in SEQ ID NO: 2; (yyyyy) S at a residue corresponding to position 215 in SEQ ID NO: 2; (zzzzz) G at a residue corresponding to position 216 in SEQ ID NO: 2; (aaaaaa) T at a residue corresponding to position 218 in SEQ ID NO: 2; (bbbbbb) T at a residue corresponding to position 219 in SEQ ID NO: 2; (cccccc) G at a residue corresponding to position 220 in SEQ ID NO: 2; (dddddd) T, M, L, R, K, H at a residue corresponding to position 221 in SEQ ID NO: 2; (eeeeee) S at a residue corresponding to position 222 in SEQ ID NO: 2; (ffffff) A at a residue corresponding to position 224 in SEQ ID NO: 2; (gggggg) A at a residue corresponding to position 225 in SEQ ID NO: 2; (hhhhhh) M, I, V at a residue corresponding to position 226 in SEQ ID NO: 2; (iiiiii) L, D, H, I at a residue corresponding to position 227 in SEQ ID NO: 2; (jjjjjj) S at a residue corresponding to position 228 in SEQ ID NO: 2; (kkkkkk) H, K at a residue corresponding to position 230 in SEQ ID NO: 2; (llllll) N at a residue corresponding to position 231 in SEQ ID NO: 2; (mmmmmm) L, I, V at a residue corresponding to position 232 in SEQ ID NO: 2; (nnnnnn) L, V, M, W, A, I at a residue corresponding to position 233 in SEQ ID NO: 2; (oooooo) S, W at a residue corresponding to position 234 in SEQ ID NO: 2; (pppppp) Q, S at a residue corresponding to position 235 in SEQ ID NO: 2; (qqqqqq) A, C, I, M, V at a residue corresponding to position 236 in SEQ ID NO: 2; (rrrrrr) A, M, Q, R, E at a residue corresponding to position 237 in SEQ ID NO: 2; (ssssss) M, S, W, N, H at a residue corresponding to position 238 in SEQ ID NO: 2; (tttttt) F, M, V, C, L at a residue corresponding to position 239 in SEQ ID NO: 2; (uuuuuu) A, R at a residue corresponding to position 240 in SEQ ID NO: 2; (vvvvvv) A, H, E, Q at a residue corresponding to position 241 in SEQ ID NO: 2; (wwwwww) V, C, T, W at a residue corresponding to position 242 in SEQ ID NO: 2; (xxxxxx) I, F, M, W, V at a residue corresponding to position 243 in SEQ ID NO: 2; (yyyyyy) E, P, Q, H, G at a residue corresponding to position 244 in SEQ ID NO: 2; (zzzzzz) M, L, H, W, Y at a residue corresponding to position 245 in SEQ ID NO: 2; (aaaaaaa) I, T, M at a residue corresponding to position 246 in SEQ ID NO: 2; (bbbbbbb) R, P, E at a residue corresponding to position 247 in SEQ ID NO: 2; (ccccccc) E at a residue corresponding to position 251 in SEQ ID NO: 2; (ddddddd) H, V at a residue corresponding to position 252 in SEQ ID NO: 2; (eeeeeee) Y, M, F, L, V at a residue corresponding to position 253 in SEQ ID NO: 2; (fffffff) I, F, V, M at a residue corresponding to position 254 in SEQ ID NO: 2; (ggggggg) A, T, N, L, I, G, H, C, V, S at a residue corresponding to position 255 in SEQ ID NO: 2; (hhhhhhh) F, V, I, C, Y at a residue corresponding to position 256 in SEQ ID NO: 2; (iiiiiii) M at a residue corresponding to position 257 in SEQ ID NO: 2; (jjjjjjj) L, F at a residue corresponding to position 259 in SEQ ID NO: 2; (kkkkkkk) H, S, Y, F at a residue corresponding to position 260 in SEQ ID NO: 2; (lllllll) C, T, A, I, M at a residue corresponding to position 262 in SEQ ID NO: 2; (mmmmmmm) A, Y, H at a residue corresponding to position 263 in SEQ ID NO: 2; (nnnnnnn) A at a residue corresponding to position 264 in SEQ ID NO: 2; (ooooooo) M, L, W, F, C at a residue corresponding to position 265 in SEQ ID NO: 2; (ppppppp) N, T, M, V at a residue corresponding to position 266 in SEQ ID NO: 2; (qqqqqqq) I, G, M, W, F, L, V, C, A at a residue corresponding to position 267 in SEQ ID NO: 2; (rrrrrrr) M, A, C, G, V at a residue corresponding to position 268 in SEQ ID NO: 2; (sssssss) L, M, I, V at a residue corresponding to position 269 in SEQ ID NO: 2; (ttttttt) M, H, Y, T at a residue corresponding to position 270 in SEQ ID NO: 2; (uuuuuuu) A, P, C, T, L at a residue corresponding to position 271 in SEQ ID NO: 2; (vvvvvvv) C, V, P, A, T at a residue corresponding to position 272 in SEQ ID NO: 2; (wwwwwww) M, V, F, I at a residue corresponding to position 273 in SEQ ID NO: 2; (xxxxxxx) Y, R, V, M, C, H, W at a residue corresponding to position 274 in SEQ ID NO: 2; (yyyyyyy) G, C, S, H at a residue corresponding to position 275 in SEQ ID NO: 2; (zzzzzzz) A, C at a residue corresponding to position 277 in SEQ ID NO: 2; (aaaaaaaa) C, T, A, H at a residue corresponding to position 278 in SEQ ID NO: 2; (bbbbbbbb) V, M, I at a residue corresponding to position 279 in SEQ ID NO: 2; (cccccccc) F, L, V, M at a residue corresponding to position 281 in SEQ ID NO: 2; (dddddddd) P, M, E, A, H at a residue corresponding to position 282 in SEQ ID NO: 2; (eeeeeeee) E, D, P, R at a residue corresponding to position 283 in SEQ ID NO: 2; (ffffffff) F, R, W at a residue corresponding to position 284 in SEQ ID NO: 2; (gggggggg) R, F, W, D at a residue corresponding to position 285 in SEQ ID NO: 2; (hhhhhhhh) D, F at a residue corresponding to position 286 in SEQ ID NO: 2; (iiiiiiii) P, A, V at a residue corresponding to position 287 in SEQ ID NO: 2; (jjjjjjjj) A at a residue corresponding to position 290 in SEQ ID NO: 2; (kkkkkkkk) M, V, L, P, I at a residue corresponding to position 291 in SEQ ID NO: 2; (llllllll) H at a residue corresponding to position 292 in SEQ ID NO: 2; (mmmmmmmm) I, L, A at a residue corresponding to position 294 in SEQ ID NO: 2; (nnnnnnnn) W, M, V, I at a residue corresponding to position 295 in SEQ ID NO: 2; (oooooooo) D, Q at a residue corresponding to position 296 in SEQ ID NO: 2; (pppppppp) M, V, L at a residue corresponding to position 298 in SEQ ID NO: 2; (qqqqqqqq) A at a residue corresponding to position 299 in SEQ ID NO: 2; (rrrrrrrr) Q at a residue corresponding to position 300 in SEQ ID NO: 2; (ssssssss) Q at a residue corresponding to position 302 in SEQ ID NO: 2; (tttttttt) V, I, A, P, C at a residue corresponding to position 303 in SEQ ID NO: 2; (uuuuuuuu) S at a residue corresponding to position 304 in SEQ ID NO: 2; (vvvvvvvv) W, Y, V, I, F, H, M at a residue corresponding to position 305 in SEQ ID NO: 2; (wwwwwwww) V, Y, I, M, L, F at a residue corresponding to position 306 in SEQ ID NO: 2; (xxxxxxxx) P, W, Y, H, M at a residue corresponding to position 307 in SEQ ID NO: 2; (yyyyyyyy) T, A, S, L, G, V, M at a residue corresponding to position 308 in SEQ ID NO: 2; (zzzzzzzz) A, R, P, T at a residue corresponding to position 311 in SEQ ID NO: 2; (aaaaaaaaa) I, M, V, F at a residue corresponding to position 312 in SEQ ID NO: 2; (bbbbbbbbb) L, F, W at a residue corresponding to position 313 in SEQ ID NO: 2; (ccccccccc) Q, E, H at a residue corresponding to position 314 in SEQ ID NO: 2; (ddddddddd) R, A, K, L at a residue corresponding to position 315 in SEQ ID NO: 2; (eeeeeeeee) W, M, I, F at a residue corresponding to position 316 in SEQ ID NO: 2; (fffffffff) H, D at a residue corresponding to position 318 in SEQ ID NO: 2; (ggggggggg) R, Q at a residue corresponding to position 321 in SEQ ID NO: 2; (hhhhhhhhh) Q, K, A, H at a residue corresponding to position 322 in SEQ ID NO: 2; (iiiiiiiii) L, F at a residue corresponding to position 330 in SEQ ID NO: 2; (jjjjjjjjj) G at a residue corresponding to position 331 in SEQ ID NO: 2; (kkkkkkkkk) S at a residue corresponding to position 333 in SEQ ID NO: 2; (lllllllll) I, M, V at a residue corresponding to position 334 in SEQ ID NO: 2; (mmmmmmmmm) R, K at a residue corresponding to position 335 in SEQ ID NO: 2; (nnnnnnnnn) T, I, M, Y, V, F, W at a residue corresponding to position 336 in SEQ ID NO: 2; (ooooooooo) L, M, F, A, V at a residue corresponding to position 337 in SEQ ID NO: 2; (ppppppppp) W, L, M, V at a residue corresponding to position 338 in SEQ ID NO: 2; (qqqqqqqqq) F, T, S, C at a residue corresponding to position 339 in SEQ ID NO: 2; (rrrrrrrrr) G at a residue corresponding to position 340 in SEQ ID NO: 2; (sssssssss) A, S at a residue corresponding to position 341 in SEQ ID NO: 2; (ttttttttt) S at a residue corresponding to position 342 in SEQ ID NO: 2; (uuuuuuuuu) P, A at a residue corresponding to position 343 in SEQ ID NO: 2; (vvvvvvvvv) M, I, V, C at a residue corresponding to position 344 in SEQ ID NO: 2; (wwwwwwwww) S at a residue corresponding to position 345 in SEQ ID NO: 2; (xxxxxxxxx) V, A, P, E at a residue corresponding to position 346 in SEQ ID NO: 2; (yyyyyyyyy) E at a residue corresponding to position 347 in SEQ ID NO: 2; (zzzzzzzzz) T, I, L at a residue corresponding to position 348 in SEQ ID NO: 2; (aaaaaaaaaa) M, F, L, A, I, Y at a residue corresponding to position 349 in SEQ ID NO: 2; (bbbbbbbbbb) F, R, Q, W at a residue corresponding to position 351 in SEQ ID NO: 2; (cccccccccc) I, M, Y at a residue corresponding to position 352 in SEQ ID NO: 2; (dddddddddd) R at a residue corresponding to position 353 in SEQ ID NO: 2; (eeeeeeeeee) K at a residue corresponding to position 354 in SEQ ID NO: 2; (ffffffffff) A, R at a residue corresponding to position 355 in SEQ ID NO: 2; (gggggggggg) W, G, V, L, P, I, F, A at a residue corresponding to position 357 in SEQ ID NO: 2; (hhhhhhhhhh) Q at a residue corresponding to position 358 in SEQ ID NO: 2; (iiiiiiiiii) H, Q at a residue corresponding to position 359 in SEQ ID NO: 2; (jjjjjjjjjj) F at a residue corresponding to position 361 in SEQ ID NO: 2; (kkkkkkkkkk) H, L, C at a residue corresponding to position 362 in SEQ ID NO: 2; (llllllllll) D, V, I, G at a residue corresponding to position 363 in SEQ ID NO: 2; (mmmmmmmmmm) M, V, I at a residue corresponding to position 365 in SEQ ID NO: 2; (nnnnnnnnnn) I, V, L, F at a residue corresponding to position 366 in SEQ ID NO: 2; (oooooooooo) T, C, M, I, V, L at a residue corresponding to position 367 in SEQ ID NO: 2; (pppppppppp) N, Q, E, T at a residue corresponding to position 368 in SEQ ID NO: 2; (qqqqqqqqqq) G, A at a residue corresponding to position 369 in SEQ ID NO: 2; (rrrrrrrrrr) Y, F at a residue corresponding to position 370 in SEQ ID NO: 2; (ssssssssss) L, Q at a residue corresponding to position 372 in SEQ ID NO: 2; (tttttttttt) S at a residue corresponding to position 373 in SEQ ID NO: 2; (uuuuuuuuuu) A, C at a residue corresponding to position 375 in SEQ ID NO: 2; (vvvvvvvvvv) T, S, C, G at a residue corresponding to position 376 in SEQ ID NO: 2; (wwwwwwwwww) G, M at a residue corresponding to position 377 in SEQ ID NO: 2; (xxxxxxxxxx) C, T, V at a residue corresponding to position 378 in SEQ ID NO: 2; (yyyyyyyyyy) C, V, I, L, A at a residue corresponding to position 379 in SEQ ID NO: 2; (zzzzzzzzzz) F, C, H, S, M, T, V at a residue corresponding to position 380 in SEQ ID NO: 2; (aaaaaaaaaaa) N, F, L, C, V at a residue corresponding to position 381 in SEQ ID NO: 2; (bbbbbbbbbbb) L, N, C, T, P at a residue corresponding to position 382 in SEQ ID NO: 2; (ccccccccccc) D, N, R, P, Y at a residue corresponding to position 383 in SEQ ID NO: 2; (ddddddddddd) P, N, D, W at a residue corresponding to position 384 in SEQ ID NO: 2; (eeeeeeeeeee) G, D at a residue corresponding to position 385 in SEQ ID NO: 2; (fffffffffff) D, V, L, T at a residue corresponding to position 386 in SEQ ID NO: 2; (ggggggggggg) D, R, N, H, E at a residue corresponding to position 387 in SEQ ID NO: 2; (hhhhhhhhhhh) K, H at a residue corresponding to position 388 in SEQ ID NO: 2; (iiiiiiiiiii) A, P, L at a residue corresponding to position 389 in SEQ ID NO: 2; (jjjjjjjjjjj) S, T at a residue corresponding to position 391 in SEQ ID NO: 2; (kkkkkkkkkkk) V, C at a residue corresponding to position 392 in SEQ ID NO: 2; (lllllllllll) R, K, I, Q, L at a residue corresponding to position 394 in SEQ ID NO: 2; (mmmmmmmmmmm) A at a residue corresponding to position 395 in SEQ ID NO: 2; (nnnnnnnnnnn) F, I, M, V at a residue corresponding to position 396 in SEQ ID NO: 2; (ooooooooooo) D, H, N at a residue corresponding to position 398 in SEQ ID NO: 2; (ppppppppppp) M, V, T, C, I at a residue corresponding to position 399 in SEQ ID NO: 2; (qqqqqqqqqqq) D, Q at a residue corresponding to position 400 in SEQ ID NO: 2; (rrrrrrrrrrr) M, I, C, L, A at a residue corresponding to position 401 in SEQ ID NO: 2; (sssssssssss) R at a residue corresponding to position 402 in SEQ ID NO: 2; (ttttttttttt) V at a residue corresponding to position 404 in SEQ ID NO: 2; (uuuuuuuuuuu) D at a residue corresponding to position 405 in SEQ ID NO: 2; (vvvvvvvvvvv) E at a residue corresponding to position 406 in SEQ ID NO: 2; (wwwwwwwwwww) P at a residue corresponding to position 407 in SEQ ID NO: 2; (xxxxxxxxxxx) Q, E, G at a residue corresponding to position 409 in SEQ ID NO: 2; (yyyyyyyyyyy) D, S, V, G, N at a residue corresponding to position 411 in SEQ ID NO: 2; (zzzzzzzzzzz) I, V at a residue corresponding to position 413 in SEQ ID NO: 2; (aaaaaaaaaaaa) W, L, V, C, Q at a residue corresponding to position 414 in SEQ ID NO: 2; (bbbbbbbbbbbb) I at a residue corresponding to position 415 in SEQ ID NO: 2; (cccccccccccc) R at a residue corresponding to position 416 in SEQ ID NO: 2; (dddddddddddd) S at a residue corresponding to position 417 in SEQ ID NO: 2; (eeeeeeeeeeee) D at a residue corresponding to position 418 in SEQ ID NO: 2; (ffffffffffff) H, S, Q, M at a residue corresponding to position 419 in SEQ ID NO: 2; (gggggggggggg) V at a residue corresponding to position 420 in SEQ ID NO: 2; (hhhhhhhhhhhh) T, F at a residue corresponding to position 421 in SEQ ID NO: 2; (iiiiiiiiiiii) H, L at a residue corresponding to position 425 in SEQ ID NO: 2; (jjjjjjjjjjjj) A at a residue corresponding to position 430 in SEQ ID NO: 2; (kkkkkkkkkkkk) K at a residue corresponding to position 432 in SEQ ID NO: 2; (llllllllllll) R, D at a residue corresponding to position 433 in SEQ ID NO: 2; (mmmmmmmmmmmm) T, V at a residue corresponding to position 434 in SEQ ID NO: 2; (nnnnnnnnnnnn) I, M, L at a residue corresponding to position 435 in SEQ ID NO: 2; (oooooooooooo) F, M, Y at a residue corresponding to position 441 in SEQ ID NO: 2; (pppppppppppp) K, H, Y, R, C at a residue corresponding to position 442 in SEQ ID NO: 2; (qqqqqqqqqqqq) S at a residue corresponding to position 443 in SEQ ID NO: 2; (rrrrrrrrrrrr) M, I, L, V at a residue corresponding to position 446 in SEQ ID NO: 2; (ssssssssssss) A, G at a residue corresponding to position 447 in SEQ ID NO: 2; (tttttttttttt) I at a residue corresponding to position 449 in SEQ ID NO: 2; (uuuuuuuuuuuu) D, M at a residue corresponding to position 450 in SEQ ID NO: 2; (vvvvvvvvvvvv) E at a residue corresponding to position 451 in SEQ ID NO: 2; (wwwwwwwwwwww) P at a residue corresponding to position 452 in SEQ ID NO: 2; (xxxxxxxxxxxx) G at a residue corresponding to position 453 in SEQ ID NO: 2; (yyyyyyyyyyyy) E, G, R at a residue corresponding to position 455 in SEQ ID NO: 2; (zzzzzzzzzzzz) D, E, Q, N at a residue corresponding to position 456 in SEQ ID NO: 2; (aaaaaaaaaaaaa) N, F, Y, W at a residue corresponding to position 458 in SEQ ID NO: 2; (bbbbbbbbbbbbb) Y at a residue corresponding to position 459 in SEQ ID NO: 2; (ccccccccccccc) Y, F, I, R, W at a residue corresponding to position 460 in SEQ ID NO: 2; (ddddddddddddd) I, L, Y at a residue corresponding to position 461 in SEQ ID NO: 2; (eeeeeeeeeeeee) I, T, V at a residue corresponding to position 462 in SEQ ID NO: 2; (fffffffffffff) D at a residue corresponding to position 463 in SEQ ID NO: 2; (ggggggggggggg) D at a residue corresponding to position 464 in SEQ ID NO: 2; (hhhhhhhhhhhhh) L at a residue corresponding to position 465 in SEQ ID NO: 2; (iiiiiiiiiiiii) D, K, A at a residue corresponding to position 466 in SEQ ID NO: 2; (jjjjjjjjjjjjj) K at a residue corresponding to position 467 in SEQ ID NO: 2; (kkkkkkkkkkkkk) M at a residue corresponding to position 468 in SEQ ID NO: 2; (lllllllllllll) L at a residue corresponding to position 469 in SEQ ID NO: 2; (mmmmmmmmmmmmm) V at a residue corresponding to position 470 in SEQ ID NO: 2; (nnnnnnnnnnnnn) T, V at a residue corresponding to position 471 in SEQ ID NO: 2; (ooooooooooooo) S, N at a residue corresponding to position 473 in SEQ ID NO: 2; (ppppppppppppp) E at a residue corresponding to position 474 in SEQ ID NO: 2; (qqqqqqqqqqqqq) N, Y, L, F, V, I at a residue corresponding to position 476 in SEQ ID NO: 2; (rrrrrrrrrrrrr) T, L at a residue corresponding to position 478 in SEQ ID NO: 2; (sssssssssssss) P, V at a residue corresponding to position 479 in SEQ ID NO: 2; (ttttttttttttt) L, I, S, P, A, V at a residue corresponding to position 480 in SEQ ID NO: 2; (uuuuuuuuuuuuu) P, E at a residue corresponding to position 481 in SEQ ID NO: 2; (vvvvvvvvvvvvv) E at a residue corresponding to position 483 in SEQ ID NO: 2; (wwwwwwwwwwwww) A, L, E, R at a residue corresponding to position 485 in SEQ ID NO: 2; (xxxxxxxxxxxxx) C at a residue corresponding to position 490 in SEQ ID NO: 2; (yyyyyyyyyyyyy) P at a residue corresponding to position 492 in SEQ ID NO: 2; (zzzzzzzzzzzzz) F at a residue corresponding to position 493 in SEQ ID NO: 2; (aaaaaaaaaaaaaa) H at a residue corresponding to position 496 in SEQ ID NO: 2; (bbbbbbbbbbbbbb) A at a residue corresponding to position 497 in SEQ ID NO: 2; (cccccccccccccc) C at a residue corresponding to position 498 in SEQ ID NO: 2; (dddddddddddddd) P at a residue corresponding to position 502 in SEQ ID NO: 2; (eeeeeeeeeeeeee) H at a residue corresponding to position 504 in SEQ ID NO: 2; (ffffffffffffff) Y, V, W at a residue corresponding to position 507 in SEQ ID NO: 2; (gggggggggggggg) V at a residue corresponding to position 508 in SEQ ID NO: 2; (hhhhhhhhhhhhhh) A at a residue corresponding to position 510 in SEQ ID NO: 2; (iiiiiiiiiiiiii) V, L at a residue corresponding to position 511 in SEQ ID NO: 2; (jjjjjjjjjjjjjj) I at a residue corresponding to position 512 in SEQ ID NO: 2; (kkkkkkkkkkkkkk) V at a residue corresponding to position 513 in SEQ ID NO: 2; (llllllllllllll) N at a residue corresponding to position 514 in SEQ ID NO: 2; (mmmmmmmmmmmmmm) D, S at a residue corresponding to position 515 in SEQ ID NO: 2; (nnnnnnnnnnnnnn) A at a residue corresponding to position 516 in SEQ ID NO: 2; (oooooooooooooo) I at a residue corresponding to position 518 in SEQ ID NO: 2; (pppppppppppppp) T at a residue corresponding to position 519 in SEQ ID NO: 2; (qqqqqqqqqqqqqq) P, W at a residue corresponding to position 521 in SEQ ID NO: 2; (rrrrrrrrrrrrrr) L, M at a residue corresponding to position 526 in SEQ ID NO: 2; (ssssssssssssss) L at a residue corresponding to position 527 in SEQ ID NO: 2; (tttttttttttttt) R, H at a residue corresponding to position 532 in SEQ ID NO: 2; (uuuuuuuuuuuuuu) L at a residue corresponding to position 535 in SEQ ID NO: 2; (vvvvvvvvvvvvvv) Q at a residue corresponding to position 540 in SEQ ID NO: 2; (wwwwwwwwwwwwww) Y, W at a residue corresponding to position 542 in SEQ ID NO: 2; (xxxxxxxxxxxxxx) N at a residue corresponding to position 546 in SEQ ID NO: 2; (yyyyyyyyyyyyyy) Q at a residue corresponding to position 552 in SEQ ID NO: 2; (zzzzzzzzzzzzzz) E at a residue corresponding to position 554 in SEQ ID NO: 2; (aaaaaaaaaaaaaaa) F at a residue corresponding to position 558 in SEQ ID NO: 2; (bbbbbbbbbbbbbbb) D at a residue corresponding to position 560 in SEQ ID NO: 2; (ccccccccccccccc) D at a residue corresponding to position 561 in SEQ ID NO: 2; (ddddddddddddddd) F, L, W at a residue corresponding to position 563 in SEQ ID NO: 2; (eeeeeeeeeeeeeee) D, K, S, L, T, R at a residue corresponding to position 564 in SEQ ID NO: 2; (fffffffffffffff) M, I, V, P, T at a residue corresponding to position 565 in SEQ ID NO: 2; (ggggggggggggggg) A, E, D, P at a residue corresponding to position 566 in SEQ ID NO: 2; (hhhhhhhhhhhhhhh) K, Q, T, N, S, A at a residue corresponding to position 567 in SEQ ID NO: 2; (iiiiiiiiiiiiiii) G, N, D at a residue corresponding to position 568 in SEQ ID NO: 2; (jjjjjjjjjjjjjjj) M, K at a residue corresponding to position 569 in SEQ ID NO: 2; (kkkkkkkkkkkkkkk) V, L at a residue corresponding to position 570 in SEQ ID NO: 2; (lllllllllllllll) R, P at a residue corresponding to position 572 in SEQ ID NO: 2; (mmmmmmmmmmmmmmm) T, S at a residue corresponding to position 573 in SEQ ID NO: 2; (nnnnnnnnnnnnnnn) M, L at a residue corresponding to position 574 in SEQ ID NO: 2; (ooooooooooooooo) A, Y, K, T at a residue corresponding to position 575 in SEQ ID NO: 2; (ppppppppppppppp) Y, A, L at a residue corresponding to position 577 in SEQ ID NO: 2; (qqqqqqqqqqqqqqq) Q, A at a residue corresponding to position 578 in SEQ ID NO: 2; (rrrrrrrrrrrrrrr) D at a residue corresponding to position 583 in SEQ ID NO: 2; (sssssssssssssss) N at a residue corresponding to position 584 in SEQ ID NO: 2; (ttttttttttttttt) H, L at a residue corresponding to position 585 in SEQ ID NO: 2; (uuuuuuuuuuuuuuu) A at a residue corresponding to position 587 in SEQ ID NO: 2; (vvvvvvvvvvvvvvv) Q at a residue corresponding to position 588 in SEQ ID NO: 2; (wwwwwwwwwwwwwww) Q, E at a residue corresponding to position 591 in SEQ ID NO: 2; (xxxxxxxxxxxxxxx) A at a residue corresponding to position 593 in SEQ ID NO: 2; (yyyyyyyyyyyyyyy) E at a residue corresponding to position 595 in SEQ ID NO: 2; (zzzzzzzzzzzzzzz) K at a residue corresponding to position 596 in SEQ ID NO: 2; (aaaaaaaaaaaaaaaa) D at a residue corresponding to position 598 in SEQ ID NO: 2; (bbbbbbbbbbbbbbbb) S at a residue corresponding to position 599 in SEQ ID NO: 2; (cccccccccccccccc) L at a residue corresponding to position 601 in SEQ ID NO: 2; or (dddddddddddddddd) any combination thereof.

In some embodiments, an engineered ACL described herein having one or more amino acid alterations results in an engineered ACL comprising: (a) E, R at a residue corresponding to position 5 in SEQ ID NO: 2; (b) L at a residue corresponding to position 6 in SEQ ID NO: 2; (c) K, T at a residue corresponding to position 7 in SEQ ID NO: 2; (d) P at a residue corresponding to position 8 in SEQ ID NO: 2; (e) D, N, R, P, K at a residue corresponding to position 10 in SEQ ID NO: 2; (f) A, V, M, I at a residue corresponding to position 12 in SEQ ID NO: 2; (g) C at a residue corresponding to position 13 in SEQ ID NO: 2; (h) Q, P, A at a residue corresponding to position 14 in SEQ ID NO: 2; (i) S, E at a residue corresponding to position 15 in SEQ ID NO: 2; (j) I, C at a residue corresponding to position 16 in SEQ ID NO: 2; (k) D, R at a residue corresponding to position 19 in SEQ ID NO: 2; (1) H, K, R, D at a residue corresponding to position 20 in SEQ ID NO: 2; (m) E, D at a residue corresponding to position 22 in SEQ ID NO: 2; (n) N at a residue corresponding to position 25 in SEQ ID NO: 2; (o) M at a residue corresponding to position 28 in SEQ ID NO: 2; (p) C at a residue corresponding to position 37 in SEQ ID NO: 2; (q) R at a residue corresponding to position 39 in SEQ ID NO: 2; (r) T at a residue corresponding to position 40 in SEQ ID NO: 2; (s) V, I, M at a residue corresponding to position 41 in SEQ ID NO: 2; (t) S at a residue corresponding to position 43 in SEQ ID NO: 2; (u) I, V at a residue corresponding to position 45 in SEQ ID NO: 2; (v) R, L, I, V, F at a residue corresponding to position 50 in SEQ ID NO: 2; (w) R at a residue corresponding to position 51 in SEQ ID NO: 2; (x) L at a residue corresponding to position 53 in SEQ ID NO: 2; (y) F at a residue corresponding to position 57 in SEQ ID NO: 2; (z) F, M, L, V at a residue corresponding to position 58 in SEQ ID NO: 2; (aa) V at a residue corresponding to position 59 in SEQ ID NO: 2; (bb) P at a residue corresponding to position 61 in SEQ ID NO: 2; (cc) D at a residue corresponding to position 62 in SEQ ID NO: 2; (dd) D at a residue corresponding to position 63 in SEQ ID NO: 2; (ee) K at a residue corresponding to position 67 in SEQ ID NO: 2; (ff) F, L, Y at a residue corresponding to position 69 in SEQ ID NO: 2; (gg) W, F at a residue corresponding to position 71 in SEQ ID NO: 2; (hh) A, R at a residue corresponding to position 72 in SEQ ID NO: 2; (ii) F at a residue corresponding to position 74 in SEQ ID NO: 2; (jj) D at a residue corresponding to position 75 in SEQ ID NO: 2; (kk) D, T, A, R at a residue corresponding to position 76 in SEQ ID NO: 2; (ll) R at a residue corresponding to position 77 in SEQ ID NO: 2; (mm) A at a residue corresponding to position 78 in SEQ ID NO: 2; (nn) K at a residue corresponding to position 80 in SEQ ID NO: 2; (oo) C at a residue corresponding to position 81 in SEQ ID NO: 2; (pp) S, N at a residue corresponding to position 83 in SEQ ID NO: 2; (qq) Y, H at a residue corresponding to position 84 in SEQ ID NO: 2; (rr) F at a residue corresponding to position 85 in SEQ ID NO: 2; (ss) Q, A at a residue corresponding to position 86 in SEQ ID NO: 2; (tt) R at a residue corresponding to position 87 in SEQ ID NO: 2; (uu) L at a residue corresponding to position 88 in SEQ ID NO: 2; (vv) K at a residue corresponding to position 92 in SEQ ID NO: 2; (ww) G at a residue corresponding to position 93 in SEQ ID NO: 2; (xx) R at a residue corresponding to position 94 in SEQ ID NO: 2; (yy) R at a residue corresponding to position 95 in SEQ ID NO: 2; (zz) A at a residue corresponding to position 97 in SEQ ID NO: 2; (aaa) M at a residue corresponding to position 98 in SEQ ID NO: 2; (bbb) L at a residue corresponding to position 99 in SEQ ID NO: 2; (ccc) C at a residue corresponding to position 100 in SEQ ID NO: 2; (ddd) P at a residue corresponding to position 101 in SEQ ID NO: 2; (eee) R at a residue corresponding to position 103 in SEQ ID NO: 2; (fff) A, L, W at a residue corresponding to position 104 in SEQ ID NO: 2; (ggg) F at a residue corresponding to position 106 in SEQ ID NO: 2; (hhh) V at a residue corresponding to position 108 in SEQ ID NO: 2; (iii) V at a residue corresponding to position 109 in SEQ ID NO: 2; (jjj) A, M at a residue corresponding to position 110 in SEQ ID NO: 2; (kkk) C at a residue corresponding to position 113 in SEQ ID NO: 2; (lll) I at a residue corresponding to position 116 in SEQ ID NO: 2; (mmm) T, V, I at a residue corresponding to position 119 in SEQ ID NO: 2; (nnn) Y, V, F, L, M, A at a residue corresponding to position 120 in SEQ ID NO: 2; (ooo) T at a residue corresponding to position 121 in SEQ ID NO: 2; (ppp) I, M, V at a residue corresponding to position 123 in SEQ ID NO: 2; (qqq) S, N, Y at a residue corresponding to position 124 in SEQ ID NO: 2; (rrr) P, T, W at a residue corresponding to position 125 in SEQ ID NO: 2; (sss) R, A, N, M at a residue corresponding to position 126 in SEQ ID NO: 2; (ttt) L, W, F at a residue corresponding to position 127 in SEQ ID NO: 2; (uuu) R at a residue corresponding to position 128 in SEQ ID NO: 2; (vvv) M at a residue corresponding to position 130 in SEQ ID NO: 2; (www) E at a residue corresponding to position 131 in SEQ ID NO: 2; (xxx) E, K, Q at a residue corresponding to position 136 in SEQ ID NO: 2; (yyy) M at a residue corresponding to position 137 in SEQ ID NO: 2; (zzz) M, I, C at a residue corresponding to position 140 in SEQ ID NO: 2; (aaaa) M at a residue corresponding to position 141 in SEQ ID NO: 2; (bbbb) Q, N at a residue corresponding to position 142 in SEQ ID NO: 2; (cccc) R, D, G at a residue corresponding to position 145 in SEQ ID NO: 2; (dddd) S at a residue corresponding to position 147 in SEQ ID NO: 2; (eeee) V, L, A at a residue corresponding to position 148 in SEQ ID NO: 2; (ffff) V at a residue corresponding to position 149 in SEQ ID NO: 2; (gggg) V at a residue corresponding to position 150 in SEQ ID NO: 2; (hhhh) C at a residue corresponding to position 151 in SEQ ID NO: 2; (iiii) E, R at a residue corresponding to position 159 in SEQ ID NO: 2; (jjjj) I at a residue corresponding to position 161 in SEQ ID NO: 2; (kkkk) V at a residue corresponding to position 163 in SEQ ID NO: 2; (llll) A at a residue corresponding to position 164 in SEQ ID NO: 2; (mmmm) R at a residue corresponding to position 165 in SEQ ID NO: 2; (nnnn) R at a residue corresponding to position 167 in SEQ ID NO: 2; (oooo) H at a residue corresponding to position 168 in SEQ ID NO: 2; (pppp) I at a residue corresponding to position 169 in SEQ ID NO: 2; (qqqq) I, L, V at a residue corresponding to position 170 in SEQ ID NO: 2; (rrrr) V at a residue corresponding to position 173 in SEQ ID NO: 2; (ssss) R at a residue corresponding to position 175 in SEQ ID NO: 2; (tttt) D at a residue corresponding to position 176 in SEQ ID NO: 2; (uuuu) E at a residue corresponding to position 177 in SEQ ID NO: 2; (vvvv) E at a residue corresponding to position 181 in SEQ ID NO: 2; (wwww) I at a residue corresponding to position 183 in SEQ ID NO: 2; (xxxx) S at a residue corresponding to position 185 in SEQ ID NO: 2; (yyyy) Y at a residue corresponding to position 186 in SEQ ID NO: 2; (zzzz) D at a residue corresponding to position 187 in SEQ ID NO: 2; (aaaaa) V at a residue corresponding to position 189 in SEQ ID NO: 2; (bbbbb) V at a residue corresponding to position 190 in SEQ ID NO: 2; (ccccc) E, A at a residue corresponding to position 191 in SEQ ID NO: 2; (ddddd) T at a residue corresponding to position 192 in SEQ ID NO: 2; (eeeee) A at a residue corresponding to position 193 in SEQ ID NO: 2; (fffff) S at a residue corresponding to position 194 in SEQ ID NO: 2; (ggggg) S at a residue corresponding to position 195 in SEQ ID NO: 2; (hhhhh) P at a residue corresponding to position 196 in SEQ ID NO: 2; (iiiii) L, A at a residue corresponding to position 201 in SEQ ID NO: 2; (jjjjj) T at a residue corresponding to position 205 in SEQ ID NO: 2; (kkkkk) D at a residue corresponding to position 208 in SEQ ID NO: 2; (lllll) I, T, V at a residue corresponding to position 209 in SEQ ID NO: 2; (mmmmm) L at a residue corresponding to position 211 in SEQ ID NO: 2; (nnnnn) L, M at a residue corresponding to position 212 in SEQ ID NO: 2; (ooooo) I, M, V at a residue corresponding to position 213 in SEQ ID NO: 2; (ppppp) Y at a residue corresponding to position 214 in SEQ ID NO: 2; (qqqqq) S at a residue corresponding to position 215 in SEQ ID NO: 2; (rrrrr) T at a residue corresponding to position 218 in SEQ ID NO: 2; (sssss) T at a residue corresponding to position 219 in SEQ ID NO: 2; (ttttt) G at a residue corresponding to position 220 in SEQ ID NO: 2; (uuuuu) K, L, T, H, R, M at a residue corresponding to position 221 in SEQ ID NO: 2; (vvvvv) S at a residue corresponding to position 222 in SEQ ID NO: 2; (wwwww) A at a residue corresponding to position 224 in SEQ ID NO: 2; (xxxxx) M, V, I at a residue corresponding to position 226 in SEQ ID NO: 2; (yyyyy) I at a residue corresponding to position 227 in SEQ ID NO: 2; (zzzzz) S at a residue corresponding to position 228 in SEQ ID NO: 2; (aaaaaa) H, K at a residue corresponding to position 230 in SEQ ID NO: 2; (bbbbbb) L at a residue corresponding to position 232 in SEQ ID NO: 2; (cccccc) A at a residue corresponding to position 233 in SEQ ID NO: 2; (dddddd) S at a residue corresponding to position 234 in SEQ ID NO: 2; (eeeeee) Q at a residue corresponding to position 235 in SEQ ID NO: 2; (ffffff) M at a residue corresponding to position 236 in SEQ ID NO: 2; (gggggg) A, M, Q at a residue corresponding to position 237 in SEQ ID NO: 2; (hhhhhh) M, H at a residue corresponding to position 238 in SEQ ID NO: 2; (iiiiii) V, L, M at a residue corresponding to position 239 in SEQ ID NO: 2; (jjjjjj) A at a residue corresponding to position 240 in SEQ ID NO: 2; (kkkkkk) Q, E, H, A at a residue corresponding to position 241 in SEQ ID NO: 2; (llllll) C, V, T at a residue corresponding to position 242 in SEQ ID NO: 2; (mmmmmm) W, F, M, I, V at a residue corresponding to position 243 in SEQ ID NO: 2; (nnnnnn) P, Q, G, H, E at a residue corresponding to position 244 in SEQ ID NO: 2; (oooooo) L, M, W at a residue corresponding to position 245 in SEQ ID NO: 2; (pppppp) I at a residue corresponding to position 246 in SEQ ID NO: 2; (qqqqqq) R, P at a residue corresponding to position 247 in SEQ ID NO: 2; (rrrrrr) V, H at a residue corresponding to position 252 in SEQ ID NO: 2; (ssssss) L, V, M at a residue corresponding to position 253 in SEQ ID NO: 2; (tttttt) M, I, V at a residue corresponding to position 254 in SEQ ID NO: 2; (uuuuuu) C, V, T, N at a residue corresponding to position 255 in SEQ ID NO: 2; (vvvvvv) Y, V, F at a residue corresponding to position 256 in SEQ ID NO: 2; (wwwwww) S, H, F, Y at a residue corresponding to position 260 in SEQ ID NO: 2; (xxxxxx) T, C, A at a residue corresponding to position 262 in SEQ ID NO: 2; (yyyyyy) Y at a residue corresponding to position 263 in SEQ ID NO: 2; (zzzzzz) A at a residue corresponding to position 264 in SEQ ID NO: 2; (aaaaaaa) M at a residue corresponding to position 265 in SEQ ID NO: 2; (bbbbbbb) N, M at a residue corresponding to position 266 in SEQ ID NO: 2; (ccccccc) M, F, L, W, G at a residue corresponding to position 267 in SEQ ID NO: 2; (ddddddd) M, A at a residue corresponding to position 268 in SEQ ID NO: 2; (eeeeeee) M at a residue corresponding to position 270 in SEQ ID NO: 2; (fffffff) L, C, A at a residue corresponding to position 271 in SEQ ID NO: 2; (ggggggg) A, V, T, P, C at a residue corresponding to position 272 in SEQ ID NO: 2; (hhhhhhh) I, V, M, F at a residue corresponding to position 273 in SEQ ID NO: 2; (iiiiiii) C, M, H, R, Y at a residue corresponding to position 274 in SEQ ID NO: 2; (jjjjjjj) H, C at a residue corresponding to position 275 in SEQ ID NO: 2; (kkkkkkk) A at a residue corresponding to position 277 in SEQ ID NO: 2; (lllllll) T, A, C at a residue corresponding to position 278 in SEQ ID NO: 2; (mmmmmmm) M, I, V at a residue corresponding to position 279 in SEQ ID NO: 2; (nnnnnnn) V, L at a residue corresponding to position 281 in SEQ ID NO: 2; (ooooooo) E, D, P, R at a residue corresponding to position 283 in SEQ ID NO: 2; (ppppppp) R, F, D at a residue corresponding to position 285 in SEQ ID NO: 2; (qqqqqqq) D, F at a residue corresponding to position 286 in SEQ ID NO: 2; (rrrrrrr) A, P, V at a residue corresponding to position 287 in SEQ ID NO: 2; (sssssss) A at a residue corresponding to position 290 in SEQ ID NO: 2; (ttttttt) M, I, L, V at a residue corresponding to position 291 in SEQ ID NO: 2; (uuuuuuu) H at a residue corresponding to position 292 in SEQ ID NO: 2; (vvvvvvv) A at a residue corresponding to position 294 in SEQ ID NO: 2; (wwwwwww) V, I, M at a residue corresponding to position 295 in SEQ ID NO: 2; (xxxxxxx) Q, D at a residue corresponding to position 296 in SEQ ID NO: 2; (yyyyyyy) M, V, L at a residue corresponding to position 298 in SEQ ID NO: 2; (zzzzzzz) A at a residue corresponding to position 299 in SEQ ID NO: 2; (aaaaaaaa) Q at a residue corresponding to position 300 in SEQ ID NO: 2; (bbbbbbbb) Q at a residue corresponding to position 302 in SEQ ID NO: 2; (cccccccc) A, P, C, V at a residue corresponding to position 303 in SEQ ID NO: 2; (dddddddd) S at a residue corresponding to position 304 in SEQ ID NO: 2; (eeeeeeee) V, M, I at a residue corresponding to position 305 in SEQ ID NO: 2; (ffffffff) I, V, Y at a residue corresponding to position 306 in SEQ ID NO: 2; (gggggggg) M, W, P at a residue corresponding to position 307 in SEQ ID NO: 2; (hhhhhhhh) G at a residue corresponding to position 308 in SEQ ID NO: 2; (iiiiiiii) T, A, R at a residue corresponding to position 311 in SEQ ID NO: 2; (jjjjjjjj) F, V, M at a residue corresponding to position 312 in SEQ ID NO: 2; (kkkkkkkk) F, W at a residue corresponding to position 313 in SEQ ID NO: 2; (llllllll) Q, H, E at a residue corresponding to position 314 in SEQ ID NO: 2; (mmmmmmmm) A, K, L, R at a residue corresponding to position 315 in SEQ ID NO: 2; (nnnnnnnn) I, M at a residue corresponding to position 316 in SEQ ID NO: 2; (oooooooo) D, H at a residue corresponding to position 318 in SEQ ID NO: 2; (pppppppp) R, Q at a residue corresponding to position 321 in SEQ ID NO: 2; (qqqqqqqq) A, K, H, Q at a residue corresponding to position 322 in SEQ ID NO: 2; (rrrrrrrr) F at a residue corresponding to position 330 in SEQ ID NO: 2; (ssssssss) G at a residue corresponding to position 331 in SEQ ID NO: 2; (tttttttt) S at a residue corresponding to position 333 in SEQ ID NO: 2; (uuuuuuuu) V, I, M at a residue corresponding to position 334 in SEQ ID NO: 2; (vvvvvvvv) K at a residue corresponding to position 335 in SEQ ID NO: 2; (wwwwwwww) V, T, M, Y, W, I, F at a residue corresponding to position 336 in SEQ ID NO: 2; (xxxxxxxx) L, V, M at a residue corresponding to position 337 in SEQ ID NO: 2; (yyyyyyyy) L, M, V, W at a residue corresponding to position 338 in SEQ ID NO: 2; (zzzzzzzz) T, S, F at a residue corresponding to position 339 in SEQ ID NO: 2; (aaaaaaaaa) G at a residue corresponding to position 340 in SEQ ID NO: 2; (bbbbbbbbb) S at a residue corresponding to position 342 in SEQ ID NO: 2; (ccccccccc) P, A at a residue corresponding to position 343 in SEQ ID NO: 2; (ddddddddd) I, C, M, V at a residue corresponding to position 344 in SEQ ID NO: 2; (eeeeeeeee) S at a residue corresponding to position 345 in SEQ ID NO: 2; (fffffffff) E, V, A, P at a residue corresponding to position 346 in SEQ ID NO: 2; (ggggggggg) E at a residue corresponding to position 347 in SEQ ID NO: 2; (hhhhhhhhh) I, L, T at a residue corresponding to position 348 in SEQ ID NO: 2; (iiiiiiiii) Y, F, L at a residue corresponding to position 349 in SEQ ID NO: 2; (jjjjjjjjj) R, Q, W at a residue corresponding to position 351 in SEQ ID NO: 2; (kkkkkkkkk) I, M at a residue corresponding to position 352 in SEQ ID NO: 2; (lllllllll) R at a residue corresponding to position 353 in SEQ ID NO: 2; (mmmmmmmmm) K at a residue corresponding to position 354 in SEQ ID NO: 2; (nnnnnnnnn) A at a residue corresponding to position 355 in SEQ ID NO: 2; (ooooooooo) F, I, V, A, G, L, W at a residue corresponding to position 357 in SEQ ID NO: 2; (ppppppppp) Q at a residue corresponding to position 358 in SEQ ID NO: 2; (qqqqqqqqq) Q at a residue corresponding to position 359 in SEQ ID NO: 2; (rrrrrrrrr) C, H, L at a residue corresponding to position 362 in SEQ ID NO: 2; (sssssssss) V, I, G at a residue corresponding to position 363 in SEQ ID NO: 2; (ttttttttt) M, I, V at a residue corresponding to position 365 in SEQ ID NO: 2; (uuuuuuuuu) I, L, F, V at a residue corresponding to position 366 in SEQ ID NO: 2; (vvvvvvvvv) V, I, L, M, T, C at a residue corresponding to position 367 in SEQ ID NO: 2; (wwwwwwwww) N at a residue corresponding to position 368 in SEQ ID NO: 2; (xxxxxxxxx) A, G at a residue corresponding to position 369 in SEQ ID NO: 2; (yyyyyyyyy) Y at a residue corresponding to position 370 in SEQ ID NO: 2; (zzzzzzzzz) C, A at a residue corresponding to position 375 in SEQ ID NO: 2; (aaaaaaaaaa) G, S at a residue corresponding to position 376 in SEQ ID NO: 2; (bbbbbbbbbb) G at a residue corresponding to position 377 in SEQ ID NO: 2; (cccccccccc) T at a residue corresponding to position 378 in SEQ ID NO: 2; (dddddddddd) V, C, L, A, I at a residue corresponding to position 379 in SEQ ID NO: 2; (eeeeeeeeee) S, V, H, T at a residue corresponding to position 380 in SEQ ID NO: 2; (ffffffffff) L, C at a residue corresponding to position 381 in SEQ ID NO: 2; (gggggggggg) T, C, N, L, P at a residue corresponding to position 382 in SEQ ID NO: 2; (hhhhhhhhhh) Y, R at a residue corresponding to position 383 in SEQ ID NO: 2; (iiiiiiiiii) N, W, D at a residue corresponding to position 384 in SEQ ID NO: 2; (jjjjjjjjjj) G at a residue corresponding to position 385 in SEQ ID NO: 2; (kkkkkkkkkk) V, T, L at a residue corresponding to position 386 in SEQ ID NO: 2; (llllllllll) R, H, N, D, E at a residue corresponding to position 387 in SEQ ID NO: 2; (mmmmmmmmmm) K, H at a residue corresponding to position 388 in SEQ ID NO: 2; (nnnnnnnnnn) A, P, L at a residue corresponding to position 389 in SEQ ID NO: 2; (oooooooooo) S, T at a residue corresponding to position 391 in SEQ ID NO: 2; (pppppppppp) V at a residue corresponding to position 392 in SEQ ID NO: 2; (qqqqqqqqqq) I, K at a residue corresponding to position 394 in SEQ ID NO: 2; (rrrrrrrrrr) V at a residue corresponding to position 396 in SEQ ID NO: 2; (ssssssssss) H at a residue corresponding to position 398 in SEQ ID NO: 2; (tttttttttt) C, I, T, V at a residue corresponding to position 399 in SEQ ID NO: 2; (uuuuuuuuuu) Q, D at a residue corresponding to position 400 in SEQ ID NO: 2; (vvvvvvvvvv) L, I, C, A, M at a residue corresponding to position 401 in SEQ ID NO: 2; (wwwwwwwwww) V at a residue corresponding to position 404 in SEQ ID NO: 2; (xxxxxxxxxx) E at a residue corresponding to position 406 in SEQ ID NO: 2; (yyyyyyyyyy) P at a residue corresponding to position 407 in SEQ ID NO: 2; (zzzzzzzzzz) G, Q, E at a residue corresponding to position 409 in SEQ ID NO: 2; (aaaaaaaaaaa) S at a residue corresponding to position 411 in SEQ ID NO: 2; (bbbbbbbbbbb) L, C at a residue corresponding to position 414 in SEQ ID NO: 2; (ccccccccccc) R at a residue corresponding to position 416 in SEQ ID NO: 2; (ddddddddddd) Q, H, M, S at a residue corresponding to position 419 in SEQ ID NO: 2; (eeeeeeeeeee) V at a residue corresponding to position 420 in SEQ ID NO: 2; (fffffffffff) F, T at a residue corresponding to position 421 in SEQ ID NO: 2; (ggggggggggg) L, H at a residue corresponding to position 425 in SEQ ID NO: 2; (hhhhhhhhhhh) A at a residue corresponding to position 430 in SEQ ID NO: 2; (iiiiiiiiiii) K at a residue corresponding to position 432 in SEQ ID NO: 2; (jjjjjjjjjjj) R, D at a residue corresponding to position 433 in SEQ ID NO: 2; (kkkkkkkkkkk) V, T at a residue corresponding to position 434 in SEQ ID NO: 2; (lllllllllll) M at a residue corresponding to position 435 in SEQ ID NO: 2; (mmmmmmmmmmm) F, M at a residue corresponding to position 441 in SEQ ID NO: 2; (nnnnnnnnnnn) Y, R, K, C, H at a residue corresponding to position 442 in SEQ ID NO: 2; (ooooooooooo) S at a residue corresponding to position 443 in SEQ ID NO: 2; (ppppppppppp) L, V, M, I at a residue corresponding to position 446 in SEQ ID NO: 2; (qqqqqqqqqqq) A at a residue corresponding to position 447 in SEQ ID NO: 2; (rrrrrrrrrrr) M, D at a residue corresponding to position 450 in SEQ ID NO: 2; (sssssssssss) E at a residue corresponding to position 451 in SEQ ID NO: 2; (ttttttttttt) P at a residue corresponding to position 452 in SEQ ID NO: 2; (uuuuuuuuuuu) G at a residue corresponding to position 453 in SEQ ID NO: 2; (vvvvvvvvvvv) N, D at a residue corresponding to position 456 in SEQ ID NO: 2; (wwwwwwwwwww) F, I, R, W, Y at a residue corresponding to position 460 in SEQ ID NO: 2; (xxxxxxxxxxx) Y, L at a residue corresponding to position 461 in SEQ ID NO: 2; (yyyyyyyyyyy) V, I at a residue corresponding to position 462 in SEQ ID NO: 2; (zzzzzzzzzzz) D at a residue corresponding to position 463 in SEQ ID NO: 2; (aaaaaaaaaaaa) L at a residue corresponding to position 465 in SEQ ID NO: 2; (bbbbbbbbbbbb) A, D, K at a residue corresponding to position 466 in SEQ ID NO: 2; (cccccccccccc) K at a residue corresponding to position 467 in SEQ ID NO: 2; (dddddddddddd) M at a residue corresponding to position 468 in SEQ ID NO: 2; (eeeeeeeeeeee) L at a residue corresponding to position 469 in SEQ ID NO: 2; (ffffffffffff) V at a residue corresponding to position 470 in SEQ ID NO: 2; (gggggggggggg) T, V at a residue corresponding to position 471 in SEQ ID NO: 2; (hhhhhhhhhhhh) N, S at a residue corresponding to position 473 in SEQ ID NO: 2; (iiiiiiiiiiii) Y, F, V, L, I, N at a residue corresponding to position 476 in SEQ ID NO: 2; (jjjjjjjjjjjj) T at a residue corresponding to position 478 in SEQ ID NO: 2; (kkkkkkkkkkkk) V, P at a residue corresponding to position 479 in SEQ ID NO: 2; (llllllllllll) S, A, P, L at a residue corresponding to position 480 in SEQ ID NO: 2; (mmmmmmmmmmmm) P, E at a residue corresponding to position 481 in SEQ ID NO: 2; (nnnnnnnnnnnn) L, E, R, A at a residue corresponding to position 485 in SEQ ID NO: 2; (oooooooooooo) C at a residue corresponding to position 490 in SEQ ID NO: 2; (pppppppppppp) P at a residue corresponding to position 492 in SEQ ID NO: 2; (qqqqqqqqqqqq) C at a residue corresponding to position 498 in SEQ ID NO: 2; (rrrrrrrrrrrr) V at a residue corresponding to position 507 in SEQ ID NO: 2; (ssssssssssss) V at a residue corresponding to position 508 in SEQ ID NO: 2; (tttttttttttt) A at a residue corresponding to position 510 in SEQ ID NO: 2; (uuuuuuuuuuuu) V, L at a residue corresponding to position 511 in SEQ ID NO: 2; (vvvvvvvvvvvv) I at a residue corresponding to position 512 in SEQ ID NO: 2; (wwwwwwwwwwww) N at a residue corresponding to position 514 in SEQ ID NO: 2; (xxxxxxxxxxxx) D, S at a residue corresponding to position 515 in SEQ ID NO: 2; (yyyyyyyyyyyy) A at a residue corresponding to position 516 in SEQ ID NO: 2; (zzzzzzzzzzzz) I at a residue corresponding to position 518 in SEQ ID NO: 2; (aaaaaaaaaaaaa) T at a residue corresponding to position 519 in SEQ ID NO: 2; (bbbbbbbbbbbbb) P, W at a residue corresponding to position 521 in SEQ ID NO: 2; (ccccccccccccc) M, L at a residue corresponding to position 526 in SEQ ID NO: 2; (ddddddddddddd) L at a residue corresponding to position 527 in SEQ ID NO: 2; (eeeeeeeeeeeee) R, H at a residue corresponding to position 532 in SEQ ID NO: 2; (fffffffffffff) L at a residue corresponding to position 535 in SEQ ID NO: 2; (ggggggggggggg) Q at a residue corresponding to position 540 in SEQ ID NO: 2; (hhhhhhhhhhhhh) Y, W at a residue corresponding to position 542 in SEQ ID NO: 2; (iiiiiiiiiiiii) N at a residue corresponding to position 546 in SEQ ID NO: 2; (jjjjjjjjjjjjj) Q at a residue corresponding to position 552 in SEQ ID NO: 2; (kkkkkkkkkkkkk) E at a residue corresponding to position 554 in SEQ ID NO: 2; (lllllllllllll) D at a residue corresponding to position 560 in SEQ ID NO: 2; (mmmmmmmmmmmmm) D at a residue corresponding to position 561 in SEQ ID NO: 2; (nnnnnnnnnnnnn) L, F at a residue corresponding to position 563 in SEQ ID NO: 2; (ooooooooooooo) S, T, L, D, R, K at a residue corresponding to position 564 in SEQ ID NO: 2; (ppppppppppppp) T, M, P, V, I at a residue corresponding to position 565 in SEQ ID NO: 2; (qqqqqqqqqqqqq) D, A, E, P at a residue corresponding to position 566 in SEQ ID NO: 2; (rrrrrrrrrrrrr) A, S, Q, N, T, K at a residue corresponding to position 567 in SEQ ID NO: 2; (sssssssssssss) G, D, N at a residue corresponding to position 568 in SEQ ID NO: 2; (ttttttttttttt) M, K at a residue corresponding to position 569 in SEQ ID NO: 2; (uuuuuuuuuuuuu) V, L at a residue corresponding to position 570 in SEQ ID NO: 2; (vvvvvvvvvvvvv) P at a residue corresponding to position 572 in SEQ ID NO: 2; (wwwwwwwwwwwww) S, T at a residue corresponding to position 573 in SEQ ID NO: 2; (xxxxxxxxxxxxx) M, L at a residue corresponding to position 574 in SEQ ID NO: 2; (yyyyyyyyyyyyy) T, Y at a residue corresponding to position 575 in SEQ ID NO: 2; (zzzzzzzzzzzzz) A, L at a residue corresponding to position 577 in SEQ ID NO: 2; (aaaaaaaaaaaaaa) A, Q at a residue corresponding to position 578 in SEQ ID NO: 2; (bbbbbbbbbbbbbb) D at a residue corresponding to position 583 in SEQ ID NO: 2; (cccccccccccccc) N at a residue corresponding to position 584 in SEQ ID NO: 2; (dddddddddddddd) H at a residue corresponding to position 585 in SEQ ID NO: 2; (eeeeeeeeeeeeee) A at a residue corresponding to position 587 in SEQ ID NO: 2; (ffffffffffffff) Q at a residue corresponding to position 588 in SEQ ID NO: 2; (gggggggggggggg) Q, E at a residue corresponding to position 591 in SEQ ID NO: 2; (hhhhhhhhhhhhhh) A at a residue corresponding to position 593 in SEQ ID NO: 2; (iiiiiiiiiiiiii) E at a residue corresponding to position 595 in SEQ ID NO: 2; (jjjjjjjjjjjjjj) K at a residue corresponding to position 596 in SEQ ID NO: 2; (kkkkkkkkkkkkkk) D at a residue corresponding to position 598 in SEQ ID NO: 2; (llllllllllllll) S at a residue corresponding to position 599 in SEQ ID NO: 2; (mmmmmmmmmmmmmm) L at a residue corresponding to position 601 in SEQ ID NO: 2; or (nnnnnnnnnnnnnn) Any combination thereof.

In some embodiments, an engineered ACL described herein having one or more amino acid alterations results in an engineered ACL comprising: (a) E, R at a residue corresponding to position 5 in SEQ ID NO: 2; (b) L at a residue corresponding to position 6 in SEQ ID NO: 2; (c) K, T at a residue corresponding to position 7 in SEQ ID NO: 2; (d) P at a residue corresponding to position 8 in SEQ ID NO: 2; (e) D, N, R, P at a residue corresponding to position 10 in SEQ ID NO: 2; (f) A, V, M at a residue corresponding to position 12 in SEQ ID NO: 2; (g) Q, P at a residue corresponding to position 14 in SEQ ID NO: 2; (h) S, E at a residue corresponding to position 15 in SEQ ID NO: 2; (i) I at a residue corresponding to position 16 in SEQ ID NO: 2; (j) D, R at a residue corresponding to position 19 in SEQ ID NO: 2; (k) H, K, R, D at a residue corresponding to position 20 in SEQ ID NO: 2; (1) E, D at a residue corresponding to position 22 in SEQ ID NO: 2; (m) M at a residue corresponding to position 28 in SEQ ID NO: 2; (n) R at a residue corresponding to position 39 in SEQ ID NO: 2; (o) V at a residue corresponding to position 41 in SEQ ID NO: 2; (p) S at a residue corresponding to position 43 in SEQ ID NO: 2; (q) I at a residue corresponding to position 45 in SEQ ID NO: 2; (r) R, L, I, V, F at a residue corresponding to position 50 in SEQ ID NO: 2; (s) F at a residue corresponding to position 57 in SEQ ID NO: 2; (t) F, M, L, V at a residue corresponding to position 58 in SEQ ID NO: 2; (u) P at a residue corresponding to position 61 in SEQ ID NO: 2; (v) D at a residue corresponding to position 62 in SEQ ID NO: 2; (w) D at a residue corresponding to position 63 in SEQ ID NO: 2; (x) K at a residue corresponding to position 67 in SEQ ID NO: 2; (y) F, L, Y at a residue corresponding to position 69 in SEQ ID NO: 2; (z) W, F at a residue corresponding to position 71 in SEQ ID NO: 2; (aa) A, R at a residue corresponding to position 72 in SEQ ID NO: 2; (bb) D, T, A, R at a residue corresponding to position 76 in SEQ ID NO: 2; (cc) R at a residue corresponding to position 77 in SEQ ID NO: 2; (dd) A at a residue corresponding to position 78 in SEQ ID NO: 2; (ee) C at a residue corresponding to position 81 in SEQ ID NO: 2; (ff) Y at a residue corresponding to position 84 in SEQ ID NO: 2; (gg) F at a residue corresponding to position 85 in SEQ ID NO: 2; (hh) Q at a residue corresponding to position 86 in SEQ ID NO: 2; (ii) R at a residue corresponding to position 87 in SEQ ID NO: 2; (jj) K at a residue corresponding to position 92 in SEQ ID NO: 2; (kk) G at a residue corresponding to position 93 in SEQ ID NO: 2; (ll) R at a residue corresponding to position 94 in SEQ ID NO: 2; (mm) R at a residue corresponding to position 95 in SEQ ID NO: 2; (nn) M at a residue corresponding to position 98 in SEQ ID NO: 2; (oo) A at a residue corresponding to position 104 in SEQ ID NO: 2; (pp) V at a residue corresponding to position 108 in SEQ ID NO: 2; (qq) V at a residue corresponding to position 109 in SEQ ID NO: 2; (rr) C at a residue corresponding to position 113 in SEQ ID NO: 2; (ss) I at a residue corresponding to position 116 in SEQ ID NO: 2; (tt) T at a residue corresponding to position 119 in SEQ ID NO: 2; (uu) Y, V, F, L, M, A at a residue corresponding to position 120 in SEQ ID NO: 2; (vv) I at a residue corresponding to position 123 in SEQ ID NO: 2; (ww) S, N at a residue corresponding to position 124 in SEQ ID NO: 2; (xx) P, T, W at a residue corresponding to position 125 in SEQ ID NO: 2; (yy) R, A, N at a residue corresponding to position 126 in SEQ ID NO: 2; (zz) L, W at a residue corresponding to position 127 in SEQ ID NO: 2; (aaa) M at a residue corresponding to position 130 in SEQ ID NO: 2; (bbb) E at a residue corresponding to position 131 in SEQ ID NO: 2; (ccc) E, K at a residue corresponding to position 136 in SEQ ID NO: 2; (ddd) M, I, C at a residue corresponding to position 140 in SEQ ID NO: 2; (eee) M at a residue corresponding to position 141 in SEQ ID NO: 2; (fff) Q, N at a residue corresponding to position 142 in SEQ ID NO: 2; (ggg) R at a residue corresponding to position 145 in SEQ ID NO: 2; (hhh) S at a residue corresponding to position 147 in SEQ ID NO: 2; (iii) V at a residue corresponding to position 148 in SEQ ID NO: 2; (jjj) V at a residue corresponding to position 149 in SEQ ID NO: 2; (kkk) C at a residue corresponding to position 151 in SEQ ID NO: 2; (lll) E, R at a residue corresponding to position 159 in SEQ ID NO: 2; (mmm) I at a residue corresponding to position 161 in SEQ ID NO: 2; (nnn) V at a residue corresponding to position 163 in SEQ ID NO: 2; (ooo) A at a residue corresponding to position 164 in SEQ ID NO: 2; (ppp) R at a residue corresponding to position 167 in SEQ ID NO: 2; (qqq) H at a residue corresponding to position 168 in SEQ ID NO: 2; (rrr) I at a residue corresponding to position 169 in SEQ ID NO: 2; (sss) I, L, V at a residue corresponding to position 170 in SEQ ID NO: 2; (ttt) V at a residue corresponding to position 173 in SEQ ID NO: 2; (uuu) R at a residue corresponding to position 175 in SEQ ID NO: 2; (vvv) D at a residue corresponding to position 176 in SEQ ID NO: 2; (www) E at a residue corresponding to position 177 in SEQ ID NO: 2; (xxx) E at a residue corresponding to position 181 in SEQ ID NO: 2; (yyy) I at a residue corresponding to position 183 in SEQ ID NO: 2; (zzz) S at a residue corresponding to position 185 in SEQ ID NO: 2; (aaaa) Y at a residue corresponding to position 186 in SEQ ID NO: 2; (bbbb) D at a residue corresponding to position 187 in SEQ ID NO: 2; (cccc) V at a residue corresponding to position 190 in SEQ ID NO: 2; (dddd) E, A at a residue corresponding to position 191 in SEQ ID NO: 2; (eeee) A at a residue corresponding to position 193 in SEQ ID NO: 2; (ffff) S at a residue corresponding to position 194 in SEQ ID NO: 2; (gggg) S at a residue corresponding to position 195 in SEQ ID NO: 2; (hhhh) L, A at a residue corresponding to position 201 in SEQ ID NO: 2; (iiii) T at a residue corresponding to position 205 in SEQ ID NO: 2; (jjjj) I at a residue corresponding to position 209 in SEQ ID NO: 2; (kkkk) Y at a residue corresponding to position 214 in SEQ ID NO: 2; (1111) T at a residue corresponding to position 218 in SEQ ID NO: 2; (mmmm) T at a residue corresponding to position 219 in SEQ ID NO: 2; (nnnn) G at a residue corresponding to position 220 in SEQ ID NO: 2; (oooo) K, L, T, H at a residue corresponding to position 221 in SEQ ID NO: 2; (pppp) M, V at a residue corresponding to position 226 in SEQ ID NO: 2; (qqqq) S at a residue corresponding to position 228 in SEQ ID NO: 2; (rrrr) H, K at a residue corresponding to position 230 in SEQ ID NO: 2; (ssss) L at a residue corresponding to position 232 in SEQ ID NO: 2; (tttt) S at a residue corresponding to position 234 in SEQ ID NO: 2; (uuuu) A at a residue corresponding to position 237 in SEQ ID NO: 2; (vvvv) M at a residue corresponding to position 238 in SEQ ID NO: 2; (wwww) V at a residue corresponding to position 239 in SEQ ID NO: 2; (xxxx) Q, E at a residue corresponding to position 241 in SEQ ID NO: 2; (yyyy) C, V, T at a residue corresponding to position 242 in SEQ ID NO: 2; (zzzz) W, F, M at a residue corresponding to position 243 in SEQ ID NO: 2; (aaaaa) P, Q at a residue corresponding to position 244 in SEQ ID NO: 2; (bbbbb) I at a residue corresponding to position 246 in SEQ ID NO: 2; (ccccc) R at a residue corresponding to position 247 in SEQ ID NO: 2; (ddddd) L, V at a residue corresponding to position 253 in SEQ ID NO: 2; (eeeee) M, I, V at a residue corresponding to position 254 in SEQ ID NO: 2; (fffff) S, H at a residue corresponding to position 260 in SEQ ID NO: 2; (ggggg) T at a residue corresponding to position 262 in SEQ ID NO: 2; (hhhhh) Y at a residue corresponding to position 263 in SEQ ID NO: 2; (iiiii) N at a residue corresponding to position 266 in SEQ ID NO: 2; (jjjjj) M, F at a residue corresponding to position 267 in SEQ ID NO: 2; (kkkkk) A at a residue corresponding to position 272 in SEQ ID NO: 2; (lllll) I, V, M, F at a residue corresponding to position 273 in SEQ ID NO: 2; (mmmmm) C at a residue corresponding to position 274 in SEQ ID NO: 2; (nnnnn) T, A at a residue corresponding to position 278 in SEQ ID NO: 2; (ooooo) M at a residue corresponding to position 279 in SEQ ID NO: 2; (ppppp) V at a residue corresponding to position 281 in SEQ ID NO: 2; (qqqqq) E at a residue corresponding to position 283 in SEQ ID NO: 2; (rrrrr) R at a residue corresponding to position 285 in SEQ ID NO: 2; (sssss) A at a residue corresponding to position 290 in SEQ ID NO: 2; (ttttt) M, I, L, V at a residue corresponding to position 291 in SEQ ID NO: 2; (uuuuu) H at a residue corresponding to position 292 in SEQ ID NO: 2; (vvvvv) V, I, M at a residue corresponding to position 295 in SEQ ID NO: 2; (wwwww) Q at a residue corresponding to position 296 in SEQ ID NO: 2; (xxxxx) M, V at a residue corresponding to position 298 in SEQ ID NO: 2; (yyyyy) Q at a residue corresponding to position 300 in SEQ ID NO: 2; (zzzzz) Q at a residue corresponding to position 302 in SEQ ID NO: 2; (aaaaaa) A, P at a residue corresponding to position 303 in SEQ ID NO: 2; (bbbbbb) S at a residue corresponding to position 304 in SEQ ID NO: 2; (cccccc) V, M, I at a residue corresponding to position 305 in SEQ ID NO: 2; (dddddd) M at a residue corresponding to position 307 in SEQ ID NO: 2; (eeeeee) T, A at a residue corresponding to position 311 in SEQ ID NO: 2; (ffffff) F at a residue corresponding to position 313 in SEQ ID NO: 2; (gggggg) Q, H at a residue corresponding to position 314 in SEQ ID NO: 2; (hhhhhh) A, K, L, R at a residue corresponding to position 315 in SEQ ID NO: 2; (iiiiii) I at a residue corresponding to position 316 in SEQ ID NO: 2; (jjjjjj) D, H at a residue corresponding to position 318 in SEQ ID NO: 2; (kkkkkk) R, Q at a residue corresponding to position 321 in SEQ ID NO: 2; (llllll) A, K, H, Q at a residue corresponding to position 322 in SEQ ID NO: 2; (mmmmmm) F at a residue corresponding to position 330 in SEQ ID NO: 2; (nnnnnn) S at a residue corresponding to position 333 in SEQ ID NO: 2; (oooooo) V, I at a residue corresponding to position 334 in SEQ ID NO: 2; (pppppp) V, T, M, Y at a residue corresponding to position 336 in SEQ ID NO: 2; (qqqqqq) L, V at a residue corresponding to position 337 in SEQ ID NO: 2; (rrrrrr) L, M, V at a residue corresponding to position 338 in SEQ ID NO: 2; (ssssss) T at a residue corresponding to position 339 in SEQ ID NO: 2; (tttttt) G at a residue corresponding to position 340 in SEQ ID NO: 2; (uuuuuu) I, C, M at a residue corresponding to position 344 in SEQ ID NO: 2; (vvvvvv) S at a residue corresponding to position 345 in SEQ ID NO: 2; (wwwwww) E at a residue corresponding to position 346 in SEQ ID NO: 2; (xxxxxx) E at a residue corresponding to position 347 in SEQ ID NO: 2; (yyyyyy) I, L, T at a residue corresponding to position 348 in SEQ ID NO: 2; (zzzzzz) Y, F at a residue corresponding to position 349 in SEQ ID NO: 2; (aaaaaaa) R, Q at a residue corresponding to position 351 in SEQ ID NO: 2; (bbbbbbb) I at a residue corresponding to position 352 in SEQ ID NO: 2; (ccccccc) R at a residue corresponding to position 353 in SEQ ID NO: 2; (ddddddd) K at a residue corresponding to position 354 in SEQ ID NO: 2; (eeeeeee) A at a residue corresponding to position 355 in SEQ ID NO: 2; (fffffff) F, I, V, A, G, L, W at a residue corresponding to position 357 in SEQ ID NO: 2; (ggggggg) Q at a residue corresponding to position 358 in SEQ ID NO: 2; (hhhhhhh) Q at a residue corresponding to position 359 in SEQ ID NO: 2; (iiiiiii) C, H at a residue corresponding to position 362 in SEQ ID NO: 2; (jjjjjjj) V, I at a residue corresponding to position 363 in SEQ ID NO: 2; (kkkkkkk) M at a residue corresponding to position 365 in SEQ ID NO: 2; (lllllll) V, I, L, M at a residue corresponding to position 367 in SEQ ID NO: 2; (mmmmmmm) A at a residue corresponding to position 369 in SEQ ID NO: 2; (nnnnnnn) C at a residue corresponding to position 375 in SEQ ID NO: 2; (ooooooo) G, S at a residue corresponding to position 376 in SEQ ID NO: 2; (ppppppp) V, C, L at a residue corresponding to position 379 in SEQ ID NO: 2; (qqqqqqq) T, C at a residue corresponding to position 382 in SEQ ID NO: 2; (rrrrrrr) Y at a residue corresponding to position 383 in SEQ ID NO: 2; (sssssss) N at a residue corresponding to position 384 in SEQ ID NO: 2; (ttttttt) V, T at a residue corresponding to position 386 in SEQ ID NO: 2; (uuuuuuu) R, H, N, D at a residue corresponding to position 387 in SEQ ID NO: 2; (vvvvvvv) K, H at a residue corresponding to position 388 in SEQ ID NO: 2; (wwwwwww) A, P, L at a residue corresponding to position 389 in SEQ ID NO: 2; (xxxxxxx) C, I, T, V at a residue corresponding to position 399 in SEQ ID NO: 2; (yyyyyyy) Q at a residue corresponding to position 400 in SEQ ID NO: 2; (zzzzzzz) L, I, C at a residue corresponding to position 401 in SEQ ID NO: 2; (aaaaaaaa) V at a residue corresponding to position 404 in SEQ ID NO: 2; (bbbbbbbb) P at a residue corresponding to position 407 in SEQ ID NO: 2; (cccccccc) G at a residue corresponding to position 409 in SEQ ID NO: 2; (dddddddd) R at a residue corresponding to position 416 in SEQ ID NO: 2; (eeeeeeee) Q at a residue corresponding to position 419 in SEQ ID NO: 2; (ffffffff) V at a residue corresponding to position 420 in SEQ ID NO: 2; (gggggggg) F at a residue corresponding to position 421 in SEQ ID NO: 2; (hhhhhhhh) A at a residue corresponding to position 430 in SEQ ID NO: 2; (iiiiiiii) K at a residue corresponding to position 432 in SEQ ID NO: 2; (jjjjjjjj) R, D at a residue corresponding to position 433 in SEQ ID NO: 2; (kkkkkkkk) V at a residue corresponding to position 434 in SEQ ID NO: 2; (llllllll) F at a residue corresponding to position 441 in SEQ ID NO: 2; (mmmmmmmm) Y, R, K, C, H at a residue corresponding to position 442 in SEQ ID NO: 2; (nnnnnnnn) L, V, M, I at a residue corresponding to position 446 in SEQ ID NO: 2; (oooooooo) M at a residue corresponding to position 450 in SEQ ID NO: 2; (pppppppp) P at a residue corresponding to position 452 in SEQ ID NO: 2; (qqqqqqqq) G at a residue corresponding to position 453 in SEQ ID NO: 2; (rrrrrrrr) N, D at a residue corresponding to position 456 in SEQ ID NO: 2; (ssssssss) F, I, R at a residue corresponding to position 460 in SEQ ID NO: 2; (tttttttt) Y at a residue corresponding to position 461 in SEQ ID NO: 2; (uuuuuuuu) L at a residue corresponding to position 465 in SEQ ID NO: 2; (vvvvvvvv) A, D at a residue corresponding to position 466 in SEQ ID NO: 2; (wwwwwwww) T at a residue corresponding to position 471 in SEQ ID NO: 2; (xxxxxxxx) N, S at a residue corresponding to position 473 in SEQ ID NO: 2; (yyyyyyyy) Y, F, V, L, I at a residue corresponding to position 476 in SEQ ID NO: 2; (zzzzzzzz) V, P at a residue corresponding to position 479 in SEQ ID NO: 2; (aaaaaaaaa) S, A, P at a residue corresponding to position 480 in SEQ ID NO: 2; (bbbbbbbbb) P, E at a residue corresponding to position 481 in SEQ ID NO: 2; (ccccccccc) L, E, R, A at a residue corresponding to position 485 in SEQ ID NO: 2; (ddddddddd) C at a residue corresponding to position 490 in SEQ ID NO: 2; (eeeeeeeee) P at a residue corresponding to position 492 in SEQ ID NO: 2; (fffffffff) V at a residue corresponding to position 507 in SEQ ID NO: 2; (ggggggggg) V at a residue corresponding to position 511 in SEQ ID NO: 2; (hhhhhhhhh) N at a residue corresponding to position 514 in SEQ ID NO: 2; (iiiiiiiii) D, S at a residue corresponding to position 515 in SEQ ID NO: 2; (jjjjjjjjj) A at a residue corresponding to position 516 in SEQ ID NO: 2; (kkkkkkkkk) I at a residue corresponding to position 518 in SEQ ID NO: 2; (lllllllll) T at a residue corresponding to position 519 in SEQ ID NO: 2; (mmmmmmmmm) P, W at a residue corresponding to position 521 in SEQ ID NO: 2; (nnnnnnnnn) M, L at a residue corresponding to position 526 in SEQ ID NO: 2; (ooooooooo) L at a residue corresponding to position 527 in SEQ ID NO: 2; (ppppppppp) R, H at a residue corresponding to position 532 in SEQ ID NO: 2; (qqqqqqqqq) Q at a residue corresponding to position 540 in SEQ ID NO: 2; (rrrrrrrrr) Y at a residue corresponding to position 542 in SEQ ID NO: 2; (sssssssss) N at a residue corresponding to position 546 in SEQ ID NO: 2; (ttttttttt) E at a residue corresponding to position 554 in SEQ ID NO: 2; (uuuuuuuuu) D at a residue corresponding to position 561 in SEQ ID NO: 2; (vvvvvvvvv) S, T at a residue corresponding to position 564 in SEQ ID NO: 2; (wwwwwwwww) T, M, P, V, I at a residue corresponding to position 565 in SEQ ID NO: 2; (xxxxxxxxx) D at a residue corresponding to position 566 in SEQ ID NO: 2; (yyyyyyyyy) A, S, Q, N at a residue corresponding to position 567 in SEQ ID NO: 2; (zzzzzzzzz) G, D, N at a residue corresponding to position 568 in SEQ ID NO: 2; (aaaaaaaaaa) V at a residue corresponding to position 570 in SEQ ID NO: 2; (bbbbbbbbbb) P at a residue corresponding to position 572 in SEQ ID NO: 2; (cccccccccc) M at a residue corresponding to position 574 in SEQ ID NO: 2; (dddddddddd) T at a residue corresponding to position 575 in SEQ ID NO: 2; (eeeeeeeeee) A at a residue corresponding to position 577 in SEQ ID NO: 2; (ffffffffff) N at a residue corresponding to position 584 in SEQ ID NO: 2; (gggggggggg) A at a residue corresponding to position 587 in SEQ ID NO: 2; (hhhhhhhhhh) E at a residue corresponding to position 595 in SEQ ID NO: 2; (iiiiiiiiii) K at a residue corresponding to position 596 in SEQ ID NO: 2; (jjjjjjjjjj) S at a residue corresponding to position 599 in SEQ ID NO: 2; or (kkkkkkkkkk) Any combination thereof.

In some embodiments, an engineered ACL described herein having one or more amino acid alterations results in an engineered ACL comprising: (a) E, R at a residue corresponding to position 5 in SEQ ID NO: 2; (b) L at a residue corresponding to position 6 in SEQ ID NO: 2; (c) K, T at a residue corresponding to position 7 in SEQ ID NO: 2; (d) P at a residue corresponding to position 8 in SEQ ID NO: 2; (e) D, N at a residue corresponding to position 10 in SEQ ID NO: 2; (f) A at a residue corresponding to position 12 in SEQ ID NO: 2; (g) Q, P at a residue corresponding to position 14 in SEQ ID NO: 2; (h) S, E at a residue corresponding to position 15 in SEQ ID NO: 2; (i) D at a residue corresponding to position 19 in SEQ ID NO: 2; (j) H, K, R at a residue corresponding to position 20 in SEQ ID NO: 2; (k) E at a residue corresponding to position 22 in SEQ ID NO: 2; (1) V at a residue corresponding to position 41 in SEQ ID NO: 2; (m) I at a residue corresponding to position 45 in SEQ ID NO: 2; (n) R, L, I, V at a residue corresponding to position 50 in SEQ ID NO: 2; (o) F, M, L at a residue corresponding to position 58 in SEQ ID NO: 2; (p) D at a residue corresponding to position 62 in SEQ ID NO: 2; (q) D at a residue corresponding to position 63 in SEQ ID NO: 2; (r) K at a residue corresponding to position 67 in SEQ ID NO: 2; (s) F, L, Y at a residue corresponding to position 69 in SEQ ID NO: 2; (t) A, R at a residue corresponding to position 72 in SEQ ID NO: 2; (u) D, T, A, R at a residue corresponding to position 76 in SEQ ID NO: 2; (v) R at a residue corresponding to position 77 in SEQ ID NO: 2; (w) C at a residue corresponding to position 81 in SEQ ID NO: 2; (x) Q at a residue corresponding to position 86 in SEQ ID NO: 2; (y) R at a residue corresponding to position 87 in SEQ ID NO: 2; (z) K at a residue corresponding to position 92 in SEQ ID NO: 2; (aa) G at a residue corresponding to position 93 in SEQ ID NO: 2; (bb) R at a residue corresponding to position 94 in SEQ ID NO: 2; (cc) R at a residue corresponding to position 95 in SEQ ID NO: 2; (dd) M at a residue corresponding to position 98 in SEQ ID NO: 2; (ee) A at a residue corresponding to position 104 in SEQ ID NO: 2; (ff) V at a residue corresponding to position 108 in SEQ ID NO: 2; (gg) C at a residue corresponding to position 113 in SEQ ID NO: 2; (hh) I at a residue corresponding to position 116 in SEQ ID NO: 2; (ii) Y, V, F, L at a residue corresponding to position 120 in SEQ ID NO: 2; (jj) I at a residue corresponding to position 123 in SEQ ID NO: 2; (kk) S at a residue corresponding to position 124 in SEQ ID NO: 2; (ll) P, T at a residue corresponding to position 125 in SEQ ID NO: 2; (mm) R, A, N at a residue corresponding to position 126 in SEQ ID NO: 2; (nn) L at a residue corresponding to position 127 in SEQ ID NO: 2; (oo) M at a residue corresponding to position 130 in SEQ ID NO: 2; (pp) E at a residue corresponding to position 136 in SEQ ID NO: 2; (qq) M, I at a residue corresponding to position 140 in SEQ ID NO: 2; (rr) M at a residue corresponding to position 141 in SEQ ID NO: 2; (ss) Q, N at a residue corresponding to position 142 in SEQ ID NO: 2; (tt) R at a residue corresponding to position 145 in SEQ ID NO: 2; (uu) S at a residue corresponding to position 147 in SEQ ID NO: 2; (vv) V at a residue corresponding to position 148 in SEQ ID NO: 2; (ww) V at a residue corresponding to position 149 in SEQ ID NO: 2; (xx) C at a residue corresponding to position 151 in SEQ ID NO: 2; (yy) E at a residue corresponding to position 159 in SEQ ID NO: 2; (zz) I at a residue corresponding to position 161 in SEQ ID NO: 2; (aaa) V at a residue corresponding to position 163 in SEQ ID NO: 2; (bbb) A at a residue corresponding to position 164 in SEQ ID NO: 2; (ccc) R at a residue corresponding to position 167 in SEQ ID NO: 2; (ddd) H at a residue corresponding to position 168 in SEQ ID NO: 2; (eee) I at a residue corresponding to position 169 in SEQ ID NO: 2; (fff) R at a residue corresponding to position 175 in SEQ ID NO: 2; (ggg) D at a residue corresponding to position 176 in SEQ ID NO: 2; (hhh) E at a residue corresponding to position 177 in SEQ ID NO: 2; (iii) S at a residue corresponding to position 185 in SEQ ID NO: 2; (jjj) Y at a residue corresponding to position 186 in SEQ ID NO: 2; (kkk) D at a residue corresponding to position 187 in SEQ ID NO: 2; (lll) V at a residue corresponding to position 190 in SEQ ID NO: 2; (mmm) E, A at a residue corresponding to position 191 in SEQ ID NO: 2; (nnn) A at a residue corresponding to position 193 in SEQ ID NO: 2; (ooo) S at a residue corresponding to position 195 in SEQ ID NO: 2; (ppp) L, A at a residue corresponding to position 201 in SEQ ID NO: 2; (qqq) T at a residue corresponding to position 205 in SEQ ID NO: 2; (rrr) I at a residue corresponding to position 209 in SEQ ID NO: 2; (sss) Y at a residue corresponding to position 214 in SEQ ID NO: 2; (ttt) T at a residue corresponding to position 219 in SEQ ID NO: 2; (uuu) G at a residue corresponding to position 220 in SEQ ID NO: 2; (vvv) K at a residue corresponding to position 221 in SEQ ID NO: 2; (www) S at a residue corresponding to position 228 in SEQ ID NO: 2; (xxx) H, K at a residue corresponding to position 230 in SEQ ID NO: 2; (yyy) L at a residue corresponding to position 232 in SEQ ID NO: 2; (zzz) S at a residue corresponding to position 234 in SEQ ID NO: 2; (aaaa) A at a residue corresponding to position 237 in SEQ ID NO: 2; (bbbb) M at a residue corresponding to position 238 in SEQ ID NO: 2; (cccc) V at a residue corresponding to position 239 in SEQ ID NO: 2; (dddd) Q, E at a residue corresponding to position 241 in SEQ ID NO: 2; (eeee) C, V, T at a residue corresponding to position 242 in SEQ ID NO: 2; (ffff) W, F, M at a residue corresponding to position 243 in SEQ ID NO: 2; (gggg) P, Q at a residue corresponding to position 244 in SEQ ID NO: 2; (hhhh) I at a residue corresponding to position 246 in SEQ ID NO: 2; (iiii) R at a residue corresponding to position 247 in SEQ ID NO: 2; (jjjj) L at a residue corresponding to position 253 in SEQ ID NO: 2; (kkkk) M at a residue corresponding to position 254 in SEQ ID NO: 2; (llll) S, H at a residue corresponding to position 260 in SEQ ID NO: 2; (mmmm) Y at a residue corresponding to position 263 in SEQ ID NO: 2; (nnnn) N at a residue corresponding to position 266 in SEQ ID NO: 2; (oooo) M, F at a residue corresponding to position 267 in SEQ ID NO: 2; (pppp) A at a residue corresponding to position 272 in SEQ ID NO: 2; (qqqq) I at a residue corresponding to position 273 in SEQ ID NO: 2; (rrrr) C at a residue corresponding to position 274 in SEQ ID NO: 2; (ssss) T, A at a residue corresponding to position 278 in SEQ ID NO: 2; (tttt) M at a residue corresponding to position 279 in SEQ ID NO: 2; (uuuu) V at a residue corresponding to position 281 in SEQ ID NO: 2; (vvvv) E at a residue corresponding to position 283 in SEQ ID NO: 2; (wwww) M, I, L, V at a residue corresponding to position 291 in SEQ ID NO: 2; (xxxx) H at a residue corresponding to position 292 in SEQ ID NO: 2; (yyyy) V, I, M at a residue corresponding to position 295 in SEQ ID NO: 2; (zzzz) M, V at a residue corresponding to position 298 in SEQ ID NO: 2; (aaaaa) Q at a residue corresponding to position 300 in SEQ ID NO: 2; (bbbbb) Q at a residue corresponding to position 302 in SEQ ID NO: 2; (ccccc) A, P at a residue corresponding to position 303 in SEQ ID NO: 2; (ddddd) S at a residue corresponding to position 304 in SEQ ID NO: 2; (eeeee) V at a residue corresponding to position 305 in SEQ ID NO: 2; (fffff) M at a residue corresponding to position 307 in SEQ ID NO: 2; (ggggg) T, A at a residue corresponding to position 311 in SEQ ID NO: 2; (hhhhh) F at a residue corresponding to position 313 in SEQ ID NO: 2; (iiiii) Q, H at a residue corresponding to position 314 in SEQ ID NO: 2; (jjjjj) A, K at a residue corresponding to position 315 in SEQ ID NO: 2; (kkkkk) D at a residue corresponding to position 318 in SEQ ID NO: 2; (lllll) R, Q at a residue corresponding to position 321 in SEQ ID NO: 2; (mmmmm) A, K, H, Q at a residue corresponding to position 322 in SEQ ID NO: 2; (nnnnn) F at a residue corresponding to position 330 in SEQ ID NO: 2; (ooooo) S at a residue corresponding to position 333 in SEQ ID NO: 2; (ppppp) V, I at a residue corresponding to position 334 in SEQ ID NO: 2; (qqqqq) V, T, M at a residue corresponding to position 336 in SEQ ID NO: 2; (rrrrr) L, V at a residue corresponding to position 337 in SEQ ID NO: 2; (sssss) L, M at a residue corresponding to position 338 in SEQ ID NO: 2; (ttttt) G at a residue corresponding to position 340 in SEQ ID NO: 2; (uuuuu) I at a residue corresponding to position 344 in SEQ ID NO: 2; (vvvvv) I, L at a residue corresponding to position 348 in SEQ ID NO: 2; (wwwww) Y at a residue corresponding to position 349 in SEQ ID NO: 2; (xxxxx) R, Q at a residue corresponding to position 351 in SEQ ID NO: 2; (yyyyy) R at a residue corresponding to position 353 in SEQ ID NO: 2; (zzzzz) K at a residue corresponding to position 354 in SEQ ID NO: 2; (aaaaaa) A at a residue corresponding to position 355 in SEQ ID NO: 2; (bbbbbb) F, I at a residue corresponding to position 357 in SEQ ID NO: 2; (cccccc) Q at a residue corresponding to position 358 in SEQ ID NO: 2; (dddddd) Q at a residue corresponding to position 359 in SEQ ID NO: 2; (eeeeee) C, H at a residue corresponding to position 362 in SEQ ID NO: 2; (ffffff) V at a residue corresponding to position 363 in SEQ ID NO: 2; (gggggg) M at a residue corresponding to position 365 in SEQ ID NO: 2; (hhhhhh) V, I, L, M at a residue corresponding to position 367 in SEQ ID NO: 2; (iiiiii) A at a residue corresponding to position 369 in SEQ ID NO: 2; (jjjjjj) G at a residue corresponding to position 376 in SEQ ID NO: 2; (kkkkkk) V, C at a residue corresponding to position 379 in SEQ ID NO: 2; (llllll) T, C at a residue corresponding to position 382 in SEQ ID NO: 2; (mmmmmm) Y at a residue corresponding to position 383 in SEQ ID NO: 2; (nnnnnn) V, T at a residue corresponding to position 386 in SEQ ID NO: 2; (oooooo) R, H, N at a residue corresponding to position 387 in SEQ ID NO: 2; (pppppp) K at a residue corresponding to position 388 in SEQ ID NO: 2; (qqqqqq) A, P at a residue corresponding to position 389 in SEQ ID NO: 2; (rrrrrr) C, I, T, V at a residue corresponding to position 399 in SEQ ID NO: 2; (ssssss) Q at a residue corresponding to position 400 in SEQ ID NO: 2; (tttttt) L, I at a residue corresponding to position 401 in SEQ ID NO: 2; (uuuuuu) V at a residue corresponding to position 404 in SEQ ID NO: 2; (vvvvvv) P at a residue corresponding to position 407 in SEQ ID NO: 2; (wwwwww) G at a residue corresponding to position 409 in SEQ ID NO: 2; (xxxxxx) V at a residue corresponding to position 420 in SEQ ID NO: 2; (yyyyyy) F at a residue corresponding to position 421 in SEQ ID NO: 2; (zzzzzz) A at a residue corresponding to position 430 in SEQ ID NO: 2; (aaaaaaa) K at a residue corresponding to position 432 in SEQ ID NO: 2; (bbbbbbb) R, D at a residue corresponding to position 433 in SEQ ID NO: 2; (ccccccc) V at a residue corresponding to position 434 in SEQ ID NO: 2; (ddddddd) Y, R, K, C, H at a residue corresponding to position 442 in SEQ ID NO: 2; (eeeeeee) L, V, M, I at a residue corresponding to position 446 in SEQ ID NO: 2; (fffffff) M at a residue corresponding to position 450 in SEQ ID NO: 2; (ggggggg) P at a residue corresponding to position 452 in SEQ ID NO: 2; (hhhhhhh) G at a residue corresponding to position 453 in SEQ ID NO: 2; (iiiiiii) N, D at a residue corresponding to position 456 in SEQ ID NO: 2; (jjjjjjj) F, I at a residue corresponding to position 460 in SEQ ID NO: 2; (kkkkkkk) Y at a residue corresponding to position 461 in SEQ ID NO: 2; (lllllll) L at a residue corresponding to position 465 in SEQ ID NO: 2; (mmmmmmm) A at a residue corresponding to position 466 in SEQ ID NO: 2; (nnnnnnn) N, S at a residue corresponding to position 473 in SEQ ID NO: 2; (ooooooo) Y, F, V, L, I at a residue corresponding to position 476 in SEQ ID NO: 2; (ppppppp) V, P at a residue corresponding to position 479 in SEQ ID NO: 2; (qqqqqqq) S, A at a residue corresponding to position 480 in SEQ ID NO: 2; (rrrrrrr) P, E at a residue corresponding to position 481 in SEQ ID NO: 2; (sssssss) L, E, R, A at a residue corresponding to position 485 in SEQ ID NO: 2; (ttttttt) P at a residue corresponding to position 492 in SEQ ID NO: 2; (uuuuuuu) V at a residue corresponding to position 507 in SEQ ID NO: 2; (vvvvvvv) N at a residue corresponding to position 514 in SEQ ID NO: 2; (wwwwwww) D, S at a residue corresponding to position 515 in SEQ ID NO: 2; (xxxxxxx) A at a residue corresponding to position 516 in SEQ ID NO: 2; (yyyyyyy) I at a residue corresponding to position 518 in SEQ ID NO: 2; (zzzzzzz) T at a residue corresponding to position 519 in SEQ ID NO: 2; (aaaaaaaa) P at a residue corresponding to position 521 in SEQ ID NO: 2; (bbbbbbbb) M, L at a residue corresponding to position 526 in SEQ ID NO: 2; (cccccccc) L at a residue corresponding to position 527 in SEQ ID NO: 2; (dddddddd) R, H at a residue corresponding to position 532 in SEQ ID NO: 2; (eeeeeeee) Q at a residue corresponding to position 540 in SEQ ID NO: 2; (ffffffff) Y at a residue corresponding to position 542 in SEQ ID NO: 2; (gggggggg) E at a residue corresponding to position 554 in SEQ ID NO: 2; (hhhhhhhh) S, T at a residue corresponding to position 564 in SEQ ID NO: 2; (iiiiiiii) T, M, P, V, I at a residue corresponding to position 565 in SEQ ID NO: 2; (jjjjjjjj) A, S, Q, N at a residue corresponding to position 567 in SEQ ID NO: 2; (kkkkkkkk) G, D at a residue corresponding to position 568 in SEQ ID NO: 2; (llllllll) V at a residue corresponding to position 570 in SEQ ID NO: 2; (mmmmmmmm) P at a residue corresponding to position 572 in SEQ ID NO: 2; (nnnnnnnn) T at a residue corresponding to position 575 in SEQ ID NO: 2; (oooooooo) N at a residue corresponding to position 584 in SEQ ID NO: 2; (pppppppp) A at a residue corresponding to position 587 in SEQ ID NO: 2; (qqqqqqqq) E at a residue corresponding to position 595 in SEQ ID NO: 2; (rrrrrrrr) K at a residue corresponding to position 596 in SEQ ID NO: 2; or (ssssssss) any combination thereof.

In some embodiments, an engineered ACL described herein includes one or more amino acid alterations selected from the group consisting of: D5R, D5E, A6L, S7K, S7T, G8P, E10K, E10D, E10P, E10N, E10R, L12A, L12I, L12V, L12M, P13C, H14P, H14A, H14Q, D15E, D15L, D15S, D15R, V16C, V16L, V16I, E19D, E19R, S20H, S20K, S20R, S20D, P22D, P22E, S25N, L28M, V37C, P39R, N40T, T41M, T41L, T41V, T41I, A43S, W44M, L45I, L45V, W48P, K50I, K50F, K50R, K50V, K50L, E51R, P53L, A57F, I58V, I58L, I58M, I58F, A59V, R61P, R61E, A62D, G63D, W66G, R67K, H69F, H69Y, H69L, Y71F, Y71W, N72A, N72R, L74F, L75D, N76T, N76D, N76A, N76R, M77R, V78A, N80K, V81C, G83S, G83H, G83Q, G83N, A84Y, A84H, L85F, L86A, L86Q, A87R, R88L, A92K, D93G, S94R, P95R, V97A, V98M, M99L, S100C, G101P, G103R, V104L, V104W, V104A, H106W, H106F, A107V, A107P, A107I, A107L, A107W, A107F, I108V, L109V, S110M, S110F, S110Y, S110A, S110D, A113T, A113C, A113V, Q114M, Q114W, Q114L, Q114G, Q114A, Y115A, V116I, P119V, P119T, P119I, I120M, I120F, I120V, I120A, I120L, I120W, I120Y, V121T, P122A, L123M, L123V, L123I, A124Y, A124S, A124N, E125P, E125T, E125W, Q126M, Q126N, Q126R, Q126A, Y127F, Y127W, Y127L, S128R, I130M, P131E, R136E, R136Q, R136K, L137M, V140M, V140C, V140I, L141M, E142N, E142Q, K145G, K145R, K145D, A147S, M148L, M148V, M148A, A149V, A149L, F150V, V151C, Y157L, S159R, S159E, L161I, L163V, P164A, Q165R, E167R, G168H, V169I, E170I, E170V, E170L, A173V, K175R, T176D, D177E, V181E, V183I, A185S, F186Y, A187D, L189V, L190V, G191A, G191E, H192T, G193A, Q194S, G195S, G196P, H201A, H201P, H201L, G205T, T208D, L209I, L209T, L209P, L209V, K211L, K211T, K211C, K211M, K211V, K211A, I212L, I212M, L213I, L213C, L213V, L213Q, L213M, F214Y, T215S, S216G, S218T, S219T, S220G, D221T, D221M, D221L, D221R, D221K, D221H, P222S, G224A, V225A, L226M, L226I, L226V, T227L, T227D, T227H, T227I, T228S, R230H, R230K, M231N, M232L, M232I, M232V, C233L, C233V, C233M, C233W, C233A, C233I, A234S, A234W, N235Q, N235S, Q236A, Q236C, Q236I, Q236M, Q236V, T237A, T237M, T237Q, T237R, T237E, Q238M, Q238S, Q238W, Q238N, Q238H, 1239F, 1239M, 1239V, 1239C, 1239L, S240A, S240R, D241A, D241H, D241E, D241Q, A242V, A242C, A242T, A242W, L243I, L243F, L243M, L243W, L243V, R244E, R244P, R244Q, R244H, R244G, F245M, F245L, F245H, F245W, F245Y, L246I, L246T, L246M, K247R, K247P, K247E, P251E, R252H, R252V, I253Y, I253M, I253F, I253L, I253V, L254I, L254F, L254V, L254M, D255A, D255T, D255N, D255L, D255I, D255G, D255H, D255C, D255V, D255S, W256F, W256V, W256I, W256C, W256Y, L257M, W259L, W259F, N260H, N260S, N260Y, N260F, V262C, V262T, V262A, V262I, V262M, F263A, F263Y, F263H, G264A, G265M, G265L, G265W, G265F, G265C, S266N, S266T, S266M, S266V, H267I, H267G, H267M, H267W, H267F, H267L, H267V, H267C, H267A, N268M, N268A, N268C, N268G, N268V, F269L, F269M, F269I, F269V, N270M, N270H, N270Y, N270T, M271A, M271P, M271C, M271T, M271L, M272C, M272V, M272P, M272A, M272T, L273M, L273V, L273F, L273I, A274Y, A274R, A274V, A274M, A274C, A274H, A274W, N275G, N275C, N275S, N275H, G277A, G277C, S278C, S278T, S278A, S278H, L279V, L279M, L279I, I281F, I281L, I281V, I281M, D282P, D282M, D282E, D282A, D282H, N283E, N283D, N283P, N283R, G284F, G284R, G284W, K285R, K285F, K285W, K285D, P286D, P286F, T287P, T287A, T287V, G290A, F291M, F291V, F291L, F291P, F291I, E292H, T294I, T294L, T294A, L295W, L295M, L295V, L295I, R296D, R296Q, I298M, I298V, I298L, K299A, E300Q, P302Q, G303V, G303I, G303A, G303P, G303C, T304S, L305W, L305Y, L305V, L305I, L305F, L305H, L305M, A306V, A306Y, A306I, A306M, A306L, A306F, F307P, F307W, F307Y, F307H, F307M, N308T, N308A, N308S, N308L, N308G, N308V, N308M, V311A, V311R, V311P, V311T, G312I, G312M, G312V, G312F, Y313L, Y313F, Y313W, A314Q, A314E, A314H, M315R, M315A, M315K, M315L, L316W, L316M, L316I, L316F, N318H, N318D, E321R, E321Q, R322Q, R322K, R322A, R322H, Y330L, Y330F, F331G, E333S, L334I, L334M, L334V, D335R, D335K, L336T, L336I, L336M, L336Y, L336V, L336F, L336W, I337L, I337M, I337F, I337A, I337V, F338W, F338L, F338M, F338V, Y339F, Y339T, Y339S, Y339C, A340G, G341A, G341S, A342S, S343P, S343A, L344M, L344I, L344V, L344C, P345S, Q346V, Q346A, Q346P, Q346E, D347E, V348T, V348I, V348L, W349M, W349F, W349L, W349A, W3491, W349Y, S351F, S351R, S351Q, S351W, L352I, L352M, L352Y, E353R, R354K, F355A, F355R, M357W, M357G, M357V, M357L, M357P, M357I, M357F, M357A, E358Q, E359H, E359Q, G361F, S362H, S362L, S362C, L363D, L363V, L363I, L363G, L365M, L365V, L365I, M366I, M366V, M366L, M366F, A367T, A367C, A367M, A367I, A367V, A367L, S368N, S368Q, S368E, S368T, S369G, S369A, W370Y, W370F, M372L, M372Q, T373S, T375A, T375C, A376T, A376S, A376C, A376G, P377G, P377M, A378C, A378T, A378V, T379C, T379V, T379I, T379L, T379A, I380F, I380C, I380H, I380S, I380M, I380T, I380V, M381N, M381F, M381L, M381C, M381V, V382L, V382N, V382C, V382T, V382P, H383D, H383N, H383R, H383P, H383Y, E384P, E384N, E384D, E384W, P385G, P385D, I386D, I386V, I386L, I386T, G387D, G387R, G387N, G387H, G387E, R388K, R388H, S389A, S389P, S389L, V391S, V391T, I392V, I392C, V394R, V394K, V394I, V394Q, V394L, P395A, L396F, L396I, L396M, L396V, G398D, G398H, G398N, A399M, A399V, A399T, A399C, A399I, E400D, E400Q, V401M, V401I, V401C, V401L, V401A, K402R, L404V, P405D, D406E, D407P, M409Q, M409E, M409G, C411D, C411S, C411V, C411G, C411N, L413I, L413V, R414W, R414L, R414V, R414C, R414Q, V415I, K416R, G417S, P418D, N419H, N419S, N419Q, N419M, I420V, M421T, M421F, F425H, F425L, K430A, A432K, E433R, E433D, A434T, A434V, F435I, F435M, F435L, L441F, L441M, L441Y, V442K, V442H, V442Y, V442R, V442C, T443S, A446M, A446I, A446L, A446V, V447A, V447G, F449I, V450D, V450M, D451E, D452P, A453G, P455E, P455G, P455R, S456D, S456E, S456Q, S456N, G458N, G458F, G458Y, G458W, L459Y, V460Y, V460F, V460I, V460R, V460W, F461I, F461L, F461Y, D462I, D462T, D462V, G463D, R464D, V465L, S466D, S466K, S466A, E467K, D468M, F469L, K470V, L471T, L471V, T473S, T473N, G474E, W476N, W476Y, W476L, W476F, W476V, W476I, Q478T, Q478L, A479P, A479V, G480L, G480I, G480S, G480P, G480A, G480V, V481P, V481E, R483E, D485A, D485L, D485E, D485R, L490C, G492P, L493F, D496H, V497A, V498C, H502P, R504H, I507Y, I507V, I507W, G508V, F510A, 1511V, I511L, F512I, P513V, L514N, P515D, P515S, G516A, V518I, S519T, T521P, T521W, V526L, V526M, I527L, S532R, S532H, I535L, R540Q, M542Y, M542W, A546N, R552Q, N554E, I558F, S560D, E561D, P563F, P563L, P563W, Q564D, Q564K, Q564S, Q564L, Q564T, Q564R, L565M, L565I, L565V, L565P, L565T, K566A, K566E, K566D, K566P, D567K, D567Q, D567T, D567N, D567S, D567A, H568G, H568N, H568D, E569M, E569K, I570V, I570L, D572R, D572P, K573T, K573S, G574M, G574L, S575A, S575Y, S575K, S575T, N577Y, N577A, N577L, I578Q, I578A, T583D, R584N, R585H, R585L, D587A, L588Q, R591Q, R591E, Y593A, N595E, E596K, P598D, A599S, I601L, and any combination thereof.

In some embodiments, an engineered ACL described herein includes one or more amino acid alterations selected from the group consisting of: D5E, D5R, A6L, S7K, S7T, G8P, E10D, E10N, E10R, E10P, E10K, L12A, L12V, L12M, L12I, P13C, H14Q, H14P, H14A, D15S, D15E, V161, V16C, E19D, E19R, S20H, S20K, S20R, S20D, P22E, P22D, S25N, L28M, V37C, P39R, N40T, T41V, T41I, T41M, A43S, L45I, L45V, K50R, K50L, K50I, K50V, K50F, E51R, P53L, A57F, I58F, I58M, I58L, I58V, A59V, R61P, A62D, G63D, R67K, H69F, H69L, H69Y, Y71W, Y71F, N72A, N72R, L74F, L75D, N76D, N76T, N76A, N76R, M77R, V78A, N80K, V81C, G83S, G83N, A84Y, A84H, L85F, L86Q, L86A, A87R, R88L, A92K, D93G, S94R, P95R, V97A, V98M, M99L, S100C, G101P, G103R, V104A, V104L, V104W, H106F, I108V, L109V, S110A, S110M, A113C, V116I, P119T, P119V, P119I, I120Y, I120V, I120F, I120L, I120M, I120A, V121T, L123I, L123M, L123V, A124S, A124N, A124Y, E125P, E125T, E125W, Q126R, Q126A, Q126N, Q126M, Y127L, Y127W, Y127F, S128R, I130M, P131E, R136E, R136K, R136Q, L137M, V140M, V140I, V140C, L141M, E142Q, E142N, K145R, K145D, K145G, A147S, M148V, M148L, M148A, A149V, F150V, V151C, S159E, S159R, L161I, L163V, P164A, Q165R, E167R, G168H, V169I, E170I, E170L, E170V, A173V, K175R, T176D, D177E, V181E, V183I, A185S, F186Y, A187D, L189V, L190V, G191E, G191A, H192T, G193A, Q194S, G195S, G196P, H201L, H201A, G205T, T208D, L209I, L209T, L209V, K211L, I212L, I212M, L213I, L213M, L213V, F214Y, T215S, S218T, S219T, S220G, D221K, D221L, D221T, D221H, D221R, D221M, P222S, G224A, L226M, L226V, L226I, T227I, T228S, R230H, R230K, M232L, C233A, A234S, N235Q, Q236M, T237A, T237M, T237Q, Q238M, Q238H, I239V, I239L, I239M, S240A, D241Q, D241E, D241H, D241A, A242C, A242V, A242T, L243W, L243F, L243M, L243I, L243V, R244P, R244Q, R244G, R244H, R244E, F245L, F245M, F245W, L246I, K247R, K247P, R252V, R252H, I253L, I253V, I253M, L254M, L254I, L254V, D255C, D255V, D255T, D255N, W256Y, W256V, W256F, N260S, N260H, N260F, N260Y, V262T, V262C, V262A, F263Y, G264A, G265M, S266N, S266M, H267M, H267F, H267L, H267W, H267G, N268M, N268A, N270M, M271L, M271C, M271A, M272A, M272V, M272T, M272P, M272C, L273I, L273V, L273M, L273F, A274C, A274M, A274H, A274R, A274Y, N275H, N275C, G277A, S278T, S278A, S278C, L279M, L279I, L279V, I281V, I281L, N283E, N283D, N283P, N283R, K285R, K285F, K285D, P286D, P286F, T287A, T287P, T287V, G290A, F291M, F291I, F291L, F291V, E292H, T294A, L295V, L295I, L295M, R296Q, R296D, I298M, I298V, I298L, K299A, E300Q, P302Q, G303A, G303P, G303C, G303V, T304S, L305V, L305M, L305I, A306I, A306V, A306Y, F307M, F307W, F307P, N308G, V311T, V311A, V311R, G312F, G312V, G312M, Y313F, Y313W, A314Q, A314H, A314E, M315A, M315K, M315L, M315R, L316I, L316M, N318D, N318H, E321R, E321Q, R322A, R322K, R322H, R322Q, Y330F, F331G, E333S, L334V, L334I, L334M, D335K, L336V, L336T, L336M, L336Y, L336W, L336I, L336F, I337L, I337V, I337M, F338L, F338M, F338V, F338W, Y339T, Y339S, Y339F, A340G, A342S, S343P, S343A, L344I, L344C, L344M, L344V, P345S, Q346E, Q346V, Q346A, Q346P, D347E, V348I, V348L, V348T, W349Y, W349F, W349L, S351R, S351Q, S351W, L352I, L352M, E353R, R354K, F355A, M357F, M357I, M357V, M357A, M357G, M357L, M357W, E358Q, E359Q, S362C, S362H, S362L, L363V, L363I, L363G, L365M, L365I, L365V, M366I, M366L, M366F, M366V, A367V, A367I, A367L, A367M, A367T, A367C, S368N, S369A, S369G, W370Y, T375C, T375A, A376G, A376S, P377G, A378T, T379V, T379C, T379L, T379A, T379I, I380S, I380V, I380H, I380T, M381L, M381C, V382T, V382C, V382N, V382L, V382P, H383Y, H383R, E384N, E384W, E384D, P385G, I386V, I386T, I386L, G387R, G387H, G387N, G387D, G387E, R388K, R388H, S389A, S389P, S389L, V391S, V391T, I392V, V394I, V394K, L396V, G398H, A399C, A399I, A399T, A399V, E400Q, E400D, V401L, V401I, V401C, V401A, V401M, L404V, D406E, D407P, M409G, M409Q, M409E, C411S, R414L, R414C, K416R, N419Q, N419H, N419M, N419S, I420V, M421F, M421T, F425L, F425H, K430A, A432K, E433R, E433D, A434V, A434T, F435M, L441F, L441M, V442Y, V442R, V442K, V442C, V442H, T443S, A446L, A446V, A446M, A446I, V447A, V450M, V450D, D451E, D452P, A453G, S456N, S456D, V460F, V460I, V460R, V460W, V460Y, F461Y, F461L, D462V, D462I, G463D, V465L, S466A, S466D, S466K, E467K, D468M, F469L, K470V, L471T, L471V, T473N, T473S, W476Y, W476F, W476V, W476L, W476I, W476N, Q478T, A479V, A479P, G480S, G480A, G480P, G480L, V481P, V481E, D485L, D485E, D485R, D485A, L490C, G492P, V498C, I507V, G508V, F510A, I511V, I511L, F512I, L514N, P515D, P515S, G516A, V518I, S519T, T521P, T521W, V526M, V526L, I527L, S532R, S532H, I535L, R540Q, M542Y, M542W, A546N, R552Q, N554E, S560D, E561D, P563L, P563F, Q564S, Q564T, Q564L, Q564D, Q564R, Q564K, L565T, L565M, L565P, L565V, L565I, K566D, K566A, K566E, K566P, D567A, D567S, D567Q, D567N, D567T, D567K, H568G, H568D, H568N, E569M, E569K, I570V, I570L, D572P, K573S, K573T, G574M, G574L, S575T, S575Y, N577A, N577L, I578A, I578Q, T583D, R584N, R585H, D587A, L588Q, R591Q, R591E, Y593A, N595E, E596K, P598D, A599S, 1601L and any combination thereof.

In some embodiments, an engineered ACL described herein includes one or more amino acid alterations selected from the group consisting of: D5E, D5R, A6L, S7K, S7T, G8P, E10D, E10N, E10R, E10P, L12A, L12V, L12M, H14Q, H14P, D15S, D15E, V16I, E19D, E19R, S20H, S20K, S20R, S20D, P22E, P22D, L28M, P39R, T41V, A43S, L45I, K50R, K50L, K50I, K50V, K50F, A57F, I58F, I58M, I58L, I58V, R61P, A62D, G63D, R67K, H69F, H69L, H69Y, Y71W, Y71F, N72A, N72R, N76D, N76T, N76A, N76R, M77R, V78A, V81C, A84Y, L85F, L86Q, A87R, A92K, D93G, S94R, P95R, V98M, V104A, I108V, L109V, A113C, V116I, P119T, I120Y, I120V, I120F, I120L, I120M, I120A, L123I, A124S, A124N, E125P, E125T, E125W, Q126R, Q126A, Q126N, Y127L, Y127W, I130M, P131E, R136E, R136K, V140M, V140I, V140C, L141M, E142Q, E142N, K145R, A147S, M148V, A149V, V151C, S159E, S159R, L161I, L163V, P164A, E167R, G168H, V169I, E170I, E170L, E170V, A173V, K175R, T176D, D177E, V181E, V183I, A185S, F186Y, A187D, L190V, G191E, G191A, G193A, Q194S, G195S, H201L, H201A, G205T, L209I, F214Y, S218T, S219T, S220G, D221K, D221L, D221T, D221H, L226M, L226V, T228S, R230H, R230K, M232L, A234S, T237A, Q238M, I239V, D241Q, D241E, A242C, A242V, A242T, L243W, L243F, L243M, R244P, R244Q, L246I, K247R, I253L, I253V, L254M, L254I, L254V, N260S, N260H, V262T, F263Y, S266N, H267M, H267F, M272A, L273I, L273V, L273M, L273F, A274C, S278T, S278A, L279M, I281V, N283E, K285R, G290A, F291M, F291I, F291L, F291V, E292H, L295V, L295I, L295M, R296Q, I298M, I298V, E300Q, P302Q, G303A, G303P, T304S, L305V, L305M, L305I, F307M, V311T, V311A, Y313F, A314Q, A314H, M315A, M315K, M315L, M315R, L316I, N318D, N318H, E321R, E321Q, R322A, R322K, R322H, R322Q, Y330F, E333S, L334V, L334I, L336V, L336T, L336M, L336Y, I337L, I337V, F338L, F338M, F338V, Y339T, A340G, L344I, L344C, L344M, P345S, Q346E, D347E, V348I, V348L, V348T, W349Y, W349F, S351R, S351Q, L352I, E353R, R354K, F355A, M357F, M357I, M357V, M357A, M357G, M357L, M357W, E358Q, E359Q, S362C, S362H, L363V, L363I, L365M, A367V, A367I, A367L, A367M, S369A, T375C, A376G, A376S, T379V, T379C, T379L, V382T, V382C, H383Y, E384N, I386V, I386T, G387R, G387H, G387N, G387D, R388K, R388H, S389A, S389P, S389L, A399C, A399I, A399T, A399V, E400Q, V401L, V401I, V401C, L404V, D407P, M409G, K416R, N419Q, I420V, M421F, K430A, A432K, E433R, E433D, A434V, L441F, V442Y, V442R, V442K, V442C, V442H, A446L, A446V, A446M, A446I, V450M, D452P, A453G, S456N, S456D, V460F, V460I, V460R, F461Y, V465L, S466A, S466D, L471T, T473N, T473S, W476Y, W476F, W476V, W476L, W476I, A479V, A479P, G480S, G480A, G480P, V481P, V481E, D485L, D485E, D485R, D485A, L490C, G492P, I507V, I511V, L514N, P515D, P515S, G516A, V518I, S519T, T521P, T521W, V526M, V526L, I527L, S532R, S532H, R540Q, M542Y, A546N, N554E, E561D, Q564S, Q564T, L565T, L565M, L565P, L565V, L565I, K566D, D567A, D567S, D567Q, D567N, H568G, H568D, H568N, I570V, D572P, G574M, S575T, N577A, R584N, D587A, N595E, E596K, A599S, and any combination thereof.

In some embodiments, an engineered ACL describes herein includes one or more amino acid alterations selected from the group consisting of: D5E, D5R, A6L, S7K, S7T, G8P, E10D, E10N, L12A, H14Q, H14P, D15S, D15E, E19D, S20H, S20K, S20R, P22E, T41V, L45I, K50R, K50L, K50I, K50V, I58F, I58M, I58L, A62D, G63D, R67K, H69F, H69L, H69Y, N72A, N72R, N76D, N76T, N76A, N76R, M77R, V81C, L86Q, A87R, A92K, D93G, S94R, P95R, V98M, V104A, I108V, A113C, V116I, I120Y, I120V, I120F, I120L, L123I, A124S, E125P, E125T, Q126R, Q126A, Q126N, Y127L, I130M, R136E, V140M, V140I, L141M, E142Q, E142N, K145R, A147S, M148V, A149V, V151C, S159E, L161I, L163V, P164A, E167R, G168H, V169I, K175R, T176D, D177E, A185S, F186Y, A187D, L190V, G191E, G191A, G193A, G195S, H201L, H201A, G205T, L209I, F214Y, S219T, S220G, D221K, T228S, R230H, R230K, M232L, A234S, T237A, Q238M, I239V, D241Q, D241E, A242C, A242V, A242T, L243W, L243F, L243M, R244P, R244Q, L246I, K247R, I253L, L254M, N260S, N260H, F263Y, S266N, H267M, H267F, M272A, L273I, A274C, S278T, S278A, L279M, I281V, N283E, F291M, F291I, F291L, F291V, E292H, L295V, L295I, L295M, I298M, I298V, E300Q, P302Q, G303A, G303P, T304S, L305V, F307M, V311T, V311A, Y313F, A314Q, A314H, M315A, M315K, N318D, E321R, E321Q, R322A, R322K, R322H, R322Q, Y330F, E333S, L334V, L334I, L336V, L336T, L336M, I337L, I337V, F338L, F338M, A340G, L344I, V348I, V348L, W349Y, S351R, S351Q, E353R, R354K, F355A, M357F, M357I, E358Q, E359Q, S362C, S362H, L363V, L365M, A367V, A367I, A367L, A367M, S369A, A376G, T379V, T379C, V382T, V382C, H383Y, I386V, I386T, G387R, G387H, G387N, R388K, S389A, S389P, A399C, A399I, A399T, A399V, E400Q, V401L, V401I, L404V, D407P, M409G, I420V, M421F, K430A, A432K, E433R, E433D, A434V, V442Y, V442R, V442K, V442C, V442H, A446L, A446V, A446M, A446I, V450M, D452P, A453G, S456N, S456D, V460F, V460I, F461Y, V465L, S466A, T473N, T473S, W476Y, W476F, W476V, W476L, W476I, A479V, A479P, G480S, G480A, V481P, V481E, D485L, D485E, D485R, D485A, G492P, I507V, L514N, P515D, P515S, G516A, V518I, S519T, T521P, V526M, V526L, I527L, S532R, S532H, R540Q, M542Y, N554E, Q564S, Q564T, L565T, L565M, L565P, L565V, L565I, D567A, D567S, D567Q, D567N, H568G, H568D, I570V, D572P, S575T, R584N, D587A, N595E, E596K, and any combination thereof.

In some embodiments, an engineered ACL described herein includes a single alteration or a combination of alterations described in TABLE 9.

In some embodiments, an engineered ACL described herein includes an amino acid sequence, other than the one or more amino acid alterations, has at least 65%, at least 70%, at least 75%, at least 80%, at least 85%, at least 90%, at least 95%, at least 98%, at least 99%, or 100% sequence identity to the amino acid sequence referenced in SEQ ID NO: 1 or SEQ ID NO: 2.

In some embodiments, described herein is a recombinant nucleic acid encoding an engineered ACL described herein. The recombinant nucleic acid can include a nucleotide sequence encoding an engineered ACL operatively linked to a promoter. In some embodiments, described herein is a vector including a recombinant nucleic acid encoding an engineered ACL described herein and/or a nucleotide sequence encoding an engineered ACL operatively linked to a promoter.

In some embodiments, provided herein is an engineered aldehyde dehydrogenase (ALDH) that is a variant of amino acid sequence SEQ ID NO: 3 or a functional fragment thereof, wherein the engineered ALDH comprises one or more alterations at a position described in TABLE 12. An engineered ALDH described herein, in some embodiments, is capable of: (a) catalyzing the conversion of HHA-CoA to 6-hydroxyhexanal (6-HHAI); (b) catalyzing the conversion of NADH to NAD+; (c) catalyzing the conversion of HHA-CoA to 6-HHAI at a greater rate as compared to the wild-type ALDH; (d) having a higher affinity for a HHA-CoA substrate as compared to the wild-type ALDH; (e) catalyzing the conversion of 6ACA-CoA to 6-aminocaproate semialdehyde (6ACA-SADH); (f) catalyzing the conversion of 6ACA-CoA to 6ACA-SADH at a greater rate as compared to the wild-type ALDH; (g) having a higher affinity for a 6ACA-CoA substrate as compared to the wild-type ALDH; or (h) any combination thereof. In some embodiments, an engineered ALDH described herein is capable of: (a) catalyzing the conversion of adipyl-CoA to adipate semialdehyde; (b) catalyzing the conversion of NADH to NAD+; (c) catalyzing the conversion of adipyl-CoA to adipate semialdehyde at a greater rate as compared to the wild-type ALDH; (d) having a higher affinity for an adipyl-CoA substrate as compared to the wild-type ALDH; (e) or any combination thereof. In some embodiments, an engineered ALDH described herein has higher specificity for conversion of HHA-CoA to 6-HHAI, or 6ACA-CoA to 6ACA-SADH, or both, over conversion of: (a) adipyl-CoA to adipate semialdehyde; (b) acetyl-CoA to acetaldehyde; (c) succinyl-CoA to succinate semialdehyde; (d) 3-hydroxyadipyl-CoA to 4-hydroxy-6-oxohexanoate; (e) 3HB-CoA to 3-hydroxybutyrate aldehyde (3HB-Ald); or (f) any combination thereof. An engineered ALDH described herein, in some embodiments, has higher specificity for conversion of HHA-CoA to 6-HHAI over conversion of acetyl-CoA to acetaldehyde, succinyl-CoA to succinate semialdehyde, or both. An engineered ALDH described herein, in some embodiments, has higher specificity for conversion of adipyl-CoA to adipate semialdehyde over conversion of acetyl-CoA to acetaldehyde, succinyl-CoA to succinate semialdehyde, or both. An engineered ALDH described herein, in some embodiments, has a lower or equal affinity for succinyl-CoA as compared to the wild-type ALDH.

In some embodiments, an engineered ALDH described herein includes an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an ALDH consisting of the amino acid sequence of SEQ ID NO: 3.

In some embodiments, an engineered ALDH described herein having one or more amino acid alterations comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 or more alterations. An engineered ALDH described herein, in some embodiments, includes one or more amino acid alterations that are conservative or non-conservative amino acid substitutions, deletions, or insertions, or any combination thereof.

In some embodiments, an engineered ALDH described herein comprises one or more amino acid alterations at a position corresponding to position 2, 3, 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, 36, 37, 38, 39, 40, 41, 42, 43, 44, 45, 47, 48, 49, 50, 51, 52, 53, 54, 55, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 104, 106, 107, 108, 109, 110, 111, 112, 113, 114, 115, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, 201, 202, 203, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 243, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 288, 289, 290, 291, 292, 293, 294, 295, 296, 297, 298, 299, 300, 301, 302, 303, 304, 305, 306, 307, 308, 310, 311, 312, 313, 314, 315, 316, 317, 318, 319, 320, 321, 322, 323, 324, 325, 326, 327, 328, 329, 330, 331, 332, 333, 334, 335, 337, 338, 339, 341, 342, 343, 344, 345, 346, 347, 348, 349, 350, 351, 352, 354, 355, 356, 357, 358, 359, 360, 361, 362, 363, 364, 366, 368, 369, 370, 371, 372, 373, 374, 375, 376, 377, 378, 379, 380, 381, 382, 383, 384, 385, 386, 387, 388, 389, 390, 391, 392, 393, 394, 396, 397, 398, 399, 400, 401, 402, 403, 405, 406, 407, 408, 410, 411, 412, 413, 414, 415, 416, 418, 420, 421, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, or any combination thereof, in SEQ ID NO: 3.

In some embodiments, an engineered ALDH described herein comprises one or more amino acid alterations at a position corresponding to position 2, 3, 4, 5, 6, 7, 9, 11, 12, 15, 16, 17, 18, 20, 21, 22, 23, 24, 25, 26, 27, 28, 30, 31, 32, 33, 34, 35, 36, 37, 38, 39, 40, 41, 42, 43, 44, 45, 47, 48, 49, 51, 52, 54, 55, 58, 61, 62, 63, 64, 65, 66, 67, 70, 71, 73, 74, 75, 77, 78, 79, 80, 81, 82, 83, 84, 86, 88, 89, 90, 91, 92, 93, 94, 95, 96, 97, 98, 99, 100, 101, 102, 104, 107, 111, 117, 119, 120, 121, 122, 124, 125, 126, 128, 129, 130, 133, 136, 144, 145, 146, 147, 148, 149, 150, 151, 152, 153, 154, 155, 156, 157, 158, 159, 160, 162, 164, 165, 166, 167, 168, 169, 170, 171, 172, 174, 175, 176, 177, 180, 181, 183, 184, 187, 189, 190, 194, 196, 197, 198, 199, 200, 201, 202, 203, 206, 207, 208, 212, 214, 215, 216, 217, 218, 219, 221, 222, 223, 224, 225, 227, 228, 229, 230, 231, 232, 233, 234, 237, 238, 240, 243, 245, 246, 248, 249, 250, 251, 252, 253, 254, 255, 256, 257, 258, 259, 260, 261, 262, 263, 264, 265, 266, 267, 270, 271, 272, 273, 274, 275, 276, 277, 278, 279, 280, 281, 282, 283, 284, 285, 286, 288, 290, 291, 293, 294, 295, 296, 299, 300, 301, 302, 303, 304, 305, 306, 307, 308, 310, 311, 312, 313, 315, 316, 317, 318, 319, 320, 321, 322, 323, 324, 325, 326, 327, 328, 329, 330, 331, 332, 333, 334, 335, 339, 341, 342, 343, 344, 345, 346, 347, 348, 349, 350, 351, 354, 355, 356, 358, 359, 360, 362, 363, 366, 370, 371, 372, 373, 374, 375, 376, 377, 378, 379, 380, 381, 382, 383, 384, 385, 386, 387, 388, 389, 390, 392, 393, 394, 396, 397, 400, 401, 402, 405, 406, 407, 410, 411, 414, 420, 421, 423, 425, 426, 427, 428, 431, 433, 435, 436, 437, 438, 439, 440, 441, 443, 444, 446, 447, 448, 449, 450, 452, 453, 454, 455, 457, 458, 459 or a combination thereof, in SEQ ID NO: 3.

In some embodiments, an engineered ALDH described herein comprises one or more amino acid alterations at a position corresponding to position 42, 119, 149, 158, 174, 246, 259, 263, 299, 327, 348, 370, 392, 401, 427 or a combination thereof, in SEQ ID NO: 3.

In some embodiments, an engineered ALDH described herein having one or more amino acid alterations results in an engineered ALDH comprising: (a) P, Q, T at a residue corresponding to position 2 in SEQ ID NO: 3; (b) E, K, M, R, S at a residue corresponding to position 3 in SEQ ID NO: 3; (c) G, S at a residue corresponding to position 4 in SEQ ID NO: 3; (d) I, T at a residue corresponding to position 5 in SEQ ID NO: 3; (e) E, F, H, P, Q, R at a residue corresponding to position 6 in SEQ ID NO: 3; (f) E, Q at a residue corresponding to position 7 in SEQ ID NO: 3; (g) D at a residue corresponding to position 8 in SEQ ID NO: 3; (h) I, V at a residue corresponding to position 9 in SEQ ID NO: 3; (i) N at a residue corresponding to position 10 in SEQ ID NO: 3; (j) A, D, E, Q, R at a residue corresponding to position 11 in SEQ ID NO: 3; (k) A, I, L at a residue corresponding to position 12 in SEQ ID NO: 3; (l) L at a residue corresponding to position 13 in SEQ ID NO: 3; (m) D, E, K, Q at a residue corresponding to position 14 in SEQ ID NO: 3; (n) K, R at a residue corresponding to position 15 in SEQ ID NO: 3; (o) A at a residue corresponding to position 16 in SEQ ID NO: 3; (p) H, K, Q at a residue corresponding to position 17 in SEQ ID NO: 3; (q) E, K, Q, T at a residue corresponding to position 18 in SEQ ID NO: 3; (r) F, H, M at a residue corresponding to position 20 in SEQ ID NO: 3; (s) A, E, K, P, R at a residue corresponding to position 21 in SEQ ID NO: 3; (t) A, K, Q at a residue corresponding to position 22 in SEQ ID NO: 3; (u) L, W, Y at a residue corresponding to position 23 in SEQ ID NO: 3; (v) E, K, R, S at a residue corresponding to position 24 in SEQ ID NO: 3; (w) A, E, K, N, S at a residue corresponding to position 25 in SEQ ID NO: 3; (x) F, L, M, T, W at a residue corresponding to position 26 in SEQ ID NO: 3; (y) D, S, T at a residue corresponding to position 27 in SEQ ID NO: 3; (z) W at a residue corresponding to position 28 in SEQ ID NO: 3; (aa) K, Q at a residue corresponding to position 29 in SEQ ID NO: 3; (bb) D, E, K, Q, R at a residue corresponding to position 30 in SEQ ID NO: 3; (cc) I, L, R at a residue corresponding to position 31 in SEQ ID NO: 3; (dd) A, E, N at a residue corresponding to position 32 in SEQ ID NO: 3; (ee) A, E, Q, R at a residue corresponding to position 33 in SEQ ID NO: 3; (ff) I, L, M, V at a residue corresponding to position 34 in SEQ ID NO: 3; (gg) C, I, L, M at a residue corresponding to position 35 in SEQ ID NO: 3; (hh) E, H, K at a residue corresponding to position 36 in SEQ ID NO: 3; (ii) A, K, R at a residue corresponding to position 37 in SEQ ID NO: 3; (jj) F, I, L, M, W at a residue corresponding to position 38 in SEQ ID NO: 3; (kk) A, R at a residue corresponding to position 39 in SEQ ID NO: 3; (ll) D, E, Q, R, W at a residue corresponding to position 40 in SEQ ID NO: 3; (mm) L, V at a residue corresponding to position 41 in SEQ ID NO: 3; (nn) C, L, M, V at a residue corresponding to position 42 in SEQ ID NO: 3; (oo) E, L, R at a residue corresponding to position 43 in SEQ ID NO: 3; (pp) E, K, Q, R at a residue corresponding to position 44 in SEQ ID NO: 3; (qq) H, Q, R at a residue corresponding to position 45 in SEQ ID NO: 3; (rr) D at a residue corresponding to position 47 in SEQ ID NO: 3; (ss) D, E, M, R at a residue corresponding to position 48 in SEQ ID NO: 3; (tt) F, I, M, W, Y at a residue corresponding to position 49 in SEQ ID NO: 3; (uu) G at a residue corresponding to position 50 in SEQ ID NO: 3; (vv) A, E, K, N, Q, R at a residue corresponding to position 51 in SEQ ID NO: 3; (ww) L at a residue corresponding to position 52 in SEQ ID NO: 3; (xx) E, I, S at a residue corresponding to position 53 in SEQ ID NO: 3; (yy) I, L, M, T at a residue corresponding to position 54 in SEQ ID NO: 3; (zz) E, Q, R at a residue corresponding to position 55 in SEQ ID NO: 3; (aaa) E, G, Q, R at a residue corresponding to position 58 in SEQ ID NO: 3; (bbb) F, I, L, Y at a residue corresponding to position 59 in SEQ ID NO: 3; (ccc) A at a residue corresponding to position 60 in SEQ ID NO: 3; (ddd) D, E, K, R at a residue corresponding to position 61 in SEQ ID NO: 3; (eee) A, F, I, M, V, W at a residue corresponding to position 62 in SEQ ID NO: 3; (fff) D, E, Q at a residue corresponding to position 63 in SEQ ID NO: 3; (ggg) E, H at a residue corresponding to position 64 in SEQ ID NO: 3; (hhh) E, R at a residue corresponding to position 65 in SEQ ID NO: 3; (iii) L, V at a residue corresponding to position 66 in SEQ ID NO: 3; (jjj) E at a residue corresponding to position 67 in SEQ ID NO: 3; (kkk) D, E at a residue corresponding to position 68 in SEQ ID NO: 3; (lll) H, I, L, M, N at a residue corresponding to position 69 in SEQ ID NO: 3; (mmm) I, L, M, R at a residue corresponding to position 70 in SEQ ID NO: 3; (nnn) D, E, F, H, Y at a residue corresponding to position 71 in SEQ ID NO: 3; (ooo) A, R at a residue corresponding to position 72 in SEQ ID NO: 3; (ppp) A, T at a residue corresponding to position 73 in SEQ ID NO: 3; (qqq) D, E, R at a residue corresponding to position 74 in SEQ ID NO: 3; (rrr) G, P, Y at a residue corresponding to position 75 in SEQ ID NO: 3; (sss) F, I, L, M, Y at a residue corresponding to position 76 in SEQ ID NO: 3; (ttt) F, Y at a residue corresponding to position 77 in SEQ ID NO: 3; (uuu) E, F, H, N, R, Y at a residue corresponding to position 78 in SEQ ID NO: 3; (vvv) D, S, W, Y at a residue corresponding to position 79 in SEQ ID NO: 3; (www) F, I, L, V at a residue corresponding to position 80 in SEQ ID NO: 3; (xxx) A, E, R at a residue corresponding to position 81 in SEQ ID NO: 3; (yyy) E, G, P at a residue corresponding to position 82 in SEQ ID NO: 3; (zzz) E, Q at a residue corresponding to position 83 in SEQ ID NO: 3; (aaaa) Q, R at a residue corresponding to position 84 in SEQ ID NO: 3; (bbbb) A, T at a residue corresponding to position 85 in SEQ ID NO: 3; (cccc) E, K, V at a residue corresponding to position 86 in SEQ ID NO: 3; (dddd) T at a residue corresponding to position 87 in SEQ ID NO: 3; (eeee) L, M, V at a residue corresponding to position 88 in SEQ ID NO: 3; (ffff) L, P, V at a residue corresponding to position 89 in SEQ ID NO: 3; (gggg) D, E, H, P, R, S at a residue corresponding to position 90 in SEQ ID NO: 3; (hhhh) D, E, Q at a residue corresponding to position 91 in SEQ ID NO: 3; (iiii) D, M at a residue corresponding to position 92 in SEQ ID NO: 3; (jjjj) D, E at a residue corresponding to position 93 in SEQ ID NO: 3; (kkkk) A, D, G, N at a residue corresponding to position 94 in SEQ ID NO: 3; (llll) K, M, N, R at a residue corresponding to position 95 in SEQ ID NO: 3; (mmmm) H, K, N, R at a residue corresponding to position 96 in SEQ ID NO: 3; (nnnn) I, M, V at a residue corresponding to position 97 in SEQ ID NO: 3; (oooo) I, M, T, Y at a residue corresponding to position 98 in SEQ ID NO: 3; (pppp) K, Q, R, T at a residue corresponding to position 99 in SEQ ID NO: 3; (qqqq) Y, L, M, I at a residue corresponding to position 100 in SEQ ID NO: 3; (rrrr) K, Y, R, H at a residue corresponding to position 101 in SEQ ID NO: 3; (ssss) E, Q, R, V at a residue corresponding to position 102 in SEQ ID NO: 3; (tttt) V, Y, F, L, M at a residue corresponding to position 104 in SEQ ID NO: 3; (uuuu) I at a residue corresponding to position 106 in SEQ ID NO: 3; (vvvv) C, V at a residue corresponding to position 107 in SEQ ID NO: 3; (wwww) A, C at a residue corresponding to position 108 in SEQ ID NO: 3; (xxxx) I, A, M at a residue corresponding to position 109 in SEQ ID NO: 3; (yyyy) M, L, I at a residue corresponding to position 110 in SEQ ID NO: 3; (zzzz) T, V, Y, I, S at a residue corresponding to position 111 in SEQ ID NO: 3; (aaaaa) E at a residue corresponding to position 112 in SEQ ID NO: 3; (bbbbb) F, S, W, Y at a residue corresponding to position 113 in SEQ ID NO: 3; (ccccc) N at a residue corresponding to position 114 in SEQ ID NO: 3; (ddddd) W, R, H, Y at a residue corresponding to position 115 in SEQ ID NO: 3; (eeeee) N, L, T, I at a residue corresponding to position 117 in SEQ ID NO: 3; (fffff) N, A, L, M, S, F, V at a residue corresponding to position 118 in SEQ ID NO: 3; (ggggg) Q, I, L, M, T, S at a residue corresponding to position 119 in SEQ ID NO: 3; (hhhhh) T, A, P, M at a residue corresponding to position 120 in SEQ ID NO: 3; (iiiii) V, A, L, C, I at a residue corresponding to position 121 in SEQ ID NO: 3; (jjjjj) C, F, R, Y, H, N, S, G, V, W at a residue corresponding to position 122 in SEQ ID NO: 3; (kkkkk) K, P at a residue corresponding to position 123 in SEQ ID NO: 3; (lllll) V, L, M, I at a residue corresponding to position 124 in SEQ ID NO: 3; (mmmmm) I, L, A, V at a residue corresponding to position 125 in SEQ ID NO: 3; (nnnnn) A, P at a residue corresponding to position 126 in SEQ ID NO: 3; (ooooo) M at a residue corresponding to position 127 in SEQ ID NO: 3; (ppppp) I at a residue corresponding to position 128 in SEQ ID NO: 3; (qqqqq) A, M at a residue corresponding to position 129 in SEQ ID NO: 3; (rrrrr) T, S, A at a residue corresponding to position 130 in SEQ ID NO: 3; (sssss) R at a residue corresponding to position 131 in SEQ ID NO: 3; (ttttt) C at a residue corresponding to position 132 in SEQ ID NO: 3; (uuuuu) T, C at a residue corresponding to position 133 in SEQ ID NO: 3; (vvvvv) I, C, V at a residue corresponding to position 134 in SEQ ID NO: 3; (wwwww) V, M at a residue corresponding to position 135 in SEQ ID NO: 3; (xxxxx) L, M, F, I at a residue corresponding to position 136 in SEQ ID NO: 3; (yyyyy) R, K at a residue corresponding to position 137 in SEQ ID NO: 3; (zzzzz) G at a residue corresponding to position 138 in SEQ ID NO: 3; (aaaaaa) S, G at a residue corresponding to position 139 in SEQ ID NO: 3; (bbbbbb) E at a residue corresponding to position 140 in SEQ ID NO: 3; (cccccc) Q, K, Y, E at a residue corresponding to position 141 in SEQ ID NO: 3; (dddddd) T, S at a residue corresponding to position 142 in SEQ ID NO: 3; (eeeeee) P at a residue corresponding to position 143 in SEQ ID NO: 3; (ffffff) L, N, R at a residue corresponding to position 144 in SEQ ID NO: 3; (gggggg) T, C, V at a residue corresponding to position 145 in SEQ ID NO: 3; (hhhhhh) T, C, A, N at a residue corresponding to position 146 in SEQ ID NO: 3; (iiiiii) I, M, A at a residue corresponding to position 147 in SEQ ID NO: 3; (jjjjjj) Y, L, A, E, H, R at a residue corresponding to position 148 in SEQ ID NO: 3; (kkkkkk) V, I, L, M, F at a residue corresponding to position 149 in SEQ ID NO: 3; (llllll) I, M, G, A at a residue corresponding to position 150 in SEQ ID NO: 3; (mmmmmm) D, K, R, Q at a residue corresponding to position 151 in SEQ ID NO: 3; (nnnnnn) L, M, I at a residue corresponding to position 152 in SEQ ID NO: 3; (oooooo) F, V, C, M at a residue corresponding to position 153 in SEQ ID NO: 3; (pppppp) H, A, Q, K, R at a residue corresponding to position 154 in SEQ ID NO: 3; (qqqqqq) R, K, D, Q at a residue corresponding to position 155 in SEQ ID NO: 3; (rrrrrr) I at a residue corresponding to position 156 in SEQ ID NO: 3; (ssssss) A, I, F at a residue corresponding to position 157 in SEQ ID NO: 3; (tttttt) E, V, K at a residue corresponding to position 158 in SEQ ID NO: 3; (uuuuuu) K, Q, D, E at a residue corresponding to position 159 in SEQ ID NO: 3; (vvvvvv) V, C, A at a residue corresponding to position 160 in SEQ ID NO: 3; (wwwwww) I, A, F, Y, L, V at a residue corresponding to position 162 in SEQ ID NO: 3; (xxxxxx) D, A, P at a residue corresponding to position 164 in SEQ ID NO: 3; (yyyyyy) H, G, D at a residue corresponding to position 165 in SEQ ID NO: 3; (zzzzzz) C, A, I, V at a residue corresponding to position 166 in SEQ ID NO: 3; (aaaaaaa) V, F, L at a residue corresponding to position 167 in SEQ ID NO: 3; (bbbbbbb) T, N at a residue corresponding to position 168 in SEQ ID NO: 3; (ccccccc) L, W, C, M, I at a residue corresponding to position 169 in SEQ ID NO: 3; (ddddddd) L, V at a residue corresponding to position 170 in SEQ ID NO: 3; (eeeeeee) E, P, N, D at a residue corresponding to position 171 in SEQ ID NO: 3; (fffffff) D, G, H at a residue corresponding to position 172 in SEQ ID NO: 3; (ggggggg) G, D at a residue corresponding to position 173 in SEQ ID NO: 3; (hhhhhhh) G, D, T at a residue corresponding to position 174 in SEQ ID NO: 3; (iiiiiii) I, H, R at a residue corresponding to position 175 in SEQ ID NO: 3; (jjjjjjj) Q, D at a residue corresponding to position 176 in SEQ ID NO: 3; (kkkkkkk) V, T, A, I at a residue corresponding to position 177 in SEQ ID NO: 3; (lllllll) V, A, T, G at a residue corresponding to position 178 in SEQ ID NO: 3; (mmmmmmm) D, N, E at a residue corresponding to position 179 in SEQ ID NO: 3; (nnnnnnn) H, Q, A, E at a residue corresponding to position 180 in SEQ ID NO: 3; (ooooooo) I, F at a residue corresponding to position 181 in SEQ ID NO: 3; (ppppppp) L, H, I, V at a residue corresponding to position 182 in SEQ ID NO: 3; (qqqqqqq) Q, K, D, A, H, E at a residue corresponding to position 183 in SEQ ID NO: 3; (rrrrrrr) H, P, S, G at a residue corresponding to position 184 in SEQ ID NO: 3; (sssssss) C, I at a residue corresponding to position 185 in SEQ ID NO: 3; (ttttttt) R at a residue corresponding to position 186 in SEQ ID NO: 3; (uuuuuuu) H, K, F, I, V, M at a residue corresponding to position 187 in SEQ ID NO: 3; (vvvvvvv) I, L, V at a residue corresponding to position 188 in SEQ ID NO: 3; (wwwwwww) S, F, T, M, V, L at a residue corresponding to position 189 in SEQ ID NO: 3; (xxxxxxx) P, A, F at a residue corresponding to position 190 in SEQ ID NO: 3; (yyyyyyy) R at a residue corresponding to position 191 in SEQ ID NO: 3; (zzzzzzz) S at a residue corresponding to position 193 in SEQ ID NO: 3; (aaaaaaaa) T, R, K, A at a residue corresponding to position 194 in SEQ ID NO: 3; (bbbbbbbb) A at a residue corresponding to position 195 in SEQ ID NO: 3; (cccccccc) M, I, V at a residue corresponding to position 196 in SEQ ID NO: 3; (dddddddd) I, M, G, A at a residue corresponding to position 197 in SEQ ID NO: 3; (eeeeeeee) Q, E, R at a residue corresponding to position 198 in SEQ ID NO: 3; (ffffffff) S, V at a residue corresponding to position 199 in SEQ ID NO: 3; (gggggggg) C, I, V at a residue corresponding to position 200 in SEQ ID NO: 3; (hhhhhhhh) M, Q, A at a residue corresponding to position 201 in SEQ ID NO: 3; (iiiiiiii) N, K, A, R, T, E, Q at a residue corresponding to position 202 in SEQ ID NO: 3; (jjjjjjjj) N, H, A at a residue corresponding to position 203 in SEQ ID NO: 3; (kkkkkkkk) R, T at a residue corresponding to position 205 in SEQ ID NO: 3; (llllllll) V, R, K at a residue corresponding to position 206 in SEQ ID NO: 3; (mmmmmmmm) V, S, C, T at a residue corresponding to position 207 in SEQ ID NO: 3; (nnnnnnnn) V, T, Y, M, L at a residue corresponding to position 208 in SEQ ID NO: 3; (oooooooo) L, E, M at a residue corresponding to position 209 in SEQ ID NO: 3; (pppppppp) H, L at a residue corresponding to position 210 in SEQ ID NO: 3; (qqqqqqqq) L at a residue corresponding to position 211 in SEQ ID NO: 3; (rrrrrrrr) A, P, G at a residue corresponding to position 212 in SEQ ID NO: 3; (ssssssss) K at a residue corresponding to position 213 in SEQ ID NO: 3; (tttttttt) S, K at a residue corresponding to position 214 in SEQ ID NO: 3; (uuuuuuuu) S, N, A, C, D, P at a residue corresponding to position 215 in SEQ ID NO: 3; (vvvvvvvv) H, A, P at a residue corresponding to position 216 in SEQ ID NO: 3; (wwwwwwww) F, A, I, C, M at a residue corresponding to position 217 in SEQ ID NO: 3; (xxxxxxxx) I, V, F, Y at a residue corresponding to position 218 in SEQ ID NO: 3; (yyyyyyyy) M, F, L, I at a residue corresponding to position 219 in SEQ ID NO: 3; (zzzzzzzz) C, F at a residue corresponding to position 220 in SEQ ID NO: 3; (aaaaaaaaa) D, P at a residue corresponding to position 221 in SEQ ID NO: 3; (bbbbbbbbb) T, S at a residue corresponding to position 222 in SEQ ID NO: 3; (ccccccccc) C at a residue corresponding to position 223 in SEQ ID NO: 3; (ddddddddd) N at a residue corresponding to position 224 in SEQ ID NO: 3; (eeeeeeeee) V at a residue corresponding to position 225 in SEQ ID NO: 3; (fffffffff) E at a residue corresponding to position 226 in SEQ ID NO: 3; (ggggggggg) K, R, M, E at a residue corresponding to position 227 in SEQ ID NO: 3; (hhhhhhhhh) A at a residue corresponding to position 228 in SEQ ID NO: 3; (iiiiiiiii) W, V at a residue corresponding to position 229 in SEQ ID NO: 3; (jjjjjjjjj) R, D, P, Q, K, E at a residue corresponding to position 230 in SEQ ID NO: 3; (kkkkkkkkk) Q, R, I, K at a residue corresponding to position 231 in SEQ ID NO: 3; (lllllllll) I, F, T, C, Q, M, N at a residue corresponding to position 232 in SEQ ID NO: 3; (mmmmmmmmm) V, L, M at a residue corresponding to position 233 in SEQ ID NO: 3; (nnnnnnnnn) W, Q, F, A, M at a residue corresponding to position 234 in SEQ ID NO: 3; (ooooooooo) A, S at a residue corresponding to position 235 in SEQ ID NO: 3; (ppppppppp) K, C, W, A, I at a residue corresponding to position 236 in SEQ ID NO: 3; (qqqqqqqqq) F, Y, C, M at a residue corresponding to position 237 in SEQ ID NO: 3; (rrrrrrrrr) R, M, Q, Y, W at a residue corresponding to position 238 in SEQ ID NO: 3; (sssssssss) R, H, Q at a residue corresponding to position 239 in SEQ ID NO: 3; (ttttttttt) Q, A, T, C, S at a residue corresponding to position 240 in SEQ ID NO: 3; (uuuuuuuuu) N at a residue corresponding to position 241 in SEQ ID NO: 3; (vvvvvvvvv) V, M, T, C at a residue corresponding to position 243 in SEQ ID NO: 3; (wwwwwwwww) M, I, N, S, V, A, C at a residue corresponding to position 245 in SEQ ID NO: 3; (xxxxxxxxx) S, A at a residue corresponding to position 246 in SEQ ID NO: 3; (yyyyyyyyy) G, P, I, A at a residue corresponding to position 247 in SEQ ID NO: 3; (zzzzzzzzz) D, T, E, K, S, N at a residue corresponding to position 248 in SEQ ID NO: 3; (aaaaaaaaaa) Y, R at a residue corresponding to position 249 in SEQ ID NO: 3; (bbbbbbbbbb) I, L, M at a residue corresponding to position 250 in SEQ ID NO: 3; (cccccccccc) Y, F, L, V at a residue corresponding to position 251 in SEQ ID NO: 3; (dddddddddd) V, I at a residue corresponding to position 252 in SEQ ID NO: 3; (eeeeeeeeee) D, Q, H, V, E at a residue corresponding to position 253 in SEQ ID NO: 3; (ffffffffff) Q, K, R, D at a residue corresponding to position 254 in SEQ ID NO: 3; (gggggggggg) E at a residue corresponding to position 255 in SEQ ID NO: 3; (hhhhhhhhhh) V, M, I, R at a residue corresponding to position 256 in SEQ ID NO: 3; (iiiiiiiiii) A, F, H, Y at a residue corresponding to position 257 in SEQ ID NO: 3; (jjjjjjjjjj) D at a residue corresponding to position 258 in SEQ ID NO: 3; (kkkkkkkkkk) A, R, K, D, Q at a residue corresponding to position 259 in SEQ ID NO: 3; (llllllllll) I, V, F, L at a residue corresponding to position 260 in SEQ ID NO: 3; (mmmmmmmmmm) L, M, V at a residue corresponding to position 261 in SEQ ID NO: 3; (nnnnnnnnnn) Q, D, E, K at a residue corresponding to position 262 in SEQ ID NO: 3; (oooooooooo) E, K, R at a residue corresponding to position 263 in SEQ ID NO: 3; (pppppppppp) M, V, F at a residue corresponding to position 264 in SEQ ID NO: 3; (qqqqqqqqqq) Q, R, A, E at a residue corresponding to position 265 in SEQ ID NO: 3; (rrrrrrrrrr) A, E at a residue corresponding to position 266 in SEQ ID NO: 3; (ssssssssss) Y, R, N, A, Q at a residue corresponding to position 267 in SEQ ID NO: 3; (tttttttttt) A at a residue corresponding to position 268 in SEQ ID NO: 3; (uuuuuuuuuu) S, G, C at a residue corresponding to position 269 in SEQ ID NO: 3; (vvvvvvvvvv) L, F, I, M, W at a residue corresponding to position 270 in SEQ ID NO: 3; (wwwwwwwwww) F, K, V, W, R, H at a residue corresponding to position 271 in SEQ ID NO: 3; (xxxxxxxxxx) V, M, L at a residue corresponding to position 272 in SEQ ID NO: 3; (yyyyyyyyyy) C at a residue corresponding to position 273 in SEQ ID NO: 3; (zzzzzzzzzz) E, D at a residue corresponding to position 274 in SEQ ID NO: 3; (aaaaaaaaaaa) P, G, A, E at a residue corresponding to position 275 in SEQ ID NO: 3; (bbbbbbbbbbb) Q, M, R, D at a residue corresponding to position 276 in SEQ ID NO: 3; (ccccccccccc) Q, E at a residue corresponding to position 277 in SEQ ID NO: 3; (ddddddddddd) P, E at a residue corresponding to position 278 in SEQ ID NO: 3; (eeeeeeeeeee) E, D, S at a residue corresponding to position 279 in SEQ ID NO: 3; (fffffffffff) Q, R, T at a residue corresponding to position 280 in SEQ ID NO: 3; (ggggggggggg) V, M, I at a residue corresponding to position 281 in SEQ ID NO: 3; (hhhhhhhhhhh) Q, K, M, G, E at a residue corresponding to position 282 in SEQ ID NO: 3; (iiiiiiiiiii) E, H, N at a residue corresponding to position 283 in SEQ ID NO: 3; (jjjjjjjjjjj) V, P at a residue corresponding to position 284 in SEQ ID NO: 3; (kkkkkkkkkkk) L, V, I, M at a residue corresponding to position 285 in SEQ ID NO: 3; (lllllllllll) L, I, W, Y, V at a residue corresponding to position 286 in SEQ ID NO: 3; (mmmmmmmmmmm) R, D, E at a residue corresponding to position 288 in SEQ ID NO: 3; (nnnnnnnnnnn) M at a residue corresponding to position 289 in SEQ ID NO: 3; (ooooooooooo) H at a residue corresponding to position 290 in SEQ ID NO: 3; (ppppppppppp) K, L, E, T, H, A, N at a residue corresponding to position 291 in SEQ ID NO: 3; (qqqqqqqqqqq) I, M at a residue corresponding to position 292 in SEQ ID NO: 3; (rrrrrrrrrrr) A, V, I, N at a residue corresponding to position 293 in SEQ ID NO: 3; (sssssssssss) S, N, P, E, R at a residue corresponding to position 294 in SEQ ID NO: 3; (ttttttttttt) R, K, A, Q, D at a residue corresponding to position 295 in SEQ ID NO: 3; (uuuuuuuuuuu) W, R, V, F, H, L at a residue corresponding to position 296 in SEQ ID NO: 3; (vvvvvvvvvvv) A, T, Q, V, L, H, I at a residue corresponding to position 297 in SEQ ID NO: 3; (wwwwwwwwwww) A at a residue corresponding to position 298 in SEQ ID NO: 3; (xxxxxxxxxxx) K, Q, R at a residue corresponding to position 299 in SEQ ID NO: 3; (yyyyyyyyyyy) A, R at a residue corresponding to position 300 in SEQ ID NO: 3; (zzzzzzzzzzz) I, A at a residue corresponding to position 301 in SEQ ID NO: 3; (aaaaaaaaaaaa) A, R, D, Q, E at a residue corresponding to position 302 in SEQ ID NO: 3; (bbbbbbbbbbbb) Q, W, R, E at a residue corresponding to position 303 in SEQ ID NO: 3; (cccccccccccc) L, G, Y at a residue corresponding to position 304 in SEQ ID NO: 3; (dddddddddddd) I, V at a residue corresponding to position 305 in SEQ ID NO: 3; (eeeeeeeeeeee) H, K, E, R at a residue corresponding to position 306 in SEQ ID NO: 3; (ffffffffffff) I, V, M at a residue corresponding to position 307 in SEQ ID NO: 3; (gggggggggggg) A, T at a residue corresponding to position 308 in SEQ ID NO: 3; (hhhhhhhhhhhh) G, V, L, F at a residue corresponding to position 310 in SEQ ID NO: 3; (iiiiiiiiiiii) R, T, K, S, D, E at a residue corresponding to position 311 in SEQ ID NO: 3; (jjjjjjjjjjjj) A, D, T, K at a residue corresponding to position 312 in SEQ ID NO: 3; (kkkkkkkkkkkk) L, V at a residue corresponding to position 313 in SEQ ID NO: 3; (llllllllllll) A at a residue corresponding to position 314 in SEQ ID NO: 3; (mmmmmmmmmmmm) D at a residue corresponding to position 315 in SEQ ID NO: 3; (nnnnnnnnnnnn) E, G, N at a residue corresponding to position 316 in SEQ ID NO: 3; (oooooooooooo) G, T at a residue corresponding to position 317 in SEQ ID NO: 3; (pppppppppppp) R, T, Y at a residue corresponding to position 318 in SEQ ID NO: 3; (qqqqqqqqqqqq) F, L, M, I, Y at a residue corresponding to position 319 in SEQ ID NO: 3; (rrrrrrrrrrrr) Y, L at a residue corresponding to position 320 in SEQ ID NO: 3; (ssssssssssss) L, P, C, I at a residue corresponding to position 321 in SEQ ID NO: 3; (tttttttttttt) A, T, G, V at a residue corresponding to position 322 in SEQ ID NO: 3; (uuuuuuuuuuuu) E, L, I, G, P, V, K at a residue corresponding to position 323 in SEQ ID NO: 3; (vvvvvvvvvvvv) I, V, L at a residue corresponding to position 324 in SEQ ID NO: 3; (wwwwwwwwwwww) K, E, D, T at a residue corresponding to position 325 in SEQ ID NO: 3; (xxxxxxxxxxxx) D at a residue corresponding to position 326 in SEQ ID NO: 3; (yyyyyyyyyyyy) W, F, V at a residue corresponding to position 327 in SEQ ID NO: 3; (zzzzzzzzzzzz) D, T, S at a residue corresponding to position 328 in SEQ ID NO: 3; (aaaaaaaaaaaaa) E, R, P, D, N at a residue corresponding to position 329 in SEQ ID NO: 3; (bbbbbbbbbbbbb) E at a residue corresponding to position 330 in SEQ ID NO: 3; (ccccccccccccc) S, D, H, M, Y at a residue corresponding to position 331 in SEQ ID NO: 3; (ddddddddddddd) R, T at a residue corresponding to position 332 in SEQ ID NO: 3; (eeeeeeeeeeeee) M, V, F, I at a residue corresponding to position 333 in SEQ ID NO: 3; (fffffffffffff) V, S, M, C at a residue corresponding to position 334 in SEQ ID NO: 3; (ggggggggggggg) R, Q, H at a residue corresponding to position 335 in SEQ ID NO: 3; (hhhhhhhhhhhhh) Q, E at a residue corresponding to position 337 in SEQ ID NO: 3; (iiiiiiiiiiiii) F, V, I, M at a residue corresponding to position 338 in SEQ ID NO: 3; (jjjjjjjjjjjjj) C, M at a residue corresponding to position 339 in SEQ ID NO: 3; (kkkkkkkkkkkkk) I at a residue corresponding to position 341 in SEQ ID NO: 3; (lllllllllllll) M, V, I at a residue corresponding to position 342 in SEQ ID NO: 3; (mmmmmmmmmmmmm) C, T, V, S, P at a residue corresponding to position 343 in SEQ ID NO: 3; (nnnnnnnnnnnnn) I, V, L, M at a residue corresponding to position 344 in SEQ ID NO: 3; (ooooooooooooo) Y, V, M, L, I at a residue corresponding to position 345 in SEQ ID NO: 3; (ppppppppppppp) R, K, P at a residue corresponding to position 346 in SEQ ID NO: 3; (qqqqqqqqqqqqq) F, V at a residue corresponding to position 347 in SEQ ID NO: 3; (rrrrrrrrrrrrr) E, K, S at a residue corresponding to position 348 in SEQ ID NO: 3; (sssssssssssss) S, G, D, N, T at a residue corresponding to position 349 in SEQ ID NO: 3; (ttttttttttttt) V, E, I, L, F at a residue corresponding to position 350 in SEQ ID NO: 3; (uuuuuuuuuuuuu) D at a residue corresponding to position 351 in SEQ ID NO: 3; (vvvvvvvvvvvvv) Q, H, E at a residue corresponding to position 352 in SEQ ID NO: 3; (wwwwwwwwwwwww) L, V, C at a residue corresponding to position 354 in SEQ ID NO: 3; (xxxxxxxxxxxxx) R, A, D, Q, S, E at a residue corresponding to position 355 in SEQ ID NO: 3; (yyyyyyyyyyyyy) M, L, V at a residue corresponding to position 356 in SEQ ID NO: 3; (zzzzzzzzzzzzz) C at a residue corresponding to position 357 in SEQ ID NO: 3; (aaaaaaaaaaaaaa) N, R at a residue corresponding to position 358 in SEQ ID NO: 3; (bbbbbbbbbbbbbb) A, D, S, N, E at a residue corresponding to position 359 in SEQ ID NO: 3; (cccccccccccccc) S, T at a residue corresponding to position 360 in SEQ ID NO: 3; (dddddddddddddd) D, V at a residue corresponding to position 361 in SEQ ID NO: 3; (eeeeeeeeeeeeee) L, T, N, S at a residue corresponding to position 362 in SEQ ID NO: 3; (ffffffffffffff) Y, E at a residue corresponding to position 363 in SEQ ID NO: 3; (gggggggggggggg) Q, Y, F, H at a residue corresponding to position 364 in SEQ ID NO: 3; (hhhhhhhhhhhhhh) Y, K, F, A, L at a residue corresponding to position 366 in SEQ ID NO: 3; (iiiiiiiiiiiiii) L, A at a residue corresponding to position 368 in SEQ ID NO: 3; (jjjjjjjjjjjjjj) T, A at a residue corresponding to position 369 in SEQ ID NO: 3; (kkkkkkkkkkkkkk) C, A at a residue corresponding to position 370 in SEQ ID NO: 3; (llllllllllllll) M, I, A, W, S, Y at a residue corresponding to position 371 in SEQ ID NO: 3; (mmmmmmmmmmmmmm) V, M at a residue corresponding to position 372 in SEQ ID NO: 3; (nnnnnnnnnnnnnn) W, Y, F at a residue corresponding to position 373 in SEQ ID NO: 3; (oooooooooooooo) T at a residue corresponding to position 374 in SEQ ID NO: 3; (pppppppppppppp) H, Q, K, T, R at a residue corresponding to position 375 in SEQ ID NO: 3; (qqqqqqqqqqqqqq) N, S, D at a residue corresponding to position 376 in SEQ ID NO: 3; (rrrrrrrrrrrrrr) D, I, V, E at a residue corresponding to position 377 in SEQ ID NO: 3; (ssssssssssssss) K, D, A at a residue corresponding to position 378 in SEQ ID NO: 3; (tttttttttttttt) N, K, V, R at a residue corresponding to position 379 in SEQ ID NO: 3; (uuuuuuuuuuuuuu) A, M, V at a residue corresponding to position 380 in SEQ ID NO: 3; (vvvvvvvvvvvvvv) M, W, K, R, T, H at a residue corresponding to position 381 in SEQ ID NO: 3; (wwwwwwwwwwwwww) K, R, E, Q at a residue corresponding to position 382 in SEQ ID NO: 3; (xxxxxxxxxxxxxx) M, L, Y, F, I at a residue corresponding to position 383 in SEQ ID NO: 3; (yyyyyyyyyyyyyy) S, G at a residue corresponding to position 384 in SEQ ID NO: 3; (zzzzzzzzzzzzzz) Q, L, R, K at a residue corresponding to position 385 in SEQ ID NO: 3; (aaaaaaaaaaaaaaa) E, Q, K, R at a residue corresponding to position 386 in SEQ ID NO: 3; (bbbbbbbbbbbbbbb) M, V, L at a residue corresponding to position 387 in SEQ ID NO: 3; (ccccccccccccccc) R, H, D, Q, E at a residue corresponding to position 388 in SEQ ID NO: 3; (ddddddddddddddd) A, C, T at a residue corresponding to position 389 in SEQ ID NO: 3; (eeeeeeeeeeeeeee) T, N, C, G at a residue corresponding to position 390 in SEQ ID NO: 3; (fffffffffffffff) M, T, I at a residue corresponding to position 391 in SEQ ID NO: 3; (ggggggggggggggg) V, I, M at a residue corresponding to position 392 in SEQ ID NO: 3; (hhhhhhhhhhhhhhh) M, I, W, L, G at a residue corresponding to position 393 in SEQ ID NO: 3; (iiiiiiiiiiiiiii) I, K at a residue corresponding to position 394 in SEQ ID NO: 3; (jjjjjjjjjjjjjjj) R, E, S, D at a residue corresponding to position 396 in SEQ ID NO: 3; (kkkkkkkkkkkkkkk) I, W, A, P, F, Y at a residue corresponding to position 397 in SEQ ID NO: 3; (lllllllllllllll) S, C, P, T at a residue corresponding to position 398 in SEQ ID NO: 3; (mmmmmmmmmmmmmmm) A, S at a residue corresponding to position 399 in SEQ ID NO: 3; (nnnnnnnnnnnnnnn) I, S, F, V, H at a residue corresponding to position 400 in SEQ ID NO: 3; (ooooooooooooooo) G, H, D, S, A at a residue corresponding to position 401 in SEQ ID NO: 3; (ppppppppppppppp) N, A, F, M at a residue corresponding to position 402 in SEQ ID NO: 3; (qqqqqqqqqqqqqqq) S at a residue corresponding to position 403 in SEQ ID NO: 3; (rrrrrrrrrrrrrrr) I, F, V, G at a residue corresponding to position 405 in SEQ ID NO: 3; (sssssssssssssss) Y, K, F, L, W at a residue corresponding to position 406 in SEQ ID NO: 3; (ttttttttttttttt) H, Q, Y, E at a residue corresponding to position 407 in SEQ ID NO: 3; (uuuuuuuuuuuuuuu) T, S, H at a residue corresponding to position 408 in SEQ ID NO: 3; (vvvvvvvvvvvvvvv) F, I, M at a residue corresponding to position 410 in SEQ ID NO: 3; (wwwwwwwwwwwwwww) T, G, P, A at a residue corresponding to position 411 in SEQ ID NO: 3; (xxxxxxxxxxxxxxx) F, R at a residue corresponding to position 412 in SEQ ID NO: 3; (yyyyyyyyyyyyyyy) A at a residue corresponding to position 413 in SEQ ID NO: 3; (zzzzzzzzzzzzzzz) M, F, V at a residue corresponding to position 414 in SEQ ID NO: 3; (aaaaaaaaaaaaaaaa) P, S at a residue corresponding to position 415 in SEQ ID NO: 3; (bbbbbbbbbbbbbbbb) M, F, I at a residue corresponding to position 416 in SEQ ID NO: 3; (cccccccccccccccc) F, I, T at a residue corresponding to position 418 in SEQ ID NO: 3; (dddddddddddddddd) S, R at a residue corresponding to position 420 in SEQ ID NO: 3; (eeeeeeeeeeeeeeee) Y, F at a residue corresponding to position 421 in SEQ ID NO: 3; (ffffffffffffffff) K, R, G at a residue corresponding to position 423 in SEQ ID NO: 3; (gggggggggggggggg) S, R at a residue corresponding to position 424 in SEQ ID NO: 3; (hhhhhhhhhhhhhhhh) I, G, W, A at a residue corresponding to position 425 in SEQ ID NO: 3; (iiiiiiiiiiiiiiii) V, T, L at a residue corresponding to position 426 in SEQ ID NO: 3; (jjjjjjjjjjjjjjjj) G, T, A at a residue corresponding to position 427 in SEQ ID NO: 3; (kkkkkkkkkkkkkkkk) D, H at a residue corresponding to position 428 in SEQ ID NO: 3; (llllllllllllllll) E at a residue corresponding to position 429 in SEQ ID NO: 3; (mmmmmmmmmmmmmmmm) I, Y, L, V at a residue corresponding to position 430 in SEQ ID NO: 3; (nnnnnnnnnnnnnnnn) G, S, N at a residue corresponding to position 431 in SEQ ID NO: 3; (oooooooooooooooo) A, P, F, W at a residue corresponding to position 432 in SEQ ID NO: 3; (pppppppppppppppp) T, R, K, H, M, E at a residue corresponding to position 433 in SEQ ID NO: 3; (qqqqqqqqqqqqqqqq) R, Q, D, N at a residue corresponding to position 434 in SEQ ID NO: 3; (rrrrrrrrrrrrrrrr) V, F, W, Y, M at a residue corresponding to position 435 in SEQ ID NO: 3; (ssssssssssssssss) T, L, F, I, V at a residue corresponding to position 436 in SEQ ID NO: 3; (tttttttttttttttt) H, Q, Y at a residue corresponding to position 437 in SEQ ID NO: 3; (uuuuuuuuuuuuuuuu) V, T, W, C, Y, I at a residue corresponding to position 438 in SEQ ID NO: 3; (vvvvvvvvvvvvvvvv) Q, S, R, K at a residue corresponding to position 439 in SEQ ID NO: 3; (wwwwwwwwwwwwwwww) K, W at a residue corresponding to position 440 in SEQ ID NO: 3; (xxxxxxxxxxxxxxxx) L, I, M at a residue corresponding to position 441 in SEQ ID NO: 3; (yyyyyyyyyyyyyyyy) G, A at a residue corresponding to position 442 in SEQ ID NO: 3; (zzzzzzzzzzzzzzzz) H, W, R, F at a residue corresponding to position 443 in SEQ ID NO: 3; (aaaaaaaaaaaaaaaaa) E, P, K at a residue corresponding to position 444 in SEQ ID NO: 3; (bbbbbbbbbbbbbbbbb) I, Y, F, M, L at a residue corresponding to position 445 in SEQ ID NO: 3; (ccccccccccccccccc) E, K at a residue corresponding to position 446 in SEQ ID NO: 3; (ddddddddddddddddd) E, N at a residue corresponding to position 447 in SEQ ID NO: 3; (eeeeeeeeeeeeeeeee) W, K, P at a residue corresponding to position 448 in SEQ ID NO: 3; (fffffffffffffffff) E, R, F, H at a residue corresponding to position 449 in SEQ ID NO: 3; (ggggggggggggggggg) M, C, F, V, L at a residue corresponding to position 450 in SEQ ID NO: 3; (hhhhhhhhhhhhhhhhh) W at a residue corresponding to position 451 in SEQ ID NO: 3; (iiiiiiiiiiiiiiiii) S, D, K at a residue corresponding to position 452 in SEQ ID NO: 3; (jjjjjjjjjjjjjjjjj) E, P, H at a residue corresponding to position 453 in SEQ ID NO: 3; (kkkkkkkkkkkkkkkkk) A, D at a residue corresponding to position 454 in SEQ ID NO: 3; (lllllllllllllllll) K, Q, D at a residue corresponding to position 455 in SEQ ID NO: 3; (mmmmmmmmmmmmmmmmm) F, L, V, W, M at a residue corresponding to position 456 in SEQ ID NO: 3; (nnnnnnnnnnnnnnnnn) M, Y, F at a residue corresponding to position 457 in SEQ ID NO: 3; (ooooooooooooooooo) H, G at a residue corresponding to position 458 in SEQ ID NO: 3; (ppppppppppppppppp) D at a residue corresponding to position 459 in SEQ ID NO: 3; or (qqqqqqqqqqqqqqqqq) Any combination thereof.

In some embodiments, an engineered ALDH described herein having one or more amino acid alterations results in an engineered ALDH comprising: (a) Q, T, P at a residue corresponding to position 2 in SEQ ID NO: 3; (b) S, K at a residue corresponding to position 3 in SEQ ID NO: 3; (c) G, S at a residue corresponding to position 4 in SEQ ID NO: 3; (d) I, T at a residue corresponding to position 5 in SEQ ID NO: 3; (e) F at a residue corresponding to position 6 in SEQ ID NO: 3; (f) E, Q at a residue corresponding to position 7 in SEQ ID NO: 3; (g) V at a residue corresponding to position 9 in SEQ ID NO: 3; (h) E, D, Q, R at a residue corresponding to position 11 in SEQ ID NO: 3; (i) L, A at a residue corresponding to position 12 in SEQ ID NO: 3; (j) R, K at a residue corresponding to position 15 in SEQ ID NO: 3; (k) A at a residue corresponding to position 16 in SEQ ID NO: 3; (l) K at a residue corresponding to position 17 in SEQ ID NO: 3; (m) K, E, Q at a residue corresponding to position 18 in SEQ ID NO: 3; (n) F, H at a residue corresponding to position 20 in SEQ ID NO: 3; (o) A, R at a residue corresponding to position 21 in SEQ ID NO: 3; (p) K, Q, A at a residue corresponding to position 22 in SEQ ID NO: 3; (q) Y at a residue corresponding to position 23 in SEQ ID NO: 3; (r) E, K at a residue corresponding to position 24 in SEQ ID NO: 3; (s) A, E at a residue corresponding to position 25 in SEQ ID NO: 3; (t) L, M, W at a residue corresponding to position 26 in SEQ ID NO: 3; (u) D, T at a residue corresponding to position 27 in SEQ ID NO: 3; (v) W at a residue corresponding to position 28 in SEQ ID NO: 3; (w) D, K, Q at a residue corresponding to position 30 in SEQ ID NO: 3; (x) I, L at a residue corresponding to position 31 in SEQ ID NO: 3; (y) N, E at a residue corresponding to position 32 in SEQ ID NO: 3; (z) E, Q, A, R at a residue corresponding to position 33 in SEQ ID NO: 3; (aa) V at a residue corresponding to position 34 in SEQ ID NO: 3; (bb) C, M at a residue corresponding to position 35 in SEQ ID NO: 3; (cc) K at a residue corresponding to position 36 in SEQ ID NO: 3; (dd) K, A, R at a residue corresponding to position 37 in SEQ ID NO: 3; (ee) L, I at a residue corresponding to position 38 in SEQ ID NO: 3; (ff) A at a residue corresponding to position 39 in SEQ ID NO: 3; (gg) W at a residue corresponding to position 40 in SEQ ID NO: 3; (hh) L, V at a residue corresponding to position 41 in SEQ ID NO: 3; (ii) L, V, M, C at a residue corresponding to position 42 in SEQ ID NO: 3; (jj) E, L at a residue corresponding to position 43 in SEQ ID NO: 3; (kk) K, Q at a residue corresponding to position 44 in SEQ ID NO: 3; (ll) H, R at a residue corresponding to position 45 in SEQ ID NO: 3; (mm) D at a residue corresponding to position 47 in SEQ ID NO: 3; (nn) E, M, D at a residue corresponding to position 48 in SEQ ID NO: 3; (oo) I at a residue corresponding to position 49 in SEQ ID NO: 3; (pp) N, E, Q at a residue corresponding to position 51 in SEQ ID NO: 3; (qq) L at a residue corresponding to position 52 in SEQ ID NO: 3; (rr) L at a residue corresponding to position 54 in SEQ ID NO: 3; (ss) E, R at a residue corresponding to position 55 in SEQ ID NO: 3; (tt) R at a residue corresponding to position 58 in SEQ ID NO: 3; (uu) D, E, R at a residue corresponding to position 61 in SEQ ID NO: 3; (vv) F, V, W at a residue corresponding to position 62 in SEQ ID NO: 3; (ww) D, E, Q at a residue corresponding to position 63 in SEQ ID NO: 3; (xx) H at a residue corresponding to position 64 in SEQ ID NO: 3; (yy) R at a residue corresponding to position 65 in SEQ ID NO: 3; (zz) V at a residue corresponding to position 66 in SEQ ID NO: 3; (aaa) E at a residue corresponding to position 67 in SEQ ID NO: 3; (bbb) M, R at a residue corresponding to position 70 in SEQ ID NO: 3; (ccc) D, E, Y, F, H at a residue corresponding to position 71 in SEQ ID NO: 3; (ddd) T at a residue corresponding to position 73 in SEQ ID NO: 3; (eee) E, R at a residue corresponding to position 74 in SEQ ID NO: 3; (fff) G at a residue corresponding to position 75 in SEQ ID NO: 3; (ggg) Y at a residue corresponding to position 77 in SEQ ID NO: 3; (hhh) E, F, Y, H, N at a residue corresponding to position 78 in SEQ ID NO: 3; (iii) D, W at a residue corresponding to position 79 in SEQ ID NO: 3; (jjj) F, V, I at a residue corresponding to position 80 in SEQ ID NO: 3; (kkk) A, R at a residue corresponding to position 81 in SEQ ID NO: 3; (lll) G at a residue corresponding to position 82 in SEQ ID NO: 3; (mmm) E at a residue corresponding to position 83 in SEQ ID NO: 3; (nnn) R, Q at a residue corresponding to position 84 in SEQ ID NO: 3; (ooo) V, K at a residue corresponding to position 86 in SEQ ID NO: 3; (ppp) M at a residue corresponding to position 88 in SEQ ID NO: 3; (qqq) V, L at a residue corresponding to position 89 in SEQ ID NO: 3; (rrr) S, D, H, E, R at a residue corresponding to position 90 in SEQ ID NO: 3; (sss) E, Q at a residue corresponding to position 91 in SEQ ID NO: 3; (ttt) D at a residue corresponding to position 92 in SEQ ID NO: 3; (uuu) D at a residue corresponding to position 93 in SEQ ID NO: 3; (vvv) G, D at a residue corresponding to position 94 in SEQ ID NO: 3; (www) K, M, R at a residue corresponding to position 95 in SEQ ID NO: 3; (xxx) N at a residue corresponding to position 96 in SEQ ID NO: 3; (yyy) I, V, M at a residue corresponding to position 97 in SEQ ID NO: 3; (zzz) Y, I, M, T at a residue corresponding to position 98 in SEQ ID NO: 3; (aaaa) R, T, Q, K at a residue corresponding to position 99 in SEQ ID NO: 3; (bbbb) Y, I, M, L at a residue corresponding to position 100 in SEQ ID NO: 3; (cccc) Y, H at a residue corresponding to position 101 in SEQ ID NO: 3; (dddd) R, Q at a residue corresponding to position 102 in SEQ ID NO: 3; (eeee) F, M at a residue corresponding to position 104 in SEQ ID NO: 3; (ffff) C, V at a residue corresponding to position 107 in SEQ ID NO: 3; (gggg) T, S at a residue corresponding to position 111 in SEQ ID NO: 3; (hhhh) I, T, L at a residue corresponding to position 117 in SEQ ID NO: 3; (iiii) T, S at a residue corresponding to position 119 in SEQ ID NO: 3; (jjjj) A, T, M at a residue corresponding to position 120 in SEQ ID NO: 3; (kkkk) L, V, I at a residue corresponding to position 121 in SEQ ID NO: 3; (llll) H at a residue corresponding to position 122 in SEQ ID NO: 3; (mmmm) I, V, L, M at a residue corresponding to position 124 in SEQ ID NO: 3; (nnnn) I, L, V at a residue corresponding to position 125 in SEQ ID NO: 3; (oooo) A at a residue corresponding to position 126 in SEQ ID NO: 3; (pppp) I at a residue corresponding to position 128 in SEQ ID NO: 3; (qqqq) A, M at a residue corresponding to position 129 in SEQ ID NO: 3; (rrrr) A, S at a residue corresponding to position 130 in SEQ ID NO: 3; (ssss) C, T at a residue corresponding to position 133 in SEQ ID NO: 3; (tttt) L, I at a residue corresponding to position 136 in SEQ ID NO: 3; (uuuu) R at a residue corresponding to position 144 in SEQ ID NO: 3; (vvvv) C, T, V at a residue corresponding to position 145 in SEQ ID NO: 3; (wwww) N at a residue corresponding to position 146 in SEQ ID NO: 3; (xxxx) A, I, M at a residue corresponding to position 147 in SEQ ID NO: 3; (yyyy) E, L, A, R at a residue corresponding to position 148 in SEQ ID NO: 3; (zzzz) M, V, L, I at a residue corresponding to position 149 in SEQ ID NO: 3; (aaaaa) A, I, M at a residue corresponding to position 150 in SEQ ID NO: 3; (bbbbb) K, D, Q at a residue corresponding to position 151 in SEQ ID NO: 3; (ccccc) M, I at a residue corresponding to position 152 in SEQ ID NO: 3; (ddddd) M, C, F, V at a residue corresponding to position 153 in SEQ ID NO: 3; (eeeee) Q, K at a residue corresponding to position 154 in SEQ ID NO: 3; (fffff) K, R, D, Q at a residue corresponding to position 155 in SEQ ID NO: 3; (ggggg) I at a residue corresponding to position 156 in SEQ ID NO: 3; (hhhhh) A, F, I at a residue corresponding to position 157 in SEQ ID NO: 3; (iiiii) E, V, K at a residue corresponding to position 158 in SEQ ID NO: 3; (jjjjj) D, K at a residue corresponding to position 159 in SEQ ID NO: 3; (kkkkk) V, A at a residue corresponding to position 160 in SEQ ID NO: 3; (lllll) F, Y, V, A, L at a residue corresponding to position 162 in SEQ ID NO: 3; (mmmmm) A, D, P at a residue corresponding to position 164 in SEQ ID NO: 3; (nnnnn) D, H at a residue corresponding to position 165 in SEQ ID NO: 3; (ooooo) V, C, I at a residue corresponding to position 166 in SEQ ID NO: 3; (ppppp) L, V, F at a residue corresponding to position 167 in SEQ ID NO: 3; (qqqqq) T, N at a residue corresponding to position 168 in SEQ ID NO: 3; (rrrrr) C, L, M, I at a residue corresponding to position 169 in SEQ ID NO: 3; (sssss) V at a residue corresponding to position 170 in SEQ ID NO: 3; (ttttt) D, E, P, N at a residue corresponding to position 171 in SEQ ID NO: 3; (uuuuu) D, G, H at a residue corresponding to position 172 in SEQ ID NO: 3; (vvvvv) T at a residue corresponding to position 174 in SEQ ID NO: 3; (wwwww) I, H at a residue corresponding to position 175 in SEQ ID NO: 3; (xxxxx) D at a residue corresponding to position 176 in SEQ ID NO: 3; (yyyyy) A, T, I at a residue corresponding to position 177 in SEQ ID NO: 3; (zzzzz) E at a residue corresponding to position 180 in SEQ ID NO: 3; (aaaaaa) I at a residue corresponding to position 181 in SEQ ID NO: 3; (bbbbbb) Q, A, H, K at a residue corresponding to position 183 in SEQ ID NO: 3; (cccccc) G, S at a residue corresponding to position 184 in SEQ ID NO: 3; (dddddd) M, V, I, F, K at a residue corresponding to position 187 in SEQ ID NO: 3; (eeeeee) V, L at a residue corresponding to position 189 in SEQ ID NO: 3; (ffffff) A at a residue corresponding to position 190 in SEQ ID NO: 3; (gggggg) A, T, R, K at a residue corresponding to position 194 in SEQ ID NO: 3; (hhhhhh) V at a residue corresponding to position 196 in SEQ ID NO: 3; (iiiiii) I at a residue corresponding to position 197 in SEQ ID NO: 3; (jjjjjj) R at a residue corresponding to position 198 in SEQ ID NO: 3; (kkkkkk) V at a residue corresponding to position 199 in SEQ ID NO: 3; (llllll) C at a residue corresponding to position 200 in SEQ ID NO: 3; (mmmmmm) A at a residue corresponding to position 201 in SEQ ID NO: 3; (nnnnnn) A, T, R at a residue corresponding to position 202 in SEQ ID NO: 3; (oooooo) N, H, A at a residue corresponding to position 203 in SEQ ID NO: 3; (pppppp) V at a residue corresponding to position 206 in SEQ ID NO: 3; (qqqqqq) V, T, C at a residue corresponding to position 207 in SEQ ID NO: 3; (rrrrrr) V, M, Y at a residue corresponding to position 208 in SEQ ID NO: 3; (ssssss) A, P at a residue corresponding to position 212 in SEQ ID NO: 3; (tttttt) S at a residue corresponding to position 214 in SEQ ID NO: 3; (uuuuuu) P, N, C, A, S at a residue corresponding to position 215 in SEQ ID NO: 3; (vvvvvv) A, P at a residue corresponding to position 216 in SEQ ID NO: 3; (wwwwww) M, A, C at a residue corresponding to position 217 in SEQ ID NO: 3; (xxxxxx) I, V at a residue corresponding to position 218 in SEQ ID NO: 3; (yyyyyy) I, M, L at a residue corresponding to position 219 in SEQ ID NO: 3; (zzzzzz) P, D at a residue corresponding to position 221 in SEQ ID NO: 3; (aaaaaaa) T, S at a residue corresponding to position 222 in SEQ ID NO: 3; (bbbbbbb) C at a residue corresponding to position 223 in SEQ ID NO: 3; (ccccccc) N at a residue corresponding to position 224 in SEQ ID NO: 3; (ddddddd) V at a residue corresponding to position 225 in SEQ ID NO: 3; (eeeeeee) M, E, K at a residue corresponding to position 227 in SEQ ID NO: 3; (fffffff) A at a residue corresponding to position 228 in SEQ ID NO: 3; (ggggggg) V at a residue corresponding to position 229 in SEQ ID NO: 3; (hhhhhhh) D, E, P, K at a residue corresponding to position 230 in SEQ ID NO: 3; (iiiiiii) R, Q, I, K at a residue corresponding to position 231 in SEQ ID NO: 3; (jjjjjjj) M at a residue corresponding to position 232 in SEQ ID NO: 3; (kkkkkkk) L at a residue corresponding to position 233 in SEQ ID NO: 3; (lllllll) F, A, W, M, Q at a residue corresponding to position 234 in SEQ ID NO: 3; (mmmmmmm) F, Y, C, M at a residue corresponding to position 237 in SEQ ID NO: 3; (nnnnnnn) R, Q, M at a residue corresponding to position 238 in SEQ ID NO: 3; (ooooooo) T at a residue corresponding to position 240 in SEQ ID NO: 3; (ppppppp) V at a residue corresponding to position 243 in SEQ ID NO: 3; (qqqqqqq) I at a residue corresponding to position 245 in SEQ ID NO: 3; (rrrrrrr) S, A at a residue corresponding to position 246 in SEQ ID NO: 3; (sssssss) N at a residue corresponding to position 248 in SEQ ID NO: 3; (ttttttt) Y at a residue corresponding to position 249 in SEQ ID NO: 3; (uuuuuuu) M at a residue corresponding to position 250 in SEQ ID NO: 3; (vvvvvvv) F, L at a residue corresponding to position 251 in SEQ ID NO: 3; (wwwwwww) V, I at a residue corresponding to position 252 in SEQ ID NO: 3; (xxxxxxx) H at a residue corresponding to position 253 in SEQ ID NO: 3; (yyyyyyy) K, R, D at a residue corresponding to position 254 in SEQ ID NO: 3; (zzzzzzz) E at a residue corresponding to position 255 in SEQ ID NO: 3; (aaaaaaaa) I, M, V, R at a residue corresponding to position 256 in SEQ ID NO: 3; (bbbbbbbb) Y, A at a residue corresponding to position 257 in SEQ ID NO: 3; (cccccccc) D at a residue corresponding to position 258 in SEQ ID NO: 3; (dddddddd) R, A, Q, D at a residue corresponding to position 259 in SEQ ID NO: 3; (eeeeeeee) V, L at a residue corresponding to position 260 in SEQ ID NO: 3; (ffffffff) V, L, M at a residue corresponding to position 261 in SEQ ID NO: 3; (gggggggg) K at a residue corresponding to position 262 in SEQ ID NO: 3; (hhhhhhhh) K, R at a residue corresponding to position 263 in SEQ ID NO: 3; (iiiiiiii) V, F at a residue corresponding to position 264 in SEQ ID NO: 3; (jjjjjjjj) R, Q, E at a residue corresponding to position 265 in SEQ ID NO: 3; (kkkkkkkk) E at a residue corresponding to position 266 in SEQ ID NO: 3; (llllllll) R at a residue corresponding to position 267 in SEQ ID NO: 3; (mmmmmmmm) F, L, W, M, I at a residue corresponding to position 270 in SEQ ID NO: 3; (nnnnnnnn) W, F, K, H at a residue corresponding to position 271 in SEQ ID NO: 3; (oooooooo) V, L, M at a residue corresponding to position 272 in SEQ ID NO: 3; (pppppppp) C at a residue corresponding to position 273 in SEQ ID NO: 3; (qqqqqqqq) D, E at a residue corresponding to position 274 in SEQ ID NO: 3; (rrrrrrrr) A, P, G, E at a residue corresponding to position 275 in SEQ ID NO: 3; (ssssssss) R, D at a residue corresponding to position 276 in SEQ ID NO: 3; (tttttttt) E, Q at a residue corresponding to position 277 in SEQ ID NO: 3; (uuuuuuuu) E at a residue corresponding to position 278 in SEQ ID NO: 3; (vvvvvvvv) S, E at a residue corresponding to position 279 in SEQ ID NO: 3; (wwwwwwww) T at a residue corresponding to position 280 in SEQ ID NO: 3; (xxxxxxxx) I, V, M at a residue corresponding to position 281 in SEQ ID NO: 3; (yyyyyyyy) K, Q, G, M at a residue corresponding to position 282 in SEQ ID NO: 3; (zzzzzzzz) E, N, H at a residue corresponding to position 283 in SEQ ID NO: 3; (aaaaaaaaa) V at a residue corresponding to position 284 in SEQ ID NO: 3; (bbbbbbbbb) V at a residue corresponding to position 285 in SEQ ID NO: 3; (ccccccccc) Y, V, I at a residue corresponding to position 286 in SEQ ID NO: 3; (ddddddddd) E at a residue corresponding to position 288 in SEQ ID NO: 3; (eeeeeeeee) H at a residue corresponding to position 290 in SEQ ID NO: 3; (fffffffff) L, A, T, H, N, K at a residue corresponding to position 291 in SEQ ID NO: 3; (ggggggggg) A, I, N at a residue corresponding to position 293 in SEQ ID NO: 3; (hhhhhhhhh) R at a residue corresponding to position 294 in SEQ ID NO: 3; (iiiiiiiii) D, R, Q at a residue corresponding to position 295 in SEQ ID NO: 3; (jjjjjjjjj) L, W at a residue corresponding to position 296 in SEQ ID NO: 3; (kkkkkkkkk) K, Q, R at a residue corresponding to position 299 in SEQ ID NO: 3; (lllllllll) A at a residue corresponding to position 300 in SEQ ID NO: 3; (mmmmmmmmm) A at a residue corresponding to position 301 in SEQ ID NO: 3; (nnnnnnnnn) A, E, Q at a residue corresponding to position 302 in SEQ ID NO: 3; (ooooooooo) E, R at a residue corresponding to position 303 in SEQ ID NO: 3; (ppppppppp) L at a residue corresponding to position 304 in SEQ ID NO: 3; (qqqqqqqqq) V at a residue corresponding to position 305 in SEQ ID NO: 3; (rrrrrrrrr) E, H, K, R at a residue corresponding to position 306 in SEQ ID NO: 3; (sssssssss) I, M, V at a residue corresponding to position 307 in SEQ ID NO: 3; (ttttttttt) T at a residue corresponding to position 308 in SEQ ID NO: 3; (uuuuuuuuu) F, V, L at a residue corresponding to position 310 in SEQ ID NO: 3; (vvvvvvvvv) D, S, R, E at a residue corresponding to position 311 in SEQ ID NO: 3; (wwwwwwwww) A, T, D, K at a residue corresponding to position 312 in SEQ ID NO: 3; (xxxxxxxxx) V, L at a residue corresponding to position 313 in SEQ ID NO: 3; (yyyyyyyyy) D at a residue corresponding to position 315 in SEQ ID NO: 3; (zzzzzzzzz) G, E, N at a residue corresponding to position 316 in SEQ ID NO: 3; (aaaaaaaaaa) G, T at a residue corresponding to position 317 in SEQ ID NO: 3; (bbbbbbbbbb) R at a residue corresponding to position 318 in SEQ ID NO: 3; (cccccccccc) L, M at a residue corresponding to position 319 in SEQ ID NO: 3; (dddddddddd) Y, L at a residue corresponding to position 320 in SEQ ID NO: 3; (eeeeeeeeee) I, L, C at a residue corresponding to position 321 in SEQ ID NO: 3; (ffffffffff) G, V, A, T at a residue corresponding to position 322 in SEQ ID NO: 3; (gggggggggg) I, V, K, P at a residue corresponding to position 323 in SEQ ID NO: 3; (hhhhhhhhhh) I, V, L at a residue corresponding to position 324 in SEQ ID NO: 3; (iiiiiiiiii) E, T at a residue corresponding to position 325 in SEQ ID NO: 3; (jjjjjjjjjj) D at a residue corresponding to position 326 in SEQ ID NO: 3; (kkkkkkkkkk) F, W, V at a residue corresponding to position 327 in SEQ ID NO: 3; (llllllllll) S at a residue corresponding to position 328 in SEQ ID NO: 3; (mmmmmmmmmm) P, R, D, N at a residue corresponding to position 329 in SEQ ID NO: 3; (nnnnnnnnnn) E at a residue corresponding to position 330 in SEQ ID NO: 3; (oooooooooo) D at a residue corresponding to position 331 in SEQ ID NO: 3; (pppppppppp) T, R at a residue corresponding to position 332 in SEQ ID NO: 3; (qqqqqqqqqq) I, M, F at a residue corresponding to position 333 in SEQ ID NO: 3; (rrrrrrrrrr) S, V, C, M at a residue corresponding to position 334 in SEQ ID NO: 3; (ssssssssss) R at a residue corresponding to position 335 in SEQ ID NO: 3; (tttttttttt) M at a residue corresponding to position 339 in SEQ ID NO: 3; (uuuuuuuuuu) I at a residue corresponding to position 341 in SEQ ID NO: 3; (vvvvvvvvvv) I, M at a residue corresponding to position 342 in SEQ ID NO: 3; (wwwwwwwwww) S, C, V at a residue corresponding to position 343 in SEQ ID NO: 3; (xxxxxxxxxx) M, I, V, L at a residue corresponding to position 344 in SEQ ID NO: 3; (yyyyyyyyyy) L, M, Y, V at a residue corresponding to position 345 in SEQ ID NO: 3; (zzzzzzzzzz) P at a residue corresponding to position 346 in SEQ ID NO: 3; (aaaaaaaaaaa) F, V at a residue corresponding to position 347 in SEQ ID NO: 3; (bbbbbbbbbbb) E, K, S at a residue corresponding to position 348 in SEQ ID NO: 3; (ccccccccccc) G, T, N, S, D at a residue corresponding to position 349 in SEQ ID NO: 3; (ddddddddddd) E, I, V, F, L at a residue corresponding to position 350 in SEQ ID NO: 3; (eeeeeeeeeee) D at a residue corresponding to position 351 in SEQ ID NO: 3; (fffffffffff) L, C at a residue corresponding to position 354 in SEQ ID NO: 3; (ggggggggggg) D, A, Q, S, E at a residue corresponding to position 355 in SEQ ID NO: 3; (hhhhhhhhhhh) L, V at a residue corresponding to position 356 in SEQ ID NO: 3; (iiiiiiiiii) R, N at a residue corresponding to position 358 in SEQ ID NO: 3; (jjjjjjjjjjj) S, A, N, E at a residue corresponding to position 359 in SEQ ID NO: 3; (kkkkkkkkkkk) S at a residue corresponding to position 360 in SEQ ID NO: 3; (lllllllllll) S, N, T at a residue corresponding to position 362 in SEQ ID NO: 3; (mmmmmmmmmmm) E at a residue corresponding to position 363 in SEQ ID NO: 3; (nnnnnnnnnnn) A, K, Y, L at a residue corresponding to position 366 in SEQ ID NO: 3; (ooooooooooo) C, A at a residue corresponding to position 370 in SEQ ID NO: 3; (ppppppppppp) S, A at a residue corresponding to position 371 in SEQ ID NO: 3; (qqqqqqqqqqq) V, M at a residue corresponding to position 372 in SEQ ID NO: 3; (rrrrrrrrrrr) F, Y, W at a residue corresponding to position 373 in SEQ ID NO: 3; (sssssssssss) T at a residue corresponding to position 374 in SEQ ID NO: 3; (ttttttttttt) H, T, K, R, Q at a residue corresponding to position 375 in SEQ ID NO: 3; (uuuuuuuuuuu) D at a residue corresponding to position 376 in SEQ ID NO: 3; (vvvvvvvvvvv) E, I, V at a residue corresponding to position 377 in SEQ ID NO: 3; (wwwwwwwwwww) K, A, D at a residue corresponding to position 378 in SEQ ID NO: 3; (xxxxxxxxxxx) R, N, V at a residue corresponding to position 379 in SEQ ID NO: 3; (yyyyyyyyyyy) V at a residue corresponding to position 380 in SEQ ID NO: 3; (zzzzzzzzzzz) W, H, M, R, K, T at a residue corresponding to position 381 in SEQ ID NO: 3; (aaaaaaaaaaaa) K at a residue corresponding to position 382 in SEQ ID NO: 3; (bbbbbbbbbbbb) I, M at a residue corresponding to position 383 in SEQ ID NO: 3; (cccccccccccc) G, S at a residue corresponding to position 384 in SEQ ID NO: 3; (dddddddddddd) R, K at a residue corresponding to position 385 in SEQ ID NO: 3; (eeeeeeeeeeee) Q, K, R at a residue corresponding to position 386 in SEQ ID NO: 3; (ffffffffffff) M, V, L at a residue corresponding to position 387 in SEQ ID NO: 3; (gggggggggggg) R, E, Q, H, D at a residue corresponding to position 388 in SEQ ID NO: 3; (hhhhhhhhhhhh) T, A, C at a residue corresponding to position 389 in SEQ ID NO: 3; (iiiiiiiiiiii) G, T, C at a residue corresponding to position 390 in SEQ ID NO: 3; (jjjjjjjjjjjj) M, V, I at a residue corresponding to position 392 in SEQ ID NO: 3; (kkkkkkkkkkkk) G at a residue corresponding to position 393 in SEQ ID NO: 3; (llllllllllll) I at a residue corresponding to position 394 in SEQ ID NO: 3; (mmmmmmmmmmmm) E, D, S at a residue corresponding to position 396 in SEQ ID NO: 3; (nnnnnnnnnnnn) W, I, Y at a residue corresponding to position 397 in SEQ ID NO: 3; (oooooooooooo) S, I, H at a residue corresponding to position 400 in SEQ ID NO: 3; (pppppppppppp) D, G, S, H, A at a residue corresponding to position 401 in SEQ ID NO: 3; (qqqqqqqqqqqq) M at a residue corresponding to position 402 in SEQ ID NO: 3; (rrrrrrrrrrrr) V, G at a residue corresponding to position 405 in SEQ ID NO: 3; (ssssssssssss) W, F, L at a residue corresponding to position 406 in SEQ ID NO: 3; (tttttttttttt) H, Q at a residue corresponding to position 407 in SEQ ID NO: 3; (uuuuuuuuuuuu) M at a residue corresponding to position 410 in SEQ ID NO: 3; (vvvvvvvvvvvv) G, A, P, T at a residue corresponding to position 411 in SEQ ID NO: 3; (wwwwwwwwwwww) V at a residue corresponding to position 414 in SEQ ID NO: 3; (xxxxxxxxxxxx) S at a residue corresponding to position 420 in SEQ ID NO: 3; (yyyyyyyyyyyy) F at a residue corresponding to position 421 in SEQ ID NO: 3; (zzzzzzzzzzzz) G, R at a residue corresponding to position 423 in SEQ ID NO: 3; (aaaaaaaaaaaaa) I, A, G at a residue corresponding to position 425 in SEQ ID NO: 3; (bbbbbbbbbbbbb) V at a residue corresponding to position 426 in SEQ ID NO: 3; (ccccccccccccc) A, T at a residue corresponding to position 427 in SEQ ID NO: 3; (ddddddddddddd) D at a residue corresponding to position 428 in SEQ ID NO: 3; (eeeeeeeeeeeee) N at a residue corresponding to position 431 in SEQ ID NO: 3; (fffffffffffff) T, H, K, M, R at a residue corresponding to position 433 in SEQ ID NO: 3; (ggggggggggggg) M, V at a residue corresponding to position 435 in SEQ ID NO: 3; (hhhhhhhhhhhhh) V, F, I at a residue corresponding to position 436 in SEQ ID NO: 3; (iiiiiiiiiiiii) Q at a residue corresponding to position 437 in SEQ ID NO: 3; (jjjjjjjjjjjjj) I, T, Y, C at a residue corresponding to position 438 in SEQ ID NO: 3; (kkkkkkkkkkkkk) S at a residue corresponding to position 439 in SEQ ID NO: 3; (lllllllllllll) W, K at a residue corresponding to position 440 in SEQ ID NO: 3; (mmmmmmmmmmmmm) M, I, L at a residue corresponding to position 441 in SEQ ID NO: 3; (nnnnnnnnnnnnn) W, F, R at a residue corresponding to position 443 in SEQ ID NO: 3; (ooooooooooooo) K at a residue corresponding to position 444 in SEQ ID NO: 3; (ppppppppppppp) K at a residue corresponding to position 446 in SEQ ID NO: 3; (qqqqqqqqqqqqq) E, N at a residue corresponding to position 447 in SEQ ID NO: 3; (rrrrrrrrrrrrr) W, K, P at a residue corresponding to position 448 in SEQ ID NO: 3; (sssssssssssss) R, H at a residue corresponding to position 449 in SEQ ID NO: 3; (ttttttttttttt) V at a residue corresponding to position 450 in SEQ ID NO: 3; (uuuuuuuuuuuuu) S, K at a residue corresponding to position 452 in SEQ ID NO: 3; (vvvvvvvvvvvvv) H, P at a residue corresponding to position 453 in SEQ ID NO: 3; (wwwwwwwwwwwww) A, D at a residue corresponding to position 454 in SEQ ID NO: 3; (xxxxxxxxxxxxx) K, D at a residue corresponding to position 455 in SEQ ID NO: 3; (yyyyyyyyyyyyy) F at a residue corresponding to position 457 in SEQ ID NO: 3; (zzzzzzzzzzzzz) H, G at a residue corresponding to position 458 in SEQ ID NO: 3; (aaaaaaaaaaaaaa) D at a residue corresponding to position 459 in SEQ ID NO: 3; or (bbbbbbbbbbbbbb) any combination thereof.

In some embodiments, an engineered ALDH described herein having one or more amino acid alterations results in an engineered ALDH comprising: (a) V at a residue corresponding to position 42 in SEQ ID NO: 3; (b) T or S at a residue corresponding to position 119 in SEQ ID NO: 3; (c) I at a residue corresponding to position 149 in SEQ ID NO: 3; (d) K at a residue corresponding to position 158 in SEQ ID NO: 3; (e) T at a residue corresponding to position 174 in SEQ ID NO: 3; (f) A at a residue corresponding to position 246 in SEQ ID NO: 3; (g) D at a residue corresponding to position 259 in SEQ ID NO: 3; (h) R at a residue corresponding to position 263 in SEQ ID NO: 3; (i) R at a residue corresponding to position 299 in SEQ ID NO: 3; (j) V at a residue corresponding to position 327 in SEQ ID NO: 3; (k) S at a residue corresponding to position 348 in SEQ ID NO: 3; (l) A at a residue corresponding to position 370 in SEQ ID NO: 3; (m) I at a residue corresponding to position 392 in SEQ ID NO: 3; (n) A at a residue corresponding to position 401 in SEQ ID NO: 3; (o) T at a residue corresponding to position 427 in SEQ ID NO: 3; or (p) any combination thereof.

In some embodiments, an engineered ALDH described herein comprises one or more amino acid alterations selected from the group consisting of: E2P, E2Q, E2T, Q3E, Q3K, Q3M, Q3R, Q3S, A4G, A4S, V5I, V5T, K6E, K6F, K6H, K6P, K6Q, K6R, D7E, D7Q, Y8D, L9I, L9V, D10N, K11A, K11D, K11E, K11Q, K11R, M12A, M12I, M12L, V13L, A14D, A14E, A14K, A14Q, A15K, A15R, S16A, R17H, R17K, R17Q, 118E, I18K, I18Q, I18T, Q20F, Q20H, Q20M, Q21A, Q21E, Q21K, Q21P, Q21R, E22A, E22K, E22Q, F23L, F23W, F23Y, A24E, A24K, A24R, A24S, T25A, T25E, T25K, T25N, T25S, Y26F, Y26L, Y26M, Y26T, Y26W, P27D, P27S, P27T, Q28W, E29K, E29Q, T30D, T30E, T30K, T30Q, T30R, V31I, V31L, V31R, D32A, D32E, D32N, K33A, K33E, K33Q, K33R, A34I, A34L, A34M, A34V, V35C, V35I, V35L, V35M, R36E, R36H, R36K, T37A, T37K, T37R, V38F, V38I, V38L, V38M, V38W, G39A, G39R, K40D, K40E, K40Q, K40R, K40W, A41L, A41V, I42C, I42L, I42M, I42V, Y43E, Y43L, Y43R, D44E, D44K, D44Q, D44R, N45H, N45Q, N45R, E47D, L48D, L48E, L48M, L48R, L49F, L49I, L49M, L49W, L49Y, A50G, H51A, H51E, H51K, H51N, H51Q, H51R, M52L, A53E, A53I, A53S, V54I, V54L, V54M, V54T, D55E, D55Q, D55R, K58E, K58G, K58Q, K58R, M59F, M59I, M59L, M59Y, G60A, N61D, N61E, N61K, N61R, Y62A, Y62F, Y62I, Y62M, Y62V, Y62W, A63D, A63E, A63Q, D64E, D64H, K65E, K65R, I66L, I66V, A67E, K68D, K68E, C69H, C69I, C69L, C69M, C69N, V70I, V70L, V70M, V70R, N71D, N71E, N71F, N71H, N71Y, K72A, K72R, S73A, S73T, K74D, K74E, K74R, S75G, S75P, S75Y, V76F, V76I, V76L, V76M, V76Y, W77F, W77Y, W78E, W78F, W78H, W78N, W78R, W78Y, R79D, R79S, R79W, R79Y, M80F, M80I, M80L, M80V, K81A, K81E, K81R, D82E, D82G, D82P, K83E, K83Q, K84Q, K84R, S85A, S85T, R86E, R86K, R86V, G87T, I88L, I88M, I88V, I89L, I89P, I89V, K90D, K90E, K90H, K90P, K90R, K90S, R91D, R91E, R91Q, I92D, I92M, P93D, P93E, E94A, E94D, E94G, E94N, L95K, L95M, L95N, L95R, G96H, G96K, G96N, G96R, L97I, L97M, L97V, V98I, V98M, V98T, V98Y, E99K, E99Q, E99R, E99T, V100Y, V100L, V100M, V100I, A101K, A101Y, A101R, A101H, K102E, K102Q, K102R, K102V, I104V, I104Y, I104F, I104L, I104M, V106I, I107C, I107V, G108A, G108C, C109I, C109A, C109M, V110M, V110L, V110I, A111T, A111V, A111Y, A111I, A111S, P112E, T113F, T113S, T113W, T113Y, T114N, N115W, N115R, N115H, N115Y, V117N, V117L, V117T, V117I, I118N, I118A, I118L, I118M, I118S, I118F, I118V, N119Q, N119I, N119L, N119M, N119T, N119S, V120T, V120A, V120P, V120M, M121V, M121A, M121L, M121C, M121I, Q122C, Q122F, Q122R, Q122Y, Q122H, Q122N, Q122S, Q122G, Q122V, Q122W, N123K, N123P, A124V, A124L, A124M, A124I, M125I, M125L, M125A, M125V, C126A, C126P, A127M, L128I, K129A, K129M, C130T, C130S, C130A, G131R, N132C, S133T, S133C, M134I, M134C, M134V, I135V, I135M, V136L, V136M, V136F, V136I, S137R, S137K, P138G, H139S, H139G, P140E, R141Q, R141K, R141Y, R141E, A142T, A142S, K143P, H144L, H144N, H144R, S145T, S145C, S145V, S146T, S146C, S146A, S146N, V147I, V147M, V147A, K148Y, K148L, K148A, K148E, K148H, K148R, T149V, T149I, T149L, T149M, T149F, V150I, V150M, V150G, V150A, E151D, E151K, E151R, E151Q, V152L, V152M, V152I, I153F, I153V, I153C, I153M, N154H, N154A, N154Q, N154K, N154R, E155R, E155K, E155D, E155Q, A156I, L157A, L157I, L157F, A158E, A158V, A158K, A159K, A159Q, A159D, A159E, L160V, L160C, L160A, M162I, M162A, M162F, M162Y, M162L, M162V, K164D, K164A, K164P, N165H, N165G, N165D, L166C, L166A, L166I, L166V, I167V, I167F, I167L, Q168T, Q168N, V169L, V169W, V169C, V169M, V169I, I170L, I170V, T171E, T171P, T171N, T171D, E172D, E172G, E172H, P173G, P173D, S174G, S174D, S174T, M175I, M175H, M175R, E176Q, E176D, L177V, L177T, L177A, L177I, S178V, S178A, S178T, S178G, A179D, A179N, A179E, G180H, G180Q, G180A, G180E, L181I, L181F, M182L, M182H, M182I, M182V, S183Q, S183K, S183D, S183A, S183H, S183E, A184H, A184P, A184S, A184G, V185C, V185I, D186R, L187H, L187K, L187F, L187I, L187V, L187M, C188I, C188L, C188V, I189S, I189F, I189T, I189M, I189V, I189L, C190P, C190A, C190F, T191R, G193S, P194T, P194R, P194K, P194A, G195A, L196M, L196I, L196V, V197I, V197M, V197G, V197A, K198Q, K198E, K198R, A199S, A199V, A200C, A200I, A200V, Y201M, Y201Q, Y201A, S202N, S202K, S202A, S202R, S202T, S202E, S202Q, S203N, S203H, S203A, K205R, K205T, P206V, P206R, P206K, A207V, A207S, A207C, A207T, I208V, I208T, I208Y, I208M, I208L, G209L, G209E, G209M, V210H, V210L, G211L, Q212A, Q212P, Q212G, G213K, N214S, N214K, V215S, V215N, V215A, V215C, V215D, V215P, Q216H, Q216A, Q216P, V217F, V217A, V217I, V217C, V217M, L218I, L218V, L218F, L218Y, V219M, V219F, V219L, V219I, D220C, D220F, R221D, R221P, D222T, D222S, A223C, D224N, L225V, D226E, Q227K, Q227R, Q227M, Q227E, V228A, A229W, A229V, A230R, A230D, A230P, A230Q, A230K, A230E, M231Q, M231R, M231I, M231K, V232I, V232F, V232T, V232C, V232Q, V232M, V232N, I233V, I233L, I233M, K234W, K234Q, K234F, K234A, K234M, G235A, G235S, R236K, R236C, R236W, R236A, R236I, T237F, T237Y, T237C, T237M, F238R, F238M, F238Q, F238Y, F238W, D239R, D239H, D239Q, N240Q, N240A, N240T, N240C, N240S, G241N, L243V, L243M, L243T, L243C, T245M, T245I, T245N, T245S, T245V, T245A, T245C, C246S, C246A, E247G, E247P, E247I, E247A, Q248D, Q248T, Q248E, Q248K, Q248S, Q248N, N249Y, N249R, V250I, V250L, V250M, I251Y, 1251F, 1251L, I251V, C252V, C252I, P253D, P253Q, P253H, P253V, P253E, E254Q, E254K, E254R, E254D, D255E, K256V, K256M, K256I, K256R, K257A, K257F, K257H, K257Y, E258D, E259A, E259R, E259K, E259D, E259Q, M260I, M260V, M260F, M260L, I261L, I261M, I261V, A262Q, A262D, A262E, A262K, A263E, A263K, A263R, L264M, L264V, L264F, K265Q, K265R, K265A, K265E, K266A, K266E, H267Y, H267R, H267N, H267A, H267Q, G268A, A269S, A269G, A269C, Y270L, Y270F, Y270I, Y270M, Y270W, Y271F, Y271K, Y271V, Y271W, Y271R, Y271H, I272V, I272M, I272L, G273C, N274E, N274D, S275P, S275G, S275A, S275E, E276Q, E276M, E276R, E276D, D277Q, D277E, A278P, A278E, A279E, A279D, A279S, K280Q, K280R, K280T, L281V, L281M, L281I, R282Q, R282K, R282M, R282G, R282E, D283E, D283H, D283N, T284V, T284P, A285L, A285V, A285I, A285M, F286L, F286I, F286W, F286Y, F286V, N288R, N288D, N288E, G289M, G290H, P291K, P291L, P291E, P291T, P291H, P291A, P291N, V292I, V292M, S293A, S293V, S293I, S293N, K294S, K294N, K294P, K294E, K294R, E295R, E295K, E295A, E295Q, E295D, Y296W, Y296R, Y296V, Y296F, Y296H, Y296L, P297A, P297T, P297Q, P297V, P297L, P297H, P297I, G298A, A299K, A299Q, A299R, S300A, S300R, V301I, V301A, K302A, K302R, K302D, K302Q, K302E, K303Q, K303W, K303R, K303E, I304L, I304G, I304Y, A305I, A305V, Q306H, Q306K, Q306E, Q306R, L307I, L307V, L307M, S308A, S308T, I310G, I310V, I310L, I310F, Q311R, Q311T, Q311K, Q311S, Q311D, Q311E, G312A, G312D, G312T, G312K, I313L, I313V, P314A, E315D, D316E, D316G, D316N, A317G, A317T, K318R, K318T, K318Y, V319F, V319L, V319M, V319I, V319Y, I320Y, I320L, V321L, V321P, V321C, V321I, S322A, S322T, S322G, S322V, C323E, C323L, C323I, C323G, C323P, C323V, C323K, T324I, T324V, T324L, R325K, R325E, R325D, R325T, G326D, Y327W, Y327F, Y327V, G328D, G328T, G328S, K329E, K329R, K329P, K329D, K329N, D330E, E331S, E331D, E331H, E331M, E331Y, P332R, P332T, L333M, L333V, L333F, L333I, A334V, A334S, A334M, A334C, K335R, K335Q, K335H, K337Q, K337E, L338F, L338V, L338I, L338M, F339C, F339M, V341I, L342M, L342V, L342I, A343C, A343T, A343V, A343S, A343P, F344I, F344V, F344L, F344M, F345Y, F345V, F345M, F345L, F345I, T346R, T346K, T346P, Y347F, Y347V, D348E, D348K, D348S, K349S, K349G, K349D, K349N, K349T, W350V, W350E, W350I, W350L, W350F, E351D, D352Q, D352H, D352E, I354L, I354V, I354C, H355R, H355A, H355D, H355Q, H355S, H355E, I356M, I356L, I356V, A357C, K358N, K358R, T359A, T359D, T359S, T359N, T359E, N360S, N360T, L361D, L361V, E362L, E362T, E362N, E362S, M363Y, M363E, E364Q, E364Y, E364F, E364H, I366Y, I366K, I366F, I366A, I366L, H368L, H368A, S369T, S369A, V370C, V370A, V371M, V371I, V371A, V371W, V371S, V371Y, I372V, I372M, H373W, H373Y, H373F, S374T, N375H, N375Q, N375K, N375T, N375R, T376N, T376S, T376D, P377D, P377I, P377V, P377E, E378K, E378D, E378A, H379N, H379K, H379V, H379R, I380A, I380M, I380V, E381M, E381W, E381K, E381R, E381T, E381H, A382K, A382R, A382E, A382Q, V383M, V383L, V383Y, V383F, V383I, A384S, A384G, E385Q, E385L, E385R, E385K, A386E, A386Q, A386K, A386R, I387M, I387V, I387L, P388R, P388H, P388D, P388Q, P388E, V389A, V389C, V389T, S390T, S390N, S390C, S390G, R391M, R391T, R391I, F392V, F392I, F392M, A393M, A393I, A393W, A393L, A393G, V394I, V394K, Q396R, Q396E, Q396S, Q396D, V397I, V397W, V397A, V397P, V397F, V397Y, G398S, G398C, G398P, G398T, G399A, G399S, T400I, T400S, T400F, T400V, T400H, N401G, N401H, N401D, N401S, N401A, L402N, L402A, L402F, L402M, G403S, A405I, A405F, A405V, A405G, M406Y, M406K, M406F, M406L, M406W, D407H, D407Q, D407Y, D407E, N408T, N408S, N408H, L410F, L410I, L410M, N411T, N411G, N411P, N411A, P412F, P412R, T413A, T414M, T414F, T414V, T415P, T415S, L416M, L416F, L416I, C418F, C418I, C418T, T420S, T420R, W421Y, W421F, N423K, N423R, N423G, N424S, N424R, S425I, S425G, S425W, S425A, I426V, I426T, I426L, S427G, S427T, S427A, E428D, E428H, N429E, F430I, F430Y, F430L, F430V, T431G, T431S, T431N, Y432A, Y432P, Y432F, Y432W, Y433T, Y433R, Y433K, Y433H, Y433M, Y433E, H434R, H434Q, H434D, H434N, L435V, L435F, L435W, L435Y, L435M, M436T, M436L, M436F, M436I, M436V, N437H, N437Q, N437Y, L438V, L438T, L438W, L438C, L438Y, L438I, T439Q, T439S, T439R, T439K, R440K, R440W, V441L, V441I, V441M, S442G, S442A, Y443H, Y443W, Y443R, Y443F, R444E, R444P, R444K, V445I, V445Y, V445F, V445M, V445L, P446E, P446K, D447E, D447N, M448W, M448K, M448P, Y449E, Y449R, Y449F, Y449H, I450M, I450C, I450F, I450V, I450L, P451W, T452S, T452D, T452K, D453E, D453P, D453H, E454A, E454D, E455K, E455Q, E455D, I456F, I456L, I456V, I456W, I456M, W457M, W457Y, W457F, A458H, A458G, E459D, and any combination thereof.

In some embodiments, an engineered ALDH described herein comprises one or more amino acid alterations selected from the group consisting of: E2Q, E2T, E2P, Q3S, Q3K, A4G, A4S, V5I, V5T, K6F, D7E, D7Q, L9V, K11E, K11D, K11Q, K11R, M12L, M12A, A15R, A15K, S16A, R17K, I18K, 118E, I18Q, Q20F, Q20H, Q21A, Q21R, E22K, E22Q, E22A, F23Y, A24E, A24K, T25A, T25E, Y26L, Y26M, Y26W, P27D, P27T, Q28W, T30D, T30K, T30Q, V31I, V31L, D32N, D32E, K33E, K33Q, K33A, K33R, A34V, V35C, V35M, R36K, T37K, T37A, T37R, V38L, V38I, G39A, K40W, A41L, A41V, I42L, I42V, I42M, I42C, Y43E, Y43L, D44K, D44Q, N45H, N45R, E47D, L48E, L48M, L48D, L49I, H51N, H51E, H51Q, M52L, V54L, D55E, D55R, K58R, N61D, N61E, N61R, Y62F, Y62V, Y62W, A63D, A63E, A63Q, D64H, K65R, I66V, A67E, V70M, V70R, N71D, N71E, N71Y, N71F, N71H, S73T, K74E, K74R, S75G, W77Y, W78E, W78F, W78Y, W78H, W78N, R79D, R79W, M80F, M80V, M80I, K81A, K81R, D82G, K83E, K84R, K84Q, R86V, R86K, I88M, I89V, I89L, K90S, K90D, K90H, K90E, K90R, R91E, R91Q, I92D, P93D, E94G, E94D, L95K, L95M, L95R, G96N, L97I, L97V, L97M, V98Y, V98I, V98M, V98T, E99R, E99T, E99Q, E99K, V100Y, V100I, V100M, V100L, A101Y, A101H, K102R, K102Q, I104F, I104M, I107C, I107V, A111T, A111S, V117I, V117T, V117L, N119T, N119S, V120A, V120T, V120M, M121L, M121V, M121I, Q122H, A124I, A124V, A124L, A124M, M125I, M125L, M125V, C126A, L128I, K129A, K129M, C130A, C130S, S133C, S133T, V136L, V136I, H144R, S145C, S145T, S145V, S146N, V147A, V147I, V147M, K148E, K148L, K148A, K148R, T149M, T149V, T149L, T149I, V150A, V150I, V150M, E151K, E151D, E151Q, V152M, V152I, I153M, I153C, I153F, I153V, N154Q, N154K, E155K, E155R, E155D, E155Q, A156I, L157A, L157F, L157I, A158E, A158V, A158K, A159D, A159K, L160V, L160A, M162F, M162Y, M162V, M162A, M162L, K164A, K164D, K164P, N165D, N165H, L166V, L166C, L166I, I167L, I167V, I167F, Q168T, Q168N, V169C, V169L, V169M, V169I, I170V, T171D, T171E, T171P, T171N, E172D, E172G, E172H, S174T, M175I, M175H, E176D, L177A, L177T, L177I, G180E, L181I, S183Q, S183A, S183H, S183K, A184G, A184S, L187M, L187V, L187I, L187F, L187K, I189V, I189L, C190A, P194A, P194T, P194R, P194K, L196V, V197I, K198R, A199V, A200C, Y201A, S202A, S202T, S202R, S203N, S203H, S203A, P206V, A207V, A207T, A207C, I208V, I208M, I208Y, Q212A, Q212P, N214S, V215P, V215N, V215C, V215A, V215S, Q216A, Q216P, V217M, V217A, V217C, L218I, L218V, V219I, V219M, V219L, R221P, R221D, D222T, D222S, A223C, D224N, L225V, Q227M, Q227E, Q227K, V228A, A229V, A230D, A230E, A230P, A230K, M231R, M231Q, M231I, M231K, V232M, I233L, K234F, K234A, K234W, K234M, K234Q, T237F, T237Y, T237C, T237M, F238R, F238Q, F238M, N240T, L243V, T245I, C246S, C246A, Q248N, N249Y, V250M, I251F, I251L, C252V, C252I, P253H, E254K, E254R, E254D, D255E, K256I, K256M, K256V, K256R, K257Y, K257A, E258D, E259R, E259A, E259Q, E259D, M260V, M260L, I261V, I261L, I261M, A262K, A263K, A263R, L264V, L264F, K265R, K265Q, K265E, K266E, H267R, Y270F, Y270L, Y270W, Y270M, Y270I, Y271W, Y271F, Y271K, Y271H, I272V, I272L, I272M, G273C, N274D, N274E, S275A, S275P, S275G, S275E, E276R, E276D, D277E, D277Q, A278E, A279S, A279E, K280T, L281I, L281V, L281M, R282K, R282Q, R282G, R282M, D283E, D283N, D283H, T284V, A285V, F286Y, F286V, F286I, N288E, G290H, P291L, P291A, P291T, P291H, P291N, P291K, S293A, S293I, S293N, K294R, E295D, E295R, E295Q, Y296L, Y296W, A299K, A299Q, A299R, S300A, V301A, K302A, K302E, K302Q, K303E, K303R, I304L, A305V, Q306E, Q306H, Q306K, Q306R, L307I, L307M, L307V, S308T, I310F, I310V, I310L, Q311D, Q311S, Q311R, Q311E, G312A, G312T, G312D, G312K, I313V, I313L, E315D, D316G, D316E, D316N, A317G, A317T, K318R, V319L, V319M, I320Y, I320L, V321I, V321L, V321C, S322G, S322V, S322A, S322T, C323I, C323V, C323K, C323P, T324I, T324V, T324L, R325E, R325T, G326D, Y327F, Y327W, Y327V, G328S, K329P, K329R, K329D, K329N, D330E, E331D, P332T, P332R, L333I, L333M, L333F, A334S, A334V, A334C, A334M, K335R, F339M, V341I, L342I, L342M, A343S, A343C, A343V, F344M, F344I, F344V, F344L, F345L, F345M, F345Y, F345V, T346P, Y347F, Y347V, D348E, D348K, D348S, K349G, K349T, K349N, K349S, K349D, W350E, W350I, W350V, W350F, W350L, E351D, I354L, I354C, H355D, H355A, H355Q, H355S, H355E, I356L, I356V, K358R, K358N, T359S, T359A, T359N, T359E, N360S, E362S, E362N, E362T, M363E, I366A, I366K, I366Y, I366L, V370C, V370A, V371S, V371A, I372V, I372M, H373F, H373Y, H373W, S374T, N375H, N375T, N375K, N375R, N375Q, T376D, P377E, P377I, P377V, E378K, E378A, E378D, H379R, H379N, H379V, I380V, E381W, E381H, E381M, E381R, E381K, E381T, A382K, V383I, V383M, A384G, A384S, E385R, E385K, A386Q, A386K, A386R, I387M, I387V, I387L, P388R, P388E, P388Q, P388H, P388D, V389T, V389A, V389C, S390G, S390T, S390C, F392M, F392V, F392I, A393G, V394I, Q396E, Q396D, Q396S, V397W, V397I, V397Y, T400S, T400I, T400H, N401D, N401G, N401S, N401H, N401A, L402M, A405V, A405G, M406W, M406F, M406L, D407H, D407Q, L410M, N411G, N411A, N411P, N411T, T414V, T420S, W421F, N423G, N423R, S425I, S425A, S425G, I426V, S427A, S427T, E428D, T431N, Y433T, Y433H, Y433K, Y433M, Y433R, L435M, L435V, M436V, M436F, M436I, N437Q, L438I, L438T, L438Y, L438C, T439S, R440W, R440K, V441M, V441I, V441L, Y443W, Y443F, Y443R, R444K, P446K, D447E, D447N, M448W, M448K, M448P, Y449R, Y449H, I450V, T452S, T452K, D453H, D453P, E454A, E454D, E455K, E455D, W457F, A458H, A458G, E459D, and any combination thereof.

In some embodiments, an engineered ALDH described herein comprises one or more amino acid alterations selected from the group consisting of: I42V, N119T or N119S, T149I, A158K, S174T, C246A, E259D, A263R, A299R, Y327V, D348S, V370A, F392I, N401A, S427T, and any combination thereof.

In some embodiments, an engineered ALDH described herein includes one or more amino acid alterations described in TABLE 12.

In some embodiments, an engineered ALDH described herein includes an amino acid sequence, other than the one or more amino acid alterations, has at least 65%, at least 70%, at least 75%, at least 80%, at least 85%, at least 90%, at least 95%, at least 98%, at least 99%, or 100% sequence identity to the amino acid sequence referenced in SEQ ID NO: 3.

In some embodiments, described herein is a recombinant nucleic acid encoding an engineered ALDH described herein. The recombinant nucleic acid can include a nucleotide sequence encoding an engineered ALDH operatively linked to a promoter. In some embodiments, described herein is a vector including the recombinant nucleic acid encoding an engineered ALDH described herein and/or a nucleotide sequence encoding an engineered ALDH operatively linked to a promoter.

In some embodiments, described herein is a recombinant nucleic acid encoding: (a) an engineered ACL described herein; and (b) an engineered ADLH described herein. The recombinant nucleic acid can include a nucleotide sequence encoding an engineered ACL operatively linked to a promoter and/or a nucleotide sequence encoding an engineered ACL operatively linked to a first promoter and an engineered ALDH operatively linked to a second promoter.

In some embodiments, described herein is a vector comprising the recombinant nucleic acid encoding: (a) an engineered ACL described herein; and (b) an engineered ADLH described herein. The vector can include a recombinant nucleic acid having a nucleotide sequence encoding an engineered ACL operatively linked to a promoter and/or a nucleotide sequence encoding an engineered ACL operatively linked to a first promoter and an engineered ALDH operatively linked to a second promoter.

In some embodiments, described herein is a non-naturally occurring microbial organism comprising a recombinant nucleic acid encoding an engineered ACL described herein.

In some embodiments, described herein is a non-naturally occurring microbial organism comprising a recombinant nucleic acid encoding an engineered ACL described herein and further comprising a pathway that produces HHA-CoA and/or 1,6 hexanediol (1,6 HDO), or an ester or amide thereof.

In some embodiments, described herein is a non-naturally occurring microbial organism comprising a recombinant nucleic acid encoding an engineered ACL described herein that is capable of producing at least 10% more HHA-CoA and/or 1,6 HDO, or an ester or amide thereof compared to a control microbial organism that does not comprise any one of the nucleic acids encoding an engineered ACL described herein or a nucleic acid comprising a nucleotide sequence encoding an engineered ACL described herein operatively linked to a promoter.

In some embodiments, described herein is a non-naturally occurring microbial organism comprising a recombinant nucleic acid encoding an engineered ALDH described herein.

In some embodiments, described herein is a non-naturally occurring microbial organism of comprising a recombinant nucleic acid encoding an engineered ALDH described herein and further comprising a pathway that produces HHAI and/or 1,6 HDO, or an ester or amide thereof.

In some embodiments, described herein is a non-naturally occurring microbial organism of comprising a recombinant nucleic acid encoding an engineered ALDH described herein that is capable of producing at least 10% more 6-HHAI and/or 1,6 HDO, or an ester or amide thereof compared to a control microbial organism that does not comprise any one of the nucleic acids encoding an engineered ALDH described herein or a nucleic acid comprising a nucleotide sequence encoding an engineered ALDH described herein operatively linked to a promoter.

In some embodiments, described herein is a non-naturally occurring microbial organism comprising a recombinant nucleic acid encoding an engineered ACL described herein or a non-naturally occurring microbial organism comprising a recombinant nucleic acid encoding an engineered ALDH described herein wherein the microbial organism produces a decreased amount of one or more by-product as compared to a control microbial organism that does not comprise a recombinant nucleic acid encoding an engineered ACL described herein or comprising a nucleotide sequence encoding an engineered ACL described herein operatively linked to a promoter, or a recombinant nucleic acid encoding an engineered ALDH described herein or comprising a nucleotide sequence encoding an engineered ALDH described herein operatively linked to a promoter.

In some embodiments, a non-naturally occurring microbial organism described herein having a decreased amount of one or more by-products selected from adipic acid (ADA), 4-HB, 3HB, acetate, succinate, 3-oxoadipate, 3-hydroxyadipate, 2-hexenedioate or combinations thereof.

In some embodiments, a non-naturally occurring microbial organism described herein is capable of producing at least 10% less by-product compared to a control microbial organism that does not comprise a recombinant nucleic acid encoding an engineered ACL described herein or comprising a nucleotide sequence encoding an engineered ACL described herein operatively linked to a promoter, or a recombinant nucleic acid encoding an engineered ALDH described herein or comprising a nucleotide sequence encoding an engineered ALDH described herein operatively linked to a promoter.

In some embodiments, described herein is a non-naturally occurring microbial organism comprising a recombinant nucleic acid encoding (a) an engineered ALDH described herein and (b) an engineered ACL described herein.

In some embodiments, a non-naturally occurring microbial organism described herein comprising a recombinant nucleic acid encoding an engineered ALDH described herein and an engineered ACL described herein further comprises a pathway that produces 6-HHAI and/or 1,6 HDO, or an ester or amide thereof.

In some embodiments, a non-naturally occurring microbial organism described herein comprising a recombinant nucleic acid encoding an engineered ALDH described herein and an engineered ACL described herein, and further comprising a pathway that produces 6-HHAI and/or 1,6 HDO, or an ester or amide thereof is capable of producing at least 10% more HHA-CoA and/or 6-HHAI and/or 1,6 HDO, or an ester or amide thereof compared to a control microbial organism that does not comprise a recombinant nucleic acid encoding an engineered ACL described herein or comprising a nucleotide sequence encoding an engineered ACL described herein operatively linked to a promoter, or a recombinant nucleic acid encoding an engineered ALDH described herein or comprising a nucleotide sequence encoding an engineered ALDH described herein operatively linked to a promoter, or any combination thereof.

In some embodiments, a non-naturally occurring microbial organism described herein comprising a recombinant nucleic acid encoding an engineered ALDH described herein and an engineered ACL described herein, and/or further comprising a pathway that produces 6-HHAI and/or 1,6 HDO, or an ester or amide thereof produces a decreased amount of one or more by-product as compared to a control microbial organism that does not comprise a recombinant nucleic acid encoding an engineered ACL described herein or comprising a nucleotide sequence encoding an engineered ACL described herein operatively linked to a promoter, or a recombinant nucleic acid encoding an engineered ALDH described herein or comprising a nucleotide sequence encoding an engineered ALDH described herein operatively linked to a promoter, or any combination thereof. In some embodiments, the non-naturally occurring microbial organism described herein comprises one or more by-product selected from 4-HB, 3HB, ADA, or both. In some embodiments, non-naturally occurring microbial organism described herein is capable of producing at least 10% less by-product compared to a control microbial organism that does not comprise the recombinant nucleic acid of claim claims 35, 36, 84, 86, or 87.

In some embodiments, any of the non-naturally occurring microbial organisms described herein comprises one or more enzymes of a pathway that is encoded by an exogenous nucleic acid. In some embodiments, the exogenous nucleic acid is heterologous or homologous.

In some embodiments, any of the non-naturally occurring microbial organisms described herein is in a substantially anaerobic culture medium.

In some embodiments, any of the non-naturally occurring microbial organisms described herein is a species of bacteria, yeast, or fungus.

In some embodiments, described herein is a method for producing HHA-CoA and/or 6-HHAI and/or 1,6 HDO, or an ester or amide thereof, comprising culturing any of the non-naturally occurring microbial organisms described herein under conditions and for a sufficient period of time to produce the HHA-CoA and/or 6-HHAI and/or 1,6 HDO, or an ester or amide thereof.

In some embodiments, a method described herein for producing HHA-CoA and/or 6-HHAI and/or 1,6 HDO, or an ester or amide thereof, further comprises separating the HHA-CoA and/or 6-HHAI and/or 1,6 HDO, or an ester or amide thereof from other components in the culture.

In some embodiments, a method described herein for producing HHA-CoA and/or 6-HHAI and/or 1,6 HDO, or an ester or amide thereof, includes separating wherein the separating comprises extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

In some embodiments, described herein is a culture medium comprising the HHA-CoA and/or 6-HHAI and/or 1,6 HDO, or an ester or amide thereof produced by a method described herein, wherein the HHA-CoA and/or 6-HHAI and/or 1,6 HDO, or an ester or amide thereof has a carbon-12, carbon-13 and carbon-14 isotope ratio that reflects an atmospheric carbon dioxide uptake source.

In some embodiments, described herein is a HHA-CoA and/or 6-HHAI and/or 1,6 HDO, or an ester or amide thereof produced according to a method described herein.

In some embodiments, a HHA-CoA and/or 6-HHAI and/or 1,6 HDO, or an ester or amide thereof produced according to a method described herein has an Fm value of at least 80%, at least 85%, at least 90%, at least 95% or at least 98%.

In some embodiments, described herein is a composition comprising a HHA-CoA and/or 6-HHAI and/or 1,6 HDO, or an ester or amide thereof produced according to a method described herein a compound other than the HHA-CoA and/or 6-HHAI and/or 1,6 HDO, or an ester or amide thereof.

In some embodiments, a composition described herein includes a compound other than a HHA-CoA and/or 6-HHAI and/or 1,6 HDO, or an ester or amide thereof that is a trace amount of a cellular portion of a non-naturally occurring microbial organism having a pathway that produces HHA-CoA and/or 6-HHAI and/or 1,6 HDO, or an ester or amide thereof.

In some embodiments, described herein is a composition comprising a HHA-CoA and/or 6-HHAI and/or 1,6 HDO, or an ester or amide thereof produced according to a method described herein and a cell lysate or culture supernatant thereof.

In some embodiments, described is a use of an engineered ACL described herein as a biocatalyst.

In some embodiments, described herein is a composition comprising an engineered ACL described herein and at least one substrate for the engineered ACL. In some embodiments, a composition described herein includes an engineered ACL described herein that can react with a substrate under in vitro conditions. In some embodiments, the substrate is 6-HHA.

In some embodiments, described herein is a use of an engineered ALDH described herein as a biocatalyst.

In some embodiments, described herein is a composition comprising an engineered ALDH described herein and at least one substrate for the engineered ALDH. In some embodiments, a composition described herein includes an engineered ALDH that can react with a substrate under in vitro conditions. In some embodiments, the substrate is HHA-CoA. In some embodiments, the substrate is adipyl-CoA.

### BRIEF DESCRIPTION OF THE DRAWINGS

FIG. 1 shows exemplary pathways from succinyl-CoA and acetyl-CoA to HDO. The enzymes are designated as follows: A) thiolase (Thl), B) a hydroxyadipyl-CoA dehydrogenase (Hbd), C) a crotonase (Crt), D) trans-enoyl CoA reductase (Ter) E) a 6ACA-aldehyde dehydrogenase (6ACA-ALD) F) an alcohol dehydrogenase, G) acyl-coenzyme A ligase (ACL), H) acetylating aldehyde dehydrogenase (ALDH), I) an alcohol dehydrogenase (ADH), and K) carboxylic acid reductase (CAR).

FIG. 2 shows exemplary pathways from succinyl-CoA and acetyl-CoA to HMD. The enzymes are designated as follows: A) 3-oxoadipyl-CoA thiolase; B) 3-oxoadipyl-CoA reductransaminase; C) 3-hydroxyadipyl-CoA dehydratransaminasee; D) adipate semialdehydereductransaminase; E) 3-oxoadipyl-CoA/acyl-CoA transferase; F) 3-oxoadipyl-CoA synthase; G) 3-oxoadipyl-CoA hydrolase; H) 3-oxoadipate reductransaminase; I) 3-hydroxyadipate dehydratransaminasee; J) 5-carboxy-2-pentenoate reductransaminase; K) n adipyl-CoA/acyl-CoA transferase; L) adipyl-CoA synthase; M) adipyl-CoA hydrolase; N) n adipyl-CoA reductransaminase (aldehyde forming); O) 6-aminocaproate transaminase; P) 6-aminocaproate dehydrogenase; Q) 6-aminocaproyl-CoA/acyl-CoA transferase; R) ACL; S) amidohydrolase; T) spontaneous cyclization; U) ALDH; V) HMD transaminase; W) HMD dehydrogenase, X) adipate reductase, Y) adipate kinase, and Z) adipylphosphate reductase.

FIG. 3 shows exemplary pathways to hexamethylenediamine from 6-aminocapropate. Enzymes are A) 6-aminocaproate kinase, B) 6-AHOP oxidoreductase, C) 6-aminocaproic semialdehyde aminotransferase and/or 6-aminocaproic semialdehyde oxidoreductase (aminating), D) 6-aminocaproate N-acetyltransferase, E) 6-acetamidohexanoate kinase, F) 6-AAHOP oxidoreductase, G) 6-acetamidohexanal aminotransferase and/or 6-acetamidohexanal oxidoreductase (aminating), H) 6-acetamidohexanamine N-acetyltransferase and/or 6-acetamidohexanamine hydrolase (amide), I) 6-acetamidohexanoate CoA transferase and/or 6-acetamidohexanoate CoA ligase, J) 6-acetamidohexanoyl-CoA oxidoreductase, K) 6-AAHOP acyltransferase, L) 6-AHOP acyltransferase, M) 6-aminocaproate CoA transferase and/or 6-aminocaproate CoA ligase, N) 6-aminocaproyl-CoA oxidoreductase. Abbreviations are: 6-AAHOP=[(6-acetamidohexanoyl)oxy]phosphonate and 6-AHOP=[(6-aminohexanoyl)oxy]phosphonate.

FIG. 4 shows exemplary biosynthetic pathways leading to 1,6-hexanediol. A) 6-aminocaproyl-CoA transferase or synthetase or ACL; B) 6-aminocaproyl-CoA reductase or ALDH; C) 6-aminocaproate semialdehyde reductase; D) 6-aminocaproate reductase which converts 6ACA to 6-aminocaproate semialdehyde; E) adipyl-CoA reductase; F) adipate semialdehyde reductase; G) 6-hydroxyhexanoyl-CoA transferase or synthetase or ACL; H) 6-hydroxyhexanoyl-CoA reductase or ALDH; I) 6-hydroxyhexanal reductase; J) 6-aminohexanol aminotransferase or oxidoreductases; K) 6-hydroxyhexanoate reductase; L) adipate reductase; and M) adipyl-CoA transferase, hydrolase or synthase.

FIG. 5A shows an in vitro assay for the reaction rate of a template ACL (homolog 1—SEQ ID NO: 1) relative to HHA substrate concentration. The Michaelis-Menten curve for the ACL template derived from homolog 1 (SEQ ID NO: 1) show that both the N-terminal and C-terminal tags yielded sufficient active protein after purification.

FIG. 5B shows an in vitro assay for the reaction rate of a template ACL (homolog 2—SEQ ID NO: 2) relative to HHA substrate concentration. The Michaelis-Menten curve for the ACL template derived from homolog 2 (SEQ ID NO: 2) show that only the N-terminal tag yielded sufficient active protein after purification.

FIG. 6 shows exemplary results of a primary screening of ACL variant activity on HHA in batch replicates. Activity is shown relative to wild-type activity.

FIG. 7A shows an in vitro assay for ACL variants activity on HHA relative to a GFP control after 1 hour.

FIG. 7B shows an in vitro assay for ACL variants activity on HHA relative to a GFP control after 4 hours.

FIG. 8A shows an in vitro assay for the reaction rate of a template ALDH (homolog 3—SEQ ID NO: 3) relative to HHA-CoA substrate concentration. The Michaelis-Menten curve for the ALDH template relative to HHA-CoA show that both the N-terminal and C-terminal tags yielded sufficient active protein after purification.

FIG. 8B shows an in vitro assay for the reaction rate of a template ALDH (homolog 3—SEQ ID NO: 3) relative to adipyl-CoA substrate concentration. The Michaelis-Menten curve shows that only the C-terminal tag yielded sufficient active protein after purification.

FIG. 8C shows an in vitro assay for the reaction rate of a template ALDH (homolog 3—SEQ ID NO: 3) relative to acetyl-CoA substrate concentration. The Michaelis-Menten curve shows that only the C-terminal tag yielded sufficient active protein after purification.

FIG. 8D shows an in vitro assay for the reaction rate of a template ALDH (homolog 3—SEQ ID NO: 3) relative to succinyl-CoA substrate concentration. The Michaelis-Menten curve shows that only the C-terminal tag yielded sufficient active protein after purification.

FIG. 9 shows exemplary results of a primary screening of ALDH homolog 3 variants activity on HHA-CoA over Succinyl-CoA relative to wild-type activity (ALDH homolog 3-SEQ ID NO: 3). Variants were plotted based on two criteria—Criteria 1: Activity on HHA-CoA and Succinyl-CoA, and Criteria 2: Selectivity of HHA-CoA. Variants with increased activity on HHA-CoA relative to the wild-type (homolog 3—SEQ ID NO: 3) with limited activity on Succinyl-CoA were elevated to secondary screening.

FIG. 10 shows an in vitro assay for ALDH variants activity on various substrates.

FIG. 11A shows exemplary results of 1,6 HDO production by the template library as described in Example 7.1 (Library 1). “Control” represents the GFP carrying plasmid and the “Reference” plasmid encodes the best performing pathway.

FIG. 11B shows exemplary results of 1,6 HDO production normalized to OD₆₀₀ by the template library as described in Example 7.1 (Library 1). “Control” represents the GFP carrying plasmid and the “Reference” plasmid encodes the best performing pathway

FIG. 11C shows exemplary results of 4-HB production by the template library as described in Example 7.1 (Library 1). “Control” represents the GFP carrying plasmid and the “Reference” plasmid encodes the best performing pathway

FIG. 11D shows exemplary results of adipic acid production by the template library as described in Example 7.1 (Library 1). “Control” represents the GFP carrying plasmid and the “Reference” plasmid encodes the best performing pathway.

FIG. 12A shows second screen exemplary results of total HDO production by the template library as described in Example 7.2 (Library 1). “Negative” represents the GFP carrying plasmid and the “Positive” represents the plasmid encoding the best performing pathway.

FIG. 12B shows second screen exemplary results of HDO/C6 production by the template library as described in Example 7.2 (Library 1). “Negative” represents the GFP carrying plasmid and the “Positive” represents the plasmid encoding the best performing pathway.

FIG. 13A shows second screen exemplary results of total HDO production by the variant library as described in Example 7.4 (Library 3). “Negative”, “Blank”, “Positive Q6C650”, “Positive Q6N4N8”, “Q6C650+FP” and “Q6N4N8+FP” are controls.

FIG. 13B shows second screen exemplary results of HDO production normalized to OD₆₀₀ by the variant library as described in Example 7.4 (Library 3). “Negative”, “Blank”, “Positive Q6C650”, “Positive Q6N4N8”, “Q6C650+FP” and “Q6N4N8+FP” are controls.

FIG. 14A shows exemplary results of total HDO production by the variant library as compared to the template library in low copy plasmids as described in Example 7.4.

FIG. 14B shows exemplary results of 6-hydroxyhexanoate (HHA) conversion by the variant library as compared to the template library in low copy plasmids as described in Example 7.4.

FIG. 14C shows exemplary results of total HDO production by the variant library as compared to the template library in medium copy plasmids as described in Example 7.4.

FIG. 14D shows exemplary results of 6-hydroxyhexanoate (HHA) conversion by the variant library as compared to the template library in medium copy plasmids as described in Example 7.4.

FIG. 15 shows exemplary HDO production (left) and HDO conversion (right) of varying ACL gene location and RBS strengths.

FIG. 16A shows exemplary results of total HDO production by the library as described in Example 8.1.

FIG. 16B shows exemplary results of HDO conversion by the library as described in Example 8.1.

FIG. 17 shows exemplary results of total HDO production by the library as described in Example 8.2.

FIG. 18 shows exemplary results of total HDO production by the library as described in Example 9.1 with and without TER overexpression.

### DETAILED DESCRIPTION OF THE INVENTION

The subject matter described herein relates to enzyme variants that have desirable properties and are useful for producing desired products, e.g., 6-hydroxyhexanoyl coenzyme-A (interchangeably referred to herein as “HHA-CoA”), 6-hydroxyhexanal, (interchangeably referred to herein as “6-HH”), 1,6 hexanediol (interchangeably referred to herein as “1,6-HDO” or “HDO”) or ester or amide of 1,6-hexanediol, 6-aminocaproyl-CoA (interchangeably referred to herein as “6ACA-CoA”), 6-aminocaproate semialdehyde (interchangeably referred to herein as “6ACA-SADH”), caprolactam (interchangeably referred to herein as “CPO”) and hexamethylenediamine (interchangeably referred to herein as “HMD”). In some embodiments, the subject matter described herein relates to engineered acyl coenzyme-A ligase (interchangeably referred herein as an “ACL”) and/or acetylating aldehyde dehydrogenase (interchangeably referred herein as an “ALDH”), one or both of which are enzyme variants that have markedly different structural and/or functional characteristics compared to a wild-type acyl-coenzyme A ligases or aldehyde dehydrogenase that occurs in nature, respectively. Thus, the engineered acyl coenzyme-A ligases and/or acetylating aldehyde dehydrogenases provided herein are not naturally occurring enzymes. Such engineered acyl coenzyme-A ligases and/or acetylating aldehyde dehydrogenases provided are useful in an engineered cell, such as a microbial organism, that has been engineered to produce a desired product (e.g., HHA-CoA, 6-HH, HDO, an ester or amide of HDO, 6ACA-CoA, 6ACA-SADH, CPO, and/or HMD). For example, as disclosed herein, a cell, such as a microbial organism, having a metabolic pathway can produce a desired product (e.g., HHA-CoA, 6-HH, HDO, an ester or amide of HDO, 6ACA-CoA, 6ACA-SADH, CPO, and/or HMD). Engineered acyl coenzyme-A ligases and/or acetylating aldehyde dehydrogenases having desirable characteristics as described herein can be introduced into a cell, such as microbial organism, that has a metabolic pathway that uses aldehyde dehydrogenase activity to produce a desired product (e.g., HHA-CoA, 6-HH, HDO, an ester or amide of HDO, 6ACA-CoA, 6ACA-SADH, CPO, and/or HMD). Thus, the engineered aldehyde dehydrogenases provided herein can be utilized in engineered cells, such as microbial organisms, to produce a desired product. Such engineered acyl coenzyme-A ligases and/or acetylating aldehyde dehydrogenases are additionally useful as biocatalysts for carrying out desired reactions in vitro. Thus, the engineered acyl coenzyme-A ligases and/or acetylating aldehyde dehydrogenases provided herein can be utilized in engineered cells, such as microbial organisms, to produce a desired product or as an in vitro biocatalyst to produce a desired product.

### Conventions and Abbreviations

| Abbreviation | Convention |
|---|---|
| Ala; A | Alanine |
| Arg; R | Arginine |
| Asn; N | Asparagine |
| Asp; D | Aspartic acid |
| Cys; C | Cysteine |
| Glu; E | Glutamic acid |
| Gln; Q | Glutamine |
| Gly; G | Glycine |
| His; H | Histidine |
| Ile; I | Isoleucine |
| Leu; L | Leucine |
| Lys; K | Lysine |
| Met; M | Methionine |
| Phe; F | Phenylalanine |
| Pro; P | Proline |
| Ser; S | Serine |
| Thr; T | Threonine |
| Trp; W | Tryptophan |
| Tyr; Y | Tyrosine |
| Val; V | Valine |
| 4-HB | 4-hydroxybutyrate |
| 4-HBC | 4-hydroxybutryl-CoA |
| 6-ACA | 6-aminocaproate, also referred |
| | to as 6ACA |
| 6ACA-CoA | 6-aminocaproyl-CoA |
| 6ACA-SADH | 6-aminocaproate semialdehyde |
| 6-HH | 6-hydroxyhexanal, also referred |
| | to as HHAI, or 6-HHAI |
| ACL | Acyl-coenzyme A ligase |
| AcCoA | Acetyl-CoA |
| ADA | Adipic acid |
| ADH | Alcohol dehydrogenase |
| ALD | 6ACA-aldehyde dehydrogenase |
| ALDH | Acetylating aldehyde dehydrogenase |
| CAR | Carboxylic acid reductase |
| CoA | Coenzyme-A |
| CPCoA | 5-carboxy-2-pentenoyl-CoA also |
| | referred to as 2,3-dehydroadipyl-CoA |
| CPO | Caprolactam |
| CRT | Crotonase |
| HDO | 1,6-hexanediol |
| HHA | 6-hydroxyhexanoate, also referred |
| | to as 6-HHA, or 6-HCA |
| HHA-CoA | 6-hydroxyhexanoyl coenzyme-A |
| HBD | hydroxyadipyl-CoA dehydrogenase |
| TER | Transenoyl reductase |
| THL | Thiolase |

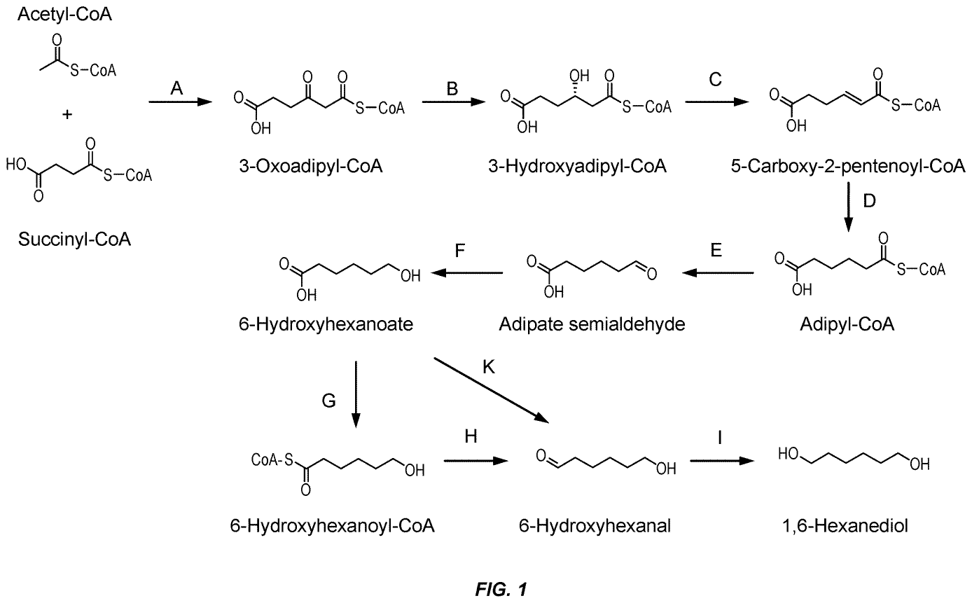

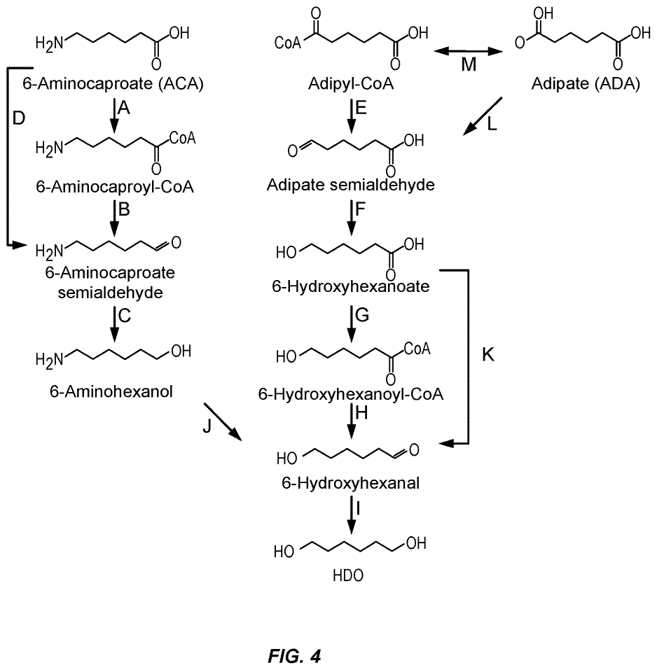

As used herein, “1,6-hexanediol” also referred to as 1,6 HDO, hexane-1,6-diol, hexamethylene glycol, 1,6-dihydroxyhexane, hexamethylenediol, 1,6-hexylene glycol, and 6-hydroxy-1-hexanol; has the chemical structure C₆H₁₄O₂ (see FIGS. 1 and 4 and abbreviated herein as HDO).

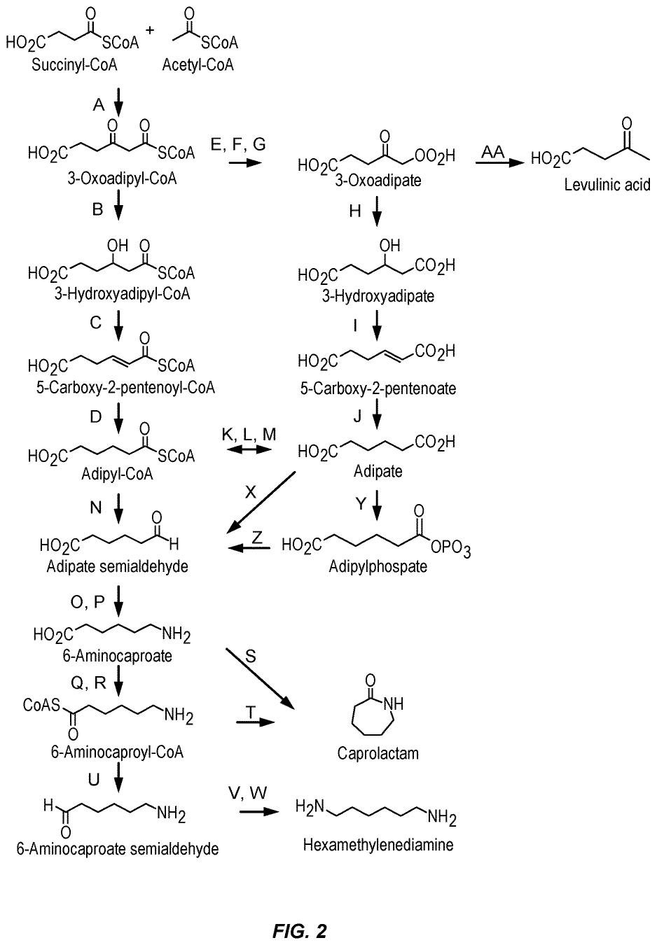

As used herein, “6-aminocaproate,” having the chemical formula —OOC— (CH₂)₅—NH₂ (see FIGS. 2-4, and abbreviated herein as 6-ACA), is the ionized form of 6-aminocaproic acid (IUPAC name 6-aminohexanoic acid), and it is understood that 6-aminocaproate and 6-aminocaproic acid can be used interchangeably throughout to refer to the compound in any of its neutral or ionized forms, including any salt forms thereof. It is understood by those skilled in the art that the specific form will depend on the pH.

As used herein the term “about” means±10% of the stated value. The term “about” can mean rounded to the nearest significant digit. Thus, about 5% means 4.5% to 5.5%. Additionally, about in reference to a specific number also includes that exact number. For example, about 5% also includes exact 5%.

As used herein, the term “alteration” or grammatical equivalents thereof when used in reference to any peptide, polypeptide, protein, nucleic acid or polynucleotide described herein refers to a change in structure of an amino acid residue or nucleic acid base relative to the starting or reference residue or base. An alteration of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. An alteration of a nucleic acid base includes, for example, changing one naturally occurring base for a different naturally occurring base, such as changing an adenine to a thymine or a guanine to a cytosine or an adenine to a cytosine or a guanine to a thymine. An alteration of a nucleic acid base may result in an alteration of the encoding peptide, polypeptide or protein by changing the encoded amino acid residue or function of the peptide, polypeptide or protein. An alteration of a nucleic acid base may not result in an alteration of the amino acid sequence or function of encoded peptide, polypeptide or protein, also known as a silent mutation.

It is understood that when listed herein, alterations listed for a certain position can be in the alternative or in the conjunctive depending on the context. For example, the V, F, FV alterations at a residue corresponding to position 565 in SEQ ID NO: 1, indicates that alterations at a residue corresponding to position 565 in SEQ ID NO: 1 can comprise V or F or FV, wherein FV is F and V. It is also understood that when a lower case “d” is used in reference to an alteration at a specific position, the alteration is a deletion at the specific position.

As used herein, the term “bioderived” means derived from or synthesized by a biological organism and can be considered a renewable resource since it can be generated by a biological organism. Such a biological organism, in particular the non-naturally occurring microbial organism disclosed herein, can utilize feedstock or biomass, such as, sugars (e.g., cellobiose, glucose, fructose, xylose, galactose (e.g., galactose from marine plant biomass), and sucrose), carbohydrates obtained from an agricultural, plant, bacterial, or animal source, and glycerol (e.g., crude glycerol by-product from biodiesel manufacturing) for synthesis of a desired bioderived compound.

As used herein, “caprolactam” (IUPAC name azepan-2-one) is a lactam of 6-aminohexanoic acid (see FIG. 2 and abbreviated herein as CPO).

As used herein, the term “conservative substitution” refers to the replacement of one amino acid for another such that the replacement takes place within a family of amino acids that are related in their side chains. Alternatively, the term “non-conservative substitution” refers to the replacement of one amino acid residue for another such that the replaced residue is going from one family of amino acids to a different family of residues. Genetically encoded amino acids can be divided into four families: (1) acidic (negatively charged)=Asp (D), Glu (G); (2) basic (positively charged)=Lys (K), Arg (R), His (H); (3) non-polar (hydrophobic)=Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic=Ala (A), Val (V), Leu (L), Ile (I), Met (M), Phe (F); and (ii) moderately hydrophobic=Gly (G), Pro (P), Cys (C), Tyr (Y), Trp (W); and (4) uncharged polar=Asn (N), Gln (Q), Ser (S), Thr (T). In alternative fashion, the amino acid repertoire can be grouped as (1) acidic (negatively charged)=Asp (D), Glu (G); (2) basic (positively charged)=Lys (K), Arg (R), His (H), and (3) aliphatic=Gly (G), Ala (A), Val (V), Leu (L), Ile (I), Ser (S), Thr (T), with Ser (S) and Thr (T) optionally being grouped separately as aliphatic-hydroxyl; (4) aromatic=Phe (F), Tyr (Y), Trp (W); (5) amide=Asn (N), Glu (Q); and (6) sulfur-containing=Cys (C) and Met (M) (see, for example, Biochemistry, 4th ed., Ed. by L. Stryer, WH Freeman and Co., 1995, which is incorporated by reference herein in its entirety).

As used herein, the term “culture medium,” “medium,” “growth medium” or grammatical equivalents thereof refers to a liquid or solid (e.g., gelatinous) substance containing nutrients that support the growth of a cell, including a microbial organism, such as the microbial organism described herein. Nutrients that support growth include, but are not limited to, the following: a substrate that supplies carbon, such as, but are not limited to, cellobiose, galactose, glucose, xylose, ethanol, acetate, arabinose, arabitol, sorbitol and glycerol; salts that provide essential elements including magnesium, nitrogen, phosphorus, and sulfur; a source for amino acids, such as peptone or tryptone; and a source for vitamin content, such as yeast extract. Culture medium can be a defined medium, in which quantities of all ingredients are known, or an undefined medium, in which the quantities of all ingredients are not known. Culture medium can also include substances other than nutrients needed for growth, such as a substance that only allows select cells to grow (e.g., antibiotic or antifungal), which are generally found in selective medium, or a substance that allows for differentiation of one microbial organism over another when grown on the same medium, which are generally found in differential or indicator medium. Such substances are well known to a person skilled in the art.

As used herein, the term “engineered” or “variant” when used in reference to any peptide, polypeptide, protein, nucleic acid or polynucleotide described herein refers to a sequence of amino acids or nucleic acids having at least one alteration at an amino acid residue or nucleic acid base as compared to a parent sequence. Such a sequence of amino acids or nucleic acids is not naturally occurring. The parent sequence of amino acids or nucleic acids can be, for example, a wild-type sequence or a homolog thereof, or a modified variant of a wild-type sequence or homolog thereof.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid described herein can utilize either or both a heterologous or homologous encoding nucleic acid.

It is understood that, when more than one recombinant nucleic acid and/or exogenous nucleic acid is included into a microbial organism, the more than one recombinant nucleic acid and/or exogenous nucleic acid refers to the referenced encoding nucleic acid or biosynthetic activity, as discussed herein. It is further understood, as disclosed herein, that such more than one recombinant nucleic acids or exogenous nucleic acids can be introduced into the host microbial organism on separate nucleic acid molecules, on polycistronic nucleic acid molecules, or a combination thereof, and still be considered as more than one recombinant nucleic acid and/or exogenous nucleic acid. For example, as disclosed herein a microbial organism can be engineered to express two or more recombinant and/or exogenous nucleic acids encoding a desired pathway enzyme or protein. In the case where two recombinant and/or exogenous nucleic acids encoding an enzyme or protein having a desired activity are introduced into a host microbial organism, it is understood that the two recombinant and/or exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two recombinant and/or exogenous nucleic acids can be introduced into a host organism in any desired combination, for example, on a single plasmid, on separate plasmids, can be integrated into the host chromosome at a single site or multiple sites, and still be considered as two or more recombinant or exogenous nucleic acids, for example three exogenous nucleic acids. Thus, the number of referenced recombinant or exogenous nucleic acids or biosynthetic activities refers to the number of encoding nucleic acids or the number of biosynthetic activities, not the number of separate nucleic acids introduced into the host organism.

The term “Fm value” or “Fraction Modern value” when used in reference to a compound is a ratio of carbon-14 (¹⁴C) to carbon-12 (¹²C). Specifically, Fm value is computed from the expression: Fm=(S−B)/(M−B), where B, S and M represent the ¹⁴C/¹²C ratios of the blank, the sample and the modern reference, respectively. Fm value is a measurement of the deviation of the ¹⁴C/¹²C ratio of a sample from “Modem.” Modern is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil (Olsson, The use of Oxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950)¹⁴C/¹²C ratio of 1.176±0.010×10⁻¹² (Karlen et al., Arkiv Geofysik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for δ¹³. An Fm=0% represents the entire lack of carbon-14 atoms in a material, thus indicating a fossil (for example, petroleum based) carbon source, whereas a Fm=100%, after correction for the post-1950 injection of carbon-14 into the atmosphere from nuclear bomb testing, indicates an entirely modern carbon source. The percent modern carbon (pMC) can be greater than 100% because of the continuing but diminishing effects of the 1950s nuclear testing programs, which resulted in a considerable enrichment of carbon-14 in the atmosphere. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old. Applications of carbon-14 dating techniques to quantify bio-based content of materials are well known in the art (see, e.g., Currie et al., Nuclear Instruments and Methods in Physics Research B, 172:281-287 (2000), and Colonna et al., Green Chemistry, 13:2543-2548 (2011)).

As used herein, the term “functional fragment” when used in reference to a peptide, polypeptide or protein is intended to refer to a portion of the peptide, polypeptide or protein that retains some or all of the activity (e.g., catalyzing the conversion of HHA to HHA-CoA, the conversion of HHA-CoA to 6-HHAI, the conversion of 6-ACA to 6ACA-CoA, and/or the conversion of 6ACA-CoA to 6ACA-SADH) of the original peptide, polypeptide or protein from which the fragment was derived. Such functional fragments include amino acid sequences that are about 200 to about 460, about 200 to about 450, about 200 to about 440, about 200 to about 430, about 200 to about 420, about 200 to about 410, about 200 to about 400, about 200 to about 390, about 200 to about 380, about 200 to about 370, about 200 to about 360, about 200 to about 350, about 300 to about 460, about 300 to about 450, about 300 to about 440, about 300 to about 430, about 300 to about 420, about 300 to about 410, about 300 to about 400, about 300 to about 390, about 300 to about 380, about 300 to about 370, about 300 to about 350, about 300 to about 340, about 300 to about 330, about 300 to about 320, about 300 to about 310, about 400 to about 460, about 400 to about 450, about 400 to about 440, about 400 to about 430, about 400 to about 420, about 400 to about 410, about 450 to about 460 amino acids in length. These functional fragments can, for example, be truncations (e.g., C-terminal or N-terminal truncations) of a peptide, polypeptide, or protein. Functional fragments can also include one or more amino acid alteration described herein, such as an amino acid alteration of an engineered peptide described herein.

As used herein, the term “isolated” when used in reference to a molecule (e.g., peptide, polypeptide, protein, nucleic acid, polynucleotide, vector) or a cell (e.g., a yeast cell) refers to a molecule or cell that is substantially free of at least one component with which the referenced molecule or cell is found in nature. The term includes a molecule or cell that is removed from some or all components with which it is found in its natural environment. Therefore, an isolated molecule or cell can be partly or completely separated from other substances with which it is found in nature or with which it is grown, stored or subsisted in non-naturally occurring environments.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “non-naturally occurring” when used in reference to a microbial organism described herein is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, genetic alterations within coding regions and functional fragments thereof. Additional modifications include, for example, non-coding regulatory regions in which the modifications alter expression of a gene or operon. Exemplary metabolic polypeptides include enzymes or proteins within a 1,6-HDO, HMD or other bioderived compound pathway described herein.

As use herein, the term “operatively linked” when used in reference to a nucleic acid encoding an engineered ACL and/or ALDH refers to connection of a nucleotide sequence encoding an engineered ACL and/or ALDH described herein to another nucleotide sequence (e.g., a promoter) is such a way as to allow for the connected nucleotide sequences to function (e.g., express the engineered ACL and/or ALDH in the microbial organism).

As used herein, the term “pathway” when used in reference to production of a desired product (e.g., HHA-CoA, 6-HHAI, HDO, an ester or amide of HDO, 6ACA-CoA, 6ACA-SADH, CPO, and/or HMD) refers to one or more polypeptides (e.g., proteins or enzymes) that catalyze the conversion of a substrate compound to a product compound and/or produce a co-substrate for the conversion of a substrate compound to a product compound. Such a product compound can be one of the bioderived compounds described herein, or an intermediate compound that can lead to the bioderived compound upon further conversion by other proteins or enzymes of the metabolic pathway. Accordingly, a metabolic pathway can be comprised of a series of metabolic polypeptides (e.g., two, three, four, five, six, seven, eight, nine, ten or more) that act upon a substrate compound to convert it to a given product compound through a series of intermediate compounds. The metabolic polypeptides of a metabolic pathway can be encoded by an exogenous nucleic acid as described herein or produced naturally by the host microbial organism.

As used herein, the term “recombinant” with respect to a nucleic acid, such as a nucleic acid comprising a gene that encodes a protein or polypeptide (e.g., an engineered ACL and/or ALDH described herein), refers to: a nucleic acid that has been artificially supplied to a biological system; a nucleic acid that has been modified within a biological system, or a nucleic acid whose expression or regulation has been manipulated within a biological system. The recombinant nucleic acid can be supplied to the biological system, for example, by introduction of the nucleic acid into genetic material of a microbial organism, such as by integration into a microbial organism chromosome, or as non-chromosomal genetic material such as a plasmid. A recombinant nucleic acid that is introduced into or expressed in a microbial organism may be a nucleic acid that comes from a different organism or species from the microbial organism, or may be a synthetic nucleic acid, or may be a nucleic acid that is also endogenously expressed in the same organism or species as the microbial organism. A recombinant nucleic acid that is also endogenously expressed in the same organism or species as the microbial organism can be considered heterologous if: the sequence of the recombinant nucleic acid is modified relative to the endogenously expressed sequence, the sequence of a regulatory region such as a promoter that controls expression of the nucleic acid is modified relative to the regulatory region of the endogenously expressed sequence, the nucleic acid is expressed in an alternate location in the genome of the microbial organism relative to the endogenously expressed sequence, the nucleic acid is expressed in a different copy number in the microbial organism relative to the endogenously expressed sequence, and/or the nucleic acid is expressed as non-chromosomal genetic material such as a plasmid in the microbial organism.

As used herein, the term “promoter” when used in reference to a nucleic acid encoding an engineered ACL and/or ALDH refers to a nucleotide sequence where transcription of a linked open reading frame (e.g., a nucleotide sequence encoding an engineered ACL and/or ALDH) by an RNA polymerase begins. A promoter sequence can be located directly upstream or at the 5′ end of the transcription initiation site. RNA polymerase and the necessary transcription factors bind to a promoter sequence and initiate transcription. Promoter sequences define the direction of transcription and indicate which DNA strand will be transcribed, i.e. the sense strand.

As used herein, the term “substantially anaerobic” when used in reference to a culture or growth condition is intended to mean that the amount of dissolved oxygen in a liquid medium is less than about 10% of saturation. The term also is intended to include sealed chambers maintained with an atmosphere of less than about 1% oxygen that include liquid or solid medium.

As used herein, the term “vector” refers to a compound and/or composition that transduces, transforms, or infects a microbial organism, thereby causing the microbial organism to express nucleic acids and/or proteins other than those native to the microbial organism, or in a manner not native to the cell. Vectors can be constructed to include one or more biosynthetic pathway enzyme or protein, such as an engineered ACL and/or ALDH described herein, encoded by a nucleotide sequence operably linked to expression control sequences (e.g., promoter) that are functional in the microbial organism (“expression vector”). Expression vectors applicable for use in the microbial organisms described herein include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more recombinant or exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of a recombinant or exogenous nucleic acid encoding an enzyme or protein involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid or its corresponding gene product (e.g., enzyme or protein). It is understood by those skilled in the art that the recombinant or exogenous nucleic acid is expressed in a sufficient amount to produce the desired product, and it is further understood that expression levels can be optimized to obtain sufficient expression using methods well known in the art and as disclosed herein.

Those skilled in the art will understand that the genetic alterations, including metabolic modifications exemplified herein, are described with reference to a suitable microbial organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. For example, genes that are orthologs can encode proteins with sequence similarity of about 25%, 30%, 35%, 40%, 45%, 50%, 55%, 60%, 65%, 70%, 75%, 80%, 85%, 90%, 95%, or 100% amino acid sequence identity. Genes encoding proteins sharing an amino acid similarity less than 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microbial organism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease and the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms described herein having biosynthetic capability for a desired product, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microbial organism that encode an enzyme catalyzing a similar or substantially similar metabolic reaction, those skilled in the art also can utilize these evolutionally related genes. Similarly, for a gene disruption, evolutionally related genes can also be disrupted or deleted in a microbial organism to reduce or eliminate functional redundancy of enzymatic activities targeted for disruption.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. For example, related gene products or proteins can be expected to have about 25%, 30%, 35%, 40%, 45%, 50%, 55%, 60%, 65%, 70%, 75%, 80%, 85%, 90%, 95%, or 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleotide sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

An engineered ACL described herein converts an acid into its corresponding acyl-CoA. Such an enzyme can also be referred to as acid-thiol ligase that converts an acid into acyl-CoA. Such an engineered ACL described herein can be classified as a reaction 6.2.1-, acid-thiol ligase (acid to acyl-CoA), where the first three digits correspond to the first three Enzyme Commission number digits which denote the general type of transformation independent of substrate specificity (e.g., EC 6.2.1.-). Exemplary enzymatic conversions of an engineered ACL provided herein include, but are not limited to, the conversion of HHA to HHA-CoA, and the conversion of 6-ACA to 6ACA-CoA. An ACL described herein can be used to produce desired products, such as HHA-CoA, 1,6-HDO, 6ACA-CoA, HMD, or other desired products such as a downstream product, including an ester or amide thereof, in a cell, such as a microbial organism, containing a suitable metabolic pathway, or in vitro.

An engineered ALDH described herein converts an acyl-CoA to its corresponding aldehyde. Such an enzyme can also be referred to as an oxidoreductase that converts an acyl-CoA to its corresponding aldehyde. Such an engineered ALDH described herein can be classified as a reaction 1.2.1.-, oxidoreductase (acyl-CoA to aldehyde), where the first three digits correspond to the first three Enzyme Commission number digits which denote the general type of transformation independent of substrate specificity (e.g., EC 1.2.1.-). Exemplary enzymatic conversions of an engineered ALDH provided herein include, but are not limited to, the conversion of HHA-CoA to 6-HH, and the conversion of 6ACA-CoA to 6ACA-SADH. An ALDH described herein can be used to produce desired products, such as 6-HH, 1,6-HDO, 6ACA-SADH, HMD, or other desired products such as a downstream product, including an ester or amide thereof, in a cell, such as a microbial organism, containing a suitable metabolic pathway, or in vitro.

ACLs and/or ALDHs described herein are particularly useful to provide an improved enzymatic route and microorganism to provide an improved composition of HDO and/or HMD, and further having improved purity qualities with respect to by-products.

### Acyl Coenzyme-A Ligase (ACL)

In some embodiments, provided herein is an engineered ACL that is a variant of a wild-type ACL (e.g., SEQ ID NOS: 1 and 2). Such an engineered ACL includes one or more alterations at a position described in TABLE 8 or TABLE 9, and, in some embodiments, a combination of alterations described in TABLE 8 or TABLE 9, and has higher activity relative to the WT ACL (e.g., SEQ ID NOS: 1 or 2) as described herein. In certain embodiments, provided herein is an engineered ACL that is a variant of a WT ACL (e.g., SEQ ID NO: 1). Such an engineered ACL includes one or more alterations at a position described in TABLE 8, and, in some embodiments, a combination of alterations described in TABLE 8, and has higher activity relative to the WT ACL (e.g., SEQ ID NO: 1) as described herein. In certain embodiments, provided herein is an engineered ACL that is a variant of a WT ACL (e.g., SEQ ID NO: 2). Such an engineered ACL includes one or more alterations at a position described in TABLE 9, and, in some embodiments, a combination of alterations described in TABLE 9, and has higher activity relative to the WT ACL (e.g., SEQ ID NO: 2) as described herein. In some embodiments, the term “activity” as used herein to describe enzymatic activity, refers to catalytic activity, substrate specificity, substrate affinity, or any combination thereof.

In some embodiments, an engineered ACL provided herein is capable of catalyzing the conversion of: 1) 6-hydroxyhexanoate (HHA) to 6-hydroxyhexanoyl-CoA (HHA-CoA); 2) adipate to adipyl-CoA, 3) succinate to succinyl-CoA, 4) propionate to propionyl-CoA, 5) acetate to acetyl-CoA, 6) 4-hydroxybutyrate (4-HB) to 4-hydroxybutyryl-CoA (4-HBC), 7) 6-ACA to 6-ACA-CoA, 8) 3-hydroxybutyrate (3HB) to 3-hydroxybutyryl-CoA (3HIB-CoA), 9) 3HB to either isomer of 3HB-CoA, or combinations thereof. Accordingly, in some embodiments, an engineered ACL provided herein is capable of catalyzing the conversion of 6-hydroxyhexanoate to 6-hydroxyhexanoyl-CoA. In some embodiments, an engineered ACL provided herein is capable of catalyzing the conversion of adipate to adipyl-CoA. In some embodiments, an engineered ACL provided herein is capable of catalyzing the conversion of succinate to succinyl-CoA. In some embodiments, an engineered ACL provided herein is capable of catalyzing the conversion of propionate to propionyl-CoA. In some embodiments, an engineered ACL provided herein is capable of catalyzing the conversion of acetate to acetyl-CoA. In some embodiments, an engineered ACL provided herein is capable of catalyzing the conversion of 4-hydroxybutyrate (4-HB) to 4-hydroxybutyryl-CoA (4-HBC). In some embodiments, an engineered ACL provided herein is capable of catalyzing the conversion of 6-ACA to 6ACA-CoA. In some embodiments, an engineered ACL provided herein is capable of catalyzing the conversion of 3HB to 3HB-CoA. In some embodiments, an engineered ACL provided herein is capable of catalyzing the conversion of 3HB to either isomer of 3HB-CoA.

In some embodiments, an engineered ACL as described herein has higher catalytic activity in the conversion of select substrates over other substrates. For example, in some embodiments, an engineered ACL as described herein has: 1) higher specificity for conversion of HHA to HHA-CoA over conversion of adipate to adipyl-CoA, 2) higher specificity for conversion of HHA to HHA-CoA over conversion of succinate to succinyl-CoA, 3) higher specificity for conversion of HHA to HHA-CoA over conversion of propionate to propionyl-CoA, 4) higher specificity for conversion of HHA to HHA-CoA over conversion of acetate to acetyl-CoA, 5) higher specificity for conversion of HHA to HHA-CoA over conversion of 4-hydroxybutyrate (4-HB) to 4-hydroxybutyryl-CoA (4-HBC), 6) higher specificity for conversion of HHA to HHA-CoA over the conversion of 3HB to 3HB-CoA, and 7) higher specificity for conversion of HHA to HHA-CoA over the conversion of 3HB to either isomer of 3HB-CoA, or any combination thereof. In another example, in some embodiments, an engineered ACL as described herein has: 1) higher specificity for conversion of 6-ACA to 6ACA-CoA over conversion of adipate to adipyl-CoA, 2) higher specificity for conversion of 6-ACA to 6ACA-CoA over conversion of succinate to succinyl-CoA, 3) higher specificity for conversion of 6-ACA to 6ACA-CoA over conversion of propionate to propionyl-CoA, 4) higher specificity for conversion of 6-ACA to 6ACA-CoA over conversion of acetate to acetyl-CoA, 5) higher specificity for conversion of 6-ACA to 6ACA-CoA over conversion of 4-hydroxybutyrate (4-HB) to 4-hydroxybutyryl-CoA (4-HBC), 6) higher specificity for conversion of 6-ACA to 6ACA-CoA over the conversion of 3HB to 3HB-CoA, and 7) higher specificity for conversion of 6-ACA to 6ACA-CoA over the conversion of 3HB to either isomer of 3HB-CoA, or any combination thereof. Accordingly, in some embodiments, an engineered ACL as described herein has: 1) higher specificity for conversion of HHA to HHA-CoA and/or 6-ACA to 6ACA-CoA over conversion of adipate to adipyl-CoA, 2) higher specificity for conversion of HHA to HHA-CoA and/or 6-ACA to 6ACA-CoA over conversion of succinate to succinyl-CoA, 3) higher specificity for conversion of HHA to HHA-CoA and/or 6-ACA to 6ACA-CoA over conversion of propionate to propionyl-CoA, 4) higher specificity for conversion of HHA to HHA-CoA and/or 6-ACA to 6ACA-CoA over conversion of acetate to acetyl-CoA, 5) higher specificity for conversion of HHA to HHA-CoA and/or 6-ACA to 6ACA-CoA over conversion of 4-hydroxybutyrate (4-TB) to 4-hydroxybutyryl-CoA (4-H1BC), 6) higher specificity for conversion of HHA to HHA-CoA and/or 6-ACA to 6ACA-CoA over the conversion of 3HB to 3HB-CoA, and 7) higher specificity for conversion of HHA to HHA-CoA and/or 6-ACA to 6ACA-CoA over the conversion of 3HB to either isomer of 3HB-CoA, or any combination thereof

In some embodiments, provided herein is an engineered ACL comprising a variant of amino acid sequence SEQ ID NO: 1, wherein the engineered ACL comprises one or more alterations at a position described in TABLE 8. In some embodiments, such an engineered ACL is capable of catalyzing the conversion of: 1) 6-hydroxyhexanoate (HHA) to 6-hydroxyhexanoyl-CoA (HHA-CoA); 2) adipate to adipyl-CoA, 3) succinate to succinyl-CoA, 4) propionate to propionyl-CoA, 5) acetate to acetyl-CoA, 6) 4-hydroxybutyrate (4-Tm) to 4-hydroxybutyryl-CoA (4-HBC), 7) 6-ACA to 6-ACA-CoA, 8) 3HB to 3HB-CoA, 9) 3HB to either isomer of 3HB-CoA, or combinations thereof. Accordingly, in some embodiments, such an engineered ACL comprising a variant of amino acid sequence SEQ ID NO: 1 is capable of catalyzing the conversion of 6-hydroxyhexanoate to 6-hydroxyhexanoyl-CoA. In some embodiments, an engineered ACL comprising a variant of amino acid sequence SEQ ID NO: 1 is capable of catalyzing the conversion of adipate to adipyl-CoA. In some embodiments, an engineered ACL comprising a variant of amino acid sequence SEQ ID NO: 1 is capable of catalyzing the conversion of succinate to succinyl-CoA. In some embodiments, an engineered ACL comprising a variant of amino acid sequence SEQ ID NO: 1 is capable of catalyzing the conversion of propionate to propionyl-CoA. In some embodiments, an engineered ACL comprising a variant of amino acid sequence SEQ ID NO: 1 is capable of catalyzing the conversion of acetate to acetyl-CoA. In some embodiments, an engineered ACL comprising a variant of amino acid sequence SEQ ID NO: 1 is capable of catalyzing the conversion of 4-hydroxybutyrate (4-Tm) to 4-hydroxybutyryl-CoA (4-TmC). In some embodiments, an engineered ACL comprising a variant of amino acid sequence SEQ ID NO: 1 is capable of catalyzing the conversion of 6-ACA to 6ACA-CoA. In some embodiments, an engineered ACL comprising a variant of amino acid sequence SEQ ID NO: 1 is capable of catalyzing the conversion of 3Tm to 3HB-CoA. In some embodiments, an engineered ACL comprising a variant of amino acid sequence SEQ ID NO: 1 is capable of catalyzing the conversion of 3Tm to either isomer of 3HB-CoA.

In some embodiments, provided herein is an engineered ACL comprising a variant of amino acid sequence SEQ ID NO: 2, wherein the engineered ACL comprises one or more alterations at a position described in TABLE 9. In some embodiments, such an engineered ACL is capable of catalyzing the conversion of: 1) 6-hydroxyhexanoate (HHA) to 6-hydroxyhexanoyl-CoA (HHA-CoA); 2) adipate to adipyl-CoA, 3) succinate to succinyl-CoA, 4) propionate to propionyl-CoA, 5) acetate to acetyl-CoA, 6) 4-hydroxybutyrate (4-Tm) to 4-hydroxybutyryl-CoA (4-TmC), 7) 6-ACA to 6-ACA-CoA, 8) 3HB to 3HB-CoA, 9) 3Tm to either isomer of 3HB-CoA, or combinations thereof. Accordingly, in some embodiments, such an engineered ACL comprising a variant of amino acid sequence SEQ ID NO: 2 is capable of catalyzing the conversion of 6-hydroxyhexanoate to 6-hydroxyhexanoyl-CoA. In some embodiments, an engineered ACL comprising a variant of amino acid sequence SEQ ID NO: 2 is capable of catalyzing the conversion of adipate to adipyl-CoA. In some embodiments, an engineered ACL comprising a variant of amino acid sequence SEQ ID NO: 2 is capable of catalyzing the conversion of succinate to succinyl-CoA. In some embodiments, an engineered ACL comprising a variant of amino acid sequence SEQ ID NO: 2 is capable of catalyzing the conversion of propionate to propionyl-CoA. In some embodiments, an engineered ACL comprising a variant of amino acid sequence SEQ ID NO: 2 is capable of catalyzing the conversion of acetate to acetyl-CoA. In some embodiments, an engineered ACL comprising a variant of amino acid sequence SEQ ID NO: 2 is capable of catalyzing the conversion of 4-hydroxybutyrate (4-TB) to 4-hydroxybutyryl-CoA (4-HBC). In some embodiments, an engineered ACL comprising a variant of amino acid sequence SEQ ID NO: 2 is capable of catalyzing the conversion of 6-ACA to 6ACA-CoA. In some embodiments, an engineered ACL comprising a variant of amino acid sequence SEQ ID NO: 2 is capable of catalyzing the conversion of 3HB to 3HB-CoA. In some embodiments, an engineered ACL comprising a variant of amino acid sequence SEQ ID NO: 2 is capable of catalyzing the conversion of 3HB to either isomer of 3HB-CoA.

It is understood that the engineered ACLs as described herein can carry out a similar enzymatic reaction as the wild-type ACLs (SEQ ID NO: 1 or 2) as discussed above. It is further understood that the variants of the ACL enzyme can include alterations that provide a beneficial characteristic to the engineered ACL, including but not limited to, increased activity (e.g., ability to catalyze a reaction described herein and/or selectivity for a substrate, such as HHA or 6-ACA) as described herein (see, e.g., Example 2 and 4). In some embodiments, the engineered ACL can exhibit an activity that is at least the same or higher than the wild-type ACL (SEQ ID NO: 1 or 2), that is, it has activity that is the same or higher than an ACL without the variant at the same amino acid position(s). In some embodiments, the engineered ACL can exhibit two or more activities (e.g., ability to catalyze a reaction described herein and selectivity for a substrate, such as HHA or 6-ACA) that are at least the same or higher than the wild-type ACL (SEQ ID NO: 1 or 2), that is, it has two or more activities that are the same or higher than an ACL without the variant at the same amino acid position(s). For example, the engineered ACLs provided here can have one or more activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher over a wild-type ACL (see, e.g., Example 2 and 4). In some embodiments, an engineered ACL provided herein has an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an ACL consisting of the amino acid sequence of SEQ ID NO: 1. In some embodiments, an engineered ACL provided herein has an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an ACL consisting of the amino acid sequence of SEQ ID NO: 2. In some embodiments, an engineered ACL provided herein has an activity that is at least 10% higher. In some embodiments, an engineered ACL provided herein has an activity that is at least 20% higher. In some embodiments, an engineered ACL provided herein has an activity that is at least 30% higher. In some embodiments, an engineered ACL provided herein has an activity that is at least 40% higher. In some embodiments, an engineered ACL provided herein has an activity that is at least 50% higher. In some embodiments, an engineered ACL provided herein has an activity that is at least 60% higher. In some embodiments, an engineered ACL provided herein has an activity that is at least 70% higher. In some embodiments, an engineered ACL provided herein has an activity that is at least 80% higher. In some embodiments, an engineered ACL provided herein has an activity that is at least 90% higher. In some embodiments, an engineered ACL provided herein has an activity that is at least 100% higher. In some embodiments, an engineered ACL provided herein has an activity that is at least 110% higher. In some embodiments, an engineered ACL provided herein has an activity that is at least 120% higher. In some embodiments, an engineered ACL provided herein has an activity that is at least 130% higher. In some embodiments, an engineered ACL provided herein has an activity that is at least 140% higher. In some embodiments, an engineered ACL provided herein has an activity that is at least 150% higher. In some embodiments, an engineered ACL provided herein has an activity that is at least 160% higher. In some embodiments, an engineered ACL provided herein has an activity that is at least 170% higher. In some embodiments, an engineered ACL provided herein has an activity that is at least 180% higher. In some embodiments, an engineered ACL provided herein has an activity that is at least 190% higher. In some embodiments, an engineered ACL provided herein has an activity that is at least 200% higher. It is understood that activity refers to the ability of an engineered ACL described herein to convert a substrate to a product relative to a wild-type ACL (SEQ ID NO: 1 or 2) under the same assay conditions, such as those described herein (see, e.g., Examples 2 and 4).

In some embodiments, the activity of an ACL described herein is measured as the catalytic constant (k_{cat}) value or turnover number. In some embodiments, the k_{cat} is at least 0.1 s⁻¹, at least 0.2 s⁻¹, at least 0.3 s⁻¹, at least 0.4 s⁻¹, at least 0.5 s⁻¹, at least 0.6 s⁻¹, at least 0.7 s⁻¹, at least 0.8 s⁻¹, at least 0.9 s⁻¹, at least 1 s⁻¹, at least 2 s⁻¹, at least 3 s⁻¹, at least 4 s⁻¹, at least 5 s⁻¹, at least 6 s⁻¹, at least 7 s⁻¹, at least 8 s⁻¹, at least 9 s⁻¹, at least 10 s⁻¹, at least 11 s⁻¹, at least 12 s⁻¹, at least 13 s⁻¹, at least 14 s⁻¹, at least 15 s⁻¹, at least 16 s⁻¹, at least 17 s⁻¹, at least 18 s⁻¹, at least 19 s⁻¹, at least 20 s⁻¹, at least 21 s⁻¹, at least 22 s⁻¹, at least 23 s⁻¹, at least 24 s⁻¹, at least 25 s⁻¹, at least 26 s⁻¹, at least 27 s⁻¹, at least 28 s⁻¹, at least 29 s⁻¹, at least 30 s⁻¹, at least 31 s⁻¹, at least 32 s⁻¹, at least 33 s⁻¹, at least 34 s⁻¹, at least 35 s⁻¹, at least 36 s⁻¹, at least 37 s⁻¹, at least 38 s⁻¹, at least 39 s⁻¹, at least 40 s⁻¹, at least 41 s⁻¹, at least 42 s⁻¹, at least 43 s⁻¹, at least 44 s⁻¹, at least 45 s⁻¹, at least 46 s⁻¹, at least 47 s⁻¹, at least 48 s⁻¹, at least 49 s⁻¹, at least 50 s⁻¹, at least 51 s⁻¹, at least 52 s⁻¹, at least 53 s⁻¹, at least 54 s⁻¹, at least 55 s⁻¹, at least 56 s⁻¹, at least 57 s⁻¹, at least 58 s⁻¹, at least 59 s⁻¹, at least 60 s⁻¹, at least 61 s⁻¹, at least 62 s⁻¹, at least 63 s⁻¹, at least 64 s⁻¹, at least 65 s⁻¹, at least 66 s⁻¹, at least 67 s⁻¹, at least 68 s⁻¹, at least 69 s⁻¹, at least 70 s⁻¹, at least 71 s⁻¹, at least 72 s⁻¹, at least 73 s⁻¹, at least 74 s⁻¹, at least 75 s⁻¹, at least 76 s⁻¹, at least 77 s⁻¹, at least 78 s⁻¹, at least 79 s⁻¹, at least 80 s⁻¹, at least 81 s⁻¹, at least 82 s⁻¹, at least 83 s⁻¹, at least 84 s⁻¹, at least 85 s⁻¹, at least 86 s⁻¹, at least 87 s⁻¹, at least 88 s⁻¹, at least 89 s⁻¹, at least 90 s⁻¹, at least 91 s⁻¹, at least 92 s⁻¹, at least 93 s⁻¹, at least 94 s⁻¹, at least 95 s⁻¹, at least 96 s⁻¹, at least 97 s⁻¹, at least 98 s⁻¹, at least 99 s⁻¹, at least 100 s⁻¹, at least 500 s⁻¹, or at least 1000 s⁻¹. In some embodiments, the K_{cat} is between 1 s⁻¹ and 100 s⁻¹, between 5 s⁻¹ and 50 s⁻¹, or between 10 s⁻¹ and 50 s⁻¹.

In some embodiments, the activity of an ACL described herein is measured as the Michaelis constant (Kₘ). In some embodiments, the Kₘ is less than 0.1 μM, less than 0.2 μM, less than 0.3 μM, less than 0.4 μM, less than 0.5 μM, less than 0.6 μM, less than 0.7 μM, less than 0.8 μM, less than 0.9 μM, less than 1 μM, less than 2 μM, less than 3 μM, less than 4 μM, less than 5 μM, less than 6 μM, less than 7 μM, less than 8 μM, less than 9 μM, less than 10 μM, less than 11 μM, less than 12 μM, less than 13 μM, less than 14 μM, less than 15 μM, less than 16 μM, less than 17 μM, less than 18 μM, less than 19 μM, less than 20 μM, less than 21 μM, less than 22 μM, less than 23 μM, less than 24 μM, less than 25 μM, less than 26 μM, less than 27 μM, less than 28 μM, less than 29 μM, less than 30 μM, less than 31 μM, less than 32 μM, less than 33 μM, less than 34 μM, less than 35 μM, less than 36 μM, less than 37 μM, less than 38 μM, less than 39 μM, less than 40 μM, less than 41 μM, less than 42 μM, less than 43 μM, less than 44 μM, less than 45 μM, less than 46 μM, less than 47 μM, less than 48 μM, less than 49 μM, less than 50 μM, less than 51 μM, less than 52 μM, less than 53 μM, less than 54 μM, less than 55 μM, less than 56 μM, less than 57 μM, less than 58 μM, less than 59 μM, less than 60 μM, less than 61 μM, less than 62 μM, less than 63 μM, less than 64 μM, less than 65 μM, less than 66 μM, less than 67 μM, less than 68 μM, less than 69 μM, less than 70 μM, less than 71 μM, less than 72 μM, less than 73 μM, less than 74 μM, less than 75 μM, less than 76 μM, less than 77 μM, less than 78 μM, less than 79 μM, less than 80 μM, less than 81 μM, less than 82 μM, less than 83 μM, less than 84 μM, less than 85 μM, less than 86 μM, less than 87 μM, less than 88 μM, less than 89 μM, less than 90 μM, less than 91 μM, less than 92 μM, less than 93 μM, less than 94 μM, less than 95 μM, less than 96 μM, less than 97 μM, less than 98 μM, less than 99 μM, less than 100 μM, less than 200 μM, less than 300 μM, less than 400 μM, less than 500 μM, less than 600 μM, less than 700 μM, less than 800 μM, less than 900 μM, less than 1000 μM, less than 1100 μM, less than 1200 μM, less than 1300 μM, less than 1400 μM, less than 1500 μM, less than 1600 μM, less than 1700 μM, less than 1800 μM, less than 1900 μM, or less than 2000 μM. In some embodiments, the Kₘ is between 0.1 μM and 2000 μM, between 1 μM and 1000 μM, between 1 μM and 100 μM, between 0.1 μM and 1000 μM, between 100 μM and 2000 μM, between 100 μM and 1000 μM, between 1000 μM and 2000 μM, between 500 μM and 1500 μM, between 500 μM and 1500 μM, or between 0.1 μM and 1000 μM.

In some embodiments, the activity of an ACL described herein is measured as the catalytic efficiency (k_{cat}/kₘ). In some embodiments, the catalytic efficiency is measured in units of s⁻¹ mM⁻¹. In some embodiments, the catalytic efficiency is greater than 0.1, greater than 0.2, greater than 0.3, greater than 0.4, greater than 0.5, greater than 0.6, greater than 0.7, greater than 0.8, greater than 0.9, greater than 1, greater than 2, greater than 3, greater than 4, greater than 5, greater than 6, greater than 7, greater than 8, greater than 9, greater than 10, greater than 11, greater than 12, greater than 13, greater than 14, greater than 15, greater than 16, greater than 17, greater than 18, greater than 19, greater than 20, greater than 21, greater than 22, greater than 23, greater than 24, greater than 25, greater than 26, greater than 27, greater than 28, greater than 29, greater than 30, greater than 31, greater than 32, greater than 33, greater than 34, greater than 35, greater than 36, greater than 37, greater than 38, greater than 39, greater than 40, greater than 41, greater than 42, greater than 43, greater than 44, greater than 45, greater than 46, greater than 47, greater than 48, greater than 49, greater than 50, greater than 51, greater than 52, greater than 53, greater than 54, greater than 55, greater than 56, greater than 57, greater than 58, greater than 59, greater than 60, greater than 61, greater than 62, greater than 63, greater than 64, greater than 65, greater than 66, greater than 67, greater than 68, greater than 69, greater than 70, greater than 71, greater than 72, greater than 73, greater than 74, greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, greater than 99, greater than 100, greater than 500, greater than 1000 s⁻¹ mM⁻¹. In some embodiments, the catalytic efficiency (k_{cat}/kₘ) is between 1 and 30 s⁻¹ mM⁻¹, between 5 and 30 s⁻¹ mM⁻¹, between 1 and 10 s⁻¹ mM⁻¹, between 10 and 30 s⁻¹ mM⁻¹, or between 20 and 30 s⁻¹ mM⁻¹.

In some embodiments, an engineered ACL provided herein is a variant of a reference polypeptide, wherein the reference polypeptide has an amino acid sequence of SEQ ID NO: 1, and the engineered ACL has one or more alterations at a position described in TABLE 8 relative to SEQ ID NO: 1. Accordingly, in some embodiments, an engineered ACL provided herein includes one or more amino acid alterations at a residue corresponding to position 4, 7, 8, 9, 10, 12, 13, 15, 16, 19, 20, 22, 30, 31, 35, 36, 39, 40, 43, 44, 47, 48, 49, 50, 51, 52, 53, 54, 55, 60, 61, 64, 66, 67, 69, 72, 73, 74, 76, 78, 80, 81, 82, 83, 86, 87, 88, 89, 90, 91, 94, 95, 96, 97, 98, 99, 100, 101, 102, 103, 104, 105, 106, 107, 109, 111, 112, 113, 114, 115, 116, 117, 119, 121, 126, 127, 128, 129, 130, 131, 136, 137, 138, 139, 140, 144, 149, 150, 152, 153, 159, 162, 163, 164, 165, 166, 167, 170, 172, 182, 189, 191, 194, 195, 196, 197, 198, 199, 200, 201, 202, 205, 208, 209, 211, 212, 213, 214, 215, 217, 220, 221, 222, 223, 224, 225, 226, 227, 228, 229, 230, 231, 232, 233, 234, 235, 238, 239, 240, 241, 242, 243, 244, 246, 247, 248, 249, 250, 251, 252, 253, 254, 255, 256, 257, 258, 259, 260, 261, 262, 264, 265, 266, 267, 268, 269, 270, 271, 272, 274, 275, 276, 277, 278, 279, 280, 281, 282, 286, 287, 288, 289, 290, 291, 294, 295, 296, 297, 298, 299, 300, 304, 306, 309, 310, 311, 314, 315, 316, 317, 318, 319, 320, 321, 323, 324, 326, 327, 328, 329, 330, 331, 334, 335, 336, 338, 339, 341, 342, 348, 351, 352, 353, 354, 355, 356, 357, 359, 360, 362, 363, 364, 365, 366, 367, 368, 369, 370, 371, 372, 373, 374, 375, 376, 377, 378, 379, 380, 381, 383, 384, 386, 387, 388, 389, 390, 391, 394, 398, 402, 406, 407, 410, 411, 412, 413, 414, 415, 416, 418, 419, 420, 424, 425, 426, 430, 431, 432, 433, 436, 442, 443, 444, 445, 446, 447, 455, 459, 461, 462, 464, 465, 469, 470, 471, 472, 474, 475, 476, 477, 478, 479, 480, 481, 484, 486, 489, 490, 491, 493, 494, 496, 503, 506, 507, 508, 509, 513, 518, 519, 521, 523, 524, 525, 527, 528, 529, 539, 540, 542, 546, 551, 553, 554, 555, 557, 559, 560, 562, 564, 565, 566, 583, more than one of the foregoing, or a combination thereof, in SEQ ID NO: 1. In some embodiments, an engineered ACL provided herein includes one or more amino acid alterations at a residue corresponding to position 7, 8, 10, 12, 15, 16, 19, 52, 54, 64, 66, 67, 73, 74, 82, 87, 90, 94, 100, 111, 115, 117, 119, 121, 129, 131, 138, 139, 140, 144, 150, 165, 166, 172, 182, 191, 199, 209, 212, 217, 221, 223, 224, 227, 229, 230, 231, 232, 233, 234, 235, 238, 240, 243, 249, 253, 254, 256, 258, 259, 262, 268, 271, 275, 277, 281, 288, 290, 296, 297, 300, 304, 306, 316, 318, 320, 321, 323, 328, 330, 331, 336, 352, 353, 363, 364, 365, 366, 369, 372, 374, 376, 378, 388, 390, 394, 398, 406, 407, 416, 432, 443, 445, 455, 462, 469, 470, 471, 480, 481, 484, 486, 490, 491, 493, 494, 496, 508, 513, 518, 519, 521, 523, 525, 546, 554, 555, 560, 565, 583, more than one of the foregoing, or a combination thereof, in SEQ ID NO: 1. In some embodiments, an engineered ACL provided herein includes one or more amino acid alterations at a residue corresponding to position 10, 199, 223, 229, 230, 231, 232, 233, 234, 235, 238, 243, 256, 258, 277, 288, 300, 318, 320, 321, 323, 328, 330, 364, 365, 369, 372, 374, 378, 462, 469, 480, 481, 484, 513, 523, 525, 554, 560, 583, more than one of the foregoing, or a combination thereof, in SEQ ID NO: 1. In some embodiments, an engineered ACL provided herein includes one or more amino acid alterations at a residue corresponding to position 223, 232, 233, 234, 238, 258, 288, 318, 320, 321, 323, 364, 513, 523, more than one of the foregoing, or a combination thereof, in SEQ ID NO: 1.

In some embodiments, an engineered ACL provided herein is a variant of a reference polypeptide, wherein the reference polypeptide has an amino acid sequence of SEQ ID NO: 2, and the engineered ACL has one or more alterations at a position described in TABLE 9 relative to SEQ ID NO: 2. Accordingly, in some embodiments, an engineered ACL provided herein includes one or more amino acid alterations at a residue corresponding to position 5, 6, 7, 8, 10, 12, 13, 14, 15, 16, 19, 20, 22, 25, 28, 37, 39, 40, 41, 43, 44, 45, 48, 50, 51, 53, 57, 58, 59, 61, 62, 63, 66, 67, 69, 71, 72, 74, 75, 76, 77, 78, 80, 81, 83, 84, 85, 86, 87, 88, 92, 93, 94, 95, 97, 98, 99, 100, 101, 103, 104, 106, 107, 108, 109, 110, 113, 114, 115, 116, 119, 120, 121, 122, 123, 124, 125, 126, 127, 128, 130, 131, 136, 137, 140, 141, 142, 145, 147, 148, 149, 150, 151, 157, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 216, 218, 219, 220, 221, 222, 224, 225, 226, 227, 228, 230, 231, 232, 233, 234, 235, 236, 237, 238, 239, 240, 241, 242, 243, 244, 245, 246, 247, 251, 252, 253, 254, 255, 256, 257, 259, 260, 262, 263, 264, 265, 266, 267, 268, 269, 270, 271, 272, 273, 274, 275, 277, 278, 279, 281, 282, 283, 284, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 341, 342, 343, 344, 345, 346, 347, 348, 349, 351, 352, 353, 354, 355, 357, 358, 359, 361, 362, 363, 365, 366, 367, 368, 369, 370, 372, 373, 375, 376, 377, 378, 379, 380, 381, 382, 383, 384, 385, 386, 387, 388, 389, 391, 392, 394, 395, 396, 398, 399, 400, 401, 402, 404, 405, 406, 407, 409, 411, 413, 414, 415, 416, 417, 418, 419, 420, 421, 425, 430, 432, 433, 434, 435, 441, 442, 443, 446, 447, 449, 450, 451, 452, 453, 455, 456, 458, 459, 460, 461, 462, 463, 464, 465, 466, 467, 468, 469, 470, 471, 473, 474, 476, 478, 479, 480, 481, 483, 485, 490, 492, 493, 496, 497, 498, 502, 504, 507, 508, 510, 511, 512, 513, 514, 515, 516, 518, 519, 521, 526, 527, 532, 535, 540, 542, 546, 552, 554, 558, 560, 561, 563, 564, 565, 566, 567, 568, 569, 570, 572, 573, 574, 575, 577, 578, 583, 584, 585, 587, 588, 591, 593, 595, 596, 598, 599, 601, more than one of the foregoing, or a combination thereof, in SEQ ID NO: 2. In some embodiments, an engineered ACL provided herein includes one or more amino acid alterations at a residue corresponding to position 5, 6, 7, 8, 10, 12, 13, 14, 15, 16, 19, 20, 22, 25, 28, 37, 39, 40, 41, 43, 45, 50, 51, 53, 57, 58, 59, 61, 62, 63, 67, 69, 71, 72, 74, 75, 76, 77, 78, 80, 81, 83, 84, 85, 86, 87, 88, 92, 93, 94, 95, 97, 98, 99, 100, 101, 103, 104, 106, 108, 109, 110, 113, 116, 119, 120, 121, 123, 124, 125, 126, 127, 128, 130, 131, 136, 137, 140, 141, 142, 145, 147, 148, 149, 150, 151, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 218, 219, 220, 221, 222, 224, 226, 227, 228, 230, 232, 233, 234, 235, 236, 237, 238, 239, 240, 241, 242, 243, 244, 245, 246, 247, 252, 253, 254, 255, 256, 260, 262, 263, 264, 265, 266, 267, 268, 270, 271, 272, 273, 274, 275, 277, 278, 279, 281, 283, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 342, 343, 344, 345, 346, 347, 348, 349, 351, 352, 353, 354, 355, 357, 358, 359, 362, 363, 365, 366, 367, 368, 369, 370, 375, 376, 377, 378, 379, 380, 381, 382, 383, 384, 385, 386, 387, 388, 389, 391, 392, 394, 396, 398, 399, 400, 401, 404, 406, 407, 409, 411, 414, 416, 419, 420, 421, 425, 430, 432, 433, 434, 435, 441, 442, 443, 446, 447, 450, 451, 452, 453, 456, 460, 461, 462, 463, 465, 466, 467, 468, 469, 470, 471, 473, 476, 478, 479, 480, 481, 485, 490, 492, 498, 507, 508, 510, 511, 512, 514, 515, 516, 518, 519, 521, 526, 527, 532, 535, 540, 542, 546, 552, 554, 560, 561, 563, 564, 565, 566, 567, 568, 569, 570, 572, 573, 574, 575, 577, 578, 583, 584, 585, 587, 588, 591, 593, 595, 596, 598, 599, 601, more than one of the foregoing, or a combination thereof, in SEQ ID NO: 2. In some embodiments, an engineered ACL provided herein includes one or more amino acid alterations at a residue corresponding to position 5, 6, 7, 8, 10, 12, 14, 15, 16, 19, 20, 22, 28, 39, 41, 43, 45, 50, 57, 58, 61, 62, 63, 67, 69, 71, 72, 76, 77, 78, 81, 84, 85, 86, 87, 92, 93, 94, 95, 98, 104, 108, 109, 113, 116, 119, 120, 123, 124, 125, 126, 127, 130, 131, 136, 140, 141, 142, 145, 147, 148, 149, 151, 159, 161, 163, 164, 167, 168, 169, 170, 173, 175, 176, 177, 181, 183, 185, 186, 187, 190, 191, 193, 194, 195, 201, 205, 209, 214, 218, 219, 220, 221, 226, 228, 230, 232, 234, 237, 238, 239, 241, 242, 243, 244, 246, 247, 253, 254, 260, 262, 263, 266, 267, 272, 273, 274, 278, 279, 281, 283, 285, 290, 291, 292, 295, 296, 298, 300, 302, 303, 304, 305, 307, 311, 313, 314, 315, 316, 318, 321, 322, 330, 333, 334, 336, 337, 338, 339, 340, 344, 345, 346, 347, 348, 349, 351, 352, 353, 354, 355, 357, 358, 359, 362, 363, 365, 367, 369, 375, 376, 379, 382, 383, 384, 386, 387, 388, 389, 399, 400, 401, 404, 407, 409, 416, 419, 420, 421, 430, 432, 433, 434, 441, 442, 446, 450, 452, 453, 456, 460, 461, 465, 466, 471, 473, 476, 479, 480, 481, 485, 490, 492, 507, 511, 514, 515, 516, 518, 519, 521, 526, 527, 532, 540, 542, 546, 554, 561, 564, 565, 566, 567, 568, 570, 572, 574, 575, 577, 584, 587, 595, 596, 599, more than one ofthe foregoing, or a combination thereof, in SEQ ID NO: 2. In some embodiments, an engineered ACL provided herein includes one or more amino acid alterations at a residue corresponding to position 5, 6, 7, 8, 10, 12, 14, 15, 19, 20, 22, 41, 45, 50, 58, 62, 63, 67, 69, 72, 76, 77, 81, 86, 87, 92, 93, 94, 95, 98, 104, 108, 113, 116, 120, 123, 124, 125, 126, 127, 130, 136, 140, 141, 142, 145, 147, 148, 149, 151, 159, 161, 163, 164, 167, 168, 169, 175, 176, 177, 185, 186, 187, 190, 191, 193, 195, 201, 205, 209, 214, 219, 220, 221, 228, 230, 232, 234, 237, 238, 239, 241, 242, 243, 244, 246, 247, 253, 254, 260, 263, 266, 267, 272, 273, 274, 278, 279, 281, 283, 291, 292, 295, 298, 300, 302, 303, 304, 305, 307, 311, 313, 314, 315, 318, 321, 322, 330, 333, 334, 336, 337, 338, 340, 344, 348, 349, 351, 353, 354, 355, 357, 358, 359, 362, 363, 365, 367, 369, 376, 379, 382, 383, 386, 387, 388, 389, 399, 400, 401, 404, 407, 409, 420, 421, 430, 432, 433, 434, 442, 446, 450, 452, 453, 456, 460, 461, 465, 466, 473, 476, 479, 480, 481, 485, 492, 507, 514, 515, 516, 518, 519, 521, 526, 527, 532, 540, 542, 554, 564, 565, 567, 568, 570, 572, 575, 584, 587, 595, 596, more than one of the foregoing, or a combination thereof, in SEQ ID NO: 2.

In some embodiments, an engineered ACL provided herein includes one or more alterations at a position described in TABLE 8 or 9, wherein the one or more amino acid alterations are conservative amino acid substitutions. In some embodiments, an engineered ACL provided herein includes one or more conservative amino acid substitutions relative to an alteration described in TABLE 8 or 9. As a non-limiting example, a conservative amino acid substitution relative to the M172L substitution in SEQ ID NO: 1 may include substitution of M172 for another non-polar (hydrophobic) amino acid (e.g., Cys (C), Ala (A), Val (V), Ile (I), Pro (P), Phe (F), Met (M), Trp (W), Gly (G), or Tyr (Y)). In some embodiments, an engineered ACL provided herein includes one or more alterations at a position described in TABLE 8 or 9, wherein the one or more amino acid alterations are non-conservative amino acid substitutions. In some embodiments, an engineered ACL provided herein includes a conservative amino acid substitution and/or non-conservative amino acid substitution in 1 to 100 amino acid positions as set forth in TABLE 8 or 9.

In some embodiments, an engineered ACL provided herein can further include a conservative amino acid substitution in from 1 to 50 amino acid positions, or alternatively from 2 to 50 amino acid positions, or alternatively from 3 to 50 amino acid positions, or alternatively from 4 to 50 amino acid positions, or alternatively from 5 to 50 amino acid positions, or alternatively from 6 to 50 amino acid positions, or alternatively from 7 to 50 amino acid positions, or alternatively from 8 to 50 amino acid positions, or alternatively from 9 to 50 amino acid positions, or alternatively from 10 to 50 amino acid positions, or alternatively from 15 to 50 amino acid positions, or alternatively from 20 to 50 amino acid positions, or alternatively from 30 to 50 amino acid positions, or alternatively from 40 to 50 amino acid positions, or alternatively from 45 to 50 amino acid positions, or any integer therein, wherein the positions are other than the variant amino acid positions set forth in TABLE 8 or 9. In some aspects, such a conservative amino acid sequence is a chemically conservative or an evolutionary conservative amino acid substitution. Methods of identifying conservative amino acids are well known to one of skill in the art, any one of which can be used to generate the isolated engineered ACLs described herein.

An engineered ACL provided herein may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 alterations relative to a wild-type ACL (SEQ ID NO: 1 or 2). An engineered ACL provided herein may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, at most 101, at most 102, at most 103, at most 104, at most 105, at most 106, at most 107, at most 108, at most 109, at most 110, at most 111, at most 112, at most 113, at most 114, at most 115, at most 116, at most 117, at most 118, at most 119, at most 120, at most 121, at most 122, at most 123, at most 124, at most 125, at most 126, at most 127, at most 128, at most 129, at most 130, at most 131, at most 132, at most 133, at most 134, at most 135, at most 136, at most 137, at most 138, at most 139, at most 140, at most 141, at most 142, at most 143, at most 144, at most 145, at most 146, at most 147, at most 148, at most 149, at most 150, at most 151, at most 152, at most 153, at most 154, at most 155, at most 156, at most 157, at most 158, at most 159, at most 160, at most 161, at most 162, at most 163, at most 164, at most 165, at most 166, at most 167, at most 168, at most 169, at most 170, at most 171, at most 172, at most 173, at most 174, at most 175, at most 176, at most 177, at most 178, at most 179, at most 180, at most 181, at most 182, at most 183, at most 184, at most 185, at most 186, at most 187, at most 188, at most 189, at most 190, at most 191, at most 192, at most 193, at most 194, at most 195, at most 196, at most 197, at most 198, at most 199, at most 200, at most 201, at most 202, at most 203, at most 204, at most 205, at most 206, at most 207, at most 208, at most 209, at most 210, at most 211, at most 212, at most 213, at most 214, at most 215, at most 216, at most 217, at most 218, at most 219, at most 220, at most 221, at most 222, at most 223, at most 224, at most 225, at most 226, at most 227, at most 228, at most 229, at most 230, at most 231, at most 232, at most 233, at most 234, at most 235, at most 236, at most 237, at most 238, at most 239, at most 240, at most 241, at most 242, at most 243, at most 244, at most 245, at most 246, at most 247, at most 248, at most 249, or at most 250 alterations relative to a wild-type ACL (SEQ ID NO: 1 or 2). The one or more alterations may be located at one or more positions corresponding to the one or more positions described in TABLE 8 or 9. The one or more alterations may be located at one or more positions corresponding to one or more positions in SEQ ID NO: 1 or 2. As used herein, the phrase “a residue corresponding to position X in SEQ ID NO: Y” refers to a residue at a corresponding position following an alignment of two sequences. For example, the residue in SEQ ID NO: 1 corresponding to position 370 in SEQ ID NO: 1 is the residue at position 370 in SEQ ID NO: 1. In some embodiments, a reference sequence is an ACL that is not SEQ ID NO: 1 or 2.

An engineered ACL provided herein can include any combination of the alterations set forth in TABLE 8 or 9. One alteration alone, or in combination, can produce an engineered ACL that retains or improves the activity as described herein relative to a reference polypeptide, for example, the wild-type ACL (SEQ ID NO: 1 or 2). In some embodiments, an engineered ACL provided herein includes at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 alterations as set forth in TABLE 8 or 9, including up to an alteration at all of the positions identified in TABLE 8 or 9. In some embodiments, an engineered ACL provided herein includes at least 2 alterations as set forth in TABLE 8 or 9. In some embodiments, an engineered ACL provided herein includes at least 3 alterations as set forth in TABLE 8 or 9. In some embodiments, an engineered ACL provided herein includes at least 4 alterations as set forth in TABLE 8 or 9. In some embodiments, an engineered ACL provided herein includes at least 5 alterations as set forth in TABLE 8 or 9. In some embodiments, an engineered ACL provided herein includes at least 6 alterations as set forth in TABLE 8 or 9. In some embodiments, an engineered ACL provided herein includes at least 7 alterations as set forth in TABLE 8 or 9. In some embodiments, an engineered ACL provided herein includes at least 8 alterations as set forth in TABLE 8 or 9. In some embodiments, an engineered ACL provided herein includes at least 9 alterations as set forth in TABLE 8 or 9. In some embodiments, an engineered ACL provided herein includes at least 10 alterations as set forth in TABLE 8 or 9. In some embodiments, an engineered ACL provided herein includes at least 11 alterations as set forth in TABLE 8 or 9. In some embodiments, an engineered ACL provided herein includes at least 12 alterations as set forth in TABLE 8 or 9. In some embodiments, an engineered ACL provided herein includes at least 13 alterations as set forth in TABLE 8 or 9. In some embodiments, an engineered ACL provided herein includes at least 14 alterations as set forth in TABLE 8 or 9. In some embodiments, an engineered ACL provided herein includes at least 15 alterations as set forth in TABLE 8 or 9. In some embodiments, an engineered ACL provided herein includes at least 16 alterations as set forth in TABLE 8 or 9. In some embodiments, an engineered ACL provided herein includes at least 17 alterations as set forth in TABLE 8 or 9. In some embodiments, an engineered ACL provided herein includes at least 18 alterations as set forth in TABLE 8 or 9. In some embodiments, an engineered ACL provided herein includes at least 19 alterations as set forth in TABLE 8 or 9. In some embodiments, an engineered ACL provided herein includes at least 20 alterations as set forth in TABLE 8 or 9. In some embodiments, an engineered ACL provided herein includes at least 21 alterations as set forth in TABLE 8 or 9. In some embodiments, an engineered ACL provided herein includes at least 22 alterations as set forth in TABLE 8 or 9. In some embodiments, an engineered ACL provided herein includes at least 23 alterations as set forth in TABLE 8 or 9. In some embodiments, an engineered ACL provided herein includes at least 10 alterations as set forth in TABLE 8 or 9. In some embodiments, an engineered ACL provided herein includes at least 24 alterations as set forth in TABLE 8 or 9. In some embodiments, an engineered ACL provided herein includes at least 25 alterations as set forth in TABLE 8 or 9. In some embodiments, an engineered ACL provided herein includes at least 26 alterations as set forth in TABLE 8 or 9. In some embodiments, an engineered ACL provided herein includes at least 27 alterations as set forth in TABLE 8 or 9. In some embodiments, an engineered ACL provided herein includes at least 28 alterations as set forth in TABLE 8 or 9. In some embodiments, an engineered ACL provided herein includes at least 29 alterations as set forth in TABLE 8 or 9. In some embodiments, an engineered ACL provided herein includes at least 30 alterations as set forth in TABLE 8 or 9.

In certain embodiments, the one or more amino acid alterations of the engineered ACL is an alteration described in TABLE 8. For example, in some embodiments, the one or more amino acid alterations result in an engineered ACL having: M at a residue corresponding to position 4 in SEQ ID NO: 1; D, K, Q, S, T at a residue corresponding to position 7 in SEQ ID NO: 1; I, S at a residue corresponding to position 8 in SEQ ID NO: 1; A, K, S at a residue corresponding to position 9 in SEQ ID NO: 1; F, H, P, V, W, Y at a residue corresponding to position 10 in SEQ ID NO: 1; D, E, H, P at a residue corresponding to position 12 in SEQ ID NO: 1; C, M at a residue corresponding to position 13 in SEQ ID NO: 1; D, I, L, P at a residue corresponding to position 15 in SEQ ID NO: 1; E, H, N at a residue corresponding to position 16 in SEQ ID NO: 1; F, L, P, Q at a residue corresponding to position 19 in SEQ ID NO: 1; W at a residue corresponding to position 20 in SEQ ID NO: 1; S at a residue corresponding to position 22 in SEQ ID NO: 1; D at a residue corresponding to position 30 in SEQ ID NO: 1; A at a residue corresponding to position 31 in SEQ ID NO: 1; G, P at a residue corresponding to position 35 in SEQ ID NO: 1; H at a residue corresponding to position 36 in SEQ ID NO: 1; A at a residue corresponding to position 39 in SEQ ID NO: 1; C, F, L, M at a residue corresponding to position 40 in SEQ ID NO: 1; G at a residue corresponding to position 43 in SEQ ID NO: 1; D, H, S, T at a residue corresponding to position 44 in SEQ ID NO: 1; R, RTYTQKEL (SEQ ID NO: 15), RTYTQREL (SEQ ID NO: 16), RTYT (SEQ ID NO: 17) at a residue corresponding to position 47 in SEQ ID NO: 1; a deletion at a residue corresponding to position 48 in SEQ ID NO: 1; a deletion, Y at a residue corresponding to position 49 in SEQ ID NO: 1; a deletion, T at a residue corresponding to position 50 in SEQ ID NO: 1; a deletion, F, H, L, R, W, Y at a residue corresponding to position 51 in SEQ ID NO: 1; a deletion, K, R at a residue corresponding to position 52 in SEQ ID NO: 1; a deletion at a residue corresponding to position 53 in SEQ ID NO: 1; a deletion, L at a residue corresponding to position 54 in SEQ ID NO: 1; L at a residue corresponding to position 55 in SEQ ID NO: 1; R at a residue corresponding to position 60 in SEQ ID NO: 1; F, L at a residue corresponding to position 61 in SEQ ID NO: 1; A, G at a residue corresponding to position 64 in SEQ ID NO: 1; R at a residue corresponding to position 66 in SEQ ID NO: 1; A at a residue corresponding to position 67 in SEQ ID NO: 1; L at a residue corresponding to position 69 in SEQ ID NO: 1; G, T at a residue corresponding to position 72 in SEQ ID NO: 1; F, G, L, V at a residue corresponding to position 73 in SEQ ID NO: 1; K at a residue corresponding to position 74 in SEQ ID NO: 1; T at a residue corresponding to position 76 in SEQ ID NO: 1; V at a residue corresponding to position 78 in SEQ ID NO: 1; G at a residue corresponding to position 80 in SEQ ID NO: 1; PG at a residue corresponding to position 81 in SEQ ID NO: 1; A, E, G, N, R, T at a residue corresponding to position 82 in SEQ ID NO: 1; a deletion, K, P, R, S at a residue corresponding to position 83 in SEQ ID NO: 1; I at a residue corresponding to position 86 in SEQ ID NO: 1; I, V, VAV at a residue corresponding to position 87 in SEQ ID NO: 1; a deletion, A at a residue corresponding to position 88 in SEQ ID NO: 1; a deletion, I, L, V at a residue corresponding to position 89 in SEQ ID NO: 1; C, L, M, V, W at a residue corresponding to position 90 in SEQ ID NO: 1; A, L, M, S at a residue corresponding to position 91 in SEQ ID NO: 1; C, CIEAA (SEQ ID NO: 18), S, T, TIEAA (SEQ ID NO: 19) at a residue corresponding to position 94 in SEQ ID NO: 1; a deletion at a residue corresponding to position 95 in SEQ ID NO: 1; a deletion, E at a residue corresponding to position 96 in SEQ ID NO: 1; a deletion at a residue corresponding to position 97 in SEQ ID NO: 1; a deletion, A, L at a residue corresponding to position 98 in SEQ ID NO: 1; C, M at a residue corresponding to position 99 in SEQ ID NO: 1; S, M, L at a residue corresponding to position 100 in SEQ ID NO: 1; F, M at a residue corresponding to position 101 in SEQ ID NO: 1; H, F, L at a residue corresponding to position 102 in SEQ ID NO: 1; G at a residue corresponding to position 103 in SEQ ID NO: 1; V, L, T at a residue corresponding to position 104 in SEQ ID NO: 1; V, W, H, A, I at a residue corresponding to position 105 in SEQ ID NO: 1; S, Y, R, K at a residue corresponding to position 106 in SEQ ID NO: 1; A, C, V at a residue corresponding to position 107 in SEQ ID NO: 1; L, V, Q at a residue corresponding to position 109 in SEQ ID NO: 1; V, A, M, L, C, VTPAN (SEQ ID NO: 20) at a residue corresponding to position 111 in SEQ ID NO: 1; T, a deletion at a residue corresponding to position 112 in SEQ ID NO: 1; a deletion, L at a residue corresponding to position 113 in SEQ ID NO: 1; a deletion, A, L, V at a residue corresponding to position 114 in SEQ ID NO: 1; a deletion, N at a residue corresponding to position 115 in SEQ ID NO: 1; A, W, P at a residue corresponding to position 116 in SEQ ID NO: 1; S at a residue corresponding to position 117 in SEQ ID NO: 1; T at a residue corresponding to position 119 in SEQ ID NO: 1; R, D at a residue corresponding to position 121 in SEQ ID NO: 1; H at a residue corresponding to position 126 in SEQ ID NO: 1; M, L, MRHTG (SEQ ID NO: 21), MRITG (SEQ ID NO: 22) at a residue corresponding to position 127 in SEQ ID NO: 1; a deletion at a residue corresponding to position 128 in SEQ ID NO: 1; a deletion, I, H at a residue corresponding to position 129 in SEQ ID NO: 1; a deletion at a residue corresponding to position 130 in SEQ ID NO: 1; a deletion, G, D at a residue corresponding to position 131 in SEQ ID NO: 1; L at a residue corresponding to position 136 in SEQ ID NO: 1; T at a residue corresponding to position 137 in SEQ ID NO: 1; D, S at a residue corresponding to position 138 in SEQ ID NO: 1; E at a residue corresponding to position 139 in SEQ ID NO: 1; E, K, D at a residue corresponding to position 140 in SEQ ID NO: 1; T at a residue corresponding to position 144 in SEQ ID NO: 1; R at a residue corresponding to position 149 in SEQ ID NO: 1; I, L at a residue corresponding to position 150 in SEQ ID NO: 1; A, T, C, I at a residue corresponding to position 152 in SEQ ID NO: 1; G, S at a residue corresponding to position 153 in SEQ ID NO: 1; G at a residue corresponding to position 159 in SEQ ID NO: 1; S, H, R, SIDAF (SEQ ID NO: 23), RIESF (SEQ ID NO: 24), HFDSF (SEQ ID NO: 25), HFDAF (SEQ ID NO: 26), HIDSF (SEQ ID NO: 27) at a residue corresponding to position 162 in SEQ ID NO: 1; F, I, a deletion at a residue corresponding to position 163 in SEQ ID NO: 1; a deletion, D at a residue corresponding to position 164 in SEQ ID NO: 1; a deletion, S at a residue corresponding to position 165 in SEQ ID NO: 1; a deletion, F at a residue corresponding to position 166 in SEQ ID NO: 1; L at a residue corresponding to position 167 in SEQ ID NO: 1; G at a residue corresponding to position 170 in SEQ ID NO: 1; L, A, D at a residue corresponding to position 172 in SEQ ID NO: 1; Q at a residue corresponding to position 182 in SEQ ID NO: 1; E, G at a residue corresponding to position 189 in SEQ ID NO: 1; A at a residue corresponding to position 191 in SEQ ID NO: 1; D at a residue corresponding to position 194 in SEQ ID NO: 1; T, P, M at a residue corresponding to position 195 in SEQ ID NO: 1; T, I at a residue corresponding to position 196 in SEQ ID NO: 1; W, C at a residue corresponding to position 197 in SEQ ID NO: 1; Y, V, S, W, I, M, T at a residue corresponding to position 198 in SEQ ID NO: 1; M, L, W, P, F, C, A at a residue corresponding to position 199 in SEQ ID NO: 1; W at a residue corresponding to position 200 in SEQ ID NO: 1; F, T, N, P, Y, L at a residue corresponding to position 201 in SEQ ID NO: 1; T, H, A, Q, E, L, G at a residue corresponding to position 202 in SEQ ID NO: 1; T at a residue corresponding to position 205 in SEQ ID NO: 1; F at a residue corresponding to position 208 in SEQ ID NO: 1; P at a residue corresponding to position 209 in SEQ ID NO: 1; G at a residue corresponding to position 211 in SEQ ID NO: 1; A, C at a residue corresponding to position 212 in SEQ ID NO: 1; M, V, E at a residue corresponding to position 213 in SEQ ID NO: 1; V at a residue corresponding to position 214 in SEQ ID NO: 1; S at a residue corresponding to position 215 in SEQ ID NO: 1; Q, R, Y, H, K, G at a residue corresponding to position 217 in SEQ ID NO: 1; W, M, V at a residue corresponding to position 220 in SEQ ID NO: 1; T, A at a residue corresponding to position 221 in SEQ ID NO: 1; S, N at a residue corresponding to position 222 in SEQ ID NO: 1; A, N, I, L, H at a residue corresponding to position 223 in SEQ ID NO: 1; T, Q, H, I, N, M at a residue corresponding to position 224 in SEQ ID NO: 1; L, A, F, Q at a residue corresponding to position 225 in SEQ ID NO: 1; Y, C, T, W at a residue corresponding to position 226 in SEQ ID NO: 1; G, E, M, T, A, GKWSQD (SEQ ID NO: 28), GKWSQSY (SEQ ID NO: 29), GKWSQ (SEQ ID NO: 30), GKWSQDLK (SEQ ID NO: 31), GKWSQQLQ (SEQ ID NO: 32) at a residue corresponding to position 227 in SEQ ID NO: 1; a deletion at a residue corresponding to position 228 in SEQ ID NO: 1; a deletion, F, S, L, G, W, I, M, H at a residue corresponding to position 229 in SEQ ID NO: 1; a deletion, R, M, P, T, E, Q, A at a residue corresponding to position 230 in SEQ ID NO: 1; a deletion, Q, P, H, QDLQ (SEQ ID NO: 33) at a residue corresponding to position 231 in SEQ ID NO: 1; a deletion, W, A, Y, N, K, Q, D at a residue corresponding to position 232 in SEQ ID NO: 1; a deletion, W, R, M, Y, P at a residue corresponding to position 233 in SEQ ID NO: 1; a deletion, F, Y, T, K, R, Q, P, G, H, E at a residue corresponding to position 234 in SEQ ID NO: 1; H, S, R, K, M, Q, D, G at a residue corresponding to position 235 in SEQ ID NO: 1; P, N, H, T, D, DISAC (SEQ ID NO: 34), DVS at a residue corresponding to position 238 in SEQ ID NO: 1; V, F, R, a deletion at a residue corresponding to position 239 in SEQ ID NO: 1; a deletion, F, H, T, W, S, L, C, V at a residue corresponding to position 240 in SEQ ID NO: 1; a deletion, C, M, P, I at a residue corresponding to position 241 in SEQ ID NO: 1; a deletion, G, V, C at a residue corresponding to position 242 in SEQ ID NO: 1; L, C, M at a residue corresponding to position 243 in SEQ ID NO: 1; M, V at a residue corresponding to position 244 in SEQ ID NO: 1; W, F, L, Y at a residue corresponding to position 246 in SEQ ID NO: 1; F, C at a residue corresponding to position 247 in SEQ ID NO: 1; S, P at a residue corresponding to position 248 in SEQ ID NO: 1; A, V, M at a residue corresponding to position 249 in SEQ ID NO: 1; M, A, W at a residue corresponding to position 250 in SEQ ID NO: 1; A, H at a residue corresponding to position 251 in SEQ ID NO: 1; I, M, W, Q at a residue corresponding to position 252 in SEQ ID NO: 1; T, Q, N, I, H, V, M at a residue corresponding to position 253 in SEQ ID NO: 1; E, I, T, Y, H, V, L, P, A, M, F, S, G at a residue corresponding to position 254 in SEQ ID NO: 1; M, T, I, N, Y, C, ILLANV (SEQ ID NO: 35), ILLANVHY (SEQ ID NO: 36), ILLLNVHY (SEQ ID NO: 37), ILLLNV (SEQ ID NO: 38), ILLLNVHK (SEQ ID NO: 39), ILLHNVHK (SEQ ID NO: 40), ILLSNVHY (SEQ ID NO: 41) at a residue corresponding to position 255 in SEQ ID NO: 1; I, V, H, C, F, a deletion at a residue corresponding to position 256 in SEQ ID NO: 1; a deletion, L, T, N at a residue corresponding to position 257 in SEQ ID NO: 1; a deletion, S, L, P, M, H, T, A at a residue corresponding to position 258 in SEQ ID NO: 1; a deletion, C, A at a residue corresponding to position 259 in SEQ ID NO: 1; a deletion, F, L, M, V at a residue corresponding to position 260 in SEQ ID NO: 1; a deletion, Y, M, W, A, F, L, S at a residue corresponding to position 261 in SEQ ID NO: 1; a deletion, M, K, Y, R, G, C, L, V, S, N at a residue corresponding to position 262 in SEQ ID NO: 1; G, W, I, S, V at a residue corresponding to position 264 in SEQ ID NO: 1; R, C, H, K at a residue corresponding to position 265 in SEQ ID NO: 1; I, V, Q, C at a residue corresponding to position 266 in SEQ ID NO: 1; Y at a residue corresponding to position 267 in SEQ ID NO: 1; V, M, T, F, IMPR (SEQ ID NO: 42), IMNR (SEQ ID NO: 43) at a residue corresponding to position 268 in SEQ ID NO: 1; M, at a residue corresponding to position 269 in SEQ ID NO: 1; a deletion, P at a residue corresponding to position 270 in SEQ ID NO: 1; a deletion, R, Q at a residue corresponding to position 271 in SEQ ID NO: 1; W at a residue corresponding to position 272 in SEQ ID NO: 1; P, PEEMLRAIQ (SEQ ID NO: 44), PEEMLQAIQ (SEQ ID NO: 45) at a residue corresponding to position 274 in SEQ ID NO: 1; D, A, E, R, K, EEMLQAIQ (SEQ ID NO: 46), EEMLRAIQ (SEQ ID NO: 47), a deletion, KEMLQAIQ (SEQ ID NO: 48) at a residue corresponding to position 275 in SEQ ID NO: 1; a deletion at a residue corresponding to position 276 in SEQ ID NO: 1; a deletion, M, Y, L, F at a residue corresponding to position 277 in SEQ ID NO: 1; a deletion at a residue corresponding to position 278 in SEQ ID NO: 1; a deletion, Q, R, QAIQ (SEQ ID NO: 49), RAIQ (SEQ ID NO: 50) at a residue corresponding to position 279 in SEQ ID NO: 1; a deletion at a residue corresponding to position 280 in SEQ ID NO: 1; a deletion, V, M, L, I at a residue corresponding to position 281 in SEQ ID NO: 1; a deletion, Q at a residue corresponding to position 282 in SEQ ID NO: 1; P, C at a residue corresponding to position 286 in SEQ ID NO: 1; T, TVFFA (SEQ ID NO: 51), TSFFA (SEQ ID NO: 52), TFFFA (SEQ ID NO: 53), TMFFA (SEQ ID NO: 54), TVFHA (SEQ ID NO: 55) at a residue corresponding to position 287 in SEQ ID NO: 1; H, F, M, Y, R, I, V, T, C, a deletion at a residue corresponding to position 288 in SEQ ID NO: 1; a deletion, M, V, A, L, F, I at a residue corresponding to position 289 in SEQ ID NO: 1; a deletion, A, W, H, C, F, V, P, L at a residue corresponding to position 290 in SEQ ID NO: 1; a deletion, A, M, G, V at a residue corresponding to position 291 in SEQ ID NO: 1; A, T at a residue corresponding to position 294 in SEQ ID NO: 1; L, a deletion at a residue corresponding to position 295 in SEQ ID NO: 1; M, L at a residue corresponding to position 296 in SEQ ID NO: 1; M, Q, I, A, L, T at a residue corresponding to position 297 in SEQ ID NO: 1; M, A, F, R, IM at a residue corresponding to position 298 in SEQ ID NO: 1; M, F at a residue corresponding to position 299 in SEQ ID NO: 1; A at a residue corresponding to position 300 in SEQ ID NO: 1; M, R, E at a residue corresponding to position 304 in SEQ ID NO: 1; D at a residue corresponding to position 306 in SEQ ID NO: 1; L at a residue corresponding to position 309 in SEQ ID NO: 1; L, D at a residue corresponding to position 310 in SEQ ID NO: 1; L at a residue corresponding to position 311 in SEQ ID NO: 1; S, SHVRSV (SEQ ID NO: 56), SHVRTV (SEQ ID NO: 57) at a residue corresponding to position 314 in SEQ ID NO: 1; S, a deletion at a residue corresponding to position 315 in SEQ ID NO: 1; a deletion, V, L, I at a residue corresponding to position 316 in SEQ ID NO: 1; a deletion, H, N, Q, T at a residue corresponding to position 317 in SEQ ID NO: 1; a deletion, Y, M, T, S, C, Q, R, H, V, W, I, L at a residue corresponding to position 318 in SEQ ID NO: 1; a deletion, C, F, V, L, M at a residue corresponding to position 319 in SEQ ID NO: 1; F, I, V, Y, T, M, W at a residue corresponding to position 320 in SEQ ID NO: 1; C at a residue corresponding to position 321 in SEQ ID NO: 1; G at a residue corresponding to position 323 in SEQ ID NO: 1; S, M at a residue corresponding to position 324 in SEQ ID NO: 1; V, I, C at a residue corresponding to position 326 in SEQ ID NO: 1; S at a residue corresponding to position 327 in SEQ ID NO: 1; H, V, R, S, RELE (SEQ ID NO: 58) at a residue corresponding to position 328 in SEQ ID NO: 1; E, a deletion, ELE at a residue corresponding to position 329 in SEQ ID NO: 1; a deletion, L, T, I at a residue corresponding to position 330 in SEQ ID NO: 1; a deletion, M, E, C, L, I at a residue corresponding to position 331 in SEQ ID NO: 1; A, W, C, M, I at a residue corresponding to position 334 in SEQ ID NO: 1; Q, R at a residue corresponding to position 335 in SEQ ID NO: 1; K at a residue corresponding to position 336 in SEQ ID NO: 1; M at a residue corresponding to position 338 in SEQ ID NO: 1; S at a residue corresponding to position 339 in SEQ ID NO: 1; H, T at a residue corresponding to position 341 in SEQ ID NO: 1; L, F at a residue corresponding to position 342 in SEQ ID NO: 1; D, H, Y at a residue corresponding to position 348 in SEQ ID NO: 1; V, C, T, F, A, W at a residue corresponding to position 351 in SEQ ID NO: 1; E, K, D at a residue corresponding to position 352 in SEQ ID NO: 1; V, L, M at a residue corresponding to position 353 in SEQ ID NO: 1; C, F, L, Y, A, K, S, I at a residue corresponding to position 354 in SEQ ID NO: 1; E at a residue corresponding to position 355 in SEQ ID NO: 1; A at a residue corresponding to position 356 in SEQ ID NO: 1; W at a residue corresponding to position 357 in SEQ ID NO: 1; M at a residue corresponding to position 359 in SEQ ID NO: 1; S at a residue corresponding to position 360 in SEQ ID NO: 1; C, S at a residue corresponding to position 362 in SEQ ID NO: 1; A, T, G at a residue corresponding to position 363 in SEQ ID NO: 1; C, S at a residue corresponding to position 364 in SEQ ID NO: 1; L, I, M, C at a residue corresponding to position 365 in SEQ ID NO: 1; S, V, T at a residue corresponding to position 366 in SEQ ID NO: 1; A, C, S, H at a residue corresponding to position 367 in SEQ ID NO: 1; H at a residue corresponding to position 368 in SEQ ID NO: 1; H, Y, I, C, L, V, W, S, T, M at a residue corresponding to position 369 in SEQ ID NO: 1; Y, R, T, S, H, G, N, Q at a residue corresponding to position 370 in SEQ ID NO: 1; W at a residue corresponding to position 371 in SEQ ID NO: 1; K, W, Q, H, S, R, N at a residue corresponding to position 372 in SEQ ID NO: 1; S, H, P, Q, E at a residue corresponding to position 373 in SEQ ID NO: 1; D, T, A, R, S, P, W, E, Q, H at a residue corresponding to position 374 in SEQ ID NO: 1; P at a residue corresponding to position 375 in SEQ ID NO: 1; T, K, E, Q, H, D at a residue corresponding to position 376 in SEQ ID NO: 1; Y, R, N at a residue corresponding to position 377 in SEQ ID NO: 1; S, Y, P, V, A at a residue corresponding to position 378 in SEQ ID NO: 1; G at a residue corresponding to position 379 in SEQ ID NO: 1; T at a residue corresponding to position 380 in SEQ ID NO: 1; V, S, I at a residue corresponding to position 381 in SEQ ID NO: 1; Q, H, T, Y, W, P at a residue corresponding to position 383 in SEQ ID NO: 1; I, P, A, M at a residue corresponding to position 384 in SEQ ID NO: 1; S, A at a residue corresponding to position 386 in SEQ ID NO: 1; G, S, H at a residue corresponding to position 387 in SEQ ID NO: 1; T, M, V, L at a residue corresponding to position 388 in SEQ ID NO: 1; R, K, E at a residue corresponding to position 389 in SEQ ID NO: 1; C, L, A at a residue corresponding to position 390 in SEQ ID NO: 1; K at a residue corresponding to position 391 in SEQ ID NO: 1; D at a residue corresponding to position 394 in SEQ ID NO: 1; D, N at a residue corresponding to position 398 in SEQ ID NO: 1; V, L, I at a residue corresponding to position 402 in SEQ ID NO: 1; L, V at a residue corresponding to position 406 in SEQ ID NO: 1; D at a residue corresponding to position 407 in SEQ ID NO: 1; N, Q, NTENAPR (SEQ ID NO: 59), NTENAPD (SEQ ID NO: 60) at a residue corresponding to position 410 in SEQ ID NO: 1; T, TENAPD (SEQ ID NO: 61), TQNAPR (SEQ ID NO: 62), a deletion, TENAPR (SEQ ID NO: 63), TQNAPN (SEQ ID NO: 64) at a residue corresponding to position 411 in SEQ ID NO: 1; a deletion, Q at a residue corresponding to position 412 in SEQ ID NO: 1; a deletion, N, NNPN (SEQ ID NO: 65) at a residue corresponding to position 413 in SEQ ID NO: 1; a deletion, A, S, N at a residue corresponding to position 414 in SEQ ID NO: 1; a deletion, P at a residue corresponding to position 415 in SEQ ID NO: 1; a deletion, D, R, N, E at a residue corresponding to position 416 in SEQ ID NO: 1; P at a residue corresponding to position 418 in SEQ ID NO: 1; G at a residue corresponding to position 419 in SEQ ID NO: 1; E at a residue corresponding to position 420 in SEQ ID NO: 1; M, V at a residue corresponding to position 424 in SEQ ID NO: 1; V, L at a residue corresponding to position 425 in SEQ ID NO: 1; M, L at a residue corresponding to position 426 in SEQ ID NO: 1; T, N, S at a residue corresponding to position 430 in SEQ ID NO: 1; I, L at a residue corresponding to position 431 in SEQ ID NO: 1; T, F at a residue corresponding to position 432 in SEQ ID NO: 1; S, K at a residue corresponding to position 433 in SEQ ID NO: 1; W at a residue corresponding to position 436 in SEQ ID NO: 1; T at a residue corresponding to position 442 in SEQ ID NO: 1; R, K, RASFA (SEQ ID NO: 66) at a residue corresponding to position 443 in SEQ ID NO: 1; a deletion at a residue corresponding to position 444 in SEQ ID NO: 1; a deletion, M, T, S, A at a residue corresponding to position 445 in SEQ ID NO: 1; a deletion at a residue corresponding to position 446 in SEQ ID NO: 1; a deletion, A at a residue corresponding to position 447 in SEQ ID NO: 1; S, G at a residue corresponding to position 455 in SEQ ID NO: 1; G, E, D at a residue corresponding to position 459 in SEQ ID NO: 1; D, G at a residue corresponding to position 461 in SEQ ID NO: 1; R, G at a residue corresponding to position 462 in SEQ ID NO: 1; L, Y at a residue corresponding to position 464 in SEQ ID NO: 1; H at a residue corresponding to position 465 in SEQ ID NO: 1; M at a residue corresponding to position 469 in SEQ ID NO: 1; C, G at a residue corresponding to position 470 in SEQ ID NO: 1; Y, K, M, W, I, R at a residue corresponding to position 471 in SEQ ID NO: 1; I, F, C, Y, L, M at a residue corresponding to position 472 in SEQ ID NO: 1; E, EDGH (SEQ ID NO: 67), EDGY (SEQ ID NO: 68) at a residue corresponding to position 474 in SEQ ID NO: 1; D, a deletion at a residue corresponding to position 475 in SEQ ID NO: 1; a deletion at a residue corresponding to position 476 in SEQ ID NO: 1; a deletion, H, F, N, E, Y at a residue corresponding to position 477 in SEQ ID NO: 1; W, I, M at a residue corresponding to position 478 in SEQ ID NO: 1; T, H, Y, F, W at a residue corresponding to position 479 in SEQ ID NO: 1; V, L at a residue corresponding to position 480 in SEQ ID NO: 1; C, I, T at a residue corresponding to position 481 in SEQ ID NO: 1; L at a residue corresponding to position 484 in SEQ ID NO: 1; D at a residue corresponding to position 486 in SEQ ID NO: 1; I at a residue corresponding to position 489 in SEQ ID NO: 1; R, C, V at a residue corresponding to position 490 in SEQ ID NO: 1; G, R, N at a residue corresponding to position 491 in SEQ ID NO: 1; F, H, Y, L at a residue corresponding to position 493 in SEQ ID NO: 1; N at a residue corresponding to position 494 in SEQ ID NO: 1; F, S, Y at a residue corresponding to position 496 in SEQ ID NO: 1; L, V, I at a residue corresponding to position 503 in SEQ ID NO: 1; R, RHPD (SEQ ID NO: 69) at a residue corresponding to position 506 in SEQ ID NO: 1; a deletion at a residue corresponding to position 507 in SEQ ID NO: 1; a deletion, P at a residue corresponding to position 508 in SEQ ID NO: 1; a deletion, D at a residue corresponding to position 509 in SEQ ID NO: 1; A, V, C at a residue corresponding to position 513 in SEQ ID NO: 1; V at a residue corresponding to position 518 in SEQ ID NO: 1; P at a residue corresponding to position 519 in SEQ ID NO: 1; E, P at a residue corresponding to position 521 in SEQ ID NO: 1; Q, A, R, M, Y, D at a residue corresponding to position 523 in SEQ ID NO: 1; S, A at a residue corresponding to position 524 in SEQ ID NO: 1; S, T, D, G, E at a residue corresponding to position 525 in SEQ ID NO: 1; P, T, V at a residue corresponding to position 527 in SEQ ID NO: 1; A, V at a residue corresponding to position 528 in SEQ ID NO: 1; V, A at a residue corresponding to position 529 in SEQ ID NO: 1; L, V at a residue corresponding to position 539 in SEQ ID NO: 1; T at a residue corresponding to position 540 in SEQ ID NO: 1; E, D at a residue corresponding to position 542 in SEQ ID NO: 1; A, K at a residue corresponding to position 546 in SEQ ID NO: 1; K, R at a residue corresponding to position 551 in SEQ ID NO: 1; S at a residue corresponding to position 553 in SEQ ID NO: 1; S, P, H at a residue corresponding to position 554 in SEQ ID NO: 1; F at a residue corresponding to position 555 in SEQ ID NO: 1; K at a residue corresponding to position 557 in SEQ ID NO: 1; P at a residue corresponding to position 559 in SEQ ID NO: 1; G at a residue corresponding to position 560 in SEQ ID NO: 1; V, a deletion at a residue corresponding to position 562 in SEQ ID NO: 1; F at a residue corresponding to position 564 in SEQ ID NO: 1; V, F, FV at a residue corresponding to position 565 in SEQ ID NO: 1; E, VE at a residue corresponding to position 566 in SEQ ID NO: 1; Q, E at a residue corresponding to position 583 in SEQ ID NO: 1; or any combination thereof. It is understood that when alterations are listed herein for a certain position, the alteration can be in the alternative or in the conjunctive depending on the context. For example, the deletion, Y, M, T, S, C, Q, R, H, V, W, I, L at a residue corresponding to position 318 in SEQ ID NO: 1, means that alterations at a residue corresponding to position 318 in SEQ ID NO: 1 can comprise a deletion or Y or M or T or S or C or Q or R or H or V or W or I or L. In another example, the V, F, FV alterations at a residue corresponding to position 565 in SEQ ID NO: 1, means that alterations at a residue corresponding to position 565 in SEQ ID NO: 1 can comprise V or F or FV, wherein FV is F and V.

In certain embodiments, the one or more amino acid alterations of the engineered ACL is an alteration described in TABLE 9. For example, in some embodiments, the one or more amino acid alterations result in an engineered ACL having: R, E at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; K, D, P, N, R at a residue corresponding to position 10 in SEQ ID NO: 2; A, I, V, M at a residue corresponding to position 12 in SEQ ID NO: 2; C at a residue corresponding to position 13 in SEQ ID NO: 2; P, A, Q at a residue corresponding to position 14 in SEQ ID NO: 2; E, L, S, R at a residue corresponding to position 15 in SEQ ID NO: 2; C, L, I at a residue corresponding to position 16 in SEQ ID NO: 2; D, R at a residue corresponding to position 19 in SEQ ID NO: 2; H, K, R, D at a residue corresponding to position 20 in SEQ ID NO: 2; D, E at a residue corresponding to position 22 in SEQ ID NO: 2; N at a residue corresponding to position 25 in SEQ ID NO: 2; M at a residue corresponding to position 28 in SEQ ID NO: 2; C at a residue corresponding to position 37 in SEQ ID NO: 2; R at a residue corresponding to position 39 in SEQ ID NO: 2; T at a residue corresponding to position 40 in SEQ ID NO: 2; M, L, V, I at a residue corresponding to position 41 in SEQ ID NO: 2; S at a residue corresponding to position 43 in SEQ ID NO: 2; M at a residue corresponding to position 44 in SEQ ID NO: 2; I, V at a residue corresponding to position 45 in SEQ ID NO: 2; P at a residue corresponding to position 48 in SEQ ID NO: 2; I, F, R, V, L at a residue corresponding to position 50 in SEQ ID NO: 2; R at a residue corresponding to position 51 in SEQ ID NO: 2; L at a residue corresponding to position 53 in SEQ ID NO: 2; F at a residue corresponding to position 57 in SEQ ID NO: 2; V, L, M, F at a residue corresponding to position 58 in SEQ ID NO: 2; V at a residue corresponding to position 59 in SEQ ID NO: 2; P, E at a residue corresponding to position 61 in SEQ ID NO: 2; D at a residue corresponding to position 62 in SEQ ID NO: 2; D at a residue corresponding to position 63 in SEQ ID NO: 2; G at a residue corresponding to position 66 in SEQ ID NO: 2; K at a residue corresponding to position 67 in SEQ ID NO: 2; F, Y, L at a residue corresponding to position 69 in SEQ ID NO: 2; F, W at a residue corresponding to position 71 in SEQ ID NO: 2; A, R at a residue corresponding to position 72 in SEQ ID NO: 2; F at a residue corresponding to position 74 in SEQ ID NO: 2; D at a residue corresponding to position 75 in SEQ ID NO: 2; T, D, A, R at a residue corresponding to position 76 in SEQ ID NO: 2; R at a residue corresponding to position 77 in SEQ ID NO: 2; A at a residue corresponding to position 78 in SEQ ID NO: 2; K at a residue corresponding to position 80 in SEQ ID NO: 2; C at a residue corresponding to position 81 in SEQ ID NO: 2; S, H, Q, N at a residue corresponding to position 83 in SEQ ID NO: 2; Y, H at a residue corresponding to position 84 in SEQ ID NO: 2; F at a residue corresponding to position 85 in SEQ ID NO: 2; A, Q at a residue corresponding to position 86 in SEQ ID NO: 2; R at a residue corresponding to position 87 in SEQ ID NO: 2; L at a residue corresponding to position 88 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; A at a residue corresponding to position 97 in SEQ ID NO: 2; M at a residue corresponding to position 98 in SEQ ID NO: 2; L at a residue corresponding to position 99 in SEQ ID NO: 2; C at a residue corresponding to position 100 in SEQ ID NO: 2; P at a residue corresponding to position 101 in SEQ ID NO: 2; R at a residue corresponding to position 103 in SEQ ID NO: 2; L, W, A at a residue corresponding to position 104 in SEQ ID NO: 2; W, F at a residue corresponding to position 106 in SEQ ID NO: 2; V, P, I, L, W, F at a residue corresponding to position 107 in SEQ ID NO: 2; V at a residue corresponding to position 108 in SEQ ID NO: 2; V at a residue corresponding to position 109 in SEQ ID NO: 2; M, F, Y, A, D at a residue corresponding to position 110 in SEQ ID NO: 2; T, C, V at a residue corresponding to position 113 in SEQ ID NO: 2; M, W, L, G, A at a residue corresponding to position 114 in SEQ ID NO: 2; A at a residue corresponding to position 115 in SEQ ID NO: 2; I at a residue corresponding to position 116 in SEQ ID NO: 2; V, T, I at a residue corresponding to position 119 in SEQ ID NO: 2; M, F, V, A, L, W, Y at a residue corresponding to position 120 in SEQ ID NO: 2; T at a residue corresponding to position 121 in SEQ ID NO: 2; A at a residue corresponding to position 122 in SEQ ID NO: 2; M, V, I at a residue corresponding to position 123 in SEQ ID NO: 2; Y, S, N at a residue corresponding to position 124 in SEQ ID NO: 2; P, T, W at a residue corresponding to position 125 in SEQ ID NO: 2; M, N, R, A at a residue corresponding to position 126 in SEQ ID NO: 2; F, W, L at a residue corresponding to position 127 in SEQ ID NO: 2; R at a residue corresponding to position 128 in SEQ ID NO: 2; M at a residue corresponding to position 130 in SEQ ID NO: 2; E at a residue corresponding to position 131 in SEQ ID NO: 2; E, Q, K at a residue corresponding to position 136 in SEQ ID NO: 2; M at a residue corresponding to position 137 in SEQ ID NO: 2; M, C, I at a residue corresponding to position 140 in SEQ ID NO: 2; M at a residue corresponding to position 141 in SEQ ID NO: 2; N, Q at a residue corresponding to position 142 in SEQ ID NO: 2; G, R, D at a residue corresponding to position 145 in SEQ ID NO: 2; S at a residue corresponding to position 147 in SEQ ID NO: 2; L, V, A at a residue corresponding to position 148 in SEQ ID NO: 2; V, L at a residue corresponding to position 149 in SEQ ID NO: 2; V at a residue corresponding to position 150 in SEQ ID NO: 2; C at a residue corresponding to position 151 in SEQ ID NO: 2; L at a residue corresponding to position 157 in SEQ ID NO: 2; R, E at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 165 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, V, L at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 189 in SEQ ID NO: 2; V at a residue corresponding to position 190 in SEQ ID NO: 2; A, E at a residue corresponding to position 191 in SEQ ID NO: 2; T at a residue corresponding to position 192 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; P at a residue corresponding to position 196 in SEQ ID NO: 2; A, P, L at a residue corresponding to position 201 in SEQ ID NO: 2; T at a residue corresponding to position 205 in SEQ ID NO: 2; D at a residue corresponding to position 208 in SEQ ID NO: 2; I, T, P, V at a residue corresponding to position 209 in SEQ ID NO: 2; L, T, C, M, V, A at a residue corresponding to position 211 in SEQ ID NO: 2; L, M at a residue corresponding to position 212 in SEQ ID NO: 2; I, C, V, Q, M at a residue corresponding to position 213 in SEQ ID NO: 2; Y at a residue corresponding to position 214 in SEQ ID NO: 2; S at a residue corresponding to position 215 in SEQ ID NO: 2; G at a residue corresponding to position 216 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; T, M, L, R, K, H at a residue corresponding to position 221 in SEQ ID NO: 2; S at a residue corresponding to position 222 in SEQ ID NO: 2; A at a residue corresponding to position 224 in SEQ ID NO: 2; A at a residue corresponding to position 225 in SEQ ID NO: 2; M, I, V at a residue corresponding to position 226 in SEQ ID NO: 2; L, D, H, I at a residue corresponding to position 227 in SEQ ID NO: 2; S at a residue corresponding to position 228 in SEQ ID NO: 2; H, K at a residue corresponding to position 230 in SEQ ID NO: 2; N at a residue corresponding to position 231 in SEQ ID NO: 2; L, I, V at a residue corresponding to position 232 in SEQ ID NO: 2; L, V, M, W, A, I at a residue corresponding to position 233 in SEQ ID NO: 2; S, W at a residue corresponding to position 234 in SEQ ID NO: 2; Q, S at a residue corresponding to position 235 in SEQ ID NO: 2; A, C, I, M, V at a residue corresponding to position 236 in SEQ ID NO: 2; A, M, Q, R, E at a residue corresponding to position 237 in SEQ ID NO: 2; M, S, W, N, H at a residue corresponding to position 238 in SEQ ID NO: 2; F, M, V, C, L at a residue corresponding to position 239 in SEQ ID NO: 2; A, R at a residue corresponding to position 240 in SEQ ID NO: 2; A, H, E, Q at a residue corresponding to position 241 in SEQ ID NO: 2; V, C, T, W at a residue corresponding to position 242 in SEQ ID NO: 2; I, F, M, W, V at a residue corresponding to position 243 in SEQ ID NO: 2; E, P, Q, H, G at a residue corresponding to position 244 in SEQ ID NO: 2; M, L, H, W, Y at a residue corresponding to position 245 in SEQ ID NO: 2; I, T, M at a residue corresponding to position 246 in SEQ ID NO: 2; R, P, E at a residue corresponding to position 247 in SEQ ID NO: 2; E at a residue corresponding to position 251 in SEQ ID NO: 2; H, V at a residue corresponding to position 252 in SEQ ID NO: 2; Y, M, F, L, V at a residue corresponding to position 253 in SEQ ID NO: 2; I, F, V, M at a residue corresponding to position 254 in SEQ ID NO: 2; A, T, N, L, I, G, H, C, V, S at a residue corresponding to position 255 in SEQ ID NO: 2; F, V, I, C, Y at a residue corresponding to position 256 in SEQ ID NO: 2; M at a residue corresponding to position 257 in SEQ ID NO: 2; L, F at a residue corresponding to position 259 in SEQ ID NO: 2; H, S, Y, F at a residue corresponding to position 260 in SEQ ID NO: 2; C, T, A, I, M at a residue corresponding to position 262 in SEQ ID NO: 2; A, Y, H at a residue corresponding to position 263 in SEQ ID NO: 2; A at a residue corresponding to position 264 in SEQ ID NO: 2; M, L, W, F, C at a residue corresponding to position 265 in SEQ ID NO: 2; N, T, M, V at a residue corresponding to position 266 in SEQ ID NO: 2; I, G, M, W, F, L, V, C, A at a residue corresponding to position 267 in SEQ ID NO: 2; M, A, C, G, V at a residue corresponding to position 268 in SEQ ID NO: 2; L, M, I, V at a residue corresponding to position 269 in SEQ ID NO: 2; M, H, Y, T at a residue corresponding to position 270 in SEQ ID NO: 2; A, P, C, T, L at a residue corresponding to position 271 in SEQ ID NO: 2; C, V, P, A, T at a residue corresponding to position 272 in SEQ ID NO: 2; M, V, F, I at a residue corresponding to position 273 in SEQ ID NO: 2; Y, R, V, M, C, H, W at a residue corresponding to position 274 in SEQ ID NO: 2; G, C, S, H at a residue corresponding to position 275 in SEQ ID NO: 2; A, C at a residue corresponding to position 277 in SEQ ID NO: 2; C, T, A, H at a residue corresponding to position 278 in SEQ ID NO: 2; V, M, I at a residue corresponding to position 279 in SEQ ID NO: 2; F, L, V, M at a residue corresponding to position 281 in SEQ ID NO: 2; P, M, E, A, H at a residue corresponding to position 282 in SEQ ID NO: 2; E, D, P, R at a residue corresponding to position 283 in SEQ ID NO: 2; F, R, W at a residue corresponding to position 284 in SEQ ID NO: 2; R, F, W, D at a residue corresponding to position 285 in SEQ ID NO: 2; D, F at a residue corresponding to position 286 in SEQ ID NO: 2; P, A, V at a residue corresponding to position 287 in SEQ ID NO: 2; A at a residue corresponding to position 290 in SEQ ID NO: 2; M, V, L, P, I at a residue corresponding to position 291 in SEQ ID NO: 2; H at a residue corresponding to position 292 in SEQ ID NO: 2; I, L, A at a residue corresponding to position 294 in SEQ ID NO: 2; W, M, V, I at a residue corresponding to position 295 in SEQ ID NO: 2; D, Q at a residue corresponding to position 296 in SEQ ID NO: 2; M, V, L at a residue corresponding to position 298 in SEQ ID NO: 2; A at a residue corresponding to position 299 in SEQ ID NO: 2; Q at a residue corresponding to position 300 in SEQ ID NO: 2; Q at a residue corresponding to position 302 in SEQ ID NO: 2; V, I, A, P, C at a residue corresponding to position 303 in SEQ ID NO: 2; S at a residue corresponding to position 304 in SEQ ID NO: 2; W, Y, V, I, F, H, M at a residue corresponding to position 305 in SEQ ID NO: 2; V, Y, I, M, L, F at a residue corresponding to position 306 in SEQ ID NO: 2; P, W, Y, H, M at a residue corresponding to position 307 in SEQ ID NO: 2; T, A, S, L, G, V, M at a residue corresponding to position 308 in SEQ ID NO: 2; A, R, P, T at a residue corresponding to position 311 in SEQ ID NO: 2; I, M, V, F at a residue corresponding to position 312 in SEQ ID NO: 2; L, F, W at a residue corresponding to position 313 in SEQ ID NO: 2; Q, E, H at a residue corresponding to position 314 in SEQ ID NO: 2; R, A, K, L at a residue corresponding to position 315 in SEQ ID NO: 2; W, M, I, F at a residue corresponding to position 316 in SEQ ID NO: 2; H, D at a residue corresponding to position 318 in SEQ ID NO: 2; R, Q at a residue corresponding to position 321 in SEQ ID NO: 2; Q, K, A, H at a residue corresponding to position 322 in SEQ ID NO: 2; L, F at a residue corresponding to position 330 in SEQ ID NO: 2; G at a residue corresponding to position 331 in SEQ ID NO: 2; S at a residue corresponding to position 333 in SEQ ID NO: 2; I, M, V at a residue corresponding to position 334 in SEQ ID NO: 2; R, K at a residue corresponding to position 335 in SEQ ID NO: 2; T, I, M, Y, V, F, W at a residue corresponding to position 336 in SEQ ID NO: 2; L, M, F, A, V at a residue corresponding to position 337 in SEQ ID NO: 2; W, L, M, V at a residue corresponding to position 338 in SEQ ID NO: 2; F, T, S, C at a residue corresponding to position 339 in SEQ ID NO: 2; G at a residue corresponding to position 340 in SEQ ID NO: 2; A, S at a residue corresponding to position 341 in SEQ ID NO: 2; S at a residue corresponding to position 342 in SEQ ID NO: 2; P, A at a residue corresponding to position 343 in SEQ ID NO: 2; M, I, V, C at a residue corresponding to position 344 in SEQ ID NO: 2; S at a residue corresponding to position 345 in SEQ ID NO: 2; V, A, P, E at a residue corresponding to position 346 in SEQ ID NO: 2; E at a residue corresponding to position 347 in SEQ ID NO: 2; T, I, L at a residue corresponding to position 348 in SEQ ID NO: 2; M, F, L, A, I, Y at a residue corresponding to position 349 in SEQ ID NO: 2; F, R, Q, W at a residue corresponding to position 351 in SEQ ID NO: 2; I, M, Y at a residue corresponding to position 352 in SEQ ID NO: 2; R at a residue corresponding to position 353 in SEQ ID NO: 2; K at a residue corresponding to position 354 in SEQ ID NO: 2; A, R at a residue corresponding to position 355 in SEQ ID NO: 2; W, G, V, L, P, I, F, A at a residue corresponding to position 357 in SEQ ID NO: 2; Q at a residue corresponding to position 358 in SEQ ID NO: 2; H, Q at a residue corresponding to position 359 in SEQ ID NO: 2; F at a residue corresponding to position 361 in SEQ ID NO: 2; H, L, C at a residue corresponding to position 362 in SEQ ID NO: 2; D, V, I, G at a residue corresponding to position 363 in SEQ ID NO: 2; M, V, I at a residue corresponding to position 365 in SEQ ID NO: 2; I, V, L, F at a residue corresponding to position 366 in SEQ ID NO: 2; T, C, M, I, V, L at a residue corresponding to position 367 in SEQ ID NO: 2; N, Q, E, T at a residue corresponding to position 368 in SEQ ID NO: 2; G, A at a residue corresponding to position 369 in SEQ ID NO: 2; Y, F at a residue corresponding to position 370 in SEQ ID NO: 2; L, Q at a residue corresponding to position 372 in SEQ ID NO: 2; S at a residue corresponding to position 373 in SEQ ID NO: 2; A, C at a residue corresponding to position 375 in SEQ ID NO: 2; T, S, C, G at a residue corresponding to position 376 in SEQ ID NO: 2; G, M at a residue corresponding to position 377 in SEQ ID NO: 2; C, T, V at a residue corresponding to position 378 in SEQ ID NO: 2; C, V, I, L, A at a residue corresponding to position 379 in SEQ ID NO: 2; F, C, H, S, M, T, V at a residue corresponding to position 380 in SEQ ID NO: 2; N, F, L, C, V at a residue corresponding to position 381 in SEQ ID NO: 2; L, N, C, T, P at a residue corresponding to position 382 in SEQ ID NO: 2; D, N, R, P, Y at a residue corresponding to position 383 in SEQ ID NO: 2; P, N, D, W at a residue corresponding to position 384 in SEQ ID NO: 2; G, D at a residue corresponding to position 385 in SEQ ID NO: 2; D, V, L, T at a residue corresponding to position 386 in SEQ ID NO: 2; D, R, N, H, E at a residue corresponding to position 387 in SEQ ID NO: 2; K, H at a residue corresponding to position 388 in SEQ ID NO: 2; A, P, L at a residue corresponding to position 389 in SEQ ID NO: 2; S, T at a residue corresponding to position 391 in SEQ ID NO: 2; V, C at a residue corresponding to position 392 in SEQ ID NO: 2; R, K, I, Q, L at a residue corresponding to position 394 in SEQ ID NO: 2; A at a residue corresponding to position 395 in SEQ ID NO: 2; F, I, M, V at a residue corresponding to position 396 in SEQ ID NO: 2; D, H, N at a residue corresponding to position 398 in SEQ ID NO: 2; M, V, T, C, I at a residue corresponding to position 399 in SEQ ID NO: 2; D, Q at a residue corresponding to position 400 in SEQ ID NO: 2; M, I, C, L, A at a residue corresponding to position 401 in SEQ ID NO: 2; R at a residue corresponding to position 402 in SEQ ID NO: 2; V at a residue corresponding to position 404 in SEQ ID NO: 2; D at a residue corresponding to position 405 in SEQ ID NO: 2; E at a residue corresponding to position 406 in SEQ ID NO: 2; P at a residue corresponding to position 407 in SEQ ID NO: 2; Q, E, G at a residue corresponding to position 409 in SEQ ID NO: 2; D, S, V, G, N at a residue corresponding to position 411 in SEQ ID NO: 2; I, V at a residue corresponding to position 413 in SEQ ID NO: 2; W, L, V, C, Q at a residue corresponding to position 414 in SEQ ID NO: 2; I at a residue corresponding to position 415 in SEQ ID NO: 2; R at a residue corresponding to position 416 in SEQ ID NO: 2; S at a residue corresponding to position 417 in SEQ ID NO: 2; D at a residue corresponding to position 418 in SEQ ID NO: 2; H, S, Q, M at a residue corresponding to position 419 in SEQ ID NO: 2; V at a residue corresponding to position 420 in SEQ ID NO: 2; T, F at a residue corresponding to position 421 in SEQ ID NO: 2; H, L at a residue corresponding to position 425 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; T, V at a residue corresponding to position 434 in SEQ ID NO: 2; I, M, L at a residue corresponding to position 435 in SEQ ID NO: 2; F, M, Y at a residue corresponding to position 441 in SEQ ID NO: 2; K, H, Y, R, C at a residue corresponding to position 442 in SEQ ID NO: 2; S at a residue corresponding to position 443 in SEQ ID NO: 2; M, I, L, V at a residue corresponding to position 446 in SEQ ID NO: 2; A, G at a residue corresponding to position 447 in SEQ ID NO: 2; I at a residue corresponding to position 449 in SEQ ID NO: 2; D, M at a residue corresponding to position 450 in SEQ ID NO: 2; E at a residue corresponding to position 451 in SEQ ID NO: 2; P at a residue corresponding to position 452 in SEQ ID NO: 2; G at a residue corresponding to position 453 in SEQ ID NO: 2; E, G, R at a residue corresponding to position 455 in SEQ ID NO: 2; D, E, Q, N at a residue corresponding to position 456 in SEQ ID NO: 2; N, F, Y, W at a residue corresponding to position 458 in SEQ ID NO: 2; Y at a residue corresponding to position 459 in SEQ ID NO: 2; Y, F, I, R, W at a residue corresponding to position 460 in SEQ ID NO: 2; I, L, Y at a residue corresponding to position 461 in SEQ ID NO: 2; I, T, V at a residue corresponding to position 462 in SEQ ID NO: 2; D at a residue corresponding to position 463 in SEQ ID NO: 2; D at a residue corresponding to position 464 in SEQ ID NO: 2; L at a residue corresponding to position 465 in SEQ ID NO: 2; D, K, A at a residue corresponding to position 466 in SEQ ID NO: 2; K at a residue corresponding to position 467 in SEQ ID NO: 2; M at a residue corresponding to position 468 in SEQ ID NO: 2; L at a residue corresponding to position 469 in SEQ ID NO: 2; V at a residue corresponding to position 470 in SEQ ID NO: 2; T, V at a residue corresponding to position 471 in SEQ ID NO: 2; S, N at a residue corresponding to position 473 in SEQ ID NO: 2; E at a residue corresponding to position 474 in SEQ ID NO: 2; N, Y, L, F, V, I at a residue corresponding to position 476 in SEQ ID NO: 2; T, L at a residue corresponding to position 478 in SEQ ID NO: 2; P, V at a residue corresponding to position 479 in SEQ ID NO: 2; L, I, S, P, A, V at a residue corresponding to position 480 in SEQ ID NO: 2; P, E at a residue corresponding to position 481 in SEQ ID NO: 2; E at a residue corresponding to position 483 in SEQ ID NO: 2; A, L, E, R at a residue corresponding to position 485 in SEQ ID NO: 2; C at a residue corresponding to position 490 in SEQ ID NO: 2; P at a residue corresponding to position 492 in SEQ ID NO: 2; F at a residue corresponding to position 493 in SEQ ID NO: 2; H at a residue corresponding to position 496 in SEQ ID NO: 2; A at a residue corresponding to position 497 in SEQ ID NO: 2; C at a residue corresponding to position 498 in SEQ ID NO: 2; P at a residue corresponding to position 502 in SEQ ID NO: 2; H at a residue corresponding to position 504 in SEQ ID NO: 2; Y, V, W at a residue corresponding to position 507 in SEQ ID NO: 2; V at a residue corresponding to position 508 in SEQ ID NO: 2; A at a residue corresponding to position 510 in SEQ ID NO: 2; V, L at a residue corresponding to position 511 in SEQ ID NO: 2; I at a residue corresponding to position 512 in SEQ ID NO: 2; V at a residue corresponding to position 513 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; L, M at a residue corresponding to position 526 in SEQ ID NO: 2; L at a residue corresponding to position 527 in SEQ ID NO: 2; R, H at a residue corresponding to position 532 in SEQ ID NO: 2; L at a residue corresponding to position 535 in SEQ ID NO: 2; Q at a residue corresponding to position 540 in SEQ ID NO: 2; Y, W at a residue corresponding to position 542 in SEQ ID NO: 2; N at a residue corresponding to position 546 in SEQ ID NO: 2; Q at a residue corresponding to position 552 in SEQ ID NO: 2; E at a residue corresponding to position 554 in SEQ ID NO: 2; F at a residue corresponding to position 558 in SEQ ID NO: 2; D at a residue corresponding to position 560 in SEQ ID NO: 2; D at a residue corresponding to position 561 in SEQ ID NO: 2; F, L, W at a residue corresponding to position 563 in SEQ ID NO: 2; D, K, S, L, T, R at a residue corresponding to position 564 in SEQ ID NO: 2; M, I, V, P, T at a residue corresponding to position 565 in SEQ ID NO: 2; A, E, D, P at a residue corresponding to position 566 in SEQ ID NO: 2; K, Q, T, N, S, A at a residue corresponding to position 567 in SEQ ID NO: 2; G, N, D at a residue corresponding to position 568 in SEQ ID NO: 2; M, K at a residue corresponding to position 569 in SEQ ID NO: 2; V, L at a residue corresponding to position 570 in SEQ ID NO: 2; R, P at a residue corresponding to position 572 in SEQ ID NO: 2; T, S at a residue corresponding to position 573 in SEQ ID NO: 2; M, L at a residue corresponding to position 574 in SEQ ID NO: 2; A, Y, K, T at a residue corresponding to position 575 in SEQ ID NO: 2; Y, A, L at a residue corresponding to position 577 in SEQ ID NO: 2; Q, A at a residue corresponding to position 578 in SEQ ID NO: 2; D at a residue corresponding to position 583 in SEQ ID NO: 2; N at a residue corresponding to position 584 in SEQ ID NO: 2; H, L at a residue corresponding to position 585 in SEQ ID NO: 2; A at a residue corresponding to position 587 in SEQ ID NO: 2; Q at a residue corresponding to position 588 in SEQ ID NO: 2; Q, E at a residue corresponding to position 591 in SEQ ID NO: 2; A at a residue corresponding to position 593 in SEQ ID NO: 2; E at a residue corresponding to position 595 in SEQ ID NO: 2; K at a residue corresponding to position 596 in SEQ ID NO: 2; D at a residue corresponding to position 598 in SEQ ID NO: 2; S at a residue corresponding to position 599 in SEQ ID NO: 2; L at a residue corresponding to position 601 in SEQ ID NO: 2; or any combination thereof. It is understood that when alterations are listed herein for a certain position, the alteration can be in the alternative or in the conjunctive depending on the context. For example, the F, L, Y at a residue corresponding to position 69 in SEQ ID NO: 2, means that alterations at a residue corresponding to position 69 in SEQ ID NO: 2 can comprise F or L or Y.

In some embodiments, the one or more amino acid alterations of the engineered ACL is an alteration described in TABLE 8, which results in the engineered ACL having greater or similar activity on HHA, as compared to the WT, when the engineered ACL is expressed in an organism having a pathway for production of HHA and assayed under conditions as described in Example 4. For example, in some embodiments, the one or more amino acid alterations result in an engineered ACL having: K, S at a residue corresponding to position 7 in SEQ ID NO: 1; S at a residue corresponding to position 8 in SEQ ID NO: 1; W, Y, F at a residue corresponding to position 10 in SEQ ID NO: 1; E, P, D at a residue corresponding to position 12 in SEQ ID NO: 1; I at a residue corresponding to position 15 in SEQ ID NO: 1; E at a residue corresponding to position 16 in SEQ ID NO: 1; F at a residue corresponding to position 19 in SEQ ID NO: 1; R, K at a residue corresponding to position 52 in SEQ ID NO: 1; L at a residue corresponding to position 54 in SEQ ID NO: 1; A at a residue corresponding to position 64 in SEQ ID NO: 1; R at a residue corresponding to position 66 in SEQ ID NO: 1; A at a residue corresponding to position 67 in SEQ ID NO: 1; L at a residue corresponding to position 73 in SEQ ID NO: 1; K at a residue corresponding to position 74 in SEQ ID NO: 1; T at a residue corresponding to position 82 in SEQ ID NO: 1; I at a residue corresponding to position 87 in SEQ ID NO: 1; L, M at a residue corresponding to position 90 in SEQ ID NO: 1; T at a residue corresponding to position 94 in SEQ ID NO: 1; L at a residue corresponding to position 100 in SEQ ID NO: 1; V at a residue corresponding to position 111 in SEQ ID NO: 1; N at a residue corresponding to position 115 in SEQ ID NO: 1; S at a residue corresponding to position 117 in SEQ ID NO: 1; T at a residue corresponding to position 119 in SEQ ID NO: 1; D at a residue corresponding to position 121 in SEQ ID NO: 1; I at a residue corresponding to position 129 in SEQ ID NO: 1; G at a residue corresponding to position 131 in SEQ ID NO: 1; D, S at a residue corresponding to position 138 in SEQ ID NO: 1; E at a residue corresponding to position 139 in SEQ ID NO: 1; K, E, D at a residue corresponding to position 140 in SEQ ID NO: 1; T at a residue corresponding to position 144 in SEQ ID NO: 1; L, I at a residue corresponding to position 150 in SEQ ID NO: 1; S at a residue corresponding to position 165 in SEQ ID NO: 1; F at a residue corresponding to position 166 in SEQ ID NO: 1; L, A, D at a residue corresponding to position 172 in SEQ ID NO: 1; Q at a residue corresponding to position 182 in SEQ ID NO: 1; A at a residue corresponding to position 191 in SEQ ID NO: 1; A at a residue corresponding to position 199 in SEQ ID NO: 1; P at a residue corresponding to position 209 in SEQ ID NO: 1; C at a residue corresponding to position 212 in SEQ ID NO: 1; R at a residue corresponding to position 217 in SEQ ID NO: 1; T, A at a residue corresponding to position 221 in SEQ ID NO: 1; N, I, A, L, H at a residue corresponding to position 223 in SEQ ID NO: 1; Q at a residue corresponding to position 224 in SEQ ID NO: 1; E, T, A at a residue corresponding to position 227 in SEQ ID NO: 1; I, L, M at a residue corresponding to position 229 in SEQ ID NO: 1; Q, A at a residue corresponding to position 230 in SEQ ID NO: 1; H, Q, P at a residue corresponding to position 231 in SEQ ID NO: 1; Y, K, Q, N at a residue corresponding to position 232 in SEQ ID NO: 1; W, Y at a residue corresponding to position 233 in SEQ ID NO: 1; Y, F, H at a residue corresponding to position 234 in SEQ ID NO: 1; S at a residue corresponding to position 235 in SEQ ID NO: 1; D, H, T at a residue corresponding to position 238 in SEQ ID NO: 1; T, H, C, S at a residue corresponding to position 240 in SEQ ID NO: 1; C, M at a residue corresponding to position 243 in SEQ ID NO: 1; M, V at a residue corresponding to position 249 in SEQ ID NO: 1; H, M, Q at a residue corresponding to position 253 in SEQ ID NO: 1; M at a residue corresponding to position 254 in SEQ ID NO: 1; I, V at a residue corresponding to position 256 in SEQ ID NO: 1; T, H, A, L, M at a residue corresponding to position 258 in SEQ ID NO: 1; A at a residue corresponding to position 259 in SEQ ID NO: 1; K, C, N at a residue corresponding to position 262 in SEQ ID NO: 1; F at a residue corresponding to position 268 in SEQ ID NO: 1; R, Q at a residue corresponding to position 271 in SEQ ID NO: 1; E at a residue corresponding to position 275 in SEQ ID NO: 1; L, M at a residue corresponding to position 277 in SEQ ID NO: 1; V at a residue corresponding to position 281 in SEQ ID NO: 1; V, I, F, T, H, Y at a residue corresponding to position 288 in SEQ ID NO: 1; V, A at a residue corresponding to position 290 in SEQ ID NO: 1; L, M at a residue corresponding to position 296 in SEQ ID NO: 1; I at a residue corresponding to position 297 in SEQ ID NO: 1; A at a residue corresponding to position 300 in SEQ ID NO: 1; E at a residue corresponding to position 304 in SEQ ID NO: 1; D at a residue corresponding to position 306 in SEQ ID NO: 1; L at a residue corresponding to position 316 in SEQ ID NO: 1; S, R, H, W, Y, M, T, C, Q, V, I, L at a residue corresponding to position 318 in SEQ ID NO: 1; M, F, T, Y, W at a residue corresponding to position 320 in SEQ ID NO: 1; C at a residue corresponding to position 321 in SEQ ID NO: 1; G at a residue corresponding to position 323 in SEQ ID NO: 1; V, H, R at a residue corresponding to position 328 in SEQ ID NO: 1; I, L at a residue corresponding to position 330 in SEQ ID NO: 1; I at a residue corresponding to position 331 in SEQ ID NO: 1; K at a residue corresponding to position 336 in SEQ ID NO: 1; K at a residue corresponding to position 352 in SEQ ID NO: 1; V, L at a residue corresponding to position 353 in SEQ ID NO: 1; G at a residue corresponding to position 363 in SEQ ID NO: 1; S at a residue corresponding to position 364 in SEQ ID NO: 1; C, L, I, M at a residue corresponding to position 365 in SEQ ID NO: 1; V at a residue corresponding to position 366 in SEQ ID NO: 1; H, S, T, M at a residue corresponding to position 369 in SEQ ID NO: 1; S, K, H, N at a residue corresponding to position 372 in SEQ ID NO: 1; Q, R, T, P, H, S, A, D, E at a residue corresponding to position 374 in SEQ ID NO: 1; T at a residue corresponding to position 376 in SEQ ID NO: 1; A, P, S, V at a residue corresponding to position 378 in SEQ ID NO: 1; T at a residue corresponding to position 388 in SEQ ID NO: 1; C, A at a residue corresponding to position 390 in SEQ ID NO: 1; D at a residue corresponding to position 394 in SEQ ID NO: 1; N at a residue corresponding to position 398 in SEQ ID NO: 1; L at a residue corresponding to position 406 in SEQ ID NO: 1; D at a residue corresponding to position 407 in SEQ ID NO: 1; N, E at a residue corresponding to position 416 in SEQ ID NO: 1; F at a residue corresponding to position 432 in SEQ ID NO: 1; R, K at a residue corresponding to position 443 in SEQ ID NO: 1; A at a residue corresponding to position 445 in SEQ ID NO: 1; S, G at a residue corresponding to position 455 in SEQ ID NO: 1; R at a residue corresponding to position 462 in SEQ ID NO: 1; M at a residue corresponding to position 469 in SEQ ID NO: 1; G at a residue corresponding to position 470 in SEQ ID NO: 1; R, M, W at a residue corresponding to position 471 in SEQ ID NO: 1; V at a residue corresponding to position 480 in SEQ ID NO: 1; I, C at a residue corresponding to position 481 in SEQ ID NO: 1; L at a residue corresponding to position 484 in SEQ ID NO: 1; D at a residue corresponding to position 486 in SEQ ID NO: 1; C at a residue corresponding to position 490 in SEQ ID NO: 1; R, G at a residue corresponding to position 491 in SEQ ID NO: 1; L, H, Y at a residue corresponding to position 493 in SEQ ID NO: 1; N at a residue corresponding to position 494 in SEQ ID NO: 1; Y at a residue corresponding to position 496 in SEQ ID NO: 1; P at a residue corresponding to position 508 in SEQ ID NO: 1; A, V, C at a residue corresponding to position 513 in SEQ ID NO: 1; V at a residue corresponding to position 518 in SEQ ID NO: 1; P at a residue corresponding to position 519 in SEQ ID NO: 1; E, P at a residue corresponding to position 521 in SEQ ID NO: 1; R, Q, A, Y, M, D at a residue corresponding to position 523 in SEQ ID NO: 1; D, S, T, G, E at a residue corresponding to position 525 in SEQ ID NO: 1; K at a residue corresponding to position 546 in SEQ ID NO: 1; H, S, P at a residue corresponding to position 554 in SEQ ID NO: 1; F at a residue corresponding to position 555 in SEQ ID NO: 1; G at a residue corresponding to position 560 in SEQ ID NO: 1; V at a residue corresponding to position 565 in SEQ ID NO: 1; Q, E at a residue corresponding to position 583 in SEQ ID NO: 1; or any combination thereof. It is understood that when alterations are listed herein for a certain position, the alteration can be in the alternative or in the conjunctive depending on the context. For example, the S, R, H, W, Y, M, T, C, Q, V, I, L at a residue corresponding to position 318 in SEQ ID NO: 1, means that alterations at a residue corresponding to position 318 in SEQ ID NO: 1 can comprise S or R or H or W or Y or M or T or C or Q or V or I or L.

In certain embodiments, the one or more amino acid alterations of the engineered ACL is an alteration described in TABLE 9, which results in the engineered ACL having greater or similar activity on HHA, as compared to the WT, when the engineered ACL is expressed in an organism having a pathway for production of HHA and assayed under conditions as described in Example 4. For example, in some embodiments, the one or more amino acid alterations result in an engineered ACL having: E, R at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; D, N, R, P, K at a residue corresponding to position 10 in SEQ ID NO: 2; A, V, M, I at a residue corresponding to position 12 in SEQ ID NO: 2; C at a residue corresponding to position 13 in SEQ ID NO: 2; Q, P, A at a residue corresponding to position 14 in SEQ ID NO: 2; S, E at a residue corresponding to position 15 in SEQ ID NO: 2; I, C at a residue corresponding to position 16 in SEQ ID NO: 2; D, R at a residue corresponding to position 19 in SEQ ID NO: 2; H, K, R, D at a residue corresponding to position 20 in SEQ ID NO: 2; E, D at a residue corresponding to position 22 in SEQ ID NO: 2; N at a residue corresponding to position 25 in SEQ ID NO: 2; M at a residue corresponding to position 28 in SEQ ID NO: 2; C at a residue corresponding to position 37 in SEQ ID NO: 2; R at a residue corresponding to position 39 in SEQ ID NO: 2; T at a residue corresponding to position 40 in SEQ ID NO: 2; V, I, M at a residue corresponding to position 41 in SEQ ID NO: 2; S at a residue corresponding to position 43 in SEQ ID NO: 2; I, V at a residue corresponding to position 45 in SEQ ID NO: 2; R, L, I, V, F at a residue corresponding to position 50 in SEQ ID NO: 2; R at a residue corresponding to position 51 in SEQ ID NO: 2; L at a residue corresponding to position 53 in SEQ ID NO: 2; F at a residue corresponding to position 57 in SEQ ID NO: 2; F, M, L, V at a residue corresponding to position 58 in SEQ ID NO: 2; V at a residue corresponding to position 59 in SEQ ID NO: 2; P at a residue corresponding to position 61 in SEQ ID NO: 2; D at a residue corresponding to position 62 in SEQ ID NO: 2; D at a residue corresponding to position 63 in SEQ ID NO: 2; K at a residue corresponding to position 67 in SEQ ID NO: 2; F, L, Y at a residue corresponding to position 69 in SEQ ID NO: 2; W, F at a residue corresponding to position 71 in SEQ ID NO: 2; A, R at a residue corresponding to position 72 in SEQ ID NO: 2; F at a residue corresponding to position 74 in SEQ ID NO: 2; D at a residue corresponding to position 75 in SEQ ID NO: 2; D, T, A, R at a residue corresponding to position 76 in SEQ ID NO: 2; R at a residue corresponding to position 77 in SEQ ID NO: 2; A at a residue corresponding to position 78 in SEQ ID NO: 2; K at a residue corresponding to position 80 in SEQ ID NO: 2; C at a residue corresponding to position 81 in SEQ ID NO: 2; S, N at a residue corresponding to position 83 in SEQ ID NO: 2; Y, H at a residue corresponding to position 84 in SEQ ID NO: 2; F at a residue corresponding to position 85 in SEQ ID NO: 2; Q, A at a residue corresponding to position 86 in SEQ ID NO: 2; R at a residue corresponding to position 87 in SEQ ID NO: 2; L at a residue corresponding to position 88 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; A at a residue corresponding to position 97 in SEQ ID NO: 2; M at a residue corresponding to position 98 in SEQ ID NO: 2; L at a residue corresponding to position 99 in SEQ ID NO: 2; C at a residue corresponding to position 100 in SEQ ID NO: 2; P at a residue corresponding to position 101 in SEQ ID NO: 2; R at a residue corresponding to position 103 in SEQ ID NO: 2; A, L, W at a residue corresponding to position 104 in SEQ ID NO: 2; F at a residue corresponding to position 106 in SEQ ID NO: 2; V at a residue corresponding to position 108 in SEQ ID NO: 2; V at a residue corresponding to position 109 in SEQ ID NO: 2; A, M at a residue corresponding to position 110 in SEQ ID NO: 2; C at a residue corresponding to position 113 in SEQ ID NO: 2; I at a residue corresponding to position 116 in SEQ ID NO: 2; T, V, I at a residue corresponding to position 119 in SEQ ID NO: 2; Y, V, F, L, M, A at a residue corresponding to position 120 in SEQ ID NO: 2; T at a residue corresponding to position 121 in SEQ ID NO: 2; I, M, V at a residue corresponding to position 123 in SEQ ID NO: 2; S, N, Y at a residue corresponding to position 124 in SEQ ID NO: 2; P, T, W at a residue corresponding to position 125 in SEQ ID NO: 2; R, A, N, M at a residue corresponding to position 126 in SEQ ID NO: 2; L, W, F at a residue corresponding to position 127 in SEQ ID NO: 2; R at a residue corresponding to position 128 in SEQ ID NO: 2; M at a residue corresponding to position 130 in SEQ ID NO: 2; E at a residue corresponding to position 131 in SEQ ID NO: 2; E, K, Q at a residue corresponding to position 136 in SEQ ID NO: 2; M at a residue corresponding to position 137 in SEQ ID NO: 2; M, I, C at a residue corresponding to position 140 in SEQ ID NO: 2; M at a residue corresponding to position 141 in SEQ ID NO: 2; Q, N at a residue corresponding to position 142 in SEQ ID NO: 2; R, D, G at a residue corresponding to position 145 in SEQ ID NO: 2; S at a residue corresponding to position 147 in SEQ ID NO: 2; V, L, A at a residue corresponding to position 148 in SEQ ID NO: 2; V at a residue corresponding to position 149 in SEQ ID NO: 2; V at a residue corresponding to position 150 in SEQ ID NO: 2; C at a residue corresponding to position 151 in SEQ ID NO: 2; E, R at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 165 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, L, V at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 189 in SEQ ID NO: 2; V at a residue corresponding to position 190 in SEQ ID NO: 2; E, A at a residue corresponding to position 191 in SEQ ID NO: 2; T at a residue corresponding to position 192 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; P at a residue corresponding to position 196 in SEQ ID NO: 2; L, A at a residue corresponding to position 201 in SEQ ID NO: 2; T at a residue corresponding to position 205 in SEQ ID NO: 2; D at a residue corresponding to position 208 in SEQ ID NO: 2; I, T, V at a residue corresponding to position 209 in SEQ ID NO: 2; L at a residue corresponding to position 211 in SEQ ID NO: 2; L, M at a residue corresponding to position 212 in SEQ ID NO: 2; I, M, V at a residue corresponding to position 213 in SEQ ID NO: 2; Y at a residue corresponding to position 214 in SEQ ID NO: 2; S at a residue corresponding to position 215 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; K, L, T, H, R, M at a residue corresponding to position 221 in SEQ ID NO: 2; S at a residue corresponding to position 222 in SEQ ID NO: 2; A at a residue corresponding to position 224 in SEQ ID NO: 2; M, V, I at a residue corresponding to position 226 in SEQ ID NO: 2; I at a residue corresponding to position 227 in SEQ ID NO: 2; S at a residue corresponding to position 228 in SEQ ID NO: 2; H, K at a residue corresponding to position 230 in SEQ ID NO: 2; L at a residue corresponding to position 232 in SEQ ID NO: 2; A at a residue corresponding to position 233 in SEQ ID NO: 2; S at a residue corresponding to position 234 in SEQ ID NO: 2; Q at a residue corresponding to position 235 in SEQ ID NO: 2; M at a residue corresponding to position 236 in SEQ ID NO: 2; A, M, Q at a residue corresponding to position 237 in SEQ ID NO: 2; M, H at a residue corresponding to position 238 in SEQ ID NO: 2; V, L, M at a residue corresponding to position 239 in SEQ ID NO: 2; A at a residue corresponding to position 240 in SEQ ID NO: 2; Q, E, H, A at a residue corresponding to position 241 in SEQ ID NO: 2; C, V, T at a residue corresponding to position 242 in SEQ ID NO: 2; W, F, M, I, V at a residue corresponding to position 243 in SEQ ID NO: 2; P, Q, G, H, E at a residue corresponding to position 244 in SEQ ID NO: 2; L, M, W at a residue corresponding to position 245 in SEQ ID NO: 2; I at a residue corresponding to position 246 in SEQ ID NO: 2; R, P at a residue corresponding to position 247 in SEQ ID NO: 2; V, H at a residue corresponding to position 252 in SEQ ID NO: 2; L, V, M at a residue corresponding to position 253 in SEQ ID NO: 2; M, I, V at a residue corresponding to position 254 in SEQ ID NO: 2; C, V, T, N at a residue corresponding to position 255 in SEQ ID NO: 2; Y, V, F at a residue corresponding to position 256 in SEQ ID NO: 2; S, H, F, Y at a residue corresponding to position 260 in SEQ ID NO: 2; T, C, A at a residue corresponding to position 262 in SEQ ID NO: 2; Y at a residue corresponding to position 263 in SEQ ID NO: 2; A at a residue corresponding to position 264 in SEQ ID NO: 2; M at a residue corresponding to position 265 in SEQ ID NO: 2; N, M at a residue corresponding to position 266 in SEQ ID NO: 2; M, F, L, W, G at a residue corresponding to position 267 in SEQ ID NO: 2; M, A at a residue corresponding to position 268 in SEQ ID NO: 2; M at a residue corresponding to position 270 in SEQ ID NO: 2; L, C, A at a residue corresponding to position 271 in SEQ ID NO: 2; A, V, T, P, C at a residue corresponding to position 272 in SEQ ID NO: 2; I, V, M, F at a residue corresponding to position 273 in SEQ ID NO: 2; C, M, H, R, Y at a residue corresponding to position 274 in SEQ ID NO: 2; H, C at a residue corresponding to position 275 in SEQ ID NO: 2; A at a residue corresponding to position 277 in SEQ ID NO: 2; T, A, C at a residue corresponding to position 278 in SEQ ID NO: 2; M, I, V at a residue corresponding to position 279 in SEQ ID NO: 2; V, L at a residue corresponding to position 281 in SEQ ID NO: 2; E, D, P, R at a residue corresponding to position 283 in SEQ ID NO: 2; R, F, D at a residue corresponding to position 285 in SEQ ID NO: 2; D, F at a residue corresponding to position 286 in SEQ ID NO: 2; A, P, V at a residue corresponding to position 287 in SEQ ID NO: 2; A at a residue corresponding to position 290 in SEQ ID NO: 2; M, I, L, V at a residue corresponding to position 291 in SEQ ID NO: 2; H at a residue corresponding to position 292 in SEQ ID NO: 2; A at a residue corresponding to position 294 in SEQ ID NO: 2; V, I, M at a residue corresponding to position 295 in SEQ ID NO: 2; Q, D at a residue corresponding to position 296 in SEQ ID NO: 2; M, V, L at a residue corresponding to position 298 in SEQ ID NO: 2; A at a residue corresponding to position 299 in SEQ ID NO: 2; Q at a residue corresponding to position 300 in SEQ ID NO: 2; Q at a residue corresponding to position 302 in SEQ ID NO: 2; A, P, C, V at a residue corresponding to position 303 in SEQ ID NO: 2; S at a residue corresponding to position 304 in SEQ ID NO: 2; V, M, I at a residue corresponding to position 305 in SEQ ID NO: 2; I, V, Y at a residue corresponding to position 306 in SEQ ID NO: 2; M, W, P at a residue corresponding to position 307 in SEQ ID NO: 2; G at a residue corresponding to position 308 in SEQ ID NO: 2; T, A, R at a residue corresponding to position 311 in SEQ ID NO: 2; F, V, M at a residue corresponding to position 312 in SEQ ID NO: 2; F, W at a residue corresponding to position 313 in SEQ ID NO: 2; Q, H, E at a residue corresponding to position 314 in SEQ ID NO: 2; A, K, L, R at a residue corresponding to position 315 in SEQ ID NO: 2; I, M at a residue corresponding to position 316 in SEQ ID NO: 2; D, H at a residue corresponding to position 318 in SEQ ID NO: 2; R, Q at a residue corresponding to position 321 in SEQ ID NO: 2; A, K, H, Q at a residue corresponding to position 322 in SEQ ID NO: 2; F at a residue corresponding to position 330 in SEQ ID NO: 2; G at a residue corresponding to position 331 in SEQ ID NO: 2; S at a residue corresponding to position 333 in SEQ ID NO: 2; V, I, M at a residue corresponding to position 334 in SEQ ID NO: 2; K at a residue corresponding to position 335 in SEQ ID NO: 2; V, T, M, Y, W, I, F at a residue corresponding to position 336 in SEQ ID NO: 2; L, V, M at a residue corresponding to position 337 in SEQ ID NO: 2; L, M, V, W at a residue corresponding to position 338 in SEQ ID NO: 2; T, S, F at a residue corresponding to position 339 in SEQ ID NO: 2; G at a residue corresponding to position 340 in SEQ ID NO: 2; S at a residue corresponding to position 342 in SEQ ID NO: 2; P, A at a residue corresponding to position 343 in SEQ ID NO: 2; I, C, M, V at a residue corresponding to position 344 in SEQ ID NO: 2; S at a residue corresponding to position 345 in SEQ ID NO: 2; E, V, A, P at a residue corresponding to position 346 in SEQ ID NO: 2; E at a residue corresponding to position 347 in SEQ ID NO: 2; I, L, T at a residue corresponding to position 348 in SEQ ID NO: 2; Y, F, L at a residue corresponding to position 349 in SEQ ID NO: 2; R, Q, W at a residue corresponding to position 351 in SEQ ID NO: 2; I, M at a residue corresponding to position 352 in SEQ ID NO: 2; R at a residue corresponding to position 353 in SEQ ID NO: 2; K at a residue corresponding to position 354 in SEQ ID NO: 2; A at a residue corresponding to position 355 in SEQ ID NO: 2; F, I, V, A, G, L, W at a residue corresponding to position 357 in SEQ ID NO: 2; Q at a residue corresponding to position 358 in SEQ ID NO: 2; Q at a residue corresponding to position 359 in SEQ ID NO: 2; C, H, L at a residue corresponding to position 362 in SEQ ID NO: 2; V, I, G at a residue corresponding to position 363 in SEQ ID NO: 2; M, I, V at a residue corresponding to position 365 in SEQ ID NO: 2; I, L, F, V at a residue corresponding to position 366 in SEQ ID NO: 2; V, I, L, M, T, C at a residue corresponding to position 367 in SEQ ID NO: 2; N at a residue corresponding to position 368 in SEQ ID NO: 2; A, G at a residue corresponding to position 369 in SEQ ID NO: 2; Y at a residue corresponding to position 370 in SEQ ID NO: 2; C, A at a residue corresponding to position 375 in SEQ ID NO: 2; G, S at a residue corresponding to position 376 in SEQ ID NO: 2; G at a residue corresponding to position 377 in SEQ ID NO: 2; T at a residue corresponding to position 378 in SEQ ID NO: 2; V, C, L, A, I at a residue corresponding to position 379 in SEQ ID NO: 2; S, V, H, T at a residue corresponding to position 380 in SEQ ID NO: 2; L, C at a residue corresponding to position 381 in SEQ ID NO: 2; T, C, N, L, P at a residue corresponding to position 382 in SEQ ID NO: 2; Y, R at a residue corresponding to position 383 in SEQ ID NO: 2; N, W, D at a residue corresponding to position 384 in SEQ ID NO: 2; G at a residue corresponding to position 385 in SEQ ID NO: 2; V, T, L at a residue corresponding to position 386 in SEQ ID NO: 2; R, H, N, D, E at a residue corresponding to position 387 in SEQ ID NO: 2; K, H at a residue corresponding to position 388 in SEQ ID NO: 2; A, P, L at a residue corresponding to position 389 in SEQ ID NO: 2; S, T at a residue corresponding to position 391 in SEQ ID NO: 2; V at a residue corresponding to position 392 in SEQ ID NO: 2; I, K at a residue corresponding to position 394 in SEQ ID NO: 2; V at a residue corresponding to position 396 in SEQ ID NO: 2; H at a residue corresponding to position 398 in SEQ ID NO: 2; C, I, T, V at a residue corresponding to position 399 in SEQ ID NO: 2; Q, D at a residue corresponding to position 400 in SEQ ID NO: 2; L, I, C, A, M at a residue corresponding to position 401 in SEQ ID NO: 2; V at a residue corresponding to position 404 in SEQ ID NO: 2; E at a residue corresponding to position 406 in SEQ ID NO: 2; P at a residue corresponding to position 407 in SEQ ID NO: 2; G, Q, E at a residue corresponding to position 409 in SEQ ID NO: 2; S at a residue corresponding to position 411 in SEQ ID NO: 2; L, C at a residue corresponding to position 414 in SEQ ID NO: 2; R at a residue corresponding to position 416 in SEQ ID NO: 2; Q, H, M, S at a residue corresponding to position 419 in SEQ ID NO: 2; V at a residue corresponding to position 420 in SEQ ID NO: 2; F, T at a residue corresponding to position 421 in SEQ ID NO: 2; L, H at a residue corresponding to position 425 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; V, T at a residue corresponding to position 434 in SEQ ID NO: 2; M at a residue corresponding to position 435 in SEQ ID NO: 2; F, M at a residue corresponding to position 441 in SEQ ID NO: 2; Y, R, K, C, H at a residue corresponding to position 442 in SEQ ID NO: 2; S at a residue corresponding to position 443 in SEQ ID NO: 2; L, V, M, I at a residue corresponding to position 446 in SEQ ID NO: 2; A at a residue corresponding to position 447 in SEQ ID NO: 2; M, D at a residue corresponding to position 450 in SEQ ID NO: 2; E at a residue corresponding to position 451 in SEQ ID NO: 2; P at a residue corresponding to position 452 in SEQ ID NO: 2; G at a residue corresponding to position 453 in SEQ ID NO: 2; N, D at a residue corresponding to position 456 in SEQ ID NO: 2; F, I, R, W, Y at a residue corresponding to position 460 in SEQ ID NO: 2; Y, L at a residue corresponding to position 461 in SEQ ID NO: 2; V, I at a residue corresponding to position 462 in SEQ ID NO: 2; D at a residue corresponding to position 463 in SEQ ID NO: 2; L at a residue corresponding to position 465 in SEQ ID NO: 2; A, D, K at a residue corresponding to position 466 in SEQ ID NO: 2; K at a residue corresponding to position 467 in SEQ ID NO: 2; M at a residue corresponding to position 468 in SEQ ID NO: 2; L at a residue corresponding to position 469 in SEQ ID NO: 2; V at a residue corresponding to position 470 in SEQ ID NO: 2; T, V at a residue corresponding to position 471 in SEQ ID NO: 2; N, S at a residue corresponding to position 473 in SEQ ID NO: 2; Y, F, V, L, I, N at a residue corresponding to position 476 in SEQ ID NO: 2; T at a residue corresponding to position 478 in SEQ ID NO: 2; V, P at a residue corresponding to position 479 in SEQ ID NO: 2; S, A, P, L at a residue corresponding to position 480 in SEQ ID NO: 2; P, E at a residue corresponding to position 481 in SEQ ID NO: 2; L, E, R, A at a residue corresponding to position 485 in SEQ ID NO: 2; C at a residue corresponding to position 490 in SEQ ID NO: 2; P at a residue corresponding to position 492 in SEQ ID NO: 2; C at a residue corresponding to position 498 in SEQ ID NO: 2; V at a residue corresponding to position 507 in SEQ ID NO: 2; V at a residue corresponding to position 508 in SEQ ID NO: 2; A at a residue corresponding to position 510 in SEQ ID NO: 2; V, L at a residue corresponding to position 511 in SEQ ID NO: 2; I at a residue corresponding to position 512 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; M, L at a residue corresponding to position 526 in SEQ ID NO: 2; L at a residue corresponding to position 527 in SEQ ID NO: 2; R, H at a residue corresponding to position 532 in SEQ ID NO: 2; L at a residue corresponding to position 535 in SEQ ID NO: 2; Q at a residue corresponding to position 540 in SEQ ID NO: 2; Y, W at a residue corresponding to position 542 in SEQ ID NO: 2; N at a residue corresponding to position 546 in SEQ ID NO: 2; Q at a residue corresponding to position 552 in SEQ ID NO: 2; E at a residue corresponding to position 554 in SEQ ID NO: 2; D at a residue corresponding to position 560 in SEQ ID NO: 2; D at a residue corresponding to position 561 in SEQ ID NO: 2; L, F at a residue corresponding to position 563 in SEQ ID NO: 2; S, T, L, D, R, K at a residue corresponding to position 564 in SEQ ID NO: 2; T, M, P, V, I at a residue corresponding to position 565 in SEQ ID NO: 2; D, A, E, P at a residue corresponding to position 566 in SEQ ID NO: 2; A, S, Q, N, T, K at a residue corresponding to position 567 in SEQ ID NO: 2; G, D, N at a residue corresponding to position 568 in SEQ ID NO: 2; M, K at a residue corresponding to position 569 in SEQ ID NO: 2; V, L at a residue corresponding to position 570 in SEQ ID NO: 2; P at a residue corresponding to position 572 in SEQ ID NO: 2; S, T at a residue corresponding to position 573 in SEQ ID NO: 2; M, L at a residue corresponding to position 574 in SEQ ID NO: 2; T, Y at a residue corresponding to position 575 in SEQ ID NO: 2; A, L at a residue corresponding to position 577 in SEQ ID NO: 2; A, Q at a residue corresponding to position 578 in SEQ ID NO: 2; D at a residue corresponding to position 583 in SEQ ID NO: 2; N at a residue corresponding to position 584 in SEQ ID NO: 2; H at a residue corresponding to position 585 in SEQ ID NO: 2; A at a residue corresponding to position 587 in SEQ ID NO: 2; Q at a residue corresponding to position 588 in SEQ ID NO: 2; Q, E at a residue corresponding to position 591 in SEQ ID NO: 2; A at a residue corresponding to position 593 in SEQ ID NO: 2; E at a residue corresponding to position 595 in SEQ ID NO: 2; K at a residue corresponding to position 596 in SEQ ID NO: 2; D at a residue corresponding to position 598 in SEQ ID NO: 2; S at a residue corresponding to position 599 in SEQ ID NO: 2; L at a residue corresponding to position 601 in SEQ ID NO: 2; or any combination thereof. It is understood that when alterations are listed herein for a certain position, the alteration can be in the alternative or in the conjunctive depending on the context. For example, the F, L, Y at a residue corresponding to position 69 in SEQ ID NO: 2, means that alterations at a residue corresponding to position 69 in SEQ ID NO: 2 can comprise F or L or Y.

In certain embodiments, the one or more amino acid alterations of the engineered ACL is an alteration described in TABLE 8, which results in the engineered ACL having about or greater than 2× the amount of activity on HHA, as compared to the WT, when the engineered ACL is expressed in an organism having a pathway for production of HHA and assayed under conditions as described in Example 4. For example, in some embodiments, the one or more amino acid alterations result in an engineered ACL having: F at a residue corresponding to position 10 in SEQ ID NO: 1; A at a residue corresponding to position 199 in SEQ ID NO: 1; L, H, A at a residue corresponding to position 223 in SEQ ID NO: 1; M at a residue corresponding to position 229 in SEQ ID NO: 1; Q, A at a residue corresponding to position 230 in SEQ ID NO: 1; P at a residue corresponding to position 231 in SEQ ID NO: 1; N at a residue corresponding to position 232 in SEQ ID NO: 1; W, Y at a residue corresponding to position 233 in SEQ ID NO: 1; H at a residue corresponding to position 234 in SEQ ID NO: 1; S at a residue corresponding to position 235 in SEQ ID NO: 1; T at a residue corresponding to position 238 in SEQ ID NO: 1; C, M at a residue corresponding to position 243 in SEQ ID NO: 1; I, V at a residue corresponding to position 256 in SEQ ID NO: 1; L, M at a residue corresponding to position 258 in SEQ ID NO: 1; M at a residue corresponding to position 277 in SEQ ID NO: 1; Y, H at a residue corresponding to position 288 in SEQ ID NO: 1; A at a residue corresponding to position 300 in SEQ ID NO: 1; V, T, C, I, L, Q, M, Y at a residue corresponding to position 318 in SEQ ID NO: 1; W, Y at a residue corresponding to position 320 in SEQ ID NO: 1; C at a residue corresponding to position 321 in SEQ ID NO: 1; G at a residue corresponding to position 323 in SEQ ID NO: 1; R at a residue corresponding to position 328 in SEQ ID NO: 1; L at a residue corresponding to position 330 in SEQ ID NO: 1; S at a residue corresponding to position 364 in SEQ ID NO: 1; I, M at a residue corresponding to position 365 in SEQ ID NO: 1; M at a residue corresponding to position 369 in SEQ ID NO: 1; N at a residue corresponding to position 372 in SEQ ID NO: 1; E at a residue corresponding to position 374 in SEQ ID NO: 1; V at a residue corresponding to position 378 in SEQ ID NO: 1; R at a residue corresponding to position 462 in SEQ ID NO: 1; M at a residue corresponding to position 469 in SEQ ID NO: 1; V at a residue corresponding to position 480 in SEQ ID NO: 1; C at a residue corresponding to position 481 in SEQ ID NO: 1; L at a residue corresponding to position 484 in SEQ ID NO: 1; C, A, V at a residue corresponding to position 513 in SEQ ID NO: 1; D at a residue corresponding to position 523 in SEQ ID NO: 1; G, E, T at a residue corresponding to position 525 in SEQ ID NO: 1; S, P at a residue corresponding to position 554 in SEQ ID NO: 1; G at a residue corresponding to position 560 in SEQ ID NO: 1; E at a residue corresponding to position 583 in SEQ ID NO: 1; or any combination thereof. It is understood that when alterations are listed herein for a certain position, the alteration can be in the alternative or in the conjunctive depending on the context. For example, the V, T, C, I, L, Q, M, Y at a residue corresponding to position 318 in SEQ ID NO: 1, means that alterations at a residue corresponding to position 318 in SEQ ID NO: 1 can comprise V or T or C or I or L or Q or M or Y.

In certain embodiments, the one or more amino acid alterations of the engineered ACL is an alteration described in TABLE 9, which results in the engineered ACL having about or greater than 2× the amount of activity on HHA, as compared to the WT, when the engineered ACL is expressed in an organism having a pathway for production of HHA and assayed under conditions as described in Example 4. For example, in some embodiments, the one or more amino acid alterations result in an engineered ACL having: E, R at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; D, N, R, P at a residue corresponding to position 10 in SEQ ID NO: 2; A, V, M at a residue corresponding to position 12 in SEQ ID NO: 2; Q, P at a residue corresponding to position 14 in SEQ ID NO: 2; S, E at a residue corresponding to position 15 in SEQ ID NO: 2; I at a residue corresponding to position 16 in SEQ ID NO: 2; D, R at a residue corresponding to position 19 in SEQ ID NO: 2; H, K, R, D at a residue corresponding to position 20 in SEQ ID NO: 2; E, D at a residue corresponding to position 22 in SEQ ID NO: 2; M at a residue corresponding to position 28 in SEQ ID NO: 2; R at a residue corresponding to position 39 in SEQ ID NO: 2; V at a residue corresponding to position 41 in SEQ ID NO: 2; S at a residue corresponding to position 43 in SEQ ID NO: 2; I at a residue corresponding to position 45 in SEQ ID NO: 2; R, L, I, V, F at a residue corresponding to position 50 in SEQ ID NO: 2; F at a residue corresponding to position 57 in SEQ ID NO: 2; F, M, L, V at a residue corresponding to position 58 in SEQ ID NO: 2; P at a residue corresponding to position 61 in SEQ ID NO: 2; D at a residue corresponding to position 62 in SEQ ID NO: 2; D at a residue corresponding to position 63 in SEQ ID NO: 2; K at a residue corresponding to position 67 in SEQ ID NO: 2; F, L, Y at a residue corresponding to position 69 in SEQ ID NO: 2; W, F at a residue corresponding to position 71 in SEQ ID NO: 2; A, R at a residue corresponding to position 72 in SEQ ID NO: 2; D, T, A, R at a residue corresponding to position 76 in SEQ ID NO: 2; R at a residue corresponding to position 77 in SEQ ID NO: 2; A at a residue corresponding to position 78 in SEQ ID NO: 2; C at a residue corresponding to position 81 in SEQ ID NO: 2; Y at a residue corresponding to position 84 in SEQ ID NO: 2; F at a residue corresponding to position 85 in SEQ ID NO: 2; Q at a residue corresponding to position 86 in SEQ ID NO: 2; R at a residue corresponding to position 87 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; M at a residue corresponding to position 98 in SEQ ID NO: 2; A at a residue corresponding to position 104 in SEQ ID NO: 2; V at a residue corresponding to position 108 in SEQ ID NO: 2; V at a residue corresponding to position 109 in SEQ ID NO: 2; C at a residue corresponding to position 113 in SEQ ID NO: 2; I at a residue corresponding to position 116 in SEQ ID NO: 2; T at a residue corresponding to position 119 in SEQ ID NO: 2; Y, V, F, L, M, A at a residue corresponding to position 120 in SEQ ID NO: 2; I at a residue corresponding to position 123 in SEQ ID NO: 2; S, N at a residue corresponding to position 124 in SEQ ID NO: 2; P, T, W at a residue corresponding to position 125 in SEQ ID NO: 2; R, A, N at a residue corresponding to position 126 in SEQ ID NO: 2; L, W at a residue corresponding to position 127 in SEQ ID NO: 2; M at a residue corresponding to position 130 in SEQ ID NO: 2; E at a residue corresponding to position 131 in SEQ ID NO: 2; E, K at a residue corresponding to position 136 in SEQ ID NO: 2; M, I, C at a residue corresponding to position 140 in SEQ ID NO: 2; M at a residue corresponding to position 141 in SEQ ID NO: 2; Q, N at a residue corresponding to position 142 in SEQ ID NO: 2; R at a residue corresponding to position 145 in SEQ ID NO: 2; S at a residue corresponding to position 147 in SEQ ID NO: 2; V at a residue corresponding to position 148 in SEQ ID NO: 2; V at a residue corresponding to position 149 in SEQ ID NO: 2; C at a residue corresponding to position 151 in SEQ ID NO: 2; E, R at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, L, V at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 190 in SEQ ID NO: 2; E, A at a residue corresponding to position 191 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; L, A at a residue corresponding to position 201 in SEQ ID NO: 2; T at a residue corresponding to position 205 in SEQ ID NO: 2; I at a residue corresponding to position 209 in SEQ ID NO: 2; Y at a residue corresponding to position 214 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; K, L, T, H at a residue corresponding to position 221 in SEQ ID NO: 2; M, V at a residue corresponding to position 226 in SEQ ID NO: 2; S at a residue corresponding to position 228 in SEQ ID NO: 2; H, K at a residue corresponding to position 230 in SEQ ID NO: 2; L at a residue corresponding to position 232 in SEQ ID NO: 2; S at a residue corresponding to position 234 in SEQ ID NO: 2; A at a residue corresponding to position 237 in SEQ ID NO: 2; M at a residue corresponding to position 238 in SEQ ID NO: 2; V at a residue corresponding to position 239 in SEQ ID NO: 2; Q, E at a residue corresponding to position 241 in SEQ ID NO: 2; C, V, T at a residue corresponding to position 242 in SEQ ID NO: 2; W, F, M at a residue corresponding to position 243 in SEQ ID NO: 2; P, Q at a residue corresponding to position 244 in SEQ ID NO: 2; I at a residue corresponding to position 246 in SEQ ID NO: 2; R at a residue corresponding to position 247 in SEQ ID NO: 2; L, V at a residue corresponding to position 253 in SEQ ID NO: 2; M, I, V at a residue corresponding to position 254 in SEQ ID NO: 2; S, H at a residue corresponding to position 260 in SEQ ID NO: 2; T at a residue corresponding to position 262 in SEQ ID NO: 2; Y at a residue corresponding to position 263 in SEQ ID NO: 2; N at a residue corresponding to position 266 in SEQ ID NO: 2; M, F at a residue corresponding to position 267 in SEQ ID NO: 2; A at a residue corresponding to position 272 in SEQ ID NO: 2; I, V, M, F at a residue corresponding to position 273 in SEQ ID NO: 2; C at a residue corresponding to position 274 in SEQ ID NO: 2; T, A at a residue corresponding to position 278 in SEQ ID NO: 2; M at a residue corresponding to position 279 in SEQ ID NO: 2; V at a residue corresponding to position 281 in SEQ ID NO: 2; E at a residue corresponding to position 283 in SEQ ID NO: 2; R at a residue corresponding to position 285 in SEQ ID NO: 2; A at a residue corresponding to position 290 in SEQ ID NO: 2; M, I, L, V at a residue corresponding to position 291 in SEQ ID NO: 2; H at a residue corresponding to position 292 in SEQ ID NO: 2; V, I, M at a residue corresponding to position 295 in SEQ ID NO: 2; Q at a residue corresponding to position 296 in SEQ ID NO: 2; M, V at a residue corresponding to position 298 in SEQ ID NO: 2; Q at a residue corresponding to position 300 in SEQ ID NO: 2; Q at a residue corresponding to position 302 in SEQ ID NO: 2; A, P at a residue corresponding to position 303 in SEQ ID NO: 2; S at a residue corresponding to position 304 in SEQ ID NO: 2; V, M, I at a residue corresponding to position 305 in SEQ ID NO: 2; M at a residue corresponding to position 307 in SEQ ID NO: 2; T, A at a residue corresponding to position 311 in SEQ ID NO: 2; F at a residue corresponding to position 313 in SEQ ID NO: 2; Q, H at a residue corresponding to position 314 in SEQ ID NO: 2; A, K, L, R at a residue corresponding to position 315 in SEQ ID NO: 2; I at a residue corresponding to position 316 in SEQ ID NO: 2; D, H at a residue corresponding to position 318 in SEQ ID NO: 2; R, Q at a residue corresponding to position 321 in SEQ ID NO: 2; A, K, H, Q at a residue corresponding to position 322 in SEQ ID NO: 2; F at a residue corresponding to position 330 in SEQ ID NO: 2; S at a residue corresponding to position 333 in SEQ ID NO: 2; V, I at a residue corresponding to position 334 in SEQ ID NO: 2; V, T, M, Y at a residue corresponding to position 336 in SEQ ID NO: 2; L, V at a residue corresponding to position 337 in SEQ ID NO: 2; L, M, V at a residue corresponding to position 338 in SEQ ID NO: 2; T at a residue corresponding to position 339 in SEQ ID NO: 2; G at a residue corresponding to position 340 in SEQ ID NO: 2; I, C, M at a residue corresponding to position 344 in SEQ ID NO: 2; S at a residue corresponding to position 345 in SEQ ID NO: 2; E at a residue corresponding to position 346 in SEQ ID NO: 2; E at a residue corresponding to position 347 in SEQ ID NO: 2; I, L, T at a residue corresponding to position 348 in SEQ ID NO: 2; Y, F at a residue corresponding to position 349 in SEQ ID NO: 2; R, Q at a residue corresponding to position 351 in SEQ ID NO: 2; I at a residue corresponding to position 352 in SEQ ID NO: 2; R at a residue corresponding to position 353 in SEQ ID NO: 2; K at a residue corresponding to position 354 in SEQ ID NO: 2; A at a residue corresponding to position 355 in SEQ ID NO: 2; F, I, V, A, G, L, W at a residue corresponding to position 357 in SEQ ID NO: 2; Q at a residue corresponding to position 358 in SEQ ID NO: 2; Q at a residue corresponding to position 359 in SEQ ID NO: 2; C, H at a residue corresponding to position 362 in SEQ ID NO: 2; V, I at a residue corresponding to position 363 in SEQ ID NO: 2; M at a residue corresponding to position 365 in SEQ ID NO: 2; V, I, L, M at a residue corresponding to position 367 in SEQ ID NO: 2; A at a residue corresponding to position 369 in SEQ ID NO: 2; C at a residue corresponding to position 375 in SEQ ID NO: 2; G, S at a residue corresponding to position 376 in SEQ ID NO: 2; V, C, L at a residue corresponding to position 379 in SEQ ID NO: 2; T, C at a residue corresponding to position 382 in SEQ ID NO: 2; Y at a residue corresponding to position 383 in SEQ ID NO: 2; N at a residue corresponding to position 384 in SEQ ID NO: 2; V, T at a residue corresponding to position 386 in SEQ ID NO: 2; R, H, N, D at a residue corresponding to position 387 in SEQ ID NO: 2; K, H at a residue corresponding to position 388 in SEQ ID NO: 2; A, P, L at a residue corresponding to position 389 in SEQ ID NO: 2; C, I, T, V at a residue corresponding to position 399 in SEQ ID NO: 2; Q at a residue corresponding to position 400 in SEQ ID NO: 2; L, I, C at a residue corresponding to position 401 in SEQ ID NO: 2; V at a residue corresponding to position 404 in SEQ ID NO: 2; P at a residue corresponding to position 407 in SEQ ID NO: 2; G at a residue corresponding to position 409 in SEQ ID NO: 2; R at a residue corresponding to position 416 in SEQ ID NO: 2; Q at a residue corresponding to position 419 in SEQ ID NO: 2; V at a residue corresponding to position 420 in SEQ ID NO: 2; F at a residue corresponding to position 421 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; V at a residue corresponding to position 434 in SEQ ID NO: 2; F at a residue corresponding to position 441 in SEQ ID NO: 2; Y, R, K, C, H at a residue corresponding to position 442 in SEQ ID NO: 2; L, V, M, I at a residue corresponding to position 446 in SEQ ID NO: 2; M at a residue corresponding to position 450 in SEQ ID NO: 2; P at a residue corresponding to position 452 in SEQ ID NO: 2; G at a residue corresponding to position 453 in SEQ ID NO: 2; N, D at a residue corresponding to position 456 in SEQ ID NO: 2; F, I, R at a residue corresponding to position 460 in SEQ ID NO: 2; Y at a residue corresponding to position 461 in SEQ ID NO: 2; L at a residue corresponding to position 465 in SEQ ID NO: 2; A, D at a residue corresponding to position 466 in SEQ ID NO: 2; T at a residue corresponding to position 471 in SEQ ID NO: 2; N, S at a residue corresponding to position 473 in SEQ ID NO: 2; Y, F, V, L, I at a residue corresponding to position 476 in SEQ ID NO: 2; V, P at a residue corresponding to position 479 in SEQ ID NO: 2; S, A, P at a residue corresponding to position 480 in SEQ ID NO: 2; P, E at a residue corresponding to position 481 in SEQ ID NO: 2; L, E, R, A at a residue corresponding to position 485 in SEQ ID NO: 2; C at a residue corresponding to position 490 in SEQ ID NO: 2; P at a residue corresponding to position 492 in SEQ ID NO: 2; V at a residue corresponding to position 507 in SEQ ID NO: 2; V at a residue corresponding to position 511 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; M, L at a residue corresponding to position 526 in SEQ ID NO: 2; L at a residue corresponding to position 527 in SEQ ID NO: 2; R, H at a residue corresponding to position 532 in SEQ ID NO: 2; Q at a residue corresponding to position 540 in SEQ ID NO: 2; Y at a residue corresponding to position 542 in SEQ ID NO: 2; N at a residue corresponding to position 546 in SEQ ID NO: 2; E at a residue corresponding to position 554 in SEQ ID NO: 2; D at a residue corresponding to position 561 in SEQ ID NO: 2; S, T at a residue corresponding to position 564 in SEQ ID NO: 2; T, M, P, V, I at a residue corresponding to position 565 in SEQ ID NO: 2; D at a residue corresponding to position 566 in SEQ ID NO: 2; A, S, Q, N at a residue corresponding to position 567 in SEQ ID NO: 2; G, D, N at a residue corresponding to position 568 in SEQ ID NO: 2; V at a residue corresponding to position 570 in SEQ ID NO: 2; P at a residue corresponding to position 572 in SEQ ID NO: 2; M at a residue corresponding to position 574 in SEQ ID NO: 2; T at a residue corresponding to position 575 in SEQ ID NO: 2; A at a residue corresponding to position 577 in SEQ ID NO: 2; N at a residue corresponding to position 584 in SEQ ID NO: 2; A at a residue corresponding to position 587 in SEQ ID NO: 2; E at a residue corresponding to position 595 in SEQ ID NO: 2; K at a residue corresponding to position 596 in SEQ ID NO: 2; S at a residue corresponding to position 599 in SEQ ID NO: 2; or any combination thereof. It is understood that when alterations are listed herein for a certain position, the alteration can be in the alternative or in the conjunctive depending on the context. For example, the F, L, Y at a residue corresponding to position 69 in SEQ ID NO: 2, means that alterations at a residue corresponding to position 69 in SEQ ID NO: 2 can comprise F or L or Y.

In certain embodiments, the one or more amino acid alterations of the engineered ACL is an alteration described in TABLE 8, which results in the engineered ACL having about or greater than 3× the amount of activity on HHA, as compared to the WT, when the engineered ACL is expressed in an organism having a pathway for production of HHA and assayed under conditions as described in Example 4. For example, in some embodiments, the one or more amino acid alterations result in an engineered ACL having: L, H at a residue corresponding to position 223 in SEQ ID NO: 1; N at a residue corresponding to position 232 in SEQ ID NO: 1; W, Y at a residue corresponding to position 233 in SEQ ID NO: 1; H at a residue corresponding to position 234 in SEQ ID NO: 1; T at a residue corresponding to position 238 in SEQ ID NO: 1; L at a residue corresponding to position 258 in SEQ ID NO: 1; Y, H at a residue corresponding to position 288 in SEQ ID NO: 1; V, T, C, I, L at a residue corresponding to position 318 in SEQ ID NO: 1; W, Y at a residue corresponding to position 320 in SEQ ID NO: 1; C at a residue corresponding to position 321 in SEQ ID NO: 1; G at a residue corresponding to position 323 in SEQ ID NO: 1; S at a residue corresponding to position 364 in SEQ ID NO: 1; C, A at a residue corresponding to position 513 in SEQ ID NO: 1; D at a residue corresponding to position 523 in SEQ ID NO: 1; or any combination thereof. It is understood that when alterations are listed herein for a certain position, the alteration can be in the alternative or in the conjunctive depending on the context. For example, the V, T, C, I, L at a residue corresponding to position 318 in SEQ ID NO: 1, means that alterations at a residue corresponding to position 318 in SEQ ID NO: 1 can comprise V or T or C or I or L.

In certain embodiments, the one or more amino acid alterations of the engineered ACL is an alteration described in TABLE 9, which results in the engineered ACL having about or greater than 3× the amount of activity on HHA, as compared to the WT, when the engineered ACL is expressed in an organism having a pathway for production of HHA and assayed under conditions as described in Example 4. For example, in some embodiments, the one or more amino acid alterations result in an engineered ACL having: E, R at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; D, N at a residue corresponding to position 10 in SEQ ID NO: 2; A at a residue corresponding to position 12 in SEQ ID NO: 2; Q, P at a residue corresponding to position 14 in SEQ ID NO: 2; S, E at a residue corresponding to position 15 in SEQ ID NO: 2; D at a residue corresponding to position 19 in SEQ ID NO: 2; H, K, R at a residue corresponding to position 20 in SEQ ID NO: 2; E at a residue corresponding to position 22 in SEQ ID NO: 2; V at a residue corresponding to position 41 in SEQ ID NO: 2; I at a residue corresponding to position 45 in SEQ ID NO: 2; R, L, I, V at a residue corresponding to position 50 in SEQ ID NO: 2; F, M, L at a residue corresponding to position 58 in SEQ ID NO: 2; D at a residue corresponding to position 62 in SEQ ID NO: 2; D at a residue corresponding to position 63 in SEQ ID NO: 2; K at a residue corresponding to position 67 in SEQ ID NO: 2; F, L, Y at a residue corresponding to position 69 in SEQ ID NO: 2; A, R at a residue corresponding to position 72 in SEQ ID NO: 2; D, T, A, R at a residue corresponding to position 76 in SEQ ID NO: 2; R at a residue corresponding to position 77 in SEQ ID NO: 2; C at a residue corresponding to position 81 in SEQ ID NO: 2; Q at a residue corresponding to position 86 in SEQ ID NO: 2; R at a residue corresponding to position 87 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; M at a residue corresponding to position 98 in SEQ ID NO: 2; A at a residue corresponding to position 104 in SEQ ID NO: 2; V at a residue corresponding to position 108 in SEQ ID NO: 2; C at a residue corresponding to position 113 in SEQ ID NO: 2; I at a residue corresponding to position 116 in SEQ ID NO: 2; Y, V, F, L at a residue corresponding to position 120 in SEQ ID NO: 2; I at a residue corresponding to position 123 in SEQ ID NO: 2; S at a residue corresponding to position 124 in SEQ ID NO: 2; P, T at a residue corresponding to position 125 in SEQ ID NO: 2; R, A, N at a residue corresponding to position 126 in SEQ ID NO: 2; L at a residue corresponding to position 127 in SEQ ID NO: 2; M at a residue corresponding to position 130 in SEQ ID NO: 2; E at a residue corresponding to position 136 in SEQ ID NO: 2; M, I at a residue corresponding to position 140 in SEQ ID NO: 2; M at a residue corresponding to position 141 in SEQ ID NO: 2; Q, N at a residue corresponding to position 142 in SEQ ID NO: 2; R at a residue corresponding to position 145 in SEQ ID NO: 2; S at a residue corresponding to position 147 in SEQ ID NO: 2; V at a residue corresponding to position 148 in SEQ ID NO: 2; V at a residue corresponding to position 149 in SEQ ID NO: 2; C at a residue corresponding to position 151 in SEQ ID NO: 2; E at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 190 in SEQ ID NO: 2; E, A at a residue corresponding to position 191 in SEQ ID NO: 2; A at a residue corresponding to position 193 in SEQ ID NO: 2; S at a residue corresponding to position 195 in SEQ ID NO: 2; L, A at a residue corresponding to position 201 in SEQ ID NO: 2; T at a residue corresponding to position 205 in SEQ ID NO: 2; I at a residue corresponding to position 209 in SEQ ID NO: 2; Y at a residue corresponding to position 214 in SEQ ID NO: 2; T at a residue corresponding to position 219 in SEQ ID NO: 2; G at a residue corresponding to position 220 in SEQ ID NO: 2; K at a residue corresponding to position 221 in SEQ ID NO: 2; S at a residue corresponding to position 228 in SEQ ID NO: 2; H, K at a residue corresponding to position 230 in SEQ ID NO: 2; L at a residue corresponding to position 232 in SEQ ID NO: 2; S at a residue corresponding to position 234 in SEQ ID NO: 2; A at a residue corresponding to position 237 in SEQ ID NO: 2; M at a residue corresponding to position 238 in SEQ ID NO: 2; V at a residue corresponding to position 239 in SEQ ID NO: 2; Q, E at a residue corresponding to position 241 in SEQ ID NO: 2; C, V, T at a residue corresponding to position 242 in SEQ ID NO: 2; W, F, M at a residue corresponding to position 243 in SEQ ID NO: 2; P, Q at a residue corresponding to position 244 in SEQ ID NO: 2; I at a residue corresponding to position 246 in SEQ ID NO: 2; R at a residue corresponding to position 247 in SEQ ID NO: 2; L at a residue corresponding to position 253 in SEQ ID NO: 2; M at a residue corresponding to position 254 in SEQ ID NO: 2; S, H at a residue corresponding to position 260 in SEQ ID NO: 2; Y at a residue corresponding to position 263 in SEQ ID NO: 2; N at a residue corresponding to position 266 in SEQ ID NO: 2; M, F at a residue corresponding to position 267 in SEQ ID NO: 2; A at a residue corresponding to position 272 in SEQ ID NO: 2; I at a residue corresponding to position 273 in SEQ ID NO: 2; C at a residue corresponding to position 274 in SEQ ID NO: 2; T, A at a residue corresponding to position 278 in SEQ ID NO: 2; M at a residue corresponding to position 279 in SEQ ID NO: 2; V at a residue corresponding to position 281 in SEQ ID NO: 2; E at a residue corresponding to position 283 in SEQ ID NO: 2; M, I, L, V at a residue corresponding to position 291 in SEQ ID NO: 2; H at a residue corresponding to position 292 in SEQ ID NO: 2; V, I, M at a residue corresponding to position 295 in SEQ ID NO: 2; M, V at a residue corresponding to position 298 in SEQ ID NO: 2; Q at a residue corresponding to position 300 in SEQ ID NO: 2; Q at a residue corresponding to position 302 in SEQ ID NO: 2; A, P at a residue corresponding to position 303 in SEQ ID NO: 2; S at a residue corresponding to position 304 in SEQ ID NO: 2; V at a residue corresponding to position 305 in SEQ ID NO: 2; M at a residue corresponding to position 307 in SEQ ID NO: 2; T, A at a residue corresponding to position 311 in SEQ ID NO: 2; F at a residue corresponding to position 313 in SEQ ID NO: 2; Q, H at a residue corresponding to position 314 in SEQ ID NO: 2; A, K at a residue corresponding to position 315 in SEQ ID NO: 2; D at a residue corresponding to position 318 in SEQ ID NO: 2; R, Q at a residue corresponding to position 321 in SEQ ID NO: 2; A, K, H, Q at a residue corresponding to position 322 in SEQ ID NO: 2; F at a residue corresponding to position 330 in SEQ ID NO: 2; S at a residue corresponding to position 333 in SEQ ID NO: 2; V, I at a residue corresponding to position 334 in SEQ ID NO: 2; V, T, M at a residue corresponding to position 336 in SEQ ID NO: 2; L, V at a residue corresponding to position 337 in SEQ ID NO: 2; L, M at a residue corresponding to position 338 in SEQ ID NO: 2; G at a residue corresponding to position 340 in SEQ ID NO: 2; I at a residue corresponding to position 344 in SEQ ID NO: 2; I, L at a residue corresponding to position 348 in SEQ ID NO: 2; Y at a residue corresponding to position 349 in SEQ ID NO: 2; R, Q at a residue corresponding to position 351 in SEQ ID NO: 2; R at a residue corresponding to position 353 in SEQ ID NO: 2; K at a residue corresponding to position 354 in SEQ ID NO: 2; A at a residue corresponding to position 355 in SEQ ID NO: 2; F, I at a residue corresponding to position 357 in SEQ ID NO: 2; Q at a residue corresponding to position 358 in SEQ ID NO: 2; Q at a residue corresponding to position 359 in SEQ ID NO: 2; C, H at a residue corresponding to position 362 in SEQ ID NO: 2; V at a residue corresponding to position 363 in SEQ ID NO: 2; M at a residue corresponding to position 365 in SEQ ID NO: 2; V, I, L, M at a residue corresponding to position 367 in SEQ ID NO: 2; A at a residue corresponding to position 369 in SEQ ID NO: 2; G at a residue corresponding to position 376 in SEQ ID NO: 2; V, C at a residue corresponding to position 379 in SEQ ID NO: 2; T, C at a residue corresponding to position 382 in SEQ ID NO: 2; Y at a residue corresponding to position 383 in SEQ ID NO: 2; V, T at a residue corresponding to position 386 in SEQ ID NO: 2; R, H, N at a residue corresponding to position 387 in SEQ ID NO: 2; K at a residue corresponding to position 388 in SEQ ID NO: 2; A, P at a residue corresponding to position 389 in SEQ ID NO: 2; C, I, T, V at a residue corresponding to position 399 in SEQ ID NO: 2; Q at a residue corresponding to position 400 in SEQ ID NO: 2; L, I at a residue corresponding to position 401 in SEQ ID NO: 2; V at a residue corresponding to position 404 in SEQ ID NO: 2; P at a residue corresponding to position 407 in SEQ ID NO: 2; G at a residue corresponding to position 409 in SEQ ID NO: 2; V at a residue corresponding to position 420 in SEQ ID NO: 2; F at a residue corresponding to position 421 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; V at a residue corresponding to position 434 in SEQ ID NO: 2; Y, R, K, C, H at a residue corresponding to position 442 in SEQ ID NO: 2; L, V, M, I at a residue corresponding to position 446 in SEQ ID NO: 2; M at a residue corresponding to position 450 in SEQ ID NO: 2; P at a residue corresponding to position 452 in SEQ ID NO: 2; G at a residue corresponding to position 453 in SEQ ID NO: 2; N, D at a residue corresponding to position 456 in SEQ ID NO: 2; F, I at a residue corresponding to position 460 in SEQ ID NO: 2; Y at a residue corresponding to position 461 in SEQ ID NO: 2; L at a residue corresponding to position 465 in SEQ ID NO: 2; A at a residue corresponding to position 466 in SEQ ID NO: 2; N, S at a residue corresponding to position 473 in SEQ ID NO: 2; Y, F, V, L, I at a residue corresponding to position 476 in SEQ ID NO: 2; V, P at a residue corresponding to position 479 in SEQ ID NO: 2; S, A at a residue corresponding to position 480 in SEQ ID NO: 2; P, E at a residue corresponding to position 481 in SEQ ID NO: 2; L, E, R, A at a residue corresponding to position 485 in SEQ ID NO: 2; P at a residue corresponding to position 492 in SEQ ID NO: 2; V at a residue corresponding to position 507 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P at a residue corresponding to position 521 in SEQ ID NO: 2; M, L at a residue corresponding to position 526 in SEQ ID NO: 2; L at a residue corresponding to position 527 in SEQ ID NO: 2; R, H at a residue corresponding to position 532 in SEQ ID NO: 2; Q at a residue corresponding to position 540 in SEQ ID NO: 2; Y at a residue corresponding to position 542 in SEQ ID NO: 2; E at a residue corresponding to position 554 in SEQ ID NO: 2; S, T at a residue corresponding to position 564 in SEQ ID NO: 2; T, M, P, V, I at a residue corresponding to position 565 in SEQ ID NO: 2; A, S, Q, N at a residue corresponding to position 567 in SEQ ID NO: 2; G, D at a residue corresponding to position 568 in SEQ ID NO: 2; V at a residue corresponding to position 570 in SEQ ID NO: 2; P at a residue corresponding to position 572 in SEQ ID NO: 2; T at a residue corresponding to position 575 in SEQ ID NO: 2; N at a residue corresponding to position 584 in SEQ ID NO: 2; A at a residue corresponding to position 587 in SEQ ID NO: 2; E at a residue corresponding to position 595 in SEQ ID NO: 2; K at a residue corresponding to position 596 in SEQ ID NO: 2; or any combination thereof. It is understood that when alterations are listed herein for a certain position, the alteration can be in the alternative or in the conjunctive depending on the context. For example, the F, L, Y at a residue corresponding to position 69 in SEQ ID NO: 2, means that alterations at a residue corresponding to position 69 in SEQ ID NO: 2 can comprise F or L or Y.

In some embodiments, an engineered ACL provided herein has an amino acid sequence that is a variant of SEQ ID NO: 1 that includes a combination of alterations described in TABLE 8 or TABLE 9. Such an engineered ACL can include one or more alterations at a position described in TABLE 9 in addition to one or more alteration as described in TABLE 8. Alternatively, in some embodiments, such an engineered ACL can include one or more alteration as described in TABLE 9. Accordingly, in some embodiments, an engineered ACL provided herein has an amino acid sequence that is a variant of SEQ ID NO: 1 that includes one or more alteration as described in TABLE 9.

In some embodiments, an engineered ACL provided herein has an amino acid sequence that is a variant of SEQ ID NO: 1 that includes one or more alterations at a position described in TABLE 8, and wherein the engineered ACL further includes one or more alteration described in TABLE 9. Such alterations, in some embodiments, results in an engineered ACL having: M at a residue corresponding to position 4 in SEQ ID NO: 1; D, K, Q, S, T at a residue corresponding to position 7 in SEQ ID NO: 1; I, S at a residue corresponding to position 8 in SEQ ID NO: 1; A, K, S at a residue corresponding to position 9 in SEQ ID NO: 1; F, H, P, V, W, Y at a residue corresponding to position 10 in SEQ ID NO: 1; D, E, H, P at a residue corresponding to position 12 in SEQ ID NO: 1; C, M at a residue corresponding to position 13 in SEQ ID NO: 1; D, I, L, P at a residue corresponding to position 15 in SEQ ID NO: 1; E, H, N at a residue corresponding to position 16 in SEQ ID NO: 1; F, L, P, Q at a residue corresponding to position 19 in SEQ ID NO: 1; W at a residue corresponding to position 20 in SEQ ID NO: 1; S at a residue corresponding to position 22 in SEQ ID NO: 1; D at a residue corresponding to position 30 in SEQ ID NO: 1; A at a residue corresponding to position 31 in SEQ ID NO: 1; G, P at a residue corresponding to position 35 in SEQ ID NO: 1; H at a residue corresponding to position 36 in SEQ ID NO: 1; A at a residue corresponding to position 39 in SEQ ID NO: 1; C, F, L, M at a residue corresponding to position 40 in SEQ ID NO: 1; G at a residue corresponding to position 43 in SEQ ID NO: 1; D, H, S, T at a residue corresponding to position 44 in SEQ ID NO: 1; R, RTYTQKEL (SEQ ID NO: 15), RTYTQREL (SEQ ID NO: 16), RTYT (SEQ ID NO: 17) at a residue corresponding to position 47 in SEQ ID NO: 1; a deletion at a residue corresponding to position 48 in SEQ ID NO: 1; a deletion, Y at a residue corresponding to position 49 in SEQ ID NO: 1; a deletion, T at a residue corresponding to position 50 in SEQ ID NO: 1; a deletion, F, H, L, R, W, Y at a residue corresponding to position 51 in SEQ ID NO: 1; a deletion, K, R at a residue corresponding to position 52 in SEQ ID NO: 1; a deletion at a residue corresponding to position 53 in SEQ ID NO: 1; a deletion, L at a residue corresponding to position 54 in SEQ ID NO: 1; L at a residue corresponding to position 55 in SEQ ID NO: 1; R at a residue corresponding to position 60 in SEQ ID NO: 1; F, L at a residue corresponding to position 61 in SEQ ID NO: 1; A, G at a residue corresponding to position 64 in SEQ ID NO: 1; R at a residue corresponding to position 66 in SEQ ID NO: 1; A at a residue corresponding to position 67 in SEQ ID NO: 1; L at a residue corresponding to position 69 in SEQ ID NO: 1; G, T at a residue corresponding to position 72 in SEQ ID NO: 1; F, G, L, V at a residue corresponding to position 73 in SEQ ID NO: 1; K at a residue corresponding to position 74 in SEQ ID NO: 1; T at a residue corresponding to position 76 in SEQ ID NO: 1; V at a residue corresponding to position 78 in SEQ ID NO: 1; G at a residue corresponding to position 80 in SEQ ID NO: 1; PG at a residue corresponding to position 81 in SEQ ID NO: 1; A, E, G, N, R, T at a residue corresponding to position 82 in SEQ ID NO: 1; a deletion, K, P, R, S at a residue corresponding to position 83 in SEQ ID NO: 1; I at a residue corresponding to position 86 in SEQ ID NO: 1; I, V, VAV at a residue corresponding to position 87 in SEQ ID NO: 1; a deletion, A at a residue corresponding to position 88 in SEQ ID NO: 1; a deletion, I, L, V at a residue corresponding to position 89 in SEQ ID NO: 1; C, L, M, V, W at a residue corresponding to position 90 in SEQ ID NO: 1; A, L, M, S at a residue corresponding to position 91 in SEQ ID NO: 1; C, CIEAA (SEQ ID NO: 18), S, T, TIEAA (SEQ ID NO: 19) at a residue corresponding to position 94 in SEQ ID NO: 1; a deletion at a residue corresponding to position 95 in SEQ ID NO: 1; a deletion, E at a residue corresponding to position 96 in SEQ ID NO: 1; a deletion at a residue corresponding to position 97 in SEQ ID NO: 1; a deletion, A, L at a residue corresponding to position 98 in SEQ ID NO: 1; C, M at a residue corresponding to position 99 in SEQ ID NO: 1; S, M, L at a residue corresponding to position 100 in SEQ ID NO: 1; F, M at a residue corresponding to position 101 in SEQ ID NO: 1; H, F, L at a residue corresponding to position 102 in SEQ ID NO: 1; G at a residue corresponding to position 103 in SEQ ID NO: 1; V, L, T at a residue corresponding to position 104 in SEQ ID NO: 1; V, W, H, A, I at a residue corresponding to position 105 in SEQ ID NO: 1; S, Y, R, K at a residue corresponding to position 106 in SEQ ID NO: 1; A, C, V at a residue corresponding to position 107 in SEQ ID NO: 1; L, V, Q at a residue corresponding to position 109 in SEQ ID NO: 1; V, A, M, L, C, VTPAN (SEQ ID NO: 20) at a residue corresponding to position 111 in SEQ ID NO: 1; T, a deletion at a residue corresponding to position 112 in SEQ ID NO: 1; a deletion, L at a residue corresponding to position 113 in SEQ ID NO: 1; a deletion, A, L, V at a residue corresponding to position 114 in SEQ ID NO: 1; a deletion, N at a residue corresponding to position 115 in SEQ ID NO: 1; A, W, P at a residue corresponding to position 116 in SEQ ID NO: 1; S at a residue corresponding to position 117 in SEQ ID NO: 1; T at a residue corresponding to position 119 in SEQ ID NO: 1; R, D at a residue corresponding to position 121 in SEQ ID NO: 1; H at a residue corresponding to position 126 in SEQ ID NO: 1; M, L, MRHTG (SEQ ID NO: 21), MRITG (SEQ ID NO: 22) at a residue corresponding to position 127 in SEQ ID NO: 1; a deletion at a residue corresponding to position 128 in SEQ ID NO: 1; a deletion, I, H at a residue corresponding to position 129 in SEQ ID NO: 1; a deletion at a residue corresponding to position 130 in SEQ ID NO: 1; a deletion, G, D at a residue corresponding to position 131 in SEQ ID NO: 1; L at a residue corresponding to position 136 in SEQ ID NO: 1; T at a residue corresponding to position 137 in SEQ ID NO: 1; D, S at a residue corresponding to position 138 in SEQ ID NO: 1; E at a residue corresponding to position 139 in SEQ ID NO: 1; E, K, D at a residue corresponding to position 140 in SEQ ID NO: 1; T at a residue corresponding to position 144 in SEQ ID NO: 1; R at a residue corresponding to position 149 in SEQ ID NO: 1; I, L at a residue corresponding to position 150 in SEQ ID NO: 1; A, T, C, I at a residue corresponding to position 152 in SEQ ID NO: 1; G, S at a residue corresponding to position 153 in SEQ ID NO: 1; G at a residue corresponding to position 159 in SEQ ID NO: 1; S, H, R, SIDAF (SEQ ID NO: 23), RIESF (SEQ ID NO: 24), HFDSF (SEQ ID NO: 25), HFDAF (SEQ ID NO: 26), HIDSF (SEQ ID NO: 27) at a residue corresponding to position 162 in SEQ ID NO: 1; F, I, a deletion at a residue corresponding to position 163 in SEQ ID NO: 1; a deletion, D at a residue corresponding to position 164 in SEQ ID NO: 1; a deletion, S at a residue corresponding to position 165 in SEQ ID NO: 1; a deletion, F at a residue corresponding to position 166 in SEQ ID NO: 1; L at a residue corresponding to position 167 in SEQ ID NO: 1; G at a residue corresponding to position 170 in SEQ ID NO: 1; L, A, D at a residue corresponding to position 172 in SEQ ID NO: 1; Q at a residue corresponding to position 182 in SEQ ID NO: 1; E, G at a residue corresponding to position 189 in SEQ ID NO: 1; A at a residue corresponding to position 191 in SEQ ID NO: 1; D at a residue corresponding to position 194 in SEQ ID NO: 1; T, P, M at a residue corresponding to position 195 in SEQ ID NO: 1; T, I at a residue corresponding to position 196 in SEQ ID NO: 1; W, C at a residue corresponding to position 197 in SEQ ID NO: 1; Y, V, S, W, I, M, T at a residue corresponding to position 198 in SEQ ID NO: 1; M, L, W, P, F, C, A at a residue corresponding to position 199 in SEQ ID NO: 1; W at a residue corresponding to position 200 in SEQ ID NO: 1; F, T, N, P, Y, L at a residue corresponding to position 201 in SEQ ID NO: 1; T, H, A, Q, E, L, G at a residue corresponding to position 202 in SEQ ID NO: 1; T at a residue corresponding to position 205 in SEQ ID NO: 1; F at a residue corresponding to position 208 in SEQ ID NO: 1; P at a residue corresponding to position 209 in SEQ ID NO: 1; G at a residue corresponding to position 211 in SEQ ID NO: 1; A, C at a residue corresponding to position 212 in SEQ ID NO: 1; M, V, E at a residue corresponding to position 213 in SEQ ID NO: 1; V at a residue corresponding to position 214 in SEQ ID NO: 1; S at a residue corresponding to position 215 in SEQ ID NO: 1; Q, R, Y, H, K, G at a residue corresponding to position 217 in SEQ ID NO: 1; W, M, V at a residue corresponding to position 220 in SEQ ID NO: 1; T, A at a residue corresponding to position 221 in SEQ ID NO: 1; S, N at a residue corresponding to position 222 in SEQ ID NO: 1; A, N, I, L, H at a residue corresponding to position 223 in SEQ ID NO: 1; T, Q, H, I, N, M at a residue corresponding to position 224 in SEQ ID NO: 1; L, A, F, Q at a residue corresponding to position 225 in SEQ ID NO: 1; Y, C, T, W at a residue corresponding to position 226 in SEQ ID NO: 1; G, E, M, T, A, GKWSQD (SEQ ID NO: 28), GKWSQSY (SEQ ID NO: 29), GKWSQ (SEQ ID NO: 30), GKWSQDLK (SEQ IDNO: 31), GKWSQQLQ (SEQ ID NO: 32) at a residue corresponding to position 227 in SEQ ID NO: 1; a deletion at a residue corresponding to position 228 in SEQ ID NO: 1; a deletion, F, S, L, G, W, I, M, H at a residue corresponding to position 229 in SEQ ID NO: 1; a deletion, R, M, P, T, E, Q, A at a residue corresponding to position 230 in SEQ ID NO: 1; a deletion, Q, P, H, QDLQ (SEQ ID NO: 33) at a residue corresponding to position 231 in SEQ ID NO: 1; a deletion, W, A, Y, N, K, Q, D at a residue corresponding to position 232 in SEQ ID NO: 1; a deletion, W, R, M, Y, P at a residue corresponding to position 233 in SEQ ID NO: 1; a deletion, F, Y, T, K, R, Q, P, G, H, E at a residue corresponding to position 234 in SEQ ID NO: 1; H, S, R, K, M, Q, D, G at a residue corresponding to position 235 in SEQ ID NO: 1; P, N, H, T, D, DISAC (SEQ ID NO: 34), DVS at a residue corresponding to position 238 in SEQ ID NO: 1; V, F, R, a deletion at a residue corresponding to position 239 in SEQ ID NO: 1; a deletion, F, H, T, W, S, L, C, V at a residue corresponding to position 240 in SEQ ID NO: 1; a deletion, C, M, P, I at a residue corresponding to position 241 in SEQ ID NO: 1; a deletion, G, V, C at a residue corresponding to position 242 in SEQ ID NO: 1; L, C, M at a residue corresponding to position 243 in SEQ ID NO: 1; M, V at a residue corresponding to position 244 in SEQ ID NO: 1; W, F, L, Y at a residue corresponding to position 246 in SEQ ID NO: 1; F, C at a residue corresponding to position 247 in SEQ ID NO: 1; S, P at a residue corresponding to position 248 in SEQ ID NO: 1; A, V, M at a residue corresponding to position 249 in SEQ ID NO: 1; M, A, W at a residue corresponding to position 250 in SEQ ID NO: 1; A, H at a residue corresponding to position 251 in SEQ ID NO: 1; I, M, W, Q at a residue corresponding to position 252 in SEQ ID NO: 1; T, Q, N, I, H, V, M at a residue corresponding to position 253 in SEQ ID NO: 1; E, I, T, Y, H, V, L, P, A, M, F, S, G at a residue corresponding to position 254 in SEQ ID NO: 1; M, T, I, N, Y, C, ILLANV (SEQ ID NO: 35), ILLANVHY (SEQ ID NO: 36), ILLLNVHY (SEQ ID NO: 37), ILLLNV (SEQ ID NO: 38), ILLLNVHK (SEQ ID NO: 39), ILLHNVHK (SEQ ID NO: 40), ILLSNVHY (SEQ ID NO: 41) at a residue corresponding to position 255 in SEQ ID NO: 1; I, V, H, C, F, a deletion at a residue corresponding to position 256 in SEQ ID NO: 1; a deletion, L, T, N at a residue corresponding to position 257 in SEQ ID NO: 1; a deletion, S, L, P, M, H, T, A at a residue corresponding to position 258 in SEQ ID NO: 1; a deletion, C, A at a residue corresponding to position 259 in SEQ ID NO: 1; a deletion, F, L, M, V at a residue corresponding to position 260 in SEQ ID NO: 1; a deletion, Y, M, W, A, F, L, S at a residue corresponding to position 261 in SEQ ID NO: 1; a deletion, M, K, Y, R, G, C, L, V, S, N at a residue corresponding to position 262 in SEQ ID NO: 1; G, W, I, S, V at a residue corresponding to position 264 in SEQ ID NO: 1; R, C, H, K at a residue corresponding to position 265 in SEQ ID NO: 1; I, V, Q, C at a residue corresponding to position 266 in SEQ ID NO: 1; Y at a residue corresponding to position 267 in SEQ ID NO: 1; V, M, T, F, IMPR (SEQ ID NO: 42), EIMNR (SEQ ID NO: 43) at a residue corresponding to position 268 in SEQ ID NO: 1; M, at a residue corresponding to position 269 in SEQ ID NO: 1; a deletion, P at a residue corresponding to position 270 in SEQ ID NO: 1; a deletion, R, Q at a residue corresponding to position 271 in SEQ ID NO: 1; W at a residue corresponding to position 272 in SEQ ID NO: 1; P, PEEMLRAIQ (SEQ ID NO: 44), PEEMLQAIQ (SEQ ID NO: 45) at a residue corresponding to position 274 in SEQ ID NO: 1; D, A, E, R, K, EEMLQAIQ (SEQ ID NO: 46), EEMLRAIQ (SEQ ID NO: 47), a deletion, KEMLQAIQ (SEQ ID NO: 48) at a residue corresponding to position 275 in SEQ ID NO: 1; a deletion at a residue corresponding to position 276 in SEQ ID NO: 1; a deletion, M, Y, L, F at a residue corresponding to position 277 in SEQ ID NO: 1; a deletion at a residue corresponding to position 278 in SEQ ID NO: 1; a deletion, Q, R, QAIQ (SEQ ID NO: 49), RAIQ (SEQ ID NO: 50) at a residue corresponding to position 279 in SEQ ID NO: 1; a deletion at a residue corresponding to position 280 in SEQ ID NO: 1; a deletion, V, M, L, I at a residue corresponding to position 281 in SEQ ID NO: 1; a deletion, Q at a residue corresponding to position 282 in SEQ ID NO: 1; P, C at a residue corresponding to position 286 in SEQ ID NO: 1; T, TVFFA (SEQ ID NO: 51), TSFFA (SEQ ID NO: 52), TFFFA (SEQ ID NO: 53), TMFFA (SEQ ID NO: 54), TVFHA (SEQ ID NO: 55) at a residue corresponding to position 287 in SEQ ID NO: 1; H, F, M, Y, R, I, V, T, C, a deletion at a residue corresponding to position 288 in SEQ ID NO: 1; a deletion, M, V, A, L, F, I at a residue corresponding to position 289 in SEQ ID NO: 1; a deletion, A, W, H, C, F, V, P, L at a residue corresponding to position 290 in SEQ ID NO: 1; a deletion, A, M, G, V at a residue corresponding to position 291 in SEQ ID NO: 1; A, T at a residue corresponding to position 294 in SEQ ID NO: 1; L, a deletion at a residue corresponding to position 295 in SEQ ID NO: 1; M, L at a residue corresponding to position 296 in SEQ ID NO: 1; M, Q, I, A, L, T at a residue corresponding to position 297 in SEQ ID NO: 1; M, A, F, R, IM at a residue corresponding to position 298 in SEQ ID NO: 1; M, F at a residue corresponding to position 299 in SEQ ID NO: 1; A at a residue corresponding to position 300 in SEQ ID NO: 1; M, R, E at a residue corresponding to position 304 in SEQ ID NO: 1; D at a residue corresponding to position 306 in SEQ ID NO: 1; L at a residue corresponding to position 309 in SEQ ID NO: 1; L, D at a residue corresponding to position 310 in SEQ ID NO: 1; L at a residue corresponding to position 311 in SEQ ID NO: 1; S, SHVRSV (SEQ ID NO: 56), SHVRTV (SEQ ID NO: 57) at a residue corresponding to position 314 in SEQ ID NO: 1; S, a deletion at a residue corresponding to position 315 in SEQ ID NO: 1; a deletion, V, L, I at a residue corresponding to position 316 in SEQ ID NO: 1; a deletion, H, N, Q, T at a residue corresponding to position 317 in SEQ ID NO: 1; a deletion, Y, M, T, S, C, Q, R, H, V, W, I, L at a residue corresponding to position 318 in SEQ ID NO: 1; a deletion, C, F, V, L, M at a residue corresponding to position 319 in SEQ ID NO: 1; F, I, V, Y, T, M, W at a residue corresponding to position 320 in SEQ ID NO: 1; C at a residue corresponding to position 321 in SEQ ID NO: 1; G at a residue corresponding to position 323 in SEQ ID NO: 1; S, M at a residue corresponding to position 324 in SEQ ID NO: 1; V, I, C at a residue corresponding to position 326 in SEQ ID NO: 1; S at a residue corresponding to position 327 in SEQ ID NO: 1; H, V, R, S, RELE (SEQ ID NO: 58) at a residue corresponding to position 328 in SEQ ID NO: 1; E, a deletion, ELE at a residue corresponding to position 329 in SEQ ID NO: 1; a deletion, L, T, I at a residue corresponding to position 330 in SEQ ID NO: 1; a deletion, M, E, C, L, I at a residue corresponding to position 331 in SEQ ID NO: 1; A, W, C, M, I at a residue corresponding to position 334 in SEQ ID NO: 1; Q, R at a residue corresponding to position 335 in SEQ ID NO: 1; K at a residue corresponding to position 336 in SEQ ID NO: 1; M at a residue corresponding to position 338 in SEQ ID NO: 1; S at a residue corresponding to position 339 in SEQ ID NO: 1; H, T at a residue corresponding to position 341 in SEQ ID NO: 1; L, F at a residue corresponding to position 342 in SEQ ID NO: 1; D, H, Y at a residue corresponding to position 348 in SEQ ID NO: 1; V, C, T, F, A, W at a residue corresponding to position 351 in SEQ ID NO: 1; E, K, D at a residue corresponding to position 352 in SEQ ID NO: 1; V, L, M at a residue corresponding to position 353 in SEQ ID NO: 1; C, F, L, Y, A, K, S, I at a residue corresponding to position 354 in SEQ ID NO: 1; E at a residue corresponding to position 355 in SEQ ID NO: 1; A at a residue corresponding to position 356 in SEQ ID NO: 1; W at a residue corresponding to position 357 in SEQ ID NO: 1; M at a residue corresponding to position 359 in SEQ ID NO: 1; S at a residue corresponding to position 360 in SEQ ID NO: 1; C, S at a residue corresponding to position 362 in SEQ ID NO: 1; A, T, G at a residue corresponding to position 363 in SEQ ID NO: 1; C, S at a residue corresponding to position 364 in SEQ ID NO: 1; L, I, M, C at a residue corresponding to position 365 in SEQ ID NO: 1; S, V, T at a residue corresponding to position 366 in SEQ ID NO: 1; A, C, S, H at a residue corresponding to position 367 in SEQ ID NO: 1; H at a residue corresponding to position 368 in SEQ ID NO: 1; H, Y, I, C, L, V, W, S, T, M at a residue corresponding to position 369 in SEQ ID NO: 1; Y, R, T, S, H, G, N, Q at a residue corresponding to position 370 in SEQ ID NO: 1; W at a residue corresponding to position 371 in SEQ ID NO: 1; K, W, Q, H, S, R, N at a residue corresponding to position 372 in SEQ ID NO: 1; S, H, P, Q, E at a residue corresponding to position 373 in SEQ ID NO: 1; D, T, A, R, S, P, W, E, Q, H at a residue corresponding to position 374 in SEQ ID NO: 1; P at a residue corresponding to position 375 in SEQ ID NO: 1; T, K, E, Q, H, D at a residue corresponding to position 376 in SEQ ID NO: 1; Y, R, N at a residue corresponding to position 377 in SEQ ID NO: 1; S, Y, P, V, A at a residue corresponding to position 378 in SEQ ID NO: 1; G at a residue corresponding to position 379 in SEQ ID NO: 1; T at a residue corresponding to position 380 in SEQ ID NO: 1; V, S, I at a residue corresponding to position 381 in SEQ ID NO: 1; Q, H, T, Y, W, P at a residue corresponding to position 383 in SEQ ID NO: 1; I, P, A, M at a residue corresponding to position 384 in SEQ ID NO: 1; S, A at a residue corresponding to position 386 in SEQ ID NO: 1; G, S, H at a residue corresponding to position 387 in SEQ ID NO: 1; T, M, V, L at a residue corresponding to position 388 in SEQ ID NO: 1; R, K, E at a residue corresponding to position 389 in SEQ ID NO: 1; C, L, A at a residue corresponding to position 390 in SEQ ID NO: 1; K at a residue corresponding to position 391 in SEQ ID NO: 1; D at a residue corresponding to position 394 in SEQ ID NO: 1; D, N at a residue corresponding to position 398 in SEQ ID NO: 1; V, L, I at a residue corresponding to position 402 in SEQ ID NO: 1; L, V at a residue corresponding to position 406 in SEQ ID NO: 1; D at a residue corresponding to position 407 in SEQ ID NO: 1; N, Q, NTENAPR (SEQ ID NO: 59), NTENAPD (SEQ ID NO: 60) at a residue corresponding to position 410 in SEQ ID NO: 1; T, TENAPD (SEQ ID NO: 61), TQNAPR (SEQ ID NO: 62), a deletion, TENAPR (SEQ ID NO: 63), TQNAPN (SEQ ID NO: 64) at a residue corresponding to position 411 in SEQ ID NO: 1; a deletion, Q at a residue corresponding to position 412 in SEQ ID NO: 1; a deletion, N, NNPN (SEQ ID NO: 65) at a residue corresponding to position 413 in SEQ ID NO: 1; a deletion, A, S, N at a residue corresponding to position 414 in SEQ ID NO: 1; a deletion, P at a residue corresponding to position 415 in SEQ ID NO: 1; a deletion, D, R, N, E at a residue corresponding to position 416 in SEQ ID NO: 1; P at a residue corresponding to position 418 in SEQ ID NO: 1; G at a residue corresponding to position 419 in SEQ ID NO: 1; E at a residue corresponding to position 420 in SEQ ID NO: 1; M, V at a residue corresponding to position 424 in SEQ ID NO: 1; V, L at a residue corresponding to position 425 in SEQ ID NO: 1; M, L at a residue corresponding to position 426 in SEQ ID NO: 1; T, N, S at a residue corresponding to position 430 in SEQ ID NO: 1; I, L at a residue corresponding to position 431 in SEQ ID NO: 1; T, F at a residue corresponding to position 432 in SEQ ID NO: 1; S, K at a residue corresponding to position 433 in SEQ ID NO: 1; W at a residue corresponding to position 436 in SEQ ID NO: 1; T at a residue corresponding to position 442 in SEQ ID NO: 1; R, K, RASFA (SEQ ID NO: 66) at a residue corresponding to position 443 in SEQ ID NO: 1; a deletion at a residue corresponding to position 444 in SEQ ID NO: 1; a deletion, M, T, S, A at a residue corresponding to position 445 in SEQ ID NO: 1; a deletion at a residue corresponding to position 446 in SEQ ID NO: 1; a deletion, A at a residue corresponding to position 447 in SEQ ID NO: 1; S, G at a residue corresponding to position 455 in SEQ ID NO: 1; G, E, D at a residue corresponding to position 459 in SEQ ID NO: 1; D, G at a residue corresponding to position 461 in SEQ ID NO: 1; R, G at a residue corresponding to position 462 in SEQ ID NO: 1; L, Y at a residue corresponding to position 464 in SEQ ID NO: 1; H at a residue corresponding to position 465 in SEQ ID NO: 1; M at a residue corresponding to position 469 in SEQ ID NO: 1; C, G at a residue corresponding to position 470 in SEQ ID NO: 1; Y, K, M, W, I, R at a residue corresponding to position 471 in SEQ ID NO: 1; I, F, C, Y, L, M at a residue corresponding to position 472 in SEQ ID NO: 1; E, EDGH (SEQ ID NO: 67), EDGY (SEQ ID NO: 68) at a residue corresponding to position 474 in SEQ ID NO: 1; D, a deletion at a residue corresponding to position 475 in SEQ ID NO: 1; a deletion at a residue corresponding to position 476 in SEQ ID NO: 1; a deletion, H, F, N, E, Y at a residue corresponding to position 477 in SEQ ID NO: 1; W, I, M at a residue corresponding to position 478 in SEQ ID NO: 1; T, H, Y, F, W at a residue corresponding to position 479 in SEQ ID NO: 1; V, L at a residue corresponding to position 480 in SEQ ID NO: 1; C, I, T at a residue corresponding to position 481 in SEQ ID NO: 1; L at a residue corresponding to position 484 in SEQ ID NO: 1; D at a residue corresponding to position 486 in SEQ ID NO: 1; I at a residue corresponding to position 489 in SEQ ID NO: 1; R, C, V at a residue corresponding to position 490 in SEQ ID NO: 1; G, R, N at a residue corresponding to position 491 in SEQ ID NO: 1; F, H, Y, L at a residue corresponding to position 493 in SEQ ID NO: 1; N at a residue corresponding to position 494 in SEQ ID NO: 1; F, S, Y at a residue corresponding to position 496 in SEQ ID NO: 1; L, V, I at a residue corresponding to position 503 in SEQ ID NO: 1; R, RHPD (SEQ ID NO: 69) at a residue corresponding to position 506 in SEQ ID NO: 1; a deletion at a residue corresponding to position 507 in SEQ ID NO: 1; a deletion, P at a residue corresponding to position 508 in SEQ ID NO: 1; a deletion, D at a residue corresponding to position 509 in SEQ ID NO: 1; A, V, C at a residue corresponding to position 513 in SEQ ID NO: 1; V at a residue corresponding to position 518 in SEQ ID NO: 1; P at a residue corresponding to position 519 in SEQ ID NO: 1; E, P at a residue corresponding to position 521 in SEQ ID NO: 1; Q, A, R, M, Y, D at a residue corresponding to position 523 in SEQ ID NO: 1; S, A at a residue corresponding to position 524 in SEQ ID NO: 1; S, T, D, G, E at a residue corresponding to position 525 in SEQ ID NO: 1; P, T, V at a residue corresponding to position 527 in SEQ ID NO: 1; A, V at a residue corresponding to position 528 in SEQ ID NO: 1; V, A at a residue corresponding to position 529 in SEQ ID NO: 1; L, V at a residue corresponding to position 539 in SEQ ID NO: 1; T at a residue corresponding to position 540 in SEQ ID NO: 1; E, D at a residue corresponding to position 542 in SEQ ID NO: 1; A, K at a residue corresponding to position 546 in SEQ ID NO: 1; K, R at a residue corresponding to position 551 in SEQ ID NO: 1; S at a residue corresponding to position 553 in SEQ ID NO: 1; S, P, H at a residue corresponding to position 554 in SEQ ID NO: 1; F at a residue corresponding to position 555 in SEQ ID NO: 1; K at a residue corresponding to position 557 in SEQ ID NO: 1; P at a residue corresponding to position 559 in SEQ ID NO: 1; G at a residue corresponding to position 560 in SEQ ID NO: 1; V, a deletion at a residue corresponding to position 562 in SEQ ID NO: 1; F at a residue corresponding to position 564 in SEQ ID NO: 1; V, F, FV at a residue corresponding to position 565 in SEQ ID NO: 1; E, VE at a residue corresponding to position 566 in SEQ ID NO: 1; Q, E at a residue corresponding to position 583 in SEQ ID NO: 1; R, E at a residue corresponding to position 5 in SEQ ID NO: 1; L at a residue corresponding to position 6 in SEQ ID NO: 1; K, T at a residue corresponding to position 7 in SEQ ID NO: 1; P at a residue corresponding to position 8 in SEQ ID NO: 1; K, D, P, N, R at a residue corresponding to position 10 in SEQ ID NO: 1; A, I, V, M at a residue corresponding to position 12 in SEQ ID NO: 1; C at a residue corresponding to position 13 in SEQ ID NO: 1; P, A, Q at a residue corresponding to position 14 in SEQ ID NO: 1; E, L, S, R at a residue corresponding to position 15 in SEQ ID NO: 1; C, L, I at a residue corresponding to position 16 in SEQ ID NO: 1; D, R at a residue corresponding to position 19 in SEQ ID NO: 1; H, K, R, D at a residue corresponding to position 20 in SEQ ID NO: 1; D, E at a residue corresponding to position 22 in SEQ ID NO: 1; N at a residue corresponding to position 25 in SEQ ID NO: 1; M at a residue corresponding to position 28 in SEQ ID NO: 1; C at a residue corresponding to position 37 in SEQ ID NO: 1; R at a residue corresponding to position 39 in SEQ ID NO: 1; T at a residue corresponding to position 40 in SEQ ID NO: 1; M, L, V, I at a residue corresponding to position 41 in SEQ ID NO: 1; S at a residue corresponding to position 43 in SEQ ID NO: 1; M at a residue corresponding to position 44 in SEQ ID NO: 1; I, V at a residue corresponding to position 45 in SEQ ID NO: 1; P at a residue corresponding to position 48 in SEQ ID NO: 1; I, F, R, V, L at a residue corresponding to position 50 in SEQ ID NO: 1; R at a residue corresponding to position 51 in SEQ ID NO: 1; L at a residue corresponding to position 53 in SEQ ID NO: 1; F at a residue corresponding to position 57 in SEQ ID NO: 1; V, L, M, F at a residue corresponding to position 58 in SEQ ID NO: 1; V at a residue corresponding to position 59 in SEQ ID NO: 1; P, E at a residue corresponding to position 61 in SEQ ID NO: 1; D at a residue corresponding to position 62 in SEQ ID NO: 1; D at a residue corresponding to position 63 in SEQ ID NO: 1; G at a residue corresponding to position 66 in SEQ ID NO: 1; K at a residue corresponding to position 67 in SEQ ID NO: 1; F, Y, L at a residue corresponding to position 69 in SEQ ID NO: 1; F, W at a residue corresponding to position 71 in SEQ ID NO: 1; A, R at a residue corresponding to position 72 in SEQ ID NO: 1; F at a residue corresponding to position 74 in SEQ ID NO: 1; D at a residue corresponding to position 75 in SEQ ID NO: 1; T, D, A, R at a residue corresponding to position 76 in SEQ ID NO: 1; R at a residue corresponding to position 77 in SEQ ID NO: 1; A at a residue corresponding to position 78 in SEQ ID NO: 1; K at a residue corresponding to position 80 in SEQ ID NO: 1; C at a residue corresponding to position 81 in SEQ ID NO: 1; S, H, Q, N at a residue corresponding to position 83 in SEQ ID NO: 1; Y, H at a residue corresponding to position 84 in SEQ ID NO: 1; F at a residue corresponding to position 85 in SEQ ID NO: 1; A, Q at a residue corresponding to position 86 in SEQ ID NO: 1; R at a residue corresponding to position 87 in SEQ ID NO: 1; L at a residue corresponding to position 88 in SEQ ID NO: 1; K at a residue corresponding to position 92 in SEQ ID NO: 1; G at a residue corresponding to position 93 in SEQ ID NO: 1; R at a residue corresponding to position 94 in SEQ ID NO: 1; R at a residue corresponding to position 95 in SEQ ID NO: 1; A at a residue corresponding to position 97 in SEQ ID NO: 1; M at a residue corresponding to position 98 in SEQ ID NO: 1; L at a residue corresponding to position 99 in SEQ ID NO: 1; C at a residue corresponding to position 100 in SEQ ID NO: 1; P at a residue corresponding to position 101 in SEQ ID NO: 1; R at a residue corresponding to position 103 in SEQ ID NO: 1; L, W, A at a residue corresponding to position 104 in SEQ ID NO: 1; W, F at a residue corresponding to position 106 in SEQ ID NO: 1; V, P, I, L, W, F at a residue corresponding to position 107 in SEQ ID NO: 1; V at a residue corresponding to position 108 in SEQ ID NO: 1; V at a residue corresponding to position 109 in SEQ ID NO: 1; M, F, Y, A, D at a residue corresponding to position 110 in SEQ ID NO: 1; T, C, V at a residue corresponding to position 113 in SEQ ID NO: 1; M, W, L, G, A at a residue corresponding to position 114 in SEQ ID NO: 1; A at a residue corresponding to position 115 in SEQ ID NO: 1; I at a residue corresponding to position 116 in SEQ ID NO: 1; V, T, I at a residue corresponding to position 119 in SEQ ID NO: 1; M, F, V, A, L, W, Y at a residue corresponding to position 120 in SEQ ID NO: 1; T at a residue corresponding to position 121 in SEQ ID NO: 1; A at a residue corresponding to position 122 in SEQ ID NO: 1; M, V, I at a residue corresponding to position 123 in SEQ ID NO: 1; Y, S, N at a residue corresponding to position 124 in SEQ ID NO: 1; P, T, W at a residue corresponding to position 125 in SEQ ID NO: 1; M, N, R, A at a residue corresponding to position 126 in SEQ ID NO: 1; F, W, L at a residue corresponding to position 127 in SEQ ID NO: 1; R at a residue corresponding to position 128 in SEQ ID NO: 1; M at a residue corresponding to position 130 in SEQ ID NO: 1; E at a residue corresponding to position 131 in SEQ ID NO: 1; E, Q, K at a residue corresponding to position 136 in SEQ ID NO: 1; M at a residue corresponding to position 137 in SEQ ID NO: 1; M, C, I at a residue corresponding to position 140 in SEQ ID NO: 1; M at a residue corresponding to position 141 in SEQ ID NO: 1; N, Q at a residue corresponding to position 142 in SEQ ID NO: 1; G, R, D at a residue corresponding to position 145 in SEQ ID NO: 1; S at a residue corresponding to position 147 in SEQ ID NO: 1; L, V, A at a residue corresponding to position 148 in SEQ ID NO: 1; V, L at a residue corresponding to position 149 in SEQ ID NO: 1; V at a residue corresponding to position 150 in SEQ ID NO: 1; C at a residue corresponding to position 151 in SEQ ID NO: 1; L at a residue corresponding to position 157 in SEQ ID NO: 1; R, E at a residue corresponding to position 159 in SEQ ID NO: 1; I at a residue corresponding to position 161 in SEQ ID NO: 1; V at a residue corresponding to position 163 in SEQ ID NO: 1; A at a residue corresponding to position 164 in SEQ ID NO: 1; R at a residue corresponding to position 165 in SEQ ID NO: 1; R at a residue corresponding to position 167 in SEQ ID NO: 1; H at a residue corresponding to position 168 in SEQ ID NO: 1; I at a residue corresponding to position 169 in SEQ ID NO: 1; I, V, L at a residue corresponding to position 170 in SEQ ID NO: 1; V at a residue corresponding to position 173 in SEQ ID NO: 1; R at a residue corresponding to position 175 in SEQ ID NO: 1; D at a residue corresponding to position 176 in SEQ ID NO: 1; E at a residue corresponding to position 177 in SEQ ID NO: 1; E at a residue corresponding to position 181 in SEQ ID NO: 1; I at a residue corresponding to position 183 in SEQ ID NO: 1; S at a residue corresponding to position 185 in SEQ ID NO: 1; Y at a residue corresponding to position 186 in SEQ ID NO: 1; D at a residue corresponding to position 187 in SEQ ID NO: 1; V at a residue corresponding to position 189 in SEQ ID NO: 1; V at a residue corresponding to position 190 in SEQ ID NO: 1; A, E at a residue corresponding to position 191 in SEQ ID NO: 1; T at a residue corresponding to position 192 in SEQ ID NO: 1; A at a residue corresponding to position 193 in SEQ ID NO: 1; S at a residue corresponding to position 194 in SEQ ID NO: 1; S at a residue corresponding to position 195 in SEQ ID NO: 1; P at a residue corresponding to position 196 in SEQ ID NO: 1; A, P, L at a residue corresponding to position 201 in SEQ ID NO: 1; T at a residue corresponding to position 205 in SEQ ID NO: 1; D at a residue corresponding to position 208 in SEQ ID NO: 1; I, T, P, V at a residue corresponding to position 209 in SEQ ID NO: 1; L, T, C, M, V, A at a residue corresponding to position 211 in SEQ ID NO: 1; L, M at a residue corresponding to position 212 in SEQ ID NO: 1; I, C, V, Q, M at a residue corresponding to position 213 in SEQ ID NO: 1; Y at a residue corresponding to position 214 in SEQ ID NO: 1; S at a residue corresponding to position 215 in SEQ ID NO: 1; G at a residue corresponding to position 216 in SEQ ID NO: 1; T at a residue corresponding to position 218 in SEQ ID NO: 1; T at a residue corresponding to position 219 in SEQ ID NO: 1; G at a residue corresponding to position 220 in SEQ ID NO: 1; T, M, L, R, K, H at a residue corresponding to position 221 in SEQ ID NO: 1; S at a residue corresponding to position 222 in SEQ ID NO: 1; A at a residue corresponding to position 224 in SEQ ID NO: 1; A at a residue corresponding to position 225 in SEQ ID NO: 1; M, I, V at a residue corresponding to position 226 in SEQ ID NO: 1; L, D, H, I at a residue corresponding to position 227 in SEQ ID NO: 1; S at a residue corresponding to position 228 in SEQ ID NO: 1; H, K at a residue corresponding to position 230 in SEQ ID NO: 1; N at a residue corresponding to position 231 in SEQ ID NO: 1; L, I, V at a residue corresponding to position 232 in SEQ ID NO: 1; L, V, M, W, A, I at a residue corresponding to position 233 in SEQ ID NO: 1; S, W at a residue corresponding to position 234 in SEQ ID NO: 1; Q, S at a residue corresponding to position 235 in SEQ ID NO: 1; A, C, I, M, V at a residue corresponding to position 236 in SEQ ID NO: 1; A, M, Q, R, E at a residue corresponding to position 237 in SEQ ID NO: 1; M, S, W, N, H at a residue corresponding to position 238 in SEQ ID NO: 1; F, M, V, C, L at a residue corresponding to position 239 in SEQ ID NO: 1; A, R at a residue corresponding to position 240 in SEQ ID NO: 1; A, H, E, Q at a residue corresponding to position 241 in SEQ ID NO: 1; V, C, T, W at a residue corresponding to position 242 in SEQ ID NO: 1; I, F, M, W, V at a residue corresponding to position 243 in SEQ ID NO: 1; E, P, Q, H, G at a residue corresponding to position 244 in SEQ ID NO: 1; M, L, H, W, Y at a residue corresponding to position 245 in SEQ ID NO: 1; I, T, M at a residue corresponding to position 246 in SEQ ID NO: 1; R, P, E at a residue corresponding to position 247 in SEQ ID NO: 1; E at a residue corresponding to position 251 in SEQ ID NO: 1; H, V at a residue corresponding to position 252 in SEQ ID NO: 1; Y, M, F, L, V at a residue corresponding to position 253 in SEQ ID NO: 1; I, F, V, M at a residue corresponding to position 254 in SEQ ID NO: 1; A, T, N, L, I, G, H, C, V, S at a residue corresponding to position 255 in SEQ ID NO: 1; F, V, I, C, Y at a residue corresponding to position 256 in SEQ ID NO: 1; M at a residue corresponding to position 257 in SEQ ID NO: 1; L, F at a residue corresponding to position 259 in SEQ ID NO: 1; H, S, Y, F at a residue corresponding to position 260 in SEQ ID NO: 1; C, T, A, I, M at a residue corresponding to position 262 in SEQ ID NO: 1; A, Y, H at a residue corresponding to position 263 in SEQ ID NO: 1; A at a residue corresponding to position 264 in SEQ ID NO: 1; M, L, W, F, C at a residue corresponding to position 265 in SEQ ID NO: 1; N, T, M, V at a residue corresponding to position 266 in SEQ ID NO: 1; I, G, M, W, F, L, V, C, A at a residue corresponding to position 267 in SEQ ID NO: 1; M, A, C, G, V at a residue corresponding to position 268 in SEQ ID NO: 1; L, M, I, V at a residue corresponding to position 269 in SEQ ID NO: 1; M, H, Y, T at a residue corresponding to position 270 in SEQ ID NO: 1; A, P, C, T, L at a residue corresponding to position 271 in SEQ ID NO: 1; C, V, P, A, T at a residue corresponding to position 272 in SEQ ID NO: 1; M, V, F, I at a residue corresponding to position 273 in SEQ ID NO: 1; Y, R, V, M, C, H, W at a residue corresponding to position 274 in SEQ ID NO: 1; G, C, S, H at a residue corresponding to position 275 in SEQ ID NO: 1; A, C at a residue corresponding to position 277 in SEQ ID NO: 1; C, T, A, H at a residue corresponding to position 278 in SEQ ID NO: 1; V, M, I at a residue corresponding to position 279 in SEQ ID NO: 1; F, L, V, M at a residue corresponding to position 281 in SEQ ID NO: 1; P, M, E, A, H at a residue corresponding to position 282 in SEQ ID NO: 1; E, D, P, R at a residue corresponding to position 283 in SEQ ID NO: 1; F, R, W at a residue corresponding to position 284 in SEQ ID NO: 1; R, F, W, D at a residue corresponding to position 285 in SEQ ID NO: 1; D, F at a residue corresponding to position 286 in SEQ ID NO: 1; P, A, V at a residue corresponding to position 287 in SEQ ID NO: 1; A at a residue corresponding to position 290 in SEQ ID NO: 1; M, V, L, P, I at a residue corresponding to position 291 in SEQ ID NO: 1; H at a residue corresponding to position 292 in SEQ ID NO: 1; I, L, A at a residue corresponding to position 294 in SEQ ID NO: 1; W, M, V, I at a residue corresponding to position 295 in SEQ ID NO: 1; D, Q at a residue corresponding to position 296 in SEQ ID NO: 1; M, V, L at a residue corresponding to position 298 in SEQ ID NO: 1; A at a residue corresponding to position 299 in SEQ ID NO: 1; Q at a residue corresponding to position 300 in SEQ ID NO: 1; Q at a residue corresponding to position 302 in SEQ ID NO: 1; V, I, A, P, C at a residue corresponding to position 303 in SEQ ID NO: 1; S at a residue corresponding to position 304 in SEQ ID NO: 1; W, Y, V, I, F, H, M at a residue corresponding to position 305 in SEQ ID NO: 1; V, Y, I, M, L, F at a residue corresponding to position 306 in SEQ ID NO: 1; P, W, Y, H, M at a residue corresponding to position 307 in SEQ ID NO: 1; T, A, S, L, G, V, M at a residue corresponding to position 308 in SEQ ID NO: 1; A, R, P, T at a residue corresponding to position 311 in SEQ ID NO: 1; I, M, V, F at a residue corresponding to position 312 in SEQ ID NO: 1; L, F, W at a residue corresponding to position 313 in SEQ ID NO: 1; Q, E, H at a residue corresponding to position 314 in SEQ ID NO: 1; R, A, K, L at a residue corresponding to position 315 in SEQ ID NO: 1; W, M, I, F at a residue corresponding to position 316 in SEQ ID NO: 1; H, D at a residue corresponding to position 318 in SEQ ID NO: 1; R, Q at a residue corresponding to position 321 in SEQ ID NO: 1; Q, K, A, H at a residue corresponding to position 322 in SEQ ID NO: 1; L, F at a residue corresponding to position 330 in SEQ ID NO: 1; G at a residue corresponding to position 331 in SEQ ID NO: 1; S at a residue corresponding to position 333 in SEQ ID NO: 1; I, M, V at a residue corresponding to position 334 in SEQ ID NO: 1; R, K at a residue corresponding to position 335 in SEQ ID NO: 1; T, I, M, Y, V, F, W at a residue corresponding to position 336 in SEQ ID NO: 1; L, M, F, A, V at a residue corresponding to position 337 in SEQ ID NO: 1; W, L, M, V at a residue corresponding to position 338 in SEQ ID NO: 1; F, T, S, C at a residue corresponding to position 339 in SEQ ID NO: 1; G at a residue corresponding to position 340 in SEQ ID NO: 1; A, S at a residue corresponding to position 341 in SEQ ID NO: 1; S at a residue corresponding to position 342 in SEQ ID NO: 1; P, A at a residue corresponding to position 343 in SEQ ID NO: 1; M, I, V, C at a residue corresponding to position 344 in SEQ ID NO: 1; S at a residue corresponding to position 345 in SEQ ID NO: 1; V, A, P, E at a residue corresponding to position 346 in SEQ ID NO: 1; E at a residue corresponding to position 347 in SEQ ID NO: 1; T, I, L at a residue corresponding to position 348 in SEQ ID NO: 1; M, F, L, A, I, Y at a residue corresponding to position 349 in SEQ ID NO: 1; F, R, Q, W at a residue corresponding to position 351 in SEQ ID NO: 1; I, M, Y at a residue corresponding to position 352 in SEQ ID NO: 1; R at a residue corresponding to position 353 in SEQ ID NO: 1; K at a residue corresponding to position 354 in SEQ ID NO: 1; A, R at a residue corresponding to position 355 in SEQ ID NO: 1; W, G, V, L, P, I, F, A at a residue corresponding to position 357 in SEQ ID NO: 1; Q at a residue corresponding to position 358 in SEQ ID NO: 1; H, Q at a residue corresponding to position 359 in SEQ ID NO: 1; F at a residue corresponding to position 361 in SEQ ID NO: 1; H, L, C at a residue corresponding to position 362 in SEQ ID NO: 1; D, V, I, G at a residue corresponding to position 363 in SEQ ID NO: 1; M, V, I at a residue corresponding to position 365 in SEQ ID NO: 1; I, V, L, F at a residue corresponding to position 366 in SEQ ID NO: 1; T, C, M, I, V, L at a residue corresponding to position 367 in SEQ ID NO: 1; N, Q, E, T at a residue corresponding to position 368 in SEQ ID NO: 1; G, A at a residue corresponding to position 369 in SEQ ID NO: 1; Y, F at a residue corresponding to position 370 in SEQ ID NO: 1; L, Q at a residue corresponding to position 372 in SEQ ID NO: 1; S at a residue corresponding to position 373 in SEQ ID NO: 1; A, C at a residue corresponding to position 375 in SEQ ID NO: 1; T, S, C, G at a residue corresponding to position 376 in SEQ ID NO: 1; G, M at a residue corresponding to position 377 in SEQ ID NO: 1; C, T, V at a residue corresponding to position 378 in SEQ ID NO: 1; C, V, I, L, A at a residue corresponding to position 379 in SEQ ID NO: 1; F, C, H, S, M, T, V at a residue corresponding to position 380 in SEQ ID NO: 1; N, F, L, C, V at a residue corresponding to position 381 in SEQ ID NO: 1; L, N, C, T, P at a residue corresponding to position 382 in SEQ ID NO: 1; D, N, R, P, Y at a residue corresponding to position 383 in SEQ ID NO: 1; P, N, D, W at a residue corresponding to position 384 in SEQ ID NO: 1; G, D at a residue corresponding to position 385 in SEQ ID NO: 1; D, V, L, T at a residue corresponding to position 386 in SEQ ID NO: 1; D, R, N, H, E at a residue corresponding to position 387 in SEQ ID NO: 1; K, H at a residue corresponding to position 388 in SEQ ID NO: 1; A, P, L at a residue corresponding to position 389 in SEQ ID NO: 1; S, T at a residue corresponding to position 391 in SEQ ID NO: 1; V, C at a residue corresponding to position 392 in SEQ ID NO: 1; R, K, I, Q, L at a residue corresponding to position 394 in SEQ ID NO: 1; A at a residue corresponding to position 395 in SEQ ID NO: 1; F, I, M, V at a residue corresponding to position 396 in SEQ ID NO: 1; D, H, N at a residue corresponding to position 398 in SEQ ID NO: 1; M, V, T, C, I at a residue corresponding to position 399 in SEQ ID NO: 1; D, Q at a residue corresponding to position 400 in SEQ ID NO: 1; M, I, C, L, A at a residue corresponding to position 401 in SEQ ID NO: 1; R at a residue corresponding to position 402 in SEQ ID NO: 1; V at a residue corresponding to position 404 in SEQ ID NO: 1; D at a residue corresponding to position 405 in SEQ ID NO: 1; E at a residue corresponding to position 406 in SEQ ID NO: 1; P at a residue corresponding to position 407 in SEQ ID NO: 1; Q, E, G at a residue corresponding to position 409 in SEQ ID NO: 1; D, S, V, G, N at a residue corresponding to position 411 in SEQ ID NO: 1; I, V at a residue corresponding to position 413 in SEQ ID NO: 1; W, L, V, C, Q at a residue corresponding to position 414 in SEQ ID NO: 1; I at a residue corresponding to position 415 in SEQ ID NO: 1; R at a residue corresponding to position 416 in SEQ ID NO: 1; S at a residue corresponding to position 417 in SEQ ID NO: 1; D at a residue corresponding to position 418 in SEQ ID NO: 1; H, S, Q, M at a residue corresponding to position 419 in SEQ ID NO: 1; V at a residue corresponding to position 420 in SEQ ID NO: 1; T, F at a residue corresponding to position 421 in SEQ ID NO: 1; H, L at a residue corresponding to position 425 in SEQ ID NO: 1; A at a residue corresponding to position 430 in SEQ ID NO: 1; K at a residue corresponding to position 432 in SEQ ID NO: 1; R, D at a residue corresponding to position 433 in SEQ ID NO: 1; T, V at a residue corresponding to position 434 in SEQ ID NO: 1; I, M, L at a residue corresponding to position 435 in SEQ ID NO: 1; F, M, Y at a residue corresponding to position 441 in SEQ ID NO: 1; K, H, Y, R, C at a residue corresponding to position 442 in SEQ ID NO: 1; S at a residue corresponding to position 443 in SEQ ID NO: 1; M, I, L, V at a residue corresponding to position 446 in SEQ ID NO: 1; A, G at a residue corresponding to position 447 in SEQ ID NO: 1; I at a residue corresponding to position 449 in SEQ ID NO: 1; D, M at a residue corresponding to position 450 in SEQ ID NO: 1; E at a residue corresponding to position 451 in SEQ ID NO: 1; P at a residue corresponding to position 452 in SEQ ID NO: 1; G at a residue corresponding to position 453 in SEQ ID NO: 1; E, G, R at a residue corresponding to position 455 in SEQ ID NO: 1; D, E, Q, N at a residue corresponding to position 456 in SEQ ID NO: 1; N, F, Y, W at a residue corresponding to position 458 in SEQ ID NO: 1; Y at a residue corresponding to position 459 in SEQ ID NO: 1; Y, F, I, R, W at a residue corresponding to position 460 in SEQ ID NO: 1; I, L, Y at a residue corresponding to position 461 in SEQ ID NO: 1; I, T, V at a residue corresponding to position 462 in SEQ ID NO: 1; D at a residue corresponding to position 463 in SEQ ID NO: 1; D at a residue corresponding to position 464 in SEQ ID NO: 1; L at a residue corresponding to position 465 in SEQ ID NO: 1; D, K, A at a residue corresponding to position 466 in SEQ ID NO: 1; K at a residue corresponding to position 467 in SEQ ID NO: 1; M at a residue corresponding to position 468 in SEQ ID NO: 1; L at a residue corresponding to position 469 in SEQ ID NO: 1; V at a residue corresponding to position 470 in SEQ ID NO: 1; T, V at a residue corresponding to position 471 in SEQ ID NO: 1; S, N at a residue corresponding to position 473 in SEQ ID NO: 1; E at a residue corresponding to position 474 in SEQ ID NO: 1; N, Y, L, F, V, I at a residue corresponding to position 476 in SEQ ID NO: 1; T, L at a residue corresponding to position 478 in SEQ ID NO: 1; P, V at a residue corresponding to position 479 in SEQ ID NO: 1; L, I, S, P, A, V at a residue corresponding to position 480 in SEQ ID NO: 1; P, E at a residue corresponding to position 481 in SEQ ID NO: 1; E at a residue corresponding to position 483 in SEQ ID NO: 1; A, L, E, R at a residue corresponding to position 485 in SEQ ID NO: 1; C at a residue corresponding to position 490 in SEQ ID NO: 1; P at a residue corresponding to position 492 in SEQ ID NO: 1; F at a residue corresponding to position 493 in SEQ ID NO: 1; H at a residue corresponding to position 496 in SEQ ID NO: 1; A at a residue corresponding to position 497 in SEQ ID NO: 1; C at a residue corresponding to position 498 in SEQ ID NO: 1; P at a residue corresponding to position 502 in SEQ ID NO: 1; H at a residue corresponding to position 504 in SEQ ID NO: 1; Y, V, W at a residue corresponding to position 507 in SEQ ID NO: 1; V at a residue corresponding to position 508 in SEQ ID NO: 1; A at a residue corresponding to position 510 in SEQ ID NO: 1; V, L at a residue corresponding to position 511 in SEQ ID NO: 1; I at a residue corresponding to position 512 in SEQ ID NO: 1; V at a residue corresponding to position 513 in SEQ ID NO: 1; N at a residue corresponding to position 514 in SEQ ID NO: 1; D, S at a residue corresponding to position 515 in SEQ ID NO: 1; A at a residue corresponding to position 516 in SEQ ID NO: 1; I at a residue corresponding to position 518 in SEQ ID NO: 1; T at a residue corresponding to position 519 in SEQ ID NO: 1; P, W at a residue corresponding to position 521 in SEQ ID NO: 1; L, M at a residue corresponding to position 526 in SEQ ID NO: 1; L at a residue corresponding to position 527 in SEQ ID NO: 1; R, H at a residue corresponding to position 532 in SEQ ID NO: 1; L at a residue corresponding to position 535 in SEQ ID NO: 1; Q at a residue corresponding to position 540 in SEQ ID NO: 1; Y, W at a residue corresponding to position 542 in SEQ ID NO: 1; N at a residue corresponding to position 546 in SEQ ID NO: 1; Q at a residue corresponding to position 552 in SEQ ID NO: 1; E at a residue corresponding to position 554 in SEQ ID NO: 1; F at a residue corresponding to position 558 in SEQ ID NO: 1; D at a residue corresponding to position 560 in SEQ ID NO: 1; D at a residue corresponding to position 561 in SEQ ID NO: 1; F, L, W at a residue corresponding to position 563 in SEQ ID NO: 1; D, K, S, L, T, R at a residue corresponding to position 564 in SEQ ID NO: 1; M, I, V, P, T at a residue corresponding to position 565 in SEQ ID NO: 1; A, E, D, P at a residue corresponding to position 566 in SEQ ID NO: 1; K, Q, T, N, S, A at a residue corresponding to position 567 in SEQ ID NO: 1; G, N, D at a residue corresponding to position 568 in SEQ ID NO: 1; M, K at a residue corresponding to position 569 in SEQ ID NO: 1; V, L at a residue corresponding to position 570 in SEQ ID NO: 1; R, P at a residue corresponding to position 572 in SEQ ID NO: 1; T, S at a residue corresponding to position 573 in SEQ ID NO: 1; M, L at a residue corresponding to position 574 in SEQ ID NO: 1; A, Y, K, T at a residue corresponding to position 575 in SEQ ID NO: 1; Y, A, L at a residue corresponding to position 577 in SEQ ID NO: 1; Q, A at a residue corresponding to position 578 in SEQ ID NO: 1; D at a residue corresponding to position 583 in SEQ ID NO: 1; N at a residue corresponding to position 584 in SEQ ID NO: 1; H, L at a residue corresponding to position 585 in SEQ ID NO: 1; A at a residue corresponding to position 587 in SEQ ID NO: 1; Q at a residue corresponding to position 588 in SEQ ID NO: 1; Q, E at a residue corresponding to position 591 in SEQ ID NO: 1; A at a residue corresponding to position 593 in SEQ ID NO: 1; E at a residue corresponding to position 595 in SEQ ID NO: 1; K at a residue corresponding to position 596 in SEQ ID NO: 1; D at a residue corresponding to position 598 in SEQ ID NO: 1; S at a residue corresponding to position 599 in SEQ ID NO: 1; L at a residue corresponding to position 601 in SEQ ID NO: 1; or any combination thereof. It is understood that when listed herein, alterations listed for a certain position can be in the alternative or in the conjunctive depending on the context. For example, the V, F, FV alterations at a residue corresponding to position 565 in SEQ ID NO: 1, indicates that alterations at a residue corresponding to position 565 in SEQ ID NO: 1 can comprise V or F or FV, wherein FV is F and V.

In some embodiments, an engineered ACL provided herein has an amino acid sequence that is a variant of SEQ ID NO: 2 that includes one or more alteration as described in TABLE 9 or TABLE 8. Such an engineered ACL can include one or more alterations at a position described in TABLE 8 in addition to one or more alteration as described in TABLE 9. Alternatively, in some embodiments, such an engineered ACL can include one or more alteration as described in TABLE 8. Accordingly, in some embodiments, an engineered ACL provided herein has an amino acid sequence that is a variant of SEQ ID NO: 2 that includes one or more alteration as described in TABLE 8.

In some embodiments, an engineered ACL provided herein has an amino acid sequence that is a variant of SEQ ID NO: 2 that includes one or more alterations at a position described in TABLE 9, and wherein the engineered ACL further includes one or more alteration described in TABLE 8. Such alterations, in some embodiments, results in an engineered ACL having: M at a residue corresponding to position 4 in SEQ ID NO: 2; D, K, Q, S, T at a residue corresponding to position 7 in SEQ ID NO: 2; I, S at a residue corresponding to position 8 in SEQ ID NO: 2; A, K, S at a residue corresponding to position 9 in SEQ ID NO: 2; F, H, P, V, W, Y at a residue corresponding to position 10 in SEQ ID NO: 2; D, E, H, P at a residue corresponding to position 12 in SEQ ID NO: 2; C, M at a residue corresponding to position 13 in SEQ ID NO: 2; D, I, L, P at a residue corresponding to position 15 in SEQ ID NO: 2; E, H, N at a residue corresponding to position 16 in SEQ ID NO: 2; F, L, P, Q at a residue corresponding to position 19 in SEQ ID NO: 2; W at a residue corresponding to position 20 in SEQ ID NO: 2; S at a residue corresponding to position 22 in SEQ ID NO: 2; D at a residue corresponding to position 30 in SEQ ID NO: 2; A at a residue corresponding to position 31 in SEQ ID NO: 2; G, P at a residue corresponding to position 35 in SEQ ID NO: 2; H at a residue corresponding to position 36 in SEQ ID NO: 2; A at a residue corresponding to position 39 in SEQ ID NO: 2; C, F, L, M at a residue corresponding to position 40 in SEQ ID NO: 2; G at a residue corresponding to position 43 in SEQ ID NO: 2; D, H, S, T at a residue corresponding to position 44 in SEQ ID NO: 2; R, RTYTQKEL (SEQ ID NO: 15), RTYTQREL (SEQ ID NO: 16), RTYT (SEQ ID NO: 17) at a residue corresponding to position 47 in SEQ ID NO: 2; a deletion at a residue corresponding to position 48 in SEQ ID NO: 2; a deletion, Y at a residue corresponding to position 49 in SEQ ID NO: 2; a deletion, T at a residue corresponding to position 50 in SEQ ID NO: 2; a deletion, F, H, L, R, W, Y at a residue corresponding to position 51 in SEQ ID NO: 2; a deletion, K, R at a residue corresponding to position 52 in SEQ ID NO: 2; a deletion at a residue corresponding to position 53 in SEQ ID NO: 2; a deletion, L at a residue corresponding to position 54 in SEQ ID NO: 2; L at a residue corresponding to position 55 in SEQ ID NO: 2; R at a residue corresponding to position 60 in SEQ ID NO: 2; F, L at a residue corresponding to position 61 in SEQ ID NO: 2; A, G at a residue corresponding to position 64 in SEQ ID NO: 2; R at a residue corresponding to position 66 in SEQ ID NO: 2; A at a residue corresponding to position 67 in SEQ ID NO: 2; L at a residue corresponding to position 69 in SEQ ID NO: 2; G, T at a residue corresponding to position 72 in SEQ ID NO: 2; F, G, L, V at a residue corresponding to position 73 in SEQ ID NO: 2; K at a residue corresponding to position 74 in SEQ ID NO: 2; T at a residue corresponding to position 76 in SEQ ID NO: 2; V at a residue corresponding to position 78 in SEQ ID NO: 2; G at a residue corresponding to position 80 in SEQ ID NO: 2; PG at a residue corresponding to position 81 in SEQ ID NO: 2; A, E, G, N, R, T at a residue corresponding to position 82 in SEQ ID NO: 2; a deletion, K, P, R, S at a residue corresponding to position 83 in SEQ ID NO: 2; I at a residue corresponding to position 86 in SEQ ID NO: 2; I, V, VAV at a residue corresponding to position 87 in SEQ ID NO: 2; a deletion, A at a residue corresponding to position 88 in SEQ ID NO: 2; a deletion, I, L, V at a residue corresponding to position 89 in SEQ ID NO: 2; C, L, M, V, W at a residue corresponding to position 90 in SEQ ID NO: 2; A, L, M, S at a residue corresponding to position 91 in SEQ ID NO: 2; C, CIEAA (SEQ ID NO: 18), S, T, TIEAA (SEQ ID NO: 19) at a residue corresponding to position 94 in SEQ ID NO: 2; a deletion at a residue corresponding to position 95 in SEQ ID NO: 2; a deletion, E at a residue corresponding to position 96 in SEQ ID NO: 2; a deletion at a residue corresponding to position 97 in SEQ ID NO: 2; a deletion, A, L at a residue corresponding to position 98 in SEQ ID NO: 2; C, M at a residue corresponding to position 99 in SEQ ID NO: 2; S, M, L at a residue corresponding to position 100 in SEQ ID NO: 2; F, M at a residue corresponding to position 101 in SEQ ID NO: 2; H, F, L at a residue corresponding to position 102 in SEQ ID NO: 2; G at a residue corresponding to position 103 in SEQ ID NO: 2; V, L, T at a residue corresponding to position 104 in SEQ ID NO: 2; V, W, H, A, I at a residue corresponding to position 105 in SEQ ID NO: 2; S, Y, R, K at a residue corresponding to position 106 in SEQ ID NO: 2; A, C, V at a residue corresponding to position 107 in SEQ ID NO: 2; L, V, Q at a residue corresponding to position 109 in SEQ ID NO: 2; V, A, M, L, C, VTPAN (SEQ ID NO: 20) at a residue corresponding to position 111 in SEQ ID NO: 2; T, a deletion at a residue corresponding to position 112 in SEQ ID NO: 2; a deletion, L at a residue corresponding to position 113 in SEQ ID NO: 2; a deletion, A, L, V at a residue corresponding to position 114 in SEQ ID NO: 2; a deletion, N at a residue corresponding to position 115 in SEQ ID NO: 2; A, W, P at a residue corresponding to position 116 in SEQ ID NO: 2; S at a residue corresponding to position 117 in SEQ ID NO: 2; T at a residue corresponding to position 119 in SEQ ID NO: 2; R, D at a residue corresponding to position 121 in SEQ ID NO: 2; H at a residue corresponding to position 126 in SEQ ID NO: 2; M, L, MRHTG (SEQ ID NO: 21), MRITG (SEQ ID NO: 22) at a residue corresponding to position 127 in SEQ ID NO: 2; a deletion at a residue corresponding to position 128 in SEQ ID NO: 2; a deletion, I, H at a residue corresponding to position 129 in SEQ ID NO: 2; a deletion at a residue corresponding to position 130 in SEQ ID NO: 2; a deletion, G, D at a residue corresponding to position 131 in SEQ ID NO: 2; L at a residue corresponding to position 136 in SEQ ID NO: 2; T at a residue corresponding to position 137 in SEQ ID NO: 2; D, S at a residue corresponding to position 138 in SEQ ID NO: 2; E at a residue corresponding to position 139 in SEQ ID NO: 2; E, K, D at a residue corresponding to position 140 in SEQ ID NO: 2; T at a residue corresponding to position 144 in SEQ ID NO: 2; R at a residue corresponding to position 149 in SEQ ID NO: 2; I, L at a residue corresponding to position 150 in SEQ ID NO: 2; A, T, C, I at a residue corresponding to position 152 in SEQ ID NO: 2; G, S at a residue corresponding to position 153 in SEQ ID NO: 2; G at a residue corresponding to position 159 in SEQ ID NO: 2; S, H, R, SIDAF (SEQ ID NO: 23), RIESF (SEQ ID NO: 24), HFDSF (SEQ ID NO: 25), HFDAF (SEQ ID NO: 26), HIDSF (SEQ ID NO: 27) at a residue corresponding to position 162 in SEQ ID NO: 2; F, I, a deletion at a residue corresponding to position 163 in SEQ ID NO: 2; a deletion, D at a residue corresponding to position 164 in SEQ ID NO: 2; a deletion, S at a residue corresponding to position 165 in SEQ ID NO: 2; a deletion, F at a residue corresponding to position 166 in SEQ ID NO: 2; L at a residue corresponding to position 167 in SEQ ID NO: 2; G at a residue corresponding to position 170 in SEQ ID NO: 2; L, A, D at a residue corresponding to position 172 in SEQ ID NO: 2; Q at a residue corresponding to position 182 in SEQ ID NO: 2; E, G at a residue corresponding to position 189 in SEQ ID NO: 2; A at a residue corresponding to position 191 in SEQ ID NO: 2; D at a residue corresponding to position 194 in SEQ ID NO: 2; T, P, M at a residue corresponding to position 195 in SEQ ID NO: 2; T, I at a residue corresponding to position 196 in SEQ ID NO: 2; W, C at a residue corresponding to position 197 in SEQ ID NO: 2; Y, V, S, W, I, M, T at a residue corresponding to position 198 in SEQ ID NO: 2; M, L, W, P, F, C, A at a residue corresponding to position 199 in SEQ ID NO: 2; W at a residue corresponding to position 200 in SEQ ID NO: 2; F, T, N, P, Y, L at a residue corresponding to position 201 in SEQ ID NO: 2; T, H, A, Q, E, L, G at a residue corresponding to position 202 in SEQ ID NO: 2; T at a residue corresponding to position 205 in SEQ ID NO: 2; F at a residue corresponding to position 208 in SEQ ID NO: 2; P at a residue corresponding to position 209 in SEQ ID NO: 2; G at a residue corresponding to position 211 in SEQ ID NO: 2; A, C at a residue corresponding to position 212 in SEQ ID NO: 2; M, V, E at a residue corresponding to position 213 in SEQ ID NO: 2; V at a residue corresponding to position 214 in SEQ ID NO: 2; S at a residue corresponding to position 215 in SEQ ID NO: 2; Q, R, Y, H, K, G at a residue corresponding to position 217 in SEQ ID NO: 2; W, M, V at a residue corresponding to position 220 in SEQ ID NO: 2; T, A at a residue corresponding to position 221 in SEQ ID NO: 2; S, N at a residue corresponding to position 222 in SEQ ID NO: 2; A, N, I, L, H at a residue corresponding to position 223 in SEQ ID NO: 2; T, Q, H, I, N, M at a residue corresponding to position 224 in SEQ ID NO: 2; L, A, F, Q at a residue corresponding to position 225 in SEQ ID NO: 2; Y, C, T, W at a residue corresponding to position 226 in SEQ ID NO: 2; G, E, M, T, A, GKWSQD (SEQ ID NO: 28), GKWSQSY (SEQ ID NO: 29), GKWSQ (SEQ ID NO: 30), GKWSQDLK (SEQ IDNO: 31), GKWSQQLQ (SEQ ID NO: 32) at a residue corresponding to position 227 in SEQ ID NO: 2; a deletion at a residue corresponding to position 228 in SEQ ID NO: 2; a deletion, F, S, L, G, W, I, M, H at a residue corresponding to position 229 in SEQ ID NO: 2; a deletion, R, M, P, T, E, Q, A at a residue corresponding to position 230 in SEQ ID NO: 2; a deletion, Q, P, H, QDLQ (SEQ ID NO: 33) at a residue corresponding to position 231 in SEQ ID NO: 2; a deletion, W, A, Y, N, K, Q, D at a residue corresponding to position 232 in SEQ ID NO: 2; a deletion, W, R, M, Y, P at a residue corresponding to position 233 in SEQ ID NO: 2; a deletion, F, Y, T, K, R, Q, P, G, H, E at a residue corresponding to position 234 in SEQ ID NO: 2; H, S, R, K, M, Q, D, G at a residue corresponding to position 235 in SEQ ID NO: 2; P, N, H, T, D, DISAC (SEQ ID NO: 34), DVS at a residue corresponding to position 238 in SEQ ID NO: 2; V, F, R, a deletion at a residue corresponding to position 239 in SEQ ID NO: 2; a deletion, F, H, T, W, S, L, C, V at a residue corresponding to position 240 in SEQ ID NO: 2; a deletion, C, M, P, I at a residue corresponding to position 241 in SEQ ID NO: 2; a deletion, G, V, C at a residue corresponding to position 242 in SEQ ID NO: 2; L, C, M at a residue corresponding to position 243 in SEQ ID NO: 2; M, V at a residue corresponding to position 244 in SEQ ID NO: 2; W, F, L, Y at a residue corresponding to position 246 in SEQ ID NO: 2; F, C at a residue corresponding to position 247 in SEQ ID NO: 2; S, P at a residue corresponding to position 248 in SEQ ID NO: 2; A, V, M at a residue corresponding to position 249 in SEQ ID NO: 2; M, A, W at a residue corresponding to position 250 in SEQ ID NO: 2; A, H at a residue corresponding to position 251 in SEQ ID NO: 2; I, M, W, Q at a residue corresponding to position 252 in SEQ ID NO: 2; T, Q, N, I, H, V, M at a residue corresponding to position 253 in SEQ ID NO: 2; E, I, T, Y, H, V, L, P, A, M, F, S, G at a residue corresponding to position 254 in SEQ ID NO: 2; M, T, I, N, Y, C, ILLANV (SEQ ID NO: 35), ILLANVHY (SEQ ID NO: 36), ILLLNVHY (SEQ ID NO: 37), ILLLNV (SEQ ID NO: 38), ILLLNVHK (SEQ ID NO: 39), ILLHNVHK (SEQ ID NO: 40), ILLSNVHY (SEQ ID NO: 41) at a residue corresponding to position 255 in SEQ ID NO: 2; I, V, H, C, F, a deletion at a residue corresponding to position 256 in SEQ ID NO: 2; a deletion, L, T, N at a residue corresponding to position 257 in SEQ ID NO: 2; a deletion, S, L, P, M, H, T, A at a residue corresponding to position 258 in SEQ ID NO: 2; a deletion, C, A at a residue corresponding to position 259 in SEQ ID NO: 2; a deletion, F, L, M, V at a residue corresponding to position 260 in SEQ ID NO: 2; a deletion, Y, M, W, A, F, L, S at a residue corresponding to position 261 in SEQ ID NO: 2; a deletion, M, K, Y, R, G, C, L, V, S, N at a residue corresponding to position 262 in SEQ ID NO: 2; G, W, I, S, V at a residue corresponding to position 264 in SEQ ID NO: 2; R, C, H, K at a residue corresponding to position 265 in SEQ ID NO: 2; I, V, Q, C at a residue corresponding to position 266 in SEQ ID NO: 2; Y at a residue corresponding to position 267 in SEQ ID NO: 2; V, M, T, F, IMPR (SEQ ID NO: 42), IMNR (SEQ ID NO: 43) at a residue corresponding to position 268 in SEQ ID NO: 2; M, at a residue corresponding to position 269 in SEQ ID NO: 2; a deletion, P at a residue corresponding to position 270 in SEQ ID NO: 2; a deletion, R, Q at a residue corresponding to position 271 in SEQ ID NO: 2; W at a residue corresponding to position 272 in SEQ ID NO: 2; P, PEEMLRAIQ (SEQ ID NO: 44), PEEMLQAIQ (SEQ ID NO: 45) at a residue corresponding to position 274 in SEQ ID NO: 2; D, A, E, R, K, EEMLQAIQ (SEQ ID NO: 46), EEMLRAIQ (SEQ ID NO: 47), a deletion, KEMLQAIQ (SEQ ID NO: 48) at a residue corresponding to position 275 in SEQ ID NO: 2; a deletion at a residue corresponding to position 276 in SEQ ID NO: 2; a deletion, M, Y, L, F at a residue corresponding to position 277 in SEQ ID NO: 2; a deletion at a residue corresponding to position 278 in SEQ ID NO: 2; a deletion, Q, R, QAIQ (SEQ ID NO: 49), RAIQ (SEQ ID NO: 50) at a residue corresponding to position 279 in SEQ ID NO: 2; a deletion at a residue corresponding to position 280 in SEQ ID NO: 2; a deletion, V, M, L, I at a residue corresponding to position 281 in SEQ ID NO: 2; a deletion, Q at a residue corresponding to position 282 in SEQ ID NO: 2; P, C at a residue corresponding to position 286 in SEQ ID NO: 2; T, TVFFA (SEQ ID NO: 51), TSFFA (SEQ ID NO: 52), TFFFA (SEQ ID NO: 53), TMFFA (SEQ ID NO: 54), TVFHA (SEQ ID NO: 55) at a residue corresponding to position 287 in SEQ ID NO: 2; H, F, M, Y, R, I, V, T, C, a deletion at a residue corresponding to position 288 in SEQ ID NO: 2; a deletion, M, V, A, L, F, I at a residue corresponding to position 289 in SEQ ID NO: 2; a deletion, A, W, H, C, F, V, P, L at a residue corresponding to position 290 in SEQ ID NO: 2; a deletion, A, M, G, V at a residue corresponding to position 291 in SEQ ID NO: 2; A, T at a residue corresponding to position 294 in SEQ ID NO: 2; L, a deletion at a residue corresponding to position 295 in SEQ ID NO: 2; M, L at a residue corresponding to position 296 in SEQ ID NO: 2; M, Q, I, A, L, T at a residue corresponding to position 297 in SEQ ID NO: 2; M, A, F, R, IM at a residue corresponding to position 298 in SEQ ID NO: 2; M, F at a residue corresponding to position 299 in SEQ ID NO: 2; A at a residue corresponding to position 300 in SEQ ID NO: 2; M, R, E at a residue corresponding to position 304 in SEQ ID NO: 2; D at a residue corresponding to position 306 in SEQ ID NO: 2; L at a residue corresponding to position 309 in SEQ ID NO: 2; L, D at a residue corresponding to position 310 in SEQ ID NO: 2; L at a residue corresponding to position 311 in SEQ ID NO: 2; S, SHVRSV (SEQ ID NO: 56), SHVRTV (SEQ ID NO: 57) at a residue corresponding to position 314 in SEQ ID NO: 2; S, a deletion at a residue corresponding to position 315 in SEQ ID NO: 2; a deletion, V, L, I at a residue corresponding to position 316 in SEQ ID NO: 2; a deletion, H, N, Q, T at a residue corresponding to position 317 in SEQ ID NO: 2; a deletion, Y, M, T, S, C, Q, R, H, V, W, I, L at a residue corresponding to position 318 in SEQ ID NO: 2; a deletion, C, F, V, L, M at a residue corresponding to position 319 in SEQ ID NO: 2; F, I, V, Y, T, M, W at a residue corresponding to position 320 in SEQ ID NO: 2; C at a residue corresponding to position 321 in SEQ ID NO: 2; G at a residue corresponding to position 323 in SEQ ID NO: 2; S, M at a residue corresponding to position 324 in SEQ ID NO: 2; V, I, C at a residue corresponding to position 326 in SEQ ID NO: 2; S at a residue corresponding to position 327 in SEQ ID NO: 2; H, V, R, S, RELE (SEQ ID NO: 58) at a residue corresponding to position 328 in SEQ ID NO: 2; E, a deletion, ELE at a residue corresponding to position 329 in SEQ ID NO: 2; a deletion, L, T, I at a residue corresponding to position 330 in SEQ ID NO: 2; a deletion, M, E, C, L, I at a residue corresponding to position 331 in SEQ ID NO: 2; A, W, C, M, I at a residue corresponding to position 334 in SEQ ID NO: 2; Q, R at a residue corresponding to position 335 in SEQ ID NO: 2; K at a residue corresponding to position 336 in SEQ ID NO: 2; M at a residue corresponding to position 338 in SEQ ID NO: 2; S at a residue corresponding to position 339 in SEQ ID NO: 2; H, T at a residue corresponding to position 341 in SEQ ID NO: 2; L, F at a residue corresponding to position 342 in SEQ ID NO: 2; D, H, Y at a residue corresponding to position 348 in SEQ ID NO: 2; V, C, T, F, A, W at a residue corresponding to position 351 in SEQ ID NO: 2; E, K, D at a residue corresponding to position 352 in SEQ ID NO: 2; V, L, M at a residue corresponding to position 353 in SEQ ID NO: 2; C, F, L, Y, A, K, S, I at a residue corresponding to position 354 in SEQ ID NO: 2; E at a residue corresponding to position 355 in SEQ ID NO: 2; A at a residue corresponding to position 356 in SEQ ID NO: 2; W at a residue corresponding to position 357 in SEQ ID NO: 2; M at a residue corresponding to position 359 in SEQ ID NO: 2; S at a residue corresponding to position 360 in SEQ ID NO: 2; C, S at a residue corresponding to position 362 in SEQ ID NO: 2; A, T, G at a residue corresponding to position 363 in SEQ ID NO: 2; C, S at a residue corresponding to position 364 in SEQ ID NO: 2; L, I, M, C at a residue corresponding to position 365 in SEQ ID NO: 2; S, V, T at a residue corresponding to position 366 in SEQ ID NO: 2; A, C, S, H at a residue corresponding to position 367 in SEQ ID NO: 2; H at a residue corresponding to position 368 in SEQ ID NO: 2; H, Y, I, C, L, V, W, S, T, M at a residue corresponding to position 369 in SEQ ID NO: 2; Y, R, T, S, H, G, N, Q at a residue corresponding to position 370 in SEQ ID NO: 2; W at a residue corresponding to position 371 in SEQ ID NO: 2; K, W, Q, H, S, R, N at a residue corresponding to position 372 in SEQ ID NO: 2; S, H, P, Q, E at a residue corresponding to position 373 in SEQ ID NO: 2; D, T, A, R, S, P, W, E, Q, H at a residue corresponding to position 374 in SEQ ID NO: 2; P at a residue corresponding to position 375 in SEQ ID NO: 2; T, K, E, Q, H, D at a residue corresponding to position 376 in SEQ ID NO: 2; Y, R, N at a residue corresponding to position 377 in SEQ ID NO: 2; S, Y, P, V, A at a residue corresponding to position 378 in SEQ ID NO: 2; G at a residue corresponding to position 379 in SEQ ID NO: 2; T at a residue corresponding to position 380 in SEQ ID NO: 2; V, S, I at a residue corresponding to position 381 in SEQ ID NO: 2; Q, H, T, Y, W, P at a residue corresponding to position 383 in SEQ ID NO: 2; I, P, A, M at a residue corresponding to position 384 in SEQ ID NO: 2; S, A at a residue corresponding to position 386 in SEQ ID NO: 2; G, S, H at a residue corresponding to position 387 in SEQ ID NO: 2; T, M, V, L at a residue corresponding to position 388 in SEQ ID NO: 2; R, K, E at a residue corresponding to position 389 in SEQ ID NO: 2; C, L, A at a residue corresponding to position 390 in SEQ ID NO: 2; K at a residue corresponding to position 391 in SEQ ID NO: 2; D at a residue corresponding to position 394 in SEQ ID NO: 2; D, N at a residue corresponding to position 398 in SEQ ID NO: 2; V, L, I at a residue corresponding to position 402 in SEQ ID NO: 2; L, V at a residue corresponding to position 406 in SEQ ID NO: 2; D at a residue corresponding to position 407 in SEQ ID NO: 2; N, Q, NTENAPR (SEQ ID NO: 59), NTENAPD (SEQ ID NO: 60) at a residue corresponding to position 410 in SEQ ID NO: 2; T, TENAPD (SEQ ID NO: 61), TQNAPR (SEQ ID NO: 62), a deletion, TENAPR (SEQ ID NO: 63), TQNAPN (SEQ ID NO: 64) at a residue corresponding to position 411 in SEQ ID NO: 2; a deletion, Q at a residue corresponding to position 412 in SEQ ID NO: 2; a deletion, N, NNPN (SEQ ID NO: 65) at a residue corresponding to position 413 in SEQ ID NO: 2; a deletion, A, S, N at a residue corresponding to position 414 in SEQ ID NO: 2; a deletion, P at a residue corresponding to position 415 in SEQ ID NO: 2; a deletion, D, R, N, E at a residue corresponding to position 416 in SEQ ID NO: 2; P at a residue corresponding to position 418 in SEQ ID NO: 2; G at a residue corresponding to position 419 in SEQ ID NO: 2; E at a residue corresponding to position 420 in SEQ ID NO: 2; M, V at a residue corresponding to position 424 in SEQ ID NO: 2; V, L at a residue corresponding to position 425 in SEQ ID NO: 2; M, L at a residue corresponding to position 426 in SEQ ID NO: 2; T, N, S at a residue corresponding to position 430 in SEQ ID NO: 2; I, L at a residue corresponding to position 431 in SEQ ID NO: 2; T, F at a residue corresponding to position 432 in SEQ ID NO: 2; S, K at a residue corresponding to position 433 in SEQ ID NO: 2; W at a residue corresponding to position 436 in SEQ ID NO: 2; T at a residue corresponding to position 442 in SEQ ID NO: 2; R, K, RASFA (SEQ ID NO: 66) at a residue corresponding to position 443 in SEQ ID NO: 2; a deletion at a residue corresponding to position 444 in SEQ ID NO: 2; a deletion, M, T, S, A at a residue corresponding to position 445 in SEQ ID NO: 2; a deletion at a residue corresponding to position 446 in SEQ ID NO: 2; a deletion, A at a residue corresponding to position 447 in SEQ ID NO: 2; S, G at a residue corresponding to position 455 in SEQ ID NO: 2; G, E, D at a residue corresponding to position 459 in SEQ ID NO: 2; D, G at a residue corresponding to position 461 in SEQ ID NO: 2; R, G at a residue corresponding to position 462 in SEQ ID NO: 2; L, Y at a residue corresponding to position 464 in SEQ ID NO: 2; H at a residue corresponding to position 465 in SEQ ID NO: 2; M at a residue corresponding to position 469 in SEQ ID NO: 2; C, G at a residue corresponding to position 470 in SEQ ID NO: 2; Y, K, M, W, I, R at a residue corresponding to position 471 in SEQ ID NO: 2; I, F, C, Y, L, M at a residue corresponding to position 472 in SEQ ID NO: 2; E, EDGH (SEQ ID NO: 67), EDGY (SEQ ID NO: 68) at a residue corresponding to position 474 in SEQ ID NO: 2; D, a deletion at a residue corresponding to position 475 in SEQ ID NO: 2; a deletion at a residue corresponding to position 476 in SEQ ID NO: 2; a deletion, H, F, N, E, Y at a residue corresponding to position 477 in SEQ ID NO: 2; W, I, M at a residue corresponding to position 478 in SEQ ID NO: 2; T, H, Y, F, W at a residue corresponding to position 479 in SEQ ID NO: 2; V, L at a residue corresponding to position 480 in SEQ ID NO: 2; C, I, T at a residue corresponding to position 481 in SEQ ID NO: 2; L at a residue corresponding to position 484 in SEQ ID NO: 2; D at a residue corresponding to position 486 in SEQ ID NO: 2; I at a residue corresponding to position 489 in SEQ ID NO: 2; R, C, V at a residue corresponding to position 490 in SEQ ID NO: 2; G, R, N at a residue corresponding to position 491 in SEQ ID NO: 2; F, H, Y, L at a residue corresponding to position 493 in SEQ ID NO: 2; N at a residue corresponding to position 494 in SEQ ID NO: 2; F, S, Y at a residue corresponding to position 496 in SEQ ID NO: 2; L, V, I at a residue corresponding to position 503 in SEQ ID NO: 2; R, RHPD (SEQ ID NO: 69) at a residue corresponding to position 506 in SEQ ID NO: 2; a deletion at a residue corresponding to position 507 in SEQ ID NO: 2; a deletion, P at a residue corresponding to position 508 in SEQ ID NO: 2; a deletion, D at a residue corresponding to position 509 in SEQ ID NO: 2; A, V, C at a residue corresponding to position 513 in SEQ ID NO: 2; V at a residue corresponding to position 518 in SEQ ID NO: 2; P at a residue corresponding to position 519 in SEQ ID NO: 2; E, P at a residue corresponding to position 521 in SEQ ID NO: 2; Q, A, R, M, Y, D at a residue corresponding to position 523 in SEQ ID NO: 2; S, A at a residue corresponding to position 524 in SEQ ID NO: 2; S, T, D, G, E at a residue corresponding to position 525 in SEQ ID NO: 2; P, T, V at a residue corresponding to position 527 in SEQ ID NO: 2; A, V at a residue corresponding to position 528 in SEQ ID NO: 2; V, A at a residue corresponding to position 529 in SEQ ID NO: 2; L, V at a residue corresponding to position 539 in SEQ ID NO: 2; T at a residue corresponding to position 540 in SEQ ID NO: 2; E, D at a residue corresponding to position 542 in SEQ ID NO: 2; A, K at a residue corresponding to position 546 in SEQ ID NO: 2; K, R at a residue corresponding to position 551 in SEQ ID NO: 2; S at a residue corresponding to position 553 in SEQ ID NO: 2; S, P, H at a residue corresponding to position 554 in SEQ ID NO: 2; F at a residue corresponding to position 555 in SEQ ID NO: 2; K at a residue corresponding to position 557 in SEQ ID NO: 2; P at a residue corresponding to position 559 in SEQ ID NO: 2; G at a residue corresponding to position 560 in SEQ ID NO: 2; V, a deletion at a residue corresponding to position 562 in SEQ ID NO: 2; F at a residue corresponding to position 564 in SEQ ID NO: 2; V, F, FV at a residue corresponding to position 565 in SEQ ID NO: 2; E, VE at a residue corresponding to position 566 in SEQ ID NO: 2; Q, E at a residue corresponding to position 583 in SEQ ID NO: 2; R, E at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; K, D, P, N, R at a residue corresponding to position 10 in SEQ ID NO: 2; A, I, V, M at a residue corresponding to position 12 in SEQ ID NO: 2; C at a residue corresponding to position 13 in SEQ ID NO: 2; P, A, Q at a residue corresponding to position 14 in SEQ ID NO: 2; E, L, S, R at a residue corresponding to position 15 in SEQ ID NO: 2; C, L, I at a residue corresponding to position 16 in SEQ ID NO: 2; D, R at a residue corresponding to position 19 in SEQ ID NO: 2; H, K, R, D at a residue corresponding to position 20 in SEQ ID NO: 2; D, E at a residue corresponding to position 22 in SEQ ID NO: 2; N at a residue corresponding to position 25 in SEQ ID NO: 2; M at a residue corresponding to position 28 in SEQ ID NO: 2; C at a residue corresponding to position 37 in SEQ ID NO: 2; R at a residue corresponding to position 39 in SEQ ID NO: 2; T at a residue corresponding to position 40 in SEQ ID NO: 2; M, L, V, I at a residue corresponding to position 41 in SEQ ID NO: 2; S at a residue corresponding to position 43 in SEQ ID NO: 2; M at a residue corresponding to position 44 in SEQ ID NO: 2; I, V at a residue corresponding to position 45 in SEQ ID NO: 2; P at a residue corresponding to position 48 in SEQ ID NO: 2; I, F, R, V, L at a residue corresponding to position 50 in SEQ ID NO: 2; R at a residue corresponding to position 51 in SEQ ID NO: 2; L at a residue corresponding to position 53 in SEQ ID NO: 2; F at a residue corresponding to position 57 in SEQ ID NO: 2; V, L, M, F at a residue corresponding to position 58 in SEQ ID NO: 2; V at a residue corresponding to position 59 in SEQ ID NO: 2; P, E at a residue corresponding to position 61 in SEQ ID NO: 2; D at a residue corresponding to position 62 in SEQ ID NO: 2; D at a residue corresponding to position 63 in SEQ ID NO: 2; G at a residue corresponding to position 66 in SEQ ID NO: 2; K at a residue corresponding to position 67 in SEQ ID NO: 2; F, Y, L at a residue corresponding to position 69 in SEQ ID NO: 2; F, W at a residue corresponding to position 71 in SEQ ID NO: 2; A, R at a residue corresponding to position 72 in SEQ ID NO: 2; F at a residue corresponding to position 74 in SEQ ID NO: 2; D at a residue corresponding to position 75 in SEQ ID NO: 2; T, D, A, R at a residue corresponding to position 76 in SEQ ID NO: 2; R at a residue corresponding to position 77 in SEQ ID NO: 2; A at a residue corresponding to position 78 in SEQ ID NO: 2; K at a residue corresponding to position 80 in SEQ ID NO: 2; C at a residue corresponding to position 81 in SEQ ID NO: 2; S, H, Q, N at a residue corresponding to position 83 in SEQ ID NO: 2; Y, H at a residue corresponding to position 84 in SEQ ID NO: 2; F at a residue corresponding to position 85 in SEQ ID NO: 2; A, Q at a residue corresponding to position 86 in SEQ ID NO: 2; R at a residue corresponding to position 87 in SEQ ID NO: 2; L at a residue corresponding to position 88 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; A at a residue corresponding to position 97 in SEQ ID NO: 2; M at a residue corresponding to position 98 in SEQ ID NO: 2; L at a residue corresponding to position 99 in SEQ ID NO: 2; C at a residue corresponding to position 100 in SEQ ID NO: 2; P at a residue corresponding to position 101 in SEQ ID NO: 2; R at a residue corresponding to position 103 in SEQ ID NO: 2; L, W, A at a residue corresponding to position 104 in SEQ ID NO: 2; W, F at a residue corresponding to position 106 in SEQ ID NO: 2; V, P, I, L, W, F at a residue corresponding to position 107 in SEQ ID NO: 2; V at a residue corresponding to position 108 in SEQ ID NO: 2; V at a residue corresponding to position 109 in SEQ ID NO: 2; M, F, Y, A, D at a residue corresponding to position 110 in SEQ ID NO: 2; T, C, V at a residue corresponding to position 113 in SEQ ID NO: 2; M, W, L, G, A at a residue corresponding to position 114 in SEQ ID NO: 2; A at a residue corresponding to position 115 in SEQ ID NO: 2; I at a residue corresponding to position 116 in SEQ ID NO: 2; V, T, I at a residue corresponding to position 119 in SEQ ID NO: 2; M, F, V, A, L, W, Y at a residue corresponding to position 120 in SEQ ID NO: 2; T at a residue corresponding to position 121 in SEQ ID NO: 2; A at a residue corresponding to position 122 in SEQ ID NO: 2; M, V, I at a residue corresponding to position 123 in SEQ ID NO: 2; Y, S, N at a residue corresponding to position 124 in SEQ ID NO: 2; P, T, W at a residue corresponding to position 125 in SEQ ID NO: 2; M, N, R, A at a residue corresponding to position 126 in SEQ ID NO: 2; F, W, L at a residue corresponding to position 127 in SEQ ID NO: 2; R at a residue corresponding to position 128 in SEQ ID NO: 2; M at a residue corresponding to position 130 in SEQ ID NO: 2; E at a residue corresponding to position 131 in SEQ ID NO: 2; E, Q, K at a residue corresponding to position 136 in SEQ ID NO: 2; M at a residue corresponding to position 137 in SEQ ID NO: 2; M, C, I at a residue corresponding to position 140 in SEQ ID NO: 2; M at a residue corresponding to position 141 in SEQ ID NO: 2; N, Q at a residue corresponding to position 142 in SEQ ID NO: 2; G, R, D at a residue corresponding to position 145 in SEQ ID NO: 2; S at a residue corresponding to position 147 in SEQ ID NO: 2; L, V, A at a residue corresponding to position 148 in SEQ ID NO: 2; V, L at a residue corresponding to position 149 in SEQ ID NO: 2; V at a residue corresponding to position 150 in SEQ ID NO: 2; C at a residue corresponding to position 151 in SEQ ID NO: 2; L at a residue corresponding to position 157 in SEQ ID NO: 2; R, E at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 165 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, V, L at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 189 in SEQ ID NO: 2; V at a residue corresponding to position 190 in SEQ ID NO: 2; A, E at a residue corresponding to position 191 in SEQ ID NO: 2; T at a residue corresponding to position 192 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; P at a residue corresponding to position 196 in SEQ ID NO: 2; A, P, L at a residue corresponding to position 201 in SEQ ID NO: 2; T at a residue corresponding to position 205 in SEQ ID NO: 2; D at a residue corresponding to position 208 in SEQ ID NO: 2; I, T, P, V at a residue corresponding to position 209 in SEQ ID NO: 2; L, T, C, M, V, A at a residue corresponding to position 211 in SEQ ID NO: 2; L, M at a residue corresponding to position 212 in SEQ ID NO: 2; I, C, V, Q, M at a residue corresponding to position 213 in SEQ ID NO: 2; Y at a residue corresponding to position 214 in SEQ ID NO: 2; S at a residue corresponding to position 215 in SEQ ID NO: 2; G at a residue corresponding to position 216 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; T, M, L, R, K, H at a residue corresponding to position 221 in SEQ ID NO: 2; S at a residue corresponding to position 222 in SEQ ID NO: 2; A at a residue corresponding to position 224 in SEQ ID NO: 2; A at a residue corresponding to position 225 in SEQ ID NO: 2; M, I, V at a residue corresponding to position 226 in SEQ ID NO: 2; L, D, H, I at a residue corresponding to position 227 in SEQ ID NO: 2; S at a residue corresponding to position 228 in SEQ ID NO: 2; H, K at a residue corresponding to position 230 in SEQ ID NO: 2; N at a residue corresponding to position 231 in SEQ ID NO: 2; L, I, V at a residue corresponding to position 232 in SEQ ID NO: 2; L, V, M, W, A, I at a residue corresponding to position 233 in SEQ ID NO: 2; S, W at a residue corresponding to position 234 in SEQ ID NO: 2; Q, S at a residue corresponding to position 235 in SEQ ID NO: 2; A, C, I, M, V at a residue corresponding to position 236 in SEQ ID NO: 2; A, M, Q, R, E at a residue corresponding to position 237 in SEQ ID NO: 2; M, S, W, N, H at a residue corresponding to position 238 in SEQ ID NO: 2; F, M, V, C, L at a residue corresponding to position 239 in SEQ ID NO: 2; A, R at a residue corresponding to position 240 in SEQ ID NO: 2; A, H, E, Q at a residue corresponding to position 241 in SEQ ID NO: 2; V, C, T, W at a residue corresponding to position 242 in SEQ ID NO: 2; I, F, M, W, V at a residue corresponding to position 243 in SEQ ID NO: 2; E, P, Q, H, G at a residue corresponding to position 244 in SEQ ID NO: 2; M, L, H, W, Y at a residue corresponding to position 245 in SEQ ID NO: 2; I, T, M at a residue corresponding to position 246 in SEQ ID NO: 2; R, P, E at a residue corresponding to position 247 in SEQ ID NO: 2; E at a residue corresponding to position 251 in SEQ ID NO: 2; H, V at a residue corresponding to position 252 in SEQ ID NO: 2; Y, M, F, L, V at a residue corresponding to position 253 in SEQ ID NO: 2; I, F, V, M at a residue corresponding to position 254 in SEQ ID NO: 2; A, T, N, L, I, G, H, C, V, S at a residue corresponding to position 255 in SEQ ID NO: 2; F, V, I, C, Y at a residue corresponding to position 256 in SEQ ID NO: 2; M at a residue corresponding to position 257 in SEQ ID NO: 2; L, F at a residue corresponding to position 259 in SEQ ID NO: 2; H, S, Y, F at a residue corresponding to position 260 in SEQ ID NO: 2; C, T, A, I, M at a residue corresponding to position 262 in SEQ ID NO: 2; A, Y, H at a residue corresponding to position 263 in SEQ ID NO: 2; A at a residue corresponding to position 264 in SEQ ID NO: 2; M, L, W, F, C at a residue corresponding to position 265 in SEQ ID NO: 2; N, T, M, V at a residue corresponding to position 266 in SEQ ID NO: 2; I, G, M, W, F, L, V, C, A at a residue corresponding to position 267 in SEQ ID NO: 2; M, A, C, G, V at a residue corresponding to position 268 in SEQ ID NO: 2; L, M, I, V at a residue corresponding to position 269 in SEQ ID NO: 2; M, H, Y, T at a residue corresponding to position 270 in SEQ ID NO: 2; A, P, C, T, L at a residue corresponding to position 271 in SEQ ID NO: 2; C, V, P, A, T at a residue corresponding to position 272 in SEQ ID NO: 2; M, V, F, I at a residue corresponding to position 273 in SEQ ID NO: 2; Y, R, V, M, C, H, W at a residue corresponding to position 274 in SEQ ID NO: 2; G, C, S, H at a residue corresponding to position 275 in SEQ ID NO: 2; A, C at a residue corresponding to position 277 in SEQ ID NO: 2; C, T, A, H at a residue corresponding to position 278 in SEQ ID NO: 2; V, M, I at a residue corresponding to position 279 in SEQ ID NO: 2; F, L, V, M at a residue corresponding to position 281 in SEQ ID NO: 2; P, M, E, A, H at a residue corresponding to position 282 in SEQ ID NO: 2; E, D, P, R at a residue corresponding to position 283 in SEQ ID NO: 2; F, R, W at a residue corresponding to position 284 in SEQ ID NO: 2; R, F, W, D at a residue corresponding to position 285 in SEQ ID NO: 2; D, F at a residue corresponding to position 286 in SEQ ID NO: 2; P, A, V at a residue corresponding to position 287 in SEQ ID NO: 2; A at a residue corresponding to position 290 in SEQ ID NO: 2; M, V, L, P, I at a residue corresponding to position 291 in SEQ ID NO: 2; H at a residue corresponding to position 292 in SEQ ID NO: 2; I, L, A at a residue corresponding to position 294 in SEQ ID NO: 2; W, M, V, I at a residue corresponding to position 295 in SEQ ID NO: 2; D, Q at a residue corresponding to position 296 in SEQ ID NO: 2; M, V, L at a residue corresponding to position 298 in SEQ ID NO: 2; A at a residue corresponding to position 299 in SEQ ID NO: 2; Q at a residue corresponding to position 300 in SEQ ID NO: 2; Q at a residue corresponding to position 302 in SEQ ID NO: 2; V, I, A, P, C at a residue corresponding to position 303 in SEQ ID NO: 2; S at a residue corresponding to position 304 in SEQ ID NO: 2; W, Y, V, I, F, H, M at a residue corresponding to position 305 in SEQ ID NO: 2; V, Y, I, M, L, F at a residue corresponding to position 306 in SEQ ID NO: 2; P, W, Y, H, M at a residue corresponding to position 307 in SEQ ID NO: 2; T, A, S, L, G, V, M at a residue corresponding to position 308 in SEQ ID NO: 2; A, R, P, T at a residue corresponding to position 311 in SEQ ID NO: 2; I, M, V, F at a residue corresponding to position 312 in SEQ ID NO: 2; L, F, W at a residue corresponding to position 313 in SEQ ID NO: 2; Q, E, H at a residue corresponding to position 314 in SEQ ID NO: 2; R, A, K, L at a residue corresponding to position 315 in SEQ ID NO: 2; W, M, I, F at a residue corresponding to position 316 in SEQ ID NO: 2; H, D at a residue corresponding to position 318 in SEQ ID NO: 2; R, Q at a residue corresponding to position 321 in SEQ ID NO: 2; Q, K, A, H at a residue corresponding to position 322 in SEQ ID NO: 2; L, F at a residue corresponding to position 330 in SEQ ID NO: 2; G at a residue corresponding to position 331 in SEQ ID NO: 2; S at a residue corresponding to position 333 in SEQ ID NO: 2; I, M, V at a residue corresponding to position 334 in SEQ ID NO: 2; R, K at a residue corresponding to position 335 in SEQ ID NO: 2; T, I, M, Y, V, F, W at a residue corresponding to position 336 in SEQ ID NO: 2; L, M, F, A, V at a residue corresponding to position 337 in SEQ ID NO: 2; W, L, M, V at a residue corresponding to position 338 in SEQ ID NO: 2; F, T, S, C at a residue corresponding to position 339 in SEQ ID NO: 2; G at a residue corresponding to position 340 in SEQ ID NO: 2; A, S at a residue corresponding to position 341 in SEQ ID NO: 2; S at a residue corresponding to position 342 in SEQ ID NO: 2; P, A at a residue corresponding to position 343 in SEQ ID NO: 2; M, I, V, C at a residue corresponding to position 344 in SEQ ID NO: 2; S at a residue corresponding to position 345 in SEQ ID NO: 2; V, A, P, E at a residue corresponding to position 346 in SEQ ID NO: 2; E at a residue corresponding to position 347 in SEQ ID NO: 2; T, I, L at a residue corresponding to position 348 in SEQ ID NO: 2; M, F, L, A, I, Y at a residue corresponding to position 349 in SEQ ID NO: 2; F, R, Q, W at a residue corresponding to position 351 in SEQ ID NO: 2; I, M, Y at a residue corresponding to position 352 in SEQ ID NO: 2; R at a residue corresponding to position 353 in SEQ ID NO: 2; K at a residue corresponding to position 354 in SEQ ID NO: 2; A, R at a residue corresponding to position 355 in SEQ ID NO: 2; W, G, V, L, P, I, F, A at a residue corresponding to position 357 in SEQ ID NO: 2; Q at a residue corresponding to position 358 in SEQ ID NO: 2; H, Q at a residue corresponding to position 359 in SEQ ID NO: 2; F at a residue corresponding to position 361 in SEQ ID NO: 2; H, L, C at a residue corresponding to position 362 in SEQ ID NO: 2; D, V, I, G at a residue corresponding to position 363 in SEQ ID NO: 2; M, V, I at a residue corresponding to position 365 in SEQ ID NO: 2; I, V, L, F at a residue corresponding to position 366 in SEQ ID NO: 2; T, C, M, I, V, L at a residue corresponding to position 367 in SEQ ID NO: 2; N, Q, E, T at a residue corresponding to position 368 in SEQ ID NO: 2; G, A at a residue corresponding to position 369 in SEQ ID NO: 2; Y, F at a residue corresponding to position 370 in SEQ ID NO: 2; L, Q at a residue corresponding to position 372 in SEQ ID NO: 2; S at a residue corresponding to position 373 in SEQ ID NO: 2; A, C at a residue corresponding to position 375 in SEQ ID NO: 2; T, S, C, G at a residue corresponding to position 376 in SEQ ID NO: 2; G, M at a residue corresponding to position 377 in SEQ ID NO: 2; C, T, V at a residue corresponding to position 378 in SEQ ID NO: 2; C, V, I, L, A at a residue corresponding to position 379 in SEQ ID NO: 2; F, C, H, S, M, T, V at a residue corresponding to position 380 in SEQ ID NO: 2; N, F, L, C, V at a residue corresponding to position 381 in SEQ ID NO: 2; L, N, C, T, P at a residue corresponding to position 382 in SEQ ID NO: 2; D, N, R, P, Y at a residue corresponding to position 383 in SEQ ID NO: 2; P, N, D, W at a residue corresponding to position 384 in SEQ ID NO: 2; G, D at a residue corresponding to position 385 in SEQ ID NO: 2; D, V, L, T at a residue corresponding to position 386 in SEQ ID NO: 2; D, R, N, H, E at a residue corresponding to position 387 in SEQ ID NO: 2; K, H at a residue corresponding to position 388 in SEQ ID NO: 2; A, P, L at a residue corresponding to position 389 in SEQ ID NO: 2; S, T at a residue corresponding to position 391 in SEQ ID NO: 2; V, C at a residue corresponding to position 392 in SEQ ID NO: 2; R, K, I, Q, L at a residue corresponding to position 394 in SEQ ID NO: 2; A at a residue corresponding to position 395 in SEQ ID NO: 2; F, I, M, V at a residue corresponding to position 396 in SEQ ID NO: 2; D, H, N at a residue corresponding to position 398 in SEQ ID NO: 2; M, V, T, C, I at a residue corresponding to position 399 in SEQ ID NO: 2; D, Q at a residue corresponding to position 400 in SEQ ID NO: 2; M, I, C, L, A at a residue corresponding to position 401 in SEQ ID NO: 2; R at a residue corresponding to position 402 in SEQ ID NO: 2; V at a residue corresponding to position 404 in SEQ ID NO: 2; D at a residue corresponding to position 405 in SEQ ID NO: 2; E at a residue corresponding to position 406 in SEQ ID NO: 2; P at a residue corresponding to position 407 in SEQ ID NO: 2; Q, E, G at a residue corresponding to position 409 in SEQ ID NO: 2; D, S, V, G, N at a residue corresponding to position 411 in SEQ ID NO: 2; I, V at a residue corresponding to position 413 in SEQ ID NO: 2; W, L, V, C, Q at a residue corresponding to position 414 in SEQ ID NO: 2; I at a residue corresponding to position 415 in SEQ ID NO: 2; R at a residue corresponding to position 416 in SEQ ID NO: 2; S at a residue corresponding to position 417 in SEQ ID NO: 2; D at a residue corresponding to position 418 in SEQ ID NO: 2; H, S, Q, M at a residue corresponding to position 419 in SEQ ID NO: 2; V at a residue corresponding to position 420 in SEQ ID NO: 2; T, F at a residue corresponding to position 421 in SEQ ID NO: 2; H, L at a residue corresponding to position 425 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; T, V at a residue corresponding to position 434 in SEQ ID NO: 2; I, M, L at a residue corresponding to position 435 in SEQ ID NO: 2; F, M, Y at a residue corresponding to position 441 in SEQ ID NO: 2; K, H, Y, R, C at a residue corresponding to position 442 in SEQ ID NO: 2; S at a residue corresponding to position 443 in SEQ ID NO: 2; M, I, L, V at a residue corresponding to position 446 in SEQ ID NO: 2; A, G at a residue corresponding to position 447 in SEQ ID NO: 2; I at a residue corresponding to position 449 in SEQ ID NO: 2; D, M at a residue corresponding to position 450 in SEQ ID NO: 2; E at a residue corresponding to position 451 in SEQ ID NO: 2; P at a residue corresponding to position 452 in SEQ ID NO: 2; G at a residue corresponding to position 453 in SEQ ID NO: 2; E, G, R at a residue corresponding to position 455 in SEQ ID NO: 2; D, E, Q, N at a residue corresponding to position 456 in SEQ ID NO: 2; N, F, Y, W at a residue corresponding to position 458 in SEQ ID NO: 2; Y at a residue corresponding to position 459 in SEQ ID NO: 2; Y, F, I, R, W at a residue corresponding to position 460 in SEQ ID NO: 2; I, L, Y at a residue corresponding to position 461 in SEQ ID NO: 2; I, T, V at a residue corresponding to position 462 in SEQ ID NO: 2; D at a residue corresponding to position 463 in SEQ ID NO: 2; D at a residue corresponding to position 464 in SEQ ID NO: 2; L at a residue corresponding to position 465 in SEQ ID NO: 2; D, K, A at a residue corresponding to position 466 in SEQ ID NO: 2; K at a residue corresponding to position 467 in SEQ ID NO: 2; M at a residue corresponding to position 468 in SEQ ID NO: 2; L at a residue corresponding to position 469 in SEQ ID NO: 2; V at a residue corresponding to position 470 in SEQ ID NO: 2; T, V at a residue corresponding to position 471 in SEQ ID NO: 2; S, N at a residue corresponding to position 473 in SEQ ID NO: 2; E at a residue corresponding to position 474 in SEQ ID NO: 2; N, Y, L, F, V, I at a residue corresponding to position 476 in SEQ ID NO: 2; T, L at a residue corresponding to position 478 in SEQ ID NO: 2; P, V at a residue corresponding to position 479 in SEQ ID NO: 2; L, I, S, P, A, V at a residue corresponding to position 480 in SEQ ID NO: 2; P, E at a residue corresponding to position 481 in SEQ ID NO: 2; E at a residue corresponding to position 483 in SEQ ID NO: 2; A, L, E, R at a residue corresponding to position 485 in SEQ ID NO: 2; C at a residue corresponding to position 490 in SEQ ID NO: 2; P at a residue corresponding to position 492 in SEQ ID NO: 2; F at a residue corresponding to position 493 in SEQ ID NO: 2; H at a residue corresponding to position 496 in SEQ ID NO: 2; A at a residue corresponding to position 497 in SEQ ID NO: 2; C at a residue corresponding to position 498 in SEQ ID NO: 2; P at a residue corresponding to position 502 in SEQ ID NO: 2; H at a residue corresponding to position 504 in SEQ ID NO: 2; Y, V, W at a residue corresponding to position 507 in SEQ ID NO: 2; V at a residue corresponding to position 508 in SEQ ID NO: 2; A at a residue corresponding to position 510 in SEQ ID NO: 2; V, L at a residue corresponding to position 511 in SEQ ID NO: 2; I at a residue corresponding to position 512 in SEQ ID NO: 2; V at a residue corresponding to position 513 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; L, M at a residue corresponding to position 526 in SEQ ID NO: 2; L at a residue corresponding to position 527 in SEQ ID NO: 2; R, H at a residue corresponding to position 532 in SEQ ID NO: 2; L at a residue corresponding to position 535 in SEQ ID NO: 2; Q at a residue corresponding to position 540 in SEQ ID NO: 2; Y, W at a residue corresponding to position 542 in SEQ ID NO: 2; N at a residue corresponding to position 546 in SEQ ID NO: 2; Q at a residue corresponding to position 552 in SEQ ID NO: 2; E at a residue corresponding to position 554 in SEQ ID NO: 2; F at a residue corresponding to position 558 in SEQ ID NO: 2; D at a residue corresponding to position 560 in SEQ ID NO: 2; D at a residue corresponding to position 561 in SEQ ID NO: 2; F, L, W at a residue corresponding to position 563 in SEQ ID NO: 2; D, K, S, L, T, R at a residue corresponding to position 564 in SEQ ID NO: 2; M, I, V, P, T at a residue corresponding to position 565 in SEQ ID NO: 2; A, E, D, P at a residue corresponding to position 566 in SEQ ID NO: 2; K, Q, T, N, S, A at a residue corresponding to position 567 in SEQ ID NO: 2; G, N, D at a residue corresponding to position 568 in SEQ ID NO: 2; M, K at a residue corresponding to position 569 in SEQ ID NO: 2; V, L at a residue corresponding to position 570 in SEQ ID NO: 2; R, P at a residue corresponding to position 572 in SEQ ID NO: 2; T, S at a residue corresponding to position 573 in SEQ ID NO: 2; M, L at a residue corresponding to position 574 in SEQ ID NO: 2; A, Y, K, T at a residue corresponding to position 575 in SEQ ID NO: 2; Y, A, L at a residue corresponding to position 577 in SEQ ID NO: 2; Q, A at a residue corresponding to position 578 in SEQ ID NO: 2; D at a residue corresponding to position 583 in SEQ ID NO: 2; N at a residue corresponding to position 584 in SEQ ID NO: 2; H, L at a residue corresponding to position 585 in SEQ ID NO: 2; A at a residue corresponding to position 587 in SEQ ID NO: 2; Q at a residue corresponding to position 588 in SEQ ID NO: 2; Q, E at a residue corresponding to position 591 in SEQ ID NO: 2; A at a residue corresponding to position 593 in SEQ ID NO: 2; E at a residue corresponding to position 595 in SEQ ID NO: 2; K at a residue corresponding to position 596 in SEQ ID NO: 2; D at a residue corresponding to position 598 in SEQ ID NO: 2; S at a residue corresponding to position 599 in SEQ ID NO: 2; L at a residue corresponding to position 601 in SEQ ID NO: 2; or any combination thereof. It is understood that when listed herein, alterations listed for a certain position can be in the alternative or in the conjunctive depending on the context. For example, the V, F, FV alterations at a residue corresponding to position 565 in SEQ ID NO: 2, indicates that alterations at a residue corresponding to position 565 in SEQ ID NO: 2 can comprise V or F or FV, wherein FV is F and V.

In some embodiments, an engineered ACL described herein has one or more amino acid alterations comprising: I4M, N7D, N7K, N7Q, N7S, N7T, A8I, A8S, T9A, T9K, T9S, I10F, I10H, I10P, I10V, I10W, I10Y, N12D, N12E, N12H, N12P, I13C, I13M, V15D, V15I, V15L, V15P, D16E, D16H, D16N, Y19F, Y19L, Y19P, Y19Q, D20W, K22S, G30D, G31A, E35G, E35P, T36H, S39A, V40C, V40F, V40L, V40M, A43G, K44D, K44H, K44S, K44T, Q47R, Q47RTYTQKEL (“RTYTQKEL” disclosed as SEQ ID NO: 15), Q47RTYTQREL (“RTYTQREL” disclosed as SEQ ID NO: 16), Q47RTYT (“RTYT” disclosed as SEQ ID NO: 17), d48, d49, L49Y, d50, S50T, d51, Q51F, Q51H, Q51L, Q51R, Q51W, Q51Y, d52, A52K, A52R, d53, d54, M54L, W55L, K60R, Y61F, Y61L, L64A, L64G, S66R, S67A, Y69L, C72G, C72T, R73F, R73G, R73L, R73V, H74K, D76T, E78V, D80G, P81PG, S82A, S82E, S82G, S82N, S82R, S82T, d83, M83K, M83P, M83R, M83S, V86I, L87I, L87V, L87VAV, d88, I88A, d89, T89I, T89L, T89V, F90C, F90L, F90M, F90V, F90W, F91A, F91L, F91M, F91S, V94C, V94CIEAA (“CIEAA” disclosed as SEQ ID NO: 18), V94S, V94T, V94TIEAA (“TIEAA” disclosed as SEQ ID NO: 19), d95, d96, L96E, d97, d98, P98A, P98L, V99C, V99M, V100S, V100M, V100L, H101F, H101M, W102H, W102F, W102L, A103G, A104V, A104L, A104T, L105V, L105W, L105H, L105A, L105I, D106S, D106Y, D106R, D106K, L107A, L107C, L107V, A109L, A109V, A109Q, I111V, I111A, I111M, I111L, I111C, I111VTPAN (“VTPAN” disclosed as SEQ ID NO: 20), S112T, d112, d113, P113L, d114, G114A, G114L, G114V, d115, S115N, T116A, T116W, T116P, G117S, S119T, Q121R, Q121D, Q126H, F127M, F127L, F127MRHTG (“MRHTG” disclosed as SEQ ID NO: 21), F127MRITG (“MRITG” disclosed as SEQ ID NO: 22), d128, d129, V129I, V129H, d130, d131, T131G, T131D, V136L, Y137T, A138D, A138S, K139E, A140E, A140K, A140D, V144T, T149R, K150I, K150L, Y152A, Y152T, Y152C, Y152I, N153G, N153S, A159G, E162S, E162H, E162R, E162SIDAF (“SIDAF” disclosed as SEQ ID NO: 23), E162RIESF (“RIESF” disclosed as SEQ ID NO: 24), E162HFDSF (“HFDSF” disclosed as SEQ ID NO: 25), E162HFDAF (“HFDAF” disclosed as SEQ ID NO: 26), E162HIDSF (“HIDSF” disclosed as SEQ ID NO: 27), L163F, L163I, d163, d164, E164D, d165, A165S, d166, L166F, D167L, A170G, M172L, M172A, M172D, R182Q, H189E, H189G, S191A, R194D, I195T, I195P, I195M, A196T, A196I, Y197W, Y197C, L198Y, L198V, L198S, L198W, L198I, L198M, L198T, G199M, G199L, G199W, G199P, G199F, G199C, G199A, M200W, S201F, S201T, S201N, S201P, S201Y, S201L, S202T, S202H, S202A, S202Q, S202E, S202L, S202G, S205T, V208F, S209P, A211G, V212A, V212C, R213M, R213V, R213E, L214V, T215S, S217Q, S217R, S217Y, S217H, S217K, S217G, T220W, T220M, T220V, S221T, S221A, C222S, C222N, S223A, S223N, S223I, S223L, S223H, E224T, E224Q, E224H, E224I, E224N, E224M, I225L, I225A, I225F, I225Q, S226Y, S226C, S226T, S226W, N227G, N227E, N227M, N227T, N227A, N227GKWSQD (“GKWSQD” disclosed as SEQ ID NO: 28), N227GKWSQSY (“GKWSQSY” disclosed as SEQ ID NO: 29), N227GKWSQ (“GKWSQ” disclosed as SEQ ID NO: 30), N227GKWSQDLK (“GKWSQDLK” disclosed as SEQ ID NO: 31), N227GKWSQQLQ (“GKWSQQLQ” disclosed as SEQ ID NO: 32), d228, d229, A229F, A229S, A229L, A229G, A229W, A229I, A229M, A229H, d230, S230R, S230M, S230P, S230T, S230E, S230Q, S230A, d231, E231Q, E231P, E231H, E231QDLQ (“QDLQ” disclosed as SEQ ID NO: 33), d232, S232W, S232A, S232Y, S232N, S232K, S232Q, S232D, d233, L233W, L233R, L233M, L233Y, L233P, d234, A234F, A234Y, A234T, A234K, A234R, A234Q, A234P, A234G, A234H, A234E, T235H, T235S, T235R, T235K, T235M, T235Q, T235D, T235G, Q238P, Q238N, Q238H, Q238T, Q238D, Q238DISAC (“DISAC” disclosed as SEQ ID NO: 34), Q238DVS, I239V, I239F, I239R, d239, d240, A240F, A240H, A240T, A240W, A240S, A240L, A240C, A240V, d241, A241C, A241M, A241P, A241I, d242, A242G, A242V, A242C, I243L, I243C, I243M, I244M, I244V, V246W, V246F, V246L, V246Y, S247F, S247C, H248S, H248P, L249A, L249V, L249M, F250M, F250A, F250W, G251A, G251H, L252I, L252M, L252W, L252Q, S253T, S253Q, S253N, S253I, S253H, S253V, S253M, K254E, K254I, K254T, K254Y, K254H, K254V, K254L, K254P, K254A, K254M, K254F, K254S, K254G, F255M, F255T, F255I, F255N, F255Y, F255C, F255TLLANV (“ILLANV” disclosed as SEQ ID NO: 35), F255TLLANVHY (“ILLANVHY” disclosed as SEQ ID NO: 36), F255TLLLNVHY (“ILLLNVHY” disclosed as SEQ ID NO: 37), F255TLLLNV (“ILLLNV” disclosed as SEQ ID NO: 38), F255ILLLNVHK (“ILLLNVHK” disclosed as SEQ ID NO: 39), F255ILLHNVHK (“ILLHNVHK” disclosed as SEQ ID NO: 40), F255ILLSNVHY (“ILLSNVHY” disclosed as SEQ ID NO: 41), L256I, L256V, L256H, L256C, L256F, d256, d257, I257L, I257T, I257N, d258, G258S, G258L, G258P, G258M, G258H, G258T, G258A, d259, N259C, N259A, d260, P260F, P260L, P260M, P260V, d261, H261Y, H261M, H261W, H261A, H261F, H261L, H261S, d262, A262M, A262K, A262Y, A262R, A262G, A262C, A262L, A262V, A262S, A262N, A264G, A264W, A264I, A264S, A264V, T265R, T265C, T265H, T265K, T266I, T266V, T266Q, T266C, V267Y, Y268V, Y268M, Y268T, Y268F, Y268IMPR (“IMPR” disclosed as SEQ ID NO: 42), Y268IMNR (“IMNR” disclosed as SEQ ID NO: 43), H269M, d269, d270, N270P, d271, G271R, G271Q, F272W, L274P, L274PEEMLRAIQ (“PEEMLRAIQ” disclosed as SEQ ID NO: 44), L274PEEMLQAIQ (“PEEMLQAIQ” disclosed as SEQ ID NO: 45), 1275D, I275A, 1275E, I275R, I275K, I275EEMLQAIQ (“EEMLQAIQ” disclosed as SEQ ID NO: 46), I275EEMLRAIQ (“EEMLRAIQ” disclosed as SEQ ID NO: 47), d275-282, I275KEMLQAIQ (“KEMLQAIQ” disclosed as SEQ ID NO: 48), d276, d277, V277M, V277Y, V277L, V277F, d278, d279, E279Q, E279R, E279QAIQ (“QAIQ” disclosed as SEQ ID NO: 49), E279RAIQ (“RAIQ” disclosed as SEQ ID NO: 50), d280, d281, Q281V, Q281M, Q281L, Q281I, d282, K282Q, V286P, V286C, N287T, N287TVFFA (“TVFFA” disclosed as SEQ ID NO: 51), N287TSFFA (“TSFFA” disclosed as SEQ ID NO: 52), N287TFFFA (“TFFFA” disclosed as SEQ ID NO: 53), N287TMFFA (“TMFFA” disclosed as SEQ ID NO: 54), N287TVFHA (“TVFHA” disclosed as SEQ ID NO: 55), S288H, S288F, S288M, S288Y, S288R, S288I, S288V, S288T, S288C, d288, d289, W289M, W289V, W289A, W289L, W289F, W289I, d290, T290A, T290W, T290H, T290C, T290F, T290V, T290P, T290L, d291, L291A, L291M, L291G, L291V, P294A, P294T, I295L, d295, I296M, I296L, V297M, V297Q, V297I, V297A, V297L, V297T, L298M, L298A, L298F, L298R, L298IM, L299M, L299F, T300A, I304M, I304R, 1304E, E306D, I309L, P310L, P310D, S311L, A314S, A314SHVRSV (“SHVRSV” disclosed as SEQ ID NO: 56), A314SHVRTV (“SHVRTV” disclosed as SEQ ID NO: 57), H315S, d315, d316, M316V, M316L, M316I, d317, R317H, R317N, R317Q, R317T, d318, A318Y, A318M, A318T, A318S, A318C, A318Q, A318R, A318H, A318V, A318W, A318I, A318L, d319, I319C, I319F, I319V, I319L, I319M, L320F, L320I, L320V, L320Y, L320T, L320M, L320W, S321C, A323G, A324S, A324M, L326V, L326I, L326C, G327S, A328H, A328V, A328R, A328S, A328RELE (“RELE” disclosed as SEQ ID NO: 58), N329E, d329, N329ELE, d330, V330L, V330T, V330I, d331, T331M, T331E, T331C, T331L, T331I, L334A, L334W, L334C, L334M, L334I, L335Q, L335R, T336K, V338M, T339S, N341H, N341T, K342L, K342F, E348D, E348H, E348Y, L351V, L351C, L351T, L351F, L351A, L351W, R352E, R352K, R352D, I353V, I353L, I353M, V354C, V354F, V354L, V354Y, V354A, V354K, V354S, V354I, Q355E, G356A, Y357W, L359M, T360S, T362C, T362S, S363A, S363T, S363G, P364C, P364S, V365L, V365I, V365M, V365C, A366S, A366V, A366T, T367A, T367C, T367S, T367H, L368H, F369H, F369Y, F369I, F369C, F369L, F369V, F369W, F369S, F369T, F369M, D370Y, D370R, D370T, D370S, D370H, D370G, D370N, D370Q, P371W, E372K, E372W, E372Q, E372H, E372S, E372R, E372N, D373S, D373H, D373P, D373Q, D373E, K374D, K374T, K374A, K374R, K374S, K374P, K374W, K374E, K374Q, K374H, E375P, R376T, R376K, R376E, R376Q, R376H, R376D, H377Y, H377R, H377N, I378S, 1378Y, 1378P, 1378V, 1378A, R379G, S380T, C381V, C381S, C381I, K383Q, K383H, K383T, K383Y, K383W, K383P, L384I, L384P, L384A, L384M, P386S, P386A, N387G, N387S, N387H, S388T, S388M, S388V, S388L, Q389R, Q389K, Q389E, V390C, V390L, V390A, R391K, N394D, V398D, V398N, A402V, A402L, A402I, D406L, D406V, P407D, L410N, L410Q, L410NTENAPR (“NTENAPR” disclosed as SEQ ID NO: 59), L410NTENAPD (“NTENAPD” disclosed as SEQ ID NO: 60), D411T, D411TENAPD (“TENAPD” disclosed as SEQ ID NO: 61), D411TQNAPR (“TQNAPR” disclosed as SEQ ID NO: 62), d411, D411TENAPR (“TENAPR” disclosed as SEQ ID NO: 63), D411TQNAPN (“TQNAPN” disclosed as SEQ ID NO: 64), d412, E412Q, d413, A413N, A413NNPN (“NNPN” disclosed as SEQ ID NO: 65), d414, I414A, I414S, I414N, d415, K415P, d416, Q416D, Q416R, Q416N, Q416E, T418P, L419G, P420E, L424M, L424V, W425V, W425L, I426M, I426L, Q430T, Q430N, Q430S, V431I, V431L, M432T, M432F, D433S, D433K, H436W, N442T, E443R, E443K, E443RASFA (“RASFA” disclosed as SEQ ID NO: 66), d444, d445, C445M, C445T, C445S, C445A, d446, d447, V447A, D455S, D455G, Y459G, Y459E, Y459D, R461D, R461G, H462R, H462G, F464L, F464Y, R465H, V469M, A470C, A470G, L471Y, L471K, L471M, L471W, L471I, L471R, V472I, V472F, V472C, V472Y, V472L, V472M, K474E, K474EDGH (“EDGH” disclosed as SEQ ID NO: 67), K474EDGY (“EDGY” disclosed as SEQ ID NO: 68), Q475D, d475, d476, d477, R477H, R477F, R477N, R477E, R477Y, Y478W, Y478I, Y478M, M479T, M479H, M479Y, M479F, M479W, I480V, I480L, V481C, V481I, V481T, T484L, E486D, K489I, S490R, S490C, S490V, Q491G, Q491R, Q491N, K493F, K493H, K493Y, K493L, Q494N, A496F, A496S, A496Y, M503L, M503V, M503I, G506R, G506RHPD (“RHPD” disclosed as SEQ ID NO: 69), d507, d508, A508P, d509, Q509D, T513A, T513V, T513C, I518V, Q519P, V521E, V521P, K523Q, K523A, K523R, K523M, K523Y, K523D, G524S, G524A, N525S, N525T, N525D, N525G, N525E, A527P, A527T, A527V, P528A, P528V, R529V, R529A, Y539L, Y539V, D540T, V542E, V542D, T546A, T546K, Q551K, Q551R, P553S, K554S, K554P, K554H, Y555F, Q557K, H559P, A560G, I562V, d562, V564F, I565V, I565F, I565FV, D566E, D566VE, A583Q, A583E, or any combination thereof, in reference to SEQ ID NO: 1. It is understood that when multiple alterations without any delimitating characters (e.g., commas and/or spaces) are used in reference to an alteration at a specific position, the alteration is conjunctive and all identified alterations alter the corresponding specific position. For example, a G506RHPD (“RHPD” disclosed as SEQ ID NO: 69) means that R replaces the G at position 506 and HPD are inserted adjacent to the R. It is also understood that when a lower case “d” is used in reference to an alteration at a specific position, the alteration is a deletion at the specific position. For example the alteration d475 means that the residue at position 475 has been deleted.

In some embodiments, an engineered ACL described herein has one or more amino acid alterations comprising: N7K, N7S, A8S, I10W, I10Y, I10F, N12E, N12P, N12D, V15I, D16E, Y19F, A52R, A52K, M54L, L64A, S66R, S67A, R73L, H74K, S82T, L87I, F90L, F90M, V94T, V100L, I111V, S115N, G117S, S119T, Q121D, V129I, T131G, A138D, A138S, K139E, A140K, A140E, A140D, V144T, K150L, K150I, A165S, L166F, M172L, M172A, M172D, R182Q, S191A, G199A, S209P, V212C, S217R, S221T, S221A, S223N, S223I, S223A, S223L, S223H, E224Q, N227E, N227T, N227A, A229I, A229L, A229M, S230Q, S230A, E231H, E231Q, E231P, S232Y, S232K, S232Q, S232N, L233W, L233Y, A234Y, A234F, A234H, T235S, Q238D, Q238H, Q238T, A240T, A240H, A240C, A240S, I243C, I243M, L249M, L249V, S253H, S253M, S253Q, K254M, L256I, L256V, G258T, G258H, G258A, G258L, G258M, N259A, A262K, A262C, A262N, Y268F, G271R, G271Q, I275E, V277L, V277M, Q281V, S288V, S288I, S288F, S288T, S288H, S288Y, T290V, T290A, I296L, I296M, V297I, T300A, I304E, E306D, M316L, A318S, A318R, A318H, A318W, A318Y, A318M, A318T, A318C, A318Q, A318V, A318I, A318L, L320M, L320F, L320T, L320Y, L320W, S321C, A323G, A328V, A328H, A328R, V330I, V330L, T331I, T336K, R352K, I353V, I353L, S363G, P364S, V365C, V365L, V365I, V365M, A366V, F369H, F369S, F369T, F369M, E372S, E372K, E372H, E372N, K374Q, K374R, K374T, K374P, K374H, K374S, K374A, K374D, K374E, R376T, I378A, I378P, I378S, I378V, S388T, V390C, V390A, N394D, V398N, D406L, P407D, Q416N, Q416E, M432F, E443R, E443K, C445A, D455S, D455G, H462R, V469M, A470G, L471R, L471M, L471W, I480V, V481I, V481C, T484L, E486D, S490C, Q491R, Q491G, K493L, K493H, K493Y, Q494N, A496Y, A508P, T513A, T513V, T513C, I518V, Q519P, V521E, V521P, K523R, K523Q, K523A, K523Y, K523M, K523D, N525D, N525S, N525T, N525G, N525E, T546K, K554H, K554S, K554P, Y555F, A560G, I565V, A583Q, or A583E, in reference to SEQ ID NO: 1.

In some embodiments, an engineered ACL described herein has one or more amino acid alterations comprising: I10F, G199A, S223L, S223H, S223A, A229M, S230Q, S230A, E231P, S232N, L233W, L233Y, A234H, T235S, Q238T, I243C, I243M, L256I, L256V, G258L, G258M, V277M, S288Y, S288H, T300A, A318V, A318T, A318C, A318I, A318L, A318Q, A318M, A318Y, L320W, L320Y, S321C, A323G, A328R, V330L, P364S, V365I, V365M, F369M, E372N, K374E, I378V, H462R, V469M, I480V, V481C, T484L, T513C, T513A, T513V, K523D, N525G, N525E, N525T, K554S, K554P, A560G, or A583E, in reference to SEQ ID NO: 1.

In some embodiments, an engineered ACL described herein has one or more amino acid alterations comprising: L233W, P364S, S232N, L320W, A318V, S321C, S223L, L320Y, T513C, Q238T, L233Y, K523D, A318T, S288Y, A318C, A234H, A323G, T513A, S288H, S223H, A318I, A318L, or G258L in reference to SEQ ID NO: 1.

In some embodiments, an engineered ACL described herein has one or more amino acid alterations comprising: D5R, D5E, A6L, S7K, S7T, G8P, E10K, E10D, E10P, E10N, E10R, L12A, L12I, L12V, L12M, P13C, H14P, H14A, H14Q, D15E, D15L, D15S, D15R, V16C, V16L, V161, E19D, E19R, S20H, S20K, S20R, S20D, P22D, P22E, S25N, L28M, V37C, P39R, N40T, T41M, T41L, T41V, T41I, A43S, W44M, L45I, L45V, W48P, K50I, K50F, K50R, K50V, K50L, E51R, P53L, A57F, I58V, I58L, I58M, I58F, A59V, R61P, R61E, A62D, G63D, W66G, R67K, H69F, H69Y, H69L, Y71F, Y71W, N72A, N72R, L74F, L75D, N76T, N76D, N76A, N76R, M77R, V78A, N80K, V81C, G83S, G83H, G83Q, G83N, A84Y, A84H, L85F, L86A, L86Q, A87R, R88L, A92K, D93G, S94R, P95R, V97A, V98M, M99L, S100C, G101P, G103R, V104L, V104W, V104A, H106W, H106F, A107V, A107P, A107I, A107L, A107W, A107F, I108V, L109V, S110M, S110F, S110Y, S110A, SHOD, A113T, A113C, A113V, Q114M, Q114W, Q114L, Q114G, Q114A, Y115A, V116I, P119V, P119T, P1191, I120M, I120F, I120V, I120A, I120L, I120W, I120Y, V121T, P122A, L123M, L123V, L123I, A124Y, A124S, A124N, E125P, E125T, E125W, Q126M, Q126N, Q126R, Q126A, Y127F, Y127W, Y127L, S128R, I130M, P131E, R136E, R136Q, R136K, L137M, V140M, V140C, V140I, L141M, E142N, E142Q, K145G, K145R, K145D, A147S, M148L, M148V, M148A, A149V, A149L, F150V, V151C, Y157L, S159R, S159E, L161I, L163V, P164A, Q165R, E167R, G168H, V169I, E170I, E170V, E170L, A173V, K175R, T176D, D177E, V181E, V183I, A185S, F186Y, A187D, L189V, L190V, G191A, G191E, H192T, G193A, Q194S, G195S, G196P, H201A, H201P, H201L, G205T, T208D, L209I, L209T, L209P, L209V, K211L, K211T, K211C, K211M, K211V, K211A, I212L, I212M, L213I, L213C, L213V, L213Q, L213M, F214Y, T215S, S216G, S218T, S219T, S220G, D221T, D221M, D221L, D221R, D221K, D221H, P222S, G224A, V225A, L226M, L226I, L226V, T227L, T227D, T227H, T227I, T228S, R230H, R230K, M231N, M232L, M232I, M232V, C233L, C233V, C233M, C233W, C233A, C233I, A234S, A234W, N235Q, N235S, Q236A, Q236C, Q236I, Q236M, Q236V, T237A, T237M, T237Q, T237R, T237E, Q238M, Q238S, Q238W, Q238N, Q238H, I239F, I239M, I239V, I239C, I239L, S240A, S240R, D241A, D241H, D241E, D241Q, A242V, A242C, A242T, A242W, L243I, L243F, L243M, L243W, L243V, R244E, R244P, R244Q, R244H, R244G, F245M, F245L, F245H, F245W, F245Y, L246I, L246T, L246M, K247R, K247P, K247E, P251E, R252H, R252V, I253Y, I253M, I253F, I253L, I253V, L254I, L254F, L254V, L254M, D255A, D255T, D255N, D255L, D255I, D255G, D255H, D255C, D255V, D255S, W256F, W256V, W256I, W256C, W256Y, L257M, W259L, W259F, N260H, N260S, N260Y, N260F, V262C, V262T, V262A, V262I, V262M, F263A, F263Y, F263H, G264A, G265M, G265L, G265W, G265F, G265C, S266N, S266T, S266M, S266V, H267I, H267G, H267M, H267W, H267F, H267L, H267V, H267C, H267A, N268M, N268A, N268C, N268G, N268V, F269L, F269M, F269I, F269V, N270M, N270H, N270Y, N270T, M271A, M271P, M271C, M271T, M271L, M272C, M272V, M272P, M272A, M272T, L273M, L273V, L273F, L273I, A274Y, A274R, A274V, A274M, A274C, A274H, A274W, N275G, N275C, N275S, N275H, G277A, G277C, S278C, S278T, S278A, S278H, L279V, L279M, L279I, I281F, I281L, I281V, I281M, D282P, D282M, D282E, D282A, D282H, N283E, N283D, N283P, N283R, G284F, G284R, G284W, K285R, K285F, K285W, K285D, P286D, P286F, T287P, T287A, T287V, G290A, F291M, F291V, F291L, F291P, F291I, E292H, T294I, T294L, T294A, L295W, L295M, L295V, L295I, R296D, R296Q, I298M, I298V, I298L, K299A, E300Q, P302Q, G303V, G303I, G303A, G303P, G303C, T304S, L305W, L305Y, L305V, L305I, L305F, L305H, L305M, A306V, A306Y, A306I, A306M, A306L, A306F, F307P, F307W, F307Y, F307H, F307M, N308T, N308A, N308S, N308L, N308G, N308V, N308M, V311A, V311R, V311P, V311T, G312I, G312M, G312V, G312F, Y313L, Y313F, Y313W, A314Q, A314E, A314H, M315R, M315A, M315K, M315L, L316W, L316M, L316I, L316F, N318H, N318D, E321R, E321Q, R322Q, R322K, R322A, R322H, Y330L, Y330F, F331G, E333S, L334I, L334M, L334V, D335R, D335K, L336T, L336I, L336M, L336Y, L336V, L336F, L336W, I337L, I337M, I337F, I337A, I337V, F338W, F338L, F338M, F338V, Y339F, Y339T, Y339S, Y339C, A340G, G341A, G341S, A342S, S343P, S343A, L344M, L344I, L344V, L344C, P345S, Q346V, Q346A, Q346P, Q346E, D347E, V348T, V348I, V348L, W349M, W349F, W349L, W349A, W349I, W349Y, S351F, S351R, S351Q, S351W, L352I, L352M, L352Y, E353R, R354K, F355A, F355R, M357W, M357G, M357V, M357L, M357P, M357I, M357F, M357A, E358Q, E359H, E359Q, G361F, S362H, S362L, S362C, L363D, L363V, L363I, L363G, L365M, L365V, L365I, M366I, M366V, M366L, M366F, A367T, A367C, A367M, A367I, A367V, A367L, S368N, S368Q, S368E, S368T, S369G, S369A, W370Y, W370F, M372L, M372Q, T373S, T375A, T375C, A376T, A376S, A376C, A376G, P377G, P377M, A378C, A378T, A378V, T379C, T379V, T379I, T379L, T379A, I380F, I380C, I380H, I380S, I380M, I380T, I380V, M381N, M381F, M381L, M381C, M381V, V382L, V382N, V382C, V382T, V382P, H383D, H383N, H383R, H383P, H383Y, E384P, E384N, E384D, E384W, P385G, P385D, I386D, I386V, I386L, I386T, G387D, G387R, G387N, G387H, G387E, R388K, R388H, S389A, S389P, S389L, V391S, V391T, I392V, I392C, V394R, V394K, V394I, V394Q, V394L, P395A, L396F, L396I, L396M, L396V, G398D, G398H, G398N, A399M, A399V, A399T, A399C, A399I, E400D, E400Q, V401M, V401I, V401C, V401L, V401A, K402R, L404V, P405D, D406E, D407P, M409Q, M409E, M409G, C411D, C411S, C411V, C411G, C411N, L413I, L413V, R414W, R414L, R414V, R414C, R414Q, V415I, K416R, G417S, P418D, N419H, N419S, N419Q, N419M, I420V, M421T, M421F, F425H, F425L, K430A, A432K, E433R, E433D, A434T, A434V, F435I, F435M, F435L, L441F, L441M, L441Y, V442K, V442H, V442Y, V442R, V442C, T443S, A446M, A446I, A446L, A446V, V447A, V447G, F449I, V450D, V450M, D451E, D452P, A453G, P455E, P455G, P455R, S456D, S456E, S456Q, S456N, G458N, G458F, G458Y, G458W, L459Y, V460Y, V460F, V460I, V460R, V460W, F461I, F461L, F461Y, D462I, D462T, D462V, G463D, R464D, V465L, S466D, S466K, S466A, E467K, D468M, F469L, K470V, L471T, L471V, T473S, T473N, G474E, W476N, W476Y, W476L, W476F, W476V, W476I, Q478T, Q478L, A479P, A479V, G480L, G480I, G480S, G480P, G480A, G480V, V481P, V481E, R483E, D485A, D485L, D485E, D485R, L490C, G492P, L493F, D496H, V497A, V498C, H502P, R504H, I507Y, I507V, I507W, G508V, F510A, I511V, I511L, F512I, P513V, L514N, P515D, P515S, G516A, V518I, S519T, T521P, T521W, V526L, V526M, I527L, S532R, S532H, I535L, R540Q, M542Y, M542W, A546N, R552Q, N554E, I558F, S560D, E561D, P563F, P563L, P563W, Q564D, Q564K, Q564S, Q564L, Q564T, Q564R, L565M, L565I, L565V, L565P, L565T, K566A, K566E, K566D, K566P, D567K, D567Q, D567T, D567N, D567S, D567A, H568G, H568N, H568D, E569M, E569K, I570V, I570L, D572R, D572P, K573T, K573S, G574M, G574L, S575A, S575Y, S575K, S575T, N577Y, N577A, N577L, I578Q, I578A, T583D, R584N, R585H, R585L, D587A, L588Q, R591Q, R591E, Y593A, N595E, E596K, P598D, A599S, I601L or any combination thereof, in reference to SEQ ID NO: 2.

In some embodiments, an engineered ACL described herein has one or more amino acid alterations comprising: D5E, D5R, A6L, S7K, S7T, G8P, E10D, E10N, E10R, E10P, E10K, L12A, L12V, L12M, L12I, P13C, H14Q, H14P, H14A, D15S, D15E, V16I, V16C, E19D, E19R, S20H, S20K, S20R, S20D, P22E, P22D, S25N, L28M, V37C, P39R, N40T, T41V, T41I, T41M, A43S, L45I, L45V, K50R, K50L, K50I, K50V, K50F, E51R, P53L, A57F, I58F, I58M, I58L, I58V, A59V, R61P, A62D, G63D, R67K, H69F, H69L, H69Y, Y71W, Y71F, N72A, N72R, L74F, L75D, N76D, N76T, N76A, N76R, M77R, V78A, N80K, V81C, G83S, G83N, A84Y, A84H, L85F, L86Q, L86A, A87R, R88L, A92K, D93G, S94R, P95R, V97A, V98M, M99L, S100C, G101P, G103R, V104A, V104L, V104W, H106F, I108V, L109V, S110A, S110M, A113C, V116I, P119T, P119V, P119I, I120Y, I120V, I120F, I120L, I120M, I120A, V121T, L123I, L123M, L123V, A124S, A124N, A124Y, E125P, E125T, E125W, Q126R, Q126A, Q126N, Q126M, Y127L, Y127W, Y127F, S128R, I130M, P131E, R136E, R136K, R136Q, L137M, V140M, V140I, V140C, L141M, E142Q, E142N, K145R, K145D, K145G, A147S, M148V, M148L, M148A, A149V, F150V, V151C, S159E, S159R, L161I, L163V, P164A, Q165R, E167R, G168H, V169I, E170I, E170L, E170V, A173V, K175R, T176D, D177E, V181E, V183I, A185S, F186Y, A187D, L189V, L190V, G191E, G191A, H192T, G193A, Q194S, G195S, G196P, H201L, H201A, G205T, T208D, L209I, L209T, L209V, K211L, I212L, I212M, L213I, L213M, L213V, F214Y, T215S, S218T, S219T, S220G, D221K, D221L, D221T, D221H, D221R, D221M, P222S, G224A, L226M, L226V, L226I, T227I, T228S, R230H, R230K, M232L, C233A, A234S, N235Q, Q236M, T237A, T237M, T237Q, Q238M, Q238H, I239V, I239L, I239M, S240A, D241Q, D241E, D241H, D241A, A242C, A242V, A242T, L243W, L243F, L243M, L243I, L243V, R244P, R244Q, R244G, R244H, R244E, F245L, F245M, F245W, L246I, K247R, K247P, R252V, R252H, I253L, I253V, I253M, L254M, L254I, L254V, D255C, D255V, D255T, D255N, W256Y, W256V, W256F, N260S, N260H, N260F, N260Y, V262T, V262C, V262A, F263Y, G264A, G265M, S266N, S266M, H267M, H267F, H267L, H267W, H267G, N268M, N268A, N270M, M271L, M271C, M271A, M272A, M272V, M272T, M272P, M272C, L273I, L273V, L273M, L273F, A274C, A274M, A274H, A274R, A274Y, N275H, N275C, G277A, S278T, S278A, S278C, L279M, L279I, L279V, I281V, I281L, N283E, N283D, N283P, N283R, K285R, K285F, K285D, P286D, P286F, T287A, T287P, T287V, G290A, F291M, F291I, F291L, F291V, E292H, T294A, L295V, L295I, L295M, R296Q, R296D, I298M, I298V, I298L, K299A, E300Q, P302Q, G303A, G303P, G303C, G303V, T304S, L305V, L305M, L305I, A306I, A306V, A306Y, F307M, F307W, F307P, N308G, V311T, V311A, V311R, G312F, G312V, G312M, Y313F, Y313W, A314Q, A314H, A314E, M315A, M315K, M315L, M315R, L316I, L316M, N318D, N318H, E321R, E321Q, R322A, R322K, R322H, R322Q, Y330F, F331G, E333S, L334V, L334I, L334M, D335K, L336V, L336T, L336M, L336Y, L336W, L336I, L336F, I337L, I337V, I337M, F338L, F338M, F338V, F338W, Y339T, Y339S, Y339F, A340G, A342S, S343P, S343A, L344I, L344C, L344M, L344V, P345S, Q346E, Q346V, Q346A, Q346P, D347E, V348I, V348L, V348T, W349Y, W349F, W349L, S351R, S351Q, S351W, L352I, L352M, E353R, R354K, F355A, M357F, M357I, M357V, M357A, M357G, M357L, M357W, E358Q, E359Q, S362C, S362H, S362L, L363V, L363I, L363G, L365M, L365I, L365V, M366I, M366L, M366F, M366V, A367V, A367I, A367L, A367M, A367T, A367C, S368N, S369A, S369G, W370Y, T375C, T375A, A376G, A376S, P377G, A378T, T379V, T379C, T379L, T379A, T379I, 1380S, I380V, I380H, I380T, M381L, M381C, V382T, V382C, V382N, V382L, V382P, H383Y, H383R, E384N, E384W, E384D, P385G, I386V, I386T, I386L, G387R, G387H, G387N, G387D, G387E, R388K, R388H, S389A, S389P, S389L, V391S, V391T, I392V, V394I, V394K, L396V, G398H, A399C, A399I, A399T, A399V, E400Q, E400D, V401L, V401I, V401C, V401A, V401M, L404V, D406E, D407P, M409G, M409Q, M409E, C411S, R414L, R414C, K416R, N419Q, N419H, N419M, N419S, I420V, M421F, M421T, F425L, F425H, K430A, A432K, E433R, E433D, A434V, A434T, F435M, L441F, L441M, V442Y, V442R, V442K, V442C, V442H, T443S, A446L, A446V, A446M, A446I, V447A, V450M, V450D, D451E, D452P, A453G, S456N, S456D, V460F, V460I, V460R, V460W, V460Y, F461Y, F461L, D462V, D462I, G463D, V465L, S466A, S466D, S466K, E467K, D468M, F469L, K470V, L471T, L471V, T473N, T473S, W476Y, W476F, W476V, W476L, W476I, W476N, Q478T, A479V, A479P, G480S, G480A, G480P, G480L, V481P, V481E, D485L, D485E, D485R, D485A, L490C, G492P, V498C, 1507V, G508V, F510A, I511V, I511L, F512I, L514N, P515D, P515S, G516A, V518I, S519T, T521P, T521W, V526M, V526L, 1527L, S532R, S532H, 1535L, R540Q, M542Y, M542W, A546N, R552Q, N554E, S560D, E561D, P563L, P563F, Q564S, Q564T, Q564L, Q564D, Q564R, Q564K, L565T, L565M, L565P, L565V, L565I, K566D, K566A, K566E, K566P, D567A, D567S, D567Q, D567N, D567T, D567K, H568G, H568D, H568N, E569M, E569K, 1570V, 1570L, D572P, K573S, K573T, G574M, G574L, S575T, S575Y, N577A, N577L, 1578A, 1578Q, T583D, R584N, R585H, D587A, L588Q, R591Q, R591E, Y593A, N595E, E596K, P598D, A599S, or 1601L, in reference to SEQ ID NO: 2.

In some embodiments, an engineered ACL described herein has one or more amino acid alterations comprising: D5E, D5R, A6L, S7K, S7T, G8P, E10D, E10N, E10R, E10P, L12A, L12V, L12M, H14Q, H14P, D15S, D15E, V161, E19D, E19R, S20H, S20K, S20R, S20D, P22E, P22D, L28M, P39R, T41V, A43S, L451, K50R, K50L, K501, K50V, K50F, A57F, 158F, 158M, 158L, 158V, R61P, A62D, G63D, R67K, H69F, H69L, H69Y, Y71W, Y71F, N72A, N72R, N76D, N76T, N76A, N76R, M77R, V78A, V81C, A84Y, L85F, L86Q, A87R, A92K, D93G, S94R, P95R, V98M, V104A, 1108V, L109V, A113C, V116I, P119T, 1120Y, I120V, I120F, I120L, I120M, I120A, L1231, A124S, A124N, E125P, E125T, E125W, Q126R, Q126A, Q126N, Y127L, Y127W, 1130M, P131E, R136E, R136K, V140M, V1401, V140C, L141M, E142Q, E142N, K145R, A147S, M148V, A149V, V151C, S159E, S159R, L161I, L163V, P164A, E167R, G168H, V1691, E1701, E170L, E170V, A173V, K175R, T176D, D177E, V181E, V1831, A185S, F186Y, A187D, L190V, G191E, G191A, G193A, Q194S, G195S, H201L, H201A, G205T, L2091, F214Y, S218T, S219T, S220G, D221K, D221L, D221T, D221H, L226M, L226V, T228S, R230H, R230K, M232L, A234S, T237A, Q238M, 1239V, D241Q, D241E, A242C, A242V, A242T, L243W, L243F, L243M, R244P, R244Q, L246I, K247R, 1253L, 1253V, L254M, L2541, L254V, N260S, N260H, V262T, F263Y, S266N, H267M, H267F, M272A, L273I, L273V, L273M, L273F, A274C, S278T, S278A, L279M, I281V, N283E, K285R, G290A, F291M, F291I, F291L, F291V, E292H, L295V, L295I, L295M, R296Q, 1298M, 1298V, E300Q, P302Q, G303A, G303P, T304S, L305V, L305M, L305I, F307M, V311T, V311A, Y313F, A314Q, A314H, M315A, M315K, M315L, M315R, L3161, N318D, N318H, E321R, E321Q, R322A, R322K, R322H, R322Q, Y330F, E333S, L334V, L3341, L336V, L336T, L336M, L336Y, 1337L, 1337V, F338L, F338M, F338V, Y339T, A340G, L344I, L344C, L344M, P345S, Q346E, D347E, V348I, V348L, V348T, W349Y, W349F, S351R, S351Q, L352I, E353R, R354K, F355A, M357F, M357I, M357V, M357A, M357G, M357L, M357W, E358Q, E359Q, S362C, S362H, L363V, L363I, L365M, A367V, A367I, A367L, A367M, S369A, T375C, A376G, A376S, T379V, T379C, T379L, V382T, V382C, H383Y, E384N, 1386V, 1386T, G387R, G387H, G387N, G387D, R388K, R388H, S389A, S389P, S389L, A399C, A399I, A399T, A399V, E400Q, V401L, V401I, V401C, L404V, D407P, M409G, K416R, N419Q, 1420V, M421F, K430A, A432K, E433R, E433D, A434V, L441F, V442Y, V442R, V442K, V442C, V442H, A446L, A446V, A446M, A446I, V450M, D452P, A453G, S456N, S456D, V460F, V460I, V460R, F461Y, V465L, S466A, S466D, L471T, T473N, T473S, W476Y, W476F, W476V, W476L, W476I, A479V, A479P, G480S, G480A, G480P, V481P, V481E, D485L, D485E, D485R, D485A, L490C, G492P, I507V, I511V, L514N, P515D, P515S, G516A, V518I, S519T, T521P, T521W, V526M, V526L, I527L, S532R, S532H, R540Q, M542Y, A546N, N554E, E561D, Q564S, Q564T, L565T, L565M, L565P, L565V, L565I, K566D, D567A, D567S, D567Q, D567N, H568G, H568D, H568N, I570V, D572P, G574M, S575T, N577A, R584N, D587A, N595E, E596K, or A599S, in reference to SEQ ID NO: 2.

In some embodiments, an engineered ACL described herein has one or more amino acid alterations comprising: D5E, D5R, A6L, S7K, S7T, G8P, E10D, E10N, L12A, H14Q, H14P, D15S, D15E, E19D, S20H, S20K, S20R, P22E, T41V, L45I, K50R, K50L, K50I, K50V, I58F, I58M, I58L, A62D, G63D, R67K, H69F, H69L, H69Y, N72A, N72R, N76D, N76T, N76A, N76R, M77R, V81C, L86Q, A87R, A92K, D93G, S94R, P95R, V98M, V104A, 1108V, A113C, V116I, I120Y, I120V, I120F, I120L, L123I, A124S, E125P, E125T, Q126R, Q126A, Q126N, Y127L, I130M, R136E, V140M, V140I, L141M, E142Q, E142N, K145R, A147S, M148V, A149V, V151C, S159E, L161I, L163V, P164A, E167R, G168H, V169I, K175R, T176D, D177E, A185S, F186Y, A187D, L190V, G191E, G191A, G193A, G195S, H201L, H201A, G205T, L209I, F214Y, S219T, S220G, D221K, T228S, R230H, R230K, M232L, A234S, T237A, Q238M, I239V, D241Q, D241E, A242C, A242V, A242T, L243W, L243F, L243M, R244P, R244Q, L246I, K247R, I253L, L254M, N260S, N260H, F263Y, S266N, H267M, H267F, M272A, L273I, A274C, S278T, S278A, L279M, I281V, N283E, F291M, F291I, F291L, F291V, E292H, L295V, L295I, L295M, I298M, I298V, E300Q, P302Q, G303A, G303P, T304S, L305V, F307M, V311T, V311A, Y313F, A314Q, A314H, M315A, M315K, N318D, E321R, E321Q, R322A, R322K, R322H, R322Q, Y330F, E333S, L334V, L334I, L336V, L336T, L336M, I337L, I337V, F338L, F338M, A340G, L344I, V348I, V348L, W349Y, S351R, S351Q, E353R, R354K, F355A, M357F, M357I, E358Q, E359Q, S362C, S362H, L363V, L365M, A367V, A367I, A367L, A367M, S369A, A376G, T379V, T379C, V382T, V382C, H383Y, I386V, I386T, G387R, G387H, G387N, R388K, S389A, S389P, A399C, A399I, A399T, A399V, E400Q, V401L, V401I, L404V, D407P, M409G, I420V, M421F, K430A, A432K, E433R, E433D, A434V, V442Y, V442R, V442K, V442C, V442H, A446L, A446V, A446M, A446I, V450M, D452P, A453G, S456N, S456D, V460F, V460I, F461Y, V465L, S466A, T473N, T473S, W476Y, W476F, W476V, W476L, W476I, A479V, A479P, G480S, G480A, V481P, V481E, D485L, D485E, D485R, D485A, G492P, I507V, L514N, P515D, P515S, G516A, V518I, S519T, T521P, V526M, V526L, I527L, S532R, S532H, R540Q, M542Y, N554E, Q564S, Q564T, L565T, L565M, L565P, L565V, L565I, D567A, D567S, D567Q, D567N, H568G, H568D, I570V, D572P, S575T, R584N, D587A, N595E, or E596K, in reference to SEQ ID NO: 2.

In some embodiments, an engineered ACL provided herein has an amino acid sequence that is a variant of SEQ ID NO: 1 that includes one or more alterations as described in TABLE 8, wherein the portion, other than the one or more alterations described in TABLE 8, of the engineered ACL has at least 65%, 70%, 75%, 80%, 85%, 90%, 95%, 98% or 99% sequence identity, or is identical, to an amino acid sequence referenced as SEQ ID NO: 1. Accordingly, in some embodiments, an engineered ACL provided herein has an amino acid sequence that includes one or more alterations as described in TABLE 8 and the portion, other than the alteration described in TABLE 8, of the engineered ACL has at least 65% identical to SEQ ID NO: 1. In some embodiments, an engineered ACL provided herein has an amino acid sequence that includes one or more alterations as described in TABLE 8 and the portion, other than the alteration described in TABLE 8, of the engineered ACL has at least 70% identical to SEQ ID NO: 1. In some embodiments, an engineered ACL provided herein has an amino acid sequence that includes one or more alterations as described in TABLE 8 and the portion, other than the alteration described in TABLE 8, of the engineered ACL has at least 75% identical to SEQ ID NO: 1. In some embodiments, an engineered ACL provided herein has an amino acid sequence that includes one or more alterations as described in TABLE 8 and the portion, other than the alteration described in TABLE 8, of the engineered ACL has at least 80% identical to SEQ ID NO: 1. In some embodiments, an engineered ACL provided herein has an amino acid sequence that includes one or more alterations as described in TABLE 8 and the portion, other than the alteration described in TABLE 8, of the engineered ACL has at least 85% identical to SEQ ID NO: 1. In some embodiments, an engineered ACL provided herein has an amino acid sequence that includes one or more alterations as described in TABLE 8 and the portion, other than the alteration described in TABLE 8, of the engineered ACL has at least 90% identical to SEQ ID NO: 1. In some embodiments, an engineered ACL provided herein has an amino acid sequence that includes one or more alterations as described in TABLE 8 and the portion, other than the alteration described in TABLE 8, of the engineered ACL has at least 95% identical to SEQ ID NO: 1. In some embodiments, an engineered ACL provided herein has an amino acid sequence that includes one or more alterations as described in TABLE 8 and the portion, other than the alteration described in TABLE 8, of the engineered ACL has at least 98% identical to SEQ ID NO: 1. In some embodiments, an engineered ACL provided herein has an amino acid sequence that includes one or more alterations as described in TABLE 8 and the portion, other than the alteration described in TABLE 8, of the engineered ACL has at least 99% identical to SEQ ID NO: 1. In some embodiments, an engineered ACL provided herein has an amino acid sequence that includes one or more alterations as described in TABLE 8 and the portion, other than the alteration described in TABLE 8, of the engineered ACL is identical to SEQ ID NO: 1.

In some embodiments, an engineered ACL provided herein has an amino acid sequence that is a variant of SEQ ID NO: 1 that includes one or more alterations as described in TABLE 8 and/or one or more, or a combination of alterations, as described in TABLE 9, wherein the portion, other than the one or more alterations described in TABLE 8 or the one or more, or combination of alterations, as described in TABLE 9, of the engineered ACL has at least 65%, 70%, 75%, 80%, 85%, 90%, 95%, 98% or 99% sequence identity, or is identical, to an amino acid sequence referenced as SEQ ID NO: 1. Accordingly, in some embodiments, an engineered ACL provided herein has an amino acid sequence that includes one or more alterations as described in TABLE 8 and/or one or more, or a combination of alterations, as described in TABLE 9 and the portion, other than the alteration described in TABLE 8 or the one or more, or combination of alterations, as described in TABLE 9, of the engineered ACL has at least 65% sequence identity to SEQ ID NO: 1. In some embodiments, an engineered ACL provided herein has an amino acid sequence that includes one or more alterations as described in TABLE 8 and/or one or more, or a combination of alterations, as described in TABLE 9 and the portion, other than the alteration described in TABLE 8 or the one or more, or combination of alterations, as described in TABLE 9, of the engineered ACL has at least 70% sequence identity to SEQ ID NO: 1. In some embodiments, an engineered ACL provided herein has an amino acid sequence that includes one or more alterations as described in TABLE 8 and/or one or more, or a combination of alterations, as described in TABLE 9 and the portion, other than the alteration described in TABLE 8 or the one or more, or combination of alterations, as described in TABLE 9, of the engineered ACL has at least 75% sequence identity to SEQ ID NO: 1. In some embodiments, an engineered ACL provided herein has an amino acid sequence that includes one or more alterations as described in TABLE 8 and/or one or more, or a combination of alterations, as described in TABLE 9 and the portion, other than the alteration described in TABLE 8 or the one or more, or combination of alterations, as described in TABLE 9, of the engineered ACL has at least 80% sequence identity to SEQ ID NO: 1. In some embodiments, an engineered ACL provided herein has an amino acid sequence that includes one or more alterations as described in TABLE 8 and/or one or more, or a combination of alterations, as described in TABLE 9 and the portion, other than the alteration described in TABLE 8 or the one or more, or combination of alterations, as described in TABLE 9, of the engineered ACL has at least 85% sequence identity to SEQ ID NO: 1. In some embodiments, an engineered ACL provided herein has an amino acid sequence that includes one or more alterations as described in TABLE 8 and/or one or more, or a combination of alterations, as described in TABLE 9 and the portion, other than the alteration described in TABLE 8 or the one or more, or combination of alterations, as described in TABLE 9, of the engineered ACL has at least 90% sequence identity to SEQ ID NO: 1. In some embodiments, an engineered ACL provided herein has an amino acid sequence that includes one or more alterations as described in TABLE 8 and/or one or more, or a combination of alterations, as described in TABLE 9 and the portion, other than the alteration described in TABLE 8 or the one or more, or combination of alterations, as described in TABLE 9, of the engineered ACL has at least 95% sequence identity to SEQ ID NO: 1. In some embodiments, an engineered ACL provided herein has an amino acid sequence that includes one or more alterations as described in TABLE 8 and/or one or more, or a combination of alterations, as described in TABLE 9 and the portion, other than the alteration described in TABLE 8 or the one or more, or combination of alterations, as described in TABLE 9, of the engineered ACL has at least 98% sequence identity to SEQ ID NO: 1. In some embodiments, an engineered ACL provided herein has an amino acid sequence that includes one or more alterations as described in TABLE 8 and/or one or more, or a combination of alterations, as described in TABLE 9 and the portion, other than the alteration described in TABLE 8 or the one or more, or combination of alterations, as described in TABLE 9, of the engineered ACL has at least 99% sequence identity to SEQ ID NO: 1. In some embodiments, an engineered ACL provided herein has an amino acid sequence that includes one or more alterations as described in TABLE 8 and/or one or more, or a combination of alterations, as described in TABLE 9 and the portion, other than the alteration described in TABLE 8 or the one or more, or combination of alterations, as described in TABLE 9, of the engineered ACL is identical to SEQ ID NO: 1.

In some embodiments, an engineered ACL provided herein has an amino acid sequence that is a variant of SEQ ID NO: 2 that includes one or more alterations as described in TABLE 9, wherein the portion, other than the one or more alterations described in TABLE 9, of the engineered ACL has at least 65%, 70%, 75%, 80%, 85%, 90%, 95%, 98% or 99% sequence identity, or is identical, to an amino acid sequence referenced as SEQ ID NO: 2. Accordingly, in some embodiments, an engineered ACL provided herein has an amino acid sequence that includes one or more alterations as described in TABLE 9 and the portion, other than the alteration described in TABLE 9, of the engineered ACL has at least 65% identical to SEQ ID NO: 2. In some embodiments, an engineered ACL provided herein has an amino acid sequence that includes one or more alterations as described in TABLE 9 and the portion, other than the alteration described in TABLE 9, of the engineered ACL has at least 70% identical to SEQ ID NO: 2. In some embodiments, an engineered ACL provided herein has an amino acid sequence that includes one or more alterations as described in TABLE 9 and the portion, other than the alteration described in TABLE 9, of the engineered ACL has at least 75% identical to SEQ ID NO: 2. In some embodiments, an engineered ACL provided herein has an amino acid sequence that includes one or more alterations as described in TABLE 9 and the portion, other than the alteration described in TABLE 9, of the engineered ACL has at least 80% identical to SEQ ID NO: 2. In some embodiments, an engineered ACL provided herein has an amino acid sequence that includes one or more alterations as described in TABLE 9 and the portion, other than the alteration described in TABLE 9, of the engineered ACL has at least 85% identical to SEQ ID NO: 2. In some embodiments, an engineered ACL provided herein has an amino acid sequence that includes one or more alterations as described in TABLE 9 and the portion, other than the alteration described in TABLE 9, of the engineered ACL has at least 90% identical to SEQ ID NO: 2. In some embodiments, an engineered ACL provided herein has an amino acid sequence that includes one or more alterations as described in TABLE 9 and the portion, other than the alteration described in TABLE 9, of the engineered ACL has at least 95% identical to SEQ ID NO: 2. In some embodiments, an engineered ACL provided herein has an amino acid sequence that includes one or more alterations as described in TABLE 9 and the portion, other than the alteration described in TABLE 9, of the engineered ACL has at least 98% identical to SEQ ID NO: 2. In some embodiments, an engineered ACL provided herein has an amino acid sequence that includes one or more alterations as described in TABLE 9 and the portion, other than the alteration described in TABLE 9, of the engineered ACL has at least 99% identical to SEQ ID NO: 2. In some embodiments, an engineered ACL provided herein has an amino acid sequence that includes one or more alterations as described in TABLE 9 and the portion, other than the alteration described in TABLE 9, of the engineered ACL is identical to SEQ ID NO: 2.

In some embodiments, an engineered ACL provided herein has an amino acid sequence that is a variant of SEQ ID NO: 2 that includes one or more alterations as described in TABLE 9 and/or one or more, or a combination of alterations, as described in TABLE 8, wherein the portion, other than the one or more alterations described in TABLE 9 or the one or more, or combination of alterations, as described in TABLE 8, of the engineered ACL has at least 65%, 70%, 75%, 80%, 85%, 90%, 95%, 98% or 99% sequence identity, or is identical, to an amino acid sequence referenced as SEQ ID NO: 2. Accordingly, in some embodiments, an engineered ACL provided herein has an amino acid sequence that includes one or more alterations as described in TABLE 9 and/or one or more, or a combination of alterations, as described in TABLE 8 and the portion, other than the alteration described in TABLE 9 or the one or more, or combination of alterations, as described in TABLE 8, of the engineered ACL has at least 65% sequence identity to SEQ ID NO: 2. In some embodiments, an engineered ACL provided herein has an amino acid sequence that includes one or more alterations as described in TABLE 9 and/or one or more, or a combination of alterations, as described in TABLE 8 and the portion, other than the alteration described in TABLE 9 or the one or more, or combination of alterations, as described in TABLE 8, of the engineered ACL has at least 70% sequence identity to SEQ ID NO: 2. In some embodiments, an engineered ACL provided herein has an amino acid sequence that includes one or more alterations as described in TABLE 9 and/or one or more, or a combination of alterations, as described in TABLE 8 and the portion, other than the alteration described in TABLE 9 or the one or more, or combination of alterations, as described in TABLE 8, of the engineered ACL has at least 75% sequence identity to SEQ ID NO: 2. In some embodiments, an engineered ACL provided herein has an amino acid sequence that includes one or more alterations as described in TABLE 9 and/or one or more, or a combination of alterations, as described in TABLE 8 and the portion, other than the alteration described in TABLE 9 or the one or more, or combination of alterations, as described in TABLE 8, of the engineered ACL has at least 80% sequence identity to SEQ ID NO: 2. In some embodiments, an engineered ACL provided herein has an amino acid sequence that includes one or more alterations as described in TABLE 9 and/or one or more, or a combination of alterations, as described in TABLE 8 and the portion, other than the alteration described in TABLE 9 or the one or more, or combination of alterations, as described in TABLE 8, of the engineered ACL has at least 85% sequence identity to SEQ ID NO: 2. In some embodiments, an engineered ACL provided herein has an amino acid sequence that includes one or more alterations as described in TABLE 9 and/or one or more, or a combination of alterations, as described in TABLE 8 and the portion, other than the alteration described in TABLE 9 or the one or more, or combination of alterations, as described in TABLE 8, of the engineered ACL has at least 90% sequence identity to SEQ ID NO: 2. In some embodiments, an engineered ACL provided herein has an amino acid sequence that includes one or more alterations as described in TABLE 9 and/or one or more, or a combination of alterations, as described in TABLE 8 and the portion, other than the alteration described in TABLE 9 or the one or more, or combination of alterations, as described in TABLE 8, of the engineered ACL has at least 95% sequence identity to SEQ ID NO: 2. In some embodiments, an engineered ACL provided herein has an amino acid sequence that includes one or more alterations as described in TABLE 9 and/or one or more, or a combination of alterations, as described in TABLE 8 and the portion, other than the alteration described in TABLE 9 or the one or more, or combination of alterations, as described in TABLE 8, of the engineered ACL has at least 98% sequence identity to SEQ ID NO: 2. In some embodiments, an engineered ACL provided herein has an amino acid sequence that includes one or more alterations as described in TABLE 9 and/or one or more, or a combination of alterations, as described in TABLE 8 and the portion, other than the alteration described in TABLE 9 or the one or more, or combination of alterations, as described in TABLE 8, of the engineered ACL has at least 99% sequence identity to SEQ ID NO: 2. In some embodiments, an engineered ACL provided herein has an amino acid sequence that includes one or more alterations as described in TABLE 9 and/or one or more, or a combination of alterations, as described in TABLE 8 and the portion, other than the alteration described in TABLE 9 or the one or more, or combination of alterations, as described in TABLE 8, of the engineered ACL is identical to SEQ ID NO: 2.

Sequence identity, homology or similarity refers to sequence similarity between two polypeptides or between two nucleic acid molecules. Identity can be determined by comparing a position in each sequence which may be aligned for purposes of comparison. When a position in the compared sequence is occupied by the same base or amino acid, then the molecules are identical at that position. A degree of homology between sequences is a function of the number of matching or homologous positions shared by the sequences. A polypeptide or polypeptide region (or a polynucleotide or polynucleotide region) has a certain percentage (for example, 65%, 70%, 75%, 80%, 85%, 90%, 95%, 98% or 99%) of “sequence identity” to another sequence means that, when aligned, that percentage of amino acids (or nucleotide bases) are the same in comparing the two sequences. The alignment of two sequences to determine their percent sequence identity can be done using software programs known in the art, such as, for example, those described in Ausubel et al., Current Protocols in Molecular Biology, John Wiley and Sons, Baltimore, MD (1999). Preferably, default parameters are used for the alignment. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases =non-redundant, GenBank+EMBL+DDBJ+PDB+GenBank CDS translations+SwissProtein+SPupdate+PIR. Details of these programs can be found at the National Center for Biotechnology Information (see also Altschul et al., “J. Mol. Biol. 215:403-410 (1990)).

Methods of generating and assaying the engineered ACLs described herein are well known to one of skill in the art. Examples of such methods are described in the Examples provided herein. Any of a variety of methods can be used to generate an engineered ACL disclosed herein. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). One non-limiting example of a method for preparing an engineered ACL is to express recombinant nucleic acids encoding the engineered ACL in a suitable microbial organism, such as a bacterial cell, a yeast cell, or other suitable cell, using methods well known in the art.

In some embodiments, an engineered ACL provided herein is an isolated ACL. An isolated engineered ACL provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

### Acetylating Aldehyde Dehydrogenase (ALDH)

In some embodiments, provided herein is an engineered ALDH that is a variant of a wild-type ALDH (e.g., SEQ ID NO: 3-7). Such an engineered ALDH includes one or more alterations at a position described in TABLE 12, and, in some embodiments, a combination of alterations described in TABLE 12, and has higher activity relative to the WT ALDH (e.g., SEQ ID NO: 3-7) as described herein. In certain embodiments, provided herein is an engineered ALDH that is a variant of a WT ALDH (e.g., SEQ ID NO: 3-7). Such an engineered ALDH includes one or more alterations at a position described in TABLE 12, and, in some embodiments, a combination of alterations described in TABLE 12, and has higher activity relative to the WT ALDH (e.g., SEQ ID NO: 3-7) as described herein. While the ALDH set forth in SEQ ID NO: 3 is occasionally used herein as a reference WT ALDH, it is understood that any WT ALDH disclosed herein (i.e., SEQ ID NOS: 4-7) may be utilized as the reference WT ALDH. Accordingly, reference to SEQ ID NO: 3 also refers to any one of SEQ ID NOS: 4-7. In some embodiments, the term “activity” as used herein to describe enzymatic activity, refers to catalytic activity, substrate specificity, substrate affinity, or any combination thereof.

In some embodiments, an engineered ALDH provided herein is capable of catalyzing the conversion of: 1) HHA-CoA to 6-hydroxyhexanal (6-HH); 2) adipyl-CoA to adipate semialdehyde, 3) acetyl-CoA to acetate, 4) succinyl-CoA to succinate, 5) 3-hydroxyadipyl-CoA to 4-HB, 6) 6ACA-CoA to 6ACA-SADH and 7) 3HB-CoA to 3-hydroxybutyrlaldehyde (3HB-ADH). Accordingly, in some embodiments, an engineered ALDH provided herein is capable of catalyzing the conversion of HHA-CoA to 6-hydroxyhexanal (6-HH). In some embodiments, an engineered ALDH provided herein is capable of catalyzing the conversion of adipyl-CoA to adipate semialdehyde. In some embodiments, an engineered ALDH provided herein is capable of catalyzing the conversion of acetyl-CoA to acetate. In some embodiments, an engineered ALDH provided herein is capable of catalyzing the conversion of succinyl-CoA to succinate. In some embodiments, an engineered ALDH provided herein is capable of catalyzing the conversion of 3-hydroxyadipyl-CoA to 4-HB. In some embodiments, an engineered ALDH provided herein is capable of catalyzing the conversion of 6ACA-CoA to 6ACA-SADH. In some embodiments, an engineered ALDH provided herein is capable of catalyzing the conversion of 3HB-CoA to 3-hydroxybutyrlaldehyde (3HB-ADH).

In some embodiments, an engineered ALDH as described herein has higher catalytic activity in the conversion of select substrates over other substrates. For example, in some embodiments, an engineered ALDH as described herein has: 1) higher specificity for conversion of HHA-CoA to 6-HH over conversion of adipyl-CoA to adipate semialdehyde, 2) higher specificity for conversion of HHA-CoA to 6-HH over conversion of acetyl-CoA to acetate, 3) higher specificity for conversion of HHA-CoA to 6-HH over conversion of succinyl-CoA to succinate, 4) higher specificity for conversion of HHA-CoA to 6-HH over conversion of 3-hydroxyadipyl-CoA to 4-HB, and 5) higher specificity for conversion of HHA-CoA to 6-HH over conversion of 3HB-CoA to 3HB-ADH, or any combination thereof. In another example, in some embodiments, an engineered ALDH as described herein has: 1) higher specificity for conversion of 6ACA-CoA to 6ACA-SADH over conversion of adipyl-CoA to adipate semialdehyde, 2) higher specificity for conversion of 6ACA-CoA to 6ACA-SADH over conversion of acetyl-CoA to acetate, 3) higher specificity for conversion of 6ACA-CoA to 6ACA-SADH over conversion of succinyl-CoA to succinate, 4) higher specificity for conversion of 6ACA-CoA to 6ACA-SADH over conversion of 3-hydroxyadipyl-CoA to 4-HB, and 5) higher specificity for conversion of 6ACA-CoA to 6ACA-SADH over conversion of 3HB-CoA to 3HB-ADH, or any combination thereof. Accordingly, in some embodiments, an engineered ALDH as described herein has: 1) higher specificity for conversion of HHA-CoA to 6-HH and/or 6ACA-CoA to 6ACA-SADH over conversion of adipyl-CoA to adipate semialdehyde, 2) higher specificity for conversion of HHA-CoA to 6-HH and/or 6ACA-CoA to 6ACA-SADH over conversion of acetyl-CoA to acetate, 3) higher specificity for conversion of HHA-CoA to 6-HH and/or 6ACA-CoA to 6ACA-SADH over conversion of succinyl-CoA to succinate, 4) higher specificity for conversion of HHA-CoA to 6-HH and/or 6ACA-CoA to 6ACA-SADH over conversion of 3-hydroxyadipyl-CoA to 4-HB, and 5) higher specificity for conversion of HHA-CoA to 6-HH and/or 6ACA-CoA to 6ACA-SADH over conversion of 3HB-CoA to 3HB-ADH, or any combination thereof.

In some embodiments, provided herein is an engineered ALDH comprising a variant of amino acid sequence SEQ ID NO: 3, wherein the engineered ALDH comprises one or more alterations at a position described in TABLE 12. In some embodiments, such an engineered ALDH is capable of catalyzing the conversion of: 1) HHA-CoA to 6-HH; 2) adipyl-CoA to adipate semialdehyde, 3) acetyl-CoA to acetate, 4) succinyl-CoA to succinate, 5) 3-hydroxyadipyl-CoA to 4-HB, 6) 6ACA-CoA to 6ACA-SADH and 7) 3HB-CoA to 3-3HB-ADH. Accordingly, in some embodiments, such an engineered ALDH comprising a variant of amino acid sequence SEQ ID NO: 3 is capable of catalyzing the conversion of HHA-CoA to 6-HH. In some embodiments, an engineered ALDH comprising a variant of amino acid sequence SEQ ID NO: 3 is capable of catalyzing the conversion of adipyl-CoA to adipate semialdehyde. In some embodiments, an engineered ALDH comprising a variant of amino acid sequence SEQ ID NO: 3 is capable of catalyzing the conversion of acetyl-CoA to acetate. In some embodiments, an engineered ALDH comprising a variant of amino acid sequence SEQ ID NO: 3 is capable of catalyzing the conversion of succinyl-CoA to succinate. In some embodiments, an engineered ALDH comprising a variant of amino acid sequence SEQ ID NO: 3 is capable of catalyzing the conversion of 3-hydroxyadipyl-CoA to 4-HB. In some embodiments, an engineered ALDH comprising a variant of amino acid sequence SEQ ID NO: 3 is capable of catalyzing the conversion of 6ACA-CoA to 6ACA-SADH. In some embodiments, an engineered ALDH comprising a variant of amino acid sequence SEQ ID NO: 3 is capable of catalyzing the conversion of 3HB-CoA to 3HB-ADH.

It is understood that the engineered ALDHs as described herein can carry out a similar enzymatic reaction as the wild-type ALDHs (e.g., SEQ ID NO: 3-7) as discussed above. It is further understood that the variants of the ALDH enzyme can include alterations that provide a beneficial characteristic to the engineered ALDH, including but not limited to, increased activity (e.g., ability to catalyze a reaction described herein and/or selectivity for a substrate, such as HHA-CoA or 6ACA-CoA) as described herein (see, e.g., Example 6). In some embodiments, the engineered ALDH can exhibit an activity that is at least the same or higher than the wild-type ALDH (e.g., SEQ ID NO: 3-7), that is, it has activity that is the same or higher than an ALDH without the variant at the same amino acid position(s). In some embodiments, the engineered ALDH can exhibit two or more activities (e.g., ability to catalyze a reaction described herein and selectivity for a substrate, such as HHA-CoA or 6ACA-CoA) that are at least the same or higher than the wild-type ALDH (e.g., SEQ ID NO: 3-7), that is, it has two or more activities that are the same or higher than an ALDH without the variant at the same amino acid position(s). For example, the engineered ALDHs provided here can have one or more activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher over a wild-type ALDH (see, e.g., Example 6). In some embodiments, an engineered ALDH provided herein has an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an ALDH consisting of the amino acid sequence of SEQ ID NO: 3. In some embodiments, an engineered ALDH provided herein has an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an ALDH consisting of the amino acid sequence of SEQ ID NO: 3. In some embodiments, an engineered ALDH provided herein has an activity that is at least 10% higher. In some embodiments, an engineered ALDH provided herein has an activity that is at least 20% higher. In some embodiments, an engineered ALDH provided herein has an activity that is at least 30% higher. In some embodiments, an engineered ALDH provided herein has an activity that is at least 40% higher. In some embodiments, an engineered ALDH provided herein has an activity that is at least 50% higher. In some embodiments, an engineered ALDH provided herein has an activity that is at least 60% higher. In some embodiments, an engineered ALDH provided herein has an activity that is at least 70% higher. In some embodiments, an engineered ALDH provided herein has an activity that is at least 80% higher. In some embodiments, an engineered ALDH provided herein has an activity that is at least 90% higher. In some embodiments, an engineered ALDH provided herein has an activity that is at least 100% higher. In some embodiments, an engineered ALDH provided herein has an activity that is at least 110% higher. In some embodiments, an engineered ALDH provided herein has an activity that is at least 120% higher. In some embodiments, an engineered ALDH provided herein has an activity that is at least 130% higher. In some embodiments, an engineered ALDH provided herein has an activity that is at least 140% higher. In some embodiments, an engineered ALDH provided herein has an activity that is at least 150% higher. In some embodiments, an engineered ALDH provided herein has an activity that is at least 160% higher. In some embodiments, an engineered ALDH provided herein has an activity that is at least 170% higher. In some embodiments, an engineered ALDH provided herein has an activity that is at least 180% higher. In some embodiments, an engineered ALDH provided herein has an activity that is at least 190% higher. In some embodiments, an engineered ALDH provided herein has an activity that is at least 200% higher. It is understood that activity refers to the ability of an engineered ALDH described herein to convert a substrate to a product relative to a wild-type ALDH (SEQ ID NO: 3) under the same assay conditions, such as those described herein (see, e.g., Example 6).

In some embodiments, the activity of an ALDH described herein is measured as the catalytic constant (k_{cat}) value or turnover number. In some embodiments, the k_{cat} is at least 0.1 s⁻¹, at least 0.2 s⁻¹, at least 0.3 s⁻¹, at least 0.4 s⁻¹, at least 0.5 s⁻¹, at least 0.6 s⁻¹, at least 0.7 s⁻¹, at least 0.8 s⁻¹, at least 0.9 s⁻¹, at least 1 s⁻¹, at least 2 s⁻¹, at least 3 s⁻¹, at least 4 s⁻¹, at least 5 s⁻¹, at least 6 s⁻¹, at least 7 s⁻¹, at least 8 s⁻¹, at least 9 s⁻¹, at least 10 s⁻¹, at least 11 s⁻¹, at least 12 s⁻¹, at least 13 s⁻¹, at least 14 s⁻¹, at least 15 s⁻¹, at least 16 s⁻¹, at least 17 s⁻¹, at least 18 s⁻¹, at least 19 s⁻¹, at least 20 s⁻¹, at least 21 s⁻¹, at least 22 s⁻¹, at least 23 s⁻¹, at least 24 s⁻¹, at least 25 s⁻¹, at least 26 s⁻¹, at least 27 s⁻¹, at least 28 s⁻¹, at least 29 s⁻¹, at least 30 s⁻¹, at least 31 s⁻¹, at least 32 s⁻¹, at least 33 s⁻¹, at least 34 s⁻¹, at least 35 s⁻¹, at least 36 s⁻¹, at least 37 s⁻¹, at least 38 s⁻¹, at least 39 s⁻¹, at least 40 s⁻¹, at least 41 s⁻¹, at least 42 s⁻¹, at least 43 s⁻¹, at least 44 s⁻¹, at least 45 s⁻¹, at least 46 s⁻¹, at least 47 s⁻¹, at least 48 s⁻¹, at least 49 s⁻¹, at least 50 s⁻¹, at least 51 s⁻¹, at least 52 s⁻¹, at least 53 s⁻¹, at least 54 s⁻¹, at least 55 s⁻¹, at least 56 s⁻¹, at least 57 s⁻¹, at least 58 s⁻¹, at least 59 s⁻¹, at least 60 s⁻¹, at least 61 s⁻¹, at least 62 s⁻¹, at least 63 s⁻¹, at least 64 s⁻¹, at least 65 s⁻¹, at least 66 s⁻¹, at least 67 s⁻¹, at least 68 s⁻¹, at least 69 s⁻¹, at least 70 s⁻¹, at least 71 s⁻¹, at least 72 s⁻¹, at least 73 s⁻¹, at least 74 s⁻¹, at least 75 s⁻¹, at least 76 s⁻¹, at least 77 s⁻¹, at least 78 s⁻¹, at least 79 s⁻¹, at least 80 s⁻¹, at least 81 s⁻¹, at least 82 s⁻¹, at least 83 s⁻¹, at least 84 s⁻¹, at least 85 s⁻¹, at least 86 s⁻¹, at least 87 s⁻¹, at least 88 s⁻¹, at least 89 s⁻¹, at least 90 s⁻¹, at least 91 s⁻¹, at least 92 s⁻¹, at least 93 s⁻¹, at least 94 s⁻¹, at least 95 s⁻¹, at least 96 s⁻¹, at least 97 s⁻¹, at least 98 s⁻¹, at least 99 s⁻¹, at least 100 s⁻¹, at least 500 s⁻¹, or at least 1000 s⁻¹. In some embodiments, the K_{cat} is between 1 s⁻¹ and 100 s⁻¹, between 5 s⁻¹ and 50 s⁻¹, or between 10 s⁻¹ and 50 s⁻¹.

In some embodiments, the activity of an ALDH described herein is measured as the Michaelis constant (Kₘ). In some embodiments, the Kₘ is less than 0.1 μM, less than 0.2 μM, less than 0.3 μM, less than 0.4 μM, less than 0.5 μM, less than 0.6 μM, less than 0.7 μM, less than 0.8 μM, less than 0.9 μM, less than 1 μM, less than 2 μM, less than 3 μM, less than 4 μM, less than 5 μM, less than 6 μM, less than 7 μM, less than 8 μM, less than 9 μM, less than 10 μM, less than 11 μM, less than 12 μM, less than 13 μM, less than 14 μM, less than 15 μM, less than 16 μM, less than 17 μM, less than 18 μM, less than 19 μM, less than 20 μM, less than 21 μM, less than 22 μM, less than 23 μM, less than 24 μM, less than 25 μM, less than 26 μM, less than 27 μM, less than 28 μM, less than 29 μM, less than 30 μM, less than 31 μM, less than 32 μM, less than 33 μM, less than 34 μM, less than 35 μM, less than 36 μM, less than 37 μM, less than 38 μM, less than 39 μM, less than 40 μM, less than 41 μM, less than 42 μM, less than 43 μM, less than 44 μM, less than 45 μM, less than 46 μM, less than 47 μM, less than 48 μM, less than 49 μM, less than 50 μM, less than 51 μM, less than 52 μM, less than 53 μM, less than 54 μM, less than 55 μM, less than 56 μM, less than 57 μM, less than 58 μM, less than 59 μM, less than 60 μM, less than 61 μM, less than 62 μM, less than 63 μM, less than 64 μM, less than 65 μM, less than 66 μM, less than 67 μM, less than 68 μM, less than 69 μM, less than 70 μM, less than 71 μM, less than 72 μM, less than 73 μM, less than 74 μM, less than 75 μM, less than 76 μM, less than 77 μM, less than 78 μM, less than 79 μM, less than 80 μM, less than 81 μM, less than 82 μM, less than 83 μM, less than 84 μM, less than 85 μM, less than 86 μM, less than 87 μM, less than 88 μM, less than 89 μM, less than 90 μM, less than 91 μM, less than 92 μM, less than 93 μM, less than 94 μM, less than 95 μM, less than 96 μM, less than 97 μM, less than 98 μM, less than 99 μM, less than 100 μM, less than 200 μM, less than 300 μM, less than 400 μM, less than 500 μM, less than 600 μM, less than 700 μM, less than 800 μM, less than 900 μM, less than 1000 μM, less than 1100 μM, less than 1200 μM, less than 1300 μM, less than 1400 μM, less than 1500 μM, less than 1600 μM, less than 1700 μM, less than 1800 μM, less than 1900 μM, or less than 2000 μM. In some embodiments, the Kₘ is between 0.1 μM and 2000 μM, between 1 μM and 1000 μM, between 1 μM and 100 μM, between 0.1 μM and 1000 pM, between 100 μM and 2000 μM, between 100 μM and 1000 μM, between 1000 μM and 2000 μM, between 500 μM and 1500 μM, between 500 μM and 1500 μM, or between 0.1 μM and 1000 μM.

In some embodiments, the activity of an ALDH described herein is measured as the catalytic efficiency (k_{cat}/kₘ). In some embodiments, the catalytic efficiency is measured in units of s⁻¹ mM⁻¹. In some embodiments, the catalytic efficiency is greater than 0.1, greater than 0.2, greater than 0.3, greater than 0.4, greater than 0.5, greater than 0.6, greater than 0.7, greater than 0.8, greater than 0.9, greater than 1, greater than 2, greater than 3, greater than 4, greater than 5, greater than 6, greater than 7, greater than 8, greater than 9, greater than 10, greater than 11, greater than 12, greater than 13, greater than 14, greater than 15, greater than 16, greater than 17, greater than 18, greater than 19, greater than 20, greater than 21, greater than 22, greater than 23, greater than 24, greater than 25, greater than 26, greater than 27, greater than 28, greater than 29, greater than 30, greater than 31, greater than 32, greater than 33, greater than 34, greater than 35, greater than 36, greater than 37, greater than 38, greater than 39, greater than 40, greater than 41, greater than 42, greater than 43, greater than 44, greater than 45, greater than 46, greater than 47, greater than 48, greater than 49, greater than 50, greater than 51, greater than 52, greater than 53, greater than 54, greater than 55, greater than 56, greater than 57, greater than 58, greater than 59, greater than 60, greater than 61, greater than 62, greater than 63, greater than 64, greater than 65, greater than 66, greater than 67, greater than 68, greater than 69, greater than 70, greater than 71, greater than 72, greater than 73, greater than 74, greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, greater than 99, greater than 100, greater than 500, greater than 1000 s⁻¹ mM⁻¹. In some embodiments, the catalytic efficiency (k_{cat}/kₘ) is between 1 and 30 s⁻¹ mM⁻¹, between 5 and 30 s⁻¹ mM⁻¹, between 1 and 10 s⁻¹ mM⁻¹, between 10 and 30 s⁻¹ mM⁻¹, or between 20 and 30 s⁻¹ mM⁻¹.

In some embodiments, an engineered ALDH provided herein is a variant of a reference polypeptide, wherein the reference polypeptide has an amino acid sequence of SEQ ID NO: 3, and the engineered ALDH has one or more alterations at a position described in TABLE 12 relative to SEQ ID NO: 3. Accordingly, in some embodiments, an engineered ALDH provided herein includes one or more amino acid alterations at a residue corresponding to position 2, 3, 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, 36, 37, 38, 39, 40, 41, 42, 43, 44, 45, 47, 48, 49, 50, 51, 52, 53, 54, 55, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 104, 106, 107, 108, 109, 110, 111, 112, 113, 114, 115, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, 201, 202, 203, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 243, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 288, 289, 290, 291, 292, 293, 294, 295, 296, 297, 298, 299, 300, 301, 302, 303, 304, 305, 306, 307, 308, 310, 311, 312, 313, 314, 315, 316, 317, 318, 319, 320, 321, 322, 323, 324, 325, 326, 327, 328, 329, 330, 331, 332, 333, 334, 335, 337, 338, 339, 341, 342, 343, 344, 345, 346, 347, 348, 349, 350, 351, 352, 354, 355, 356, 357, 358, 359, 360, 361, 362, 363, 364, 366, 368, 369, 370, 371, 372, 373, 374, 375, 376, 377, 378, 379, 380, 381, 382, 383, 384, 385, 386, 387, 388, 389, 390, 391, 392, 393, 394, 396, 397, 398, 399, 400, 401, 402, 403, 405, 406, 407, 408, 410, 411, 412, 413, 414, 415, 416, 418, 420, 421, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, more than one of the foregoing, or a combination thereof, in SEQ ID NO: 3. In some embodiments, an engineered ALDH provided herein includes one or more amino acid alterations at a residue corresponding to position 2, 3, 4, 5, 6, 7, 9, 11, 12, 15, 16, 17, 18, 20, 21, 22, 23, 24, 25, 26, 27, 28, 30, 31, 32, 33, 34, 35, 36, 37, 38, 39, 40, 41, 42, 43, 44, 45, 47, 48, 49, 51, 52, 54, 55, 58, 61, 62, 63, 64, 65, 66, 67, 70, 71, 73, 74, 75, 77, 78, 79, 80, 81, 82, 83, 84, 86, 88, 89, 90, 91, 92, 93, 94, 95, 96, 97, 98, 99, 100, 101, 102, 104, 107, 111, 117, 119, 120, 121, 122, 124, 125, 126, 128, 129, 130, 133, 136, 144, 145, 146, 147, 148, 149, 150, 151, 152, 153, 154, 155, 156, 157, 158, 159, 160, 162, 164, 165, 166, 167, 168, 169, 170, 171, 172, 174, 175, 176, 177, 180, 181, 183, 184, 187, 189, 190, 194, 196, 197, 198, 199, 200, 201, 202, 203, 206, 207, 208, 212, 214, 215, 216, 217, 218, 219, 221, 222, 223, 224, 225, 227, 228, 229, 230, 231, 232, 233, 234, 237, 238, 240, 243, 245, 246, 248, 249, 250, 251, 252, 253, 254, 255, 256, 257, 258, 259, 260, 261, 262, 263, 264, 265, 266, 267, 270, 271, 272, 273, 274, 275, 276, 277, 278, 279, 280, 281, 282, 283, 284, 285, 286, 288, 290, 291, 293, 294, 295, 296, 299, 300, 301, 302, 303, 304, 305, 306, 307, 308, 310, 311, 312, 313, 315, 316, 317, 318, 319, 320, 321, 322, 323, 324, 325, 326, 327, 328, 329, 330, 331, 332, 333, 334, 335, 339, 341, 342, 343, 344, 345, 346, 347, 348, 349, 350, 351, 354, 355, 356, 358, 359, 360, 362, 363, 366, 370, 371, 372, 373, 374, 375, 376, 377, 378, 379, 380, 381, 382, 383, 384, 385, 386, 387, 388, 389, 390, 392, 393, 394, 396, 397, 400, 401, 402, 405, 406, 407, 410, 411, 414, 420, 421, 423, 425, 426, 427, 428, 431, 433, 435, 436, 437, 438, 439, 440, 441, 443, 444, 446, 447, 448, 449, 450, 452, 453, 454, 455, 457, 458, 459, more than one of the foregoing, or a combination thereof, in SEQ ID NO: 3. In some embodiments, an engineered ALDH provided herein includes one or more amino acid alterations at a residue corresponding to position 42, 119, 149, 158, 174, 246, 259, 263, 299, 327, 348, 370, 392, 401, 427, more than one of the foregoing, or a combination thereof, in SEQ ID NO: 3.

In some embodiments, an engineered ALDH provided herein includes one or more alterations at a position described in TABLE 12, wherein the one or more amino acid alterations are conservative amino acid substitutions. In some embodiments, an engineered ALDH provided herein includes one or more conservative amino acid substitutions relative to an alteration described in TABLE 12. As a non-limiting example, a conservative amino acid substitution relative to the M121L substitution in SEQ ID NO: 3 may include substitution of M121 for another non-polar (hydrophobic) amino acid (e.g., Cys (C), Ala (A), Val (V), Ile (I), Pro (P), Phe (F), Met (M), Trp (W), Gly (G), or Tyr (Y)). In some embodiments, an engineered ALDH provided herein includes one or more alterations at a position described in TABLE 12, wherein the one or more amino acid alterations are non-conservative amino acid substitutions. In some embodiments, an engineered ALDH provided herein includes a conservative amino acid substitution and/or non-conservative amino acid substitution in 1 to 100 amino acid positions as set forth in TABLE 12.

In some embodiments, an engineered ALDH provided herein can further include a conservative amino acid substitution in from 1 to 50 amino acid positions, or alternatively from 2 to 50 amino acid positions, or alternatively from 3 to 50 amino acid positions, or alternatively from 4 to 50 amino acid positions, or alternatively from 5 to 50 amino acid positions, or alternatively from 6 to 50 amino acid positions, or alternatively from 7 to 50 amino acid positions, or alternatively from 8 to 50 amino acid positions, or alternatively from 9 to 50 amino acid positions, or alternatively from 10 to 50 amino acid positions, or alternatively from 15 to 50 amino acid positions, or alternatively from 20 to 50 amino acid positions, or alternatively from 30 to 50 amino acid positions, or alternatively from 40 to 50 amino acid positions, or alternatively from 45 to 50 amino acid positions, or any integer therein, wherein the positions are other than the variant amino acid positions set forth in TABLE 12. In some aspects, such a conservative amino acid sequence is a chemically conservative or an evolutionary conservative amino acid substitution. Methods of identifying conservative amino acids are well known to one of skill in the art, any one of which can be used to generate the isolated engineered ALDHs described herein.

An engineered ALDH provided herein may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 alterations relative to a wild-type ALDH (SEQ ID NO: 3). An engineered ALDH provided herein may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, at most 101, at most 102, at most 103, at most 104, at most 105, at most 106, at most 107, at most 108, at most 109, at most 110, at most 111, at most 112, at most 113, at most 114, at most 115, at most 116, at most 117, at most 118, at most 119, at most 120, at most 121, at most 122, at most 123, at most 124, at most 125, at most 126, at most 127, at most 128, at most 129, at most 130, at most 131, at most 132, at most 133, at most 134, at most 135, at most 136, at most 137, at most 138, at most 139, at most 140, at most 141, at most 142, at most 143, at most 144, at most 145, at most 146, at most 147, at most 148, at most 149, at most 150, at most 151, at most 152, at most 153, at most 154, at most 155, at most 156, at most 157, at most 158, at most 159, at most 160, at most 161, at most 162, at most 163, at most 164, at most 165, at most 166, at most 167, at most 168, at most 169, at most 170, at most 171, at most 172, at most 173, at most 174, at most 175, at most 176, at most 177, at most 178, at most 179, at most 180, at most 181, at most 182, at most 183, at most 184, at most 185, at most 186, at most 187, at most 188, at most 189, at most 190, at most 191, at most 192, at most 193, at most 194, at most 195, at most 196, at most 197, at most 198, at most 199, at most 200, at most 201, at most 202, at most 203, at most 204, at most 205, at most 206, at most 207, at most 208, at most 209, at most 210, at most 211, at most 212, at most 213, at most 214, at most 215, at most 216, at most 217, at most 218, at most 219, at most 220, at most 221, at most 222, at most 223, at most 224, at most 225, at most 226, at most 227, at most 228, at most 229, at most 230, at most 231, at most 232, at most 233, at most 234, at most 235, at most 236, at most 237, at most 238, at most 239, at most 240, at most 241, at most 242, at most 243, at most 244, at most 245, at most 246, at most 247, at most 248, at most 249, or at most 250 alterations relative to a wild-type ALDH (SEQ ID NO: 3). The one or more alterations may be located at one or more positions corresponding to the one or more positions described in TABLE 12. The one or more alterations may be located at one or more positions corresponding to one or more positions in SEQ ID NO: 3. As used herein, the phrase “a residue corresponding to position X in SEQ ID NO: Y” refers to a residue at a corresponding position following an alignment of two sequences. For example, the residue in SEQ ID NO: 1 corresponding to position 370 in SEQ ID NO: 3 is the residue at position 370 in SEQ ID NO: 7. In some embodiments, a reference sequence is an ALDH that is not SEQ ID NO: 3. In some embodiments, a reference sequence is an ALDH in any one of SEQ ID NOS: 4-7. Accordingly, the one or more alterations may be located at one or more positions corresponding to one or more positions in SEQ ID NOS: 4-7.

An engineered ALDH provided herein can include any combination of the alterations set forth in TABLE 12. One alteration alone, or in combination, can produce an engineered ALDH that retains or improves the activity as described herein relative to a reference polypeptide, for example, the wild-type ALDH (SEQ ID NO: 3). In some embodiments, an engineered ALDH provided herein includes at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 alterations as set forth in TABLE 12, including up to an alteration at all of the positions identified in TABLE 12. In some embodiments, an engineered ALDH provided herein includes at least 2 alterations as set forth in TABLE 12. In some embodiments, an engineered ALDH provided herein includes at least 3 alterations as set forth in TABLE 12. In some embodiments, an engineered ALDH provided herein includes at least 4 alterations as set forth in TABLE 12. In some embodiments, an engineered ALDH provided herein includes at least 5 alterations as set forth in TABLE 12. In some embodiments, an engineered ALDH provided herein includes at least 6 alterations as set forth in TABLE 12. In some embodiments, an engineered ALDH provided herein includes at least 7 alterations as set forth in TABLE 12. In some embodiments, an engineered ALDH provided herein includes at least 8 alterations as set forth in TABLE 12. In some embodiments, an engineered ALDH provided herein includes at least 9 alterations as set forth in TABLE 12. In some embodiments, an engineered ALDH provided herein includes at least 10 alterations as set forth in TABLE 12. In some embodiments, an engineered ALDH provided herein includes at least 11 alterations as set forth in TABLE 12. In some embodiments, an engineered ALDH provided herein includes at least 12 alterations as set forth in TABLE 12. In some embodiments, an engineered ALDH provided herein includes at least 13 alterations as set forth in TABLE 12. In some embodiments, an engineered ALDH provided herein includes at least 14 alterations as set forth in TABLE 12. In some embodiments, an engineered ALDH provided herein includes at least 15 alterations as set forth in TABLE 12. In some embodiments, an engineered ALDH provided herein includes at least 16 alterations as set forth in TABLE 12. In some embodiments, an engineered ALDH provided herein includes at least 17 alterations as set forth in TABLE 12. In some embodiments, an engineered ALDH provided herein includes at least 18 alterations as set forth in TABLE 12. In some embodiments, an engineered ALDH provided herein includes at least 19 alterations as set forth in TABLE 12. In some embodiments, an engineered ALDH provided herein includes at least 20 alterations as set forth in TABLE 12. In some embodiments, an engineered ALDH provided herein includes at least 21 alterations as set forth in TABLE 12. In some embodiments, an engineered ALDH provided herein includes at least 22 alterations as set forth in TABLE 12. In some embodiments, an engineered ALDH provided herein includes at least 23 alterations as set forth in TABLE 12. In some embodiments, an engineered ALDH provided herein includes at least 10 alterations as set forth in TABLE 12. In some embodiments, an engineered ALDH provided herein includes at least 24 alterations as set forth in TABLE 12. In some embodiments, an engineered ALDH provided herein includes at least 25 alterations as set forth in TABLE 12. In some embodiments, an engineered ALDH provided herein includes at least 26 alterations as set forth in TABLE 12. In some embodiments, an engineered ALDH provided herein includes at least 27 alterations as set forth in TABLE 12. In some embodiments, an engineered ALDH provided herein includes at least 28 alterations as set forth in TABLE 12. In some embodiments, an engineered ALDH provided herein includes at least 29 alterations as set forth in TABLE 12. In some embodiments, an engineered ALDH provided herein includes at least 30 alterations as set forth in TABLE 12.

In certain embodiments, the one or more amino acid alterations of the engineered ALDH is an alteration described in TABLE 12. For example, in some embodiments, the one or more amino acid alterations result in an engineered ALDH having: P, Q, T at a residue corresponding to position 2 in SEQ ID NO: 3; E, K, M, R, S at a residue corresponding to position 3 in SEQ ID NO: 3; G, S at a residue corresponding to position 4 in SEQ ID NO: 3; I, T at a residue corresponding to position 5 in SEQ ID NO: 3; E, F, H, P, Q, R at a residue corresponding to position 6 in SEQ ID NO: 3; E, Q at a residue corresponding to position 7 in SEQ ID NO: 3; D at a residue corresponding to position 8 in SEQ ID NO: 3; I, V at a residue corresponding to position 9 in SEQ ID NO: 3; N at a residue corresponding to position 10 in SEQ ID NO: 3; A, D, E, Q, R at a residue corresponding to position 11 in SEQ ID NO: 3; A, I, L at a residue corresponding to position 12 in SEQ ID NO: 3; L at a residue corresponding to position 13 in SEQ ID NO: 3; D, E, K, Q at a residue corresponding to position 14 in SEQ ID NO: 3; K, R at a residue corresponding to position 15 in SEQ ID NO: 3; A at a residue corresponding to position 16 in SEQ ID NO: 3; H, K, Q at a residue corresponding to position 17 in SEQ ID NO: 3; E, K, Q, T at a residue corresponding to position 18 in SEQ ID NO: 3; F, H, M at a residue corresponding to position 20 in SEQ ID NO: 3; A, E, K, P, R at a residue corresponding to position 21 in SEQ ID NO: 3; A, K, Q at a residue corresponding to position 22 in SEQ ID NO: 3; L, W, Y at a residue corresponding to position 23 in SEQ ID NO: 3; E, K, R, S at a residue corresponding to position 24 in SEQ ID NO: 3; A, E, K, N, S at a residue corresponding to position 25 in SEQ ID NO: 3; F, L, M, T, W at a residue corresponding to position 26 in SEQ ID NO: 3; D, S, T at a residue corresponding to position 27 in SEQ ID NO: 3; W at a residue corresponding to position 28 in SEQ ID NO: 3; K, Q at a residue corresponding to position 29 in SEQ ID NO: 3; D, E, K, Q, R at a residue corresponding to position 30 in SEQ ID NO: 3; I, L, R at a residue corresponding to position 31 in SEQ ID NO: 3; A, E, N at a residue corresponding to position 32 in SEQ ID NO: 3; A, E, Q, R at a residue corresponding to position 33 in SEQ ID NO: 3; I, L, M, V at a residue corresponding to position 34 in SEQ ID NO: 3; C, I, L, M at a residue corresponding to position 35 in SEQ ID NO: 3; E, H, K at a residue corresponding to position 36 in SEQ ID NO: 3; A, K, R at a residue corresponding to position 37 in SEQ ID NO: 3; F, I, L, M, W at a residue corresponding to position 38 in SEQ ID NO: 3; A, R at a residue corresponding to position 39 in SEQ ID NO: 3; D, E, Q, R, W at a residue corresponding to position 40 in SEQ ID NO: 3; L, V at a residue corresponding to position 41 in SEQ ID NO: 3; C, L, M, V at a residue corresponding to position 42 in SEQ ID NO: 3; E, L, R at a residue corresponding to position 43 in SEQ ID NO: 3; E, K, Q, R at a residue corresponding to position 44 in SEQ ID NO: 3; H, Q, R at a residue corresponding to position 45 in SEQ ID NO: 3; D at a residue corresponding to position 47 in SEQ ID NO: 3; D, E, M, R at a residue corresponding to position 48 in SEQ ID NO: 3; F, I, M, W, Y at a residue corresponding to position 49 in SEQ ID NO: 3; G at a residue corresponding to position 50 in SEQ ID NO: 3; A, E, K, N, Q, R at a residue corresponding to position 51 in SEQ ID NO: 3; L at a residue corresponding to position 52 in SEQ ID NO: 3; E, I, S at a residue corresponding to position 53 in SEQ ID NO: 3; I, L, M, T at a residue corresponding to position 54 in SEQ ID NO: 3; E, Q, R at a residue corresponding to position 55 in SEQ ID NO: 3; E, G, Q, R at a residue corresponding to position 58 in SEQ ID NO: 3; F, I, L, Y at a residue corresponding to position 59 in SEQ ID NO: 3; A at a residue corresponding to position 60 in SEQ ID NO: 3; D, E, K, R at a residue corresponding to position 61 in SEQ ID NO: 3; A, F, I, M, V, W at a residue corresponding to position 62 in SEQ ID NO: 3; D, E, Q at a residue corresponding to position 63 in SEQ ID NO: 3; E, H at a residue corresponding to position 64 in SEQ ID NO: 3; E, R at a residue corresponding to position 65 in SEQ ID NO: 3; L, V at a residue corresponding to position 66 in SEQ ID NO: 3; E at a residue corresponding to position 67 in SEQ ID NO: 3; D, E at a residue corresponding to position 68 in SEQ ID NO: 3; H, I, L, M, N at a residue corresponding to position 69 in SEQ ID NO: 3; I, L, M, R at a residue corresponding to position 70 in SEQ ID NO: 3; D, E, F, H, Y at a residue corresponding to position 71 in SEQ ID NO: 3; A, R at a residue corresponding to position 72 in SEQ ID NO: 3; A, T at a residue corresponding to position 73 in SEQ ID NO: 3; D, E, R at a residue corresponding to position 74 in SEQ ID NO: 3; G, P, Y at a residue corresponding to position 75 in SEQ ID NO: 3; F, I, L, M, Y at a residue corresponding to position 76 in SEQ ID NO: 3; F, Y at a residue corresponding to position 77 in SEQ ID NO: 3; E, F, H, N, R, Y at a residue corresponding to position 78 in SEQ ID NO: 3; D, S, W, Y at a residue corresponding to position 79 in SEQ ID NO: 3; F, I, L, V at a residue corresponding to position 80 in SEQ ID NO: 3; A, E, R at a residue corresponding to position 81 in SEQ ID NO: 3; E, G, P at a residue corresponding to position 82 in SEQ ID NO: 3; E, Q at a residue corresponding to position 83 in SEQ ID NO: 3; Q, R at a residue corresponding to position 84 in SEQ ID NO: 3; A, T at a residue corresponding to position 85 in SEQ ID NO: 3; E, K, V at a residue corresponding to position 86 in SEQ ID NO: 3; T at a residue corresponding to position 87 in SEQ ID NO: 3; L, M, V at a residue corresponding to position 88 in SEQ ID NO: 3; L, P, V at a residue corresponding to position 89 in SEQ ID NO: 3; D, E, H, P, R, S at a residue corresponding to position 90 in SEQ ID NO: 3; D, E, Q at a residue corresponding to position 91 in SEQ ID NO: 3; D, M at a residue corresponding to position 92 in SEQ ID NO: 3; D, E at a residue corresponding to position 93 in SEQ ID NO: 3; A, D, G, N at a residue corresponding to position 94 in SEQ ID NO: 3; K, M, N, R at a residue corresponding to position 95 in SEQ ID NO: 3; H, K, N, R at a residue corresponding to position 96 in SEQ ID NO: 3; I, M, V at a residue corresponding to position 97 in SEQ ID NO: 3; I, M, T, Y at a residue corresponding to position 98 in SEQ ID NO: 3; K, Q, R, T at a residue corresponding to position 99 in SEQ ID NO: 3; Y, L, M, I at a residue corresponding to position 100 in SEQ ID NO: 3; K, Y, R, H at a residue corresponding to position 101 in SEQ ID NO: 3; E, Q, R, V at a residue corresponding to position 102 in SEQ ID NO: 3; V, Y, F, L, M at a residue corresponding to position 104 in SEQ ID NO: 3; I at a residue corresponding to position 106 in SEQ ID NO: 3; C, V at a residue corresponding to position 107 in SEQ ID NO: 3; A, C at a residue corresponding to position 108 in SEQ ID NO: 3; I, A, M at a residue corresponding to position 109 in SEQ ID NO: 3; M, L, I at a residue corresponding to position 110 in SEQ ID NO: 3; T, V, Y, I, S at a residue corresponding to position 111 in SEQ ID NO: 3; E at a residue corresponding to position 112 in SEQ ID NO: 3; F, S, W, Y at a residue corresponding to position 113 in SEQ ID NO: 3; N at a residue corresponding to position 114 in SEQ ID NO: 3; W, R, H, Y at a residue corresponding to position 115 in SEQ ID NO: 3; N, L, T, I at a residue corresponding to position 117 in SEQ ID NO: 3; N, A, L, M, S, F, V at a residue corresponding to position 118 in SEQ ID NO: 3; Q, I, L, M, T, S at a residue corresponding to position 119 in SEQ ID NO: 3; T, A, P, M at a residue corresponding to position 120 in SEQ ID NO: 3; V, A, L, C, I at a residue corresponding to position 121 in SEQ ID NO: 3; C, F, R, Y, H, N, S, G, V, W at a residue corresponding to position 122 in SEQ ID NO: 3; K, P at a residue corresponding to position 123 in SEQ ID NO: 3; V, L, M, I at a residue corresponding to position 124 in SEQ ID NO: 3; I, L, A, V at a residue corresponding to position 125 in SEQ ID NO: 3; A, P at a residue corresponding to position 126 in SEQ ID NO: 3; M at a residue corresponding to position 127 in SEQ ID NO: 3; I at a residue corresponding to position 128 in SEQ ID NO: 3; A, M at a residue corresponding to position 129 in SEQ ID NO: 3; T, S, A at a residue corresponding to position 130 in SEQ ID NO: 3; R at a residue corresponding to position 131 in SEQ ID NO: 3; C at a residue corresponding to position 132 in SEQ ID NO: 3; T, C at a residue corresponding to position 133 in SEQ ID NO: 3; I, C, V at a residue corresponding to position 134 in SEQ ID NO: 3; V, M at a residue corresponding to position 135 in SEQ ID NO: 3; L, M, F, I at a residue corresponding to position 136 in SEQ ID NO: 3; R, K at a residue corresponding to position 137 in SEQ ID NO: 3; G at a residue corresponding to position 138 in SEQ ID NO: 3; S, G at a residue corresponding to position 139 in SEQ ID NO: 3; E at a residue corresponding to position 140 in SEQ ID NO: 3; Q, K, Y, E at a residue corresponding to position 141 in SEQ ID NO: 3; T, S at a residue corresponding to position 142 in SEQ ID NO: 3; P at a residue corresponding to position 143 in SEQ ID NO: 3; L, N, R at a residue corresponding to position 144 in SEQ ID NO: 3; T, C, V at a residue corresponding to position 145 in SEQ ID NO: 3; T, C, A, N at a residue corresponding to position 146 in SEQ ID NO: 3; I, M, A at a residue corresponding to position 147 in SEQ ID NO: 3; Y, L, A, E, H, R at a residue corresponding to position 148 in SEQ ID NO: 3; V, I, L, M, F at a residue corresponding to position 149 in SEQ ID NO: 3; I, M, G, A at a residue corresponding to position 150 in SEQ ID NO: 3; D, K, R, Q at a residue corresponding to position 151 in SEQ ID NO: 3; L, M, I at a residue corresponding to position 152 in SEQ ID NO: 3; F, V, C, M at a residue corresponding to position 153 in SEQ ID NO: 3; H, A, Q, K, R at a residue corresponding to position 154 in SEQ ID NO: 3; R, K, D, Q at a residue corresponding to position 155 in SEQ ID NO: 3; I at a residue corresponding to position 156 in SEQ ID NO: 3; A, I, F at a residue corresponding to position 157 in SEQ ID NO: 3; E, V, K at a residue corresponding to position 158 in SEQ ID NO: 3; K, Q, D, E at a residue corresponding to position 159 in SEQ ID NO: 3; V, C, A at a residue corresponding to position 160 in SEQ ID NO: 3; I, A, F, Y, L, V at a residue corresponding to position 162 in SEQ ID NO: 3; D, A, P at a residue corresponding to position 164 in SEQ ID NO: 3; H, G, D at a residue corresponding to position 165 in SEQ ID NO: 3; C, A, I, V at a residue corresponding to position 166 in SEQ ID NO: 3; V, F, L at a residue corresponding to position 167 in SEQ ID NO: 3; T, N at a residue corresponding to position 168 in SEQ ID NO: 3; L, W, C, M, I at a residue corresponding to position 169 in SEQ ID NO: 3; L, V at a residue corresponding to position 170 in SEQ ID NO: 3; E, P, N, D at a residue corresponding to position 171 in SEQ ID NO: 3; D, G, H at a residue corresponding to position 172 in SEQ ID NO: 3; G, D at a residue corresponding to position 173 in SEQ ID NO: 3; G, D, T at a residue corresponding to position 174 in SEQ ID NO: 3; I, H, R at a residue corresponding to position 175 in SEQ ID NO: 3; Q, D at a residue corresponding to position 176 in SEQ ID NO: 3; V, T, A, I at a residue corresponding to position 177 in SEQ ID NO: 3; V, A, T, G at a residue corresponding to position 178 in SEQ ID NO: 3; D, N, E at a residue corresponding to position 179 in SEQ ID NO: 3; H, Q, A, E at a residue corresponding to position 180 in SEQ ID NO: 3; I, F at a residue corresponding to position 181 in SEQ ID NO: 3; L, H, I, V at a residue corresponding to position 182 in SEQ ID NO: 3; Q, K, D, A, H, E at a residue corresponding to position 183 in SEQ ID NO: 3; H, P, S, G at a residue corresponding to position 184 in SEQ ID NO: 3; C, I at a residue corresponding to position 185 in SEQ ID NO: 3; R at a residue corresponding to position 186 in SEQ ID NO: 3; H, K, F, I, V, M at a residue corresponding to position 187 in SEQ ID NO: 3; I, L, V at a residue corresponding to position 188 in SEQ ID NO: 3; S, F, T, M, V, L at a residue corresponding to position 189 in SEQ ID NO: 3; P, A, F at a residue corresponding to position 190 in SEQ ID NO: 3; R at a residue corresponding to position 191 in SEQ ID NO: 3; S at a residue corresponding to position 193 in SEQ ID NO: 3; T, R, K, A at a residue corresponding to position 194 in SEQ ID NO: 3; A at a residue corresponding to position 195 in SEQ ID NO: 3; M, I, V at a residue corresponding to position 196 in SEQ ID NO: 3; I, M, G, A at a residue corresponding to position 197 in SEQ ID NO: 3; Q, E, R at a residue corresponding to position 198 in SEQ ID NO: 3; S, V at a residue corresponding to position 199 in SEQ ID NO: 3; C, I, V at a residue corresponding to position 200 in SEQ ID NO: 3; M, Q, A at a residue corresponding to position 201 in SEQ ID NO: 3; N, K, A, R, T, E, Q at a residue corresponding to position 202 in SEQ ID NO: 3; N, H, A at a residue corresponding to position 203 in SEQ ID NO: 3; R, T at a residue corresponding to position 205 in SEQ ID NO: 3; V, R, K at a residue corresponding to position 206 in SEQ ID NO: 3; V, S, C, T at a residue corresponding to position 207 in SEQ ID NO: 3; V, T, Y, M, L at a residue corresponding to position 208 in SEQ ID NO: 3; L, E, M at a residue corresponding to position 209 in SEQ ID NO: 3; H, L at a residue corresponding to position 210 in SEQ ID NO: 3; L at a residue corresponding to position 211 in SEQ ID NO: 3; A, P, G at a residue corresponding to position 212 in SEQ ID NO: 3; K at a residue corresponding to position 213 in SEQ ID NO: 3; S, K at a residue corresponding to position 214 in SEQ ID NO: 3; S, N, A, C, D, P at a residue corresponding to position 215 in SEQ ID NO: 3; H, A, P at a residue corresponding to position 216 in SEQ ID NO: 3; F, A, I, C, M at a residue corresponding to position 217 in SEQ ID NO: 3; I, V, F, Y at a residue corresponding to position 218 in SEQ ID NO: 3; M, F, L, I at a residue corresponding to position 219 in SEQ ID NO: 3; C, F at a residue corresponding to position 220 in SEQ ID NO: 3; D, P at a residue corresponding to position 221 in SEQ ID NO: 3; T, S at a residue corresponding to position 222 in SEQ ID NO: 3; C at a residue corresponding to position 223 in SEQ ID NO: 3; N at a residue corresponding to position 224 in SEQ ID NO: 3; V at a residue corresponding to position 225 in SEQ ID NO: 3; E at a residue corresponding to position 226 in SEQ ID NO: 3; K, R, M, E at a residue corresponding to position 227 in SEQ ID NO: 3; A at a residue corresponding to position 228 in SEQ ID NO: 3; W, V at a residue corresponding to position 229 in SEQ ID NO: 3; R, D, P, Q, K, E at a residue corresponding to position 230 in SEQ ID NO: 3; Q, R, I, K at a residue corresponding to position 231 in SEQ ID NO: 3; I, F, T, C, Q, M, N at a residue corresponding to position 232 in SEQ ID NO: 3; V, L, M at a residue corresponding to position 233 in SEQ ID NO: 3; W, Q, F, A, M at a residue corresponding to position 234 in SEQ ID NO: 3; A, S at a residue corresponding to position 235 in SEQ ID NO: 3; K, C, W, A, I at a residue corresponding to position 236 in SEQ ID NO: 3; F, Y, C, M at a residue corresponding to position 237 in SEQ ID NO: 3; R, M, Q, Y, W at a residue corresponding to position 238 in SEQ ID NO: 3; R, H, Q at a residue corresponding to position 239 in SEQ ID NO: 3; Q, A, T, C, S at a residue corresponding to position 240 in SEQ ID NO: 3; N at a residue corresponding to position 241 in SEQ ID NO: 3; V, M, T, C at a residue corresponding to position 243 in SEQ ID NO: 3; M, I, N, S, V, A, C at a residue corresponding to position 245 in SEQ ID NO: 3; S, A at a residue corresponding to position 246 in SEQ ID NO: 3; G, P, I, A at a residue corresponding to position 247 in SEQ ID NO: 3; D, T, E, K, S, N at a residue corresponding to position 248 in SEQ ID NO: 3; Y, R at a residue corresponding to position 249 in SEQ ID NO: 3; I, L, M at a residue corresponding to position 250 in SEQ ID NO: 3; Y, F, L, V at a residue corresponding to position 251 in SEQ ID NO: 3; V, I at a residue corresponding to position 252 in SEQ ID NO: 3; D, Q, H, V, E at a residue corresponding to position 253 in SEQ ID NO: 3; Q, K, R, D at a residue corresponding to position 254 in SEQ ID NO: 3; E at a residue corresponding to position 255 in SEQ ID NO: 3; V, M, I, R at a residue corresponding to position 256 in SEQ ID NO: 3; A, F, H, Y at a residue corresponding to position 257 in SEQ ID NO: 3; D at a residue corresponding to position 258 in SEQ ID NO: 3; A, R, K, D, Q at a residue corresponding to position 259 in SEQ ID NO: 3; I, V, F, L at a residue corresponding to position 260 in SEQ ID NO: 3; L, M, V at a residue corresponding to position 261 in SEQ ID NO: 3; Q, D, E, K at a residue corresponding to position 262 in SEQ ID NO: 3; E, K, R at a residue corresponding to position 263 in SEQ ID NO: 3; M, V, F at a residue corresponding to position 264 in SEQ ID NO: 3; Q, R, A, E at a residue corresponding to position 265 in SEQ ID NO: 3; A, E at a residue corresponding to position 266 in SEQ ID NO: 3; Y, R, N, A, Q at a residue corresponding to position 267 in SEQ ID NO: 3; A at a residue corresponding to position 268 in SEQ ID NO: 3; S, G, C at a residue corresponding to position 269 in SEQ ID NO: 3; L, F, I, M, W at a residue corresponding to position 270 in SEQ ID NO: 3; F, K, V, W, R, H at a residue corresponding to position 271 in SEQ ID NO: 3; V, M, L at a residue corresponding to position 272 in SEQ ID NO: 3; C at a residue corresponding to position 273 in SEQ ID NO: 3; E, D at a residue corresponding to position 274 in SEQ ID NO: 3; P, G, A, E at a residue corresponding to position 275 in SEQ ID NO: 3; Q, M, R, D at a residue corresponding to position 276 in SEQ ID NO: 3; Q, E at a residue corresponding to position 277 in SEQ ID NO: 3; P, E at a residue corresponding to position 278 in SEQ ID NO: 3; E, D, S at a residue corresponding to position 279 in SEQ ID NO: 3; Q, R, T at a residue corresponding to position 280 in SEQ ID NO: 3; V, M, I at a residue corresponding to position 281 in SEQ ID NO: 3; Q, K, M, G, E at a residue corresponding to position 282 in SEQ ID NO: 3; E, H, N at a residue corresponding to position 283 in SEQ ID NO: 3; V, P at a residue corresponding to position 284 in SEQ ID NO: 3; L, V, I, M at a residue corresponding to position 285 in SEQ ID NO: 3; L, I, W, Y, V at a residue corresponding to position 286 in SEQ ID NO: 3; R, D, E at a residue corresponding to position 288 in SEQ ID NO: 3; M at a residue corresponding to position 289 in SEQ ID NO: 3; H at a residue corresponding to position 290 in SEQ ID NO: 3; K, L, E, T, H, A, N at a residue corresponding to position 291 in SEQ ID NO: 3; I, M at a residue corresponding to position 292 in SEQ ID NO: 3; A, V, I, N at a residue corresponding to position 293 in SEQ ID NO: 3; S, N, P, E, R at a residue corresponding to position 294 in SEQ ID NO: 3; R, K, A, Q, D at a residue corresponding to position 295 in SEQ ID NO: 3; W, R, V, F, H, L at a residue corresponding to position 296 in SEQ ID NO: 3; A, T, Q, V, L, H, I at a residue corresponding to position 297 in SEQ ID NO: 3; A at a residue corresponding to position 298 in SEQ ID NO: 3; K, Q, R at a residue corresponding to position 299 in SEQ ID NO: 3; A, R at a residue corresponding to position 300 in SEQ ID NO: 3; I, A at a residue corresponding to position 301 in SEQ ID NO: 3; A, R, D, Q, E at a residue corresponding to position 302 in SEQ ID NO: 3; Q, W, R, E at a residue corresponding to position 303 in SEQ ID NO: 3; L, G, Y at a residue corresponding to position 304 in SEQ ID NO: 3; I, V at a residue corresponding to position 305 in SEQ ID NO: 3; H, K, E, R at a residue corresponding to position 306 in SEQ ID NO: 3; I, V, M at a residue corresponding to position 307 in SEQ ID NO: 3; A, T at a residue corresponding to position 308 in SEQ ID NO: 3; G, V, L, F at a residue corresponding to position 310 in SEQ ID NO: 3; R, T, K, S, D, E at a residue corresponding to position 311 in SEQ ID NO: 3; A, D, T, K at a residue corresponding to position 312 in SEQ ID NO: 3; L, V at a residue corresponding to position 313 in SEQ ID NO: 3; A at a residue corresponding to position 314 in SEQ ID NO: 3; D at a residue corresponding to position 315 in SEQ ID NO: 3; E, G, N at a residue corresponding to position 316 in SEQ ID NO: 3; G, T at a residue corresponding to position 317 in SEQ ID NO: 3; R, T, Y at a residue corresponding to position 318 in SEQ ID NO: 3; F, L, M, I, Y at a residue corresponding to position 319 in SEQ ID NO: 3; Y, L at a residue corresponding to position 320 in SEQ ID NO: 3; L, P, C, I at a residue corresponding to position 321 in SEQ ID NO: 3; A, T, G, V at a residue corresponding to position 322 in SEQ ID NO: 3; E, L, I, G, P, V, K at a residue corresponding to position 323 in SEQ ID NO: 3; I, V, L at a residue corresponding to position 324 in SEQ ID NO: 3; K, E, D, T at a residue corresponding to position 325 in SEQ ID NO: 3; D at a residue corresponding to position 326 in SEQ ID NO: 3; W, F, V at a residue corresponding to position 327 in SEQ ID NO: 3; D, T, S at a residue corresponding to position 328 in SEQ ID NO: 3; E, R, P, D, N at a residue corresponding to position 329 in SEQ ID NO: 3; E at a residue corresponding to position 330 in SEQ ID NO: 3; S, D, H, M, Y at a residue corresponding to position 331 in SEQ ID NO: 3; R, T at a residue corresponding to position 332 in SEQ ID NO: 3; M, V, F, I at a residue corresponding to position 333 in SEQ ID NO: 3; V, S, M, C at a residue corresponding to position 334 in SEQ ID NO: 3; R, Q, H at a residue corresponding to position 335 in SEQ ID NO: 3; Q, E at a residue corresponding to position 337 in SEQ ID NO: 3; F, V, I, M at a residue corresponding to position 338 in SEQ ID NO: 3; C, M at a residue corresponding to position 339 in SEQ ID NO: 3; I at a residue corresponding to position 341 in SEQ ID NO: 3; M, V, I at a residue corresponding to position 342 in SEQ ID NO: 3; C, T, V, S, P at a residue corresponding to position 343 in SEQ ID NO: 3; I, V, L, M at a residue corresponding to position 344 in SEQ ID NO: 3; Y, V, M, L, I at a residue corresponding to position 345 in SEQ ID NO: 3; R, K, P at a residue corresponding to position 346 in SEQ ID NO: 3; F, V at a residue corresponding to position 347 in SEQ ID NO: 3; E, K, S at a residue corresponding to position 348 in SEQ ID NO: 3; S, G, D, N, T at a residue corresponding to position 349 in SEQ ID NO: 3; V, E, I, L, F at a residue corresponding to position 350 in SEQ ID NO: 3; D at a residue corresponding to position 351 in SEQ ID NO: 3; Q, H, E at a residue corresponding to position 352 in SEQ ID NO: 3; L, V, C at a residue corresponding to position 354 in SEQ ID NO: 3; R, A, D, Q, S, E at a residue corresponding to position 355 in SEQ ID NO: 3; M, L, V at a residue corresponding to position 356 in SEQ ID NO: 3; C at a residue corresponding to position 357 in SEQ ID NO: 3; N, R at a residue corresponding to position 358 in SEQ ID NO: 3; A, D, S, N, E at a residue corresponding to position 359 in SEQ ID NO: 3; S, T at a residue corresponding to position 360 in SEQ ID NO: 3; D, V at a residue corresponding to position 361 in SEQ ID NO: 3; L, T, N, S at a residue corresponding to position 362 in SEQ ID NO: 3; Y, E at a residue corresponding to position 363 in SEQ ID NO: 3; Q, Y, F, H at a residue corresponding to position 364 in SEQ ID NO: 3; Y, K, F, A, L at a residue corresponding to position 366 in SEQ ID NO: 3; L, A at a residue corresponding to position 368 in SEQ ID NO: 3; T, A at a residue corresponding to position 369 in SEQ ID NO: 3; C, A at a residue corresponding to position 370 in SEQ ID NO: 3; M, I, A, W, S, Y at a residue corresponding to position 371 in SEQ ID NO: 3; V, M at a residue corresponding to position 372 in SEQ ID NO: 3; W, Y, F at a residue corresponding to position 373 in SEQ ID NO: 3; T at a residue corresponding to position 374 in SEQ ID NO: 3; H, Q, K, T, R at a residue corresponding to position 375 in SEQ ID NO: 3; N, S, D at a residue corresponding to position 376 in SEQ ID NO: 3; D, I, V, E at a residue corresponding to position 377 in SEQ ID NO: 3; K, D, A at a residue corresponding to position 378 in SEQ ID NO: 3; N, K, V, R at a residue corresponding to position 379 in SEQ ID NO: 3; A, M, V at a residue corresponding to position 380 in SEQ ID NO: 3; M, W, K, R, T, H at a residue corresponding to position 381 in SEQ ID NO: 3; K, R, E, Q at a residue corresponding to position 382 in SEQ ID NO: 3; M, L, Y, F, I at a residue corresponding to position 383 in SEQ ID NO: 3; S, G at a residue corresponding to position 384 in SEQ ID NO: 3; Q, L, R, K at a residue corresponding to position 385 in SEQ ID NO: 3; E, Q, K, R at a residue corresponding to position 386 in SEQ ID NO: 3; M, V, L at a residue corresponding to position 387 in SEQ ID NO: 3; R, H, D, Q, E at a residue corresponding to position 388 in SEQ ID NO: 3; A, C, T at a residue corresponding to position 389 in SEQ ID NO: 3; T, N, C, G at a residue corresponding to position 390 in SEQ ID NO: 3; M, T, I at a residue corresponding to position 391 in SEQ ID NO: 3; V, I, M at a residue corresponding to position 392 in SEQ ID NO: 3; M, I, W, L, G at a residue corresponding to position 393 in SEQ ID NO: 3; I, K at a residue corresponding to position 394 in SEQ ID NO: 3; R, E, S, D at a residue corresponding to position 396 in SEQ ID NO: 3; I, W, A, P, F, Y at a residue corresponding to position 397 in SEQ ID NO: 3; S, C, P, T at a residue corresponding to position 398 in SEQ ID NO: 3; A, S at a residue corresponding to position 399 in SEQ ID NO: 3; I, S, F, V, H at a residue corresponding to position 400 in SEQ ID NO: 3; G, H, D, S, A at a residue corresponding to position 401 in SEQ ID NO: 3; N, A, F, M at a residue corresponding to position 402 in SEQ ID NO: 3; S at a residue corresponding to position 403 in SEQ ID NO: 3; I, F, V, G at a residue corresponding to position 405 in SEQ ID NO: 3; Y, K, F, L, W at a residue corresponding to position 406 in SEQ ID NO: 3; H, Q, Y, E at a residue corresponding to position 407 in SEQ ID NO: 3; T, S, H at a residue corresponding to position 408 in SEQ ID NO: 3; F, I, M at a residue corresponding to position 410 in SEQ ID NO: 3; T, G, P, A at a residue corresponding to position 411 in SEQ ID NO: 3; F, R at a residue corresponding to position 412 in SEQ ID NO: 3; A at a residue corresponding to position 413 in SEQ ID NO: 3; M, F, V at a residue corresponding to position 414 in SEQ ID NO: 3; P, S at a residue corresponding to position 415 in SEQ ID NO: 3; M, F, I at a residue corresponding to position 416 in SEQ ID NO: 3; F, I, T at a residue corresponding to position 418 in SEQ ID NO: 3; S, R at a residue corresponding to position 420 in SEQ ID NO: 3; Y, F at a residue corresponding to position 421 in SEQ ID NO: 3; K, R, G at a residue corresponding to position 423 in SEQ ID NO: 3; S, R at a residue corresponding to position 424 in SEQ ID NO: 3; I, G, W, A at a residue corresponding to position 425 in SEQ ID NO: 3; V, T, L at a residue corresponding to position 426 in SEQ ID NO: 3; G, T, A at a residue corresponding to position 427 in SEQ ID NO: 3; D, H at a residue corresponding to position 428 in SEQ ID NO: 3; E at a residue corresponding to position 429 in SEQ ID NO: 3; I, Y, L, V at a residue corresponding to position 430 in SEQ ID NO: 3; G, S, N at a residue corresponding to position 431 in SEQ ID NO: 3; A, P, F, W at a residue corresponding to position 432 in SEQ ID NO: 3; T, R, K, H, M, E at a residue corresponding to position 433 in SEQ ID NO: 3; R, Q, D, N at a residue corresponding to position 434 in SEQ ID NO: 3; V, F, W, Y, M at a residue corresponding to position 435 in SEQ ID NO: 3; T, L, F, I, V at a residue corresponding to position 436 in SEQ ID NO: 3; H, Q, Y at a residue corresponding to position 437 in SEQ ID NO: 3; V, T, W, C, Y, I at a residue corresponding to position 438 in SEQ ID NO: 3; Q, S, R, K at a residue corresponding to position 439 in SEQ ID NO: 3; K, W at a residue corresponding to position 440 in SEQ ID NO: 3; L, I, M at a residue corresponding to position 441 in SEQ ID NO: 3; G, A at a residue corresponding to position 442 in SEQ ID NO: 3; H, W, R, F at a residue corresponding to position 443 in SEQ ID NO: 3; E, P, K at a residue corresponding to position 444 in SEQ ID NO: 3; I, Y, F, M, L at a residue corresponding to position 445 in SEQ ID NO: 3; E, K at a residue corresponding to position 446 in SEQ ID NO: 3; E, N at a residue corresponding to position 447 in SEQ ID NO: 3; W, K, P at a residue corresponding to position 448 in SEQ ID NO: 3; E, R, F, H at a residue corresponding to position 449 in SEQ ID NO: 3; M, C, F, V, L at a residue corresponding to position 450 in SEQ ID NO: 3; W at a residue corresponding to position 451 in SEQ ID NO: 3; S, D, K at a residue corresponding to position 452 in SEQ ID NO: 3; E, P, H at a residue corresponding to position 453 in SEQ ID NO: 3; A, D at a residue corresponding to position 454 in SEQ ID NO: 3; K, Q, D at a residue corresponding to position 455 in SEQ ID NO: 3; F, L, V, W, M at a residue corresponding to position 456 in SEQ ID NO: 3; M, Y, F at a residue corresponding to position 457 in SEQ ID NO: 3; H, G at a residue corresponding to position 458 in SEQ ID NO: 3; D at a residue corresponding to position 459 in SEQ ID NO: 3; or any combination thereof. It is understood that when alterations are listed herein for a certain position, the alteration can be in the alternative or in the conjunctive depending on the context. For example, the K, Q, D at a residue corresponding to position 455 in SEQ ID NO: 3, means that alterations at a residue corresponding to position 471 in SEQ ID NO: 3 can comprise a K or Q or D.

In some embodiments, the one or more amino acid alterations of the engineered ALDH is an alteration described in TABLE 12, which results in the engineered ALDH having greater or similar activity on HHA-CoA, and similar or lower activity on succinyl-CoA, as compared to the WT, when the engineered ALDH is expressed in an organism having a pathway for production of HHA-CoA and assayed under conditions as described in Example 8. For example, in some embodiments, the one or more amino acid alterations result in an engineered ALDH having: Q, T, P at a residue corresponding to position 2 in SEQ ID NO: 3; S, K at a residue corresponding to position 3 in SEQ ID NO: 3; G, S at a residue corresponding to position 4 in SEQ ID NO: 3; I, T at a residue corresponding to position 5 in SEQ ID NO: 3; F at a residue corresponding to position 6 in SEQ ID NO: 3; E, Q at a residue corresponding to position 7 in SEQ ID NO: 3; V at a residue corresponding to position 9 in SEQ ID NO: 3; E, D, Q, R at a residue corresponding to position 11 in SEQ ID NO: 3; L, A at a residue corresponding to position 12 in SEQ ID NO: 3; R, K at a residue corresponding to position 15 in SEQ ID NO: 3; A at a residue corresponding to position 16 in SEQ ID NO: 3; K at a residue corresponding to position 17 in SEQ ID NO: 3; K, E, Q at a residue corresponding to position 18 in SEQ ID NO: 3; F, H at a residue corresponding to position 20 in SEQ ID NO: 3; A, R at a residue corresponding to position 21 in SEQ ID NO: 3; K, Q, A at a residue corresponding to position 22 in SEQ ID NO: 3; Y at a residue corresponding to position 23 in SEQ ID NO: 3; E, K at a residue corresponding to position 24 in SEQ ID NO: 3; A, E at a residue corresponding to position 25 in SEQ ID NO: 3; L, M, W at a residue corresponding to position 26 in SEQ ID NO: 3; D, T at a residue corresponding to position 27 in SEQ ID NO: 3; W at a residue corresponding to position 28 in SEQ ID NO: 3; D, K, Q at a residue corresponding to position 30 in SEQ ID NO: 3; I, L at a residue corresponding to position 31 in SEQ ID NO: 3; N, E at a residue corresponding to position 32 in SEQ ID NO: 3; E, Q, A, R at a residue corresponding to position 33 in SEQ ID NO: 3; V at a residue corresponding to position 34 in SEQ ID NO: 3; C, M at a residue corresponding to position 35 in SEQ ID NO: 3; K at a residue corresponding to position 36 in SEQ ID NO: 3; K, A, R at a residue corresponding to position 37 in SEQ ID NO: 3; L, I at a residue corresponding to position 38 in SEQ ID NO: 3; A at a residue corresponding to position 39 in SEQ ID NO: 3; W at a residue corresponding to position 40 in SEQ ID NO: 3; L, V at a residue corresponding to position 41 in SEQ ID NO: 3; L, V, M, C at a residue corresponding to position 42 in SEQ ID NO: 3; E, L at a residue corresponding to position 43 in SEQ ID NO: 3; K, Q at a residue corresponding to position 44 in SEQ ID NO: 3; H, R at a residue corresponding to position 45 in SEQ ID NO: 3; D at a residue corresponding to position 47 in SEQ ID NO: 3; E, M, D at a residue corresponding to position 48 in SEQ ID NO: 3; I at a residue corresponding to position 49 in SEQ ID NO: 3; N, E, Q at a residue corresponding to position 51 in SEQ ID NO: 3; L at a residue corresponding to position 52 in SEQ ID NO: 3; L at a residue corresponding to position 54 in SEQ ID NO: 3; E, R at a residue corresponding to position 55 in SEQ ID NO: 3; R at a residue corresponding to position 58 in SEQ ID NO: 3; D, E, R at a residue corresponding to position 61 in SEQ ID NO: 3; F, V, W at a residue corresponding to position 62 in SEQ ID NO: 3; D, E, Q at a residue corresponding to position 63 in SEQ ID NO: 3; H at a residue corresponding to position 64 in SEQ ID NO: 3; R at a residue corresponding to position 65 in SEQ ID NO: 3; V at a residue corresponding to position 66 in SEQ ID NO: 3; E at a residue corresponding to position 67 in SEQ ID NO: 3; M, R at a residue corresponding to position 70 in SEQ ID NO: 3; D, E, Y, F, H at a residue corresponding to position 71 in SEQ ID NO: 3; T at a residue corresponding to position 73 in SEQ ID NO: 3; E, R at a residue corresponding to position 74 in SEQ ID NO: 3; G at a residue corresponding to position 75 in SEQ ID NO: 3; Y at a residue corresponding to position 77 in SEQ ID NO: 3; E, F, Y, H, N at a residue corresponding to position 78 in SEQ ID NO: 3; D, W at a residue corresponding to position 79 in SEQ ID NO: 3; F, V, I at a residue corresponding to position 80 in SEQ ID NO: 3; A, R at a residue corresponding to position 81 in SEQ ID NO: 3; G at a residue corresponding to position 82 in SEQ ID NO: 3; E at a residue corresponding to position 83 in SEQ ID NO: 3; R, Q at a residue corresponding to position 84 in SEQ ID NO: 3; V, K at a residue corresponding to position 86 in SEQ ID NO: 3; M at a residue corresponding to position 88 in SEQ ID NO: 3; V, L at a residue corresponding to position 89 in SEQ ID NO: 3; S, D, H, E, R at a residue corresponding to position 90 in SEQ ID NO: 3; E, Q at a residue corresponding to position 91 in SEQ ID NO: 3; D at a residue corresponding to position 92 in SEQ ID NO: 3; D at a residue corresponding to position 93 in SEQ ID NO: 3; G, D at a residue corresponding to position 94 in SEQ ID NO: 3; K, M, R at a residue corresponding to position 95 in SEQ ID NO: 3; N at a residue corresponding to position 96 in SEQ ID NO: 3; I, V, M at a residue corresponding to position 97 in SEQ ID NO: 3; Y, I, M, T at a residue corresponding to position 98 in SEQ ID NO: 3; R, T, Q, K at a residue corresponding to position 99 in SEQ ID NO: 3; Y, I, M, L at a residue corresponding to position 100 in SEQ ID NO: 3; Y, H at a residue corresponding to position 101 in SEQ ID NO: 3; R, Q at a residue corresponding to position 102 in SEQ ID NO: 3; F, M at a residue corresponding to position 104 in SEQ ID NO: 3; C, V at a residue corresponding to position 107 in SEQ ID NO: 3; T, S at a residue corresponding to position 111 in SEQ ID NO: 3; I, T, L at a residue corresponding to position 117 in SEQ ID NO: 3; T, Sat a residue corresponding to position 119 in SEQ ID NO: 3; A, T, M at a residue corresponding to position 120 in SEQ ID NO: 3; L, V, I at a residue corresponding to position 121 in SEQ ID NO: 3; H at a residue corresponding to position 122 in SEQ ID NO: 3; I, V, L, M at a residue corresponding to position 124 in SEQ ID NO: 3; I, L, V at a residue corresponding to position 125 in SEQ ID NO: 3; A at a residue corresponding to position 126 in SEQ ID NO: 3; I at a residue corresponding to position 128 in SEQ ID NO: 3; A, M at a residue corresponding to position 129 in SEQ ID NO: 3; A, S at a residue corresponding to position 130 in SEQ ID NO: 3; C, T at a residue corresponding to position 133 in SEQ ID NO: 3; L, I at a residue corresponding to position 136 in SEQ ID NO: 3; R at a residue corresponding to position 144 in SEQ ID NO: 3; C, T, V at a residue corresponding to position 145 in SEQ ID NO: 3; N at a residue corresponding to position 146 in SEQ ID NO: 3; A, I, M at a residue corresponding to position 147 in SEQ ID NO: 3; E, L, A, R at a residue corresponding to position 148 in SEQ ID NO: 3; M, V, L, I at a residue corresponding to position 149 in SEQ ID NO: 3; A, I, M at a residue corresponding to position 150 in SEQ ID NO: 3; K, D, Q at a residue corresponding to position 151 in SEQ ID NO: 3; M, I at a residue corresponding to position 152 in SEQ ID NO: 3; M, C, F, V at a residue corresponding to position 153 in SEQ ID NO: 3; Q, K at a residue corresponding to position 154 in SEQ ID NO: 3; K, R, D, Q at a residue corresponding to position 155 in SEQ ID NO: 3; I at a residue corresponding to position 156 in SEQ ID NO: 3; A, F, I at a residue corresponding to position 157 in SEQ ID NO: 3; E, V, K at a residue corresponding to position 158 in SEQ ID NO: 3; D, K at a residue corresponding to position 159 in SEQ ID NO: 3; V, A at a residue corresponding to position 160 in SEQ ID NO: 3; F, Y, V, A, L at a residue corresponding to position 162 in SEQ ID NO: 3; A, D, P at a residue corresponding to position 164 in SEQ ID NO: 3; D, H at a residue corresponding to position 165 in SEQ ID NO: 3; V, C, I at a residue corresponding to position 166 in SEQ ID NO: 3; L, V, F at a residue corresponding to position 167 in SEQ ID NO: 3; T, N at a residue corresponding to position 168 in SEQ ID NO: 3; C, L, M, I at a residue corresponding to position 169 in SEQ ID NO: 3; V at a residue corresponding to position 170 in SEQ ID NO: 3; D, E, P, N at a residue corresponding to position 171 in SEQ ID NO: 3; D, G, H at a residue corresponding to position 172 in SEQ ID NO: 3; T at a residue corresponding to position 174 in SEQ ID NO: 3; I, H at a residue corresponding to position 175 in SEQ ID NO: 3; D at a residue corresponding to position 176 in SEQ ID NO: 3; A, T, I at a residue corresponding to position 177 in SEQ ID NO: 3; E at a residue corresponding to position 180 in SEQ ID NO: 3; I at a residue corresponding to position 181 in SEQ ID NO: 3; Q, A, H, K at a residue corresponding to position 183 in SEQ ID NO: 3; G, S at a residue corresponding to position 184 in SEQ ID NO: 3; M, V, I, F, K at a residue corresponding to position 187 in SEQ ID NO: 3; V, L at a residue corresponding to position 189 in SEQ ID NO: 3; A at a residue corresponding to position 190 in SEQ ID NO: 3; A, T, R, K at a residue corresponding to position 194 in SEQ ID NO: 3; V at a residue corresponding to position 196 in SEQ ID NO: 3; I at a residue corresponding to position 197 in SEQ ID NO: 3; R at a residue corresponding to position 198 in SEQ ID NO: 3; V at a residue corresponding to position 199 in SEQ ID NO: 3; C at a residue corresponding to position 200 in SEQ ID NO: 3; A at a residue corresponding to position 201 in SEQ ID NO: 3; A, T, R at a residue corresponding to position 202 in SEQ ID NO: 3; N, H, A at a residue corresponding to position 203 in SEQ ID NO: 3; V at a residue corresponding to position 206 in SEQ ID NO: 3; V, T, C at a residue corresponding to position 207 in SEQ ID NO: 3; V, M, Y at a residue corresponding to position 208 in SEQ ID NO: 3; A, P at a residue corresponding to position 212 in SEQ ID NO: 3; S at a residue corresponding to position 214 in SEQ ID NO: 3; P, N, C, A, S at a residue corresponding to position 215 in SEQ ID NO: 3; A, P at a residue corresponding to position 216 in SEQ ID NO: 3; M, A, C at a residue corresponding to position 217 in SEQ ID NO: 3; I, V at a residue corresponding to position 218 in SEQ ID NO: 3; I, M, L at a residue corresponding to position 219 in SEQ ID NO: 3; P, D at a residue corresponding to position 221 in SEQ ID NO: 3; T, S at a residue corresponding to position 222 in SEQ ID NO: 3; C at a residue corresponding to position 223 in SEQ ID NO: 3; N at a residue corresponding to position 224 in SEQ ID NO: 3; V at a residue corresponding to position 225 in SEQ ID NO: 3; M, E, K at a residue corresponding to position 227 in SEQ ID NO: 3; A at a residue corresponding to position 228 in SEQ ID NO: 3; V at a residue corresponding to position 229 in SEQ ID NO: 3; D, E, P, K at a residue corresponding to position 230 in SEQ ID NO: 3; R, Q, I, K at a residue corresponding to position 231 in SEQ ID NO: 3; M at a residue corresponding to position 232 in SEQ ID NO: 3; L at a residue corresponding to position 233 in SEQ ID NO: 3; F, A, W, M, Q at a residue corresponding to position 234 in SEQ ID NO: 3; F, Y, C, M at a residue corresponding to position 237 in SEQ ID NO: 3; R, Q, M at a residue corresponding to position 238 in SEQ ID NO: 3; T at a residue corresponding to position 240 in SEQ ID NO: 3; V at a residue corresponding to position 243 in SEQ ID NO: 3; I at a residue corresponding to position 245 in SEQ ID NO: 3; S, A at a residue corresponding to position 246 in SEQ ID NO: 3; N at a residue corresponding to position 248 in SEQ ID NO: 3; Y at a residue corresponding to position 249 in SEQ ID NO: 3; M at a residue corresponding to position 250 in SEQ ID NO: 3; F, L at a residue corresponding to position 251 in SEQ ID NO: 3; V, I at a residue corresponding to position 252 in SEQ ID NO: 3; H at a residue corresponding to position 253 in SEQ ID NO: 3; K, R, D at a residue corresponding to position 254 in SEQ ID NO: 3; E at a residue corresponding to position 255 in SEQ ID NO: 3; I, M, V, R at a residue corresponding to position 256 in SEQ ID NO: 3; Y, A at a residue corresponding to position 257 in SEQ ID NO: 3; D at a residue corresponding to position 258 in SEQ ID NO: 3; R, A, Q, D at a residue corresponding to position 259 in SEQ ID NO: 3; V, L at a residue corresponding to position 260 in SEQ ID NO: 3; V, L, M at a residue corresponding to position 261 in SEQ ID NO: 3; K at a residue corresponding to position 262 in SEQ ID NO: 3; K, R at a residue corresponding to position 263 in SEQ ID NO: 3; V, F at a residue corresponding to position 264 in SEQ ID NO: 3; R, Q, E at a residue corresponding to position 265 in SEQ ID NO: 3; E at a residue corresponding to position 266 in SEQ ID NO: 3; R at a residue corresponding to position 267 in SEQ ID NO: 3; F, L, W, M, I at a residue corresponding to position 270 in SEQ ID NO: 3; W, F, K, H at a residue corresponding to position 271 in SEQ ID NO: 3; V, L, M at a residue corresponding to position 272 in SEQ ID NO: 3; C at a residue corresponding to position 273 in SEQ ID NO: 3; D, E at a residue corresponding to position 274 in SEQ ID NO: 3; A, P, G, E at a residue corresponding to position 275 in SEQ ID NO: 3; R, D at a residue corresponding to position 276 in SEQ ID NO: 3; E, Q at a residue corresponding to position 277 in SEQ ID NO: 3; E at a residue corresponding to position 278 in SEQ ID NO: 3; S, E at a residue corresponding to position 279 in SEQ ID NO: 3; T at a residue corresponding to position 280 in SEQ ID NO: 3; I, V, M at a residue corresponding to position 281 in SEQ ID NO: 3; K, Q, G, M at a residue corresponding to position 282 in SEQ ID NO: 3; E, N, H at a residue corresponding to position 283 in SEQ ID NO: 3; V at a residue corresponding to position 284 in SEQ ID NO: 3; V at a residue corresponding to position 285 in SEQ ID NO: 3; Y, V, I at a residue corresponding to position 286 in SEQ ID NO: 3; E at a residue corresponding to position 288 in SEQ ID NO: 3; H at a residue corresponding to position 290 in SEQ ID NO: 3; L, A, T, H, N, K at a residue corresponding to position 291 in SEQ ID NO: 3; A, I, N at a residue corresponding to position 293 in SEQ ID NO: 3; R at a residue corresponding to position 294 in SEQ ID NO: 3; D, R, Q at a residue corresponding to position 295 in SEQ ID NO: 3; L, W at a residue corresponding to position 296 in SEQ ID NO: 3; K, Q, R at a residue corresponding to position 299 in SEQ ID NO: 3; A at a residue corresponding to position 300 in SEQ ID NO: 3; A at a residue corresponding to position 301 in SEQ ID NO: 3; A, E, Q at a residue corresponding to position 302 in SEQ ID NO: 3; E, R at a residue corresponding to position 303 in SEQ ID NO: 3; L at a residue corresponding to position 304 in SEQ ID NO: 3; V at a residue corresponding to position 305 in SEQ ID NO: 3; E, H, K, R at a residue corresponding to position 306 in SEQ ID NO: 3; I, M, V at a residue corresponding to position 307 in SEQ ID NO: 3; T at a residue corresponding to position 308 in SEQ ID NO: 3; F, V, L at a residue corresponding to position 310 in SEQ ID NO: 3; D, S, R, E at a residue corresponding to position 311 in SEQ ID NO: 3; A, T, D, K at a residue corresponding to position 312 in SEQ ID NO: 3; V, L at a residue corresponding to position 313 in SEQ ID NO: 3; D at a residue corresponding to position 315 in SEQ ID NO: 3; G, E, N at a residue corresponding to position 316 in SEQ ID NO: 3; G, T at a residue corresponding to position 317 in SEQ ID NO: 3; R at a residue corresponding to position 318 in SEQ ID NO: 3; L, M at a residue corresponding to position 319 in SEQ ID NO: 3; Y, L at a residue corresponding to position 320 in SEQ ID NO: 3; I, L, C at a residue corresponding to position 321 in SEQ ID NO: 3; G, V, A, T at a residue corresponding to position 322 in SEQ ID NO: 3; I, V, K, P at a residue corresponding to position 323 in SEQ ID NO: 3; I, V, L at a residue corresponding to position 324 in SEQ ID NO: 3; E, T at a residue corresponding to position 325 in SEQ ID NO: 3; D at a residue corresponding to position 326 in SEQ ID NO: 3; F, W, V at a residue corresponding to position 327 in SEQ ID NO: 3; S at a residue corresponding to position 328 in SEQ ID NO: 3; P, R, D, N at a residue corresponding to position 329 in SEQ ID NO: 3; E at a residue corresponding to position 330 in SEQ ID NO: 3; D at a residue corresponding to position 331 in SEQ ID NO: 3; T, R at a residue corresponding to position 332 in SEQ ID NO: 3; I, M, F at a residue corresponding to position 333 in SEQ ID NO: 3; S, V, C, M at a residue corresponding to position 334 in SEQ ID NO: 3; R at a residue corresponding to position 335 in SEQ ID NO: 3; M at a residue corresponding to position 339 in SEQ ID NO: 3; I at a residue corresponding to position 341 in SEQ ID NO: 3; I, M at a residue corresponding to position 342 in SEQ ID NO: 3; S, C, V at a residue corresponding to position 343 in SEQ ID NO: 3; M, I, V, L at a residue corresponding to position 344 in SEQ ID NO: 3; L, M, Y, V at a residue corresponding to position 345 in SEQ ID NO: 3; P at a residue corresponding to position 346 in SEQ ID NO: 3; F, V at a residue corresponding to position 347 in SEQ ID NO: 3; E, K, S at a residue corresponding to position 348 in SEQ ID NO: 3; G, T, N, S, D at a residue corresponding to position 349 in SEQ ID NO: 3; E, I, V, F, L at a residue corresponding to position 350 in SEQ ID NO: 3; D at a residue corresponding to position 351 in SEQ ID NO: 3; L, C at a residue corresponding to position 354 in SEQ ID NO: 3; D, A, Q, S, E at a residue corresponding to position 355 in SEQ ID NO: 3; L, V at a residue corresponding to position 356 in SEQ ID NO: 3; R, N at a residue corresponding to position 358 in SEQ ID NO: 3; S, A, N, E at a residue corresponding to position 359 in SEQ ID NO: 3; S at a residue corresponding to position 360 in SEQ ID NO: 3; S, N, T at a residue corresponding to position 362 in SEQ ID NO: 3; E at a residue corresponding to position 363 in SEQ ID NO: 3; A, K, Y, L at a residue corresponding to position 366 in SEQ ID NO: 3; C, A at a residue corresponding to position 370 in SEQ ID NO: 3; S, A at a residue corresponding to position 371 in SEQ ID NO: 3; V, M at a residue corresponding to position 372 in SEQ ID NO: 3; F, Y, W at a residue corresponding to position 373 in SEQ ID NO: 3; T at a residue corresponding to position 374 in SEQ ID NO: 3; H, T, K, R, Q at a residue corresponding to position 375 in SEQ ID NO: 3; D at a residue corresponding to position 376 in SEQ ID NO: 3; E, I, V at a residue corresponding to position 377 in SEQ ID NO: 3; K, A, D at a residue corresponding to position 378 in SEQ ID NO: 3; R, N, V at a residue corresponding to position 379 in SEQ ID NO: 3; V at a residue corresponding to position 380 in SEQ ID NO: 3; W, H, M, R, K, T at a residue corresponding to position 381 in SEQ ID NO: 3; K at a residue corresponding to position 382 in SEQ ID NO: 3; I, M at a residue corresponding to position 383 in SEQ ID NO: 3; G, S at a residue corresponding to position 384 in SEQ ID NO: 3; R, K at a residue corresponding to position 385 in SEQ ID NO: 3; Q, K, R at a residue corresponding to position 386 in SEQ ID NO: 3; M, V, L at a residue corresponding to position 387 in SEQ ID NO: 3; R, E, Q, H, D at a residue corresponding to position 388 in SEQ ID NO: 3; T, A, C at a residue corresponding to position 389 in SEQ ID NO: 3; G, T, C at a residue corresponding to position 390 in SEQ ID NO: 3; M, V, I at a residue corresponding to position 392 in SEQ ID NO: 3; G at a residue corresponding to position 393 in SEQ ID NO: 3; I at a residue corresponding to position 394 in SEQ ID NO: 3; E, D, S at a residue corresponding to position 396 in SEQ ID NO: 3; W, I, Y at a residue corresponding to position 397 in SEQ ID NO: 3; S, I, H at a residue corresponding to position 400 in SEQ ID NO: 3; D, G, S, H, A at a residue corresponding to position 401 in SEQ ID NO: 3; M at a residue corresponding to position 402 in SEQ ID NO: 3; V, G at a residue corresponding to position 405 in SEQ ID NO: 3; W, F, L at a residue corresponding to position 406 in SEQ ID NO: 3; H, Q at a residue corresponding to position 407 in SEQ ID NO: 3; M at a residue corresponding to position 410 in SEQ ID NO: 3; G, A, P, T at a residue corresponding to position 411 in SEQ ID NO: 3; V at a residue corresponding to position 414 in SEQ ID NO: 3; S at a residue corresponding to position 420 in SEQ ID NO: 3; F at a residue corresponding to position 421 in SEQ ID NO: 3; G, R at a residue corresponding to position 423 in SEQ ID NO: 3; I, A, G at a residue corresponding to position 425 in SEQ ID NO: 3; V at a residue corresponding to position 426 in SEQ ID NO: 3; A, T at a residue corresponding to position 427 in SEQ ID NO: 3; D at a residue corresponding to position 428 in SEQ ID NO: 3; N at a residue corresponding to position 431 in SEQ ID NO: 3; T, H, K, M, R at a residue corresponding to position 433 in SEQ ID NO: 3; M, V at a residue corresponding to position 435 in SEQ ID NO: 3; V, F, I at a residue corresponding to position 436 in SEQ ID NO: 3; Q at a residue corresponding to position 437 in SEQ ID NO: 3; I, T, Y, C at a residue corresponding to position 438 in SEQ ID NO: 3; S at a residue corresponding to position 439 in SEQ ID NO: 3; W, K at a residue corresponding to position 440 in SEQ ID NO: 3; M, I, L at a residue corresponding to position 441 in SEQ ID NO: 3; W, F, R at a residue corresponding to position 443 in SEQ ID NO: 3; K at a residue corresponding to position 444 in SEQ ID NO: 3; K at a residue corresponding to position 446 in SEQ ID NO: 3; E, N at a residue corresponding to position 447 in SEQ ID NO: 3; W, K, P at a residue corresponding to position 448 in SEQ ID NO: 3; R, H at a residue corresponding to position 449 in SEQ ID NO: 3; V at a residue corresponding to position 450 in SEQ ID NO: 3; S, K at a residue corresponding to position 452 in SEQ ID NO: 3; H, P at a residue corresponding to position 453 in SEQ ID NO: 3; A, D at a residue corresponding to position 454 in SEQ ID NO: 3; K, D at a residue corresponding to position 455 in SEQ ID NO: 3; F at a residue corresponding to position 457 in SEQ ID NO: 3; H, G at a residue corresponding to position 458 in SEQ ID NO: 3; D at a residue corresponding to position 459 in SEQ ID NO: 3; more than one of the foregoing; or any combination thereof. It is understood that when alterations are listed herein for a certain position, the alteration can be in the alternative or in the conjunctive depending on the context. For example, the K, D at a residue corresponding to position 455 in SEQ ID NO: 3, means that alterations at a residue corresponding to position 471 in SEQ ID NO: 3 can comprise a K or D.

In certain embodiments, the one or more amino acid alterations of the engineered ALDH is an alteration described in TABLE 12, which results in the engineered ALDH having about or greater than 2× the amount of activity on HHA-CoA, and similar or lower activity on succinyl-CoA, as compared to the WT, when the engineered ALDH is expressed in an organism having a pathway for production of HHA and assayed under conditions as described in Example 8. For example, in some embodiments, the one or more amino acid alterations result in an engineered ALDH having: V at a residue corresponding to position 42 in SEQ ID NO: 3; T, S at a residue corresponding to position 119 in SEQ ID NO: 3; I at a residue corresponding to position 149 in SEQ ID NO: 3; K at a residue corresponding to position 158 in SEQ ID NO: 3; T at a residue corresponding to position 174 in SEQ ID NO: 3; A at a residue corresponding to position 246 in SEQ ID NO: 3; D at a residue corresponding to position 259 in SEQ ID NO: 3; R at a residue corresponding to position 263 in SEQ ID NO: 3; R at a residue corresponding to position 299 in SEQ ID NO: 3; V at a residue corresponding to position 327 in SEQ ID NO: 3; S at a residue corresponding to position 348 in SEQ ID NO: 3; A at a residue corresponding to position 370 in SEQ ID NO: 3; I at a residue corresponding to position 392 in SEQ ID NO: 3; A at a residue corresponding to position 401 in SEQ ID NO: 3; T at a residue corresponding to position 427 in SEQ ID NO: 3; more than one of the foregoing; or any combination thereof.

In some embodiments, an engineered ALDH described herein has one or more amino acid alterations comprising: E2P, E2Q, E2T, Q3E, Q3K, Q3M, Q3R, Q3S, A4G, A4S, V5I, V5T, K6E, K6F, K6H, K6P, K6Q, K6R, D7E, D7Q, Y8D, L9I, L9V, D10N, K11A, K11D, K11E, K11Q, K11R, M12A, M12I, M12L, V13L, A14D, A14E, A14K, A14Q, A15K, A15R, S16A, R17H, R17K, R17Q, 118E, I18K, I18Q, I18T, Q20F, Q20H, Q20M, Q21A, Q21E, Q21K, Q21P, Q21R, E22A, E22K, E22Q, F23L, F23W, F23Y, A24E, A24K, A24R, A24S, T25A, T25E, T25K, T25N, T25S, Y26F, Y26L, Y26M, Y26T, Y26W, P27D, P27S, P27T, Q28W, E29K, E29Q, T30D, T30E, T30K, T30Q, T30R, V31I, V31L, V31R, D32A, D32E, D32N, K33A, K33E, K33Q, K33R, A34I, A34L, A34M, A34V, V35C, V35I, V35L, V35M, R36E, R36H, R36K, T37A, T37K, T37R, V38F, V38I, V38L, V38M, V38W, G39A, G39R, K40D, K40E, K40Q, K40R, K40W, A41L, A41V, I42C, I42L, I42M, I42V, Y43E, Y43L, Y43R, D44E, D44K, D44Q, D44R, N45H, N45Q, N45R, E47D, L48D, L48E, L48M, L48R, L49F, L49I, L49M, L49W, L49Y, A50G, H51A, H51E, H51K, H51N, H51Q, H51R, M52L, A53E, A53I, A53S, V54I, V54L, V54M, V54T, D55E, D55Q, D55R, K58E, K58G, K58Q, K58R, M59F, M59I, M59L, M59Y, G60A, N61D, N61E, N61K, N61R, Y62A, Y62F, Y62I, Y62M, Y62V, Y62W, A63D, A63E, A63Q, D64E, D64H, K65E, K65R, I66L, I66V, A67E, K68D, K68E, C69H, C69I, C69L, C69M, C69N, V70I, V70L, V70M, V70R, N71D, N71E, N71F, N71H, N71Y, K72A, K72R, S73A, S73T, K74D, K74E, K74R, S75G, S75P, S75Y, V76F, V76I, V76L, V76M, V76Y, W77F, W77Y, W78E, W78F, W78H, W78N, W78R, W78Y, R79D, R79S, R79W, R79Y, M80F, M80I, M80L, M80V, K81A, K81E, K81R, D82E, D82G, D82P, K83E, K83Q, K84Q, K84R, S85A, S85T, R86E, R86K, R86V, G87T, I88L, I88M, I88V, I89L, I89P, I89V, K90D, K90E, K90H, K90P, K90R, K90S, R91D, R91E, R91Q, I92D, I92M, P93D, P93E, E94A, E94D, E94G, E94N, L95K, L95M, L95N, L95R, G96H, G96K, G96N, G96R, L97I, L97M, L97V, V98I, V98M, V98T, V98Y, E99K, E99Q, E99R, E99T, V100Y, V100L, V100M, V100I, A101K, A101Y, A101R, A101H, K102E, K102Q, K102R, K102V, I104V, I104Y, I104F, I104L, I104M, V106I, I107C, I107V, G108A, G108C, C109I, C109A, C109M, V110M, V110L, V110I, A111T, A111V, A111Y, A111I, A111S, P112E, T113F, T113S, T113W, T113Y, T114N, N115W, N115R, N115H, N115Y, V117N, V117L, V117T, V117I, I118N, I118A, I118L, I118M, I118S, I118F, I118V, N119Q, N119I, N119L, N119M, N119T, N119S, V120T, V120A, V120P, V120M, M121V, M121A, M121L, M121C, M121I, Q122C, Q122F, Q122R, Q122Y, Q122H, Q122N, Q122S, Q122G, Q122V, Q122W, N123K, N123P, A124V, A124L, A124M, A124I, M125I, M125L, M125A, M125V, C126A, C126P, A127M, L128I, K129A, K129M, C130T, C130S, C130A, G131R, N132C, S133T, S133C, M134I, M134C, M134V, I135V, I135M, V136L, V136M, V136F, V136I, S137R, S137K, P138G, H139S, H139G, P140E, R141Q, R141K, R141Y, R141E, A142T, A142S, K143P, H144L, H144N, H144R, S145T, S145C, S145V, S146T, S146C, S146A, S146N, V147I, V147M, V147A, K148Y, K148L, K148A, K148E, K148H, K148R, T149V, T149I, T149L, T149M, T149F, V150I, V150M, V150G, V150A, E151D, E151K, E151R, E151Q, V152L, V152M, V152I, I153F, I153V, I153C, I153M, N154H, N154A, N154Q, N154K, N154R, E155R, E155K, E155D, E155Q, A156I, L157A, L157I, L157F, A158E, A158V, A158K, A159K, A159Q, A159D, A159E, L160V, L160C, L160A, M162I, M162A, M162F, M162Y, M162L, M162V, K164D, K164A, K164P, N165H, N165G, N165D, L166C, L166A, L166I, L166V, I167V, I167F, I167L, Q168T, Q168N, V169L, V169W, V169C, V169M, V169I, I170L, I170V, T171E, T171P, T171N, T171D, E172D, E172G, E172H, P173G, P173D, S174G, S174D, S174T, M175I, M175H, M175R, E176Q, E176D, L177V, L177T, L177A, L177I, S178V, S178A, S178T, S178G, A179D, A179N, A179E, G180H, G180Q, G180A, G180E, L181I, L181F, M182L, M182H, M182I, M182V, S183Q, S183K, S183D, S183A, S183H, S183E, A184H, A184P, A184S, A184G, V185C, V185I, D186R, L187H, L187K, L187F, L187I, L187V, L187M, C188I, C188L, C188V, I189S, I189F, I189T, I189M, I189V, I189L, C190P, C190A, C190F, T191R, G193S, P194T, P194R, P194K, P194A, G195A, L196M, L196I, L196V, V197I, V197M, V197G, V197A, K198Q, K198E, K198R, A199S, A199V, A200C, A200I, A200V, Y201M, Y201Q, Y201A, S202N, S202K, S202A, S202R, S202T, S202E, S202Q, S203N, S203H, S203A, K205R, K205T, P206V, P206R, P206K, A207V, A207S, A207C, A207T, I208V, I208T, I208Y, I208M, I208L, G209L, G209E, G209M, V210H, V210L, G211L, Q212A, Q212P, Q212G, G213K, N214S, N214K, V215S, V215N, V215A, V215C, V215D, V215P, Q216H, Q216A, Q216P, V217F, V217A, V217I, V217C, V217M, L218I, L218V, L218F, L218Y, V219M, V219F, V219L, V219I, D220C, D220F, R221D, R221P, D222T, D222S, A223C, D224N, L225V, D226E, Q227K, Q227R, Q227M, Q227E, V228A, A229W, A229V, A230R, A230D, A230P, A230Q, A230K, A230E, M231Q, M231R, M231I, M231K, V232I, V232F, V232T, V232C, V232Q, V232M, V232N, I233V, I233L, I233M, K234W, K234Q, K234F, K234A, K234M, G235A, G235S, R236K, R236C, R236W, R236A, R236I, T237F, T237Y, T237C, T237M, F238R, F238M, F238Q, F238Y, F238W, D239R, D239H, D239Q, N240Q, N240A, N240T, N240C, N240S, G241N, L243V, L243M, L243T, L243C, T245M, T245I, T245N, T245S, T245V, T245A, T245C, C246S, C246A, E247G, E247P, E247I, E247A, Q248D, Q248T, Q248E, Q248K, Q248S, Q248N, N249Y, N249R, V250I, V250L, V250M, I251Y, I251F, I251L, I251V, C252V, C252I, P253D, P253Q, P253H, P253V, P253E, E254Q, E254K, E254R, E254D, D255E, K256V, K256M, K256I, K256R, K257A, K257F, K257H, K257Y, E258D, E259A, E259R, E259K, E259D, E259Q, M260I, M260V, M260F, M260L, I261L, I261M, I261V, A262Q, A262D, A262E, A262K, A263E, A263K, A263R, L264M, L264V, L264F, K265Q, K265R, K265A, K265E, K266A, K266E, H267Y, H267R, H267N, H267A, H267Q, G268A, A269S, A269G, A269C, Y270L, Y270F, Y270I, Y270M, Y270W, Y271F, Y271K, Y271V, Y271W, Y271R, Y271H, I272V, I272M, I272L, G273C, N274E, N274D, S275P, S275G, S275A, S275E, E276Q, E276M, E276R, E276D, D277Q, D277E, A278P, A278E, A279E, A279D, A279S, K280Q, K280R, K280T, L281V, L281M, L281I, R282Q, R282K, R282M, R282G, R282E, D283E, D283H, D283N, T284V, T284P, A285L, A285V, A285I, A285M, F286L, F286I, F286W, F286Y, F286V, N288R, N288D, N288E, G289M, G290H, P291K, P291L, P291E, P291T, P291H, P291A, P291N, V292I, V292M, S293A, S293V, S293I, S293N, K294S, K294N, K294P, K294E, K294R, E295R, E295K, E295A, E295Q, E295D, Y296W, Y296R, Y296V, Y296F, Y296H, Y296L, P297A, P297T, P297Q, P297V, P297L, P297H, P297I, G298A, A299K, A299Q, A299R, S300A, S300R, V301I, V301A, K302A, K302R, K302D, K302Q, K302E, K303Q, K303W, K303R, K303E, I304L, I304G, I304Y, A305I, A305V, Q306H, Q306K, Q306E, Q306R, L307I, L307V, L307M, S308A, S308T, I310G, I310V, I310L, I310F, Q311R, Q311T, Q311K, Q311S, Q311D, Q311E, G312A, G312D, G312T, G312K, I313L, I313V, P314A, E315D, D316E, D316G, D316N, A317G, A317T, K318R, K318T, K318Y, V319F, V319L, V319M, V319I, V319Y, I320Y, I320L, V321L, V321P, V321C, V321I, S322A, S322T, S322G, S322V, C323E, C323L, C323I, C323G, C323P, C323V, C323K, T324I, T324V, T324L, R325K, R325E, R325D, R325T, G326D, Y327W, Y327F, Y327V, G328D, G328T, G328S, K329E, K329R, K329P, K329D, K329N, D330E, E331S, E331D, E331H, E331M, E331Y, P332R, P332T, L333M, L333V, L333F, L333I, A334V, A334S, A334M, A334C, K335R, K335Q, K335H, K337Q, K337E, L338F, L338V, L338I, L338M, F339C, F339M, V341I, L342M, L342V, L342I, A343C, A343T, A343V, A343S, A343P, F344I, F344V, F344L, F344M, F345Y, F345V, F345M, F345L, F345I, T346R, T346K, T346P, Y347F, Y347V, D348E, D348K, D348S, K349S, K349G, K349D, K349N, K349T, W350V, W350E, W350I, W350L, W350F, E351D, D352Q, D352H, D352E, I354L, I354V, I354C, H355R, H355A, H355D, H355Q, H355S, H355E, I356M, I356L, I356V, A357C, K358N, K358R, T359A, T359D, T359S, T359N, T359E, N360S, N360T, L361D, L361V, E362L, E362T, E362N, E362S, M363Y, M363E, E364Q, E364Y, E364F, E364H, I366Y, I366K, I366F, I366A, I366L, H368L, H368A, S369T, S369A, V370C, V370A, V371M, V371I, V371A, V371W, V371S, V371Y, I372V, I372M, H373W, H373Y, H373F, S374T, N375H, N375Q, N375K, N375T, N375R, T376N, T376S, T376D, P377D, P377I, P377V, P377E, E378K, E378D, E378A, H379N, H379K, H379V, H379R, I380A, I380M, I380V, E381M, E381W, E381K, E381R, E381T, E381H, A382K, A382R, A382E, A382Q, V383M, V383L, V383Y, V383F, V383I, A384S, A384G, E385Q, E385L, E385R, E385K, A386E, A386Q, A386K, A386R, I387M, I387V, I387L, P388R, P388H, P388D, P388Q, P388E, V389A, V389C, V389T, S390T, S390N, S390C, S390G, R391M, R391T, R391I, F392V, F392I, F392M, A393M, A393I, A393W, A393L, A393G, V394I, V394K, Q396R, Q396E, Q396S, Q396D, V397I, V397W, V397A, V397P, V397F, V397Y, G398S, G398C, G398P, G398T, G399A, G399S, T400I, T400S, T400F, T400V, T400H, N401G, N401H, N401D, N401S, N401A, L402N, L402A, L402F, L402M, G403S, A405I, A405F, A405V, A405G, M406Y, M406K, M406F, M406L, M406W, D407H, D407Q, D407Y, D407E, N408T, N408S, N408H, L410F, L410I, L410M, N411T, N411G, N411P, N411A, P412F, P412R, T413A, T414M, T414F, T414V, T415P, T415S, L416M, L416F, L416I, C418F, C418I, C418T, T420S, T420R, W421Y, W421F, N423K, N423R, N423G, N424S, N424R, S425I, S425G, S425W, S425A, I426V, I426T, I426L, S427G, S427T, S427A, E428D, E428H, N429E, F430I, F430Y, F430L, F430V, T431G, T431S, T431N, Y432A, Y432P, Y432F, Y432W, Y433T, Y433R, Y433K, Y433H, Y433M, Y433E, H434R, H434Q, H434D, H434N, L435V, L435F, L435W, L435Y, L435M, M436T, M436L, M436F, M436I, M436V, N437H, N437Q, N437Y, L438V, L438T, L438W, L438C, L438Y, L438I, T439Q, T439S, T439R, T439K, R440K, R440W, V441L, V441I, V441M, S442G, S442A, Y443H, Y443W, Y443R, Y443F, R444E, R444P, R444K, V445I, V445Y, V445F, V445M, V445L, P446E, P446K, D447E, D447N, M448W, M448K, M448P, Y449E, Y449R, Y449F, Y449H, I450M, I450C, I450F, I450V, I450L, P451W, T452S, T452D, T452K, D453E, D453P, D453H, E454A, E454D, E455K, E455Q, E455D, I456F, I456L, I456V, I456W, I456M, W457M, W457Y, W457F, A458H, A458G, E459D, or any combination thereof, in reference to SEQ ID NO: 3.

In some embodiments, an engineered ALDH described herein has one or more amino acid alterations comprising: E2Q, E2T, E2P, Q3S, Q3K, A4G, A4S, V5I, V5T, K6F, D7E, D7Q, L9V, K11E, K11D, K11Q, K11R, M12L, M12A, A15R, A15K, S16A, R17K, I18K, 118E, I18Q, Q20F, Q20H, Q21A, Q21R, E22K, E22Q, E22A, F23Y, A24E, A24K, T25A, T25E, Y26L, Y26M, Y26W, P27D, P27T, Q28W, T30D, T30K, T30Q, V31I, V31L, D32N, D32E, K33E, K33Q, K33A, K33R, A34V, V35C, V35M, R36K, T37K, T37A, T37R, V38L, V38I, G39A, K40W, A41L, A41V, I42L, I42V, I42M, I42C, Y43E, Y43L, D44K, D44Q, N45H, N45R, E47D, L48E, L48M, L48D, L49I, H51N, H51E, H51Q, M52L, V54L, D55E, D55R, K58R, N61D, N61E, N61R, Y62F, Y62V, Y62W, A63D, A63E, A63Q, D64H, K65R, I66V, A67E, V70M, V70R, N71D, N71E, N71Y, N71F, N71H, S73T, K74E, K74R, S75G, W77Y, W78E, W78F, W78Y, W78H, W78N, R79D, R79W, M80F, M80V, M80I, K81A, K81R, D82G, K83E, K84R, K84Q, R86V, R86K, I88M, I89V, I89L, K90S, K90D, K90H, K90E, K90R, R91E, R91Q, I92D, P93D, E94G, E94D, L95K, L95M, L95R, G96N, L97I, L97V, L97M, V98Y, V98I, V98M, V98T, E99R, E99T, E99Q, E99K, V100Y, V100I, V100M, V100L, A101Y, A101H, K102R, K102Q, I104F, I104M, I107C, I107V, A111T, A111S, V117I, V117T, V117L, N119T, N119S, V120A, V120T, V120M, M121L, M121V, M121I, Q122H, A124I, A124V, A124L, A124M, M125I, M125L, M125V, C126A, L128I, K129A, K129M, C130A, C130S, S133C, S133T, V136L, V136I, H144R, S145C, S145T, S145V, S146N, V147A, V147I, V147M, K148E, K148L, K148A, K148R, T149M, T149V, T149L, T149I, V150A, V150I, V150M, E151K, E151D, E151Q, V152M, V152I, I153M, I153C, I153F, I153V, N154Q, N154K, E155K, E155R, E155D, E155Q, A156I, L157A, L157F, L157I, A158E, A158V, A158K, A159D, A159K, L160V, L160A, M162F, M162Y, M162V, M162A, M162L, K164A, K164D, K164P, N165D, N165H, L166V, L166C, L166I, I167L, I167V, I167F, Q168T, Q168N, V169C, V169L, V169M, V169I, I170V, T171D, T171E, T171P, T171N, E172D, E172G, E172H, S174T, M175I, M175H, E176D, L177A, L177T, L177I, G180E, L181I, S183Q, S183A, S183H, S183K, A184G, A184S, L187M, L187V, L1871, L187F, L187K, I189V, I189L, C190A, P194A, P194T, P194R, P194K, L196V, V1971, K198R, A199V, A200C, Y201A, S202A, S202T, S202R, S203N, S203H, S203A, P206V, A207V, A207T, A207C, I208V, I208M, I208Y, Q212A, Q212P, N214S, V215P, V215N, V215C, V215A, V215S, Q216A, Q216P, V217M, V217A, V217C, L218I, L218V, V219I, V219M, V219L, R221P, R221D, D222T, D222S, A223C, D224N, L225V, Q227M, Q227E, Q227K, V228A, A229V, A230D, A230E, A230P, A230K, M231R, M231Q, M231I, M231K, V232M, I233L, K234F, K234A, K234W, K234M, K234Q, T237F, T237Y, T237C, T237M, F238R, F238Q, F238M, N240T, L243V, T245I, C246S, C246A, Q248N, N249Y, V250M, I251F, I251L, C252V, C252I, P253H, E254K, E254R, E254D, D255E, K256I, K256M, K256V, K256R, K257Y, K257A, E258D, E259R, E259A, E259Q, E259D, M260V, M260L, I261V, I261L, I261M, A262K, A263K, A263R, L264V, L264F, K265R, K265Q, K265E, K266E, H267R, Y270F, Y270L, Y270W, Y270M, Y270I, Y271W, Y271F, Y271K, Y271H, I272V, I272L, I272M, G273C, N274D, N274E, S275A, S275P, S275G, S275E, E276R, E276D, D277E, D277Q, A278E, A279S, A279E, K280T, L281I, L281V, L281M, R282K, R282Q, R282G, R282M, D283E, D283N, D283H, T284V, A285V, F286Y, F286V, F286I, N288E, G290H, P291L, P291A, P291T, P291H, P291N, P291K, S293A, S293I, S293N, K294R, E295D, E295R, E295Q, Y296L, Y296W, A299K, A299Q, A299R, S300A, V301A, K302A, K302E, K302Q, K303E, K303R, I304L, A305V, Q306E, Q306H, Q306K, Q306R, L307I, L307M, L307V, S308T, I310F, I310V, I310L, Q311D, Q311S, Q311R, Q311E, G312A, G312T, G312D, G312K, I313V, I313L, E315D, D316G, D316E, D316N, A317G, A317T, K318R, V319L, V319M, I320Y, I320L, V321I, V321L, V321C, S322G, S322V, S322A, S322T, C323I, C323V, C323K, C323P, T324I, T324V, T324L, R325E, R325T, G326D, Y327F, Y327W, Y327V, G328S, K329P, K329R, K329D, K329N, D330E, E331D, P332T, P332R, L333I, L333M, L333F, A334S, A334V, A334C, A334M, K335R, F339M, V341I, L342I, L342M, A343S, A343C, A343V, F344M, F344I, F344V, F344L, F345L, F345M, F345Y, F345V, T346P, Y347F, Y347V, D348E, D348K, D348S, K349G, K349T, K349N, K349S, K349D, W350E, W350I, W350V, W350F, W350L, E351D, I354L, I354C, H355D, H355A, H355Q, H355S, H355E, I356L, I356V, K358R, K358N, T359S, T359A, T359N, T359E, N360S, E362S, E362N, E362T, M363E, I366A, I366K, I366Y, I366L, V370C, V370A, V371S, V371A, I372V, I372M, H373F, H373Y, H373W, S374T, N375H, N375T, N375K, N375R, N375Q, T376D, P377E, P377I, P377V, E378K, E378A, E378D, H379R, H379N, H379V, I380V, E381W, E381H, E381M, E381R, E381K, E381T, A382K, V383I, V383M, A384G, A384S, E385R, E385K, A386Q, A386K, A386R, I387M, I387V, I387L, P388R, P388E, P388Q, P388H, P388D, V389T, V389A, V389C, S390G, S390T, S390C, F392M, F392V, F392I, A393G, V394I, Q396E, Q396D, Q396S, V397W, V397I, V397Y, T400S, T400I, T400H, N401D, N401G, N401S, N401H, N401A, L402M, A405V, A405G, M406W, M406F, M406L, D407H, D407Q, L410M, N411G, N411A, N411P, N411T, T414V, T420S, W421F, N423G, N423R, S425I, S425A, S425G, I426V, S427A, S427T, E428D, T431N, Y433T, Y433H, Y433K, Y433M, Y433R, L435M, L435V, M436V, M436F, M4361, N437Q, L438I, L438T, L438Y, L438C, T439S, R440W, R440K, V441M, V441I, V441L, Y443W, Y443F, Y443R, R444K, P446K, D447E, D447N, M448W, M448K, M448P, Y449R, Y449H, I450V, T452S, T452K, D453H, D453P, E454A, E454D, E455K, E455D, W457F, A458H, A458G, or E459D, in reference to SEQ ID NO: 3

In some embodiments, an engineered ALDH described herein has one or more amino acid alterations comprising: I42V, N119T or N119S, T149I, A158K, S174T, C246A, E259D, A263R, A299R, Y327V, D348S, V370A, F392I, N401A, or S427T, in reference to SEQ ID NO: 3. In some embodiments, an engineered ALDH described herein comprises an amino acid alteration comprising I42V in reference to SEQ ID NO: 3. In some embodiments, an engineered ALDH described herein comprises an amino acid alteration comprising N119T in reference to SEQ ID NO: 3. In some embodiments, an engineered ALDH described herein comprises an amino acid alteration comprising N119S in reference to SEQ ID NO: 3. In some embodiments, an engineered ALDH described herein comprises an amino acid alteration comprising T1491 in reference to SEQ ID NO: 3. In some embodiments, an engineered ALDH described herein comprises an amino acid alteration comprising A158K in reference to SEQ ID NO: 3. In some embodiments, an engineered ALDH described herein comprises an amino acid alteration comprising S174T in reference to SEQ ID NO: 3. In some embodiments, an engineered ALDH described herein comprises an amino acid alteration comprising C246A in reference to SEQ ID NO: 3. In some embodiments, an engineered ALDH described herein comprises an amino acid alteration comprising E259D in reference to SEQ ID NO: 3. In some embodiments, an engineered ALDH described herein comprises an amino acid alteration comprising A263R in reference to SEQ ID NO: 3. In some embodiments, an engineered ALDH described herein comprises an amino acid alteration comprising A299R in reference to SEQ ID NO: 3. In some embodiments, an engineered ALDH described herein comprises an amino acid alteration comprising Y327V in reference to SEQ ID NO: 3. In some embodiments, an engineered ALDH described herein comprises an amino acid alteration comprising D348S in reference to SEQ ID NO: 3. In some embodiments, an engineered ALDH described herein comprises an amino acid alteration comprising V370A in reference to SEQ ID NO: 3. In some embodiments, an engineered ALDH described herein comprises an amino acid alteration comprising F392I in reference to SEQ ID NO: 3. In some embodiments, an engineered ALDH described herein comprises an amino acid alteration comprising N401A in reference to SEQ ID NO: 3. In some embodiments, an engineered ALDH described herein comprises an amino acid alteration comprising S427T in reference to SEQ ID NO: 3.

In some embodiments, an engineered ALDH provided herein has an amino acid sequence that is a variant of SEQ ID NO: 3 that includes one or more alterations as described in TABLE 12, wherein the portion, other than the one or more alterations described in TABLE 12, of the engineered ALDH has at least 65%, 70%, 75%, 80%, 85%, 90%, 95%, 98% or 99% sequence identity, or is identical, to an amino acid sequence referenced as SEQ ID NO: 3. Accordingly, in some embodiments, an engineered ALDH provided herein has an amino acid sequence that includes one or more alterations as described in TABLE 12 and the portion, other than the alteration described in TABLE 12, of the engineered ALDH has at least 65% identical to SEQ ID NO: 3. In some embodiments, an engineered ALDH provided herein has an amino acid sequence that includes one or more alterations as described in TABLE 12 and the portion, other than the alteration described in TABLE 12, of the engineered ALDH has at least 70% identical to SEQ ID NO: 3. In some embodiments, an engineered ALDH provided herein has an amino acid sequence that includes one or more alterations as described in TABLE 12 and the portion, other than the alteration described in TABLE 12, of the engineered ALDH has at least 75% identical to SEQ ID NO: 3. In some embodiments, an engineered ALDH provided herein has an amino acid sequence that includes one or more alterations as described in TABLE 12 and the portion, other than the alteration described in TABLE 12, of the engineered ALDH has at least 80% identical to SEQ ID NO: 3. In some embodiments, an engineered ALDH provided herein has an amino acid sequence that includes one or more alterations as described in TABLE 12 and the portion, other than the alteration described in TABLE 12, of the engineered ALDH has at least 85% identical to SEQ ID NO: 3. In some embodiments, an engineered ALDH provided herein has an amino acid sequence that includes one or more alterations as described in TABLE 12 and the portion, other than the alteration described in TABLE 12, of the engineered ALDH has at least 90% identical to SEQ ID NO: 3. In some embodiments, an engineered ALDH provided herein has an amino acid sequence that includes one or more alterations as described in TABLE 12 and the portion, other than the alteration described in TABLE 12, of the engineered ALDH has at least 95% identical to SEQ ID NO: 3. In some embodiments, an engineered ALDH provided herein has an amino acid sequence that includes one or more alterations as described in TABLE 12 and the portion, other than the alteration described in TABLE 12, of the engineered ALDH has at least 98% identical to SEQ ID NO: 3. In some embodiments, an engineered ALDH provided herein has an amino acid sequence that includes one or more alterations as described in TABLE 12 and the portion, other than the alteration described in TABLE 12, of the engineered ALDH has at least 99% identical to SEQ ID NO: 3. In some embodiments, an engineered ALDH provided herein has an amino acid sequence that includes one or more alterations as described in TABLE 12 and the portion, other than the alteration described in TABLE 12, of the engineered ALDH is identical to SEQ ID NO: 3.

Sequence identity, homology or similarity refers to sequence similarity between two polypeptides or between two nucleic acid molecules. Identity can be determined by comparing a position in each sequence which may be aligned for purposes of comparison. When a position in the compared sequence is occupied by the same base or amino acid, then the molecules are identical at that position. A degree of homology between sequences is a function of the number of matching or homologous positions shared by the sequences. A polypeptide or polypeptide region (or a polynucleotide or polynucleotide region) has a certain percentage (for example, 65%, 70%, 75%, 80%, 85%, 90%, 95%, 98% or 99%) of “sequence identity” to another sequence means that, when aligned, that percentage of amino acids (or nucleotide bases) are the same in comparing the two sequences. The alignment of two sequences to determine their percent sequence identity can be done using software programs known in the art, such as, for example, those described in Ausubel et al., Current Protocols in Molecular Biology, John Wiley and Sons, Baltimore, MD (1999). Preferably, default parameters are used for the alignment. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases =non-redundant, GenBank+EMBL+DDBJ+PDB+GenBank CDS translations+SwissProtein+SPupdate+PIR. Details of these programs can be found at the National Center for Biotechnology Information (see also Altschul et al., “J. Mol. Biol. 215:403-410 (1990)).

Methods of generating and assaying the engineered ALDHs described herein are well known to one of skill in the art. Examples of such methods are described in the Examples provided herein. Any of a variety of methods can be used to generate an engineered ALDH disclosed herein. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). One non-limiting example of a method for preparing an engineered ALDH is to express recombinant nucleic acids encoding the engineered ALDH in a suitable microbial organism, such as a bacterial cell, a yeast cell, or other suitable cell, using methods well known in the art.

In some embodiments, an engineered ALDH provided herein is an isolated ALDH. An isolated engineered ALDH provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

### Recombinants

In some embodiments, the provided herein is a recombinant nucleic acid that has a nucleotide sequence encoding an engineered ACL described herein. Accordingly, in some embodiments, provided herein is a recombinant nucleic acid selected from (a) a nucleic acid molecule encoding an engineered ACL that is a variant of a wild-type ACL (SEQ ID NO: 1 or 2), such as an engineered ACL having one or more alterations at a position described in TABLE 8 or TABLE 9 and, in some embodiments, a combination of alternations described in TABLE 8 and 7 (b) a recombinant nucleic acid that hybridizes to an isolated nucleic acid of (a) under highly stringent hybridization conditions; and (c) a recombinant nucleic acid that is complementary to (a) or (b).

In some embodiments, provided herein is a recombinant nucleic acid encoding an engineered ACL that is a variant of a reference polypeptide, wherein the reference polypeptide has an amino acid sequence of SEQ ID NO: 1, and the engineered ACL has one or more alterations at a position described in TABLE 8 relative to SEQ ID NO: 1. It is understood that the positions identified the Tables (e.g., TABLE 8) correspond to codon positions in the encoding nucleic acid. An alteration in the nucleic acid would alter the corresponding codon at that position to encode an variant amino acid as described herein. In some embodiments, the recombinant nucleic acid encodes an engineered ACL that includes one or more amino acid alterations at a residue corresponding to position 4, 7, 8, 9, 10, 12, 13, 15, 16, 19, 20, 22, 30, 31, 35, 36, 39, 40, 43, 44, 47, 48, 49, 50, 51, 52, 53, 54, 55, 60, 61, 64, 66, 67, 69, 72, 73, 74, 76, 78, 80, 81, 82, 83, 86, 87, 88, 89, 90, 91, 94, 95, 96, 97, 98, 99, 100, 101, 102, 103, 104, 105, 106, 107, 109, 111, 112, 113, 114, 115, 116, 117, 119, 121, 126, 127, 128, 129, 130, 131, 136, 137, 138, 139, 140, 144, 149, 150, 152, 153, 159, 162, 163, 164, 165, 166, 167, 170, 172, 182, 189, 191, 194, 195, 196, 197, 198, 199, 200, 201, 202, 205, 208, 209, 211, 212, 213, 214, 215, 217, 220, 221, 222, 223, 224, 225, 226, 227, 228, 229, 230, 231, 232, 233, 234, 235, 238, 239, 240, 241, 242, 243, 244, 246, 247, 248, 249, 250, 251, 252, 253, 254, 255, 256, 257, 258, 259, 260, 261, 262, 264, 265, 266, 267, 268, 269, 270, 271, 272, 274, 275, 276, 277, 278, 279, 280, 281, 282, 286, 287, 288, 289, 290, 291, 294, 295, 296, 297, 298, 299, 300, 304, 306, 309, 310, 311, 314, 315, 316, 317, 318, 319, 320, 321, 323, 324, 326, 327, 328, 329, 330, 331, 334, 335, 336, 338, 339, 341, 342, 348, 351, 352, 353, 354, 355, 356, 357, 359, 360, 362, 363, 364, 365, 366, 367, 368, 369, 370, 371, 372, 373, 374, 375, 376, 377, 378, 379, 380, 381, 383, 384, 386, 387, 388, 389, 390, 391, 394, 398, 402, 406, 407, 410, 411, 412, 413, 414, 415, 416, 418, 419, 420, 424, 425, 426, 430, 431, 432, 433, 436, 442, 443, 444, 445, 446, 447, 455, 459, 461, 462, 464, 465, 469, 470, 471, 472, 474, 475, 476, 477, 478, 479, 480, 481, 484, 486, 489, 490, 491, 493, 494, 496, 503, 506, 507, 508, 509, 513, 518, 519, 521, 523, 524, 525, 527, 528, 529, 539, 540, 542, 546, 551, 553, 554, 555, 557, 559, 560, 562, 564, 565, 566, 583, more than one of the foregoing, or a combination thereof, in SEQ ID NO: 1. In some embodiments, the recombinant nucleic acid encodes an engineered ACL that includes one or more amino acid alterations at a residue corresponding to position 7, 8, 10, 12, 15, 16, 19, 52, 54, 64, 66, 67, 73, 74, 82, 87, 90, 94, 100, 111, 115, 117, 119, 121, 129, 131, 138, 139, 140, 144, 150, 165, 166, 172, 182, 191, 199, 209, 212, 217, 221, 223, 224, 227, 229, 230, 231, 232, 233, 234, 235, 238, 240, 243, 249, 253, 254, 256, 258, 259, 262, 268, 271, 275, 277, 281, 288, 290, 296, 297, 300, 304, 306, 316, 318, 320, 321, 323, 328, 330, 331, 336, 352, 353, 363, 364, 365, 366, 369, 372, 374, 376, 378, 388, 390, 394, 398, 406, 407, 416, 432, 443, 445, 455, 462, 469, 470, 471, 480, 481, 484, 486, 490, 491, 493, 494, 496, 508, 513, 518, 519, 521, 523, 525, 546, 554, 555, 560, 565, 583, more than one of the foregoing, or a combination thereof, in SEQ ID NO: 1. In some embodiments, the recombinant nucleic acid encodes an engineered ACL that includes one or more amino acid alterations at a residue corresponding to position 10, 199, 223, 229, 230, 231, 232, 233, 234, 235, 238, 243, 256, 258, 277, 288, 300, 318, 320, 321, 323, 328, 330, 364, 365, 369, 372, 374, 378, 462, 469, 480, 481, 484, 513, 523, 525, 554, 560, 583, more than one of the foregoing, or a combination thereof, in SEQ ID NO: 1. In some embodiments, the recombinant nucleic acid encodes an engineered ACL that includes one or more amino acid alterations at a residue corresponding to position 223, 232, 233, 234, 238, 258, 288, 318, 320, 321, 323, 364, 513, 523, more than one of the foregoing, or a combination thereof, or a combination thereof, in SEQ ID NO: 1.

In some embodiments, the recombinant nucleic acid encodes an engineered ACL having one or more alterations described in TABLE 8. Accordingly, in some embodiments, the recombinant nucleic acid encodes an engineered ACL having: M at a residue corresponding to position 4 in SEQ ID NO: 1; D, K, Q, S, T at a residue corresponding to position 7 in SEQ ID NO: 1; I, S at a residue corresponding to position 8 in SEQ ID NO: 1; A, K, S at a residue corresponding to position 9 in SEQ ID NO: 1; F, H, P, V, W, Y at a residue corresponding to position 10 in SEQ ID NO: 1; D, E, H, P at a residue corresponding to position 12 in SEQ ID NO: 1; C, M at a residue corresponding to position 13 in SEQ ID NO: 1; D, I, L, P at a residue corresponding to position 15 in SEQ ID NO: 1; E, H, N at a residue corresponding to position 16 in SEQ ID NO: 1; F, L, P, Q at a residue corresponding to position 19 in SEQ ID NO: 1; W at a residue corresponding to position 20 in SEQ ID NO: 1; S at a residue corresponding to position 22 in SEQ ID NO: 1; D at a residue corresponding to position 30 in SEQ ID NO: 1; A at a residue corresponding to position 31 in SEQ ID NO: 1; G, P at a residue corresponding to position 35 in SEQ ID NO: 1; H at a residue corresponding to position 36 in SEQ ID NO: 1; A at a residue corresponding to position 39 in SEQ ID NO: 1; C, F, L, M at a residue corresponding to position 40 in SEQ ID NO: 1; G at a residue corresponding to position 43 in SEQ ID NO: 1; D, H, S, T at a residue corresponding to position 44 in SEQ ID NO: 1; R, RTYTQKEL (SEQ ID NO: 15), RTYTQREL (SEQ ID NO: 16), RTYT (SEQ ID NO: 17) at a residue corresponding to position 47 in SEQ ID NO: 1; a deletion at a residue corresponding to position 48 in SEQ ID NO: 1; a deletion, Y at a residue corresponding to position 49 in SEQ ID NO: 1; a deletion, T at a residue corresponding to position 50 in SEQ ID NO: 1; a deletion, F, H, L, R, W, Y at a residue corresponding to position 51 in SEQ ID NO: 1; a deletion, K, R at a residue corresponding to position 52 in SEQ ID NO: 1; a deletion at a residue corresponding to position 53 in SEQ ID NO: 1; a deletion, L at a residue corresponding to position 54 in SEQ ID NO: 1; L at a residue corresponding to position 55 in SEQ ID NO: 1; R at a residue corresponding to position 60 in SEQ ID NO: 1; F, L at a residue corresponding to position 61 in SEQ ID NO: 1; A, G at a residue corresponding to position 64 in SEQ ID NO: 1; R at a residue corresponding to position 66 in SEQ ID NO: 1; A at a residue corresponding to position 67 in SEQ ID NO: 1; L at a residue corresponding to position 69 in SEQ ID NO: 1; G, T at a residue corresponding to position 72 in SEQ ID NO: 1; F, G, L, V at a residue corresponding to position 73 in SEQ ID NO: 1; K at a residue corresponding to position 74 in SEQ ID NO: 1; T at a residue corresponding to position 76 in SEQ ID NO: 1; V at a residue corresponding to position 78 in SEQ ID NO: 1; G at a residue corresponding to position 80 in SEQ ID NO: 1; PG at a residue corresponding to position 81 in SEQ ID NO: 1; A, E, G, N, R, T at a residue corresponding to position 82 in SEQ ID NO: 1; a deletion, K, P, R, S at a residue corresponding to position 83 in SEQ ID NO: 1; I at a residue corresponding to position 86 in SEQ ID NO: 1; I, V, VAV at a residue corresponding to position 87 in SEQ ID NO: 1; a deletion, A at a residue corresponding to position 88 in SEQ ID NO: 1; a deletion, I, L, V at a residue corresponding to position 89 in SEQ ID NO: 1; C, L, M, V, W at a residue corresponding to position 90 in SEQ ID NO: 1; A, L, M, S at a residue corresponding to position 91 in SEQ ID NO: 1; C, CIEAA (SEQ ID NO: 18), S, T, TIEAA (SEQ ID NO: 19) at a residue corresponding to position 94 in SEQ ID NO: 1; a deletion at a residue corresponding to position 95 in SEQ ID NO: 1; a deletion, E at a residue corresponding to position 96 in SEQ ID NO: 1; a deletion at a residue corresponding to position 97 in SEQ ID NO: 1; a deletion, A, L at a residue corresponding to position 98 in SEQ ID NO: 1; C, M at a residue corresponding to position 99 in SEQ ID NO: 1; S, M, L at a residue corresponding to position 100 in SEQ ID NO: 1; F, M at a residue corresponding to position 101 in SEQ ID NO: 1; H, F, L at a residue corresponding to position 102 in SEQ ID NO: 1; G at a residue corresponding to position 103 in SEQ ID NO: 1; V, L, T at a residue corresponding to position 104 in SEQ ID NO: 1; V, W, H, A, I at a residue corresponding to position 105 in SEQ ID NO: 1; S, Y, R, K at a residue corresponding to position 106 in SEQ ID NO: 1; A, C, V at a residue corresponding to position 107 in SEQ ID NO: 1; L, V, Q at a residue corresponding to position 109 in SEQ ID NO: 1; V, A, M, L, C, VTPAN (SEQ ID NO: 20) at a residue corresponding to position 111 in SEQ ID NO: 1; T, a deletion at a residue corresponding to position 112 in SEQ ID NO: 1; a deletion, L at a residue corresponding to position 113 in SEQ ID NO: 1; a deletion, A, L, V at a residue corresponding to position 114 in SEQ ID NO: 1; a deletion, N at a residue corresponding to position 115 in SEQ ID NO: 1; A, W, P at a residue corresponding to position 116 in SEQ ID NO: 1; S at a residue corresponding to position 117 in SEQ ID NO: 1; T at a residue corresponding to position 119 in SEQ ID NO: 1; R, D at a residue corresponding to position 121 in SEQ ID NO: 1; H at a residue corresponding to position 126 in SEQ ID NO: 1; M, L, MRHTG (SEQ ID NO: 21), MRITG (SEQ ID NO: 22) at a residue corresponding to position 127 in SEQ ID NO: 1; a deletion at a residue corresponding to position 128 in SEQ ID NO: 1; a deletion, I, H at a residue corresponding to position 129 in SEQ ID NO: 1; a deletion at a residue corresponding to position 130 in SEQ ID NO: 1; a deletion, G, D at a residue corresponding to position 131 in SEQ ID NO: 1; L at a residue corresponding to position 136 in SEQ ID NO: 1; T at a residue corresponding to position 137 in SEQ ID NO: 1; D, S at a residue corresponding to position 138 in SEQ ID NO: 1; E at a residue corresponding to position 139 in SEQ ID NO: 1; E, K, D at a residue corresponding to position 140 in SEQ ID NO: 1; T at a residue corresponding to position 144 in SEQ ID NO: 1; R at a residue corresponding to position 149 in SEQ ID NO: 1; I, L at a residue corresponding to position 150 in SEQ ID NO: 1; A, T, C, I at a residue corresponding to position 152 in SEQ ID NO: 1; G, S at a residue corresponding to position 153 in SEQ ID NO: 1; G at a residue corresponding to position 159 in SEQ ID NO: 1; S, H, R, SIDAF (SEQ ID NO: 23), RIESF (SEQ ID NO: 24), HFDSF (SEQ ID NO: 25), HFDAF (SEQ ID NO: 26), HIDSF (SEQ ID NO: 27) at a residue corresponding to position 162 in SEQ ID NO: 1; F, I, a deletion at a residue corresponding to position 163 in SEQ ID NO: 1; a deletion, D at a residue corresponding to position 164 in SEQ ID NO: 1; a deletion, S at a residue corresponding to position 165 in SEQ ID NO: 1; a deletion, F at a residue corresponding to position 166 in SEQ ID NO: 1; L at a residue corresponding to position 167 in SEQ ID NO: 1; G at a residue corresponding to position 170 in SEQ ID NO: 1; L, A, D at a residue corresponding to position 172 in SEQ ID NO: 1; Q at a residue corresponding to position 182 in SEQ ID NO: 1; E, G at a residue corresponding to position 189 in SEQ ID NO: 1; A at a residue corresponding to position 191 in SEQ ID NO: 1; D at a residue corresponding to position 194 in SEQ ID NO: 1; T, P, M at a residue corresponding to position 195 in SEQ ID NO: 1; T, I at a residue corresponding to position 196 in SEQ ID NO: 1; W, C at a residue corresponding to position 197 in SEQ ID NO: 1; Y, V, S, W, I, M, T at a residue corresponding to position 198 in SEQ ID NO: 1; M, L, W, P, F, C, A at a residue corresponding to position 199 in SEQ ID NO: 1; W at a residue corresponding to position 200 in SEQ ID NO: 1; F, T, N, P, Y, L at a residue corresponding to position 201 in SEQ ID NO: 1; T, H, A, Q, E, L, G at a residue corresponding to position 202 in SEQ ID NO: 1; T at a residue corresponding to position 205 in SEQ ID NO: 1; F at a residue corresponding to position 208 in SEQ ID NO: 1; P at a residue corresponding to position 209 in SEQ ID NO: 1; G at a residue corresponding to position 211 in SEQ ID NO: 1; A, C at a residue corresponding to position 212 in SEQ ID NO: 1; M, V, E at a residue corresponding to position 213 in SEQ ID NO: 1; V at a residue corresponding to position 214 in SEQ ID NO: 1; S at a residue corresponding to position 215 in SEQ ID NO: 1; Q, R, Y, H, K, G at a residue corresponding to position 217 in SEQ ID NO: 1; W, M, V at a residue corresponding to position 220 in SEQ ID NO: 1; T, A at a residue corresponding to position 221 in SEQ ID NO: 1; S, N at a residue corresponding to position 222 in SEQ ID NO: 1; A, N, I, L, H at a residue corresponding to position 223 in SEQ ID NO: 1; T, Q, H, I, N, M at a residue corresponding to position 224 in SEQ ID NO: 1; L, A, F, Q at a residue corresponding to position 225 in SEQ ID NO: 1; Y, C, T, W at a residue corresponding to position 226 in SEQ ID NO: 1; G, E, M, T, A, GKWSQD (SEQ ID NO: 28), GKWSQSY (SEQ ID NO: 29), GKWSQ (SEQ ID NO: 30), GKWSQDLK (SEQ ID NO: 31), GKWSQQLQ (SEQ ID NO: 32) at a residue corresponding to position 227 in SEQ ID NO: 1; a deletion at a residue corresponding to position 228 in SEQ ID NO: 1; a deletion, F, S, L, G, W, I, M, H at a residue corresponding to position 229 in SEQ ID NO: 1; a deletion, R, M, P, T, E, Q, A at a residue corresponding to position 230 in SEQ ID NO: 1; a deletion, Q, P, H, QDLQ (SEQ ID NO: 33) at a residue corresponding to position 231 in SEQ ID NO: 1; a deletion, W, A, Y, N, K, Q, D at a residue corresponding to position 232 in SEQ ID NO: 1; a deletion, W, R, M, Y, P at a residue corresponding to position 233 in SEQ ID NO: 1; a deletion, F, Y, T, K, R, Q, P, G, H, E at a residue corresponding to position 234 in SEQ ID NO: 1; H, S, R, K, M, Q, D, G at a residue corresponding to position 235 in SEQ ID NO: 1; P, N, H, T, D, DISAC (SEQ ID NO: 34), DVS at a residue corresponding to position 238 in SEQ ID NO: 1; V, F, R, a deletion at a residue corresponding to position 239 in SEQ ID NO: 1; a deletion, F, H, T, W, S, L, C, V at a residue corresponding to position 240 in SEQ ID NO: 1; a deletion, C, M, P, I at a residue corresponding to position 241 in SEQ ID NO: 1; a deletion, G, V, C at a residue corresponding to position 242 in SEQ ID NO: 1; L, C, M at a residue corresponding to position 243 in SEQ ID NO: 1; M, V at a residue corresponding to position 244 in SEQ ID NO: 1; W, F, L, Y at a residue corresponding to position 246 in SEQ ID NO: 1; F, C at a residue corresponding to position 247 in SEQ ID NO: 1; S, P at a residue corresponding to position 248 in SEQ ID NO: 1; A, V, M at a residue corresponding to position 249 in SEQ ID NO: 1; M, A, W at a residue corresponding to position 250 in SEQ ID NO: 1; A, H at a residue corresponding to position 251 in SEQ ID NO: 1; I, M, W, Q at a residue corresponding to position 252 in SEQ ID NO: 1; T, Q, N, I, H, V, M at a residue corresponding to position 253 in SEQ ID NO: 1; E, I, T, Y, H, V, L, P, A, M, F, S, G at a residue corresponding to position 254 in SEQ ID NO: 1; M, T, I, N, Y, C, ILLANV (SEQ ID NO: 35), ILLANVHY (SEQ ID NO: 36), ILLLNVHY (SEQ ID NO: 37), ILLLNV (SEQ ID NO: 38), ILLLNVHK (SEQ ID NO: 39), ILLHNVHK (SEQ ID NO: 40), ILLSNVHY (SEQ ID NO: 41) at a residue corresponding to position 255 in SEQ ID NO: 1; I, V, H, C, F, a deletion at a residue corresponding to position 256 in SEQ ID NO: 1; a deletion, L, T, N at a residue corresponding to position 257 in SEQ ID NO: 1; a deletion, S, L, P, M, H, T, A at a residue corresponding to position 258 in SEQ ID NO: 1; a deletion, C, A at a residue corresponding to position 259 in SEQ ID NO: 1; a deletion, F, L, M, V at a residue corresponding to position 260 in SEQ ID NO: 1; a deletion, Y, M, W, A, F, L, S at a residue corresponding to position 261 in SEQ ID NO: 1; a deletion, M, K, Y, R, G, C, L, V, S, N at a residue corresponding to position 262 in SEQ ID NO: 1; G, W, I, S, V at a residue corresponding to position 264 in SEQ ID NO: 1; R, C, H, K at a residue corresponding to position 265 in SEQ ID NO: 1; I, V, Q, C at a residue corresponding to position 266 in SEQ ID NO: 1; Y at a residue corresponding to position 267 in SEQ ID NO: 1; V, M, T, F, IMPR (SEQ ID NO: 42), IMNR (SEQ ID NO: 43) at a residue corresponding to position 268 in SEQ ID NO: 1; M, at a residue corresponding to position 269 in SEQ ID NO: 1; a deletion, P at a residue corresponding to position 270 in SEQ ID NO: 1; a deletion, R, Q at a residue corresponding to position 271 in SEQ ID NO: 1; W at a residue corresponding to position 272 in SEQ ID NO: 1; P, PEEMLRAIQ (SEQ ID NO: 44), PEEMLQAIQ (SEQ ID NO: 45) at a residue corresponding to position 274 in SEQ ID NO: 1; D, A, E, R, K, EEMLQAIQ (SEQ ID NO: 46), EEMLRAIQ (SEQ ID NO: 47), a deletion, KEMLQAIQ (SEQ ID NO: 48) at a residue corresponding to position 275 in SEQ ID NO: 1; a deletion at a residue corresponding to position 276 in SEQ ID NO: 1; a deletion, M, Y, L, F at a residue corresponding to position 277 in SEQ ID NO: 1; a deletion at a residue corresponding to position 278 in SEQ ID NO: 1; a deletion, Q, R, QAIQ (SEQ ID NO: 49), RAIQ (SEQ ID NO: 50) at a residue corresponding to position 279 in SEQ ID NO: 1; a deletion at a residue corresponding to position 280 in SEQ ID NO: 1; a deletion, V, M, L, I at a residue corresponding to position 281 in SEQ ID NO: 1; a deletion, Q at a residue corresponding to position 282 in SEQ ID NO: 1; P, C at a residue corresponding to position 286 in SEQ ID NO: 1; T, TVFFA (SEQ ID NO: 51), TSFFA (SEQ ID NO: 52), TFFFA (SEQ ID NO: 53), TMFFA (SEQ ID NO: 54), TVFHA (SEQ ID NO: 55) at a residue corresponding to position 287 in SEQ ID NO: 1; H, F, M, Y, R, I, V, T, C, a deletion at a residue corresponding to position 288 in SEQ ID NO: 1; a deletion, M, V, A, L, F, I at a residue corresponding to position 289 in SEQ ID NO: 1; a deletion, A, W, H, C, F, V, P, L at a residue corresponding to position 290 in SEQ ID NO: 1; a deletion, A, M, G, V at a residue corresponding to position 291 in SEQ ID NO: 1; A, T at a residue corresponding to position 294 in SEQ ID NO: 1; L, a deletion at a residue corresponding to position 295 in SEQ ID NO: 1; M, L at a residue corresponding to position 296 in SEQ ID NO: 1; M, Q, I, A, L, T at a residue corresponding to position 297 in SEQ ID NO: 1; M, A, F, R, IM at a residue corresponding to position 298 in SEQ ID NO: 1; M, F at a residue corresponding to position 299 in SEQ ID NO: 1; A at a residue corresponding to position 300 in SEQ ID NO: 1; M, R, E at a residue corresponding to position 304 in SEQ ID NO: 1; D at a residue corresponding to position 306 in SEQ ID NO: 1; L at a residue corresponding to position 309 in SEQ ID NO: 1; L, D at a residue corresponding to position 310 in SEQ ID NO: 1; L at a residue corresponding to position 311 in SEQ ID NO: 1; S, SHVRSV (SEQ ID NO: 56), SHVRTV (SEQ ID NO: 57) at a residue corresponding to position 314 in SEQ ID NO: 1; S, a deletion at a residue corresponding to position 315 in SEQ ID NO: 1; a deletion, V, L, I at a residue corresponding to position 316 in SEQ ID NO: 1; a deletion, H, N, Q, T at a residue corresponding to position 317 in SEQ ID NO: 1; a deletion, Y, M, T, S, C, Q, R, H, V, W, I, L at a residue corresponding to position 318 in SEQ ID NO: 1; a deletion, C, F, V, L, M at a residue corresponding to position 319 in SEQ ID NO: 1; F, I, V, Y, T, M, W at a residue corresponding to position 320 in SEQ ID NO: 1; C at a residue corresponding to position 321 in SEQ ID NO: 1; G at a residue corresponding to position 323 in SEQ ID NO: 1; S, M at a residue corresponding to position 324 in SEQ ID NO: 1; V, I, C at a residue corresponding to position 326 in SEQ ID NO: 1; S at a residue corresponding to position 327 in SEQ ID NO: 1; H, V, R, S, RELE (SEQ ID NO: 58) at a residue corresponding to position 328 in SEQ ID NO: 1; E, a deletion, ELE at a residue corresponding to position 329 in SEQ ID NO: 1; a deletion, L, T, I at a residue corresponding to position 330 in SEQ ID NO: 1; a deletion, M, E, C, L, I at a residue corresponding to position 331 in SEQ ID NO: 1; A, W, C, M, I at a residue corresponding to position 334 in SEQ ID NO: 1; Q, R at a residue corresponding to position 335 in SEQ ID NO: 1; K at a residue corresponding to position 336 in SEQ ID NO: 1; M at a residue corresponding to position 338 in SEQ ID NO: 1; S at a residue corresponding to position 339 in SEQ ID NO: 1; H, T at a residue corresponding to position 341 in SEQ ID NO: 1; L, F at a residue corresponding to position 342 in SEQ ID NO: 1; D, H, Y at a residue corresponding to position 348 in SEQ ID NO: 1; V, C, T, F, A, W at a residue corresponding to position 351 in SEQ ID NO: 1; E, K, D at a residue corresponding to position 352 in SEQ ID NO: 1; V, L, M at a residue corresponding to position 353 in SEQ ID NO: 1; C, F, L, Y, A, K, S, I at a residue corresponding to position 354 in SEQ ID NO: 1; E at a residue corresponding to position 355 in SEQ ID NO: 1; A at a residue corresponding to position 356 in SEQ ID NO: 1; W at a residue corresponding to position 357 in SEQ ID NO: 1; M at a residue corresponding to position 359 in SEQ ID NO: 1; S at a residue corresponding to position 360 in SEQ ID NO: 1; C, S at a residue corresponding to position 362 in SEQ ID NO: 1; A, T, G at a residue corresponding to position 363 in SEQ ID NO: 1; C, S at a residue corresponding to position 364 in SEQ ID NO: 1; L, I, M, C at a residue corresponding to position 365 in SEQ ID NO: 1; S, V, T at a residue corresponding to position 366 in SEQ ID NO: 1; A, C, S, H at a residue corresponding to position 367 in SEQ ID NO: 1; H at a residue corresponding to position 368 in SEQ ID NO: 1; H, Y, I, C, L, V, W, S, T, M at a residue corresponding to position 369 in SEQ ID NO: 1; Y, R, T, S, H, G, N, Q at a residue corresponding to position 370 in SEQ ID NO: 1; W at a residue corresponding to position 371 in SEQ ID NO: 1; K, W, Q, H, S, R, N at a residue corresponding to position 372 in SEQ ID NO: 1; S, H, P, Q, E at a residue corresponding to position 373 in SEQ ID NO: 1; D, T, A, R, S, P, W, E, Q, H at a residue corresponding to position 374 in SEQ ID NO: 1; P at a residue corresponding to position 375 in SEQ ID NO: 1; T, K, E, Q, H, D at a residue corresponding to position 376 in SEQ ID NO: 1; Y, R, N at a residue corresponding to position 377 in SEQ ID NO: 1; S, Y, P, V, A at a residue corresponding to position 378 in SEQ ID NO: 1; G at a residue corresponding to position 379 in SEQ ID NO: 1; T at a residue corresponding to position 380 in SEQ ID NO: 1; V, S, I at a residue corresponding to position 381 in SEQ ID NO: 1; Q, H, T, Y, W, P at a residue corresponding to position 383 in SEQ ID NO: 1; I, P, A, M at a residue corresponding to position 384 in SEQ ID NO: 1; S, A at a residue corresponding to position 386 in SEQ ID NO: 1; G, S, H at a residue corresponding to position 387 in SEQ ID NO: 1; T, M, V, L at a residue corresponding to position 388 in SEQ ID NO: 1; R, K, E at a residue corresponding to position 389 in SEQ ID NO: 1; C, L, A at a residue corresponding to position 390 in SEQ ID NO: 1; K at a residue corresponding to position 391 in SEQ ID NO: 1; D at a residue corresponding to position 394 in SEQ ID NO: 1; D, N at a residue corresponding to position 398 in SEQ ID NO: 1; V, L, I at a residue corresponding to position 402 in SEQ ID NO: 1; L, V at a residue corresponding to position 406 in SEQ ID NO: 1; D at a residue corresponding to position 407 in SEQ ID NO: 1; N, Q, NTENAPR (SEQ ID NO: 59), NTENAPD (SEQ ID NO: 60) at a residue corresponding to position 410 in SEQ ID NO: 1; T, TENAPD (SEQ ID NO: 61), TQNAPR (SEQ ID NO: 62), a deletion, TENAPR (SEQ ID NO: 63), TQNAPN (SEQ ID NO: 64) at a residue corresponding to position 411 in SEQ ID NO: 1; a deletion, Q at a residue corresponding to position 412 in SEQ ID NO: 1; a deletion, N, NNPN (SEQ ID NO: 65) at a residue corresponding to position 413 in SEQ ID NO: 1; a deletion, A, S, N at a residue corresponding to position 414 in SEQ ID NO: 1; a deletion, P at a residue corresponding to position 415 in SEQ ID NO: 1; a deletion, D, R, N, E at a residue corresponding to position 416 in SEQ ID NO: 1; P at a residue corresponding to position 418 in SEQ ID NO: 1; G at a residue corresponding to position 419 in SEQ ID NO: 1; E at a residue corresponding to position 420 in SEQ ID NO: 1; M, V at a residue corresponding to position 424 in SEQ ID NO: 1; V, L at a residue corresponding to position 425 in SEQ ID NO: 1; M, L at a residue corresponding to position 426 in SEQ ID NO: 1; T, N, S at a residue corresponding to position 430 in SEQ ID NO: 1; I, L at a residue corresponding to position 431 in SEQ ID NO: 1; T, F at a residue corresponding to position 432 in SEQ ID NO: 1; S, K at a residue corresponding to position 433 in SEQ ID NO: 1; W at a residue corresponding to position 436 in SEQ ID NO: 1; T at a residue corresponding to position 442 in SEQ ID NO: 1; R, K, RASFA (SEQ ID NO: 66) at a residue corresponding to position 443 in SEQ ID NO: 1; a deletion at a residue corresponding to position 444 in SEQ ID NO: 1; a deletion, M, T, S, A at a residue corresponding to position 445 in SEQ ID NO: 1; a deletion at a residue corresponding to position 446 in SEQ ID NO: 1; a deletion, A at a residue corresponding to position 447 in SEQ ID NO: 1; S, G at a residue corresponding to position 455 in SEQ ID NO: 1; G, E, D at a residue corresponding to position 459 in SEQ ID NO: 1; D, G at a residue corresponding to position 461 in SEQ ID NO: 1; R, G at a residue corresponding to position 462 in SEQ ID NO: 1; L, Y at a residue corresponding to position 464 in SEQ ID NO: 1; H at a residue corresponding to position 465 in SEQ ID NO: 1; M at a residue corresponding to position 469 in SEQ ID NO: 1; C, G at a residue corresponding to position 470 in SEQ ID NO: 1; Y, K, M, W, I, R at a residue corresponding to position 471 in SEQ ID NO: 1; I, F, C, Y, L, M at a residue corresponding to position 472 in SEQ ID NO: 1; E, EDGH (SEQ ID NO: 67), EDGY (SEQ ID NO: 68) at a residue corresponding to position 474 in SEQ ID NO: 1; D, a deletion at a residue corresponding to position 475 in SEQ ID NO: 1; a deletion at a residue corresponding to position 476 in SEQ ID NO: 1; a deletion, H, F, N, E, Y at a residue corresponding to position 477 in SEQ ID NO: 1; W, I, M at a residue corresponding to position 478 in SEQ ID NO: 1; T, H, Y, F, W at a residue corresponding to position 479 in SEQ ID NO: 1; V, L at a residue corresponding to position 480 in SEQ ID NO: 1; C, I, T at a residue corresponding to position 481 in SEQ ID NO: 1; L at a residue corresponding to position 484 in SEQ ID NO: 1; D at a residue corresponding to position 486 in SEQ ID NO: 1; I at a residue corresponding to position 489 in SEQ ID NO: 1; R, C, V at a residue corresponding to position 490 in SEQ ID NO: 1; G, R, N at a residue corresponding to position 491 in SEQ ID NO: 1; F, H, Y, L at a residue corresponding to position 493 in SEQ ID NO: 1; N at a residue corresponding to position 494 in SEQ ID NO: 1; F, S, Y at a residue corresponding to position 496 in SEQ ID NO: 1; L, V, I at a residue corresponding to position 503 in SEQ ID NO: 1; R, RHPD (SEQ ID NO: 69) at a residue corresponding to position 506 in SEQ ID NO: 1; a deletion at a residue corresponding to position 507 in SEQ ID NO: 1; a deletion, P at a residue corresponding to position 508 in SEQ ID NO: 1; a deletion, D at a residue corresponding to position 509 in SEQ ID NO: 1; A, V, C at a residue corresponding to position 513 in SEQ ID NO: 1; V at a residue corresponding to position 518 in SEQ ID NO: 1; P at a residue corresponding to position 519 in SEQ ID NO: 1; E, P at a residue corresponding to position 521 in SEQ ID NO: 1; Q, A, R, M, Y, D at a residue corresponding to position 523 in SEQ ID NO: 1; S, A at a residue corresponding to position 524 in SEQ ID NO: 1; S, T, D, G, E at a residue corresponding to position 525 in SEQ ID NO: 1; P, T, V at a residue corresponding to position 527 in SEQ ID NO: 1; A, V at a residue corresponding to position 528 in SEQ ID NO: 1; V, A at a residue corresponding to position 529 in SEQ ID NO: 1; L, V at a residue corresponding to position 539 in SEQ ID NO: 1; T at a residue corresponding to position 540 in SEQ ID NO: 1; E, D at a residue corresponding to position 542 in SEQ ID NO: 1; A, K at a residue corresponding to position 546 in SEQ ID NO: 1; K, R at a residue corresponding to position 551 in SEQ ID NO: 1; S at a residue corresponding to position 553 in SEQ ID NO: 1; S, P, H at a residue corresponding to position 554 in SEQ ID NO: 1; F at a residue corresponding to position 555 in SEQ ID NO: 1; K at a residue corresponding to position 557 in SEQ ID NO: 1; P at a residue corresponding to position 559 in SEQ ID NO: 1; G at a residue corresponding to position 560 in SEQ ID NO: 1; V, a deletion at a residue corresponding to position 562 in SEQ ID NO: 1; F at a residue corresponding to position 564 in SEQ ID NO: 1; V, F, FV at a residue corresponding to position 565 in SEQ ID NO: 1; E, VE at a residue corresponding to position 566 in SEQ ID NO: 1; Q, E at a residue corresponding to position 583 in SEQ ID NO: 1; more than one of the foregoing; or any combination thereof. It is understood that when alterations are listed herein for a certain position, the alteration can be in the alternative or in the conjunctive depending on the context. For example, the deletion, Y, M, T, S, C, Q, R, H, V, W, I, L at a residue corresponding to position 318 in SEQ ID NO: 1, means that alterations at a residue corresponding to position 318 in SEQ ID NO: 1 can comprise a deletion or Y or M or T or S or C or Q or R or H or V or W or I or L. In another example, the V, F, FV alterations at a residue corresponding to position 565 in SEQ ID NO: 1, means that alterations at a residue corresponding to position 565 in SEQ ID NO: 1 can comprise V or F or FV, wherein FV is F and V.

In some embodiments, the recombinant nucleic acid encodes an engineered ACL having: K, S at a residue corresponding to position 7 in SEQ ID NO: 1; S at a residue corresponding to position 8 in SEQ ID NO: 1; W, Y, F at a residue corresponding to position 10 in SEQ ID NO: 1; E, P, D at a residue corresponding to position 12 in SEQ ID NO: 1; I at a residue corresponding to position 15 in SEQ ID NO: 1; E at a residue corresponding to position 16 in SEQ ID NO: 1; F at a residue corresponding to position 19 in SEQ ID NO: 1; R, K at a residue corresponding to position 52 in SEQ ID NO: 1; L at a residue corresponding to position 54 in SEQ ID NO: 1; A at a residue corresponding to position 64 in SEQ ID NO: 1; R at a residue corresponding to position 66 in SEQ ID NO: 1; A at a residue corresponding to position 67 in SEQ ID NO: 1; L at a residue corresponding to position 73 in SEQ ID NO: 1; K at a residue corresponding to position 74 in SEQ ID NO: 1; T at a residue corresponding to position 82 in SEQ ID NO: 1; I at a residue corresponding to position 87 in SEQ ID NO: 1; L, M at a residue corresponding to position 90 in SEQ ID NO: 1; T at a residue corresponding to position 94 in SEQ ID NO: 1; L at a residue corresponding to position 100 in SEQ ID NO: 1; V at a residue corresponding to position 111 in SEQ ID NO: 1; N at a residue corresponding to position 115 in SEQ ID NO: 1; S at a residue corresponding to position 117 in SEQ ID NO: 1; T at a residue corresponding to position 119 in SEQ ID NO: 1; D at a residue corresponding to position 121 in SEQ ID NO: 1; I at a residue corresponding to position 129 in SEQ ID NO: 1; G at a residue corresponding to position 131 in SEQ ID NO: 1; D, S at a residue corresponding to position 138 in SEQ ID NO: 1; E at a residue corresponding to position 139 in SEQ ID NO: 1; K, E, D at a residue corresponding to position 140 in SEQ ID NO: 1; T at a residue corresponding to position 144 in SEQ ID NO: 1; L, I at a residue corresponding to position 150 in SEQ ID NO: 1; S at a residue corresponding to position 165 in SEQ ID NO: 1; F at a residue corresponding to position 166 in SEQ ID NO: 1; L, A, D at a residue corresponding to position 172 in SEQ ID NO: 1; Q at a residue corresponding to position 182 in SEQ ID NO: 1; A at a residue corresponding to position 191 in SEQ ID NO: 1; A at a residue corresponding to position 199 in SEQ ID NO: 1; P at a residue corresponding to position 209 in SEQ ID NO: 1; C at a residue corresponding to position 212 in SEQ ID NO: 1; R at a residue corresponding to position 217 in SEQ ID NO: 1; T, A at a residue corresponding to position 221 in SEQ ID NO: 1; N, I, A, L, H at a residue corresponding to position 223 in SEQ ID NO: 1; Q at a residue corresponding to position 224 in SEQ ID NO: 1; E, T, A at a residue corresponding to position 227 in SEQ ID NO: 1; I, L, M at a residue corresponding to position 229 in SEQ ID NO: 1; Q, A at a residue corresponding to position 230 in SEQ ID NO: 1; H, Q, P at a residue corresponding to position 231 in SEQ ID NO: 1; Y, K, Q, N at a residue corresponding to position 232 in SEQ ID NO: 1; W, Y at a residue corresponding to position 233 in SEQ ID NO: 1; Y, F, H at a residue corresponding to position 234 in SEQ ID NO: 1; S at a residue corresponding to position 235 in SEQ ID NO: 1; D, H, T at a residue corresponding to position 238 in SEQ ID NO: 1; T, H, C, S at a residue corresponding to position 240 in SEQ ID NO: 1; C, M at a residue corresponding to position 243 in SEQ ID NO: 1; M, V at a residue corresponding to position 249 in SEQ ID NO: 1; H, M, Q at a residue corresponding to position 253 in SEQ ID NO: 1; M at a residue corresponding to position 254 in SEQ ID NO: 1; I, V at a residue corresponding to position 256 in SEQ ID NO: 1; T, H, A, L, M at a residue corresponding to position 258 in SEQ ID NO: 1; A at a residue corresponding to position 259 in SEQ ID NO: 1; K, C, N at a residue corresponding to position 262 in SEQ ID NO: 1; F at a residue corresponding to position 268 in SEQ ID NO: 1; R, Q at a residue corresponding to position 271 in SEQ ID NO: 1; E at a residue corresponding to position 275 in SEQ ID NO: 1; L, M at a residue corresponding to position 277 in SEQ ID NO: 1; V at a residue corresponding to position 281 in SEQ ID NO: 1; V, I, F, T, H, Y at a residue corresponding to position 288 in SEQ ID NO: 1; V, A at a residue corresponding to position 290 in SEQ ID NO: 1; L, M at a residue corresponding to position 296 in SEQ ID NO: 1; I at a residue corresponding to position 297 in SEQ ID NO: 1; A at a residue corresponding to position 300 in SEQ ID NO: 1; E at a residue corresponding to position 304 in SEQ ID NO: 1; D at a residue corresponding to position 306 in SEQ ID NO: 1; L at a residue corresponding to position 316 in SEQ ID NO: 1; S, R, H, W, Y, M, T, C, Q, V, I, L at a residue corresponding to position 318 in SEQ ID NO: 1; M, F, T, Y, W at a residue corresponding to position 320 in SEQ ID NO: 1; C at a residue corresponding to position 321 in SEQ ID NO: 1; G at a residue corresponding to position 323 in SEQ ID NO: 1; V, H, R at a residue corresponding to position 328 in SEQ ID NO: 1; I, L at a residue corresponding to position 330 in SEQ ID NO: 1; I at a residue corresponding to position 331 in SEQ ID NO: 1; K at a residue corresponding to position 336 in SEQ ID NO: 1; K at a residue corresponding to position 352 in SEQ ID NO: 1; V, L at a residue corresponding to position 353 in SEQ ID NO: 1; G at a residue corresponding to position 363 in SEQ ID NO: 1; S at a residue corresponding to position 364 in SEQ ID NO: 1; C, L, I, M at a residue corresponding to position 365 in SEQ ID NO: 1; V at a residue corresponding to position 366 in SEQ ID NO: 1; H, S, T, M at a residue corresponding to position 369 in SEQ ID NO: 1; S, K, H, N at a residue corresponding to position 372 in SEQ ID NO: 1; Q, R, T, P, H, S, A, D, E at a residue corresponding to position 374 in SEQ ID NO: 1; T at a residue corresponding to position 376 in SEQ ID NO: 1; A, P, S, V at a residue corresponding to position 378 in SEQ ID NO: 1; T at a residue corresponding to position 388 in SEQ ID NO: 1; C, A at a residue corresponding to position 390 in SEQ ID NO: 1; D at a residue corresponding to position 394 in SEQ ID NO: 1; N at a residue corresponding to position 398 in SEQ ID NO: 1; L at a residue corresponding to position 406 in SEQ ID NO: 1; D at a residue corresponding to position 407 in SEQ ID NO: 1; N, E at a residue corresponding to position 416 in SEQ ID NO: 1; F at a residue corresponding to position 432 in SEQ ID NO: 1; R, K at a residue corresponding to position 443 in SEQ ID NO: 1; A at a residue corresponding to position 445 in SEQ ID NO: 1; S, G at a residue corresponding to position 455 in SEQ ID NO: 1; R at a residue corresponding to position 462 in SEQ ID NO: 1; M at a residue corresponding to position 469 in SEQ ID NO: 1; G at a residue corresponding to position 470 in SEQ ID NO: 1; R, M, W at a residue corresponding to position 471 in SEQ ID NO: 1; V at a residue corresponding to position 480 in SEQ ID NO: 1; I, C at a residue corresponding to position 481 in SEQ ID NO: 1; L at a residue corresponding to position 484 in SEQ ID NO: 1; D at a residue corresponding to position 486 in SEQ ID NO: 1; C at a residue corresponding to position 490 in SEQ ID NO: 1; R, G at a residue corresponding to position 491 in SEQ ID NO: 1; L, H, Y at a residue corresponding to position 493 in SEQ ID NO: 1; N at a residue corresponding to position 494 in SEQ ID NO: 1; Y at a residue corresponding to position 496 in SEQ ID NO: 1; P at a residue corresponding to position 508 in SEQ ID NO: 1; A, V, C at a residue corresponding to position 513 in SEQ ID NO: 1; V at a residue corresponding to position 518 in SEQ ID NO: 1; P at a residue corresponding to position 519 in SEQ ID NO: 1; E, P at a residue corresponding to position 521 in SEQ ID NO: 1; R, Q, A, Y, M, D at a residue corresponding to position 523 in SEQ ID NO: 1; D, S, T, G, E at a residue corresponding to position 525 in SEQ ID NO: 1; K at a residue corresponding to position 546 in SEQ ID NO: 1; H, S, P at a residue corresponding to position 554 in SEQ ID NO: 1; F at a residue corresponding to position 555 in SEQ ID NO: 1; G at a residue corresponding to position 560 in SEQ ID NO: 1; V at a residue corresponding to position 565 in SEQ ID NO: 1; Q, E at a residue corresponding to position 583 in SEQ ID NO: 1; or any combination thereof. It is understood that when alterations are listed herein for a certain position, the alteration can be in the alternative or in the conjunctive depending on the context. For example, the S, R, H, W, Y, M, T, C, Q, V, I, L at a residue corresponding to position 318 in SEQ ID NO: 1, means that alterations at a residue corresponding to position 318 in SEQ ID NO: 1 can comprise S or R or H or W or Y or M or T or C or Q or V or I or L.

In some embodiments, the recombinant nucleic acid encodes an engineered ACL having: F at a residue corresponding to position 10 in SEQ ID NO: 1; A at a residue corresponding to position 199 in SEQ ID NO: 1; L, H, A at a residue corresponding to position 223 in SEQ ID NO: 1; M at a residue corresponding to position 229 in SEQ ID NO: 1; Q, A at a residue corresponding to position 230 in SEQ ID NO: 1; P at a residue corresponding to position 231 in SEQ ID NO: 1; N at a residue corresponding to position 232 in SEQ ID NO: 1; W, Y at a residue corresponding to position 233 in SEQ ID NO: 1; H at a residue corresponding to position 234 in SEQ ID NO: 1; S at a residue corresponding to position 235 in SEQ ID NO: 1; T at a residue corresponding to position 238 in SEQ ID NO: 1; C, M at a residue corresponding to position 243 in SEQ ID NO: 1; I, V at a residue corresponding to position 256 in SEQ ID NO: 1; L, M at a residue corresponding to position 258 in SEQ ID NO: 1; M at a residue corresponding to position 277 in SEQ ID NO: 1; Y, H at a residue corresponding to position 288 in SEQ ID NO: 1; A at a residue corresponding to position 300 in SEQ ID NO: 1; V, T, C, I, L, Q, M, Y at a residue corresponding to position 318 in SEQ ID NO: 1; W, Y at a residue corresponding to position 320 in SEQ ID NO: 1; C at a residue corresponding to position 321 in SEQ ID NO: 1; G at a residue corresponding to position 323 in SEQ ID NO: 1; R at a residue corresponding to position 328 in SEQ ID NO: 1; L at a residue corresponding to position 330 in SEQ ID NO: 1; S at a residue corresponding to position 364 in SEQ ID NO: 1; I, M at a residue corresponding to position 365 in SEQ ID NO: 1; M at a residue corresponding to position 369 in SEQ ID NO: 1; N at a residue corresponding to position 372 in SEQ ID NO: 1; E at a residue corresponding to position 374 in SEQ ID NO: 1; V at a residue corresponding to position 378 in SEQ ID NO: 1; R at a residue corresponding to position 462 in SEQ ID NO: 1; M at a residue corresponding to position 469 in SEQ ID NO: 1; V at a residue corresponding to position 480 in SEQ ID NO: 1; C at a residue corresponding to position 481 in SEQ ID NO: 1; L at a residue corresponding to position 484 in SEQ ID NO: 1; C, A, V at a residue corresponding to position 513 in SEQ ID NO: 1; D at a residue corresponding to position 523 in SEQ ID NO: 1; G, E, T at a residue corresponding to position 525 in SEQ ID NO: 1; S, P at a residue corresponding to position 554 in SEQ ID NO: 1; G at a residue corresponding to position 560 in SEQ ID NO: 1; E at a residue corresponding to position 583 in SEQ ID NO: 1; or any combination thereof. It is understood that when alterations are listed herein for a certain position, the alteration can be in the alternative or in the conjunctive depending on the context. For example, the V, T, C, I, L, Q, M, Y at a residue corresponding to position 318 in SEQ ID NO: 1, means that alterations at a residue corresponding to position 318 in SEQ ID NO: 1 can comprise V or T or C or I or L or Q or M or Y.

In some embodiments, the recombinant nucleic acid encodes an engineered ACL having: L, H at a residue corresponding to position 223 in SEQ ID NO: 1; N at a residue corresponding to position 232 in SEQ ID NO: 1; W, Y at a residue corresponding to position 233 in SEQ ID NO: 1; H at a residue corresponding to position 234 in SEQ ID NO: 1; T at a residue corresponding to position 238 in SEQ ID NO: 1; L at a residue corresponding to position 258 in SEQ ID NO: 1; Y, H at a residue corresponding to position 288 in SEQ ID NO: 1; V, T, C, I, L at a residue corresponding to position 318 in SEQ ID NO: 1; W, Y at a residue corresponding to position 320 in SEQ ID NO: 1; C at a residue corresponding to position 321 in SEQ ID NO: 1; G at a residue corresponding to position 323 in SEQ ID NO: 1; S at a residue corresponding to position 364 in SEQ ID NO: 1; C, A at a residue corresponding to position 513 in SEQ ID NO: 1; D at a residue corresponding to position 523 in SEQ ID NO: 1; or any combination thereof. It is understood that when alterations are listed herein for a certain position, the alteration can be in the alternative or in the conjunctive depending on the context. For example, the V, T, C, I, L at a residue corresponding to position 318 in SEQ ID NO: 1, means that alterations at a residue corresponding to position 318 in SEQ ID NO: 1 can comprise V or T or C or I or L.

In some embodiments, the recombinant nucleic acid encodes an engineered ACL having one or more amino acid alterations comprising: I4M, N7D, N7K, N7Q, N7S, N7T, A8I, A8S, T9A, T9K, T9S, I10F, I10H, I10P, I10V, I10W, I10Y, N12D, N12E, N12H, N12P, I13C, I13M, V15D, V15I, V15L, V15P, D16E, D16H, D16N, Y19F, Y19L, Y19P, Y19Q, D20W, K22S, G30D, G31A, E35G, E35P, T36H, S39A, V40C, V40F, V40L, V40M, A43G, K44D, K44H, K44S, K44T, Q47R, Q47RTYTQKEL (“RTYTQKEL” disclosed as SEQ ID NO: 15), Q47RTYTQREL (“RTYTQREL” disclosed as SEQ ID NO: 16), Q47RTYT (“RTYT” disclosed as SEQ ID NO: 17), d48, d49, L49Y, d50, S50T, d51, Q51F, Q51H, Q51L, Q51R, Q51W, Q51Y, d52, A52K, A52R, d53, d54, M54L, W55L, K60R, Y61F, Y61L, L64A, L64G, S66R, S67A, Y69L, C72G, C72T, R73F, R73G, R73L, R73V, H74K, D76T, E78V, D80G, P81PG, S82A, S82E, S82G, S82N, S82R, S82T, d83, M83K, M83P, M83R, M83S, V86I, L87I, L87V, L87VAV, d88, I88A, d89, T89I, T89L, T89V, F90C, F90L, F90M, F90V, F90W, F91A, F91L, F91M, F91S, V94C, V94CIEAA (“CIEAA” disclosed as SEQ IDNO: 18), V94S, V94T, V94TIEAA (“TIEAA” disclosed as SEQ ID NO: 19), d95, d96, L96E, d97, d98, P98A, P98L, V99C, V99M, V100S, V100M, V100L, H101F, H101M, W102H, W102F, W102L, A103G, A104V, A104L, A104T, L105V, L105W, L105H, L105A, L105I, D106S, D106Y, D106R, D106K, L107A, L107C, L107V, A109L, A109V, A109Q, I111V, I111A, I111M, 111L, I111C, I111VTPAN (“VTPAN” disclosed as SEQ ID NO: 20), S112T, d112, d113, P113L, d114, G114A, G114L, G114V, d115, S115N, T116A, T116W, T116P, G117S, S119T, Q121R, Q121D, Q126H, F127M, F127L, F127MRHTG (“MRHTG” disclosed as SEQ ID NO: 21), F127MRITG (“MRITG” disclosed as SEQ ID NO: 22), d128, d129, V129I, V129H, d130, d131, T131G, T131D, V136L, Y137T, A138D, A138S, K139E, A140E, A140K, A140D, V144T, T149R, K150I, K150L, Y152A, Y152T, Y152C, Y152I, N153G, N153S, A159G, E162S, E162H, E162R, E162SIDAF (“SIDAF” disclosed as SEQ ID NO: 23), E162RIESF (“RIESF” disclosed as SEQ ID NO: 24), E162HFDSF (“HFDSF” disclosed as SEQ ID NO: 25), E162HFDAF (“HFDAF” disclosed as SEQ ID NO: 26), E162HIDSF (“HIDSF” disclosed as SEQ ID NO: 27), L163F, L163I, d163, d164, E164D, d165, A165S, d166, L166F, D167L, A170G, M172L, M172A, M172D, R182Q, H189E, H189G, S191A, R194D, I195T, I195P, I195M, A196T, A196I, Y197W, Y197C, L198Y, L198V, L198S, L198W, L198I, L198M, L198T, G199M, G199L, G199W, G199P, G199F, G199C, G199A, M200W, S201F, S201T, S201N, S201P, S201Y, S201L, S202T, S202H, S202A, S202Q, S202E, S202L, S202G, S205T, V208F, S209P, A211G, V212A, V212C, R213M, R213V, R213E, L214V, T215S, S217Q, S217R, S217Y, S217H, S217K, S217G, T220W, T220M, T220V, S221T, S221A, C222S, C222N, S223A, S223N, S223I, S223L, S223H, E224T, E224Q, E224H, E224I, E224N, E224M, I225L, I225A, I225F, I225Q, S226Y, S226C, S226T, S226W, N227G, N227E, N227M, N227T, N227A, N227GKWSQD (“GKWSQD” disclosed as SEQ ID NO: 28), N227GKWSQSY (“GKWSQSY” disclosed as SEQ ID NO: 29), N227GKWSQ (“GKWSQ” disclosed as SEQ ID NO: 30), N227GKWSQDLK (“GKWSQDLK” disclosed as SEQ ID NO: 31), N227GKWSQQLQ (“GKWSQQLQ” disclosed as SEQ ID NO: 32), d228, d229, A229F, A229S, A229L, A229G, A229W, A229I, A229M, A229H, d230, S230R, S230M, S230P, S230T, S230E, S230Q, S230A, d231, E231Q, E231P, E231H, E231QDLQ (“QDLQ” disclosed as SEQ ID NO: 33), d232, S232W, S232A, S232Y, S232N, S232K, S232Q, S232D, d233, L233W, L233R, L233M, L233Y, L233P, d234, A234F, A234Y, A234T, A234K, A234R, A234Q, A234P, A234G, A234H, A234E, T235H, T235S, T235R, T235K, T235M, T235Q, T235D, T235G, Q238P, Q238N, Q238H, Q238T, Q238D, Q238DISAC (“DISAC” disclosed as SEQ ID NO: 34), Q238DVS, I239V, I239F, I239R, d239, d240, A240F, A240H, A240T, A240W, A240S, A240L, A240C, A240V, d241, A241C, A241M, A241P, A241I, d242, A242G, A242V, A242C, I243L, I243C, I243M, I244M, I244V, V246W, V246F, V246L, V246Y, S247F, S247C, H248S, H248P, L249A, L249V, L249M, F250M, F250A, F250W, G251A, G251H, L252I, L252M, L252W, L252Q, S253T, S253Q, S253N, S253I, S253H, S253V, S253M, K254E, K254I, K254T, K254Y, K254H, K254V, K254L, K254P, K254A, K254M, K254F, K254S, K254G, F255M, F255T, F255I, F255N, F255Y, F255C, F255ILLANV (“ILLANV” disclosed as SEQ ID NO: 35), F255ILLANVHY (“ILLANVHY” disclosed as SEQ ID NO: 36), F255ILLLNVHY (“ILLLNVHY” disclosed as SEQ ID NO: 37), F255ILLLNV (“ILLLNV” disclosed as SEQ ID NO: 38), F255ILLLNVHK (“ILLLNVHK” disclosed as SEQ ID NO: 39), F255ILLHNVHK (“ILLHNVHK” disclosed as SEQ ID NO: 40), F255ILLSNVHY (“ILLSNVHY” disclosed as SEQ ID NO: 41), L256I, L256V, L256H, L256C, L256F, d256, d257, I257L, I257T, I257N, d258, G258S, G258L, G258P, G258M, G258H, G258T, G258A, d259, N259C, N259A, d260, P260F, P260L, P260M, P260V, d261, H261Y, H261M, H261W, H261A, H261F, H261L, H261S, d262, A262M, A262K, A262Y, A262R, A262G, A262C, A262L, A262V, A262S, A262N, A264G, A264W, A264I, A264S, A264V, T265R, T265C, T265H, T265K, T266I, T266V, T266Q, T266C, V267Y, Y268V, Y268M, Y268T, Y268F, Y268IMPR (“IMPR” disclosed as SEQ ID NO: 42), Y268IMNR (“IMNR” disclosed as SEQ ID NO: 43), H269M, d269, d270, N270P, d271, G271R, G271Q, F272W, L274P, L274PEEMLRAIQ (“PEEMLRAIQ” disclosed as SEQ ID NO: 44), L274PEEMLQAIQ (“PEEMLQAIQ” disclosed as SEQ ID NO: 45), 1275D, I275A, 1275E, I275R, I275K, I275EEMLQAIQ (“EEMLQAIQ” disclosed as SEQ ID NO: 46), I275EEMLRAIQ (“EEMLRAIQ” disclosed as SEQ ID NO: 47), d275-282, I275KEMLQAIQ (“KEMLQAIQ” disclosed as SEQ ID NO: 48), d276, d277, V277M, V277Y, V277L, V277F, d278, d279, E279Q, E279R, E279QAIQ (“QAIQ” disclosed as SEQ ID NO: 49), E279RAIQ (“RAIQ” disclosed as SEQ ID NO: 50), d280, d281, Q281V, Q281M, Q281L, Q281I, d282, K282Q, V286P, V286C, N287T, N287TVFFA (“TVFFA” disclosed as SEQ ID NO: 51), N287TSFFA (“TSFFA” disclosed as SEQ ID NO: 52), N287TFFFA (“TFFFA” disclosed as SEQ ID NO: 53), N287TMFFA (“TMFFA” disclosed as SEQ ID NO: 54), N287TVFHA (“TVFHA” disclosed as SEQ ID NO: 55), S288H, S288F, S288M, S288Y, S288R, S288I, S288V, S288T, S288C, d288, d289, W289M, W289V, W289A, W289L, W289F, W289I, d290, T290A, T290W, T290H, T290C, T290F, T290V, T290P, T290L, d291, L291A, L291M, L291G, L291V, P294A, P294T, I295L, d295, I296M, I296L, V297M, V297Q, V297I, V297A, V297L, V297T, L298M, L298A, L298F, L298R, L298IM, L299M, L299F, T300A, I304M, I304R, 1304E, E306D, I309L, P310L, P310D, S311L, A314S, A314SHVRSV (“SHVRSV” disclosed as SEQ ID NO: 56), A314SHVRTV (“SHVRTV” disclosed as SEQ ID NO: 57), H315S, d315, d316, M316V, M316L, M316I, d317, R317H, R317N, R317Q, R317T, d318, A318Y, A318M, A318T, A318S, A318C, A318Q, A318R, A318H, A318V, A318W, A318I, A318L, d319, I319C, I319F, I319V, I319L, I319M, L320F, L320I, L320V, L320Y, L320T, L320M, L320W, S321C, A323G, A324S, A324M, L326V, L326I, L326C, G327S, A328H, A328V, A328R, A328S, A328RELE (“RELE” disclosed as SEQ ID NO: 58), N329E, d329, N329ELE, d330, V330L, V330T, V330I, d331, T331M, T331E, T331C, T331L, T331I, L334A, L334W, L334C, L334M, L334I, L335Q, L335R, T336K, V338M, T339S, N341H, N341T, K342L, K342F, E348D, E348H, E348Y, L351V, L351C, L351T, L351F, L351A, L351W, R352E, R352K, R352D, I353V, I353L, I353M, V354C, V354F, V354L, V354Y, V354A, V354K, V354S, V354I, Q355E, G356A, Y357W, L359M, T360S, T362C, T362S, S363A, S363T, S363G, P364C, P364S, V365L, V365I, V365M, V365C, A366S, A366V, A366T, T367A, T367C, T367S, T367H, L368H, F369H, F369Y, F369I, F369C, F369L, F369V, F369W, F369S, F369T, F369M, D370Y, D370R, D370T, D370S, D370H, D370G, D370N, D370Q, P371W, E372K, E372W, E372Q, E372H, E372S, E372R, E372N, D373S, D373H, D373P, D373Q, D373E, K374D, K374T, K374A, K374R, K374S, K374P, K374W, K374E, K374Q, K374H, E375P, R376T, R376K, R376E, R376Q, R376H, R376D, H377Y, H377R, H377N, I378S, 1378Y, 1378P, 1378V, 1378A, R379G, S380T, C381V, C381S, C381I, K383Q, K383H, K383T, K383Y, K383W, K383P, L384I, L384P, L384A, L384M, P386S, P386A, N387G, N387S, N387H, S388T, S388M, S388V, S388L, Q389R, Q389K, Q389E, V390C, V390L, V390A, R391K, N394D, V398D, V398N, A402V, A402L, A402I, D406L, D406V, P407D, L410N, L410Q, L410NTENAPR (“NTENAPR” disclosed as SEQ ID NO: 59), L410NTENAPD (“NTENAPD” disclosed as SEQ ID NO: 60), D411T, D411TENAPD (“TENAPD” disclosed as SEQ ID NO: 61), D411TQNAPR (“TQNAPR” disclosed as SEQ ID NO: 62), d411, D411TENAPR (“TENAPR” disclosed as SEQ ID NO: 63), D411TQNAPN (“TQNAPN” disclosed as SEQ ID NO: 64), d412, E412Q, d413, A413N, A413NNPN (“NNPN” disclosed as SEQ ID NO: 65), d414, I414A, I414S, I414N, d415, K415P, d416, Q416D, Q416R, Q416N, Q416E, T418P, L419G, P420E, L424M, L424V, W425V, W425L, I426M, I426L, Q430T, Q430N, Q430S, V431I, V431L, M432T, M432F, D433S, D433K, H436W, N442T, E443R, E443K, E443RASFA (“RASFA” disclosed as SEQ ID NO: 66), d444, d445, C445M, C445T, C445S, C445A, d446, d447, V447A, D455S, D455G, Y459G, Y459E, Y459D, R461D, R461G, H462R, H462G, F464L, F464Y, R465H, V469M, A470C, A470G, L471Y, L471K, L471M, L471W, L471I, L471R, V472I, V472F, V472C, V472Y, V472L, V472M, K474E, K474EDGH (“EDGH” disclosed as SEQ ID NO: 67), K474EDGY (“EDGY” disclosed as SEQ ID NO: 68), Q475D, d475, d476, d477, R477H, R477F, R477N, R477E, R477Y, Y478W, Y478I, Y478M, M479T, M479H, M479Y, M479F, M479W, I480V, I480L, V481C, V481I, V481T, T484L, E486D, K489I, S490R, S490C, S490V, Q491G, Q491R, Q491N, K493F, K493H, K493Y, K493L, Q494N, A496F, A496S, A496Y, M503L, M503V, M503I, G506R, G506RHPD (“RHPD” disclosed as SEQ ID NO: 69), d507, d508, A508P, d509, Q509D, T513A, T513V, T513C, I518V, Q519P, V521E, V521P, K523Q, K523A, K523R, K523M, K523Y, K523D, G524S, G524A, N525S, N525T, N525D, N525G, N525E, A527P, A527T, A527V, P528A, P528V, R529V, R529A, Y539L, Y539V, D540T, V542E, V542D, T546A, T546K, Q551K, Q551R, P553S, K554S, K554P, K554H, Y555F, Q557K, H559P, A560G, I562V, d562, V564F, I565V, I565F, I565FV, D566E, D566VE, A583Q, A583E or any combination thereof, in reference to SEQ ID NO: 1. It is understood that when multiple alterations without any delimitating characters (e.g., commas and/or spaces) are used in reference to an alteration at a specific position, the alteration is conjunctive and all identified alterations alter the corresponding specific position. For example, a G506RHPD (“RHPD” disclosed as SEQ ID NO: 69) means that R replaces the G at position 506 and HPD are inserted adjacent to the R. It is also understood that when a lower case “d” is used in reference to an alteration at a specific position, the alteration is a deletion at the specific position. For example the alteration d475 means that the residue at position 475 has been deleted.

In some embodiments, the recombinant nucleic acid encodes an engineered ACL having one or more amino acid alterations comprising: N7K, N7S, A8S, I10W, I10Y, I10F, N12E, N12P, N12D, V15I, D16E, Y19F, A52R, A52K, M54L, L64A, S66R, S67A, R73L, H74K, S82T, L87I, F90L, F90M, V94T, V100L, I111V, S115N, G117S, S119T, Q121D, V129I, T131G, A138D, A138S, K139E, A140K, A140E, A140D, V144T, K150L, K150I, A165S, L166F, M172L, M172A, M172D, R182Q, S191A, G199A, S209P, V212C, S217R, S221T, S221A, S223N, S223I, S223A, S223L, S223H, E224Q, N227E, N227T, N227A, A229I, A229L, A229M, S230Q, S230A, E231H, E231Q, E231P, S232Y, S232K, S232Q, S232N, L233W, L233Y, A234Y, A234F, A234H, T235S, Q238D, Q238H, Q238T, A240T, A240H, A240C, A240S, I243C, I243M, L249M, L249V, S253H, S253M, S253Q, K254M, L256I, L256V, G258T, G258H, G258A, G258L, G258M, N259A, A262K, A262C, A262N, Y268F, G271R, G271Q, I275E, V277L, V277M, Q281V, S288V, S288I, S288F, S288T, S288H, S288Y, T290V, T290A, I296L, I296M, V297I, T300A, I304E, E306D, M316L, A318S, A318R, A318H, A318W, A318Y, A318M, A318T, A318C, A318Q, A318V, A318I, A318L, L320M, L320F, L320T, L320Y, L320W, S321C, A323G, A328V, A328H, A328R, V330I, V330L, T331I, T336K, R352K, I353V, I353L, S363G, P364S, V365C, V365L, V365I, V365M, A366V, F369H, F369S, F369T, F369M, E372S, E372K, E372H, E372N, K374Q, K374R, K374T, K374P, K374H, K374S, K374A, K374D, K374E, R376T, I378A, I378P, I378S, I378V, S388T, V390C, V390A, N394D, V398N, D406L, P407D, Q416N, Q416E, M432F, E443R, E443K, C445A, D455S, D455G, H462R, V469M, A470G, L471R, L471M, L471W, I480V, V481I, V481C, T484L, E486D, S490C, Q491R, Q491G, K493L, K493H, K493Y, Q494N, A496Y, A508P, T513A, T513V, T513C, I518V, Q519P, V521E, V521P, K523R, K523Q, K523A, K523Y, K523M, K523D, N525D, N525S, N525T, N525G, N525E, T546K, K554H, K554S, K554P, Y555F, A560G, I565V, A583Q, A583E in reference to SEQ ID NO: 1.

In some embodiments, the recombinant nucleic acid encodes an engineered ACL having one or more amino acid alterations comprising: I10F, G199A, S223L, S223H, S223A, A229M, S230Q, S230A, E231P, S232N, L233W, L233Y, A234H, T235S, Q238T, I243C, I243M, L256I, L256V, G258L, G258M, V277M, S288Y, S288H, T300A, A318V, A318T, A318C, A318I, A318L, A318Q, A318M, A318Y, L320W, L320Y, S321C, A323G, A328R, V330L, P364S, V365I, V365M, F369M, E372N, K374E, I378V, H462R, V469M, I480V, V481C, T484L, T513C, T513A, T513V, K523D, N525G, N525E, N525T, K554S, K554P, A560G, or A583E, in reference to SEQ ID NO: 1.

In some embodiments, the recombinant nucleic acid encodes an engineered ACL having one or more amino acid alterations comprising: L233W, P364S, S232N, L320W, A318V, S321C, S223L, L320Y, T513C, Q238T, L233Y, K523D, A318T, S288Y, A318C, A234H, A323G, T513A, S288H, S223H, A318I, A318L, G258L, in reference to SEQ ID NO: 1.

In some embodiments, provided herein is a recombinant nucleic acid encoding an engineered ACL that is a variant of a reference polypeptide, wherein the reference polypeptide has an amino acid sequence of SEQ ID NO: 2, and the engineered ACL has one or more alterations at a position described in TABLE 9 relative to SEQ ID NO: 2. In some embodiments, the recombinant nucleic acid encodes an engineered ACL that includes one or more amino acid alterations at a residue corresponding to position 5, 6, 7, 8, 10, 12, 13, 14, 15, 16, 19, 20, 22, 25, 28, 37, 39, 40, 41, 43, 44, 45, 48, 50, 51, 53, 57, 58, 59, 61, 62, 63, 66, 67, 69, 71, 72, 74, 75, 76, 77, 78, 80, 81, 83, 84, 85, 86, 87, 88, 92, 93, 94, 95, 97, 98, 99, 100, 101, 103, 104, 106, 107, 108, 109, 110, 113, 114, 115, 116, 119, 120, 121, 122, 123, 124, 125, 126, 127, 128, 130, 131, 136, 137, 140, 141, 142, 145, 147, 148, 149, 150, 151, 157, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 216, 218, 219, 220, 221, 222, 224, 225, 226, 227, 228, 230, 231, 232, 233, 234, 235, 236, 237, 238, 239, 240, 241, 242, 243, 244, 245, 246, 247, 251, 252, 253, 254, 255, 256, 257, 259, 260, 262, 263, 264, 265, 266, 267, 268, 269, 270, 271, 272, 273, 274, 275, 277, 278, 279, 281, 282, 283, 284, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 341, 342, 343, 344, 345, 346, 347, 348, 349, 351, 352, 353, 354, 355, 357, 358, 359, 361, 362, 363, 365, 366, 367, 368, 369, 370, 372, 373, 375, 376, 377, 378, 379, 380, 381, 382, 383, 384, 385, 386, 387, 388, 389, 391, 392, 394, 395, 396, 398, 399, 400, 401, 402, 404, 405, 406, 407, 409, 411, 413, 414, 415, 416, 417, 418, 419, 420, 421, 425, 430, 432, 433, 434, 435, 441, 442, 443, 446, 447, 449, 450, 451, 452, 453, 455, 456, 458, 459, 460, 461, 462, 463, 464, 465, 466, 467, 468, 469, 470, 471, 473, 474, 476, 478, 479, 480, 481, 483, 485, 490, 492, 493, 496, 497, 498, 502, 504, 507, 508, 510, 511, 512, 513, 514, 515, 516, 518, 519, 521, 526, 527, 532, 535, 540, 542, 546, 552, 554, 558, 560, 561, 563, 564, 565, 566, 567, 568, 569, 570, 572, 573, 574, 575, 577, 578, 583, 584, 585, 587, 588, 591, 593, 595, 596, 598, 599, 601, more than one of the foregoing, or a combination thereof, in SEQ ID NO: 2. In some embodiments, the recombinant nucleic acid encodes an engineered ACL that includes one or more amino acid alterations at a residue corresponding to position 5, 6, 7, 8, 10, 12, 13, 14, 15, 16, 19, 20, 22, 25, 28, 37, 39, 40, 41, 43, 44, 45, 48, 50, 51, 53, 57, 58, 59, 61, 62, 63, 66, 67, 69, 71, 72, 74, 75, 76, 77, 78, 80, 81, 83, 84, 85, 86, 87, 88, 92, 93, 94, 95, 97, 98, 99, 100, 101, 103, 104, 106, 107, 108, 109, 110, 113, 114, 115, 116, 119, 120, 121, 122, 123, 124, 125, 126, 127, 128, 130, 131, 136, 137, 140, 141, 142, 145, 147, 148, 149, 150, 151, 157, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 216, 218, 219, 220, 221, 222, 224, 225, 226, 227, 228, 230, 231, 232, 233, 234, 235, 236, 237, 238, 239, 240, 241, 242, 243, 244, 245, 246, 247, 251, 252, 253, 254, 255, 256, 257, 259, 260, 262, 263, 264, 265, 266, 267, 268, 269, 270, 271, 272, 273, 274, 275, 277, 278, 279, 281, 282, 283, 284, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 341, 342, 343, 344, 345, 346, 347, 348, 349, 351, 352, 353, 354, 355, 357, 358, 359, 361, 362, 363, 365, 366, 367, 368, 369, 370, 372, 373, 375, 376, 377, 378, 379, 380, 381, 382, 383, 384, 385, 386, 387, 388, 389, 391, 392, 394, 395, 396, 398, 399, 400, 401, 402, 404, 405, 406, 407, 409, 411, 413, 414, 415, 416, 417, 418, 419, 420, 421, 425, 430, 432, 433, 434, 435, 441, 442, 443, 446, 447, 449, 450, 451, 452, 453, 455, 456, 458, 459, 460, 461, 462, 463, 464, 465, 466, 467, 468, 469, 470, 471, 473, 474, 476, 478, 479, 480, 481, 483, 485, 490, 492, 493, 496, 497, 498, 502, 504, 507, 508, 510, 511, 512, 513, 514, 515, 516, 518, 519, 521, 526, 527, 532, 535, 540, 542, 546, 552, 554, 558, 560, 561, 563, 564, 565, 566, 567, 568, 569, 570, 572, 573, 574, 575, 577, 578, 583, 584, 585, 587, 588, 591, 593, 595, 596, 598, 599, 601, more than one of the foregoing, or a combination thereof, in SEQ ID NO: 2. In some embodiments, the recombinant nucleic acid encodes an engineered ACL that includes one or more amino acid alterations at a residue corresponding to position 5, 6, 7, 8, 10, 12, 13, 14, 15, 16, 19, 20, 22, 25, 28, 37, 39, 40, 41, 43, 45, 50, 51, 53, 57, 58, 59, 61, 62, 63, 67, 69, 71, 72, 74, 75, 76, 77, 78, 80, 81, 83, 84, 85, 86, 87, 88, 92, 93, 94, 95, 97, 98, 99, 100, 101, 103, 104, 106, 108, 109, 110, 113, 116, 119, 120, 121, 123, 124, 125, 126, 127, 128, 130, 131, 136, 137, 140, 141, 142, 145, 147, 148, 149, 150, 151, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 218, 219, 220, 221, 222, 224, 226, 227, 228, 230, 232, 233, 234, 235, 236, 237, 238, 239, 240, 241, 242, 243, 244, 245, 246, 247, 252, 253, 254, 255, 256, 260, 262, 263, 264, 265, 266, 267, 268, 270, 271, 272, 273, 274, 275, 277, 278, 279, 281, 283, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 342, 343, 344, 345, 346, 347, 348, 349, 351, 352, 353, 354, 355, 357, 358, 359, 362, 363, 365, 366, 367, 368, 369, 370, 375, 376, 377, 378, 379, 380, 381, 382, 383, 384, 385, 386, 387, 388, 389, 391, 392, 394, 396, 398, 399, 400, 401, 404, 406, 407, 409, 411, 414, 416, 419, 420, 421, 425, 430, 432, 433, 434, 435, 441, 442, 443, 446, 447, 450, 451, 452, 453, 456, 460, 461, 462, 463, 465, 466, 467, 468, 469, 470, 471, 473, 476, 478, 479, 480, 481, 485, 490, 492, 498, 507, 508, 510, 511, 512, 514, 515, 516, 518, 519, 521, 526, 527, 532, 535, 540, 542, 546, 552, 554, 560, 561, 563, 564, 565, 566, 567, 568, 569, 570, 572, 573, 574, 575, 577, 578, 583, 584, 585, 587, 588, 591, 593, 595, 596, 598, 599, 601, more than one of the foregoing, or a combination thereof, in SEQ ID NO: 2. In some embodiments, the recombinant nucleic acid encodes an engineered ACL that includes one or more amino acid alterations at a residue corresponding to position 5, 6, 7, 8, 10, 12, 14, 15, 16, 19, 20, 22, 28, 39, 41, 43, 45, 50, 57, 58, 61, 62, 63, 67, 69, 71, 72, 76, 77, 78, 81, 84, 85, 86, 87, 92, 93, 94, 95, 98, 104, 108, 109, 113, 116, 119, 120, 123, 124, 125, 126, 127, 130, 131, 136, 140, 141, 142, 145, 147, 148, 149, 151, 159, 161, 163, 164, 167, 168, 169, 170, 173, 175, 176, 177, 181, 183, 185, 186, 187, 190, 191, 193, 194, 195, 201, 205, 209, 214, 218, 219, 220, 221, 226, 228, 230, 232, 234, 237, 238, 239, 241, 242, 243, 244, 246, 247, 253, 254, 260, 262, 263, 266, 267, 272, 273, 274, 278, 279, 281, 283, 285, 290, 291, 292, 295, 296, 298, 300, 302, 303, 304, 305, 307, 311, 313, 314, 315, 316, 318, 321, 322, 330, 333, 334, 336, 337, 338, 339, 340, 344, 345, 346, 347, 348, 349, 351, 352, 353, 354, 355, 357, 358, 359, 362, 363, 365, 367, 369, 375, 376, 379, 382, 383, 384, 386, 387, 388, 389, 399, 400, 401, 404, 407, 409, 416, 419, 420, 421, 430, 432, 433, 434, 441, 442, 446, 450, 452, 453, 456, 460, 461, 465, 466, 471, 473, 476, 479, 480, 481, 485, 490, 492, 507, 511, 514, 515, 516, 518, 519, 521, 526, 527, 532, 540, 542, 546, 554, 561, 564, 565, 566, 567, 568, 570, 572, 574, 575, 577, 584, 587, 595, 596, 599, more than one of the foregoing, or a combination thereof, in SEQ ID NO: 2. In some embodiments, the recombinant nucleic acid encodes an engineered ACL that includes one or more amino acid alterations at a residue corresponding to position 5, 6, 7, 8, 10, 12, 14, 15, 19, 20, 22, 41, 45, 50, 58, 62, 63, 67, 69, 72, 76, 77, 81, 86, 87, 92, 93, 94, 95, 98, 104, 108, 113, 116, 120, 123, 124, 125, 126, 127, 130, 136, 140, 141, 142, 145, 147, 148, 149, 151, 159, 161, 163, 164, 167, 168, 169, 175, 176, 177, 185, 186, 187, 190, 191, 193, 195, 201, 205, 209, 214, 219, 220, 221, 228, 230, 232, 234, 237, 238, 239, 241, 242, 243, 244, 246, 247, 253, 254, 260, 263, 266, 267, 272, 273, 274, 278, 279, 281, 283, 291, 292, 295, 298, 300, 302, 303, 304, 305, 307, 311, 313, 314, 315, 318, 321, 322, 330, 333, 334, 336, 337, 338, 340, 344, 348, 349, 351, 353, 354, 355, 357, 358, 359, 362, 363, 365, 367, 369, 376, 379, 382, 383, 386, 387, 388, 389, 399, 400, 401, 404, 407, 409, 420, 421, 430, 432, 433, 434, 442, 446, 450, 452, 453, 456, 460, 461, 465, 466, 473, 476, 479, 480, 481, 485, 492, 507, 514, 515, 516, 518, 519, 521, 526, 527, 532, 540, 542, 554, 564, 565, 567, 568, 570, 572, 575, 584, 587, 595, 596, more than one of the foregoing, or a combination thereof, in SEQ ID NO: 2.

In some embodiments, the recombinant nucleic acid encodes an engineered ACL having one or more alterations described in TABLE 9. Accordingly, in some embodiments, the recombinant nucleic acid encodes an engineered ACL having: R, E at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; K, D, P, N, R at a residue corresponding to position 10 in SEQ ID NO: 2; A, I, V, M at a residue corresponding to position 12 in SEQ ID NO: 2; C at a residue corresponding to position 13 in SEQ ID NO: 2; P, A, Q at a residue corresponding to position 14 in SEQ ID NO: 2; E, L, S, R at a residue corresponding to position 15 in SEQ ID NO: 2; C, L, I at a residue corresponding to position 16 in SEQ ID NO: 2; D, R at a residue corresponding to position 19 in SEQ ID NO: 2; H, K, R, D at a residue corresponding to position 20 in SEQ ID NO: 2; D, E at a residue corresponding to position 22 in SEQ ID NO: 2; N at a residue corresponding to position 25 in SEQ ID NO: 2; M at a residue corresponding to position 28 in SEQ ID NO: 2; C at a residue corresponding to position 37 in SEQ ID NO: 2; R at a residue corresponding to position 39 in SEQ ID NO: 2; T at a residue corresponding to position 40 in SEQ ID NO: 2; M, L, V, I at a residue corresponding to position 41 in SEQ ID NO: 2; S at a residue corresponding to position 43 in SEQ ID NO: 2; M at a residue corresponding to position 44 in SEQ ID NO: 2; I, V at a residue corresponding to position 45 in SEQ ID NO: 2; P at a residue corresponding to position 48 in SEQ ID NO: 2; I, F, R, V, L at a residue corresponding to position 50 in SEQ ID NO: 2; R at a residue corresponding to position 51 in SEQ ID NO: 2; L at a residue corresponding to position 53 in SEQ ID NO: 2; F at a residue corresponding to position 57 in SEQ ID NO: 2; V, L, M, F at a residue corresponding to position 58 in SEQ ID NO: 2; V at a residue corresponding to position 59 in SEQ ID NO: 2; P, E at a residue corresponding to position 61 in SEQ ID NO: 2; D at a residue corresponding to position 62 in SEQ ID NO: 2; D at a residue corresponding to position 63 in SEQ ID NO: 2; G at a residue corresponding to position 66 in SEQ ID NO: 2; K at a residue corresponding to position 67 in SEQ ID NO: 2; F, Y, L at a residue corresponding to position 69 in SEQ ID NO: 2; F, W at a residue corresponding to position 71 in SEQ ID NO: 2; A, R at a residue corresponding to position 72 in SEQ ID NO: 2; F at a residue corresponding to position 74 in SEQ ID NO: 2; D at a residue corresponding to position 75 in SEQ ID NO: 2; T, D, A, R at a residue corresponding to position 76 in SEQ ID NO: 2; R at a residue corresponding to position 77 in SEQ ID NO: 2; A at a residue corresponding to position 78 in SEQ ID NO: 2; K at a residue corresponding to position 80 in SEQ ID NO: 2; C at a residue corresponding to position 81 in SEQ ID NO: 2; S, H, Q, N at a residue corresponding to position 83 in SEQ ID NO: 2; Y, H at a residue corresponding to position 84 in SEQ ID NO: 2; F at a residue corresponding to position 85 in SEQ ID NO: 2; A, Q at a residue corresponding to position 86 in SEQ ID NO: 2; R at a residue corresponding to position 87 in SEQ ID NO: 2; L at a residue corresponding to position 88 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; A at a residue corresponding to position 97 in SEQ ID NO: 2; M at a residue corresponding to position 98 in SEQ ID NO: 2; L at a residue corresponding to position 99 in SEQ ID NO: 2; C at a residue corresponding to position 100 in SEQ ID NO: 2; P at a residue corresponding to position 101 in SEQ ID NO: 2; R at a residue corresponding to position 103 in SEQ ID NO: 2; L, W, A at a residue corresponding to position 104 in SEQ ID NO: 2; W, F at a residue corresponding to position 106 in SEQ ID NO: 2; V, P, I, L, W, F at a residue corresponding to position 107 in SEQ ID NO: 2; V at a residue corresponding to position 108 in SEQ ID NO: 2; V at a residue corresponding to position 109 in SEQ ID NO: 2; M, F, Y, A, D at a residue corresponding to position 110 in SEQ ID NO: 2; T, C, V at a residue corresponding to position 113 in SEQ ID NO: 2; M, W, L, G, A at a residue corresponding to position 114 in SEQ ID NO: 2; A at a residue corresponding to position 115 in SEQ ID NO: 2; I at a residue corresponding to position 116 in SEQ ID NO: 2; V, T, I at a residue corresponding to position 119 in SEQ ID NO: 2; M, F, V, A, L, W, Y at a residue corresponding to position 120 in SEQ ID NO: 2; T at a residue corresponding to position 121 in SEQ ID NO: 2; A at a residue corresponding to position 122 in SEQ ID NO: 2; M, V, I at a residue corresponding to position 123 in SEQ ID NO: 2; Y, S, N at a residue corresponding to position 124 in SEQ ID NO: 2; P, T, W at a residue corresponding to position 125 in SEQ ID NO: 2; M, N, R, A at a residue corresponding to position 126 in SEQ ID NO: 2; F, W, L at a residue corresponding to position 127 in SEQ ID NO: 2; R at a residue corresponding to position 128 in SEQ ID NO: 2; M at a residue corresponding to position 130 in SEQ ID NO: 2; E at a residue corresponding to position 131 in SEQ ID NO: 2; E, Q, K at a residue corresponding to position 136 in SEQ ID NO: 2; M at a residue corresponding to position 137 in SEQ ID NO: 2; M, C, I at a residue corresponding to position 140 in SEQ ID NO: 2; M at a residue corresponding to position 141 in SEQ ID NO: 2; N, Q at a residue corresponding to position 142 in SEQ ID NO: 2; G, R, D at a residue corresponding to position 145 in SEQ ID NO: 2; S at a residue corresponding to position 147 in SEQ ID NO: 2; L, V, A at a residue corresponding to position 148 in SEQ ID NO: 2; V, L at a residue corresponding to position 149 in SEQ ID NO: 2; V at a residue corresponding to position 150 in SEQ ID NO: 2; C at a residue corresponding to position 151 in SEQ ID NO: 2; L at a residue corresponding to position 157 in SEQ ID NO: 2; R, E at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 165 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, V, L at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 189 in SEQ ID NO: 2; V at a residue corresponding to position 190 in SEQ ID NO: 2; A, E at a residue corresponding to position 191 in SEQ ID NO: 2; T at a residue corresponding to position 192 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; P at a residue corresponding to position 196 in SEQ ID NO: 2; A, P, L at a residue corresponding to position 201 in SEQ ID NO: 2; T at a residue corresponding to position 205 in SEQ ID NO: 2; D at a residue corresponding to position 208 in SEQ ID NO: 2; I, T, P, V at a residue corresponding to position 209 in SEQ ID NO: 2; L, T, C, M, V, A at a residue corresponding to position 211 in SEQ ID NO: 2; L, M at a residue corresponding to position 212 in SEQ ID NO: 2; I, C, V, Q, M at a residue corresponding to position 213 in SEQ ID NO: 2; Y at a residue corresponding to position 214 in SEQ ID NO: 2; S at a residue corresponding to position 215 in SEQ ID NO: 2; G at a residue corresponding to position 216 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; T, M, L, R, K, H at a residue corresponding to position 221 in SEQ ID NO: 2; S at a residue corresponding to position 222 in SEQ ID NO: 2; A at a residue corresponding to position 224 in SEQ ID NO: 2; A at a residue corresponding to position 225 in SEQ ID NO: 2; M, I, V at a residue corresponding to position 226 in SEQ ID NO: 2; L, D, H, I at a residue corresponding to position 227 in SEQ ID NO: 2; S at a residue corresponding to position 228 in SEQ ID NO: 2; H, K at a residue corresponding to position 230 in SEQ ID NO: 2; N at a residue corresponding to position 231 in SEQ ID NO: 2; L, I, V at a residue corresponding to position 232 in SEQ ID NO: 2; L, V, M, W, A, I at a residue corresponding to position 233 in SEQ ID NO: 2; S, W at a residue corresponding to position 234 in SEQ ID NO: 2; Q, S at a residue corresponding to position 235 in SEQ ID NO: 2; A, C, I, M, V at a residue corresponding to position 236 in SEQ ID NO: 2; A, M, Q, R, E at a residue corresponding to position 237 in SEQ ID NO: 2; M, S, W, N, H at a residue corresponding to position 238 in SEQ ID NO: 2; F, M, V, C, L at a residue corresponding to position 239 in SEQ ID NO: 2; A, R at a residue corresponding to position 240 in SEQ ID NO: 2; A, H, E, Q at a residue corresponding to position 241 in SEQ ID NO: 2; V, C, T, W at a residue corresponding to position 242 in SEQ ID NO: 2; I, F, M, W, V at a residue corresponding to position 243 in SEQ ID NO: 2; E, P, Q, H, G at a residue corresponding to position 244 in SEQ ID NO: 2; M, L, H, W, Y at a residue corresponding to position 245 in SEQ ID NO: 2; I, T, M at a residue corresponding to position 246 in SEQ ID NO: 2; R, P, E at a residue corresponding to position 247 in SEQ ID NO: 2; E at a residue corresponding to position 251 in SEQ ID NO: 2; H, V at a residue corresponding to position 252 in SEQ ID NO: 2; Y, M, F, L, V at a residue corresponding to position 253 in SEQ ID NO: 2; I, F, V, M at a residue corresponding to position 254 in SEQ ID NO: 2; A, T, N, L, I, G, H, C, V, S at a residue corresponding to position 255 in SEQ ID NO: 2; F, V, I, C, Y at a residue corresponding to position 256 in SEQ ID NO: 2; M at a residue corresponding to position 257 in SEQ ID NO: 2; L, F at a residue corresponding to position 259 in SEQ ID NO: 2; H, S, Y, F at a residue corresponding to position 260 in SEQ ID NO: 2; C, T, A, I, M at a residue corresponding to position 262 in SEQ ID NO: 2; A, Y, H at a residue corresponding to position 263 in SEQ ID NO: 2; A at a residue corresponding to position 264 in SEQ ID NO: 2; M, L, W, F, C at a residue corresponding to position 265 in SEQ ID NO: 2; N, T, M, V at a residue corresponding to position 266 in SEQ ID NO: 2; I, G, M, W, F, L, V, C, A at a residue corresponding to position 267 in SEQ ID NO: 2; M, A, C, G, V at a residue corresponding to position 268 in SEQ ID NO: 2; L, M, I, V at a residue corresponding to position 269 in SEQ ID NO: 2; M, H, Y, T at a residue corresponding to position 270 in SEQ ID NO: 2; A, P, C, T, L at a residue corresponding to position 271 in SEQ ID NO: 2; C, V, P, A, T at a residue corresponding to position 272 in SEQ ID NO: 2; M, V, F, I at a residue corresponding to position 273 in SEQ ID NO: 2; Y, R, V, M, C, H, W at a residue corresponding to position 274 in SEQ ID NO: 2; G, C, S, H at a residue corresponding to position 275 in SEQ ID NO: 2; A, C at a residue corresponding to position 277 in SEQ ID NO: 2; C, T, A, H at a residue corresponding to position 278 in SEQ ID NO: 2; V, M, I at a residue corresponding to position 279 in SEQ ID NO: 2; F, L, V, M at a residue corresponding to position 281 in SEQ ID NO: 2; P, M, E, A, H at a residue corresponding to position 282 in SEQ ID NO: 2; E, D, P, R at a residue corresponding to position 283 in SEQ ID NO: 2; F, R, W at a residue corresponding to position 284 in SEQ ID NO: 2; R, F, W, D at a residue corresponding to position 285 in SEQ ID NO: 2; D, F at a residue corresponding to position 286 in SEQ ID NO: 2; P, A, V at a residue corresponding to position 287 in SEQ ID NO: 2; A at a residue corresponding to position 290 in SEQ ID NO: 2; M, V, L, P, I at a residue corresponding to position 291 in SEQ ID NO: 2; H at a residue corresponding to position 292 in SEQ ID NO: 2; I, L, A at a residue corresponding to position 294 in SEQ ID NO: 2; W, M, V, I at a residue corresponding to position 295 in SEQ ID NO: 2; D, Q at a residue corresponding to position 296 in SEQ ID NO: 2; M, V, L at a residue corresponding to position 298 in SEQ ID NO: 2; A at a residue corresponding to position 299 in SEQ ID NO: 2; Q at a residue corresponding to position 300 in SEQ ID NO: 2; Q at a residue corresponding to position 302 in SEQ ID NO: 2; V, I, A, P, C at a residue corresponding to position 303 in SEQ ID NO: 2; S at a residue corresponding to position 304 in SEQ ID NO: 2; W, Y, V, I, F, H, M at a residue corresponding to position 305 in SEQ ID NO: 2; V, Y, I, M, L, F at a residue corresponding to position 306 in SEQ ID NO: 2; P, W, Y, H, M at a residue corresponding to position 307 in SEQ ID NO: 2; T, A, S, L, G, V, M at a residue corresponding to position 308 in SEQ ID NO: 2; A, R, P, T at a residue corresponding to position 311 in SEQ ID NO: 2; I, M, V, F at a residue corresponding to position 312 in SEQ ID NO: 2; L, F, W at a residue corresponding to position 313 in SEQ ID NO: 2; Q, E, H at a residue corresponding to position 314 in SEQ ID NO: 2; R, A, K, L at a residue corresponding to position 315 in SEQ ID NO: 2; W, M, I, F at a residue corresponding to position 316 in SEQ ID NO: 2; H, D at a residue corresponding to position 318 in SEQ ID NO: 2; R, Q at a residue corresponding to position 321 in SEQ ID NO: 2; Q, K, A, H at a residue corresponding to position 322 in SEQ ID NO: 2; L, F at a residue corresponding to position 330 in SEQ ID NO: 2; G at a residue corresponding to position 331 in SEQ ID NO: 2; S at a residue corresponding to position 333 in SEQ ID NO: 2; I, M, V at a residue corresponding to position 334 in SEQ ID NO: 2; R, K at a residue corresponding to position 335 in SEQ ID NO: 2; T, I, M, Y, V, F, W at a residue corresponding to position 336 in SEQ ID NO: 2; L, M, F, A, V at a residue corresponding to position 337 in SEQ ID NO: 2; W, L, M, V at a residue corresponding to position 338 in SEQ ID NO: 2; F, T, S, C at a residue corresponding to position 339 in SEQ ID NO: 2; G at a residue corresponding to position 340 in SEQ ID NO: 2; A, S at a residue corresponding to position 341 in SEQ ID NO: 2; S at a residue corresponding to position 342 in SEQ ID NO: 2; P, A at a residue corresponding to position 343 in SEQ ID NO: 2; M, I, V, C at a residue corresponding to position 344 in SEQ ID NO: 2; S at a residue corresponding to position 345 in SEQ ID NO: 2; V, A, P, E at a residue corresponding to position 346 in SEQ ID NO: 2; E at a residue corresponding to position 347 in SEQ ID NO: 2; T, I, L at a residue corresponding to position 348 in SEQ ID NO: 2; M, F, L, A, I, Y at a residue corresponding to position 349 in SEQ ID NO: 2; F, R, Q, W at a residue corresponding to position 351 in SEQ ID NO: 2; I, M, Y at a residue corresponding to position 352 in SEQ ID NO: 2; R at a residue corresponding to position 353 in SEQ ID NO: 2; K at a residue corresponding to position 354 in SEQ ID NO: 2; A, R at a residue corresponding to position 355 in SEQ ID NO: 2; W, G, V, L, P, I, F, A at a residue corresponding to position 357 in SEQ ID NO: 2; Q at a residue corresponding to position 358 in SEQ ID NO: 2; H, Q at a residue corresponding to position 359 in SEQ ID NO: 2; F at a residue corresponding to position 361 in SEQ ID NO: 2; H, L, C at a residue corresponding to position 362 in SEQ ID NO: 2; D, V, I, G at a residue corresponding to position 363 in SEQ ID NO: 2; M, V, I at a residue corresponding to position 365 in SEQ ID NO: 2; I, V, L, F at a residue corresponding to position 366 in SEQ ID NO: 2; T, C, M, I, V, L at a residue corresponding to position 367 in SEQ ID NO: 2; N, Q, E, T at a residue corresponding to position 368 in SEQ ID NO: 2; G, A at a residue corresponding to position 369 in SEQ ID NO: 2; Y, F at a residue corresponding to position 370 in SEQ ID NO: 2; L, Q at a residue corresponding to position 372 in SEQ ID NO: 2; S at a residue corresponding to position 373 in SEQ ID NO: 2; A, C at a residue corresponding to position 375 in SEQ ID NO: 2; T, S, C, G at a residue corresponding to position 376 in SEQ ID NO: 2; G, M at a residue corresponding to position 377 in SEQ ID NO: 2; C, T, V at a residue corresponding to position 378 in SEQ ID NO: 2; C, V, I, L, A at a residue corresponding to position 379 in SEQ ID NO: 2; F, C, H, S, M, T, V at a residue corresponding to position 380 in SEQ ID NO: 2; N, F, L, C, V at a residue corresponding to position 381 in SEQ ID NO: 2; L, N, C, T, P at a residue corresponding to position 382 in SEQ ID NO: 2; D, N, R, P, Y at a residue corresponding to position 383 in SEQ ID NO: 2; P, N, D, W at a residue corresponding to position 384 in SEQ ID NO: 2; G, D at a residue corresponding to position 385 in SEQ ID NO: 2; D, V, L, T at a residue corresponding to position 386 in SEQ ID NO: 2; D, R, N, H, E at a residue corresponding to position 387 in SEQ ID NO: 2; K, H at a residue corresponding to position 388 in SEQ ID NO: 2; A, P, L at a residue corresponding to position 389 in SEQ ID NO: 2; S, T at a residue corresponding to position 391 in SEQ ID NO: 2; V, C at a residue corresponding to position 392 in SEQ ID NO: 2; R, K, I, Q, L at a residue corresponding to position 394 in SEQ ID NO: 2; A at a residue corresponding to position 395 in SEQ ID NO: 2; F, I, M, V at a residue corresponding to position 396 in SEQ ID NO: 2; D, H, N at a residue corresponding to position 398 in SEQ ID NO: 2; M, V, T, C, I at a residue corresponding to position 399 in SEQ ID NO: 2; D, Q at a residue corresponding to position 400 in SEQ ID NO: 2; M, I, C, L, A at a residue corresponding to position 401 in SEQ ID NO: 2; R at a residue corresponding to position 402 in SEQ ID NO: 2; V at a residue corresponding to position 404 in SEQ ID NO: 2; D at a residue corresponding to position 405 in SEQ ID NO: 2; E at a residue corresponding to position 406 in SEQ ID NO: 2; P at a residue corresponding to position 407 in SEQ ID NO: 2; Q, E, G at a residue corresponding to position 409 in SEQ ID NO: 2; D, S, V, G, N at a residue corresponding to position 411 in SEQ ID NO: 2; I, V at a residue corresponding to position 413 in SEQ ID NO: 2; W, L, V, C, Q at a residue corresponding to position 414 in SEQ ID NO: 2; I at a residue corresponding to position 415 in SEQ ID NO: 2; R at a residue corresponding to position 416 in SEQ ID NO: 2; S at a residue corresponding to position 417 in SEQ ID NO: 2; D at a residue corresponding to position 418 in SEQ ID NO: 2; H, S, Q, M at a residue corresponding to position 419 in SEQ ID NO: 2; V at a residue corresponding to position 420 in SEQ ID NO: 2; T, F at a residue corresponding to position 421 in SEQ ID NO: 2; H, L at a residue corresponding to position 425 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; T, V at a residue corresponding to position 434 in SEQ ID NO: 2; I, M, L at a residue corresponding to position 435 in SEQ ID NO: 2; F, M, Y at a residue corresponding to position 441 in SEQ ID NO: 2; K, H, Y, R, C at a residue corresponding to position 442 in SEQ ID NO: 2; S at a residue corresponding to position 443 in SEQ ID NO: 2; M, I, L, V at a residue corresponding to position 446 in SEQ ID NO: 2; A, G at a residue corresponding to position 447 in SEQ ID NO: 2; I at a residue corresponding to position 449 in SEQ ID NO: 2; D, M at a residue corresponding to position 450 in SEQ ID NO: 2; E at a residue corresponding to position 451 in SEQ ID NO: 2; P at a residue corresponding to position 452 in SEQ ID NO: 2; G at a residue corresponding to position 453 in SEQ ID NO: 2; E, G, R at a residue corresponding to position 455 in SEQ ID NO: 2; D, E, Q, N at a residue corresponding to position 456 in SEQ ID NO: 2; N, F, Y, W at a residue corresponding to position 458 in SEQ ID NO: 2; Y at a residue corresponding to position 459 in SEQ ID NO: 2; Y, F, I, R, W at a residue corresponding to position 460 in SEQ ID NO: 2; I, L, Y at a residue corresponding to position 461 in SEQ ID NO: 2; I, T, V at a residue corresponding to position 462 in SEQ ID NO: 2; D at a residue corresponding to position 463 in SEQ ID NO: 2; D at a residue corresponding to position 464 in SEQ ID NO: 2; L at a residue corresponding to position 465 in SEQ ID NO: 2; D, K, A at a residue corresponding to position 466 in SEQ ID NO: 2; K at a residue corresponding to position 467 in SEQ ID NO: 2; M at a residue corresponding to position 468 in SEQ ID NO: 2; L at a residue corresponding to position 469 in SEQ ID NO: 2; V at a residue corresponding to position 470 in SEQ ID NO: 2; T, V at a residue corresponding to position 471 in SEQ ID NO: 2; S, N at a residue corresponding to position 473 in SEQ ID NO: 2; E at a residue corresponding to position 474 in SEQ ID NO: 2; N, Y, L, F, V, I at a residue corresponding to position 476 in SEQ ID NO: 2; T, L at a residue corresponding to position 478 in SEQ ID NO: 2; P, V at a residue corresponding to position 479 in SEQ ID NO: 2; L, I, S, P, A, V at a residue corresponding to position 480 in SEQ ID NO: 2; P, E at a residue corresponding to position 481 in SEQ ID NO: 2; E at a residue corresponding to position 483 in SEQ ID NO: 2; A, L, E, R at a residue corresponding to position 485 in SEQ ID NO: 2; C at a residue corresponding to position 490 in SEQ ID NO: 2; P at a residue corresponding to position 492 in SEQ ID NO: 2; F at a residue corresponding to position 493 in SEQ ID NO: 2; H at a residue corresponding to position 496 in SEQ ID NO: 2; A at a residue corresponding to position 497 in SEQ ID NO: 2; C at a residue corresponding to position 498 in SEQ ID NO: 2; P at a residue corresponding to position 502 in SEQ ID NO: 2; H at a residue corresponding to position 504 in SEQ ID NO: 2; Y, V, W at a residue corresponding to position 507 in SEQ ID NO: 2; V at a residue corresponding to position 508 in SEQ ID NO: 2; A at a residue corresponding to position 510 in SEQ ID NO: 2; V, L at a residue corresponding to position 511 in SEQ ID NO: 2; I at a residue corresponding to position 512 in SEQ ID NO: 2; V at a residue corresponding to position 513 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; L, M at a residue corresponding to position 526 in SEQ ID NO: 2; L at a residue corresponding to position 527 in SEQ ID NO: 2; R, H at a residue corresponding to position 532 in SEQ ID NO: 2; L at a residue corresponding to position 535 in SEQ ID NO: 2; Q at a residue corresponding to position 540 in SEQ ID NO: 2; Y, W at a residue corresponding to position 542 in SEQ ID NO: 2; N at a residue corresponding to position 546 in SEQ ID NO: 2; Q at a residue corresponding to position 552 in SEQ ID NO: 2; E at a residue corresponding to position 554 in SEQ ID NO: 2; F at a residue corresponding to position 558 in SEQ ID NO: 2; D at a residue corresponding to position 560 in SEQ ID NO: 2; D at a residue corresponding to position 561 in SEQ ID NO: 2; F, L, W at a residue corresponding to position 563 in SEQ ID NO: 2; D, K, S, L, T, R at a residue corresponding to position 564 in SEQ ID NO: 2; M, I, V, P, T at a residue corresponding to position 565 in SEQ ID NO: 2; A, E, D, P at a residue corresponding to position 566 in SEQ ID NO: 2; K, Q, T, N, S, A at a residue corresponding to position 567 in SEQ ID NO: 2; G, N, D at a residue corresponding to position 568 in SEQ ID NO: 2; M, K at a residue corresponding to position 569 in SEQ ID NO: 2; V, L at a residue corresponding to position 570 in SEQ ID NO: 2; R, P at a residue corresponding to position 572 in SEQ ID NO: 2; T, S at a residue corresponding to position 573 in SEQ ID NO: 2; M, L at a residue corresponding to position 574 in SEQ ID NO: 2; A, Y, K, T at a residue corresponding to position 575 in SEQ ID NO: 2; Y, A, L at a residue corresponding to position 577 in SEQ ID NO: 2; Q, A at a residue corresponding to position 578 in SEQ ID NO: 2; D at a residue corresponding to position 583 in SEQ ID NO: 2; N at a residue corresponding to position 584 in SEQ ID NO: 2; H, L at a residue corresponding to position 585 in SEQ ID NO: 2; A at a residue corresponding to position 587 in SEQ ID NO: 2; Q at a residue corresponding to position 588 in SEQ ID NO: 2; Q, E at a residue corresponding to position 591 in SEQ ID NO: 2; A at a residue corresponding to position 593 in SEQ ID NO: 2; E at a residue corresponding to position 595 in SEQ ID NO: 2; K at a residue corresponding to position 596 in SEQ ID NO: 2; D at a residue corresponding to position 598 in SEQ ID NO: 2; S at a residue corresponding to position 599 in SEQ ID NO: 2; L at a residue corresponding to position 601 in SEQ ID NO: 2; more than one of the foregoing; or any combination thereof. It is understood that when alterations are listed herein for a certain position, the alteration can be in the alternative or in the conjunctive depending on the context. For example, the F, L, Y at a residue corresponding to position 69 in SEQ ID NO: 2, means that alterations at a residue corresponding to position 69 in SEQ ID NO: 2 can comprise F or L or Y.

In some embodiments, the recombinant nucleic acid encodes an engineered ACL having: E, R at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; D, N, R, P, K at a residue corresponding to position 10 in SEQ ID NO: 2; A, V, M, I at a residue corresponding to position 12 in SEQ ID NO: 2; C at a residue corresponding to position 13 in SEQ ID NO: 2; Q, P, A at a residue corresponding to position 14 in SEQ ID NO: 2; S, E at a residue corresponding to position 15 in SEQ ID NO: 2; I, C at a residue corresponding to position 16 in SEQ ID NO: 2; D, R at a residue corresponding to position 19 in SEQ ID NO: 2; H, K, R, D at a residue corresponding to position 20 in SEQ ID NO: 2; E, D at a residue corresponding to position 22 in SEQ ID NO: 2; N at a residue corresponding to position 25 in SEQ ID NO: 2; M at a residue corresponding to position 28 in SEQ ID NO: 2; C at a residue corresponding to position 37 in SEQ ID NO: 2; R at a residue corresponding to position 39 in SEQ ID NO: 2; T at a residue corresponding to position 40 in SEQ ID NO: 2; V, I, M at a residue corresponding to position 41 in SEQ ID NO: 2; S at a residue corresponding to position 43 in SEQ ID NO: 2; I, V at a residue corresponding to position 45 in SEQ ID NO: 2; R, L, I, V, F at a residue corresponding to position 50 in SEQ ID NO: 2; R at a residue corresponding to position 51 in SEQ ID NO: 2; L at a residue corresponding to position 53 in SEQ ID NO: 2; F at a residue corresponding to position 57 in SEQ ID NO: 2; F, M, L, V at a residue corresponding to position 58 in SEQ ID NO: 2; V at a residue corresponding to position 59 in SEQ ID NO: 2; P at a residue corresponding to position 61 in SEQ ID NO: 2; D at a residue corresponding to position 62 in SEQ ID NO: 2; D at a residue corresponding to position 63 in SEQ ID NO: 2; K at a residue corresponding to position 67 in SEQ ID NO: 2; F, L, Y at a residue corresponding to position 69 in SEQ ID NO: 2; W, F at a residue corresponding to position 71 in SEQ ID NO: 2; A, R at a residue corresponding to position 72 in SEQ ID NO: 2; F at a residue corresponding to position 74 in SEQ ID NO: 2; D at a residue corresponding to position 75 in SEQ ID NO: 2; D, T, A, R at a residue corresponding to position 76 in SEQ ID NO: 2; R at a residue corresponding to position 77 in SEQ ID NO: 2; A at a residue corresponding to position 78 in SEQ ID NO: 2; K at a residue corresponding to position 80 in SEQ ID NO: 2; C at a residue corresponding to position 81 in SEQ ID NO: 2; S, N at a residue corresponding to position 83 in SEQ ID NO: 2; Y, H at a residue corresponding to position 84 in SEQ ID NO: 2; F at a residue corresponding to position 85 in SEQ ID NO: 2; Q, A at a residue corresponding to position 86 in SEQ ID NO: 2; R at a residue corresponding to position 87 in SEQ ID NO: 2; L at a residue corresponding to position 88 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; A at a residue corresponding to position 97 in SEQ ID NO: 2; M at a residue corresponding to position 98 in SEQ ID NO: 2; L at a residue corresponding to position 99 in SEQ ID NO: 2; C at a residue corresponding to position 100 in SEQ ID NO: 2; P at a residue corresponding to position 101 in SEQ ID NO: 2; R at a residue corresponding to position 103 in SEQ ID NO: 2; A, L, W at a residue corresponding to position 104 in SEQ ID NO: 2; F at a residue corresponding to position 106 in SEQ ID NO: 2; V at a residue corresponding to position 108 in SEQ ID NO: 2; V at a residue corresponding to position 109 in SEQ ID NO: 2; A, M at a residue corresponding to position 110 in SEQ ID NO: 2; C at a residue corresponding to position 113 in SEQ ID NO: 2; I at a residue corresponding to position 116 in SEQ ID NO: 2; T, V, I at a residue corresponding to position 119 in SEQ ID NO: 2; Y, V, F, L, M, A at a residue corresponding to position 120 in SEQ ID NO: 2; T at a residue corresponding to position 121 in SEQ ID NO: 2; I, M, V at a residue corresponding to position 123 in SEQ ID NO: 2; S, N, Y at a residue corresponding to position 124 in SEQ ID NO: 2; P, T, W at a residue corresponding to position 125 in SEQ ID NO: 2; R, A, N, M at a residue corresponding to position 126 in SEQ ID NO: 2; L, W, F at a residue corresponding to position 127 in SEQ ID NO: 2; R at a residue corresponding to position 128 in SEQ ID NO: 2; M at a residue corresponding to position 130 in SEQ ID NO: 2; E at a residue corresponding to position 131 in SEQ ID NO: 2; E, K, Q at a residue corresponding to position 136 in SEQ ID NO: 2; M at a residue corresponding to position 137 in SEQ ID NO: 2; M, I, C at a residue corresponding to position 140 in SEQ ID NO: 2; M at a residue corresponding to position 141 in SEQ ID NO: 2; Q, N at a residue corresponding to position 142 in SEQ ID NO: 2; R, D, G at a residue corresponding to position 145 in SEQ ID NO: 2; S at a residue corresponding to position 147 in SEQ ID NO: 2; V, L, A at a residue corresponding to position 148 in SEQ ID NO: 2; V at a residue corresponding to position 149 in SEQ ID NO: 2; V at a residue corresponding to position 150 in SEQ ID NO: 2; C at a residue corresponding to position 151 in SEQ ID NO: 2; E, R at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 165 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, L, V at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 189 in SEQ ID NO: 2; V at a residue corresponding to position 190 in SEQ ID NO: 2; E, A at a residue corresponding to position 191 in SEQ ID NO: 2; T at a residue corresponding to position 192 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; P at a residue corresponding to position 196 in SEQ ID NO: 2; L, A at a residue corresponding to position 201 in SEQ ID NO: 2; T at a residue corresponding to position 205 in SEQ ID NO: 2; D at a residue corresponding to position 208 in SEQ ID NO: 2; I, T, V at a residue corresponding to position 209 in SEQ ID NO: 2; L at a residue corresponding to position 211 in SEQ ID NO: 2; L, M at a residue corresponding to position 212 in SEQ ID NO: 2; I, M, V at a residue corresponding to position 213 in SEQ ID NO: 2; Y at a residue corresponding to position 214 in SEQ ID NO: 2; S at a residue corresponding to position 215 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; K, L, T, H, R, M at a residue corresponding to position 221 in SEQ ID NO: 2; S at a residue corresponding to position 222 in SEQ ID NO: 2; A at a residue corresponding to position 224 in SEQ ID NO: 2; M, V, I at a residue corresponding to position 226 in SEQ ID NO: 2; I at a residue corresponding to position 227 in SEQ ID NO: 2; S at a residue corresponding to position 228 in SEQ ID NO: 2; H, K at a residue corresponding to position 230 in SEQ ID NO: 2; L at a residue corresponding to position 232 in SEQ ID NO: 2; A at a residue corresponding to position 233 in SEQ ID NO: 2; S at a residue corresponding to position 234 in SEQ ID NO: 2; Q at a residue corresponding to position 235 in SEQ ID NO: 2; M at a residue corresponding to position 236 in SEQ ID NO: 2; A, M, Q at a residue corresponding to position 237 in SEQ ID NO: 2; M, H at a residue corresponding to position 238 in SEQ ID NO: 2; V, L, M at a residue corresponding to position 239 in SEQ ID NO: 2; A at a residue corresponding to position 240 in SEQ ID NO: 2; Q, E, H, A at a residue corresponding to position 241 in SEQ ID NO: 2; C, V, T at a residue corresponding to position 242 in SEQ ID NO: 2; W, F, M, I, V at a residue corresponding to position 243 in SEQ ID NO: 2; P, Q, G, H, E at a residue corresponding to position 244 in SEQ ID NO: 2; L, M, W at a residue corresponding to position 245 in SEQ ID NO: 2; I at a residue corresponding to position 246 in SEQ ID NO: 2; R, P at a residue corresponding to position 247 in SEQ ID NO: 2; V, H at a residue corresponding to position 252 in SEQ ID NO: 2; L, V, M at a residue corresponding to position 253 in SEQ ID NO: 2; M, I, V at a residue corresponding to position 254 in SEQ ID NO: 2; C, V, T, N at a residue corresponding to position 255 in SEQ ID NO: 2; Y, V, F at a residue corresponding to position 256 in SEQ ID NO: 2; S, H, F, Y at a residue corresponding to position 260 in SEQ ID NO: 2; T, C, A at a residue corresponding to position 262 in SEQ ID NO: 2; Y at a residue corresponding to position 263 in SEQ ID NO: 2; A at a residue corresponding to position 264 in SEQ ID NO: 2; M at a residue corresponding to position 265 in SEQ ID NO: 2; N, M at a residue corresponding to position 266 in SEQ ID NO: 2; M, F, L, W, G at a residue corresponding to position 267 in SEQ ID NO: 2; M, A at a residue corresponding to position 268 in SEQ ID NO: 2; M at a residue corresponding to position 270 in SEQ ID NO: 2; L, C, A at a residue corresponding to position 271 in SEQ ID NO: 2; A, V, T, P, C at a residue corresponding to position 272 in SEQ ID NO: 2; I, V, M, F at a residue corresponding to position 273 in SEQ ID NO: 2; C, M, H, R, Y at a residue corresponding to position 274 in SEQ ID NO: 2; H, C at a residue corresponding to position 275 in SEQ ID NO: 2; A at a residue corresponding to position 277 in SEQ ID NO: 2; T, A, C at a residue corresponding to position 278 in SEQ ID NO: 2; M, I, V at a residue corresponding to position 279 in SEQ ID NO: 2; V, L at a residue corresponding to position 281 in SEQ ID NO: 2; E, D, P, R at a residue corresponding to position 283 in SEQ ID NO: 2; R, F, D at a residue corresponding to position 285 in SEQ ID NO: 2; D, F at a residue corresponding to position 286 in SEQ ID NO: 2; A, P, V at a residue corresponding to position 287 in SEQ ID NO: 2; A at a residue corresponding to position 290 in SEQ ID NO: 2; M, I, L, V at a residue corresponding to position 291 in SEQ ID NO: 2; H at a residue corresponding to position 292 in SEQ ID NO: 2; A at a residue corresponding to position 294 in SEQ ID NO: 2; V, I, M at a residue corresponding to position 295 in SEQ ID NO: 2; Q, D at a residue corresponding to position 296 in SEQ ID NO: 2; M, V, L at a residue corresponding to position 298 in SEQ ID NO: 2; A at a residue corresponding to position 299 in SEQ ID NO: 2; Q at a residue corresponding to position 300 in SEQ ID NO: 2; Q at a residue corresponding to position 302 in SEQ ID NO: 2; A, P, C, V at a residue corresponding to position 303 in SEQ ID NO: 2; S at a residue corresponding to position 304 in SEQ ID NO: 2; V, M, I at a residue corresponding to position 305 in SEQ ID NO: 2; I, V, Y at a residue corresponding to position 306 in SEQ ID NO: 2; M, W, P at a residue corresponding to position 307 in SEQ ID NO: 2; G at a residue corresponding to position 308 in SEQ ID NO: 2; T, A, R at a residue corresponding to position 311 in SEQ ID NO: 2; F, V, M at a residue corresponding to position 312 in SEQ ID NO: 2; F, W at a residue corresponding to position 313 in SEQ ID NO: 2; Q, H, E at a residue corresponding to position 314 in SEQ ID NO: 2; A, K, L, R at a residue corresponding to position 315 in SEQ ID NO: 2; I, M at a residue corresponding to position 316 in SEQ ID NO: 2; D, H at a residue corresponding to position 318 in SEQ ID NO: 2; R, Q at a residue corresponding to position 321 in SEQ ID NO: 2; A, K, H, Q at a residue corresponding to position 322 in SEQ ID NO: 2; F at a residue corresponding to position 330 in SEQ ID NO: 2; G at a residue corresponding to position 331 in SEQ ID NO: 2; S at a residue corresponding to position 333 in SEQ ID NO: 2; V, I, M at a residue corresponding to position 334 in SEQ ID NO: 2; K at a residue corresponding to position 335 in SEQ ID NO: 2; V, T, M, Y, W, I, F at a residue corresponding to position 336 in SEQ ID NO: 2; L, V, M at a residue corresponding to position 337 in SEQ ID NO: 2; L, M, V, W at a residue corresponding to position 338 in SEQ ID NO: 2; T, S, F at a residue corresponding to position 339 in SEQ ID NO: 2; G at a residue corresponding to position 340 in SEQ ID NO: 2; S at a residue corresponding to position 342 in SEQ ID NO: 2; P, A at a residue corresponding to position 343 in SEQ ID NO: 2; I, C, M, V at a residue corresponding to position 344 in SEQ ID NO: 2; S at a residue corresponding to position 345 in SEQ ID NO: 2; E, V, A, P at a residue corresponding to position 346 in SEQ ID NO: 2; E at a residue corresponding to position 347 in SEQ ID NO: 2; I, L, T at a residue corresponding to position 348 in SEQ ID NO: 2; Y, F, L at a residue corresponding to position 349 in SEQ ID NO: 2; R, Q, W at a residue corresponding to position 351 in SEQ ID NO: 2; I, M at a residue corresponding to position 352 in SEQ ID NO: 2; R at a residue corresponding to position 353 in SEQ ID NO: 2; K at a residue corresponding to position 354 in SEQ ID NO: 2; A at a residue corresponding to position 355 in SEQ ID NO: 2; F, I, V, A, G, L, W at a residue corresponding to position 357 in SEQ ID NO: 2; Q at a residue corresponding to position 358 in SEQ ID NO: 2; Q at a residue corresponding to position 359 in SEQ ID NO: 2; C, H, L at a residue corresponding to position 362 in SEQ ID NO: 2; V, I, G at a residue corresponding to position 363 in SEQ ID NO: 2; M, I, V at a residue corresponding to position 365 in SEQ ID NO: 2; I, L, F, V at a residue corresponding to position 366 in SEQ ID NO: 2; V, I, L, M, T, C at a residue corresponding to position 367 in SEQ ID NO: 2; N at a residue corresponding to position 368 in SEQ ID NO: 2; A, G at a residue corresponding to position 369 in SEQ ID NO: 2; Y at a residue corresponding to position 370 in SEQ ID NO: 2; C, A at a residue corresponding to position 375 in SEQ ID NO: 2; G, S at a residue corresponding to position 376 in SEQ ID NO: 2; G at a residue corresponding to position 377 in SEQ ID NO: 2; T at a residue corresponding to position 378 in SEQ ID NO: 2; V, C, L, A, I at a residue corresponding to position 379 in SEQ ID NO: 2; S, V, H, T at a residue corresponding to position 380 in SEQ ID NO: 2; L, C at a residue corresponding to position 381 in SEQ ID NO: 2; T, C, N, L, P at a residue corresponding to position 382 in SEQ ID NO: 2; Y, R at a residue corresponding to position 383 in SEQ ID NO: 2; N, W, D at a residue corresponding to position 384 in SEQ ID NO: 2; G at a residue corresponding to position 385 in SEQ ID NO: 2; V, T, L at a residue corresponding to position 386 in SEQ ID NO: 2; R, H, N, D, E at a residue corresponding to position 387 in SEQ ID NO: 2; K, H at a residue corresponding to position 388 in SEQ ID NO: 2; A, P, L at a residue corresponding to position 389 in SEQ ID NO: 2; S, T at a residue corresponding to position 391 in SEQ ID NO: 2; V at a residue corresponding to position 392 in SEQ ID NO: 2; I, K at a residue corresponding to position 394 in SEQ ID NO: 2; V at a residue corresponding to position 396 in SEQ ID NO: 2; H at a residue corresponding to position 398 in SEQ ID NO: 2; C, I, T, V at a residue corresponding to position 399 in SEQ ID NO: 2; Q, D at a residue corresponding to position 400 in SEQ ID NO: 2; L, I, C, A, M at a residue corresponding to position 401 in SEQ ID NO: 2; V at a residue corresponding to position 404 in SEQ ID NO: 2; E at a residue corresponding to position 406 in SEQ ID NO: 2; P at a residue corresponding to position 407 in SEQ ID NO: 2; G, Q, E at a residue corresponding to position 409 in SEQ ID NO: 2; S at a residue corresponding to position 411 in SEQ ID NO: 2; L, C at a residue corresponding to position 414 in SEQ ID NO: 2; R at a residue corresponding to position 416 in SEQ ID NO: 2; Q, H, M, S at a residue corresponding to position 419 in SEQ ID NO: 2; V at a residue corresponding to position 420 in SEQ ID NO: 2; F, T at a residue corresponding to position 421 in SEQ ID NO: 2; L, H at a residue corresponding to position 425 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; V, T at a residue corresponding to position 434 in SEQ ID NO: 2; M at a residue corresponding to position 435 in SEQ ID NO: 2; F, M at a residue corresponding to position 441 in SEQ ID NO: 2; Y, R, K, C, H at a residue corresponding to position 442 in SEQ ID NO: 2; S at a residue corresponding to position 443 in SEQ ID NO: 2; L, V, M, I at a residue corresponding to position 446 in SEQ ID NO: 2; A at a residue corresponding to position 447 in SEQ ID NO: 2; M, D at a residue corresponding to position 450 in SEQ ID NO: 2; E at a residue corresponding to position 451 in SEQ ID NO: 2; P at a residue corresponding to position 452 in SEQ ID NO: 2; G at a residue corresponding to position 453 in SEQ ID NO: 2; N, D at a residue corresponding to position 456 in SEQ ID NO: 2; F, I, R, W, Y at a residue corresponding to position 460 in SEQ ID NO: 2; Y, L at a residue corresponding to position 461 in SEQ ID NO: 2; V, I at a residue corresponding to position 462 in SEQ ID NO: 2; D at a residue corresponding to position 463 in SEQ ID NO: 2; L at a residue corresponding to position 465 in SEQ ID NO: 2; A, D, K at a residue corresponding to position 466 in SEQ ID NO: 2; K at a residue corresponding to position 467 in SEQ ID NO: 2; M at a residue corresponding to position 468 in SEQ ID NO: 2; L at a residue corresponding to position 469 in SEQ ID NO: 2; V at a residue corresponding to position 470 in SEQ ID NO: 2; T, V at a residue corresponding to position 471 in SEQ ID NO: 2; N, S at a residue corresponding to position 473 in SEQ ID NO: 2; Y, F, V, L, I, N at a residue corresponding to position 476 in SEQ ID NO: 2; T at a residue corresponding to position 478 in SEQ ID NO: 2; V, P at a residue corresponding to position 479 in SEQ ID NO: 2; S, A, P, L at a residue corresponding to position 480 in SEQ ID NO: 2; P, E at a residue corresponding to position 481 in SEQ ID NO: 2; L, E, R, A at a residue corresponding to position 485 in SEQ ID NO: 2; C at a residue corresponding to position 490 in SEQ ID NO: 2; P at a residue corresponding to position 492 in SEQ ID NO: 2; C at a residue corresponding to position 498 in SEQ ID NO: 2; V at a residue corresponding to position 507 in SEQ ID NO: 2; V at a residue corresponding to position 508 in SEQ ID NO: 2; A at a residue corresponding to position 510 in SEQ ID NO: 2; V, L at a residue corresponding to position 511 in SEQ ID NO: 2; I at a residue corresponding to position 512 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; M, L at a residue corresponding to position 526 in SEQ ID NO: 2; L at a residue corresponding to position 527 in SEQ ID NO: 2; R, H at a residue corresponding to position 532 in SEQ ID NO: 2; L at a residue corresponding to position 535 in SEQ ID NO: 2; Q at a residue corresponding to position 540 in SEQ ID NO: 2; Y, W at a residue corresponding to position 542 in SEQ ID NO: 2; N at a residue corresponding to position 546 in SEQ ID NO: 2; Q at a residue corresponding to position 552 in SEQ ID NO: 2; E at a residue corresponding to position 554 in SEQ ID NO: 2; D at a residue corresponding to position 560 in SEQ ID NO: 2; D at a residue corresponding to position 561 in SEQ ID NO: 2; L, F at a residue corresponding to position 563 in SEQ ID NO: 2; S, T, L, D, R, K at a residue corresponding to position 564 in SEQ ID NO: 2; T, M, P, V, I at a residue corresponding to position 565 in SEQ ID NO: 2; D, A, E, P at a residue corresponding to position 566 in SEQ ID NO: 2; A, S, Q, N, T, K at a residue corresponding to position 567 in SEQ ID NO: 2; G, D, N at a residue corresponding to position 568 in SEQ ID NO: 2; M, K at a residue corresponding to position 569 in SEQ ID NO: 2; V, L at a residue corresponding to position 570 in SEQ ID NO: 2; P at a residue corresponding to position 572 in SEQ ID NO: 2; S, T at a residue corresponding to position 573 in SEQ ID NO: 2; M, L at a residue corresponding to position 574 in SEQ ID NO: 2; T, Y at a residue corresponding to position 575 in SEQ ID NO: 2; A, L at a residue corresponding to position 577 in SEQ ID NO: 2; A, Q at a residue corresponding to position 578 in SEQ ID NO: 2; D at a residue corresponding to position 583 in SEQ ID NO: 2; N at a residue corresponding to position 584 in SEQ ID NO: 2; H at a residue corresponding to position 585 in SEQ ID NO: 2; A at a residue corresponding to position 587 in SEQ ID NO: 2; Q at a residue corresponding to position 588 in SEQ ID NO: 2; Q, E at a residue corresponding to position 591 in SEQ ID NO: 2; A at a residue corresponding to position 593 in SEQ ID NO: 2; E at a residue corresponding to position 595 in SEQ ID NO: 2; K at a residue corresponding to position 596 in SEQ ID NO: 2; D at a residue corresponding to position 598 in SEQ ID NO: 2; S at a residue corresponding to position 599 in SEQ ID NO: 2; L at a residue corresponding to position 601 in SEQ ID NO: 2; or any combination thereof. It is understood that when alterations are listed herein for a certain position, the alteration can be in the alternative or in the conjunctive depending on the context. For example, the F, L, Y at a residue corresponding to position 69 in SEQ ID NO: 2, means that alterations at a residue corresponding to position 69 in SEQ ID NO: 2 can comprise F or L or Y.

In some embodiments, the recombinant nucleic acid encodes an engineered ACL having: E, R at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; D, N, R, P at a residue corresponding to position 10 in SEQ ID NO: 2; A, V, M at a residue corresponding to position 12 in SEQ ID NO: 2; Q, P at a residue corresponding to position 14 in SEQ ID NO: 2; S, E at a residue corresponding to position 15 in SEQ ID NO: 2; I at a residue corresponding to position 16 in SEQ ID NO: 2; D, R at a residue corresponding to position 19 in SEQ ID NO: 2; H, K, R, D at a residue corresponding to position 20 in SEQ ID NO: 2; E, D at a residue corresponding to position 22 in SEQ ID NO: 2; M at a residue corresponding to position 28 in SEQ ID NO: 2; R at a residue corresponding to position 39 in SEQ ID NO: 2; V at a residue corresponding to position 41 in SEQ ID NO: 2; S at a residue corresponding to position 43 in SEQ ID NO: 2; I at a residue corresponding to position 45 in SEQ ID NO: 2; R, L, I, V, F at a residue corresponding to position 50 in SEQ ID NO: 2; F at a residue corresponding to position 57 in SEQ ID NO: 2; F, M, L, V at a residue corresponding to position 58 in SEQ ID NO: 2; P at a residue corresponding to position 61 in SEQ ID NO: 2; D at a residue corresponding to position 62 in SEQ ID NO: 2; D at a residue corresponding to position 63 in SEQ ID NO: 2; K at a residue corresponding to position 67 in SEQ ID NO: 2; F, L, Y at a residue corresponding to position 69 in SEQ ID NO: 2; W, F at a residue corresponding to position 71 in SEQ ID NO: 2; A, R at a residue corresponding to position 72 in SEQ ID NO: 2; D, T, A, R at a residue corresponding to position 76 in SEQ ID NO: 2; R at a residue corresponding to position 77 in SEQ ID NO: 2; A at a residue corresponding to position 78 in SEQ ID NO: 2; C at a residue corresponding to position 81 in SEQ ID NO: 2; Y at a residue corresponding to position 84 in SEQ ID NO: 2; F at a residue corresponding to position 85 in SEQ ID NO: 2; Q at a residue corresponding to position 86 in SEQ ID NO: 2; R at a residue corresponding to position 87 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; M at a residue corresponding to position 98 in SEQ ID NO: 2; A at a residue corresponding to position 104 in SEQ ID NO: 2; V at a residue corresponding to position 108 in SEQ ID NO: 2; V at a residue corresponding to position 109 in SEQ ID NO: 2; C at a residue corresponding to position 113 in SEQ ID NO: 2; I at a residue corresponding to position 116 in SEQ ID NO: 2; T at a residue corresponding to position 119 in SEQ ID NO: 2; Y, V, F, L, M, A at a residue corresponding to position 120 in SEQ ID NO: 2; I at a residue corresponding to position 123 in SEQ ID NO: 2; S, N at a residue corresponding to position 124 in SEQ ID NO: 2; P, T, W at a residue corresponding to position 125 in SEQ ID NO: 2; R, A, N at a residue corresponding to position 126 in SEQ ID NO: 2; L, W at a residue corresponding to position 127 in SEQ ID NO: 2; M at a residue corresponding to position 130 in SEQ ID NO: 2; E at a residue corresponding to position 131 in SEQ ID NO: 2; E, K at a residue corresponding to position 136 in SEQ ID NO: 2; M, I, C at a residue corresponding to position 140 in SEQ ID NO: 2; M at a residue corresponding to position 141 in SEQ ID NO: 2; Q, N at a residue corresponding to position 142 in SEQ ID NO: 2; R at a residue corresponding to position 145 in SEQ ID NO: 2; S at a residue corresponding to position 147 in SEQ ID NO: 2; V at a residue corresponding to position 148 in SEQ ID NO: 2; V at a residue corresponding to position 149 in SEQ ID NO: 2; C at a residue corresponding to position 151 in SEQ ID NO: 2; E, R at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, L, V at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 190 in SEQ ID NO: 2; E, A at a residue corresponding to position 191 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; L, A at a residue corresponding to position 201 in SEQ ID NO: 2; T at a residue corresponding to position 205 in SEQ ID NO: 2; I at a residue corresponding to position 209 in SEQ ID NO: 2; Y at a residue corresponding to position 214 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; K, L, T, H at a residue corresponding to position 221 in SEQ ID NO: 2; M, V at a residue corresponding to position 226 in SEQ ID NO: 2; S at a residue corresponding to position 228 in SEQ ID NO: 2; H, K at a residue corresponding to position 230 in SEQ ID NO: 2; L at a residue corresponding to position 232 in SEQ ID NO: 2; S at a residue corresponding to position 234 in SEQ ID NO: 2; A at a residue corresponding to position 237 in SEQ ID NO: 2; M at a residue corresponding to position 238 in SEQ ID NO: 2; V at a residue corresponding to position 239 in SEQ ID NO: 2; Q, E at a residue corresponding to position 241 in SEQ ID NO: 2; C, V, T at a residue corresponding to position 242 in SEQ ID NO: 2; W, F, M at a residue corresponding to position 243 in SEQ ID NO: 2; P, Q at a residue corresponding to position 244 in SEQ ID NO: 2; I at a residue corresponding to position 246 in SEQ ID NO: 2; R at a residue corresponding to position 247 in SEQ ID NO: 2; L, V at a residue corresponding to position 253 in SEQ ID NO: 2; M, I, V at a residue corresponding to position 254 in SEQ ID NO: 2; S, H at a residue corresponding to position 260 in SEQ ID NO: 2; T at a residue corresponding to position 262 in SEQ ID NO: 2; Y at a residue corresponding to position 263 in SEQ ID NO: 2; N at a residue corresponding to position 266 in SEQ ID NO: 2; M, F at a residue corresponding to position 267 in SEQ ID NO: 2; A at a residue corresponding to position 272 in SEQ ID NO: 2; I, V, M, F at a residue corresponding to position 273 in SEQ ID NO: 2; C at a residue corresponding to position 274 in SEQ ID NO: 2; T, A at a residue corresponding to position 278 in SEQ ID NO: 2; M at a residue corresponding to position 279 in SEQ ID NO: 2; V at a residue corresponding to position 281 in SEQ ID NO: 2; E at a residue corresponding to position 283 in SEQ ID NO: 2; R at a residue corresponding to position 285 in SEQ ID NO: 2; A at a residue corresponding to position 290 in SEQ ID NO: 2; M, I, L, V at a residue corresponding to position 291 in SEQ ID NO: 2; H at a residue corresponding to position 292 in SEQ ID NO: 2; V, I, M at a residue corresponding to position 295 in SEQ ID NO: 2; Q at a residue corresponding to position 296 in SEQ ID NO: 2; M, V at a residue corresponding to position 298 in SEQ ID NO: 2; Q at a residue corresponding to position 300 in SEQ ID NO: 2; Q at a residue corresponding to position 302 in SEQ ID NO: 2; A, P at a residue corresponding to position 303 in SEQ ID NO: 2; S at a residue corresponding to position 304 in SEQ ID NO: 2; V, M, I at a residue corresponding to position 305 in SEQ ID NO: 2; M at a residue corresponding to position 307 in SEQ ID NO: 2; T, A at a residue corresponding to position 311 in SEQ ID NO: 2; F at a residue corresponding to position 313 in SEQ ID NO: 2; Q, H at a residue corresponding to position 314 in SEQ ID NO: 2; A, K, L, R at a residue corresponding to position 315 in SEQ ID NO: 2; I at a residue corresponding to position 316 in SEQ ID NO: 2; D, H at a residue corresponding to position 318 in SEQ ID NO: 2; R, Q at a residue corresponding to position 321 in SEQ ID NO: 2; A, K, H, Q at a residue corresponding to position 322 in SEQ ID NO: 2; F at a residue corresponding to position 330 in SEQ ID NO: 2; S at a residue corresponding to position 333 in SEQ ID NO: 2; V, I at a residue corresponding to position 334 in SEQ ID NO: 2; V, T, M, Y at a residue corresponding to position 336 in SEQ ID NO: 2; L, V at a residue corresponding to position 337 in SEQ ID NO: 2; L, M, V at a residue corresponding to position 338 in SEQ ID NO: 2; T at a residue corresponding to position 339 in SEQ ID NO: 2; G at a residue corresponding to position 340 in SEQ ID NO: 2; I, C, M at a residue corresponding to position 344 in SEQ ID NO: 2; S at a residue corresponding to position 345 in SEQ ID NO: 2; E at a residue corresponding to position 346 in SEQ ID NO: 2; E at a residue corresponding to position 347 in SEQ ID NO: 2; I, L, T at a residue corresponding to position 348 in SEQ ID NO: 2; Y, F at a residue corresponding to position 349 in SEQ ID NO: 2; R, Q at a residue corresponding to position 351 in SEQ ID NO: 2; I at a residue corresponding to position 352 in SEQ ID NO: 2; R at a residue corresponding to position 353 in SEQ ID NO: 2; K at a residue corresponding to position 354 in SEQ ID NO: 2; A at a residue corresponding to position 355 in SEQ ID NO: 2; F, I, V, A, G, L, W at a residue corresponding to position 357 in SEQ ID NO: 2; Q at a residue corresponding to position 358 in SEQ ID NO: 2; Q at a residue corresponding to position 359 in SEQ ID NO: 2; C, H at a residue corresponding to position 362 in SEQ ID NO: 2; V, I at a residue corresponding to position 363 in SEQ ID NO: 2; M at a residue corresponding to position 365 in SEQ ID NO: 2; V, I, L, M at a residue corresponding to position 367 in SEQ ID NO: 2; A at a residue corresponding to position 369 in SEQ ID NO: 2; C at a residue corresponding to position 375 in SEQ ID NO: 2; G, S at a residue corresponding to position 376 in SEQ ID NO: 2; V, C, L at a residue corresponding to position 379 in SEQ ID NO: 2; T, C at a residue corresponding to position 382 in SEQ ID NO: 2; Y at a residue corresponding to position 383 in SEQ ID NO: 2; N at a residue corresponding to position 384 in SEQ ID NO: 2; V, T at a residue corresponding to position 386 in SEQ ID NO: 2; R, H, N, D at a residue corresponding to position 387 in SEQ ID NO: 2; K, H at a residue corresponding to position 388 in SEQ ID NO: 2; A, P, L at a residue corresponding to position 389 in SEQ ID NO: 2; C, I, T, V at a residue corresponding to position 399 in SEQ ID NO: 2; Q at a residue corresponding to position 400 in SEQ ID NO: 2; L, I, C at a residue corresponding to position 401 in SEQ ID NO: 2; V at a residue corresponding to position 404 in SEQ ID NO: 2; P at a residue corresponding to position 407 in SEQ ID NO: 2; G at a residue corresponding to position 409 in SEQ ID NO: 2; R at a residue corresponding to position 416 in SEQ ID NO: 2; Q at a residue corresponding to position 419 in SEQ ID NO: 2; V at a residue corresponding to position 420 in SEQ ID NO: 2; F at a residue corresponding to position 421 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; V at a residue corresponding to position 434 in SEQ ID NO: 2; F at a residue corresponding to position 441 in SEQ ID NO: 2; Y, R, K, C, H at a residue corresponding to position 442 in SEQ ID NO: 2; L, V, M, I at a residue corresponding to position 446 in SEQ ID NO: 2; M at a residue corresponding to position 450 in SEQ ID NO: 2; P at a residue corresponding to position 452 in SEQ ID NO: 2; G at a residue corresponding to position 453 in SEQ ID NO: 2; N, D at a residue corresponding to position 456 in SEQ ID NO: 2; F, I, R at a residue corresponding to position 460 in SEQ ID NO: 2; Y at a residue corresponding to position 461 in SEQ ID NO: 2; L at a residue corresponding to position 465 in SEQ ID NO: 2; A, D at a residue corresponding to position 466 in SEQ ID NO: 2; T at a residue corresponding to position 471 in SEQ ID NO: 2; N, S at a residue corresponding to position 473 in SEQ ID NO: 2; Y, F, V, L, I at a residue corresponding to position 476 in SEQ ID NO: 2; V, P at a residue corresponding to position 479 in SEQ ID NO: 2; S, A, P at a residue corresponding to position 480 in SEQ ID NO: 2; P, E at a residue corresponding to position 481 in SEQ ID NO: 2; L, E, R, A at a residue corresponding to position 485 in SEQ ID NO: 2; C at a residue corresponding to position 490 in SEQ ID NO: 2; P at a residue corresponding to position 492 in SEQ ID NO: 2; V at a residue corresponding to position 507 in SEQ ID NO: 2; V at a residue corresponding to position 511 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; M, L at a residue corresponding to position 526 in SEQ ID NO: 2; L at a residue corresponding to position 527 in SEQ ID NO: 2; R, H at a residue corresponding to position 532 in SEQ ID NO: 2; Q at a residue corresponding to position 540 in SEQ ID NO: 2; Y at a residue corresponding to position 542 in SEQ ID NO: 2; N at a residue corresponding to position 546 in SEQ ID NO: 2; E at a residue corresponding to position 554 in SEQ ID NO: 2; D at a residue corresponding to position 561 in SEQ ID NO: 2; S, T at a residue corresponding to position 564 in SEQ ID NO: 2; T, M, P, V, I at a residue corresponding to position 565 in SEQ ID NO: 2; D at a residue corresponding to position 566 in SEQ ID NO: 2; A, S, Q, N at a residue corresponding to position 567 in SEQ ID NO: 2; G, D, N at a residue corresponding to position 568 in SEQ ID NO: 2; V at a residue corresponding to position 570 in SEQ ID NO: 2; P at a residue corresponding to position 572 in SEQ ID NO: 2; M at a residue corresponding to position 574 in SEQ ID NO: 2; T at a residue corresponding to position 575 in SEQ ID NO: 2; A at a residue corresponding to position 577 in SEQ ID NO: 2; N at a residue corresponding to position 584 in SEQ ID NO: 2; A at a residue corresponding to position 587 in SEQ ID NO: 2; E at a residue corresponding to position 595 in SEQ ID NO: 2; K at a residue corresponding to position 596 in SEQ ID NO: 2; S at a residue corresponding to position 599 in SEQ ID NO: 2; or any combination thereof. It is understood that when alterations are listed herein for a certain position, the alteration can be in the alternative or in the conjunctive depending on the context. For example, the F, L, Y at a residue corresponding to position 69 in SEQ ID NO: 2, means that alterations at a residue corresponding to position 69 in SEQ ID NO: 2 can comprise F or L or Y.

In some embodiments, the recombinant nucleic acid encodes an engineered ACL having: E, R at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; D, N at a residue corresponding to position 10 in SEQ ID NO: 2; A at a residue corresponding to position 12 in SEQ ID NO: 2; Q, P at a residue corresponding to position 14 in SEQ ID NO: 2; S, E at a residue corresponding to position 15 in SEQ ID NO: 2; D at a residue corresponding to position 19 in SEQ ID NO: 2; H, K, R at a residue corresponding to position 20 in SEQ ID NO: 2; E at a residue corresponding to position 22 in SEQ ID NO: 2; V at a residue corresponding to position 41 in SEQ ID NO: 2; I at a residue corresponding to position 45 in SEQ ID NO: 2; R, L, I, V at a residue corresponding to position 50 in SEQ ID NO: 2; F, M, L at a residue corresponding to position 58 in SEQ ID NO: 2; D at a residue corresponding to position 62 in SEQ ID NO: 2; D at a residue corresponding to position 63 in SEQ ID NO: 2; K at a residue corresponding to position 67 in SEQ ID NO: 2; F, L, Y at a residue corresponding to position 69 in SEQ ID NO: 2; A, R at a residue corresponding to position 72 in SEQ ID NO: 2; D, T, A, R at a residue corresponding to position 76 in SEQ ID NO: 2; R at a residue corresponding to position 77 in SEQ ID NO: 2; C at a residue corresponding to position 81 in SEQ ID NO: 2; Q at a residue corresponding to position 86 in SEQ ID NO: 2; R at a residue corresponding to position 87 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; M at a residue corresponding to position 98 in SEQ ID NO: 2; A at a residue corresponding to position 104 in SEQ ID NO: 2; V at a residue corresponding to position 108 in SEQ ID NO: 2; C at a residue corresponding to position 113 in SEQ ID NO: 2; I at a residue corresponding to position 116 in SEQ ID NO: 2; Y, V, F, L at a residue corresponding to position 120 in SEQ ID NO: 2; I at a residue corresponding to position 123 in SEQ ID NO: 2; S at a residue corresponding to position 124 in SEQ ID NO: 2; P, T at a residue corresponding to position 125 in SEQ ID NO: 2; R, A, N at a residue corresponding to position 126 in SEQ ID NO: 2; L at a residue corresponding to position 127 in SEQ ID NO: 2; M at a residue corresponding to position 130 in SEQ ID NO: 2; E at a residue corresponding to position 136 in SEQ ID NO: 2; M, I at a residue corresponding to position 140 in SEQ ID NO: 2; M at a residue corresponding to position 141 in SEQ ID NO: 2; Q, N at a residue corresponding to position 142 in SEQ ID NO: 2; R at a residue corresponding to position 145 in SEQ ID NO: 2; S at a residue corresponding to position 147 in SEQ ID NO: 2; V at a residue corresponding to position 148 in SEQ ID NO: 2; V at a residue corresponding to position 149 in SEQ ID NO: 2; C at a residue corresponding to position 151 in SEQ ID NO: 2; E at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 190 in SEQ ID NO: 2; E, A at a residue corresponding to position 191 in SEQ ID NO: 2; A at a residue corresponding to position 193 in SEQ ID NO: 2; S at a residue corresponding to position 195 in SEQ ID NO: 2; L, A at a residue corresponding to position 201 in SEQ ID NO: 2; T at a residue corresponding to position 205 in SEQ ID NO: 2; I at a residue corresponding to position 209 in SEQ ID NO: 2; Y at a residue corresponding to position 214 in SEQ ID NO: 2; T at a residue corresponding to position 219 in SEQ ID NO: 2; G at a residue corresponding to position 220 in SEQ ID NO: 2; K at a residue corresponding to position 221 in SEQ ID NO: 2; S at a residue corresponding to position 228 in SEQ ID NO: 2; H, K at a residue corresponding to position 230 in SEQ ID NO: 2; L at a residue corresponding to position 232 in SEQ ID NO: 2; S at a residue corresponding to position 234 in SEQ ID NO: 2; A at a residue corresponding to position 237 in SEQ ID NO: 2; M at a residue corresponding to position 238 in SEQ ID NO: 2; V at a residue corresponding to position 239 in SEQ ID NO: 2; Q, E at a residue corresponding to position 241 in SEQ ID NO: 2; C, V, T at a residue corresponding to position 242 in SEQ ID NO: 2; W, F, M at a residue corresponding to position 243 in SEQ ID NO: 2; P, Q at a residue corresponding to position 244 in SEQ ID NO: 2; I at a residue corresponding to position 246 in SEQ ID NO: 2; R at a residue corresponding to position 247 in SEQ ID NO: 2; L at a residue corresponding to position 253 in SEQ ID NO: 2; M at a residue corresponding to position 254 in SEQ ID NO: 2; S, H at a residue corresponding to position 260 in SEQ ID NO: 2; Y at a residue corresponding to position 263 in SEQ ID NO: 2; N at a residue corresponding to position 266 in SEQ ID NO: 2; M, F at a residue corresponding to position 267 in SEQ ID NO: 2; A at a residue corresponding to position 272 in SEQ ID NO: 2; I at a residue corresponding to position 273 in SEQ ID NO: 2; C at a residue corresponding to position 274 in SEQ ID NO: 2; T, A at a residue corresponding to position 278 in SEQ ID NO: 2; M at a residue corresponding to position 279 in SEQ ID NO: 2; V at a residue corresponding to position 281 in SEQ ID NO: 2; E at a residue corresponding to position 283 in SEQ ID NO: 2; M, I, L, V at a residue corresponding to position 291 in SEQ ID NO: 2; H at a residue corresponding to position 292 in SEQ ID NO: 2; V, I, M at a residue corresponding to position 295 in SEQ ID NO: 2; M, V at a residue corresponding to position 298 in SEQ ID NO: 2; Q at a residue corresponding to position 300 in SEQ ID NO: 2; Q at a residue corresponding to position 302 in SEQ ID NO: 2; A, P at a residue corresponding to position 303 in SEQ ID NO: 2; S at a residue corresponding to position 304 in SEQ ID NO: 2; V at a residue corresponding to position 305 in SEQ ID NO: 2; M at a residue corresponding to position 307 in SEQ ID NO: 2; T, A at a residue corresponding to position 311 in SEQ ID NO: 2; F at a residue corresponding to position 313 in SEQ ID NO: 2; Q, H at a residue corresponding to position 314 in SEQ ID NO: 2; A, K at a residue corresponding to position 315 in SEQ ID NO: 2; D at a residue corresponding to position 318 in SEQ ID NO: 2; R, Q at a residue corresponding to position 321 in SEQ ID NO: 2; A, K, H, Q at a residue corresponding to position 322 in SEQ ID NO: 2; F at a residue corresponding to position 330 in SEQ ID NO: 2; S at a residue corresponding to position 333 in SEQ ID NO: 2; V, I at a residue corresponding to position 334 in SEQ ID NO: 2; V, T, M at a residue corresponding to position 336 in SEQ ID NO: 2; L, V at a residue corresponding to position 337 in SEQ ID NO: 2; L, M at a residue corresponding to position 338 in SEQ ID NO: 2; G at a residue corresponding to position 340 in SEQ ID NO: 2; I at a residue corresponding to position 344 in SEQ ID NO: 2; I, L at a residue corresponding to position 348 in SEQ ID NO: 2; Y at a residue corresponding to position 349 in SEQ ID NO: 2; R, Q at a residue corresponding to position 351 in SEQ ID NO: 2; R at a residue corresponding to position 353 in SEQ ID NO: 2; K at a residue corresponding to position 354 in SEQ ID NO: 2; A at a residue corresponding to position 355 in SEQ ID NO: 2; F, I at a residue corresponding to position 357 in SEQ ID NO: 2; Q at a residue corresponding to position 358 in SEQ ID NO: 2; Q at a residue corresponding to position 359 in SEQ ID NO: 2; C, H at a residue corresponding to position 362 in SEQ ID NO: 2; V at a residue corresponding to position 363 in SEQ ID NO: 2; M at a residue corresponding to position 365 in SEQ ID NO: 2; V, I, L, M at a residue corresponding to position 367 in SEQ ID NO: 2; A at a residue corresponding to position 369 in SEQ ID NO: 2; G at a residue corresponding to position 376 in SEQ ID NO: 2; V, C at a residue corresponding to position 379 in SEQ ID NO: 2; T, C at a residue corresponding to position 382 in SEQ ID NO: 2; Y at a residue corresponding to position 383 in SEQ ID NO: 2; V, T at a residue corresponding to position 386 in SEQ ID NO: 2; R, H, N at a residue corresponding to position 387 in SEQ ID NO: 2; K at a residue corresponding to position 388 in SEQ ID NO: 2; A, P at a residue corresponding to position 389 in SEQ ID NO: 2; C, I, T, V at a residue corresponding to position 399 in SEQ ID NO: 2; Q at a residue corresponding to position 400 in SEQ ID NO: 2; L, I at a residue corresponding to position 401 in SEQ ID NO: 2; V at a residue corresponding to position 404 in SEQ ID NO: 2; P at a residue corresponding to position 407 in SEQ ID NO: 2; G at a residue corresponding to position 409 in SEQ ID NO: 2; V at a residue corresponding to position 420 in SEQ ID NO: 2; F at a residue corresponding to position 421 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; V at a residue corresponding to position 434 in SEQ ID NO: 2; Y, R, K, C, H at a residue corresponding to position 442 in SEQ ID NO: 2; L, V, M, I at a residue corresponding to position 446 in SEQ ID NO: 2; M at a residue corresponding to position 450 in SEQ ID NO: 2; P at a residue corresponding to position 452 in SEQ ID NO: 2; G at a residue corresponding to position 453 in SEQ ID NO: 2; N, D at a residue corresponding to position 456 in SEQ ID NO: 2; F, I at a residue corresponding to position 460 in SEQ ID NO: 2; Y at a residue corresponding to position 461 in SEQ ID NO: 2; L at a residue corresponding to position 465 in SEQ ID NO: 2; A at a residue corresponding to position 466 in SEQ ID NO: 2; N, S at a residue corresponding to position 473 in SEQ ID NO: 2; Y, F, V, L, I at a residue corresponding to position 476 in SEQ ID NO: 2; V, P at a residue corresponding to position 479 in SEQ ID NO: 2; S, A at a residue corresponding to position 480 in SEQ ID NO: 2; P, E at a residue corresponding to position 481 in SEQ ID NO: 2; L, E, R, A at a residue corresponding to position 485 in SEQ ID NO: 2; P at a residue corresponding to position 492 in SEQ ID NO: 2; V at a residue corresponding to position 507 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P at a residue corresponding to position 521 in SEQ ID NO: 2; M, L at a residue corresponding to position 526 in SEQ ID NO: 2; L at a residue corresponding to position 527 in SEQ ID NO: 2; R, H at a residue corresponding to position 532 in SEQ ID NO: 2; Q at a residue corresponding to position 540 in SEQ ID NO: 2; Y at a residue corresponding to position 542 in SEQ ID NO: 2; E at a residue corresponding to position 554 in SEQ ID NO: 2; S, T at a residue corresponding to position 564 in SEQ ID NO: 2; T, M, P, V, I at a residue corresponding to position 565 in SEQ ID NO: 2; A, S, Q, N at a residue corresponding to position 567 in SEQ ID NO: 2; G, D at a residue corresponding to position 568 in SEQ ID NO: 2; V at a residue corresponding to position 570 in SEQ ID NO: 2; P at a residue corresponding to position 572 in SEQ ID NO: 2; T at a residue corresponding to position 575 in SEQ ID NO: 2; N at a residue corresponding to position 584 in SEQ ID NO: 2; A at a residue corresponding to position 587 in SEQ ID NO: 2; E at a residue corresponding to position 595 in SEQ ID NO: 2; K at a residue corresponding to position 596 in SEQ ID NO: 2; or any combination thereof. It is understood that when alterations are listed herein for a certain position, the alteration can be in the alternative or in the conjunctive depending on the context. For example, the F, L, Y at a residue corresponding to position 69 in SEQ ID NO: 2, means that alterations at a residue corresponding to position 69 in SEQ ID NO: 2 can comprise F or L or Y.

In some embodiments, the recombinant nucleic acid encodes an engineered ACL having one or more amino acid alterations comprising: D5R, D5E, A6L, S7K, S7T, G8P, E10K, E10D, E10P, E10N, E10R, L12A, L12I, L12V, L12M, P13C, H14P, H14A, H14Q, D15E, D15L, D15S, D15R, V16C, V16L, V16I, E19D, E19R, S20H, S20K, S20R, S20D, P22D, P22E, S25N, L28M, V37C, P39R, N40T, T41M, T41L, T41V, T41I, A43S, W44M, L45I, L45V, W48P, K50I, K50F, K50R, K50V, K50L, E51R, P53L, A57F, I58V, I58L, I58M, I58F, A59V, R61P, R61E, A62D, G63D, W66G, R67K, H69F, H69Y, H69L, Y71F, Y71W, N72A, N72R, L74F, L75D, N76T, N76D, N76A, N76R, M77R, V78A, N80K, V81C, G83S, G83H, G83Q, G83N, A84Y, A84H, L85F, L86A, L86Q, A87R, R88L, A92K, D93G, S94R, P95R, V97A, V98M, M99L, S100C, G101P, G103R, V104L, V104W, V104A, H106W, H106F, A107V, A107P, A107I, A107L, A107W, A107F, I108V, L109V, S110M, S110F, S110Y, S110A, S110D, A113T, A113C, A113V, Q114M, Q114W, Q114L, Q114G, Q114A, Y115A, V116I, P119V, P119T, P119I, I120M, I120F, I120V, I120A, I120L, I120W, I120Y, V121T, P122A, L123M, L123V, L123I, A124Y, A124S, A124N, E125P, E125T, E125W, Q126M, Q126N, Q126R, Q126A, Y127F, Y127W, Y127L, S128R, I130M, P131E, R136E, R136Q, R136K, L137M, V140M, V140C, V140I, L141M, E142N, E142Q, K145G, K145R, K145D, A147S, M148L, M148V, M148A, A149V, A149L, F150V, V151C, Y157L, S159R, S159E, L161I, L163V, P164A, Q165R, E167R, G168H, V169I, E170I, E170V, E170L, A173V, K175R, T176D, D177E, V181E, V183I, A185S, F186Y, A187D, L189V, L190V, G191A, G191E, H192T, G193A, Q194S, G195S, G196P, H201A, H201P, H201L, G205T, T208D, L209I, L209T, L209P, L209V, K211L, K211T, K211C, K211M, K211V, K211A, I212L, I212M, L213I, L213C, L213V, L213Q, L213M, F214Y, T215S, S216G, S218T, S219T, S220G, D221T, D221M, D221L, D221R, D221K, D221H, P222S, G224A, V225A, L226M, L226I, L226V, T227L, T227D, T227H, T227I, T228S, R230H, R230K, M231N, M232L, M232I, M232V, C233L, C233V, C233M, C233W, C233A, C233I, A234S, A234W, N235Q, N235S, Q236A, Q236C, Q236I, Q236M, Q236V, T237A, T237M, T237Q, T237R, T237E, Q238M, Q238S, Q238W, Q238N, Q238H, 1239F, 1239M, 1239V, 1239C, 1239L, S240A, S240R, D241A, D241H, D241E, D241Q, A242V, A242C, A242T, A242W, L243I, L243F, L243M, L243W, L243V, R244E, R244P, R244Q, R244H, R244G, F245M, F245L, F245H, F245W, F245Y, L246I, L246T, L246M, K247R, K247P, K247E, P251E, R252H, R252V, I253Y, I253M, I253F, I253L, I253V, L254I, L254F, L254V, L254M, D255A, D255T, D255N, D255L, D255I, D255G, D255H, D255C, D255V, D255S, W256F, W256V, W256I, W256C, W256Y, L257M, W259L, W259F, N260H, N260S, N260Y, N260F, V262C, V262T, V262A, V262I, V262M, F263A, F263Y, F263H, G264A, G265M, G265L, G265W, G265F, G265C, S266N, S266T, S266M, S266V, H267I, H267G, H267M, H267W, H267F, H267L, H267V, H267C, H267A, N268M, N268A, N268C, N268G, N268V, F269L, F269M, F269I, F269V, N270M, N270H, N270Y, N270T, M271A, M271P, M271C, M271T, M271L, M272C, M272V, M272P, M272A, M272T, L273M, L273V, L273F, L273I, A274Y, A274R, A274V, A274M, A274C, A274H, A274W, N275G, N275C, N275S, N275H, G277A, G277C, S278C, S278T, S278A, S278H, L279V, L279M, L279I, I281F, I281L, I281V, I281M, D282P, D282M, D282E, D282A, D282H, N283E, N283D, N283P, N283R, G284F, G284R, G284W, K285R, K285F, K285W, K285D, P286D, P286F, T287P, T287A, T287V, G290A, F291M, F291V, F291L, F291P, F291I, E292H, T294I, T294L, T294A, L295W, L295M, L295V, L295I, R296D, R296Q, I298M, I298V, I298L, K299A, E300Q, P302Q, G303V, G303I, G303A, G303P, G303C, T304S, L305W, L305Y, L305V, L305I, L305F, L305H, L305M, A306V, A306Y, A306I, A306M, A306L, A306F, F307P, F307W, F307Y, F307H, F307M, N308T, N308A, N308S, N308L, N308G, N308V, N308M, V311A, V311R, V311P, V311T, G312I, G312M, G312V, G312F, Y313L, Y313F, Y313W, A314Q, A314E, A314H, M315R, M315A, M315K, M315L, L316W, L316M, L316I, L316F, N318H, N318D, E321R, E321Q, R322Q, R322K, R322A, R322H, Y330L, Y330F, F331G, E333S, L334I, L334M, L334V, D335R, D335K, L336T, L336I, L336M, L336Y, L336V, L336F, L336W, I337L, I337M, I337F, I337A, I337V, F338W, F338L, F338M, F338V, Y339F, Y339T, Y339S, Y339C, A340G, G341A, G341S, A342S, S343P, S343A, L344M, L344I, L344V, L344C, P345S, Q346V, Q346A, Q346P, Q346E, D347E, V348T, V348I, V348L, W349M, W349F, W349L, W349A, W3491, W349Y, S351F, S351R, S351Q, S351W, L352I, L352M, L352Y, E353R, R354K, F355A, F355R, M357W, M357G, M357V, M357L, M357P, M357I, M357F, M357A, E358Q, E359H, E359Q, G361F, S362H, S362L, S362C, L363D, L363V, L363I, L363G, L365M, L365V, L365I, M366I, M366V, M366L, M366F, A367T, A367C, A367M, A367I, A367V, A367L, S368N, S368Q, S368E, S368T, S369G, S369A, W370Y, W370F, M372L, M372Q, T373S, T375A, T375C, A376T, A376S, A376C, A376G, P377G, P377M, A378C, A378T, A378V, T379C, T379V, T379I, T379L, T379A, I380F, I380C, I380H, I380S, I380M, I380T, I380V, M381N, M381F, M381L, M381C, M381V, V382L, V382N, V382C, V382T, V382P, H383D, H383N, H383R, H383P, H383Y, E384P, E384N, E384D, E384W, P385G, P385D, I386D, I386V, I386L, I386T, G387D, G387R, G387N, G387H, G387E, R388K, R388H, S389A, S389P, S389L, V391S, V391T, I392V, I392C, V394R, V394K, V394I, V394Q, V394L, P395A, L396F, L396I, L396M, L396V, G398D, G398H, G398N, A399M, A399V, A399T, A399C, A399I, E400D, E400Q, V401M, V401I, V401C, V401L, V401A, K402R, L404V, P405D, D406E, D407P, M409Q, M409E, M409G, C411D, C411S, C411V, C411G, C411N, L413I, L413V, R414W, R414L, R414V, R414C, R414Q, V415I, K416R, G417S, P418D, N419H, N419S, N419Q, N419M, I420V, M421T, M421F, F425H, F425L, K430A, A432K, E433R, E433D, A434T, A434V, F435I, F435M, F435L, L441F, L441M, L441Y, V442K, V442H, V442Y, V442R, V442C, T443S, A446M, A446I, A446L, A446V, V447A, V447G, F449I, V450D, V450M, D451E, D452P, A453G, P455E, P455G, P455R, S456D, S456E, S456Q, S456N, G458N, G458F, G458Y, G458W, L459Y, V460Y, V460F, V460I, V460R, V460W, F461I, F461L, F461Y, D462I, D462T, D462V, G463D, R464D, V465L, S466D, S466K, S466A, E467K, D468M, F469L, K470V, L471T, L471V, T473S, T473N, G474E, W476N, W476Y, W476L, W476F, W476V, W476I, Q478T, Q478L, A479P, A479V, G480L, G480I, G480S, G480P, G480A, G480V, V481P, V481E, R483E, D485A, D485L, D485E, D485R, L490C, G492P, L493F, D496H, V497A, V498C, H502P, R504H, I507Y, I507V, I507W, G508V, F510A, 1511V, I511L, F512I, P513V, L514N, P515D, P515S, G516A, V518I, S519T, T521P, T521W, V526L, V526M, I527L, S532R, S532H, I535L, R540Q, M542Y, M542W, A546N, R552Q, N554E, I558F, S560D, E561D, P563F, P563L, P563W, Q564D, Q564K, Q564S, Q564L, Q564T, Q564R, L565M, L565I, L565V, L565P, L565T, K566A, K566E, K566D, K566P, D567K, D567Q, D567T, D567N, D567S, D567A, H568G, H568N, H568D, E569M, E569K, I570V, I570L, D572R, D572P, K573T, K573S, G574M, G574L, S575A, S575Y, S575K, S575T, N577Y, N577A, N577L, I578Q, I578A, T583D, R584N, R585H, R585L, D587A, L588Q, R591Q, R591E, Y593A, N595E, E596K, P598D, A599S, I601L or any combination thereof, in reference to SEQ ID NO: 2.

In some embodiments, the recombinant nucleic acid encodes an engineered ACL having one or more amino acid alterations comprising: D5E, D5R, A6L, S7K, S7T, G8P, E10D, E10N, E10R, E10P, E10K, L12A, L12V, L12M, L12I, P13C, H14Q, H14P, H14A, D15S, D15E, V161, V16C, E19D, E19R, S20H, S20K, S20R, S20D, P22E, P22D, S25N, L28M, V37C, P39R, N40T, T41V, T41I, T41M, A43S, L45I, L45V, K50R, K50L, K50I, K50V, K50F, E51R, P53L, A57F, I58F, I58M, I58L, I58V, A59V, R61P, A62D, G63D, R67K, H69F, H69L, H69Y, Y71W, Y71F, N72A, N72R, L74F, L75D, N76D, N76T, N76A, N76R, M77R, V78A, N80K, V81C, G83S, G83N, A84Y, A84H, L85F, L86Q, L86A, A87R, R88L, A92K, D93G, S94R, P95R, V97A, V98M, M99L, S100C, G101P, G103R, V104A, V104L, V104W, H106F, I108V, L109V, S110A, S110M, A113C, V116I, P119T, P119V, P119I, I120Y, I120V, I120F, I120L, I120M, I120A, V121T, L123I, L123M, L123V, A124S, A124N, A124Y, E125P, E125T, E125W, Q126R, Q126A, Q126N, Q126M, Y127L, Y127W, Y127F, S128R, I130M, P131E, R136E, R136K, R136Q, L137M, V140M, V140I, V140C, L141M, E142Q, E142N, K145R, K145D, K145G, A147S, M148V, M148L, M148A, A149V, F150V, V151C, S159E, S159R, L161I, L163V, P164A, Q165R, E167R, G168H, V169I, E170I, E170L, E170V, A173V, K175R, T176D, D177E, V181E, V183I, A185S, F186Y, A187D, L189V, L190V, G191E, G191A, H192T, G193A, Q194S, G195S, G196P, H201L, H201A, G205T, T208D, L209I, L209T, L209V, K211L, I212L, I212M, L213I, L213M, L213V, F214Y, T215S, S218T, S219T, S220G, D221K, D221L, D221T, D221H, D221R, D221M, P222S, G224A, L226M, L226V, L226I, T227I, T228S, R230H, R230K, M232L, C233A, A234S, N235Q, Q236M, T237A, T237M, T237Q, Q238M, Q238H, I239V, I239L, I239M, S240A, D241Q, D241E, D241H, D241A, A242C, A242V, A242T, L243W, L243F, L243M, L243I, L243V, R244P, R244Q, R244G, R244H, R244E, F245L, F245M, F245W, L246I, K247R, K247P, R252V, R252H, I253L, I253V, I253M, L254M, L254I, L254V, D255C, D255V, D255T, D255N, W256Y, W256V, W256F, N260S, N260H, N260F, N260Y, V262T, V262C, V262A, F263Y, G264A, G265M, S266N, S266M, H267M, H267F, H267L, H267W, H267G, N268M, N268A, N270M, M271L, M271C, M271A, M272A, M272V, M272T, M272P, M272C, L273I, L273V, L273M, L273F, A274C, A274M, A274H, A274R, A274Y, N275H, N275C, G277A, S278T, S278A, S278C, L279M, L279I, L279V, I281V, I281L, N283E, N283D, N283P, N283R, K285R, K285F, K285D, P286D, P286F, T287A, T287P, T287V, G290A, F291M, F291I, F291L, F291V, E292H, T294A, L295V, L295I, L295M, R296Q, R296D, I298M, I298V, I298L, K299A, E300Q, P302Q, G303A, G303P, G303C, G303V, T304S, L305V, L305M, L305I, A306I, A306V, A306Y, F307M, F307W, F307P, N308G, V311T, V311A, V311R, G312F, G312V, G312M, Y313F, Y313W, A314Q, A314H, A314E, M315A, M315K, M315L, M315R, L316I, L316M, N318D, N318H, E321R, E321Q, R322A, R322K, R322H, R322Q, Y330F, F331G, E333S, L334V, L334I, L334M, D335K, L336V, L336T, L336M, L336Y, L336W, L336I, L336F, I337L, I337V, I337M, F338L, F338M, F338V, F338W, Y339T, Y339S, Y339F, A340G, A342S, S343P, S343A, L344I, L344C, L344M, L344V, P345S, Q346E, Q346V, Q346A, Q346P, D347E, V348I, V348L, V348T, W349Y, W349F, W349L, S351R, S351Q, S351W, L352I, L352M, E353R, R354K, F355A, M357F, M357I, M357V, M357A, M357G, M357L, M357W, E358Q, E359Q, S362C, S362H, S362L, L363V, L363I, L363G, L365M, L365I, L365V, M366I, M366L, M366F, M366V, A367V, A367I, A367L, A367M, A367T, A367C, S368N, S369A, S369G, W370Y, T375C, T375A, A376G, A376S, P377G, A378T, T379V, T379C, T379L, T379A, T379I, I380S, I380V, I380H, I380T, M381L, M381C, V382T, V382C, V382N, V382L, V382P, H383Y, H383R, E384N, E384W, E384D, P385G, I386V, I386T, I386L, G387R, G387H, G387N, G387D, G387E, R388K, R388H, S389A, S389P, S389L, V391S, V391T, I392V, V394I, V394K, L396V, G398H, A399C, A399I, A399T, A399V, E400Q, E400D, V401L, V401I, V401C, V401A, V401M, L404V, D406E, D407P, M409G, M409Q, M409E, C411S, R414L, R414C, K416R, N419Q, N419H, N419M, N419S, I420V, M421F, M421T, F425L, F425H, K430A, A432K, E433R, E433D, A434V, A434T, F435M, L441F, L441M, V442Y, V442R, V442K, V442C, V442H, T443S, A446L, A446V, A446M, A446I, V447A, V450M, V450D, D451E, D452P, A453G, S456N, S456D, V460F, V460I, V460R, V460W, V460Y, F461Y, F461L, D462V, D462I, G463D, V465L, S466A, S466D, S466K, E467K, D468M, F469L, K470V, L471T, L471V, T473N, T473S, W476Y, W476F, W476V, W476L, W476I, W476N, Q478T, A479V, A479P, G480S, G480A, G480P, G480L, V481P, V481E, D485L, D485E, D485R, D485A, L490C, G492P, V498C, I507V, G508V, F510A, I511V, I511L, F512I, L514N, P515D, P515S, G516A, V518I, S519T, T521P, T521W, V526M, V526L, I527L, S532R, S532H, I535L, R540Q, M542Y, M542W, A546N, R552Q, N554E, S560D, E561D, P563L, P563F, Q564S, Q564T, Q564L, Q564D, Q564R, Q564K, L565T, L565M, L565P, L565V, L565I, K566D, K566A, K566E, K566P, D567A, D567S, D567Q, D567N, D567T, D567K, H568G, H568D, H568N, E569M, E569K, I570V, I570L, D572P, K573S, K573T, G574M, G574L, S575T, S575Y, N577A, N577L, I578A, I578Q, T583D, R584N, R585H, D587A, L588Q, R591Q, R591E, Y593A, N595E, E596K, P598D, A599S, or I601L, in reference to SEQ ID NO: 2.

In some embodiments, the recombinant nucleic acid encodes an engineered ACL having one or more amino acid alterations comprising: D5E, D5R, A6L, S7K, S7T, G8P, E10D, E10N, E10R, E10P, L12A, L12V, L12M, H14Q, H14P, D15S, D15E, V16I, E19D, E19R, S20H, S20K, S20R, S20D, P22E, P22D, L28M, P39R, T41V, A43S, L45I, K50R, K50L, K50I, K50V, K50F, A57F, I58F, I58M, I58L, I58V, R61P, A62D, G63D, R67K, H69F, H69L, H69Y, Y71W, Y71F, N72A, N72R, N76D, N76T, N76A, N76R, M77R, V78A, V81C, A84Y, L85F, L86Q, A87R, A92K, D93G, S94R, P95R, V98M, V104A, I108V, L109V, A113C, V116I, P119T, I120Y, I120V, I120F, I120L, I120M, I120A, L123I, A124S, A124N, E125P, E125T, E125W, Q126R, Q126A, Q126N, Y127L, Y127W, I130M, P131E, R136E, R136K, V140M, V140I, V140C, L141M, E142Q, E142N, K145R, A147S, M148V, A149V, V151C, S159E, S159R, L161I, L163V, P164A, E167R, G168H, V169I, E170I, E170L, E170V, A173V, K175R, T176D, D177E, V181E, V183I, A185S, F186Y, A187D, L190V, G191E, G191A, G193A, Q194S, G195S, H201L, H201A, G205T, L209I, F214Y, S218T, S219T, S220G, D221K, D221L, D221T, D221H, L226M, L226V, T228S, R230H, R230K, M232L, A234S, T237A, Q238M, I239V, D241Q, D241E, A242C, A242V, A242T, L243W, L243F, L243M, R244P, R244Q, L246I, K247R, I253L, I253V, L254M, L254I, L254V, N260S, N260H, V262T, F263Y, S266N, H267M, H267F, M272A, L273I, L273V, L273M, L273F, A274C, S278T, S278A, L279M, I281V, N283E, K285R, G290A, F291M, F291I, F291L, F291V, E292H, L295V, L295I, L295M, R296Q, I298M, I298V, E300Q, P302Q, G303A, G303P, T304S, L305V, L305M, L305I, F307M, V311T, V311A, Y313F, A314Q, A314H, M315A, M315K, M315L, M315R, L316I, N318D, N318H, E321R, E321Q, R322A, R322K, R322H, R322Q, Y330F, E333S, L334V, L334I, L336V, L336T, L336M, L336Y, I337L, I337V, F338L, F338M, F338V, Y339T, A340G, L344I, L344C, L344M, P345S, Q346E, D347E, V348I, V348L, V348T, W349Y, W349F, S351R, S351Q, L352I, E353R, R354K, F355A, M357F, M357I, M357V, M357A, M357G, M357L, M357W, E358Q, E359Q, S362C, S362H, L363V, L363I, L365M, A367V, A367I, A367L, A367M, S369A, T375C, A376G, A376S, T379V, T379C, T379L, V382T, V382C, H383Y, E384N, I386V, I386T, G387R, G387H, G387N, G387D, R388K, R388H, S389A, S389P, S389L, A399C, A399I, A399T, A399V, E400Q, V401L, V401I, V401C, L404V, D407P, M409G, K416R, N419Q, I420V, M421F, K430A, A432K, E433R, E433D, A434V, L441F, V442Y, V442R, V442K, V442C, V442H, A446L, A446V, A446M, A446I, V450M, D452P, A453G, S456N, S456D, V460F, V460I, V460R, F461Y, V465L, S466A, S466D, L471T, T473N, T473S, W476Y, W476F, W476V, W476L, W476I, A479V, A479P, G480S, G480A, G480P, V481P, V481E, D485L, D485E, D485R, D485A, L490C, G492P, I507V, I511V, L514N, P515D, P515S, G516A, V518I, S519T, T521P, T521W, V526M, V526L, I527L, S532R, S532H, R540Q, M542Y, A546N, N554E, E561D, Q564S, Q564T, L565T, L565M, L565P, L565V, L565I, K566D, D567A, D567S, D567Q, D567N, H568G, H568D, H568N, I570V, D572P, G574M, S575T, N577A, R584N, D587A, N595E, E596K, or A599S, in reference to SEQ ID NO: 2.

In some embodiments, the recombinant nucleic acid encodes an engineered ACL having one or more amino acid alterations comprising: D5E, D5R, A6L, S7K, S7T, G8P, E10D, E10N, L12A, H14Q, H14P, D15S, D15E, E19D, S20H, S20K, S20R, P22E, T41V, L45I, K50R, K50L, K50I, K50V, I58F, I58M, I58L, A62D, G63D, R67K, H69F, H69L, H69Y, N72A, N72R, N76D, N76T, N76A, N76R, M77R, V81C, L86Q, A87R, A92K, D93G, S94R, P95R, V98M, V104A, I108V, A113C, V116I, I120Y, I120V, I120F, I120L, L123I, A124S, E125P, E125T, Q126R, Q126A, Q126N, Y127L, I130M, R136E, V140M, V140I, L141M, E142Q, E142N, K145R, A147S, M148V, A149V, V151C, S159E, L161I, L163V, P164A, E167R, G168H, V169I, K175R, T176D, D177E, A185S, F186Y, A187D, L190V, G191E, G191A, G193A, G195S, H201L, H201A, G205T, L209I, F214Y, S219T, S220G, D221K, T228S, R230H, R230K, M232L, A234S, T237A, Q238M, I239V, D241Q, D241E, A242C, A242V, A242T, L243W, L243F, L243M, R244P, R244Q, L246I, K247R, I253L, L254M, N260S, N260H, F263Y, S266N, H267M, H267F, M272A, L273I, A274C, S278T, S278A, L279M, I281V, N283E, F291M, F291I, F291L, F291V, E292H, L295V, L295I, L295M, I298M, I298V, E300Q, P302Q, G303A, G303P, T304S, L305V, F307M, V311T, V311A, Y313F, A314Q, A314H, M315A, M315K, N318D, E321R, E321Q, R322A, R322K, R322H, R322Q, Y330F, E333S, L334V, L334I, L336V, L336T, L336M, I337L, I337V, F338L, F338M, A340G, L344I, V348I, V348L, W349Y, S351R, S351Q, E353R, R354K, F355A, M357F, M357I, E358Q, E359Q, S362C, S362H, L363V, L365M, A367V, A367I, A367L, A367M, S369A, A376G, T379V, T379C, V382T, V382C, H383Y, I386V, I386T, G387R, G387H, G387N, R388K, S389A, S389P, A399C, A399I, A399T, A399V, E400Q, V401L, V401I, L404V, D407P, M409G, I420V, M421F, K430A, A432K, E433R, E433D, A434V, V442Y, V442R, V442K, V442C, V442H, A446L, A446V, A446M, A446I, V450M, D452P, A453G, S456N, S456D, V460F, V460I, F461Y, V465L, S466A, T473N, T473S, W476Y, W476F, W476V, W476L, W476I, A479V, A479P, G480S, G480A, V481P, V481E, D485L, D485E, D485R, D485A, G492P, I507V, L514N, P515D, P515S, G516A, V518I, S519T, T521P, V526M, V526L, I527L, S532R, S532H, R540Q, M542Y, N554E, Q564S, Q564T, L565T, L565M, L565P, L565V, L565I, D567A, D567S, D567Q, D567N, H568G, H568D, I570V, D572P, S575T, R584N, D587A, N595E, or E596K, in reference to SEQ ID NO: 2.

In some embodiments, provided herein is a recombinant nucleic acid that hybridizes under highly stringent hybridization conditions to an isolated nucleic acid encoding an engineered ACL described herein. Accordingly, in some embodiments, the recombinant nucleic acid is an isolated nucleic acid that hybridizes under highly stringent hybridization conditions to a nucleic acid that encodes an engineered ACL that is a variant of a wild-type ACL (SEQ ID NO: 1 or 2), such as an engineered ACL having one or more alterations at a position described in TABLE 8 or TABLE 9. In some embodiments, the recombinant nucleic acid molecule is an isolated nucleic acid that hybridizes under highly stringent hybridization conditions to a nucleic acid that encodes an engineered ACL having one or more alterations at a position described in TABLE 8. In some embodiments, the recombinant nucleic acid is an isolated nucleic acid that hybridizes under highly stringent hybridization conditions to a nucleic acid that encodes an engineered ACL having a combination of alterations described in TABLE 9.

In some embodiments, provided herein is a recombinant nucleic acid encoding an engineered ACL provided herein has an amino acid sequence that is a variant of SEQ ID NO: 1 that includes one or more alterations as described in TABLE 8, wherein the portion, other than the one or more alterations described in TABLE 8, of the engineered ACL has at least 65%, 70%, 75%, 80%, 85%, 90%, 95%, 96%, 97%, 98% or 99% sequence identity, or is identical, to an amino acid sequence referenced as SEQ ID NO: 1. Accordingly, in some embodiments, a recombinant nucleic acid encodes an engineered ACL that has an amino acid sequence that includes one or more alterations as described in TABLE 8, other than the alteration described in TABLE 8, of the engineered ACL has at least 65% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL has at least 70% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL has at least 75% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL has at least 80% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL has at least 85% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL has at least 90% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL has at least 95% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL has at least 96% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL has at least 97% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL has at least 98% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL has at least 99% sequence identity to an amino acid sequence referenced as SEQ ID NO: 1. In other embodiments, a recombinant nucleic acid encodes an engineered ACL that has an amino acid sequence that includes one or more alterations as described in TABLE 8, other than the alteration described in TABLE 8, of the engineered ACL is identical to an amino acid sequence referenced as SEQ ID NO: 1.

In some embodiments, provided herein is a recombinant nucleic acid encoding an engineered ACL provided herein has an amino acid sequence that is a variant of SEQ ID NO: 2 that includes one or more alterations as described in TABLE 9, wherein the portion, other than the one or more alterations described in TABLE 9, of the engineered ACL has at least 65%, 70%, 75%, 80%, 85%, 90%, 95%, 96%, 97%, 98% or 99% sequence identity, or is identical, to an amino acid sequence referenced as SEQ ID NO: 2. Accordingly, in some embodiments, a recombinant nucleic acid encodes an engineered ACL that has an amino acid sequence that includes one or more alterations as described in TABLE 9, other than the alteration described in TABLE 9, of the engineered ACL has at least 65% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL has at least 70% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL has at least 75% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL has at least 80% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL has at least 85% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL has at least 90% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL has at least 95% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL has at least 96% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL has at least 97% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL has at least 98% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL has at least 99% sequence identity to an amino acid sequence referenced as SEQ ID NO: 2. In other embodiments, a recombinant nucleic acid encodes an engineered ACL that has an amino acid sequence that includes one or more alterations as described in TABLE 9, other than the alteration described in TABLE 9, of the engineered ACL is identical to an amino acid sequence referenced as SEQ ID NO: 2.

In some embodiments, provided herein is a recombinant nucleic acid that hybridizes under highly stringent hybridization conditions to an isolated nucleic acid encoding an engineered ACL described herein. Accordingly, in some embodiments, the recombinant nucleic acid is an isolated nucleic acid that hybridizes under highly stringent hybridization conditions to a nucleic acid that encodes an engineered ACL that is a variant of a wild-type or parent ACL (SEQ ID NO: 1), such as an engineered ACL having one or more alterations at a position described in TABLE 8, and, in some embodiments, a one or more, or a combination of alternations described in TABLE 9. In some embodiments, the recombinant nucleic acid molecule is an isolated nucleic acid that hybridizes under highly stringent hybridization conditions to a nucleic acid that encodes an engineered ACL having one or more alterations at a position described in TABLE 8. In some embodiments, the recombinant nucleic acid is an isolated nucleic acid that hybridizes under highly stringent hybridization conditions to a nucleic acid that encodes an engineered ACL having one or more or a combination of alterations described in TABLE 9.

In some embodiments, provided herein is a recombinant nucleic acid encoding an engineered ACL provided herein has an amino acid sequence that is a variant of SEQ ID NO: 1 that includes one or more alterations as described in TABLE 8 and/or one or more, or a combination of alterations described in TABLE 9, wherein the portion, other than the one or more alterations described in TABLE 8 or the one or more, or a combination of alterations described in TABLE 9, of the engineered ACL has at least 65%, 70%, 75%, 80%, 85%, 90%, 95%, 98% or 99% sequence identity, or is identical, to an amino acid sequence referenced as SEQ ID NO: 1. Accordingly, in some embodiments, a recombinant nucleic acid encodes an engineered ACL that has an amino acid sequence that includes one or more alterations as described in TABLE 8 and/or one or more, or a combination of alterations, described in TABLE 9 and the portion, other than the alteration described in TABLE 8 or the one or more, or combination of alterations, described in TABLE 9, of the engineered ACL has at least 65% identical to SEQ ID NO: 1. In some embodiments, a recombinant nucleic acid encodes an engineered ACL that has an amino acid sequence that includes one or more alterations as described in TABLE 8 and/or one or more, or a combination of alterations, described in TABLE 9 and the portion, other than the alteration described in TABLE 8 and/or the one or more, or combination of alterations, described in TABLE 9, of the engineered ACL has at least 70% identical to SEQ ID NO: 1. In some embodiments, a recombinant nucleic acid encodes an engineered ACL that has an amino acid sequence that includes one or more alterations as described in TABLE 8 and/or one or more, or a combination of alterations, described in TABLE 9 and the portion, other than the alteration described in TABLE 8 and/or the one or more, or combination of alterations, described in TABLE 9, of the engineered ACL has at least 75% identical to SEQ ID NO: 1. In some embodiments, a recombinant nucleic acid encodes an engineered ACL that has an amino acid sequence that includes one or more alterations as described in TABLE 8 and/or one or more, or a combination of alterations, described in TABLE 9 and the portion, other than the alteration described in TABLE 8 and/or the one or more, or combination of alterations, described in TABLE 9, of the engineered ACL has at least 80% identical to SEQ ID NO: 1. In some embodiments, a recombinant nucleic acid encodes an engineered ACL that has an amino acid sequence that includes one or more alterations as described in TABLE 8 and/or one or more, or a combination of alterations, described in TABLE 9 and the portion, other than the alteration described in TABLE 8 and/or the one or more, or combination of alterations, described in TABLE 9, of the engineered ACL has at least 85% identical to SEQ ID NO: 1. In some embodiments, a recombinant nucleic acid encodes an engineered ACL that has an amino acid sequence that includes one or more alterations as described in TABLE 8 and/or one or more, or a combination of alterations, described in TABLE 9 and the portion, other than the alteration described in TABLE 8 and/or the one or more, or combination of alterations, described in TABLE 9, of the engineered ACL has at least 90% identical to SEQ ID NO: 1. In some embodiments, a recombinant nucleic acid encodes an engineered ACL that has an amino acid sequence that includes one or more alterations as described in TABLE 8 and/or one or more, or a combination of alterations, described in TABLE 9 and the portion, other than the alteration described in TABLE 8 and/or the one or more, or combination of alterations, described in TABLE 9, of the engineered ACL has at least 95% identical to SEQ ID NO: 1. In some embodiments, a recombinant nucleic acid encodes an engineered ACL that has an amino acid sequence that includes one or more alterations as described in TABLE 8 and/or one or more, or a combination of alterations, described in TABLE 9 and the portion, other than the alteration described in TABLE 8 and/or the one or more, or combination of alterations, described in TABLE 9, of the engineered ACL has at least 98% identical to SEQ ID NO: 1. In some embodiments, a recombinant nucleic acid encodes an engineered ACL that has an amino acid sequence that includes one or more alterations as described in TABLE 8 and/or one or more, or a combination of alterations, described in TABLE 9 and the portion, other than the alteration described in TABLE 8 and/or the one or more, or combination of alterations, described in TABLE 9, of the engineered ACL has at least 99% identical to SEQ ID NO: 1. In some embodiments, a recombinant nucleic acid encodes an engineered ACL that has an amino acid sequence that includes one or more alterations as described in TABLE 8 and/or one or more, or a combination of alterations, described in TABLE 9 and the portion, other than the alteration described in TABLE 8 and/or the one or more, or combination of alterations, described in TABLE 9, of the engineered ACL is identical to SEQ ID NO: 1.

In some embodiments, provided herein is a recombinant nucleic acid encoding an engineered ACL provided herein has an amino acid sequence that is a variant of SEQ ID NO: 2 that includes one or more alterations as described in TABLE 9 and/or one or more, or a combination of alterations described in TABLE 8, wherein the portion, other than the one or more alterations described in TABLE 9 or the one or more, or a combination of alterations described in TABLE 8, of the engineered ACL has at least 65%, 70%, 75%, 80%, 85%, 90%, 95%, 98% or 99% sequence identity, or is identical, to an amino acid sequence referenced as SEQ ID NO: 2. Accordingly, in some embodiments, a recombinant nucleic acid encodes an engineered ACL that has an amino acid sequence that includes one or more alterations as described in TABLE 9 and/or one or more, or a combination of alterations, described in TABLE 8 and the portion, other than the alteration described in TABLE 9 or the one or more, or combination of alterations, described in TABLE 8, of the engineered ACL has at least 65% identical to SEQ ID NO: 2. In some embodiments, a recombinant nucleic acid encodes an engineered ACL that has an amino acid sequence that includes one or more alterations as described in TABLE 9 and/or one or more, or a combination of alterations, described in TABLE 8 and the portion, other than the alteration described in TABLE 9 and/or the one or more, or combination of alterations, described in TABLE 8, of the engineered ACL has at least 70% identical to SEQ ID NO: 2. In some embodiments, a recombinant nucleic acid encodes an engineered ACL that has an amino acid sequence that includes one or more alterations as described in TABLE 9 and/or one or more, or a combination of alterations, described in TABLE 8 and the portion, other than the alteration described in TABLE 9 and/or the one or more, or combination of alterations, described in TABLE 8, of the engineered ACL has at least 75% identical to SEQ ID NO: 2. In some embodiments, a recombinant nucleic acid encodes an engineered ACL that has an amino acid sequence that includes one or more alterations as described in TABLE 9 and/or one or more, or a combination of alterations, described in TABLE 8 and the portion, other than the alteration described in TABLE 9 and/or the one or more, or combination of alterations, described in TABLE 8, of the engineered ACL has at least 80% identical to SEQ ID NO: 2. In some embodiments, a recombinant nucleic acid encodes an engineered ACL that has an amino acid sequence that includes one or more alterations as described in TABLE 9 and/or one or more, or a combination of alterations, described in TABLE 8 and the portion, other than the alteration described in TABLE 9 and/or the one or more, or combination of alterations, described in TABLE 8, of the engineered ACL has at least 85% identical to SEQ ID NO: 2. In some embodiments, a recombinant nucleic acid encodes an engineered ACL that has an amino acid sequence that includes one or more alterations as described in TABLE 9 and/or one or more, or a combination of alterations, described in TABLE 8 and the portion, other than the alteration described in TABLE 9 and/or the one or more, or combination of alterations, described in TABLE 8, of the engineered ACL has at least 90% identical to SEQ ID NO: 2. In some embodiments, a recombinant nucleic acid encodes an engineered ACL that has an amino acid sequence that includes one or more alterations as described in TABLE 9 and/or one or more, or a combination of alterations, described in TABLE 8 and the portion, other than the alteration described in TABLE 9 and/or the one or more, or combination of alterations, described in TABLE 8, of the engineered ACL has at least 95% identical to SEQ ID NO: 2. In some embodiments, a recombinant nucleic acid encodes an engineered ACL that has an amino acid sequence that includes one or more alterations as described in TABLE 9 and/or one or more, or a combination of alterations, described in TABLE 8 and the portion, other than the alteration described in TABLE 9 and/or the one or more, or combination of alterations, described in TABLE 8, of the engineered ACL has at least 98% identical to SEQ ID NO: 2. In some embodiments, a recombinant nucleic acid encodes an engineered ACL that has an amino acid sequence that includes one or more alterations as described in TABLE 9 and/or one or more, or a combination of alterations, described in TABLE 8 and the portion, other than the alteration described in TABLE 9 and/or the one or more, or combination of alterations, described in TABLE 8, of the engineered ACL has at least 99% identical to SEQ ID NO: 2. In some embodiments, a recombinant nucleic acid encodes an engineered ACL that has an amino acid sequence that includes one or more alterations as described in TABLE 9 and/or one or more, or a combination of alterations, described in TABLE 8 and the portion, other than the alteration described in TABLE 9 and/or the one or more, or combination of alterations, described in TABLE 8, of the engineered ACL is identical to SEQ ID NO: 2.

In some embodiments, provided herein is a recombinant nucleic acid that has a nucleotide sequence encoding an engineered ALDH described herein. Accordingly, in some embodiments, provided herein is a recombinant nucleic acid selected from (a) a nucleic acid molecule encoding an engineered ACL that is a variant of a wild-type ACL (SEQ ID NO: 3), such as an engineered ACL having one or more alterations at a position described in TABLE 12; (b) a recombinant nucleic acid that hybridizes to an isolated nucleic acid of (a) under highly stringent hybridization conditions; and (c) a recombinant nucleic acid that is complementary to (a) or (b).

In some embodiments, provided herein is a recombinant nucleic acid encoding an engineered ALDH that is a variant of a reference polypeptide, wherein the reference polypeptide has an amino acid sequence of SEQ ID NO: 3, and the engineered ACL has one or more alterations at a position described in TABLE 12 relative to SEQ ID NO: 3. In some embodiments, the recombinant nucleic acid encodes an engineered ALDH that includes one or more amino acid alterations at a residue corresponding to position 2, 3, 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, 36, 37, 38, 39, 40, 41, 42, 43, 44, 45, 47, 48, 49, 50, 51, 52, 53, 54, 55, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 104, 106, 107, 108, 109, 110, 111, 112, 113, 114, 115, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, 201, 202, 203, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 243, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 288, 289, 290, 291, 292, 293, 294, 295, 296, 297, 298, 299, 300, 301, 302, 303, 304, 305, 306, 307, 308, 310, 311, 312, 313, 314, 315, 316, 317, 318, 319, 320, 321, 322, 323, 324, 325, 326, 327, 328, 329, 330, 331, 332, 333, 334, 335, 337, 338, 339, 341, 342, 343, 344, 345, 346, 347, 348, 349, 350, 351, 352, 354, 355, 356, 357, 358, 359, 360, 361, 362, 363, 364, 366, 368, 369, 370, 371, 372, 373, 374, 375, 376, 377, 378, 379, 380, 381, 382, 383, 384, 385, 386, 387, 388, 389, 390, 391, 392, 393, 394, 396, 397, 398, 399, 400, 401, 402, 403, 405, 406, 407, 408, 410, 411, 412, 413, 414, 415, 416, 418, 420, 421, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, more than one of the foregoing, or a combination thereof, in SEQ ID NO: 3. In some embodiments, the recombinant nucleic acid encodes an engineered ALDH that includes one or more amino acid alterations at a residue corresponding to position 2, 3, 4, 5, 6, 7, 9, 11, 12, 15, 16, 17, 18, 20, 21, 22, 23, 24, 25, 26, 27, 28, 30, 31, 32, 33, 34, 35, 36, 37, 38, 39, 40, 41, 42, 43, 44, 45, 47, 48, 49, 51, 52, 54, 55, 58, 61, 62, 63, 64, 65, 66, 67, 70, 71, 73, 74, 75, 77, 78, 79, 80, 81, 82, 83, 84, 86, 88, 89, 90, 91, 92, 93, 94, 95, 96, 97, 98, 99, 100, 101, 102, 104, 107, 111, 117, 119, 120, 121, 122, 124, 125, 126, 128, 129, 130, 133, 136, 144, 145, 146, 147, 148, 149, 150, 151, 152, 153, 154, 155, 156, 157, 158, 159, 160, 162, 164, 165, 166, 167, 168, 169, 170, 171, 172, 174, 175, 176, 177, 180, 181, 183, 184, 187, 189, 190, 194, 196, 197, 198, 199, 200, 201, 202, 203, 206, 207, 208, 212, 214, 215, 216, 217, 218, 219, 221, 222, 223, 224, 225, 227, 228, 229, 230, 231, 232, 233, 234, 237, 238, 240, 243, 245, 246, 248, 249, 250, 251, 252, 253, 254, 255, 256, 257, 258, 259, 260, 261, 262, 263, 264, 265, 266, 267, 270, 271, 272, 273, 274, 275, 276, 277, 278, 279, 280, 281, 282, 283, 284, 285, 286, 288, 290, 291, 293, 294, 295, 296, 299, 300, 301, 302, 303, 304, 305, 306, 307, 308, 310, 311, 312, 313, 315, 316, 317, 318, 319, 320, 321, 322, 323, 324, 325, 326, 327, 328, 329, 330, 331, 332, 333, 334, 335, 339, 341, 342, 343, 344, 345, 346, 347, 348, 349, 350, 351, 354, 355, 356, 358, 359, 360, 362, 363, 366, 370, 371, 372, 373, 374, 375, 376, 377, 378, 379, 380, 381, 382, 383, 384, 385, 386, 387, 388, 389, 390, 392, 393, 394, 396, 397, 400, 401, 402, 405, 406, 407, 410, 411, 414, 420, 421, 423, 425, 426, 427, 428, 431, 433, 435, 436, 437, 438, 439, 440, 441, 443, 444, 446, 447, 448, 449, 450, 452, 453, 454, 455, 457, 458, 459, more than one of the foregoing, or a combination thereof, in SEQ ID NO: 3. In some embodiments, the recombinant nucleic acid encodes an engineered ALDH that includes one or more amino acid alterations at a residue corresponding to position 42, 119, 149, 158, 174, 246, 259, 263, 299, 327, 348, 370, 392, 401, 427, more than one of the foregoing, or a combination thereof, in SEQ ID NO: 3.

In some embodiments, the recombinant nucleic acid encodes an engineered ALDH having one or more alterations described in TABLE 12. Accordingly, in some embodiments, the recombinant nucleic acid encodes an engineered ALDH having: P, Q, T at a residue corresponding to position 2 in SEQ ID NO: 3; E, K, M, R, S at a residue corresponding to position 3 in SEQ ID NO: 3; G, S at a residue corresponding to position 4 in SEQ ID NO: 3; I, T at a residue corresponding to position 5 in SEQ ID NO: 3; E, F, H, P, Q, R at a residue corresponding to position 6 in SEQ ID NO: 3; E, Q at a residue corresponding to position 7 in SEQ ID NO: 3; D at a residue corresponding to position 8 in SEQ ID NO: 3; I, V at a residue corresponding to position 9 in SEQ ID NO: 3; N at a residue corresponding to position 10 in SEQ ID NO: 3; A, D, E, Q, R at a residue corresponding to position 11 in SEQ ID NO: 3; A, I, L at a residue corresponding to position 12 in SEQ ID NO: 3; L at a residue corresponding to position 13 in SEQ ID NO: 3; D, E, K, Q at a residue corresponding to position 14 in SEQ ID NO: 3; K, R at a residue corresponding to position 15 in SEQ ID NO: 3; A at a residue corresponding to position 16 in SEQ ID NO: 3; H, K, Q at a residue corresponding to position 17 in SEQ ID NO: 3; E, K, Q, T at a residue corresponding to position 18 in SEQ ID NO: 3; F, H, M at a residue corresponding to position 20 in SEQ ID NO: 3; A, E, K, P, R at a residue corresponding to position 21 in SEQ ID NO: 3; A, K, Q at a residue corresponding to position 22 in SEQ ID NO: 3; L, W, Y at a residue corresponding to position 23 in SEQ ID NO: 3; E, K, R, S at a residue corresponding to position 24 in SEQ ID NO: 3; A, E, K, N, S at a residue corresponding to position 25 in SEQ ID NO: 3; F, L, M, T, W at a residue corresponding to position 26 in SEQ ID NO: 3; D, S, T at a residue corresponding to position 27 in SEQ ID NO: 3; W at a residue corresponding to position 28 in SEQ ID NO: 3; K, Q at a residue corresponding to position 29 in SEQ ID NO: 3; D, E, K, Q, R at a residue corresponding to position 30 in SEQ ID NO: 3; I, L, R at a residue corresponding to position 31 in SEQ ID NO: 3; A, E, N at a residue corresponding to position 32 in SEQ ID NO: 3; A, E, Q, R at a residue corresponding to position 33 in SEQ ID NO: 3; I, L, M, V at a residue corresponding to position 34 in SEQ ID NO: 3; C, I, L, M at a residue corresponding to position 35 in SEQ ID NO: 3; E, H, K at a residue corresponding to position 36 in SEQ ID NO: 3; A, K, R at a residue corresponding to position 37 in SEQ ID NO: 3; F, I, L, M, W at a residue corresponding to position 38 in SEQ ID NO: 3; A, R at a residue corresponding to position 39 in SEQ ID NO: 3; D, E, Q, R, W at a residue corresponding to position 40 in SEQ ID NO: 3; L, V at a residue corresponding to position 41 in SEQ ID NO: 3; C, L, M, V at a residue corresponding to position 42 in SEQ ID NO: 3; E, L, R at a residue corresponding to position 43 in SEQ ID NO: 3; E, K, Q, R at a residue corresponding to position 44 in SEQ ID NO: 3; H, Q, R at a residue corresponding to position 45 in SEQ ID NO: 3; D at a residue corresponding to position 47 in SEQ ID NO: 3; D, E, M, R at a residue corresponding to position 48 in SEQ ID NO: 3; F, I, M, W, Y at a residue corresponding to position 49 in SEQ ID NO: 3; G at a residue corresponding to position 50 in SEQ ID NO: 3; A, E, K, N, Q, R at a residue corresponding to position 51 in SEQ ID NO: 3; L at a residue corresponding to position 52 in SEQ ID NO: 3; E, I, S at a residue corresponding to position 53 in SEQ ID NO: 3; I, L, M, T at a residue corresponding to position 54 in SEQ ID NO: 3; E, Q, R at a residue corresponding to position 55 in SEQ ID NO: 3; E, G, Q, R at a residue corresponding to position 58 in SEQ ID NO: 3; F, I, L, Y at a residue corresponding to position 59 in SEQ ID NO: 3; A at a residue corresponding to position 60 in SEQ ID NO: 3; D, E, K, R at a residue corresponding to position 61 in SEQ ID NO: 3; A, F, I, M, V, W at a residue corresponding to position 62 in SEQ ID NO: 3; D, E, Q at a residue corresponding to position 63 in SEQ ID NO: 3; E, H at a residue corresponding to position 64 in SEQ ID NO: 3; E, R at a residue corresponding to position 65 in SEQ ID NO: 3; L, V at a residue corresponding to position 66 in SEQ ID NO: 3; E at a residue corresponding to position 67 in SEQ ID NO: 3; D, E at a residue corresponding to position 68 in SEQ ID NO: 3; H, I, L, M, N at a residue corresponding to position 69 in SEQ ID NO: 3; I, L, M, R at a residue corresponding to position 70 in SEQ ID NO: 3; D, E, F, H, Y at a residue corresponding to position 71 in SEQ ID NO: 3; A, R at a residue corresponding to position 72 in SEQ ID NO: 3; A, T at a residue corresponding to position 73 in SEQ ID NO: 3; D, E, R at a residue corresponding to position 74 in SEQ ID NO: 3; G, P, Y at a residue corresponding to position 75 in SEQ ID NO: 3; F, I, L, M, Y at a residue corresponding to position 76 in SEQ ID NO: 3; F, Y at a residue corresponding to position 77 in SEQ ID NO: 3; E, F, H, N, R, Y at a residue corresponding to position 78 in SEQ ID NO: 3; D, S, W, Y at a residue corresponding to position 79 in SEQ ID NO: 3; F, I, L, V at a residue corresponding to position 80 in SEQ ID NO: 3; A, E, R at a residue corresponding to position 81 in SEQ ID NO: 3; E, G, P at a residue corresponding to position 82 in SEQ ID NO: 3; E, Q at a residue corresponding to position 83 in SEQ ID NO: 3; Q, R at a residue corresponding to position 84 in SEQ ID NO: 3; A, T at a residue corresponding to position 85 in SEQ ID NO: 3; E, K, V at a residue corresponding to position 86 in SEQ ID NO: 3; T at a residue corresponding to position 87 in SEQ ID NO: 3; L, M, V at a residue corresponding to position 88 in SEQ ID NO: 3; L, P, V at a residue corresponding to position 89 in SEQ ID NO: 3; D, E, H, P, R, S at a residue corresponding to position 90 in SEQ ID NO: 3; D, E, Q at a residue corresponding to position 91 in SEQ ID NO: 3; D, M at a residue corresponding to position 92 in SEQ ID NO: 3; D, E at a residue corresponding to position 93 in SEQ ID NO: 3; A, D, G, N at a residue corresponding to position 94 in SEQ ID NO: 3; K, M, N, R at a residue corresponding to position 95 in SEQ ID NO: 3; H, K, N, R at a residue corresponding to position 96 in SEQ ID NO: 3; I, M, V at a residue corresponding to position 97 in SEQ ID NO: 3; I, M, T, Y at a residue corresponding to position 98 in SEQ ID NO: 3; K, Q, R, T at a residue corresponding to position 99 in SEQ ID NO: 3; Y, L, M, I at a residue corresponding to position 100 in SEQ ID NO: 3; K, Y, R, H at a residue corresponding to position 101 in SEQ ID NO: 3; E, Q, R, V at a residue corresponding to position 102 in SEQ ID NO: 3; V, Y, F, L, M at a residue corresponding to position 104 in SEQ ID NO: 3; I at a residue corresponding to position 106 in SEQ ID NO: 3; C, V at a residue corresponding to position 107 in SEQ ID NO: 3; A, C at a residue corresponding to position 108 in SEQ ID NO: 3; I, A, M at a residue corresponding to position 109 in SEQ ID NO: 3; M, L, I at a residue corresponding to position 110 in SEQ ID NO: 3; T, V, Y, I, S at a residue corresponding to position 111 in SEQ ID NO: 3; E at a residue corresponding to position 112 in SEQ ID NO: 3; F, S, W, Y at a residue corresponding to position 113 in SEQ ID NO: 3; N at a residue corresponding to position 114 in SEQ ID NO: 3; W, R, H, Y at a residue corresponding to position 115 in SEQ ID NO: 3; N, L, T, I at a residue corresponding to position 117 in SEQ ID NO: 3; N, A, L, M, S, F, V at a residue corresponding to position 118 in SEQ ID NO: 3; Q, I, L, M, T, S at a residue corresponding to position 119 in SEQ ID NO: 3; T, A, P, M at a residue corresponding to position 120 in SEQ ID NO: 3; V, A, L, C, I at a residue corresponding to position 121 in SEQ ID NO: 3; C, F, R, Y, H, N, S, G, V, W at a residue corresponding to position 122 in SEQ ID NO: 3; K, P at a residue corresponding to position 123 in SEQ ID NO: 3; V, L, M, I at a residue corresponding to position 124 in SEQ ID NO: 3; I, L, A, V at a residue corresponding to position 125 in SEQ ID NO: 3; A, P at a residue corresponding to position 126 in SEQ ID NO: 3; M at a residue corresponding to position 127 in SEQ ID NO: 3; I at a residue corresponding to position 128 in SEQ ID NO: 3; A, M at a residue corresponding to position 129 in SEQ ID NO: 3; T, S, A at a residue corresponding to position 130 in SEQ ID NO: 3; R at a residue corresponding to position 131 in SEQ ID NO: 3; C at a residue corresponding to position 132 in SEQ ID NO: 3; T, C at a residue corresponding to position 133 in SEQ ID NO: 3; I, C, V at a residue corresponding to position 134 in SEQ ID NO: 3; V, M at a residue corresponding to position 135 in SEQ ID NO: 3; L, M, F, I at a residue corresponding to position 136 in SEQ ID NO: 3; R, K at a residue corresponding to position 137 in SEQ ID NO: 3; G at a residue corresponding to position 138 in SEQ ID NO: 3; S, G at a residue corresponding to position 139 in SEQ ID NO: 3; E at a residue corresponding to position 140 in SEQ ID NO: 3; Q, K, Y, E at a residue corresponding to position 141 in SEQ ID NO: 3; T, S at a residue corresponding to position 142 in SEQ ID NO: 3; P at a residue corresponding to position 143 in SEQ ID NO: 3; L, N, R at a residue corresponding to position 144 in SEQ ID NO: 3; T, C, V at a residue corresponding to position 145 in SEQ ID NO: 3; T, C, A, N at a residue corresponding to position 146 in SEQ ID NO: 3; I, M, A at a residue corresponding to position 147 in SEQ ID NO: 3; Y, L, A, E, H, R at a residue corresponding to position 148 in SEQ ID NO: 3; V, I, L, M, F at a residue corresponding to position 149 in SEQ ID NO: 3; I, M, G, A at a residue corresponding to position 150 in SEQ ID NO: 3; D, K, R, Q at a residue corresponding to position 151 in SEQ ID NO: 3; L, M, I at a residue corresponding to position 152 in SEQ ID NO: 3; F, V, C, M at a residue corresponding to position 153 in SEQ ID NO: 3; H, A, Q, K, R at a residue corresponding to position 154 in SEQ ID NO: 3; R, K, D, Q at a residue corresponding to position 155 in SEQ ID NO: 3; I at a residue corresponding to position 156 in SEQ ID NO: 3; A, I, F at a residue corresponding to position 157 in SEQ ID NO: 3; E, V, K at a residue corresponding to position 158 in SEQ ID NO: 3; K, Q, D, E at a residue corresponding to position 159 in SEQ ID NO: 3; V, C, A at a residue corresponding to position 160 in SEQ ID NO: 3; I, A, F, Y, L, V at a residue corresponding to position 162 in SEQ ID NO: 3; D, A, P at a residue corresponding to position 164 in SEQ ID NO: 3; H, G, D at a residue corresponding to position 165 in SEQ ID NO: 3; C, A, I, V at a residue corresponding to position 166 in SEQ ID NO: 3; V, F, L at a residue corresponding to position 167 in SEQ ID NO: 3; T, N at a residue corresponding to position 168 in SEQ ID NO: 3; L, W, C, M, I at a residue corresponding to position 169 in SEQ ID NO: 3; L, V at a residue corresponding to position 170 in SEQ ID NO: 3; E, P, N, D at a residue corresponding to position 171 in SEQ ID NO: 3; D, G, H at a residue corresponding to position 172 in SEQ ID NO: 3; G, D at a residue corresponding to position 173 in SEQ ID NO: 3; G, D, T at a residue corresponding to position 174 in SEQ ID NO: 3; I, H, R at a residue corresponding to position 175 in SEQ ID NO: 3; Q, D at a residue corresponding to position 176 in SEQ ID NO: 3; V, T, A, I at a residue corresponding to position 177 in SEQ ID NO: 3; V, A, T, G at a residue corresponding to position 178 in SEQ ID NO: 3; D, N, E at a residue corresponding to position 179 in SEQ ID NO: 3; H, Q, A, E at a residue corresponding to position 180 in SEQ ID NO: 3; I, F at a residue corresponding to position 181 in SEQ ID NO: 3; L, H, I, V at a residue corresponding to position 182 in SEQ ID NO: 3; Q, K, D, A, H, E at a residue corresponding to position 183 in SEQ ID NO: 3; H, P, S, G at a residue corresponding to position 184 in SEQ ID NO: 3; C, I at a residue corresponding to position 185 in SEQ ID NO: 3; R at a residue corresponding to position 186 in SEQ ID NO: 3; H, K, F, I, V, M at a residue corresponding to position 187 in SEQ ID NO: 3; I, L, V at a residue corresponding to position 188 in SEQ ID NO: 3; S, F, T, M, V, L at a residue corresponding to position 189 in SEQ ID NO: 3; P, A, F at a residue corresponding to position 190 in SEQ ID NO: 3; R at a residue corresponding to position 191 in SEQ ID NO: 3; S at a residue corresponding to position 193 in SEQ ID NO: 3; T, R, K, A at a residue corresponding to position 194 in SEQ ID NO: 3; A at a residue corresponding to position 195 in SEQ ID NO: 3; M, I, V at a residue corresponding to position 196 in SEQ ID NO: 3; I, M, G, A at a residue corresponding to position 197 in SEQ ID NO: 3; Q, E, R at a residue corresponding to position 198 in SEQ ID NO: 3; S, V at a residue corresponding to position 199 in SEQ ID NO: 3; C, I, V at a residue corresponding to position 200 in SEQ ID NO: 3; M, Q, A at a residue corresponding to position 201 in SEQ ID NO: 3; N, K, A, R, T, E, Q at a residue corresponding to position 202 in SEQ ID NO: 3; N, H, A at a residue corresponding to position 203 in SEQ ID NO: 3; R, T at a residue corresponding to position 205 in SEQ ID NO: 3; V, R, K at a residue corresponding to position 206 in SEQ ID NO: 3; V, S, C, T at a residue corresponding to position 207 in SEQ ID NO: 3; V, T, Y, M, L at a residue corresponding to position 208 in SEQ ID NO: 3; L, E, M at a residue corresponding to position 209 in SEQ ID NO: 3; H, L at a residue corresponding to position 210 in SEQ ID NO: 3; L at a residue corresponding to position 211 in SEQ ID NO: 3; A, P, G at a residue corresponding to position 212 in SEQ ID NO: 3; K at a residue corresponding to position 213 in SEQ ID NO: 3; S, K at a residue corresponding to position 214 in SEQ ID NO: 3; S, N, A, C, D, P at a residue corresponding to position 215 in SEQ ID NO: 3; H, A, P at a residue corresponding to position 216 in SEQ ID NO: 3; F, A, I, C, M at a residue corresponding to position 217 in SEQ ID NO: 3; I, V, F, Y at a residue corresponding to position 218 in SEQ ID NO: 3; M, F, L, I at a residue corresponding to position 219 in SEQ ID NO: 3; C, F at a residue corresponding to position 220 in SEQ ID NO: 3; D, P at a residue corresponding to position 221 in SEQ ID NO: 3; T, S at a residue corresponding to position 222 in SEQ ID NO: 3; C at a residue corresponding to position 223 in SEQ ID NO: 3; N at a residue corresponding to position 224 in SEQ ID NO: 3; V at a residue corresponding to position 225 in SEQ ID NO: 3; E at a residue corresponding to position 226 in SEQ ID NO: 3; K, R, M, E at a residue corresponding to position 227 in SEQ ID NO: 3; A at a residue corresponding to position 228 in SEQ ID NO: 3; W, V at a residue corresponding to position 229 in SEQ ID NO: 3; R, D, P, Q, K, E at a residue corresponding to position 230 in SEQ ID NO: 3; Q, R, I, K at a residue corresponding to position 231 in SEQ ID NO: 3; I, F, T, C, Q, M, N at a residue corresponding to position 232 in SEQ ID NO: 3; V, L, M at a residue corresponding to position 233 in SEQ ID NO: 3; W, Q, F, A, M at a residue corresponding to position 234 in SEQ ID NO: 3; A, S at a residue corresponding to position 235 in SEQ ID NO: 3; K, C, W, A, I at a residue corresponding to position 236 in SEQ ID NO: 3; F, Y, C, M at a residue corresponding to position 237 in SEQ ID NO: 3; R, M, Q, Y, W at a residue corresponding to position 238 in SEQ ID NO: 3; R, H, Q at a residue corresponding to position 239 in SEQ ID NO: 3; Q, A, T, C, S at a residue corresponding to position 240 in SEQ ID NO: 3; N at a residue corresponding to position 241 in SEQ ID NO: 3; V, M, T, C at a residue corresponding to position 243 in SEQ ID NO: 3; M, I, N, S, V, A, C at a residue corresponding to position 245 in SEQ ID NO: 3; S, A at a residue corresponding to position 246 in SEQ ID NO: 3; G, P, I, A at a residue corresponding to position 247 in SEQ ID NO: 3; D, T, E, K, S, N at a residue corresponding to position 248 in SEQ ID NO: 3; Y, R at a residue corresponding to position 249 in SEQ ID NO: 3; I, L, M at a residue corresponding to position 250 in SEQ ID NO: 3; Y, F, L, V at a residue corresponding to position 251 in SEQ ID NO: 3; V, I at a residue corresponding to position 252 in SEQ ID NO: 3; D, Q, H, V, E at a residue corresponding to position 253 in SEQ ID NO: 3; Q, K, R, D at a residue corresponding to position 254 in SEQ ID NO: 3; E at a residue corresponding to position 255 in SEQ ID NO: 3; V, M, I, R at a residue corresponding to position 256 in SEQ ID NO: 3; A, F, H, Y at a residue corresponding to position 257 in SEQ ID NO: 3; D at a residue corresponding to position 258 in SEQ ID NO: 3; A, R, K, D, Q at a residue corresponding to position 259 in SEQ ID NO: 3; I, V, F, L at a residue corresponding to position 260 in SEQ ID NO: 3; L, M, V at a residue corresponding to position 261 in SEQ ID NO: 3; Q, D, E, K at a residue corresponding to position 262 in SEQ ID NO: 3; E, K, R at a residue corresponding to position 263 in SEQ ID NO: 3; M, V, F at a residue corresponding to position 264 in SEQ ID NO: 3; Q, R, A, E at a residue corresponding to position 265 in SEQ ID NO: 3; A, E at a residue corresponding to position 266 in SEQ ID NO: 3; Y, R, N, A, Q at a residue corresponding to position 267 in SEQ ID NO: 3; A at a residue corresponding to position 268 in SEQ ID NO: 3; S, G, C at a residue corresponding to position 269 in SEQ ID NO: 3; L, F, I, M, W at a residue corresponding to position 270 in SEQ ID NO: 3; F, K, V, W, R, H at a residue corresponding to position 271 in SEQ ID NO: 3; V, M, L at a residue corresponding to position 272 in SEQ ID NO: 3; C at a residue corresponding to position 273 in SEQ ID NO: 3; E, D at a residue corresponding to position 274 in SEQ ID NO: 3; P, G, A, E at a residue corresponding to position 275 in SEQ ID NO: 3; Q, M, R, D at a residue corresponding to position 276 in SEQ ID NO: 3; Q, E at a residue corresponding to position 277 in SEQ ID NO: 3; P, E at a residue corresponding to position 278 in SEQ ID NO: 3; E, D, S at a residue corresponding to position 279 in SEQ ID NO: 3; Q, R, T at a residue corresponding to position 280 in SEQ ID NO: 3; V, M, I at a residue corresponding to position 281 in SEQ ID NO: 3; Q, K, M, G, E at a residue corresponding to position 282 in SEQ ID NO: 3; E, H, N at a residue corresponding to position 283 in SEQ ID NO: 3; V, P at a residue corresponding to position 284 in SEQ ID NO: 3; L, V, I, M at a residue corresponding to position 285 in SEQ ID NO: 3; L, I, W, Y, V at a residue corresponding to position 286 in SEQ ID NO: 3; R, D, E at a residue corresponding to position 288 in SEQ ID NO: 3; M at a residue corresponding to position 289 in SEQ ID NO: 3; H at a residue corresponding to position 290 in SEQ ID NO: 3; K, L, E, T, H, A, N at a residue corresponding to position 291 in SEQ ID NO: 3; I, M at a residue corresponding to position 292 in SEQ ID NO: 3; A, V, I, N at a residue corresponding to position 293 in SEQ ID NO: 3; S, N, P, E, R at a residue corresponding to position 294 in SEQ ID NO: 3; R, K, A, Q, D at a residue corresponding to position 295 in SEQ ID NO: 3; W, R, V, F, H, L at a residue corresponding to position 296 in SEQ ID NO: 3; A, T, Q, V, L, H, I at a residue corresponding to position 297 in SEQ ID NO: 3; A at a residue corresponding to position 298 in SEQ ID NO: 3; K, Q, R at a residue corresponding to position 299 in SEQ ID NO: 3; A, R at a residue corresponding to position 300 in SEQ ID NO: 3; I, A at a residue corresponding to position 301 in SEQ ID NO: 3; A, R, D, Q, E at a residue corresponding to position 302 in SEQ ID NO: 3; Q, W, R, E at a residue corresponding to position 303 in SEQ ID NO: 3; L, G, Y at a residue corresponding to position 304 in SEQ ID NO: 3; I, V at a residue corresponding to position 305 in SEQ ID NO: 3; H, K, E, R at a residue corresponding to position 306 in SEQ ID NO: 3; I, V, M at a residue corresponding to position 307 in SEQ ID NO: 3; A, T at a residue corresponding to position 308 in SEQ ID NO: 3; G, V, L, F at a residue corresponding to position 310 in SEQ ID NO: 3; R, T, K, S, D, E at a residue corresponding to position 311 in SEQ ID NO: 3; A, D, T, K at a residue corresponding to position 312 in SEQ ID NO: 3; L, V at a residue corresponding to position 313 in SEQ ID NO: 3; A at a residue corresponding to position 314 in SEQ ID NO: 3; D at a residue corresponding to position 315 in SEQ ID NO: 3; E, G, N at a residue corresponding to position 316 in SEQ ID NO: 3; G, T at a residue corresponding to position 317 in SEQ ID NO: 3; R, T, Y at a residue corresponding to position 318 in SEQ ID NO: 3; F, L, M, I, Y at a residue corresponding to position 319 in SEQ ID NO: 3; Y, L at a residue corresponding to position 320 in SEQ ID NO: 3; L, P, C, I at a residue corresponding to position 321 in SEQ ID NO: 3; A, T, G, V at a residue corresponding to position 322 in SEQ ID NO: 3; E, L, I, G, P, V, K at a residue corresponding to position 323 in SEQ ID NO: 3; I, V, L at a residue corresponding to position 324 in SEQ ID NO: 3; K, E, D, T at a residue corresponding to position 325 in SEQ ID NO: 3; D at a residue corresponding to position 326 in SEQ ID NO: 3; W, F, V at a residue corresponding to position 327 in SEQ ID NO: 3; D, T, S at a residue corresponding to position 328 in SEQ ID NO: 3; E, R, P, D, N at a residue corresponding to position 329 in SEQ ID NO: 3; E at a residue corresponding to position 330 in SEQ ID NO: 3; S, D, H, M, Y at a residue corresponding to position 331 in SEQ ID NO: 3; R, T at a residue corresponding to position 332 in SEQ ID NO: 3; M, V, F, I at a residue corresponding to position 333 in SEQ ID NO: 3; V, S, M, C at a residue corresponding to position 334 in SEQ ID NO: 3; R, Q, H at a residue corresponding to position 335 in SEQ ID NO: 3; Q, E at a residue corresponding to position 337 in SEQ ID NO: 3; F, V, I, M at a residue corresponding to position 338 in SEQ ID NO: 3; C, M at a residue corresponding to position 339 in SEQ ID NO: 3; I at a residue corresponding to position 341 in SEQ ID NO: 3; M, V, I at a residue corresponding to position 342 in SEQ ID NO: 3; C, T, V, S, P at a residue corresponding to position 343 in SEQ ID NO: 3; I, V, L, M at a residue corresponding to position 344 in SEQ ID NO: 3; Y, V, M, L, I at a residue corresponding to position 345 in SEQ ID NO: 3; R, K, P at a residue corresponding to position 346 in SEQ ID NO: 3; F, V at a residue corresponding to position 347 in SEQ ID NO: 3; E, K, S at a residue corresponding to position 348 in SEQ ID NO: 3; S, G, D, N, T at a residue corresponding to position 349 in SEQ ID NO: 3; V, E, I, L, F at a residue corresponding to position 350 in SEQ ID NO: 3; D at a residue corresponding to position 351 in SEQ ID NO: 3; Q, H, E at a residue corresponding to position 352 in SEQ ID NO: 3; L, V, C at a residue corresponding to position 354 in SEQ ID NO: 3; R, A, D, Q, S, E at a residue corresponding to position 355 in SEQ ID NO: 3; M, L, V at a residue corresponding to position 356 in SEQ ID NO: 3; C at a residue corresponding to position 357 in SEQ ID NO: 3; N, R at a residue corresponding to position 358 in SEQ ID NO: 3; A, D, S, N, E at a residue corresponding to position 359 in SEQ ID NO: 3; S, T at a residue corresponding to position 360 in SEQ ID NO: 3; D, V at a residue corresponding to position 361 in SEQ ID NO: 3; L, T, N, S at a residue corresponding to position 362 in SEQ ID NO: 3; Y, E at a residue corresponding to position 363 in SEQ ID NO: 3; Q, Y, F, H at a residue corresponding to position 364 in SEQ ID NO: 3; Y, K, F, A, L at a residue corresponding to position 366 in SEQ ID NO: 3; L, A at a residue corresponding to position 368 in SEQ ID NO: 3; T, A at a residue corresponding to position 369 in SEQ ID NO: 3; C, A at a residue corresponding to position 370 in SEQ ID NO: 3; M, I, A, W, S, Y at a residue corresponding to position 371 in SEQ ID NO: 3; V, M at a residue corresponding to position 372 in SEQ ID NO: 3; W, Y, F at a residue corresponding to position 373 in SEQ ID NO: 3; T at a residue corresponding to position 374 in SEQ ID NO: 3; H, Q, K, T, R at a residue corresponding to position 375 in SEQ ID NO: 3; N, S, D at a residue corresponding to position 376 in SEQ ID NO: 3; D, I, V, E at a residue corresponding to position 377 in SEQ ID NO: 3; K, D, A at a residue corresponding to position 378 in SEQ ID NO: 3; N, K, V, R at a residue corresponding to position 379 in SEQ ID NO: 3; A, M, V at a residue corresponding to position 380 in SEQ ID NO: 3; M, W, K, R, T, H at a residue corresponding to position 381 in SEQ ID NO: 3; K, R, E, Q at a residue corresponding to position 382 in SEQ ID NO: 3; M, L, Y, F, I at a residue corresponding to position 383 in SEQ ID NO: 3; S, G at a residue corresponding to position 384 in SEQ ID NO: 3; Q, L, R, K at a residue corresponding to position 385 in SEQ ID NO: 3; E, Q, K, R at a residue corresponding to position 386 in SEQ ID NO: 3; M, V, L at a residue corresponding to position 387 in SEQ ID NO: 3; R, H, D, Q, E at a residue corresponding to position 388 in SEQ ID NO: 3; A, C, T at a residue corresponding to position 389 in SEQ ID NO: 3; T, N, C, G at a residue corresponding to position 390 in SEQ ID NO: 3; M, T, I at a residue corresponding to position 391 in SEQ ID NO: 3; V, I, M at a residue corresponding to position 392 in SEQ ID NO: 3; M, I, W, L, G at a residue corresponding to position 393 in SEQ ID NO: 3; I, K at a residue corresponding to position 394 in SEQ ID NO: 3; R, E, S, D at a residue corresponding to position 396 in SEQ ID NO: 3; I, W, A, P, F, Y at a residue corresponding to position 397 in SEQ ID NO: 3; S, C, P, T at a residue corresponding to position 398 in SEQ ID NO: 3; A, S at a residue corresponding to position 399 in SEQ ID NO: 3; I, S, F, V, H at a residue corresponding to position 400 in SEQ ID NO: 3; G, H, D, S, A at a residue corresponding to position 401 in SEQ ID NO: 3; N, A, F, M at a residue corresponding to position 402 in SEQ ID NO: 3; S at a residue corresponding to position 403 in SEQ ID NO: 3; I, F, V, G at a residue corresponding to position 405 in SEQ ID NO: 3; Y, K, F, L, W at a residue corresponding to position 406 in SEQ ID NO: 3; H, Q, Y, E at a residue corresponding to position 407 in SEQ ID NO: 3; T, S, H at a residue corresponding to position 408 in SEQ ID NO: 3; F, I, M at a residue corresponding to position 410 in SEQ ID NO: 3; T, G, P, A at a residue corresponding to position 411 in SEQ ID NO: 3; F, R at a residue corresponding to position 412 in SEQ ID NO: 3; A at a residue corresponding to position 413 in SEQ ID NO: 3; M, F, V at a residue corresponding to position 414 in SEQ ID NO: 3; P, S at a residue corresponding to position 415 in SEQ ID NO: 3; M, F, I at a residue corresponding to position 416 in SEQ ID NO: 3; F, I, T at a residue corresponding to position 418 in SEQ ID NO: 3; S, R at a residue corresponding to position 420 in SEQ ID NO: 3; Y, F at a residue corresponding to position 421 in SEQ ID NO: 3; K, R, G at a residue corresponding to position 423 in SEQ ID NO: 3; S, R at a residue corresponding to position 424 in SEQ ID NO: 3; I, G, W, A at a residue corresponding to position 425 in SEQ ID NO: 3; V, T, L at a residue corresponding to position 426 in SEQ ID NO: 3; G, T, A at a residue corresponding to position 427 in SEQ ID NO: 3; D, H at a residue corresponding to position 428 in SEQ ID NO: 3; E at a residue corresponding to position 429 in SEQ ID NO: 3; I, Y, L, V at a residue corresponding to position 430 in SEQ ID NO: 3; G, S, N at a residue corresponding to position 431 in SEQ ID NO: 3; A, P, F, W at a residue corresponding to position 432 in SEQ ID NO: 3; T, R, K, H, M, E at a residue corresponding to position 433 in SEQ ID NO: 3; R, Q, D, N at a residue corresponding to position 434 in SEQ ID NO: 3; V, F, W, Y, M at a residue corresponding to position 435 in SEQ ID NO: 3; T, L, F, I, V at a residue corresponding to position 436 in SEQ ID NO: 3; H, Q, Y at a residue corresponding to position 437 in SEQ ID NO: 3; V, T, W, C, Y, I at a residue corresponding to position 438 in SEQ ID NO: 3; Q, S, R, K at a residue corresponding to position 439 in SEQ ID NO: 3; K, W at a residue corresponding to position 440 in SEQ ID NO: 3; L, I, M at a residue corresponding to position 441 in SEQ ID NO: 3; G, A at a residue corresponding to position 442 in SEQ ID NO: 3; H, W, R, F at a residue corresponding to position 443 in SEQ ID NO: 3; E, P, K at a residue corresponding to position 444 in SEQ ID NO: 3; I, Y, F, M, L at a residue corresponding to position 445 in SEQ ID NO: 3; E, K at a residue corresponding to position 446 in SEQ ID NO: 3; E, N at a residue corresponding to position 447 in SEQ ID NO: 3; W, K, P at a residue corresponding to position 448 in SEQ ID NO: 3; E, R, F, H at a residue corresponding to position 449 in SEQ ID NO: 3; M, C, F, V, L at a residue corresponding to position 450 in SEQ ID NO: 3; W at a residue corresponding to position 451 in SEQ ID NO: 3; S, D, K at a residue corresponding to position 452 in SEQ ID NO: 3; E, P, H at a residue corresponding to position 453 in SEQ ID NO: 3; A, D at a residue corresponding to position 454 in SEQ ID NO: 3; K, Q, D at a residue corresponding to position 455 in SEQ ID NO: 3; F, L, V, W, M at a residue corresponding to position 456 in SEQ ID NO: 3; M, Y, F at a residue corresponding to position 457 in SEQ ID NO: 3; H, G at a residue corresponding to position 458 in SEQ ID NO: 3; D at a residue corresponding to position 459 in SEQ ID NO: 3; more than one of the foregoing; or any combination thereof. It is understood that when alterations are listed herein for a certain position, the alteration can be in the alternative or in the conjunctive depending on the context. For example, the K, Q, D at a residue corresponding to position 455 in SEQ ID NO: 3, means that alterations at a residue corresponding to position 471 in SEQ ID NO: 3 can comprise a K or Q or D.

In some embodiments, the recombinant nucleic acid encodes an engineered ALDH having: Q, T, P at a residue corresponding to position 2 in SEQ ID NO: 3; S, K at a residue corresponding to position 3 in SEQ ID NO: 3; G, S at a residue corresponding to position 4 in SEQ ID NO: 3; I, T at a residue corresponding to position 5 in SEQ ID NO: 3; F at a residue corresponding to position 6 in SEQ ID NO: 3; E, Q at a residue corresponding to position 7 in SEQ ID NO: 3; V at a residue corresponding to position 9 in SEQ ID NO: 3; E, D, Q, R at a residue corresponding to position 11 in SEQ ID NO: 3; L, A at a residue corresponding to position 12 in SEQ ID NO: 3; R, K at a residue corresponding to position 15 in SEQ ID NO: 3; A at a residue corresponding to position 16 in SEQ ID NO: 3; K at a residue corresponding to position 17 in SEQ ID NO: 3; K, E, Q at a residue corresponding to position 18 in SEQ ID NO: 3; F, H at a residue corresponding to position 20 in SEQ ID NO: 3; A, R at a residue corresponding to position 21 in SEQ ID NO: 3; K, Q, A at a residue corresponding to position 22 in SEQ ID NO: 3; Y at a residue corresponding to position 23 in SEQ ID NO: 3; E, K at a residue corresponding to position 24 in SEQ ID NO: 3; A, E at a residue corresponding to position 25 in SEQ ID NO: 3; L, M, W at a residue corresponding to position 26 in SEQ ID NO: 3; D, T at a residue corresponding to position 27 in SEQ ID NO: 3; W at a residue corresponding to position 28 in SEQ ID NO: 3; D, K, Q at a residue corresponding to position 30 in SEQ ID NO: 3; I, L at a residue corresponding to position 31 in SEQ ID NO: 3; N, E at a residue corresponding to position 32 in SEQ ID NO: 3; E, Q, A, R at a residue corresponding to position 33 in SEQ ID NO: 3; V at a residue corresponding to position 34 in SEQ ID NO: 3; C, M at a residue corresponding to position 35 in SEQ ID NO: 3; K at a residue corresponding to position 36 in SEQ ID NO: 3; K, A, R at a residue corresponding to position 37 in SEQ ID NO: 3; L, I at a residue corresponding to position 38 in SEQ ID NO: 3; A at a residue corresponding to position 39 in SEQ ID NO: 3; W at a residue corresponding to position 40 in SEQ ID NO: 3; L, V at a residue corresponding to position 41 in SEQ ID NO: 3; L, V, M, C at a residue corresponding to position 42 in SEQ ID NO: 3; E, L at a residue corresponding to position 43 in SEQ ID NO: 3; K, Q at a residue corresponding to position 44 in SEQ ID NO: 3; H, R at a residue corresponding to position 45 in SEQ ID NO: 3; D at a residue corresponding to position 47 in SEQ ID NO: 3; E, M, D at a residue corresponding to position 48 in SEQ ID NO: 3; I at a residue corresponding to position 49 in SEQ ID NO: 3; N, E, Q at a residue corresponding to position 51 in SEQ ID NO: 3; L at a residue corresponding to position 52 in SEQ ID NO: 3; L at a residue corresponding to position 54 in SEQ ID NO: 3; E, R at a residue corresponding to position 55 in SEQ ID NO: 3; R at a residue corresponding to position 58 in SEQ ID NO: 3; D, E, R at a residue corresponding to position 61 in SEQ ID NO: 3; F, V, W at a residue corresponding to position 62 in SEQ ID NO: 3; D, E, Q at a residue corresponding to position 63 in SEQ ID NO: 3; H at a residue corresponding to position 64 in SEQ ID NO: 3; R at a residue corresponding to position 65 in SEQ ID NO: 3; V at a residue corresponding to position 66 in SEQ ID NO: 3; E at a residue corresponding to position 67 in SEQ ID NO: 3; M, R at a residue corresponding to position 70 in SEQ ID NO: 3; D, E, Y, F, H at a residue corresponding to position 71 in SEQ ID NO: 3; T at a residue corresponding to position 73 in SEQ ID NO: 3; E, R at a residue corresponding to position 74 in SEQ ID NO: 3; G at a residue corresponding to position 75 in SEQ ID NO: 3; Y at a residue corresponding to position 77 in SEQ ID NO: 3; E, F, Y, H, N at a residue corresponding to position 78 in SEQ ID NO: 3; D, W at a residue corresponding to position 79 in SEQ ID NO: 3; F, V, I at a residue corresponding to position 80 in SEQ ID NO: 3; A, R at a residue corresponding to position 81 in SEQ ID NO: 3; G at a residue corresponding to position 82 in SEQ ID NO: 3; E at a residue corresponding to position 83 in SEQ ID NO: 3; R, Q at a residue corresponding to position 84 in SEQ ID NO: 3; V, K at a residue corresponding to position 86 in SEQ ID NO: 3; M at a residue corresponding to position 88 in SEQ ID NO: 3; V, L at a residue corresponding to position 89 in SEQ ID NO: 3; S, D, H, E, R at a residue corresponding to position 90 in SEQ ID NO: 3; E, Q at a residue corresponding to position 91 in SEQ ID NO: 3; D at a residue corresponding to position 92 in SEQ ID NO: 3; D at a residue corresponding to position 93 in SEQ ID NO: 3; G, D at a residue corresponding to position 94 in SEQ ID NO: 3; K, M, R at a residue corresponding to position 95 in SEQ ID NO: 3; N at a residue corresponding to position 96 in SEQ ID NO: 3; I, V, M at a residue corresponding to position 97 in SEQ ID NO: 3; Y, I, M, T at a residue corresponding to position 98 in SEQ ID NO: 3; R, T, Q, K at a residue corresponding to position 99 in SEQ ID NO: 3; Y, I, M, L at a residue corresponding to position 100 in SEQ ID NO: 3; Y, H at a residue corresponding to position 101 in SEQ ID NO: 3; R, Q at a residue corresponding to position 102 in SEQ ID NO: 3; F, M at a residue corresponding to position 104 in SEQ ID NO: 3; C, V at a residue corresponding to position 107 in SEQ ID NO: 3; T, S at a residue corresponding to position 111 in SEQ ID NO: 3; I, T, L at a residue corresponding to position 117 in SEQ ID NO: 3; T, S at a residue corresponding to position 119 in SEQ ID NO: 3; A, T, M at a residue corresponding to position 120 in SEQ ID NO: 3; L, V, I at a residue corresponding to position 121 in SEQ ID NO: 3; H at a residue corresponding to position 122 in SEQ ID NO: 3; I, V, L, M at a residue corresponding to position 124 in SEQ ID NO: 3; I, L, V at a residue corresponding to position 125 in SEQ ID NO: 3; A at a residue corresponding to position 126 in SEQ ID NO: 3; I at a residue corresponding to position 128 in SEQ ID NO: 3; A, M at a residue corresponding to position 129 in SEQ ID NO: 3; A, S at a residue corresponding to position 130 in SEQ ID NO: 3; C, T at a residue corresponding to position 133 in SEQ ID NO: 3; L, I at a residue corresponding to position 136 in SEQ ID NO: 3; R at a residue corresponding to position 144 in SEQ ID NO: 3; C, T, V at a residue corresponding to position 145 in SEQ ID NO: 3; N at a residue corresponding to position 146 in SEQ ID NO: 3; A, I, M at a residue corresponding to position 147 in SEQ ID NO: 3; E, L, A, R at a residue corresponding to position 148 in SEQ ID NO: 3; M, V, L, I at a residue corresponding to position 149 in SEQ ID NO: 3; A, I, M at a residue corresponding to position 150 in SEQ ID NO: 3; K, D, Q at a residue corresponding to position 151 in SEQ ID NO: 3; M, I at a residue corresponding to position 152 in SEQ ID NO: 3; M, C, F, V at a residue corresponding to position 153 in SEQ ID NO: 3; Q, K at a residue corresponding to position 154 in SEQ ID NO: 3; K, R, D, Q at a residue corresponding to position 155 in SEQ ID NO: 3; I at a residue corresponding to position 156 in SEQ ID NO: 3; A, F, I at a residue corresponding to position 157 in SEQ ID NO: 3; E, V, K at a residue corresponding to position 158 in SEQ ID NO: 3; D, K at a residue corresponding to position 159 in SEQ ID NO: 3; V, A at a residue corresponding to position 160 in SEQ ID NO: 3; F, Y, V, A, L at a residue corresponding to position 162 in SEQ ID NO: 3; A, D, P at a residue corresponding to position 164 in SEQ ID NO: 3; D, H at a residue corresponding to position 165 in SEQ ID NO: 3; V, C, I at a residue corresponding to position 166 in SEQ ID NO: 3; L, V, F at a residue corresponding to position 167 in SEQ ID NO: 3; T, N at a residue corresponding to position 168 in SEQ ID NO: 3; C, L, M, I at a residue corresponding to position 169 in SEQ ID NO: 3; V at a residue corresponding to position 170 in SEQ ID NO: 3; D, E, P, N at a residue corresponding to position 171 in SEQ ID NO: 3; D, G, H at a residue corresponding to position 172 in SEQ ID NO: 3; T at a residue corresponding to position 174 in SEQ ID NO: 3; I, H at a residue corresponding to position 175 in SEQ ID NO: 3; D at a residue corresponding to position 176 in SEQ ID NO: 3; A, T, I at a residue corresponding to position 177 in SEQ ID NO: 3; E at a residue corresponding to position 180 in SEQ ID NO: 3; I at a residue corresponding to position 181 in SEQ ID NO: 3; Q, A, H, K at a residue corresponding to position 183 in SEQ ID NO: 3; G, S at a residue corresponding to position 184 in SEQ ID NO: 3; M, V, I, F, K at a residue corresponding to position 187 in SEQ ID NO: 3; V, L at a residue corresponding to position 189 in SEQ ID NO: 3; A at a residue corresponding to position 190 in SEQ ID NO: 3; A, T, R, K at a residue corresponding to position 194 in SEQ ID NO: 3; V at a residue corresponding to position 196 in SEQ ID NO: 3; I at a residue corresponding to position 197 in SEQ ID NO: 3; R at a residue corresponding to position 198 in SEQ ID NO: 3; V at a residue corresponding to position 199 in SEQ ID NO: 3; C at a residue corresponding to position 200 in SEQ ID NO: 3; A at a residue corresponding to position 201 in SEQ ID NO: 3; A, T, R at a residue corresponding to position 202 in SEQ ID NO: 3; N, H, A at a residue corresponding to position 203 in SEQ ID NO: 3; V at a residue corresponding to position 206 in SEQ ID NO: 3; V, T, C at a residue corresponding to position 207 in SEQ ID NO: 3; V, M, Y at a residue corresponding to position 208 in SEQ ID NO: 3; A, P at a residue corresponding to position 212 in SEQ ID NO: 3; S at a residue corresponding to position 214 in SEQ ID NO: 3; P, N, C, A, S at a residue corresponding to position 215 in SEQ ID NO: 3; A, P at a residue corresponding to position 216 in SEQ ID NO: 3; M, A, C at a residue corresponding to position 217 in SEQ ID NO: 3; I, V at a residue corresponding to position 218 in SEQ ID NO: 3; I, M, L at a residue corresponding to position 219 in SEQ ID NO: 3; P, D at a residue corresponding to position 221 in SEQ ID NO: 3; T, S at a residue corresponding to position 222 in SEQ ID NO: 3; C at a residue corresponding to position 223 in SEQ ID NO: 3; N at a residue corresponding to position 224 in SEQ ID NO: 3; V at a residue corresponding to position 225 in SEQ ID NO: 3; M, E, K at a residue corresponding to position 227 in SEQ ID NO: 3; A at a residue corresponding to position 228 in SEQ ID NO: 3; V at a residue corresponding to position 229 in SEQ ID NO: 3; D, E, P, K at a residue corresponding to position 230 in SEQ ID NO: 3; R, Q, I, K at a residue corresponding to position 231 in SEQ ID NO: 3; M at a residue corresponding to position 232 in SEQ ID NO: 3; L at a residue corresponding to position 233 in SEQ ID NO: 3; F, A, W, M, Q at a residue corresponding to position 234 in SEQ ID NO: 3; F, Y, C, M at a residue corresponding to position 237 in SEQ ID NO: 3; R, Q, M at a residue corresponding to position 238 in SEQ ID NO: 3; T at a residue corresponding to position 240 in SEQ ID NO: 3; V at a residue corresponding to position 243 in SEQ ID NO: 3; I at a residue corresponding to position 245 in SEQ ID NO: 3; S, A at a residue corresponding to position 246 in SEQ ID NO: 3; N at a residue corresponding to position 248 in SEQ ID NO: 3; Y at a residue corresponding to position 249 in SEQ ID NO: 3; M at a residue corresponding to position 250 in SEQ ID NO: 3; F, L at a residue corresponding to position 251 in SEQ ID NO: 3; V, I at a residue corresponding to position 252 in SEQ ID NO: 3; H at a residue corresponding to position 253 in SEQ ID NO: 3; K, R, D at a residue corresponding to position 254 in SEQ ID NO: 3; E at a residue corresponding to position 255 in SEQ ID NO: 3; I, M, V, R at a residue corresponding to position 256 in SEQ ID NO: 3; Y, A at a residue corresponding to position 257 in SEQ ID NO: 3; D at a residue corresponding to position 258 in SEQ ID NO: 3; R, A, Q, D at a residue corresponding to position 259 in SEQ ID NO: 3; V, L at a residue corresponding to position 260 in SEQ ID NO: 3; V, L, M at a residue corresponding to position 261 in SEQ ID NO: 3; K at a residue corresponding to position 262 in SEQ ID NO: 3; K, R at a residue corresponding to position 263 in SEQ ID NO: 3; V, F at a residue corresponding to position 264 in SEQ ID NO: 3; R, Q, E at a residue corresponding to position 265 in SEQ ID NO: 3; E at a residue corresponding to position 266 in SEQ ID NO: 3; R at a residue corresponding to position 267 in SEQ ID NO: 3; F, L, W, M, I at a residue corresponding to position 270 in SEQ ID NO: 3; W, F, K, H at a residue corresponding to position 271 in SEQ ID NO: 3; V, L, M at a residue corresponding to position 272 in SEQ ID NO: 3; C at a residue corresponding to position 273 in SEQ ID NO: 3; D, E at a residue corresponding to position 274 in SEQ ID NO: 3; A, P, G, E at a residue corresponding to position 275 in SEQ ID NO: 3; R, D at a residue corresponding to position 276 in SEQ ID NO: 3; E, Q at a residue corresponding to position 277 in SEQ ID NO: 3; E at a residue corresponding to position 278 in SEQ ID NO: 3; S, E at a residue corresponding to position 279 in SEQ ID NO: 3; T at a residue corresponding to position 280 in SEQ ID NO: 3; I, V, M at a residue corresponding to position 281 in SEQ ID NO: 3; K, Q, G, M at a residue corresponding to position 282 in SEQ ID NO: 3; E, N, H at a residue corresponding to position 283 in SEQ ID NO: 3; V at a residue corresponding to position 284 in SEQ ID NO: 3; V at a residue corresponding to position 285 in SEQ ID NO: 3; Y, V, I at a residue corresponding to position 286 in SEQ ID NO: 3; E at a residue corresponding to position 288 in SEQ ID NO: 3; H at a residue corresponding to position 290 in SEQ ID NO: 3; L, A, T, H, N, K at a residue corresponding to position 291 in SEQ ID NO: 3; A, I, N at a residue corresponding to position 293 in SEQ ID NO: 3; R at a residue corresponding to position 294 in SEQ ID NO: 3; D, R, Q at a residue corresponding to position 295 in SEQ ID NO: 3; L, W at a residue corresponding to position 296 in SEQ ID NO: 3; K, Q, R at a residue corresponding to position 299 in SEQ ID NO: 3; A at a residue corresponding to position 300 in SEQ ID NO: 3; A at a residue corresponding to position 301 in SEQ ID NO: 3; A, E, Q at a residue corresponding to position 302 in SEQ ID NO: 3; E, R at a residue corresponding to position 303 in SEQ ID NO: 3; L at a residue corresponding to position 304 in SEQ ID NO: 3; V at a residue corresponding to position 305 in SEQ ID NO: 3; E, H, K, R at a residue corresponding to position 306 in SEQ ID NO: 3; I, M, V at a residue corresponding to position 307 in SEQ ID NO: 3; T at a residue corresponding to position 308 in SEQ ID NO: 3; F, V, L at a residue corresponding to position 310 in SEQ ID NO: 3; D, S, R, E at a residue corresponding to position 311 in SEQ ID NO: 3; A, T, D, K at a residue corresponding to position 312 in SEQ ID NO: 3; V, L at a residue corresponding to position 313 in SEQ ID NO: 3; D at a residue corresponding to position 315 in SEQ ID NO: 3; G, E, N at a residue corresponding to position 316 in SEQ ID NO: 3; G, T at a residue corresponding to position 317 in SEQ ID NO: 3; R at a residue corresponding to position 318 in SEQ ID NO: 3; L, M at a residue corresponding to position 319 in SEQ ID NO: 3; Y, L at a residue corresponding to position 320 in SEQ ID NO: 3; I, L, C at a residue corresponding to position 321 in SEQ ID NO: 3; G, V, A, T at a residue corresponding to position 322 in SEQ ID NO: 3; I, V, K, P at a residue corresponding to position 323 in SEQ ID NO: 3; I, V, L at a residue corresponding to position 324 in SEQ ID NO: 3; E, T at a residue corresponding to position 325 in SEQ ID NO: 3; D at a residue corresponding to position 326 in SEQ ID NO: 3; F, W, V at a residue corresponding to position 327 in SEQ ID NO: 3; S at a residue corresponding to position 328 in SEQ ID NO: 3; P, R, D, N at a residue corresponding to position 329 in SEQ ID NO: 3; E at a residue corresponding to position 330 in SEQ ID NO: 3; D at a residue corresponding to position 331 in SEQ ID NO: 3; T, R at a residue corresponding to position 332 in SEQ ID NO: 3; I, M, F at a residue corresponding to position 333 in SEQ ID NO: 3; S, V, C, M at a residue corresponding to position 334 in SEQ ID NO: 3; R at a residue corresponding to position 335 in SEQ ID NO: 3; M at a residue corresponding to position 339 in SEQ ID NO: 3; I at a residue corresponding to position 341 in SEQ ID NO: 3; I, M at a residue corresponding to position 342 in SEQ ID NO: 3; S, C, V at a residue corresponding to position 343 in SEQ ID NO: 3; M, I, V, L at a residue corresponding to position 344 in SEQ ID NO: 3; L, M, Y, V at a residue corresponding to position 345 in SEQ ID NO: 3; P at a residue corresponding to position 346 in SEQ ID NO: 3; F, V at a residue corresponding to position 347 in SEQ ID NO: 3; E, K, S at a residue corresponding to position 348 in SEQ ID NO: 3; G, T, N, S, D at a residue corresponding to position 349 in SEQ ID NO: 3; E, I, V, F, L at a residue corresponding to position 350 in SEQ ID NO: 3; D at a residue corresponding to position 351 in SEQ ID NO: 3; L, C at a residue corresponding to position 354 in SEQ ID NO: 3; D, A, Q, S, E at a residue corresponding to position 355 in SEQ ID NO: 3; L, V at a residue corresponding to position 356 in SEQ ID NO: 3; R, N at a residue corresponding to position 358 in SEQ ID NO: 3; S, A, N, E at a residue corresponding to position 359 in SEQ ID NO: 3; S at a residue corresponding to position 360 in SEQ ID NO: 3; S, N, T at a residue corresponding to position 362 in SEQ ID NO: 3; E at a residue corresponding to position 363 in SEQ ID NO: 3; A, K, Y, L at a residue corresponding to position 366 in SEQ ID NO: 3; C, A at a residue corresponding to position 370 in SEQ ID NO: 3; S, A at a residue corresponding to position 371 in SEQ ID NO: 3; V, M at a residue corresponding to position 372 in SEQ ID NO: 3; F, Y, W at a residue corresponding to position 373 in SEQ ID NO: 3; T at a residue corresponding to position 374 in SEQ ID NO: 3; H, T, K, R, Q at a residue corresponding to position 375 in SEQ ID NO: 3; D at a residue corresponding to position 376 in SEQ ID NO: 3; E, I, V at a residue corresponding to position 377 in SEQ ID NO: 3; K, A, D at a residue corresponding to position 378 in SEQ ID NO: 3; R, N, V at a residue corresponding to position 379 in SEQ ID NO: 3; V at a residue corresponding to position 380 in SEQ ID NO: 3; W, H, M, R, K, T at a residue corresponding to position 381 in SEQ ID NO: 3; K at a residue corresponding to position 382 in SEQ ID NO: 3; I, M at a residue corresponding to position 383 in SEQ ID NO: 3; G, S at a residue corresponding to position 384 in SEQ ID NO: 3; R, K at a residue corresponding to position 385 in SEQ ID NO: 3; Q, K, R at a residue corresponding to position 386 in SEQ ID NO: 3; M, V, L at a residue corresponding to position 387 in SEQ ID NO: 3; R, E, Q, H, D at a residue corresponding to position 388 in SEQ ID NO: 3; T, A, C at a residue corresponding to position 389 in SEQ ID NO: 3; G, T, C at a residue corresponding to position 390 in SEQ ID NO: 3; M, V, I at a residue corresponding to position 392 in SEQ ID NO: 3; G at a residue corresponding to position 393 in SEQ ID NO: 3; I at a residue corresponding to position 394 in SEQ ID NO: 3; E, D, S at a residue corresponding to position 396 in SEQ ID NO: 3; W, I, Y at a residue corresponding to position 397 in SEQ ID NO: 3; S, I, H at a residue corresponding to position 400 in SEQ ID NO: 3; D, G, S, H, A at a residue corresponding to position 401 in SEQ ID NO: 3; M at a residue corresponding to position 402 in SEQ ID NO: 3; V, G at a residue corresponding to position 405 in SEQ ID NO: 3; W, F, L at a residue corresponding to position 406 in SEQ ID NO: 3; H, Q at a residue corresponding to position 407 in SEQ ID NO: 3; M at a residue corresponding to position 410 in SEQ ID NO: 3; G, A, P, T at a residue corresponding to position 411 in SEQ ID NO: 3; V at a residue corresponding to position 414 in SEQ ID NO: 3; S at a residue corresponding to position 420 in SEQ ID NO: 3; F at a residue corresponding to position 421 in SEQ ID NO: 3; G, R at a residue corresponding to position 423 in SEQ ID NO: 3; I, A, G at a residue corresponding to position 425 in SEQ ID NO: 3; V at a residue corresponding to position 426 in SEQ ID NO: 3; A, T at a residue corresponding to position 427 in SEQ ID NO: 3; D at a residue corresponding to position 428 in SEQ ID NO: 3; N at a residue corresponding to position 431 in SEQ ID NO: 3; T, H, K, M, R at a residue corresponding to position 433 in SEQ ID NO: 3; M, V at a residue corresponding to position 435 in SEQ ID NO: 3; V, F, I at a residue corresponding to position 436 in SEQ ID NO: 3; Q at a residue corresponding to position 437 in SEQ ID NO: 3; I, T, Y, C at a residue corresponding to position 438 in SEQ ID NO: 3; S at a residue corresponding to position 439 in SEQ ID NO: 3; W, K at a residue corresponding to position 440 in SEQ ID NO: 3; M, I, L at a residue corresponding to position 441 in SEQ ID NO: 3; W, F, R at a residue corresponding to position 443 in SEQ ID NO: 3; K at a residue corresponding to position 444 in SEQ ID NO: 3; K at a residue corresponding to position 446 in SEQ ID NO: 3; E, N at a residue corresponding to position 447 in SEQ ID NO: 3; W, K, P at a residue corresponding to position 448 in SEQ ID NO: 3; R, H at a residue corresponding to position 449 in SEQ ID NO: 3; V at a residue corresponding to position 450 in SEQ ID NO: 3; S, K at a residue corresponding to position 452 in SEQ ID NO: 3; H, P at a residue corresponding to position 453 in SEQ ID NO: 3; A, D at a residue corresponding to position 454 in SEQ ID NO: 3; K, D at a residue corresponding to position 455 in SEQ ID NO: 3; F at a residue corresponding to position 457 in SEQ ID NO: 3; H, G at a residue corresponding to position 458 in SEQ ID NO: 3; D at a residue corresponding to position 459 in SEQ ID NO: 3; more than one of the foregoing; or any combination thereof. It is understood that when alterations are listed herein for a certain position, the alteration can be in the alternative or in the conjunctive depending on the context. For example, the K, D at a residue corresponding to position 455 in SEQ ID NO: 3, means that alterations at a residue corresponding to position 471 in SEQ ID NO: 3 can comprise a K or D.

In some embodiments, the recombinant nucleic acid encodes an engineered ALDH having: V at a residue corresponding to position 42 in SEQ ID NO: 3; T or S at a residue corresponding to position 119 in SEQ ID NO: 3; I at a residue corresponding to position 149 in SEQ ID NO: 3; K at a residue corresponding to position 158 in SEQ ID NO: 3; T at a residue corresponding to position 174 in SEQ ID NO: 3; A at a residue corresponding to position 246 in SEQ ID NO: 3; D at a residue corresponding to position 259 in SEQ ID NO: 3; R at a residue corresponding to position 263 in SEQ ID NO: 3; R at a residue corresponding to position 299 in SEQ ID NO: 3; V at a residue corresponding to position 327 in SEQ ID NO: 3; S at a residue corresponding to position 348 in SEQ ID NO: 3; A at a residue corresponding to position 370 in SEQ ID NO: 3; I at a residue corresponding to position 392 in SEQ ID NO: 3; A at a residue corresponding to position 401 in SEQ ID NO: 3; T at a residue corresponding to position 427 in SEQ ID NO: 3; more than one of the foregoing; or any combination thereof.

In some embodiments, the recombinant nucleic acid encodes an engineered ALDH having one or more amino acid alterations comprising: E2P, E2Q, E2T, Q3E, Q3K, Q3M, Q3R, Q3S, A4G, A4S, V5I, V5T, K6E, K6F, K6H, K6P, K6Q, K6R, D7E, D7Q, Y8D, L9I, L9V, D10N, K11A, K11D, K11E, K11Q, K11R, M12A, M12I, M12L, V13L, A14D, A14E, A14K, A14Q, A15K, A15R, S16A, R17H, R17K, R17Q, 118E, I18K, I18Q, I18T, Q20F, Q20H, Q20M, Q21A, Q21E, Q21K, Q21P, Q21R, E22A, E22K, E22Q, F23L, F23W, F23Y, A24E, A24K, A24R, A24S, T25A, T25E, T25K, T25N, T25S, Y26F, Y26L, Y26M, Y26T, Y26W, P27D, P27S, P27T, Q28W, E29K, E29Q, T30D, T30E, T30K, T30Q, T30R, V31I, V31L, V31R, D32A, D32E, D32N, K33A, K33E, K33Q, K33R, A34I, A34L, A34M, A34V, V35C, V35I, V35L, V35M, R36E, R36H, R36K, T37A, T37K, T37R, V38F, V38I, V38L, V38M, V38W, G39A, G39R, K40D, K40E, K40Q, K40R, K40W, A41L, A41V, I42C, I42L, I42M, I42V, Y43E, Y43L, Y43R, D44E, D44K, D44Q, D44R, N45H, N45Q, N45R, E47D, L48D, L48E, L48M, L48R, L49F, L49I, L49M, L49W, L49Y, A50G, H51A, H51E, H51K, H51N, H51Q, H51R, M52L, A53E, A53I, A53S, V54I, V54L, V54M, V54T, D55E, D55Q, D55R, K58E, K58G, K58Q, K58R, M59F, M59I, M59L, M59Y, G60A, N61D, N61E, N61K, N61R, Y62A, Y62F, Y62I, Y62M, Y62V, Y62W, A63D, A63E, A63Q, D64E, D64H, K65E, K65R, I66L, I66V, A67E, K68D, K68E, C69H, C69I, C69L, C69M, C69N, V70I, V70L, V70M, V70R, N71D, N71E, N71F, N71H, N71Y, K72A, K72R, S73A, S73T, K74D, K74E, K74R, S75G, S75P, S75Y, V76F, V76I, V76L, V76M, V76Y, W77F, W77Y, W78E, W78F, W78H, W78N, W78R, W78Y, R79D, R79S, R79W, R79Y, M80F, M80I, M80L, M80V, K81A, K81E, K81R, D82E, D82G, D82P, K83E, K83Q, K84Q, K84R, S85A, S85T, R86E, R86K, R86V, G87T, I88L, I88M, I88V, I89L, I89P, I89V, K90D, K90E, K90H, K90P, K90R, K90S, R91D, R91E, R91Q, I92D, I92M, P93D, P93E, E94A, E94D, E94G, E94N, L95K, L95M, L95N, L95R, G96H, G96K, G96N, G96R, L97I, L97M, L97V, V98I, V98M, V98T, V98Y, E99K, E99Q, E99R, E99T, V100Y, V100L, V100M, V100I, A101K, A101Y, A101R, A101H, K102E, K102Q, K102R, K102V, I104V, I104Y, I104F, I104L, I104M, V106I, I107C, I107V, G108A, G108C, C109I, C109A, C109M, V110M, V110L, V110I, A111T, A111V, A111Y, A111I, A111S, P112E, T113F, T113S, T113W, T113Y, T114N, N115W, N115R, N115H, N115Y, V117N, V117L, V117T, V117I, I118N, I118A, I118L, I118M, I118S, I118F, I118V, N119Q, N119I, N119L, N119M, N119T, N119S, V120T, V120A, V120P, V120M, M121V, M121A, M121L, M121C, M121I, Q122C, Q122F, Q122R, Q122Y, Q122H, Q122N, Q122S, Q122G, Q122V, Q122W, N123K, N123P, A124V, A124L, A124M, A124I, M125I, M125L, M125A, M125V, C126A, C126P, A127M, L128I, K129A, K129M, C130T, C130S, C130A, G131R, N132C, S133T, S133C, M134I, M134C, M134V, I135V, I135M, V136L, V136M, V136F, V136I, S137R, S137K, P138G, H139S, H139G, P140E, R141Q, R141K, R141Y, R141E, A142T, A142S, K143P, H144L, H144N, H144R, S145T, S145C, S145V, S146T, S146C, S146A, S146N, V147I, V147M, V147A, K148Y, K148L, K148A, K148E, K148H, K148R, T149V, T149I, T149L, T149M, T149F, V150I, V150M, V150G, V150A, E151D, E151K, E151R, E151Q, V152L, V152M, V152I, I153F, I153V, I153C, I153M, N154H, N154A, N154Q, N154K, N154R, E155R, E155K, E155D, E155Q, A156I, L157A, L157I, L157F, A158E, A158V, A158K, A159K, A159Q, A159D, A159E, L160V, L160C, L160A, M1621, M162A, M162F, M162Y, M162L, M162V, K164D, K164A, K164P, N165H, N165G, N165D, L166C, L166A, L166I, L166V, I167V, I167F, I167L, Q168T, Q168N, V169L, V169W, V169C, V169M, V169I, I170L, I170V, T171E, T171P, T171N, T171D, E172D, E172G, E172H, P173G, P173D, S174G, S174D, S174T, M175I, M175H, M175R, E176Q, E176D, L177V, L177T, L177A, L177I, S178V, S178A, S178T, S178G, A179D, A179N, A179E, G180H, G180Q, G180A, G180E, L181I, L181F, M182L, M182H, M182I, M182V, S183Q, S183K, S183D, S183A, S183H, S183E, A184H, A184P, A184S, A184G, V185C, V185I, D186R, L187H, L187K, L187F, L187I, L187V, L187M, C188I, C188L, C188V, I189S, I189F, I189T, I189M, I189V, I189L, C190P, C190A, C190F, T191R, G193S, P194T, P194R, P194K, P194A, G195A, L196M, L196I, L196V, V197I, V197M, V197G, V197A, K198Q, K198E, K198R, A199S, A199V, A200C, A200I, A200V, Y201M, Y201Q, Y201A, S202N, S202K, S202A, S202R, S202T, S202E, S202Q, S203N, S203H, S203A, K205R, K205T, P206V, P206R, P206K, A207V, A207S, A207C, A207T, I208V, I208T, I208Y, I208M, I208L, G209L, G209E, G209M, V210H, V210L, G211L, Q212A, Q212P, Q212G, G213K, N214S, N214K, V215S, V215N, V215A, V215C, V215D, V215P, Q216H, Q216A, Q216P, V217F, V217A, V217I, V217C, V217M, L218I, L218V, L218F, L218Y, V219M, V219F, V219L, V219I, D220C, D220F, R221D, R221P, D222T, D222S, A223C, D224N, L225V, D226E, Q227K, Q227R, Q227M, Q227E, V228A, A229W, A229V, A230R, A230D, A230P, A230Q, A230K, A230E, M231Q, M231R, M231I, M231K, V232I, V232F, V232T, V232C, V232Q, V232M, V232N, I233V, I233L, I233M, K234W, K234Q, K234F, K234A, K234M, G235A, G235S, R236K, R236C, R236W, R236A, R236I, T237F, T237Y, T237C, T237M, F238R, F238M, F238Q, F238Y, F238W, D239R, D239H, D239Q, N240Q, N240A, N240T, N240C, N240S, G241N, L243V, L243M, L243T, L243C, T245M, T245I, T245N, T245S, T245V, T245A, T245C, C246S, C246A, E247G, E247P, E247I, E247A, Q248D, Q248T, Q248E, Q248K, Q248S, Q248N, N249Y, N249R, V250I, V250L, V250M, I251Y, I251F, I251L, I251V, C252V, C252I, P253D, P253Q, P253H, P253V, P253E, E254Q, E254K, E254R, E254D, D255E, K256V, K256M, K256I, K256R, K257A, K257F, K257H, K257Y, E258D, E259A, E259R, E259K, E259D, E259Q, M260I, M260V, M260F, M260L, I261L, I261M, I261V, A262Q, A262D, A262E, A262K, A263E, A263K, A263R, L264M, L264V, L264F, K265Q, K265R, K265A, K265E, K266A, K266E, H267Y, H267R, H267N, H267A, H267Q, G268A, A269S, A269G, A269C, Y270L, Y270F, Y270I, Y270M, Y270W, Y271F, Y271K, Y271V, Y271W, Y271R, Y271H, I272V, I272M, I272L, G273C, N274E, N274D, S275P, S275G, S275A, S275E, E276Q, E276M, E276R, E276D, D277Q, D277E, A278P, A278E, A279E, A279D, A279S, K280Q, K280R, K280T, L281V, L281M, L281I, R282Q, R282K, R282M, R282G, R282E, D283E, D283H, D283N, T284V, T284P, A285L, A285V, A285I, A285M, F286L, F286I, F286W, F286Y, F286V, N288R, N288D, N288E, G289M, G290H, P291K, P291L, P291E, P291T, P291H, P291A, P291N, V292I, V292M, S293A, S293V, S293I, S293N, K294S, K294N, K294P, K294E, K294R, E295R, E295K, E295A, E295Q, E295D, Y296W, Y296R, Y296V, Y296F, Y296H, Y296L, P297A, P297T, P297Q, P297V, P297L, P297H, P297I, G298A, A299K, A299Q, A299R, S300A, S300R, V301I, V301A, K302A, K302R, K302D, K302Q, K302E, K303Q, K303W, K303R, K303E, I304L, I304G, I304Y, A305I, A305V, Q306H, Q306K, Q306E, Q306R, L307I, L307V, L307M, S308A, S308T, I310G, I310V, I310L, I310F, Q311R, Q311T, Q311K, Q311S, Q311D, Q311E, G312A, G312D, G312T, G312K, I313L, I313V, P314A, E315D, D316E, D316G, D316N, A317G, A317T, K318R, K318T, K318Y, V319F, V319L, V319M, V319I, V319Y, I320Y, I320L, V321L, V321P, V321C, V321I, S322A, S322T, S322G, S322V, C323E, C323L, C323I, C323G, C323P, C323V, C323K, T324I, T324V, T324L, R325K, R325E, R325D, R325T, G326D, Y327W, Y327F, Y327V, G328D, G328T, G328S, K329E, K329R, K329P, K329D, K329N, D330E, E331S, E331D, E331H, E331M, E331Y, P332R, P332T, L333M, L333V, L333F, L333I, A334V, A334S, A334M, A334C, K335R, K335Q, K335H, K337Q, K337E, L338F, L338V, L338I, L338M, F339C, F339M, V341I, L342M, L342V, L342I, A343C, A343T, A343V, A343S, A343P, F344I, F344V, F344L, F344M, F345Y, F345V, F345M, F345L, F345I, T346R, T346K, T346P, Y347F, Y347V, D348E, D348K, D348S, K349S, K349G, K349D, K349N, K349T, W350V, W350E, W350I, W350L, W350F, E351D, D352Q, D352H, D352E, I354L, I354V, I354C, H355R, H355A, H355D, H355Q, H355S, H355E, I356M, I356L, I356V, A357C, K358N, K358R, T359A, T359D, T359S, T359N, T359E, N360S, N360T, L361D, L361V, E362L, E362T, E362N, E362S, M363Y, M363E, E364Q, E364Y, E364F, E364H, 1366Y, 1366K, 1366F, 1366A, 1366L, H368L, H368A, S369T, S369A, V370C, V370A, V371M, V371I, V371A, V371W, V371S, V371Y, I372V, I372M, H373W, H373Y, H373F, S374T, N375H, N375Q, N375K, N375T, N375R, T376N, T376S, T376D, P377D, P377I, P377V, P377E, E378K, E378D, E378A, H379N, H379K, H379V, H379R, I380A, I380M, I380V, E381M, E381W, E381K, E381R, E381T, E381H, A382K, A382R, A382E, A382Q, V383M, V383L, V383Y, V383F, V383I, A384S, A384G, E385Q, E385L, E385R, E385K, A386E, A386Q, A386K, A386R, I387M, I387V, I387L, P388R, P388H, P388D, P388Q, P388E, V389A, V389C, V389T, S390T, S390N, S390C, S390G, R391M, R391T, R391I, F392V, F392I, F392M, A393M, A393I, A393W, A393L, A393G, V394I, V394K, Q396R, Q396E, Q396S, Q396D, V397I, V397W, V397A, V397P, V397F, V397Y, G398S, G398C, G398P, G398T, G399A, G399S, T400I, T400S, T400F, T400V, T400H, N401G, N401H, N401D, N401S, N401A, L402N, L402A, L402F, L402M, G403S, A405I, A405F, A405V, A405G, M406Y, M406K, M406F, M406L, M406W, D407H, D407Q, D407Y, D407E, N408T, N408S, N408H, L410F, L410I, L410M, N411T, N411G, N411P, N411A, P412F, P412R, T413A, T414M, T414F, T414V, T415P, T415S, L416M, L416F, L416I, C418F, C418I, C418T, T420S, T420R, W421Y, W421F, N423K, N423R, N423G, N424S, N424R, S425I, S425G, S425W, S425A, I426V, I426T, I426L, S427G, S427T, S427A, E428D, E428H, N429E, F430I, F430Y, F430L, F430V, T431G, T431S, T431N, Y432A, Y432P, Y432F, Y432W, Y433T, Y433R, Y433K, Y433H, Y433M, Y433E, H434R, H434Q, H434D, H434N, L435V, L435F, L435W, L435Y, L435M, M436T, M436L, M436F, M4361, M436V, N437H, N437Q, N437Y, L438V, L438T, L438W, L438C, L438Y, L438I, T439Q, T439S, T439R, T439K, R440K, R440W, V441L, V441I, V441M, S442G, S442A, Y443H, Y443W, Y443R, Y443F, R444E, R444P, R444K, V445I, V445Y, V445F, V445M, V445L, P446E, P446K, D447E, D447N, M448W, M448K, M448P, Y449E, Y449R, Y449F, Y449H, I450M, I450C, I450F, I450V, I450L, P451W, T452S, T452D, T452K, D453E, D453P, D453H, E454A, E454D, E455K, E455Q, E455D, I456F, I456L, I456V, I456W, I456M, W457M, W457Y, W457F, A458H, A458G, E459D, or any combination thereof, in reference to SEQ ID NO: 3.

In some embodiments, the recombinant nucleic acid encodes an engineered ALDH having one or more amino acid alterations comprising: E2Q, E2T, E2P, Q3S, Q3K, A4G, A4S, V5I, V5T, K6F, D7E, D7Q, L9V, K11E, K11D, K11Q, K11R, M12L, M12A, A15R, A15K, S16A, R17K, I18K, 118E, I18Q, Q20F, Q20H, Q21A, Q21R, E22K, E22Q, E22A, F23Y, A24E, A24K, T25A, T25E, Y26L, Y26M, Y26W, P27D, P27T, Q28W, T30D, T30K, T30Q, V31I, V31L, D32N, D32E, K33E, K33Q, K33A, K33R, A34V, V35C, V35M, R36K, T37K, T37A, T37R, V38L, V38I, G39A, K40W, A41L, A41V, I42L, I42V, I42M, I42C, Y43E, Y43L, D44K, D44Q, N45H, N45R, E47D, L48E, L48M, L48D, L49I, H51N, H51E, H51Q, M52L, V54L, D55E, D55R, K58R, N61D, N61E, N61R, Y62F, Y62V, Y62W, A63D, A63E, A63Q, D64H, K65R, I66V, A67E, V70M, V70R, N71D, N71E, N71Y, N71F, N71H, S73T, K74E, K74R, S75G, W77Y, W78E, W78F, W78Y, W78H, W78N, R79D, R79W, M80F, M80V, M80I, K81A, K81R, D82G, K83E, K84R, K84Q, R86V, R86K, I88M, I89V, I89L, K90S, K90D, K90H, K90E, K90R, R91E, R91Q, I92D, P93D, E94G, E94D, L95K, L95M, L95R, G96N, L97I, L97V, L97M, V98Y, V98I, V98M, V98T, E99R, E99T, E99Q, E99K, V100Y, V100I, V100M, V100L, A101Y, A101H, K102R, K102Q, I104F, I104M, I107C, I107V, A111T, A111S, V117I, V117T, V117L, N119T, N119S, V120A, V120T, V120M, M121L, M121V, M121I, Q122H, A124I, A124V, A124L, A124M, M125I, M125L, M125V, C126A, L128I, K129A, K129M, C130A, C130S, S133C, S133T, V136L, V136I, H144R, S145C, S145T, S145V, S146N, V147A, V147I, V147M, K148E, K148L, K148A, K148R, T149M, T149V, T149L, T149I, V150A, V150I, V150M, E151K, E151D, E151Q, V152M, V152I, I153M, I153C, I153F, I153V, N154Q, N154K, E155K, E155R, E155D, E155Q, A156I, L157A, L157F, L157I, A158E, A158V, A158K, A159D, A159K, L160V, L160A, M162F, M162Y, M162V, M162A, M162L, K164A, K164D, K164P, N165D, N165H, L166V, L166C, L166I, I167L, I167V, I167F, Q168T, Q168N, V169C, V169L, V169M, V169I, I170V, T171D, T171E, T171P, T171N, E172D, E172G, E172H, S174T, M175I, M175H, E176D, L177A, L177T, L177I, G180E, L181I, S183Q, S183A, S183H, S183K, A184G, A184S, L187M, L187V, L187I, L187F, L187K, 1189V, 1189L, C190A, P194A, P194T, P194R, P194K, L196V, V197I, K198R, A199V, A200C, Y201A, S202A, S202T, S202R, S203N, S203H, S203A, P206V, A207V, A207T, A207C, I208V, I208M, I208Y, Q212A, Q212P, N214S, V215P, V215N, V215C, V215A, V215S, Q216A, Q216P, V217M, V217A, V217C, L218I, L218V, V2191, V219M, V219L, R221P, R221D, D222T, D222S, A223C, D224N, L225V, Q227M, Q227E, Q227K, V228A, A229V, A230D, A230E, A230P, A230K, M231R, M231Q, M231I, M231K, V232M, I233L, K234F, K234A, K234W, K234M, K234Q, T237F, T237Y, T237C, T237M, F238R, F238Q, F238M, N240T, L243V, T245I, C246S, C246A, Q248N, N249Y, V250M, I251F, I251L, C252V, C252I, P253H, E254K, E254R, E254D, D255E, K256I, K256M, K256V, K256R, K257Y, K257A, E258D, E259R, E259A, E259Q, E259D, M260V, M260L, I261V, I261L, I261M, A262K, A263K, A263R, L264V, L264F, K265R, K265Q, K265E, K266E, H267R, Y270F, Y270L, Y270W, Y270M, Y270I, Y271W, Y271F, Y271K, Y271H, I272V, I272L, I272M, G273C, N274D, N274E, S275A, S275P, S275G, S275E, E276R, E276D, D277E, D277Q, A278E, A279S, A279E, K280T, L281I, L281V, L281M, R282K, R282Q, R282G, R282M, D283E, D283N, D283H, T284V, A285V, F286Y, F286V, F286I, N288E, G290H, P291L, P291A, P291T, P291H, P291N, P291K, S293A, S293I, S293N, K294R, E295D, E295R, E295Q, Y296L, Y296W, A299K, A299Q, A299R, S300A, V301A, K302A, K302E, K302Q, K303E, K303R, I304L, A305V, Q306E, Q306H, Q306K, Q306R, L307I, L307M, L307V, S308T, I310F, I310V, I310L, Q311D, Q311S, Q311R, Q311E, G312A, G312T, G312D, G312K, 1313V, I313L, E315D, D316G, D316E, D316N, A317G, A317T, K318R, V319L, V319M, I320Y, I320L, V321I, V321L, V321C, S322G, S322V, S322A, S322T, C323I, C323V, C323K, C323P, T324I, T324V, T324L, R325E, R325T, G326D, Y327F, Y327W, Y327V, G328S, K329P, K329R, K329D, K329N, D330E, E331D, P332T, P332R, L333I, L333M, L333F, A334S, A334V, A334C, A334M, K335R, F339M, V341I, L342I, L342M, A343S, A343C, A343V, F344M, F344I, F344V, F344L, F345L, F345M, F345Y, F345V, T346P, Y347F, Y347V, D348E, D348K, D348S, K349G, K349T, K349N, K349S, K349D, W350E, W350I, W350V, W350F, W350L, E351D, I354L, I354C, H355D, H355A, H355Q, H355S, H355E, I356L, I356V, K358R, K358N, T359S, T359A, T359N, T359E, N360S, E362S, E362N, E362T, M363E, I366A, I366K, I366Y, I366L, V370C, V370A, V371S, V371A, I372V, I372M, H373F, H373Y, H373W, S374T, N375H, N375T, N375K, N375R, N375Q, T376D, P377E, P377I, P377V, E378K, E378A, E378D, H379R, H379N, H379V, I380V, E381W, E381H, E381M, E381R, E381K, E381T, A382K, V383I, V383M, A384G, A384S, E385R, E385K, A386Q, A386K, A386R, I387M, I387V, I387L, P388R, P388E, P388Q, P388H, P388D, V389T, V389A, V389C, S390G, S390T, S390C, F392M, F392V, F392I, A393G, V394I, Q396E, Q396D, Q396S, V397W, V397I, V397Y, T400S, T400I, T400H, N401D, N401G, N401S, N401H, N401A, L402M, A405V, A405G, M406W, M406F, M406L, D407H, D407Q, L410M, N411G, N411A, N411P, N411T, T414V, T420S, W421F, N423G, N423R, S425I, S425A, S425G, I426V, S427A, S427T, E428D, T431N, Y433T, Y433H, Y433K, Y433M, Y433R, L435M, L435V, M436V, M436F, M4361, N437Q, L438I, L438T, L438Y, L438C, T439S, R440W, R440K, V441M, V441I, V441L, Y443W, Y443F, Y443R, R444K, P446K, D447E, D447N, M448W, M448K, M448P, Y449R, Y449H, I450V, T452S, T452K, D453H, D453P, E454A, E454D, E455K, E455D, W457F, A458H, A458G, or E459D, in reference to SEQ ID NO: 3

In some embodiments, the recombinant nucleic acid encodes an engineered ALDH having one or more amino acid alterations comprising: I42V, N119T or N119S, T149I, A158K, S174T, C246A, E259D, A263R, A299R, Y327V, D348S, V370A, F392I, N401A, or S427T, in reference to SEQ ID NO: 3. In some embodiments, the recombinant nucleic acid encodes an engineered ALDH having an amino acid alteration comprising I42V in reference to SEQ ID NO: 3. In some embodiments, the recombinant nucleic acid encodes an engineered ALDH having an amino acid alteration comprising N119T in reference to SEQ ID NO: 3. In some embodiments, the recombinant nucleic acid encodes an engineered ALDH having an amino acid alteration comprising N119S in reference to SEQ ID NO: 3. In some embodiments, the recombinant nucleic acid encodes an engineered ALDH having an amino acid alteration comprising T149I in reference to SEQ ID NO: 3. In some embodiments, the recombinant nucleic acid encodes an engineered ALDH having an amino acid alteration comprising A158K in reference to SEQ ID NO: 3. In some embodiments, the recombinant nucleic acid encodes an engineered ALDH having an amino acid alteration comprising S174T in reference to SEQ ID NO: 3. In some embodiments, the recombinant nucleic acid encodes an engineered ALDH having an amino acid alteration comprising C246A in reference to SEQ ID NO: 3. In some embodiments, the recombinant nucleic acid encodes an engineered ALDH having an amino acid alteration comprising E259D in reference to SEQ ID NO: 3. In some embodiments, the recombinant nucleic acid encodes an engineered ALDH having an amino acid alteration comprising A263R in reference to SEQ ID NO: 3. In some embodiments, the recombinant nucleic acid encodes an engineered ALDH having an amino acid alteration comprising A299R in reference to SEQ ID NO: 3. In some embodiments, the recombinant nucleic acid encodes an engineered ALDH having an amino acid alteration comprising Y327V in reference to SEQ ID NO: 3. In some embodiments, the recombinant nucleic acid encodes an engineered ALDH having an amino acid alteration comprising D348S in reference to SEQ ID NO: 3. In some embodiments, the recombinant nucleic acid encodes an engineered ALDH having an amino acid alteration comprising V370A in reference to SEQ ID NO: 3. In some embodiments, the recombinant nucleic acid encodes an engineered ALDH having an amino acid alteration comprising F392I in reference to SEQ ID NO: 3. In some embodiments, the recombinant nucleic acid encodes an engineered ALDH having an amino acid alteration comprising N401A in reference to SEQ ID NO: 3. In some embodiments, the recombinant nucleic acid encodes an engineered ALDH having an amino acid alteration comprising S427T in reference to SEQ ID NO: 3.

In some embodiments, provided herein is a recombinant nucleic acid that hybridizes under highly stringent hybridization conditions to an isolated nucleic acid encoding an engineered ALDH described herein. Accordingly, in some embodiments, the recombinant nucleic acid is an isolated nucleic acid that hybridizes under highly stringent hybridization conditions to a nucleic acid that encodes an engineered ALDH that is a variant of a wild-type ALDH (SEQ ID NO: 3), such as an engineered ALDH having one or more alterations at a position described in TABLE 12. In some embodiments, the recombinant nucleic acid molecule is an isolated nucleic acid that hybridizes under highly stringent hybridization conditions to a nucleic acid that encodes an engineered ALDH derived from SEQ ID NO: 3. In some embodiments, the recombinant nucleic acid molecule is an isolated nucleic acid that hybridizes under highly stringent hybridization conditions to a nucleic acid that encodes an engineered ALDH having one or more alterations at a position described in TABLE 12.

In some embodiments, provided herein is a recombinant nucleic acid encoding an engineered ALDH provided herein has an amino acid sequence that is a variant of SEQ ID NO: 3 that includes one or more alterations as described in TABLE 12, wherein the portion, other than the one or more alterations described in TABLE 12, of the engineered ALDH has at least 65%, 70%, 75%, 80%, 85%, 90%, 95%, 96%, 97%, 98% or 99% sequence identity, or is identical, to an amino acid sequence referenced as SEQ ID NO: 3. Accordingly, in some embodiments, a recombinant nucleic acid encodes an engineered ALDH that has an amino acid sequence that includes one or more alterations as described in TABLE 12, other than the alteration described in TABLE 12, of the engineered ALDH has at least 65% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH has at least 70% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH has at least 75% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH has at least 80% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH has at least 85% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH has at least 90% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH has at least 95% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH has at least 96% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH has at least 97% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH has at least 98% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH has at least 99% sequence identity to an amino acid sequence referenced as SEQ ID NO: 3. In other embodiments, a recombinant nucleic acid encodes an engineered ALDH that has an amino acid sequence that includes one or more alterations as described in TABLE 12, other than the alteration described in TABLE 12, of the engineered ALDH is identical to an amino acid sequence referenced as SEQ ID NO: 3. In some embodiments, provided herein is a recombinant nucleic acid that includes a nucleotide sequence encoding an engineered ACL described herein that is operatively linked to a promoter. Such a promoter can express the engineered ACL in a microbial organism as described herein.

In some embodiments, provided herein is a recombinant nucleic acid that includes a nucleotide sequence encoding an engineered ALDH described herein that is operatively linked to a promoter. Such a promoter can express the engineered ALDH in a microbial organism as described herein.

In some embodiments, provided herein is a vector containing a recombinant nucleic acid described herein. In some embodiments, the vector is an expression vector. In some embodiments, the vector comprises double stranded DNA.

A recombinant nucleic acid encoding an engineered ACL described herein also includes a nucleic acid that hybridizes to a nucleic acid disclosed herein or a nucleic acid that hybridizes to a nucleic acid that encodes an amino acid sequence disclosed. Hybridization conditions can include highly stringent, moderately stringent, or low stringency hybridization conditions that are well known to one of skill in the art such as those described herein. Similarly, a recombinant nucleic acid that can be used in the compositions and methods described herein can be described as having a certain percent sequence identity to a nucleic acid disclosed herein or a nucleic acid that hybridizes to a nucleic acid molecule that encodes an amino acid sequence disclosed herein. For example, the nucleic acid can have at least 65%, 70%, 75%, 80%, 85%, 90%, 91%, 92%, 93%, 94%, 95%, 96%, 97%, 98% or 99% sequence identity, or be identical, to a nucleotide described herein.

Stringent hybridization refers to conditions under which hybridized polynucleotides are stable. As known to those of skill in the art, the stability of hybridized polynucleotides is reflected in the melting temperature (Tm) of the hybrids. In general, the stability of hybridized polynucleotides is a function of the salt concentration, for example, the sodium ion concentration, and temperature. A hybridization reaction can be performed under conditions of lower stringency, followed by washes of varying, but higher, stringency. Reference to hybridization stringency relates to such washing conditions. Highly stringent hybridization includes conditions that permit hybridization of only those nucleotide sequences that form stable hybridized polynucleotides in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5×Denhart's solution, 5×SSPE, 0.2% SDS at 42° C., followed by washing in 0.1×SSPE, and 0.1% SDS at 65° C. Hybridization conditions other than highly stringent hybridization conditions can also be used to describe the nucleotide sequences disclosed herein. For example, the phrase moderately stringent hybridization refers to conditions equivalent to hybridization in 50% formamide, 5×Denhart's solution, 5×SSPE, 0.2% SDS at 42° C., followed by washing in 0.2×SSPE, 0.2% SDS, at 42° C. The phrase low stringency hybridization refers to conditions equivalent to hybridization in 10% formamide, 5×Denhart's solution, 6×SSPE, 0.2% SDS at 22° C., followed by washing in 1×SSPE, 0.2% SDS, at 37° C. Denhart's solution contains 1% Ficoll, 1% polyvinylpyrolidone, and 1% bovine serum albumin (BSA). 20×SSPE (sodium chloride, sodium phosphate, ethylene diamine tetraacetic acid (EDTA)) contains 3M sodium chloride, 0.2M sodium phosphate, and 0.025 M (EDTA). Other suitable low, moderate and high stringency hybridization buffers and conditions are well known to those of skill in the art and are described, for example, in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

A recombinant nucleic acid encoding an engineered ACL described herein can have at least a certain sequence identity to a nucleotide sequence disclosed herein. Accordingly, in some aspects described herein, a recombinant nucleic acid encoding an engineered ACL has a nucleotide sequence of at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, or is identical, to a nucleic acid disclosed herein or a nucleic acid that hybridizes to a nucleic acid that encodes an amino acid sequence disclosed herein.

A recombinant nucleic acid encoding an engineered ALDH described herein can have at least a certain sequence identity to a nucleotide sequence disclosed herein. Accordingly, in some aspects described herein, a recombinant nucleic acid encoding an engineered ALDH has a nucleotide sequence of at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, or is identical, to a nucleic acid disclosed herein or a nucleic acid that hybridizes to a nucleic acid that encodes an amino acid sequence disclosed herein.

It is understood that a recombinant nucleic acid described herein or an engineered ACL described here excludes a wild-type parental sequence, for example a parental sequence, such as SEQ ID NO: 1 or 2. It is also understood that a recombinant nucleic acid described herein or an engineered ALDH described here excludes a wild-type parental sequence, for example a parental sequence, such as SEQ ID NO: 3. One skilled in the art will readily understand the meaning of a parental wild-type sequence based on what is well known in the art. It is further understood that such a recombinant nucleic acid described herein can exclude a nucleotide sequence encoding a naturally occurring amino acid sequence as found in nature. Similarly, an engineered ACL described herein can exclude an amino acid sequence as found in nature. Thus, in some embodiments, the recombinant nucleic acid or engineered ACL described herein is as set forth herein, with the proviso that the encoded amino acid sequence is not the wild-type parental sequence or a naturally occurring amino acid sequence and/or that the nucleotide sequence is not a wild-type or naturally occurring nucleotide sequence. A naturally occurring amino acid or nucleotide sequence is understood by those skilled in the art as relating to a sequence that is found in a naturally occurring organism as found in nature. Thus, a nucleotide or amino acid sequence that is not found in the same state or having the same nucleotide or encoded amino acid sequence as in a naturally occurring organism is included within the meaning of a recombinant nucleotide and/or amino acid sequence described herein. For example, a nucleotide or amino acid sequence that has been altered at one or more nucleotide or amino acid positions from a parent sequence, including variants as described herein, are included within the meaning of a nucleotide or amino acid sequence described herein that is not naturally occurring. A recombinant nucleic acid described herein excludes a naturally occurring chromosome that contains the nucleotide sequence, and can further exclude other molecules, as found in a naturally occurring cell, such as DNA binding proteins, for example, proteins such as histones that bind to chromosomes within a eukaryotic cell.

Thus, a recombinant nucleic acid described here has physical and chemical differences compared to a naturally occurring nucleic acid. A recombinant or non-naturally occurring nucleic acid described herein does not contain or does not necessarily have some or all of the chemical bonds, either covalent or non-covalent bonds, of a naturally occurring nucleic acid as found in nature. A recombinant nucleic acid described herein thus differs from a naturally occurring nucleic acid, for example, by having a different chemical structure than a naturally occurring nucleic acid as found in a chromosome. A different chemical structure can occur, for example, by cleavage of phosphodiester bonds that release a recombinant nucleic acid from a naturally occurring chromosome. A recombinant nucleic acid described herein can also differ from a naturally occurring nucleic acid by isolating or separating the nucleic acid from proteins that bind to chromosomal DNA in either prokaryotic or eukaryotic cells, thereby differing from a naturally occurring nucleic acid by different non-covalent bonds. With respect to nucleic acids of prokaryotic origin, a non-naturally occurring nucleic acid described herein does not necessarily have some or all of the naturally occurring chemical bonds of a chromosome, for example, binding to DNA binding proteins such as polymerases or chromosome structural proteins, or is not in a higher order structure such as being supercoiled. With respect to nucleic acids of eukaryotic origin, a non-naturally occurring nucleic acid described herein also does not contain the same internal nucleic acid chemical bonds or chemical bonds with structural proteins as found in chromatin. For example, a non-naturally occurring nucleic acid described herein is not chemically bonded to histones or scaffold proteins and is not contained in a centromere or telomere. Thus, the non-naturally occurring nucleic acids described herein are chemically distinct from a naturally occurring nucleic acid because they either lack or contain different van der Waals interactions, hydrogen bonds, ionic or electrostatic bonds, and/or covalent bonds from a nucleic acid as found in nature. Such differences in bonds can occur either internally within separate regions of the nucleic acid (that is cis) or such difference in bonds can occur in trans, for example, interactions with chromosomal proteins. In the case of a nucleic acid of eukaryotic origin, a cDNA is considered to be a recombinant or non-naturally occurring nucleic acid since the chemical bonds within a cDNA differ from the covalent bonds, that is the sequence, of a gene on chromosomal DNA. Thus, it is understood by those skilled in the art that recombinant or non-naturally occurring nucleic acid is distinct from a naturally occurring nucleic acid.

In some embodiments, provided herein is a method of constructing a host strain that can include, among other steps, introducing a vector disclosed herein into a microbial organism, for example, that is capable of expressing an amino acid sequence encoded by the vector and/or is capable of fermentation. Vectors described herein can be introduced stably or transiently into a microbial organism using techniques well known in the art including, but not limited to, conjugation, electroporation, chemical transformation, transduction, transfection, and ultrasound transformation. Additional methods are disclosed herein, any one of which can be used in the method described herein.

### Microbial Organisms

In some embodiments, provided herein is a microbial organism, in particular a non-naturally occurring microbial organism, that expresses an engineered ACL described herein, that is, an engineered ACL described herein. Thus, provided herein is a non-naturally occurring microbial organism having a recombinant nucleic acid encoding an engineered ACL described herein. Accordingly, in some embodiments, provided herein is microbial organism (e.g., host microbial organism) that has a recombinant nucleic acid encoding an engineered ACL that is a variant of a wild-type or parent ACL (SEQ ID NO: 1 or 2), such as an engineered ACL having one or more alterations at a position described in TABLE 8 or TABLE 9, or a combination of alterations described in TABLE 8 and/or TABLE 9.

In some embodiments, provided herein is microbial organism (e.g., host microbial organism) that has a recombinant nucleic acid encoding an engineered ACL that is a variant of a reference polypeptide, wherein the reference polypeptide has an amino acid sequence of SEQ ID NO: 1, and the engineered ACL has one or more alterations at a position described in TABLE 8 relative to SEQ ID NO: 1. In some embodiments, the microbial organism has a recombinant nucleic acid that encodes an engineered ACL that includes one or more amino acid alterations at a residue corresponding to position 4, 7, 8, 9, 10, 12, 13, 15, 16, 19, 20, 22, 30, 31, 35, 36, 39, 40, 43, 44, 47, 48, 49, 50, 51, 52, 53, 54, 55, 60, 61, 64, 66, 67, 69, 72, 73, 74, 76, 78, 80, 81, 82, 83, 86, 87, 88, 89, 90, 91, 94, 95, 96, 97, 98, 99, 100, 101, 102, 103, 104, 105, 106, 107, 109, 111, 112, 113, 114, 115, 116, 117, 119, 121, 126, 127, 128, 129, 130, 131, 136, 137, 138, 139, 140, 144, 149, 150, 152, 153, 159, 162, 163, 164, 165, 166, 167, 170, 172, 182, 189, 191, 194, 195, 196, 197, 198, 199, 200, 201, 202, 205, 208, 209, 211, 212, 213, 214, 215, 217, 220, 221, 222, 223, 224, 225, 226, 227, 228, 229, 230, 231, 232, 233, 234, 235, 238, 239, 240, 241, 242, 243, 244, 246, 247, 248, 249, 250, 251, 252, 253, 254, 255, 256, 257, 258, 259, 260, 261, 262, 264, 265, 266, 267, 268, 269, 270, 271, 272, 274, 275, 276, 277, 278, 279, 280, 281, 282, 286, 287, 288, 289, 290, 291, 294, 295, 296, 297, 298, 299, 300, 304, 306, 309, 310, 311, 314, 315, 316, 317, 318, 319, 320, 321, 323, 324, 326, 327, 328, 329, 330, 331, 334, 335, 336, 338, 339, 341, 342, 348, 351, 352, 353, 354, 355, 356, 357, 359, 360, 362, 363, 364, 365, 366, 367, 368, 369, 370, 371, 372, 373, 374, 375, 376, 377, 378, 379, 380, 381, 383, 384, 386, 387, 388, 389, 390, 391, 394, 398, 402, 406, 407, 410, 411, 412, 413, 414, 415, 416, 418, 419, 420, 424, 425, 426, 430, 431, 432, 433, 436, 442, 443, 444, 445, 446, 447, 455, 459, 461, 462, 464, 465, 469, 470, 471, 472, 474, 475, 476, 477, 478, 479, 480, 481, 484, 486, 489, 490, 491, 493, 494, 496, 503, 506, 507, 508, 509, 513, 518, 519, 521, 523, 524, 525, 527, 528, 529, 539, 540, 542, 546, 551, 553, 554, 555, 557, 559, 560, 562, 564, 565, 566, 583, or a combination thereof, in SEQ ID NO: 1. In some embodiments, the microbial organism has a recombinant nucleic acid that encodes an engineered ACL that includes one or more amino acid alterations at a residue corresponding to position 7, 8, 10, 12, 15, 16, 19, 52, 54, 64, 66, 67, 73, 74, 82, 87, 90, 94, 100, 111, 115, 117, 119, 121, 129, 131, 138, 139, 140, 144, 150, 165, 166, 172, 182, 191, 199, 209, 212, 217, 221, 223, 224, 227, 229, 230, 231, 232, 233, 234, 235, 238, 240, 243, 249, 253, 254, 256, 258, 259, 262, 268, 271, 275, 277, 281, 288, 290, 296, 297, 300, 304, 306, 316, 318, 320, 321, 323, 328, 330, 331, 336, 352, 353, 363, 364, 365, 366, 369, 372, 374, 376, 378, 388, 390, 394, 398, 406, 407, 416, 432, 443, 445, 455, 462, 469, 470, 471, 480, 481, 484, 486, 490, 491, 493, 494, 496, 508, 513, 518, 519, 521, 523, 525, 546, 554, 555, 560, 565, 583, or a combination thereof, in SEQ ID NO: 1. In some embodiments, the microbial organism has a recombinant nucleic acid that encodes an engineered ACL that includes one or more amino acid alterations at a residue corresponding to position 10, 199, 223, 229, 230, 231, 232, 233, 234, 235, 238, 243, 256, 258, 277, 288, 300, 318, 320, 321, 323, 328, 330, 364, 365, 369, 372, 374, 378, 462, 469, 480, 481, 484, 513, 523, 525, 554, 560, 583, or a combination thereof, in SEQ ID NO: 1. In some embodiments, the microbial organism has a recombinant nucleic acid that encodes an engineered ACL that includes one or more amino acid alterations at a residue corresponding to position 223, 232, 233, 234, 238, 258, 288, 318, 320, 321, 323, 364, 513, 523, or a combination thereof, in SEQ ID NO: 1.

In some embodiments, provided herein is microbial organism (e.g., host microbial organism) that has a recombinant nucleic acid encoding an engineered ACL having one or more alterations described in TABLE 8. Accordingly, in some embodiments, the microbial organism has a recombinant nucleic acid that encodes an engineered ACL having: M at a residue corresponding to position 4 in SEQ ID NO: 1; D, K, Q, S, T at a residue corresponding to position 7 in SEQ ID NO: 1; I, S at a residue corresponding to position 8 in SEQ ID NO: 1; A, K, S at a residue corresponding to position 9 in SEQ ID NO: 1; F, H, P, V, W, Y at a residue corresponding to position 10 in SEQ ID NO: 1; D, E, H, P at a residue corresponding to position 12 in SEQ ID NO: 1; C, M at a residue corresponding to position 13 in SEQ ID NO: 1; D, I, L, P at a residue corresponding to position 15 in SEQ ID NO: 1; E, H, N at a residue corresponding to position 16 in SEQ ID NO: 1; F, L, P, Q at a residue corresponding to position 19 in SEQ ID NO: 1; W at a residue corresponding to position 20 in SEQ ID NO: 1; S at a residue corresponding to position 22 in SEQ ID NO: 1; D at a residue corresponding to position 30 in SEQ ID NO: 1; A at a residue corresponding to position 31 in SEQ ID NO: 1; G, P at a residue corresponding to position 35 in SEQ ID NO: 1; H at a residue corresponding to position 36 in SEQ ID NO: 1; A at a residue corresponding to position 39 in SEQ ID NO: 1; C, F, L, M at a residue corresponding to position 40 in SEQ ID NO: 1; G at a residue corresponding to position 43 in SEQ ID NO: 1; D, H, S, T at a residue corresponding to position 44 in SEQ ID NO: 1; R, RTYTQKEL (SEQ ID NO: 15), RTYTQREL (SEQ ID NO: 16), RTYT (SEQ ID NO: 17) at a residue corresponding to position 47 in SEQ ID NO: 1; a deletion at a residue corresponding to position 48 in SEQ ID NO: 1; a deletion, Y at a residue corresponding to position 49 in SEQ ID NO: 1; a deletion, T at a residue corresponding to position 50 in SEQ ID NO: 1; a deletion, F, H, L, R, W, Y at a residue corresponding to position 51 in SEQ ID NO: 1; a deletion, K, R at a residue corresponding to position 52 in SEQ ID NO: 1; a deletion at a residue corresponding to position 53 in SEQ ID NO: 1; a deletion, L at a residue corresponding to position 54 in SEQ ID NO: 1; L at a residue corresponding to position 55 in SEQ ID NO: 1; R at a residue corresponding to position 60 in SEQ ID NO: 1; F, L at a residue corresponding to position 61 in SEQ ID NO: 1; A, G at a residue corresponding to position 64 in SEQ ID NO: 1; R at a residue corresponding to position 66 in SEQ ID NO: 1; A at a residue corresponding to position 67 in SEQ ID NO: 1; L at a residue corresponding to position 69 in SEQ ID NO: 1; G, T at a residue corresponding to position 72 in SEQ ID NO: 1; F, G, L, V at a residue corresponding to position 73 in SEQ ID NO: 1; K at a residue corresponding to position 74 in SEQ ID NO: 1; T at a residue corresponding to position 76 in SEQ ID NO: 1; V at a residue corresponding to position 78 in SEQ ID NO: 1; G at a residue corresponding to position 80 in SEQ ID NO: 1; PG at a residue corresponding to position 81 in SEQ ID NO: 1; A, E, G, N, R, T at a residue corresponding to position 82 in SEQ ID NO: 1; a deletion, K, P, R, S at a residue corresponding to position 83 in SEQ ID NO: 1; I at a residue corresponding to position 86 in SEQ ID NO: 1; I, V, VAV at a residue corresponding to position 87 in SEQ ID NO: 1; a deletion, A at a residue corresponding to position 88 in SEQ ID NO: 1; a deletion, I, L, V at a residue corresponding to position 89 in SEQ ID NO: 1; C, L, M, V, W at a residue corresponding to position 90 in SEQ ID NO: 1; A, L, M, S at a residue corresponding to position 91 in SEQ ID NO: 1; C, CIEAA (SEQ ID NO: 18), S, T, TIEAA (SEQ ID NO: 19) at a residue corresponding to position 94 in SEQ ID NO: 1; a deletion at a residue corresponding to position 95 in SEQ ID NO: 1; a deletion, E at a residue corresponding to position 96 in SEQ ID NO: 1; a deletion at a residue corresponding to position 97 in SEQ ID NO: 1; a deletion, A, L at a residue corresponding to position 98 in SEQ ID NO: 1; C, M at a residue corresponding to position 99 in SEQ ID NO: 1; S, M, L at a residue corresponding to position 100 in SEQ ID NO: 1; F, M at a residue corresponding to position 101 in SEQ ID NO: 1; H, F, L at a residue corresponding to position 102 in SEQ ID NO: 1; G at a residue corresponding to position 103 in SEQ ID NO: 1; V, L, T at a residue corresponding to position 104 in SEQ ID NO: 1; V, W, H, A, I at a residue corresponding to position 105 in SEQ ID NO: 1; S, Y, R, K at a residue corresponding to position 106 in SEQ ID NO: 1; A, C, V at a residue corresponding to position 107 in SEQ ID NO: 1; L, V, Q at a residue corresponding to position 109 in SEQ ID NO: 1; V, A, M, L, C, VTPAN (SEQ ID NO: 20) at a residue corresponding to position 111 in SEQ ID NO: 1; T, a deletion at a residue corresponding to position 112 in SEQ ID NO: 1; a deletion, L at a residue corresponding to position 113 in SEQ ID NO: 1; a deletion, A, L, V at a residue corresponding to position 114 in SEQ ID NO: 1; a deletion, N at a residue corresponding to position 115 in SEQ ID NO: 1; A, W, P at a residue corresponding to position 116 in SEQ ID NO: 1; S at a residue corresponding to position 117 in SEQ ID NO: 1; T at a residue corresponding to position 119 in SEQ ID NO: 1; R, D at a residue corresponding to position 121 in SEQ ID NO: 1; H at a residue corresponding to position 126 in SEQ ID NO: 1; M, L, MRHTG (SEQ ID NO: 21), MRITG (SEQ ID NO: 22) at a residue corresponding to position 127 in SEQ ID NO: 1; a deletion at a residue corresponding to position 128 in SEQ ID NO: 1; a deletion, I, H at a residue corresponding to position 129 in SEQ ID NO: 1; a deletion at a residue corresponding to position 130 in SEQ ID NO: 1; a deletion, G, D at a residue corresponding to position 131 in SEQ ID NO: 1; L at a residue corresponding to position 136 in SEQ ID NO: 1; T at a residue corresponding to position 137 in SEQ ID NO: 1; D, S at a residue corresponding to position 138 in SEQ ID NO: 1; E at a residue corresponding to position 139 in SEQ ID NO: 1; E, K, D at a residue corresponding to position 140 in SEQ ID NO: 1; T at a residue corresponding to position 144 in SEQ ID NO: 1; R at a residue corresponding to position 149 in SEQ ID NO: 1; I, L at a residue corresponding to position 150 in SEQ ID NO: 1; A, T, C, I at a residue corresponding to position 152 in SEQ ID NO: 1; G, S at a residue corresponding to position 153 in SEQ ID NO: 1; G at a residue corresponding to position 159 in SEQ ID NO: 1; S, H, R, SIDAF (SEQ ID NO: 23), RIESF (SEQ ID NO: 24), HFDSF (SEQ ID NO: 25), HFDAF (SEQ ID NO: 26), HIDSF (SEQ ID NO: 27) at a residue corresponding to position 162 in SEQ ID NO: 1; F, I, a deletion at a residue corresponding to position 163 in SEQ ID NO: 1; a deletion, D at a residue corresponding to position 164 in SEQ ID NO: 1; a deletion, S at a residue corresponding to position 165 in SEQ ID NO: 1; a deletion, F at a residue corresponding to position 166 in SEQ ID NO: 1; L at a residue corresponding to position 167 in SEQ ID NO: 1; G at a residue corresponding to position 170 in SEQ ID NO: 1; L, A, D at a residue corresponding to position 172 in SEQ ID NO: 1; Q at a residue corresponding to position 182 in SEQ ID NO: 1; E, G at a residue corresponding to position 189 in SEQ ID NO: 1; A at a residue corresponding to position 191 in SEQ ID NO: 1; D at a residue corresponding to position 194 in SEQ ID NO: 1; T, P, M at a residue corresponding to position 195 in SEQ ID NO: 1; T, I at a residue corresponding to position 196 in SEQ ID NO: 1; W, C at a residue corresponding to position 197 in SEQ ID NO: 1; Y, V, S, W, I, M, T at a residue corresponding to position 198 in SEQ ID NO: 1; M, L, W, P, F, C, A at a residue corresponding to position 199 in SEQ ID NO: 1; W at a residue corresponding to position 200 in SEQ ID NO: 1; F, T, N, P, Y, L at a residue corresponding to position 201 in SEQ ID NO: 1; T, H, A, Q, E, L, G at a residue corresponding to position 202 in SEQ ID NO: 1; T at a residue corresponding to position 205 in SEQ ID NO: 1; F at a residue corresponding to position 208 in SEQ ID NO: 1; P at a residue corresponding to position 209 in SEQ ID NO: 1; G at a residue corresponding to position 211 in SEQ ID NO: 1; A, C at a residue corresponding to position 212 in SEQ ID NO: 1; M, V, E at a residue corresponding to position 213 in SEQ ID NO: 1; V at a residue corresponding to position 214 in SEQ ID NO: 1; S at a residue corresponding to position 215 in SEQ ID NO: 1; Q, R, Y, H, K, G at a residue corresponding to position 217 in SEQ ID NO: 1; W, M, V at a residue corresponding to position 220 in SEQ ID NO: 1; T, A at a residue corresponding to position 221 in SEQ ID NO: 1; S, N at a residue corresponding to position 222 in SEQ ID NO: 1; A, N, I, L, H at a residue corresponding to position 223 in SEQ ID NO: 1; T, Q, H, I, N, M at a residue corresponding to position 224 in SEQ ID NO: 1; L, A, F, Q at a residue corresponding to position 225 in SEQ ID NO: 1; Y, C, T, W at a residue corresponding to position 226 in SEQ ID NO: 1; G, E, M, T, A, GKWSQD (SEQ ID NO: 28), GKWSQSY (SEQ ID NO: 29), GKWSQ (SEQ ID NO: 30), GKWSQDLK (SEQ IDNO: 31), GKWSQQLQ (SEQ ID NO: 32) at a residue corresponding to position 227 in SEQ ID NO: 1; a deletion at a residue corresponding to position 228 in SEQ ID NO: 1; a deletion, F, S, L, G, W, I, M, H at a residue corresponding to position 229 in SEQ ID NO: 1; a deletion, R, M, P, T, E, Q, A at a residue corresponding to position 230 in SEQ ID NO: 1; a deletion, Q, P, H, QDLQ (SEQ ID NO: 33) at a residue corresponding to position 231 in SEQ ID NO: 1; a deletion, W, A, Y, N, K, Q, D at a residue corresponding to position 232 in SEQ ID NO: 1; a deletion, W, R, M, Y, P at a residue corresponding to position 233 in SEQ ID NO: 1; a deletion, F, Y, T, K, R, Q, P, G, H, E at a residue corresponding to position 234 in SEQ ID NO: 1; H, S, R, K, M, Q, D, G at a residue corresponding to position 235 in SEQ ID NO: 1; P, N, H, T, D, DISAC (SEQ ID NO: 34), DVS at a residue corresponding to position 238 in SEQ ID NO: 1; V, F, R, a deletion at a residue corresponding to position 239 in SEQ ID NO: 1; a deletion, F, H, T, W, S, L, C, V at a residue corresponding to position 240 in SEQ ID NO: 1; a deletion, C, M, P, I at a residue corresponding to position 241 in SEQ ID NO: 1; a deletion, G, V, C at a residue corresponding to position 242 in SEQ ID NO: 1; L, C, M at a residue corresponding to position 243 in SEQ ID NO: 1; M, V at a residue corresponding to position 244 in SEQ ID NO: 1; W, F, L, Y at a residue corresponding to position 246 in SEQ ID NO: 1; F, C at a residue corresponding to position 247 in SEQ ID NO: 1; S, P at a residue corresponding to position 248 in SEQ ID NO: 1; A, V, M at a residue corresponding to position 249 in SEQ ID NO: 1; M, A, W at a residue corresponding to position 250 in SEQ ID NO: 1; A, H at a residue corresponding to position 251 in SEQ ID NO: 1; I, M, W, Q at a residue corresponding to position 252 in SEQ ID NO: 1; T, Q, N, I, H, V, M at a residue corresponding to position 253 in SEQ ID NO: 1; E, I, T, Y, H, V, L, P, A, M, F, S, G at a residue corresponding to position 254 in SEQ ID NO: 1; M, T, I, N, Y, C, ILLANV (SEQ ID NO: 35), ILLANVHY (SEQ ID NO: 36), ILLLNVHY (SEQ ID NO: 37), ILLLNV (SEQ ID NO: 38), ILLLNVHK (SEQ ID NO: 39), ILLHNVHK (SEQ ID NO: 40), ILLSNVHY (SEQ ID NO: 41) at a residue corresponding to position 255 in SEQ ID NO: 1; I, V, H, C, F, a deletion at a residue corresponding to position 256 in SEQ ID NO: 1; a deletion, L, T, N at a residue corresponding to position 257 in SEQ ID NO: 1; a deletion, S, L, P, M, H, T, A at a residue corresponding to position 258 in SEQ ID NO: 1; a deletion, C, A at a residue corresponding to position 259 in SEQ ID NO: 1; a deletion, F, L, M, V at a residue corresponding to position 260 in SEQ ID NO: 1; a deletion, Y, M, W, A, F, L, S at a residue corresponding to position 261 in SEQ ID NO: 1; a deletion, M, K, Y, R, G, C, L, V, S, N at a residue corresponding to position 262 in SEQ ID NO: 1; G, W, I, S, V at a residue corresponding to position 264 in SEQ ID NO: 1; R, C, H, K at a residue corresponding to position 265 in SEQ ID NO: 1; I, V, Q, C at a residue corresponding to position 266 in SEQ ID NO: 1; Y at a residue corresponding to position 267 in SEQ ID NO: 1; V, M, T, F, IMPR (SEQ ID NO: 42), EIMNR (SEQ ID NO: 43) at a residue corresponding to position 268 in SEQ ID NO: 1; M, at a residue corresponding to position 269 in SEQ ID NO: 1; a deletion, P at a residue corresponding to position 270 in SEQ ID NO: 1; a deletion, R, Q at a residue corresponding to position 271 in SEQ ID NO: 1; W at a residue corresponding to position 272 in SEQ ID NO: 1; P, PEEMLRAIQ (SEQ ID NO: 44), PEEMLQAIQ (SEQ ID NO: 45) at a residue corresponding to position 274 in SEQ ID NO: 1; D, A, E, R, K, EEMLQAIQ (SEQ ID NO: 46), EEMLRAIQ (SEQ ID NO: 47), a deletion, KEMLQAIQ (SEQ ID NO: 48) at a residue corresponding to position 275 in SEQ ID NO: 1; a deletion at a residue corresponding to position 276 in SEQ ID NO: 1; a deletion, M, Y, L, F at a residue corresponding to position 277 in SEQ ID NO: 1; a deletion at a residue corresponding to position 278 in SEQ ID NO: 1; a deletion, Q, R, QAIQ (SEQ ID NO: 49), RAIQ (SEQ ID NO: 50) at a residue corresponding to position 279 in SEQ ID NO: 1; a deletion at a residue corresponding to position 280 in SEQ ID NO: 1; a deletion, V, M, L, I at a residue corresponding to position 281 in SEQ ID NO: 1; a deletion, Q at a residue corresponding to position 282 in SEQ ID NO: 1; P, C at a residue corresponding to position 286 in SEQ ID NO: 1; T, TVFFA (SEQ ID NO: 51), TSFFA (SEQ ID NO: 52), TFFFA (SEQ ID NO: 53), TMFFA (SEQ ID NO: 54), TVFHA (SEQ ID NO: 55) at a residue corresponding to position 287 in SEQ ID NO: 1; H, F, M, Y, R, I, V, T, C, a deletion at a residue corresponding to position 288 in SEQ ID NO: 1; a deletion, M, V, A, L, F, I at a residue corresponding to position 289 in SEQ ID NO: 1; a deletion, A, W, H, C, F, V, P, L at a residue corresponding to position 290 in SEQ ID NO: 1; a deletion, A, M, G, V at a residue corresponding to position 291 in SEQ ID NO: 1; A, T at a residue corresponding to position 294 in SEQ ID NO: 1; L, a deletion at a residue corresponding to position 295 in SEQ ID NO: 1; M, L at a residue corresponding to position 296 in SEQ ID NO: 1; M, Q, I, A, L, T at a residue corresponding to position 297 in SEQ ID NO: 1; M, A, F, R, IM at a residue corresponding to position 298 in SEQ ID NO: 1; M, F at a residue corresponding to position 299 in SEQ ID NO: 1; A at a residue corresponding to position 300 in SEQ ID NO: 1; M, R, E at a residue corresponding to position 304 in SEQ ID NO: 1; D at a residue corresponding to position 306 in SEQ ID NO: 1; L at a residue corresponding to position 309 in SEQ ID NO: 1; L, D at a residue corresponding to position 310 in SEQ ID NO: 1; L at a residue corresponding to position 311 in SEQ ID NO: 1; S, SHVRSV (SEQ ID NO: 56), SHVRTV (SEQ ID NO: 57) at a residue corresponding to position 314 in SEQ ID NO: 1; S, a deletion at a residue corresponding to position 315 in SEQ ID NO: 1; a deletion, V, L, I at a residue corresponding to position 316 in SEQ ID NO: 1; a deletion, H, N, Q, T at a residue corresponding to position 317 in SEQ ID NO: 1; a deletion, Y, M, T, S, C, Q, R, H, V, W, I, L at a residue corresponding to position 318 in SEQ ID NO: 1; a deletion, C, F, V, L, M at a residue corresponding to position 319 in SEQ ID NO: 1; F, I, V, Y, T, M, W at a residue corresponding to position 320 in SEQ ID NO: 1; C at a residue corresponding to position 321 in SEQ ID NO: 1; G at a residue corresponding to position 323 in SEQ ID NO: 1; S, M at a residue corresponding to position 324 in SEQ ID NO: 1; V, I, C at a residue corresponding to position 326 in SEQ ID NO: 1; S at a residue corresponding to position 327 in SEQ ID NO: 1; H, V, R, S, RELE (SEQ ID NO: 58) at a residue corresponding to position 328 in SEQ ID NO: 1; E, a deletion, ELE at a residue corresponding to position 329 in SEQ ID NO: 1; a deletion, L, T, I at a residue corresponding to position 330 in SEQ ID NO: 1; a deletion, M, E, C, L, I at a residue corresponding to position 331 in SEQ ID NO: 1; A, W, C, M, I at a residue corresponding to position 334 in SEQ ID NO: 1; Q, R at a residue corresponding to position 335 in SEQ ID NO: 1; K at a residue corresponding to position 336 in SEQ ID NO: 1; M at a residue corresponding to position 338 in SEQ ID NO: 1; S at a residue corresponding to position 339 in SEQ ID NO: 1; H, T at a residue corresponding to position 341 in SEQ ID NO: 1; L, F at a residue corresponding to position 342 in SEQ ID NO: 1; D, H, Y at a residue corresponding to position 348 in SEQ ID NO: 1; V, C, T, F, A, W at a residue corresponding to position 351 in SEQ ID NO: 1; E, K, D at a residue corresponding to position 352 in SEQ ID NO: 1; V, L, M at a residue corresponding to position 353 in SEQ ID NO: 1; C, F, L, Y, A, K, S, I at a residue corresponding to position 354 in SEQ ID NO: 1; E at a residue corresponding to position 355 in SEQ ID NO: 1; A at a residue corresponding to position 356 in SEQ ID NO: 1; W at a residue corresponding to position 357 in SEQ ID NO: 1; M at a residue corresponding to position 359 in SEQ ID NO: 1; S at a residue corresponding to position 360 in SEQ ID NO: 1; C, S at a residue corresponding to position 362 in SEQ ID NO: 1; A, T, G at a residue corresponding to position 363 in SEQ ID NO: 1; C, S at a residue corresponding to position 364 in SEQ ID NO: 1; L, I, M, C at a residue corresponding to position 365 in SEQ ID NO: 1; S, V, T at a residue corresponding to position 366 in SEQ ID NO: 1; A, C, S, H at a residue corresponding to position 367 in SEQ ID NO: 1; H at a residue corresponding to position 368 in SEQ ID NO: 1; H, Y, I, C, L, V, W, S, T, M at a residue corresponding to position 369 in SEQ ID NO: 1; Y, R, T, S, H, G, N, Q at a residue corresponding to position 370 in SEQ ID NO: 1; W at a residue corresponding to position 371 in SEQ ID NO: 1; K, W, Q, H, S, R, N at a residue corresponding to position 372 in SEQ ID NO: 1; S, H, P, Q, E at a residue corresponding to position 373 in SEQ ID NO: 1; D, T, A, R, S, P, W, E, Q, H at a residue corresponding to position 374 in SEQ ID NO: 1; P at a residue corresponding to position 375 in SEQ ID NO: 1; T, K, E, Q, H, D at a residue corresponding to position 376 in SEQ ID NO: 1; Y, R, N at a residue corresponding to position 377 in SEQ ID NO: 1; S, Y, P, V, A at a residue corresponding to position 378 in SEQ ID NO: 1; G at a residue corresponding to position 379 in SEQ ID NO: 1; T at a residue corresponding to position 380 in SEQ ID NO: 1; V, S, I at a residue corresponding to position 381 in SEQ ID NO: 1; Q, H, T, Y, W, P at a residue corresponding to position 383 in SEQ ID NO: 1; I, P, A, M at a residue corresponding to position 384 in SEQ ID NO: 1; S, A at a residue corresponding to position 386 in SEQ ID NO: 1; G, S, H at a residue corresponding to position 387 in SEQ ID NO: 1; T, M, V, L at a residue corresponding to position 388 in SEQ ID NO: 1; R, K, E at a residue corresponding to position 389 in SEQ ID NO: 1; C, L, A at a residue corresponding to position 390 in SEQ ID NO: 1; K at a residue corresponding to position 391 in SEQ ID NO: 1; D at a residue corresponding to position 394 in SEQ ID NO: 1; D, N at a residue corresponding to position 398 in SEQ ID NO: 1; V, L, I at a residue corresponding to position 402 in SEQ ID NO: 1; L, V at a residue corresponding to position 406 in SEQ ID NO: 1; D at a residue corresponding to position 407 in SEQ ID NO: 1; N, Q, NTENAPR (SEQ ID NO: 59), NTENAPD (SEQ ID NO: 60) at a residue corresponding to position 410 in SEQ ID NO: 1; T, TENAPD (SEQ ID NO: 61), TQNAPR (SEQ ID NO: 62), a deletion, TENAPR (SEQ ID NO: 63), TQNAPN (SEQ ID NO: 64) at a residue corresponding to position 411 in SEQ ID NO: 1; a deletion, Q at a residue corresponding to position 412 in SEQ ID NO: 1; a deletion, N, NNPN (SEQ ID NO: 65) at a residue corresponding to position 413 in SEQ ID NO: 1; a deletion, A, S, N at a residue corresponding to position 414 in SEQ ID NO: 1; a deletion, P at a residue corresponding to position 415 in SEQ ID NO: 1; a deletion, D, R, N, E at a residue corresponding to position 416 in SEQ ID NO: 1; P at a residue corresponding to position 418 in SEQ ID NO: 1; G at a residue corresponding to position 419 in SEQ ID NO: 1; E at a residue corresponding to position 420 in SEQ ID NO: 1; M, V at a residue corresponding to position 424 in SEQ ID NO: 1; V, L at a residue corresponding to position 425 in SEQ ID NO: 1; M, L at a residue corresponding to position 426 in SEQ ID NO: 1; T, N, S at a residue corresponding to position 430 in SEQ ID NO: 1; I, L at a residue corresponding to position 431 in SEQ ID NO: 1; T, F at a residue corresponding to position 432 in SEQ ID NO: 1; S, K at a residue corresponding to position 433 in SEQ ID NO: 1; W at a residue corresponding to position 436 in SEQ ID NO: 1; T at a residue corresponding to position 442 in SEQ ID NO: 1; R, K, RASFA (SEQ ID NO: 66) at a residue corresponding to position 443 in SEQ ID NO: 1; a deletion at a residue corresponding to position 444 in SEQ ID NO: 1; a deletion, M, T, S, A at a residue corresponding to position 445 in SEQ ID NO: 1; a deletion at a residue corresponding to position 446 in SEQ ID NO: 1; a deletion, A at a residue corresponding to position 447 in SEQ ID NO: 1; S, G at a residue corresponding to position 455 in SEQ ID NO: 1; G, E, D at a residue corresponding to position 459 in SEQ ID NO: 1; D, G at a residue corresponding to position 461 in SEQ ID NO: 1; R, G at a residue corresponding to position 462 in SEQ ID NO: 1; L, Y at a residue corresponding to position 464 in SEQ ID NO: 1; H at a residue corresponding to position 465 in SEQ ID NO: 1; M at a residue corresponding to position 469 in SEQ ID NO: 1; C, G at a residue corresponding to position 470 in SEQ ID NO: 1; Y, K, M, W, I, R at a residue corresponding to position 471 in SEQ ID NO: 1; I, F, C, Y, L, M at a residue corresponding to position 472 in SEQ ID NO: 1; E, EDGH (SEQ ID NO: 67), EDGY (SEQ ID NO: 68) at a residue corresponding to position 474 in SEQ ID NO: 1; D, a deletion at a residue corresponding to position 475 in SEQ ID NO: 1; a deletion at a residue corresponding to position 476 in SEQ ID NO: 1; a deletion, H, F, N, E, Y at a residue corresponding to position 477 in SEQ ID NO: 1; W, I, M at a residue corresponding to position 478 in SEQ ID NO: 1; T, H, Y, F, W at a residue corresponding to position 479 in SEQ ID NO: 1; V, L at a residue corresponding to position 480 in SEQ ID NO: 1; C, I, T at a residue corresponding to position 481 in SEQ ID NO: 1; L at a residue corresponding to position 484 in SEQ ID NO: 1; D at a residue corresponding to position 486 in SEQ ID NO: 1; I at a residue corresponding to position 489 in SEQ ID NO: 1; R, C, V at a residue corresponding to position 490 in SEQ ID NO: 1; G, R, N at a residue corresponding to position 491 in SEQ ID NO: 1; F, H, Y, L at a residue corresponding to position 493 in SEQ ID NO: 1; N at a residue corresponding to position 494 in SEQ ID NO: 1; F, S, Y at a residue corresponding to position 496 in SEQ ID NO: 1; L, V, I at a residue corresponding to position 503 in SEQ ID NO: 1; R, RHPD (SEQ ID NO: 69) at a residue corresponding to position 506 in SEQ ID NO: 1; a deletion at a residue corresponding to position 507 in SEQ ID NO: 1; a deletion, P at a residue corresponding to position 508 in SEQ ID NO: 1; a deletion, D at a residue corresponding to position 509 in SEQ ID NO: 1; A, V, C at a residue corresponding to position 513 in SEQ ID NO: 1; V at a residue corresponding to position 518 in SEQ ID NO: 1; P at a residue corresponding to position 519 in SEQ ID NO: 1; E, P at a residue corresponding to position 521 in SEQ ID NO: 1; Q, A, R, M, Y, D at a residue corresponding to position 523 in SEQ ID NO: 1; S, A at a residue corresponding to position 524 in SEQ ID NO: 1; S, T, D, G, E at a residue corresponding to position 525 in SEQ ID NO: 1; P, T, V at a residue corresponding to position 527 in SEQ ID NO: 1; A, V at a residue corresponding to position 528 in SEQ ID NO: 1; V, A at a residue corresponding to position 529 in SEQ ID NO: 1; L, V at a residue corresponding to position 539 in SEQ ID NO: 1; T at a residue corresponding to position 540 in SEQ ID NO: 1; E, D at a residue corresponding to position 542 in SEQ ID NO: 1; A, K at a residue corresponding to position 546 in SEQ ID NO: 1; K, R at a residue corresponding to position 551 in SEQ ID NO: 1; S at a residue corresponding to position 553 in SEQ ID NO: 1; S, P, H at a residue corresponding to position 554 in SEQ ID NO: 1; F at a residue corresponding to position 555 in SEQ ID NO: 1; K at a residue corresponding to position 557 in SEQ ID NO: 1; P at a residue corresponding to position 559 in SEQ ID NO: 1; G at a residue corresponding to position 560 in SEQ ID NO: 1; V, a deletion at a residue corresponding to position 562 in SEQ ID NO: 1; F at a residue corresponding to position 564 in SEQ ID NO: 1; V, F, FV at a residue corresponding to position 565 in SEQ ID NO: 1; E, VE at a residue corresponding to position 566 in SEQ ID NO: 1; Q, E at a residue corresponding to position 583 in SEQ ID NO: 1; more than one of the foregoing; or any combination thereof.

In some embodiments, provided herein is microbial organism (e.g., host microbial organism) that has a recombinant nucleic acid encoding an engineered ACL having: K, S at a residue corresponding to position 7 in SEQ ID NO: 1; S at a residue corresponding to position 8 in SEQ ID NO: 1; W, Y, F at a residue corresponding to position 10 in SEQ ID NO: 1; E, P, D at a residue corresponding to position 12 in SEQ ID NO: 1; I at a residue corresponding to position 15 in SEQ ID NO: 1; E at a residue corresponding to position 16 in SEQ ID NO: 1; F at a residue corresponding to position 19 in SEQ ID NO: 1; R, K at a residue corresponding to position 52 in SEQ ID NO: 1; L at a residue corresponding to position 54 in SEQ ID NO: 1; A at a residue corresponding to position 64 in SEQ ID NO: 1; R at a residue corresponding to position 66 in SEQ ID NO: 1; A at a residue corresponding to position 67 in SEQ ID NO: 1; L at a residue corresponding to position 73 in SEQ ID NO: 1; K at a residue corresponding to position 74 in SEQ ID NO: 1; T at a residue corresponding to position 82 in SEQ ID NO: 1; I at a residue corresponding to position 87 in SEQ ID NO: 1; L, M at a residue corresponding to position 90 in SEQ ID NO: 1; T at a residue corresponding to position 94 in SEQ ID NO: 1; L at a residue corresponding to position 100 in SEQ ID NO: 1; V at a residue corresponding to position 111 in SEQ ID NO: 1; N at a residue corresponding to position 115 in SEQ ID NO: 1; S at a residue corresponding to position 117 in SEQ ID NO: 1; T at a residue corresponding to position 119 in SEQ ID NO: 1; D at a residue corresponding to position 121 in SEQ ID NO: 1; I at a residue corresponding to position 129 in SEQ ID NO: 1; G at a residue corresponding to position 131 in SEQ ID NO: 1; D, S at a residue corresponding to position 138 in SEQ ID NO: 1; E at a residue corresponding to position 139 in SEQ ID NO: 1; K, E, D at a residue corresponding to position 140 in SEQ ID NO: 1; T at a residue corresponding to position 144 in SEQ ID NO: 1; L, I at a residue corresponding to position 150 in SEQ ID NO: 1; S at a residue corresponding to position 165 in SEQ ID NO: 1; F at a residue corresponding to position 166 in SEQ ID NO: 1; L, A, D at a residue corresponding to position 172 in SEQ ID NO: 1; Q at a residue corresponding to position 182 in SEQ ID NO: 1; A at a residue corresponding to position 191 in SEQ ID NO: 1; A at a residue corresponding to position 199 in SEQ ID NO: 1; P at a residue corresponding to position 209 in SEQ ID NO: 1; C at a residue corresponding to position 212 in SEQ ID NO: 1; R at a residue corresponding to position 217 in SEQ ID NO: 1; T, A at a residue corresponding to position 221 in SEQ ID NO: 1; N, I, A, L, H at a residue corresponding to position 223 in SEQ ID NO: 1; Q at a residue corresponding to position 224 in SEQ ID NO: 1; E, T, A at a residue corresponding to position 227 in SEQ ID NO: 1; I, L, M at a residue corresponding to position 229 in SEQ ID NO: 1; Q, A at a residue corresponding to position 230 in SEQ ID NO: 1; H, Q, P at a residue corresponding to position 231 in SEQ ID NO: 1; Y, K, Q, N at a residue corresponding to position 232 in SEQ ID NO: 1; W, Y at a residue corresponding to position 233 in SEQ ID NO: 1; Y, F, H at a residue corresponding to position 234 in SEQ ID NO: 1; S at a residue corresponding to position 235 in SEQ ID NO: 1; D, H, T at a residue corresponding to position 238 in SEQ ID NO: 1; T, H, C, S at a residue corresponding to position 240 in SEQ ID NO: 1; C, M at a residue corresponding to position 243 in SEQ ID NO: 1; M, V at a residue corresponding to position 249 in SEQ ID NO: 1; H, M, Q at a residue corresponding to position 253 in SEQ ID NO: 1; M at a residue corresponding to position 254 in SEQ ID NO: 1; I, V at a residue corresponding to position 256 in SEQ ID NO: 1; T, H, A, L, M at a residue corresponding to position 258 in SEQ ID NO: 1; A at a residue corresponding to position 259 in SEQ ID NO: 1; K, C, N at a residue corresponding to position 262 in SEQ ID NO: 1; F at a residue corresponding to position 268 in SEQ ID NO: 1; R, Q at a residue corresponding to position 271 in SEQ ID NO: 1; E at a residue corresponding to position 275 in SEQ ID NO: 1; L, M at a residue corresponding to position 277 in SEQ ID NO: 1; V at a residue corresponding to position 281 in SEQ ID NO: 1; V, I, F, T, H, Y at a residue corresponding to position 288 in SEQ ID NO: 1; V, A at a residue corresponding to position 290 in SEQ ID NO: 1; L, M at a residue corresponding to position 296 in SEQ ID NO: 1; I at a residue corresponding to position 297 in SEQ ID NO: 1; A at a residue corresponding to position 300 in SEQ ID NO: 1; E at a residue corresponding to position 304 in SEQ ID NO: 1; D at a residue corresponding to position 306 in SEQ ID NO: 1; L at a residue corresponding to position 316 in SEQ ID NO: 1; S, R, H, W, Y, M, T, C, Q, V, I, L at a residue corresponding to position 318 in SEQ ID NO: 1; M, F, T, Y, W at a residue corresponding to position 320 in SEQ ID NO: 1; C at a residue corresponding to position 321 in SEQ ID NO: 1; G at a residue corresponding to position 323 in SEQ ID NO: 1; V, H, R at a residue corresponding to position 328 in SEQ ID NO: 1; I, L at a residue corresponding to position 330 in SEQ ID NO: 1; I at a residue corresponding to position 331 in SEQ ID NO: 1; K at a residue corresponding to position 336 in SEQ ID NO: 1; K at a residue corresponding to position 352 in SEQ ID NO: 1; V, L at a residue corresponding to position 353 in SEQ ID NO: 1; G at a residue corresponding to position 363 in SEQ ID NO: 1; S at a residue corresponding to position 364 in SEQ ID NO: 1; C, L, I, M at a residue corresponding to position 365 in SEQ ID NO: 1; V at a residue corresponding to position 366 in SEQ ID NO: 1; H, S, T, M at a residue corresponding to position 369 in SEQ ID NO: 1; S, K, H, N at a residue corresponding to position 372 in SEQ ID NO: 1; Q, R, T, P, H, S, A, D, E at a residue corresponding to position 374 in SEQ ID NO: 1; T at a residue corresponding to position 376 in SEQ ID NO: 1; A, P, S, V at a residue corresponding to position 378 in SEQ ID NO: 1; T at a residue corresponding to position 388 in SEQ ID NO: 1; C, A at a residue corresponding to position 390 in SEQ ID NO: 1; D at a residue corresponding to position 394 in SEQ ID NO: 1; N at a residue corresponding to position 398 in SEQ ID NO: 1; L at a residue corresponding to position 406 in SEQ ID NO: 1; D at a residue corresponding to position 407 in SEQ ID NO: 1; N, E at a residue corresponding to position 416 in SEQ ID NO: 1; F at a residue corresponding to position 432 in SEQ ID NO: 1; R, K at a residue corresponding to position 443 in SEQ ID NO: 1; A at a residue corresponding to position 445 in SEQ ID NO: 1; S, G at a residue corresponding to position 455 in SEQ ID NO: 1; R at a residue corresponding to position 462 in SEQ ID NO: 1; M at a residue corresponding to position 469 in SEQ ID NO: 1; G at a residue corresponding to position 470 in SEQ ID NO: 1; R, M, W at a residue corresponding to position 471 in SEQ ID NO: 1; V at a residue corresponding to position 480 in SEQ ID NO: 1; I, C at a residue corresponding to position 481 in SEQ ID NO: 1; L at a residue corresponding to position 484 in SEQ ID NO: 1; D at a residue corresponding to position 486 in SEQ ID NO: 1; C at a residue corresponding to position 490 in SEQ ID NO: 1; R, G at a residue corresponding to position 491 in SEQ ID NO: 1; L, H, Y at a residue corresponding to position 493 in SEQ ID NO: 1; N at a residue corresponding to position 494 in SEQ ID NO: 1; Y at a residue corresponding to position 496 in SEQ ID NO: 1; P at a residue corresponding to position 508 in SEQ ID NO: 1; A, V, C at a residue corresponding to position 513 in SEQ ID NO: 1; V at a residue corresponding to position 518 in SEQ ID NO: 1; P at a residue corresponding to position 519 in SEQ ID NO: 1; E, P at a residue corresponding to position 521 in SEQ ID NO: 1; R, Q, A, Y, M, D at a residue corresponding to position 523 in SEQ ID NO: 1; D, S, T, G, E at a residue corresponding to position 525 in SEQ ID NO: 1; K at a residue corresponding to position 546 in SEQ ID NO: 1; H, S, P at a residue corresponding to position 554 in SEQ ID NO: 1; F at a residue corresponding to position 555 in SEQ ID NO: 1; G at a residue corresponding to position 560 in SEQ ID NO: 1; V at a residue corresponding to position 565 in SEQ ID NO: 1; Q, E at a residue corresponding to position 583 in SEQ ID NO: 1; more than one of the foregoing; or any combination thereof.

In some embodiments, provided herein is microbial organism (e.g., host microbial organism) that has a recombinant nucleic acid encoding an engineered ACL having: F at a residue corresponding to position 10 in SEQ ID NO: 1; A at a residue corresponding to position 199 in SEQ ID NO: 1; L, H, A at a residue corresponding to position 223 in SEQ ID NO: 1; M at a residue corresponding to position 229 in SEQ ID NO: 1; Q, A at a residue corresponding to position 230 in SEQ ID NO: 1; P at a residue corresponding to position 231 in SEQ ID NO: 1; N at a residue corresponding to position 232 in SEQ ID NO: 1; W, Y at a residue corresponding to position 233 in SEQ ID NO: 1; H at a residue corresponding to position 234 in SEQ ID NO: 1; S at a residue corresponding to position 235 in SEQ ID NO: 1; T at a residue corresponding to position 238 in SEQ ID NO: 1; C, M at a residue corresponding to position 243 in SEQ ID NO: 1; I, V at a residue corresponding to position 256 in SEQ ID NO: 1; L, M at a residue corresponding to position 258 in SEQ ID NO: 1; M at a residue corresponding to position 277 in SEQ ID NO: 1; Y, H at a residue corresponding to position 288 in SEQ ID NO: 1; A at a residue corresponding to position 300 in SEQ ID NO: 1; V, T, C, I, L, Q, M, Y at a residue corresponding to position 318 in SEQ ID NO: 1; W, Y at a residue corresponding to position 320 in SEQ ID NO: 1; C at a residue corresponding to position 321 in SEQ ID NO: 1; G at a residue corresponding to position 323 in SEQ ID NO: 1; R at a residue corresponding to position 328 in SEQ ID NO: 1; L at a residue corresponding to position 330 in SEQ ID NO: 1; S at a residue corresponding to position 364 in SEQ ID NO: 1; I, M at a residue corresponding to position 365 in SEQ ID NO: 1; M at a residue corresponding to position 369 in SEQ ID NO: 1; N at a residue corresponding to position 372 in SEQ ID NO: 1; E at a residue corresponding to position 374 in SEQ ID NO: 1; V at a residue corresponding to position 378 in SEQ ID NO: 1; R at a residue corresponding to position 462 in SEQ ID NO: 1; M at a residue corresponding to position 469 in SEQ ID NO: 1; V at a residue corresponding to position 480 in SEQ ID NO: 1; C at a residue corresponding to position 481 in SEQ ID NO: 1; L at a residue corresponding to position 484 in SEQ ID NO: 1; C, A, V at a residue corresponding to position 513 in SEQ ID NO: 1; D at a residue corresponding to position 523 in SEQ ID NO: 1; G, E, T at a residue corresponding to position 525 in SEQ ID NO: 1; S, P at a residue corresponding to position 554 in SEQ ID NO: 1; G at a residue corresponding to position 560 in SEQ ID NO: 1; or E at a residue corresponding to position 583 in SEQ ID NO: 1; or any combination thereof.

In some embodiments, provided herein is microbial organism (e.g., host microbial organism) that has a recombinant nucleic acid encoding an engineered ACL having: L, H at a residue corresponding to position 223 in SEQ ID NO: 1; N at a residue corresponding to position 232 in SEQ ID NO: 1; W, Y at a residue corresponding to position 233 in SEQ ID NO: 1; H at a residue corresponding to position 234 in SEQ ID NO: 1; T at a residue corresponding to position 238 in SEQ ID NO: 1; L at a residue corresponding to position 258 in SEQ ID NO: 1; Y, H at a residue corresponding to position 288 in SEQ ID NO: 1; V, T, C, I, L at a residue corresponding to position 318 in SEQ ID NO: 1; W, Y at a residue corresponding to position 320 in SEQ ID NO: 1; C at a residue corresponding to position 321 in SEQ ID NO: 1; G at a residue corresponding to position 323 in SEQ ID NO: 1; S at a residue corresponding to position 364 in SEQ ID NO: 1; C, A at a residue corresponding to position 513 in SEQ ID NO: 1; or D at a residue corresponding to position 523 in SEQ ID NO: 1; or any combination thereof.

In some embodiments, provided herein is microbial organism (e.g., host microbial organism) that has a recombinant nucleic acid encoding an engineered ACL having one or more amino acid alterations comprising: I4M, N7D, N7K, N7Q, N7S, N7T, A81, A8S, T9A, T9K, T9S, I10F, I10H, I10P, I10V, I10W, I10Y, N12D, N12E, N12H, N12P, I13C, I13M, V15D, V15I, V15L, V15P, D16E, D16H, D16N, Y19F, Y19L, Y19P, Y19Q, D20W, K22S, G30D, G31A, E35G, E35P, T36H, S39A, V40C, V40F, V40L, V40M, A43G, K44D, K44H, K44S, K44T, Q47R, Q47RTYTQKEL (“RTYTQKEL” disclosed as SEQ ID NO: 15), Q47RTYTQREL (“RTYTQREL” disclosed as SEQ ID NO: 16), Q47RTYT (“RTYT” disclosed as SEQ ID NO: 17), d48, d49, L49Y, d50, S50T, d51, Q51F, Q51H, Q51L, Q51R, Q51W, Q51Y, d52, A52K, A52R, d53, d54, M54L, W55L, K60R, Y61F, Y61L, L64A, L64G, S66R, S67A, Y69L, C72G, C72T, R73F, R73G, R73L, R73V, H74K, D76T, E78V, D80G, P81PG, S82A, S82E, S82G, S82N, S82R, S82T, d83, M83K, M83P, M83R, M83S, V86I, L87I, L87V, L87VAV, d88, I88A, d89, T89I, T89L, T89V, F90C, F90L, F90M, F90V, F90W, F91A, F91L, F91M, F91S, V94C, V94CIEAA (“CIEAA” disclosed as SEQ IDNO: 18), V94S, V94T, V94TIEAA (“TIEAA” disclosed as SEQ ID NO: 19), d95, d96, L96E, d97, d98, P98A, P98L, V99C, V99M, V100S, V100M, V100L, H101F, H101M, W102H, W102F, W102L, A103G, A104V, A104L, A104T, L105V, L105W, L105H, L105A, L105I, D106S, D106Y, D106R, D106K, L107A, L107C, L107V, A109L, A109V, A109Q, I111V, I111A, I111M, I111L, I111C, I111VTPAN (“VTPAN” disclosed as SEQ ID NO: 20), S112T, d112, d113, P113L, d114, G114A, G114L, G114V, d115, S115N, T116A, T116W, T116P, G117S, S119T, Q121R, Q121D, Q126H, F127M, F127L, F127MRHTG (“MRHTG” disclosed as SEQ ID NO: 21), F127MRITG (“MRITG” disclosed as SEQ ID NO: 22), d128, d129, V129I, V129H, d130, d131, T131G, T131D, V136L, Y137T, A138D, A138S, K139E, A140E, A140K, A140D, V144T, T149R, K150I, K150L, Y152A, Y152T, Y152C, Y152I, N153G, N153S, A159G, E162S, E162H, E162R, E162SIDAF (“SIDAF” disclosed as SEQ ID NO: 23), E162RIESF (“RIESF” disclosed as SEQ ID NO: 24), E162HFDSF (“HFDSF” disclosed as SEQ ID NO: 25), E162HFDAF (“HFDAF” disclosed as SEQ ID NO: 26), E162HIDSF (“HIDSF” disclosed as SEQ ID NO: 27), L163F, L163I, d163, d164, E164D, d165, A165S, d166, L166F, D167L, A170G, M172L, M172A, M172D, R182Q, H189E, H189G, S191A, R194D, I195T, I195P, I195M, A196T, A196I, Y197W, Y197C, L198Y, L198V, L198S, L198W, L198I, L198M, L198T, G199M, G199L, G199W, G199P, G199F, G199C, G199A, M200W, S201F, S201T, S201N, S201P, S201Y, S201L, S202T, S202H, S202A, S202Q, S202E, S202L, S202G, S205T, V208F, S209P, A211G, V212A, V212C, R213M, R213V, R213E, L214V, T215S, S217Q, S217R, S217Y, S217H, S217K, S217G, T220W, T220M, T220V, S221T, S221A, C222S, C222N, S223A, S223N, S223I, S223L, S223H, E224T, E224Q, E224H, E224I, E224N, E224M, I225L, I225A, I225F, I225Q, S226Y, S226C, S226T, S226W, N227G, N227E, N227M, N227T, N227A, N227GKWSQD (“GKWSQD” disclosed as SEQ ID NO: 28), N227GKWSQSY (“GKWSQSY” disclosed as SEQ ID NO: 29), N227GKWSQ (“GKWSQ” disclosed as SEQ ID NO: 30), N227GKWSQDLK (“GKWSQDLK” disclosed as SEQ ID NO: 31), N227GKWSQQLQ (“GKWSQQLQ” disclosed as SEQ ID NO: 32), d228, d229, A229F, A229S, A229L, A229G, A229W, A229I, A229M, A229H, d230, S230R, S230M, S230P, S230T, S230E, S230Q, S230A, d231, E231Q, E231P, E231H, E231QDLQ (“QDLQ” disclosed as SEQ ID NO: 33), d232, S232W, S232A, S232Y, S232N, S232K, S232Q, S232D, d233, L233W, L233R, L233M, L233Y, L233P, d234, A234F, A234Y, A234T, A234K, A234R, A234Q, A234P, A234G, A234H, A234E, T235H, T235S, T235R, T235K, T235M, T235Q, T235D, T235G, Q238P, Q238N, Q238H, Q238T, Q238D, Q238DISAC (“DISAC” disclosed as SEQ ID NO: 34), Q238DVS, I239V, I239F, I239R, d239, d240, A240F, A240H, A240T, A240W, A240S, A240L, A240C, A240V, d241, A241C, A241M, A241P, A241I, d242, A242G, A242V, A242C, I243L, I243C, I243M, I244M, I244V, V246W, V246F, V246L, V246Y, S247F, S247C, H248S, H248P, L249A, L249V, L249M, F250M, F250A, F250W, G251A, G251H, L252I, L252M, L252W, L252Q, S253T, S253Q, S253N, S253I, S253H, S253V, S253M, K254E, K254I, K254T, K254Y, K254H, K254V, K254L, K254P, K254A, K254M, K254F, K254S, K254G, F255M, F255T, F255I, F255N, F255Y, F255C, F255ILLANV (“ILLANV” disclosed as SEQ ID NO: 35), F255ILLANVHY (“ILLANVHY” disclosed as SEQ ID NO: 36), F255ILLLNVHY (“ILLLNVHY” disclosed as SEQ ID NO: 37), F255ILLLNV (“ILLLNV” disclosed as SEQ ID NO: 38), F255ILLLNVHK (“ILLLNVHK” disclosed as SEQ ID NO: 39), F255ILLHNVHK (“ILLHNVHK” disclosed as SEQ ID NO: 40), F255ILLSNVHY (“ILLSNVHY” disclosed as SEQ ID NO: 41), L256I, L256V, L256H, L256C, L256F, d256, d257, I257L, I257T, I257N, d258, G258S, G258L, G258P, G258M, G258H, G258T, G258A, d259, N259C, N259A, d260, P260F, P260L, P260M, P260V, d261, H261Y, H261M, H261W, H261A, H261F, H261L, H261S, d262, A262M, A262K, A262Y, A262R, A262G, A262C, A262L, A262V, A262S, A262N, A264G, A264W, A264I, A264S, A264V, T265R, T265C, T265H, T265K, T266I, T266V, T266Q, T266C, V267Y, Y268V, Y268M, Y268T, Y268F, Y268IMPR (“IMPR” disclosed as SEQ ID NO: 42), Y268IMNR (“IMNR” disclosed as SEQ ID NO: 43), H269M, d269, d270, N270P, d271, G271R, G271Q, F272W, L274P, L274PEEMLRAIQ (“PEEMLRAIQ” disclosed as SEQ ID NO: 44), L274PEEMLQAIQ (“PEEMLQAIQ” disclosed as SEQ ID NO: 45), 1275D, I275A, 1275E, I275R, I275K, I275EEMLQAIQ (“EEMLQAIQ” disclosed as SEQ ID NO: 46), I275EEMLRAIQ (“EEMLRAIQ” disclosed as SEQ ID NO: 47), d275-282, I275KEMLQAIQ (“KEMLQAIQ” disclosed as SEQ ID NO: 48), d276, d277, V277M, V277Y, V277L, V277F, d278, d279, E279Q, E279R, E279QAIQ (“QAIQ” disclosed as SEQ ID NO: 49), E279RAIQ (“RAIQ” disclosed as SEQ ID NO: 50), d280, d281, Q281V, Q281M, Q281L, Q281I, d282, K282Q, V286P, V286C, N287T, N287TVFFA (“TVFFA” disclosed as SEQ ID NO: 51), N287TSFFA (“TSFFA” disclosed as SEQ ID NO: 52), N287TFFFA (“TFFFA” disclosed as SEQ ID NO: 53), N287TMFFA (“TMFFA” disclosed as SEQ ID NO: 54), N287TVFHA (“TVFHA” disclosed as SEQ ID NO: 55), S288H, S288F, S288M, S288Y, S288R, S288I, S288V, S288T, S288C, d288, d289, W289M, W289V, W289A, W289L, W289F, W289I, d290, T290A, T290W, T290H, T290C, T290F, T290V, T290P, T290L, d291, L291A, L291M, L291G, L291V, P294A, P294T, I295L, d295, I296M, I296L, V297M, V297Q, V297I, V297A, V297L, V297T, L298M, L298A, L298F, L298R, L298IM, L299M, L299F, T300A, I304M, I304R, 1304E, E306D, I309L, P310L, P310D, S311L, A314S, A314SHVRSV (“SHVRSV” disclosed as SEQ ID NO: 56), A314SHVRTV (“SHVRTV” disclosed as SEQ ID NO: 57), H315S, d315, d316, M316V, M316L, M316I, d317, R317H, R317N, R317Q, R317T, d318, A318Y, A318M, A318T, A318S, A318C, A318Q, A318R, A318H, A318V, A318W, A318I, A318L, d319, I319C, I319F, I319V, I319L, I319M, L320F, L320I, L320V, L320Y, L320T, L320M, L320W, S321C, A323G, A324S, A324M, L326V, L326I, L326C, G327S, A328H, A328V, A328R, A328S, A328RELE (“RELE” disclosed as SEQ ID NO: 58), N329E, d329, N329ELE, d330, V330L, V330T, V330I, d331, T331M, T331E, T331C, T331L, T331I, L334A, L334W, L334C, L334M, L334I, L335Q, L335R, T336K, V338M, T339S, N341H, N341T, K342L, K342F, E348D, E348H, E348Y, L351V, L351C, L351T, L351F, L351A, L351W, R352E, R352K, R352D, I353V, I353L, I353M, V354C, V354F, V354L, V354Y, V354A, V354K, V354S, V354I, Q355E, G356A, Y357W, L359M, T360S, T362C, T362S, S363A, S363T, S363G, P364C, P364S, V365L, V365I, V365M, V365C, A366S, A366V, A366T, T367A, T367C, T367S, T367H, L368H, F369H, F369Y, F369I, F369C, F369L, F369V, F369W, F369S, F369T, F369M, D370Y, D370R, D370T, D370S, D370H, D370G, D370N, D370Q, P371W, E372K, E372W, E372Q, E372H, E372S, E372R, E372N, D373S, D373H, D373P, D373Q, D373E, K374D, K374T, K374A, K374R, K374S, K374P, K374W, K374E, K374Q, K374H, E375P, R376T, R376K, R376E, R376Q, R376H, R376D, H377Y, H377R, H377N, I378S, 1378Y, 1378P, 1378V, 1378A, R379G, S380T, C381V, C381S, C381I, K383Q, K383H, K383T, K383Y, K383W, K383P, L384I, L384P, L384A, L384M, P386S, P386A, N387G, N387S, N387H, S388T, S388M, S388V, S388L, Q389R, Q389K, Q389E, V390C, V390L, V390A, R391K, N394D, V398D, V398N, A402V, A402L, A402I, D406L, D406V, P407D, L410N, L410Q, L410NTENAPR (“NTENAPR” disclosed as SEQ ID NO: 59), L410NTENAPD (“NTENAPD” disclosed as SEQ ID NO: 60), D411T, D411TENAPD (“TENAPD” disclosed as SEQ ID NO: 61), D411TQNAPR (“TQNAPR” disclosed as SEQ ID NO: 62), d411, D411TENAPR (“TENAPR” disclosed as SEQ ID NO: 63), D411TQNAPN (“TQNAPN” disclosed as SEQ ID NO: 64), d412, E412Q, d413, A413N, A413NNPN (“NNPN” disclosed as SEQ ID NO: 65), d414, I414A, I414S, I414N, d415, K415P, d416, Q416D, Q416R, Q416N, Q416E, T418P, L419G, P420E, L424M, L424V, W425V, W425L, I426M, I426L, Q430T, Q430N, Q430S, V431I, V431L, M432T, M432F, D433S, D433K, H436W, N442T, E443R, E443K, E443RASFA (“RASFA” disclosed as SEQ ID NO: 66), d444, d445, C445M, C445T, C445S, C445A, d446, d447, V447A, D455S, D455G, Y459G, Y459E, Y459D, R461D, R461G, H462R, H462G, F464L, F464Y, R465H, V469M, A470C, A470G, L471Y, L471K, L471M, L471W, L471I, L471R, V472I, V472F, V472C, V472Y, V472L, V472M, K474E, K474EDGH (“EDGH” disclosed as SEQ ID NO: 67), K474EDGY (“EDGY” disclosed as SEQ ID NO: 68), Q475D, d475, d476, d477, R477H, R477F, R477N, R477E, R477Y, Y478W, Y478I, Y478M, M479T, M479H, M479Y, M479F, M479W, I480V, I480L, V481C, V481I, V481T, T484L, E486D, K489I, S490R, S490C, S490V, Q491G, Q491R, Q491N, K493F, K493H, K493Y, K493L, Q494N, A496F, A496S, A496Y, M503L, M503V, M503I, G506R, G506RHPD (“RHPD” disclosed as SEQ ID NO: 69), d507, d508, A508P, d509, Q509D, T513A, T513V, T513C, I518V, Q519P, V521E, V521P, K523Q, K523A, K523R, K523M, K523Y, K523D, G524S, G524A, N525S, N525T, N525D, N525G, N525E, A527P, A527T, A527V, P528A, P528V, R529V, R529A, Y539L, Y539V, D540T, V542E, V542D, T546A, T546K, Q551K, Q551R, P553S, K554S, K554P, K554H, Y555F, Q557K, H559P, A560G, I562V, d562, V564F, I565V, I565F, I565FV, D566E, D566VE, A583Q, A583E, or any combination thereof. It is understood that when multiple alterations without any delimitating characters (e.g., commas and/or spaces) are used in reference to an alteration at a specific position, the alteration is conjunctive and all identified alterations alter the corresponding specific position. For example, a G506RHPD (“RHPD” disclosed as SEQ ID NO: 69) means that R replaces the G at position 506 and HPD are inserted adjacent to the R. It is also understood that when a lower case “d” is used in reference to an alteration at a specific position, the alteration is a deletion at the specific position. For example the alteration d475 means that the residue at position 475 has been deleted.

In some embodiments, provided herein is microbial organism (e.g., host microbial organism) that has a recombinant nucleic acid encoding an engineered ACL having one or more amino acid alterations comprising: N7K, N7S, A8S, I10W, I10Y, I10F, N12E, N12P, N12D, V15I, D16E, Y19F, A52R, A52K, M54L, L64A, S66R, S67A, R73L, H74K, S82T, L87I, F90L, F90M, V94T, V100L, I111V, S115N, G117S, S119T, Q121D, V129I, T131G, A138D, A138S, K139E, A140K, A140E, A140D, V144T, K150L, K150I, A165S, L166F, M172L, M172A, M172D, R182Q, S191A, G199A, S209P, V212C, S217R, S221T, S221A, S223N, S223I, S223A, S223L, S223H, E224Q, N227E, N227T, N227A, A229I, A229L, A229M, S230Q, S230A, E231H, E231Q, E231P, S232Y, S232K, S232Q, S232N, L233W, L233Y, A234Y, A234F, A234H, T235S, Q238D, Q238H, Q238T, A240T, A240H, A240C, A240S, I243C, I243M, L249M, L249V, S253H, S253M, S253Q, K254M, L256I, L256V, G258T, G258H, G258A, G258L, G258M, N259A, A262K, A262C, A262N, Y268F, G271R, G271Q, I275E, V277L, V277M, Q281V, S288V, S288I, S288F, S288T, S288H, S288Y, T290V, T290A, I296L, I296M, V297I, T300A, I304E, E306D, M316L, A318S, A318R, A318H, A318W, A318Y, A318M, A318T, A318C, A318Q, A318V, A318I, A318L, L320M, L320F, L320T, L320Y, L320W, S321C, A323G, A328V, A328H, A328R, V330I, V330L, T331I, T336K, R352K, I353V, I353L, S363G, P364S, V365C, V365L, V365I, V365M, A366V, F369H, F369S, F369T, F369M, E372S, E372K, E372H, E372N, K374Q, K374R, K374T, K374P, K374H, K374S, K374A, K374D, K374E, R376T, I378A, I378P, I378S, I378V, S388T, V390C, V390A, N394D, V398N, D406L, P407D, Q416N, Q416E, M432F, E443R, E443K, C445A, D455S, D455G, H462R, V469M, A470G, L471R, L471M, L471W, I480V, V481I, V481C, T484L, E486D, S490C, Q491R, Q491G, K493L, K493H, K493Y, Q494N, A496Y, A508P, T513A, T513V, T513C, I518V, Q519P, V521E, V521P, K523R, K523Q, K523A, K523Y, K523M, K523D, N525D, N525S, N525T, N525G, N525E, T546K, K554H, K554S, K554P, Y555F, A560G, I565V, A583Q, or A583E.

In some embodiments, provided herein is microbial organism (e.g., host microbial organism) that has a recombinant nucleic acid encoding an engineered ACL having one or more amino acid alterations comprising: I10F, G199A, S223L, S223H, S223A, A229M, S230Q, S230A, E231P, S232N, L233W, L233Y, A234H, T235S, Q238T, I243C, I243M, L256I, L256V, G258L, G258M, V277M, S288Y, S288H, T300A, A318V, A318T, A318C, A318I, A318L, A318Q, A318M, A318Y, L320W, L320Y, S321C, A323G, A328R, V330L, P364S, V365I, V365M, F369M, E372N, K374E, I378V, H462R, V469M, I480V, V481C, T484L, T513C, T513A, T513V, K523D, N525G, N525E, N525T, K554S, K554P, A560G, or A583E.

In some embodiments, provided herein is microbial organism (e.g., host microbial organism) that has a recombinant nucleic acid encoding an engineered ACL having one or more amino acid alterations comprising: L233W, P364S, S232N, L320W, A318V, S321C, S223L, L320Y, T513C, Q238T, L233Y, K523D, A318T, S288Y, A318C, A234H, A323G, T513A, S288H, S223H, A318I, A318L, G258L.

In some embodiments, provided herein is microbial organism (e.g., host microbial organism) that has a recombinant nucleic acid encoding an engineered ACL provided herein has an amino acid sequence that is a variant of SEQ ID NO: 1 that includes one or more alterations as described in TABLE 8, wherein the portion, other than the one or more alterations described in TABLE 8, of the engineered ACL has at least 65%, 70%, 75%, 80%, 85%, 90%, 95%, 98% or 99% sequence identity, or is identical, to an amino acid sequence referenced as SEQ ID NO: 1. Accordingly, in some embodiments, provided herein is microbial organism (e.g., host microbial organism) that has a recombinant nucleic acid encoding an engineered ACL that has an amino acid sequence that includes one or more alterations as described in TABLE 8 and the portion, other than the alteration described in TABLE 8, of the engineered ACL has at least 65% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and the portion, other than the alteration described in TABLE 8, of the engineered ACL has at least 70% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and the portion, other than the alteration described in TABLE 8, of the engineered ACL has at least 75% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and the portion, other than the alteration described in TABLE 8, of the engineered ACL has at least 80% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and the portion, other than the alteration described in TABLE 8, of the engineered ACL has at least 85% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and the portion, other than the alteration described in TABLE 8, of the engineered ACL has at least 90% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and the portion, other than the alteration described in TABLE 8, of the engineered ACL has at least 95% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and the portion, other than the alteration described in TABLE 8, of the engineered ACL has at least 98% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and the portion, other than the alteration described in TABLE 8, of the engineered ACL has at least 99% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and the portion, other than the alteration described in TABLE 8, of the engineered ACL is identical to SEQ ID NO: 1.

In some embodiments, provided herein is microbial organism (e.g., host microbial organism) that has a recombinant nucleic acid encoding an engineered ACL that is a variant of a reference polypeptide, wherein the reference polypeptide has an amino acid sequence of SEQ ID NO: 2, and the engineered ACL has one or more alterations at a position described in TABLE 9 relative to SEQ ID NO: 2. In some embodiments, the microbial organism has a recombinant nucleic acid that encodes an engineered ACL that includes one or more amino acid alterations at a residue corresponding to position 5, 6, 7, 8, 10, 12, 13, 14, 15, 16, 19, 20, 22, 25, 28, 37, 39, 40, 41, 43, 44, 45, 48, 50, 51, 53, 57, 58, 59, 61, 62, 63, 66, 67, 69, 71, 72, 74, 75, 76, 77, 78, 80, 81, 83, 84, 85, 86, 87, 88, 92, 93, 94, 95, 97, 98, 99, 100, 101, 103, 104, 106, 107, 108, 109, 110, 113, 114, 115, 116, 119, 120, 121, 122, 123, 124, 125, 126, 127, 128, 130, 131, 136, 137, 140, 141, 142, 145, 147, 148, 149, 150, 151, 157, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 216, 218, 219, 220, 221, 222, 224, 225, 226, 227, 228, 230, 231, 232, 233, 234, 235, 236, 237, 238, 239, 240, 241, 242, 243, 244, 245, 246, 247, 251, 252, 253, 254, 255, 256, 257, 259, 260, 262, 263, 264, 265, 266, 267, 268, 269, 270, 271, 272, 273, 274, 275, 277, 278, 279, 281, 282, 283, 284, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 341, 342, 343, 344, 345, 346, 347, 348, 349, 351, 352, 353, 354, 355, 357, 358, 359, 361, 362, 363, 365, 366, 367, 368, 369, 370, 372, 373, 375, 376, 377, 378, 379, 380, 381, 382, 383, 384, 385, 386, 387, 388, 389, 391, 392, 394, 395, 396, 398, 399, 400, 401, 402, 404, 405, 406, 407, 409, 411, 413, 414, 415, 416, 417, 418, 419, 420, 421, 425, 430, 432, 433, 434, 435, 441, 442, 443, 446, 447, 449, 450, 451, 452, 453, 455, 456, 458, 459, 460, 461, 462, 463, 464, 465, 466, 467, 468, 469, 470, 471, 473, 474, 476, 478, 479, 480, 481, 483, 485, 490, 492, 493, 496, 497, 498, 502, 504, 507, 508, 510, 511, 512, 513, 514, 515, 516, 518, 519, 521, 526, 527, 532, 535, 540, 542, 546, 552, 554, 558, 560, 561, 563, 564, 565, 566, 567, 568, 569, 570, 572, 573, 574, 575, 577, 578, 583, 584, 585, 587, 588, 591, 593, 595, 596, 598, 599, 601, more than one of the foregoing, or a combination thereof, in SEQ ID NO: 2. In some embodiments, the microbial organism has a recombinant nucleic acid that encodes an engineered ACL that includes one or more amino acid alterations at a residue corresponding to position 5, 6, 7, 8, 10, 12, 13, 14, 15, 16, 19, 20, 22, 25, 28, 37, 39, 40, 41, 43, 45, 50, 51, 53, 57, 58, 59, 61, 62, 63, 67, 69, 71, 72, 74, 75, 76, 77, 78, 80, 81, 83, 84, 85, 86, 87, 88, 92, 93, 94, 95, 97, 98, 99, 100, 101, 103, 104, 106, 108, 109, 110, 113, 116, 119, 120, 121, 123, 124, 125, 126, 127, 128, 130, 131, 136, 137, 140, 141, 142, 145, 147, 148, 149, 150, 151, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 218, 219, 220, 221, 222, 224, 226, 227, 228, 230, 232, 233, 234, 235, 236, 237, 238, 239, 240, 241, 242, 243, 244, 245, 246, 247, 252, 253, 254, 255, 256, 260, 262, 263, 264, 265, 266, 267, 268, 270, 271, 272, 273, 274, 275, 277, 278, 279, 281, 283, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 342, 343, 344, 345, 346, 347, 348, 349, 351, 352, 353, 354, 355, 357, 358, 359, 362, 363, 365, 366, 367, 368, 369, 370, 375, 376, 377, 378, 379, 380, 381, 382, 383, 384, 385, 386, 387, 388, 389, 391, 392, 394, 396, 398, 399, 400, 401, 404, 406, 407, 409, 411, 414, 416, 419, 420, 421, 425, 430, 432, 433, 434, 435, 441, 442, 443, 446, 447, 450, 451, 452, 453, 456, 460, 461, 462, 463, 465, 466, 467, 468, 469, 470, 471, 473, 476, 478, 479, 480, 481, 485, 490, 492, 498, 507, 508, 510, 511, 512, 514, 515, 516, 518, 519, 521, 526, 527, 532, 535, 540, 542, 546, 552, 554, 560, 561, 563, 564, 565, 566, 567, 568, 569, 570, 572, 573, 574, 575, 577, 578, 583, 584, 585, 587, 588, 591, 593, 595, 596, 598, 599, 601, more than one of the foregoing, or a combination thereof, in SEQ ID NO: 2. In some embodiments, the microbial organism has a recombinant nucleic acid that encodes an engineered ACL that includes one or more amino acid alterations at a residue corresponding to position 5, 6, 7, 8, 10, 12, 14, 15, 16, 19, 20, 22, 28, 39, 41, 43, 45, 50, 57, 58, 61, 62, 63, 67, 69, 71, 72, 76, 77, 78, 81, 84, 85, 86, 87, 92, 93, 94, 95, 98, 104, 108, 109, 113, 116, 119, 120, 123, 124, 125, 126, 127, 130, 131, 136, 140, 141, 142, 145, 147, 148, 149, 151, 159, 161, 163, 164, 167, 168, 169, 170, 173, 175, 176, 177, 181, 183, 185, 186, 187, 190, 191, 193, 194, 195, 201, 205, 209, 214, 218, 219, 220, 221, 226, 228, 230, 232, 234, 237, 238, 239, 241, 242, 243, 244, 246, 247, 253, 254, 260, 262, 263, 266, 267, 272, 273, 274, 278, 279, 281, 283, 285, 290, 291, 292, 295, 296, 298, 300, 302, 303, 304, 305, 307, 311, 313, 314, 315, 316, 318, 321, 322, 330, 333, 334, 336, 337, 338, 339, 340, 344, 345, 346, 347, 348, 349, 351, 352, 353, 354, 355, 357, 358, 359, 362, 363, 365, 367, 369, 375, 376, 379, 382, 383, 384, 386, 387, 388, 389, 399, 400, 401, 404, 407, 409, 416, 419, 420, 421, 430, 432, 433, 434, 441, 442, 446, 450, 452, 453, 456, 460, 461, 465, 466, 471, 473, 476, 479, 480, 481, 485, 490, 492, 507, 511, 514, 515, 516, 518, 519, 521, 526, 527, 532, 540, 542, 546, 554, 561, 564, 565, 566, 567, 568, 570, 572, 574, 575, 577, 584, 587, 595, 596, 599 or a combination thereof, in SEQ ID NO: 2. In some embodiments, the microbial organism has a recombinant nucleic acid that encodes an engineered ACL that includes one or more amino acid alterations at a residue corresponding to position 5, 6, 7, 8, 10, 12, 14, 15, 19, 20, 22, 41, 45, 50, 58, 62, 63, 67, 69, 72, 76, 77, 81, 86, 87, 92, 93, 94, 95, 98, 104, 108, 113, 116, 120, 123, 124, 125, 126, 127, 130, 136, 140, 141, 142, 145, 147, 148, 149, 151, 159, 161, 163, 164, 167, 168, 169, 175, 176, 177, 185, 186, 187, 190, 191, 193, 195, 201, 205, 209, 214, 219, 220, 221, 228, 230, 232, 234, 237, 238, 239, 241, 242, 243, 244, 246, 247, 253, 254, 260, 263, 266, 267, 272, 273, 274, 278, 279, 281, 283, 291, 292, 295, 298, 300, 302, 303, 304, 305, 307, 311, 313, 314, 315, 318, 321, 322, 330, 333, 334, 336, 337, 338, 340, 344, 348, 349, 351, 353, 354, 355, 357, 358, 359, 362, 363, 365, 367, 369, 376, 379, 382, 383, 386, 387, 388, 389, 399, 400, 401, 404, 407, 409, 420, 421, 430, 432, 433, 434, 442, 446, 450, 452, 453, 456, 460, 461, 465, 466, 473, 476, 479, 480, 481, 485, 492, 507, 514, 515, 516, 518, 519, 521, 526, 527, 532, 540, 542, 554, 564, 565, 567, 568, 570, 572, 575, 584, 587, 595, 596 or a combination thereof, in SEQ ID NO: 2.

In some embodiments, provided herein is microbial organism (e.g., host microbial organism) that has a recombinant nucleic acid encoding an engineered ACL having one or more alterations described in TABLE 9. Accordingly, in some embodiments, the microbial organism has a recombinant nucleic acid that encodes an engineered ACL having: R, E at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; K, D, P, N, R at a residue corresponding to position 10 in SEQ ID NO: 2; A, I, V, M at a residue corresponding to position 12 in SEQ ID NO: 2; C at a residue corresponding to position 13 in SEQ ID NO: 2; P, A, Q at a residue corresponding to position 14 in SEQ ID NO: 2; E, L, S, R at a residue corresponding to position 15 in SEQ ID NO: 2; C, L, I at a residue corresponding to position 16 in SEQ ID NO: 2; D, R at a residue corresponding to position 19 in SEQ ID NO: 2; H, K, R, D at a residue corresponding to position 20 in SEQ ID NO: 2; D, E at a residue corresponding to position 22 in SEQ ID NO: 2; N at a residue corresponding to position 25 in SEQ ID NO: 2; M at a residue corresponding to position 28 in SEQ ID NO: 2; C at a residue corresponding to position 37 in SEQ ID NO: 2; R at a residue corresponding to position 39 in SEQ ID NO: 2; T at a residue corresponding to position 40 in SEQ ID NO: 2; M, L, V, I at a residue corresponding to position 41 in SEQ ID NO: 2; S at a residue corresponding to position 43 in SEQ ID NO: 2; M at a residue corresponding to position 44 in SEQ ID NO: 2; I, V at a residue corresponding to position 45 in SEQ ID NO: 2; P at a residue corresponding to position 48 in SEQ ID NO: 2; I, F, R, V, L at a residue corresponding to position 50 in SEQ ID NO: 2; R at a residue corresponding to position 51 in SEQ ID NO: 2; L at a residue corresponding to position 53 in SEQ ID NO: 2; F at a residue corresponding to position 57 in SEQ ID NO: 2; V, L, M, F at a residue corresponding to position 58 in SEQ ID NO: 2; V at a residue corresponding to position 59 in SEQ ID NO: 2; P, E at a residue corresponding to position 61 in SEQ ID NO: 2; D at a residue corresponding to position 62 in SEQ ID NO: 2; D at a residue corresponding to position 63 in SEQ ID NO: 2; G at a residue corresponding to position 66 in SEQ ID NO: 2; K at a residue corresponding to position 67 in SEQ ID NO: 2; F, Y, L at a residue corresponding to position 69 in SEQ ID NO: 2; F, W at a residue corresponding to position 71 in SEQ ID NO: 2; A, R at a residue corresponding to position 72 in SEQ ID NO: 2; F at a residue corresponding to position 74 in SEQ ID NO: 2; D at a residue corresponding to position 75 in SEQ ID NO: 2; T, D, A, R at a residue corresponding to position 76 in SEQ ID NO: 2; R at a residue corresponding to position 77 in SEQ ID NO: 2; A at a residue corresponding to position 78 in SEQ ID NO: 2; K at a residue corresponding to position 80 in SEQ ID NO: 2; C at a residue corresponding to position 81 in SEQ ID NO: 2; S, H, Q, N at a residue corresponding to position 83 in SEQ ID NO: 2; Y, H at a residue corresponding to position 84 in SEQ ID NO: 2; F at a residue corresponding to position 85 in SEQ ID NO: 2; A, Q at a residue corresponding to position 86 in SEQ ID NO: 2; R at a residue corresponding to position 87 in SEQ ID NO: 2; L at a residue corresponding to position 88 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; A at a residue corresponding to position 97 in SEQ ID NO: 2; M at a residue corresponding to position 98 in SEQ ID NO: 2; L at a residue corresponding to position 99 in SEQ ID NO: 2; C at a residue corresponding to position 100 in SEQ ID NO: 2; P at a residue corresponding to position 101 in SEQ ID NO: 2; R at a residue corresponding to position 103 in SEQ ID NO: 2; L, W, A at a residue corresponding to position 104 in SEQ ID NO: 2; W, F at a residue corresponding to position 106 in SEQ ID NO: 2; V, P, I, L, W, F at a residue corresponding to position 107 in SEQ ID NO: 2; V at a residue corresponding to position 108 in SEQ ID NO: 2; V at a residue corresponding to position 109 in SEQ ID NO: 2; M, F, Y, A, D at a residue corresponding to position 110 in SEQ ID NO: 2; T, C, V at a residue corresponding to position 113 in SEQ ID NO: 2; M, W, L, G, A at a residue corresponding to position 114 in SEQ ID NO: 2; A at a residue corresponding to position 115 in SEQ ID NO: 2; I at a residue corresponding to position 116 in SEQ ID NO: 2; V, T, I at a residue corresponding to position 119 in SEQ ID NO: 2; M, F, V, A, L, W, Y at a residue corresponding to position 120 in SEQ ID NO: 2; T at a residue corresponding to position 121 in SEQ ID NO: 2; A at a residue corresponding to position 122 in SEQ ID NO: 2; M, V, I at a residue corresponding to position 123 in SEQ ID NO: 2; Y, S, N at a residue corresponding to position 124 in SEQ ID NO: 2; P, T, W at a residue corresponding to position 125 in SEQ ID NO: 2; M, N, R, A at a residue corresponding to position 126 in SEQ ID NO: 2; F, W, L at a residue corresponding to position 127 in SEQ ID NO: 2; R at a residue corresponding to position 128 in SEQ ID NO: 2; M at a residue corresponding to position 130 in SEQ ID NO: 2; E at a residue corresponding to position 131 in SEQ ID NO: 2; E, Q, K at a residue corresponding to position 136 in SEQ ID NO: 2; M at a residue corresponding to position 137 in SEQ ID NO: 2; M, C, I at a residue corresponding to position 140 in SEQ ID NO: 2; M at a residue corresponding to position 141 in SEQ ID NO: 2; N, Q at a residue corresponding to position 142 in SEQ ID NO: 2; G, R, D at a residue corresponding to position 145 in SEQ ID NO: 2; S at a residue corresponding to position 147 in SEQ ID NO: 2; L, V, A at a residue corresponding to position 148 in SEQ ID NO: 2; V, L at a residue corresponding to position 149 in SEQ ID NO: 2; V at a residue corresponding to position 150 in SEQ ID NO: 2; C at a residue corresponding to position 151 in SEQ ID NO: 2; L at a residue corresponding to position 157 in SEQ ID NO: 2; R, E at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 165 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, V, L at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 189 in SEQ ID NO: 2; V at a residue corresponding to position 190 in SEQ ID NO: 2; A, E at a residue corresponding to position 191 in SEQ ID NO: 2; T at a residue corresponding to position 192 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; P at a residue corresponding to position 196 in SEQ ID NO: 2; A, P, L at a residue corresponding to position 201 in SEQ ID NO: 2; T at a residue corresponding to position 205 in SEQ ID NO: 2; D at a residue corresponding to position 208 in SEQ ID NO: 2; I, T, P, V at a residue corresponding to position 209 in SEQ ID NO: 2; L, T, C, M, V, A at a residue corresponding to position 211 in SEQ ID NO: 2; L, M at a residue corresponding to position 212 in SEQ ID NO: 2; I, C, V, Q, M at a residue corresponding to position 213 in SEQ ID NO: 2; Y at a residue corresponding to position 214 in SEQ ID NO: 2; S at a residue corresponding to position 215 in SEQ ID NO: 2; G at a residue corresponding to position 216 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; T, M, L, R, K, H at a residue corresponding to position 221 in SEQ ID NO: 2; S at a residue corresponding to position 222 in SEQ ID NO: 2; A at a residue corresponding to position 224 in SEQ ID NO: 2; A at a residue corresponding to position 225 in SEQ ID NO: 2; M, I, V at a residue corresponding to position 226 in SEQ ID NO: 2; L, D, H, I at a residue corresponding to position 227 in SEQ ID NO: 2; S at a residue corresponding to position 228 in SEQ ID NO: 2; H, K at a residue corresponding to position 230 in SEQ ID NO: 2; N at a residue corresponding to position 231 in SEQ ID NO: 2; L, I, V at a residue corresponding to position 232 in SEQ ID NO: 2; L, V, M, W, A, I at a residue corresponding to position 233 in SEQ ID NO: 2; S, W at a residue corresponding to position 234 in SEQ ID NO: 2; Q, S at a residue corresponding to position 235 in SEQ ID NO: 2; A, C, I, M, V at a residue corresponding to position 236 in SEQ ID NO: 2; A, M, Q, R, E at a residue corresponding to position 237 in SEQ ID NO: 2; M, S, W, N, H at a residue corresponding to position 238 in SEQ ID NO: 2; F, M, V, C, L at a residue corresponding to position 239 in SEQ ID NO: 2; A, R at a residue corresponding to position 240 in SEQ ID NO: 2; A, H, E, Q at a residue corresponding to position 241 in SEQ ID NO: 2; V, C, T, W at a residue corresponding to position 242 in SEQ ID NO: 2; I, F, M, W, V at a residue corresponding to position 243 in SEQ ID NO: 2; E, P, Q, H, G at a residue corresponding to position 244 in SEQ ID NO: 2; M, L, H, W, Y at a residue corresponding to position 245 in SEQ ID NO: 2; I, T, M at a residue corresponding to position 246 in SEQ ID NO: 2; R, P, E at a residue corresponding to position 247 in SEQ ID NO: 2; E at a residue corresponding to position 251 in SEQ ID NO: 2; H, V at a residue corresponding to position 252 in SEQ ID NO: 2; Y, M, F, L, V at a residue corresponding to position 253 in SEQ ID NO: 2; I, F, V, M at a residue corresponding to position 254 in SEQ ID NO: 2; A, T, N, L, I, G, H, C, V, S at a residue corresponding to position 255 in SEQ ID NO: 2; F, V, I, C, Y at a residue corresponding to position 256 in SEQ ID NO: 2; M at a residue corresponding to position 257 in SEQ ID NO: 2; L, F at a residue corresponding to position 259 in SEQ ID NO: 2; H, S, Y, F at a residue corresponding to position 260 in SEQ ID NO: 2; C, T, A, I, M at a residue corresponding to position 262 in SEQ ID NO: 2; A, Y, H at a residue corresponding to position 263 in SEQ ID NO: 2; A at a residue corresponding to position 264 in SEQ ID NO: 2; M, L, W, F, C at a residue corresponding to position 265 in SEQ ID NO: 2; N, T, M, V at a residue corresponding to position 266 in SEQ ID NO: 2; I, G, M, W, F, L, V, C, A at a residue corresponding to position 267 in SEQ ID NO: 2; M, A, C, G, V at a residue corresponding to position 268 in SEQ ID NO: 2; L, M, I, V at a residue corresponding to position 269 in SEQ ID NO: 2; M, H, Y, T at a residue corresponding to position 270 in SEQ ID NO: 2; A, P, C, T, L at a residue corresponding to position 271 in SEQ ID NO: 2; C, V, P, A, T at a residue corresponding to position 272 in SEQ ID NO: 2; M, V, F, I at a residue corresponding to position 273 in SEQ ID NO: 2; Y, R, V, M, C, H, W at a residue corresponding to position 274 in SEQ ID NO: 2; G, C, S, H at a residue corresponding to position 275 in SEQ ID NO: 2; A, C at a residue corresponding to position 277 in SEQ ID NO: 2; C, T, A, H at a residue corresponding to position 278 in SEQ ID NO: 2; V, M, I at a residue corresponding to position 279 in SEQ ID NO: 2; F, L, V, M at a residue corresponding to position 281 in SEQ ID NO: 2; P, M, E, A, H at a residue corresponding to position 282 in SEQ ID NO: 2; E, D, P, R at a residue corresponding to position 283 in SEQ ID NO: 2; F, R, W at a residue corresponding to position 284 in SEQ ID NO: 2; R, F, W, D at a residue corresponding to position 285 in SEQ ID NO: 2; D, F at a residue corresponding to position 286 in SEQ ID NO: 2; P, A, V at a residue corresponding to position 287 in SEQ ID NO: 2; A at a residue corresponding to position 290 in SEQ ID NO: 2; M, V, L, P, I at a residue corresponding to position 291 in SEQ ID NO: 2; H at a residue corresponding to position 292 in SEQ ID NO: 2; I, L, A at a residue corresponding to position 294 in SEQ ID NO: 2; W, M, V, I at a residue corresponding to position 295 in SEQ ID NO: 2; D, Q at a residue corresponding to position 296 in SEQ ID NO: 2; M, V, L at a residue corresponding to position 298 in SEQ ID NO: 2; A at a residue corresponding to position 299 in SEQ ID NO: 2; Q at a residue corresponding to position 300 in SEQ ID NO: 2; Q at a residue corresponding to position 302 in SEQ ID NO: 2; V, I, A, P, C at a residue corresponding to position 303 in SEQ ID NO: 2; S at a residue corresponding to position 304 in SEQ ID NO: 2; W, Y, V, I, F, H, M at a residue corresponding to position 305 in SEQ ID NO: 2; V, Y, I, M, L, F at a residue corresponding to position 306 in SEQ ID NO: 2; P, W, Y, H, M at a residue corresponding to position 307 in SEQ ID NO: 2; T, A, S, L, G, V, M at a residue corresponding to position 308 in SEQ ID NO: 2; A, R, P, T at a residue corresponding to position 311 in SEQ ID NO: 2; I, M, V, F at a residue corresponding to position 312 in SEQ ID NO: 2; L, F, W at a residue corresponding to position 313 in SEQ ID NO: 2; Q, E, H at a residue corresponding to position 314 in SEQ ID NO: 2; R, A, K, L at a residue corresponding to position 315 in SEQ ID NO: 2; W, M, I, F at a residue corresponding to position 316 in SEQ ID NO: 2; H, D at a residue corresponding to position 318 in SEQ ID NO: 2; R, Q at a residue corresponding to position 321 in SEQ ID NO: 2; Q, K, A, H at a residue corresponding to position 322 in SEQ ID NO: 2; L, F at a residue corresponding to position 330 in SEQ ID NO: 2; G at a residue corresponding to position 331 in SEQ ID NO: 2; S at a residue corresponding to position 333 in SEQ ID NO: 2; I, M, V at a residue corresponding to position 334 in SEQ ID NO: 2; R, K at a residue corresponding to position 335 in SEQ ID NO: 2; T, I, M, Y, V, F, W at a residue corresponding to position 336 in SEQ ID NO: 2; L, M, F, A, V at a residue corresponding to position 337 in SEQ ID NO: 2; W, L, M, V at a residue corresponding to position 338 in SEQ ID NO: 2; F, T, S, C at a residue corresponding to position 339 in SEQ ID NO: 2; G at a residue corresponding to position 340 in SEQ ID NO: 2; A, S at a residue corresponding to position 341 in SEQ ID NO: 2; S at a residue corresponding to position 342 in SEQ ID NO: 2; P, A at a residue corresponding to position 343 in SEQ ID NO: 2; M, I, V, C at a residue corresponding to position 344 in SEQ ID NO: 2; S at a residue corresponding to position 345 in SEQ ID NO: 2; V, A, P, E at a residue corresponding to position 346 in SEQ ID NO: 2; E at a residue corresponding to position 347 in SEQ ID NO: 2; T, I, L at a residue corresponding to position 348 in SEQ ID NO: 2; M, F, L, A, I, Y at a residue corresponding to position 349 in SEQ ID NO: 2; F, R, Q, W at a residue corresponding to position 351 in SEQ ID NO: 2; I, M, Y at a residue corresponding to position 352 in SEQ ID NO: 2; R at a residue corresponding to position 353 in SEQ ID NO: 2; K at a residue corresponding to position 354 in SEQ ID NO: 2; A, R at a residue corresponding to position 355 in SEQ ID NO: 2; W, G, V, L, P, I, F, A at a residue corresponding to position 357 in SEQ ID NO: 2; Q at a residue corresponding to position 358 in SEQ ID NO: 2; H, Q at a residue corresponding to position 359 in SEQ ID NO: 2; F at a residue corresponding to position 361 in SEQ ID NO: 2; H, L, C at a residue corresponding to position 362 in SEQ ID NO: 2; D, V, I, G at a residue corresponding to position 363 in SEQ ID NO: 2; M, V, I at a residue corresponding to position 365 in SEQ ID NO: 2; I, V, L, F at a residue corresponding to position 366 in SEQ ID NO: 2; T, C, M, I, V, L at a residue corresponding to position 367 in SEQ ID NO: 2; N, Q, E, T at a residue corresponding to position 368 in SEQ ID NO: 2; G, A at a residue corresponding to position 369 in SEQ ID NO: 2; Y, F at a residue corresponding to position 370 in SEQ ID NO: 2; L, Q at a residue corresponding to position 372 in SEQ ID NO: 2; S at a residue corresponding to position 373 in SEQ ID NO: 2; A, C at a residue corresponding to position 375 in SEQ ID NO: 2; T, S, C, G at a residue corresponding to position 376 in SEQ ID NO: 2; G, M at a residue corresponding to position 377 in SEQ ID NO: 2; C, T, V at a residue corresponding to position 378 in SEQ ID NO: 2; C, V, I, L, A at a residue corresponding to position 379 in SEQ ID NO: 2; F, C, H, S, M, T, V at a residue corresponding to position 380 in SEQ ID NO: 2; N, F, L, C, V at a residue corresponding to position 381 in SEQ ID NO: 2; L, N, C, T, P at a residue corresponding to position 382 in SEQ ID NO: 2; D, N, R, P, Y at a residue corresponding to position 383 in SEQ ID NO: 2; P, N, D, W at a residue corresponding to position 384 in SEQ ID NO: 2; G, D at a residue corresponding to position 385 in SEQ ID NO: 2; D, V, L, T at a residue corresponding to position 386 in SEQ ID NO: 2; D, R, N, H, E at a residue corresponding to position 387 in SEQ ID NO: 2; K, H at a residue corresponding to position 388 in SEQ ID NO: 2; A, P, L at a residue corresponding to position 389 in SEQ ID NO: 2; S, T at a residue corresponding to position 391 in SEQ ID NO: 2; V, C at a residue corresponding to position 392 in SEQ ID NO: 2; R, K, I, Q, L at a residue corresponding to position 394 in SEQ ID NO: 2; A at a residue corresponding to position 395 in SEQ ID NO: 2; F, I, M, V at a residue corresponding to position 396 in SEQ ID NO: 2; D, H, N at a residue corresponding to position 398 in SEQ ID NO: 2; M, V, T, C, I at a residue corresponding to position 399 in SEQ ID NO: 2; D, Q at a residue corresponding to position 400 in SEQ ID NO: 2; M, I, C, L, A at a residue corresponding to position 401 in SEQ ID NO: 2; R at a residue corresponding to position 402 in SEQ ID NO: 2; V at a residue corresponding to position 404 in SEQ ID NO: 2; D at a residue corresponding to position 405 in SEQ ID NO: 2; E at a residue corresponding to position 406 in SEQ ID NO: 2; P at a residue corresponding to position 407 in SEQ ID NO: 2; Q, E, G at a residue corresponding to position 409 in SEQ ID NO: 2; D, S, V, G, N at a residue corresponding to position 411 in SEQ ID NO: 2; I, V at a residue corresponding to position 413 in SEQ ID NO: 2; W, L, V, C, Q at a residue corresponding to position 414 in SEQ ID NO: 2; I at a residue corresponding to position 415 in SEQ ID NO: 2; R at a residue corresponding to position 416 in SEQ ID NO: 2; S at a residue corresponding to position 417 in SEQ ID NO: 2; D at a residue corresponding to position 418 in SEQ ID NO: 2; H, S, Q, M at a residue corresponding to position 419 in SEQ ID NO: 2; V at a residue corresponding to position 420 in SEQ ID NO: 2; T, F at a residue corresponding to position 421 in SEQ ID NO: 2; H, L at a residue corresponding to position 425 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; T, V at a residue corresponding to position 434 in SEQ ID NO: 2; I, M, L at a residue corresponding to position 435 in SEQ ID NO: 2; F, M, Y at a residue corresponding to position 441 in SEQ ID NO: 2; K, H, Y, R, C at a residue corresponding to position 442 in SEQ ID NO: 2; S at a residue corresponding to position 443 in SEQ ID NO: 2; M, I, L, V at a residue corresponding to position 446 in SEQ ID NO: 2; A, G at a residue corresponding to position 447 in SEQ ID NO: 2; I at a residue corresponding to position 449 in SEQ ID NO: 2; D, M at a residue corresponding to position 450 in SEQ ID NO: 2; E at a residue corresponding to position 451 in SEQ ID NO: 2; P at a residue corresponding to position 452 in SEQ ID NO: 2; G at a residue corresponding to position 453 in SEQ ID NO: 2; E, G, R at a residue corresponding to position 455 in SEQ ID NO: 2; D, E, Q, N at a residue corresponding to position 456 in SEQ ID NO: 2; N, F, Y, W at a residue corresponding to position 458 in SEQ ID NO: 2; Y at a residue corresponding to position 459 in SEQ ID NO: 2; Y, F, I, R, W at a residue corresponding to position 460 in SEQ ID NO: 2; I, L, Y at a residue corresponding to position 461 in SEQ ID NO: 2; I, T, V at a residue corresponding to position 462 in SEQ ID NO: 2; D at a residue corresponding to position 463 in SEQ ID NO: 2; D at a residue corresponding to position 464 in SEQ ID NO: 2; L at a residue corresponding to position 465 in SEQ ID NO: 2; D, K, A at a residue corresponding to position 466 in SEQ ID NO: 2; K at a residue corresponding to position 467 in SEQ ID NO: 2; M at a residue corresponding to position 468 in SEQ ID NO: 2; L at a residue corresponding to position 469 in SEQ ID NO: 2; V at a residue corresponding to position 470 in SEQ ID NO: 2; T, V at a residue corresponding to position 471 in SEQ ID NO: 2; S, N at a residue corresponding to position 473 in SEQ ID NO: 2; E at a residue corresponding to position 474 in SEQ ID NO: 2; N, Y, L, F, V, I at a residue corresponding to position 476 in SEQ ID NO: 2; T, L at a residue corresponding to position 478 in SEQ ID NO: 2; P, V at a residue corresponding to position 479 in SEQ ID NO: 2; L, I, S, P, A, V at a residue corresponding to position 480 in SEQ ID NO: 2; P, E at a residue corresponding to position 481 in SEQ ID NO: 2; E at a residue corresponding to position 483 in SEQ ID NO: 2; A, L, E, R at a residue corresponding to position 485 in SEQ ID NO: 2; C at a residue corresponding to position 490 in SEQ ID NO: 2; P at a residue corresponding to position 492 in SEQ ID NO: 2; F at a residue corresponding to position 493 in SEQ ID NO: 2; H at a residue corresponding to position 496 in SEQ ID NO: 2; A at a residue corresponding to position 497 in SEQ ID NO: 2; C at a residue corresponding to position 498 in SEQ ID NO: 2; P at a residue corresponding to position 502 in SEQ ID NO: 2; H at a residue corresponding to position 504 in SEQ ID NO: 2; Y, V, W at a residue corresponding to position 507 in SEQ ID NO: 2; V at a residue corresponding to position 508 in SEQ ID NO: 2; A at a residue corresponding to position 510 in SEQ ID NO: 2; V, L at a residue corresponding to position 511 in SEQ ID NO: 2; I at a residue corresponding to position 512 in SEQ ID NO: 2; V at a residue corresponding to position 513 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; L, M at a residue corresponding to position 526 in SEQ ID NO: 2; L at a residue corresponding to position 527 in SEQ ID NO: 2; R, H at a residue corresponding to position 532 in SEQ ID NO: 2; L at a residue corresponding to position 535 in SEQ ID NO: 2; Q at a residue corresponding to position 540 in SEQ ID NO: 2; Y, W at a residue corresponding to position 542 in SEQ ID NO: 2; N at a residue corresponding to position 546 in SEQ ID NO: 2; Q at a residue corresponding to position 552 in SEQ ID NO: 2; E at a residue corresponding to position 554 in SEQ ID NO: 2; F at a residue corresponding to position 558 in SEQ ID NO: 2; D at a residue corresponding to position 560 in SEQ ID NO: 2; D at a residue corresponding to position 561 in SEQ ID NO: 2; F, L, W at a residue corresponding to position 563 in SEQ ID NO: 2; D, K, S, L, T, R at a residue corresponding to position 564 in SEQ ID NO: 2; M, I, V, P, T at a residue corresponding to position 565 in SEQ ID NO: 2; A, E, D, P at a residue corresponding to position 566 in SEQ ID NO: 2; K, Q, T, N, S, A at a residue corresponding to position 567 in SEQ ID NO: 2; G, N, D at a residue corresponding to position 568 in SEQ ID NO: 2; M, K at a residue corresponding to position 569 in SEQ ID NO: 2; V, L at a residue corresponding to position 570 in SEQ ID NO: 2; R, P at a residue corresponding to position 572 in SEQ ID NO: 2; T, S at a residue corresponding to position 573 in SEQ ID NO: 2; M, L at a residue corresponding to position 574 in SEQ ID NO: 2; A, Y, K, T at a residue corresponding to position 575 in SEQ ID NO: 2; Y, A, L at a residue corresponding to position 577 in SEQ ID NO: 2; Q, A at a residue corresponding to position 578 in SEQ ID NO: 2; D at a residue corresponding to position 583 in SEQ ID NO: 2; N at a residue corresponding to position 584 in SEQ ID NO: 2; H, L at a residue corresponding to position 585 in SEQ ID NO: 2; A at a residue corresponding to position 587 in SEQ ID NO: 2; Q at a residue corresponding to position 588 in SEQ ID NO: 2; Q, E at a residue corresponding to position 591 in SEQ ID NO: 2; A at a residue corresponding to position 593 in SEQ ID NO: 2; E at a residue corresponding to position 595 in SEQ ID NO: 2; K at a residue corresponding to position 596 in SEQ ID NO: 2; D at a residue corresponding to position 598 in SEQ ID NO: 2; S at a residue corresponding to position 599 in SEQ ID NO: 2; L at a residue corresponding to position 601 in SEQ ID NO: 2; more than one of the foregoing; or any combination thereof

In some embodiments, provided herein is microbial organism (e.g., host microbial organism) that has a recombinant nucleic acid encoding an engineered ACL having: E, R at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; D, N, R, P, K at a residue corresponding to position 10 in SEQ ID NO: 2; A, V, M, I at a residue corresponding to position 12 in SEQ ID NO: 2; C at a residue corresponding to position 13 in SEQ ID NO: 2; Q, P, A at a residue corresponding to position 14 in SEQ ID NO: 2; S, E at a residue corresponding to position 15 in SEQ ID NO: 2; I, C at a residue corresponding to position 16 in SEQ ID NO: 2; D, R at a residue corresponding to position 19 in SEQ ID NO: 2; H, K, R, D at a residue corresponding to position 20 in SEQ ID NO: 2; E, D at a residue corresponding to position 22 in SEQ ID NO: 2; N at a residue corresponding to position 25 in SEQ ID NO: 2; M at a residue corresponding to position 28 in SEQ ID NO: 2; C at a residue corresponding to position 37 in SEQ ID NO: 2; R at a residue corresponding to position 39 in SEQ ID NO: 2; T at a residue corresponding to position 40 in SEQ ID NO: 2; V, I, M at a residue corresponding to position 41 in SEQ ID NO: 2; S at a residue corresponding to position 43 in SEQ ID NO: 2; I, V at a residue corresponding to position 45 in SEQ ID NO: 2; R, L, I, V, F at a residue corresponding to position 50 in SEQ ID NO: 2; R at a residue corresponding to position 51 in SEQ ID NO: 2; L at a residue corresponding to position 53 in SEQ ID NO: 2; F at a residue corresponding to position 57 in SEQ ID NO: 2; F, M, L, V at a residue corresponding to position 58 in SEQ ID NO: 2; V at a residue corresponding to position 59 in SEQ ID NO: 2; P at a residue corresponding to position 61 in SEQ ID NO: 2; D at a residue corresponding to position 62 in SEQ ID NO: 2; D at a residue corresponding to position 63 in SEQ ID NO: 2; K at a residue corresponding to position 67 in SEQ ID NO: 2; F, L, Y at a residue corresponding to position 69 in SEQ ID NO: 2; W, F at a residue corresponding to position 71 in SEQ ID NO: 2; A, R at a residue corresponding to position 72 in SEQ ID NO: 2; F at a residue corresponding to position 74 in SEQ ID NO: 2; D at a residue corresponding to position 75 in SEQ ID NO: 2; D, T, A, R at a residue corresponding to position 76 in SEQ ID NO: 2; R at a residue corresponding to position 77 in SEQ ID NO: 2; A at a residue corresponding to position 78 in SEQ ID NO: 2; K at a residue corresponding to position 80 in SEQ ID NO: 2; C at a residue corresponding to position 81 in SEQ ID NO: 2; S, N at a residue corresponding to position 83 in SEQ ID NO: 2; Y, H at a residue corresponding to position 84 in SEQ ID NO: 2; F at a residue corresponding to position 85 in SEQ ID NO: 2; Q, A at a residue corresponding to position 86 in SEQ ID NO: 2; R at a residue corresponding to position 87 in SEQ ID NO: 2; L at a residue corresponding to position 88 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; A at a residue corresponding to position 97 in SEQ ID NO: 2; M at a residue corresponding to position 98 in SEQ ID NO: 2; L at a residue corresponding to position 99 in SEQ ID NO: 2; C at a residue corresponding to position 100 in SEQ ID NO: 2; P at a residue corresponding to position 101 in SEQ ID NO: 2; R at a residue corresponding to position 103 in SEQ ID NO: 2; A, L, W at a residue corresponding to position 104 in SEQ ID NO: 2; F at a residue corresponding to position 106 in SEQ ID NO: 2; V at a residue corresponding to position 108 in SEQ ID NO: 2; V at a residue corresponding to position 109 in SEQ ID NO: 2; A, M at a residue corresponding to position 110 in SEQ ID NO: 2; C at a residue corresponding to position 113 in SEQ ID NO: 2; I at a residue corresponding to position 116 in SEQ ID NO: 2; T, V, I at a residue corresponding to position 119 in SEQ ID NO: 2; Y, V, F, L, M, A at a residue corresponding to position 120 in SEQ ID NO: 2; T at a residue corresponding to position 121 in SEQ ID NO: 2; I, M, V at a residue corresponding to position 123 in SEQ ID NO: 2; S, N, Y at a residue corresponding to position 124 in SEQ ID NO: 2; P, T, W at a residue corresponding to position 125 in SEQ ID NO: 2; R, A, N, M at a residue corresponding to position 126 in SEQ ID NO: 2; L, W, F at a residue corresponding to position 127 in SEQ ID NO: 2; R at a residue corresponding to position 128 in SEQ ID NO: 2; M at a residue corresponding to position 130 in SEQ ID NO: 2; E at a residue corresponding to position 131 in SEQ ID NO: 2; E, K, Q at a residue corresponding to position 136 in SEQ ID NO: 2; M at a residue corresponding to position 137 in SEQ ID NO: 2; M, I, C at a residue corresponding to position 140 in SEQ ID NO: 2; M at a residue corresponding to position 141 in SEQ ID NO: 2; Q, N at a residue corresponding to position 142 in SEQ ID NO: 2; R, D, G at a residue corresponding to position 145 in SEQ ID NO: 2; S at a residue corresponding to position 147 in SEQ ID NO: 2; V, L, A at a residue corresponding to position 148 in SEQ ID NO: 2; V at a residue corresponding to position 149 in SEQ ID NO: 2; V at a residue corresponding to position 150 in SEQ ID NO: 2; C at a residue corresponding to position 151 in SEQ ID NO: 2; E, R at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 165 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, L, V at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 189 in SEQ ID NO: 2; V at a residue corresponding to position 190 in SEQ ID NO: 2; E, A at a residue corresponding to position 191 in SEQ ID NO: 2; T at a residue corresponding to position 192 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; P at a residue corresponding to position 196 in SEQ ID NO: 2; L, A at a residue corresponding to position 201 in SEQ ID NO: 2; T at a residue corresponding to position 205 in SEQ ID NO: 2; D at a residue corresponding to position 208 in SEQ ID NO: 2; I, T, V at a residue corresponding to position 209 in SEQ ID NO: 2; L at a residue corresponding to position 211 in SEQ ID NO: 2; L, M at a residue corresponding to position 212 in SEQ ID NO: 2; I, M, V at a residue corresponding to position 213 in SEQ ID NO: 2; Y at a residue corresponding to position 214 in SEQ ID NO: 2; S at a residue corresponding to position 215 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; K, L, T, H, R, M at a residue corresponding to position 221 in SEQ ID NO: 2; S at a residue corresponding to position 222 in SEQ ID NO: 2; A at a residue corresponding to position 224 in SEQ ID NO: 2; M, V, I at a residue corresponding to position 226 in SEQ ID NO: 2; I at a residue corresponding to position 227 in SEQ ID NO: 2; S at a residue corresponding to position 228 in SEQ ID NO: 2; H, K at a residue corresponding to position 230 in SEQ ID NO: 2; L at a residue corresponding to position 232 in SEQ ID NO: 2; A at a residue corresponding to position 233 in SEQ ID NO: 2; S at a residue corresponding to position 234 in SEQ ID NO: 2; Q at a residue corresponding to position 235 in SEQ ID NO: 2; M at a residue corresponding to position 236 in SEQ ID NO: 2; A, M, Q at a residue corresponding to position 237 in SEQ ID NO: 2; M, H at a residue corresponding to position 238 in SEQ ID NO: 2; V, L, M at a residue corresponding to position 239 in SEQ ID NO: 2; A at a residue corresponding to position 240 in SEQ ID NO: 2; Q, E, H, A at a residue corresponding to position 241 in SEQ ID NO: 2; C, V, T at a residue corresponding to position 242 in SEQ ID NO: 2; W, F, M, I, V at a residue corresponding to position 243 in SEQ ID NO: 2; P, Q, G, H, E at a residue corresponding to position 244 in SEQ ID NO: 2; L, M, W at a residue corresponding to position 245 in SEQ ID NO: 2; I at a residue corresponding to position 246 in SEQ ID NO: 2; R, P at a residue corresponding to position 247 in SEQ ID NO: 2; V, H at a residue corresponding to position 252 in SEQ ID NO: 2; L, V, M at a residue corresponding to position 253 in SEQ ID NO: 2; M, I, V at a residue corresponding to position 254 in SEQ ID NO: 2; C, V, T, N at a residue corresponding to position 255 in SEQ ID NO: 2; Y, V, F at a residue corresponding to position 256 in SEQ ID NO: 2; S, H, F, Y at a residue corresponding to position 260 in SEQ ID NO: 2; T, C, A at a residue corresponding to position 262 in SEQ ID NO: 2; Y at a residue corresponding to position 263 in SEQ ID NO: 2; A at a residue corresponding to position 264 in SEQ ID NO: 2; M at a residue corresponding to position 265 in SEQ ID NO: 2; N, M at a residue corresponding to position 266 in SEQ ID NO: 2; M, F, L, W, G at a residue corresponding to position 267 in SEQ ID NO: 2; M, A at a residue corresponding to position 268 in SEQ ID NO: 2; M at a residue corresponding to position 270 in SEQ ID NO: 2; L, C, A at a residue corresponding to position 271 in SEQ ID NO: 2; A, V, T, P, C at a residue corresponding to position 272 in SEQ ID NO: 2; I, V, M, F at a residue corresponding to position 273 in SEQ ID NO: 2; C, M, H, R, Y at a residue corresponding to position 274 in SEQ ID NO: 2; H, C at a residue corresponding to position 275 in SEQ ID NO: 2; A at a residue corresponding to position 277 in SEQ ID NO: 2; T, A, C at a residue corresponding to position 278 in SEQ ID NO: 2; M, I, V at a residue corresponding to position 279 in SEQ ID NO: 2; V, L at a residue corresponding to position 281 in SEQ ID NO: 2; E, D, P, R at a residue corresponding to position 283 in SEQ ID NO: 2; R, F, D at a residue corresponding to position 285 in SEQ ID NO: 2; D, F at a residue corresponding to position 286 in SEQ ID NO: 2; A, P, V at a residue corresponding to position 287 in SEQ ID NO: 2; A at a residue corresponding to position 290 in SEQ ID NO: 2; M, I, L, V at a residue corresponding to position 291 in SEQ ID NO: 2; H at a residue corresponding to position 292 in SEQ ID NO: 2; A at a residue corresponding to position 294 in SEQ ID NO: 2; V, I, M at a residue corresponding to position 295 in SEQ ID NO: 2; Q, D at a residue corresponding to position 296 in SEQ ID NO: 2; M, V, L at a residue corresponding to position 298 in SEQ ID NO: 2; A at a residue corresponding to position 299 in SEQ ID NO: 2; Q at a residue corresponding to position 300 in SEQ ID NO: 2; Q at a residue corresponding to position 302 in SEQ ID NO: 2; A, P, C, V at a residue corresponding to position 303 in SEQ ID NO: 2; S at a residue corresponding to position 304 in SEQ ID NO: 2; V, M, I at a residue corresponding to position 305 in SEQ ID NO: 2; I, V, Y at a residue corresponding to position 306 in SEQ ID NO: 2; M, W, P at a residue corresponding to position 307 in SEQ ID NO: 2; G at a residue corresponding to position 308 in SEQ ID NO: 2; T, A, R at a residue corresponding to position 311 in SEQ ID NO: 2; F, V, M at a residue corresponding to position 312 in SEQ ID NO: 2; F, W at a residue corresponding to position 313 in SEQ ID NO: 2; Q, H, E at a residue corresponding to position 314 in SEQ ID NO: 2; A, K, L, R at a residue corresponding to position 315 in SEQ ID NO: 2; I, M at a residue corresponding to position 316 in SEQ ID NO: 2; D, H at a residue corresponding to position 318 in SEQ ID NO: 2; R, Q at a residue corresponding to position 321 in SEQ ID NO: 2; A, K, H, Q at a residue corresponding to position 322 in SEQ ID NO: 2; F at a residue corresponding to position 330 in SEQ ID NO: 2; G at a residue corresponding to position 331 in SEQ ID NO: 2; S at a residue corresponding to position 333 in SEQ ID NO: 2; V, I, M at a residue corresponding to position 334 in SEQ ID NO: 2; K at a residue corresponding to position 335 in SEQ ID NO: 2; V, T, M, Y, W, I, F at a residue corresponding to position 336 in SEQ ID NO: 2; L, V, M at a residue corresponding to position 337 in SEQ ID NO: 2; L, M, V, W at a residue corresponding to position 338 in SEQ ID NO: 2; T, S, F at a residue corresponding to position 339 in SEQ ID NO: 2; G at a residue corresponding to position 340 in SEQ ID NO: 2; S at a residue corresponding to position 342 in SEQ ID NO: 2; P, A at a residue corresponding to position 343 in SEQ ID NO: 2; I, C, M, V at a residue corresponding to position 344 in SEQ ID NO: 2; S at a residue corresponding to position 345 in SEQ ID NO: 2; E, V, A, P at a residue corresponding to position 346 in SEQ ID NO: 2; E at a residue corresponding to position 347 in SEQ ID NO: 2; I, L, T at a residue corresponding to position 348 in SEQ ID NO: 2; Y, F, L at a residue corresponding to position 349 in SEQ ID NO: 2; R, Q, W at a residue corresponding to position 351 in SEQ ID NO: 2; I, M at a residue corresponding to position 352 in SEQ ID NO: 2; R at a residue corresponding to position 353 in SEQ ID NO: 2; K at a residue corresponding to position 354 in SEQ ID NO: 2; A at a residue corresponding to position 355 in SEQ ID NO: 2; F, I, V, A, G, L, W at a residue corresponding to position 357 in SEQ ID NO: 2; Q at a residue corresponding to position 358 in SEQ ID NO: 2; Q at a residue corresponding to position 359 in SEQ ID NO: 2; C, H, L at a residue corresponding to position 362 in SEQ ID NO: 2; V, I, G at a residue corresponding to position 363 in SEQ ID NO: 2; M, I, V at a residue corresponding to position 365 in SEQ ID NO: 2; I, L, F, V at a residue corresponding to position 366 in SEQ ID NO: 2; V, I, L, M, T, C at a residue corresponding to position 367 in SEQ ID NO: 2; N at a residue corresponding to position 368 in SEQ ID NO: 2; A, G at a residue corresponding to position 369 in SEQ ID NO: 2; Y at a residue corresponding to position 370 in SEQ ID NO: 2; C, A at a residue corresponding to position 375 in SEQ ID NO: 2; G, S at a residue corresponding to position 376 in SEQ ID NO: 2; G at a residue corresponding to position 377 in SEQ ID NO: 2; T at a residue corresponding to position 378 in SEQ ID NO: 2; V, C, L, A, I at a residue corresponding to position 379 in SEQ ID NO: 2; S, V, H, T at a residue corresponding to position 380 in SEQ ID NO: 2; L, C at a residue corresponding to position 381 in SEQ ID NO: 2; T, C, N, L, P at a residue corresponding to position 382 in SEQ ID NO: 2; Y, R at a residue corresponding to position 383 in SEQ ID NO: 2; N, W, D at a residue corresponding to position 384 in SEQ ID NO: 2; G at a residue corresponding to position 385 in SEQ ID NO: 2; V, T, L at a residue corresponding to position 386 in SEQ ID NO: 2; R, H, N, D, E at a residue corresponding to position 387 in SEQ ID NO: 2; K, H at a residue corresponding to position 388 in SEQ ID NO: 2; A, P, L at a residue corresponding to position 389 in SEQ ID NO: 2; S, T at a residue corresponding to position 391 in SEQ ID NO: 2; V at a residue corresponding to position 392 in SEQ ID NO: 2; I, K at a residue corresponding to position 394 in SEQ ID NO: 2; V at a residue corresponding to position 396 in SEQ ID NO: 2; H at a residue corresponding to position 398 in SEQ ID NO: 2; C, I, T, V at a residue corresponding to position 399 in SEQ ID NO: 2; Q, D at a residue corresponding to position 400 in SEQ ID NO: 2; L, I, C, A, M at a residue corresponding to position 401 in SEQ ID NO: 2; V at a residue corresponding to position 404 in SEQ ID NO: 2; E at a residue corresponding to position 406 in SEQ ID NO: 2; P at a residue corresponding to position 407 in SEQ ID NO: 2; G, Q, E at a residue corresponding to position 409 in SEQ ID NO: 2; S at a residue corresponding to position 411 in SEQ ID NO: 2; L, C at a residue corresponding to position 414 in SEQ ID NO: 2; R at a residue corresponding to position 416 in SEQ ID NO: 2; Q, H, M, S at a residue corresponding to position 419 in SEQ ID NO: 2; V at a residue corresponding to position 420 in SEQ ID NO: 2; F, T at a residue corresponding to position 421 in SEQ ID NO: 2; L, H at a residue corresponding to position 425 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; V, T at a residue corresponding to position 434 in SEQ ID NO: 2; M at a residue corresponding to position 435 in SEQ ID NO: 2; F, M at a residue corresponding to position 441 in SEQ ID NO: 2; Y, R, K, C, H at a residue corresponding to position 442 in SEQ ID NO: 2; S at a residue corresponding to position 443 in SEQ ID NO: 2; L, V, M, I at a residue corresponding to position 446 in SEQ ID NO: 2; A at a residue corresponding to position 447 in SEQ ID NO: 2; M, D at a residue corresponding to position 450 in SEQ ID NO: 2; E at a residue corresponding to position 451 in SEQ ID NO: 2; P at a residue corresponding to position 452 in SEQ ID NO: 2; G at a residue corresponding to position 453 in SEQ ID NO: 2; N, D at a residue corresponding to position 456 in SEQ ID NO: 2; F, I, R, W, Y at a residue corresponding to position 460 in SEQ ID NO: 2; Y, L at a residue corresponding to position 461 in SEQ ID NO: 2; V, I at a residue corresponding to position 462 in SEQ ID NO: 2; D at a residue corresponding to position 463 in SEQ ID NO: 2; L at a residue corresponding to position 465 in SEQ ID NO: 2; A, D, K at a residue corresponding to position 466 in SEQ ID NO: 2; K at a residue corresponding to position 467 in SEQ ID NO: 2; M at a residue corresponding to position 468 in SEQ ID NO: 2; L at a residue corresponding to position 469 in SEQ ID NO: 2; V at a residue corresponding to position 470 in SEQ ID NO: 2; T, V at a residue corresponding to position 471 in SEQ ID NO: 2; N, S at a residue corresponding to position 473 in SEQ ID NO: 2; Y, F, V, L, I, N at a residue corresponding to position 476 in SEQ ID NO: 2; T at a residue corresponding to position 478 in SEQ ID NO: 2; V, P at a residue corresponding to position 479 in SEQ ID NO: 2; S, A, P, L at a residue corresponding to position 480 in SEQ ID NO: 2; P, E at a residue corresponding to position 481 in SEQ ID NO: 2; L, E, R, A at a residue corresponding to position 485 in SEQ ID NO: 2; C at a residue corresponding to position 490 in SEQ ID NO: 2; P at a residue corresponding to position 492 in SEQ ID NO: 2; C at a residue corresponding to position 498 in SEQ ID NO: 2; V at a residue corresponding to position 507 in SEQ ID NO: 2; V at a residue corresponding to position 508 in SEQ ID NO: 2; A at a residue corresponding to position 510 in SEQ ID NO: 2; V, L at a residue corresponding to position 511 in SEQ ID NO: 2; I at a residue corresponding to position 512 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; M, L at a residue corresponding to position 526 in SEQ ID NO: 2; L at a residue corresponding to position 527 in SEQ ID NO: 2; R, H at a residue corresponding to position 532 in SEQ ID NO: 2; L at a residue corresponding to position 535 in SEQ ID NO: 2; Q at a residue corresponding to position 540 in SEQ ID NO: 2; Y, W at a residue corresponding to position 542 in SEQ ID NO: 2; N at a residue corresponding to position 546 in SEQ ID NO: 2; Q at a residue corresponding to position 552 in SEQ ID NO: 2; E at a residue corresponding to position 554 in SEQ ID NO: 2; D at a residue corresponding to position 560 in SEQ ID NO: 2; D at a residue corresponding to position 561 in SEQ ID NO: 2; L, F at a residue corresponding to position 563 in SEQ ID NO: 2; S, T, L, D, R, K at a residue corresponding to position 564 in SEQ ID NO: 2; T, M, P, V, I at a residue corresponding to position 565 in SEQ ID NO: 2; D, A, E, P at a residue corresponding to position 566 in SEQ ID NO: 2; A, S, Q, N, T, K at a residue corresponding to position 567 in SEQ ID NO: 2; G, D, N at a residue corresponding to position 568 in SEQ ID NO: 2; M, K at a residue corresponding to position 569 in SEQ ID NO: 2; V, L at a residue corresponding to position 570 in SEQ ID NO: 2; P at a residue corresponding to position 572 in SEQ ID NO: 2; S, T at a residue corresponding to position 573 in SEQ ID NO: 2; M, L at a residue corresponding to position 574 in SEQ ID NO: 2; T, Y at a residue corresponding to position 575 in SEQ ID NO: 2; A, L at a residue corresponding to position 577 in SEQ ID NO: 2; A, Q at a residue corresponding to position 578 in SEQ ID NO: 2; D at a residue corresponding to position 583 in SEQ ID NO: 2; N at a residue corresponding to position 584 in SEQ ID NO: 2; H at a residue corresponding to position 585 in SEQ ID NO: 2; A at a residue corresponding to position 587 in SEQ ID NO: 2; Q at a residue corresponding to position 588 in SEQ ID NO: 2; Q, E at a residue corresponding to position 591 in SEQ ID NO: 2; A at a residue corresponding to position 593 in SEQ ID NO: 2; E at a residue corresponding to position 595 in SEQ ID NO: 2; K at a residue corresponding to position 596 in SEQ ID NO: 2; D at a residue corresponding to position 598 in SEQ ID NO: 2; S at a residue corresponding to position 599 in SEQ ID NO: 2; L at a residue corresponding to position 601 in SEQ ID NO: 2; or any combination thereof.

In some embodiments, provided herein is microbial organism (e.g., host microbial organism) that has a recombinant nucleic acid encoding an engineered ACL having: E, R at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; D, N, R, P at a residue corresponding to position 10 in SEQ ID NO: 2; A, V, M at a residue corresponding to position 12 in SEQ ID NO: 2; Q, P at a residue corresponding to position 14 in SEQ ID NO: 2; S, E at a residue corresponding to position 15 in SEQ ID NO: 2; I at a residue corresponding to position 16 in SEQ ID NO: 2; D, R at a residue corresponding to position 19 in SEQ ID NO: 2; H, K, R, D at a residue corresponding to position 20 in SEQ ID NO: 2; E, D at a residue corresponding to position 22 in SEQ ID NO: 2; M at a residue corresponding to position 28 in SEQ ID NO: 2; R at a residue corresponding to position 39 in SEQ ID NO: 2; V at a residue corresponding to position 41 in SEQ ID NO: 2; S at a residue corresponding to position 43 in SEQ ID NO: 2; I at a residue corresponding to position 45 in SEQ ID NO: 2; R, L, I, V, F at a residue corresponding to position 50 in SEQ ID NO: 2; F at a residue corresponding to position 57 in SEQ ID NO: 2; F, M, L, V at a residue corresponding to position 58 in SEQ ID NO: 2; P at a residue corresponding to position 61 in SEQ ID NO: 2; D at a residue corresponding to position 62 in SEQ ID NO: 2; D at a residue corresponding to position 63 in SEQ ID NO: 2; K at a residue corresponding to position 67 in SEQ ID NO: 2; F, L, Y at a residue corresponding to position 69 in SEQ ID NO: 2; W, F at a residue corresponding to position 71 in SEQ ID NO: 2; A, R at a residue corresponding to position 72 in SEQ ID NO: 2; D, T, A, R at a residue corresponding to position 76 in SEQ ID NO: 2; R at a residue corresponding to position 77 in SEQ ID NO: 2; A at a residue corresponding to position 78 in SEQ ID NO: 2; C at a residue corresponding to position 81 in SEQ ID NO: 2; Y at a residue corresponding to position 84 in SEQ ID NO: 2; F at a residue corresponding to position 85 in SEQ ID NO: 2; Q at a residue corresponding to position 86 in SEQ ID NO: 2; R at a residue corresponding to position 87 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; M at a residue corresponding to position 98 in SEQ ID NO: 2; A at a residue corresponding to position 104 in SEQ ID NO: 2; V at a residue corresponding to position 108 in SEQ ID NO: 2; V at a residue corresponding to position 109 in SEQ ID NO: 2; C at a residue corresponding to position 113 in SEQ ID NO: 2; I at a residue corresponding to position 116 in SEQ ID NO: 2; T at a residue corresponding to position 119 in SEQ ID NO: 2; Y, V, F, L, M, A at a residue corresponding to position 120 in SEQ ID NO: 2; I at a residue corresponding to position 123 in SEQ ID NO: 2; S, N at a residue corresponding to position 124 in SEQ ID NO: 2; P, T, W at a residue corresponding to position 125 in SEQ ID NO: 2; R, A, N at a residue corresponding to position 126 in SEQ ID NO: 2; L, W at a residue corresponding to position 127 in SEQ ID NO: 2; M at a residue corresponding to position 130 in SEQ ID NO: 2; E at a residue corresponding to position 131 in SEQ ID NO: 2; E, K at a residue corresponding to position 136 in SEQ ID NO: 2; M, I, C at a residue corresponding to position 140 in SEQ ID NO: 2; M at a residue corresponding to position 141 in SEQ ID NO: 2; Q, N at a residue corresponding to position 142 in SEQ ID NO: 2; R at a residue corresponding to position 145 in SEQ ID NO: 2; S at a residue corresponding to position 147 in SEQ ID NO: 2; V at a residue corresponding to position 148 in SEQ ID NO: 2; V at a residue corresponding to position 149 in SEQ ID NO: 2; C at a residue corresponding to position 151 in SEQ ID NO: 2; E, R at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, L, V at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 190 in SEQ ID NO: 2; E, A at a residue corresponding to position 191 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; L, A at a residue corresponding to position 201 in SEQ ID NO: 2; T at a residue corresponding to position 205 in SEQ ID NO: 2; I at a residue corresponding to position 209 in SEQ ID NO: 2; Y at a residue corresponding to position 214 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; K, L, T, H at a residue corresponding to position 221 in SEQ ID NO: 2; M, V at a residue corresponding to position 226 in SEQ ID NO: 2; S at a residue corresponding to position 228 in SEQ ID NO: 2; H, K at a residue corresponding to position 230 in SEQ ID NO: 2; L at a residue corresponding to position 232 in SEQ ID NO: 2; S at a residue corresponding to position 234 in SEQ ID NO: 2; A at a residue corresponding to position 237 in SEQ ID NO: 2; M at a residue corresponding to position 238 in SEQ ID NO: 2; V at a residue corresponding to position 239 in SEQ ID NO: 2; Q, E at a residue corresponding to position 241 in SEQ ID NO: 2; C, V, T at a residue corresponding to position 242 in SEQ ID NO: 2; W, F, M at a residue corresponding to position 243 in SEQ ID NO: 2; P, Q at a residue corresponding to position 244 in SEQ ID NO: 2; I at a residue corresponding to position 246 in SEQ ID NO: 2; R at a residue corresponding to position 247 in SEQ ID NO: 2; L, V at a residue corresponding to position 253 in SEQ ID NO: 2; M, I, V at a residue corresponding to position 254 in SEQ ID NO: 2; S, H at a residue corresponding to position 260 in SEQ ID NO: 2; T at a residue corresponding to position 262 in SEQ ID NO: 2; Y at a residue corresponding to position 263 in SEQ ID NO: 2; N at a residue corresponding to position 266 in SEQ ID NO: 2; M, F at a residue corresponding to position 267 in SEQ ID NO: 2; A at a residue corresponding to position 272 in SEQ ID NO: 2; I, V, M, F at a residue corresponding to position 273 in SEQ ID NO: 2; C at a residue corresponding to position 274 in SEQ ID NO: 2; T, A at a residue corresponding to position 278 in SEQ ID NO: 2; M at a residue corresponding to position 279 in SEQ ID NO: 2; V at a residue corresponding to position 281 in SEQ ID NO: 2; E at a residue corresponding to position 283 in SEQ ID NO: 2; R at a residue corresponding to position 285 in SEQ ID NO: 2; A at a residue corresponding to position 290 in SEQ ID NO: 2; M, I, L, V at a residue corresponding to position 291 in SEQ ID NO: 2; H at a residue corresponding to position 292 in SEQ ID NO: 2; V, I, M at a residue corresponding to position 295 in SEQ ID NO: 2; Q at a residue corresponding to position 296 in SEQ ID NO: 2; M, V at a residue corresponding to position 298 in SEQ ID NO: 2; Q at a residue corresponding to position 300 in SEQ ID NO: 2; Q at a residue corresponding to position 302 in SEQ ID NO: 2; A, P at a residue corresponding to position 303 in SEQ ID NO: 2; S at a residue corresponding to position 304 in SEQ ID NO: 2; V, M, I at a residue corresponding to position 305 in SEQ ID NO: 2; M at a residue corresponding to position 307 in SEQ ID NO: 2; T, A at a residue corresponding to position 311 in SEQ ID NO: 2; F at a residue corresponding to position 313 in SEQ ID NO: 2; Q, H at a residue corresponding to position 314 in SEQ ID NO: 2; A, K, L, R at a residue corresponding to position 315 in SEQ ID NO: 2; I at a residue corresponding to position 316 in SEQ ID NO: 2; D, H at a residue corresponding to position 318 in SEQ ID NO: 2; R, Q at a residue corresponding to position 321 in SEQ ID NO: 2; A, K, H, Q at a residue corresponding to position 322 in SEQ ID NO: 2; F at a residue corresponding to position 330 in SEQ ID NO: 2; S at a residue corresponding to position 333 in SEQ ID NO: 2; V, I at a residue corresponding to position 334 in SEQ ID NO: 2; V, T, M, Y at a residue corresponding to position 336 in SEQ ID NO: 2; L, V at a residue corresponding to position 337 in SEQ ID NO: 2; L, M, V at a residue corresponding to position 338 in SEQ ID NO: 2; T at a residue corresponding to position 339 in SEQ ID NO: 2; G at a residue corresponding to position 340 in SEQ ID NO: 2; I, C, M at a residue corresponding to position 344 in SEQ ID NO: 2; S at a residue corresponding to position 345 in SEQ ID NO: 2; E at a residue corresponding to position 346 in SEQ ID NO: 2; E at a residue corresponding to position 347 in SEQ ID NO: 2; I, L, T at a residue corresponding to position 348 in SEQ ID NO: 2; Y, F at a residue corresponding to position 349 in SEQ ID NO: 2; R, Q at a residue corresponding to position 351 in SEQ ID NO: 2; I at a residue corresponding to position 352 in SEQ ID NO: 2; R at a residue corresponding to position 353 in SEQ ID NO: 2; K at a residue corresponding to position 354 in SEQ ID NO: 2; A at a residue corresponding to position 355 in SEQ ID NO: 2; F, I, V, A, G, L, W at a residue corresponding to position 357 in SEQ ID NO: 2; Q at a residue corresponding to position 358 in SEQ ID NO: 2; Q at a residue corresponding to position 359 in SEQ ID NO: 2; C, H at a residue corresponding to position 362 in SEQ ID NO: 2; V, I at a residue corresponding to position 363 in SEQ ID NO: 2; M at a residue corresponding to position 365 in SEQ ID NO: 2; V, I, L, M at a residue corresponding to position 367 in SEQ ID NO: 2; A at a residue corresponding to position 369 in SEQ ID NO: 2; C at a residue corresponding to position 375 in SEQ ID NO: 2; G, S at a residue corresponding to position 376 in SEQ ID NO: 2; V, C, L at a residue corresponding to position 379 in SEQ ID NO: 2; T, C at a residue corresponding to position 382 in SEQ ID NO: 2; Y at a residue corresponding to position 383 in SEQ ID NO: 2; N at a residue corresponding to position 384 in SEQ ID NO: 2; V, T at a residue corresponding to position 386 in SEQ ID NO: 2; R, H, N, D at a residue corresponding to position 387 in SEQ ID NO: 2; K, H at a residue corresponding to position 388 in SEQ ID NO: 2; A, P, L at a residue corresponding to position 389 in SEQ ID NO: 2; C, I, T, V at a residue corresponding to position 399 in SEQ ID NO: 2; Q at a residue corresponding to position 400 in SEQ ID NO: 2; L, I, C at a residue corresponding to position 401 in SEQ ID NO: 2; V at a residue corresponding to position 404 in SEQ ID NO: 2; P at a residue corresponding to position 407 in SEQ ID NO: 2; G at a residue corresponding to position 409 in SEQ ID NO: 2; R at a residue corresponding to position 416 in SEQ ID NO: 2; Q at a residue corresponding to position 419 in SEQ ID NO: 2; V at a residue corresponding to position 420 in SEQ ID NO: 2; F at a residue corresponding to position 421 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; V at a residue corresponding to position 434 in SEQ ID NO: 2; F at a residue corresponding to position 441 in SEQ ID NO: 2; Y, R, K, C, H at a residue corresponding to position 442 in SEQ ID NO: 2; L, V, M, I at a residue corresponding to position 446 in SEQ ID NO: 2; M at a residue corresponding to position 450 in SEQ ID NO: 2; P at a residue corresponding to position 452 in SEQ ID NO: 2; G at a residue corresponding to position 453 in SEQ ID NO: 2; N, D at a residue corresponding to position 456 in SEQ ID NO: 2; F, I, R at a residue corresponding to position 460 in SEQ ID NO: 2; Y at a residue corresponding to position 461 in SEQ ID NO: 2; L at a residue corresponding to position 465 in SEQ ID NO: 2; A, D at a residue corresponding to position 466 in SEQ ID NO: 2; T at a residue corresponding to position 471 in SEQ ID NO: 2; N, S at a residue corresponding to position 473 in SEQ ID NO: 2; Y, F, V, L, I at a residue corresponding to position 476 in SEQ ID NO: 2; V, P at a residue corresponding to position 479 in SEQ ID NO: 2; S, A, P at a residue corresponding to position 480 in SEQ ID NO: 2; P, E at a residue corresponding to position 481 in SEQ ID NO: 2; L, E, R, A at a residue corresponding to position 485 in SEQ ID NO: 2; C at a residue corresponding to position 490 in SEQ ID NO: 2; P at a residue corresponding to position 492 in SEQ ID NO: 2; V at a residue corresponding to position 507 in SEQ ID NO: 2; V at a residue corresponding to position 511 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; M, L at a residue corresponding to position 526 in SEQ ID NO: 2; L at a residue corresponding to position 527 in SEQ ID NO: 2; R, H at a residue corresponding to position 532 in SEQ ID NO: 2; Q at a residue corresponding to position 540 in SEQ ID NO: 2; Y at a residue corresponding to position 542 in SEQ ID NO: 2; N at a residue corresponding to position 546 in SEQ ID NO: 2; E at a residue corresponding to position 554 in SEQ ID NO: 2; D at a residue corresponding to position 561 in SEQ ID NO: 2; S, T at a residue corresponding to position 564 in SEQ ID NO: 2; T, M, P, V, I at a residue corresponding to position 565 in SEQ ID NO: 2; D at a residue corresponding to position 566 in SEQ ID NO: 2; A, S, Q, N at a residue corresponding to position 567 in SEQ ID NO: 2; G, D, N at a residue corresponding to position 568 in SEQ ID NO: 2; V at a residue corresponding to position 570 in SEQ ID NO: 2; P at a residue corresponding to position 572 in SEQ ID NO: 2; M at a residue corresponding to position 574 in SEQ ID NO: 2; T at a residue corresponding to position 575 in SEQ ID NO: 2; A at a residue corresponding to position 577 in SEQ ID NO: 2; N at a residue corresponding to position 584 in SEQ ID NO: 2; A at a residue corresponding to position 587 in SEQ ID NO: 2; E at a residue corresponding to position 595 in SEQ ID NO: 2; K at a residue corresponding to position 596 in SEQ ID NO: 2; S at a residue corresponding to position 599 in SEQ ID NO: 2; or any combination thereof.

In some embodiments, provided herein is microbial organism (e.g., host microbial organism) that has a recombinant nucleic acid encoding an engineered ACL having: E, R at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; D, N at a residue corresponding to position 10 in SEQ ID NO: 2; A at a residue corresponding to position 12 in SEQ ID NO: 2; Q, P at a residue corresponding to position 14 in SEQ ID NO: 2; S, E at a residue corresponding to position 15 in SEQ ID NO: 2; D at a residue corresponding to position 19 in SEQ ID NO: 2; H, K, R at a residue corresponding to position 20 in SEQ ID NO: 2; E at a residue corresponding to position 22 in SEQ ID NO: 2; V at a residue corresponding to position 41 in SEQ ID NO: 2; I at a residue corresponding to position 45 in SEQ ID NO: 2; R, L, I, V at a residue corresponding to position 50 in SEQ ID NO: 2; F, M, L at a residue corresponding to position 58 in SEQ ID NO: 2; D at a residue corresponding to position 62 in SEQ ID NO: 2; D at a residue corresponding to position 63 in SEQ ID NO: 2; K at a residue corresponding to position 67 in SEQ ID NO: 2; F, L, Y at a residue corresponding to position 69 in SEQ ID NO: 2; A, R at a residue corresponding to position 72 in SEQ ID NO: 2; D, T, A, R at a residue corresponding to position 76 in SEQ ID NO: 2; R at a residue corresponding to position 77 in SEQ ID NO: 2; C at a residue corresponding to position 81 in SEQ ID NO: 2; Q at a residue corresponding to position 86 in SEQ ID NO: 2; R at a residue corresponding to position 87 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; M at a residue corresponding to position 98 in SEQ ID NO: 2; A at a residue corresponding to position 104 in SEQ ID NO: 2; V at a residue corresponding to position 108 in SEQ ID NO: 2; C at a residue corresponding to position 113 in SEQ ID NO: 2; I at a residue corresponding to position 116 in SEQ ID NO: 2; Y, V, F, L at a residue corresponding to position 120 in SEQ ID NO: 2; I at a residue corresponding to position 123 in SEQ ID NO: 2; S at a residue corresponding to position 124 in SEQ ID NO: 2; P, T at a residue corresponding to position 125 in SEQ ID NO: 2; R, A, N at a residue corresponding to position 126 in SEQ ID NO: 2; L at a residue corresponding to position 127 in SEQ ID NO: 2; M at a residue corresponding to position 130 in SEQ ID NO: 2; E at a residue corresponding to position 136 in SEQ ID NO: 2; M, I at a residue corresponding to position 140 in SEQ ID NO: 2; M at a residue corresponding to position 141 in SEQ ID NO: 2; Q, N at a residue corresponding to position 142 in SEQ ID NO: 2; R at a residue corresponding to position 145 in SEQ ID NO: 2; S at a residue corresponding to position 147 in SEQ ID NO: 2; V at a residue corresponding to position 148 in SEQ ID NO: 2; V at a residue corresponding to position 149 in SEQ ID NO: 2; C at a residue corresponding to position 151 in SEQ ID NO: 2; E at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 190 in SEQ ID NO: 2; E, A at a residue corresponding to position 191 in SEQ ID NO: 2; A at a residue corresponding to position 193 in SEQ ID NO: 2; S at a residue corresponding to position 195 in SEQ ID NO: 2; L, A at a residue corresponding to position 201 in SEQ ID NO: 2; T at a residue corresponding to position 205 in SEQ ID NO: 2; I at a residue corresponding to position 209 in SEQ ID NO: 2; Y at a residue corresponding to position 214 in SEQ ID NO: 2; T at a residue corresponding to position 219 in SEQ ID NO: 2; G at a residue corresponding to position 220 in SEQ ID NO: 2; K at a residue corresponding to position 221 in SEQ ID NO: 2; S at a residue corresponding to position 228 in SEQ ID NO: 2; H, K at a residue corresponding to position 230 in SEQ ID NO: 2; L at a residue corresponding to position 232 in SEQ ID NO: 2; S at a residue corresponding to position 234 in SEQ ID NO: 2; A at a residue corresponding to position 237 in SEQ ID NO: 2; M at a residue corresponding to position 238 in SEQ ID NO: 2; V at a residue corresponding to position 239 in SEQ ID NO: 2; Q, E at a residue corresponding to position 241 in SEQ ID NO: 2; C, V, T at a residue corresponding to position 242 in SEQ ID NO: 2; W, F, M at a residue corresponding to position 243 in SEQ ID NO: 2; P, Q at a residue corresponding to position 244 in SEQ ID NO: 2; I at a residue corresponding to position 246 in SEQ ID NO: 2; R at a residue corresponding to position 247 in SEQ ID NO: 2; L at a residue corresponding to position 253 in SEQ ID NO: 2; M at a residue corresponding to position 254 in SEQ ID NO: 2; S, H at a residue corresponding to position 260 in SEQ ID NO: 2; Y at a residue corresponding to position 263 in SEQ ID NO: 2; N at a residue corresponding to position 266 in SEQ ID NO: 2; M, F at a residue corresponding to position 267 in SEQ ID NO: 2; A at a residue corresponding to position 272 in SEQ ID NO: 2; I at a residue corresponding to position 273 in SEQ ID NO: 2; C at a residue corresponding to position 274 in SEQ ID NO: 2; T, A at a residue corresponding to position 278 in SEQ ID NO: 2; M at a residue corresponding to position 279 in SEQ ID NO: 2; V at a residue corresponding to position 281 in SEQ ID NO: 2; E at a residue corresponding to position 283 in SEQ ID NO: 2; M, I, L, V at a residue corresponding to position 291 in SEQ ID NO: 2; H at a residue corresponding to position 292 in SEQ ID NO: 2; V, I, M at a residue corresponding to position 295 in SEQ ID NO: 2; M, V at a residue corresponding to position 298 in SEQ ID NO: 2; Q at a residue corresponding to position 300 in SEQ ID NO: 2; Q at a residue corresponding to position 302 in SEQ ID NO: 2; A, P at a residue corresponding to position 303 in SEQ ID NO: 2; S at a residue corresponding to position 304 in SEQ ID NO: 2; V at a residue corresponding to position 305 in SEQ ID NO: 2; M at a residue corresponding to position 307 in SEQ ID NO: 2; T, A at a residue corresponding to position 311 in SEQ ID NO: 2; F at a residue corresponding to position 313 in SEQ ID NO: 2; Q, H at a residue corresponding to position 314 in SEQ ID NO: 2; A, K at a residue corresponding to position 315 in SEQ ID NO: 2; D at a residue corresponding to position 318 in SEQ ID NO: 2; R, Q at a residue corresponding to position 321 in SEQ ID NO: 2; A, K, H, Q at a residue corresponding to position 322 in SEQ ID NO: 2; F at a residue corresponding to position 330 in SEQ ID NO: 2; S at a residue corresponding to position 333 in SEQ ID NO: 2; V, I at a residue corresponding to position 334 in SEQ ID NO: 2; V, T, M at a residue corresponding to position 336 in SEQ ID NO: 2; L, V at a residue corresponding to position 337 in SEQ ID NO: 2; L, M at a residue corresponding to position 338 in SEQ ID NO: 2; G at a residue corresponding to position 340 in SEQ ID NO: 2; I at a residue corresponding to position 344 in SEQ ID NO: 2; I, L at a residue corresponding to position 348 in SEQ ID NO: 2; Y at a residue corresponding to position 349 in SEQ ID NO: 2; R, Q at a residue corresponding to position 351 in SEQ ID NO: 2; R at a residue corresponding to position 353 in SEQ ID NO: 2; K at a residue corresponding to position 354 in SEQ ID NO: 2; A at a residue corresponding to position 355 in SEQ ID NO: 2; F, I at a residue corresponding to position 357 in SEQ ID NO: 2; Q at a residue corresponding to position 358 in SEQ ID NO: 2; Q at a residue corresponding to position 359 in SEQ ID NO: 2; C, H at a residue corresponding to position 362 in SEQ ID NO: 2; V at a residue corresponding to position 363 in SEQ ID NO: 2; M at a residue corresponding to position 365 in SEQ ID NO: 2; V, I, L, M at a residue corresponding to position 367 in SEQ ID NO: 2; A at a residue corresponding to position 369 in SEQ ID NO: 2; G at a residue corresponding to position 376 in SEQ ID NO: 2; V, C at a residue corresponding to position 379 in SEQ ID NO: 2; T, C at a residue corresponding to position 382 in SEQ ID NO: 2; Y at a residue corresponding to position 383 in SEQ ID NO: 2; V, T at a residue corresponding to position 386 in SEQ ID NO: 2; R, H, N at a residue corresponding to position 387 in SEQ ID NO: 2; K at a residue corresponding to position 388 in SEQ ID NO: 2; A, P at a residue corresponding to position 389 in SEQ ID NO: 2; C, I, T, V at a residue corresponding to position 399 in SEQ ID NO: 2; Q at a residue corresponding to position 400 in SEQ ID NO: 2; L, I at a residue corresponding to position 401 in SEQ ID NO: 2; V at a residue corresponding to position 404 in SEQ ID NO: 2; P at a residue corresponding to position 407 in SEQ ID NO: 2; G at a residue corresponding to position 409 in SEQ ID NO: 2; V at a residue corresponding to position 420 in SEQ ID NO: 2; F at a residue corresponding to position 421 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; V at a residue corresponding to position 434 in SEQ ID NO: 2; Y, R, K, C, H at a residue corresponding to position 442 in SEQ ID NO: 2; L, V, M, I at a residue corresponding to position 446 in SEQ ID NO: 2; M at a residue corresponding to position 450 in SEQ ID NO: 2; P at a residue corresponding to position 452 in SEQ ID NO: 2; G at a residue corresponding to position 453 in SEQ ID NO: 2; N, D at a residue corresponding to position 456 in SEQ ID NO: 2; F, I at a residue corresponding to position 460 in SEQ ID NO: 2; Y at a residue corresponding to position 461 in SEQ ID NO: 2; L at a residue corresponding to position 465 in SEQ ID NO: 2; A at a residue corresponding to position 466 in SEQ ID NO: 2; N, S at a residue corresponding to position 473 in SEQ ID NO: 2; Y, F, V, L, I at a residue corresponding to position 476 in SEQ ID NO: 2; V, P at a residue corresponding to position 479 in SEQ ID NO: 2; S, A at a residue corresponding to position 480 in SEQ ID NO: 2; P, E at a residue corresponding to position 481 in SEQ ID NO: 2; L, E, R, A at a residue corresponding to position 485 in SEQ ID NO: 2; P at a residue corresponding to position 492 in SEQ ID NO: 2; V at a residue corresponding to position 507 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P at a residue corresponding to position 521 in SEQ ID NO: 2; M, L at a residue corresponding to position 526 in SEQ ID NO: 2; L at a residue corresponding to position 527 in SEQ ID NO: 2; R, H at a residue corresponding to position 532 in SEQ ID NO: 2; Q at a residue corresponding to position 540 in SEQ ID NO: 2; Y at a residue corresponding to position 542 in SEQ ID NO: 2; E at a residue corresponding to position 554 in SEQ ID NO: 2; S, T at a residue corresponding to position 564 in SEQ ID NO: 2; T, M, P, V, I at a residue corresponding to position 565 in SEQ ID NO: 2; A, S, Q, N at a residue corresponding to position 567 in SEQ ID NO: 2; G, D at a residue corresponding to position 568 in SEQ ID NO: 2; V at a residue corresponding to position 570 in SEQ ID NO: 2; P at a residue corresponding to position 572 in SEQ ID NO: 2; T at a residue corresponding to position 575 in SEQ ID NO: 2; N at a residue corresponding to position 584 in SEQ ID NO: 2; A at a residue corresponding to position 587 in SEQ ID NO: 2; E at a residue corresponding to position 595 in SEQ ID NO: 2; K at a residue corresponding to position 596 in SEQ ID NO: 2; or any combination thereof.

In some embodiments, provided herein is microbial organism (e.g., host microbial organism) that has a recombinant nucleic acid encoding an engineered ACL having one or more amino acid alterations comprising: D5R, D5E, A6L, S7K, S7T, G8P, E10K, E10D, E10P, E10N, E10R, L12A, L12I, L12V, L12M, P13C, H14P, H14A, H14Q, D15E, D15L, D15S, D15R, V16C, V16L, V161, E19D, E19R, S20H, S20K, S20R, S20D, P22D, P22E, S25N, L28M, V37C, P39R, N40T, T41M, T41L, T41V, T41I, A43S, W44M, L45I, L45V, W48P, K50I, K50F, K50R, K50V, K50L, E51R, P53L, A57F, I58V, I58L, I58M, I58F, A59V, R61P, R61E, A62D, G63D, W66G, R67K, H69F, H69Y, H69L, Y71F, Y71W, N72A, N72R, L74F, L75D, N76T, N76D, N76A, N76R, M77R, V78A, N80K, V81C, G83S, G83H, G83Q, G83N, A84Y, A84H, L85F, L86A, L86Q, A87R, R88L, A92K, D93G, S94R, P95R, V97A, V98M, M99L, S100C, G101P, G103R, V104L, V104W, V104A, H106W, H106F, A107V, A107P, A107I, A107L, A107W, A107F, I108V, L109V, S110M, S110F, S110Y, S110A, SHOD, A113T, A113C, A113V, Q114M, Q114W, Q114L, Q114G, Q114A, Y115A, V116I, P119V, P119T, P119I, I120M, I120F, I120V, I120A, I120L, I120W, I120Y, V121T, P122A, L123M, L123V, L123I, A124Y, A124S, A124N, E125P, E125T, E125W, Q126M, Q126N, Q126R, Q126A, Y127F, Y127W, Y127L, S128R, I130M, P131E, R136E, R136Q, R136K, L137M, V140M, V140C, V140I, L141M, E142N, E142Q, K145G, K145R, K145D, A147S, M148L, M148V, M148A, A149V, A149L, F150V, V151C, Y157L, S159R, S159E, L161I, L163V, P164A, Q165R, E167R, G168H, V169I, E170I, E170V, E170L, A173V, K175R, T176D, D177E, V181E, V183I, A185S, F186Y, A187D, L189V, L190V, G191A, G191E, H192T, G193A, Q194S, G195S, G196P, H201A, H201P, H201L, G205T, T208D, L209I, L209T, L209P, L209V, K211L, K211T, K211C, K211M, K211V, K211A, I212L, I212M, L213I, L213C, L213V, L213Q, L213M, F214Y, T215S, S216G, S218T, S219T, S220G, D221T, D221M, D221L, D221R, D221K, D221H, P222S, G224A, V225A, L226M, L226I, L226V, T227L, T227D, T227H, T227I, T228S, R230H, R230K, M231N, M232L, M232I, M232V, C233L, C233V, C233M, C233W, C233A, C233I, A234S, A234W, N235Q, N235S, Q236A, Q236C, Q236I, Q236M, Q236V, T237A, T237M, T237Q, T237R, T237E, Q238M, Q238S, Q238W, Q238N, Q238H, I239F, I239M, I239V, I239C, I239L, S240A, S240R, D241A, D241H, D241E, D241Q, A242V, A242C, A242T, A242W, L243I, L243F, L243M, L243W, L243V, R244E, R244P, R244Q, R244H, R244G, F245M, F245L, F245H, F245W, F245Y, L246I, L246T, L246M, K247R, K247P, K247E, P251E, R252H, R252V, I253Y, I253M, I253F, I253L, I253V, L254I, L254F, L254V, L254M, D255A, D255T, D255N, D255L, D255I, D255G, D255H, D255C, D255V, D255S, W256F, W256V, W256I, W256C, W256Y, L257M, W259L, W259F, N260H, N260S, N260Y, N260F, V262C, V262T, V262A, V262I, V262M, F263A, F263Y, F263H, G264A, G265M, G265L, G265W, G265F, G265C, S266N, S266T, S266M, S266V, H267I, H267G, H267M, H267W, H267F, H267L, H267V, H267C, H267A, N268M, N268A, N268C, N268G, N268V, F269L, F269M, F269I, F269V, N270M, N270H, N270Y, N270T, M271A, M271P, M271C, M271T, M271L, M272C, M272V, M272P, M272A, M272T, L273M, L273V, L273F, L273I, A274Y, A274R, A274V, A274M, A274C, A274H, A274W, N275G, N275C, N275S, N275H, G277A, G277C, S278C, S278T, S278A, S278H, L279V, L279M, L279I, I281F, I281L, I281V, I281M, D282P, D282M, D282E, D282A, D282H, N283E, N283D, N283P, N283R, G284F, G284R, G284W, K285R, K285F, K285W, K285D, P286D, P286F, T287P, T287A, T287V, G290A, F291M, F291V, F291L, F291P, F291I, E292H, T294I, T294L, T294A, L295W, L295M, L295V, L295I, R296D, R296Q, I298M, I298V, I298L, K299A, E300Q, P302Q, G303V, G303I, G303A, G303P, G303C, T304S, L305W, L305Y, L305V, L305I, L305F, L305H, L305M, A306V, A306Y, A306I, A306M, A306L, A306F, F307P, F307W, F307Y, F307H, F307M, N308T, N308A, N308S, N308L, N308G, N308V, N308M, V311A, V311R, V311P, V311T, G312I, G312M, G312V, G312F, Y313L, Y313F, Y313W, A314Q, A314E, A314H, M315R, M315A, M315K, M315L, L316W, L316M, L316I, L316F, N318H, N318D, E321R, E321Q, R322Q, R322K, R322A, R322H, Y330L, Y330F, F331G, E333S, L334I, L334M, L334V, D335R, D335K, L336T, L336I, L336M, L336Y, L336V, L336F, L336W, I337L, I337M, I337F, I337A, I337V, F338W, F338L, F338M, F338V, Y339F, Y339T, Y339S, Y339C, A340G, G341A, G341S, A342S, S343P, S343A, L344M, L344I, L344V, L344C, P345S, Q346V, Q346A, Q346P, Q346E, D347E, V348T, V348I, V348L, W349M, W349F, W349L, W349A, W349I, W349Y, S351F, S351R, S351Q, S351W, L352I, L352M, L352Y, E353R, R354K, F355A, F355R, M357W, M357G, M357V, M357L, M357P, M357I, M357F, M357A, E358Q, E359H, E359Q, G361F, S362H, S362L, S362C, L363D, L363V, L363I, L363G, L365M, L365V, L365I, M366I, M366V, M366L, M366F, A367T, A367C, A367M, A367I, A367V, A367L, S368N, S368Q, S368E, S368T, S369G, S369A, W370Y, W370F, M372L, M372Q, T373S, T375A, T375C, A376T, A376S, A376C, A376G, P377G, P377M, A378C, A378T, A378V, T379C, T379V, T379I, T379L, T379A, I380F, I380C, I380H, I380S, I380M, I380T, I380V, M381N, M381F, M381L, M381C, M381V, V382L, V382N, V382C, V382T, V382P, H383D, H383N, H383R, H383P, H383Y, E384P, E384N, E384D, E384W, P385G, P385D, I386D, I386V, I386L, I386T, G387D, G387R, G387N, G387H, G387E, R388K, R388H, S389A, S389P, S389L, V391S, V391T, I392V, I392C, V394R, V394K, V394I, V394Q, V394L, P395A, L396F, L396I, L396M, L396V, G398D, G398H, G398N, A399M, A399V, A399T, A399C, A399I, E400D, E400Q, V401M, V401I, V401C, V401L, V401A, K402R, L404V, P405D, D406E, D407P, M409Q, M409E, M409G, C411D, C411S, C411V, C411G, C411N, L413I, L413V, R414W, R414L, R414V, R414C, R414Q, V415I, K416R, G417S, P418D, N419H, N419S, N419Q, N419M, I420V, M421T, M421F, F425H, F425L, K430A, A432K, E433R, E433D, A434T, A434V, F435I, F435M, F435L, L441F, L441M, L441Y, V442K, V442H, V442Y, V442R, V442C, T443S, A446M, A446I, A446L, A446V, V447A, V447G, F449I, V450D, V450M, D451E, D452P, A453G, P455E, P455G, P455R, S456D, S456E, S456Q, S456N, G458N, G458F, G458Y, G458W, L459Y, V460Y, V460F, V460I, V460R, V460W, F461I, F461L, F461Y, D462I, D462T, D462V, G463D, R464D, V465L, S466D, S466K, S466A, E467K, D468M, F469L, K470V, L471T, L471V, T473S, T473N, G474E, W476N, W476Y, W476L, W476F, W476V, W476I, Q478T, Q478L, A479P, A479V, G480L, G480I, G480S, G480P, G480A, G480V, V481P, V481E, R483E, D485A, D485L, D485E, D485R, L490C, G492P, L493F, D496H, V497A, V498C, H502P, R504H, I507Y, I507V, I507W, G508V, F510A, I511V, I511L, F512I, P513V, L514N, P515D, P515S, G516A, V518I, S519T, T521P, T521W, V526L, V526M, I527L, S532R, S532H, I535L, R540Q, M542Y, M542W, A546N, R552Q, N554E, I558F, S560D, E561D, P563F, P563L, P563W, Q564D, Q564K, Q564S, Q564L, Q564T, Q564R, L565M, L565I, L565V, L565P, L565T, K566A, K566E, K566D, K566P, D567K, D567Q, D567T, D567N, D567S, D567A, H568G, H568N, H568D, E569M, E569K, I570V, I570L, D572R, D572P, K573T, K573S, G574M, G574L, S575A, S575Y, S575K, S575T, N577Y, N577A, N577L, I578Q, I578A, T583D, R584N, R585H, R585L, D587A, L588Q, R591Q, R591E, Y593A, N595E, E596K, P598D, A599S, I601L or any combination thereof.

In some embodiments, provided herein is microbial organism (e.g., host microbial organism) that has a recombinant nucleic acid encoding an engineered ACL having one or more amino acid alterations comprising: D5E, D5R, A6L, S7K, S7T, G8P, E10D, E10N, E10R, E10P, E10K, L12A, L12V, L12M, L12I, P13C, H14Q, H14P, H14A, D15S, D15E, V16I, V16C, E19D, E19R, S20H, S20K, S20R, S20D, P22E, P22D, S25N, L28M, V37C, P39R, N40T, T41V, T41I, T41M, A43S, L45I, L45V, K50R, K50L, K50I, K50V, K50F, E51R, P53L, A57F, I58F, I58M, I58L, I58V, A59V, R61P, A62D, G63D, R67K, H69F, H69L, H69Y, Y71W, Y71F, N72A, N72R, L74F, L75D, N76D, N76T, N76A, N76R, M77R, V78A, N80K, V81C, G83S, G83N, A84Y, A84H, L85F, L86Q, L86A, A87R, R88L, A92K, D93G, S94R, P95R, V97A, V98M, M99L, S100C, G101P, G103R, V104A, V104L, V104W, H106F, 1108V, L109V, S110A, S110M, A113C, V116I, P119T, P119V, P119I, I120Y, I120V, I120F, I120L, I120M, I120A, V121T, L123I, L123M, L123V, A124S, A124N, A124Y, E125P, E125T, E125W, Q126R, Q126A, Q126N, Q126M, Y127L, Y127W, Y127F, S128R, I130M, P131E, R136E, R136K, R136Q, L137M, V140M, V140I, V140C, L141M, E142Q, E142N, K145R, K145D, K145G, A147S, M148V, M148L, M148A, A149V, F150V, V151C, S159E, S159R, L161I, L163V, P164A, Q165R, E167R, G168H, V169I, E170I, E170L, E170V, A173V, K175R, T176D, D177E, V181E, V183I, A185S, F186Y, A187D, L189V, L190V, G191E, G191A, H192T, G193A, Q194S, G195S, G196P, H201L, H201A, G205T, T208D, L209I, L209T, L209V, K211L, I212L, I212M, L213I, L213M, L213V, F214Y, T215S, S218T, S219T, S220G, D221K, D221L, D221T, D221H, D221R, D221M, P222S, G224A, L226M, L226V, L226I, T227I, T228S, R230H, R230K, M232L, C233A, A234S, N235Q, Q236M, T237A, T237M, T237Q, Q238M, Q238H, I239V, I239L, I239M, S240A, D241Q, D241E, D241H, D241A, A242C, A242V, A242T, L243W, L243F, L243M, L243I, L243V, R244P, R244Q, R244G, R244H, R244E, F245L, F245M, F245W, L246I, K247R, K247P, R252V, R252H, I253L, I253V, I253M, L254M, L254I, L254V, D255C, D255V, D255T, D255N, W256Y, W256V, W256F, N260S, N260H, N260F, N260Y, V262T, V262C, V262A, F263Y, G264A, G265M, S266N, S266M, H267M, H267F, H267L, H267W, H267G, N268M, N268A, N270M, M271L, M271C, M271A, M272A, M272V, M272T, M272P, M272C, L273I, L273V, L273M, L273F, A274C, A274M, A274H, A274R, A274Y, N275H, N275C, G277A, S278T, S278A, S278C, L279M, L279I, L279V, I281V, I281L, N283E, N283D, N283P, N283R, K285R, K285F, K285D, P286D, P286F, T287A, T287P, T287V, G290A, F291M, F291I, F291L, F291V, E292H, T294A, L295V, L295I, L295M, R296Q, R296D, I298M, I298V, I298L, K299A, E300Q, P302Q, G303A, G303P, G303C, G303V, T304S, L305V, L305M, L305I, A306I, A306V, A306Y, F307M, F307W, F307P, N308G, V311T, V311A, V311R, G312F, G312V, G312M, Y313F, Y313W, A314Q, A314H, A314E, M315A, M315K, M315L, M315R, L316I, L316M, N318D, N318H, E321R, E321Q, R322A, R322K, R322H, R322Q, Y330F, F331G, E333S, L334V, L334I, L334M, D335K, L336V, L336T, L336M, L336Y, L336W, L336I, L336F, I337L, I337V, I337M, F338L, F338M, F338V, F338W, Y339T, Y339S, Y339F, A340G, A342S, S343P, S343A, L344I, L344C, L344M, L344V, P345S, Q346E, Q346V, Q346A, Q346P, D347E, V348I, V348L, V348T, W349Y, W349F, W349L, S351R, S351Q, S351W, L352I, L352M, E353R, R354K, F355A, M357F, M357I, M357V, M357A, M357G, M357L, M357W, E358Q, E359Q, S362C, S362H, S362L, L363V, L363I, L363G, L365M, L365I, L365V, M366I, M366L, M366F, M366V, A367V, A367I, A367L, A367M, A367T, A367C, S368N, S369A, S369G, W370Y, T375C, T375A, A376G, A376S, P377G, A378T, T379V, T379C, T379L, T379A, T379I, 1380S, I380V, I380H, I380T, M381L, M381C, V382T, V382C, V382N, V382L, V382P, H383Y, H383R, E384N, E384W, E384D, P385G, I386V, I386T, I386L, G387R, G387H, G387N, G387D, G387E, R388K, R388H, S389A, S389P, S389L, V391S, V391T, I392V, V394I, V394K, L396V, G398H, A399C, A399I, A399T, A399V, E400Q, E400D, V401L, V401I, V401C, V401A, V401M, L404V, D406E, D407P, M409G, M409Q, M409E, C411S, R414L, R414C, K416R, N419Q, N419H, N419M, N419S, I420V, M421F, M421T, F425L, F425H, K430A, A432K, E433R, E433D, A434V, A434T, F435M, L441F, L441M, V442Y, V442R, V442K, V442C, V442H, T443S, A446L, A446V, A446M, A446I, V447A, V450M, V450D, D451E, D452P, A453G, S456N, S456D, V460F, V460I, V460R, V460W, V460Y, F461Y, F461L, D462V, D462I, G463D, V465L, S466A, S466D, S466K, E467K, D468M, F469L, K470V, L471T, L471V, T473N, T473S, W476Y, W476F, W476V, W476L, W476I, W476N, Q478T, A479V, A479P, G480S, G480A, G480P, G480L, V481P, V481E, D485L, D485E, D485R, D485A, L490C, G492P, V498C, I507V, G508V, F510A, I511V, I511L, F512I, L514N, P515D, P515S, G516A, V518I, S519T, T521P, T521W, V526M, V526L, I527L, S532R, S532H, I535L, R540Q, M542Y, M542W, A546N, R552Q, N554E, S560D, E561D, P563L, P563F, Q564S, Q564T, Q564L, Q564D, Q564R, Q564K, L565T, L565M, L565P, L565V, L565I, K566D, K566A, K566E, K566P, D567A, D567S, D567Q, D567N, D567T, D567K, H568G, H568D, H568N, E569M, E569K, I570V, I570L, D572P, K573S, K573T, G574M, G574L, S575T, S575Y, N577A, N577L, I578A, I578Q, T583D, R584N, R585H, D587A, L588Q, R591Q, R591E, Y593A, N595E, E596K, P598D, A599S, or I601L

In some embodiments, provided herein is microbial organism (e.g., host microbial organism) that has a recombinant nucleic acid encoding an engineered ACL having one or more amino acid alterations comprising: D5E, D5R, A6L, S7K, S7T, G8P, E10D, E10N, E10R, E10P, L12A, L12V, L12M, H14Q, H14P, D15S, D15E, V16I, E19D, E19R, S20H, S20K, S20R, S20D, P22E, P22D, L28M, P39R, T41V, A43S, L45I, K50R, K50L, K50I, K50V, K50F, A57F, I58F, I58M, I58L, I58V, R61P, A62D, G63D, R67K, H69F, H69L, H69Y, Y71W, Y71F, N72A, N72R, N76D, N76T, N76A, N76R, M77R, V78A, V81C, A84Y, L85F, L86Q, A87R, A92K, D93G, S94R, P95R, V98M, V104A, I108V, L109V, A113C, V116I, P119T, I120Y, I120V, I120F, I120L, I120M, I120A, L123I, A124S, A124N, E125P, E125T, E125W, Q126R, Q126A, Q126N, Y127L, Y127W, I130M, P131E, R136E, R136K, V140M, V140I, V140C, L141M, E142Q, E142N, K145R, A147S, M148V, A149V, V151C, S159E, S159R, L161I, L163V, P164A, E167R, G168H, V169I, E170I, E170L, E170V, A173V, K175R, T176D, D177E, V181E, V183I, A185S, F186Y, A187D, L190V, G191E, G191A, G193A, Q194S, G195S, H201L, H201A, G205T, L209I, F214Y, S218T, S219T, S220G, D221K, D221L, D221T, D221H, L226M, L226V, T228S, R230H, R230K, M232L, A234S, T237A, Q238M, I239V, D241Q, D241E, A242C, A242V, A242T, L243W, L243F, L243M, R244P, R244Q, L246I, K247R, I253L, I253V, L254M, L254I, L254V, N260S, N260H, V262T, F263Y, S266N, H267M, H267F, M272A, L273I, L273V, L273M, L273F, A274C, S278T, S278A, L279M, I281V, N283E, K285R, G290A, F291M, F291I, F291L, F291V, E292H, L295V, L295I, L295M, R296Q, I298M, I298V, E300Q, P302Q, G303A, G303P, T304S, L305V, L305M, L305I, F307M, V311T, V311A, Y313F, A314Q, A314H, M315A, M315K, M315L, M315R, L316I, N318D, N318H, E321R, E321Q, R322A, R322K, R322H, R322Q, Y330F, E333S, L334V, L334I, L336V, L336T, L336M, L336Y, I337L, I337V, F338L, F338M, F338V, Y339T, A340G, L344I, L344C, L344M, P345S, Q346E, D347E, V348I, V348L, V348T, W349Y, W349F, S351R, S351Q, L352I, E353R, R354K, F355A, M357F, M357I, M357V, M357A, M357G, M357L, M357W, E358Q, E359Q, S362C, S362H, L363V, L363I, L365M, A367V, A367I, A367L, A367M, S369A, T375C, A376G, A376S, T379V, T379C, T379L, V382T, V382C, H383Y, E384N, I386V, I386T, G387R, G387H, G387N, G387D, R388K, R388H, S389A, S389P, S389L, A399C, A399I, A399T, A399V, E400Q, V401L, V401I, V401C, L404V, D407P, M409G, K416R, N419Q, I420V, M421F, K430A, A432K, E433R, E433D, A434V, L441F, V442Y, V442R, V442K, V442C, V442H, A446L, A446V, A446M, A446I, V450M, D452P, A453G, S456N, S456D, V460F, V460I, V460R, F461Y, V465L, S466A, S466D, L471T, T473N, T473S, W476Y, W476F, W476V, W476L, W476I, A479V, A479P, G480S, G480A, G480P, V481P, V481E, D485L, D485E, D485R, D485A, L490C, G492P, I507V, I511V, L514N, P515D, P515S, G516A, V518I, S519T, T521P, T521W, V526M, V526L, I527L, S532R, S532H, R540Q, M542Y, A546N, N554E, E561D, Q564S, Q564T, L565T, L565M, L565P, L565V, L565I, K566D, D567A, D567S, D567Q, D567N, H568G, H568D, H568N, I570V, D572P, G574M, S575T, N577A, R584N, D587A, N595E, E596K, or A599S

In some embodiments, provided herein is microbial organism (e.g., host microbial organism) that has a recombinant nucleic acid encoding an engineered ACL having one or more amino acid alterations comprising: D5E, D5R, A6L, S7K, S7T, G8P, E10D, E10N, L12A, H14Q, H14P, D15S, D15E, E19D, S20H, S20K, S20R, P22E, T41V, L45I, K50R, K50L, K50I, K50V, I58F, I58M, I58L, A62D, G63D, R67K, H69F, H69L, H69Y, N72A, N72R, N76D, N76T, N76A, N76R, M77R, V81C, L86Q, A87R, A92K, D93G, S94R, P95R, V98M, V104A, I108V, A113C, V116I, I120Y, I120V, I120F, I120L, L123I, A124S, E125P, E125T, Q126R, Q126A, Q126N, Y127L, I130M, R136E, V140M, V140I, L141M, E142Q, E142N, K145R, A147S, M148V, A149V, V151C, S159E, L161I, L163V, P164A, E167R, G168H, V169I, K175R, T176D, D177E, A185S, F186Y, A187D, L190V, G191E, G191A, G193A, G195S, H201L, H201A, G205T, L209I, F214Y, S219T, S220G, D221K, T228S, R230H, R230K, M232L, A234S, T237A, Q238M, I239V, D241Q, D241E, A242C, A242V, A242T, L243W, L243F, L243M, R244P, R244Q, L246I, K247R, I253L, L254M, N260S, N260H, F263Y, S266N, H267M, H267F, M272A, L273I, A274C, S278T, S278A, L279M, I281V, N283E, F291M, F291I, F291L, F291V, E292H, L295V, L295I, L295M, I298M, I298V, E300Q, P302Q, G303A, G303P, T304S, L305V, F307M, V311T, V311A, Y313F, A314Q, A314H, M315A, M315K, N318D, E321R, E321Q, R322A, R322K, R322H, R322Q, Y330F, E333S, L334V, L334I, L336V, L336T, L336M, I337L, I337V, F338L, F338M, A340G, L344I, V348I, V348L, W349Y, S351R, S351Q, E353R, R354K, F355A, M357F, M357I, E358Q, E359Q, S362C, S362H, L363V, L365M, A367V, A367I, A367L, A367M, S369A, A376G, T379V, T379C, V382T, V382C, H383Y, I386V, I386T, G387R, G387H, G387N, R388K, S389A, S389P, A399C, A399I, A399T, A399V, E400Q, V401L, V401I, L404V, D407P, M409G, I420V, M421F, K430A, A432K, E433R, E433D, A434V, V442Y, V442R, V442K, V442C, V442H, A446L, A446V, A446M, A446I, V450M, D452P, A453G, S456N, S456D, V460F, V460I, F461Y, V465L, S466A, T473N, T473S, W476Y, W476F, W476V, W476L, W476I, A479V, A479P, G480S, G480A, V481P, V481E, D485L, D485E, D485R, D485A, G492P, I507V, L514N, P515D, P515S, G516A, V518I, S519T, T521P, V526M, V526L, I527L, S532R, S532H, R540Q, M542Y, N554E, Q564S, Q564T, L565T, L565M, L565P, L565V, L565I, D567A, D567S, D567Q, D567N, H568G, H568D, I570V, D572P, S575T, R584N, D587A, N595E, or E596K. In some embodiments, provided herein is microbial organism (e.g., host microbial organism) that has a recombinant nucleic acid encoding an engineered ACL provided herein has an amino acid sequence that is a variant of SEQ ID NO: 2 that includes one or more alterations as described in TABLE 9, wherein the portion, other than the one or more alterations described in TABLE 9, of the engineered ACL has at least 65%, 70%, 75%, 80%, 85%, 90%, 95%, 98% or 99% sequence identity, or is identical, to an amino acid sequence referenced as SEQ ID NO: 2. Accordingly, in some embodiments, provided herein is microbial organism (e.g., host microbial organism) that has a recombinant nucleic acid encoding an engineered ACL that has an amino acid sequence that includes one or more alterations as described in TABLE 9 and the portion, other than the alteration described in TABLE 9, of the engineered ACL has at least 65% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and the portion, other than the alteration described in TABLE 9, of the engineered ACL has at least 70% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and the portion, other than the alteration described in TABLE 9, of the engineered ACL has at least 75% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and the portion, other than the alteration described in TABLE 9, of the engineered ACL has at least 80% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and the portion, other than the alteration described in TABLE 9, of the engineered ACL has at least 85% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and the portion, other than the alteration described in TABLE 9, of the engineered ACL has at least 90% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and the portion, other than the alteration described in TABLE 9, of the engineered ACL has at least 95% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and the portion, other than the alteration described in TABLE 9, of the engineered ACL has at least 98% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and the portion, other than the alteration described in TABLE 9, of the engineered ACL has at least 99% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and the portion, other than the alteration described in TABLE 9, of the engineered ACL is identical to SEQ ID NO: 2.

In some embodiments, provided herein is a microbial organism, in particular a non-naturally occurring microbial organism, that expresses an engineered ACL described herein, that is, an engineered ACL described herein. Thus, provided herein is a non-naturally occurring microbial organism having a recombinant nucleic acid encoding an engineered ACL described herein. Accordingly, in some embodiments, provided herein is microbial organism (e.g., host microbial organism) that has a recombinant nucleic acid encoding an engineered ACL that is a variant of a wild-type or parent ACL (SEQ ID NO: 1), such as an engineered ACL having one or more alterations at a position described in TABLE 8, and/or one or more, or a combination of alternations described in TABLE 9.

In some embodiments, provided herein is microbial organism (e.g., host microbial organism) that has a recombinant nucleic acid encoding an engineered ACL that is a variant of SEQ ID NO: 1 that includes one or more alterations at a position described in TABLE 8, and wherein the engineered ACL further includes one or more or a combination of alterations described in TABLE 9. Accordingly, in some embodiments, the microbial organism has a recombinant nucleic acid that encodes an engineered ACL having: M at a residue corresponding to position 4 in SEQ ID NO: 1; D, K, Q, S, T at a residue corresponding to position 7 in SEQ ID NO: 1; I, S at a residue corresponding to position 8 in SEQ ID NO: 1; A, K, S at a residue corresponding to position 9 in SEQ ID NO: 1; F, H, P, V, W, Y at a residue corresponding to position 10 in SEQ ID NO: 1; D, E, H, P at a residue corresponding to position 12 in SEQ ID NO: 1; C, M at a residue corresponding to position 13 in SEQ ID NO: 1; D, I, L, P at a residue corresponding to position 15 in SEQ ID NO: 1; E, H, N at a residue corresponding to position 16 in SEQ ID NO: 1; F, L, P, Q at a residue corresponding to position 19 in SEQ ID NO: 1; W at a residue corresponding to position 20 in SEQ ID NO: 1; S at a residue corresponding to position 22 in SEQ ID NO: 1; D at a residue corresponding to position 30 in SEQ ID NO: 1; A at a residue corresponding to position 31 in SEQ ID NO: 1; G, P at a residue corresponding to position 35 in SEQ ID NO: 1; H at a residue corresponding to position 36 in SEQ ID NO: 1; A at a residue corresponding to position 39 in SEQ ID NO: 1; C, F, L, M at a residue corresponding to position 40 in SEQ ID NO: 1; G at a residue corresponding to position 43 in SEQ ID NO: 1; D, H, S, T at a residue corresponding to position 44 in SEQ ID NO: 1; R, RTYTQKEL (SEQ ID NO: 15), RTYTQREL (SEQ ID NO: 16), RTYT (SEQ ID NO: 17) at a residue corresponding to position 47 in SEQ ID NO: 1; a deletion at a residue corresponding to position 48 in SEQ ID NO: 1; a deletion, Y at a residue corresponding to position 49 in SEQ ID NO: 1; a deletion, T at a residue corresponding to position 50 in SEQ ID NO: 1; a deletion, F, H, L, R, W, Y at a residue corresponding to position 51 in SEQ ID NO: 1; a deletion, K, R at a residue corresponding to position 52 in SEQ ID NO: 1; a deletion at a residue corresponding to position 53 in SEQ ID NO: 1; a deletion, L at a residue corresponding to position 54 in SEQ ID NO: 1; L at a residue corresponding to position 55 in SEQ ID NO: 1; R at a residue corresponding to position 60 in SEQ ID NO: 1; F, L at a residue corresponding to position 61 in SEQ ID NO: 1; A, G at a residue corresponding to position 64 in SEQ ID NO: 1; R at a residue corresponding to position 66 in SEQ ID NO: 1; A at a residue corresponding to position 67 in SEQ ID NO: 1; L at a residue corresponding to position 69 in SEQ ID NO: 1; G, T at a residue corresponding to position 72 in SEQ ID NO: 1; F, G, L, V at a residue corresponding to position 73 in SEQ ID NO: 1; K at a residue corresponding to position 74 in SEQ ID NO: 1; T at a residue corresponding to position 76 in SEQ ID NO: 1; V at a residue corresponding to position 78 in SEQ ID NO: 1; G at a residue corresponding to position 80 in SEQ ID NO: 1; PG at a residue corresponding to position 81 in SEQ ID NO: 1; A, E, G, N, R, T at a residue corresponding to position 82 in SEQ ID NO: 1; a deletion, K, P, R, S at a residue corresponding to position 83 in SEQ ID NO: 1; I at a residue corresponding to position 86 in SEQ ID NO: 1; I, V, VAV at a residue corresponding to position 87 in SEQ ID NO: 1; a deletion, A at a residue corresponding to position 88 in SEQ ID NO: 1; a deletion, I, L, V at a residue corresponding to position 89 in SEQ ID NO: 1; C, L, M, V, W at a residue corresponding to position 90 in SEQ ID NO: 1; A, L, M, S at a residue corresponding to position 91 in SEQ ID NO: 1; C, CIEAA (SEQ ID NO: 18), S, T, TIEAA (SEQ ID NO: 19) at a residue corresponding to position 94 in SEQ ID NO: 1; a deletion at a residue corresponding to position 95 in SEQ ID NO: 1; a deletion, E at a residue corresponding to position 96 in SEQ ID NO: 1; a deletion at a residue corresponding to position 97 in SEQ ID NO: 1; a deletion, A, L at a residue corresponding to position 98 in SEQ ID NO: 1; C, M at a residue corresponding to position 99 in SEQ ID NO: 1; S, M, L at a residue corresponding to position 100 in SEQ ID NO: 1; F, M at a residue corresponding to position 101 in SEQ ID NO: 1; H, F, L at a residue corresponding to position 102 in SEQ ID NO: 1; G at a residue corresponding to position 103 in SEQ ID NO: 1; V, L, T at a residue corresponding to position 104 in SEQ ID NO: 1; V, W, H, A, I at a residue corresponding to position 105 in SEQ ID NO: 1; S, Y, R, K at a residue corresponding to position 106 in SEQ ID NO: 1; A, C, V at a residue corresponding to position 107 in SEQ ID NO: 1; L, V, Q at a residue corresponding to position 109 in SEQ ID NO: 1; V, A, M, L, C, VTPAN (SEQ ID NO: 20) at a residue corresponding to position 111 in SEQ ID NO: 1; T, a deletion at a residue corresponding to position 112 in SEQ ID NO: 1; a deletion, L at a residue corresponding to position 113 in SEQ ID NO: 1; a deletion, A, L, V at a residue corresponding to position 114 in SEQ ID NO: 1; a deletion, N at a residue corresponding to position 115 in SEQ ID NO: 1; A, W, P at a residue corresponding to position 116 in SEQ ID NO: 1; S at a residue corresponding to position 117 in SEQ ID NO: 1; T at a residue corresponding to position 119 in SEQ ID NO: 1; R, D at a residue corresponding to position 121 in SEQ ID NO: 1; H at a residue corresponding to position 126 in SEQ ID NO: 1; M, L, MRHTG (SEQ ID NO: 21), MRITG (SEQ ID NO: 22) at a residue corresponding to position 127 in SEQ ID NO: 1; a deletion at a residue corresponding to position 128 in SEQ ID NO: 1; a deletion, I, H at a residue corresponding to position 129 in SEQ ID NO: 1; a deletion at a residue corresponding to position 130 in SEQ ID NO: 1; a deletion, G, D at a residue corresponding to position 131 in SEQ ID NO: 1; L at a residue corresponding to position 136 in SEQ ID NO: 1; T at a residue corresponding to position 137 in SEQ ID NO: 1; D, S at a residue corresponding to position 138 in SEQ ID NO: 1; E at a residue corresponding to position 139 in SEQ ID NO: 1; E, K, D at a residue corresponding to position 140 in SEQ ID NO: 1; T at a residue corresponding to position 144 in SEQ ID NO: 1; R at a residue corresponding to position 149 in SEQ ID NO: 1; I, L at a residue corresponding to position 150 in SEQ ID NO: 1; A, T, C, I at a residue corresponding to position 152 in SEQ ID NO: 1; G, S at a residue corresponding to position 153 in SEQ ID NO: 1; G at a residue corresponding to position 159 in SEQ ID NO: 1; S, H, R, SIDAF (SEQ ID NO: 23), RIESF (SEQ ID NO: 24), HFDSF (SEQ ID NO: 25), HFDAF (SEQ ID NO: 26), HIDSF (SEQ ID NO: 27) at a residue corresponding to position 162 in SEQ ID NO: 1; F, I, a deletion at a residue corresponding to position 163 in SEQ ID NO: 1; a deletion, D at a residue corresponding to position 164 in SEQ ID NO: 1; a deletion, S at a residue corresponding to position 165 in SEQ ID NO: 1; a deletion, F at a residue corresponding to position 166 in SEQ ID NO: 1; L at a residue corresponding to position 167 in SEQ ID NO: 1; G at a residue corresponding to position 170 in SEQ ID NO: 1; L, A, D at a residue corresponding to position 172 in SEQ ID NO: 1; Q at a residue corresponding to position 182 in SEQ ID NO: 1; E, G at a residue corresponding to position 189 in SEQ ID NO: 1; A at a residue corresponding to position 191 in SEQ ID NO: 1; D at a residue corresponding to position 194 in SEQ ID NO: 1; T, P, M at a residue corresponding to position 195 in SEQ ID NO: 1; T, I at a residue corresponding to position 196 in SEQ ID NO: 1; W, C at a residue corresponding to position 197 in SEQ ID NO: 1; Y, V, S, W, I, M, T at a residue corresponding to position 198 in SEQ ID NO: 1; M, L, W, P, F, C, A at a residue corresponding to position 199 in SEQ ID NO: 1; W at a residue corresponding to position 200 in SEQ ID NO: 1; F, T, N, P, Y, L at a residue corresponding to position 201 in SEQ ID NO: 1; T, H, A, Q, E, L, G at a residue corresponding to position 202 in SEQ ID NO: 1; T at a residue corresponding to position 205 in SEQ ID NO: 1; F at a residue corresponding to position 208 in SEQ ID NO: 1; P at a residue corresponding to position 209 in SEQ ID NO: 1; G at a residue corresponding to position 211 in SEQ ID NO: 1; A, C at a residue corresponding to position 212 in SEQ ID NO: 1; M, V, E at a residue corresponding to position 213 in SEQ ID NO: 1; V at a residue corresponding to position 214 in SEQ ID NO: 1; S at a residue corresponding to position 215 in SEQ ID NO: 1; Q, R, Y, H, K, G at a residue corresponding to position 217 in SEQ ID NO: 1; W, M, V at a residue corresponding to position 220 in SEQ ID NO: 1; T, A at a residue corresponding to position 221 in SEQ ID NO: 1; S, N at a residue corresponding to position 222 in SEQ ID NO: 1; A, N, I, L, H at a residue corresponding to position 223 in SEQ ID NO: 1; T, Q, H, I, N, M at a residue corresponding to position 224 in SEQ ID NO: 1; L, A, F, Q at a residue corresponding to position 225 in SEQ ID NO: 1; Y, C, T, W at a residue corresponding to position 226 in SEQ ID NO: 1; G, E, M, T, A, GKWSQD (SEQ ID NO: 28), GKWSQSY (SEQ ID NO: 29), GKWSQ (SEQ ID NO: 30), GKWSQDLK (SEQ ID NO: 31), GKWSQQLQ (SEQ ID NO: 32) at a residue corresponding to position 227 in SEQ ID NO: 1; a deletion at a residue corresponding to position 228 in SEQ ID NO: 1; a deletion, F, S, L, G, W, I, M, H at a residue corresponding to position 229 in SEQ ID NO: 1; a deletion, R, M, P, T, E, Q, A at a residue corresponding to position 230 in SEQ ID NO: 1; a deletion, Q, P, H, QDLQ (SEQ ID NO: 33) at a residue corresponding to position 231 in SEQ ID NO: 1; a deletion, W, A, Y, N, K, Q, D at a residue corresponding to position 232 in SEQ ID NO: 1; a deletion, W, R, M, Y, P at a residue corresponding to position 233 in SEQ ID NO: 1; a deletion, F, Y, T, K, R, Q, P, G, H, E at a residue corresponding to position 234 in SEQ ID NO: 1; H, S, R, K, M, Q, D, G at a residue corresponding to position 235 in SEQ ID NO: 1; P, N, H, T, D, DISAC (SEQ ID NO: 34), DVS at a residue corresponding to position 238 in SEQ ID NO: 1; V, F, R, a deletion at a residue corresponding to position 239 in SEQ ID NO: 1; a deletion, F, H, T, W, S, L, C, V at a residue corresponding to position 240 in SEQ ID NO: 1; a deletion, C, M, P, I at a residue corresponding to position 241 in SEQ ID NO: 1; a deletion, G, V, C at a residue corresponding to position 242 in SEQ ID NO: 1; L, C, M at a residue corresponding to position 243 in SEQ ID NO: 1; M, V at a residue corresponding to position 244 in SEQ ID NO: 1; W, F, L, Y at a residue corresponding to position 246 in SEQ ID NO: 1; F, C at a residue corresponding to position 247 in SEQ ID NO: 1; S, P at a residue corresponding to position 248 in SEQ ID NO: 1; A, V, M at a residue corresponding to position 249 in SEQ ID NO: 1; M, A, W at a residue corresponding to position 250 in SEQ ID NO: 1; A, H at a residue corresponding to position 251 in SEQ ID NO: 1; I, M, W, Q at a residue corresponding to position 252 in SEQ ID NO: 1; T, Q, N, I, H, V, M at a residue corresponding to position 253 in SEQ ID NO: 1; E, I, T, Y, H, V, L, P, A, M, F, S, G at a residue corresponding to position 254 in SEQ ID NO: 1; M, T, I, N, Y, C, ILLANV (SEQ ID NO: 35), ILLANVHY (SEQ ID NO: 36), ILLLNVHY (SEQ ID NO: 37), ILLLNV (SEQ ID NO: 38), ILLLNVHK (SEQ ID NO: 39), ILLHNVHK (SEQ ID NO: 40), ILLSNVHY (SEQ ID NO: 41) at a residue corresponding to position 255 in SEQ ID NO: 1; I, V, H, C, F, a deletion at a residue corresponding to position 256 in SEQ ID NO: 1; a deletion, L, T, N at a residue corresponding to position 257 in SEQ ID NO: 1; a deletion, S, L, P, M, H, T, A at a residue corresponding to position 258 in SEQ ID NO: 1; a deletion, C, A at a residue corresponding to position 259 in SEQ ID NO: 1; a deletion, F, L, M, V at a residue corresponding to position 260 in SEQ ID NO: 1; a deletion, Y, M, W, A, F, L, S at a residue corresponding to position 261 in SEQ ID NO: 1; a deletion, M, K, Y, R, G, C, L, V, S, N at a residue corresponding to position 262 in SEQ ID NO: 1; G, W, I, S, V at a residue corresponding to position 264 in SEQ ID NO: 1; R, C, H, K at a residue corresponding to position 265 in SEQ ID NO: 1; I, V, Q, C at a residue corresponding to position 266 in SEQ ID NO: 1; Y at a residue corresponding to position 267 in SEQ ID NO: 1; V, M, T, F, IMPR (SEQ ID NO: 42), IMNR (SEQ ID NO: 43) at a residue corresponding to position 268 in SEQ ID NO: 1; M, at a residue corresponding to position 269 in SEQ ID NO: 1; a deletion, P at a residue corresponding to position 270 in SEQ ID NO: 1; a deletion, R, Q at a residue corresponding to position 271 in SEQ ID NO: 1; W at a residue corresponding to position 272 in SEQ ID NO: 1; P, PEEMLRAIQ (SEQ ID NO: 44), PEEMLQAIQ (SEQ ID NO: 45) at a residue corresponding to position 274 in SEQ ID NO: 1; D, A, E, R, K, EEMLQAIQ (SEQ ID NO: 46), EEMLRAIQ (SEQ ID NO: 47), a deletion, KEMLQAIQ (SEQ ID NO: 48) at a residue corresponding to position 275 in SEQ ID NO: 1; a deletion at a residue corresponding to position 276 in SEQ ID NO: 1; a deletion, M, Y, L, F at a residue corresponding to position 277 in SEQ ID NO: 1; a deletion at a residue corresponding to position 278 in SEQ ID NO: 1; a deletion, Q, R, QAIQ (SEQ ID NO: 49), RAIQ (SEQ ID NO: 50) at a residue corresponding to position 279 in SEQ ID NO: 1; a deletion at a residue corresponding to position 280 in SEQ ID NO: 1; a deletion, V, M, L, I at a residue corresponding to position 281 in SEQ ID NO: 1; a deletion, Q at a residue corresponding to position 282 in SEQ ID NO: 1; P, C at a residue corresponding to position 286 in SEQ ID NO: 1; T, TVFFA (SEQ ID NO: 51), TSFFA (SEQ ID NO: 52), TFFFA (SEQ ID NO: 53), TMFFA (SEQ ID NO: 54), TVFHA (SEQ ID NO: 55) at a residue corresponding to position 287 in SEQ ID NO: 1; H, F, M, Y, R, I, V, T, C, a deletion at a residue corresponding to position 288 in SEQ ID NO: 1; a deletion, M, V, A, L, F, I at a residue corresponding to position 289 in SEQ ID NO: 1; a deletion, A, W, H, C, F, V, P, L at a residue corresponding to position 290 in SEQ ID NO: 1; a deletion, A, M, G, V at a residue corresponding to position 291 in SEQ ID NO: 1; A, T at a residue corresponding to position 294 in SEQ ID NO: 1; L, a deletion at a residue corresponding to position 295 in SEQ ID NO: 1; M, L at a residue corresponding to position 296 in SEQ ID NO: 1; M, Q, I, A, L, T at a residue corresponding to position 297 in SEQ ID NO: 1; M, A, F, R, IM at a residue corresponding to position 298 in SEQ ID NO: 1; M, F at a residue corresponding to position 299 in SEQ ID NO: 1; A at a residue corresponding to position 300 in SEQ ID NO: 1; M, R, E at a residue corresponding to position 304 in SEQ ID NO: 1; D at a residue corresponding to position 306 in SEQ ID NO: 1; L at a residue corresponding to position 309 in SEQ ID NO: 1; L, D at a residue corresponding to position 310 in SEQ ID NO: 1; L at a residue corresponding to position 311 in SEQ ID NO: 1; S, SHVRSV (SEQ ID NO: 56), SHVRTV (SEQ ID NO: 57) at a residue corresponding to position 314 in SEQ ID NO: 1; S, a deletion at a residue corresponding to position 315 in SEQ ID NO: 1; a deletion, V, L, I at a residue corresponding to position 316 in SEQ ID NO: 1; a deletion, H, N, Q, T at a residue corresponding to position 317 in SEQ ID NO: 1; a deletion, Y, M, T, S, C, Q, R, H, V, W, I, L at a residue corresponding to position 318 in SEQ ID NO: 1; a deletion, C, F, V, L, M at a residue corresponding to position 319 in SEQ ID NO: 1; F, I, V, Y, T, M, W at a residue corresponding to position 320 in SEQ ID NO: 1; C at a residue corresponding to position 321 in SEQ ID NO: 1; G at a residue corresponding to position 323 in SEQ ID NO: 1; S, M at a residue corresponding to position 324 in SEQ ID NO: 1; V, I, C at a residue corresponding to position 326 in SEQ ID NO: 1; S at a residue corresponding to position 327 in SEQ ID NO: 1; H, V, R, S, RELE (SEQ ID NO: 58) at a residue corresponding to position 328 in SEQ ID NO: 1; E, a deletion, ELE at a residue corresponding to position 329 in SEQ ID NO: 1; a deletion, L, T, I at a residue corresponding to position 330 in SEQ ID NO: 1; a deletion, M, E, C, L, I at a residue corresponding to position 331 in SEQ ID NO: 1; A, W, C, M, I at a residue corresponding to position 334 in SEQ ID NO: 1; Q, R at a residue corresponding to position 335 in SEQ ID NO: 1; K at a residue corresponding to position 336 in SEQ ID NO: 1; M at a residue corresponding to position 338 in SEQ ID NO: 1; S at a residue corresponding to position 339 in SEQ ID NO: 1; H, T at a residue corresponding to position 341 in SEQ ID NO: 1; L, F at a residue corresponding to position 342 in SEQ ID NO: 1; D, H, Y at a residue corresponding to position 348 in SEQ ID NO: 1; V, C, T, F, A, W at a residue corresponding to position 351 in SEQ ID NO: 1; E, K, D at a residue corresponding to position 352 in SEQ ID NO: 1; V, L, M at a residue corresponding to position 353 in SEQ ID NO: 1; C, F, L, Y, A, K, S, I at a residue corresponding to position 354 in SEQ ID NO: 1; E at a residue corresponding to position 355 in SEQ ID NO: 1; A at a residue corresponding to position 356 in SEQ ID NO: 1; W at a residue corresponding to position 357 in SEQ ID NO: 1; M at a residue corresponding to position 359 in SEQ ID NO: 1; S at a residue corresponding to position 360 in SEQ ID NO: 1; C, S at a residue corresponding to position 362 in SEQ ID NO: 1; A, T, G at a residue corresponding to position 363 in SEQ ID NO: 1; C, S at a residue corresponding to position 364 in SEQ ID NO: 1; L, I, M, C at a residue corresponding to position 365 in SEQ ID NO: 1; S, V, T at a residue corresponding to position 366 in SEQ ID NO: 1; A, C, S, H at a residue corresponding to position 367 in SEQ ID NO: 1; H at a residue corresponding to position 368 in SEQ ID NO: 1; H, Y, I, C, L, V, W, S, T, M at a residue corresponding to position 369 in SEQ ID NO: 1; Y, R, T, S, H, G, N, Q at a residue corresponding to position 370 in SEQ ID NO: 1; W at a residue corresponding to position 371 in SEQ ID NO: 1; K, W, Q, H, S, R, N at a residue corresponding to position 372 in SEQ ID NO: 1; S, H, P, Q, E at a residue corresponding to position 373 in SEQ ID NO: 1; D, T, A, R, S, P, W, E, Q, H at a residue corresponding to position 374 in SEQ ID NO: 1; P at a residue corresponding to position 375 in SEQ ID NO: 1; T, K, E, Q, H, D at a residue corresponding to position 376 in SEQ ID NO: 1; Y, R, N at a residue corresponding to position 377 in SEQ ID NO: 1; S, Y, P, V, A at a residue corresponding to position 378 in SEQ ID NO: 1; G at a residue corresponding to position 379 in SEQ ID NO: 1; T at a residue corresponding to position 380 in SEQ ID NO: 1; V, S, I at a residue corresponding to position 381 in SEQ ID NO: 1; Q, H, T, Y, W, P at a residue corresponding to position 383 in SEQ ID NO: 1; I, P, A, M at a residue corresponding to position 384 in SEQ ID NO: 1; S, A at a residue corresponding to position 386 in SEQ ID NO: 1; G, S, H at a residue corresponding to position 387 in SEQ ID NO: 1; T, M, V, L at a residue corresponding to position 388 in SEQ ID NO: 1; R, K, E at a residue corresponding to position 389 in SEQ ID NO: 1; C, L, A at a residue corresponding to position 390 in SEQ ID NO: 1; K at a residue corresponding to position 391 in SEQ ID NO: 1; D at a residue corresponding to position 394 in SEQ ID NO: 1; D, N at a residue corresponding to position 398 in SEQ ID NO: 1; V, L, I at a residue corresponding to position 402 in SEQ ID NO: 1; L, V at a residue corresponding to position 406 in SEQ ID NO: 1; D at a residue corresponding to position 407 in SEQ ID NO: 1; N, Q, NTENAPR (SEQ ID NO: 59), NTENAPD (SEQ ID NO: 60) at a residue corresponding to position 410 in SEQ ID NO: 1; T, TENAPD (SEQ ID NO: 61), TQNAPR (SEQ ID NO: 62), a deletion, TENAPR (SEQ ID NO: 63), TQNAPN (SEQ ID NO: 64) at a residue corresponding to position 411 in SEQ ID NO: 1; a deletion, Q at a residue corresponding to position 412 in SEQ ID NO: 1; a deletion, N, NNPN (SEQ ID NO: 65) at a residue corresponding to position 413 in SEQ ID NO: 1; a deletion, A, S, N at a residue corresponding to position 414 in SEQ ID NO: 1; a deletion, P at a residue corresponding to position 415 in SEQ ID NO: 1; a deletion, D, R, N, E at a residue corresponding to position 416 in SEQ ID NO: 1; P at a residue corresponding to position 418 in SEQ ID NO: 1; G at a residue corresponding to position 419 in SEQ ID NO: 1; E at a residue corresponding to position 420 in SEQ ID NO: 1; M, V at a residue corresponding to position 424 in SEQ ID NO: 1; V, L at a residue corresponding to position 425 in SEQ ID NO: 1; M, L at a residue corresponding to position 426 in SEQ ID NO: 1; T, N, S at a residue corresponding to position 430 in SEQ ID NO: 1; I, L at a residue corresponding to position 431 in SEQ ID NO: 1; T, F at a residue corresponding to position 432 in SEQ ID NO: 1; S, K at a residue corresponding to position 433 in SEQ ID NO: 1; W at a residue corresponding to position 436 in SEQ ID NO: 1; T at a residue corresponding to position 442 in SEQ ID NO: 1; R, K, RASFA (SEQ ID NO: 66) at a residue corresponding to position 443 in SEQ ID NO: 1; a deletion at a residue corresponding to position 444 in SEQ ID NO: 1; a deletion, M, T, S, A at a residue corresponding to position 445 in SEQ ID NO: 1; a deletion at a residue corresponding to position 446 in SEQ ID NO: 1; a deletion, A at a residue corresponding to position 447 in SEQ ID NO: 1; S, G at a residue corresponding to position 455 in SEQ ID NO: 1; G, E, D at a residue corresponding to position 459 in SEQ ID NO: 1; D, G at a residue corresponding to position 461 in SEQ ID NO: 1; R, G at a residue corresponding to position 462 in SEQ ID NO: 1; L, Y at a residue corresponding to position 464 in SEQ ID NO: 1; H at a residue corresponding to position 465 in SEQ ID NO: 1; M at a residue corresponding to position 469 in SEQ ID NO: 1; C, G at a residue corresponding to position 470 in SEQ ID NO: 1; Y, K, M, W, I, R at a residue corresponding to position 471 in SEQ ID NO: 1; I, F, C, Y, L, M at a residue corresponding to position 472 in SEQ ID NO: 1; E, EDGH (SEQ ID NO: 67), EDGY (SEQ ID NO: 68) at a residue corresponding to position 474 in SEQ ID NO: 1; D, a deletion at a residue corresponding to position 475 in SEQ ID NO: 1; a deletion at a residue corresponding to position 476 in SEQ ID NO: 1; a deletion, H, F, N, E, Y at a residue corresponding to position 477 in SEQ ID NO: 1; W, I, M at a residue corresponding to position 478 in SEQ ID NO: 1; T, H, Y, F, W at a residue corresponding to position 479 in SEQ ID NO: 1; V, L at a residue corresponding to position 480 in SEQ ID NO: 1; C, I, T at a residue corresponding to position 481 in SEQ ID NO: 1; L at a residue corresponding to position 484 in SEQ ID NO: 1; D at a residue corresponding to position 486 in SEQ ID NO: 1; I at a residue corresponding to position 489 in SEQ ID NO: 1; R, C, V at a residue corresponding to position 490 in SEQ ID NO: 1; G, R, N at a residue corresponding to position 491 in SEQ ID NO: 1; F, H, Y, L at a residue corresponding to position 493 in SEQ ID NO: 1; N at a residue corresponding to position 494 in SEQ ID NO: 1; F, S, Y at a residue corresponding to position 496 in SEQ ID NO: 1; L, V, I at a residue corresponding to position 503 in SEQ ID NO: 1; R, RHPD (SEQ ID NO: 69) at a residue corresponding to position 506 in SEQ ID NO: 1; a deletion at a residue corresponding to position 507 in SEQ ID NO: 1; a deletion, P at a residue corresponding to position 508 in SEQ ID NO: 1; a deletion, D at a residue corresponding to position 509 in SEQ ID NO: 1; A, V, C at a residue corresponding to position 513 in SEQ ID NO: 1; V at a residue corresponding to position 518 in SEQ ID NO: 1; P at a residue corresponding to position 519 in SEQ ID NO: 1; E, P at a residue corresponding to position 521 in SEQ ID NO: 1; Q, A, R, M, Y, D at a residue corresponding to position 523 in SEQ ID NO: 1; S, A at a residue corresponding to position 524 in SEQ ID NO: 1; S, T, D, G, E at a residue corresponding to position 525 in SEQ ID NO: 1; P, T, V at a residue corresponding to position 527 in SEQ ID NO: 1; A, V at a residue corresponding to position 528 in SEQ ID NO: 1; V, A at a residue corresponding to position 529 in SEQ ID NO: 1; L, V at a residue corresponding to position 539 in SEQ ID NO: 1; T at a residue corresponding to position 540 in SEQ ID NO: 1; E, D at a residue corresponding to position 542 in SEQ ID NO: 1; A, K at a residue corresponding to position 546 in SEQ ID NO: 1; K, R at a residue corresponding to position 551 in SEQ ID NO: 1; S at a residue corresponding to position 553 in SEQ ID NO: 1; S, P, H at a residue corresponding to position 554 in SEQ ID NO: 1; F at a residue corresponding to position 555 in SEQ ID NO: 1; K at a residue corresponding to position 557 in SEQ ID NO: 1; P at a residue corresponding to position 559 in SEQ ID NO: 1; G at a residue corresponding to position 560 in SEQ ID NO: 1; V, a deletion at a residue corresponding to position 562 in SEQ ID NO: 1; F at a residue corresponding to position 564 in SEQ ID NO: 1; V, F, FV at a residue corresponding to position 565 in SEQ ID NO: 1; E, VE at a residue corresponding to position 566 in SEQ ID NO: 1; Q, E at a residue corresponding to position 583 in SEQ ID NO: 1; R, E at a residue corresponding to position 5 in SEQ ID NO: 1; L at a residue corresponding to position 6 in SEQ ID NO: 1; K, T at a residue corresponding to position 7 in SEQ ID NO: 1; P at a residue corresponding to position 8 in SEQ ID NO: 1; K, D, P, N, R at a residue corresponding to position 10 in SEQ ID NO: 1; A, I, V, M at a residue corresponding to position 12 in SEQ ID NO: 1; C at a residue corresponding to position 13 in SEQ ID NO: 1; P, A, Q at a residue corresponding to position 14 in SEQ ID NO: 1; E, L, S, R at a residue corresponding to position 15 in SEQ ID NO: 1; C, L, I at a residue corresponding to position 16 in SEQ ID NO: 1; D, R at a residue corresponding to position 19 in SEQ ID NO: 1; H, K, R, D at a residue corresponding to position 20 in SEQ ID NO: 1; D, E at a residue corresponding to position 22 in SEQ ID NO: 1; N at a residue corresponding to position 25 in SEQ ID NO: 1; M at a residue corresponding to position 28 in SEQ ID NO: 1; C at a residue corresponding to position 37 in SEQ ID NO: 1; R at a residue corresponding to position 39 in SEQ ID NO: 1; T at a residue corresponding to position 40 in SEQ ID NO: 1; M, L, V, I at a residue corresponding to position 41 in SEQ ID NO: 1; S at a residue corresponding to position 43 in SEQ ID NO: 1; M at a residue corresponding to position 44 in SEQ ID NO: 1; I, V at a residue corresponding to position 45 in SEQ ID NO: 1; P at a residue corresponding to position 48 in SEQ ID NO: 1; I, F, R, V, L at a residue corresponding to position 50 in SEQ ID NO: 1; R at a residue corresponding to position 51 in SEQ ID NO: 1; L at a residue corresponding to position 53 in SEQ ID NO: 1; F at a residue corresponding to position 57 in SEQ ID NO: 1; V, L, M, F at a residue corresponding to position 58 in SEQ ID NO: 1; V at a residue corresponding to position 59 in SEQ ID NO: 1; P, E at a residue corresponding to position 61 in SEQ ID NO: 1; D at a residue corresponding to position 62 in SEQ ID NO: 1; D at a residue corresponding to position 63 in SEQ ID NO: 1; G at a residue corresponding to position 66 in SEQ ID NO: 1; K at a residue corresponding to position 67 in SEQ ID NO: 1; F, Y, L at a residue corresponding to position 69 in SEQ ID NO: 1; F, W at a residue corresponding to position 71 in SEQ ID NO: 1; A, R at a residue corresponding to position 72 in SEQ ID NO: 1; F at a residue corresponding to position 74 in SEQ ID NO: 1; D at a residue corresponding to position 75 in SEQ ID NO: 1; T, D, A, R at a residue corresponding to position 76 in SEQ ID NO: 1; R at a residue corresponding to position 77 in SEQ ID NO: 1; A at a residue corresponding to position 78 in SEQ ID NO: 1; K at a residue corresponding to position 80 in SEQ ID NO: 1; C at a residue corresponding to position 81 in SEQ ID NO: 1; S, H, Q, N at a residue corresponding to position 83 in SEQ ID NO: 1; Y, H at a residue corresponding to position 84 in SEQ ID NO: 1; F at a residue corresponding to position 85 in SEQ ID NO: 1; A, Q at a residue corresponding to position 86 in SEQ ID NO: 1; R at a residue corresponding to position 87 in SEQ ID NO: 1; L at a residue corresponding to position 88 in SEQ ID NO: 1; K at a residue corresponding to position 92 in SEQ ID NO: 1; G at a residue corresponding to position 93 in SEQ ID NO: 1; R at a residue corresponding to position 94 in SEQ ID NO: 1; R at a residue corresponding to position 95 in SEQ ID NO: 1; A at a residue corresponding to position 97 in SEQ ID NO: 1; M at a residue corresponding to position 98 in SEQ ID NO: 1; L at a residue corresponding to position 99 in SEQ ID NO: 1; C at a residue corresponding to position 100 in SEQ ID NO: 1; P at a residue corresponding to position 101 in SEQ ID NO: 1; R at a residue corresponding to position 103 in SEQ ID NO: 1; L, W, A at a residue corresponding to position 104 in SEQ ID NO: 1; W, F at a residue corresponding to position 106 in SEQ ID NO: 1; V, P, I, L, W, F at a residue corresponding to position 107 in SEQ ID NO: 1; V at a residue corresponding to position 108 in SEQ ID NO: 1; V at a residue corresponding to position 109 in SEQ ID NO: 1; M, F, Y, A, D at a residue corresponding to position 110 in SEQ ID NO: 1; T, C, V at a residue corresponding to position 113 in SEQ ID NO: 1; M, W, L, G, A at a residue corresponding to position 114 in SEQ ID NO: 1; A at a residue corresponding to position 115 in SEQ ID NO: 1; I at a residue corresponding to position 116 in SEQ ID NO: 1; V, T, I at a residue corresponding to position 119 in SEQ ID NO: 1; M, F, V, A, L, W, Y at a residue corresponding to position 120 in SEQ ID NO: 1; T at a residue corresponding to position 121 in SEQ ID NO: 1; A at a residue corresponding to position 122 in SEQ ID NO: 1; M, V, I at a residue corresponding to position 123 in SEQ ID NO: 1; Y, S, N at a residue corresponding to position 124 in SEQ ID NO: 1; P, T, W at a residue corresponding to position 125 in SEQ ID NO: 1; M, N, R, A at a residue corresponding to position 126 in SEQ ID NO: 1; F, W, L at a residue corresponding to position 127 in SEQ ID NO: 1; R at a residue corresponding to position 128 in SEQ ID NO: 1; M at a residue corresponding to position 130 in SEQ ID NO: 1; E at a residue corresponding to position 131 in SEQ ID NO: 1; E, Q, K at a residue corresponding to position 136 in SEQ ID NO: 1; M at a residue corresponding to position 137 in SEQ ID NO: 1; M, C, I at a residue corresponding to position 140 in SEQ ID NO: 1; M at a residue corresponding to position 141 in SEQ ID NO: 1; N, Q at a residue corresponding to position 142 in SEQ ID NO: 1; G, R, D at a residue corresponding to position 145 in SEQ ID NO: 1; S at a residue corresponding to position 147 in SEQ ID NO: 1; L, V, A at a residue corresponding to position 148 in SEQ ID NO: 1; V, L at a residue corresponding to position 149 in SEQ ID NO: 1; V at a residue corresponding to position 150 in SEQ ID NO: 1; C at a residue corresponding to position 151 in SEQ ID NO: 1; L at a residue corresponding to position 157 in SEQ ID NO: 1; R, E at a residue corresponding to position 159 in SEQ ID NO: 1; I at a residue corresponding to position 161 in SEQ ID NO: 1; V at a residue corresponding to position 163 in SEQ ID NO: 1; A at a residue corresponding to position 164 in SEQ ID NO: 1; R at a residue corresponding to position 165 in SEQ ID NO: 1; R at a residue corresponding to position 167 in SEQ ID NO: 1; H at a residue corresponding to position 168 in SEQ ID NO: 1; I at a residue corresponding to position 169 in SEQ ID NO: 1; I, V, L at a residue corresponding to position 170 in SEQ ID NO: 1; V at a residue corresponding to position 173 in SEQ ID NO: 1; R at a residue corresponding to position 175 in SEQ ID NO: 1; D at a residue corresponding to position 176 in SEQ ID NO: 1; E at a residue corresponding to position 177 in SEQ ID NO: 1; E at a residue corresponding to position 181 in SEQ ID NO: 1; I at a residue corresponding to position 183 in SEQ ID NO: 1; S at a residue corresponding to position 185 in SEQ ID NO: 1; Y at a residue corresponding to position 186 in SEQ ID NO: 1; D at a residue corresponding to position 187 in SEQ ID NO: 1; V at a residue corresponding to position 189 in SEQ ID NO: 1; V at a residue corresponding to position 190 in SEQ ID NO: 1; A, E at a residue corresponding to position 191 in SEQ ID NO: 1; T at a residue corresponding to position 192 in SEQ ID NO: 1; A at a residue corresponding to position 193 in SEQ ID NO: 1; S at a residue corresponding to position 194 in SEQ ID NO: 1; S at a residue corresponding to position 195 in SEQ ID NO: 1; P at a residue corresponding to position 196 in SEQ ID NO: 1; A, P, L at a residue corresponding to position 201 in SEQ ID NO: 1; T at a residue corresponding to position 205 in SEQ ID NO: 1; D at a residue corresponding to position 208 in SEQ ID NO: 1; I, T, P, V at a residue corresponding to position 209 in SEQ ID NO: 1; L, T, C, M, V, A at a residue corresponding to position 211 in SEQ ID NO: 1; L, M at a residue corresponding to position 212 in SEQ ID NO: 1; I, C, V, Q, M at a residue corresponding to position 213 in SEQ ID NO: 1; Y at a residue corresponding to position 214 in SEQ ID NO: 1; S at a residue corresponding to position 215 in SEQ ID NO: 1; G at a residue corresponding to position 216 in SEQ ID NO: 1; T at a residue corresponding to position 218 in SEQ ID NO: 1; T at a residue corresponding to position 219 in SEQ ID NO: 1; G at a residue corresponding to position 220 in SEQ ID NO: 1; T, M, L, R, K, H at a residue corresponding to position 221 in SEQ ID NO: 1; S at a residue corresponding to position 222 in SEQ ID NO: 1; A at a residue corresponding to position 224 in SEQ ID NO: 1; A at a residue corresponding to position 225 in SEQ ID NO: 1; M, I, V at a residue corresponding to position 226 in SEQ ID NO: 1; L, D, H, I at a residue corresponding to position 227 in SEQ ID NO: 1; S at a residue corresponding to position 228 in SEQ ID NO: 1; H, K at a residue corresponding to position 230 in SEQ ID NO: 1; N at a residue corresponding to position 231 in SEQ ID NO: 1; L, I, V at a residue corresponding to position 232 in SEQ ID NO: 1; L, V, M, W, A, I at a residue corresponding to position 233 in SEQ ID NO: 1; S, W at a residue corresponding to position 234 in SEQ ID NO: 1; Q, S at a residue corresponding to position 235 in SEQ ID NO: 1; A, C, I, M, V at a residue corresponding to position 236 in SEQ ID NO: 1; A, M, Q, R, E at a residue corresponding to position 237 in SEQ ID NO: 1; M, S, W, N, H at a residue corresponding to position 238 in SEQ ID NO: 1; F, M, V, C, L at a residue corresponding to position 239 in SEQ ID NO: 1; A, R at a residue corresponding to position 240 in SEQ ID NO: 1; A, H, E, Q at a residue corresponding to position 241 in SEQ ID NO: 1; V, C, T, W at a residue corresponding to position 242 in SEQ ID NO: 1; I, F, M, W, V at a residue corresponding to position 243 in SEQ ID NO: 1; E, P, Q, H, G at a residue corresponding to position 244 in SEQ ID NO: 1; M, L, H, W, Y at a residue corresponding to position 245 in SEQ ID NO: 1; I, T, M at a residue corresponding to position 246 in SEQ ID NO: 1; R, P, E at a residue corresponding to position 247 in SEQ ID NO: 1; E at a residue corresponding to position 251 in SEQ ID NO: 1; H, V at a residue corresponding to position 252 in SEQ ID NO: 1; Y, M, F, L, V at a residue corresponding to position 253 in SEQ ID NO: 1; I, F, V, M at a residue corresponding to position 254 in SEQ ID NO: 1; A, T, N, L, I, G, H, C, V, S at a residue corresponding to position 255 in SEQ ID NO: 1; F, V, I, C, Y at a residue corresponding to position 256 in SEQ ID NO: 1; M at a residue corresponding to position 257 in SEQ ID NO: 1; L, F at a residue corresponding to position 259 in SEQ ID NO: 1; H, S, Y, F at a residue corresponding to position 260 in SEQ ID NO: 1; C, T, A, I, M at a residue corresponding to position 262 in SEQ ID NO: 1; A, Y, H at a residue corresponding to position 263 in SEQ ID NO: 1; A at a residue corresponding to position 264 in SEQ ID NO: 1; M, L, W, F, C at a residue corresponding to position 265 in SEQ ID NO: 1; N, T, M, V at a residue corresponding to position 266 in SEQ ID NO: 1; I, G, M, W, F, L, V, C, A at a residue corresponding to position 267 in SEQ ID NO: 1; M, A, C, G, V at a residue corresponding to position 268 in SEQ ID NO: 1; L, M, I, V at a residue corresponding to position 269 in SEQ ID NO: 1; M, H, Y, T at a residue corresponding to position 270 in SEQ ID NO: 1; A, P, C, T, L at a residue corresponding to position 271 in SEQ ID NO: 1; C, V, P, A, T at a residue corresponding to position 272 in SEQ ID NO: 1; M, V, F, I at a residue corresponding to position 273 in SEQ ID NO: 1; Y, R, V, M, C, H, W at a residue corresponding to position 274 in SEQ ID NO: 1; G, C, S, H at a residue corresponding to position 275 in SEQ ID NO: 1; A, C at a residue corresponding to position 277 in SEQ ID NO: 1; C, T, A, H at a residue corresponding to position 278 in SEQ ID NO: 1; V, M, I at a residue corresponding to position 279 in SEQ ID NO: 1; F, L, V, M at a residue corresponding to position 281 in SEQ ID NO: 1; P, M, E, A, H at a residue corresponding to position 282 in SEQ ID NO: 1; E, D, P, R at a residue corresponding to position 283 in SEQ ID NO: 1; F, R, W at a residue corresponding to position 284 in SEQ ID NO: 1; R, F, W, D at a residue corresponding to position 285 in SEQ ID NO: 1; D, F at a residue corresponding to position 286 in SEQ ID NO: 1; P, A, V at a residue corresponding to position 287 in SEQ ID NO: 1; A at a residue corresponding to position 290 in SEQ ID NO: 1; M, V, L, P, I at a residue corresponding to position 291 in SEQ ID NO: 1; H at a residue corresponding to position 292 in SEQ ID NO: 1; I, L, A at a residue corresponding to position 294 in SEQ ID NO: 1; W, M, V, I at a residue corresponding to position 295 in SEQ ID NO: 1; D, Q at a residue corresponding to position 296 in SEQ ID NO: 1; M, V, L at a residue corresponding to position 298 in SEQ ID NO: 1; A at a residue corresponding to position 299 in SEQ ID NO: 1; Q at a residue corresponding to position 300 in SEQ ID NO: 1; Q at a residue corresponding to position 302 in SEQ ID NO: 1; V, I, A, P, C at a residue corresponding to position 303 in SEQ ID NO: 1; S at a residue corresponding to position 304 in SEQ ID NO: 1; W, Y, V, I, F, H, M at a residue corresponding to position 305 in SEQ ID NO: 1; V, Y, I, M, L, F at a residue corresponding to position 306 in SEQ ID NO: 1; P, W, Y, H, M at a residue corresponding to position 307 in SEQ ID NO: 1; T, A, S, L, G, V, M at a residue corresponding to position 308 in SEQ ID NO: 1; A, R, P, T at a residue corresponding to position 311 in SEQ ID NO: 1; I, M, V, F at a residue corresponding to position 312 in SEQ ID NO: 1; L, F, W at a residue corresponding to position 313 in SEQ ID NO: 1; Q, E, H at a residue corresponding to position 314 in SEQ ID NO: 1; R, A, K, L at a residue corresponding to position 315 in SEQ ID NO: 1; W, M, I, F at a residue corresponding to position 316 in SEQ ID NO: 1; H, D at a residue corresponding to position 318 in SEQ ID NO: 1; R, Q at a residue corresponding to position 321 in SEQ ID NO: 1; Q, K, A, H at a residue corresponding to position 322 in SEQ ID NO: 1; L, F at a residue corresponding to position 330 in SEQ ID NO: 1; G at a residue corresponding to position 331 in SEQ ID NO: 1; S at a residue corresponding to position 333 in SEQ ID NO: 1; I, M, V at a residue corresponding to position 334 in SEQ ID NO: 1; R, K at a residue corresponding to position 335 in SEQ ID NO: 1; T, I, M, Y, V, F, W at a residue corresponding to position 336 in SEQ ID NO: 1; L, M, F, A, V at a residue corresponding to position 337 in SEQ ID NO: 1; W, L, M, V at a residue corresponding to position 338 in SEQ ID NO: 1; F, T, S, C at a residue corresponding to position 339 in SEQ ID NO: 1; G at a residue corresponding to position 340 in SEQ ID NO: 1; A, S at a residue corresponding to position 341 in SEQ ID NO: 1; S at a residue corresponding to position 342 in SEQ ID NO: 1; P, A at a residue corresponding to position 343 in SEQ ID NO: 1; M, I, V, C at a residue corresponding to position 344 in SEQ ID NO: 1; S at a residue corresponding to position 345 in SEQ ID NO: 1; V, A, P, E at a residue corresponding to position 346 in SEQ ID NO: 1; E at a residue corresponding to position 347 in SEQ ID NO: 1; T, I, L at a residue corresponding to position 348 in SEQ ID NO: 1; M, F, L, A, I, Y at a residue corresponding to position 349 in SEQ ID NO: 1; F, R, Q, W at a residue corresponding to position 351 in SEQ ID NO: 1; I, M, Y at a residue corresponding to position 352 in SEQ ID NO: 1; R at a residue corresponding to position 353 in SEQ ID NO: 1; K at a residue corresponding to position 354 in SEQ ID NO: 1; A, R at a residue corresponding to position 355 in SEQ ID NO: 1; W, G, V, L, P, I, F, A at a residue corresponding to position 357 in SEQ ID NO: 1; Q at a residue corresponding to position 358 in SEQ ID NO: 1; H, Q at a residue corresponding to position 359 in SEQ ID NO: 1; F at a residue corresponding to position 361 in SEQ ID NO: 1; H, L, C at a residue corresponding to position 362 in SEQ ID NO: 1; D, V, I, G at a residue corresponding to position 363 in SEQ ID NO: 1; M, V, I at a residue corresponding to position 365 in SEQ ID NO: 1; I, V, L, F at a residue corresponding to position 366 in SEQ ID NO: 1; T, C, M, I, V, L at a residue corresponding to position 367 in SEQ ID NO: 1; N, Q, E, T at a residue corresponding to position 368 in SEQ ID NO: 1; G, A at a residue corresponding to position 369 in SEQ ID NO: 1; Y, F at a residue corresponding to position 370 in SEQ ID NO: 1; L, Q at a residue corresponding to position 372 in SEQ ID NO: 1; S at a residue corresponding to position 373 in SEQ ID NO: 1; A, C at a residue corresponding to position 375 in SEQ ID NO: 1; T, S, C, G at a residue corresponding to position 376 in SEQ ID NO: 1; G, M at a residue corresponding to position 377 in SEQ ID NO: 1; C, T, V at a residue corresponding to position 378 in SEQ ID NO: 1; C, V, I, L, A at a residue corresponding to position 379 in SEQ ID NO: 1; F, C, H, S, M, T, V at a residue corresponding to position 380 in SEQ ID NO: 1; N, F, L, C, V at a residue corresponding to position 381 in SEQ ID NO: 1; L, N, C, T, P at a residue corresponding to position 382 in SEQ ID NO: 1; D, N, R, P, Y at a residue corresponding to position 383 in SEQ ID NO: 1; P, N, D, W at a residue corresponding to position 384 in SEQ ID NO: 1; G, D at a residue corresponding to position 385 in SEQ ID NO: 1; D, V, L, T at a residue corresponding to position 386 in SEQ ID NO: 1; D, R, N, H, E at a residue corresponding to position 387 in SEQ ID NO: 1; K, H at a residue corresponding to position 388 in SEQ ID NO: 1; A, P, L at a residue corresponding to position 389 in SEQ ID NO: 1; S, T at a residue corresponding to position 391 in SEQ ID NO: 1; V, C at a residue corresponding to position 392 in SEQ ID NO: 1; R, K, I, Q, L at a residue corresponding to position 394 in SEQ ID NO: 1; A at a residue corresponding to position 395 in SEQ ID NO: 1; F, I, M, V at a residue corresponding to position 396 in SEQ ID NO: 1; D, H, N at a residue corresponding to position 398 in SEQ ID NO: 1; M, V, T, C, I at a residue corresponding to position 399 in SEQ ID NO: 1; D, Q at a residue corresponding to position 400 in SEQ ID NO: 1; M, I, C, L, A at a residue corresponding to position 401 in SEQ ID NO: 1; R at a residue corresponding to position 402 in SEQ ID NO: 1; V at a residue corresponding to position 404 in SEQ ID NO: 1; D at a residue corresponding to position 405 in SEQ ID NO: 1; E at a residue corresponding to position 406 in SEQ ID NO: 1; P at a residue corresponding to position 407 in SEQ ID NO: 1; Q, E, G at a residue corresponding to position 409 in SEQ ID NO: 1; D, S, V, G, N at a residue corresponding to position 411 in SEQ ID NO: 1; I, V at a residue corresponding to position 413 in SEQ ID NO: 1; W, L, V, C, Q at a residue corresponding to position 414 in SEQ ID NO: 1; I at a residue corresponding to position 415 in SEQ ID NO: 1; R at a residue corresponding to position 416 in SEQ ID NO: 1; S at a residue corresponding to position 417 in SEQ ID NO: 1; D at a residue corresponding to position 418 in SEQ ID NO: 1; H, S, Q, M at a residue corresponding to position 419 in SEQ ID NO: 1; V at a residue corresponding to position 420 in SEQ ID NO: 1; T, F at a residue corresponding to position 421 in SEQ ID NO: 1; H, L at a residue corresponding to position 425 in SEQ ID NO: 1; A at a residue corresponding to position 430 in SEQ ID NO: 1; K at a residue corresponding to position 432 in SEQ ID NO: 1; R, D at a residue corresponding to position 433 in SEQ ID NO: 1; T, V at a residue corresponding to position 434 in SEQ ID NO: 1; I, M, L at a residue corresponding to position 435 in SEQ ID NO: 1; F, M, Y at a residue corresponding to position 441 in SEQ ID NO: 1; K, H, Y, R, C at a residue corresponding to position 442 in SEQ ID NO: 1; S at a residue corresponding to position 443 in SEQ ID NO: 1; M, I, L, V at a residue corresponding to position 446 in SEQ ID NO: 1; A, G at a residue corresponding to position 447 in SEQ ID NO: 1; I at a residue corresponding to position 449 in SEQ ID NO: 1; D, M at a residue corresponding to position 450 in SEQ ID NO: 1; E at a residue corresponding to position 451 in SEQ ID NO: 1; P at a residue corresponding to position 452 in SEQ ID NO: 1; G at a residue corresponding to position 453 in SEQ ID NO: 1; E, G, R at a residue corresponding to position 455 in SEQ ID NO: 1; D, E, Q, N at a residue corresponding to position 456 in SEQ ID NO: 1; N, F, Y, W at a residue corresponding to position 458 in SEQ ID NO: 1; Y at a residue corresponding to position 459 in SEQ ID NO: 1; Y, F, I, R, W at a residue corresponding to position 460 in SEQ ID NO: 1; I, L, Y at a residue corresponding to position 461 in SEQ ID NO: 1; I, T, V at a residue corresponding to position 462 in SEQ ID NO: 1; D at a residue corresponding to position 463 in SEQ ID NO: 1; D at a residue corresponding to position 464 in SEQ ID NO: 1; L at a residue corresponding to position 465 in SEQ ID NO: 1; D, K, A at a residue corresponding to position 466 in SEQ ID NO: 1; K at a residue corresponding to position 467 in SEQ ID NO: 1; M at a residue corresponding to position 468 in SEQ ID NO: 1; L at a residue corresponding to position 469 in SEQ ID NO: 1; V at a residue corresponding to position 470 in SEQ ID NO: 1; T, V at a residue corresponding to position 471 in SEQ ID NO: 1; S, N at a residue corresponding to position 473 in SEQ ID NO: 1; E at a residue corresponding to position 474 in SEQ ID NO: 1; N, Y, L, F, V, I at a residue corresponding to position 476 in SEQ ID NO: 1; T, L at a residue corresponding to position 478 in SEQ ID NO: 1; P, V at a residue corresponding to position 479 in SEQ ID NO: 1; L, I, S, P, A, V at a residue corresponding to position 480 in SEQ ID NO: 1; P, E at a residue corresponding to position 481 in SEQ ID NO: 1; E at a residue corresponding to position 483 in SEQ ID NO: 1; A, L, E, R at a residue corresponding to position 485 in SEQ ID NO: 1; C at a residue corresponding to position 490 in SEQ ID NO: 1; P at a residue corresponding to position 492 in SEQ ID NO: 1; F at a residue corresponding to position 493 in SEQ ID NO: 1; H at a residue corresponding to position 496 in SEQ ID NO: 1; A at a residue corresponding to position 497 in SEQ ID NO: 1; C at a residue corresponding to position 498 in SEQ ID NO: 1; P at a residue corresponding to position 502 in SEQ ID NO: 1; H at a residue corresponding to position 504 in SEQ ID NO: 1; Y, V, W at a residue corresponding to position 507 in SEQ ID NO: 1; V at a residue corresponding to position 508 in SEQ ID NO: 1; A at a residue corresponding to position 510 in SEQ ID NO: 1; V, L at a residue corresponding to position 511 in SEQ ID NO: 1; I at a residue corresponding to position 512 in SEQ ID NO: 1; V at a residue corresponding to position 513 in SEQ ID NO: 1; N at a residue corresponding to position 514 in SEQ ID NO: 1; D, S at a residue corresponding to position 515 in SEQ ID NO: 1; A at a residue corresponding to position 516 in SEQ ID NO: 1; I at a residue corresponding to position 518 in SEQ ID NO: 1; T at a residue corresponding to position 519 in SEQ ID NO: 1; P, W at a residue corresponding to position 521 in SEQ ID NO: 1; L, M at a residue corresponding to position 526 in SEQ ID NO: 1; L at a residue corresponding to position 527 in SEQ ID NO: 1; R, H at a residue corresponding to position 532 in SEQ ID NO: 1; L at a residue corresponding to position 535 in SEQ ID NO: 1; Q at a residue corresponding to position 540 in SEQ ID NO: 1; Y, W at a residue corresponding to position 542 in SEQ ID NO: 1; N at a residue corresponding to position 546 in SEQ ID NO: 1; Q at a residue corresponding to position 552 in SEQ ID NO: 1; E at a residue corresponding to position 554 in SEQ ID NO: 1; F at a residue corresponding to position 558 in SEQ ID NO: 1; D at a residue corresponding to position 560 in SEQ ID NO: 1; D at a residue corresponding to position 561 in SEQ ID NO: 1; F, L, W at a residue corresponding to position 563 in SEQ ID NO: 1; D, K, S, L, T, R at a residue corresponding to position 564 in SEQ ID NO: 1; M, I, V, P, T at a residue corresponding to position 565 in SEQ ID NO: 1; A, E, D, P at a residue corresponding to position 566 in SEQ ID NO: 1; K, Q, T, N, S, A at a residue corresponding to position 567 in SEQ ID NO: 1; G, N, D at a residue corresponding to position 568 in SEQ ID NO: 1; M, K at a residue corresponding to position 569 in SEQ ID NO: 1; V, L at a residue corresponding to position 570 in SEQ ID NO: 1; R, P at a residue corresponding to position 572 in SEQ ID NO: 1; T, S at a residue corresponding to position 573 in SEQ ID NO: 1; M, L at a residue corresponding to position 574 in SEQ ID NO: 1; A, Y, K, T at a residue corresponding to position 575 in SEQ ID NO: 1; Y, A, L at a residue corresponding to position 577 in SEQ ID NO: 1; Q, A at a residue corresponding to position 578 in SEQ ID NO: 1; D at a residue corresponding to position 583 in SEQ ID NO: 1; N at a residue corresponding to position 584 in SEQ ID NO: 1; H, L at a residue corresponding to position 585 in SEQ ID NO: 1; A at a residue corresponding to position 587 in SEQ ID NO: 1; Q at a residue corresponding to position 588 in SEQ ID NO: 1; Q, E at a residue corresponding to position 591 in SEQ ID NO: 1; A at a residue corresponding to position 593 in SEQ ID NO: 1; E at a residue corresponding to position 595 in SEQ ID NO: 1; K at a residue corresponding to position 596 in SEQ ID NO: 1; D at a residue corresponding to position 598 in SEQ ID NO: 1; S at a residue corresponding to position 599 in SEQ ID NO: 1; L at a residue corresponding to position 601 in SEQ ID NO: 1; or any combination thereof. It is understood that when listed herein, alterations listed for a certain position can be in the alternative or in the conjunctive depending on the context. For example, the V, F, FV alterations at a residue corresponding to position 565 in SEQ ID NO: 1, indicates that alterations at a residue corresponding to position 565 in SEQ ID NO: 1 can comprise V or F or FV, wherein FV is F and V.

In some embodiments, provided herein is microbial organism (e.g., host microbial organism) that has a recombinant nucleic acid encoding an engineered ACL an engineered ACL provided herein has an amino acid sequence that is a variant of SEQ ID NO: 1 that includes one or more alterations as described in TABLE 8 and/or one or more or a combination of alterations described in TABLE 9, wherein the portion, other than the one or more alterations described in TABLE 8 or the one or more or combination of alterations described in TABLE 9, of the engineered ACL has at least 65%, 70%, 75%, 80%, 85%, 90%, 95%, 98% or 99% sequence identity, or is identical, to an amino acid sequence referenced as SEQ ID NO: 1. Accordingly, in some embodiments, provided herein is microbial organism (e.g., host microbial organism) that has a recombinant nucleic acid encoding an engineered ACL that has an amino acid sequence that includes one or more alterations as described in TABLE 8 and/or one or more or a combination of alterations described in TABLE 9 and the portion, other than the alteration described in TABLE 8 or the one or more or combination of alterations described in TABLE 9, of the engineered ACL has at least 65% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and/or one or more or a combination of alterations described in TABLE 9 and the portion, other than the alteration described in TABLE 8 or the one or more or combination of alterations described in TABLE 9, of the engineered ACL has at least 70% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and/or one or more or a combination of alterations described in TABLE 9 and the portion, other than the alteration described in TABLE 8 or the one or more or combination of alterations described in TABLE 9, of the engineered ACL has at least 75% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and/or one or more or a combination of alterations described in TABLE 9 and the portion, other than the alteration described in TABLE 8 or the one or more or combination of alterations described in TABLE 9, of the engineered ACL has at least 80% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and/or one or more or a combination of alterations described in TABLE 9 and the portion, other than the alteration described in TABLE 8 or the one or more or combination of alterations described in TABLE 9, of the engineered ACL has at least 85% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and/or one or more or a combination of alterations described in TABLE 9 and the portion, other than the alteration described in TABLE 8 or the one or more or combination of alterations described in TABLE 9, of the engineered ACL has at least 90% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and/or one or more or a combination of alterations described in TABLE 9 and the portion, other than the alteration described in TABLE 8 or the one or more or combination of alterations described in TABLE 9, of the engineered ACL has at least 95% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and/or one or more or a combination of alterations described in TABLE 9 and the portion, other than the alteration described in TABLE 8 or the one or more or combination of alterations described in TABLE 9, of the engineered ACL has at least 98% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and/or one or more or a combination of alterations described in TABLE 9 and the portion, other than the alteration described in TABLE 8 or the one or more or combination of alterations described in TABLE 9, of the engineered ACL has at least 99% identical to SEQ ID NO: 1. In some embodiments, provided herein is microbial organism (e.g., host microbial organism) that has a recombinant nucleic acid encodes an engineered ACL that has an amino acid sequence that includes one or more alterations as described in TABLE 8 and/or one or more or a combination of alterations described in TABLE 9 and the portion, other than the alteration described in TABLE 8 or the one or more or combination of alterations described in TABLE 9, of the engineered ACL is identical to SEQ ID NO: 1.

In some embodiments, provided herein is a microbial organism, in particular a non-naturally occurring microbial organism, that expresses an engineered ACL described herein, that is, an engineered ACL described herein. Thus, provided herein is a non-naturally occurring microbial organism having a recombinant nucleic acid encoding an engineered ACL described herein. Accordingly, in some embodiments, provided herein is microbial organism (e.g., host microbial organism) that has a recombinant nucleic acid encoding an engineered ACL that is a variant of a wild-type or parent ACL (SEQ ID NO: 2), such as an engineered ACL having one or more alterations at a position described in TABLE 9, and/or one or more, or a combination of alternations described in TABLE 8.

In some embodiments, provided herein is microbial organism (e.g., host microbial organism) that has a recombinant nucleic acid encoding an engineered ACL that is a variant of SEQ ID NO: 2 that includes one or more alterations at a position described in TABLE 9, and wherein the engineered ACL further includes one or more or a combination of alterations described in TABLE 8. Accordingly, in some embodiments, the microbial organism has a recombinant nucleic acid that encodes an engineered ACL having: M at a residue corresponding to position 4 in SEQ ID NO: 2; D, K, Q, S, T at a residue corresponding to position 7 in SEQ ID NO: 2; I, S at a residue corresponding to position 8 in SEQ ID NO: 2; A, K, S at a residue corresponding to position 9 in SEQ ID NO: 2; F, H, P, V, W, Y at a residue corresponding to position 10 in SEQ ID NO: 2; D, E, H, P at a residue corresponding to position 12 in SEQ ID NO: 2; C, M at a residue corresponding to position 13 in SEQ ID NO: 2; D, I, L, P at a residue corresponding to position 15 in SEQ ID NO: 2; E, H, N at a residue corresponding to position 16 in SEQ ID NO: 2; F, L, P, Q at a residue corresponding to position 19 in SEQ ID NO: 2; W at a residue corresponding to position 20 in SEQ ID NO: 2; S at a residue corresponding to position 22 in SEQ ID NO: 2; D at a residue corresponding to position 30 in SEQ ID NO: 2; A at a residue corresponding to position 31 in SEQ ID NO: 2; G, P at a residue corresponding to position 35 in SEQ ID NO: 2; H at a residue corresponding to position 36 in SEQ ID NO: 2; A at a residue corresponding to position 39 in SEQ ID NO: 2; C, F, L, M at a residue corresponding to position 40 in SEQ ID NO: 2; G at a residue corresponding to position 43 in SEQ ID NO: 2; D, H, S, T at a residue corresponding to position 44 in SEQ ID NO: 2; R, RTYTQKEL (SEQ ID NO: 15), RTYTQREL (SEQ ID NO: 16), RTYT (SEQ ID NO: 17) at a residue corresponding to position 47 in SEQ ID NO: 2; a deletion at a residue corresponding to position 48 in SEQ ID NO: 2; a deletion, Y at a residue corresponding to position 49 in SEQ ID NO: 2; a deletion, T at a residue corresponding to position 50 in SEQ ID NO: 2; a deletion, F, H, L, R, W, Y at a residue corresponding to position 51 in SEQ ID NO: 2; a deletion, K, R at a residue corresponding to position 52 in SEQ ID NO: 2; a deletion at a residue corresponding to position 53 in SEQ ID NO: 2; a deletion, L at a residue corresponding to position 54 in SEQ ID NO: 2; L at a residue corresponding to position 55 in SEQ ID NO: 2; R at a residue corresponding to position 60 in SEQ ID NO: 2; F, L at a residue corresponding to position 61 in SEQ ID NO: 2; A, G at a residue corresponding to position 64 in SEQ ID NO: 2; R at a residue corresponding to position 66 in SEQ ID NO: 2; A at a residue corresponding to position 67 in SEQ ID NO: 2; L at a residue corresponding to position 69 in SEQ ID NO: 2; G, T at a residue corresponding to position 72 in SEQ ID NO: 2; F, G, L, V at a residue corresponding to position 73 in SEQ ID NO: 2; K at a residue corresponding to position 74 in SEQ ID NO: 2; T at a residue corresponding to position 76 in SEQ ID NO: 2; V at a residue corresponding to position 78 in SEQ ID NO: 2; G at a residue corresponding to position 80 in SEQ ID NO: 2; PG at a residue corresponding to position 81 in SEQ ID NO: 2; A, E, G, N, R, T at a residue corresponding to position 82 in SEQ ID NO: 2; a deletion, K, P, R, S at a residue corresponding to position 83 in SEQ ID NO: 2; I at a residue corresponding to position 86 in SEQ ID NO: 2; I, V, VAV at a residue corresponding to position 87 in SEQ ID NO: 2; a deletion, A at a residue corresponding to position 88 in SEQ ID NO: 2; a deletion, I, L, V at a residue corresponding to position 89 in SEQ ID NO: 2; C, L, M, V, W at a residue corresponding to position 90 in SEQ ID NO: 2; A, L, M, S at a residue corresponding to position 91 in SEQ ID NO: 2; C, CIEAA (SEQ ID NO: 18), S, T, TIEAA (SEQ ID NO: 19) at a residue corresponding to position 94 in SEQ ID NO: 2; a deletion at a residue corresponding to position 95 in SEQ ID NO: 2; a deletion, E at a residue corresponding to position 96 in SEQ ID NO: 2; a deletion at a residue corresponding to position 97 in SEQ ID NO: 2; a deletion, A, L at a residue corresponding to position 98 in SEQ ID NO: 2; C, M at a residue corresponding to position 99 in SEQ ID NO: 2; S, M, L at a residue corresponding to position 100 in SEQ ID NO: 2; F, M at a residue corresponding to position 101 in SEQ ID NO: 2; H, F, L at a residue corresponding to position 102 in SEQ ID NO: 2; G at a residue corresponding to position 103 in SEQ ID NO: 2; V, L, T at a residue corresponding to position 104 in SEQ ID NO: 2; V, W, H, A, I at a residue corresponding to position 105 in SEQ ID NO: 2; S, Y, R, K at a residue corresponding to position 106 in SEQ ID NO: 2; A, C, V at a residue corresponding to position 107 in SEQ ID NO: 2; L, V, Q at a residue corresponding to position 109 in SEQ ID NO: 2; V, A, M, L, C, VTPAN (SEQ ID NO: 20) at a residue corresponding to position 111 in SEQ ID NO: 2; T, a deletion at a residue corresponding to position 112 in SEQ ID NO: 2; a deletion, L at a residue corresponding to position 113 in SEQ ID NO: 2; a deletion, A, L, V at a residue corresponding to position 114 in SEQ ID NO: 2; a deletion, N at a residue corresponding to position 115 in SEQ ID NO: 2; A, W, P at a residue corresponding to position 116 in SEQ ID NO: 2; S at a residue corresponding to position 117 in SEQ ID NO: 2; T at a residue corresponding to position 119 in SEQ ID NO: 2; R, D at a residue corresponding to position 121 in SEQ ID NO: 2; H at a residue corresponding to position 126 in SEQ ID NO: 2; M, L, MRHTG (SEQ ID NO: 21), MRITG (SEQ ID NO: 22) at a residue corresponding to position 127 in SEQ ID NO: 2; a deletion at a residue corresponding to position 128 in SEQ ID NO: 2; a deletion, I, H at a residue corresponding to position 129 in SEQ ID NO: 2; a deletion at a residue corresponding to position 130 in SEQ ID NO: 2; a deletion, G, D at a residue corresponding to position 131 in SEQ ID NO: 2; L at a residue corresponding to position 136 in SEQ ID NO: 2; T at a residue corresponding to position 137 in SEQ ID NO: 2; D, S at a residue corresponding to position 138 in SEQ ID NO: 2; E at a residue corresponding to position 139 in SEQ ID NO: 2; E, K, D at a residue corresponding to position 140 in SEQ ID NO: 2; T at a residue corresponding to position 144 in SEQ ID NO: 2; R at a residue corresponding to position 149 in SEQ ID NO: 2; I, L at a residue corresponding to position 150 in SEQ ID NO: 2; A, T, C, I at a residue corresponding to position 152 in SEQ ID NO: 2; G, S at a residue corresponding to position 153 in SEQ ID NO: 2; G at a residue corresponding to position 159 in SEQ ID NO: 2; S, H, R, SIDAF (SEQ ID NO: 23), RIESF (SEQ ID NO: 24), HFDSF (SEQ ID NO: 25), HFDAF (SEQ ID NO: 26), HIDSF (SEQ ID NO: 27) at a residue corresponding to position 162 in SEQ ID NO: 2; F, I, a deletion at a residue corresponding to position 163 in SEQ ID NO: 2; a deletion, D at a residue corresponding to position 164 in SEQ ID NO: 2; a deletion, S at a residue corresponding to position 165 in SEQ ID NO: 2; a deletion, F at a residue corresponding to position 166 in SEQ ID NO: 2; L at a residue corresponding to position 167 in SEQ ID NO: 2; G at a residue corresponding to position 170 in SEQ ID NO: 2; L, A, D at a residue corresponding to position 172 in SEQ ID NO: 2; Q at a residue corresponding to position 182 in SEQ ID NO: 2; E, G at a residue corresponding to position 189 in SEQ ID NO: 2; A at a residue corresponding to position 191 in SEQ ID NO: 2; D at a residue corresponding to position 194 in SEQ ID NO: 2; T, P, M at a residue corresponding to position 195 in SEQ ID NO: 2; T, I at a residue corresponding to position 196 in SEQ ID NO: 2; W, C at a residue corresponding to position 197 in SEQ ID NO: 2; Y, V, S, W, I, M, T at a residue corresponding to position 198 in SEQ ID NO: 2; M, L, W, P, F, C, A at a residue corresponding to position 199 in SEQ ID NO: 2; W at a residue corresponding to position 200 in SEQ ID NO: 2; F, T, N, P, Y, L at a residue corresponding to position 201 in SEQ ID NO: 2; T, H, A, Q, E, L, G at a residue corresponding to position 202 in SEQ ID NO: 2; T at a residue corresponding to position 205 in SEQ ID NO: 2; F at a residue corresponding to position 208 in SEQ ID NO: 2; P at a residue corresponding to position 209 in SEQ ID NO: 2; G at a residue corresponding to position 211 in SEQ ID NO: 2; A, C at a residue corresponding to position 212 in SEQ ID NO: 2; M, V, E at a residue corresponding to position 213 in SEQ ID NO: 2; V at a residue corresponding to position 214 in SEQ ID NO: 2; S at a residue corresponding to position 215 in SEQ ID NO: 2; Q, R, Y, H, K, G at a residue corresponding to position 217 in SEQ ID NO: 2; W, M, V at a residue corresponding to position 220 in SEQ ID NO: 2; T, A at a residue corresponding to position 221 in SEQ ID NO: 2; S, N at a residue corresponding to position 222 in SEQ ID NO: 2; A, N, I, L, H at a residue corresponding to position 223 in SEQ ID NO: 2; T, Q, H, I, N, M at a residue corresponding to position 224 in SEQ ID NO: 2; L, A, F, Q at a residue corresponding to position 225 in SEQ ID NO: 2; Y, C, T, W at a residue corresponding to position 226 in SEQ ID NO: 2; G, E, M, T, A, GKWSQD (SEQ ID NO: 28), GKWSQSY (SEQ ID NO: 29), GKWSQ (SEQ ID NO: 30), GKWSQDLK (SEQ ID NO: 31), GKWSQQLQ (SEQ ID NO: 32) at a residue corresponding to position 227 in SEQ ID NO: 2; a deletion at a residue corresponding to position 228 in SEQ ID NO: 2; a deletion, F, S, L, G, W, I, M, H at a residue corresponding to position 229 in SEQ ID NO: 2; a deletion, R, M, P, T, E, Q, A at a residue corresponding to position 230 in SEQ ID NO: 2; a deletion, Q, P, H, QDLQ (SEQ ID NO: 33) at a residue corresponding to position 231 in SEQ ID NO: 2; a deletion, W, A, Y, N, K, Q, D at a residue corresponding to position 232 in SEQ ID NO: 2; a deletion, W, R, M, Y, P at a residue corresponding to position 233 in SEQ ID NO: 2; a deletion, F, Y, T, K, R, Q, P, G, H, E at a residue corresponding to position 234 in SEQ ID NO: 2; H, S, R, K, M, Q, D, G at a residue corresponding to position 235 in SEQ ID NO: 2; P, N, H, T, D, DISAC (SEQ ID NO: 34), DVS at a residue corresponding to position 238 in SEQ ID NO: 2; V, F, R, a deletion at a residue corresponding to position 239 in SEQ ID NO: 2; a deletion, F, H, T, W, S, L, C, V at a residue corresponding to position 240 in SEQ ID NO: 2; a deletion, C, M, P, I at a residue corresponding to position 241 in SEQ ID NO: 2; a deletion, G, V, C at a residue corresponding to position 242 in SEQ ID NO: 2; L, C, M at a residue corresponding to position 243 in SEQ ID NO: 2; M, V at a residue corresponding to position 244 in SEQ ID NO: 2; W, F, L, Y at a residue corresponding to position 246 in SEQ ID NO: 2; F, C at a residue corresponding to position 247 in SEQ ID NO: 2; S, P at a residue corresponding to position 248 in SEQ ID NO: 2; A, V, M at a residue corresponding to position 249 in SEQ ID NO: 2; M, A, W at a residue corresponding to position 250 in SEQ ID NO: 2; A, H at a residue corresponding to position 251 in SEQ ID NO: 2; I, M, W, Q at a residue corresponding to position 252 in SEQ ID NO: 2; T, Q, N, I, H, V, M at a residue corresponding to position 253 in SEQ ID NO: 2; E, I, T, Y, H, V, L, P, A, M, F, S, G at a residue corresponding to position 254 in SEQ ID NO: 2; M, T, I, N, Y, C, ILLANV (SEQ ID NO: 35), ILLANVHY (SEQ ID NO: 36), ILLLNVHY (SEQ ID NO: 37), ILLLNV (SEQ ID NO: 38), ILLLNVHK (SEQ ID NO: 39), ILLHNVHK (SEQ ID NO: 40), ILLSNVHY (SEQ ID NO: 41) at a residue corresponding to position 255 in SEQ ID NO: 2; I, V, H, C, F, a deletion at a residue corresponding to position 256 in SEQ ID NO: 2; a deletion, L, T, N at a residue corresponding to position 257 in SEQ ID NO: 2; a deletion, S, L, P, M, H, T, A at a residue corresponding to position 258 in SEQ ID NO: 2; a deletion, C, A at a residue corresponding to position 259 in SEQ ID NO: 2; a deletion, F, L, M, V at a residue corresponding to position 260 in SEQ ID NO: 2; a deletion, Y, M, W, A, F, L, S at a residue corresponding to position 261 in SEQ ID NO: 2; a deletion, M, K, Y, R, G, C, L, V, S, N at a residue corresponding to position 262 in SEQ ID NO: 2; G, W, I, S, V at a residue corresponding to position 264 in SEQ ID NO: 2; R, C, H, K at a residue corresponding to position 265 in SEQ ID NO: 2; I, V, Q, C at a residue corresponding to position 266 in SEQ ID NO: 2; Y at a residue corresponding to position 267 in SEQ ID NO: 2; V, M, T, F, IMPR (SEQ ID NO: 42), IMNR (SEQ ID NO: 43) at a residue corresponding to position 268 in SEQ ID NO: 2; M, at a residue corresponding to position 269 in SEQ ID NO: 2; a deletion, P at a residue corresponding to position 270 in SEQ ID NO: 2; a deletion, R, Q at a residue corresponding to position 271 in SEQ ID NO: 2; W at a residue corresponding to position 272 in SEQ ID NO: 2; P, PEEMLRAIQ (SEQ ID NO: 44), PEEMLQAIQ (SEQ ID NO: 45) at a residue corresponding to position 274 in SEQ ID NO: 2; D, A, E, R, K, EEMLQAIQ (SEQ ID NO: 46), EEMLRAIQ (SEQ ID NO: 47), a deletion, KEMLQAIQ (SEQ ID NO: 48) at a residue corresponding to position 275 in SEQ ID NO: 2; a deletion at a residue corresponding to position 276 in SEQ ID NO: 2; a deletion, M, Y, L, F at a residue corresponding to position 277 in SEQ ID NO: 2; a deletion at a residue corresponding to position 278 in SEQ ID NO: 2; a deletion, Q, R, QAIQ (SEQ ID NO: 49), RAIQ (SEQ ID NO: 50) at a residue corresponding to position 279 in SEQ ID NO: 2; a deletion at a residue corresponding to position 280 in SEQ ID NO: 2; a deletion, V, M, L, I at a residue corresponding to position 281 in SEQ ID NO: 2; a deletion, Q at a residue corresponding to position 282 in SEQ ID NO: 2; P, C at a residue corresponding to position 286 in SEQ ID NO: 2; T, TVFFA (SEQ ID NO: 51), TSFFA (SEQ ID NO: 52), TFFFA (SEQ ID NO: 53), TMFFA (SEQ ID NO: 54), TVFHA (SEQ ID NO: 55) at a residue corresponding to position 287 in SEQ ID NO: 2; H, F, M, Y, R, I, V, T, C, a deletion at a residue corresponding to position 288 in SEQ ID NO: 2; a deletion, M, V, A, L, F, I at a residue corresponding to position 289 in SEQ ID NO: 2; a deletion, A, W, H, C, F, V, P, L at a residue corresponding to position 290 in SEQ ID NO: 2; a deletion, A, M, G, V at a residue corresponding to position 291 in SEQ ID NO: 2; A, T at a residue corresponding to position 294 in SEQ ID NO: 2; L, a deletion at a residue corresponding to position 295 in SEQ ID NO: 2; M, L at a residue corresponding to position 296 in SEQ ID NO: 2; M, Q, I, A, L, T at a residue corresponding to position 297 in SEQ ID NO: 2; M, A, F, R, IM at a residue corresponding to position 298 in SEQ ID NO: 2; M, F at a residue corresponding to position 299 in SEQ ID NO: 2; A at a residue corresponding to position 300 in SEQ ID NO: 2; M, R, E at a residue corresponding to position 304 in SEQ ID NO: 2; D at a residue corresponding to position 306 in SEQ ID NO: 2; L at a residue corresponding to position 309 in SEQ ID NO: 2; L, D at a residue corresponding to position 310 in SEQ ID NO: 2; L at a residue corresponding to position 311 in SEQ ID NO: 2; S, SHVRSV (SEQ ID NO: 56), SHVRTV (SEQ ID NO: 57) at a residue corresponding to position 314 in SEQ ID NO: 2; S, a deletion at a residue corresponding to position 315 in SEQ ID NO: 2; a deletion, V, L, I at a residue corresponding to position 316 in SEQ ID NO: 2; a deletion, H, N, Q, T at a residue corresponding to position 317 in SEQ ID NO: 2; a deletion, Y, M, T, S, C, Q, R, H, V, W, I, L at a residue corresponding to position 318 in SEQ ID NO: 2; a deletion, C, F, V, L, M at a residue corresponding to position 319 in SEQ ID NO: 2; F, I, V, Y, T, M, W at a residue corresponding to position 320 in SEQ ID NO: 2; C at a residue corresponding to position 321 in SEQ ID NO: 2; G at a residue corresponding to position 323 in SEQ ID NO: 2; S, M at a residue corresponding to position 324 in SEQ ID NO: 2; V, I, C at a residue corresponding to position 326 in SEQ ID NO: 2; S at a residue corresponding to position 327 in SEQ ID NO: 2; H, V, R, S, RELE (SEQ ID NO: 58) at a residue corresponding to position 328 in SEQ ID NO: 2; E, a deletion, ELE at a residue corresponding to position 329 in SEQ ID NO: 2; a deletion, L, T, I at a residue corresponding to position 330 in SEQ ID NO: 2; a deletion, M, E, C, L, I at a residue corresponding to position 331 in SEQ ID NO: 2; A, W, C, M, I at a residue corresponding to position 334 in SEQ ID NO: 2; Q, R at a residue corresponding to position 335 in SEQ ID NO: 2; K at a residue corresponding to position 336 in SEQ ID NO: 2; M at a residue corresponding to position 338 in SEQ ID NO: 2; S at a residue corresponding to position 339 in SEQ ID NO: 2; H, T at a residue corresponding to position 341 in SEQ ID NO: 2; L, F at a residue corresponding to position 342 in SEQ ID NO: 2; D, H, Y at a residue corresponding to position 348 in SEQ ID NO: 2; V, C, T, F, A, W at a residue corresponding to position 351 in SEQ ID NO: 2; E, K, D at a residue corresponding to position 352 in SEQ ID NO: 2; V, L, M at a residue corresponding to position 353 in SEQ ID NO: 2; C, F, L, Y, A, K, S, I at a residue corresponding to position 354 in SEQ ID NO: 2; E at a residue corresponding to position 355 in SEQ ID NO: 2; A at a residue corresponding to position 356 in SEQ ID NO: 2; W at a residue corresponding to position 357 in SEQ ID NO: 2; M at a residue corresponding to position 359 in SEQ ID NO: 2; S at a residue corresponding to position 360 in SEQ ID NO: 2; C, S at a residue corresponding to position 362 in SEQ ID NO: 2; A, T, G at a residue corresponding to position 363 in SEQ ID NO: 2; C, S at a residue corresponding to position 364 in SEQ ID NO: 2; L, I, M, C at a residue corresponding to position 365 in SEQ ID NO: 2; S, V, T at a residue corresponding to position 366 in SEQ ID NO: 2; A, C, S, H at a residue corresponding to position 367 in SEQ ID NO: 2; H at a residue corresponding to position 368 in SEQ ID NO: 2; H, Y, I, C, L, V, W, S, T, M at a residue corresponding to position 369 in SEQ ID NO: 2; Y, R, T, S, H, G, N, Q at a residue corresponding to position 370 in SEQ ID NO: 2; W at a residue corresponding to position 371 in SEQ ID NO: 2; K, W, Q, H, S, R, N at a residue corresponding to position 372 in SEQ ID NO: 2; S, H, P, Q, E at a residue corresponding to position 373 in SEQ ID NO: 2; D, T, A, R, S, P, W, E, Q, H at a residue corresponding to position 374 in SEQ ID NO: 2; P at a residue corresponding to position 375 in SEQ ID NO: 2; T, K, E, Q, H, D at a residue corresponding to position 376 in SEQ ID NO: 2; Y, R, N at a residue corresponding to position 377 in SEQ ID NO: 2; S, Y, P, V, A at a residue corresponding to position 378 in SEQ ID NO: 2; G at a residue corresponding to position 379 in SEQ ID NO: 2; T at a residue corresponding to position 380 in SEQ ID NO: 2; V, S, I at a residue corresponding to position 381 in SEQ ID NO: 2; Q, H, T, Y, W, P at a residue corresponding to position 383 in SEQ ID NO: 2; I, P, A, M at a residue corresponding to position 384 in SEQ ID NO: 2; S, A at a residue corresponding to position 386 in SEQ ID NO: 2; G, S, H at a residue corresponding to position 387 in SEQ ID NO: 2; T, M, V, L at a residue corresponding to position 388 in SEQ ID NO: 2; R, K, E at a residue corresponding to position 389 in SEQ ID NO: 2; C, L, A at a residue corresponding to position 390 in SEQ ID NO: 2; K at a residue corresponding to position 391 in SEQ ID NO: 2; D at a residue corresponding to position 394 in SEQ ID NO: 2; D, N at a residue corresponding to position 398 in SEQ ID NO: 2; V, L, I at a residue corresponding to position 402 in SEQ ID NO: 2; L, V at a residue corresponding to position 406 in SEQ ID NO: 2; D at a residue corresponding to position 407 in SEQ ID NO: 2; N, Q, NTENAPR (SEQ ID NO: 59), NTENAPD (SEQ ID NO: 60) at a residue corresponding to position 410 in SEQ ID NO: 2; T, TENAPD (SEQ ID NO: 61), TQNAPR (SEQ ID NO: 62), a deletion, TENAPR (SEQ ID NO: 63), TQNAPN (SEQ ID NO: 64) at a residue corresponding to position 411 in SEQ ID NO: 2; a deletion, Q at a residue corresponding to position 412 in SEQ ID NO: 2; a deletion, N, NNPN (SEQ ID NO: 65) at a residue corresponding to position 413 in SEQ ID NO: 2; a deletion, A, S, N at a residue corresponding to position 414 in SEQ ID NO: 2; a deletion, P at a residue corresponding to position 415 in SEQ ID NO: 2; a deletion, D, R, N, E at a residue corresponding to position 416 in SEQ ID NO: 2; P at a residue corresponding to position 418 in SEQ ID NO: 2; G at a residue corresponding to position 419 in SEQ ID NO: 2; E at a residue corresponding to position 420 in SEQ ID NO: 2; M, V at a residue corresponding to position 424 in SEQ ID NO: 2; V, L at a residue corresponding to position 425 in SEQ ID NO: 2; M, L at a residue corresponding to position 426 in SEQ ID NO: 2; T, N, S at a residue corresponding to position 430 in SEQ ID NO: 2; I, L at a residue corresponding to position 431 in SEQ ID NO: 2; T, F at a residue corresponding to position 432 in SEQ ID NO: 2; S, K at a residue corresponding to position 433 in SEQ ID NO: 2; W at a residue corresponding to position 436 in SEQ ID NO: 2; T at a residue corresponding to position 442 in SEQ ID NO: 2; R, K, RASFA (SEQ ID NO: 66) at a residue corresponding to position 443 in SEQ ID NO: 2; a deletion at a residue corresponding to position 444 in SEQ ID NO: 2; a deletion, M, T, S, A at a residue corresponding to position 445 in SEQ ID NO: 2; a deletion at a residue corresponding to position 446 in SEQ ID NO: 2; a deletion, A at a residue corresponding to position 447 in SEQ ID NO: 2; S, G at a residue corresponding to position 455 in SEQ ID NO: 2; G, E, D at a residue corresponding to position 459 in SEQ ID NO: 2; D, G at a residue corresponding to position 461 in SEQ ID NO: 2; R, G at a residue corresponding to position 462 in SEQ ID NO: 2; L, Y at a residue corresponding to position 464 in SEQ ID NO: 2; H at a residue corresponding to position 465 in SEQ ID NO: 2; M at a residue corresponding to position 469 in SEQ ID NO: 2; C, G at a residue corresponding to position 470 in SEQ ID NO: 2; Y, K, M, W, I, R at a residue corresponding to position 471 in SEQ ID NO: 2; I, F, C, Y, L, M at a residue corresponding to position 472 in SEQ ID NO: 2; E, EDGH (SEQ ID NO: 67), EDGY (SEQ ID NO: 68) at a residue corresponding to position 474 in SEQ ID NO: 2; D, a deletion at a residue corresponding to position 475 in SEQ ID NO: 2; a deletion at a residue corresponding to position 476 in SEQ ID NO: 2; a deletion, H, F, N, E, Y at a residue corresponding to position 477 in SEQ ID NO: 2; W, I, M at a residue corresponding to position 478 in SEQ ID NO: 2; T, H, Y, F, W at a residue corresponding to position 479 in SEQ ID NO: 2; V, L at a residue corresponding to position 480 in SEQ ID NO: 2; C, I, T at a residue corresponding to position 481 in SEQ ID NO: 2; L at a residue corresponding to position 484 in SEQ ID NO: 2; D at a residue corresponding to position 486 in SEQ ID NO: 2; I at a residue corresponding to position 489 in SEQ ID NO: 2; R, C, V at a residue corresponding to position 490 in SEQ ID NO: 2; G, R, N at a residue corresponding to position 491 in SEQ ID NO: 2; F, H, Y, L at a residue corresponding to position 493 in SEQ ID NO: 2; N at a residue corresponding to position 494 in SEQ ID NO: 2; F, S, Y at a residue corresponding to position 496 in SEQ ID NO: 2; L, V, I at a residue corresponding to position 503 in SEQ ID NO: 2; R, RHPD (SEQ ID NO: 69) at a residue corresponding to position 506 in SEQ ID NO: 2; a deletion at a residue corresponding to position 507 in SEQ ID NO: 2; a deletion, P at a residue corresponding to position 508 in SEQ ID NO: 2; a deletion, D at a residue corresponding to position 509 in SEQ ID NO: 2; A, V, C at a residue corresponding to position 513 in SEQ ID NO: 2; V at a residue corresponding to position 518 in SEQ ID NO: 2; P at a residue corresponding to position 519 in SEQ ID NO: 2; E, P at a residue corresponding to position 521 in SEQ ID NO: 2; Q, A, R, M, Y, D at a residue corresponding to position 523 in SEQ ID NO: 2; S, A at a residue corresponding to position 524 in SEQ ID NO: 2; S, T, D, G, E at a residue corresponding to position 525 in SEQ ID NO: 2; P, T, V at a residue corresponding to position 527 in SEQ ID NO: 2; A, V at a residue corresponding to position 528 in SEQ ID NO: 2; V, A at a residue corresponding to position 529 in SEQ ID NO: 2; L, V at a residue corresponding to position 539 in SEQ ID NO: 2; T at a residue corresponding to position 540 in SEQ ID NO: 2; E, D at a residue corresponding to position 542 in SEQ ID NO: 2; A, K at a residue corresponding to position 546 in SEQ ID NO: 2; K, R at a residue corresponding to position 551 in SEQ ID NO: 2; S at a residue corresponding to position 553 in SEQ ID NO: 2; S, P, H at a residue corresponding to position 554 in SEQ ID NO: 2; F at a residue corresponding to position 555 in SEQ ID NO: 2; K at a residue corresponding to position 557 in SEQ ID NO: 2; P at a residue corresponding to position 559 in SEQ ID NO: 2; G at a residue corresponding to position 560 in SEQ ID NO: 2; V, a deletion at a residue corresponding to position 562 in SEQ ID NO: 2; F at a residue corresponding to position 564 in SEQ ID NO: 2; V, F, FV at a residue corresponding to position 565 in SEQ ID NO: 2; E, VE at a residue corresponding to position 566 in SEQ ID NO: 2; Q, E at a residue corresponding to position 583 in SEQ ID NO: 2; R, E at a residue corresponding to position 5 in SEQ ID NO: 2; L at a residue corresponding to position 6 in SEQ ID NO: 2; K, T at a residue corresponding to position 7 in SEQ ID NO: 2; P at a residue corresponding to position 8 in SEQ ID NO: 2; K, D, P, N, R at a residue corresponding to position 10 in SEQ ID NO: 2; A, I, V, M at a residue corresponding to position 12 in SEQ ID NO: 2; C at a residue corresponding to position 13 in SEQ ID NO: 2; P, A, Q at a residue corresponding to position 14 in SEQ ID NO: 2; E, L, S, R at a residue corresponding to position 15 in SEQ ID NO: 2; C, L, I at a residue corresponding to position 16 in SEQ ID NO: 2; D, R at a residue corresponding to position 19 in SEQ ID NO: 2; H, K, R, D at a residue corresponding to position 20 in SEQ ID NO: 2; D, E at a residue corresponding to position 22 in SEQ ID NO: 2; N at a residue corresponding to position 25 in SEQ ID NO: 2; M at a residue corresponding to position 28 in SEQ ID NO: 2; C at a residue corresponding to position 37 in SEQ ID NO: 2; R at a residue corresponding to position 39 in SEQ ID NO: 2; T at a residue corresponding to position 40 in SEQ ID NO: 2; M, L, V, I at a residue corresponding to position 41 in SEQ ID NO: 2; S at a residue corresponding to position 43 in SEQ ID NO: 2; M at a residue corresponding to position 44 in SEQ ID NO: 2; I, V at a residue corresponding to position 45 in SEQ ID NO: 2; P at a residue corresponding to position 48 in SEQ ID NO: 2; I, F, R, V, L at a residue corresponding to position 50 in SEQ ID NO: 2; R at a residue corresponding to position 51 in SEQ ID NO: 2; L at a residue corresponding to position 53 in SEQ ID NO: 2; F at a residue corresponding to position 57 in SEQ ID NO: 2; V, L, M, F at a residue corresponding to position 58 in SEQ ID NO: 2; V at a residue corresponding to position 59 in SEQ ID NO: 2; P, E at a residue corresponding to position 61 in SEQ ID NO: 2; D at a residue corresponding to position 62 in SEQ ID NO: 2; D at a residue corresponding to position 63 in SEQ ID NO: 2; G at a residue corresponding to position 66 in SEQ ID NO: 2; K at a residue corresponding to position 67 in SEQ ID NO: 2; F, Y, L at a residue corresponding to position 69 in SEQ ID NO: 2; F, W at a residue corresponding to position 71 in SEQ ID NO: 2; A, R at a residue corresponding to position 72 in SEQ ID NO: 2; F at a residue corresponding to position 74 in SEQ ID NO: 2; D at a residue corresponding to position 75 in SEQ ID NO: 2; T, D, A, R at a residue corresponding to position 76 in SEQ ID NO: 2; R at a residue corresponding to position 77 in SEQ ID NO: 2; A at a residue corresponding to position 78 in SEQ ID NO: 2; K at a residue corresponding to position 80 in SEQ ID NO: 2; C at a residue corresponding to position 81 in SEQ ID NO: 2; S, H, Q, N at a residue corresponding to position 83 in SEQ ID NO: 2; Y, H at a residue corresponding to position 84 in SEQ ID NO: 2; F at a residue corresponding to position 85 in SEQ ID NO: 2; A, Q at a residue corresponding to position 86 in SEQ ID NO: 2; R at a residue corresponding to position 87 in SEQ ID NO: 2; L at a residue corresponding to position 88 in SEQ ID NO: 2; K at a residue corresponding to position 92 in SEQ ID NO: 2; G at a residue corresponding to position 93 in SEQ ID NO: 2; R at a residue corresponding to position 94 in SEQ ID NO: 2; R at a residue corresponding to position 95 in SEQ ID NO: 2; A at a residue corresponding to position 97 in SEQ ID NO: 2; M at a residue corresponding to position 98 in SEQ ID NO: 2; L at a residue corresponding to position 99 in SEQ ID NO: 2; C at a residue corresponding to position 100 in SEQ ID NO: 2; P at a residue corresponding to position 101 in SEQ ID NO: 2; R at a residue corresponding to position 103 in SEQ ID NO: 2; L, W, A at a residue corresponding to position 104 in SEQ ID NO: 2; W, F at a residue corresponding to position 106 in SEQ ID NO: 2; V, P, I, L, W, F at a residue corresponding to position 107 in SEQ ID NO: 2; V at a residue corresponding to position 108 in SEQ ID NO: 2; V at a residue corresponding to position 109 in SEQ ID NO: 2; M, F, Y, A, D at a residue corresponding to position 110 in SEQ ID NO: 2; T, C, V at a residue corresponding to position 113 in SEQ ID NO: 2; M, W, L, G, A at a residue corresponding to position 114 in SEQ ID NO: 2; A at a residue corresponding to position 115 in SEQ ID NO: 2; I at a residue corresponding to position 116 in SEQ ID NO: 2; V, T, I at a residue corresponding to position 119 in SEQ ID NO: 2; M, F, V, A, L, W, Y at a residue corresponding to position 120 in SEQ ID NO: 2; T at a residue corresponding to position 121 in SEQ ID NO: 2; A at a residue corresponding to position 122 in SEQ ID NO: 2; M, V, I at a residue corresponding to position 123 in SEQ ID NO: 2; Y, S, N at a residue corresponding to position 124 in SEQ ID NO: 2; P, T, W at a residue corresponding to position 125 in SEQ ID NO: 2; M, N, R, A at a residue corresponding to position 126 in SEQ ID NO: 2; F, W, L at a residue corresponding to position 127 in SEQ ID NO: 2; R at a residue corresponding to position 128 in SEQ ID NO: 2; M at a residue corresponding to position 130 in SEQ ID NO: 2; E at a residue corresponding to position 131 in SEQ ID NO: 2; E, Q, K at a residue corresponding to position 136 in SEQ ID NO: 2; M at a residue corresponding to position 137 in SEQ ID NO: 2; M, C, I at a residue corresponding to position 140 in SEQ ID NO: 2; M at a residue corresponding to position 141 in SEQ ID NO: 2; N, Q at a residue corresponding to position 142 in SEQ ID NO: 2; G, R, D at a residue corresponding to position 145 in SEQ ID NO: 2; S at a residue corresponding to position 147 in SEQ ID NO: 2; L, V, A at a residue corresponding to position 148 in SEQ ID NO: 2; V, L at a residue corresponding to position 149 in SEQ ID NO: 2; V at a residue corresponding to position 150 in SEQ ID NO: 2; C at a residue corresponding to position 151 in SEQ ID NO: 2; L at a residue corresponding to position 157 in SEQ ID NO: 2; R, E at a residue corresponding to position 159 in SEQ ID NO: 2; I at a residue corresponding to position 161 in SEQ ID NO: 2; V at a residue corresponding to position 163 in SEQ ID NO: 2; A at a residue corresponding to position 164 in SEQ ID NO: 2; R at a residue corresponding to position 165 in SEQ ID NO: 2; R at a residue corresponding to position 167 in SEQ ID NO: 2; H at a residue corresponding to position 168 in SEQ ID NO: 2; I at a residue corresponding to position 169 in SEQ ID NO: 2; I, V, L at a residue corresponding to position 170 in SEQ ID NO: 2; V at a residue corresponding to position 173 in SEQ ID NO: 2; R at a residue corresponding to position 175 in SEQ ID NO: 2; D at a residue corresponding to position 176 in SEQ ID NO: 2; E at a residue corresponding to position 177 in SEQ ID NO: 2; E at a residue corresponding to position 181 in SEQ ID NO: 2; I at a residue corresponding to position 183 in SEQ ID NO: 2; S at a residue corresponding to position 185 in SEQ ID NO: 2; Y at a residue corresponding to position 186 in SEQ ID NO: 2; D at a residue corresponding to position 187 in SEQ ID NO: 2; V at a residue corresponding to position 189 in SEQ ID NO: 2; V at a residue corresponding to position 190 in SEQ ID NO: 2; A, E at a residue corresponding to position 191 in SEQ ID NO: 2; T at a residue corresponding to position 192 in SEQ ID NO: 2; A at a residue corresponding to position 193 in SEQ ID NO: 2; S at a residue corresponding to position 194 in SEQ ID NO: 2; S at a residue corresponding to position 195 in SEQ ID NO: 2; P at a residue corresponding to position 196 in SEQ ID NO: 2; A, P, L at a residue corresponding to position 201 in SEQ ID NO: 2; T at a residue corresponding to position 205 in SEQ ID NO: 2; D at a residue corresponding to position 208 in SEQ ID NO: 2; I, T, P, V at a residue corresponding to position 209 in SEQ ID NO: 2; L, T, C, M, V, A at a residue corresponding to position 211 in SEQ ID NO: 2; L, M at a residue corresponding to position 212 in SEQ ID NO: 2; I, C, V, Q, M at a residue corresponding to position 213 in SEQ ID NO: 2; Y at a residue corresponding to position 214 in SEQ ID NO: 2; S at a residue corresponding to position 215 in SEQ ID NO: 2; G at a residue corresponding to position 216 in SEQ ID NO: 2; T at a residue corresponding to position 218 in SEQ ID NO: 2; T at a residue corresponding to position 219 in SEQ ID NO: 2; G at a residue corresponding to position 220 in SEQ ID NO: 2; T, M, L, R, K, H at a residue corresponding to position 221 in SEQ ID NO: 2; S at a residue corresponding to position 222 in SEQ ID NO: 2; A at a residue corresponding to position 224 in SEQ ID NO: 2; A at a residue corresponding to position 225 in SEQ ID NO: 2; M, I, V at a residue corresponding to position 226 in SEQ ID NO: 2; L, D, H, I at a residue corresponding to position 227 in SEQ ID NO: 2; S at a residue corresponding to position 228 in SEQ ID NO: 2; H, K at a residue corresponding to position 230 in SEQ ID NO: 2; N at a residue corresponding to position 231 in SEQ ID NO: 2; L, I, V at a residue corresponding to position 232 in SEQ ID NO: 2; L, V, M, W, A, I at a residue corresponding to position 233 in SEQ ID NO: 2; S, W at a residue corresponding to position 234 in SEQ ID NO: 2; Q, S at a residue corresponding to position 235 in SEQ ID NO: 2; A, C, I, M, V at a residue corresponding to position 236 in SEQ ID NO: 2; A, M, Q, R, E at a residue corresponding to position 237 in SEQ ID NO: 2; M, S, W, N, H at a residue corresponding to position 238 in SEQ ID NO: 2; F, M, V, C, L at a residue corresponding to position 239 in SEQ ID NO: 2; A, R at a residue corresponding to position 240 in SEQ ID NO: 2; A, H, E, Q at a residue corresponding to position 241 in SEQ ID NO: 2; V, C, T, W at a residue corresponding to position 242 in SEQ ID NO: 2; I, F, M, W, V at a residue corresponding to position 243 in SEQ ID NO: 2; E, P, Q, H, G at a residue corresponding to position 244 in SEQ ID NO: 2; M, L, H, W, Y at a residue corresponding to position 245 in SEQ ID NO: 2; I, T, M at a residue corresponding to position 246 in SEQ ID NO: 2; R, P, E at a residue corresponding to position 247 in SEQ ID NO: 2; E at a residue corresponding to position 251 in SEQ ID NO: 2; H, V at a residue corresponding to position 252 in SEQ ID NO: 2; Y, M, F, L, V at a residue corresponding to position 253 in SEQ ID NO: 2; I, F, V, M at a residue corresponding to position 254 in SEQ ID NO: 2; A, T, N, L, I, G, H, C, V, S at a residue corresponding to position 255 in SEQ ID NO: 2; F, V, I, C, Y at a residue corresponding to position 256 in SEQ ID NO: 2; M at a residue corresponding to position 257 in SEQ ID NO: 2; L, F at a residue corresponding to position 259 in SEQ ID NO: 2; H, S, Y, F at a residue corresponding to position 260 in SEQ ID NO: 2; C, T, A, I, M at a residue corresponding to position 262 in SEQ ID NO: 2; A, Y, H at a residue corresponding to position 263 in SEQ ID NO: 2; A at a residue corresponding to position 264 in SEQ ID NO: 2; M, L, W, F, C at a residue corresponding to position 265 in SEQ ID NO: 2; N, T, M, V at a residue corresponding to position 266 in SEQ ID NO: 2; I, G, M, W, F, L, V, C, A at a residue corresponding to position 267 in SEQ ID NO: 2; M, A, C, G, V at a residue corresponding to position 268 in SEQ ID NO: 2; L, M, I, V at a residue corresponding to position 269 in SEQ ID NO: 2; M, H, Y, T at a residue corresponding to position 270 in SEQ ID NO: 2; A, P, C, T, L at a residue corresponding to position 271 in SEQ ID NO: 2; C, V, P, A, T at a residue corresponding to position 272 in SEQ ID NO: 2; M, V, F, I at a residue corresponding to position 273 in SEQ ID NO: 2; Y, R, V, M, C, H, W at a residue corresponding to position 274 in SEQ ID NO: 2; G, C, S, H at a residue corresponding to position 275 in SEQ ID NO: 2; A, C at a residue corresponding to position 277 in SEQ ID NO: 2; C, T, A, H at a residue corresponding to position 278 in SEQ ID NO: 2; V, M, I at a residue corresponding to position 279 in SEQ ID NO: 2; F, L, V, M at a residue corresponding to position 281 in SEQ ID NO: 2; P, M, E, A, H at a residue corresponding to position 282 in SEQ ID NO: 2; E, D, P, R at a residue corresponding to position 283 in SEQ ID NO: 2; F, R, W at a residue corresponding to position 284 in SEQ ID NO: 2; R, F, W, D at a residue corresponding to position 285 in SEQ ID NO: 2; D, F at a residue corresponding to position 286 in SEQ ID NO: 2; P, A, V at a residue corresponding to position 287 in SEQ ID NO: 2; A at a residue corresponding to position 290 in SEQ ID NO: 2; M, V, L, P, I at a residue corresponding to position 291 in SEQ ID NO: 2; H at a residue corresponding to position 292 in SEQ ID NO: 2; I, L, A at a residue corresponding to position 294 in SEQ ID NO: 2; W, M, V, I at a residue corresponding to position 295 in SEQ ID NO: 2; D, Q at a residue corresponding to position 296 in SEQ ID NO: 2; M, V, L at a residue corresponding to position 298 in SEQ ID NO: 2; A at a residue corresponding to position 299 in SEQ ID NO: 2; Q at a residue corresponding to position 300 in SEQ ID NO: 2; Q at a residue corresponding to position 302 in SEQ ID NO: 2; V, I, A, P, C at a residue corresponding to position 303 in SEQ ID NO: 2; S at a residue corresponding to position 304 in SEQ ID NO: 2; W, Y, V, I, F, H, M at a residue corresponding to position 305 in SEQ ID NO: 2; V, Y, I, M, L, F at a residue corresponding to position 306 in SEQ ID NO: 2; P, W, Y, H, M at a residue corresponding to position 307 in SEQ ID NO: 2; T, A, S, L, G, V, M at a residue corresponding to position 308 in SEQ ID NO: 2; A, R, P, T at a residue corresponding to position 311 in SEQ ID NO: 2; I, M, V, F at a residue corresponding to position 312 in SEQ ID NO: 2; L, F, W at a residue corresponding to position 313 in SEQ ID NO: 2; Q, E, H at a residue corresponding to position 314 in SEQ ID NO: 2; R, A, K, L at a residue corresponding to position 315 in SEQ ID NO: 2; W, M, I, F at a residue corresponding to position 316 in SEQ ID NO: 2; H, D at a residue corresponding to position 318 in SEQ ID NO: 2; R, Q at a residue corresponding to position 321 in SEQ ID NO: 2; Q, K, A, H at a residue corresponding to position 322 in SEQ ID NO: 2; L, F at a residue corresponding to position 330 in SEQ ID NO: 2; G at a residue corresponding to position 331 in SEQ ID NO: 2; S at a residue corresponding to position 333 in SEQ ID NO: 2; I, M, V at a residue corresponding to position 334 in SEQ ID NO: 2; R, K at a residue corresponding to position 335 in SEQ ID NO: 2; T, I, M, Y, V, F, W at a residue corresponding to position 336 in SEQ ID NO: 2; L, M, F, A, V at a residue corresponding to position 337 in SEQ ID NO: 2; W, L, M, V at a residue corresponding to position 338 in SEQ ID NO: 2; F, T, S, C at a residue corresponding to position 339 in SEQ ID NO: 2; G at a residue corresponding to position 340 in SEQ ID NO: 2; A, S at a residue corresponding to position 341 in SEQ ID NO: 2; S at a residue corresponding to position 342 in SEQ ID NO: 2; P, A at a residue corresponding to position 343 in SEQ ID NO: 2; M, I, V, C at a residue corresponding to position 344 in SEQ ID NO: 2; S at a residue corresponding to position 345 in SEQ ID NO: 2; V, A, P, E at a residue corresponding to position 346 in SEQ ID NO: 2; E at a residue corresponding to position 347 in SEQ ID NO: 2; T, I, L at a residue corresponding to position 348 in SEQ ID NO: 2; M, F, L, A, I, Y at a residue corresponding to position 349 in SEQ ID NO: 2; F, R, Q, W at a residue corresponding to position 351 in SEQ ID NO: 2; I, M, Y at a residue corresponding to position 352 in SEQ ID NO: 2; R at a residue corresponding to position 353 in SEQ ID NO: 2; K at a residue corresponding to position 354 in SEQ ID NO: 2; A, R at a residue corresponding to position 355 in SEQ ID NO: 2; W, G, V, L, P, I, F, A at a residue corresponding to position 357 in SEQ ID NO: 2; Q at a residue corresponding to position 358 in SEQ ID NO: 2; H, Q at a residue corresponding to position 359 in SEQ ID NO: 2; F at a residue corresponding to position 361 in SEQ ID NO: 2; H, L, C at a residue corresponding to position 362 in SEQ ID NO: 2; D, V, I, G at a residue corresponding to position 363 in SEQ ID NO: 2; M, V, I at a residue corresponding to position 365 in SEQ ID NO: 2; I, V, L, F at a residue corresponding to position 366 in SEQ ID NO: 2; T, C, M, I, V, L at a residue corresponding to position 367 in SEQ ID NO: 2; N, Q, E, T at a residue corresponding to position 368 in SEQ ID NO: 2; G, A at a residue corresponding to position 369 in SEQ ID NO: 2; Y, F at a residue corresponding to position 370 in SEQ ID NO: 2; L, Q at a residue corresponding to position 372 in SEQ ID NO: 2; S at a residue corresponding to position 373 in SEQ ID NO: 2; A, C at a residue corresponding to position 375 in SEQ ID NO: 2; T, S, C, G at a residue corresponding to position 376 in SEQ ID NO: 2; G, M at a residue corresponding to position 377 in SEQ ID NO: 2; C, T, V at a residue corresponding to position 378 in SEQ ID NO: 2; C, V, I, L, A at a residue corresponding to position 379 in SEQ ID NO: 2; F, C, H, S, M, T, V at a residue corresponding to position 380 in SEQ ID NO: 2; N, F, L, C, V at a residue corresponding to position 381 in SEQ ID NO: 2; L, N, C, T, P at a residue corresponding to position 382 in SEQ ID NO: 2; D, N, R, P, Y at a residue corresponding to position 383 in SEQ ID NO: 2; P, N, D, W at a residue corresponding to position 384 in SEQ ID NO: 2; G, D at a residue corresponding to position 385 in SEQ ID NO: 2; D, V, L, T at a residue corresponding to position 386 in SEQ ID NO: 2; D, R, N, H, E at a residue corresponding to position 387 in SEQ ID NO: 2; K, H at a residue corresponding to position 388 in SEQ ID NO: 2; A, P, L at a residue corresponding to position 389 in SEQ ID NO: 2; S, T at a residue corresponding to position 391 in SEQ ID NO: 2; V, C at a residue corresponding to position 392 in SEQ ID NO: 2; R, K, I, Q, L at a residue corresponding to position 394 in SEQ ID NO: 2; A at a residue corresponding to position 395 in SEQ ID NO: 2; F, I, M, V at a residue corresponding to position 396 in SEQ ID NO: 2; D, H, N at a residue corresponding to position 398 in SEQ ID NO: 2; M, V, T, C, I at a residue corresponding to position 399 in SEQ ID NO: 2; D, Q at a residue corresponding to position 400 in SEQ ID NO: 2; M, I, C, L, A at a residue corresponding to position 401 in SEQ ID NO: 2; R at a residue corresponding to position 402 in SEQ ID NO: 2; V at a residue corresponding to position 404 in SEQ ID NO: 2; D at a residue corresponding to position 405 in SEQ ID NO: 2; E at a residue corresponding to position 406 in SEQ ID NO: 2; P at a residue corresponding to position 407 in SEQ ID NO: 2; Q, E, G at a residue corresponding to position 409 in SEQ ID NO: 2; D, S, V, G, N at a residue corresponding to position 411 in SEQ ID NO: 2; I, V at a residue corresponding to position 413 in SEQ ID NO: 2; W, L, V, C, Q at a residue corresponding to position 414 in SEQ ID NO: 2; I at a residue corresponding to position 415 in SEQ ID NO: 2; R at a residue corresponding to position 416 in SEQ ID NO: 2; S at a residue corresponding to position 417 in SEQ ID NO: 2; D at a residue corresponding to position 418 in SEQ ID NO: 2; H, S, Q, M at a residue corresponding to position 419 in SEQ ID NO: 2; V at a residue corresponding to position 420 in SEQ ID NO: 2; T, F at a residue corresponding to position 421 in SEQ ID NO: 2; H, L at a residue corresponding to position 425 in SEQ ID NO: 2; A at a residue corresponding to position 430 in SEQ ID NO: 2; K at a residue corresponding to position 432 in SEQ ID NO: 2; R, D at a residue corresponding to position 433 in SEQ ID NO: 2; T, V at a residue corresponding to position 434 in SEQ ID NO: 2; I, M, L at a residue corresponding to position 435 in SEQ ID NO: 2; F, M, Y at a residue corresponding to position 441 in SEQ ID NO: 2; K, H, Y, R, C at a residue corresponding to position 442 in SEQ ID NO: 2; S at a residue corresponding to position 443 in SEQ ID NO: 2; M, I, L, V at a residue corresponding to position 446 in SEQ ID NO: 2; A, G at a residue corresponding to position 447 in SEQ ID NO: 2; I at a residue corresponding to position 449 in SEQ ID NO: 2; D, M at a residue corresponding to position 450 in SEQ ID NO: 2; E at a residue corresponding to position 451 in SEQ ID NO: 2; P at a residue corresponding to position 452 in SEQ ID NO: 2; G at a residue corresponding to position 453 in SEQ ID NO: 2; E, G, R at a residue corresponding to position 455 in SEQ ID NO: 2; D, E, Q, N at a residue corresponding to position 456 in SEQ ID NO: 2; N, F, Y, W at a residue corresponding to position 458 in SEQ ID NO: 2; Y at a residue corresponding to position 459 in SEQ ID NO: 2; Y, F, I, R, W at a residue corresponding to position 460 in SEQ ID NO: 2; I, L, Y at a residue corresponding to position 461 in SEQ ID NO: 2; I, T, V at a residue corresponding to position 462 in SEQ ID NO: 2; D at a residue corresponding to position 463 in SEQ ID NO: 2; D at a residue corresponding to position 464 in SEQ ID NO: 2; L at a residue corresponding to position 465 in SEQ ID NO: 2; D, K, A at a residue corresponding to position 466 in SEQ ID NO: 2; K at a residue corresponding to position 467 in SEQ ID NO: 2; M at a residue corresponding to position 468 in SEQ ID NO: 2; L at a residue corresponding to position 469 in SEQ ID NO: 2; V at a residue corresponding to position 470 in SEQ ID NO: 2; T, V at a residue corresponding to position 471 in SEQ ID NO: 2; S, N at a residue corresponding to position 473 in SEQ ID NO: 2; E at a residue corresponding to position 474 in SEQ ID NO: 2; N, Y, L, F, V, I at a residue corresponding to position 476 in SEQ ID NO: 2; T, L at a residue corresponding to position 478 in SEQ ID NO: 2; P, V at a residue corresponding to position 479 in SEQ ID NO: 2; L, I, S, P, A, V at a residue corresponding to position 480 in SEQ ID NO: 2; P, E at a residue corresponding to position 481 in SEQ ID NO: 2; E at a residue corresponding to position 483 in SEQ ID NO: 2; A, L, E, R at a residue corresponding to position 485 in SEQ ID NO: 2; C at a residue corresponding to position 490 in SEQ ID NO: 2; P at a residue corresponding to position 492 in SEQ ID NO: 2; F at a residue corresponding to position 493 in SEQ ID NO: 2; H at a residue corresponding to position 496 in SEQ ID NO: 2; A at a residue corresponding to position 497 in SEQ ID NO: 2; C at a residue corresponding to position 498 in SEQ ID NO: 2; P at a residue corresponding to position 502 in SEQ ID NO: 2; H at a residue corresponding to position 504 in SEQ ID NO: 2; Y, V, W at a residue corresponding to position 507 in SEQ ID NO: 2; V at a residue corresponding to position 508 in SEQ ID NO: 2; A at a residue corresponding to position 510 in SEQ ID NO: 2; V, L at a residue corresponding to position 511 in SEQ ID NO: 2; I at a residue corresponding to position 512 in SEQ ID NO: 2; V at a residue corresponding to position 513 in SEQ ID NO: 2; N at a residue corresponding to position 514 in SEQ ID NO: 2; D, S at a residue corresponding to position 515 in SEQ ID NO: 2; A at a residue corresponding to position 516 in SEQ ID NO: 2; I at a residue corresponding to position 518 in SEQ ID NO: 2; T at a residue corresponding to position 519 in SEQ ID NO: 2; P, W at a residue corresponding to position 521 in SEQ ID NO: 2; L, M at a residue corresponding to position 526 in SEQ ID NO: 2; L at a residue corresponding to position 527 in SEQ ID NO: 2; R, H at a residue corresponding to position 532 in SEQ ID NO: 2; L at a residue corresponding to position 535 in SEQ ID NO: 2; Q at a residue corresponding to position 540 in SEQ ID NO: 2; Y, W at a residue corresponding to position 542 in SEQ ID NO: 2; N at a residue corresponding to position 546 in SEQ ID NO: 2; Q at a residue corresponding to position 552 in SEQ ID NO: 2; E at a residue corresponding to position 554 in SEQ ID NO: 2; F at a residue corresponding to position 558 in SEQ ID NO: 2; D at a residue corresponding to position 560 in SEQ ID NO: 2; D at a residue corresponding to position 561 in SEQ ID NO: 2; F, L, W at a residue corresponding to position 563 in SEQ ID NO: 2; D, K, S, L, T, R at a residue corresponding to position 564 in SEQ ID NO: 2; M, I, V, P, T at a residue corresponding to position 565 in SEQ ID NO: 2; A, E, D, P at a residue corresponding to position 566 in SEQ ID NO: 2; K, Q, T, N, S, A at a residue corresponding to position 567 in SEQ ID NO: 2; G, N, D at a residue corresponding to position 568 in SEQ ID NO: 2; M, K at a residue corresponding to position 569 in SEQ ID NO: 2; V, L at a residue corresponding to position 570 in SEQ ID NO: 2; R, P at a residue corresponding to position 572 in SEQ ID NO: 2; T, S at a residue corresponding to position 573 in SEQ ID NO: 2; M, L at a residue corresponding to position 574 in SEQ ID NO: 2; A, Y, K, T at a residue corresponding to position 575 in SEQ ID NO: 2; Y, A, L at a residue corresponding to position 577 in SEQ ID NO: 2; Q, A at a residue corresponding to position 578 in SEQ ID NO: 2; D at a residue corresponding to position 583 in SEQ ID NO: 2; N at a residue corresponding to position 584 in SEQ ID NO: 2; H, L at a residue corresponding to position 585 in SEQ ID NO: 2; A at a residue corresponding to position 587 in SEQ ID NO: 2; Q at a residue corresponding to position 588 in SEQ ID NO: 2; Q, E at a residue corresponding to position 591 in SEQ ID NO: 2; A at a residue corresponding to position 593 in SEQ ID NO: 2; E at a residue corresponding to position 595 in SEQ ID NO: 2; K at a residue corresponding to position 596 in SEQ ID NO: 2; D at a residue corresponding to position 598 in SEQ ID NO: 2; S at a residue corresponding to position 599 in SEQ ID NO: 2; L at a residue corresponding to position 601 in SEQ ID NO: 2; or any combination thereof. It is understood that when listed herein, alterations listed for a certain position can be in the alternative or in the conjunctive depending on the context. For example, the V, F, FV alterations at a residue corresponding to position 565 in SEQ ID NO: 2, indicates that alterations at a residue corresponding to position 565 in SEQ ID NO: 2 can comprise V or F or FV, wherein FV is F and V.

In some embodiments, provided herein is microbial organism (e.g., host microbial organism) that has a recombinant nucleic acid encoding an engineered ACL an engineered ACL provided herein has an amino acid sequence that is a variant of SEQ ID NO: 2 that includes one or more alterations as described in TABLE 9 and/or one or more or a combination of alterations described in TABLE 8, wherein the portion, other than the one or more alterations described in TABLE 9 or the one or more or combination of alterations described in TABLE 8, of the engineered ACL has at least 65%, 70%, 75%, 80%, 85%, 90%, 95%, 98% or 99% sequence identity, or is identical, to an amino acid sequence referenced as SEQ ID NO: 2. Accordingly, in some embodiments, provided herein is microbial organism (e.g., host microbial organism) that has a recombinant nucleic acid encoding an engineered ACL that has an amino acid sequence that includes one or more alterations as described in TABLE 9 and/or one or more or a combination of alterations described in TABLE 8 and the portion, other than the alteration described in TABLE 9 or the one or more or combination of alterations described in TABLE 8, of the engineered ACL has at least 65% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and/or one or more or a combination of alterations described in TABLE 8 and the portion, other than the alteration described in TABLE 9 or the one or more or combination of alterations described in TABLE 8, of the engineered ACL has at least 70% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and/or one or more or a combination of alterations described in TABLE 8 and the portion, other than the alteration described in TABLE 9 or the one or more or combination of alterations described in TABLE 8, of the engineered ACL has at least 75% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and/or one or more or a combination of alterations described in TABLE 8 and the portion, other than the alteration described in TABLE 9 or the one or more or combination of alterations described in TABLE 8, of the engineered ACL has at least 80% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and/or one or more or a combination of alterations described in TABLE 8 and the portion, other than the alteration described in TABLE 9 or the one or more or combination of alterations described in TABLE 8, of the engineered ACL has at least 85% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and/or one or more or a combination of alterations described in TABLE 8 and the portion, other than the alteration described in TABLE 9 or the one or more or combination of alterations described in TABLE 8, of the engineered ACL has at least 90% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and/or one or more or a combination of alterations described in TABLE 8 and the portion, other than the alteration described in TABLE 9 or the one or more or combination of alterations described in TABLE 8, of the engineered ACL has at least 95% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and/or one or more or a combination of alterations described in TABLE 8 and the portion, other than the alteration described in TABLE 9 or the one or more or combination of alterations described in TABLE 8, of the engineered ACL has at least 98% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and/or one or more or a combination of alterations described in TABLE 8 and the portion, other than the alteration described in TABLE 9 or the one or more or combination of alterations described in TABLE 8, of the engineered ACL has at least 99% identical to SEQ ID NO: 2. In some embodiments, provided herein is microbial organism (e.g., host microbial organism) that has a recombinant nucleic acid encodes an engineered ACL that has an amino acid sequence that includes one or more alterations as described in TABLE 9 and/or one or more or a combination of alterations described in TABLE 8 and the portion, other than the alteration described in TABLE 9 or the one or more or combination of alterations described in TABLE 8, of the engineered ACL is identical to SEQ ID NO: 2.

In one embodiment, the cell comprises a pathway that produces HHA, HHA-CoA, 6-HH, HDO, an ester or amide of HDO, 6-ACA, 6ACA-CoA, 6ACA-SADH, CPO, and/or HMD. In one embodiment, the cell is capable of fermentation. In one embodiment, the cell further includes at least one substrate for the engineered ACL described herein present or produced in the cell. In some embodiments, the substrate is HHA. In some embodiments, the cell has higher activity for HHA over propionate, acetate, adipate, succinate, or 4-hydroxybutyrate (4-TB), or any combination thereof. In one embodiment, the cell further includes at least one substrate for the engineered ACL described herein present or produced in the cell. In some embodiments, the substrate is 6-ACA. In some embodiments, the cell has higher activity for 6-ACA over propionate, acetate, adipate, succinate, or 4-hydroxybutyrate (4-HB), or any combination thereof.

Also provided herein is a culture medium comprising a cell described herein.

In some embodiments, provided herein is microbial organism (e.g., host microbial organism) that has a recombinant nucleic acid encoding an engineered ALDH that is a variant of a reference polypeptide, wherein the reference polypeptide has an amino acid sequence of SEQ ID NO: 3, and the engineered ALDH has one or more alterations at a position described in TABLE 12 relative to SEQ ID NO: 3. In some embodiments, the microbial organism has a recombinant nucleic acid that encodes an engineered ALDH that includes one or more amino acid alterations at a residue corresponding to position 2, 3, 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, 36, 37, 38, 39, 40, 41, 42, 43, 44, 45, 47, 48, 49, 50, 51, 52, 53, 54, 55, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 104, 106, 107, 108, 109, 110, 111, 112, 113, 114, 115, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, 201, 202, 203, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 243, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 288, 289, 290, 291, 292, 293, 294, 295, 296, 297, 298, 299, 300, 301, 302, 303, 304, 305, 306, 307, 308, 310, 311, 312, 313, 314, 315, 316, 317, 318, 319, 320, 321, 322, 323, 324, 325, 326, 327, 328, 329, 330, 331, 332, 333, 334, 335, 337, 338, 339, 341, 342, 343, 344, 345, 346, 347, 348, 349, 350, 351, 352, 354, 355, 356, 357, 358, 359, 360, 361, 362, 363, 364, 366, 368, 369, 370, 371, 372, 373, 374, 375, 376, 377, 378, 379, 380, 381, 382, 383, 384, 385, 386, 387, 388, 389, 390, 391, 392, 393, 394, 396, 397, 398, 399, 400, 401, 402, 403, 405, 406, 407, 408, 410, 411, 412, 413, 414, 415, 416, 418, 420, 421, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, more than one of the foregoing, or a combination thereof, in SEQ ID NO: 3. In some embodiments, the microbial organism has a recombinant nucleic acid that encodes an engineered ALDH that includes one or more amino acid alterations at a residue corresponding to position 2, 3, 4, 5, 6, 7, 9, 11, 12, 15, 16, 17, 18, 20, 21, 22, 23, 24, 25, 26, 27, 28, 30, 31, 32, 33, 34, 35, 36, 37, 38, 39, 40, 41, 42, 43, 44, 45, 47, 48, 49, 51, 52, 54, 55, 58, 61, 62, 63, 64, 65, 66, 67, 70, 71, 73, 74, 75, 77, 78, 79, 80, 81, 82, 83, 84, 86, 88, 89, 90, 91, 92, 93, 94, 95, 96, 97, 98, 99, 100, 101, 102, 104, 107, 111, 117, 119, 120, 121, 122, 124, 125, 126, 128, 129, 130,133, 136, 144, 145, 146, 147, 148, 149, 150, 151, 152, 153, 154, 155, 156, 157, 158, 159, 160, 162, 164, 165, 166, 167, 168, 169, 170, 171, 172, 174, 175, 176, 177, 180, 181, 183, 184, 187, 189, 190, 194, 196, 197, 198, 199, 200, 201, 202, 203, 206, 207, 208, 212, 214, 215, 216, 217, 218, 219, 221, 222, 223, 224, 225, 227, 228, 229, 230, 231, 232, 233, 234, 237, 238, 240, 243, 245, 246, 248, 249, 250, 251, 252, 253, 254, 255, 256, 257, 258, 259, 260, 261, 262, 263, 264, 265, 266, 267, 270, 271, 272, 273, 274, 275, 276, 277, 278, 279, 280, 281, 282, 283, 284, 285, 286, 288, 290, 291, 293, 294, 295, 296, 299, 300, 301, 302, 303, 304, 305, 306, 307, 308, 310, 311, 312, 313, 315, 316, 317, 318, 319, 320, 321, 322, 323, 324, 325, 326, 327, 328, 329, 330, 331, 332, 333, 334, 335, 339, 341, 342, 343, 344, 345, 346, 347, 348, 349, 350, 351, 354, 355, 356, 358, 359, 360, 362, 363, 366, 370, 371, 372, 373, 374, 375, 376, 377, 378, 379, 380, 381, 382, 383, 384, 385, 386, 387, 388, 389, 390, 392, 393, 394, 396, 397, 400, 401, 402, 405, 406, 407, 410, 411, 414, 420, 421, 423, 425, 426, 427, 428, 431, 433, 435, 436, 437, 438, 439, 440, 441, 443, 444, 446, 447, 448, 449, 450, 452, 453, 454, 455, 457, 458, 459, more than one of the foregoing, or a combination thereof, in SEQ ID NO: 3. In some embodiments, the microbial organism has a recombinant nucleic acid that encodes an engineered ALDH that includes one or more amino acid alterations at a residue corresponding to position 42, 119, 149, 158, 174, 246, 259, 263, 299, 327, 348, 370, 392, 401, 427, or a combination thereof, in SEQ ID NO: 3.

In some embodiments, provided herein is microbial organism (e.g., host microbial organism) that has a recombinant nucleic acid encoding an engineered ALDH having one or more alterations described in TABLE 12. Accordingly, in some embodiments, the microbial organism has a recombinant nucleic acid that encodes an engineered ALDH having: P, Q, T at a residue corresponding to position 2 in SEQ ID NO: 3; E, K, M, R, S at a residue corresponding to position 3 in SEQ ID NO: 3; G, S at a residue corresponding to position 4 in SEQ ID NO: 3; I, T at a residue corresponding to position 5 in SEQ ID NO: 3; E, F, H, P, Q, R at a residue corresponding to position 6 in SEQ ID NO: 3; E, Q at a residue corresponding to position 7 in SEQ ID NO: 3; D at a residue corresponding to position 8 in SEQ ID NO: 3; I, V at a residue corresponding to position 9 in SEQ ID NO: 3; N at a residue corresponding to position 10 in SEQ ID NO: 3; A, D, E, Q, R at a residue corresponding to position 11 in SEQ ID NO: 3; A, I, L at a residue corresponding to position 12 in SEQ ID NO: 3; L at a residue corresponding to position 13 in SEQ ID NO: 3; D, E, K, Q at a residue corresponding to position 14 in SEQ ID NO: 3; K, R at a residue corresponding to position 15 in SEQ ID NO: 3; A at a residue corresponding to position 16 in SEQ ID NO: 3; H, K, Q at a residue corresponding to position 17 in SEQ ID NO: 3; E, K, Q, T at a residue corresponding to position 18 in SEQ ID NO: 3; F, H, M at a residue corresponding to position 20 in SEQ ID NO: 3; A, E, K, P, R at a residue corresponding to position 21 in SEQ ID NO: 3; A, K, Q at a residue corresponding to position 22 in SEQ ID NO: 3; L, W, Y at a residue corresponding to position 23 in SEQ ID NO: 3; E, K, R, S at a residue corresponding to position 24 in SEQ ID NO: 3; A, E, K, N, S at a residue corresponding to position 25 in SEQ ID NO: 3; F, L, M, T, W at a residue corresponding to position 26 in SEQ ID NO: 3; D, S, T at a residue corresponding to position 27 in SEQ ID NO: 3; W at a residue corresponding to position 28 in SEQ ID NO: 3; K, Q at a residue corresponding to position 29 in SEQ ID NO: 3; D, E, K, Q, R at a residue corresponding to position 30 in SEQ ID NO: 3; I, L, R at a residue corresponding to position 31 in SEQ ID NO: 3; A, E, N at a residue corresponding to position 32 in SEQ ID NO: 3; A, E, Q, R at a residue corresponding to position 33 in SEQ ID NO: 3; I, L, M, V at a residue corresponding to position 34 in SEQ ID NO: 3; C, I, L, M at a residue corresponding to position 35 in SEQ ID NO: 3; E, H, K at a residue corresponding to position 36 in SEQ ID NO: 3; A, K, R at a residue corresponding to position 37 in SEQ ID NO: 3; F, I, L, M, W at a residue corresponding to position 38 in SEQ ID NO: 3; A, R at a residue corresponding to position 39 in SEQ ID NO: 3; D, E, Q, R, W at a residue corresponding to position 40 in SEQ ID NO: 3; L, V at a residue corresponding to position 41 in SEQ ID NO: 3; C, L, M, V at a residue corresponding to position 42 in SEQ ID NO: 3; E, L, R at a residue corresponding to position 43 in SEQ ID NO: 3; E, K, Q, R at a residue corresponding to position 44 in SEQ ID NO: 3; H, Q, R at a residue corresponding to position 45 in SEQ ID NO: 3; D at a residue corresponding to position 47 in SEQ ID NO: 3; D, E, M, R at a residue corresponding to position 48 in SEQ ID NO: 3; F, I, M, W, Y at a residue corresponding to position 49 in SEQ ID NO: 3; G at a residue corresponding to position 50 in SEQ ID NO: 3; A, E, K, N, Q, R at a residue corresponding to position 51 in SEQ ID NO: 3; L at a residue corresponding to position 52 in SEQ ID NO: 3; E, I, S at a residue corresponding to position 53 in SEQ ID NO: 3; I, L, M, T at a residue corresponding to position 54 in SEQ ID NO: 3; E, Q, R at a residue corresponding to position 55 in SEQ ID NO: 3; E, G, Q, R at a residue corresponding to position 58 in SEQ ID NO: 3; F, I, L, Y at a residue corresponding to position 59 in SEQ ID NO: 3; A at a residue corresponding to position 60 in SEQ ID NO: 3; D, E, K, R at a residue corresponding to position 61 in SEQ ID NO: 3; A, F, I, M, V, W at a residue corresponding to position 62 in SEQ ID NO: 3; D, E, Q at a residue corresponding to position 63 in SEQ ID NO: 3; E, H at a residue corresponding to position 64 in SEQ ID NO: 3; E, R at a residue corresponding to position 65 in SEQ ID NO: 3; L, V at a residue corresponding to position 66 in SEQ ID NO: 3; E at a residue corresponding to position 67 in SEQ ID NO: 3; D, E at a residue corresponding to position 68 in SEQ ID NO: 3; H, I, L, M, N at a residue corresponding to position 69 in SEQ ID NO: 3; I, L, M, R at a residue corresponding to position 70 in SEQ ID NO: 3; D, E, F, H, Y at a residue corresponding to position 71 in SEQ ID NO: 3; A, R at a residue corresponding to position 72 in SEQ ID NO: 3; A, T at a residue corresponding to position 73 in SEQ ID NO: 3; D, E, R at a residue corresponding to position 74 in SEQ ID NO: 3; G, P, Y at a residue corresponding to position 75 in SEQ ID NO: 3; F, I, L, M, Y at a residue corresponding to position 76 in SEQ ID NO: 3; F, Y at a residue corresponding to position 77 in SEQ ID NO: 3; E, F, H, N, R, Y at a residue corresponding to position 78 in SEQ ID NO: 3; D, S, W, Y at a residue corresponding to position 79 in SEQ ID NO: 3; F, I, L, V at a residue corresponding to position 80 in SEQ ID NO: 3; A, E, R at a residue corresponding to position 81 in SEQ ID NO: 3; E, G, P at a residue corresponding to position 82 in SEQ ID NO: 3; E, Q at a residue corresponding to position 83 in SEQ ID NO: 3; Q, R at a residue corresponding to position 84 in SEQ ID NO: 3; A, T at a residue corresponding to position 85 in SEQ ID NO: 3; E, K, V at a residue corresponding to position 86 in SEQ ID NO: 3; T at a residue corresponding to position 87 in SEQ ID NO: 3; L, M, V at a residue corresponding to position 88 in SEQ ID NO: 3; L, P, V at a residue corresponding to position 89 in SEQ ID NO: 3; D, E, H, P, R, S at a residue corresponding to position 90 in SEQ ID NO: 3; D, E, Q at a residue corresponding to position 91 in SEQ ID NO: 3; D, M at a residue corresponding to position 92 in SEQ ID NO: 3; D, E at a residue corresponding to position 93 in SEQ ID NO: 3; A, D, G, N at a residue corresponding to position 94 in SEQ ID NO: 3; K, M, N, R at a residue corresponding to position 95 in SEQ ID NO: 3; H, K, N, R at a residue corresponding to position 96 in SEQ ID NO: 3; I, M, V at a residue corresponding to position 97 in SEQ ID NO: 3; I, M, T, Y at a residue corresponding to position 98 in SEQ ID NO: 3; K, Q, R, T at a residue corresponding to position 99 in SEQ ID NO: 3; Y, L, M, I at a residue corresponding to position 100 in SEQ ID NO: 3; K, Y, R, H at a residue corresponding to position 101 in SEQ ID NO: 3; E, Q, R, V at a residue corresponding to position 102 in SEQ ID NO: 3; V, Y, F, L, M at a residue corresponding to position 104 in SEQ ID NO: 3; I at a residue corresponding to position 106 in SEQ ID NO: 3; C, V at a residue corresponding to position 107 in SEQ ID NO: 3; A, C at a residue corresponding to position 108 in SEQ ID NO: 3; I, A, M at a residue corresponding to position 109 in SEQ ID NO: 3; M, L, I at a residue corresponding to position 110 in SEQ ID NO: 3; T, V, Y, I, S at a residue corresponding to position 111 in SEQ ID NO: 3; E at a residue corresponding to position 112 in SEQ ID NO: 3; F, S, W, Y at a residue corresponding to position 113 in SEQ ID NO: 3; N at a residue corresponding to position 114 in SEQ ID NO: 3; W, R, H, Y at a residue corresponding to position 115 in SEQ ID NO: 3; N, L, T, I at a residue corresponding to position 117 in SEQ ID NO: 3; N, A, L, M, S, F, V at a residue corresponding to position 118 in SEQ ID NO: 3; Q, I, L, M, T, S at a residue corresponding to position 119 in SEQ ID NO: 3; T, A, P, M at a residue corresponding to position 120 in SEQ ID NO: 3; V, A, L, C, I at a residue corresponding to position 121 in SEQ ID NO: 3; C, F, R, Y, H, N, S, G, V, W at a residue corresponding to position 122 in SEQ ID NO: 3; K, P at a residue corresponding to position 123 in SEQ ID NO: 3; V, L, M, I at a residue corresponding to position 124 in SEQ ID NO: 3; I, L, A, V at a residue corresponding to position 125 in SEQ ID NO: 3; A, P at a residue corresponding to position 126 in SEQ ID NO: 3; M at a residue corresponding to position 127 in SEQ ID NO: 3; I at a residue corresponding to position 128 in SEQ ID NO: 3; A, M at a residue corresponding to position 129 in SEQ ID NO: 3; T, S, A at a residue corresponding to position 130 in SEQ ID NO: 3; R at a residue corresponding to position 131 in SEQ ID NO: 3; C at a residue corresponding to position 132 in SEQ ID NO: 3; T, C at a residue corresponding to position 133 in SEQ ID NO: 3; I, C, V at a residue corresponding to position 134 in SEQ ID NO: 3; V, M at a residue corresponding to position 135 in SEQ ID NO: 3; L, M, F, I at a residue corresponding to position 136 in SEQ ID NO: 3; R, K at a residue corresponding to position 137 in SEQ ID NO: 3; G at a residue corresponding to position 138 in SEQ ID NO: 3; S, G at a residue corresponding to position 139 in SEQ ID NO: 3; E at a residue corresponding to position 140 in SEQ ID NO: 3; Q, K, Y, E at a residue corresponding to position 141 in SEQ ID NO: 3; T, S at a residue corresponding to position 142 in SEQ ID NO: 3; P at a residue corresponding to position 143 in SEQ ID NO: 3; L, N, R at a residue corresponding to position 144 in SEQ ID NO: 3; T, C, V at a residue corresponding to position 145 in SEQ ID NO: 3; T, C, A, N at a residue corresponding to position 146 in SEQ ID NO: 3; I, M, A at a residue corresponding to position 147 in SEQ ID NO: 3; Y, L, A, E, H, R at a residue corresponding to position 148 in SEQ ID NO: 3; V, I, L, M, F at a residue corresponding to position 149 in SEQ ID NO: 3; I, M, G, A at a residue corresponding to position 150 in SEQ ID NO: 3; D, K, R, Q at a residue corresponding to position 151 in SEQ ID NO: 3; L, M, I at a residue corresponding to position 152 in SEQ ID NO: 3; F, V, C, M at a residue corresponding to position 153 in SEQ ID NO: 3; H, A, Q, K, R at a residue corresponding to position 154 in SEQ ID NO: 3; R, K, D, Q at a residue corresponding to position 155 in SEQ ID NO: 3; I at a residue corresponding to position 156 in SEQ ID NO: 3; A, I, F at a residue corresponding to position 157 in SEQ ID NO: 3; E, V, K at a residue corresponding to position 158 in SEQ ID NO: 3; K, Q, D, E at a residue corresponding to position 159 in SEQ ID NO: 3; V, C, A at a residue corresponding to position 160 in SEQ ID NO: 3; I, A, F, Y, L, V at a residue corresponding to position 162 in SEQ ID NO: 3; D, A, P at a residue corresponding to position 164 in SEQ ID NO: 3; H, G, D at a residue corresponding to position 165 in SEQ ID NO: 3; C, A, I, V at a residue corresponding to position 166 in SEQ ID NO: 3; V, F, L at a residue corresponding to position 167 in SEQ ID NO: 3; T, N at a residue corresponding to position 168 in SEQ ID NO: 3; L, W, C, M, I at a residue corresponding to position 169 in SEQ ID NO: 3; L, V at a residue corresponding to position 170 in SEQ ID NO: 3; E, P, N, D at a residue corresponding to position 171 in SEQ ID NO: 3; D, G, H at a residue corresponding to position 172 in SEQ ID NO: 3; G, D at a residue corresponding to position 173 in SEQ ID NO: 3; G, D, T at a residue corresponding to position 174 in SEQ ID NO: 3; I, H, R at a residue corresponding to position 175 in SEQ ID NO: 3; Q, D at a residue corresponding to position 176 in SEQ ID NO: 3; V, T, A, I at a residue corresponding to position 177 in SEQ ID NO: 3; V, A, T, G at a residue corresponding to position 178 in SEQ ID NO: 3; D, N, E at a residue corresponding to position 179 in SEQ ID NO: 3; H, Q, A, E at a residue corresponding to position 180 in SEQ ID NO: 3; I, F at a residue corresponding to position 181 in SEQ ID NO: 3; L, H, I, V at a residue corresponding to position 182 in SEQ ID NO: 3; Q, K, D, A, H, E at a residue corresponding to position 183 in SEQ ID NO: 3; H, P, S, G at a residue corresponding to position 184 in SEQ ID NO: 3; C, I at a residue corresponding to position 185 in SEQ ID NO: 3; R at a residue corresponding to position 186 in SEQ ID NO: 3; H, K, F, I, V, M at a residue corresponding to position 187 in SEQ ID NO: 3; I, L, V at a residue corresponding to position 188 in SEQ ID NO: 3; S, F, T, M, V, L at a residue corresponding to position 189 in SEQ ID NO: 3; P, A, F at a residue corresponding to position 190 in SEQ ID NO: 3; R at a residue corresponding to position 191 in SEQ ID NO: 3; S at a residue corresponding to position 193 in SEQ ID NO: 3; T, R, K, A at a residue corresponding to position 194 in SEQ ID NO: 3; A at a residue corresponding to position 195 in SEQ ID NO: 3; M, I, V at a residue corresponding to position 196 in SEQ ID NO: 3; I, M, G, A at a residue corresponding to position 197 in SEQ ID NO: 3; Q, E, R at a residue corresponding to position 198 in SEQ ID NO: 3; S, V at a residue corresponding to position 199 in SEQ ID NO: 3; C, I, V at a residue corresponding to position 200 in SEQ ID NO: 3; M, Q, A at a residue corresponding to position 201 in SEQ ID NO: 3; N, K, A, R, T, E, Q at a residue corresponding to position 202 in SEQ ID NO: 3; N, H, A at a residue corresponding to position 203 in SEQ ID NO: 3; R, T at a residue corresponding to position 205 in SEQ ID NO: 3; V, R, K at a residue corresponding to position 206 in SEQ ID NO: 3; V, S, C, T at a residue corresponding to position 207 in SEQ ID NO: 3; V, T, Y, M, L at a residue corresponding to position 208 in SEQ ID NO: 3; L, E, M at a residue corresponding to position 209 in SEQ ID NO: 3; H, L at a residue corresponding to position 210 in SEQ ID NO: 3; L at a residue corresponding to position 211 in SEQ ID NO: 3; A, P, G at a residue corresponding to position 212 in SEQ ID NO: 3; K at a residue corresponding to position 213 in SEQ ID NO: 3; S, K at a residue corresponding to position 214 in SEQ ID NO: 3; S, N, A, C, D, P at a residue corresponding to position 215 in SEQ ID NO: 3; H, A, P at a residue corresponding to position 216 in SEQ ID NO: 3; F, A, I, C, M at a residue corresponding to position 217 in SEQ ID NO: 3; I, V, F, Y at a residue corresponding to position 218 in SEQ ID NO: 3; M, F, L, I at a residue corresponding to position 219 in SEQ ID NO: 3; C, F at a residue corresponding to position 220 in SEQ ID NO: 3; D, P at a residue corresponding to position 221 in SEQ ID NO: 3; T, S at a residue corresponding to position 222 in SEQ ID NO: 3; C at a residue corresponding to position 223 in SEQ ID NO: 3; N at a residue corresponding to position 224 in SEQ ID NO: 3; V at a residue corresponding to position 225 in SEQ ID NO: 3; E at a residue corresponding to position 226 in SEQ ID NO: 3; K, R, M, E at a residue corresponding to position 227 in SEQ ID NO: 3; A at a residue corresponding to position 228 in SEQ ID NO: 3; W, V at a residue corresponding to position 229 in SEQ ID NO: 3; R, D, P, Q, K, E at a residue corresponding to position 230 in SEQ ID NO: 3; Q, R, I, K at a residue corresponding to position 231 in SEQ ID NO: 3; I, F, T, C, Q, M, N at a residue corresponding to position 232 in SEQ ID NO: 3; V, L, M at a residue corresponding to position 233 in SEQ ID NO: 3; W, Q, F, A, M at a residue corresponding to position 234 in SEQ ID NO: 3; A, S at a residue corresponding to position 235 in SEQ ID NO: 3; K, C, W, A, I at a residue corresponding to position 236 in SEQ ID NO: 3; F, Y, C, M at a residue corresponding to position 237 in SEQ ID NO: 3; R, M, Q, Y, W at a residue corresponding to position 238 in SEQ ID NO: 3; R, H, Q at a residue corresponding to position 239 in SEQ ID NO: 3; Q, A, T, C, S at a residue corresponding to position 240 in SEQ ID NO: 3; N at a residue corresponding to position 241 in SEQ ID NO: 3; V, M, T, C at a residue corresponding to position 243 in SEQ ID NO: 3; M, I, N, S, V, A, C at a residue corresponding to position 245 in SEQ ID NO: 3; S, A at a residue corresponding to position 246 in SEQ ID NO: 3; G, P, I, A at a residue corresponding to position 247 in SEQ ID NO: 3; D, T, E, K, S, N at a residue corresponding to position 248 in SEQ ID NO: 3; Y, R at a residue corresponding to position 249 in SEQ ID NO: 3; I, L, M at a residue corresponding to position 250 in SEQ ID NO: 3; Y, F, L, V at a residue corresponding to position 251 in SEQ ID NO: 3; V, I at a residue corresponding to position 252 in SEQ ID NO: 3; D, Q, H, V, E at a residue corresponding to position 253 in SEQ ID NO: 3; Q, K, R, D at a residue corresponding to position 254 in SEQ ID NO: 3; E at a residue corresponding to position 255 in SEQ ID NO: 3; V, M, I, R at a residue corresponding to position 256 in SEQ ID NO: 3; A, F, H, Y at a residue corresponding to position 257 in SEQ ID NO: 3; D at a residue corresponding to position 258 in SEQ ID NO: 3; A, R, K, D, Q at a residue corresponding to position 259 in SEQ ID NO: 3; I, V, F, L at a residue corresponding to position 260 in SEQ ID NO: 3; L, M, V at a residue corresponding to position 261 in SEQ ID NO: 3; Q, D, E, K at a residue corresponding to position 262 in SEQ ID NO: 3; E, K, R at a residue corresponding to position 263 in SEQ ID NO: 3; M, V, F at a residue corresponding to position 264 in SEQ ID NO: 3; Q, R, A, E at a residue corresponding to position 265 in SEQ ID NO: 3; A, E at a residue corresponding to position 266 in SEQ ID NO: 3; Y, R, N, A, Q at a residue corresponding to position 267 in SEQ ID NO: 3; A at a residue corresponding to position 268 in SEQ ID NO: 3; S, G, C at a residue corresponding to position 269 in SEQ ID NO: 3; L, F, I, M, W at a residue corresponding to position 270 in SEQ ID NO: 3; F, K, V, W, R, H at a residue corresponding to position 271 in SEQ ID NO: 3; V, M, L at a residue corresponding to position 272 in SEQ ID NO: 3; C at a residue corresponding to position 273 in SEQ ID NO: 3; E, D at a residue corresponding to position 274 in SEQ ID NO: 3; P, G, A, E at a residue corresponding to position 275 in SEQ ID NO: 3; Q, M, R, D at a residue corresponding to position 276 in SEQ ID NO: 3; Q, E at a residue corresponding to position 277 in SEQ ID NO: 3; P, E at a residue corresponding to position 278 in SEQ ID NO: 3; E, D, S at a residue corresponding to position 279 in SEQ ID NO: 3; Q, R, T at a residue corresponding to position 280 in SEQ ID NO: 3; V, M, I at a residue corresponding to position 281 in SEQ ID NO: 3; Q, K, M, G, E at a residue corresponding to position 282 in SEQ ID NO: 3; E, H, N at a residue corresponding to position 283 in SEQ ID NO: 3; V, P at a residue corresponding to position 284 in SEQ ID NO: 3; L, V, I, M at a residue corresponding to position 285 in SEQ ID NO: 3; L, I, W, Y, V at a residue corresponding to position 286 in SEQ ID NO: 3; R, D, E at a residue corresponding to position 288 in SEQ ID NO: 3; M at a residue corresponding to position 289 in SEQ ID NO: 3; H at a residue corresponding to position 290 in SEQ ID NO: 3; K, L, E, T, H, A, N at a residue corresponding to position 291 in SEQ ID NO: 3; I, M at a residue corresponding to position 292 in SEQ ID NO: 3; A, V, I, N at a residue corresponding to position 293 in SEQ ID NO: 3; S, N, P, E, R at a residue corresponding to position 294 in SEQ ID NO: 3; R, K, A, Q, D at a residue corresponding to position 295 in SEQ ID NO: 3; W, R, V, F, H, L at a residue corresponding to position 296 in SEQ ID NO: 3; A, T, Q, V, L, H, I at a residue corresponding to position 297 in SEQ ID NO: 3; A at a residue corresponding to position 298 in SEQ ID NO: 3; K, Q, R at a residue corresponding to position 299 in SEQ ID NO: 3; A, R at a residue corresponding to position 300 in SEQ ID NO: 3; I, A at a residue corresponding to position 301 in SEQ ID NO: 3; A, R, D, Q, E at a residue corresponding to position 302 in SEQ ID NO: 3; Q, W, R, E at a residue corresponding to position 303 in SEQ ID NO: 3; L, G, Y at a residue corresponding to position 304 in SEQ ID NO: 3; I, V at a residue corresponding to position 305 in SEQ ID NO: 3; H, K, E, R at a residue corresponding to position 306 in SEQ ID NO: 3; I, V, M at a residue corresponding to position 307 in SEQ ID NO: 3; A, T at a residue corresponding to position 308 in SEQ ID NO: 3; G, V, L, F at a residue corresponding to position 310 in SEQ ID NO: 3; R, T, K, S, D, E at a residue corresponding to position 311 in SEQ ID NO: 3; A, D, T, K at a residue corresponding to position 312 in SEQ ID NO: 3; L, V at a residue corresponding to position 313 in SEQ ID NO: 3; A at a residue corresponding to position 314 in SEQ ID NO: 3; D at a residue corresponding to position 315 in SEQ ID NO: 3; E, G, N at a residue corresponding to position 316 in SEQ ID NO: 3; G, T at a residue corresponding to position 317 in SEQ ID NO: 3; R, T, Y at a residue corresponding to position 318 in SEQ ID NO: 3; F, L, M, I, Y at a residue corresponding to position 319 in SEQ ID NO: 3; Y, L at a residue corresponding to position 320 in SEQ ID NO: 3; L, P, C, I at a residue corresponding to position 321 in SEQ ID NO: 3; A, T, G, V at a residue corresponding to position 322 in SEQ ID NO: 3; E, L, I, G, P, V, K at a residue corresponding to position 323 in SEQ ID NO: 3; I, V, L at a residue corresponding to position 324 in SEQ ID NO: 3; K, E, D, T at a residue corresponding to position 325 in SEQ ID NO: 3; D at a residue corresponding to position 326 in SEQ ID NO: 3; W, F, V at a residue corresponding to position 327 in SEQ ID NO: 3; D, T, S at a residue corresponding to position 328 in SEQ ID NO: 3; E, R, P, D, N at a residue corresponding to position 329 in SEQ ID NO: 3; E at a residue corresponding to position 330 in SEQ ID NO: 3; S, D, H, M, Y at a residue corresponding to position 331 in SEQ ID NO: 3; R, T at a residue corresponding to position 332 in SEQ ID NO: 3; M, V, F, I at a residue corresponding to position 333 in SEQ ID NO: 3; V, S, M, C at a residue corresponding to position 334 in SEQ ID NO: 3; R, Q, H at a residue corresponding to position 335 in SEQ ID NO: 3; Q, E at a residue corresponding to position 337 in SEQ ID NO: 3; F, V, I, M at a residue corresponding to position 338 in SEQ ID NO: 3; C, M at a residue corresponding to position 339 in SEQ ID NO: 3; I at a residue corresponding to position 341 in SEQ ID NO: 3; M, V, I at a residue corresponding to position 342 in SEQ ID NO: 3; C, T, V, S, P at a residue corresponding to position 343 in SEQ ID NO: 3; I, V, L, M at a residue corresponding to position 344 in SEQ ID NO: 3; Y, V, M, L, I at a residue corresponding to position 345 in SEQ ID NO: 3; R, K, P at a residue corresponding to position 346 in SEQ ID NO: 3; F, V at a residue corresponding to position 347 in SEQ ID NO: 3; E, K, S at a residue corresponding to position 348 in SEQ ID NO: 3; S, G, D, N, T at a residue corresponding to position 349 in SEQ ID NO: 3; V, E, I, L, F at a residue corresponding to position 350 in SEQ ID NO: 3; D at a residue corresponding to position 351 in SEQ ID NO: 3; Q, H, E at a residue corresponding to position 352 in SEQ ID NO: 3; L, V, C at a residue corresponding to position 354 in SEQ ID NO: 3; R, A, D, Q, S, E at a residue corresponding to position 355 in SEQ ID NO: 3; M, L, V at a residue corresponding to position 356 in SEQ ID NO: 3; C at a residue corresponding to position 357 in SEQ ID NO: 3; N, R at a residue corresponding to position 358 in SEQ ID NO: 3; A, D, S, N, E at a residue corresponding to position 359 in SEQ ID NO: 3; S, T at a residue corresponding to position 360 in SEQ ID NO: 3; D, V at a residue corresponding to position 361 in SEQ ID NO: 3; L, T, N, S at a residue corresponding to position 362 in SEQ ID NO: 3; Y, E at a residue corresponding to position 363 in SEQ ID NO: 3; Q, Y, F, H at a residue corresponding to position 364 in SEQ ID NO: 3; Y, K, F, A, L at a residue corresponding to position 366 in SEQ ID NO: 3; L, A at a residue corresponding to position 368 in SEQ ID NO: 3; T, A at a residue corresponding to position 369 in SEQ ID NO: 3; C, A at a residue corresponding to position 370 in SEQ ID NO: 3; M, I, A, W, S, Y at a residue corresponding to position 371 in SEQ ID NO: 3; V, M at a residue corresponding to position 372 in SEQ ID NO: 3; W, Y, F at a residue corresponding to position 373 in SEQ ID NO: 3; T at a residue corresponding to position 374 in SEQ ID NO: 3; H, Q, K, T, R at a residue corresponding to position 375 in SEQ ID NO: 3; N, S, D at a residue corresponding to position 376 in SEQ ID NO: 3; D, I, V, E at a residue corresponding to position 377 in SEQ ID NO: 3; K, D, A at a residue corresponding to position 378 in SEQ ID NO: 3; N, K, V, R at a residue corresponding to position 379 in SEQ ID NO: 3; A, M, V at a residue corresponding to position 380 in SEQ ID NO: 3; M, W, K, R, T, H at a residue corresponding to position 381 in SEQ ID NO: 3; K, R, E, Q at a residue corresponding to position 382 in SEQ ID NO: 3; M, L, Y, F, I at a residue corresponding to position 383 in SEQ ID NO: 3; S, G at a residue corresponding to position 384 in SEQ ID NO: 3; Q, L, R, K at a residue corresponding to position 385 in SEQ ID NO: 3; E, Q, K, R at a residue corresponding to position 386 in SEQ ID NO: 3; M, V, L at a residue corresponding to position 387 in SEQ ID NO: 3; R, H, D, Q, E at a residue corresponding to position 388 in SEQ ID NO: 3; A, C, T at a residue corresponding to position 389 in SEQ ID NO: 3; T, N, C, G at a residue corresponding to position 390 in SEQ ID NO: 3; M, T, I at a residue corresponding to position 391 in SEQ ID NO: 3; V, I, M at a residue corresponding to position 392 in SEQ ID NO: 3; M, I, W, L, G at a residue corresponding to position 393 in SEQ ID NO: 3; I, K at a residue corresponding to position 394 in SEQ ID NO: 3; R, E, S, D at a residue corresponding to position 396 in SEQ ID NO: 3; I, W, A, P, F, Y at a residue corresponding to position 397 in SEQ ID NO: 3; S, C, P, T at a residue corresponding to position 398 in SEQ ID NO: 3; A, S at a residue corresponding to position 399 in SEQ ID NO: 3; I, S, F, V, H at a residue corresponding to position 400 in SEQ ID NO: 3; G, H, D, S, A at a residue corresponding to position 401 in SEQ ID NO: 3; N, A, F, M at a residue corresponding to position 402 in SEQ ID NO: 3; S at a residue corresponding to position 403 in SEQ ID NO: 3; I, F, V, G at a residue corresponding to position 405 in SEQ ID NO: 3; Y, K, F, L, W at a residue corresponding to position 406 in SEQ ID NO: 3; H, Q, Y, E at a residue corresponding to position 407 in SEQ ID NO: 3; T, S, H at a residue corresponding to position 408 in SEQ ID NO: 3; F, I, M at a residue corresponding to position 410 in SEQ ID NO: 3; T, G, P, A at a residue corresponding to position 411 in SEQ ID NO: 3; F, R at a residue corresponding to position 412 in SEQ ID NO: 3; A at a residue corresponding to position 413 in SEQ ID NO: 3; M, F, V at a residue corresponding to position 414 in SEQ ID NO: 3; P, S at a residue corresponding to position 415 in SEQ ID NO: 3; M, F, I at a residue corresponding to position 416 in SEQ ID NO: 3; F, I, T at a residue corresponding to position 418 in SEQ ID NO: 3; S, R at a residue corresponding to position 420 in SEQ ID NO: 3; Y, F at a residue corresponding to position 421 in SEQ ID NO: 3; K, R, G at a residue corresponding to position 423 in SEQ ID NO: 3; S, R at a residue corresponding to position 424 in SEQ ID NO: 3; I, G, W, A at a residue corresponding to position 425 in SEQ ID NO: 3; V, T, L at a residue corresponding to position 426 in SEQ ID NO: 3; G, T, A at a residue corresponding to position 427 in SEQ ID NO: 3; D, H at a residue corresponding to position 428 in SEQ ID NO: 3; E at a residue corresponding to position 429 in SEQ ID NO: 3; I, Y, L, V at a residue corresponding to position 430 in SEQ ID NO: 3; G, S, N at a residue corresponding to position 431 in SEQ ID NO: 3; A, P, F, W at a residue corresponding to position 432 in SEQ ID NO: 3; T, R, K, H, M, E at a residue corresponding to position 433 in SEQ ID NO: 3; R, Q, D, N at a residue corresponding to position 434 in SEQ ID NO: 3; V, F, W, Y, M at a residue corresponding to position 435 in SEQ ID NO: 3; T, L, F, I, V at a residue corresponding to position 436 in SEQ ID NO: 3; H, Q, Y at a residue corresponding to position 437 in SEQ ID NO: 3; V, T, W, C, Y, I at a residue corresponding to position 438 in SEQ ID NO: 3; Q, S, R, K at a residue corresponding to position 439 in SEQ ID NO: 3; K, W at a residue corresponding to position 440 in SEQ ID NO: 3; L, I, M at a residue corresponding to position 441 in SEQ ID NO: 3; G, A at a residue corresponding to position 442 in SEQ ID NO: 3; H, W, R, F at a residue corresponding to position 443 in SEQ ID NO: 3; E, P, K at a residue corresponding to position 444 in SEQ ID NO: 3; I, Y, F, M, L at a residue corresponding to position 445 in SEQ ID NO: 3; E, K at a residue corresponding to position 446 in SEQ ID NO: 3; E, N at a residue corresponding to position 447 in SEQ ID NO: 3; W, K, P at a residue corresponding to position 448 in SEQ ID NO: 3; E, R, F, H at a residue corresponding to position 449 in SEQ ID NO: 3; M, C, F, V, L at a residue corresponding to position 450 in SEQ ID NO: 3; W at a residue corresponding to position 451 in SEQ ID NO: 3; S, D, K at a residue corresponding to position 452 in SEQ ID NO: 3; E, P, H at a residue corresponding to position 453 in SEQ ID NO: 3; A, D at a residue corresponding to position 454 in SEQ ID NO: 3; K, Q, D at a residue corresponding to position 455 in SEQ ID NO: 3; F, L, V, W, M at a residue corresponding to position 456 in SEQ ID NO: 3; M, Y, F at a residue corresponding to position 457 in SEQ ID NO: 3; H, G at a residue corresponding to position 458 in SEQ ID NO: 3; D at a residue corresponding to position 459 in SEQ ID NO: 3; more than one of the foregoing; or any combination thereof.

In some embodiments, provided herein is microbial organism (e.g., host microbial organism) that has a recombinant nucleic acid encoding an engineered ALDH having: Q, T, P at a residue corresponding to position 2 in SEQ ID NO: 3; S, K at a residue corresponding to position 3 in SEQ ID NO: 3; G, S at a residue corresponding to position 4 in SEQ ID NO: 3; I, T at a residue corresponding to position 5 in SEQ ID NO: 3; F at a residue corresponding to position 6 in SEQ ID NO: 3; E, Q at a residue corresponding to position 7 in SEQ ID NO: 3; V at a residue corresponding to position 9 in SEQ ID NO: 3; E, D, Q, R at a residue corresponding to position 11 in SEQ ID NO: 3; L, A at a residue corresponding to position 12 in SEQ ID NO: 3; R, K at a residue corresponding to position 15 in SEQ ID NO: 3; A at a residue corresponding to position 16 in SEQ ID NO: 3; K at a residue corresponding to position 17 in SEQ ID NO: 3; K, E, Q at a residue corresponding to position 18 in SEQ ID NO: 3; F, H at a residue corresponding to position 20 in SEQ ID NO: 3; A, R at a residue corresponding to position 21 in SEQ ID NO: 3; K, Q, A at a residue corresponding to position 22 in SEQ ID NO: 3; Y at a residue corresponding to position 23 in SEQ ID NO: 3; E, K at a residue corresponding to position 24 in SEQ ID NO: 3; A, E at a residue corresponding to position 25 in SEQ ID NO: 3; L, M, W at a residue corresponding to position 26 in SEQ ID NO: 3; D, T at a residue corresponding to position 27 in SEQ ID NO: 3; W at a residue corresponding to position 28 in SEQ ID NO: 3; D, K, Q at a residue corresponding to position 30 in SEQ ID NO: 3; I, L at a residue corresponding to position 31 in SEQ ID NO: 3; N, E at a residue corresponding to position 32 in SEQ ID NO: 3; E, Q, A, R at a residue corresponding to position 33 in SEQ ID NO: 3; V at a residue corresponding to position 34 in SEQ ID NO: 3; C, M at a residue corresponding to position 35 in SEQ ID NO: 3; K at a residue corresponding to position 36 in SEQ ID NO: 3; K, A, R at a residue corresponding to position 37 in SEQ ID NO: 3; L, I at a residue corresponding to position 38 in SEQ ID NO: 3; A at a residue corresponding to position 39 in SEQ ID NO: 3; W at a residue corresponding to position 40 in SEQ ID NO: 3; L, V at a residue corresponding to position 41 in SEQ ID NO: 3; L, V, M, C at a residue corresponding to position 42 in SEQ ID NO: 3; E, L at a residue corresponding to position 43 in SEQ ID NO: 3; K, Q at a residue corresponding to position 44 in SEQ ID NO: 3; H, R at a residue corresponding to position 45 in SEQ ID NO: 3; D at a residue corresponding to position 47 in SEQ ID NO: 3; E, M, D at a residue corresponding to position 48 in SEQ ID NO: 3; I at a residue corresponding to position 49 in SEQ ID NO: 3; N, E, Q at a residue corresponding to position 51 in SEQ ID NO: 3; L at a residue corresponding to position 52 in SEQ ID NO: 3; L at a residue corresponding to position 54 in SEQ ID NO: 3; E, R at a residue corresponding to position 55 in SEQ ID NO: 3; R at a residue corresponding to position 58 in SEQ ID NO: 3; D, E, R at a residue corresponding to position 61 in SEQ ID NO: 3; F, V, W at a residue corresponding to position 62 in SEQ ID NO: 3; D, E, Q at a residue corresponding to position 63 in SEQ ID NO: 3; H at a residue corresponding to position 64 in SEQ ID NO: 3; R at a residue corresponding to position 65 in SEQ ID NO: 3; V at a residue corresponding to position 66 in SEQ ID NO: 3; E at a residue corresponding to position 67 in SEQ ID NO: 3; M, R at a residue corresponding to position 70 in SEQ ID NO: 3; D, E, Y, F, H at a residue corresponding to position 71 in SEQ ID NO: 3; T at a residue corresponding to position 73 in SEQ ID NO: 3; E, R at a residue corresponding to position 74 in SEQ ID NO: 3; G at a residue corresponding to position 75 in SEQ ID NO: 3; Y at a residue corresponding to position 77 in SEQ ID NO: 3; E, F, Y, H, N at a residue corresponding to position 78 in SEQ ID NO: 3; D, W at a residue corresponding to position 79 in SEQ ID NO: 3; F, V, I at a residue corresponding to position 80 in SEQ ID NO: 3; A, R at a residue corresponding to position 81 in SEQ ID NO: 3; G at a residue corresponding to position 82 in SEQ ID NO: 3; E at a residue corresponding to position 83 in SEQ ID NO: 3; R, Q at a residue corresponding to position 84 in SEQ ID NO: 3; V, K at a residue corresponding to position 86 in SEQ ID NO: 3; M at a residue corresponding to position 88 in SEQ ID NO: 3; V, L at a residue corresponding to position 89 in SEQ ID NO: 3; S, D, H, E, R at a residue corresponding to position 90 in SEQ ID NO: 3; E, Q at a residue corresponding to position 91 in SEQ ID NO: 3; D at a residue corresponding to position 92 in SEQ ID NO: 3; D at a residue corresponding to position 93 in SEQ ID NO: 3; G, D at a residue corresponding to position 94 in SEQ ID NO: 3; K, M, R at a residue corresponding to position 95 in SEQ ID NO: 3; N at a residue corresponding to position 96 in SEQ ID NO: 3; I, V, M at a residue corresponding to position 97 in SEQ ID NO: 3; Y, I, M, T at a residue corresponding to position 98 in SEQ ID NO: 3; R, T, Q, K at a residue corresponding to position 99 in SEQ ID NO: 3; Y, I, M, L at a residue corresponding to position 100 in SEQ ID NO: 3; Y, H at a residue corresponding to position 101 in SEQ ID NO: 3; R, Q at a residue corresponding to position 102 in SEQ ID NO: 3; F, M at a residue corresponding to position 104 in SEQ ID NO: 3; C, V at a residue corresponding to position 107 in SEQ ID NO: 3; T, S at a residue corresponding to position 111 in SEQ ID NO: 3; I, T, L at a residue corresponding to position 117 in SEQ ID NO: 3; T, S at a residue corresponding to position 119 in SEQ ID NO: 3; A, T, M at a residue corresponding to position 120 in SEQ ID NO: 3; L, V, I at a residue corresponding to position 121 in SEQ ID NO: 3; H at a residue corresponding to position 122 in SEQ ID NO: 3; I, V, L, M at a residue corresponding to position 124 in SEQ ID NO: 3; I, L, V at a residue corresponding to position 125 in SEQ ID NO: 3; A at a residue corresponding to position 126 in SEQ ID NO: 3; I at a residue corresponding to position 128 in SEQ ID NO: 3; A, M at a residue corresponding to position 129 in SEQ ID NO: 3; A, S at a residue corresponding to position 130 in SEQ ID NO: 3; C, T at a residue corresponding to position 133 in SEQ ID NO: 3; L, I at a residue corresponding to position 136 in SEQ ID NO: 3; R at a residue corresponding to position 144 in SEQ ID NO: 3; C, T, V at a residue corresponding to position 145 in SEQ ID NO: 3; N at a residue corresponding to position 146 in SEQ ID NO: 3; A, I, M at a residue corresponding to position 147 in SEQ ID NO: 3; E, L, A, R at a residue corresponding to position 148 in SEQ ID NO: 3; M, V, L, I at a residue corresponding to position 149 in SEQ ID NO: 3; A, I, M at a residue corresponding to position 150 in SEQ ID NO: 3; K, D, Q at a residue corresponding to position 151 in SEQ ID NO: 3; M, I at a residue corresponding to position 152 in SEQ ID NO: 3; M, C, F, V at a residue corresponding to position 153 in SEQ ID NO: 3; Q, K at a residue corresponding to position 154 in SEQ ID NO: 3; K, R, D, Q at a residue corresponding to position 155 in SEQ ID NO: 3; I at a residue corresponding to position 156 in SEQ ID NO: 3; A, F, I at a residue corresponding to position 157 in SEQ ID NO: 3; E, V, K at a residue corresponding to position 158 in SEQ ID NO: 3; D, K at a residue corresponding to position 159 in SEQ ID NO: 3; V, A at a residue corresponding to position 160 in SEQ ID NO: 3; F, Y, V, A, L at a residue corresponding to position 162 in SEQ ID NO: 3; A, D, P at a residue corresponding to position 164 in SEQ ID NO: 3; D, H at a residue corresponding to position 165 in SEQ ID NO: 3; V, C, I at a residue corresponding to position 166 in SEQ ID NO: 3; L, V, F at a residue corresponding to position 167 in SEQ ID NO: 3; T, N at a residue corresponding to position 168 in SEQ ID NO: 3; C, L, M, I at a residue corresponding to position 169 in SEQ ID NO: 3; V at a residue corresponding to position 170 in SEQ ID NO: 3; D, E, P, N at a residue corresponding to position 171 in SEQ ID NO: 3; D, G, H at a residue corresponding to position 172 in SEQ ID NO: 3; T at a residue corresponding to position 174 in SEQ ID NO: 3; I, H at a residue corresponding to position 175 in SEQ ID NO: 3; D at a residue corresponding to position 176 in SEQ ID NO: 3; A, T, I at a residue corresponding to position 177 in SEQ ID NO: 3; E at a residue corresponding to position 180 in SEQ ID NO: 3; I at a residue corresponding to position 181 in SEQ ID NO: 3; Q, A, H, K at a residue corresponding to position 183 in SEQ ID NO: 3; G, S at a residue corresponding to position 184 in SEQ ID NO: 3; M, V, I, F, K at a residue corresponding to position 187 in SEQ ID NO: 3; V, L at a residue corresponding to position 189 in SEQ ID NO: 3; A at a residue corresponding to position 190 in SEQ ID NO: 3; A, T, R, K at a residue corresponding to position 194 in SEQ ID NO: 3; V at a residue corresponding to position 196 in SEQ ID NO: 3; I at a residue corresponding to position 197 in SEQ ID NO: 3; R at a residue corresponding to position 198 in SEQ ID NO: 3; V at a residue corresponding to position 199 in SEQ ID NO: 3; C at a residue corresponding to position 200 in SEQ ID NO: 3; A at a residue corresponding to position 201 in SEQ ID NO: 3; A, T, R at a residue corresponding to position 202 in SEQ ID NO: 3; N, H, A at a residue corresponding to position 203 in SEQ ID NO: 3; V at a residue corresponding to position 206 in SEQ ID NO: 3; V, T, C at a residue corresponding to position 207 in SEQ ID NO: 3; V, M, Y at a residue corresponding to position 208 in SEQ ID NO: 3; A, P at a residue corresponding to position 212 in SEQ ID NO: 3; S at a residue corresponding to position 214 in SEQ ID NO: 3; P, N, C, A, S at a residue corresponding to position 215 in SEQ ID NO: 3; A, P at a residue corresponding to position 216 in SEQ ID NO: 3; M, A, C at a residue corresponding to position 217 in SEQ ID NO: 3; I, V at a residue corresponding to position 218 in SEQ ID NO: 3; I, M, L at a residue corresponding to position 219 in SEQ ID NO: 3; P, D at a residue corresponding to position 221 in SEQ ID NO: 3; T, S at a residue corresponding to position 222 in SEQ ID NO: 3; C at a residue corresponding to position 223 in SEQ ID NO: 3; N at a residue corresponding to position 224 in SEQ ID NO: 3; V at a residue corresponding to position 225 in SEQ ID NO: 3; M, E, K at a residue corresponding to position 227 in SEQ ID NO: 3; A at a residue corresponding to position 228 in SEQ ID NO: 3; V at a residue corresponding to position 229 in SEQ ID NO: 3; D, E, P, K at a residue corresponding to position 230 in SEQ ID NO: 3; R, Q, I, K at a residue corresponding to position 231 in SEQ ID NO: 3; M at a residue corresponding to position 232 in SEQ ID NO: 3; L at a residue corresponding to position 233 in SEQ ID NO: 3; F, A, W, M, Q at a residue corresponding to position 234 in SEQ ID NO: 3; F, Y, C, M at a residue corresponding to position 237 in SEQ ID NO: 3; R, Q, M at a residue corresponding to position 238 in SEQ ID NO: 3; T at a residue corresponding to position 240 in SEQ ID NO: 3; V at a residue corresponding to position 243 in SEQ ID NO: 3; I at a residue corresponding to position 245 in SEQ ID NO: 3; S, A at a residue corresponding to position 246 in SEQ ID NO: 3; N at a residue corresponding to position 248 in SEQ ID NO: 3; Y at a residue corresponding to position 249 in SEQ ID NO: 3; M at a residue corresponding to position 250 in SEQ ID NO: 3; F, L at a residue corresponding to position 251 in SEQ ID NO: 3; V, I at a residue corresponding to position 252 in SEQ ID NO: 3; H at a residue corresponding to position 253 in SEQ ID NO: 3; K, R, D at a residue corresponding to position 254 in SEQ ID NO: 3; E at a residue corresponding to position 255 in SEQ ID NO: 3; I, M, V, R at a residue corresponding to position 256 in SEQ ID NO: 3; Y, A at a residue corresponding to position 257 in SEQ ID NO: 3; D at a residue corresponding to position 258 in SEQ ID NO: 3; R, A, Q, D at a residue corresponding to position 259 in SEQ ID NO: 3; V, L at a residue corresponding to position 260 in SEQ ID NO: 3; V, L, M at a residue corresponding to position 261 in SEQ ID NO: 3; K at a residue corresponding to position 262 in SEQ ID NO: 3; K, R at a residue corresponding to position 263 in SEQ ID NO: 3; V, F at a residue corresponding to position 264 in SEQ ID NO: 3; R, Q, E at a residue corresponding to position 265 in SEQ ID NO: 3; E at a residue corresponding to position 266 in SEQ ID NO: 3; R at a residue corresponding to position 267 in SEQ ID NO: 3; F, L, W, M, I at a residue corresponding to position 270 in SEQ ID NO: 3; W, F, K, H at a residue corresponding to position 271 in SEQ ID NO: 3; V, L, M at a residue corresponding to position 272 in SEQ ID NO: 3; C at a residue corresponding to position 273 in SEQ ID NO: 3; D, E at a residue corresponding to position 274 in SEQ ID NO: 3; A, P, G, E at a residue corresponding to position 275 in SEQ ID NO: 3; R, D at a residue corresponding to position 276 in SEQ ID NO: 3; E, Q at a residue corresponding to position 277 in SEQ ID NO: 3; E at a residue corresponding to position 278 in SEQ ID NO: 3; S, E at a residue corresponding to position 279 in SEQ ID NO: 3; T at a residue corresponding to position 280 in SEQ ID NO: 3; I, V, M at a residue corresponding to position 281 in SEQ ID NO: 3; K, Q, G, M at a residue corresponding to position 282 in SEQ ID NO: 3; E, N, H at a residue corresponding to position 283 in SEQ ID NO: 3; V at a residue corresponding to position 284 in SEQ ID NO: 3; V at a residue corresponding to position 285 in SEQ ID NO: 3; Y, V, I at a residue corresponding to position 286 in SEQ ID NO: 3; E at a residue corresponding to position 288 in SEQ ID NO: 3; H at a residue corresponding to position 290 in SEQ ID NO: 3; L, A, T, H, N, K at a residue corresponding to position 291 in SEQ ID NO: 3; A, I, N at a residue corresponding to position 293 in SEQ ID NO: 3; R at a residue corresponding to position 294 in SEQ ID NO: 3; D, R, Q at a residue corresponding to position 295 in SEQ ID NO: 3; L, W at a residue corresponding to position 296 in SEQ ID NO: 3; K, Q, R at a residue corresponding to position 299 in SEQ ID NO: 3; A at a residue corresponding to position 300 in SEQ ID NO: 3; A at a residue corresponding to position 301 in SEQ ID NO: 3; A, E, Q at a residue corresponding to position 302 in SEQ ID NO: 3; E, R at a residue corresponding to position 303 in SEQ ID NO: 3; L at a residue corresponding to position 304 in SEQ ID NO: 3; V at a residue corresponding to position 305 in SEQ ID NO: 3; E, H, K, R at a residue corresponding to position 306 in SEQ ID NO: 3; I, M, V at a residue corresponding to position 307 in SEQ ID NO: 3; T at a residue corresponding to position 308 in SEQ ID NO: 3; F, V, L at a residue corresponding to position 310 in SEQ ID NO: 3; D, S, R, E at a residue corresponding to position 311 in SEQ ID NO: 3; A, T, D, K at a residue corresponding to position 312 in SEQ ID NO: 3; V, L at a residue corresponding to position 313 in SEQ ID NO: 3; D at a residue corresponding to position 315 in SEQ ID NO: 3; G, E, N at a residue corresponding to position 316 in SEQ ID NO: 3; G, T at a residue corresponding to position 317 in SEQ ID NO: 3; R at a residue corresponding to position 318 in SEQ ID NO: 3; L, M at a residue corresponding to position 319 in SEQ ID NO: 3; Y, L at a residue corresponding to position 320 in SEQ ID NO: 3; I, L, C at a residue corresponding to position 321 in SEQ ID NO: 3; G, V, A, T at a residue corresponding to position 322 in SEQ ID NO: 3; I, V, K, P at a residue corresponding to position 323 in SEQ ID NO: 3; I, V, L at a residue corresponding to position 324 in SEQ ID NO: 3; E, T at a residue corresponding to position 325 in SEQ ID NO: 3; D at a residue corresponding to position 326 in SEQ ID NO: 3; F, W, V at a residue corresponding to position 327 in SEQ ID NO: 3; S at a residue corresponding to position 328 in SEQ ID NO: 3; P, R, D, N at a residue corresponding to position 329 in SEQ ID NO: 3; E at a residue corresponding to position 330 in SEQ ID NO: 3; D at a residue corresponding to position 331 in SEQ ID NO: 3; T, R at a residue corresponding to position 332 in SEQ ID NO: 3; I, M, F at a residue corresponding to position 333 in SEQ ID NO: 3; S, V, C, M at a residue corresponding to position 334 in SEQ ID NO: 3; R at a residue corresponding to position 335 in SEQ ID NO: 3; M at a residue corresponding to position 339 in SEQ ID NO: 3; I at a residue corresponding to position 341 in SEQ ID NO: 3; I, M at a residue corresponding to position 342 in SEQ ID NO: 3; S, C, V at a residue corresponding to position 343 in SEQ ID NO: 3; M, I, V, L at a residue corresponding to position 344 in SEQ ID NO: 3; L, M, Y, V at a residue corresponding to position 345 in SEQ ID NO: 3; P at a residue corresponding to position 346 in SEQ ID NO: 3; F, V at a residue corresponding to position 347 in SEQ ID NO: 3; E, K, S at a residue corresponding to position 348 in SEQ ID NO: 3; G, T, N, S, D at a residue corresponding to position 349 in SEQ ID NO: 3; E, I, V, F, L at a residue corresponding to position 350 in SEQ ID NO: 3; D at a residue corresponding to position 351 in SEQ ID NO: 3; L, C at a residue corresponding to position 354 in SEQ ID NO: 3; D, A, Q, S, E at a residue corresponding to position 355 in SEQ ID NO: 3; L, V at a residue corresponding to position 356 in SEQ ID NO: 3; R, N at a residue corresponding to position 358 in SEQ ID NO: 3; S, A, N, E at a residue corresponding to position 359 in SEQ ID NO: 3; S at a residue corresponding to position 360 in SEQ ID NO: 3; S, N, T at a residue corresponding to position 362 in SEQ ID NO: 3; E at a residue corresponding to position 363 in SEQ ID NO: 3; A, K, Y, L at a residue corresponding to position 366 in SEQ ID NO: 3; C, A at a residue corresponding to position 370 in SEQ ID NO: 3; S, A at a residue corresponding to position 371 in SEQ ID NO: 3; V, M at a residue corresponding to position 372 in SEQ ID NO: 3; F, Y, W at a residue corresponding to position 373 in SEQ ID NO: 3; T at a residue corresponding to position 374 in SEQ ID NO: 3; H, T, K, R, Q at a residue corresponding to position 375 in SEQ ID NO: 3; D at a residue corresponding to position 376 in SEQ ID NO: 3; E, I, V at a residue corresponding to position 377 in SEQ ID NO: 3; K, A, D at a residue corresponding to position 378 in SEQ ID NO: 3; R, N, V at a residue corresponding to position 379 in SEQ ID NO: 3; V at a residue corresponding to position 380 in SEQ ID NO: 3; W, H, M, R, K, T at a residue corresponding to position 381 in SEQ ID NO: 3; K at a residue corresponding to position 382 in SEQ ID NO: 3; I, M at a residue corresponding to position 383 in SEQ ID NO: 3; G, S at a residue corresponding to position 384 in SEQ ID NO: 3; R, K at a residue corresponding to position 385 in SEQ ID NO: 3; Q, K, R at a residue corresponding to position 386 in SEQ ID NO: 3; M, V, L at a residue corresponding to position 387 in SEQ ID NO: 3; R, E, Q, H, D at a residue corresponding to position 388 in SEQ ID NO: 3; T, A, C at a residue corresponding to position 389 in SEQ ID NO: 3; G, T, C at a residue corresponding to position 390 in SEQ ID NO: 3; M, V, I at a residue corresponding to position 392 in SEQ ID NO: 3; G at a residue corresponding to position 393 in SEQ ID NO: 3; I at a residue corresponding to position 394 in SEQ ID NO: 3; E, D, S at a residue corresponding to position 396 in SEQ ID NO: 3; W, I, Y at a residue corresponding to position 397 in SEQ ID NO: 3; S, I, H at a residue corresponding to position 400 in SEQ ID NO: 3; D, G, S, H, A at a residue corresponding to position 401 in SEQ ID NO: 3; M at a residue corresponding to position 402 in SEQ ID NO: 3; V, G at a residue corresponding to position 405 in SEQ ID NO: 3; W, F, L at a residue corresponding to position 406 in SEQ ID NO: 3; H, Q at a residue corresponding to position 407 in SEQ ID NO: 3; M at a residue corresponding to position 410 in SEQ ID NO: 3; G, A, P, T at a residue corresponding to position 411 in SEQ ID NO: 3; V at a residue corresponding to position 414 in SEQ ID NO: 3; S at a residue corresponding to position 420 in SEQ ID NO: 3; F at a residue corresponding to position 421 in SEQ ID NO: 3; G, R at a residue corresponding to position 423 in SEQ ID NO: 3; I, A, G at a residue corresponding to position 425 in SEQ ID NO: 3; V at a residue corresponding to position 426 in SEQ ID NO: 3; A, T at a residue corresponding to position 427 in SEQ ID NO: 3; D at a residue corresponding to position 428 in SEQ ID NO: 3; N at a residue corresponding to position 431 in SEQ ID NO: 3; T, H, K, M, R at a residue corresponding to position 433 in SEQ ID NO: 3; M, V at a residue corresponding to position 435 in SEQ ID NO: 3; V, F, I at a residue corresponding to position 436 in SEQ ID NO: 3; Q at a residue corresponding to position 437 in SEQ ID NO: 3; I, T, Y, C at a residue corresponding to position 438 in SEQ ID NO: 3; S at a residue corresponding to position 439 in SEQ ID NO: 3; W, K at a residue corresponding to position 440 in SEQ ID NO: 3; M, I, L at a residue corresponding to position 441 in SEQ ID NO: 3; W, F, R at a residue corresponding to position 443 in SEQ ID NO: 3; K at a residue corresponding to position 444 in SEQ ID NO: 3; K at a residue corresponding to position 446 in SEQ ID NO: 3; E, N at a residue corresponding to position 447 in SEQ ID NO: 3; W, K, P at a residue corresponding to position 448 in SEQ ID NO: 3; R, H at a residue corresponding to position 449 in SEQ ID NO: 3; V at a residue corresponding to position 450 in SEQ ID NO: 3; S, K at a residue corresponding to position 452 in SEQ ID NO: 3; H, P at a residue corresponding to position 453 in SEQ ID NO: 3; A, D at a residue corresponding to position 454 in SEQ ID NO: 3; K, D at a residue corresponding to position 455 in SEQ ID NO: 3; F at a residue corresponding to position 457 in SEQ ID NO: 3; H, G at a residue corresponding to position 458 in SEQ ID NO: 3; D at a residue corresponding to position 459 in SEQ ID NO: 3; more than one of the foregoing; or any combination thereof.

In some embodiments, provided herein is microbial organism (e.g., host microbial organism) that has a recombinant nucleic acid encoding an engineered ALDH having: V at a residue corresponding to position 42 in SEQ ID NO: 3; T or S at a residue corresponding to position 119 in SEQ ID NO: 3; I at a residue corresponding to position 149 in SEQ ID NO: 3; K at a residue corresponding to position 158 in SEQ ID NO: 3; T at a residue corresponding to position 174 in SEQ ID NO: 3; A at a residue corresponding to position 246 in SEQ ID NO: 3; D at a residue corresponding to position 259 in SEQ ID NO: 3; R at a residue corresponding to position 263 in SEQ ID NO: 3; R at a residue corresponding to position 299 in SEQ ID NO: 3; V at a residue corresponding to position 327 in SEQ ID NO: 3; S at a residue corresponding to position 348 in SEQ ID NO: 3; A at a residue corresponding to position 370 in SEQ ID NO: 3; I at a residue corresponding to position 392 in SEQ ID NO: 3; A at a residue corresponding to position 401 in SEQ ID NO: 3; T at a residue corresponding to position 427 in SEQ ID NO: 3; more than one of the foregoing; or any combination thereof.

In some embodiments, provided herein is microbial organism (e.g., host microbial organism) that has a recombinant nucleic acid encoding an engineered ALDH having one or more amino acid alterations comprising: E2P, E2Q, E2T, Q3E, Q3K, Q3M, Q3R, Q3S, A4G, A4S, V5I, V5T, K6E, K6F, K6H, K6P, K6Q, K6R, D7E, D7Q, Y8D, L9I, L9V, D10N, K11A, K11D, K11E, K11Q, K11R, M12A, M12I, M12L, V13L, A14D, A14E, A14K, A14Q, A15K, A15R, S16A, R17H, R17K, R17Q, 118E, I18K, I18Q, I18T, Q20F, Q20H, Q20M, Q21A, Q21E, Q21K, Q21P, Q21R, E22A, E22K, E22Q, F23L, F23W, F23Y, A24E, A24K, A24R, A24S, T25A, T25E, T25K, T25N, T25S, Y26F, Y26L, Y26M, Y26T, Y26W, P27D, P27S, P27T, Q28W, E29K, E29Q, T30D, T30E, T30K, T30Q, T30R, V31I, V31L, V31R, D32A, D32E, D32N, K33A, K33E, K33Q, K33R, A34I, A34L, A34M, A34V, V35C, V35I, V35L, V35M, R36E, R36H, R36K, T37A, T37K, T37R, V38F, V38I, V38L, V38M, V38W, G39A, G39R, K40D, K40E, K40Q, K40R, K40W, A41L, A41V, I42C, I42L, I42M, I42V, Y43E, Y43L, Y43R, D44E, D44K, D44Q, D44R, N45H, N45Q, N45R, E47D, L48D, L48E, L48M, L48R, L49F, L49I, L49M, L49W, L49Y, A50G, H51A, H51E, H51K, H51N, H51Q, H51R, M52L, A53E, A53I, A53S, V54I, V54L, V54M, V54T, D55E, D55Q, D55R, K58E, K58G, K58Q, K58R, M59F, M59I, M59L, M59Y, G60A, N61D, N61E, N61K, N61R, Y62A, Y62F, Y62I, Y62M, Y62V, Y62W, A63D, A63E, A63Q, D64E, D64H, K65E, K65R, I66L, I66V, A67E, K68D, K68E, C69H, C69I, C69L, C69M, C69N, V70I, V70L, V70M, V70R, N71D, N71E, N71F, N71H, N71Y, K72A, K72R, S73A, S73T, K74D, K74E, K74R, S75G, S75P, S75Y, V76F, V76I, V76L, V76M, V76Y, W77F, W77Y, W78E, W78F, W78H, W78N, W78R, W78Y, R79D, R79S, R79W, R79Y, M80F, M80I, M80L, M80V, K81A, K81E, K81R, D82E, D82G, D82P, K83E, K83Q, K84Q, K84R, S85A, S85T, R86E, R86K, R86V, G87T, I88L, I88M, I88V, I89L, I89P, I89V, K90D, K90E, K90H, K90P, K90R, K90S, R91D, R91E, R91Q, I92D, I92M, P93D, P93E, E94A, E94D, E94G, E94N, L95K, L95M, L95N, L95R, G96H, G96K, G96N, G96R, L97I, L97M, L97V, V98I, V98M, V98T, V98Y, E99K, E99Q, E99R, E99T, V100Y, V100L, V100M, V100I, A101K, A101Y, A101R, A101H, K102E, K102Q, K102R, K102V, I104V, I104Y, I104F, I104L, I104M, V106I, I107C, I107V, G108A, G108C, C109I, C109A, C109M, V110M, V110L, V110I, A111T, A111V, A111Y, A111I, A111S, P112E, T113F, T113S, T113W, T113Y, T114N, N115W, N115R, N115H, N115Y, V117N, V117L, V117T, V117I, I118N, I118A, I118L, I118M, I118S, I118F, I118V, N119Q, N119I, N119L, N119M, N119T, N119S, V120T, V120A, V120P, V120M, M121V, M121A, M121L, M121C, M121I, Q122C, Q122F, Q122R, Q122Y, Q122H, Q122N, Q122S, Q122G, Q122V, Q122W, N123K, N123P, A124V, A124L, A124M, A124I, M125I, M125L, M125A, M125V, C126A, C126P, A127M, L128I, K129A, K129M, C130T, C130S, C130A, G131R, N132C, S133T, S133C, M134I, M134C, M134V, I135V, I135M, V136L, V136M, V136F, V136I, S137R, S137K, P138G, H139S, H139G, P140E, R141Q, R141K, R141Y, R141E, A142T, A142S, K143P, H144L, H144N, H144R, S145T, S145C, S145V, S146T, S146C, S146A, S146N, V147I, V147M, V147A, K148Y, K148L, K148A, K148E, K148H, K148R, T149V, T149I, T149L, T149M, T149F, V150I, V150M, V150G, V150A, E151D, E151K, E151R, E151Q, V152L, V152M, V152I, I153F, I153V, I153C, I153M, N154H, N154A, N154Q, N154K, N154R, E155R, E155K, E155D, E155Q, A156I, L157A, L157I, L157F, A158E, A158V, A158K, A159K, A159Q, A159D, A159E, L160V, L160C, L160A, M162I, M162A, M162F, M162Y, M162L, M162V, K164D, K164A, K164P, N165H, N165G, N165D, L166C, L166A, L166I, L166V, I167V, I167F, I167L, Q168T, Q168N, V169L, V169W, V169C, V169M, V169I, I170L, I170V, T171E, T171P, T171N, T171D, E172D, E172G, E172H, P173G, P173D, S174G, S174D, S174T, M175I, M175H, M175R, E176Q, E176D, L177V, L177T, L177A, L177I, S178V, S178A, S178T, S178G, A179D, A179N, A179E, G180H, G180Q, G180A, G180E, L181I, L181F, M182L, M182H, M182I, M182V, S183Q, S183K, S183D, S183A, S183H, S183E, A184H, A184P, A184S, A184G, V185C, V185I, D186R, L187H, L187K, L187F, L187I, L187V, L187M, C188I, C188L, C188V, I189S, I189F, I189T, I189M, I189V, I189L, C190P, C190A, C190F, T191R, G193S, P194T, P194R, P194K, P194A, G195A, L196M, L196I, L196V, V197I, V197M, V197G, V197A, K198Q, K198E, K198R, A199S, A199V, A200C, A200I, A200V, Y201M, Y201Q, Y201A, S202N, S202K, S202A, S202R, S202T, S202E, S202Q, S203N, S203H, S203A, K205R, K205T, P206V, P206R, P206K, A207V, A207S, A207C, A207T, I208V, I208T, I208Y, I208M, I208L, G209L, G209E, G209M, V210H, V210L, G211L, Q212A, Q212P, Q212G, G213K, N214S, N214K, V215S, V215N, V215A, V215C, V215D, V215P, Q216H, Q216A, Q216P, V217F, V217A, V217I, V217C, V217M, L218I, L218V, L218F, L218Y, V219M, V219F, V219L, V219I, D220C, D220F, R221D, R221P, D222T, D222S, A223C, D224N, L225V, D226E, Q227K, Q227R, Q227M, Q227E, V228A, A229W, A229V, A230R, A230D, A230P, A230Q, A230K, A230E, M231Q, M231R, M231I, M231K, V232I, V232F, V232T, V232C, V232Q, V232M, V232N, I233V, I233L, I233M, K234W, K234Q, K234F, K234A, K234M, G235A, G235S, R236K, R236C, R236W, R236A, R236I, T237F, T237Y, T237C, T237M, F238R, F238M, F238Q, F238Y, F238W, D239R, D239H, D239Q, N240Q, N240A, N240T, N240C, N240S, G241N, L243V, L243M, L243T, L243C, T245M, T245I, T245N, T245S, T245V, T245A, T245C, C246S, C246A, E247G, E247P, E247I, E247A, Q248D, Q248T, Q248E, Q248K, Q248S, Q248N, N249Y, N249R, V250I, V250L, V250M, I251Y, I251F, I251L, I251V, C252V, C252I, P253D, P253Q, P253H, P253V, P253E, E254Q, E254K, E254R, E254D, D255E, K256V, K256M, K256I, K256R, K257A, K257F, K257H, K257Y, E258D, E259A, E259R, E259K, E259D, E259Q, M260I, M260V, M260F, M260L, I261L, I261M, I261V, A262Q, A262D, A262E, A262K, A263E, A263K, A263R, L264M, L264V, L264F, K265Q, K265R, K265A, K265E, K266A, K266E, H267Y, H267R, H267N, H267A, H267Q, G268A, A269S, A269G, A269C, Y270L, Y270F, Y270I, Y270M, Y270W, Y271F, Y271K, Y271V, Y271W, Y271R, Y271H, I272V, I272M, I272L, G273C, N274E, N274D, S275P, S275G, S275A, S275E, E276Q, E276M, E276R, E276D, D277Q, D277E, A278P, A278E, A279E, A279D, A279S, K280Q, K280R, K280T, L281V, L281M, L281I, R282Q, R282K, R282M, R282G, R282E, D283E, D283H, D283N, T284V, T284P, A285L, A285V, A285I, A285M, F286L, F286I, F286W, F286Y, F286V, N288R, N288D, N288E, G289M, G290H, P291K, P291L, P291E, P291T, P291H, P291A, P291N, V292I, V292M, S293A, S293V, S293I, S293N, K294S, K294N, K294P, K294E, K294R, E295R, E295K, E295A, E295Q, E295D, Y296W, Y296R, Y296V, Y296F, Y296H, Y296L, P297A, P297T, P297Q, P297V, P297L, P297H, P297I, G298A, A299K, A299Q, A299R, S300A, S300R, V301I, V301A, K302A, K302R, K302D, K302Q, K302E, K303Q, K303W, K303R, K303E, I304L, I304G, I304Y, A305I, A305V, Q306H, Q306K, Q306E, Q306R, L307I, L307V, L307M, S308A, S308T, I310G, I310V, I310L, I310F, Q311R, Q311T, Q311K, Q311S, Q311D, Q311E, G312A, G312D, G312T, G312K, I313L, I313V, P314A, E315D, D316E, D316G, D316N, A317G, A317T, K318R, K318T, K318Y, V319F, V319L, V319M, V319I, V319Y, I320Y, I320L, V321L, V321P, V321C, V321I, S322A, S322T, S322G, S322V, C323E, C323L, C323I, C323G, C323P, C323V, C323K, T324I, T324V, T324L, R325K, R325E, R325D, R325T, G326D, Y327W, Y327F, Y327V, G328D, G328T, G328S, K329E, K329R, K329P, K329D, K329N, D330E, E331S, E331D, E331H, E331M, E331Y, P332R, P332T, L333M, L333V, L333F, L333I, A334V, A334S, A334M, A334C, K335R, K335Q, K335H, K337Q, K337E, L338F, L338V, L338I, L338M, F339C, F339M, V341I, L342M, L342V, L342I, A343C, A343T, A343V, A343S, A343P, F344I, F344V, F344L, F344M, F345Y, F345V, F345M, F345L, F345I, T346R, T346K, T346P, Y347F, Y347V, D348E, D348K, D348S, K349S, K349G, K349D, K349N, K349T, W350V, W350E, W350I, W350L, W350F, E351D, D352Q, D352H, D352E, I354L, I354V, I354C, H355R, H355A, H355D, H355Q, H355S, H355E, I356M, I356L, I356V, A357C, K358N, K358R, T359A, T359D, T359S, T359N, T359E, N360S, N360T, L361D, L361V, E362L, E362T, E362N, E362S, M363Y, M363E, E364Q, E364Y, E364F, E364H, I366Y, I366K, I366F, I366A, I366L, H368L, H368A, S369T, S369A, V370C, V370A, V371M, V371I, V371A, V371W, V371S, V371Y, I372V, I372M, H373W, H373Y, H373F, S374T, N375H, N375Q, N375K, N375T, N375R, T376N, T376S, T376D, P377D, P377I, P377V, P377E, E378K, E378D, E378A, H379N, H379K, H379V, H379R, I380A, I380M, I380V, E381M, E381W, E381K, E381R, E381T, E381H, A382K, A382R, A382E, A382Q, V383M, V383L, V383Y, V383F, V383I, A384S, A384G, E385Q, E385L, E385R, E385K, A386E, A386Q, A386K, A386R, I387M, I387V, I387L, P388R, P388H, P388D, P388Q, P388E, V389A, V389C, V389T, S390T, S390N, S390C, S390G, R391M, R391T, R391I, F392V, F392I, F392M, A393M, A393I, A393W, A393L, A393G, V394I, V394K, Q396R, Q396E, Q396S, Q396D, V397I, V397W, V397A, V397P, V397F, V397Y, G398S, G398C, G398P, G398T, G399A, G399S, T400I, T400S, T400F, T400V, T400H, N401G, N401H, N401D, N401S, N401A, L402N, L402A, L402F, L402M, G403S, A405I, A405F, A405V, A405G, M406Y, M406K, M406F, M406L, M406W, D407H, D407Q, D407Y, D407E, N408T, N408S, N408H, L410F, L410I, L410M, N411T, N411G, N411P, N411A, P412F, P412R, T413A, T414M, T414F, T414V, T415P, T415S, L416M, L416F, L416I, C418F, C418I, C418T, T420S, T420R, W421Y, W421F, N423K, N423R, N423G, N424S, N424R, S425I, S425G, S425W, S425A, I426V, I426T, I426L, S427G, S427T, S427A, E428D, E428H, N429E, F430I, F430Y, F430L, F430V, T431G, T431S, T431N, Y432A, Y432P, Y432F, Y432W, Y433T, Y433R, Y433K, Y433H, Y433M, Y433E, H434R, H434Q, H434D, H434N, L435V, L435F, L435W, L435Y, L435M, M436T, M436L, M436F, M436I, M436V, N437H, N437Q, N437Y, L438V, L438T, L438W, L438C, L438Y, L438I, T439Q, T439S, T439R, T439K, R440K, R440W, V441L, V441I, V441M, S442G, S442A, Y443H, Y443W, Y443R, Y443F, R444E, R444P, R444K, V445I, V445Y, V445F, V445M, V445L, P446E, P446K, D447E, D447N, M448W, M448K, M448P, Y449E, Y449R, Y449F, Y449H, I450M, I450C, I450F, I450V, I450L, P451W, T452S, T452D, T452K, D453E, D453P, D453H, E454A, E454D, E455K, E455Q, E455D, I456F, I456L, I456V, I456W, I456M, W457M, W457Y, W457F, A458H, A458G, E459D, or any combination thereof.

In some embodiments, provided herein is microbial organism (e.g., host microbial organism) that has a recombinant nucleic acid encoding an engineered ALDH having one or more amino acid alterations comprising: E2Q, E2T, E2P, Q3S, Q3K, A4G, A4S, V5I, V5T, K6F, D7E, D7Q, L9V, K11E, K11D, K11Q, K11R, M12L, M12A, A15R, A15K, S16A, R17K, I18K, 118E, I18Q, Q20F, Q20H, Q21A, Q21R, E22K, E22Q, E22A, F23Y, A24E, A24K, T25A, T25E, Y26L, Y26M, Y26W, P27D, P27T, Q28W, T30D, T30K, T30Q, V31I, V31L, D32N, D32E, K33E, K33Q, K33A, K33R, A34V, V35C, V35M, R36K, T37K, T37A, T37R, V38L, V38I, G39A, K40W, A41L, A41V, I42L, I42V, I42M, I42C, Y43E, Y43L, D44K, D44Q, N45H, N45R, E47D, L48E, L48M, L48D, L49I, H51N, H51E, H51Q, M52L, V54L, D55E, D55R, K58R, N61D, N61E, N61R, Y62F, Y62V, Y62W, A63D, A63E, A63Q, D64H, K65R, I66V, A67E, V70M, V70R, N71D, N71E, N71Y, N71F, N71H, S73T, K74E, K74R, S75G, W77Y, W78E, W78F, W78Y, W78H, W78N, R79D, R79W, M80F, M80V, M80I, K81A, K81R, D82G, K83E, K84R, K84Q, R86V, R86K, I88M, I89V, I89L, K90S, K90D, K90H, K90E, K90R, R91E, R91Q, I92D, P93D, E94G, E94D, L95K, L95M, L95R, G96N, L97I, L97V, L97M, V98Y, V98I, V98M, V98T, E99R, E99T, E99Q, E99K, V100Y, V100I, V100M, V100L, A101Y, A101H, K102R, K102Q, I104F, I104M, I107C, I107V, A111T, A111S, V117I, V117T, V117L, N119T, N119S, V120A, V120T, V120M, M121L, M121V, M121I, Q122H, A124I, A124V, A124L, A124M, M125I, M125L, M125V, C126A, L128I, K129A, K129M, C130A, C130S, S133C, S133T, V136L, V136I, H144R, S145C, S145T, S145V, S146N, V147A, V147I, V147M, K148E, K148L, K148A, K148R, T149M, T149V, T149L, T149I, V150A, V150I, V150M, E151K, E151D, E151Q, V152M, V152I, I153M, I153C, I153F, I153V, N154Q, N154K, E155K, E155R, E155D, E155Q, A156I, L157A, L157F, L157I, A158E, A158V, A158K, A159D, A159K, L160V, L160A, M162F, M162Y, M162V, M162A, M162L, K164A, K164D, K164P, N165D, N165H, L166V, L166C, L166I, I167L, I167V, I167F, Q168T, Q168N, V169C, V169L, V169M, V169I, I170V, T171D, T171E, T171P, T171N, E172D, E172G, E172H, S174T, M175I, M175H, E176D, L177A, L177T, L177I, G180E, L181I, S183Q, S183A, S183H, S183K, A184G, A184S, L187M, L187V, L187I, L187F, L187K, I189V, I189L, C190A, P194A, P194T, P194R, P194K, L196V, V197I, K198R, A199V, A200C, Y201A, S202A, S202T, S202R, S203N, S203H, S203A, P206V, A207V, A207T, A207C, I208V, I208M, I208Y, Q212A, Q212P, N214S, V215P, V215N, V215C, V215A, V215S, Q216A, Q216P, V217M, V217A, V217C, L218I, L218V, V2191, V219M, V219L, R221P, R221D, D222T, D222S, A223C, D224N, L225V, Q227M, Q227E, Q227K, V228A, A229V, A230D, A230E, A230P, A230K, M231R, M231Q, M231I, M231K, V232M, I233L, K234F, K234A, K234W, K234M, K234Q, T237F, T237Y, T237C, T237M, F238R, F238Q, F238M, N240T, L243V, T245I, C246S, C246A, Q248N, N249Y, V250M, I251F, I251L, C252V, C252I, P253H, E254K, E254R, E254D, D255E, K256I, K256M, K256V, K256R, K257Y, K257A, E258D, E259R, E259A, E259Q, E259D, M260V, M260L, I261V, I261L, I261M, A262K, A263K, A263R, L264V, L264F, K265R, K265Q, K265E, K266E, H267R, Y270F, Y270L, Y270W, Y270M, Y270I, Y271W, Y271F, Y271K, Y271H, I272V, I272L, I272M, G273C, N274D, N274E, S275A, S275P, S275G, S275E, E276R, E276D, D277E, D277Q, A278E, A279S, A279E, K280T, L281I, L281V, L281M, R282K, R282Q, R282G, R282M, D283E, D283N, D283H, T284V, A285V, F286Y, F286V, F286I, N288E, G290H, P291L, P291A, P291T, P291H, P291N, P291K, S293A, S293I, S293N, K294R, E295D, E295R, E295Q, Y296L, Y296W, A299K, A299Q, A299R, S300A, V301A, K302A, K302E, K302Q, K303E, K303R, I304L, A305V, Q306E, Q306H, Q306K, Q306R, L307I, L307M, L307V, S308T, I310F, I310V, I310L, Q311D, Q311S, Q311R, Q311E, G312A, G312T, G312D, G312K, 1313V, I313L, E315D, D316G, D316E, D316N, A317G, A317T, K318R, V319L, V319M, I320Y, I320L, V321I, V321L, V321C, S322G, S322V, S322A, S322T, C323I, C323V, C323K, C323P, T324I, T324V, T324L, R325E, R325T, G326D, Y327F, Y327W, Y327V, G328S, K329P, K329R, K329D, K329N, D330E, E331D, P332T, P332R, L333I, L333M, L333F, A334S, A334V, A334C, A334M, K335R, F339M, V341I, L342I, L342M, A343S, A343C, A343V, F344M, F344I, F344V, F344L, F345L, F345M, F345Y, F345V, T346P, Y347F, Y347V, D348E, D348K, D348S, K349G, K349T, K349N, K349S, K349D, W350E, W350I, W350V, W350F, W350L, E351D, I354L, I354C, H355D, H355A, H355Q, H355S, H355E, I356L, I356V, K358R, K358N, T359S, T359A, T359N, T359E, N360S, E362S, E362N, E362T, M363E, I366A, I366K, I366Y, I366L, V370C, V370A, V371S, V371A, I372V, I372M, H373F, H373Y, H373W, S374T, N375H, N375T, N375K, N375R, N375Q, T376D, P377E, P377I, P377V, E378K, E378A, E378D, H379R, H379N, H379V, I380V, E381W, E381H, E381M, E381R, E381K, E381T, A382K, V383I, V383M, A384G, A384S, E385R, E385K, A386Q, A386K, A386R, I387M, I387V, I387L, P388R, P388E, P388Q, P388H, P388D, V389T, V389A, V389C, S390G, S390T, S390C, F392M, F392V, F392I, A393G, V394I, Q396E, Q396D, Q396S, V397W, V397I, V397Y, T400S, T400I, T400H, N401D, N401G, N401S, N401H, N401A, L402M, A405V, A405G, M406W, M406F, M406L, D407H, D407Q, L410M, N411G, N411A, N411P, N411T, T414V, T420S, W421F, N423G, N423R, S425I, S425A, S425G, I426V, S427A, S427T, E428D, T431N, Y433T, Y433H, Y433K, Y433M, Y433R, L435M, L435V, M436V, M436F, M4361, N437Q, L438I, L438T, L438Y, L438C, T439S, R440W, R440K, V441M, V441I, V441L, Y443W, Y443F, Y443R, R444K, P446K, D447E, D447N, M448W, M448K, M448P, Y449R, Y449H, I450V, T452S, T452K, D453H, D453P, E454A, E454D, E455K, E455D, W457F, A458H, A458G, or E459D.

In some embodiments, provided herein is microbial organism (e.g., host microbial organism) that has a recombinant nucleic acid encoding an engineered ALDH having one or more amino acid alterations comprising: I42V, N119T, N119S, T149I, A158K, S174T, C246A, E259D, A263R, A299R, Y327V, D348S, V370A, F392I, N401A, or S427T.

In some embodiments, provided herein is microbial organism (e.g., host microbial organism) that has a recombinant nucleic acid encoding an engineered ALDH provided herein has an amino acid sequence that is a variant of SEQ ID NO: 3 that includes one or more alterations as described in TABLE 12, wherein the portion, other than the one or more alterations described in TABLE 12, of the engineered ALDH has at least 65%, 70%, 75%, 80%, 85%, 90%, 95%, 98% or 99% sequence identity, or is identical, to an amino acid sequence referenced as SEQ ID NO: 3. Accordingly, in some embodiments, provided herein is microbial organism (e.g., host microbial organism) that has a recombinant nucleic acid encoding an engineered ALDH that has an amino acid sequence that includes one or more alterations as described in TABLE 12 and the portion, other than the alteration described in TABLE 12, of the engineered ALDH has at least 65% identical to SEQ ID NO: 3. In some embodiments, provided herein is microbial organism (e.g., host microbial organism) that has a recombinant nucleic acid encodes an engineered ALDH that has an amino acid sequence that includes one or more alterations as described in TABLE 12 and the portion, other than the alteration described in TABLE 12, of the engineered ALDH has at least 70% identical to SEQ ID NO: 3. In some embodiments, provided herein is microbial organism (e.g., host microbial organism) that has a recombinant nucleic acid encodes an engineered ALDH that has an amino acid sequence that includes one or more alterations as described in TABLE 12 and the portion, other than the alteration described in TABLE 12, of the engineered ALDH has at least 75% identical to SEQ ID NO: 3. In some embodiments, provided herein is microbial organism (e.g., host microbial organism) that has a recombinant nucleic acid encodes an engineered ALDH that has an amino acid sequence that includes one or more alterations as described in TABLE 12 and the portion, other than the alteration described in TABLE 12, of the engineered ALDH has at least 80% identical to SEQ ID NO: 3. In some embodiments, provided herein is microbial organism (e.g., host microbial organism) that has a recombinant nucleic acid encodes an engineered ALDH that has an amino acid sequence that includes one or more alterations as described in TABLE 12 and the portion, other than the alteration described in TABLE 12, of the engineered ALDH has at least 85% identical to SEQ ID NO: 3. In some embodiments, provided herein is microbial organism (e.g., host microbial organism) that has a recombinant nucleic acid encodes an engineered ALDH that has an amino acid sequence that includes one or more alterations as described in TABLE 12 and the portion, other than the alteration described in TABLE 12, of the engineered ALDH has at least 90% identical to SEQ ID NO: 3. In some embodiments, provided herein is microbial organism (e.g., host microbial organism) that has a recombinant nucleic acid encodes an engineered ALDH that has an amino acid sequence that includes one or more alterations as described in TABLE 12 and the portion, other than the alteration described in TABLE 12, of the engineered ALDH has at least 95% identical to SEQ ID NO: 3. In some embodiments, provided herein is microbial organism (e.g., host microbial organism) that has a recombinant nucleic acid encodes an engineered ALDH that has an amino acid sequence that includes one or more alterations as described in TABLE 12 and the portion, other than the alteration described in TABLE 12, of the engineered ALDH has at least 98% identical to SEQ ID NO: 3. In some embodiments, provided herein is microbial organism (e.g., host microbial organism) that has a recombinant nucleic acid encodes an engineered ALDH that has an amino acid sequence that includes one or more alterations as described in TABLE 12 and the portion, other than the alteration described in TABLE 12, of the engineered ALDH has at least 99% identical to SEQ ID NO: 3. In some embodiments, provided herein is microbial organism (e.g., host microbial organism) that has a recombinant nucleic acid encodes an engineered ALDH that has an amino acid sequence that includes one or more alterations as described in TABLE 12 and the portion, other than the alteration described in TABLE 12, of the engineered ALDH is identical to SEQ ID NO: 3.

In one embodiment, the cell comprises a pathway that produces 6-HH, HDO, an ester or amide of HDO, 6ACA-SADH, and/or HM4D. In one embodiment, the cell is capable of fermentation. In one embodiment, the cell further includes at least one substrate for the engineered ALDH described herein present or produced in the cell. In some embodiments, the substrate is HHA-CoA. In some embodiments, the substrate is 6ACA-CoA. In some embodiments, the cell has higher activity for HHA-CoA over adipyl-CoA, acetyl-CoA, succinyl-CoA, and/or 3-hydroxyadipyl-CoA. In some embodiments, the cell has higher activity for 6ACA-CoA over adipyl-CoA, acetyl-CoA, succinyl-CoA, and/or 3-hydroxyadipyl-CoA.

Also provided herein is a culture medium comprising a cell, or a population of cells as described herein.

The engineered ACL described herein can be utilized in a pathway that converts an acid to an acyl-CoA. Likewise, the engineered ALDH described herein can be utilized in a pathway that converts an acyl-CoA to its corresponding aldehyde. Exemplary pathways for HHA-CoA, 6-HH 1,6 HDO, 6ACA-CoA, 6ACA-SADH, and/or HMD that comprise an ACL and/or an ALDH aldehyde dehydrogenase have been described, for example, in US 2019/0300919, US 2007/0254341, US 2011/0091944, US 2011/0171699, WO 2009/151728, WO 2010/129936, and WO 2021/216952, all of which are incorporated herein by reference.

### Pathways, By-Products, Products

Exemplary HHA, HHA-CoA, 6-HH, and/or HDO pathways are shown in FIG. 1. Such a HHA, HHA-CoA, 6-HH, and/or HDO pathway as described herein can include one or more of: 1A) a thiolase (Thl) (alternatively referred to as “3-oxoadipyl-CoA thiolase” or “Beta-ketoadipyl-CoA thiolase”) that converts succinyl-CoA and acetyl-CoA into 3-oxoadipyl-CoA; (1B) a hydroxyadipyl-CoA dehydrogenase (Hbd) (alternatively referred to as “3-oxoadipyl-CoA dehydrogenase”) that converts 3-oxoadipyl-CoA to 3-hydroxyadipyl-CoA; 1C) a crotonase (Crt) (alternatively referred to as “3-oxoadipyl-CoA dehydratase”) that converts 3-hydroxyadipyl-CoA to 5-carboxy-2-pentenoyl-CoA; 1D) trans-enoyl CoA reductase (TER) (alternatively referred to as “5-carboxy-2-pentenoyl-CoA reductase” that converts 5-carboxy-2-pentenoyl-CoA to adipyl-CoA; 1E) a 6ACA-aldehyde dehydrogenase (6ACA-ALD) that converts adipyl CoA to adipate semialdehyde; 1F) an alcohol dehydrogenase which can convert adipate semialdehyde to 6-hydroxyhexanoate; an 1G) acyl-coenzyme A ligase (ACL), such as an engineered ACL as described herein, that catalyzes the conversion of HHA to HHA-CoA; 1H) acetylating aldehyde dehydrogenase (ALDH), such as an engineered ALDH as described herein, that catalyzes the conversion of HHA-CoA to 6-HH; 11) an alcohol dehydrogenase (ADH) that catalyzes the conversion of 6-HH to HDO; and, optionally, 1K) carboxylic acid reductase (CAR) which can catalyze the conversion of HHA to 6-HH.

An HHA pathway, as described herein as a precursor pathway for the downstream production of HHA-CoA, 6-HH, and/or HDO, can include: 1A; 1B; 1C; 1D; 1E; and 1F.

An HHA-CoA pathway that comprises an ACL as described herein can include: 1A; 1B; 1C; 1D; 1E; 1F; and 1G, such as an engineered ACL as described herein.

A 6-HH pathway that comprises an ACL as described herein and/or ALDH as described herein can include: 1A; 1B; 1C; 1D; 1E; 1F; 1G, such as an engineered ACL as described herein; and 1H such as an engineered ALDH as described herein.

A HDO pathway as described herein, that comprises an ACL and/or an ALDH as decreased herein can include: 1A; 1B; 1C; 1D; 1E; 1F; 1G, such as an engineered ACL as described herein; 1H such as an engineered ALDH as described herein; and 11.

Exemplary 6ACA, 6ACA-CoA, 6ACA-SADH, CPO and HMD pathways are shown in FIG. 2. Such a 6ACA, 6ACA-CoA, 6ACA-SADH, CPO and/or HMD pathway as described herein can include one or more of: 2A) 3-oxoadipyl-CoA thiolase that converts succinyl-CoA and acetyl-CoA to 3-oxoadipyl-CoA; 2B) 3-oxoadipyl-CoA reductransaminase that converts 3-oxoadipyl-CoA to 3-hydroxyadipyl-CoA; 2C) 3-hydroxyadipyl-CoA dehydratransaminase which converts that converts 3-hydroxyadipyl-CoA to 5-carboxy-2-pentenoyl-CoA; 2D) adipate semialdehydereductransaminase which converts 5-carboxy-2-pentenoyl-CoA to adipyl-CoA; 2E) 3-oxoadipyl-CoA/acyl-CoA transferase, 2F) 3-oxoadipyl-CoA synthase, or 2G) 3-oxoadipyl-CoA hydrolase which converts 3-oxoadipyl-CoA to 3-oxoadipate; 2H) 3-oxoadipate reductransaminase which converts 3-oxoadipate to 3-hydroxyadipate; 21) 3-hydroxyadipate dehydratransaminase which converts 3-hydroxyadipate to 5-carboxy-2-pentenoate; 2J) 5-carboxy-2-pentenoate reductransaminase which converts 5-carboxy-2-pentenoate to adipate; 2K) adipyl-CoA/acyl-CoA transferase, 2L) adipyl-CoA synthase or 2M) adipyl-CoA hydrolase which can reversibly convert adipyl-CoA and adipate; 2N) adipyl-CoA reductransaminase (aldehyde forming) which converts adipyl-CoA to adipate semialdehyde; 20) 6-aminocaproate transaminase or 2P) 6-aminocaproate dehydrogenase which converts adipate semialdehyde to 6ACA; 2Q) 6-aminocaproyl-CoA/acyl-CoA transferase or 2R) ACL which converts 6ACA to 6ACA-CoA; 2S) amidohydrolase which converts 6ACA to CPO; 2T) spontaneous cyclization of 6ACA-CoA to caprolactam; 2U) ALDH which converts 6ACA-CoA to 6ACA-SADH; 2V) HMD transaminase or 2W) HMD dehydrogenase which converts 6ACA-SADH to HMD; 2X) adipate reductase which converts adipate to adipate semialdehyde; 2Y) adipate kinase which converts adipate to adipylphosphate; and 2Z) adipylphosphate reductase which converts adipylphosphate to adipate semialdehyde.

An 6ACA pathway, as described herein as a precursor pathway for the downstream production of 6ACA-CoA, 6ACA-SADH, CPO and HMD, can include: 2A; 2B; 2C; 2D; 2N; and 2O or 2P. Another 6ACA pathway as described herein as a precursor pathway for the downstream production of 6ACA-CoA, 6ACA-SADH, CPO and HMD, can include: 2A; 2B; 2C; 2D; 2K, 2L, or 2M; 2X; and 2O or 2P. Another 6ACA pathway as described herein as a precursor pathway for the downstream production of 6ACA-CoA, 6ACA-SADH, CPO and HMD, can include: 2A; 2B; 2C; 2D; 2K, 2L, or 2M; 2Y; 2Z; and 2O or 2P. Another 6ACA pathway as described herein as a precursor pathway for the downstream production of 6ACA-CoA, 6ACA-SADH, CPO and HMD, can include: 2A; 2E, 2F, or 2G; 2H; 2I; 2J; 2K, 2L, or 2M; 2N; and 2O or 2P. Another 6ACA pathway as described herein as a precursor pathway for the downstream production of 6ACA-CoA, 6ACA-SADH, CPO and HMD, can include: 2A; 2E, 2F, or 2G; 2H; 2I; 2J; 2X; and 2O or 2P. Another 6ACA pathway as described herein as a precursor pathway for the downstream production of 6ACA-CoA, 6ACA-SADH, CPO and HMD, can include: 2A; 2E, 2F, or 2G; 2H; 2I; 2J; 2Y; 2Z; and 2O or 2P.

An 6ACA-CoA pathway that comprises an ACL as described herein, can include: 2A; 2B; 2C; 2D; 2N; 2O or 2P; and 2R, such as an engineered ACL as described herein. Another 6ACA-CoA pathway that comprises an ACL as described herein, can include: 2A; 2B; 2C; 2D; 2K, 2L, or 2M; 2X; 2O or 2P; and 2R, such as an engineered ACL as described herein. Another 6ACA-CoA pathway that comprises an ACL as described herein, can include: 2A; 2B; 2C; 2D; 2K, 2L, or 2M; 2Y; 2Z; 2O or 2P; and 2R, such as an engineered ACL as described herein. Another 6ACA-CoA pathway that comprises an ACL as described herein, can include: 2A; 2E, 2F, or 2G; 2H; 2I; 2J; 2K, 2L, or 2M; 2N; 2O or 2P; and 2R, such as an engineered ACL as described herein. Another 6ACA-CoA pathway that comprises an ACL as described herein, can include: 2A; 2E, 2F, or 2G; 2H; 2I; 2J; 2X; 2O or 2P; and 2R, such as an engineered ACL as described herein.

Another 6ACA-CoA pathway that comprises an ACL as described herein, can include: 2A; 2E, 2F, or 2G; 2H; 2I; 2J; 2Y; 2Z; 2O or 2P; and 2R, such as an engineered ACL as described herein.

A CPO pathway that comprises an ACL as described herein, can include: 2A; 2B; 2C; 2D; 2N; 2O or 2P; 2R, such as an engineered ACL as described herein; and a 2T. Another CPO pathway that comprises an ACL as described herein, can include: 2A; 2B; 2C; 2D; 2K, 2L, or 2M; 2X; 2O or 2P; 2R, such as an engineered ACL as described herein; and a 2T. Another CPO pathway that comprises an ACL as described herein, can include: 2A; 2B; 2C; 2D; 2K, 2L, or 2M; 2Y; 2Z; 2O or 2P; 2R, such as an engineered ACL as described herein; and a 2T. Another CPO pathway that comprises an ACL as described herein, can include: 2A; 2E, 2F, or 2G; 2H; 2I; 2J; 2K, 2L, or 2M; 2N; 2O or 2P; 2R, such as an engineered ACL as described herein; and a 2T. Another CPO pathway that comprises an ACL as described herein, can include: 2A; 2E, 2F, or 2G; 2H; 2I; 2J; 2X; 2O or 2P; 2R, such as an engineered ACL as described herein; and a 2T. Another CPO pathway that comprises an ACL as described herein, can include: 2A; 2E, 2F, or 2G; 2H; 2I; 2J; 2Y; 2Z; 2O or 2P; 2R, such as an engineered ACL as described herein; and a 2T.

An 6ACA-SADH pathway that comprises an ACL as described herein and/or an ALDH as described herein, can include: 2A; 2B; 2C; 2D; 2N; 2O or 2P; 2Q or 2R, such as an engineered ACL as described herein; and 2U, such as an engineered ALDH as described herein. Another 6ACA-SADH pathway that comprises an ACL as described herein and/or an ALDH as described herein, can include: 2A; 2B; 2C; 2D; 2K, 2L, or 2M; 2X; 2O or 2P; 2Q or 2R, such as an engineered ACL as described herein; and 2U, such as an engineered ALDH as described herein. Another 6ACA-SADH pathway that comprises an ACL as described herein and/or an ALDH as described herein, can include: 2A; 2B; 2C; 2D; 2K, 2L, or 2M; 2Y; 2Z; 2O or 2P; 2Q or 2R, such as an engineered ACL as described herein; and 2U, such as an engineered ALDH as described herein. Another 6ACA-SADH pathway that comprises an ACL as described herein and/or an ALDH as described herein, can include: 2A; 2E, 2F, or 2G; 2H; 2I; 2J; 2K, 2L, or 2M; 2N; 2O or 2P; 2Q or 2R, such as an engineered ACL as described herein; and 2U, such as an engineered ALDH as described herein. Another 6ACA-SADH pathway that comprises an ACL as described herein and/or an ALDH as described herein, can include: 2A; 2E, 2F, or 2G; 2H; 2I; 2J; 2X; 2O or 2P; 2Q or 2R, such as an engineered ACL as described herein; and 2U, such as an engineered ALDH as described herein. Another 6ACA-SADH pathway that comprises an ACL as described herein and/or an ALDH as described herein, can include: 2A; 2E, 2F, or 2G; 2H; 2I; 2J; 2Y; 2Z; 2O or 2P; 2Q or 2R, such as an engineered ACL as described herein; and 2U, such as an engineered ALDH as described herein.

An HMD pathway that comprises an ACL as described herein and/or an ALDH as described herein, can include: 2A; 2B; 2C; 2D; 2N; 2O or 2P; 2Q or 2R, such as an engineered ACL as described herein; 2U, such as an engineered ALDH as described herein; and 2V or 2W. Another HMD pathway that comprises an ACL as described herein and/or an ALDH as described herein, can include: 2A; 2B; 2C; 2D; 2K, 2L, or 2M; 2X; 2O or 2P; 2Q or 2R, such as an engineered ACL as described herein; 2U, such as an engineered ALDH as described herein; and 2V or 2W. Another HMD pathway that comprises an ACL as described herein and/or an ALDH as described herein, can include: 2A; 2B; 2C; 2D; 2K, 2L, or 2M; 2Y; 2Z; 2O or 2P; 2Q or 2R, such as an engineered ACL as described herein; 2U, such as an engineered ALDH as described herein; and 2V or 2W. Another HMD pathway that comprises an ACL as described herein and/or an ALDH as described herein, can include: 2A; 2E, 2F, or 2G; 2H; 2I; 2J; 2K, 2L, or 2M; 2N; 2O or 2P; 2Q or 2R, such as an engineered ACL as described herein; 2U, such as an engineered ALDH as described herein; and 2V or 2W. Another HMD pathway that comprises an ACL as described herein and/or an ALDH as described herein, can include: 2A; 2E, 2F, or 2G; 2H; 2I; 2J; 2X; 2O or 2P; 2Q or 2R, such as an engineered ACL as described herein; 2U, such as an engineered ALDH as described herein; and 2V or 2W. Another HMD pathway that comprises an ACL as described herein and/or an ALDH as described herein, can include: 2A; 2E, 2F, or 2G; 2H; 2I; 2J; 2Y; 2Z; 2O or 2P; 2Q or 2R, such as an engineered ACL as described herein; 2U, such as an engineered ALDH as described herein; and 2V or 2W.

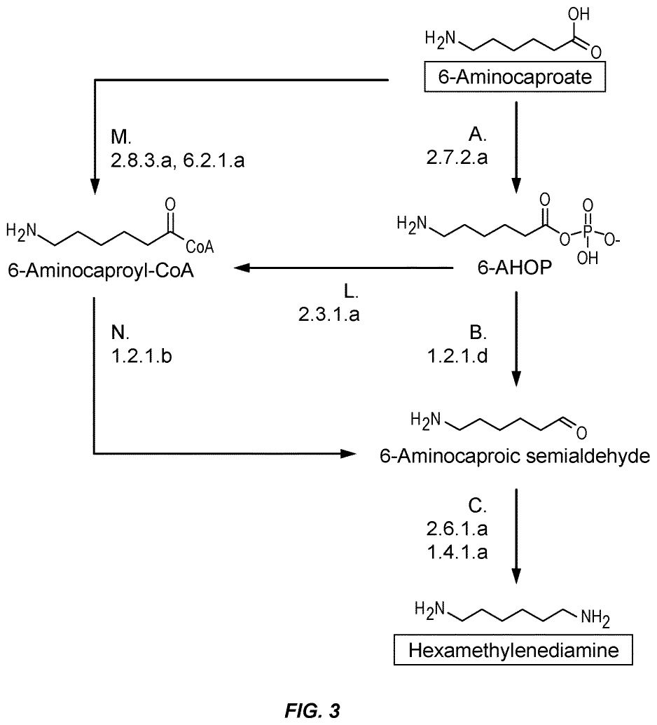

Exemplary 6ACA-SADH and HMD pathways are also shown in FIG. 3. Such a 6ACA-SADH, and/or HMD pathway as described herein can include can include one or more of: 3A) 6-aminocaproate kinase that converts 6-ACA to (6-aminohexanoyl)oxy]phosphonate (6-AHOP); 3B) 6-AHOP oxidoreductase that converts (6-aminohexanoyl)oxy]phosphonate to 6ACA-SADH; 3C) 6-aminocaproic semialdehyde aminotransferase and/or 6-aminocaproic semialdehyde oxidoreductase (aminating) which converts 6ACA-SADH to HMD; 3L) 6-AHOP acyltransferase which converts 6-AHOP to 6ACA-CoA; 3M) 6-aminocaproate CoA transferase and/or ACL, such as an engineered ACL as described herein, which converts 6ACA to 6ACA-CoA; and 3N) ALDH, such as an engineered ALDH as described herein which converts 6ACA-CoA to 6ACA-SADH.

An exemplary 6ACA-SADH pathway that comprises an ALDH as described herein, can include: 3A; 3L; and 3N, such as an engineered ALDH as described herein.

An exemplary HMD pathway that comprises an ALDH as described herein, can include: 3A; 3L; 3N, such as an engineered ALDH as described herein, and 3C.

Exemplary HHA, 6ACA-CoA, 6ACA-SADH, 6-aminohexanol, 6-HH, and/or HDO pathways are shown in FIG. 4. Such a 6ACA-CoA, 6ACA-SADH, 6-aminohexanol, 6-HH, and/or HDO pathway as described herein can include one or more of: 4A) 6-aminocaproyl-CoA transferase or synthetase, or an ACL, such as an ACL as described herein, which converts 6ACA to 6ACA-CoA; 4B) 6-aminocaproyl-CoA reductase, or an ALDH, such as an ALDH as described herein, which converts 6-aminocaproyl-CoA to 6-aminocaproate semialdehyde; 4C) 6-aminocaproate semialdehyde reductase which converts 6-aminocaproate semialdehyde to 6-aminohexanol; 4D) 6-aminocaproate reductase which converts 6ACA to 6-aminocaproate semialdehyde; 4E) adipyl-CoA reductase adipyl-CoA to adipate semialdehyde; 4F) adipate semialdehyde reductase which converts adipate semialdehyde to 6-hydroxyhexanoate; 4G) 6-hydroxyhexanoyl-CoA transferase or synthetase or an ACL, such as an ACL as described herein, which converts 6-hydroxyhexanoate to 6-hydroxyhexanoyl-CoA; 4H) 6-hydroxyhexanoyl-CoA reductase or an ALDH, such as an ALDH as described herein, which converts 6-hydroxyhexanoyl-CoA to 6-hydroxyhexanal; 41) 6-hydroxyhexanal reductase which converts 6-hydroxyhexanal to HDO; 4J) 6-aminohexanol aminotransferase or oxidoreductases which converts 6-aminohexanol to 6-hydroxyhexanal; 4K) 6-hydroxyhexanoate reductase which converts 6-hydroxyhexanoate to 6-hydroxyhexanal; 4L) adipate reductase which converts ADA to adipate semialdehyde; and 4M) adipyl-CoA transferase, hydrolase or synthase which converts adipyl-CoA to ADA.

Another HHA pathway, as described herein as a precursor pathway for the downstream production of HHA-CoA, 6-HH, and/or HDO, can include: 4M; 4E; and 4F. Another HHA pathway, as described herein as a precursor pathway for the downstream production of HHA-CoA, 6-HH, and/or HDO, can include: 4L and 4F.

Another HHA-CoA pathway, that comprises an ACL as described herein can include: 4M; 4E; 4F; and 4G, such as an ACL described herein. Another HHA-CoA pathway, that comprises an ACL as described herein can include: 4L 4F; and 4G, such as an ACL described herein.

Another 6-HH pathway, that comprises an ACL as described herein and/or an ALDH as described herein can include: 4M; 4E; 4F; 4G, such as an ACL described herein; and 4H such as an ALDH described herein. Another 6-HH pathway, that comprises an ACL as described herein and/or an ALDH as described herein can include: 4L 4F; 4G, such as an ACL described herein; and 4H such as an ALDH described herein. Another 6-HH pathway, that comprises an ACL as described herein and/or an ALDH as described herein can include: 4A, such as an ACL as described herein; 4B, such as an ALDH as described herein; 4C; 4J; and 41.

An 6ACA-CoA pathway that comprises an ACL as described herein, can include: 4A, such as an ACL as described herein.

A 6ACA-SADH pathway that comprises an ACL as described herein and/or an ALDH as described herein, can include: 4A, such as an ACL as described herein; and 4B, such as an ALDH as described herein.

A 6ACA-aminohexanol pathway that comprises an ACL as described herein and/or an ALDH as described herein, can include: 4A, such as an ACL as described herein; 4B, such as an ALDH as described herein; and 4C.

Another HDO pathway, that comprises an ACL as described herein and/or an ALDH as described herein can include: 4M; 4E; 4F; 4G, such as an ACL described herein; 4H such as an ALDH described herein; and 41. Another HDO pathway, that comprises an ACL as described herein and/or an ALDH as described herein can include: 4L 4F; 4G, such as an ACL described herein; and 4H such as an ALDH described herein. Another HDO pathway, that comprises an ACL as described herein and/or an ALDH as described herein can include: 4A, such as an ACL as described herein; 4B, such as an ALDH as described herein; 4C; 4J; and 41.

Either ACL or ALDH enzymes described herein, or both, can be used in any of the biosynthetic pathways described herein so long as the substrate and products of the referenced ACL and/or ALDH enzymatic conversion or conversions are intermediates within the described pathway. For example, as disclosed herein, an engineered ACL described herein can function in a pathway to convert HHA to HHA-CoA. In the pathway described above that includes an ACL that converts HHA to HHA-CoA, the pathway converts HHA to HHA-CoA. An engineered ACL described herein can also be used in other HHA-CoA and/or HDO pathways that comprise HHA as a substrate/product in the pathway. One skilled in the art can readily utilize an engineered ACL described herein to convert HHA to HHA-CoA in any desired pathway that comprises such a reaction. In another example, as disclosed herein, an engineered ACL described herein can function in a pathway to convert 6ACA to 6ACA-CoA. In the pathway described above that includes an ACL that converts 6ACA to 6ACA-CoA, the pathway converts 6ACA to 6ACA-CoA. An engineered ACL described herein can also be used in other 6ACA-CoA, HMD, and/or HDO pathways that comprise 6ACA as a substrate/product in the pathway. One skilled in the art can readily utilize an engineered ACL described herein to convert 6ACA to 6ACA-CoA in any desired pathway that comprises such a reaction.

As disclosed herein, an engineered ALDH described herein can function in a pathway to convert HHA-CoA to 6-HH. In the pathway described above that includes an ALDH that converts HHA-CoA to 6-HH, the pathway converts HHA-CoA to 6-HH. An engineered ALDH described herein can also be used in other 6-HH and/or HDO pathways that comprise HHA-CoA as a substrate/product in the pathway. One skilled in the art can readily utilize an engineered ALDH described herein to convert HHA-CoA to 6-HH in any desired pathway that comprises such a reaction. In another example, an engineered ALDH described herein can function in a pathway to convert 6ACA-CoA to 6ACA-SADH. In the pathway described above that includes an ALDH that converts 6ACA-CoA to 6ACA-SADH, the pathway converts 6ACA-CoA to 6ACA-SADH. An engineered ALDH described herein can also be used in other 6ACA-SADH, HMD, and/or HDO pathways that comprise 6ACA-CoA as a substrate/product in the pathway. One skilled in the art can readily utilize an engineered ALDH described herein to convert 6ACA-CoA to 6ACA-SADH in any desired pathway that comprises such a reaction.

Enzyme types required to convert common central metabolic intermediates into HDO, HMD or intermediates are indicated above with representative Enzyme Commission (EC) numbers (see also WO 2010/127319, WO 2013/036764, WO 2008/115840, WO 2010/030711, WO 2010/141920, WO 2011/047101, WO 2013/184602, WO 2014/176514, U.S. Pat. Nos. 9,017,983, 8,067,214, 7,858,350, 8,129,169, 8,377,666, US 2013/0066035, US 2013/0029381, US 2014/0030779, US 2015/0148513, US 2014/0371417, US 2019/0300919, US 2007/0254341, US 2011/0091944, US 2011/0171699, WO 2009/151728, WO 2010/129936, and WO 2021/216952). The first three digits of each label correspond to the first three Enzyme Commission number digits which denote the general type of transformation independent of substrate specificity. Exemplary enzymes include: 1.1.1.a, Oxidoreductase (ketone to hydroxyl or aldehyde to alcohol); 1.1.1.c, Oxidoreductase (2 step, acyl-CoA to alcohol); 1.2.1.b, Oxidoreductase (acyl-CoA to aldehyde); 1.2.1.c, Oxidoreductase (2-oxo acid to acyl-CoA, decarboxylation); 1.2.1.d, Oxidoreductase (phosphorylating/dephosphorylating); 1.3.1.a, Oxidoreductase operating on CH—CH donors; 1.4.1.a, Oxidoreductase operating on amino acids (deaminating); 2.3.1.a, Acyltransferase (transferring phosphate group); 2.6.1.a, Aminotransferase; 2.7.2.a, Phosphotransferase, carboxyl group acceptor; 2.8.3.a, Coenzyme-A transferase; 3.1.2.a, Thiolester hydrolase (CoA specific); 3.5.1.a, hydrolase; 3.5.2.-, in cyclic amides; 4.1.1.a, Carboxy-lyase; 4.2.1.a, Hydro-lyase; 4.3.1.a, Ammonia-lyase; 5.3.3.a, Isomerase; 5.4.3.a, Aminomutase; and 6.2.1.a, Acid-thiol ligase.

The bio-derived form of HHA-CoA, 6-HH, HDO, 6ACA-CoA, 6ACA-SADH, CPO, and/or HMD, can be utilized for production of downstream products in which a non-petroleum based product is desired, for example, by substituting petroleum-derived HDO with the bio-derived form of HDO as described herein. Accordingly, in some embodiments, provided herein is a bioderived HDO, or downstream products related thereto, such as an ester or amide thereof, or the biosynthesis of nylon and intermediates thereof.

An engineered ACL described herein can be utilized in a cell or in vitro to convert an acid to its corresponding acyl-CoA. As disclosed herein, the engineered ACLs described herein have beneficial and useful properties, including but not limited to increased specificity for HHA or 6ACA, or both over: acetate, adipate, propionate, succinate, 3HB, and/or 4HB; increased activity; decreased by-product production, and the like.

An engineered ALDH described herein can be utilized in a cell or in vitro to convert an acyl-CoA to its corresponding aldehyde. As disclosed herein, the engineered aldehyde dehydrogenases described herein have beneficial and useful properties, including but not limited to increased specificity HHA-CoA, 6ACA-CoA, or both over: adipyl-CoA, acetyl-CoA, succinyl-CoA, and/or 3-hydroxyadipyl-CoA; increased activity; decreased by-product production, and the like.

While generally described herein as a cell that contains a HHA-CoA, 6-HH, HDO, 6ACA-CoA, 6ACA-SADH, CPO, or HMD pathway comprising an engineered aldehyde dehydrogenase provided herein, it is understood that provided herein is a cell comprising at least one recombinant nucleic acid encoding an engineered aldehyde dehydrogenase provided herein. The aldehyde dehydrogenase can be expressed in a sufficient amount to produce a desired product, such a product of a HHA-CoA, 6-HH, HDO, 6ACA-CoA, 6ACA-SADH, CPO, or HMD pathway, or a downstream product related thereto such as an ester or amide thereof. Exemplary HHA-CoA, 6-HH, HDO, 6ACA-CoA, 6ACA-SADH, CPO, or HMD pathways are described herein.

It is understood that any of the pathways disclosed herein, as described in the Examples, including the pathways described herein, can be utilized to generate a cell that produces any pathway intermediate or product, as desired, in particular a pathway that utilizes an engineered ACL and/or an engineered ALDH as provided herein. As disclosed herein, such a cell that produces an intermediate can be used in combination with another cell expressing one or more upstream or downstream pathway enzymes to produce a desired product. However, it is understood that a cell that produces a HHA-CoA, 6-HH, HDO, 6ACA-CoA, 6ACA-SADH, CPO, or HMD pathway intermediate can be utilized to produce the intermediate as a desired product.

The subject matter described herein includes general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well-known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

The cells provided herein can be produced by introducing an expressible nucleic acid encoding an engineered ACL and/or engineered ALDH provided herein, and optionally expressible nucleic acids encoding one or more of the enzymes or proteins participating in one or more HHA-CoA, 6-HH, HDO, 6ACA-CoA, 6ACA-SADH, CPO, or HMD biosynthetic pathways, and further optionally a nucleic acid encoding an enzyme that produces a downstream product related to HHA-CoA, 6-HH, HDO, 6ACA-CoA, 6ACA-SADH, CPO, or HMD such as an ester or amide thereof. Depending on the host cell chosen, nucleic acids for some or all of a particular HHA-CoA, 6-HH, HDO, 6ACA-CoA, 6ACA-SADH, CPO, or HMD biosynthetic pathway, or downstream product, can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is included for the deficient enzyme(s) or protein(s) to achieve HHA-CoA, 6-HH, HDO, 6ACA-CoA, 6ACA-SADH, CPO, or HMD biosynthesis, or exogenous expression of endogenously expressed genes can be provided to increase expression of pathway enzymes, if desired. Thus, a cell provided herein can be produced by introducing an engineered ACL and/or engineered ALDH provided herein, and optionally exogenous enzyme or protein activities to obtain a desired biosynthetic pathway, or by introducing one or more exogenous enzyme or protein activities, including an engineered ACL and/or engineered ALDH provided herein that, together with one or more endogenous enzymes or proteins, produces a desired product such as HHA-CoA, 6-HH, HDO, 6ACA-CoA, 6ACA-SADH, CPO, or HMD, or a downstream product related thereto such as an ester or amide thereof.

### Host Cells

Host cells can be selected from, and the non-naturally cells expressing an engineered ACL and/or engineered ALDH provided herein generated in, for example, bacteria, yeast, fungus or any of a variety of microorganisms applicable or suitable to fermentation processes. In some embodiments, the microbial organism is a species of bacteria, yeast or fungus. In some embodiments, the microbial organism is a species of bacteria. In some embodiments, the microbial organism is a species of yeast. In some embodiments, the microbial organism is a species of fungus. Exemplary bacteria include any species selected from the order Enterobacteriales, family Enterobacteriaceae, including the genera Escherichia and Klebsiella; the order Aeromonadales, family Succinivibrionaceae, including the genus Anaerobiospirillum; the order Pasteurellales, family Pasteurellaceae, including the genera Actinobacillus and Mannheimia; the order Rhizobiales, family Bradyrhizobiaceae, including the genus Rhizobium; the order Bacillales, family Bacillaceae, including the genus Bacillus; the order Actinomycetales, families Corynebacteriaceae and Streptomycetaceae, including the genus Corynebacterium and the genus Streptomyces, respectively; order Rhodospirillales, family Acetobacteraceae, including the genus Gluconobacter; the order Sphingomonadales, family Sphingomonadaceae, including the genus Zymomonas; the order Lactobacillales, families Lactobacillaceae and Streptococcaceae, including the genus Lactobacillus and the genus Lactococcus, respectively; the order Clostridiales, family Clostridiaceae, genus Clostridium; and the order Pseudomonadales, family Pseudomonadaceae, including the genus Pseudomonas. Non-limiting species of host bacteria include Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. E. coli is a particularly useful host organism since it is a well characterized microbial organism suitable for genetic engineering.

Similarly, exemplary species of yeast or fungi species include any species selected from the order Saccharomycetales, family Saccaromycetaceae, including the genera Saccharomyces, Kluyveromyces and Pichia; the order Saccharomycetales, family Dipodascaceae, including the genus Yarrowia; the order Schizosaccharomycetales, family Schizosaccaromycetaceae, including the genus Schizosaccharomyces; the order Eurotiales, family Trichocomaceae, including the genus Aspergillus; and the order Mucorales, family Mucoraceae, including the genus Rhizopus. Non-limiting species of host yeast or fungi include Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, and the like. A particularly useful host organism that is a yeast includes Saccharomyces cerevisiae.

Although generally described herein as utilizing a cell that is a microbial organism as a host cell, particularly for producing HHA-CoA, 6-HH, HDO, 6ACA-CoA, 6ACA-SADH, CPO, and/or HMD, or a downstream product related thereto such as an ester or amide thereof, it is understood that a host cell can be a cell line of a higher eukaryote, such as a mammalian cell line or insect cell line. Thus, it is understood that reference herein to a host cell that is a microbial organism can alternatively utilize a higher eukaryotic cell line to produce a desired product. Exemplary higher eukaryotic cell lines include, but are not limited to, Chinese hamster ovary (CHO), human (Hela, Human Embryonic Kidney (HEK) 293, Jurkat), mouse (3T3), primate (Vero), insect (Sf9), and the like. Such cell lines are commercially available (see, for example, the American Type Culture Collection (ATCC; Manassas VA); Life Technologies, Carlsbad CA). It is understood that any suitable host cell can be used to introduce an engineered ACL and/or engineered ALDH provided herein, and optionally metabolic and/or genetic modifications to produce a desired product.

Depending on the HHA-CoA, 6-HH, HDO, 6ACA-CoA, 6ACA-SADH, CPO, or HMD, biosynthetic pathway constituents of a selected host cell, the non-naturally occurring cells provided herein will include at least one exogenously expressed HHA-CoA, 6-HH, HDO, 6ACA-CoA, 6ACA-SADH, CPO, or HMD pathway-encoding nucleic acid and up to all encoding nucleic acids for one or more HHA-CoA, 6-HH, HDO, 6ACA-CoA, 6ACA-SADH, CPO, or HMD biosynthetic pathways, or a downstream product related thereto such as an ester or amide thereof, including an engineered ACL and/or engineered ALDH provided herein. For example, HHA-CoA, 6-HH, HDO, 6ACA-CoA, 6ACA-SADH, CPO, or HMD biosynthesis can be established in a host deficient in a pathway enzyme or protein through exogenous expression of the corresponding encoding nucleic acid, including an engineered ACL and/or engineered ALDH provided herein. In a host deficient in all enzymes or proteins of a HHA-CoA, 6-HH, HDO, 6ACA-CoA, 6ACA-SADH, CPO, or HMD pathway, or a downstream product related thereto such as an ester or amide thereof, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of HHA-CoA, 6-HH, HDO, 6ACA-CoA, 6ACA-SADH, CPO, or HMD pathway, or a downstream product related thereto such as an ester or amide thereof, can be included, including an engineered ACL and/or engineered ALDH provided herein.

Given the teachings and guidance provided herein, those skilled in the art will understand that the number of encoding nucleic acids to introduce in an expressible form will, at least, parallel the HHA-CoA, 6-HH, HDO, 6ACA-CoA, 6ACA-SADH, CPO, or HMD pathway deficiencies of the selected host cell if a HHA-CoA, 6-HH, HDO, 6ACA-CoA, 6ACA-SADH, CPO, or HMD pathway is to be included in the cell. Therefore, a non-naturally occurring cell provided herein can have one, two, three, four, five, six, seven, eight, and so forth, depending on the particular pathway, up to all nucleic acids encoding the enzymes or proteins constituting a HHA-CoA, 6-HH, HDO, 6ACA-CoA, 6ACA-SADH, CPO, or HMD biosynthetic pathway disclosed herein. In some embodiments, the non-naturally occurring cells also can include other genetic modifications that facilitate or optimize HHA-CoA, 6-HH, HDO, 6ACA-CoA, 6ACA-SADH, CPO, or HMD biosynthesis or that confer other useful functions onto the host cell. One such other functionality can include, for example, augmentation of the synthesis of one or more of the HHA-CoA, 6-HH, HDO, 6ACA-CoA, 6ACA-SADH, CPO, or HMD pathway precursors such acetyl-CoA or succinyl-CoA.

Generally, a host cell is selected such that it can express an engineered ACL and/or engineered ALDH provided herein, and optionally produces the precursor of a HHA-CoA, 6-HH, HDO, 6ACA-CoA, 6ACA-SADH, CPO, or HMD pathway, in a cell containing such a pathway, either as a naturally produced molecule or as an engineered product that either provides de novo production of a desired precursor or increased production of a precursor naturally produced by the host cell. A host organism can be engineered to increase production of a precursor, as disclosed herein. In addition, a cell that has been engineered to produce a desired precursor can be used as a host organism and further engineered to express enzymes or proteins of a HHA-CoA, 6-HH, HDO, 6ACA-CoA, 6ACA-SADH, CPO, or HMD pathway, or a downstream product related thereto such as an ester or amide thereof, if desired.

In some embodiments, a non-naturally occurring cell provided herein is generated from a host that contains the enzymatic capability to synthesize HHA-CoA, 6-HH, HDO, 6ACA-CoA, 6ACA-SADH, CPO, or HMD, or a downstream product related thereto such as an ester or amide thereof. In this specific embodiment it can be useful to increase the synthesis or accumulation of a HHA-CoA, 6-HH, HDO, 6ACA-CoA, 6ACA-SADH, CPO, or HMD pathway product to, for example, drive HHA-CoA, 6-HH, HDO, 6ACA-CoA, 6ACA-SADH, CPO, or HMD pathway reactions toward HHA-CoA, 6-HH, HDO, 6ACA-CoA, 6ACA-SADH, CPO, or HMD production, or a downstream product related thereto such as an ester or amide thereof. Increased synthesis or accumulation can be accomplished by, for example, overexpression of nucleic acids encoding one or more of the above-described HHA-CoA, 6-HH, HDO, 6ACA-CoA, 6ACA-SADH, CPO, or HMD pathway enzymes or proteins, including an engineered ACL and/or engineered ALDH provided herein. Overexpression of the enzyme or enzymes and/or protein or proteins of the HHA-CoA, 6-HH, HDO, 6ACA-CoA, 6ACA-SADH, CPO, or HMD pathway can occur, for example, through exogenous expression of the endogenous gene or genes, or through exogenous expression of the heterologous gene or genes, including exogenous expression of an engineered ACL and/or engineered ALDH provided herein. Therefore, naturally occurring organisms can be readily converted to non-naturally occurring cells provided herein, for example, producing HHA-CoA, 6-HH, HDO, 6ACA-CoA, 6ACA-SADH, CPO, or HMD or a downstream product related thereto such as an ester or amide thereof, through overexpression of one, two, three, four, five, six, seven, eight, or more, depending on the HHA-CoA, 6-HH, HDO, 6ACA-CoA, 6ACA-SADH, CPO, or HMD pathway, that is, up to all nucleic acids encoding HHA-CoA, 6-HH, HDO, 6ACA-CoA, 6ACA-SADH, CPO, or HMD biosynthetic pathway enzymes or proteins, or enzymes that produce a downstream product related thereto such as an ester or amide thereof. In addition, a non-naturally occurring organism can be generated by mutagenesis of an endogenous gene that results in an increase in activity of an enzyme in the HHA-CoA, 6-HH, HDO, 6ACA-CoA, 6ACA-SADH, CPO, or HMD biosynthetic pathway, or a downstream product related thereto such as an ester or amide thereof.

### Variant Activity

In some embodiments, provided herein is a non-naturally occurring microbial organism that is a capable of producing more HHA-CoA, 6-HH, HDO, 6ACA-CoA, 6ACA-SADH, CPO, and/or HMD, or an ester or amide thereof compared to a control microbial organism that does not have a recombinant nucleic acid that encodes an engineered ACL and/or engineered ALDH described herein. Such a microbial organism, in some embodiments, is capable of producing at least 10% more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 20% more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 30% more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 40% more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 50% more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 60% more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 70% more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 80% more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 90% more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 1 fold more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 1.1 fold more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 1.2 fold more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 1.3 fold more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 1.4 fold more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 1.5 fold more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 1.6 fold more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 1.7 fold more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 1.8 fold more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 1.9 fold more HHA-CoA, 6-HH, HDO, 6ACA-CoA, 6ACA-SADH, CPO, and/or HMD, or an ester or amide thereof compared to the control microbial organism. In some embodiments, the microbial organism is capable of producing at least 2 fold more HHA-CoA, 6-HH, HDO, 6ACA-CoA, 6ACA-SADH, CPO, and/or HMD, or an ester or amide thereof compared to the control microbial organism.

In some embodiments, provided herein is a non-naturally occurring microbial organism that is a capable of producing more HHA-CoA, 6ACA-CoA, HDO, and/or HMD, or an ester or amide thereof compared to a control microbial organism that does not have a recombinant nucleic acid that encodes an engineered ACL described herein. Such a microbial organism, in some embodiments, is capable of producing at least 10% more HHA-CoA, 6ACA-CoA, HDO, and/or HMD, or an ester or amide thereof compared to the control microbial organism. In some embodiments, the microbial organism is capable of producing at least 20% more HHA-CoA, 6ACA-CoA, HDO, and/or HMD, or an ester or amide thereof compared to the control microbial organism. In some embodiments, the microbial organism is capable of producing at least 30% more HHA-CoA, 6ACA-CoA, HDO, and/or HMD, or an ester or amide thereof compared to the control microbial organism. In some embodiments, the microbial organism is capable of producing at least 40% more HHA-CoA, 6ACA-CoA, HDO, and/or HMD, or an ester or amide thereof compared to the control microbial organism. In some embodiments, the microbial organism is capable of producing at least 50% more HHA-CoA, 6ACA-CoA, HDO, and/or HMD, or an ester or amide thereof compared to the control microbial organism. In some embodiments, the microbial organism is capable of producing at least 60% more HHA-CoA, 6ACA-CoA, HDO, and/or HMD, or an ester or amide thereof compared to the control microbial organism. In some embodiments, the microbial organism is capable of producing at least 70% more HHA-CoA, 6ACA-CoA, HDO, and/or HMD, or an ester or amide thereof compared to the control microbial organism. In some embodiments, the microbial organism is capable of producing at least 80% more HHA-CoA, 6ACA-CoA, HDO, and/or HMD, or an ester or amide thereof compared to the control microbial organism. In some embodiments, the microbial organism is capable of producing at least 90% more HHA-CoA, 6ACA-CoA, HDO, and/or HMD, or an ester or amide thereof compared to the control microbial organism. In some embodiments, the microbial organism is capable of producing at least 1 fold more HHA-CoA, 6ACA-CoA, HDO, and/or HMD, or an ester or amide thereof compared to the control microbial organism. In some embodiments, the microbial organism is capable of producing at least 1.1 fold more HHA-CoA, 6ACA-CoA, HDO, and/or HMD, or an ester or amide thereof compared to the control microbial organism. In some embodiments, the microbial organism is capable of producing at least 1.2 fold more HHA-CoA, 6ACA-CoA, HDO, and/or HMD, or an ester or amide thereof compared to the control microbial organism. In some embodiments, the microbial organism is capable of producing at least 1.3 fold more HHA-CoA, 6ACA-CoA, HDO, and/or HMD, or an ester or amide thereof compared to the control microbial organism. In some embodiments, the microbial organism is capable of producing at least 1.4 fold more HHA-CoA, 6ACA-CoA, HDO, and/or HMD, or an ester or amide thereof compared to the control microbial organism. In some embodiments, the microbial organism is capable of producing at least 1.5 fold more HHA-CoA, 6ACA-CoA, HDO, and/or HMD, or an ester or amide thereof compared to the control microbial organism. In some embodiments, the microbial organism is capable of producing at least 1.6 fold more HHA-CoA, 6ACA-CoA, HDO, and/or HMD, or an ester or amide thereof compared to the control microbial organism. In some embodiments, the microbial organism is capable of producing at least 1.7 fold more HHA-CoA, 6ACA-CoA, HDO, and/or HMD, or an ester or amide thereof compared to the control microbial organism. In some embodiments, the microbial organism is capable of producing at least 1.8 fold more HHA-CoA, 6ACA-CoA, HDO, and/or HMD, or an ester or amide thereof compared to the control microbial organism. In some embodiments, the microbial organism is capable of producing at least 1.9 fold more HHA-CoA, 6ACA-CoA, HDO, and/or HMD, or an ester or amide thereof compared to the control microbial organism. In some embodiments, the microbial organism is capable of producing at least 2 fold more HHA-CoA, 6ACA-CoA, HDO, and/or HMD, or an ester or amide thereof compared to the control microbial organism.

In some embodiments, provided herein is a non-naturally occurring microbial organism that is a capable of producing more 6-HH, 6ACA-SADH, HDO, and/or HMD, or an ester or amide thereof compared to a control microbial organism that does not have a recombinant nucleic acid that encodes an engineered ALDH described herein. Such a microbial organism, in some embodiments, is capable of producing at least 10% more 6-HH, 6ACA-SADH, HDO, and/or HMD, or an ester or amide thereof compared to the control microbial organism. In some embodiments, the microbial organism is capable of producing at least 20% more 6-HH, 6ACA-SADH, HDO, and/or HMD, or an ester or amide thereof compared to the control microbial organism. In some embodiments, the microbial organism is capable of producing at least 30% more 6-HH, 6ACA-SADH, HDO, and/or HMD, or an ester or amide thereof compared to the control microbial organism. In some embodiments, the microbial organism is capable of producing at least 40% more 6-HH, 6ACA-SADH, HDO, and/or HMD, or an ester or amide thereof compared to the control microbial organism. In some embodiments, the microbial organism is capable of producing at least 50% more 6-HH, 6ACA-SADH, HDO, and/or HMD, or an ester or amide thereof compared to the control microbial organism. In some embodiments, the microbial organism is capable of producing at least 60% more 6-HH, 6ACA-SADH, HDO, and/or HMD, or an ester or amide thereof compared to the control microbial organism. In some embodiments, the microbial organism is capable of producing at least 70% more 6-HH, 6ACA-SADH, HDO, and/or HMD, or an ester or amide thereof compared to the control microbial organism. In some embodiments, the microbial organism is capable of producing at least 80% more 6-HH, 6ACA-SADH, HDO, and/or HMD, or an ester or amide thereof compared to the control microbial organism. In some embodiments, the microbial organism is capable of producing at least 90% more 6-HH, 6ACA-SADH, HDO, and/or HMD, or an ester or amide thereof compared to the control microbial organism. In some embodiments, the microbial organism is capable of producing at least 1 fold more 6-HH, 6ACA-SADH, HDO, and/or HMD, or an ester or amide thereof compared to the control microbial organism. In some embodiments, the microbial organism is capable of producing at least 1.1 fold more 6-HH, 6ACA-SADH, HDO, and/or HMD, or an ester or amide thereof compared to the control microbial organism. In some embodiments, the microbial organism is capable of producing at least 1.2 fold more 6-HH, 6ACA-SADH, HDO, and/or HMD, or an ester or amide thereof compared to the control microbial organism. In some embodiments, the microbial organism is capable of producing at least 1.3 fold more 6-HH, 6ACA-SADH, HDO, and/or HMD, or an ester or amide thereof compared to the control microbial organism. In some embodiments, the microbial organism is capable of producing at least 1.4 fold more 6-HH, 6ACA-SADH, HDO, and/or HMD, or an ester or amide thereof compared to the control microbial organism. In some embodiments, the microbial organism is capable of producing at least 1.5 fold more 6-HH, 6ACA-SADH, HDO, and/or HMD, or an ester or amide thereof compared to the control microbial organism. In some embodiments, the microbial organism is capable of producing at least 1.6 fold more 6-HH, 6ACA-SADH, HDO, and/or HMD, or an ester or amide thereof compared to the control microbial organism. In some embodiments, the microbial organism is capable of producing at least 1.7 fold more 6-HH, 6ACA-SADH, HDO, and/or HMD, or an ester or amide thereof compared to the control microbial organism. In some embodiments, the microbial organism is capable of producing at least 1.8 fold more 6-HH, 6ACA-SADH, HDO, and/or HMD, or an ester or amide thereof compared to the control microbial organism. In some embodiments, the microbial organism is capable of producing at least 1.9 fold more 6-HH, 6ACA-SADH, HDO, and/or HMD, or an ester or amide thereof compared to the control microbial organism. In some embodiments, the microbial organism is capable of producing at least 2 fold more 6-HH, 6ACA-SADH, HDO, and/or HMD, or an ester or amide thereof compared to the control microbial organism.

In some embodiments, provided herein is a non-naturally occurring microbial organism that produces a decreased amount of a by-product as compared to a control microbial organism that does not have a recombinant nucleic acid that encodes an engineered ACL and/or engineered ALDH described herein. By-products that can be decreased by using an engineered ACL and/or engineered ALDH provided herein in a non-naturally occurring microbial organism include any product that is not the desired product the engineered ACL and/or engineered ALDH is intended to catalyze the production of or a product that represents poor ACL and/or engineered ALDH activity. Non-limiting examples of such a by-product include adipic acid (ADA), 4-HB, acetate, succinate, 3-oxoadipate, 3-hydroxyadipate, 2-hexenedioate or combinations thereof. Accordingly, in some embodiments, the microbial organism provided herein is capable of producing at least 10% less by-product. In some embodiments, the microbial organism provided herein is capable of producing at least 20% less by-product. In some embodiments, the microbial organism provided herein is capable of producing at least 30% less by-product. In some embodiments, the microbial organism provided herein is capable of producing at least 40% less by-product. In some embodiments, the microbial organism provided herein is capable of producing at least 50% less by-product. In some embodiments, the microbial organism provided herein is capable of producing at least 60% less by-product. In some embodiments, the microbial organism provided herein is capable of producing at least 70% less by-product. In some embodiments, the microbial organism provided herein is capable of producing at least 80% less by-product. In some embodiments, the microbial organism provided herein is capable of producing at least 90% less by-product. In some embodiments, the microbial organism provided herein is capable of producing at least 95% less by-product. In some embodiments, the microbial organism provided herein is capable of producing at least 98% less by-product. In some embodiments, the microbial organism provided herein is capable of producing at least 99% less by-product.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring cell.

It is understood that, in methods described herein, any of the one or more recombinant and/or exogenous nucleic acids can be introduced into a cell to produce a non-naturally occurring cell provided herein. The nucleic acids can be introduced so as to confer, for example, a HHA-CoA, 6-HH, HDO, 6ACA-CoA, 6ACA-SADH, CPO, or HMD, or a downstream product related thereto such as an ester or amide thereof, biosynthetic pathway onto the cell, including introducing a nucleic acid encoding an engineered ACL and/or engineered ALDH provided herein. Alternatively, encoding nucleic acids can be introduced to produce a cell having the biosynthetic capability to catalyze some of the required reactions to confer HHA-CoA, 6-HH, HDO, 6ACA-CoA, 6ACA-SADH, CPO, or HMD biosynthetic capability to produce an intermediate. For example, a non-naturally occurring cell having a HHA-CoA, 6-HH, HDO, 6ACA-CoA, 6ACA-SADH, CPO, or HMD biosynthetic pathway can comprise at least two exogenous nucleic acids encoding desired enzymes or proteins, including an engineered ACL and/or engineered ALDH provided herein. Thus, it is understood that any combination of two or more enzymes or proteins of a biosynthetic pathway can be included in a non-naturally occurring cell provided herein, including an engineered ACL and/or engineered ALDH provided herein. Similarly, it is understood that any combination of three or more enzymes or proteins of a biosynthetic pathway can be included in a non-naturally occurring cell provided herein, as desired, so long as the combination of enzymes and/or proteins of the desired biosynthetic pathway results in production of the corresponding desired product. Similarly, any combination of five, six, seven, eight, nine, ten, eleven, twelve or more enzymes or proteins of a biosynthetic pathway as disclosed herein can be included in a non-naturally occurring cell provided herein, as desired, so long as the combination of enzymes and/or proteins of the desired biosynthetic pathway results in production of the corresponding desired product.

In addition to the biosynthesis of HHA-CoA, 6-HH, HDO, 6ACA-CoA, 6ACA-SADH, CPO, or HMD, or a downstream product related thereto such as an ester or amide thereof, as described herein, the non-naturally occurring cells and methods provided herein also can be utilized in various combinations with each other and/or with other cells and methods well known in the art to achieve product biosynthesis by other routes. For example, one alternative to produce HHA-CoA, 6-HH, HDO, 6ACA-CoA, 6ACA-SADH, CPO, or HMD other than use of the HHA-CoA, 6-HH, HDO, 6ACA-CoA, 6ACA-SADH, CPO, or HMD producers is through addition of another cell capable of converting a HHA-CoA, 6-HH, HDO, 6ACA-CoA, 6ACA-SADH, CPO, or HMD pathway intermediate to HHA-CoA, 6-HH, HDO, 6ACA-CoA, 6ACA-SADH, CPO, or HMD. One such procedure can include the fermentation of a cell that produces a HHA-CoA, 6-HH, HDO, 6ACA-CoA, 6ACA-SADH, CPO, or HMD pathway intermediate. The HHA-CoA, 6-HH, HDO, 6ACA-CoA, 6ACA-SADH, CPO, or HMD pathway intermediate can then be used as a substrate for a second cell that converts the HHA-CoA, 6-HH, HDO, 6ACA-CoA, 6ACA-SADH, CPO, or HMD pathway intermediate to HHA-CoA, 6-HH, HDO, 6ACA-CoA, 6ACA-SADH, CPO, or HMD. The HHA-CoA, 6-HH, HDO, 6ACA-CoA, 6ACA-SADH, CPO, or HMD pathway intermediate can be added directly to another culture of the second organism or the original culture of the HHA-CoA, 6-HH, HDO, 6ACA-CoA, 6ACA-SADH, CPO, or HMD pathway intermediate producers can be depleted of these cells by, for example, cell separation, and then subsequent addition of the second organism to the fermentation broth can be utilized to produce the final product without intermediate purification steps. A cell that produces a downstream product related to HHA-CoA, 6-HH, HDO, 6ACA-CoA, 6ACA-SADH, CPO, or HMD such as an ester or amide thereof, can optionally be included to produce such a downstream product.

Alternatively, such enzymatic conversions can be carried out in vitro, with a combination of enzymes or sequential exposure of substrates to enzymes that result in conversion of a substrate to a desired product. As another alternative, a combination of cell-based conversions and in vitro enzymatic conversions can be used, if desired.

In other embodiments, the non-naturally occurring cells and methods provided herein can be assembled in a wide variety of subpathways to achieve biosynthesis of, for example, HHA-CoA, 6-HH, HDO, 6ACA-CoA, 6ACA-SADH, CPO, or HMD or a downstream product related thereto such as an ester or amide thereof. In these embodiments, biosynthetic pathways for a desired product provided herein can be segregated into different cells, and the different cells can be co-cultured to produce the final product. In such a biosynthetic scheme, the product of one cell is the substrate for a second cell until the final product is synthesized. For example, the biosynthesis of HHA-CoA, 6-HH, HDO, 6ACA-CoA, 6ACA-SADH, CPO, or HMD, or a downstream product related thereto such as an ester or amide thereof, can be accomplished by constructing a cell that contains biosynthetic pathways for conversion of one pathway intermediate to another pathway intermediate or the product. Alternatively, HHA-CoA, 6-HH, HDO, 6ACA-CoA, 6ACA-SADH, CPO, or HMD also can be biosynthetically produced from cells through co-culture or co-fermentation using two different cells in the same vessel, where the first cell produces a HHA-CoA, 6-HH, HDO, 6ACA-CoA, 6ACA-SADH, CPO, or HMD intermediate and the second cell converts the intermediate to HHA-CoA, 6-HH, HDO, 6ACA-CoA, 6ACA-SADH, CPO, or HMD, or a downstream product related thereto such as an ester or amide thereof.

Given the teachings and guidance provided herein, those skilled in the art will understand that a wide variety of combinations and permutations exist for the non-naturally occurring cells and methods provided herein together with other cells, with the co-culture of other non-naturally occurring cells having subpathways and with combinations of other chemical and/or biochemical procedures well known in the art to produce HHA-CoA, 6-HH, HDO, 6ACA-CoA, 6ACA-SADH, CPO, or HMD, or a downstream product related thereto such as an ester or amide thereof.

Similarly, it is understood by those skilled in the art that a host organism can be selected based on desired characteristics for introduction of one or more gene disruptions to increase synthesis or production of HHA-CoA, 6-HH, HDO, 6ACA-CoA, 6ACA-SADH, CPO, or HMD, or a downstream product related thereto such as an ester or amide thereof. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods described herein can be applied to any suitable host microorganism to identify the cognate metabolic alterations needed to construct an organism in a species of interest that will increase biosynthesis of HHA-CoA, 6-HH, HDO, 6ACA-CoA, 6ACA-SADH, CPO, or HMD, or a downstream product related thereto such as an ester or amide thereof. In a particular embodiment, the increased production couples biosynthesis of HHA-CoA, 6-HH, HDO, 6ACA-CoA, 6ACA-SADH, CPO, or HMD, or a downstream product related thereto such as an ester or amide thereof to growth of the organism, and can obligatorily couple production of HHA-CoA, 6-HH, HDO, 6ACA-CoA, 6ACA-SADH, CPO, or HMD, or a downstream product related thereto such as an ester or amide thereof to growth of the organism if desired and as disclosed herein.

Sources of encoding nucleic acids for a HHA-CoA, 6-HH, HDO, 6ACA-CoA, 6ACA-SADH, CPO, or HMD pathway enzyme or protein, or a downstream product related thereto such as an ester or amide thereof, can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Saccharomyces cerevisiae, Saccharomyces kluyveri, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, Acidaminococcus fermentans, Acinetobacter baylyi, Acinetobacter calcoaceticus, Aquifex aeolicus, Arabidopsis thaliana, Archaeoglobus fulgidus, Aspergillus niger, Aspergillus terreus, Bacillus subtilis, Bos Taurus, Candida albicans, Candida tropicalis, Chlamydomonas reinhardtii, Chlorobium tepidum, Citrobacter koseri, Citrus junos, Clostridium acetobutylicum, Clostridium kluyveri, Clostridium saccharoperbutylacetonicum, Cyanobium PCC7001, Desulfatibacillum alkenivorans, Dictyostelium discoideum, Fusobacterium nucleatum, Haloarcula marismortui, Homo sapiens, Hydrogenobacter thermophilus, Klebsiella pneumoniae, Kluyveromyces lactis, Lactobacillus brevis, Leuconostoc mesenteroides, Metallosphaera sedula, Methanothermobacter thermautotrophicus, Mus musculus, Mycobacterium avium, Mycobacterium bovis, Mycobacterium marinum, Mycobacterium smegmatis, Nicotiana tabacum, Nocardia iowensis, Oryctolagus cuniculus, Penicillium chrysogenum, Pichia pastoris, Porphyromonas gingivalis, Porphyromonas gingivalis, Pseudomonas aeruginos, Pseudomonas putida, Pyrobaculum aerophilum, Ralstonia eutropha, Rattus norvegicus, Rhodobacter sphaeroides, Saccharomyces cerevisiae, Salmonella enteric, Salmonella typhimurium, Schizosaccharomyces pombe, Sulfolobus acidocaldarius, Sulfolobus solfataricus, Sulfolobus tokodaii, Thermoanaerobacter tengcongensis, Thermus thermophilus, Trypanosoma brucei, Tsukamurella paurometabola, Yarrowia lipolytica, Zoogloea ramigera and Zymomonas mobilis, Clostridum species, including but no limited to Clostridium saccharoperbutylacetonicum, Clostridium beijerinckii, Clostridium saccharobutylicum, Clostridium botulinum, Clostridium methylpentosum, Clostridium sticklandii, Clostridium phytofermentans, Clostridium saccharolyticum, Clostridium asparagiforme, Clostridium celatum, Clostridium carboxidivorans, Clostridium clostridioforme, Clostridium bolteae, Caldalkalibacillus thermarum, Clostridium botulinum, Pelosinus fermentans, Thermoanaerobacterium thermosaccharolyticum, Desulfosporosinus speices, Thermoanaerobacterium species, including but not limited to Thermoanaerobacterium saccharolyticum, Thermoanaerobacterium xylanolyticum, Acetonema longum, Geobacillus species, including but not limited to Geobacillus thermoglucosidans, Bacillus azotoformans, Thermincola potens, Fusobacterium species, including but not limited to Fusobacterium nucleatum, Fusobacterium ulcerans, Fusobacterium varium, Ruminococcus species, including but not limited to Ruminococcus gnavus, Ruminococcus obeum, Lachnospiraceae bacterium, Flavonfractor plautii, Roseburia inulinivorans, Acetobacterium woodii, Eubacterium species, including but not limited to Eubacterium plexicaudatum, Eubacterium hallii, Eubacterium limosum, Eubacterium yurii, Eubacteriaceae bacterium, Thermosediminibacter oceani, Ilyobacter polytropus, Shuttleworthia satelles, Halanaerobium saccharolyticum, Thermoanaerobacter ethanolicus, Rhodospirillum rubrum, Vibrio, Propionibacterium propionicum as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the HHA-CoA, 6-HH, HDO, 6ACA-CoA, 6ACA-SADH, CPO, or HMD biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of HHA-CoA, 6-HH, HDO, 6ACA-CoA, 6ACA-SADH, CPO, or HMD, or a downstream product related thereto such as an ester or amide thereof, including expression of an engineered ACL and/or engineered ALDH provided herein, described herein with reference to a particular organism such as E. coli can be readily applied to other cells such as microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative HHA-CoA, 6-HH, HDO, 6ACA-CoA, 6ACA-SADH, CPO, or HMD biosynthetic pathway exists in an unrelated species, HHA-CoA, 6-HH, HDO, 6ACA-CoA, 6ACA-SADH, CPO, or HMD biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods provided herein can be applied to all cells using the cognate metabolic alterations to those exemplified herein to construct a cell in a species of interest that will synthesize HHA-CoA, 6-HH, HDO, 6ACA-CoA, 6ACA-SADH, CPO, or HMD, or a downstream product related thereto such as an ester or amide thereof, if desired, including introducing an engineered ACL and/or engineered ALDH provided herein.

Methods for constructing and testing the expression levels of a non-naturally occurring host producing HHA-CoA, 6-HH, HDO, 6ACA-CoA, 6ACA-SADH, CPO, or HMD, or a downstream product related thereto such as an ester or amide thereof, including an engineered ACL and/or engineered ALDH provided herein, can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

A recombinant nucleic acid encoding an engineered ACL and/or engineered ALDH provided herein, and optionally exogenous nucleic acid sequences involved in a pathway for production of HHA-CoA, 6-HH, HDO, 6ACA-CoA, 6ACA-SADH, CPO, or HMD, or a downstream product related thereto such as an ester or amide thereof, can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include a recombinant nucleic acid encoding an engineered ACL and/or engineered ALDH provided herein, and/or optionally and/or an exogenous nucleic acid encoding a HHA-CoA, 6-HH, HDO, 6ACA-CoA, 6ACA-SADH, CPO, or HMD biosynthetic pathway, or nucleic acids encoding an enzyme that produces a downstream product related to HHA-CoA, 6-HH, HDO, 6ACA-CoA, 6ACA-SADH, CPO, or HMD such as an ester or amide thereof, as exemplified herein operably linked to expression control sequences functional in the host organism. Expression vectors applicable for use in the host cells provided herein include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences encoding an engineered ACL and/or engineered ALDH provided herein or encoding polypeptides involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

A vector or expression vector can also be used to express an encoded nucleic acid to produce an encoded polypeptide by in vitro transcription and translation. Such a vector or expression vector will comprise at least a promoter, and includes the vectors described herein above. Such a vector for in vitro transcription and translation generally is double stranded DNA. Methods of in vitro transcription and translation are well known to those skilled in the art (see Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). Kits for in vitro transcription and translation are also commercially available (see, for example, Promega, Madison, WI; New England Biolabs, Ipswich, MA; Thermo Fisher Scientific, Carlsbad, CA).

In some embodiments, provided herein is a non-naturally occurring microbial organism having a vector described herein comprising a nucleic acid described herein. Also provided a non-naturally occurring microbial organism having a nucleic acid described herein. In some embodiments, the nucleic acid is integrated into a chromosome of the organism. In some embodiments, the integration is site-specific. In an embodiment described herein, the nucleic acid is expressed. In some embodiments, provided herein is a non-naturally occurring microbial organism having an engineered ACL and/or engineered ALDH described herein.

In some embodiments, provided herein is a method for producing HHA-CoA, 6-HH, HDO, 6ACA-CoA, 6ACA-SADH, CPO, and/or HMD, or an ester or amide thereof, comprising culturing a cell provided herein under conditions and for a sufficient period of time to produce HHA-CoA, 6-HH, HDO, 6ACA-CoA, 6ACA-SADH, CPO, and/or HMD, or an ester or amide thereof. Such a cell expresses an engineered ACL and/or engineered ALDH provided herein. In one embodiment, the cell is in a substantially anaerobic culture medium. In some embodiments, the method can further comprise isolating or purifying the HHA-CoA, 6-HH, HDO, 6ACA-CoA, 6ACA-SADH, CPO, and/or HMD, or ester or amide thereof. In a particular embodiment, the isolating or purifying comprises distillation.

In one embodiment, provided herein a process for producing a product provided herein, comprising chemically reacting the HHA-CoA, 6-HH, HDO, 6ACA-CoA, 6ACA-SADH, CPO, and/or HMD, with itself or another compound in a reaction that produces the product.

In one embodiment, provided herein is a method for producing HHA-CoA, 6-HH, HDO, 6ACA-CoA, 6ACA-SADH, CPO, and/or HMD, or an ester or amide thereof, comprising providing a substrate to an engineered ACL and/or engineered ALDH provided herein and converting the substrate to HHA-CoA, 6-HH, HDO, 6ACA-CoA, 6ACA-SADH, CPO, and/or HMD, wherein the substrate is HHA and/or HHA-CoA. In one embodiment, the engineered ACL and/or engineered ALDH is present in a cell, in a cell lysate, or is isolated from a cell or cell lysate.

In one embodiment, provided herein is a method for producing HHA-CoA, 6-HH, HDO, 6ACA-CoA, 6ACA-SADH, CPO, and/or HMD, comprising incubating a lysate of a cell provided herein to produce HHA-CoA, 6-HH, HDO, 6ACA-CoA, 6ACA-SADH, CPO, and/or HMD. In one embodiment, the cell lysate is mixed with a second cell lysate, wherein the second cell lysate comprises an enzymatic activity to produce a substrate of an engineered ACL and/or engineered ALDH provided herein, or a downstream product of HHA-CoA, 6-HH, HDO, 6ACA-CoA, 6ACA-SADH, CPO, and/or HMD.

Also provided herein is a method for producing an engineered ACL and/or engineered ALDH provided herein, comprising expressing the engineered ACL and/or engineered ALDH in a cell. Still further provided herein is a method for producing an engineered ACL and/or engineered ALDH provided herein, comprising in vitro transcribing and translating a nucleic acid provided herein or a vector provided herein to produce the engineered ACL and/or engineered ALDH.

As described herein, a cell can be used to express an engineered ACL and/or engineered ALDH provided herein, and optionally the cell can include a metabolic pathway that utilizes an engineered ACL and/or engineered ALDH provided herein to produce a desired product, such as HHA-CoA, 6-HH, HDO, 6ACA-CoA, 6ACA-SADH, CPO, and/or HMD. Such methods for expressing a desired product are described herein. Alternatively, an engineered ACL and/or engineered ALDH provided herein can be expressed, and/or a desired product produced, in a cell lysate, for example, a cell lysate of a cell expressing an engineered ACL and/or engineered ALDH provided herein, or a cell expressing an engineered ACL and/or engineered ALDH provided herein and a metabolic pathway to produce a desired product, as described herein. In another embodiment, an engineered ACL and/or engineered ALDH provided herein can be expressed by in vitro transcription and translation, in which the ACL and/or engineered ALDH is produced in a cell free system. The ACL and/or engineered ALDH expressed by in vitro transcription and translation can be used to carry out a reaction in vitro. Optionally, other enzymes, or cell lysate(s) containing such enzymes, can be used to convert the product of the ACL and/or engineered ALDH enzymatic reaction to a desired downstream product in vitro.

Suitable purification and/or assays to test for the expression of an ACL and/or engineered ALDH, or for production of HHA-CoA, 6-HH, HDO, 6ACA-CoA, 6ACA-SADH, CPO, or HMD, or a downstream product related thereto such as an ester or amide thereof, including assays to test for ACL and/or engineered ALDH activity, can be performed using well known methods (see also Example). Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art (see also Example).

The HHA-CoA, 6-HH, HDO, 6ACA-CoA, 6ACA-SADH, CPO, or HMD, or other desired product, such as a downstream product related thereto such as an ester or amide thereof, can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, or ultrafiltration. All of the above methods are well known in the art.

Any of the non-naturally occurring cells expressing an engineered ACL and/or engineered ALDH provided herein described herein can be cultured to produce and/or secrete the biosynthetic products provided herein. For example, the cells that produce HHA-CoA, 6-HH, HDO, 6ACA-CoA, 6ACA-SADH, CPO, or HMD, or a downstream product related thereto such as an ester or amide thereof, can be cultured for the biosynthetic production of HHA-CoA, 6-HH, HDO, 6ACA-CoA, 6ACA-SADH, CPO, or HMD, or a downstream product related thereto such as an ester or amide thereof. Accordingly, in some embodiments, provided herein is a culture medium containing the HHA-CoA, 6-HH, HDO, 6ACA-CoA, 6ACA-SADH, CPO, or HMD, or a downstream product related thereto such as an ester or amide thereof, or HHA-CoA, 6-HH, HDO, 6ACA-CoA, 6ACA-SADH, CPO, or HMD pathway intermediate described herein. In some aspects, the culture medium can also be separated from the non-naturally occurring cells provided herein that produced the HHA-CoA, 6-HH, HDO, 6ACA-CoA, 6ACA-SADH, CPO, or HMD, or a downstream product related thereto such as an ester or amide thereof, or HHA-CoA, 6-HH, HDO, 6ACA-CoA, 6ACA-SADH, CPO, or HMD pathway intermediate. Methods for separating a cell from culture medium are well known in the art. Exemplary methods include filtration, flocculation, precipitation, centrifugation, sedimentation, and the like.

For the production of an engineered ACL and/or engineered ALDH provided herein, or of HHA-CoA, 6-HH, HDO, 6ACA-CoA, 6ACA-SADH, CPO, or HMD, or a downstream product related thereto such as an ester or amide thereof, in a cell expressing an engineered ACL and/or engineered ALDH provided herein, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United States publication 2009/0047719, filed Aug. 10, 2007. Fermentations can be performed in a batch, fed-batch or continuous manner, as disclosed herein. Fermentations can also be conducted in two phases, if desired. The first phase can be aerobic to allow for high growth and therefore high productivity, followed by an anaerobic phase of high yields of a desired product such as HHA-CoA, 6-HH, HDO, 6ACA-CoA, 6ACA-SADH, CPO, or HMD, or a downstream product related thereto such as an ester or amide thereof.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring cell. Such sources include, for example: sugars such as glucose, xylose, arabinose, galactose, mannose, fructose, sucrose and starch; or glycerol, and it is understood that a carbon source can be used alone as the sole source of carbon or in combination with other carbon sources described herein or known in the art. In one embodiment, the carbon source is a sugar. In one embodiment, the carbon source is a sugar-containing biomass. In some embodiments, the sugar is glucose. In one embodiment, the sugar is xylose. In another embodiment, the sugar is arabinose. In one embodiment, the sugar is galactose. In another embodiment, the sugar is fructose. In other embodiments, the sugar is sucrose. In one embodiment, the sugar is starch. In certain embodiments, the carbon source is glycerol. In some embodiments, the carbon source is crude glycerol. In one embodiment, the carbon source is crude glycerol without treatment. In other embodiments, the carbon source is glycerol and glucose. In another embodiment, the carbon source is methanol and glycerol. In one embodiment, the carbon source is carbon dioxide. In one embodiment, the carbon source is formate. In one embodiment, the carbon source is methane. In one embodiment, the carbon source is methanol. In certain embodiments, methanol is used alone as the sole source of carbon or in combination with other carbon sources described herein or known in the art. In a specific embodiment, the methanol is the only (sole) carbon source. In one embodiment, the carbon source is chemoelectro-generated carbon (see, e.g., Liao et al. (2012) Science 335:1596). In one embodiment, the chemoelectro-generated carbon is methanol. In one embodiment, the chemoelectro-generated carbon is formate. In one embodiment, the chemoelectro-generated carbon is formate and methanol. In one embodiment, the carbon source is a carbohydrate and methanol. In one embodiment, the carbon source is a sugar and methanol. In another embodiment, the carbon source is a sugar and glycerol. In other embodiments, the carbon source is a sugar and crude glycerol. In yet other embodiments, the carbon source is a sugar and crude glycerol without treatment. In one embodiment, the carbon source is a sugar-containing biomass and methanol. In another embodiment, the carbon source is a sugar-containing biomass and glycerol. In other embodiments, the carbon source is a sugar-containing biomass and crude glycerol. In yet other embodiments, the carbon source is a sugar-containing biomass and crude glycerol without treatment. In some embodiments, the carbon source is a sugar-containing biomass, methanol and a carbohydrate. Other sources of carbohydrate include, for example, renewable feedstocks and biomass. Exemplary types of biomasses that can be used as feedstocks in the methods provided herein include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the cells provided herein for the expression of an engineered ACL and/or engineered ALDH provided herein, and optionally production of HHA-CoA, 6-HH, HDO, 6ACA-CoA, 6ACA-SADH, CPO, or HMD, or a downstream product thereof, such as an ester or amide thereof.

In addition to renewable feedstocks such as those exemplified above, the cells provided herein that produce HHA-CoA, 6-HH, HDO, 6ACA-CoA, 6ACA-SADH, CPO, or HMD or a downstream product thereof, such as an ester or amide thereof, also can be modified for growth on syngas as its source of carbon. In this specific embodiment, one or more proteins or enzymes are expressed in the HHA-CoA, 6-HH, HDO, 6ACA-CoA, 6ACA-SADH, CPO, or HMD producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

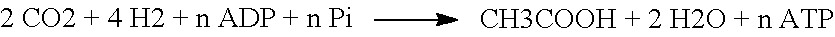

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-TiF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC)(see WO2009/094485). Following the teachings and guidance provided herein for introducing a sufficient number of encoding nucleic acids to generate a HHA-CoA, 6-HH, HDO, 6ACA-CoA, 6ACA-SADH, CPO, or HMD pathway, or a downstream product related thereto such as an ester or amide thereof, including a nucleic acid encoding an engineered ACL and/or engineered ALDH provided herein, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the cells provided herein such that the modified organism contains the complete Wood-Ljungdahl pathway will confer syngas utilization ability.

Additionally, the reductive (reverse) tricarboxylic acid cycle coupled with carbon monoxide dehydrogenase and/or hydrogenase activities can also be used for the conversion of CO, CO₂ and/or H₂ to acetyl-CoA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate:ferredoxin oxidoreductase, succinyl-CoA synthetase, succinyl-CoA transferase, fumarate reductase, fumarase, malate dehydrogenase, NAD(P) H:ferredoxin oxidoreductase, carbon monoxide dehydrogenase, and hydrogenase. Specifically, the reducing equivalents extracted from CO and/or H₂ by carbon monoxide dehydrogenase and hydrogenase are utilized to fix CO₂ via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to glyceraldehyde-3-phosphate, phosphoenolpyruvate, and pyruvate, by pyruvate:ferredoxin oxidoreductase and the enzymes of gluconeogenesis. Acetyl-CoA can also be converted to 3-oxoadipyl-CoA by, for example, beta-ketothiolase to funnel into a HDO pathway, as disclosed herein (see FIG. 1). Following the teachings and guidance provided herein for introducing a sufficient number of encoding nucleic acids to generate a HHA-CoA, 6-HH, HDO, 6ACA-CoA, 6ACA-SADH, CPO, or HMD pathway, or pathway to generate a downstream product related thereto such as an ester or amide thereof, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the cells provided herein can be performed such that the modified organism contains a reductive TCA pathway.

Accordingly, given the teachings and guidance provided herein, those skilled in the art will understand that a non-naturally occurring cell can be produced that produces and/or secretes the biosynthesized compounds provided herein when grown on a carbon source such as a carbohydrate. Such compounds include, for example, HHA-CoA, 6-HH, HDO, 6ACA-CoA, 6ACA-SADH, CPO, or HMD, or a downstream product related thereto such as an ester or amide thereof, and any of the intermediate metabolites in the HHA-CoA, 6-HH, HDO, 6ACA-CoA, 6ACA-SADH, CPO, or HMD pathway. All that is required is to engineer in one or more of the required enzyme or protein activities to achieve biosynthesis of the desired compound or intermediate including, for example, inclusion of some or all of the biosynthetic pathways for HHA-CoA, 6-HH, HDO, 6ACA-CoA, 6ACA-SADH, CPO, or HMD, or a downstream product related thereto such as an ester or amide thereof, including an engineered ACL and/or engineered ALDH provided herein. Accordingly, provided herein is a non-naturally occurring cell that produces and/or secretes HHA-CoA, 6-HH, HDO, 6ACA-CoA, 6ACA-SADH, CPO, or HMD, or a downstream product related thereto such as an ester or amide thereof, when grown on a carbohydrate or other carbon source and produces and/or secretes any of the intermediate metabolites shown in the HHA-CoA, 6-HH, HDO, 6ACA-CoA, 6ACA-SADH, CPO, or HMD pathway when grown on a carbohydrate or other carbon source. The cells producing HHA-CoA, 6-HH, HDO, 6ACA-CoA, 6ACA-SADH, CPO, or HMD, or a downstream product related thereto such as an ester or amide thereof, provided herein can initiate synthesis from an intermediate of a HHA-CoA, 6-HH, HDO, 6ACA-CoA, 6ACA-SADH, CPO, or HMD pathway.

The non-naturally occurring cells provided herein are constructed using methods well known in the art as exemplified herein to exogenously express an engineered ACL and/or engineered ALDH provided herein, and optionally at least one nucleic acid encoding a HHA-CoA, 6-HH, HDO, 6ACA-CoA, 6ACA-SADH, CPO, or HMD pathway enzyme or protein, or a downstream product related thereto such as an ester or amide thereof. The enzymes or proteins can be expressed in sufficient amounts to produce HHA-CoA, 6-HH, HDO, 6ACA-CoA, 6ACA-SADH, CPO, or HMD, or a downstream product related thereto such as an ester or amide thereof. It is understood that the cells provided herein are cultured under conditions sufficient to express an engineered ACL and/or engineered ALDH provided herein or produce HHA-CoA, 6-HH, HDO, 6ACA-CoA, 6ACA-SADH, CPO, or HMD, or a downstream product related thereto such as an ester or amide thereof. Following the teachings and guidance provided herein, the non-naturally occurring cells provided herein can achieve biosynthesis of HHA-CoA, 6-HH, HDO, 6ACA-CoA, 6ACA-SADH, CPO, or HMD, or a downstream product related thereto such as an ester or amide thereof, resulting in intracellular concentrations between about 0.1-300 mM or more, for example, 0.1-1.3 M or higher. Generally, the intracellular concentration of HHA-CoA, 6-HH, HDO, 6ACA-CoA, 6ACA-SADH, CPO, or HMD, or a downstream product related thereto such as an ester or amide thereof, is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring cells provided herein. For example, the intracellular concentration of HHA-CoA, 6-HH, HDO, 6ACA-CoA, 6ACA-SADH, CPO, or HMD, or a downstream product related thereto such as an ester or amide thereof, can be between about 100 mM to 1.3 M, including about 100 mM, 200 mM, 500 mM, 800 mM, 1 M, 1.1 M, 1.2 M, 1.3 M, or higher.

A cell provided herein is cultured using well known methods. The culture conditions can include, for example, liquid culture procedures as well as fermentation and other large scale culture procedures. As described herein, particularly useful yields of the biosynthetic products provided herein can be obtained under anaerobic or substantially anaerobic culture conditions.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring cells as well as other anaerobic conditions well known in the art. Under such anaerobic or substantially anaerobic conditions, the HHA-CoA, 6-HH, HDO, 6ACA-CoA, 6ACA-SADH, CPO, or HMD producers can synthesize HHA-CoA, 6-HH, HDO, 6ACA-CoA, 6ACA-SADH, CPO, or HMD, or a downstream product related thereto such as an ester or amide thereof, at intracellular concentrations of 5-10 mM or more as well as all other concentrations exemplified herein. It is understood that, even though the above description refers to intracellular concentrations, HHA-CoA, 6-HH, HDO, 6ACA-CoA, 6ACA-SADH, CPO, or HMD producing cells can produce HHA-CoA, 6-HH, HDO, 6ACA-CoA, 6ACA-SADH, CPO, or HMD, or a downstream product related thereto such as an ester or amide thereof, intracellularly and/or secrete the product into the culture medium.

As described herein, one exemplary growth condition for achieving biosynthesis of HHA-CoA, 6-HH, HDO, 6ACA-CoA, 6ACA-SADH, CPO, or HMD, or a downstream product related thereto such as an ester or amide thereof, includes anaerobic culture or fermentation conditions. In certain embodiments, the non-naturally occurring cells provided herein can be sustained, cultured or fermented under anaerobic or substantially anaerobic conditions. Briefly, an anaerobic condition refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of HHA-CoA, 6-HH, HDO, 6ACA-CoA, 6ACA-SADH, CPO, or HMD, or a downstream product related thereto such as an ester or amide thereof, by a cell provided herein. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of HHA-CoA, 6-HH, HDO, 6ACA-CoA, 6ACA-SADH, CPO, or HMD, or a downstream product related thereto such as an ester or amide thereof. Generally, and as with non-continuous culture procedures, the continuous and/or near-continuous production of HHA-CoA, 6-HH, HDO, 6ACA-CoA, 6ACA-SADH, CPO, or HMD, or a downstream product related thereto such as an ester or amide thereof, will include culturing a non-naturally occurring cell producing HHA-CoA, 6-HH, HDO, 6ACA-CoA, 6ACA-SADH, CPO, or HMD, or a downstream product related thereto such as an ester or amide thereof, provided herein in sufficient nutrients and medium to sustain and/or nearly sustain growth in an exponential phase. Continuous culture under such conditions can include, for example, growth or culturing for 1 day, 2, 3, 4, 5, 6 or 7 days or more. Additionally, continuous culture can include longer time periods of 1 week, 2, 3, 4 or 5 or more weeks and up to several months. Alternatively, organisms provided herein can be cultured for hours, if suitable for a particular application. It is to be understood that the continuous and/or near-continuous culture conditions also can include all time intervals in between these exemplary periods. It is further understood that the time of culturing the cell provided herein is for a sufficient period of time to produce a sufficient amount of product for a desired purpose.

Exemplary fermentation processes include, but are not limited to, fed-batch fermentation and batch separation; fed-batch fermentation and continuous separation; and continuous fermentation and continuous separation. In an exemplary batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, product concentration, and the like. In a batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. Upon completion of the cultivation period, the fermenter contents can be passed through a cell separation unit, for example, a centrifuge, filtration unit, and the like, to remove cells and cell debris. In the case where the desired product is expressed intracellularly, the cells can be lysed or disrupted enzymatically or chemically prior to or after separation of cells from the fermentation broth, as desired, in order to release additional product. The fermentation broth can be transferred to a product separations unit. Isolation of product occurs by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like) to provide an organic solution of the product, if appropriate, standard distillation methods, and the like, depending on the chemical characteristics of the product of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, and fermentation liquid is withdrawn at the same rate. Under such conditions, the product concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of product concentration and/or cell density. In continuous mode, fermenter contents are constantly removed as new feed medium is supplied. The exit stream, containing cells, medium, and product, are generally subjected to a continuous product separations procedure, with or without removing cells and cell debris, as desired. Continuous separations methods employed in the art can be used to separate the product from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like), standard continuous distillation methods, and the like, or other methods well known in the art.

Fermentation procedures are well known in the art. Briefly, fermentation for the biosynthetic production of HHA-CoA, 6-HH, HDO, 6ACA-CoA, 6ACA-SADH, CPO, or HMD, or a downstream product related thereto such as an ester or amide thereof, can be utilized in, for example, fed-batch fermentation and batch separation; fed-batch fermentation and continuous separation, or continuous fermentation and continuous separation. Examples of batch and continuous fermentation procedures are well known in the art and described herein.

In addition to the fermentation procedures described herein using the producers of HHA-CoA, 6-HH, HDO, 6ACA-CoA, 6ACA-SADH, CPO, or HMD, or a downstream product related thereto such as an ester or amide thereof, provided herein for continuous production of substantial quantities of HHA-CoA, 6-HH, HDO, 6ACA-CoA, 6ACA-SADH, CPO, or HMD, or a downstream product related thereto such as an ester or amide thereof, the HHA-CoA, 6-HH, HDO, 6ACA-CoA, 6ACA-SADH, CPO, or HMD, or a downstream product related thereto such as an ester or amide, producers also can be, for example, simultaneously subjected to chemical synthesis and/or enzymatic procedures to convert the product to other compounds, or the product can be separated from the fermentation culture and sequentially subjected to chemical and/or enzymatic conversion to convert the product to other compounds, if desired.

In some embodiments, the carbon feedstock and other cellular uptake sources such as phosphate, ammonia, sulfate, chloride and other halogens can be chosen to alter the isotopic distribution of the atoms present in HHA-CoA, 6-HH, HDO, 6ACA-CoA, 6ACA-SADH, CPO, or HMD, or a downstream product related thereto such as an ester or amide thereof, or any HHA-CoA, 6-HH, HDO, 6ACA-CoA, 6ACA-SADH, CPO, or HMD pathway intermediate. The various carbon feedstock and other uptake sources enumerated above will be referred to herein, collectively, as “uptake sources.” Uptake sources can provide isotopic enrichment for any atom present in the product HHA-CoA, 6-HH, HDO, 6ACA-CoA, 6ACA-SADH, CPO, or HMD, or a downstream product related thereto such as an ester or amide thereof, or HHA-CoA, 6-HH, HDO, 6ACA-CoA, 6ACA-SADH, CPO, or HMD pathway intermediate, or for side products generated in reactions diverging away from a HHA-CoA, 6-HH, HDO, 6ACA-CoA, 6ACA-SADH, CPO, or HMD pathway. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased source derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

In the case of carbon, ASTM D6866 was developed in the United States as a standardized analytical method for determining the biobased content of solid, liquid, and gaseous samples using radiocarbon dating by the American Society for Testing and Materials (ASTM) International. The standard is based on the use of radiocarbon dating for the determination of a product's biobased content. ASTM D6866 was first published in 2004, and the current active version of the standard is ASTM D6866-11 (effective Apr. 1, 2011). Radiocarbon dating techniques are well known to those skilled in the art, including those described herein.

The biobased content of a compound is estimated by the ratio of carbon-14 (¹⁴C) to carbon-12 (¹²C). Specifically, the Fraction Modern (Fm) is computed from the expression: Fm =(S−B)/(M−B), where B, S and M represent the ¹⁴C/¹²C ratios of the blank, the sample and the modern reference, respectively. Fraction Modern is a measurement of the deviation of the ¹⁴C/¹²C ratio of a sample from “Modern.” Modem is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil (Olsson, The use of Oxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950)¹⁴C/¹²C ratio of 1.176±0.010×10⁻¹² (Karlen et al., Arkiv Geofysik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for δ¹³.

An oxalic acid standard (SRM 4990b or HOx 1) was made from a crop of 1955 sugar beet. Although there were 1000 lbs made, this oxalic acid standard is no longer commercially available. The Oxalic Acid II standard (HOx 2; N.I.S.T designation SRM 4990 C) was made from a crop of 1977 French beet molasses. In the early 1980's, a group of 12 laboratories measured the ratios of the two standards. The ratio of the activity of Oxalic acid II to 1 is 1.2933±0.001 (the weighted mean). The isotopic ratio of HOx II is −17.8 per mil. ASTM D6866-11 suggests use of the available Oxalic Acid II standard SRM 4990 C (Hox2) for the modern standard (see discussion of original vs. currently available oxalic acid standards in Mann, Radiocarbon, 25(2):519-527 (1983)). A Fm=0% represents the entire lack of carbon-14 atoms in a material, thus indicating a fossil (for example, petroleum based) carbon source. A Fm=100%, after correction for the post-1950 injection of carbon-14 into the atmosphere from nuclear bomb testing, indicates an entirely modern carbon source. As described herein, such a “modern” source includes biobased sources.

As described in ASTM D6866, the percent modern carbon (pMC) can be greater than 100% because of the continuing but diminishing effects of the 1950s nuclear testing programs, which resulted in a considerable enrichment of carbon-14 in the atmosphere as described in ASTM D6866-11. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old.

ASTM D6866 quantifies the biobased content relative to the material's total organic content and does not consider the inorganic carbon and other non-carbon containing substances present. For example, a product that is 50% starch-based material and 50% water would be considered to have a Biobased Content=100% (50% organic content that is 100% biobased) based on ASTM D6866. In another example, a product that is 50% starch-based material, 25% petroleum-based, and 25% water would have a Biobased Content=66.7% (75% organic content but only 50% of the product is biobased). In another example, a product that is 50% organic carbon and is a petroleum-based product would be considered to have a Biobased Content=0% (50% organic carbon but from fossil sources). Thus, based on the well known methods and known standards for determining the biobased content of a compound or material, one skilled in the art can readily determine the biobased content of a compound or material and/or prepared downstream products that utilize a compound or material provided herein having a desired biobased content.

Applications of carbon-14 dating techniques to quantify bio-based content of materials are known in the art (Currie et al., Nuclear Instruments and Methods in Physics Research B, 172:281-287 (2000)). For example, carbon-14 dating has been used to quantify bio-based content in terephthalate-containing materials (Colonna et al., Green Chemistry, 13:2543-2548 (2011)). Notably, polypropylene terephthalate (PPT) polymers derived from renewable 1,3-propanediol and petroleum-derived terephthalic acid resulted in Fm values near 30% (i.e., since 3/11 of the polymeric carbon derives from renewable 1,3-propanediol and 8/11 from the fossil end member terephthalic acid) (Currie et al., supra, 2000). In contrast, polybutylene terephthalate polymer derived from both renewable 1,4-butanediol and renewable terephthalic acid resulted in bio-based content exceeding 90% (Colonna et al., supra, 2011).

Accordingly, in some embodiments, provided herein is HHA-CoA, 6-HH, HDO, 6ACA-CoA, 6ACA-SADH, CPO, or HMD or a downstream product related thereto such as an ester or amide thereof, or a HHA-CoA, 6-HH, HDO, 6ACA-CoA, 6ACA-SADH, CPO, or HMD pathway intermediate, produced by a cell provided herein, that has a carbon-12, carbon-13, and carbon-14 ratio that reflects an atmospheric carbon, also referred to as environmental carbon, uptake source. For example, in some aspects the HHA-CoA, 6-HH, HDO, 6ACA-CoA, 6ACA-SADH, CPO, or HMD, or a downstream product related thereto such as an ester or amide thereof, or a HHA-CoA, 6-HH, HDO, 6ACA-CoA, 6ACA-SADH, CPO, or HMD pathway intermediate can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, provided herein is HHA-CoA, 6-HH, HDO, 6ACA-CoA, 6ACA-SADH, CPO, or HMD, or a downstream product related thereto such as an ester or amide thereof, or a HHA-CoA, 6-HH, HDO, 6ACA-CoA, 6ACA-SADH, CPO, or HMD pathway intermediate that has a carbon-12, carbon-13, and carbon-14 ratio that reflects petroleum-based carbon uptake source. In this aspect, the HHA-CoA, 6-HH, HDO, 6ACA-CoA, 6ACA-SADH, CPO, or HMD, or a downstream product related thereto such as an ester or amide thereof, or a HHA-CoA, 6-HH, HDO, 6ACA-CoA, 6ACA-SADH, CPO, or HMD pathway intermediate can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, provided herein is HHA-CoA, 6-HH, HDO, 6ACA-CoA, 6ACA-SADH, CPO, or HMD, or a downstream product related thereto such as an ester or amide thereof, or a HHA-CoA, 6-HH, HDO, 6ACA-CoA, 6ACA-SADH, CPO, or HMD pathway intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the compositions provided herein relate to the biologically produced HHA-CoA, 6-HH, HDO, 6ACA-CoA, 6ACA-SADH, CPO, or HMD, or a downstream product related thereto such as an ester or amide thereof, or HHA-CoA, 6-HH, HDO, 6ACA-CoA, 6ACA-SADH, CPO, or HMID pathway intermediate as disclosed herein, and to the products derived therefrom, wherein the HHA-CoA, 6-HH, HDO, 6ACA-CoA, 6ACA-SADH, CPO, or HMD, or a downstream product related thereto such as an ester or amide thereof, or a HHA-CoA, 6-HH, HDO, 6ACA-CoA, 6ACA-SADH, CPO, or HMD pathway intermediate has a carbon-12, carbon-13, and carbon-14 isotope ratio of about the same value as the CO₂ that occurs in the environment. For example, in some aspects provided herein is bioderived HHA-CoA, 6-HH, HDO, 6ACA-CoA, 6ACA-SADH, CPO, or HMD, or a downstream product related thereto such as an ester or amide thereof, or a bioderived HHA-CoA, 6-HH, HDO, 6ACA-CoA, 6ACA-SADH, CPO, or HMD intermediate having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from bioderived HHA-CoA, 6-HH, HDO, 6ACA-CoA, 6ACA-SADH, CPO, or HMD, or a downstream product related thereto such as an ester or amide thereof, or a bioderived HHA-CoA, 6-HH, HDO, 6ACA-CoA, 6ACA-SADH, CPO, or HMD pathway intermediate as disclosed herein, wherein the bioderived product is chemically modified to generate a final product. Methods of chemically modifying a bioderived product of HHA-CoA, 6-HH, HDO, 6ACA-CoA, 6ACA-SADH, CPO, or HMD, or a downstream product related thereto such as an ester or amide thereof, or an intermediate of a HHA-CoA, 6-HH, HDO, 6ACA-CoA, 6ACA-SADH, CPO, or HMD, to generate a desired product are well known to those skilled in the art, as described herein.

Further provided herein is plastics, polyurethanes, polyesters, nylons, organic solvents, reactive diluents, such as for UV-coatings, polyurethane resins, polyester resins, polyesterpolyol, polyesterols, polyester coatings, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products, which can be based on HHA-CoA, 6-HH, HDO, 6ACA-CoA, 6ACA-SADH, CPO, and/or HMD, or a downstream product related thereto such as an ester or amide thereof, and plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products, and the like, which can be based on HHA-CoA, 6-HH, HDO, 6ACA-CoA, 6ACA-SADH, CPO, or HMD, or a downstream product related thereto such as an ester or amide thereof, having a carbon-12 versus carbon-13 versus carbon-14 isotope ratio of about the same value as the CO₂ that occurs in the environment, wherein the plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products are generated directly from or in combination with bioderived HHA-CoA, 6-HH, HDO, 6ACA-CoA, 6ACA-SADH, CPO, or HMD, or a downstream product related thereto such as an ester or amide thereof, or a bioderived HHA-CoA, 6-HH, HDO, 6ACA-CoA, 6ACA-SADH, CPO, or HMD pathway intermediate as disclosed herein.

Thus, the engineered ACL and/or engineered ALDH provided herein is particularly useful to provide an improved enzymatic route and microorganism to provide an improved composition of HDO, and further having improved purity qualities with respect to by-products. 1,6-Hexanediol is raw material for polyurethane coatings, polyester resins and polycarbonatediol resins. It is also used as a raw material to make reactive diluent for epoxy resin. The chemical structure of 1,6-hexanediol, which contains terminally located hydroxyl groups, makes it highly reactive and useful for the manufacture of a variety of derivatives. It is also used as a raw material for a UV curing material. HDO has further consumer applications including use in the manufacture of ink, toner, and colorant products as well as for paint and coatings production. HDO is used in the pharmaceutical and research industry as a tool to probe liquid-liquid phase separation (LLPS) in cells. HDO finds use as an anti-microbial agent and preservative, and for example, preserves against spoilage by microorganisms. 1,6-Hexandiol is used as an intermediate in the manufacture of acrylics, adhesives, and dyestuffs. Styrene, maleic anhydride, fumaric acid, and unsaturated polyester resins have also been made from 1,6-hexanediol. HDO can be used at concentrations from 0.1% to 50%, and even less than 0.1% and even more than 50%, up to 100%.

In one embodiment, provided herein is a culture medium comprising bioderived HHA-CoA, 6-HH, HDO, 6ACA-CoA, 6ACA-SADH, CPO, and/or HMD wherein the bioderived HHA-CoA, 6-HH, HDO, 6ACA-CoA, 6ACA-SADH, CPO, and/or HMD, has a carbon-12, carbon-13 and carbon-14 isotope ratio that reflects an atmospheric carbon dioxide uptake source, and wherein the bioderived HHA-CoA, 6-HH, HDO, 6ACA-CoA, 6ACA-SADH, CPO, and/or HMD is produced by a cell, or in a cell lysate, provided herein or a method provided herein. In one embodiment, the culture medium is separated from the cell.

In one embodiment, provided herein is HHA-CoA, 6-HH, HDO, 6ACA-CoA, 6ACA-SADH, CPO, and/or HMD, having a carbon-12, carbon-13 and carbon-14 isotope ratio that reflects an atmospheric carbon dioxide uptake source, wherein the HHA-CoA, 6-HH, HDO, 6ACA-CoA, 6ACA-SADH, CPO, and/or HMD, is produced by a cell, or in a cell lysate, provided herein or a method provided herein. In one embodiment, the HHA-CoA, 6-HH, HDO, 6ACA-CoA, 6ACA-SADH, CPO, and/or HMD, has an Fm value of at least 80%, at least 85%, at least 90%, at least 95% or at least 98%.

In one embodiment, provided herein is HHA-CoA, 6-HH, HDO, 6ACA-CoA, 6ACA-SADH, CPO, and/or HMD, produced by a cell, or in a cell lysate provided herein or a method provided herein. In one embodiment, provided herein is HHA-CoA, 6-HH, HDO, 6ACA-CoA, 6ACA-SADH, CPO, and/or HMD having a carbon-12, carbon-13 and carbon-14 isotope ratio that reflects an atmospheric carbon dioxide uptake source, wherein the HHA-CoA, 6-HH, HDO, 6ACA-CoA, 6ACA-SADH, CPO, and/or HMD is produced by a cell, or in a cell lysate, provided herein or a method provided herein. In one embodiment, the HHA-CoA, 6-HH, HDO, 6ACA-CoA, 6ACA-SADH, CPO, and/or HMD has an Fm value of at least 80%, at least 85%, at least 90%, at least 95% or at least 98%.

In one embodiment, provided herein is a composition comprising HHA-CoA, 6-HH, HDO, 6ACA-CoA, 6ACA-SADH, CPO, and/or HMD, produced by a cell, or in a cell lysate, provided herein or a method provided herein and a compound other than the HHA-CoA, 6-HH, HDO, 6ACA-CoA, 6ACA-SADH, CPO, and/or HMD, respectively. In one embodiment, the compound other than the HHA-CoA, 6-HH, HDO, 6ACA-CoA, 6ACA-SADH, CPO, and/or HMD is a portion of a cell that produces the HHA-CoA, 6-HH, HDO, 6ACA-CoA, 6ACA-SADH, CPO, and/or HMD respectively, or that expresses an engineered ACL and/or engineered ALDH provided herein.

In one embodiment, provided herein is a composition comprising HHA-CoA, 6-HH, HDO, 6ACA-CoA, 6ACA-SADH, CPO, and/or HMD produced by a cell, or in a cell lysate, provided herein or a method provided herein, or a cell lysate or culture supernatant of a cell producing the HHA-CoA, 6-HH, HDO, 6ACA-CoA, 6ACA-SADH, CPO, and/or HMD.

In one embodiment, provided herein is a product comprising HHA-CoA, 6-HH, HDO, 6ACA-CoA, 6ACA-SADH, CPO, and/or HMD produced by a cell, or in a cell lysate provided herein or a method provided herein, wherein the product is a plastic, polyurethane, polyester, polyesterpolyol, polyesterol, polyester coating, nylon, organic solvent, reactive diluent, such as for UV-coatings, polyurethane resin, polyester resin, acrylic or acrylate, such as diacrylate, adhesive, such as co-terephthalate and urethane, plasticizer, such as for polyvinyl chloride, polymeric thickener, sizing agent, surfactant, dyestuffs, pesticide, and/or a hexamethylenediamine based product. In one embodiment, the product comprises at least 0.1%, at least 0.5%, at least 1%, at least 5%, at least 10%, at least 20%, at least 30%, at least 40% or at least 50% bioderived HHA-CoA, 6-HH, HDO, 6ACA-CoA, 6ACA-SADH, CPO, and/or HMD. In one embodiment, the product comprises a portion of the produced HHA-CoA, 6-HH, HDO, 6ACA-CoA, 6ACA-SADH, CPO, and/or HMD, as a repeating unit. In one embodiment, provided herein is a molded product obtained by molding a product made with or derived from HHA-CoA, 6-HH, HDO, 6ACA-CoA, 6ACA-SADH, CPO, and/or HMD produced by a cell, or in a cell lysate provided herein or a method provided herein.

Further provided herein is a composition comprising bioderived HHA-CoA, 6-HH, HDO, 6ACA-CoA, 6ACA-SADH, CPO, or HMD, or a downstream product related thereto such as an ester or amide thereof, and a compound other than the bioderived HHA-CoA, 6-HH, HDO, 6ACA-CoA, 6ACA-SADH, CPO, or HMD, or a downstream product related thereto such as an ester or amide thereof. The compound other than the bioderived product can be a cellular portion, for example, a trace amount of a cellular portion of, or can be fermentation broth or culture medium or a purified or partially purified fraction thereof produced in the presence of, a non-naturally occurring cell provided herein having a pathway that produces HHA-CoA, 6-HH, HDO, 6ACA-CoA, 6ACA-SADH, CPO, or HMD, or a downstream product related thereto such as an ester or amide thereof. The composition can comprise, for example, a reduced level of a byproduct when produced by an organism having reduced byproduct formation, as disclosed herein. The composition can comprise, for example, bioderived HHA-CoA, 6-HH, HDO, 6ACA-CoA, 6ACA-SADH, CPO, or HMD, or a downstream product related thereto such as an ester or amide thereof, or a cell lysate or culture supernatant of a cell provided herein.

HHA-CoA, 6-HH, HDO, 6ACA-CoA, 6ACA-SADH, CPO, or HMD, or a downstream product related thereto such as an ester or amide thereof, is a chemical used in commercial and industrial applications. Non-limiting examples of such applications include production of plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products. Moreover, HHA-CoA, 6-HH, HDO, 6ACA-CoA, 6ACA-SADH, CPO, or HMD is also used as a raw material in the production of a wide range of products including plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products. Accordingly, in some embodiments, provided herein is biobased plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products comprising one or more bioderived HHA-CoA, 6-HH, HDO, 6ACA-CoA, 6ACA-SADH, CPO, or HMD, or a downstream product related thereto such as an ester or amide thereof, or bioderived HHA-CoA, 6-HH, HDO, 6ACA-CoA, 6ACA-SADH, CPO, or HMD pathway intermediate produced by a non-naturally occurring cell provided herein, for example, expressing an engineered ACL and/or engineered ALDH provided herein, or produced using a method disclosed herein.

In some embodiments, provided herein is plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products comprising bioderived HHA-CoA, 6-HH, HDO, 6ACA-CoA, 6ACA-SADH, CPO, or HMD, or a downstream product related thereto such as an ester or amide thereof, or bioderived HHA-CoA, 6-HH, HDO, 6ACA-CoA, 6ACA-SADH, CPO, or HMD pathway intermediate, wherein the bioderived HHA-CoA, 6-HH, HDO, 6ACA-CoA, 6ACA-SADH, CPO, or HMD, or a downstream product related thereto such as an ester or amide thereof, or bioderived HHA-CoA, 6-HH, HDO, 6ACA-CoA, 6ACA-SADH, CPO, or HMD pathway intermediate includes all or part of the HHA-CoA, 6-HH, HDO, 6ACA-CoA, 6ACA-SADH, CPO, or HMD, or a downstream product related thereto such as an ester or amide thereof, or HHA-CoA, 6-HH, HDO, 6ACA-CoA, 6ACA-SADH, CPO, or HMD pathway intermediate used in the production of plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products. For example, the final plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products can contain the bioderived HHA-CoA, 6-HH, HDO, 6ACA-CoA, 6ACA-SADH, CPO, or HMD, or a downstream product related thereto such as an ester or amide thereof, or HHA-CoA, 6-HH, HDO, 6ACA-CoA, 6ACA-SADH, CPO, or HMD pathway intermediate, or a portion thereof that is the result of the manufacturing of plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products. Such manufacturing can include chemically reacting the bioderived HHA-CoA, 6-HH, HDO, 6ACA-CoA, 6ACA-SADH, CPO, or HMD, or a downstream product related thereto such as an ester or amide thereof, or bioderived HHA-CoA, 6-HH, HDO, 6ACA-CoA, 6ACA-SADH, CPO, or HMD pathway intermediate (e.g. chemical conversion, chemical functionalization, chemical coupling, oxidation, reduction, polymerization, copolymerization and the like) into the final plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products. Thus, in some aspects, provided herein is a biobased plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived HHA-CoA, 6-HH, HDO, 6ACA-CoA, 6ACA-SADH, CPO, or HMD, or a downstream product related thereto such as an ester or amide thereof, or bioderived HHA-CoA, 6-HH, HDO, 6ACA-CoA, 6ACA-SADH, CPO, or HMD pathway intermediate as disclosed herein.

Additionally, in some embodiments, provided herein is a composition having a bioderived HHA-CoA, 6-HH, HDO, 6ACA-CoA, 6ACA-SADH, CPO, or HMD, or a downstream product related thereto such as an ester or amide thereof, or HHA-CoA, 6-HH, HDO, 6ACA-CoA, 6ACA-SADH, CPO, or HMD pathway intermediate disclosed herein and a compound other than the bioderived HHA-CoA, 6-HH, HDO, 6ACA-CoA, 6ACA-SADH, CPO, or HMD, or a downstream product related thereto such as an ester or amide thereof, or HHA-CoA, 6-HH, HDO, 6ACA-CoA, 6ACA-SADH, CPO, or HMD pathway intermediate. For example, in some aspects, provided herein is biobased plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products wherein the HHA-CoA, 6-HH, HDO, 6ACA-CoA, 6ACA-SADH, CPO, or HMD, or a downstream product related thereto such as an ester or amide thereof, or HHA-CoA, 6-HH, HDO, 6ACA-CoA, 6ACA-SADH, CPO, or HMD pathway intermediate used in its production is a combination of bioderived and petroleum derived HHA-CoA, 6-HH, HDO, 6ACA-CoA, 6ACA-SADH, CPO, or HMD, or a downstream product related thereto such as an ester or amide thereof, or HHA-CoA, 6-HH, HDO, 6ACA-CoA, 6ACA-SADH, CPO, or HMD pathway intermediate. For example, biobased plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products can be produced using 50% bioderived HHA-CoA, 6-HH, HDO, 6ACA-CoA, 6ACA-SADH, CPO, or HMD, or a downstream product related thereto such as an ester or amide thereof, and 50% petroleum derived HHA-CoA, 6-HH, HDO, 6ACA-CoA, 6ACA-SADH, CPO, or HMD, or a downstream product related thereto such as an ester or amide thereof, or other desired ratios such as 60%/40%, 70%/30%, 80%/20%, 90%/10%, 95%/5%, 100%/0%, 40%/60%, 30%/70%, 20%/80%, 10%/90% of bioderived/petroleum derived precursors, so long as at least a portion of the product comprises a bioderived product produced by the cells disclosed herein. It is understood that methods for producing plastics, polyurethanes, polyesters, polyesterpolyol, polyesterols, polyester coatings, nylons, organic solvents, reactive diluents, such as for UV-coatings, polyurethane resins, polyester resins, acrylics or acrylates, such as diacrylate, adhesives, such as co-terephthalates and urethanes, plasticizers, such as for polyvinyl chloride, polymeric thickeners, sizing agents, surfactants, dyestuffs, pesticides, and/or hexamethylenediamine based products using the bioderived HHA-CoA, 6-HH, HDO, 6ACA-CoA, 6ACA-SADH, CPO, or HMD, or a downstream product related thereto such as an ester or amide thereof, or bioderived HHA-CoA, 6-HH, HDO, 6ACA-CoA, 6ACA-SADH, CPO, or HMD pathway intermediate provided herein are well known in the art.

### Sequences

The sequences in the following TABLE 1 illustrate template amino acid sequences that can be used to generate the ACL variant sequences and/or compositions and perform the methods described herein. As needed, a nucleotide sequence can be readily deduced from the amino acid sequence.

TABLE 1
ACL TEMPLATE AMINO ACID SEQUENCES

| SEQ | | |
|---|---|---|
| ID | | |
| NO: | Description | Amino Acid |
| 1. | YALIOE20405p from | MTQIIHNATIPNIPVDQLYDGKITDFIRSGGHSNETKPSVIDAKTGQT |
| | Yarrowia lipolytica | LSQAEMWQLSDKYAALLSSQYGLCRHRDNELDPSMGDVLITFFGNVIL |
| | CLIB122 (Accession | APVVHWAALDLGATISPGSTGYSAQDLAHQFRVTTPKVVVYAKAFKDV |
| | No. Q6C650) | VDEATKLYNSPNPPALVELEALDKQARMVGNHKVEHTRKIKLAPHESR |
| | | TRIAYLGMSSGTSGGVSKAVRLTHSNLTSCSEISNKASESLATDQQIA |
| | | AAIIPVSHLFGLSKFLIGNPHAGATTVYHNGEDLIEVLEAQKKYKVNS |
| | | WTLVPPIIVLLTKHPIVEKYIPSLRAHMRAILSGAAPLGANVTEALLT |
| | | RVTGNKFGESPEGGLRIVQGYGLTETSPVATLEDPEDKERHIRSCGKL |
| | | VPNSQVRIVNEDGVDQPAYDVDPNELDEAIKQGTLPVGELWIRGPQVM |
| | | DGYHNNPEANEACFVKADDAEADTAYYNRHWERTGDVALVDKQGRYMI |
| | | VDRTKEMIKSQGKQVAPAELEDMLLGHAQVADTAVIGIQDVEKGNEAP |
| | | RAFVVLKDPKYDAVEIKTWLDKQLPKYKQLHAGIVVIDAIPKNASGKI |
| | | LRRLLRARKDDVVLGLNKAKL |
| 2. | CoA ligase from | MNQIDASGIELLPHDVHQESRPDGSILLRSTHSLGAVAPNTGAWLHRW |
| | Actibacterium | AKEAPGRVAIAERAGAGWREHTYNELLNMVRNVAGALLARGLGADSPI |
| | lipolyticum | VVMSGNGVDHAILSLAAQYVGVPIVPLAEQYSLIPEAHGRLIYVLEKV |
| | (Accession No. | KPAMAFVSDAAQYASALSLPQLEGVELVAVKTDGAPVPVTAFADLLGH |
| | A0A238JIY9) | GQGGVDDAHAKVGPDTLAKILFTSGSSSDPKGVLTTHRMMCANQTQIS |
| | | DALRFLKARPPRILDWLPWNHVFGGSHNENMMLANGGSLYIDNGKPTK |
| | | AGFEDTLRNIKEHPGTLAFNVPVGYAMLVNAMERDPALRRAYFKELDL |
| | | IFYAGASLPQDVWESLERFAMEERGSLPLMASSWGMTETAPATIMVHE |
| | | PIGRSGVIGVPLPGAEVKLLPDDEMRCELRVKGPNIMRGYENDPAKTA |
| | | EAFDDEGFLVTGDAVRFVDDADPSRGLVEDGRVSEDFKLLTGTWVQAG |
| | | VLRLDALKALAGLVQDVVICGHDRDQIGLFIFPLPGAVSDTSAGAVID |
| | | PALSAQIEERLRDMAKTATGSAKRINRAIILSEPPQLKDHEITDKGSL |
| | | NIRKILTRRADLLERLYDNEDPALIRV |

The sequences in the following TABLE 2 illustrates template amino acid sequences that can be used to generate the ALDH variant sequences and/or compositions and perform the methods described herein. As needed, a nucleotide sequence can be readily deduced from the amino acid sequence.

TABLE 2
ALDH TEMPLATE AMINO ACID SEQUENCE

| SEQ | | |
|---|---|---|
| ID | | |
| NO: | Description | Amino Acid Sequence |
| 3. | Aldehyde | MEQAVKDYLDKMVAASRIAQQEFATYPQETVDKAVRTVGKAIYDNAEL |
| | dehydrogenase from | LAHMAVDETKMGNYADKIAKCVNKSKSVWWRMKDKKSRGIIKRIPELG |
| | Acidaminococcus | LVEVAKPIGVIGCVAPTTNPVINVMQNAMCALKCGNSMIVSPHPRAKH |
| | massiliensis | SSVKTVEVINEALAALGMPKNLIQVITEPSMELSAGLMSAVDLCICTG |
| | (Accession No. | GPGLVKAAYSSGKPAIGVGQGNVQVLVDRDADLDQVAAMVIKGRTEDN |
| | WP_075579339) | GVLCTCEQNVICPEDKKEEMIAALKKHGAYYIGNSEDAAKLRDTAFPN |
| | | GGPVSKEYPGASVKKIAQLSGIQGIPEDAKVIVSCTRGYGKDEPLAKE |
| | | KLFPVLAFFTYDKWEDAIHIAKTNLEMEGIGHSVVIHSNTPEHIEAVA |
| | | EAIPVSRFAVNQVGGTNLGGAMDNGLNPTTTLGCGTWGNNSISENFTY |
| | | YHLMNLTRVSYRVPDMYIPTDEEIWAE |
| 4. | Aldehyde | MNKDTLIPTTKDLKVKTNGENINLKNYKDNSSCFGVFENVENAISSAV |
| | dehydrogenase from | HAQKILSLHYTKEQREKIITEIRKAALQNKEVLATMILEETHMGRYED |
| | Clostridium | KILKHELVAKYTPGTEDLTTTAWSGDNGLTVVEMSPYGVIGAITPSTN |
| | beijerinckii | PTETVICNSIGMIAAGNAVVENGHPCAKKCVAFAVEMINKAIISCGGP |
| | (Accession No. | ENLVTTIKNPTMESLDAIIKHPSIKLLCGTGGPGMVKTLLNSGKKAIG |
| | Q716S8) | AGAGNPPVIVDDTADIEKAGRSIIEGCSFDNNLPCIAEKEVFVFENVA |
| | | DDLISNMLKNNAVIINEDQVSKLIDLVLQKNNETQEYFINKKWVGKDA |
| | | KLFLDEIDVESPSNVKCIICEVNANHPFVMTELMMPILPIVRVKDIDE |
| | | AIKYAKIAEQNRKHSAYIYSKNIDNLNRFEREIDTTIFVKNAKSFAGV |
| | | GYEAEGFTTFTIAGSTGEGITSARNFTRQRRCVLAG |
| 5. | Aldehyde | MQINDIESAVRKILAEELDNASSNSAAVTNDDGHRGIFTNVDDAIAAA |
| | dehydrogenase from | KAAQEVYRDKPIAVRQQVIDAIKEGFRPYIEKMAKDIKEETGMGTVEA |
| | Limosilactobacillus | KIAKLHNALYNTPGPEILEPVVENGDGGMVMYERLPYGVIGAVGPSTN |
| | reuteri (Accession | PSETVIANAIMMLAGGNTLYFGAHPGAKNITRWTIEKMNDFIADATGL |
| | No. A0AOS4NP03) | HNLVVSIETPTIESVQQMMQHPDIAMLAVTGGPAVVHQAMTSGKKAVG |
| | | AGPGNPPAMVDATADIDLAAHNIIASASFDNDILCTAEKEVVAESSIK |
| | | DELIRKMQDEGAFVVNREQADKLADMCIQDNGTPDRKFVGKDATYILD |
| | | QANIPYTGHPVEIICELPKEHPLVMTEMLMPILPVVSCPTFDDVLKTA |
| | | VEVEQGNHHTATIHSNNLKHINNAAHRMQCSIFVVNGPSYVGTGVADN |
| | | GAHSGASALTIATPTGEGTCTARTFTRRVRLNSPQGFSVRNWY |
| 6. | Aldehyde | MTVNEQLVQDIIKNVVASMQLTQTNKTELGVFDDMNQAIEAAKEAQLV |
| | dehydrogenase from | VKKMSMDQREKIISAIRKKTIEHAETLARMAVEETGMGNVGHKILKHQ |
| | Lachnoclostridium | LVAEKTPGTEDITTTAWSGDRGLTLVEMGPFGVIGAITPCTNPSETII |
| | phytofermentans | CNTIGMLAGGNTVVENPHPAAIKTSNFAVQLINEASLSAGGPVNIACS |
| | (Accession No. | VRKPTLDSSKIMMSHQDIPLIAATGGPGVVTAVLQSGKRGIGAGAGNP |
| | A9KN57) | PVLVDETADIRKAAEDIINGCTFDNNLPCIAEKEVVAIDAIANELMNY |
| | | MVKEQGCYAITKEQQEKLTNLVITPKGLNRNCVGKDARTLLGMIGIDV |
| | | PSNIRCIIFEGEKEHPLISEELMMPILGIVRAKSEDDAVEKAVWLEHG |
| | | NRHSAHIHSKNVDRITTYAKAIDTAILVKNAPSYAAIGFGGEGFCTFT |
| | | IASRTGEGLTSASTFTKRRRCVMSDSLCIR |
| 7. | Aldehyde | MNTSELETLIRTILSEQLTTPAQTPVQPQGKGIFQSVSEAIDAAHQAF |
| | dehydrogenase from | LRYQQCPLKTRSAIISAMRQELTPLLAPLAEESANETGMGNKEDKELK |
| | Salmonella | NKAALDNTPGVEDLTTTALTGDGGMVLFEYSPFGVIGSVAPSTNPTET |
| | typhimurium | IINNSISMLAAGNSIYESPHPGAKKVSLKLISLIEEIAFRCCGIRNLV |
| | (Accession No. | VTVAEPTFEATQQMMAHPRIAVLAITGGPGIVAMGMKSGKKVIGAGAG |
| | Q9XDN1) | NPPCIVDETADLVKAAEDIINGASFDYNLPCIAEKSLIVVESVAERLV |
| | | QQMQTFGALLLSPADTDKLRAVCLPEGQANKKLVGKSPSAMLEAAGIA |
| | | VPAKAPRLLIALVNADDPWVTSEQLMPMLPVVKVSDEDSALALALKVE |
| | | EGLHHTAIMHSQNVSRLNLAARTLQTSIFVKNGPSYAGIGVGGEGFTT |
| | | FTIATPTGEGTTSARTFARSRRCVLTNGESIR |

The sequences in the following TABLE 3 illustrate template nucleotide sequences that can be used to generate the ACL variant sequences and/or compositions and perform the methods described herein. As needed, an RNA sequence can be readily deduced from the DNA sequence. While the below sequences are naturally-occurring, a person of ordinary skill in the art would understand that such sequences may be codon optimized for use in the present disclosure.

TABLE 3
ACL TEMPLATE NUCLEOTIDE SEQUENCES

| SEQ | | |
|---|---|---|
| ID | | |
| NO: | Description | Nucleotide Sequence |
| 8. | YALIOE20405p from | ATGACACAGATTATCCATAACGCCACCATCCCCAATATCCCCGTGGAC |
| | Yarrowia lipolytica | CAGCTCTACGACGGCAAGATCACCGACTTCATTCGAAGCGGAGGCCAC |
| | CLIB122 | TCCAACGAAACCAAGCCTTCGGTGATCGATGCCAAAACAGGCCAGACT |
| | | CTTTCCCAGGCGGAAATGTGGCAGCTGTCGGACAAGTACGCGGCACTT |
| | | CTAAGCTCGCAGTACGGTCTATGCAGACACAGAGACAACGAGCTGGAC |
| | | CCATCCATGGGAGATGTGCTCATCACCTTTTTTGGAAACGTGATCCTC |
| | | GCCCCTGTGGTCCATTGGGCTGCTCTCGACCTCGGAGCAACCATTTCT |
| | | CCCGGATCCACAGGCTACTCGGCCCAGGATCTCGCTCACCAGTTCAGA |
| | | GTCACCACTCCCAAGGTCGTTGTGTATGCCAAGGCGTTCAAGGATGTG |
| | | GTGGACGAGGCTACGAAGCTGTACAACTCGCCCAACCCCCCCGCACTT |
| | | GTTGAGCTCGAGGCGCTGGACAAACAGGCCCGAATGGTTGGAAACCAC |
| | | AAGGTCGAACACACCCGAAAGATCAAGCTGGCCCCCCACGAGTCGCGA |
| | | ACTCGAATCGCGTACCTTGGCATGTCTTCAGGTACCTCCGGAGGAGTT |
| | | TCAAAGGCTGTCAGGCTCACCCATTCCAATCTCACGTCGTGTTCTGAA |
| | | ATCTCTAACAAAGCCTCCGAGTCCCTCGCAACTGACCAGCAGATCGCT |
| | | GCCGCCATTATCCCCGTGTCTCATCTGTTTGGACTGTCGAAATTCCTC |
| | | ATTGGCAACCCCCACGCCGGAGCCACCACTGTCTATCACAATGGGTTC |
| | | GATCTGATCGAGGTGCTGGAGGCACAGAAGAAGTATAAAGTCAACTCG |
| | | TGGACCCTGGTACCCCCCATCATTGTACTGCTCACCAAACACCCCATT |
| | | GTCGAGAAGTACATTCCTTCTCTCAGGGCCCACATGCGAGCCATCCTC |
| | | TCGGGAGCTGCTCCTCTGGGTGCCAATGTCACAGAGGCTCTTTTGACC |
| | | AGAGTCACTGGCAACAAGTTTGGCGAGTCCCCCGAGGGCGGTCTGCGA |
| | | ATCGTCCAGGGCTACGGACTTACAGAGACGTCTCCCGTAGCCACTCTG |
| | | TTTGACCCCGAAGACAAGGAACGACACATTAGGTCGTGTGGAAAGCTC |
| | | GTGCCCAACTCGCAGGTTCGAATTGTGAACGAAGACGGCGTTGATCAG |
| | | CCCGCCTACGATGTGGACCCCAACGAGCTGGACGAGGCCATCAAACAG |
| | | GGCACTCTGCCAGTCGGAGAGCTTTGGATCAGAGGTCCCCAGGTTATG |
| | | GACGGCTACCATAACAACCCCGAGGCCAACGAAGCCTGTTTCGTCAAG |
| | | GCTGACGATGCTGAAGCAGATACTGCCTACTATAACAGACACTGGTTC |
| | | CGAACCGGAGACGTTGCTCTGGTCGACAAGCAGGGCAGATACATGATT |
| | | GTGGACCGAACCAAGGAGATGATCAAGAGTCAGGGTAAGCAGGTTGCT |
| | | CCTGCCGAGCTCGAAGACATGCTCCTGGGACACGCACAGGTGGCAGAT |
| | | ACCGCAGTCATTGGTATTCAGGACGTGGAGAAGGGTAACGAGGCTCCC |
| | | CGAGCTTTTGTTGTGCTCAAGGACCCGAAGTACGACGCTGTGGAGATT |
| | | AAAACATGGCTGGACAAGCAGCTTCCCAAGTACAAGCAGCTTCATGCT |
| | | GGCATCGTGGTGATTGATGCCATTCCCAAGAACGCCAGTGGCAAGATT |
| | | CTGCGACGATTGTTGCGTGCTAGAAAGGACGATGTTGTTCTGGGTCTC |
| | | AACAAGGCCAAGCTGTAA |
| 9. | CoA ligase from | ATGAACCAGATAGATGCCAGCGGCATAGAGCTGCTGCCACATGACGTG |
| | Actibacterium | CATCAGGAAAGCCGCCCGGATGGGTCAATTCTGCTTCGGTCTACGCAT |
| | lipolyticum | TCTCTTGGGGCGGTCGCGCCGAACACTGGCGCATGGTTGCACCGGTGG |
| | | GCGAAAGAGGCGCCGGGGCGCGTTGCCATCGCAGAACGTGCGGGTGCG |
| | | GGCTGGCGGGAACATACCTATAACGAATTGCTAAACATGGTGCGCAAC |
| | | GTTGCTGGCGCGCTTTTGGCGCGGGGCTTGGGCGCGGATAGCCCAATC |
| | | GTGGTCATGTCCGGCAACGGGGTTGATCATGCGATCCTATCGCTTGCC |
| | | GCGCAATATGTTGGTGTACCCATCGTCCCCCTTGCAGAGCAGTATTCG |
| | | CTGATTCCCGAAGCCCACGGGCGGCTGATCTATGTGCTTGAAAAGGTG |
| | | AAGCCCGCGATGGCGTTTGTTTCGGACGCGGCGCAGTATGCCTCTGCG |
| | | CTGTCACTGCCACAGCTTGAAGGGGTGGAACTGGTCGCGGTCAAAACA |
| | | GATGGCGCGCCTGTGCCAGTGACCGCATTTGCCGATTTGCTGGGCCAC |
| | | GGACAGGGGGGTGTCGACGACGCCCACGCCAAAGTCGGGCCAGATACG |
| | | CTGGCTAAGATTTTGTTCACTTCGGGGTCGTCCTCGGACCCAAAGGGG |
| | | GTTCTGACGACGCACCGCATGATGTGTGCCAACCAAACGCAGATCTCT |
| | | GACGCGCTGCGGTTCTTGAAGGCGCGGCCACCCCGTATTCTGGACTGG |
| | | CTGCCATGGAACCATGTGTTCGGCGGAAGCCACAACTTCAACATGATG |
| | | CTCGCCAATGGCGGCAGCCTTTATATCGACAACGGAAAGCCCACCAAA |
| | | GCTGGCTTCGAAGACACCCTTCGCAACATCAAAGAGCATCCGGGAACC |
| | | TTGGCGTTCAACGTGCCCGTGGGCTATGCGATGCTTGTCAACGCGATG |
| | | GAGCGTGACCCGGCCCTGCGACGCGCCTATTTCAAAGAACTTGACCTT |
| | | ATCTTTTACGCGGGCGCGTCCCTGCCGCAGGATGTGTGGGAAAGTCTG |
| | | GAACGTTTCGCGATGGAAGAACGGGGCAGCCTGCCTCTGATGGCGTCC |
| | | AGCTGGGGCATGACCGAAACCGCGCCCGCCACGATTATGGTCCATGAA |
| | | CCTATCGGGCGGTCAGGTGTGATCGGTGTTCCGTTGCCGGGGGCAGAG |
| | | GTCAAGCTGTTGCCCGATGATGAAATGCGCTGTGAATTGCGGGTCAAA |
| | | GGGCCGAACATCATGCGCGGCTATTTCAACGATCCGGCGAAAACCGCC |
| | | GAAGCGTTTGATGACGAGGGGTTCCTGGTAACCGGCGATGCCGTGCGC |
| | | TTTGTCGACGATGCGGATCCATCGCGCGGGTTGGTGTTTGACGGGCGT |
| | | GTCTCTGAAGATTTCAAGTTGTTGACCGGAACATGGGTGCAGGCAGGC |
| | | GTCTTGCGTCTGGATGCGCTTAAGGCGCTTGCCGGTTTGGTGCAGGAC |
| | | GTTGTGATCTGCGGCCATGATCGTGATCAGATTGGCCTGTTCATCTTT |
| | | CCGCTTCCGGGGGCGGTGTCTGACACCTCTGCCGGGGCGGTCATTGAT |
| | | CCGGCGCTTAGCGCCCAGATCGAAGAGCGCCTGCGCGACATGGCCAAA |
| | | ACCGCAACCGGGTCTGCCAAGCGCATTAACCGCGCAATTATCCTGTCA |
| | | GAGCCGCCGCAGTTGAAGGACCACGAGATCACCGACAAGGGCAGCCTG |
| | | AACATCCGCAAAATCCTGACACGCCGCGCCGATCTGTTGGAACGGCTT |
| | | TATGACAACGAAGACCCGGCCCTGATCCGGGTTTGA |

The sequences in the following TABLE 4 illustrate template nucleotide sequences that can be used to generate the ALDH variant sequences and/or compositions and perform the methods described herein. As needed, an RNA sequence can be readily deduced from the DNA sequence.

TABLE 4
ALDH TEMPLATE NUCLEOTIDE SEQUENCES

| SEQ | | |
|---|---|---|
| ID | | |
| NO: | Description | Nucleotide Sequence |
| 10. | Codon optimized Aldehyde | ATGGAACAGGCCGTTAAAGATTATCTGGATAAAATGGTTGC |
| | dehydrogenase from | AGCAAGCCGTATTGCACAGCAAGAATTTGCAACCTATCCGC |
| | Acidaminococcus | AAGAAACCGTTGATAAAGCAGTTCGTACCGTGGGTAAAGCC |
| | massiliensis | ATTTATGATAATGCAGAACTGCTGGCACATATGGCAGTTGA |
| | | TGAAACCAAAATGGGTAACTATGCCGACAAAATTGCCAAAT |
| | | GCGTGAACAAAAGCAAAAGCGTTTGGTGGCGTATGAAAGAC |
| | | AAAAAAAGCCGTGGTATTATCAAACGCATTCCGGAACTGGG |
| | | TTTAGTTGAAGTTGCAAAACCGATTGGTGTTATTGGTTGTG |
| | | TTGCACCGACCACCAATCCGGTTATTAATGTTATGCAGAAT |
| | | GCAATGTGTGCCCTGAAATGTGGTAATAGCATGATTGTTAG |
| | | TCCGCATCCGCGTGCAAAACATTCAAGCGTTAAAACCGTGG |
| | | AAGTGATTAATGAAGCACTGGCAGCACTGGGCATGCCGAAA |
| | | AATCTGATTCAGGTTATTACCGAACCGAGCATGGAACTGAG |
| | | CGCAGGTCTGATGAGCGCAGTTGATCTGTGTATTTGTACCG |
| | | GTGGTCCTGGTCTGGTTAAAGCAGCATATAGCAGCGGTAAA |
| | | CCGGCAATTGGTGTTGGTCAGGGTAATGTTCAGGTTCTGGT |
| | | TGATCGTGATGCCGATCTGGATCAGGTTGCAGCCATGGTTA |
| | | TTAAAGGTCGCACATTTGATAATGGTGTTCTGTGTACCTGT |
| | | GAACAGAATGTTATTTGTCCCGAGGATAAAAAAGAAGAGAT |
| | | GATTGCAGCCCTGAAAAAACATGGTGCCTATTATATCGGTA |
| | | ATAGCGAAGATGCAGCAAAACTGCGTGATACCGCATTTCCG |
| | | AATGGTGGTCCGGTTAGCAAAGAATATCCGGGTGCCAGCGT |
| | | GAAAAAAATCGCACAGCTGAGCGGTATTCAGGGTATTCCGG |
| | | AAGATGCAAAAGTTATTGTTAGCTGTACCCGTGGTTATGGT |
| | | AAAGATGAACCGCTGGCAAAAGAAAAACTGTTTCCGGTTCT |
| | | GGCCTTTTTCACCTATGATAAATGGGAAGATGCCATCCATA |
| | | TTGCCAAAACCAATCTGGAAATGGAAGGTATTGGTCATAGC |
| | | GTTGTGATTCATAGCAATACACCGGAACATATTGAAGCAGT |
| | | TGCAGAAGCAATTCCGGTTAGTCGTTTTGCAGTTAATCAGG |
| | | TTGGTGGCACCAATTTAGGTGGTGCAATGGATAATGGTCTG |
| | | AATCCGACCACAACCTTAGGTTGCGGCACCTGGGGTAATAA |
| | | TAGCATTAGCGAAAACTTCACCTACTATCATCTGATGAATC |
| | | TGACCCGTGTTAGCTATCGTGTTCCGGATATGTATATTCCG |
| | | ACCGATGAAGAAATTTGGGCAGAATAA |
| 11. | Codon optimized aldehyde | ATGAATAAAGACACACTAATACCTACAACTAAAGATTTAAA |
| | dehydrogenase from | AGTAAAAACAAATGGTGAAAACATTAATTTAAAGAACTACA |
| | Clostridium beijerinckii | AGGATAATTCTTCATGTTTCGGAGTATTCGAAAATGTTGAA |
| | | AATGCTATAAGCAGCGCTGTACACGCACAAAAGATATTATC |
| | | CCTTCATTATACAAAAGAGCAAAGAGAAAAAATCATAACTG |
| | | AGATAAGAAAGGCCGCATTACAAAATAAAGAGGTCTTGGCT |
| | | ACAATGATTCTAGAAGAAACACATATGGGAAGATATGAGGA |
| | | TAAAATATTAAAACATGAATTGGTAGCTAAATATACTCCTG |
| | | GTACAGAAGATTTAACTACTACTGCTTGGTCAGGTGATAAT |
| | | GGTCTTACAGTTGTAGAAATGTCTCCATATGGTGTTATAGG |
| | | TGCAATAACTCCTTCTACGAATCCAACTGAAACTGTAATAT |
| | | GTAATAGCATAGGCATGATAGCTGCTGGAAATGCTGTAGTA |
| | | TTTAACGGACACCCATGCGCTAAAAAATGTGTTGCCTTTGC |
| | | TGTTGAAATGATAAATAAGGCAATTATTTCATGTGGCGGTC |
| | | CTGAAAATCTAGTAACAACTATAAAAAATCCAACTATGGAG |
| | | TCTCTAGATGCAATTATTAAGCATCCTTCAATAAAACTTCT |
| | | TTGCGGAACTGGGGGTCCAGGAATGGTAAAAACCCTCTTAA |
| | | ATTCTGGTAAGAAAGCTATAGGTGCTGGTGCTGGAAATCCA |
| | | CCAGTTATTGTAGATGATACTGCTGATATAGAAAAGGCTGG |
| | | TAGGAGCATCATTGAAGGCTGTTCTTTTGATAATAATTTAC |
| | | CTTGTATTGCAGAAAAAGAAGTATTTGTTTTTGAGAATGTT |
| | | GCAGATGATTTAATATCTAACATGCTAAAAAATAATGCTGT |
| | | AATTATAAATGAAGATCAAGTATCAAAATTAATAGATTTAG |
| | | TATTACAAAAAAATAATGAAACTCAAGAATACTTTATAAAC |
| | | AAAAAATGGGTAGGAAAAGATGCAAAATTATTCTTAGATGA |
| | | AATAGATGTTGAGTCTCCTTCAAATGTTAAATGCATAATCT |
| | | GCGAAGTAAATGCAAATCATCCATTTGTTATGACAGAACTC |
| | | ATGATGCCAATATTGCCAATTGTAAGAGTTAAAGATATAGA |
| | | TGAAGCTATTAAATATGCAAAGATAGCAGAACAAAATAGAA |
| | | AACATAGTGCCTATATTTATTCTAAAAATATAGACAACCTA |
| | | AATAGATTTGAAAGAGAAATAGATACTACTATTTTTGTAAA |
| | | GAATGCTAAATCTTTTGCTGGTGTTGGTTATGAAGCAGAAG |
| | | GATTTACAACTTTCACTATTGCTGGATCTACTGGTGAGGGA |
| | | ATAACCTCTGCAAGGAATTTTACAAGACAAAGAAGATGTGT |
| | | ACTTGCCGGCTAA |
| 12. | Codon optimized aldehyde | ATGCAGATTAACGATATTGAAAGTGCTGTTCGCAAGATCCT |
| | dehydrogenase from | AGCAGAAGAATTAGATAATGCTAGCTCTAACAGTGCGGCAG |
| | Limosilactobacillus | TTACTAATGATGATGGACATCGTGGTATTTTTACTAATGTT |
| | reuteri | GATGACGCAATTGCTGCTGCTAAAGCTGCCCAAGAAGTATA |
| | | TCGTGACAAGCCAATTGCAGTGCGGCAACAGGTGATTGATG |
| | | CTATCAAGGAAGGTTTCCGTCCTTACATTGAAAAGATGGCA |
| | | AAGGACATTAAAGAAGAAACCGGAATGGGAACAGTCGAAGC |
| | | AAAGATTGCTAAGTTGCACAATGCTTTGTACAACACTCCTG |
| | | GTCCAGAAATTCTTGAACCGGTTGTCGAAAATGGTGATGGC |
| | | GGAATGGTTATGTATGAACGGTTGCCATACGGTGTTATCGG |
| | | TGCTGTTGGTCCAAGTACTAACCCATCCGAAACAGTTATTG |
| | | CCAATGCAATTATGATGCTTGCAGGTGGAAATACCCTTTAC |
| | | TTTGGTGCTCACCCTGGTGCTAAGAATATCACTCGTTGGAC |
| | | GATTGAAAAGATGAATGATTTCATTGCTGATGCAACTGGTC |
| | | TTCACAATTTAGTTGTAAGCATTGAAACACCAACAATTGAA |
| | | TCTGTTCAACAAATGATGCAACATCCTGATATTGCTATGCT |
| | | TGCCGTAACCGGTGGACCTGCTGTTGTTCACCAAGCAATGA |
| | | CTAGCGGTAAGAAGGCTGTTGGTGCTGGTCCTGGTAATCCT |
| | | CCTGCAATGGTTGATGCTACCGCTGATATTGACTTAGCTGC |
| | | TCATAATATTATTGCTTCAGCTTCATTCGATAATGACATTT |
| | | TATGTACTGCTGAAAAGGAAGTTGTTGCCGAAAGCAGTATT |
| | | AAGGATGAATTAATCCGCAAGATGCAAGATGAAGGTGCATT |
| | | TGTTGTTAACCGTGAACAGGCTGACAAACTCGCCGATATGT |
| | | GCATCCAAGACAACGGTACTCCTGATCGGAAGTTTGTTGGA |
| | | AAGGATGCAACCTACATCTTAGATCAAGCTAATATTCCATA |
| | | TACTGGTCACCCGGTAGAAATTATTTGTGAATTGCCAAAGG |
| | | AACACCCATTGGTAATGACTGAAATGTTGATGCCAATCTTA |
| | | CCAGTTGTTTCTTGCCCAACATTTGATGATGTTTTGAAGAC |
| | | TGCTGTTGAAGTAGAACAAGGCAACCACCATACTGCAACTA |
| | | TTCACTCCAACAACTTAAAGCATATTAATAATGCTGCTCAC |
| | | CGGATGCAATGTTCAATCTTCGTTGTTAATGGTCCATCCTA |
| | | TGTTGGAACAGGTGTTGCTGATAATGGTGCTCACTCAGGTG |
| | | CTTCAGCATTAACAATTGCTACGCCAACCGGTGAAGGAACT |
| | | TGTACTGCAAGAACCTTTACGCGTCGTGTTCGTTTGAATTC |
| | | GCCACAAGGGTTCTCAGTTCGTAATTGGTACTAA |
| 13. | Codon optimized aldehyde | GTGACAGTGAATGAACAATTGGTTCAAGACATCATTAAAAA |
| | dehydrogenase from | TGTCGTAGCGAGTATGCAATTGACACAAACAAATAAGACAG |
| | Lachnoclostridium | AACTTGGCGTATTCGACGACATGAATCAGGCAATTGAAGCT |
| | phytofermentans | GCAAAAGAAGCTCAATTGGTTGTAAAGAAGATGTCGATGGA |
| | | TCAGAGAGAGAAAATCATCTCTGCGATACGTAAGAAAACGA |
| | | TTGAACATGCAGAAACACTTGCCCGTATGGCGGTAGAAGAA |
| | | ACTGGCATGGGTAATGTTGGACATAAGATTTTAAAACACCA |
| | | GCTGGTAGCAGAAAAGACACCTGGAACAGAAGATATCACGA |
| | | CTACTGCTTGGTCTGGTGATCGTGGTTTAACCTTAGTTGAG |
| | | ATGGGACCATTTGGTGTGATTGGTGCAATAACACCATGTAC |
| | | TAACCCAAGTGAAACAATCATTTGTAACACGATTGGCATGT |
| | | TAGCTGGTGGAAACACAGTGGTATTTAATCCGCATCCAGCA |
| | | GCGATAAAGACCTCGAACTTTGCTGTACAGTTAATTAATGA |
| | | AGCATCACTTTCGGCTGGAGGTCCAGTCAACATTGCTTGTT |
| | | CTGTGAGAAAACCTACCCTAGATAGCAGTAAGATAATGATG |
| | | TCTCATCAGGACATTCCATTGATTGCTGCTACTGGAGGTCC |
| | | AGGCGTAGTTACTGCTGTATTACAAAGCGGAAAACGAGGAA |
| | | TCGGTGCAGGAGCAGGAAATCCACCGGTTTTAGTAGATGAA |
| | | ACAGCTGATATTAGAAAAGCAGCCGAAGATATCATTAATGG |
| | | ATGTACCTTTGATAATAATCTTCCATGTATTGCAGAAAAAG |
| | | AAGTTGTTGCAATTGATGCTATAGCAAATGAATTAATGAAC |
| | | TACATGGTAAAAGAACAAGGTTGTTATGCAATCACCAAAGA |
| | | ACAGCAAGAAAAGTTAACTAATCTTGTGATAACACCCAAAG |
| | | GATTAAATCGTAATTGCGTTGGTAAAGATGCCAGAACATTA |
| | | CTTGGTATGATTGGAATTGATGTTCCATCCAATATTCGTTG |
| | | TATCATTTTTGAAGGTGAGAAAGAACATCCTTTGATTTCTG |
| | | AAGAGTTAATGATGCCGATTCTTGGAATTGTTCGAGCTAAG |
| | | AGCTTCGATGATGCGGTAGAAAAAGCGGTTTGGTTAGAACA |
| | | TGGTAATCGACATTCCGCACATATTCATTCCAAGAATGTGG |
| | | ACCGTATCACAACTTATGCAAAAGCGATTGATACAGCTATC |
| | | CTTGTTAAAAATGCACCTTCCTATGCAGCAATCGGATTTGG |
| | | TGGTGAAGGATTTTGTACCTTTACTATAGCAAGTAGAACCG |
| | | GTGAAGGCTTGACGAGTGCAAGTACATTTACAAAACGCAGA |
| | | CGATGCGTGATGTCGGATAGTTTGTGTATCCGATAA |
| 14. | Codon optimized aldehyde | ATGAATACTTCTGAACTCGAAACCCTGATTCGCACCATTCT |
| | dehydrogenase from | TAGCGAGCAATTAACCACGCCGGCGCAAACGCCGGTCCAGC |
| | Salmonella typhimurium | CTCAGGGCAAAGGGATTTTCCAGTCCGTGAGCGAGGCCATC |
| | | GACGCCGCGCACCAGGCGTTCTTACGTTATCAGCAGTGCCC |
| | | GCTAAAAACCCGCAGCGCCATTATCAGCGCGATGCGTCAGG |
| | | AGCTGACGCCGCTGCTGGCGCCCCTGGCGGAAGAGAGCGCC |
| | | AATGAAACGGGGATGGGCAACAAAGAAGATAAATTTCTCAA |
| | | AAACAAGGCTGCGCTGGACAACACGCCGGGCGTAGAAGATC |
| | | TCACCACCACCGCGCTGACCGGCGACGGCGGCATGGTGCTG |
| | | TTTGAATACTCACCGTTTGGCGTTATCGGTTCGGTCGCCCC |
| | | AAGCACCAACCCGACGGAAACCATCATCAACAACAGTATCA |
| | | GCATGCTGGCGGCGGGCAACAGTATCTACTTTAGCCCGCAT |
| | | CCGGGAGCGAAAAAGGTCTCTCTGAAGCTGATTAGCCTGAT |
| | | TGAAGAGATTGCCTTCCGCTGCTGCGGCATCCGCAATCTGG |
| | | TGGTGACCGTGGCGGAACCCACCTTCGAAGCGACCCAGCAG |
| | | ATGATGGCCCACCCGCGAATCGCAGTACTGGCCATTACCGG |
| | | CGGCCCGGGCATTGTGGCAATGGGCATGAAGAGCGGTAAGA |
| | | AGGTGATTGGCGCTGGCGCGGGTAACCCGCCCTGCATCGTT |
| | | GATGAAACGGCGGACCTGGTGAAAGCGGCGGAAGATATCAT |
| | | CAACGGCGCGTCATTCGATTACAACCTGCCCTGCATTGCCG |
| | | AGAAGAGCCTGATCGTAGTGGAGAGTGTCGCCGAACGTCTG |
| | | GTGCAGCAAATGCAAACCTTCGGCGCGCTGCTGTTAAGCCC |
| | | TGCCGATACCGACAAACTCCGCGCCGTCTGCCTGCCTGAAG |
| | | GCCAGGCGAATAAAAAACTGGTCGGCAAGAGCCCATCGGCC |
| | | ATGCTGGAAGCCGCCGGGATCGCTGTCCCTGCAAAAGCGCC |
| | | GCGTCTGCTGATTGCGCTGGTTAACGCTGACGATCCGTGGG |
| | | TCACCAGCGAACAGTTGATGCCGATGCTGCCAGTGGTAAAA |
| | | GTCAGCGATTTCGATAGCGCGCTGGCGCTGGCCCTGAAGGT |
| | | TGAAGAGGGGCTGCATCATACCGCCATTATGCACTCGCAGA |
| | | ACGTGTCACGCCTGAACCTCGCGGCCCGCACGCTGCAAACC |
| | | TCGATATTCGTCAAAAACGGCCCCTCTTATGCCGGGATCGG |
| | | CGTCGGCGGCGAAGGCTTTACCACCTTCACTATCGCCACAC |
| | | CAACCGGTGAAGGGACCACGTCAGCGCGTACTTTTGCCCGT |
| | | TCCCGGCGCTGCGTACTGACCAACGGCTTTTCTATTCGCTA |
| | | A |

It is understood that modifications which do not substantially affect the activity of the various embodiments of this invention are also provided within the definition of the invention provided herein. Accordingly, the following examples are intended to illustrate but not limit the present invention.

### EXAMPLES

### Example 1. Metagenomic and Protein Engineering of the ACL Template Library

### 1.1. ACL Seed Enzymes

Potential ACL seed enzymes were tested to find the best performing seed which was then used to generate a portion of the ACL Template Library. Enzymes were synthesized and expressed in an IPTG inducible plasmid in E. coli (ΔygbC, ΔtesB, ΔyciA) and evaluated in a cell lysate assay for substrate consumption. Activity was determined as change in absorbance (ΔA₄₁₂) relative to a lysate that contained no ACL (negative control), where a reduction in A₄₁₂ indicates consumption of CoA substrate.

Of the enzymes that showed signal decrease compared to the negative control (no cell lysate), liquid chromatography-tandem mass spectrometry (LCMS) confirmed detection of HHA-CoA as a reaction product of the enzyme exhibiting the lowest A₄₁₂ value (SEQ ID NO: 1).

### 1.2. Screening and Sourcing of Metagenomic Library ACL Template Enzyme Candidates

Genes encoding ACL candidate template enzymes were identified bioinformatically using the amino acid sequence identified in Example 1.1. Gene candidates were synthesized, expressed and evaluated in an in vitro cell lysate assay as described above. Three of the candidates demonstrated good activity, and the best hit was selected as a template candidate (SEQ ID NO: 2) (TABLE 5).

TABLE 5
Metagenomic ACL Template Candidates

| Accession | | Average | |
|---|---|---|---|
| No. | Organism | rate | Std |
| A0A238JIY9 | Confluentimicrobium | 0.3735 | 0.010606602 |
| (SEQ ID | Epolyticum | | |
| NO: 2) | | | |

### 1.3. ACL Template Library

In total, two enzymes (homolog 1—SEQ ID NO: 1 and homolog 2—SEQ ID NO: 2), as set forth in TABLE 1 were selected as template candidates.

### Example 2. Assaying the ACL Template Library

### 2.1. In Vitro Cell Lysate Assay for Substrate Specificity

Based on the results from Example 1, the genes encoding the ACL enzyme candidates of TABLE 6 were synthesized, expressed and evaluated for substrate specificity in an in vitro cell lysate assay as described in Example Example 1.

Here, ACL activity on HHA, acetate, propionate, 4-HB, adipate, and succinate substrates were evaluated. Activity was determined as change in absorbance (Δ A₄₁₂) relative to a lysate that contained no ACL (negative control), where a reduction in A₄₁₂ indicates consumption of the substrate. While activity on HHA is preferred and activity on succinate or adipate is acceptable, activity on 4-HB is to be avoided. TABLE 6 shows HHA activity for homolog 2 (SEQ ID NO: 2) with considerable off target activity on 4-HB, which was the highest activity in the assay (full data not shown). Homolog 1 (SEQ ID NO: 1) also showed good activity on HHA, with improved specificity.

TABLE 6
ACL Template Candidates in vitro cell lysate assay for substrate specificity

| | SEQ | % | % | Δ A412 relative to control | | | | | | |
|---|---|---|---|---|---|---|---|---|---|---|
| Homo | ID | Identity to | Identity to | HHA | HHA | Acetate | Propionate | 4-HB | Adipate | Succinate |
| log # | NO: | homolog 4 | homolog 5 | (10 mM) | (2 mM) | (10 mM) | (10 mM) | (10 mM) | (10 mM) | (10 mM) |
| 1 | 1 | — | 14.72 | 0.401 | 0.099 | −0.010 | −0.003 | 0.003 | 0.002 | −0.029 |
| 2 | 2 | 14.72 | — | 0.488 | 0.122 | 0.120 | 0.222 | 0.096 | 0.031 | −0.024 |

### 2.2. Kinetic Characterization

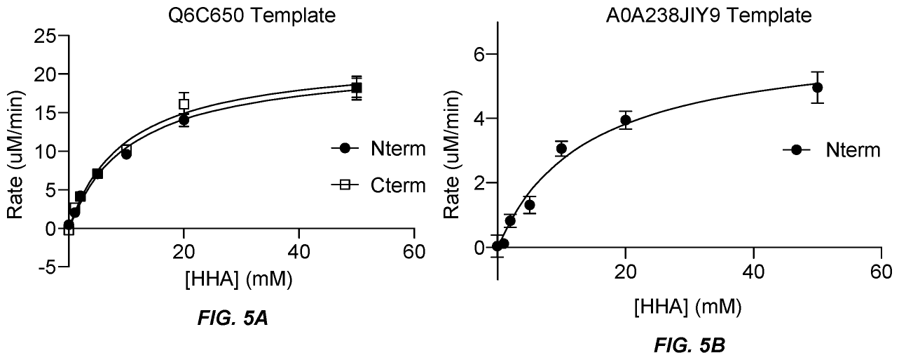

In vitro biochemical characterization was performed on the ACL template enzymes identified in Example 2.1. FIGS. 5A and 5B shows the Michaelis-Menten saturation curve obtained from reactions with HHA at room temperature. For homolog 1 (SEQ ID NO: 1) N- and C-terminal purification tags were used (FIG. 5A), for homolog 2 (SEQ ID NO: 2), only the N-terminal tag yielded sufficient active protein after purification (FIG. 5B). Calculated kinetic parameters are listed in TABLE 7.

TABLE 7
ACL Michaelis-Menten Kinetic Parameters*

| | Homolog 1 | Homolog 1 | Homolog 2 |
|---|---|---|---|
| | (N-tag) | (C-tag) | (N-tag) |
| Vmax (uM/min | 21.9 | 22.3 | 6.5 |
| | (19.3 to 25.1) | (19.2 to 26.4) | (5.2 to 8.4) |
| Km (mM) | 11.1 | 10.0 | 14.0 |
| | (7.8 to 15.8) | (6.5 to 15.4) | (8.1 to 25.0) |
| kcat (s⁻¹) | 7.3 | 7.4 | 0.46 |

*95% confidence intervals based on profile likelihood indicated in parenthesis

### Example 3. Engineering and Assaying the ACL Variant Library

An ACL variant library comprising about 2000 proteins was designed based on homolog 1 (SEQ ID NO: 1) and homolog 2 (SEQ ID NO. 2). Approximately 1000 proteins based on homolog 1 (SEQ ID NO: 1) and about 1000 proteins based on homolog 2 (SEQ ID NO: 2), were included in the library.

### 3.1. Primary Screen

In order to evaluate the impact of each selected ACLs variant identified by the methods described in Example 1. Acyl-CoA ligases were expressed from an IPTG inducible plasmid in E. coli (ΔygbC, ΔtesB, ΔyciA) and tested in a cell lysate assay. The resulting strains were evaluated for enzymatic activity in three replicate batches as described using similar processes as described in Example 1.1.

TABLE 8 shows that ACL variants of homolog 1 nos. 336, 942, 645, 803, 783, 840, 835, 429, 968, 663, 871, 862, 160, 303, 259, 951, 686, 272, 87, 877, 858, 884, 118, 140, 96, 954, 42, 197, 183, 543, 866, 433, 436, 369, 232, 927, 324, 851, 540, 905, 113, 950, 745, 771, 952, 772, 274, 80, 562, 34, 46, 797, 391, 653, 164, 555, 901, 812, 79, 980, 150, and 553 have the highest activity level relative to homolog 1 and the highest activity levels on HHA.

TABLE 8
Exemplary Engineered ACLs Using Homolog 1 (SEQ ID NO: 1) as the Template
with Positional Alteration(s) and Corresponding Activity on HHA

| Variant | | Activity relative | Active |
|---|---|---|---|
| # | Mutations to homolog 1* | to homolog 1 | HHA |
| | Homolog 1 | 1 | + |
| 1 | V208F | 0.655 | − |
| 2 | A234F | 1.137 | + |
| 3 | M503L | 0.563 | − |
| 4 | V99M | 0.422 | − |
| 5 | V521E | 1.508 | + |
| 6 | I562V | 0.048 | − |
| 7 | G114A | 0.495 | − |
| 8 | A52R | 1.001 | + |
| 9 | N7T A8S Y19Q G30D S39A V40L K44S Q47R L49Y M54L | −0.079 | − |
| | K60R L64A Y69L C72T R73G E78V S82N M83K L87VAV d88- | | |
| | 89 V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140E V144T T149R | | |
| | Y152I A159G E162SIDAF (“SIDAF” disclosed as SEQ ID NO: | | |
| | 23) d163-166 A170G H189G R213M T220M S221A | | |
| | N227GKWSQD (“GKWSQD” disclosed as SEQ ID NO: 28) | | |
| | d228-232 Q238DISAC (“DISAC” disclosed as SEQ ID NO: 34) | | |
| | d239-242 F255ILLANV (“ILLANV” disclosed as SEQ ID NO: | | |
| | 35) d256-260 Y268IMPR (“IMPR” disclosed as SEQ ID NO: 42) | | |
| | d269-271 I275EEMLQAIQ (“EEMLQAIQ” disclosed as SEQ ID | | |
| | NO: 46) d276-282 N287TVFFA (“TVFFA” disclosed as SEQ ID | | |
| | NO: 51) d288-291 d295 L298IM T300A I304E I309L P310L | | |
| | A314SHVRSV (“SHVRSV” disclosed as SEQ ID NO: 56) d315- | | |
| | 319 A328RELE (“RELE” disclosed as SEQ ID NO: 58) d329-331 | | |
| | L335R T336K T339S N341T L351V K374P E375P I378P P386A | | |
| | Q389E R391K N394D V398N D411TENAPD (“TENAPD” | | |
| | disclosed as SEQ ID NO: 61) d412-416 L419G D433K H436W | | |
| | E443RASFA (“RASFA” disclosed as SEQ ID NO: 66) d444-447 | | |
| | D455G Y459D R461G L471R K474EDGH (“EDGH” disclosed as | | |
| | SEQ ID NO: 67) d475-477 A508P Q509D T513A V521P D540T | | |
| | V542E T546K Q551R I562V I565V | | |
| 10 | L384I | 0.016 | − |
| 11 | A104V | 0.11 | − |
| 12 | K254E | 0.058 | − |
| 13 | R461D | 0.108 | − |
| 14 | L410N | 0.271 | − |
| 15 | T546A | 0.466 | − |
| 16 | K374D | 1.592 | + |
| 17 | L471Y | 0.701 | − |
| 18 | H261Y | 0.979 | − |
| 19 | S226Y | 0.035 | − |
| 20 | S230R | 0.521 | − |
| 21 | L198Y | −0.066 | − |
| 22 | W289M | 0.085 | − |
| 23 | P386S | 0.232 | − |
| 24 | G30D | −0.044 | − |
| 25 | T235H | 0.921 | − |
| 26 | E162S | 0.566 | − |
| 27 | C381V | 0.658 | − |
| 28 | L320F | 1.169 | + |
| 29 | N7T A8S D20W G30D S39A V40L K44T Q47RTYTQKEL | 0.016 | − |
| | (“RTYTQKEL” disclosed as SEQ ID NO: 15) d48-54 K60R L64A | | |
| | Y69L R73G S82N M83K L87VAV d88-89 V94TIEAA (“TIEAA” | | |
| | disclosed as SEQ ID NO: 19) d95-98 I111VTPAN (“VTPAN” | | |
| | disclosed as SEQ ID NO: 20) d112-115 S119T Q121R V129I | | |
| | T131G A138S A140E V144T T149R Y1521 A159G E162RIESF | | |
| | (“RIESF” disclosed as SEQ ID NO: 24) d163-166 A170G H189G | | |
| | R213M S221A E224H N227G E231QDLQ (“QDLQ” disclosed as | | |
| | SEQ ID NO: 33) d232-234 Q238DISAC (“DISAC” disclosed as | | |
| | SEQ ID NO: 34) d239-242 F255ILLANVHY (“ILLANVHY” | | |
| | disclosed as SEQ ID NO: 36) d256-262 Y268IMNR (“IMNR” | | |
| | disclosed as SEQ ID NO: 43) d269-271 I275EEMLQAIQ | | |
| | (“EEMLQAIQ” disclosed as SEQ ID NO: 46) d276-282 | | |
| | N287TSFFA (“TSFFA” disclosed as SEQ ID NO: 52) d288-291 | | |
| | d295 L298IM T300A I304M E306D I309L P310L A314SHVRTV | | |
| | (“SHVRTV” disclosed as SEQ ID NO: 57) d315-319 N329ELE | | |
| | d330-331 L335R T336K T339S N341T E348D L351V K374T | | |
| | E375P I378P P386A Q389E R391K N394D V398N A402L | | |
| | D411TQNAPR (“TQNAPR” disclosed as SEQ ID NO: 62) d412- | | |
| | 416 L419G D433K H436W E443RASFA (“RASFA” disclosed as | | |
| | SEQ ID NO: 66) d444-447 D455G Y459D R461G L471K | | |
| | K474EDGY (“EDGY” disclosed as SEQ ID NO: 68) d475-477 | | |
| | A508P Q509D T513A V521P D540T V542E T546A Q551R d562 | | |
| | I565F D566VE | | |
| 30 | D16H | 0.749 | − |
| 31 | Q281V | 1.028 | + |
| 32 | T290A | 1.256 | + |
| 33 | G251A | 0.362 | − |
| 34 | T484L | 2.202 | ++ |
| 35 | G524S | 0.361 | − |
| 36 | T265R | 0.045 | − |
| 37 | I10V | 0.867 | − |
| 38 | N7T A8S Y19F G30D T36H S39A V40L K44H Q47RTYTQREL | −0.12 | − |
| | (“RTYTQREL” disclosed as SEQ ID NO: 16) d48-54 K60R L64A | | |
| | Y69L R73G S82A M83K L87VAV d88-89 V94TIEAA (“TIEAA” | | |
| | disclosed as SEQ ID NO: 19) d95-98 I111VTPAN (“VTPAN” | | |
| | disclosed as SEQ ID NO: 20) d112-115 S119T Q121R V129I | | |
| | T131G A138S A140E V144T T149R Y1521 A159G E162HFDSF | | |
| | (“HFDSF” disclosed as SEQ ID NO: 25) d163-166 A170G H189G | | |
| | R213M S221A N227G E231Q A234Q Q238D A240S | | |
| | F255ILLANVHY (“ILLANVHY” disclosed as SEQ ID NO: 36) | | |
| | d256-262 Y268IMNR (“IMNR” disclosed as SEQ ID NO: 43) | | |
| | d269-271 I275EEMLRAIQ (“EEMLRAIQ” disclosed as SEQ ID | | |
| | NO: 47) d276-282 N287TFFFA (“TFFFA” disclosed as SEQ ID | | |
| | NO: 53) d288-291 d295 L298IM T300A I304E I309L P310L | | |
| | A314S I319V A328RELE (“RELE” disclosed as SEQ ID NO: 58) | | |
| | d329-331 T336K T339S N341T L351V K374T E375P I378P | | |
| | P386A Q389E R391K N394D V398N A402L L410NTENAPR | | |
| | (“NTENAPR” disclosed as SEQ ID NO: 59) d411-416 L419G | | |
| | D433K H436W E443RASFA (“RASFA” disclosed as SEQ ID | | |
| | NO: 66) d444-447 D455G Y459D R461G L471K K474EDGY | | |
| | (“EDGY” disclosed as SEQ ID NO: 68) d475-477 A508P Q509D | | |
| | T513A V521P D540T V542D T546A Q551R d562 I565F | | |
| | D566VE | | |
| 39 | E306D | 1.787 | + |
| 40 | A496F | 0.925 | − |
| 41 | N227G | 0.726 | − |
| 42 | S223A | 2.9 | ++ |
| 43 | K60R | 0.88 | − |
| 44 | A402V | 0.246 | − |
| 45 | Q51W | −0.023 | − |
| 46 | T235S | 2.196 | ++ |
| 47 | N7T A8S Y19F G30D S39A V40L K44H Q47R L49Y M54L | −0.285 | − |
| | K60R L64A Y69L R73G S82A M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140E V144T T149R | | |
| | Y152I A159G E162HFDAF (“HFDAF” disclosed as SEQ ID NO: | | |
| | 26) d163-166 A170G H189G R213M S221A N227GKWSQSY | | |
| | (“GKWSQSY” disclosed as SEQ ID NO: 29) d228-233 Q238D | | |
| | A240S F255ILLLNVHY (“ILLLNVHY” disclosed as SEQ ID | | |
| | NO: 37) d256-262 Y268IMNR (“IMNR” disclosed as SEQ ID | | |
| | NO: 43) d269-271 L274PEEMLRAIQ (“PEEMLRAIQ” disclosed | | |
| | as SEQ ID NO: 44) d275-282 N287TVFFA (“TVFFA” disclosed | | |
| | as SEQ ID NO: 51) d288-291 d295 L298IM T300A I304E I309L | | |
| | P310L A314SHVRTV (“SHVRTV” disclosed as SEQ ID NO: 57) | | |
| | d315-319 A328RELE (“RELE” disclosed as SEQ ID NO: 58) | | |
| | d329-331 L335R T336K T339S N341T L351V K374P E375P | | |
| | I378P Q389E N394D V398N A402L L410NTENAPD | | |
| | (“NTENAPD” disclosed as SEQ ID NO: 60) d411-416 L419G | | |
| | D433K H436W E443RASFA (“RASFA” disclosed as SEQ ID | | |
| | NO: 66) d444-447 D455G Y459D R461G L471R K474EDGH | | |
| | (“EDGH” disclosed as SEQ ID NO: 67) d475-477 A508P Q509D | | |
| | T513A V521P D540T V542E T546K Q551R d562 I565F | | |
| | D566VE | | |
| 48 | R317H | 0.078 | − |
| 49 | N7T A8S Y19Q D20W G30D S39A V40L K44H | 0.045 | − |
| | Q47RTYTQKEL (“RTYTQKEL” disclosed as SEQ ID NO: 15) | | |
| | d48-54 K60R L64A Y69L C72T R73G P81PG d83 L87VAV d88- | | |
| | 89 V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140E V144T T149R | | |
| | Y152C A159G E162HFDSF (“HFDSF” disclosed as SEQ ID NO: | | |
| | 25) d163-166 A170G H189G R213M T220M S221A N227G | | |
| | E231Q S232N Q238D A240S F255ILLANVHY (“ILLANVHY” | | |
| | disclosed as SEQ ID NO: 36) d256-262 Y268IMNR (“IMNR” | | |
| | disclosed as SEQ ID NO: 43) d269-271 I275KEML QAIQ | | |
| | (“KEMLQAIQ” disclosed as SEQ ID NO: 48) d276-282 | | |
| | N287TVFFA (“TVFFA” disclosed as SEQ ID NO: 51) d288-291 | | |
| | d295 L298IM T300A I304E I309L P310L A314SHVRSV | | |
| | (“SHVRSV” disclosed as SEQ ID NO: 56) d315-319 A328RELE | | |
| | (“RELE” disclosed as SEQ ID NO: 58) d329-331 T336K T339S | | |
| | N341H E348H L351V K374T E375P I378P P386A Q389E | | |
| | R391K N394D V398N A402I L410N A413NNPN (“NNPN” | | |
| | disclosed as SEQ ID NO: 65) d414-416 L419G D433K H436W | | |
| | E443RASFA (“RASFA” disclosed as SEQ ID NO: 66) d444-447 | | |
| | D455G Y459D R461G L471R K474EDGH (“EDGH” disclosed as | | |
| | SEQ ID NO: 67) d475-477 A508P Q509D T513A V521P D540T | | |
| | V542E T546K Q551R d562 I565FV | | |
| 50 | F255M | 0.039 | − |
| 51 | S388T | 1.839 | + |
| 52 | E412Q | 0.618 | − |
| 53 | S247F | 0.862 | − |
| 54 | V40C | 0.726 | − |
| 55 | L198V | 0.182 | − |
| 56 | P98A | 0.798 | − |
| 57 | A8S | 1.523 | + |
| 58 | V354C | 0.306 | − |
| 59 | V564F | 0.018 | − |
| 60 | H248S | −0.016 | − |
| 61 | I13C | 0.042 | − |
| 62 | A234Y | 1.135 | + |
| 63 | K493F | 0.566 | − |
| 64 | I10Y | 1.464 | + |
| 65 | L105V | −0.023 | − |
| 66 | K383Q | 0.112 | − |
| 67 | Y19L | −0.013 | − |
| 68 | T89L | −0.071 | − |
| 69 | K383H | 0.091 | − |
| 70 | N525S | 1.773 | + |
| 71 | L252I | 0.366 | − |
| 72 | Q491G | 1.488 | + |
| 73 | T220W | −0.096 | − |
| 74 | K254I | 0.363 | − |
| 75 | A140E | 1.255 | + |
| 76 | L291A | 0.156 | − |
| 77 | D373S | 0.335 | − |
| 78 | I195T | 0.063 | − |
| 79 | A318Y | 2.0 | ++ |
| 80 | L256I | 2.25 | ++ |
| 81 | A241C | 0.074 | − |
| 82 | A314S | 0.694 | − |
| 83 | S67A | 1.079 | + |
| 84 | T290W | 0.162 | − |
| 85 | S230M | 0.555 | − |
| 86 | Q519P | 1.699 | + |
| 87 | S288H | 3.033 | ++ |
| 88 | R376T | 1.045 | + |
| 89 | K254T | 0.578 | − |
| 90 | V354F | 0.313 | − |
| 91 | S217Q | 0.669 | − |
| 92 | A234T | 0.758 | − |
| 93 | S82T | 1.399 | + |
| 94 | I88A | 0.096 | − |
| 95 | N7T A8S Y19Q G30D S39A V40L K44H Q47R L49Y M54L | 0 | − |
| | K60R L64A Y69L R73G S82G M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D F127MRHTG (“MRHTG” disclosed as SEQ ID | | |
| | NO: 21) d128-131 A138S A140E V144T T149R Y152A A159G | | |
| | E162HFDSF (“HFDSF” disclosed as SEQ ID NO: 25) d163-166 | | |
| | A170G H189G R213M T220M S221A N227GKWSQ | | |
| | (“GKWSQ” disclosed as SEQ ID NO: 30) d228-231 Q238DVS | | |
| | d239-240 F255ILLLNV (“ILLLNV” disclosed as SEQ ID NO: 38) | | |
| | d256-260 Y268IMNR (“IMNR” disclosed as SEQ ID NO: 43) | | |
| | d269-271 I275EEMLQAIQ (“EEML QAIQ” disclosed as SEQ ID | | |
| | NO: 46) d276-282 N287TVFFA (“TVFFA” disclosed as SEQ ID | | |
| | NO: 51) d288-291 d295 L298IM T300A I304E I309L P310L | | |
| | A314S A318T I319V A328RELE (“RELE” disclosed as SEQ ID | | |
| | NO: 58) d329-331 L335R T336K T339S N341T L351V K374T | | |
| | E375P I378P Q389E N394D V398N A402L D411TENAPR | | |
| | (“TENAPR” disclosed as SEQ ID NO: 63) d412-416 L419G | | |
| | D433K H436W E443RASFA (“RASFA” disclosed as SEQ ID | | |
| | NO: 66) d444-447 D455G Y459D R461G L471K K474EDGY | | |
| | (“EDGY” disclosed as SEQ ID NO: 68) d475-477 G506RHPD | | |
| | (“RHPD” disclosed as SEQ ID NO: 69) d507-509 T513A V521P | | |
| | D540T V542E T546K Q551R d562 I565F D566VE | | |
| 96 | V481C | 2.962 | ++ |
| 97 | I426M | 0.097 | − |
| 98 | S217R | 1.605 | + |
| 99 | R477H | 0.201 | − |
| 100 | P371W | 0.284 | − |
| 101 | A229F | 0.355 | − |
| 102 | T266I | 0.07 | − |
| 103 | N12D | 1.621 | + |
| 104 | L64G | 0.053 | − |
| 105 | M479T | 0.918 | − |
| 106 | S217Y | 0.253 | − |
| 107 | G258S | 0.4 | − |
| 108 | N259C | 0.277 | − |
| 109 | F250M | 0.079 | − |
| 110 | N7T A8S D20W G30D S39A V40L K44H Q47R L49Y A52R | −0.016 | − |
| | M54L K60R L64A Y69L C72T R73G S82G M83K L87V T89V | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152I A159G E162HFDAF (“HFDAF” disclosed as SEQ ID NO: | | |
| | 26) d163-166 A170G H189G R213M S221A E224H | | |
| | N227GKWSQ (“GKWSQ” disclosed as SEQ ID NO: 30) d228- | | |
| | 231 Q238DVS d239-240 F255ILLLNVHK (“ILLLNVHK” | | |
| | disclosed as SEQ ID NO: 39) d256-262 Y268IMNR (“IMNR” | | |
| | disclosed as SEQ ID NO: 43) d269-271 L274PEEML QAIQ | | |
| | (“PEEMLQAIQ” disclosed as SEQ ID NO: 45) d275-282 | | |
| | N287TMFFA (“TMFFA” disclosed as SEQ ID NO: 54) d288-291 | | |
| | d295 L298IM T300A I304E I309L P310L A314S M316V I319V | | |
| | A328RELE (“RELE” disclosed as SEQ ID NO: 58) d329-331 | | |
| | L335R T336K T339S N341T L351V K374T E375P I378P Q389E | | |
| | R391K N394D V398N A402L D411TQNAPN (“TQNAPN” | | |
| | disclosed as SEQ ID NO: 64) d412-416 L419G D433K H436W | | |
| | E443RASFA (“RASFA” disclosed as SEQ ID NO: 66) d444-447 | | |
| | D455G Y459D R461G L471K K474EDGY (“EDGY” disclosed as | | |
| | SEQ ID NO: 68) d475-477 G506RHPD (“RHPD” disclosed as | | |
| | SEQ ID NO: 69) d507-509 T513A V521P D540T V542D T546A | | |
| | Q551R d562 I565FV | | |
| 111 | A366S | 0.305 | − |
| 112 | L320I | 0.393 | − |
| 113 | A318M | 2.432 | ++ |
| 114 | Y19Q | 0.12 | − |
| 115 | F255T | 0.076 | − |
| 116 | I225L | 0.59 | − |
| 117 | A109L | 0.022 | − |
| 118 | G258L | 3.015 | ++ |
| 119 | N7T A8S D20W G30D T36H S39A V40L K44T Q47RTYT | 0.143 | − |
| | (“RTYT” disclosed as SEQ ID NO: 17) d48-50 M54L K60R | | |
| | L64A Y69L C72T R73G S82G M83K L87VAV d88-89 | | |
| | V94CIEAA (“CIEAA” disclosed as SEQ ID NO: 18) d95-98 | | |
| | I111VTPAN (“VTPAN” disclosed as SEQ ID NO: 20) d112-115 | | |
| | S119T Q121D V129H T131G A138S A140E V144T T149R | | |
| | Y152C A159G E162HFDSF (“HFDSF” disclosed as SEQ ID NO: | | |
| | 25) d163-166 A170G H189G R213M S221A E224H | | |
| | N227GKWSQ (“GKWSQ” disclosed as SEQ ID NO: 30) d228- | | |
| | 231 Q238DVS d239-240 F255ILLHNVHK (“ILLHNVHK” | | |
| | disclosed as SEQ ID NO: 40) d256-262 Y268IMNR (“IMNR” | | |
| | disclosed as SEQ ID NO: 43) d269-271 L274PEEMLQAIQ | | |
| | (“PEEMLQAIQ” disclosed as SEQ ID NO: 45) d275-282 | | |
| | N287TVFFA (“TVFFA” disclosed as SEQ ID NO: 51) d288-291 | | |
| | d295 L298IM T300A I304E I309L P310L A314SHVRTV | | |
| | (“SHVRTV” disclosed as SEQ ID NO: 57) d315-319 A328RELE | | |
| | (“RELE” disclosed as SEQ ID NO: 58) d329-331 T336K T339S | | |
| | N341TL351V K374P E375P I378P P386A Q389E N394D | | |
| | V398N D411TENAPR (“TENAPR” disclosed as SEQ ID NO: 63) | | |
| | d412-416 L419G D433K H436W E443RASFA (“RASFA” | | |
| | disclosed as SEQ ID NO: 66) d444-447 D455G Y459D R461G | | |
| | L471R K474EDGH (“EDGH” disclosed as SEQ ID NO: 67) d475- | | |
| | 477 A508P Q509D T513A V521P D540T V542D T546A Q551R | | |
| | d562 I565F D566VE | | |
| 120 | V521P | 1.599 | + |
| 121 | A104L | 0.117 | − |
| 122 | E375P | −0.036 | − |
| 123 | S288F | 1.659 | + |
| 124 | L198S | −0.017 | − |
| 125 | T235R | 0.46 | − |
| 126 | Q509D | 0.009 | − |
| 127 | F464L | 0.617 | − |
| 128 | Y478W | 0.786 | − |
| 129 | S82E | 0.072 | − |
| 130 | L249A | 0.632 | − |
| 131 | Y197W | −0.01 | − |
| 132 | A240F | 0.273 | − |
| 133 | M503V | 0.045 | − |
| 134 | E348D | 0.461 | − |
| 135 | V354L | 0.266 | − |
| 136 | C445M | 0.224 | − |
| 137 | E224T | 0.109 | − |
| 138 | N7T A8S Y19Q S39A V40L A43G K44S Q47R L49Y A52K | −0.092 | − |
| | M54L K60R L64A S66R Y69L R73G S82N M83K L87VAV d88- | | |
| | 89 V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R F127MRITG (“MRITG” disclosed as SEQ ID NO: | | |
| | 22) d128-131 A138S A140E V144T T149R Y152A A159G | | |
| | E162RIESF (“RIESF” disclosed as SEQ ID NO: 24) d163-166 | | |
| | A170G H189G R213M T220M S221A E224N | | |
| | N227GKWSQDLK (“GKWSQDLK” disclosed as SEQ ID NO: | | |
| | 31) d228-234 Q238DISAC (“DISAC” disclosed as SEQ ID NO: | | |
| | 34) d239-242 F255ILLSNVHY (“ILLSNVHY” disclosed as SEQ | | |
| | ID NO: 41) d256-262 Y268IMPR (“IMPR” disclosed as SEQ ID | | |
| | NO: 42) d269-271 I275EEMLQAIQ (“EEML QAIQ” disclosed as | | |
| | SEQ ID NO: 46) d276-282 N287TVFHA (“TVFHA” disclosed as | | |
| | SEQ ID NO: 55) d288-291 d295 L298IM T300A I304E I309L | | |
| | P310L A314S A318S I319V A328RELE (“RELE” disclosed as | | |
| | SEQ ID NO: 58) d329-331 T336K T339S N341T E348D L351V | | |
| | K374P E375P I378P Q389E N394D V398N A402L | | |
| | D411TENAPD (“TENAPD” disclosed as SEQ ID NO: 61) d412- | | |
| | 416 L419G D433K H436W E443RASFA (“RASFA” disclosed as | | |
| | SEQ ID NO: 66) d444-447 D455G Y459D R461G L471R | | |
| | K474EDGH (“EDGH” disclosed as SEQ ID NO: 67) d475-477 | | |
| | A508P Q509D T513A V521P D540T V542D T546A Q551R | | |
| | I562V I565V D566E | | |
| 139 | S202T | 0.095 | − |
| 140 | V330L | 2.999 | ++ |
| 141 | A583Q | 1.072 | + |
| 142 | V86I | 0.346 | − |
| 143 | M200W | 0.232 | − |
| 144 | N7T A8S Y19Q D20W G30D S39A V40L K44H Q47R L49Y | −0.097 | − |
| | M54L K60R L64A Y69L C72T R73G S82A M83K L87V T89V | | |
| | V94TIEAA (“TIEAA” disclosed as SEQ ID NO: 19)d95-98 | | |
| | I111VTPAN (“VTPAN” disclosed as SEQ ID NO: 20)d112-115 | | |
| | S119T Q121D V129H T131G A138S A140E V144T T149R | | |
| | Y152C A159G E162HFDAF (“HFDAF” disclosed as SEQ ID | | |
| | NO: 26)d163-166 A170G H189G R213M T220M S221A | | |
| | N227GKWSQ (“GKWSQ” disclosed as SEQ ID NO: 30)d228- | | |
| | 231 Q238D A240S F255ILLHNVHK (“ILLHNVHK” disclosed | | |
| | as SEQ ID NO: 40)d256-262 Y268IMNR (“IMNR” disclosed as | | |
| | SEQ ID NO: 43)d269-271 I275EEML QAIQ (“EEML QAIQ” | | |
| | disclosed as SEQ ID NO: 46)d276-282 N287TFFFA (“TFFFA” | | |
| | disclosed as SEQ ID NO: 53)d288-291 d295 L298IM T300A | | |
| | I304E I309L P310L A314S M316V I319V A328RELE (“RELE” | | |
| | disclosed as SEO ID NO: 58)d329-331 T336K T339S N341T | | |
| | E348D L351V K374T E375P I378P Q389E N394D V398N | | |
| | A402L D411TENAPD (“TENAPD” disclosed as SEQ ID NO: | | |
| | 61)d412-416 L419G D433K H436W E443RASFA (“RASFA” | | |
| | disclosed as SEQ ID NO: 66)d444-447 D455G Y459D R461G | | |
| | L471R K474EDGY (“EDGY” disclosed as SEQ ID NO: 68)d475- | | |
| | 477 A508P Q509D T513A V521P D540T V542D T546A Q551R | | |
| | d562 I565F D566VE | | |
| 145 | Y268V | 0.082 | − |
| 146 | Y152A | 0.516 | − |
| 147 | S202H | −0.041 | − |
| 148 | P113L | 0.097 | − |
| 149 | N7T A8S Y19Q G30D S39A V40L K44T Q47R M54L K60R | −0.043 | − |
| | L64A Y69L R73G S82G M83K L87VAV d88-89 V94TIEAA | | |
| | (“TIEAA” disclosed as SEQ ID NO: 19)d95-98 I111VTPAN | | |
| | (“VTPAN” disclosed as SEQ ID NO: 20)d112-115 S119T Q121R | | |
| | V129H T131G A138S A140E V144T T149R Y152A A159G | | |
| | E162HFDSF (“HFDSF” disclosed as SEQ ID NO: 25)d163-166 | | |
| | A170G H189G R213M T220M S221A N227G E231QDLQ | | |
| | (“QDLQ” disclosed as SEQ ID NO: 33)d232-234 Q238DVS | | |
| | d239-240 F255I I257L P260V A262Y Y268IMPR (“IMPR” | | |
| | disclosed as SEQ ID NO: 42)d269-271 I275EEML QAIQ | | |
| | (“EEMLQAIQ” disclosed as SEQ ID NO: 46)d276-282 | | |
| | N287TSMFA (“TSMFA” disclosed as SEQ ID NO: 70) d288-291 | | |
| | d295 L298IM T300A I304E I309L P310L A314SHVRTV | | |
| | (“SHVRTV” disclosed as SEQ ID NO: 57)d315-319 N329ELE | | |
| | d330-331 L335R T336K T339S N341T L351V K374P E375P | | |
| | I378P P386A Q389E N394D V398N A402L D411TENAPR | | |
| | (“TENAPR” disclosed as SEQ ID NO: 63)d412-416 L419G | | |
| | D433K H436W E443RASFA (“RASFA” disclosed as SEQ ID | | |
| | NO: 66)d444-447 D455G Y459D R461G L471K K474EDGH | | |
| | (“EDGH” disclosed as SEQ ID NO: 67)d475-477 A508P Q509D | | |
| | T513A V521P D540T V542E T546K Q551R I562V I565V | | |
| 150 | L256V | 2.041 | ++ |
| 151 | F90C | 0.12 | − |
| 152 | E279Q | 0.172 | − |
| 153 | T290H | 0.219 | − |
| 154 | K254Y | 0.023 | − |
| 155 | I257Y | −0.037 | − |
| 156 | N7T A8S Y19Q G30D S39A V40L K44T Q47R L49Y M54L | 0.004 | − |
| | K60R L64A Y69L C72T R73G S82G M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R F127MRHTG (“MRHTG” disclosed as SEQ ID | | |
| | NO: 21) d128-131 A138S A140E V144T T149R Y152T A159G | | |
| | E162HFDSF (“HFDSF” disclosed as SEQ ID NO: 25) d163-166 | | |
| | A170G H189G R213M T220M S221A N227GKWSQQLQ | | |
| | (“GKWSQQLQ” disclosed as SEQ ID NO: 32) d228-234 Q238D | | |
| | A240S F255ILLANVHY (“ILLANVHY” disclosed as SEQ ID | | |
| | NO: 36) d256-262 Y268IMPR (“IMPR” disclosed as SEQ ID NO: | | |
| | 42) d269-271 I275EEML QAIQ (“EEML QAIQ” disclosed as SEQ | | |
| | ID NO: 46) d276-282 N287TFFFA (“TFFFA” disclosed as SEQ | | |
| | ID NO: 53) d288-291 d295 L298IM T300A I304E I309L P310L | | |
| | A314S I319V A328RELE (“RELE” disclosed as SEQ ID NO: 58) | | |
| | d329-331 T336K T339S N341T L351V K374T E375P I378P | | |
| | Q389E R391K N394D V398N A402L D411TENAPD | | |
| | (“TENAPD” disclosed as SEQ ID NO: 61) d412-416 L419G | | |
| | D433K H436W C445S V447A D455G Y459D R461G L471K | | |
| | K474EDGY (“EDGY” disclosed as SEQ ID NO: 68) d475-477 | | |
| | A508P Q509D T513A V521P D540T V542E T546K Q551R | | |
| | I562V I565V | | |
| 157 | N7T A8S Y19Q G30D S39A V40L A43G K44S Q47R L49Y | −0.037 | − |
| | M54L K60R L64A Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D F127MRHTG (“MRHTG” disclosed as SEQ ID | | |
| | NO: 21) d128-131 A138S A140E V144T T149R Y152I A159G | | |
| | E162HIDSF (“HIDSF” disclosed as SEQ ID NO: 27) d163-166 | | |
| | A170G H189G R213M T220M S221A N227GKWSQD | | |
| | (“GKWSQD” disclosed as SEQ ID NO: 28) d228-232 | | |
| | Q238DISAC (“DISAC” disclosed as SEQ ID NO: 34) d239-242 | | |
| | F2551 I257L P260V Y268IMPR (“IMPR” disclosed as SEQ ID | | |
| | NO: 42) d269-271 I275E E279QAIQ (“QAIQ” disclosed as SEQ | | |
| | ID NO: 49) d280-282 N287TVFFA (“TVFFA” disclosed as SEQ | | |
| | ID NO: 51) d288-291 d295 L298IM T300A I304E I309L P310L | | |
| | A314SHVRSV (“SHVRSV” disclosed as SEQ ID NO: 56) d315- | | |
| | 319 A328RELE (“RELE” disclosed as SEQ ID NO: 58) d329-331 | | |
| | L335Q T336K T339S N341TL351V K374T E375P I378P P386A | | |
| | Q389E R391K N394D V398N D411TENAPR (“TENAPR” | | |
| | disclosed as SEQ ID NO: 63) d412-416 L419G D433K H436W | | |
| | C445S V447A D455G Y459D R461G L471K K474EDGY | | |
| | (“EDGY” disclosed as SEQ ID NO: 68) d475-477 A508P Q509D | | |
| | T513A V521P D540T V542D T546A Q551R d562 I565F | | |
| | D566VE | | |
| 158 | A234K | 0.402 | − |
| 159 | G258P | −0.069 | − |
| 160 | A318T | 3.15 | ++ |
| 161 | R376K | 0.58 | − |
| 162 | I378S | 1.855 | + |
| 163 | D433S | 0.203 | − |
| 164 | V277M | 2.134 | ++ |
| 165 | R213M | −0.005 | − |
| 166 | S232W | 0.72 | − |
| 167 | G114L | 0.123 | − |
| 168 | V365L | 1.353 | + |
| 169 | T9K | 0.724 | − |
| 170 | D455S | 1.023 | + |
| 171 | N7K A8S Y19F G30D S39A V40L K44H Q47R L49Y M54L | −0.072 | − |
| | K60R L64A Y69L C72T R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D F127MRHTG (“MRHTG” disclosed as SEQ ID | | |
| | NO: 21) d128-131 A138S A140E V144T T149R Y152I A159G | | |
| | E162HFDSF (“HFDSF” disclosed as SEQ ID NO: 25) d163-166 | | |
| | A170G H189G R213M S221A N227GKWSQD (“GKWSQD” | | |
| | disclosed as SEQ ID NO: 28) d228-232 Q238DISAC (“DISAC” | | |
| | disclosed as SEQ ID NO: 34) d239-242 F255ILLANVHY | | |
| | (“ILLANVHY” disclosed as SEQ ID NO: 36) d256-262 | | |
| | Y268IMNR (“IMNR” disclosed as SEQ ID NO: 43) d269-271 | | |
| | L274PEEMLQAIQ (“PEEMLQAIQ” disclosed as SEQ ID NO: | | |
| | 45) d275-282 N287TVFFA (“TVFFA” disclosed as SEQ ID NO: | | |
| | 51) d288-291 d295 L298IM T300A I304E E306D I309L P310L | | |
| | A314SHVRTV (“SHVRTV” disclosed as SEQ ID NO: 57) d315- | | |
| | 319 A328RELE (“RELE” disclosed as SEQ ID NO: 58) d329-331 | | |
| | L335R T336K T339S N341T L351V K374T E375P I378P Q389E | | |
| | N394D V398N D411TENAPD (“TENAPD” disclosed as SEQ ID | | |
| | NO: 61) d412-416 L419G D433K H436W E443RASFA | | |
| | (“RASFA” disclosed as SEQ ID NO: 66) d444-447 D455G | | |
| | Y459D R461G L471K K474EDGY (“EDGY” disclosed as SEQ | | |
| | ID NO: 68) d475-477 G506RHPD (“RHPD” disclosed as SEQ ID | | |
| | NO: 69) d507-509 T513A V521P D540T V542D T546A Q551R | | |
| | I562V I565V | | |
| 172 | F90M | 1.5 | + |
| 173 | I244M | 0.118 | − |
| 174 | P260F | 0.54 | − |
| 175 | L87V | 0.861 | − |
| 176 | T331M | 0.053 | − |
| 177 | A318S | 1.326 | + |
| 178 | L351V | −0.015 | − |
| 179 | V431I | 0.941 | − |
| 180 | S201F | −0.091 | − |
| 181 | A103G | 0.131 | − |
| 182 | E35P | −0.054 | − |
| 183 | G258M | 2.822 | ++ |
| 184 | L256H | −0.018 | − |
| 185 | A262M | 0.3 | − |
| 186 | A196T | −0.057 | − |
| 187 | T149R | 0.661 | − |
| 188 | S223N | 1.263 | + |
| 189 | I4M N7T A8S Y19Q G30D S39A V40L A43G K44T Q47R L49Y | 0.053 | − |
| | A52K M54L K60R L64A Y69L C72T R73G E78V S82N M83K | | |
| | L87VA d89 V94TIEAA (“TIEAA” disclosed as SEQ ID NO: 19) | | |
| | d95-98 I111VTPAN (“VTPAN” disclosed as SEQ ID NO: 20) | | |
| | d112-115 S119T Q121D F127MRHTG (“MRHTG” disclosed as | | |
| | SEQ ID NO: 21) d128-131 A138S A140E V144T T149R Y152T | | |
| | A159G E162RIESF (“RIESF” disclosed as SEQ ID NO: 24) d163- | | |
| | 166 A170G H189G R213M T220M S221A N227GKWSQDLK | | |
| | (“GKWSQDLK” disclosed as SEQ ID NO: 31) d228-234 | | |
| | Q238DISAC (“DISAC” disclosed as SEQ ID NO: 34) d239-242 | | |
| | F255I I257L P260V Y268IMNR (“IMNR” disclosed as SEQ ID | | |
| | NO: 43) d269-271 I275EEMLQAIQ (“EEML QAIQ” disclosed as | | |
| | SEQ ID NO: 46) d276-282 N287TMFHA (“TMFHA” disclosed as | | |
| | SEQ ID NO: 71) d288-291 d295 L298IM T300A I304E I309L | | |
| | P310L A314SHVRSV (“SHVRSV” disclosed as SEQ ID NO: 56) | | |
| | d315-319 A328RELE (“RELE” disclosed as SEQ ID NO: 58) | | |
| | d329-331 T336K T339S N341T L351V K374T E375P I378P | | |
| | P386A Q389E N394D V398N A402L L410NTENNPD | | |
| | (“NTENNPD” disclosed as SEQ ID NO: 72) d411-416 L419G | | |
| | D433K H436W E443RASFA (“RASFA” disclosed as SEQ ID | | |
| | NO: 66) d444-447 D455G Y459D R461G L471K K474EDGY | | |
| | (“EDGY” disclosed as SEQ ID NO: 68) d475-477 A508P Q509D | | |
| | T513A V521P D540T V542D T546A Q551R I562V I565V | | |
| | D566E | | |
| 190 | G117S | 1.546 | + |
| 191 | A138D | 1.137 | + |
| 192 | K254H | 0.546 | − |
| 193 | R213V | 0.014 | − |
| 194 | L105W | 0.039 | − |
| 195 | K44S | 0.725 | − |
| 196 | I225A | 0.128 | − |
| 197 | I480V | 2.885 | ++ |
| 198 | I10W | 1.288 | + |
| 199 | S288M | 0.607 | − |
| 200 | D370Y | 0.068 | − |
| 201 | N341H | −0.048 | − |
| 202 | I296M | 1.301 | + |
| 203 | R317N | −0.096 | − |
| 204 | F250A | 0.091 | − |
| 205 | C445T | 0.283 | − |
| 206 | V472I | 0.216 | − |
| 207 | P310L | 0.06 | − |
| 208 | I275D | 0.191 | − |
| 209 | L107A | 0.029 | − |
| 210 | Q416D | 0.887 | − |
| 211 | S253T | 0.202 | − |
| 212 | Y357W | 0.038 | − |
| 213 | I378Y | 0.987 | − |
| 214 | K383T | 0.047 | − |
| 215 | V297M | 0.085 | − |
| 216 | R73G | 0.649 | − |
| 217 | N7T A8S Y19Q D20W G30D S39A V40L K44H | −0.181 | − |
| | Q47RTYTQKEL (“RTYTQKEL” disclosed as SEQ ID NO: 15) | | |
| | d48-54 K60R L64A Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152A A159G E162HFDAF (“HFDAF” disclosed as SEQ ID | | |
| | NO: 26) d163-166 A170G H189G R213M T220M S221A I225A | | |
| | N227G E231Q S232D Q238DISAC (“DISAC” disclosed as SEQ | | |
| | ID NO: 34) d239-242 F255I I257L P260V Y268IMNR (“IMNR” | | |
| | disclosed as SEQ ID NO: 43) d269-271 I275KEML QAIQ | | |
| | (“KEML QAIQ” disclosed as SEQ ID NO: 48) d276-282 | | |
| | N287TVFFA (“TVFFA” disclosed as SEQ ID NO: 51) d288-291 | | |
| | d295 L298IM T300A I304E I309L P310L A314SHVRTV | | |
| | (“SHVRTV” disclosed as SEQ ID NO: 57) d315-319 A328RELE | | |
| | (“RELE” disclosed as SEQ ID NO: 58) d329-331 L335Q T336K | | |
| | T339S N341T L351V K374P E375P I378P Q389E N394D V398N | | |
| | A402L D411TENAPE (“TENAPE” disclosed as SEQ ID NO: 73) | | |
| | d412-416 L419G D433K H436W E443RASFA (“RASFA” | | |
| | disclosed as SEQ ID NO: 66) d444-447 D455G Y459D R461G | | |
| | L471K K474EDGF (“EDGF” disclosed as SEQ ID NO: 74) d475- | | |
| | 477 A508P Q509D T513A V521P D540T V542E T546K Q551R | | |
| | I562V I565V D566E | | |
| 218 | N7Q | 0.425 | − |
| 219 | T265C | 0.881 | − |
| 220 | P528A | 0.959 | − |
| 221 | R194D | −0.065 | − |
| 222 | R477F | 0.048 | − |
| 223 | V481I | 1.147 | + |
| 224 | N7T A8S Y19Q G30D S39A V40L K44S Q47R L49Y M54L | −0.06 | − |
| | K60R L64A Y69L R73G S82A M83K L87VAV d88-89 | | |
| | V94TIEAA (“TIEAA” disclosed as SEQ ID NO: 19) d95-98 | | |
| | I111V G114A S115N S119T Q121D F127MRHTG (“MRHTG” | | |
| | disclosed as SEQ ID NO: 21) d128-131 A138S A140E V144T | | |
| | T149R Y152T A159G E162HFDAF (“HFDAF” disclosed as SEQ | | |
| | ID NO: 26) d163-166 A170G H189G R213M T220M S221A | | |
| | N227GKWSQDLQ (“GKWSQDLQ” disclosed as SEQ ID NO: | | |
| | 75) d228-234 Q238DISAC (“DISAC” disclosed as SEQ ID NO: | | |
| | 34) d239-242 F255ILLANV (“ILLANV” disclosed as SEQ ID | | |
| | NO: 35) d256-260 Y268IMPR (“IMPR” disclosed as SEQ ID NO: | | |
| | 42) d269-271 I275EEML QAIQ (“EEML QAIQ” disclosed as SEQ | | |
| | ID NO: 46) d276-282 N287TSFFA (“TSFFA” disclosed as SEQ | | |
| | ID NO: 52) d288-291 d295 L298IM T300A I304E I309L P310L | | |
| | A314SHVRTV (“SHVRTV” disclosed as SEQ ID NO: 57) d315- | | |
| | 319 N329ELE d330-331 L335R T336K T339S N341H E348D | | |
| | L351V K374T E375P I378P P386A Q389E N394D V398N | | |
| | A402L D411TQNAPR (“TQNAPR” disclosed as SEQ ID NO: 62) | | |
| | d412-416 L419G D433K H436W E443RASFA (“RASFA” | | |
| | disclosed as SEQ ID NO: 66) d444-447 D455G Y459D R461G | | |
| | L471R K474EDGY (“EDGY” disclosed as SEQ ID NO: 68) d475- | | |
| | 477 A508P Q509D T513A V521P D540T V542E T546K Q551R | | |
| | I562V I565V D566E | | |
| 225 | S253Q | 1.934 | + |
| 226 | Q238P | 0.092 | − |
| 227 | I195P | 0.066 | − |
| 228 | N7K A8S Y19Q D20W G30D S39A V40L K44H Q47R L49Y | −0.07 | − |
| | M54L K60R L64A Y69L C72T R73G E78V S82N M83K | | |
| | L87VAV d88-89 V94TIEAA (“TIEAA” disclosed as SEQ ID NO: | | |
| | 19) d95-98 I111VTPAN (“VTPAN” disclosed as SEQ ID NO: 20) | | |
| | d112-115 S119T Q121R V129H T131G A138S A140E V144T | | |
| | T149R Y1521 A159G E162HFDAF (“HFDAF” disclosed as SEQ | | |
| | ID NO: 26) d163-166 A170G H189G R213M T220M S221A | | |
| | N227GKWSQDLQ (“GKWSQDLQ” disclosed as SEQ ID NO: | | |
| | 75) d228-234 Q238DISAC (“DISAC” disclosed as SEQ ID NO: | | |
| | 34) d239-242 F255ILLANV (“ILLANV” disclosed as SEQ ID | | |
| | NO: 35) d256-260 Y268IMNR (“IMNR” disclosed as SEQ ID | | |
| | NO: 43) d269-271 I275E E279RAIQ (“RAIQ” disclosed as SEQ | | |
| | ID NO: 50) d280-282 N287TSFFA (“TSFFA” disclosed as SEQ | | |
| | ID NO: 52) d288-291 d295 L298IM T300A I304M E306D I309L | | |
| | P310L A314SHVRTV (“SHVRTV” disclosed as SEQ ID NO: 57) | | |
| | d315-319 A328RELE (“RELE” disclosed as SEQ ID NO: 58) | | |
| | d329-331 L335R T336K T339S N341T L351V K374T E375P | | |
| | I378P Q389E R391K N394D V398N A402L D411TENAPR | | |
| | (“TENAPR” disclosed as SEQ ID NO: 63) d412-416 L419G | | |
| | D433K H436W C445S V447A D455G Y459D R461G L471R | | |
| | K474EDGH (“EDGH” disclosed as SEQ ID NO: 67) d475-477 | | |
| | G506RHPD (“RHPD” disclosed as SEQ ID NO: 69) d507-509 | | |
| | T513A V521P D540T V542D T546A Q551R d562 I565F | | |
| | D566VE | | |
| 229 | T290C | 0.618 | − |
| 230 | N7T A8S D20W G30D S39A V40L A43G K44S Q47R L49Y | −0.034 | − |
| | M54L K60R L64A Y69L R73G E78V S82N M83K L87VAV | | |
| | d88-89 V94TIEAA (“TIEAA” disclosed as SEQ ID NO: 19) d95- | | |
| | 98 I111VTPAN (“VTPAN” disclosed as SEQ ID NO: 20) d112- | | |
| | 115 S119T Q121D V1291 T131G A138S A140E V144T T149R | | |
| | Y152A A159G E162RIESF (“RIESF” disclosed as SEQ ID NO: | | |
| | 24) d163-166 A170G H189G R213M S221A E224H N227G | | |
| | E231Q S232D Q238DISAC (“DISAC” disclosed as SEQ ID NO: | | |
| | 34) d239-242 F255ILLANVHY (“ILLANVHY” disclosed as SEQ | | |
| | ID NO: 36) d256-262 Y268IMPR (“IMPR” disclosed as SEQ ID | | |
| | NO: 42) d269-271 I275KEMLQAIQ (“KEMLQAIQ” disclosed as | | |
| | SEQ ID NO: 48) d276-282 N287TVFFA (“TVFFA” disclosed as | | |
| | SEQ ID NO: 51) d288-291 d295 L298IM T300A I304E I309L | | |
| | P310L A314S A318T I319V A328RELE (“RELE” disclosed as | | |
| | SEQ ID NO: 58) d329-331 L335R T336K T339S N341T L351V | | |
| | K374T E375P I378P P386A Q389E R391K N394D V398N | | |
| | A402L D411TENAPD (“TENAPD” disclosed as SEQ ID NO: 61) | | |
| | d412-416 L419G D433K H436W E443RASFA (“RASFA” | | |
| | disclosed as SEQ ID NO: 66) d444-447 D455G Y459G R461G | | |
| | L471K K474EDGY (“EDGY” disclosed as SEQ ID NO: 68) | | |
| | d475-477 A508P Q509D T513A V521P D540T V542E T546K | | |
| | Q551R I562V I565V D566E | | |
| 231 | K493H | 1.464 | + |
| 232 | V365I | 2.57 | ++ |
| 233 | R73F | 0.962 | − |
| 234 | L107C | −0.05 | − |
| 235 | Q238N | 0.326 | − |
| 236 | T116A | 0.485 | − |
| 237 | I4M | 0.089 | − |
| 238 | A527P | −0.011 | − |
| 239 | Q389R | −0.051 | − |
| 240 | A229S | 0.403 | − |
| 241 | A366V | 1.675 | + |
| 242 | R477N | 0.711 | − |
| 243 | S363A | 0.91 | − |
| 244 | I111V | 1.502 | + |
| 245 | L334A | 0.073 | − |
| 246 | S232A | 0.985 | − |
| 247 | F90W | −0.119 | − |
| 248 | L214V | −0.015 | − |
| 249 | H74K | 1.244 | + |
| 250 | A241M | 0.21 | − |
| 251 | L359M | 0.499 | − |
| 252 | N387G | 0.011 | − |
| 253 | S50T | 0.111 | − |
| 254 | T418P | 0.205 | − |
| 255 | T9A | 0.171 | − |
| 256 | N7T A8S D20W G30D S39A V40L K44H Q47RTYTQKEL | −0.051 | − |
| | (“RTYTQKEL” disclosed as SEQ ID NO: 15)d48-54 K60R L64A | | |
| | Y69L C72T R73G S82N M83K L87V T89V V94TIEAA | | |
| | (“TIEAA” disclosed as SEQ ID NO: 19)d95-98 I111VTPAN | | |
| | (“VTPAN” disclosed as SEQ ID NO: 20)d112-115 S119T Q121R | | |
| | F127MRHTG (“MRHTG” disclosed as SEQ ID NO: 21)d128-131 | | |
| | A138S A140E V144T T149R Y152T A159G E162HFDSF | | |
| | (“HFDSF” disclosed as SEQ ID NO: 25)d163-166 A170G H189G | | |
| | R213M S221A E224H N227GKWSQ (“GKWSQ” disclosed as | | |
| | SEQ ID NO: 30)d228-231 A234K Q238D A240S | | |
| | F255ILLSNVHY (“ILLSNVHY” disclosed as SEQ ID NO: | | |
| | 41)d256-262 Y268IMNR (“IMNR” disclosed as SEQ ID NO: | | |
| | 43)d269-271 I275EEMLQAIQ (“EEMLQAIQ” disclosed as SEQ | | |
| | ID NO: 46)d276-282 N287TMFHA (“TMFHA” disclosed as SEQ | | |
| | ID NO: 71) d288-291 d295 L298IM T300A I304E I309L P310L | | |
| | A314S A318S I319V A328RELE (“RELE” disclosed as SEQ ID | | |
| | NO: 58)d329-331 L335R T336K T339S N341T L351V K374T | | |
| | E375P I378P Q389E R391K N394D V398N D411TENAPR | | |
| | (“TENAPR” disclosed as SEQ ID NO: 63)d412-416 L419G | | |
| | D433K H436W E443RASFA (“RASFA” disclosed as SEQ ID | | |
| | NO: 66)d444-447 D455G Y459D R461G L471R K474EDGH | | |
| | (“EDGH” disclosed as SEQ ID NO: 67)d475-477 G506RHPD | | |
| | (“RHPD” disclosed as SEQ ID NO: 69)d507-509 T513A V521P | | |
| | D540T V542E T546K Q551R I562V I565V D566E | | |
| 257 | N7T A8S D20W G30D S39A V40L K44H Q47R L49Y M54L | 0.032 | − |
| | K60R L64A Y69L C72T R73G S82N M83K L87VAV d88-89 | | |
| | V94TIEAA (“TIEAA” disclosed as SEQ ID NO: 19) d95-98 | | |
| | I111V G114A S115N S119T Q121D F127MRHTG (“MRHTG” | | |
| | disclosed as SEQ ID NO: 21) d128-131 A138S A140E V144T | | |
| | T149R Y152C A159G E162HFDAF (“HFDAF” disclosed as SEQ | | |
| | ID NO: 26) d163-166 A170G H189G R213M S221A E224H | | |
| | N227G E231Q S232Q Q238D A240S F255ILLANLHY | | |
| | (“ILLANLHY” disclosed as SEQ ID NO: 76) d256-262 | | |
| | Y268IMNR (“IMNR” disclosed as SEQ ID NO: 43) d269-271 | | |
| | I275EEML QAIQ (“EEML QAIQ” disclosed as SEQ ID NO: 46) | | |
| | d276-282 N287TFFFA (“TFFFA” disclosed as SEQ ID NO: 53) | | |
| | d288-291 d295 L298IM T300A I304E I309L P310L A314S I319V | | |
| | N329ELE d330-331 L335R T336K T339S N341T L351V K374T | | |
| | E375P I378P P386A Q389E R391K N394D V398N A402L | | |
| | D411TENAPD (“TENAPD” disclosed as SEQ ID NO: 61) d412- | | |
| | 416 L419G D433K H436W E443RASFA (“RASFA” disclosed as | | |
| | SEQ ID NO: 66) d444-447 D455G Y459D R461G L471K | | |
| | K474EDGY (“EDGY” disclosed as SEQ ID NO: 68) d475-477 | | |
| | A508P Q509D T513A V521P D540T V542E T546K Q551R d562 | | |
| | I565F D566VE | | |
| 258 | D20W | 0.017 | − |
| 259 | A318C | 3.094 | ++ |
| 260 | V398D | 0.903 | − |
| 261 | N7K A8S Y19Q G30D S39A V40L A43G K44S Q47R L49Y | 0.047 | − |
| | A52K M54L K60R L64A Y69L C72T R73G E78V S82N M83K | | |
| | L87VAV d88-89 V94TIEAA (“TIEAA” disclosed as SEQ ID NO: | | |
| | 19) d95-98 I111VTPAN (“VTPAN” disclosed as SEQ ID NO: 20) | | |
| | d112-115 S119T Q121D V129H T131G A138S A140E V144T | | |
| | T149R Y152T A159G E162RIESF (“RIESF” disclosed as SEQ ID | | |
| | NO: 24) d163-166 A170G H189G R213M T220M S221A | | |
| | N227GKWSQSY (“GKWSQSY” disclosed as SEQ ID NO: 29) | | |
| | d228-233 Q238DISAC (“DISAC” disclosed as SEQ ID NO: 34) | | |
| | d239-242 F255ILLLNVHY (“ILLLNVHY” disclosed as SEQ ID | | |
| | NO: 37) d256-262 Y268IMPR (“IMPR” disclosed as SEQ ID NO: | | |
| | 42) d269-271 I275EEML QAIQ (“EEML QAIQ” disclosed as SEQ | | |
| | ID NO: 46) d276-282 N287TMFFA (“TMFFA” disclosed as SEQ | | |
| | ID NO: 54) d288-291 d295 L298IM T300A I304E I309L P310L | | |
| | A314SHVRSV (“SHVRSV” disclosed as SEQ ID NO: 56) d315- | | |
| | 319 N329ELE d330-331 L335RKRMSGT (“RKRMSGT” | | |
| | disclosed as SEQ ID NO: 77) d336-341 L351V K374T E375P | | |
| | I378P Q389E N394D V398N A402L D411TQNAPR (“TQNAPR” | | |
| | disclosed as SEQ ID NO: 62) d412-416 L419G D433K H436W | | |
| | E443RASFA (“RASFA” disclosed as SEQ ID NO: 66) d444-447 | | |
| | D455G Y459D R461G L471R K474EDGY (“EDGY” disclosed as | | |
| | SEQ ID NO: 68) d475-477 A508P Q509D T513A V521P D540T | | |
| | V542E T546K Q551R I562V I565V | | |
| 262 | I319C | 0.018 | − |
| 263 | K415P | 0.262 | − |
| 264 | V472F | −0.045 | − |
| 265 | D373H | 0.023 | − |
| 266 | H101F | −0.035 | − |
| 267 | S226C | 0.271 | − |
| 268 | Q475D | 0.475 | − |
| 269 | F369H | 1.012 | + |
| 270 | A402L | −0.052 | − |
| 271 | D167L | 0.001 | − |
| 272 | T513A | 3.036 | ++ |
| 273 | A262K | 1.205 | + |
| 274 | H462R | 2.264 | ++ |
| 275 | V277Y | 0.025 | − |
| 276 | Q491R | 1.204 | + |
| 277 | A324S | 0.035 | − |
| 278 | S311L | −0.028 | − |
| 279 | S202A | −0.074 | − |
| 280 | D370R | −0.02 | − |
| 281 | Y268M | 0.272 | − |
| 282 | T89I | −0.001 | − |
| 283 | Y268T | −0.103 | − |
| 284 | A527T | 0.329 | − |
| 285 | K523Q | 1.181 | + |
| 286 | N7K A8S D20W G30D S39A V40L K44H Q47R L49Y M54L | 0.106 | − |
| | K60R L64A Y69L C72T R73G S82A M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D F127MRHTG (“MRHTG” disclosed as SEQ ID | | |
| | NO: 21) d128-131 A138S A140E V144T T149R Y1521 A159G | | |
| | E162HFDSF (“HFDSF” disclosed as SEQ ID NO: 25) d163-166 | | |
| | A170G H189G R213M S221A E224H N227G E231Q S232A | | |
| | Q238DISAC (“DISAC” disclosed as SEQ ID NO: 34) d239-242 | | |
| | F255ILLLNVHM (“ILLLNVHM” disclosed as SEQ ID NO: 78) | | |
| | d256-262 Y268IMNR (“IMNR” disclosed as SEQ ID NO: 43) | | |
| | d269-271 L274PEEMLRAIQ (“PEEMLRAIQ” disclosed as SEQ | | |
| | ID NO: 44) d275-282 N287TVFFA (“TVFFA” disclosed as SEQ | | |
| | ID NO: 51) d288-291 d295 L298IM T300A I304E I309L P310L | | |
| | A314SHVRSV (“SHVRSV” disclosed as SEQ ID NO: 56) d315- | | |
| | 319 A328RELE (“RELE” disclosed as SEQ ID NO: 58) d329-331 | | |
| | L335RKRMSGT (“RKRMSGT” disclosed as SEQ ID NO: 77) | | |
| | d336-341 L351V K374T E375P I378P P386A Q389E N394D | | |
| | V398N A402L D411TENAPR (“TENAPR” disclosed as SEQ ID | | |
| | NO: 63) d412-416 L419G D433K H436W E443RASFA | | |
| | (“RASFA” disclosed as SEQ ID NO: 66) d444-447 D455G | | |
| | Y459D R461G L471K K474EDGY (“EDGY” disclosed as SEQ | | |
| | ID NO: 68) d475-477 A508P Q509D T513A V521P D540T | | |
| | V542E T546K Q551K d562 I565F D566VE | | |
| 287 | V542E | 0.527 | − |
| 288 | H261M | 0.579 | − |
| 289 | T235K | 0.695 | I |
| 290 | L299M | 0.228 | − |
| 291 | E372K | 1.262 | + |
| 292 | I275A | 0.574 | − |
| 293 | N7K A8S Y19F D20W G30D S39A V40L K44S Q47R L49Y | 0.005 | − |
| | M54L K60R L64A Y69L C72T R73G S82G M83K L87VAV | | |
| | d88-89 V94TIEAA (“TIEAA” disclosed as SEQ ID NO: 19) d95- | | |
| | 98 I111VTPAN (“VTPAN” disclosed as SEQ ID NO: 20) d112- | | |
| | 115 S119T Q121D F127MRHTG (“MRHTG” disclosed as SEQ | | |
| | ID NO: 21) d128-131 A138S A140E V144T T149R Y152A | | |
| | A159G E162HFDSF (“HFDSF” disclosed as SEQ ID NO: 25) | | |
| | d163-166 A170G H189G R213M S221A E224H N227GKWSQD | | |
| | (“GKWSQD” disclosed as SEQ ID NO: 28) d228-232 Q238DVS | | |
| | d239-240 F255I I257L P260V Y268IMPR (“IMPR” disclosed as | | |
| | SEQ ID NO: 42) d269-271 I275EEMLQAIQ (“EEML QAIQ” | | |
| | disclosed as SEQ ID NO: 46) d276-282 N287TVFFA (“TVFFA” | | |
| | disclosed as SEQ ID NO: 51) d288-291 d295 L298IM T300A | | |
| | I304E I309L P310L A314SHVRSV (“SHVRSV” disclosed as | | |
| | SEQ ID NO: 56) d315-319 A328RELE (“RELE” disclosed as | | |
| | SEQ ID NO: 58) d329-331 L335R T336K T339S N341T L351V | | |
| | K374P E375P I378P P386A Q389E N394D V398N A402L | | |
| | E412ENAPR (“ENAPR” disclosed as SEQ ID NO: 79) d413-416 | | |
| | L419G D433K H436W E443RASFA (“RASFA” disclosed as SEQ | | |
| | ID NO: 66) d444-447 D455G Y459D R461G L471R K474EDGH | | |
| | (“EDGH” disclosed as SEQ ID NO: 67) d475-477 A508P Q509D | | |
| | T513A V521P D540T V542D T546A Q551R d562 I565F | | |
| | D566VE | | |
| 294 | H101M | −0.019 | − |
| 295 | V297Q | 0.082 | − |
| 296 | H189E | 0.661 | − |
| 297 | Q430T | −0.043 | − |
| 298 | S253N | 0.112 | − |
| 299 | I353V | 1.033 | − |
| 300 | H261W | 0.593 | − |
| 301 | N7K A8S Y19Q G30D S39A V40L K44S Q47R L49Y A52K | 0.05 | − |
| | M54L K60R L64A Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152C A159G E162HFDAF (“HFDAF” disclosed as SEQ ID | | |
| | NO: 26) d163-166 A170G H189G R213M T220M S221A N227G | | |
| | E231Q S232D Q238D A240S F255ILLANV (“ILLANV” | | |
| | disclosed as SEQ ID NO: 35) d256-260 Y268IMPR (“IMPR” | | |
| | disclosed as SEQ ID NO: 42) d269-271 I275KEML QAIQ | | |
| | (“KEMLQAIQ” disclosed as SEQ ID NO: 48) d276-282 | | |
| | N287THFFA (“THFFA” disclosed as SEQ ID NO: 80) d288-291 | | |
| | d295 L298IM T300A I304E I309L P310L A314SHVRSV | | |
| | (“SHVRSV” disclosed as SEQ ID NO: 56) d315-319 A328RELE | | |
| | (“RELE” disclosed as SEQ ID NO: 58) d329-331 L335R T336K | | |
| | T339S N341H E348Y L351V K374T E375P I378P P386A Q389E | | |
| | R391K N394D V398N A402L E412ENAPR (“ENAPR” disclosed | | |
| | as SEQ ID NO: 79) d413-416 L419G D433K H436W | | |
| | E443RASFA (“RASFA” disclosed as SEQ ID NO: 66) d444-447 | | |
| | D455G Y459D R461G L471R K474EDGH (“EDGH” disclosed as | | |
| | SEQ ID NO: 67) d475-477 G506RHPD (“RHPD” disclosed as | | |
| | SEQ ID NO: 69) d507-509 T513A V521P D540T V542D T546A | | |
| | Q551R I562V I565V D566E | | |
| 302 | T290F | 0.101 | − |
| 303 | S288Y | 3.109 | ++ |
| 304 | Q389K | 0.046 | − |
| 305 | G114V | −0.055 | − |
| 306 | S217H | 0.941 | − |
| 307 | G524A | 0.307 | − |
| 308 | D370T | 0.108 | − |
| 309 | A242G | −0.075 | − |
| 310 | A527V | 0.8 | − |
| 311 | N7K A8S Y19Q G30D S39A V40L K44S Q47R L49Y M54L | −0.07 | − |
| | K60R L64A Y69L C72T R73G S82A M83K L87VAV d88-89 | | |
| | V94TIEAA (“TIEAA” disclosed as SEQ ID NO: 19)d95-98 | | |
| | I111VTPAN (“VTPAN” disclosed as SEQ ID NO: 20)d112-115 | | |
| | S119T Q121D V129H T131G A138S A140E V144T T149R | | |
| | Y152C A159G E162HFDSF (“HFDSF” disclosed as SEQ ID NO: | | |
| | 25)d163-166 A170G H189G R213M T220M S221A N227G | | |
| | E231Q S232D Q238DVSAC (“DVSAC” disclosed as SEQ ID | | |
| | NO: 81) d239-242 F255ILLANVHY (“ILLANVHY” disclosed as | | |
| | SEQ ID NO: 36)d256-262 Y268IMPR (“IMPR” disclosed as SEQ | | |
| | ID NO: 42)d269-271 I275EEMLRAIQ (“EEMLRAIQ” disclosed | | |
| | as SEQ ID NO: 47)d276-282 N287TSFFA (“TSFFA” disclosed as | | |
| | SEQ ID NO: 52)d288-291 d295 L298IM T300A I304E I309L | | |
| | P310L A314SHVRTV (“SHVRTV” disclosed as SEQ ID NO: | | |
| | 57)d315-319 A328RELE (“RELE” disclosed as SEQ ID NO: | | |
| | 58)d329-331 L335Q T336K L351V K374T I378P P386A Q389E | | |
| | R391K N394D V398N A402L D411TENAPR (“TENAPR” | | |
| | disclosed as SEQ ID NO: 63)d412-416 L419G D433K H436W | | |
| | E443KASFA (“KASFA” disclosed as SEQ ID NO: 82) d444-447 | | |
| | D455G Y459D R461G L471K K474EDGY (“EDGY” disclosed | | |
| | as SEQ ID NO: 68)d475-477 G506RHPD (“RHPD” disclosed as | | |
| | SEQ ID NO: 69)d507-509 T513A V521P D540T V542E T546K | | |
| | Q551R I562V I565V | | |
| 312 | L424M | 0.745 | − |
| 313 | I225F | −0.024 | − |
| 314 | L351C | 0.16 | − |
| 315 | S82A | 0.401 | − |
| 316 | V297I | 1.642 | + |
| 317 | L249V | 1.379 | + |
| 318 | N394D | 1.393 | + |
| 319 | E224Q | 1.414 | + |
| 320 | S82R | 0.52 | − |
| 321 | L419G | 0.185 | − |
| 322 | P364C | 0.423 | − |
| 323 | A240H | 1.085 | + |
| 324 | A318Q | 2.558 | ++ |
| 325 | K150I | 1.158 | + |
| 326 | N7T A8S Y19Q G30D S39A V40L A43G K44T Q47R L49Y | −0.074 | − |
| | A52K M54L K60R L64A Y69L C72T R73G S82N M83K | | |
| | L87VAV d88-89 V94TIEAA (“TIEAA” disclosed as SEQ ID NO: | | |
| | 19) d95-98 I111VTPAN (“VTPAN” disclosed as SEQ ID NO: 20) | | |
| | d112-115 S119T Q121D F127MRHTG (“MRHTG” disclosed as | | |
| | SEQ ID NO: 21) d128-131 A138S A140E V144T T149R Y152I | | |
| | A159G E162HFDSF (“HFDSF” disclosed as SEQ ID NO: 25) | | |
| | d163-166 A170G H189G R213M T220M S221A N227G E231H | | |
| | Q238D A240S F255ILLHNVHM (“ILLHNVHM” disclosed as | | |
| | SEQ ID NO: 83) d256-262 Y268IMNR (“IMNR” disclosed as | | |
| | SEQ ID NO: 43) d269-271 L274PEEMLRAIQ (“PEEMLRAIQ” | | |
| | disclosed as SEQ ID NO: 44) d275-282 N287TFFFA (“TFFFA” | | |
| | disclosed as SEQ ID NO: 53) d288-291 d295 L298LM T300A | | |
| | I304E I309L P310L A314S M316V I319V A328RELE (“RELE” | | |
| | disclosed as SEQ ID NO: 58) d329-331 L335R T336K T339S | | |
| | N341H L351V K374P E375P I378P P386A Q389E N394D | | |
| | V398N A402L L410NTENAPR (“NTENAPR” disclosed as SEQ | | |
| | ID NO: 59) d411-416 L419G D433K H436W E443RASFA | | |
| | (“RASFA” disclosed as SEQ ID NO: 66) d444-447 D455G | | |
| | Y459D R461G L471R K474EDGH (“EDGH” disclosed as SEQ | | |
| | ID NO: 67) d475-477 G506RHPD (“RHPD” disclosed as SEQ ID | | |
| | NO: 69) d507-509 T513A V521P D540T V542E T546K Q551R | | |
| | d562 I565FV | | |
| 327 | I111A | 0.069 | − |
| 328 | A264G | 0.335 | − |
| 329 | K44T | 0.86 | − |
| 330 | N7T A8S D20W G30D T36H S39A V40L K44H Q47RTYT | 0.06 | − |
| | (“RTYT” disclosed as SEQ ID NO: 17) d48-50 M54L K60R | | |
| | L64A Y69L C72T R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D F127MRHTG (“MRHTG” disclosed as SEQ ID | | |
| | NO: 21) d128-131 A138S A140E V144T T149R Y152C A159G | | |
| | E162HFDSF (“HFDSF” disclosed as SEQ ID NO: 25) d163-166 | | |
| | A170G H189G R213M S221A N227GKWSQDLK | | |
| | (“GKWSQDLK” disclosed as SEQ ID NO: 31) d228-234 Q238D | | |
| | A240S F255ILLSNVHY (“ILLSNVHY” disclosed as SEQ ID | | |
| | NO: 41) d256-262 Y268IMNR (“IMNR” disclosed as SEQ ID | | |
| | NO: 43) d269-271 L274PEEMLQAIQ (“PEEML QAIQ” disclosed | | |
| | as SEQ ID NO: 45) d275-282 N287TMFHA (“TMFHA” disclosed | | |
| | as SEQ ID NO: 71) d288-291 d295 L298IM T300A I304E I309L | | |
| | P310L A314S A318S I319V A328RELE (“RELE” disclosed as | | |
| | SEQ ID NO: 58) d329-331 T336K L351V K374P E375P I378P | | |
| | Q389E N394D V398N A402L D411TENAPR (“TENAPR” | | |
| | disclosed as SEQ ID NO: 63) d412-416 L419G D433K H436W | | |
| | E443RASFA (“RASFA” disclosed as SEQ ID NO: 66) d444-447 | | |
| | D455G Y459D R461G L471R K474EDGH (“EDGH” disclosed as | | |
| | SEQ ID NO: 67) d475-477 A508P Q509D T513A V521P D540T | | |
| | V542D T546A Q551R d562 I565F D566VE | | |
| 331 | A328H | 1.619 | + |
| 332 | I378P | 1.529 | + |
| 333 | T9S | 0.709 | − |
| 334 | V330T | 0.262 | − |
| 335 | S388M | −0.01 | − |
| 336 | L233W | 4.745 | ++ |
| 337 | T339S | 0.148 | − |
| 338 | L471K | 0.263 | − |
| 339 | F464Y | 0.415 | − |
| 340 | L105H | −0.108 | − |
| 341 | S119T | 1.165 | + |
| 342 | S202Q | 0.052 | − |
| 343 | N7T A8S Y19Q D20W G30D S39A V40L K44T Q47R L49Y | −0.053 | − |
| | M54L K60R L64G Y69L R73G S82A M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R F127MRHTG (“MRHTG” disclosed as SEQ ID | | |
| | NO: 21) d128-131 A138S A140E V144T T149R Y152A A159G | | |
| | E162R L163I L166F A170G H189G R213M T220M S221A | | |
| | N227GKWSQ (“GKWSQ” disclosed as SEQ ID NO: 30) d228- | | |
| | 231 Q238D A240S F255ILLLNV (“ILLLNV” disclosed as SEQ | | |
| | ID NO: 38) d256-260 Y268IMPR (“IMPR” disclosed as SEQ ID | | |
| | NO: 42) d269-271 L274PEEMLQAIQ (“PEEML QAIQ” disclosed | | |
| | as SEQ ID NO: 45) d275-282 N287TMFFA (“TMFFA” disclosed | | |
| | as SEQ ID NO: 54) d288-291 d295 L298IM T300A I304E I309L | | |
| | P310L A314SHVRSV (“SHVRSV” disclosed as SEQ ID NO: 56) | | |
| | d315-319 A328RELE (“RELE” disclosed as SEQ ID NO: 58) | | |
| | d329-331 L335R T336K T339S N341T L351V K374P E375P | | |
| | I378P Q389E R391K N394D V398N L410NTENSPE | | |
| | (“NTENSPE” disclosed as SEQ ID NO: 84) d411-416 L419G | | |
| | D433K H436W E443RASFA (“RASFA” disclosed as SEQ ID | | |
| | NO: 66) d444-447 D455G Y459D R461G L471R K474EDGH | | |
| | (“EDGH” disclosed as SEQ ID NO: 67) d475-477 A508P Q509D | | |
| | T513A V521P D540T V542E T546K Q551K d562 I565F | | |
| | D566VE | | |
| 344 | T116W | 0.144 | − |
| 345 | N7T A8S D20W G30D S39A V40L K44H Q47R S50TQKEL | −0.046 | − |
| | (“TQKEL” disclosed as SEQ ID NO: 85) d51-54 K60R L64A | | |
| | Y69L C72T R73G E78V S82G M83R L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V1291 T131G A138S A140E V144T T149R Y152I | | |
| | A159G E162HFDAF (“HFDAF” disclosed as SEQ ID NO: 26) | | |
| | d163-166 A170G H189G R213M S221A E224H N227G E231Q | | |
| | S232D Q238DISAC (“DISAC” disclosed as SEQ ID NO: 34) | | |
| | d239-242 F255ILLANV (“ILLANV” disclosed as SEQ ID NO: | | |
| | 35) d256-260 Y268IMNR (“IMNR” disclosed as SEQ ID NO: 43) | | |
| | d269-271 I275EEML QAIQ (“EEML QAIQ” disclosed as SEQ ID | | |
| | NO: 46) d276-282 N287THFFA (“THFFA” disclosed as SEQ ID | | |
| | NO: 80) d288-291 d295 L298IM T300A I304E I309L P310L | | |
| | A314S M316V I319V A328RELE (“RELE” disclosed as SEQ ID | | |
| | NO: 58) d329-331 T336K T339S N341H L351V K374P E375P | | |
| | I378P Q389E N394D V398N D411TENAPR (“TENAPR” | | |
| | disclosed as SEQ ID NO: 63) d412-416 L419G Q430N D433K | | |
| | H436W C445S V447A D455G Y459D R461G L471R | | |
| | K474EDGH (“EDGH” disclosed as SEQ ID NO: 67) d475-477 | | |
| | G506RHPD (“RHPD” disclosed as SEQ ID NO: 69) d507-509 | | |
| | T513A V521P D540T V542E T546K Q551R d562 I565F | | |
| | D566VE | | |
| 346 | V212A | 0.531 | − |
| 347 | A324M | −0.088 | − |
| 348 | E372W | 0.566 | − |
| 349 | D566E | 0.653 | − |
| 350 | D373P | 0.008 | − |
| 351 | L198W | −0.034 | − |
| 352 | D370S | 0.364 | − |
| 353 | V390C | 1.198 | + |
| 354 | T220M | 0.003 | − |
| 355 | K254V | 0.334 | − |
| 356 | F255I | 0.195 | − |
| 357 | I565V | 1.49 | + |
| 358 | Q416R | 0.343 | − |
| 359 | Q416N | 1.18 | + |
| 360 | L298M | 0.67 | − |
| 361 | L320V | 0.066 | − |
| 362 | S230P | −0.06 | − |
| 363 | I275E | 1.038 | + |
| 364 | S230T | 0.368 | − |
| 365 | E162H | −0.021 | − |
| 366 | Y19F | 1.658 | + |
| 367 | E443R | 1.164 | + |
| 368 | M172L | 1.132 | + |
| 369 | I378V | 2.581 | ++ |
| 370 | L107V | 0.227 | − |
| 371 | N12E | 1.039 | + |
| 372 | M172A | 1.144 | + |
| 373 | A196I | −0.059 | − |
| 374 | N7T A8S D20W G30D S39A V40L K44T Q47RTYTQKEL | −0.058 | − |
| | (“RTYTQKEL” disclosed as SEQ ID NO: 15) d48-54 K60R L64A | | |
| | Y69L C72T R73G S82G M83R L87VAV d88-89 V94TIEAA | | |
| | (“TIEAA” disclosed as SEQ ID NO: 19) d95-98 I111VTPAN | | |
| | (“VTPAN” disclosed as SEQ ID NO: 20) d112-115 S119T Q121R | | |
| | V129H T131G A138S A140E V144T T149R Y152I A159G | | |
| | E162HFDSF (“HFDSF” disclosed as SEQ ID NO: 25) d163-166 | | |
| | A170G H189G R213M S221A E224H N227GKWSQN | | |
| | (“GKWSQN” disclosed as SEQ ID NO: 86) d228-232 Q238D | | |
| | A240S F255ILLANVHY (“ILLANVHY” disclosed as SEQ ID | | |
| | NO: 36) d256-262 Y268IMPR (“IMPR” disclosed as SEQ ID NO: | | |
| | 42) d269-271 I275EEMLQAIQ (“EEML QAIQ” disclosed as SEQ | | |
| | ID NO: 46) d276-282 N287TMFFA (“TMFFA” disclosed as SEQ | | |
| | ID NO: 54) d288-291 d295 L298LM T300A I304E I309L P310L | | |
| | A314SHVRSV (“SHVRSV” disclosed as SEQ ID NO: 56) d315- | | |
| | 319 N329ELE d330-331 L335R T336K T339S N341T L351V | | |
| | K374T E375P I378P P386A Q389E N394D V398N | | |
| | D411TENAPR (“TENAPR” disclosed as SEQ ID NO: 63) d412- | | |
| | 416 L419G D433K H436W E443RASFA (“RASFA” disclosed as | | |
| | SEQ ID NO: 66) d444-447 D455G Y459D R461G L471R | | |
| | K474EDGH (“EDGH” disclosed as SEQ ID NO: 67) d475-477 | | |
| | A508P Q509D T513A V521P D540T V542E T546K Q551R | | |
| | I562V I565V | | |
| 375 | D433K | 0.097 | − |
| 376 | D106S | −0.186 | − |
| 377 | H315S | 0.165 | − |
| 378 | L298A | 0.318 | − |
| 379 | L334W | −0.079 | − |
| 380 | S223I | 1.944 | + |
| 381 | A241P | −0.084 | − |
| 382 | V99C | 0.815 | − |
| 383 | M316V | 0.171 | − |
| 384 | N7T A8S Y19Q G30D S39A V40L K44H Q47R L49Y M54L | −0.078 | − |
| | K60R L64A Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V1291 T131G A138S A140E V144T T149R | | |
| | Y152A A159G E162HFDSF (“HFDSF” disclosed as SEQ ID NO: | | |
| | 25) d163-166 A170G H189G R213M T220M S221A | | |
| | N227GKWSQD (“GKWSQD” disclosed as SEQ ID NO: 28) | | |
| | d228-232 Q238D A240S F255I I257L P260V Y268IMNR | | |
| | (“IMNR” disclosed as SEQ ID NO: 43) d269-271 | | |
| | I275EEMLRAIQ (“EEMLRAIQ” disclosed as SEQ ID NO: 47) | | |
| | d276-282 N287THFFA (“THFFA” disclosed as SEQ ID NO: 80) | | |
| | d288-291 d295 L298IM T300A I304M I309L P310L A314S | | |
| | M316V I319V A328RELE (“RELE” disclosed as SEQ ID NO: 58) | | |
| | d329-331 L335R T336K T339S N341H L351V K374T E375P | | |
| | I378P P386A Q389E N394D V398N D411TENAPR (“TENAPR” | | |
| | disclosed as SEQ ID NO: 63) d412-416 L419G D433K H436W | | |
| | E443RASFA (“RASFA” disclosed as SEQ ID NO: 66) d444-447 | | |
| | D455G Y459D R461G L471R K474EDGF (“EDGF” disclosed as | | |
| | SEQ ID NO: 74) d475-477 A508P Q509D T513A V521P D540T | | |
| | V542E T546K Q551R d562 I565F D566VE | | |
| 385 | C222S | 0.566 | − |
| 386 | Q430N | −0.092 | − |
| 387 | R352E | 0.67 | − |
| 388 | N7T A8S D20W G30D S39A V40L K44H Q47R L49Y M54L | 0.019 | − |
| | K60R Y61F L64A Y69L R73G D76T E78V S82A M83K L87VA | | |
| | d89 V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129I T131G V136L YSKE (“LYSKE” disclosed | | |
| | as SEQ ID NO: 87) d137-140 V144T T149R Y152I A159G | | |
| | E162HFDSF (“HFDSF” disclosed as SEQ ID NO: 25) d163-166 | | |
| | A170G H189G R213M S221A E224H N227G E231QDLK | | |
| | (“QDLK” disclosed as SEQ ID NO: 88) d232-234 Q238D A240S | | |
| | F255ILLSNVHY (“ILLSNVHY” disclosed as SEQ ID NO: 41) | | |
| | d256-262 Y268IMNR (“IMNR” disclosed as SEQ ID NO: 43) | | |
| | d269-271 I275K V277M A280AI d282 N287TMFHA (“TMFHA” | | |
| | disclosed as SEQ ID NO: 71) d288-291 d295 L298IM T300A | | |
| | I304E I309L P310L A314SHVRSV (“SHVRSV” disclosed as | | |
| | SEQ ID NO: 56) d315-319 N329ELE d330-331 L335R T336K | | |
| | T339S N341T L351V K374T E375P I378P P386A Q389E R391K | | |
| | N394D V398N A402L D411TENAPR (“TENAPR” disclosed as | | |
| | SEQ ID NO: 63) d412-416 L419G Q430N D433K H436W | | |
| | E443RASFA (“RASFA” disclosed as SEQ ID NO: 66) d444-447 | | |
| | D455G Y459D R461G L471K K474EDGY (“EDGY” disclosed as | | |
| | SEQ ID NO: 68) d475-477 G506RHPD (“RHPD” disclosed as | | |
| | SEQ ID NO: 69) d507-509 T513A V521P D540T V542D T546A | | |
| | Q551R d562 I565F D566VE | | |
| 389 | K474E | 0.204 | − |
| 390 | S115N | 1.35 | + |
| 391 | N525T | 2.155 | ++ |
| 392 | V94S | 0.719 | − |
| 393 | L166F | 1.365 | + |
| 394 | D106Y | 0.002 | − |
| 395 | V354Y | 0.642 | − |
| 396 | S221T | 1.181 | + |
| 397 | Y61F | 0.95 | − |
| 398 | T367A | 0.083 | − |
| 399 | K282Q | 0.894 | − |
| 400 | A165S | 1.211 | + |
| 401 | N7K A8S Y19Q G30D S39A V40L A43G K44T Q47R L49Y | −0.075 | − |
| | A52K M54L K60R L64A S66R Y69L R73G E78V S82N M83K | | |
| | L87VA d89 V94TIEAA (“TIEAA” disclosed as SEQ ID NO: 19) | | |
| | d95-98 I111VTPAN (“VTPAN” disclosed as SEQ ID NO: 20) | | |
| | d112-115 S119T Q121R V129H T131G A138S A140E V144T | | |
| | T149R Y152I A159G E162RIESF (“RIESF” disclosed as SEQ ID | | |
| | NO: 24) d163-166 A170G R182Q H189G R213M T220M S221A | | |
| | N227GKWSQD (“GKWSQD” disclosed as SEQ ID NO: 28) | | |
| | d228-232 Q238D A240S F255ILLANVHY (“ILLANVHY” | | |
| | disclosed as SEQ ID NO: 36) d256-262 Y268IMNR (“IMNR” | | |
| | disclosed as SEQ ID NO: 43) d269-271 I275EEMLQAIQ | | |
| | (“EEMLQAIQ” disclosed as SEQ ID NO: 46) d276-282 | | |
| | N287THFFA (“THFFA” disclosed as SEQ ID NO: 80) d288-291 | | |
| | d295 L298IM T300A I309L P310L A314SHVRSV (“SHVRSV” | | |
| | disclosed as SEQ ID NO: 56) d315-319 A328RELE (“RELE” | | |
| | disclosed as SEQ ID NO: 58) d329-331 L335RKRMSGT | | |
| | (“RKRMSGT” disclosed as SEQ ID NO: 77) d336-341 L351V | | |
| | K374T I378P Q389E N394D V398N A402L D411TENAPR | | |
| | (“TENAPR” disclosed as SEQ ID NO: 63) d412-416 L419G | | |
| | D433K H436W E443RASFA (“RASFA” disclosed as SEQ ID | | |
| | NO: 66) d444-447 D455S Y459D R461G L471R K474EDGH | | |
| | (“EDGH” disclosed as SEQ ID NO: 67) d475-477 G506RHPD | | |
| | (“RHPD” disclosed as SEQ ID NO: 69) d507-509 T513A V521P | | |
| | D540T V542D T546A Q551R d562 I565FV | | |
| 402 | V129I | 1.543 | + |
| 403 | L424V | 0.032 | − |
| 404 | N7T A8S Y19Q S39A V40L K44H Q47R L49Y M54L K60R | 0 | − |
| | L64A Y69L C72T R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129I T131G A138S A140E V144T T149R | | |
| | Y152A A159G E162HFDSF (“HFDSF” disclosed as SEQ ID NO: | | |
| | 25) d163-166 A170G H189G R213M T220M S221A N227G | | |
| | E231QDLK (“QDLK” disclosed as SEQ ID NO: 88) d232-234 | | |
| | Q238DISAC (“DISAC” disclosed as SEQ ID NO: 34) d239-242 | | |
| | F255ILLSNV (“ILLSNV” disclosed as SEQ ID NO: 89) d256-260 | | |
| | Y268IMNR (“IMNR” disclosed as SEQ ID NO: 43) d269-271 | | |
| | I275EEML QAIQ (“EEML QAIQ” disclosed as SEQ ID NO: 46) | | |
| | d276-282 N287TMFHA (“TMFHA” disclosed as SEQ ID NO: 71) | | |
| | d288-291 d295 L298IM T300A I304R I309L P310L | | |
| | A314SHVRSV (“SHVRSV” disclosed as SEQ ID NO: 56) d315- | | |
| | 319 A328RELE (“RELE” disclosed as SEQ ID NO: 58) d329-331 | | |
| | L335R T336K T339S N341T L351V K374T E375P I378P Q389E | | |
| | N394D V398N A402L D411TENAPR (“TENAPR” disclosed as | | |
| | SEQ ID NO: 63) d412-416 L419G D433K H436W E443KASFA | | |
| | (“KASFA” disclosed as SEQ ID NO: 82) d444-447 D455G | | |
| | Y459D R461G L471K K474EDGY (“EDGY” disclosed as SEQ | | |
| | ID NO: 68) d475-477 G506RHPD (“RHPD” disclosed as SEQ ID | | |
| | NO: 69) d507-509 T513A V521P D540T V542E T546K Q551R | | |
| | I562V I565V | | |
| 405 | L274P | 0.023 | − |
| 406 | V297A | 0.064 | − |
| 407 | N7T A8S Y19Q G30D S39A V40L K44H Q47R L49Y M54L | −0.155 | − |
| | K60R L64G Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152T A159G E162RIESF (“RIESF” disclosed as SEQ ID NO: | | |
| | 24) d163-166 A170G H189G R213M T220M S221A | | |
| | N227GKWSQQ (“GKWSQQ” disclosed as SEQ ID NO: 90) | | |
| | d228-232 Q238D A240S F255I I257L P260V Y268IMNR | | |
| | (“IMNR” disclosed as SEQ ID NO: 43) d269-271 | | |
| | I275EEML QAIQ (“EEML QAIQ” disclosed as SEQ ID NO: 46) | | |
| | d276-282 N287TFFFA (“TFFFA” disclosed as SEQ ID NO: 53) | | |
| | d288-291 d295 L298IM T300A I304M I309L P310L A314S | | |
| | I319V A328RELE (“RELE” disclosed as SEQ ID NO: 58) d329- | | |
| | 331 T336K T339S N341T E348D L351V K374P E375P I378P | | |
| | Q389E N394D V398N A402L D411TENAPR (“TENAPR” | | |
| | disclosed as SEQ ID NO: 63) d412-416 L419G D433K H436W | | |
| | E443RASFA (“RASFA” disclosed as SEQ ID NO: 66) d444-447 | | |
| | D455G Y459D R461G L471R K474EDGH (“EDGH” disclosed as | | |
| | SEQ ID NO: 67) d475-477 G506RHPD (“RHPD” disclosed as | | |
| | SEQ ID NO: 69) d507-509 T513A V521P D540T V542D T546A | | |
| | Q551R d562 I565FV | | |
| 408 | K254L | 0.41 | − |
| 409 | F91M | 0.199 | − |
| 410 | M172D | 1.191 | + |
| 411 | G251H | −0.069 | − |
| 412 | I239V | 0.859 | − |
| 413 | I275R | 0.748 | − |
| 414 | S230E | −0.002 | − |
| 415 | N153G | 0.959 | − |
| 416 | L233R | 0.52 | − |
| 417 | R73V | 0.915 | − |
| 418 | I4M N7T A8S D20W G30D S39A V40L K44S Q47R L49Y | −0.096 | − |
| | M54L K60R L64A Y69L C72T R73G E78V S82N M83K L87V | | |
| | T89V V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152I A159G E162HFDAF (“HFDAF” disclosed as SEQ ID NO: | | |
| | 26) d163-166 A170G H189G R213M S221A E224H | | |
| | N227GKWSQD (“GKWSQD” disclosed as SEQ ID NO: 28) | | |
| | d228-232 Q238DISAC (“DISAC” disclosed as SEQ ID NO: 34) | | |
| | d239-242 F255ILLANV (“ILLANV” disclosed as SEQ ID NO: | | |
| | 35) d256-260 Y268IMPR (“IMPR” disclosed as SEQ ID NO: 42) | | |
| | d269-271 I275K V277M A280AI d282 N287TVFFA (“TVFFA” | | |
| | disclosed as SEQ ID NO: 51) d288-291 d295 L298IM T300A | | |
| | I304E I309L P310L A314SHVRSV (“SHVRSV” disclosed as | | |
| | SEQ ID NO: 56) d315-319 N329ELE d330-331 L335RKRMSGT | | |
| | (“RKRMSGT” disclosed as SEQ ID NO: 77) d336-341 L351V | | |
| | K374T I378P P386A Q389E R391K N394D V398N | | |
| | L410NTENAPE (“NTENAPE” disclosed as SEQ ID NO: 91) | | |
| | d411-416 L419G D433K H436W E443RASFA (“RASFA” | | |
| | disclosed as SEQ ID NO: 66) d444-447 D455G Y459D R461G | | |
| | L471K K474EDGY (“EDGY” disclosed as SEQ ID NO: 68) | | |
| | d475-477 G506RHPD (“RHPD” disclosed as SEQ ID NO: 69) | | |
| | d507-509 T513A V521P D540T V542E T546K Q551R d562 | | |
| | I565FV | | |
| 419 | A159G | 0.591 | − |
| 420 | S253I | 0.71 | − |
| 421 | D16E | 1.06 | + |
| 422 | G199M | 0.724 | − |
| 423 | D455G | 1.048 | + |
| 424 | A264W | 0.074 | − |
| 425 | A109V | 0.169 | − |
| 426 | I353L | 1.85 | + |
| 427 | H248P | −0.079 | − |
| 428 | T131G | 1.054 | + |
| 429 | L320Y | 3.664 | ++ |
| 430 | L233M | 0.534 | − |
| 431 | S202E | −0.077 | − |
| 432 | R317Q | −0.05 | − |
| 433 | K554S | 2.718 | ++ |
| 434 | L64A | 1.263 | + |
| 435 | M83R | −0.056 | − |
| 436 | V469M | 2.587 | ++ |
| 437 | Y268F | 1.046 | + |
| 438 | Y478I | 0.162 | − |
| 439 | L49Y | 0.54 | − |
| 440 | S112T | −0.077 | − |
| 441 | V390L | 0.001 | − |
| 442 | K383Y | 0.624 | − |
| 443 | Q121R | 0.732 | − |
| 444 | V542D | 0.077 | − |
| 445 | T290V | 1.097 | + |
| 446 | V246W | 0.024 | − |
| 447 | T266V | 0.343 | − |
| 448 | I304M | 0.453 | − |
| 449 | R213E | 0.041 | − |
| 450 | D406L | 1.363 | + |
| 451 | W55L | −0.028 | − |
| 452 | D106R | −0.051 | − |
| 453 | D370H | 0.254 | − |
| 454 | Q238H | 1.474 | + |
| 455 | Q51R | −0.185 | − |
| 456 | T331E | −0.022 | − |
| 457 | G356A | 0.818 | − |
| 458 | A229L | 1.821 | + |
| 459 | Y19P | −0.028 | − |
| 460 | I426L | 0.764 | − |
| 461 | N227E | 1.121 | + |
| 462 | M479H | 0.11 | − |
| 463 | S232Y | 1.19 | + |
| 464 | W289V | 0.085 | − |
| 465 | Y152T | 0.527 | − |
| 466 | A262Y | 0.202 | − |
| 467 | I319F | 0.049 | − |
| 468 | F127M | 0.824 | − |
| 469 | R477E | −0.125 | − |
| 470 | W425V | −0.15 | − |
| 471 | S288R | 0.308 | − |
| 472 | G199L | 0.011 | − |
| 473 | A328V | 1.285 | + |
| 474 | F369Y | 0.69 | − |
| 475 | N7T A8S D20W G30D S39A V40L K44H Q47R L49Y M54L | −0.066 | − |
| | K60R L64A Y69L R73G E78V S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152I A159G E162R L163I L166F A170G H189G R213M | | |
| | S221A E224H N227GKWSQD (“GKWSQD” disclosed as SEQ | | |
| | ID NO: 28) d228-232 Q238DISAC (“DISAC” disclosed as SEQ | | |
| | ID NO: 34) d239-242 F255ILLANV (“ILLANV” disclosed as | | |
| | SEQ ID NO: 35) d256-260 Y268IMNR (“IMNR” disclosed as | | |
| | SEQ ID NO: 43) d269-271 I275EEMLQAIQ (“EEML QAIQ” | | |
| | disclosed as SEQ ID NO: 46) d276-282 N287TVFFA (“TVFFA” | | |
| | disclosed as SEQ ID NO: 51) d288-291 d295 L298IM T300A | | |
| | I304E I309L P310L A314SHVRSV (“SHVRSV” disclosed as | | |
| | SEQ ID NO: 56) d315-319 N329ELE d330-331 L335R T336K | | |
| | T339S N341T L351V K374T I378P P386A Q389E N394D | | |
| | V398N D411TENAPR (“TENAPR” disclosed as SEQ ID NO: 63) | | |
| | d412-416 L419G D433K H436W E443RASFA (“RASFA” | | |
| | disclosed as SEQ ID NO: 66) d444-447 D455G Y459D R461G | | |
| | L471K K474EDGY (“EDGY” disclosed as SEQ ID NO: 68) | | |
| | d475-477 G506RHPD (“RHPD” disclosed as SEQ ID NO: 69) | | |
| | d507-509 T513A V521P D540T V542E T546K Q551K d562 | | |
| | I565F D566VE | | |
| 476 | A508P | 1.457 | + |
| 477 | F90L | 1.097 | + |
| 478 | C72T | −0.13 | − |
| 479 | V267Y | 0.375 | − |
| 480 | L198I | 0.654 | − |
| 481 | S201T | 0.966 | − |
| 482 | S209P | 1.194 | + |
| 483 | K523A | 1.236 | + |
| 484 | E224H | −0.167 | − |
| 485 | I414A | 0.406 | − |
| 486 | S253H | 1.153 | + |
| 487 | T89V | 0.249 | − |
| 488 | S363T | −0.052 | − |
| 489 | M316L | 1.188 | + |
| 490 | R182Q | 1.47 | + |
| 491 | L384P | −0.098 | − |
| 492 | S288I | 1.34 | − |
| 493 | Q126H | 0.271 | − |
| 494 | Y61L | 0.25 | − |
| 495 | S388V | 0.11 | − |
| 496 | N7T A8S Y19Q G30D S39A V40L K44H Q47R L49Y M54L | −0.076 | − |
| | K60R L64A Y69L C72T R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D F127MRHTG (“MRHTG” disclosed as SEQ ID | | |
| | NO: 21) d128-131 A138S A140E V144T T149R Y152A A159G | | |
| | E162HFDSF (“HFDSF” disclosed as SEQ ID NO: 25) d163-166 | | |
| | A170G H189G R213M T220M S221A N227G E231QDLQ | | |
| | (“QDLQ” disclosed as SEQ ID NO: 33) d232-234 Q238DISAC | | |
| | (“DISAC” disclosed as SEQ ID NO: 34) d239-242 | | |
| | F255ILLANVHY (“ILLANVHY” disclosed as SEQ ID NO: 36) | | |
| | d256-262 Y268IMNR (“IMNR” disclosed as SEQ ID NO: 43) | | |
| | d269-271 I275EEMLQAIQ (“EEML QAIQ” disclosed as SEQ ID | | |
| | NO: 46) d276-282 N287TSFFA (“TSFFA” disclosed as SEQ ID | | |
| | NO: 52) d288-291 d295 L298IM T300A I304E I309L P310L | | |
| | A314S A318T I319V A328RELE (“RELE” disclosed as SEQ ID | | |
| | NO: 58) d329-331 L335Q T336K T339S N341T E348D L351V | | |
| | K374Q E375P I378P P386A Q389E N394D V398N | | |
| | D411TQNAPD (“TQNAPD” disclosed as SEQ ID NO: 92) d412- | | |
| | 416 L419G D433K H436W E443RASFA (“RASFA” disclosed as | | |
| | SEQ ID NO: 66) d444-447 D455G Y459D R461G L471R | | |
| | K474EDGY (“EDGY” disclosed as SEQ ID NO: 68) d475-477 | | |
| | A508P Q509D T513A V521P D540T V542E T546K Q551R d562 | | |
| | I565F D566VE | | |
| 497 | L334C | 0.363 | − |
| 498 | N7K | 1.594 | + |
| 499 | N7T A8S Y19Q G30D T36H S39A V40L K44H Q47RTYTQREL | −0.015 | − |
| | (“RTYTQREL” disclosed as SEQ ID NO: 16) d48-54 K60R L64A | | |
| | Y69L R73G S82G M83K L87VAV d88-89 V94TIEAA (“TIEAA” | | |
| | disclosed as SEQ ID NO: 19) d95-98 I111VTPAN (“VTPAN” | | |
| | disclosed as SEQ ID NO: 20) d112-115 S119T Q121R V129I | | |
| | T131G A138S A140E V144T T149R Y152T A159G E162HFDSF | | |
| | (“HFDSF” disclosed as SEQ ID NO: 25) d163-166 A170G H189G | | |
| | R213M T220M S221A N227G E231Q S232D Q238D A240S | | |
| | F255I I257L P260V Y268IMNR (“IMNR” disclosed as SEQ ID | | |
| | NO: 43) d269-271 I275EEMLQAIQ (“EEMLQAIQ” disclosed as | | |
| | SEQ ID NO: 46) d276-282 N287THMFA (“THMFA” disclosed as | | |
| | SEQ ID NO: 93) d288-291 d295 L298IM T300A I304R I309L | | |
| | P310L A314S I319V A328RELE (“RELE” disclosed as SEQ ID | | |
| | NO: 58) d329-331 L335R T336K T339S N341T L351V K374T | | |
| | E375P I378P Q389E N394D V398N A402L D411TENAPD | | |
| | (“TENAPD” disclosed as SEQ ID NO: 61) d412-416 L419G | | |
| | D433K H436W E443RASFA (“RASFA” disclosed as SEQ ID | | |
| | NO: 66) d444-447 D455G Y459D R461G L471R K474EDGH | | |
| | (“EDGH” disclosed as SEQ ID NO: 67) d475-477 A508P Q509D | | |
| | T513A V521P D540T V542D T546A Q551R I562V I565V | | |
| 500 | L320T | 1.438 | + |
| 501 | D80G | 0.243 | − |
| 502 | V246F | 0.254 | − |
| 503 | W289A | −0.229 | − |
| 504 | R352K | 1.871 | + |
| 505 | F369I | 0.768 | − |
| 506 | L335Q | 0.108 | − |
| 507 | L326V | −0.155 | − |
| 508 | A496S | 0.98 | − |
| 509 | N227M | 0.933 | − |
| 510 | W425L | −0.006 | − |
| 511 | V354A | 0.158 | − |
| 512 | A140K | 1.057 | + |
| 513 | K374T | 1.101 | + |
| 514 | K150L | 1.14 | + |
| 515 | K374A | 1.517 | + |
| 516 | V354K | −0.047 | − |
| 517 | L320M | 1.098 | + |
| 518 | Y459G | −0.089 | − |
| 519 | E279R | 0.138 | − |
| 520 | N7T A8S Y19F D20W G30D S39A V40L K44H Q47R L49Y | −0.194 | − |
| | M54L K60R L64A Y69L C72T R73G S82N M83K L87VAV | | |
| | d88-89 V94TIEAA (“TIEAA” disclosed as SEQ ID NO: 19) d95- | | |
| | 98 I111VTPAN (“VTPAN” disclosed as SEQ ID NO: 20) d112- | | |
| | 115 S119T Q121D V129H T131G A138S A140E V144T T149R | | |
| | Y152C N153S A159G E162HFDSF (“HFDSF” disclosed as SEQ | | |
| | ID NO: 25) d163-166 A170G H189G R213M S221A | | |
| | N227GKWSQD (“GKWSQD” disclosed as SEQ ID NO: 28) | | |
| | d228-232 Q238D A240S F255ILLANV (“ILLANV” disclosed as | | |
| | SEQ ID NO: 35) d256-260 Y268IMNR (“IMNR” disclosed as | | |
| | SEQ ID NO: 43) d269-271 I275K V277M A280AI d282 | | |
| | N287TVFFA (“TVFFA” disclosed as SEQ ID NO: 51) d288-291 | | |
| | d295 L298IM T300A I304M I309L P310L A314SHVRTV | | |
| | (“SHVRTV” disclosed as SEQ ID NO: 57) d315-319 A328RELE | | |
| | (“RELE” disclosed as SEQ ID NO: 58) d329-331 L335R T336K | | |
| | T339S N341H L351V K374T E375P I378P Q389E N394D | | |
| | V398N D411TQNAPD (“TQNAPD” disclosed as SEQ ID NO: | | |
| | 92) d412-416 L419G D433K H436W C445S V447A D455G | | |
| | Y459D R461G L471K K474EDGY (“EDGY” disclosed as SEQ | | |
| | ID NO: 68) d475-477 G506RHPD (“RHPD” disclosed as SEQ ID | | |
| | NO: 69) d507-509 T513A V521P D540T V542E T546K Q551R | | |
| | I562V I565V | | |
| 521 | P407D | 1.956 | + |
| 522 | A229G | 0.141 | − |
| 523 | V100S | −0.094 | − |
| 524 | N7T A8S Y19Q G30D S39A V40L K44H Q47R L49Y M54L | −0.092 | − |
| | K60R L64A Y69L R73G E78V S82N M83K L87VA d89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152T A159G E162R L1631 L I66F A170G H189G R213M | | |
| | T220M S221A N227GKWSQD (“GKWSQD” disclosed as SEQ | | |
| | ID NO: 28) d228-232 Q238D A240S F255ILLANV (“ILLANV” | | |
| | disclosed as SEQ ID NO: 35) d256-260 Y268IMNR (“IMNR” | | |
| | disclosed as SEQ ID NO: 43) d269-271 I275EEMLQAIQ | | |
| | (“EEMLQAIQ” disclosed as SEQ ID NO: 46) d276-282 | | |
| | N287TVFFA (“TVFFA” disclosed as SEQ ID NO: 51) d288-291 | | |
| | d295 L298IM T300A I304E I309L P310L A314SHVRSV | | |
| | (“SHVRSV” disclosed as SEQ ID NO: 56) d315-319 A328RELE | | |
| | (“RELE” disclosed as SEQ ID NO: 58) d329-331 T336K T339S | | |
| | N341TL351V K374P E375P I378P P386A Q389E N394D | | |
| | V398N D411TENAPR (“TENAPR” disclosed as SEQ ID NO: 63) | | |
| | d412-416 L419G D433K H436W C445S V447A D455G Y459D | | |
| | R461G L471R K474EDGH (“EDGH” disclosed as SEQ ID NO: | | |
| | 67) d475-477 G506RHPD (“RHPD” disclosed as SEQ ID NO: 69) | | |
| | d507-509 T513A V521P D540T V542D T546A Q551R d562 | | |
| | I565F D566VE | | |
| 525 | R529V | −0.08 | − |
| 526 | H261A | 0.354 | − |
| 527 | N7T A8S D20W G30D S39A V40L K44H Q47R L49Y M54L | −0.072 | − |
| | K60R L64A Y69L R73G S82A M83K L87V T89V V94TIEAA | | |
| | (“TIEAA” disclosed as SEQ ID NO: 19) d95-98 I111VTPAN | | |
| | (“VTPAN” disclosed as SEQ ID NO: 20) d112-115 S119T Q121R | | |
| | V129I T131G A138S A140E V144T T149R Y152I A159G | | |
| | E162HFDSF (“HFDSF” disclosed as SEQ ID NO: 25) d163-166 | | |
| | A170G H189G R213M S221A N227G E231Q A234Q | | |
| | Q238DISAC (“DISAC” disclosed as SEQ ID NO: 34) d239-242 | | |
| | F255ILLANVHY (“ILLANVHY” disclosed as SEQ ID NO: 36) | | |
| | d256-262 Y268IMNR (“IMNR” disclosed as SEQ ID NO: 43) | | |
| | d269-271 I275EEML QAIQ (“EEMLQAIQ” disclosed as SEQ ID | | |
| | NO: 46) d276-282 N287TVFFA (“TVFFA” disclosed as SEQ ID | | |
| | NO: 51) d288-291 d295 L298IM T300A I304M I309L P310L | | |
| | A314SHVRSV (“SHVRSV” disclosed as SEQ ID NO: 56) d315- | | |
| | 319 A328RELE (“RELE” disclosed as SEQ ID NO: 58) d329-331 | | |
| | L335QKRMSGT (“QKRMSGT” disclosed as SEQ ID NO: 94) | | |
| | d336-341 E348D L351V K374T E375P I378P Q389E N394D | | |
| | V398N A402L D411TENAPE (“TENAPE” disclosed as SEQ ID | | |
| | NO: 73) d412-416 L419G D433K H436W E443RASFA | | |
| | (“RASFA” disclosed as SEQ ID NO: 66) d444-447 D455G Y459E | | |
| | R461G L471R K474EDGY (“EDGY” disclosed as SEQ ID NO: | | |
| | 68) d475-477 A508P Q509D T513A V521P D540T V542E | | |
| | T546K Q551R I562V I565V D566E | | |
| 528 | Y539L | 0.072 | − |
| 529 | V136L | 0.155 | − |
| 530 | N7T A8S Y19F G30D S39A V40L K44H Q47R L49Y M54L | −0.299 | − |
| | K60R L64A Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140E V144T T149R | | |
| | Y152C A159G E162HFDSF (“HFDSF” disclosed as SEQ ID NO: | | |
| | 25) d163-166 A170G H189G R213M S221A E224H | | |
| | N227GKWSQ (“GKWSQ” disclosed as SEQ ID NO: 30) d228- | | |
| | 231 Q238DISAC (“DISAC” disclosed as SEQ ID NO: 34) d239- | | |
| | 242 F255ILLLNV (“ILLLNV” disclosed as SEQ ID NO: 38) | | |
| | d256-260 Y268IMNR (“IMNR” disclosed as SEQ ID NO: 43) | | |
| | d269-271 I275EEMLQAIQ (“EEML QAIQ” disclosed as SEQ ID | | |
| | NO: 46) d276-282 N287TVFFA (“TVFFA” disclosed as SEQ ID | | |
| | NO: 51) d288-291 d295 L298IM T300A I304E I309L P310L | | |
| | A314SHVRTV (“SHVRTV” disclosed as SEQ ID NO: 57) d315- | | |
| | 319 A328RELE (“RELE” disclosed as SEQ ID NO: 58) d329-331 | | |
| | L335R T336K T339S N341T L351V K374P E375P I378P P386A | | |
| | Q389E N394D V398N A402L D411TENAPR (“TENAPR” | | |
| | disclosed as SEQ ID NO: 63) d412-416 L419G D433K H436W | | |
| | E443RASFA (“RASFA” disclosed as SEQ ID NO: 66) d444-447 | | |
| | D455G Y459D R461G L471R K474EDGH (“EDGH” disclosed as | | |
| | SEQ ID NO: 67) d475-477 A508P Q509D T513A V521P D540T | | |
| | V542D T546A Q551R d562 I565FV | | |
| 531 | N7T A8S Y19Q S39A V40L A43G K44S Q47R M54L K60R | −0.013 | − |
| | L64A Y69L R73G E78V S82N M83K L87V T89V V94TIEAA | | |
| | (“TIEAA” disclosed as SEQ ID NO: 19) d95-98 I111VTPAN | | |
| | (“VTPAN” disclosed as SEQ ID NO: 20) d112-115 S119T Q121R | | |
| | V129H T131G A138S A140E V144T T149R Y152C A159G | | |
| | E162HFDSF (“HFDSF” disclosed as SEQ ID NO: 25) d163-166 | | |
| | A170G H189G R213M T220M S221A N227G E231Q S232D | | |
| | Q238DISAC (“DISAC” disclosed as SEQ ID NO: 34) d239-242 | | |
| | F255ILLANV (“ILLANV” disclosed as SEQ ID NO: 35) d256- | | |
| | 260 Y268IMPR (“IMPR” disclosed as SEQ ID NO: 42) d269-271 | | |
| | L274PEEMLQAIQ (“PEEMLQAIQ” disclosed as SEQ ID NO: | | |
| | 45) d275-282 N287TVFFA (“TVFFA” disclosed as SEQ ID NO: | | |
| | 51) d288-291 d295 L298IM T300A I304E I309L P310L | | |
| | A314SHVRTV (“SHVRTV” disclosed as SEQ ID NO: 57) d315- | | |
| | 319 A328RELE (“RELE” disclosed as SEQ ID NO: 58) d329-331 | | |
| | T336K T339S N341H L351V K374T E375P I378P Q389E R391K | | |
| | N394D V398N A402L D411TENAPR (“TENAPR” disclosed as | | |
| | SEQ ID NO: 63) d412-416 L419G D433K H436W E443RASFA | | |
| | (“RASFA” disclosed as SEQ ID NO: 66) d444-447 D455G Y459E | | |
| | R461G L471K K474EDGY (“EDGY” disclosed as SEQ ID NO: | | |
| | 68) d475-477 A508P Q509D T513A V521P D540T V542E | | |
| | T546K Q551R I562V I565V D566E | | |
| 532 | Q430S | −0.058 | − |
| 533 | E372Q | 0.681 | − |
| 534 | L252M | 0.486 | − |
| 535 | E78V | 0.685 | − |
| 536 | L256C | 0.338 | − |
| 537 | V94T | 1.067 | + |
| 538 | V40F | 0.08 | − |
| 539 | V15L | 0.81 | − |
| 540 | V365M | 2.465 | ++ |
| 541 | D76T | 0.229 | − |
| 542 | F91S | −0.117 | − |
| 543 | I10F | 2.804 | ++ |
| 544 | K254P | −0.069 | − |
| 545 | T362C | 0.91 | − |
| 546 | A413N | 0.475 | − |
| 547 | T290P | 0.787 | − |
| 548 | K383W | 0.039 | − |
| 549 | H436W | 0.216 | − |
| 550 | K523R | 1.018 | + |
| 551 | S201N | 0.085 | − |
| 552 | M432T | 0.073 | − |
| 553 | A560G | 2.035 | ++ |
| 554 | Q281M | 0.19 | − |
| 555 | T300A | 2.118 | ++ |
| 556 | I295L | 0.234 | − |
| 557 | G199W | 0.15 | − |
| 558 | S202L | 0.034 | − |
| 559 | L291M | 0.067 | − |
| 560 | N7K A8S Y19Q G30D S39A V40L K44S Q47R L49Y M54L | −0.034 | − |
| | K60R L64A Y69L C72T R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152A A159G E162HFDSF (“HFDSF” disclosed as SEQ ID NO: | | |
| | 25) d163-166 A170G H189G R213M T220M S221A N227G | | |
| | E231Q S232D Q238DISAC (“DISAC” disclosed as SEQ ID NO: | | |
| | 34) d239-242 F255ILLANVHY (“ILLANVHY” disclosed as SEQ | | |
| | ID NO: 36) d256-262 Y268IMPR (“IMPR” disclosed as SEQ ID | | |
| | NO: 42) d269-271 I275K V277M A280AI d282 N287THFFA | | |
| | (“THFFA” disclosed as SEQ ID NO: 80) d288-291 d295 L298IM | | |
| | T300A E306D I309L P310L A314SHVRSV (“SHVRSV” | | |
| | disclosed as SEQ ID NO: 56) d315-319 N329ELE d330-331 | | |
| | L335R T336K T339S N341T L351V K374P E375P I378P P386A | | |
| | Q389E R391K N394D V398N D411TENAPR (“TENAPR” | | |
| | disclosed as SEQ ID NO: 63) d412-416 L419G D433K H436W | | |
| | E443RASFA (“RASFA” disclosed as SEQ ID NO: 66) d444-447 | | |
| | D455G Y459D R461G L471R K474EDGH (“EDGH” disclosed as | | |
| | SEQ ID NO: 67) d475-477 A508P Q509D T513A V521P D540T | | |
| | V542D T546A Q551R d562 I565F D566VE | | |
| 561 | R376E | 0.066 | − |
| 562 | K554P | 2.249 | ++ |
| 563 | A240T | 1.021 | + |
| 564 | V431L | 0.038 | − |
| 565 | N7K A8S Y19Q G30D S39A V40L A43G K44S Q47R | −0.09 | − |
| | S50TQKEL (“TQKEL” disclosed as SEQ ID NO: 85) d51-54 | | |
| | K60R L64A Y69L C72T R73G S82N M83K L87V T89V | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140E V144T T149R | | |
| | Y152A A159G E162HFDSF (“HFDSF” disclosed as SEQ ID NO: | | |
| | 25) d163-166 A170G H189G R213M T220M S221A | | |
| | N227GKWSQD (“GKWSQD” disclosed as SEQ ID NO: 28) | | |
| | d228-232 Q238D A240S F255I I257L P260V Y268IMPR | | |
| | (“IMPR” disclosed as SEQ ID NO: 42) d269-271 | | |
| | I275EEML QAIQ (“EEML QAIQ” disclosed as SEQ ID NO: 46) | | |
| | d276-282 N287THFFA (“THFFA” disclosed as SEQ ID NO: 80) | | |
| | d288-291 d295 L298IM T300A I304E I309L P310L A314S I319V | | |
| | A328RELE (“RELE” disclosed as SEQ ID NO: 58) d329-331 | | |
| | L335R T336K T339S N341T E348D L351V K374P E375P I378P | | |
| | P386A Q389E N394D V398N A402L D411TENAPR | | |
| | (“TENAPR” disclosed as SEQ ID NO: 63) d412-416 L419G | | |
| | D433K H436W E443RASFA (“RASFA” disclosed as SEQ ID | | |
| | NO: 66) d444-447 D455G Y459D R461G L471R K474EDGH | | |
| | (“EDGH” disclosed as SEQ ID NO: 67) d475-477 G506RHPD | | |
| | (“RHPD” disclosed as SEQ ID NO: 69) d507-509 T513A V521P | | |
| | D540T V542D T546A Q551R d562 I565F D566VE | | |
| 566 | L471M | 1.425 | + |
| 567 | S380T | 0.102 | − |
| 568 | T367C | 0.339 | − |
| 569 | I257L | 0.092 | − |
| 570 | I309L | 0.871 | − |
| 571 | M83P | −0.149 | − |
| 572 | N270P | 0.21 | − |
| 573 | N7T A8S Y19Q G30D T36H S39A V40L K44T Q47RTYT | 0.013 | − |
| | (“RTYT” disclosed as SEQ ID NO: 17)d48-50 M54L K60R L64A | | |
| | Y69L R73G S82G M83K L87VAV d88-89 V94TIEAA | | |
| | (“TIEAA” disclosed as SEQ ID NO: 19)d95-98 I111VTPAN | | |
| | (“VTPAN” disclosed as SEQ ID NO: 20)d112-115 S119T Q121D | | |
| | V129H T131G A138S A140E V144T T149R Y152I A159G | | |
| | E162HFDAF (“HFDAF” disclosed as SEQ ID NO: 26)d163-166 | | |
| | A170G H189G R213M T220M S221A N227G E231Q S232D | | |
| | Q238DVSAC (“DVSAC” disclosed as SEQ ID NO: 81) d239-242 | | |
| | F255I I257L P260V Y268IMNR (“IMNR” disclosed as SEQ ID | | |
| | NO: 43)d269-271 I275EEMLQAIQ (“EEML QAIQ” disclosed as | | |
| | SEQ ID NO: 46)d276-282 N287TVFFA (“TVFFA” disclosed as | | |
| | SEQ ID NO: 51)d288-291 d295 L298IM T300A I304E I309L | | |
| | P310L A314SHVRSV (“SHVRSV” disclosed as SEQ ID NO: | | |
| | 56)d315-319 N329ELE d330-331 L335R T336K T339S N341H | | |
| | L351V K374T I378P Q389E R391K N394D V398N A402L | | |
| | E412ENAPR (“ENAPR” disclosed as SEQ ID NO: 79) d413-416 | | |
| | L419G D433K H436W E443RASFA (“RASFA” disclosed as | | |
| | SEQ ID NO: 66)d444-447 D455G Y459D R461G L471K | | |
| | K474EDGH (“EDGH” disclosed as SEQ ID NO: 67)d475-477 | | |
| | G506RHPD (“RHPD” disclosed as SEQ ID NO: 69)d507-509 | | |
| | T513A V521P D540T V542D T546A Q551R d562 I565F | | |
| | D566VE | | |
| 574 | K342L | −0.003 | − |
| 575 | N329E | 0.581 | − |
| 576 | M432F | 1.375 | + |
| 577 | A229W | 0.151 | − |
| 578 | A262R | 0.873 | − |
| 579 | I304R | 0.822 | − |
| 580 | S490R | 0.183 | − |
| 581 | S82G | 0.267 | − |
| 582 | L252W | −0.003 | − |
| 583 | L351T | 0.269 | − |
| 584 | K44H | 0.85 | − |
| 585 | S226T | 0.677 | − |
| 586 | T265H | 0.019 | − |
| 587 | A242V | 0.053 | − |
| 588 | T367S | 0.199 | − |
| 589 | S201P | 0.005 | − |
| 590 | N341T | 0.006 | − |
| 591 | A240W | 0.287 | − |
| 592 | N7T | 0.427 | − |
| 593 | T331C | 0.539 | − |
| 594 | A170G | 0.026 | − |
| 595 | L471W | 1.873 | + |
| 596 | I353M | 0.203 | − |
| 597 | V100M | −0.004 | − |
| 598 | T331L | 0.278 | − |
| 599 | V277L | 1.693 | + |
| 600 | A318R | 1.758 | + |
| 601 | H261F | 0.95 | − |
| 602 | M479Y | 0.189 | − |
| 603 | E372H | 1.387 | + |
| 604 | Q51F | 0.046 | − |
| 605 | V246L | 0.645 | − |
| 606 | T116P | 0.844 | − |
| 607 | A264I | 0.598 | − |
| 608 | A262G | 0.77 | − |
| 609 | S217K | 0.864 | − |
| 610 | A262C | 1.609 | + |
| 611 | N525D | 1.583 | + |
| 612 | H377Y | 0.389 | − |
| 613 | E224I | 0.506 | − |
| 614 | N7T A8S Y19Q G30D T36H S39A V40L K44H Q47RTYT | −0.07 | − |
| | (“RTYT” disclosed as SEQ ID NO: 17) d48-50 M54L K60R | | |
| | L64A Y69L C72T R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140E V144T T149R | | |
| | Y152I A159G E162HFDSF (“HFDSF” disclosed as SEQ ID NO: | | |
| | 25) d163-166 A170G H189G R213M T220M S221A N227G | | |
| | E231Q S232D Q238D A240S F255I I257L P260V Y268IMNR | | |
| | (“IMNR” disclosed as SEQ ID NO: 43) d269-271 | | |
| | L274PEEMLRAIQ (“PEEMLRAIQ” disclosed as SEQ ID NO: | | |
| | 44) d275-282 N287THFFA (“THFFA” disclosed as SEQ ID NO: | | |
| | 80) d288-291 d295 L298IM T300A I304E I309L P310L | | |
| | A314SHVRSV (“SHVRSV” disclosed as SEQ ID NO: 56) d315- | | |
| | 319 A328RELE (“RELE” disclosed as SEQ ID NO: 58) d329-331 | | |
| | L335R T336K T339S N341T L351V K374T E375P I378P Q389E | | |
| | N394D V398N A402L D411TENAPR (“TENAPR” disclosed as | | |
| | SEQ ID NO: 63) d412-416 L419G D433K H436W E443RASFA | | |
| | (“RASFA” disclosed as SEQ ID NO: 66) d444-447 D455G | | |
| | Y459D R461G L471K K474EDGY (“EDGY” disclosed as SEQ | | |
| | ID NO: 68) d475-477 G506RHPD (“RHPD” disclosed as SEQ ID | | |
| | NO: 69) d507-509 T513A V521P D540T V542D T546A Q551R | | |
| | d562 I565F D566VE | | |
| 615 | S388L | −0.03 | − |
| 616 | V286P | −0.144 | − |
| 617 | P420E | 0.364 | − |
| 618 | A229I | 1.702 | + |
| 619 | K254A | 0.091 | − |
| 620 | S226W | 0.412 | − |
| 621 | I243L | 0.465 | − |
| 622 | V365C | 1.234 | + |
| 623 | K374R | 1.09 | + |
| 624 | I257T | −0.035 | − |
| 625 | V246Y | −0.045 | − |
| 626 | R73L | 1.19 | + |
| 627 | H559P | −0.077 | − |
| 628 | V472C | 0.438 | − |
| 629 | K374S | 1.171 | + |
| 630 | L368H | −0.02 | − |
| 631 | V94C | 0.602 | − |
| 632 | N7T A8S Y19Q G30D S39A V40L K44H Q47R L49Y M54L | −0.047 | − |
| | K60R L64A Y69L C72T R73G S82G M83R L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129I T131G A138S K139E V144T T149R Y152I | | |
| | A159G E162HFDAF (“HFDAF” disclosed as SEQ ID NO: 26) | | |
| | d163-166 A170G H189G R213M T220M S221A N227GKWSQ | | |
| | (“GKWSQ” disclosed as SEQ ID NO: 30) d228-231 Q238D | | |
| | A240S F255ILLLNV (“ILLLNV” disclosed as SEQ ID NO: 38) | | |
| | d256-260 Y268IMNR (“IMNR” disclosed as SEQ ID NO: 43) | | |
| | d269-271 I275EEML QAIQ (“EEML QAIQ” disclosed as SEQ ID | | |
| | NO: 46) d276-282 N287TMFFA (“TMFFA” disclosed as SEQ ID | | |
| | NO: 54) d288-291 d295 L298IM T300A I304E I309L P310L | | |
| | A314SHVRSV (“SHVRSV” disclosed as SEQ ID NO: 56) d315- | | |
| | 319 A328RELE (“RELE” disclosed as SEQ ID NO: 58) d329-331 | | |
| | T336K T339S N341H L351V K374T E375P I378P P386A Q389E | | |
| | R391K N394D V398N A402L D411TENAPR (“TENAPR” | | |
| | disclosed as SEQ ID NO: 63) d412-416 L419G D433K H436W | | |
| | E443RASFA (“RASFA” disclosed as SEQ ID NO: 66) d444-447 | | |
| | D455G Y459E R461G L471K K474EDGY (“EDGY” disclosed as | | |
| | SEQ ID NO: 68) d475-477 G506RHPD (“RHPD” disclosed as | | |
| | SEQ ID NO: 69) d507-509 T513A V521P D540T V542E T546K | | |
| | Q551R d562 I565F D566VE | | |
| 633 | D406V | 0.556 | − |
| 634 | T266Q | 0.057 | − |
| 635 | D373Q | −0.038 | − |
| 636 | S39A | 0.566 | − |
| 637 | V447A | 0.294 | − |
| 638 | N7T A8S Y19F G30D S39A V40L K44H Q47R L49Y A52K | −0.209 | − |
| | M54L K60R Y61F L64G R73G E78V S82A M83K L87VAV | | |
| | d88-89 V94TIEAA (“TIEAA” disclosed as SEQ ID NO: 19) d95- | | |
| | 98 I111VTPAN (“VTPAN” disclosed as SEQ ID NO: 20) d112- | | |
| | 115 S119T Q121D V129H T131G A138S A140K V144T T149R | | |
| | Y152C A159G E162SIDS (“SIDS” disclosed as SEQ ID NO: 95) | | |
| | d163-165 A170G H189G R213M S221A N227G E231Q S232D | | |
| | Q238DVS d239-240 F255ILLANVHY (“ILLANVHY” disclosed | | |
| | as SEQ ID NO: 36) d256-262 Y268IMNR (“IMNR” disclosed as | | |
| | SEQ ID NO: 43) d269-271 I275EEMLQAIQ (“EEML QAIQ” | | |
| | disclosed as SEQ ID NO: 46) d276-282 N287TFFFA (“TFFFA” | | |
| | disclosed as SEQ ID NO: 53) d288-291 d295 L298IM T300A | | |
| | I304E I309L P310L A314S M316V I319V A328RELE (“RELE” | | |
| | disclosed as SEQ ID NO: 58) d329-331 L335R T336K T339S | | |
| | N341H E348H L351V K374T E375P I378P P386A Q389E | | |
| | N394D V398N A402L D411TENAPR (“TENAPR” disclosed as | | |
| | SEQ ID NO: 63) d412-416 L419G D433K H436W E443RASFA | | |
| | (“RASFA” disclosed as SEQ ID NO: 66) d444-447 D455G | | |
| | Y459D R461G L471R K474EDGH (“EDGH” disclosed as SEQ | | |
| | ID NO: 67) d475-477 A508P Q509D T513A V521P D540T | | |
| | V542E T546K Q551R I562V I565V | | |
| 639 | A104T | 0.04 | − |
| 640 | N7T A8S Y19Q G30D S39A V40L K44H Q47R L49Y M54L | −0.089 | − |
| | K60R L64A Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140K V144T T149R | | |
| | Y152A A159G E162HFDSF (“HFDSF” disclosed as SEQ ID NO: | | |
| | 25) d163-166 A170G H189G R213M T220M S221A N227G | | |
| | E231QDLQ (“QDLQ” disclosed as SEQ ID NO: 33) d232-234 | | |
| | Q238D A240S F255I I257L P260V A262Y Y268IMNR (“IMNR” | | |
| | disclosed as SEQ ID NO: 43) d269-271 I275EEMLQAIQ | | |
| | (“EEMLQAIQ” disclosed as SEQ ID NO: 46) d276-282 | | |
| | N287TSMFA (“TSMFA” disclosed as SEQ ID NO: 70) d288-291 | | |
| | d295 L298IM T300A I304E I309L P310L A314S A318T I319V | | |
| | A328RELE (“RELE” disclosed as SEQ ID NO: 58) d329-331 | | |
| | T336K T339S N341T L351V K374P E375P I378P P386A Q389E | | |
| | R391K N394D V398N A402L D411TENAPR (“TENAPR” | | |
| | disclosed as SEQ ID NO: 63) d412-416 L419G D433K H436W | | |
| | E443RASFA (“RASFA” disclosed as SEQ ID NO: 66) d444-447 | | |
| | D455G Y459D R461G L471K K474EDGH (“EDGH” disclosed as | | |
| | SEQ ID NO: 67) d475-477 G506RHPD (“RHPD” disclosed as | | |
| | SEQ ID NO: 69) d507-509 T513A V521P D540T V542E T546K | | |
| | Q551R d562 I565F D566VE | | |
| 641 | N227T | 1.671 | + |
| 642 | L351F | 0.108 | − |
| 643 | Q121D | 1.584 | + |
| 644 | A264S | 0.51 | − |
| 645 | S232N | 4.536 | ++ |
| 646 | T215S | 0.865 | − |
| 647 | V277F | 0.416 | − |
| 648 | K489I | 0.328 | − |
| 649 | P260L | 0.471 | − |
| 650 | P528V | 0.893 | − |
| 651 | A234R | 0.495 | − |
| 652 | E348H | 0.287 | − |
| 653 | A229M | 2.152 | ++ |
| 654 | V472Y | 0.005 | − |
| 655 | T265K | 0.092 | − |
| 656 | E348Y | 0.48 | − |
| 657 | T362S | −0.072 | − |
| 658 | V472L | 0.234 | − |
| 659 | Y459E | −0.029 | − |
| 660 | N7T A8S Y19Q G30D S39A V40L K44H Q47R L49Y M54L | 0.01 | − |
| | K60R L64A Y69L R73G S82A M83K L87VAV d88-89 | | |
| | V94TIEAA (“TIEAA” disclosed as SEQ ID NO: 19) d95-98 | | |
| | I111V G114A S115N S119T Q121D V129H T131G A138S | | |
| | A140E V144T T149R Y152A A159G E162HFDAF (“HFDAF” | | |
| | disclosed as SEQ ID NO: 26) d163-166 A170G H189G R213M | | |
| | T220M S221A N227GKWSQDLQ (“GKWSQDLQ” disclosed as | | |
| | SEQ ID NO: 75) d228-234 Q238D A240S F255I I257L P260V | | |
| | Y268IMNR (“IMNR” disclosed as SEQ ID NO: 43) d269-271 | | |
| | I275EEML QAIQ (“EEML QAIQ” disclosed as SEQ ID NO: 46) | | |
| | d276-282 N287TSFFA (“TSFFA” disclosed as SEQ ID NO: 52) | | |
| | d288-291 d295 L298IM T300A I304E I309L P310L A314S | | |
| | A318T I319V A328RELE (“RELE” disclosed as SEQ ID NO: 58) | | |
| | d329-331 T336K T339S N341T L351V K374T E375P I378P | | |
| | Q389E R391K N394D V398N A402L D411TENAPR | | |
| | (“TENAPR” disclosed as SEQ ID NO: 63) d412-416 L419G | | |
| | D433K H436W E443RASFA (“RASFA” disclosed as SEQ ID | | |
| | NO: 66) d444-447 D455G Y459D R461G L471K K474EDGY | | |
| | (“EDGY” disclosed as SEQ ID NO: 68) d475-477 A508P Q509D | | |
| | T513A V521P D540T V542D T546A Q551R d562 I565F | | |
| | D566VE | | |
| 661 | P294A | 0.848 | − |
| 662 | A262L | 0.651 | − |
| 663 | Q238T | 3.582 | ++ |
| 664 | T235M | 0.344 | − |
| 665 | F255N | −0.023 | − |
| 666 | V15I | 1.077 | + |
| 667 | E231Q | 1.921 | + |
| 668 | D411T | 0.571 | − |
| 669 | H269M | 0.186 | − |
| 670 | F90V | 0.731 | − |
| 671 | V481T | 0.952 | − |
| 672 | N387S | 0.016 | − |
| 673 | G199P | 0.71 | − |
| 674 | A241I | −0.017 | − |
| 675 | F369C | 0.844 | − |
| 676 | K374P | 1.122 | + |
| 677 | Y555F | 1.446 | + |
| 678 | T331I | 1.596 | + |
| 679 | N7T A8S Y19Q D20W G30D S39A V40L A43G K44T Q47R | −0.073 | − |
| | M54L K60R L64A Y69L C72T R73G E78V S82N M83K L87V | | |
| | T89V V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129I T131G A138S A140E V144T T149R Y152I | | |
| | A159G E162HFDSF (“HFDSF” disclosed as SEQ ID NO: 25) | | |
| | d163-166 A170G H189G R213M T220M S221A N227GKWSQD | | |
| | (“GKWSQD” disclosed as SEQ ID NO: 28) d228-232 | | |
| | Q238DISAC (“DISAC” disclosed as SEQ ID NO: 34) d239-242 | | |
| | F255ILLANV (“ILLANV” disclosed as SEQ ID NO: 35) d256- | | |
| | 260 Y268IMNR (“IMNR” disclosed as SEQ ID NO: 43) d269-271 | | |
| | I275KEMLQAIQ (“KEMLQAIQ” disclosed as SEQ ID NO: 48) | | |
| | d276-282 N287TVFFA (“TVFFA” disclosed as SEQ ID NO: 51) | | |
| | d288-291 d295 L298IM T300A I304E I309L P310L A314S | | |
| | A318T I319V A328RELE (“RELE” disclosed as SEQ ID NO: 58) | | |
| | d329-331 L335R T336K T339S N341T L351V K374P E375P | | |
| | I378P P386A Q389E N394D V398N A402L D411TENAPR | | |
| | (“TENAPR” disclosed as SEQ ID NO: 63) d412-416 L419G | | |
| | D433K H436W E443RASFA (“RASFA” disclosed as SEQ ID | | |
| | NO: 66) d444-447 D455G Y459D R461G L471R K474EDGH | | |
| | (“EDGH” disclosed as SEQ ID NO: 67) d475-477 A508P Q509D | | |
| | T513A V521P D540T V542E T546K Q551R d562 I565F | | |
| | D566VE | | |
| 680 | L471I | 0.604 | − |
| 681 | L326I | 0.106 | − |
| 682 | K374W | 0.488 | − |
| 683 | Q355E | −0.062 | − |
| 684 | T546K | 1.054 | + |
| 685 | N7T A8S D20W G30D S39A V40L K44H Q47R L49Y M54L | −0.047 | − |
| | K60R L64A Y69L R73G S82G M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152I A159G E162HFDSF (“HFDSF” disclosed as SEQ ID NO: | | |
| | 25) d163-166 A170G H189G R213M S221A E224H N227G | | |
| | E231Q S232D Q238DVS d239-240 F255I I257L P260V A262Y | | |
| | Y268IMNR (“IMNR” disclosed as SEQ ID NO: 43) d269-271 | | |
| | L274PEEMLQAIQ (“PEEMLQAIQ” disclosed as SEQ ID NO: | | |
| | 45) d275-282 N287THFFA (“THFFA” disclosed as SEQ ID NO: | | |
| | 80) d288-291 d295 L298IM T300A I304E I309L P310L | | |
| | A314SHVRSV (“SHVRSV” disclosed as SEQ ID NO: 56) d315- | | |
| | 319 A328RELE (“RELE” disclosed as SEQ ID NO: 58) d329-331 | | |
| | L335R T336K T339S N341T L351V K374T E375P I378P P386A | | |
| | Q389E N394D V398N A402L D411TENAPD (“TENAPD” | | |
| | disclosed as SEQ ID NO: 61) d412-416 L419G D433K H436W | | |
| | E443RASFA (“RASFA” disclosed as SEQ ID NO: 66) d444-447 | | |
| | D455G Y459D R461G L471R K474EDGH (“EDGH” disclosed as | | |
| | SEQ ID NO: 67) d475-477 A508P Q509D T513A V521P D540T | | |
| | V542E T546K Q551R I562V I565V | | |
| 686 | A323G | 3.061 | ++ |
| 687 | N153S | 0.736 | − |
| 688 | K342F | 0.04 | − |
| 689 | T266C | 0.095 | − |
| 690 | D106K | −0.046 | − |
| 691 | R529A | −0.032 | − |
| 692 | N7T A8S Y19Q S39A V40L K44H Q47R L49Y M54L K60R | −0.073 | − |
| | L64A S67A Y69L R73G S82G M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140E V144T T149R | | |
| | Y152I A159G E162HFDAF (“HFDAF” disclosed as SEQ ID NO: | | |
| | 26) d163-166 A170G H189G R213M T220M S221A N227G | | |
| | E231Q S232D Q238DVSAC (“DVSAC” disclosed as SEQ ID | | |
| | NO: 81) d239-242 F255ILLANVHY (“ILLANVHY” disclosed as | | |
| | SEQ ID NO: 36) d256-262 Y268IMNR (“IMNR” disclosed as | | |
| | SEQ ID NO: 43) d269-271 L274PEEMLRAIQ (“PEEMLRAIQ” | | |
| | disclosed as SEQ ID NO: 44) d275-282 N287TVFFA (“TVFFA” | | |
| | disclosed as SEQ ID NO: 51) d288-291 d295 L298IM T300A | | |
| | I304E I309L P310L A314S A318S I319V A328RELE (“RELE” | | |
| | disclosed as SEQ ID NO: 58) d329-331 L335R T336K T339S | | |
| | N341T L351V K374T E375P I378P Q389E N394D V398N | | |
| | A402L D411TQNAPR (“TQNAPR” disclosed as SEQ ID NO: 62) | | |
| | d412-416 L419G D433K H436W E443RASFA (“RASFA” | | |
| | disclosed as SEQ ID NO: 66) d444-447 D455G Y459D R461G | | |
| | L471R K474EDGY (“EDGY” disclosed as SEQ ID NO: 68) d475- | | |
| | 477 A508P Q509D T513A V521P D540T V542E T546K Q551R | | |
| | I562V I565V D566E | | |
| 693 | G258H | 1.79 | + |
| 694 | Y478M | 0.135 | − |
| 695 | L335R | −0.117 | − |
| 696 | V338M | 0.659 | − |
| 697 | K554H | 1.974 | + |
| 698 | N7K A8S Y19Q G30D S39A V40L A43G K44S Q47R L49Y | −0.061 | − |
| | M54L K60R L64A Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129I T131G A138S A140E V144T T149R | | |
| | Y152A A159G E162HFDAF (“HFDAF” disclosed as SEQ ID | | |
| | NO: 26) d163-166 A170G H189G R213M T220M S221A | | |
| | N227GKWSQQ (“GKWSQQ” disclosed as SEQ ID NO: 90) | | |
| | d228-232 Q238DISAC (“DISAC” disclosed as SEQ ID NO: 34) | | |
| | d239-242 F255I I257L P260V A262Y Y268IMPR (“IMPR” | | |
| | disclosed as SEQ ID NO: 42) d269-271 I275EEMLQAIQ | | |
| | (“EEMLQAIQ” disclosed as SEQ ID NO: 46) d276-282 | | |
| | N287TVFFA (“TVFFA” disclosed as SEQ ID NO: 51) d288-291 | | |
| | d295 L298IM T300A I304E I309L P310L A314S A318T I319V | | |
| | A328RELE (“RELE” disclosed as SEQ ID NO: 58) d329-331 | | |
| | T336K T339S N341T L351V K374T E375P I378P P386A Q389E | | |
| | N394D V398N A402L D411TQNAPR (“TQNAPR” disclosed as | | |
| | SEQ ID NO: 62) d412-416 L419G D433K H436W E443RASFA | | |
| | (“RASFA” disclosed as SEQ ID NO: 66) d444-447 D455G | | |
| | Y459D R461G L471K K474EDGY (“EDGY” disclosed as SEQ | | |
| | ID NO: 68) d475-477 A508P Q509D T513A V521P D540T | | |
| | V542E T546K Q551K I562V I565V D566E | | |
| 699 | T360S | 0.331 | − |
| 700 | V40M | 0.058 | − |
| 701 | H189G | 0.912 | − |
| 702 | I10P | −0.02 | − |
| 703 | A470C | 0.469 | − |
| 704 | M503I | 0.502 | − |
| 705 | R379G | 0.418 | − |
| 706 | N7T A8S Y19Q D20W G30D S39A V40L K44H | −0.114 | − |
| | Q47RTYTQKEL (“RTYTQKEL” disclosed as SEQ ID NO: 15) | | |
| | d48-54 K60R L64A Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129I T131G A138S A140E V144T T149R | | |
| | Y152C A159G E162HFDAF (“HFDAF” disclosed as SEQ ID | | |
| | NO: 26) d163-166 A170G H189G R213M T220M S221A | | |
| | N227GKWSQD (“GKWSQD” disclosed as SEQ ID NO: 28) | | |
| | d228-232 Q238DVS d239-240 F255I I257L P260V A262Y | | |
| | Y268IMNR (“IMNR” disclosed as SEQ ID NO: 43) d269-271 | | |
| | L274PEEMLQAIQ (“PEEMLQAIQ” disclosed as SEQ ID NO: | | |
| | 45) d275-282 N287TFFFA (“TFFFA” disclosed as SEQ ID NO: | | |
| | 53) d288-291 d295 L298IM T300A I304E I309L P310L A314S | | |
| | I319V A328RELE (“RELE” disclosed as SEQ ID NO: 58) d329- | | |
| | 331 L335R T336K T339S N341T L351V K374T E375P I378P | | |
| | P386A Q389E R391K N394D V398N A402V D411TENAPD | | |
| | (“TENAPD” disclosed as SEQ ID NO: 61) d412-416 L419G | | |
| | D433K H436W E443RASFA (“RASFA” disclosed as SEQ ID | | |
| | NO: 66) d444-447 D455G Y459D R461G L471R K474EDGY | | |
| | (“EDGY” disclosed as SEQ ID NO: 68) d475-477 A508P Q509D | | |
| | T513A V521P D540T V542E T546K Q551R I562V I565V | | |
| 707 | R461G | 0.943 | − |
| 708 | H462G | −0.03 | − |
| 709 | P98L | −0.014 | − |
| 710 | N7T A8S Y19Q G30D S39A V40L K44H Q47R L49Y M54L | −0.143 | − |
| | K60R L64A Y69L C72T R73G E78V S82G M83R L87VA d89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152A A159G E162R L163I L166F A170G H189G R213M | | |
| | T220M S221A N227GKWSQ (“GKWSQ” disclosed as SEQ ID | | |
| | NO: 30) d228-231 Q238D A240S F255ILLLNV (“ILLLNV” | | |
| | disclosed as SEQ ID NO: 38) d256-260 Y268IMNR (“IMNR” | | |
| | disclosed as SEQ ID NO: 43) d269-271 I275EEMLQAIQ | | |
| | (“EEMLQAIQ” disclosed as SEQ ID NO: 46) d276-282 | | |
| | N287TMFFA (“TMFFA” disclosed as SEQ ID NO: 54) d288-291 | | |
| | d295 L298IM T300A I304E I309L P310L A314SHVRSV | | |
| | (“SHVRSV” disclosed as SEQ ID NO: 56) d315-319 A328RELE | | |
| | (“RELE” disclosed as SEQ ID NO: 58) d329-331 L335R T336K | | |
| | T339S N341T E348H L351V K374P E375P I378P P386A Q389E | | |
| | N394D V398N L410QTENNPE (“QTENNPE” disclosed as SEQ | | |
| | ID NO: 96) d411-416 L419G D433K H436W E443RASFA | | |
| | (“RASFA” disclosed as SEQ ID NO: 66) d444-447 D455G | | |
| | Y459D R461G L471R K474EDGH (“EDGH” disclosed as SEQ | | |
| | ID NO: 67) d475-477 A508P Q509D T513A V521P D540T | | |
| | V542E T546K Q551R d562 I565FV | | |
| 711 | N7T A8S Y19Q G30D S39A V40L A43G K44T Q47R L49Y | 0.02 | − |
| | M54L K60R L64A Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D F127MRHTG (“MRHTG” disclosed as SEQ ID | | |
| | NO: 21) d128-131 A138S A140E V144T T149R Y152A A159G | | |
| | E162HFDSF (“HFDSF” disclosed as SEQ ID NO: 25) d163-166 | | |
| | A170G H189G R213M T220M S221A N227G S232D | | |
| | Q238DISAC (“DISAC” disclosed as SEQ ID NO: 34) d239-242 | | |
| | F255I I257L P260V Y268IMNR (“IMNR” disclosed as SEQ ID | | |
| | NO: 43) d269-271 I275EEMLQAIQ (“EEML QAIQ” disclosed as | | |
| | SEQ ID NO: 46) d276-282 N287TVFFA (“TVFFA” disclosed as | | |
| | SEQ ID NO: 51) d288-291 d295 L298IM T300A I304E I309L | | |
| | P310L A314SHVRSV (“SHVRSV” disclosed as SEQ ID NO: 56) | | |
| | d315-319 A328RELE (“RELE” disclosed as SEQ ID NO: 58) | | |
| | d329-331 T336K T339S N341T L351V K374T E375P I378P | | |
| | P386A Q389E N394D V398N D411TENAPD (“TENAPD” | | |
| | disclosed as SEQ ID NO: 61) d412-416 L419G D433K H436W | | |
| | E443RASFA (“RASFA” disclosed as SEQ ID NO: 66) d444-447 | | |
| | D455G Y459D R461G L471K K474EDGY (“EDGY” disclosed as | | |
| | SEQ ID NO: 68) d475-477 G506RHPD (“RHPD” disclosed as | | |
| | SEQ ID NO: 69) d507-509 T513A V521P D540T V542D T546A | | |
| | Q551R d562 I565F D566VE | | |
| 712 | M83K | −0.017 | − |
| 713 | Y459D | −0.037 | − |
| 714 | T36H | 0.5 | − |
| 715 | K22S | 0.777 | − |
| 716 | F369L | 0.224 | − |
| 717 | Q389E | 0.136 | − |
| 718 | L384A | −0.083 | − |
| 719 | F272W | 0.556 | − |
| 720 | S217G | 0.64 | − |
| 721 | I414S | 0.358 | − |
| 722 | G271R | 1.112 | + |
| 723 | D370G | 0.343 | − |
| 724 | G199F | 0.122 | − |
| 725 | E164D | 0.619 | − |
| 726 | K139E | 1.024 | + |
| 727 | S490C | 1.397 | + |
| 728 | L249M | 1.251 | + |
| 729 | I239F | 0.109 | − |
| 730 | A109Q | −0.096 | − |
| 731 | I111M | 0.095 | − |
| 732 | N7T A8S Y19Q G30D S39A V40L K44T Q47R L49Y M54L | −0.068 | − |
| | K60R L64A Y69L C72T R73G S82A M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D F127MRHTG (“MRHTG” disclosed as SEQ ID | | |
| | NO: 21) d128-131 A138S A140E V144T T149R Y152C A159G | | |
| | E162HFDAF (“HFDAF” disclosed as SEQ ID NO: 26) d163-166 | | |
| | A170G H189G R213M T220M S221A N227G E231QDLK | | |
| | (“QDLK” disclosed as SEQ ID NO: 88) d232-234 Q238DISAC | | |
| | (“DISAC” disclosed as SEQ ID NO: 34) d239-242 | | |
| | F255ILLSNVHY (“ILLSNVHY” disclosed as SEQ ID NO: 41) | | |
| | d256-262 Y268IMPR (“IMPR” disclosed as SEQ ID NO: 42) | | |
| | d269-271 L274PEEMLQAIQ (“PEEML QAIQ” disclosed as SEQ | | |
| | ID NO: 45) d275-282 N287TMFHA (“TMFHA” disclosed as SEQ | | |
| | ID NO: 71) d288-291 d295 L298IM T300A I304E I309L P310L | | |
| | A314SHVRSV (“SHVRSV” disclosed as SEQ ID NO: 56) d315- | | |
| | 319 N329ELE d330-331 T336K T339S N341H L351V K374P | | |
| | E375P I378P P386A Q389E N394D V398N D411TENAPR | | |
| | (“TENAPR” disclosed as SEQ ID NO: 63) d412-416 L419G | | |
| | D433K H436W E443RASFA (“RASFA” disclosed as SEQ ID | | |
| | NO: 66) d444-447 D455G Y459D R461G L471R K474EDGH | | |
| | (“EDGH” disclosed as SEQ ID NO: 67) d475-477 A508P Q509D | | |
| | T513A V521P D540T V542E T546K Q551R d562 I565FV | | |
| 733 | F127L | 0.389 | − |
| 734 | Q491N | 0.872 | − |
| 735 | L471R | 1.246 | + |
| 736 | R376Q | 0.59 | − |
| 737 | V297L | 0.828 | − |
| 738 | W289L | 0.965 | − |
| 739 | F255Y | 0.179 | − |
| 740 | P260M | 0.703 | − |
| 741 | I296L | 1.133 | + |
| 742 | F91L | 0.119 | − |
| 743 | L351A | −0.042 | − |
| 744 | A262V | 0.319 | − |
| 745 | I243C | 2.345 | ++ |
| 746 | N7T A8S Y19Q G30D S39A V40L K44H Q47R L49Y M54L | −0.015 | − |
| | K60R L64A Y69L R73G E78V S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152T A159G E162RIESF (“RIESF” disclosed as SEQ ID NO: | | |
| | 24) d163-166 A170G H189G R213M T220M S221A N227G | | |
| | E231QNLQ (“QNLQ” disclosed as SEQ ID NO: 97) d232-234 | | |
| | Q238D F255ILLANVHY (“ILLANVHY” disclosed as SEQ ID | | |
| | NO: 36) d256-262 Y268IMNR (“IMNR” disclosed as SEQ ID | | |
| | NO: 43) d269-271 I275EEMLQAIQ (“EEML QAIQ” disclosed as | | |
| | SEQ ID NO: 46) d276-282 N287THMFV (“THMFV” disclosed as | | |
| | SEQ ID NO: 98) d288-291 d295 L298IM T300A I304E I309L | | |
| | P310L A314SHVRSV (“SHVRSV” disclosed as SEQ ID NO: 56) | | |
| | d315-319 A328RELE (“RELE” disclosed as SEQ ID NO: 58) | | |
| | d329-331 L335R T336K T339S N341T L351V K374P E375P | | |
| | I378P Q389E N394D V398N D411TENAPR (“TENAPR” | | |
| | disclosed as SEQ ID NO: 63) d412-416 L419G D433K H436W | | |
| | F443RASFA (“RASFA” disclosed as SEO ID NO: 66) d444-447 | | |
| | D455G Y459D R461G L471R K474EDGH (“EDGH” disclosed as | | |
| | SEQ ID NO: 67) d475-477 G506RHPD (“RHPD” disclosed as | | |
| | SEQ ID NO: 69) d507-509 T513A V521P D540T V542E T546K | | |
| | Q551R d562 I565FV | | |
| 747 | A262S | 0.696 | − |
| 748 | I239R | 0.037 | − |
| 749 | S490V | 0.793 | − |
| 750 | L298F | 0.067 | − |
| 751 | K383P | −0.009 | − |
| 752 | F250W | 0.431 | − |
| 753 | A318H | 1.778 | + |
| 754 | P260V | 0.17 | − |
| 755 | E372S | 1.236 | + |
| 756 | A264V | 0.554 | − |
| 757 | V354S | 0.126 | − |
| 758 | A229H | 0.237 | − |
| 759 | Q557K | −0.012 | − |
| 760 | L96E | 0.772 | − |
| 761 | R352D | 0.12 | − |
| 762 | I518V | 1.51 | + |
| 763 | V472M | −0.007 | − |
| 764 | V212C | 1.065 | + |
| 765 | Y539V | 0.35 | − |
| 766 | A211G | 0.331 | − |
| 767 | S288V | 1.102 | + |
| 768 | V286C | 0.124 | − |
| 769 | L105A | 0.113 | − |
| 770 | W289F | 0.075 | − |
| 771 | S230Q | 2.311 | ++ |
| 772 | S230A | 2.277 | ++ |
| 773 | R317T | −0.002 | − |
| 774 | Y152C | 0.413 | − |
| 775 | A234Q | 0.953 | − |
| 776 | R376H | 0.681 | − |
| 777 | S288T | 1.715 | + |
| 778 | V398N | 1.002 | + |
| 779 | A470G | 1.158 | + |
| 780 | K523M | 1.658 | + |
| 781 | N7T A8S Y19Q G30D S39A V40L K44H Q47R L49Y M54L | −0.192 | − |
| | K60R L64A Y69L R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152C A159G E162HFDSF (“HFDSF” disclosed as SEQ ID NO: | | |
| | 25) d163-166 A170G H189G R213M T220M S221A N227G | | |
| | E231Q L233Y Q238D A240S F255ILLLNVHY (“ILLLNVHY” | | |
| | disclosed as SEQ ID NO: 37) d256-262 Y268IMNR (“IMNR” | | |
| | disclosed as SEQ ID NO: 43) d269-271 I275EEMLQAIQ | | |
| | (“EEMLQAIQ” disclosed as SEQ ID NO: 46) d276-282 | | |
| | N287THMFA (“THMFA” disclosed as SEQ ID NO: 93) d288-291 | | |
| | d295 L298IM T300A I304E I309L P310L A314S I319V | | |
| | A328RELE (“RELE” disclosed as SEQ ID NO: 58) d329-331 | | |
| | L335R T336K T339S N341T L351V K374Q E375P I378P P386A | | |
| | Q389E N394D V398N A402L D411TENAPR (“TENAPR” | | |
| | disclosed as SEQ ID NO: 63) d412-416 L419G D433K H436W | | |
| | E443RASFA (“RASFA” disclosed as SEQ ID NO: 66) d444-447 | | |
| | D455G Y459D R461G L471R K474EDGH (“EDGH” disclosed as | | |
| | SEQ ID NO: 67) d475-477 A508P Q509D T513A V521P D540T | | |
| | V542E T546K Q551R d562 I565F D566VE | | |
| 782 | M479F | 0.832 | − |
| 783 | A318V | 4.372 | ++ |
| 784 | W102H | −0.12 | − |
| 785 | S232K | 1.323 | + |
| 786 | A234P | 0.534 | − |
| 787 | A234G | 0.805 | − |
| 788 | R465H | −0.03 | − |
| 789 | A242C | 0.585 | − |
| 790 | A240S | 1.518 | + |
| 791 | H261L | 0.168 | − |
| 792 | Q51H | −0.004 | − |
| 793 | H377R | 0.454 | − |
| 794 | V330I | 1.284 | + |
| 795 | M316I | 0.039 | − |
| 796 | A240L | 0.123 | − |
| 797 | K374E | 2.156 | ++ |
| 798 | E35G | 0.186 | − |
| 799 | V144T | 1.443 | + |
| 800 | K44D | 0.297 | − |
| 801 | S201Y | −0.088 | − |
| 802 | C222N | 0.599 | − |
| 803 | L320W | 4.521 | ++ |
| 804 | L87I | 1.036 | + |
| 805 | I257N | −0.125 | − |
| 806 | E443K | 1.55 | + |
| 807 | L198M | 0.133 | − |
| 808 | I275K | 0.947 | − |
| 809 | M83S | −0.009 | − |
| 810 | I378A | 1.431 | + |
| 811 | S253V | 0.466 | − |
| 812 | A328R | 2.098 | ++ |
| 813 | C72G | 0.18 | − |
| 814 | N7T A8S D20W G30D S39A V40L K44H Q47R L49Y M54L | −0.182 | − |
| | K60R L64A Y69L C72T R73G S82G M83K L87VAV d88-89 | | |
| | V94TIEAA (“TIEAA” disclosed as SEQ ID NO: 19) d95-98 | | |
| | I111V G114A S115N S119T Q121D V129H T131G A138S | | |
| | A140E V144T T149R Y152A A159G E162HFDSF (“HFDSF” | | |
| | disclosed as SEQ ID NO: 25) d163-166 A170G H189G R213M | | |
| | S221A E224H N227G E231Q S232Q Q238D A240S F255I I257L | | |
| | P260V A262Y Y268IMNR (“IMNR” disclosed as SEQ ID NO: | | |
| | 43) d269-271 I275K V277M A280AI d282 N287TVFFA | | |
| | (“TVFFA” disclosed as SEQ ID NO: 51) d288-291 d295 L298IM | | |
| | T300A I304E I309L P310L A314SHVRSV (“SHVRSV” | | |
| | disclosed as SEQ ID NO: 56) d315-319 A328RELE (“RELE” | | |
| | disclosed as SEQ ID NO: 58) d329-331 T336K T339S N341T | | |
| | E348Y L351V K374P E375P I378P P386A Q389E N394D | | |
| | V398N A402L D411TENAPD (“TENAPD” disclosed as SEQ ID | | |
| | NO: 61) d412-416 L419G D433K H436W E443RASFA | | |
| | (“RASFA” disclosed as SEQ ID NO: 66) d444-447 D455G | | |
| | Y459D R461G L471R K474EDGH (“EDGH” disclosed as SEQ | | |
| | ID NO: 67) d475-477 A508P Q509D T513A V521P D540T | | |
| | V542D T546A Q551K d562 I565FV | | |
| 815 | N7T A8S D20W G30D S39A V40L K44H Q47RTYTQKEL | 0.022 | − |
| | (“RTYTQKEL” disclosed as SEQ ID NO: 15) d48-54 K60R L64A | | |
| | Y69L C72T R73G S82N M83K L87VAV d88-89 V94TIEAA | | |
| | (“TIEAA” disclosed as SEQ ID NO: 19) d95-98 I111V G114A | | |
| | S115N S119T Q121D F127MRHTG (“MRHTG” disclosed as | | |
| | SEQ ID NO: 21) d128-131 A138S A140D V144T T149R Y152C | | |
| | A159G E162HFDAF (“HFDAF” disclosed as SEQ ID NO: 26) | | |
| | d163-166 A170G H189G R213M S221A E224H | | |
| | N227GKWSQDLK (“GKWSQDLK” disclosed as SEQ ID NO: | | |
| | 31) d228-234 Q238DISAC (“DISAC” disclosed as SEQ ID NO: | | |
| | 34) d239-242 F255ILLSNVHY (“ILLSNVHY” disclosed as SEQ | | |
| | ID NO: 41) d256-262 Y268IMNR (“IMNR” disclosed as SEQ ID | | |
| | NO: 43) d269-271 I275EEMLQAIQ (“EEMLQAIQ” disclosed as | | |
| | SEQ ID NO: 46) d276-282 N287TMFHA (“TMFHA” disclosed as | | |
| | SEQ ID NO: 71) d288-291 d295 L298IM T300A I304E I309L | | |
| | P310L A314SHVRSV (“SHVRSV” disclosed as SEQ ID NO: 56) | | |
| | d315-319 A328RELE (“RELE” disclosed as SEQ ID NO: 58) | | |
| | d329-331 L335R T336K T339S N341H L351V K374T E375P | | |
| | I378P Q389E R391K N394D V398N A402L D411TENAPR | | |
| | (“TENAPR” disclosed as SEQ ID NO: 63) d412-416 L419G | | |
| | D433K H436W E443RASFA (“RASFA” disclosed as SEQ ID | | |
| | NO: 66) d444-447 D455G Y459D R461G L471R K474EDGH | | |
| | (“EDGH” disclosed as SEQ ID NO: 67) d475-477 A508P Q509D | | |
| | T513A V521P D540T V542D T546A Q551R I562V I565V | | |
| 816 | I319V | 0.216 | − |
| 817 | N7T A8S Y19Q G30D S39A V40L K44H Q47R L49Y M54L | −0.064 | − |
| | K60R L64A Y69L R73G E78V S82N M83K L87VA d89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140E V144T T149R | | |
| | Y152I A159G E162R L163I L166F A170G H189G R213M | | |
| | T220M S221A N227G E231HDLK (“HDLK” disclosed as SEQ | | |
| | ID NO: 99) d232-234 Q238D A240S F255ILLSNV (“ILLSNV” | | |
| | disclosed as SEQ ID NO: 89) d256-260 Y268IMNR (“IMNR” | | |
| | disclosed as SEQ ID NO: 43) d269-271 I275EEMLQAIQ | | |
| | (“EEMLQAIQ” disclosed as SEQ ID NO: 46) d276-282 | | |
| | N287TMFHA (“TMFHA” disclosed as SEQ ID NO: 71) d288-291 | | |
| | d295 L298IM T300A I304E I309L P310L A314SHVRSV | | |
| | (“SHVRSV” disclosed as SEQ ID NO: 56) d315-319 A328RELE | | |
| | (“RELE” disclosed as SEQ ID NO: 58) d329-331 L335R T336K | | |
| | T339S N341T L351V K374T E375P I378P P386A Q389E N394D | | |
| | V398N A402L D411TENAPR (“TENAPR” disclosed as SEQ ID | | |
| | NO: 63) d412-416 L419G D433K H436W E443RASFA | | |
| | (“RASFA” disclosed as SEQ ID NO: 66) d444-447 D455G | | |
| | Y459D R461G L471K K474EDGY (“EDGY” disclosed as SEQ | | |
| | ID NO: 68) d475-477 A508P Q509D T513A V521P D540T | | |
| | V542D T546A Q551R I562V I565V | | |
| 818 | A52K | 1.213 | + |
| 819 | D16N | 0.907 | − |
| 820 | G271Q | 1.531 | + |
| 821 | T220V | −0.017 | − |
| 822 | L410Q | 0.253 | − |
| 823 | N12H | 0.438 | − |
| 824 | S201L | 0.022 | − |
| 825 | S288C | 0.776 | − |
| 826 | H261S | 0.013 | − |
| 827 | L252Q | 0.041 | − |
| 828 | N442T | 0.945 | − |
| 829 | G258T | 1.285 | + |
| 830 | I10H | 0.581 | − |
| 831 | F369V | 0.571 | − |
| 832 | K374Q | 1.007 | + |
| 833 | P294T | 0.117 | − |
| 834 | A318W | 1.829 | + |
| 835 | S223L | 4.185 | ++ |
| 836 | S363G | 1.593 | + |
| 837 | L326C | −0.059 | − |
| 838 | G327S | 0.592 | − |
| 839 | Q494N | 1.757 | + |
| 840 | S321C | 4.28 | ++ |
| 841 | P310D | 0.375 | − |
| 842 | K254M | 1.399 | + |
| 843 | F369W | 0.419 | − |
| 844 | W102F | −0.137 | − |
| 845 | D540T | −0.058 | − |
| 846 | D373E | 0.083 | − |
| 847 | A138S | 1.414 | + |
| 848 | N7T A8S Y19Q G30D S39A V40L K44H Q47R L49Y M54L | −0.104 | − |
| | K60R L64A Y69L C72T R73G S82G M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D F127MRHTG (“MRHTG” disclosed as SEQ ID | | |
| | NO: 21) d128-131 A138S A140E V144T T149R Y152I A159G | | |
| | E162HFDAF (“HFDAF” disclosed as SEQ ID NO: 26) d163-166 | | |
| | A170G M172L H189G R213M T220M S221A N227GKWSQD | | |
| | (“GKWSQD” disclosed as SEQ ID NO: 28) d228-232 Q238D | | |
| | A240S F255ILLANV (“ILLANV” disclosed as SEQ ID NO: 35) | | |
| | d256-260 Y268IMNR (“IMNR” disclosed as SEQ ID NO: 43) | | |
| | d269-271 I275KEMLQAIQ (“KEML QAIQ” disclosed as SEQ ID | | |
| | NO: 48) d276-282 N287TVFFA (“TVFFA” disclosed as SEQ ID | | |
| | NO: 51) d288-291 d295 L298IM T300A I304E I309L P310L | | |
| | A314SHVRTV (“SHVRTV” disclosed as SEQ ID NO: 57) d315- | | |
| | 319 A328RELE (“RELE” disclosed as SEQ ID NO: 58) d329-331 | | |
| | T336K T339S N341T L351V K374P E375P I378P P386A Q389E | | |
| | N394D V398N A402L D411TENAPR (“TENAPR” disclosed as | | |
| | SEQ ID NO: 63) d412-416 L419G D433K H436W E443RASFA | | |
| | (“RASFA” disclosed as SEQ ID NO: 66) d444-447 D455G | | |
| | Y459D R461G L471K K474EDGH (“EDGH” disclosed as SEQ | | |
| | ID NO: 67) d475-477 A508P Q509D T513A V521P D540T | | |
| | V542E T546K Q551R d562 I565FV | | |
| 849 | M54L | 1.257 | + |
| 850 | G199C | 0.9 | − |
| 851 | G199A | 2.481 | ++ |
| 852 | A262N | 1.976 | + |
| 853 | Q281L | 0.261 | − |
| 854 | V15P | 0.547 | − |
| 855 | I319L | 0.13 | − |
| 856 | K523Y | 1.478 | + |
| 857 | D370N | 0.327 | − |
| 858 | A318I | 3.021 | ++ |
| 859 | Q416E | 1.956 | + |
| 860 | R376D | 0.013 | − |
| 861 | L291G | −0.143 | − |
| 862 | K523D | 3.213 | ++ |
| 863 | A43G | 0.601 | − |
| 864 | E162R | 0.075 | − |
| 865 | E372R | 0.843 | − |
| 866 | N525G | 2.763 | ++ |
| 867 | W102L | −0.082 | − |
| 868 | Q238D | 1.144 | + |
| 869 | S202G | −0.002 | − |
| 870 | N7D | 0.588 | − |
| 871 | L233Y | 3.222 | ++ |
| 872 | L299F | 0.135 | − |
| 873 | A402I | 0.124 | − |
| 874 | F369S | 1.271 | + |
| 875 | Q281I | 0.456 | − |
| 876 | N259A | 1.175 | + |
| 877 | S223H | 3.028 | ++ |
| 878 | R477Y | 0.3 | − |
| 879 | N7T A8S Y19Q G30D T36H S39A V40L A43G K44T Q47RTYT | −0.052 | − |
| | (“RTYT” disclosed as SEQ ID NO: 17) d48-50 M54L K60R | | |
| | L64A Y69L R73G S82N M83K L87VAV d88-89 V94TIEAA | | |
| | (“TIEAA” disclosed as SEQ ID NO: 19) d95-98 I111VTPAN | | |
| | (“VTPAN” disclosed as SEQ ID NO: 20) d112-115 S119T Q121R | | |
| | V129H T131G A138S A140E V144T T149R Y152A A159G | | |
| | E162SIDSF (“SIDSF” disclosed as SEQ ID NO: 100) d163-166 | | |
| | A170G H189G R213M T220M S221A N227GKWSQD | | |
| | (“GKWSQD” disclosed as SEQ ID NO: 28) d228-232 | | |
| | Q238DISAC (“DISAC” disclosed as SEQ ID NO: 34) d239-242 | | |
| | F255I I257L P260V A262Y Y268IMNR (“IMNR” disclosed as | | |
| | SEQ ID NO: 43) d269-271 I275EEMLRAIQ (“EEMLRAIQ” | | |
| | disclosed as SEQ ID NO: 47) d276-282 N287TVFFA (“TVFFA” | | |
| | disclosed as SEQ ID NO: 51) d288-291 d295 L298IM T300A | | |
| | I304E I309L P310L A314SHVRTV (“SHVRTV” disclosed as | | |
| | SEQ ID NO: 57) d315-319 A328RELE (“RELE” disclosed as | | |
| | SEQ ID NO: 58) d329-331 T336K T339S N341H L351V K374T | | |
| | E375P I378P Q389E N394D V398N A402L D411TENAPR | | |
| | (“TENAPR” disclosed as SEQ ID NO: 63) d412-416 L419G | | |
| | D433K H436W E443RASFA (“RASFA” disclosed as SEQ ID | | |
| | NO: 66) d444-447 D455G Y459D R461G L471K K474EDGY | | |
| | (“EDGY” disclosed as SEQ ID NO: 68) d475-477 A508P Q509D | | |
| | T513A V521P D540T V542E T546K Q551R d562 I565F | | |
| | D566VE | | |
| 880 | P386A | 0.135 | − |
| 881 | W289I | −0.06 | − |
| 882 | Q551K | 0.304 | − |
| 883 | V100L | 1.12 | + |
| 884 | A318L | 3.019 | ++ |
| 885 | Y197C | 0.042 | − |
| 886 | A366T | 0.278 | − |
| 887 | E224N | 0.001 | − |
| 888 | A140D | 1.506 | + |
| 889 | N287T | 0.265 | − |
| 890 | F369T | 1.92 | + |
| 891 | T131D | 0.887 | − |
| 892 | L298R | 0.145 | − |
| 893 | V297T | 0.855 | − |
| 894 | V40L | 0.056 | − |
| 895 | Q51L | 0.179 | − |
| 896 | S66R | 1.288 | + |
| 897 | I13M | 0.083 | − |
| 898 | F91A | −0.125 | − |
| 899 | C381S | 0.459 | − |
| 900 | L233P | 0.557 | − |
| 901 | E372N | 2.105 | ++ |
| 902 | G258A | 1.898 | + |
| 903 | L256F | 0.005 | − |
| 904 | Q47R | 0.897 | − |
| 905 | A583E | 2.449 | ++ |
| 906 | D370Q | 0.096 | − |
| 907 | A240C | 1.159 | + |
| 908 | V15D | 0.09 | − |
| 909 | I319M | 0.117 | − |
| 910 | I111L | 0.302 | − |
| 911 | S247C | 0.834 | − |
| 912 | K374H | 1.148 | + |
| 913 | E486D | 1.205 | + |
| 914 | L334M | 0.283 | − |
| 915 | I195M | −0.12 | − |
| 916 | N7T A8S Y19Q S39A V40L A43G K44S Q47RTYTQKEL | −0.11 | − |
| | (“RTYTQKEL” disclosed as SEQ ID NO: 15) d48-54 K60R L64A | | |
| | Y69L C72T R73G S82N M83K L87VAV d88-89 V94TIEAA | | |
| | (“TIEAA” disclosed as SEQ ID NO: 19) d95-98 I111VTPAN | | |
| | (“VTPAN” disclosed as SEQ ID NO: 20) d112-115 S119T Q121D | | |
| | V129H T131G A138S A140E V144T T149R Y152A N153S | | |
| | A159G E162HFDSF (“HFDSF” disclosed as SEQ ID NO: 25) | | |
| | d163-166 A170G H189G R213M T220M S221A N227G E231Q | | |
| | S232D Q238DISAC (“DISAC” disclosed as SEQ ID NO: 34) | | |
| | d239-242 F255I I257L P260V Y268IMPR (“IMPR” disclosed as | | |
| | SEQ ID NO: 42) d269-271 I275Q V277M A280AI d282 | | |
| | N287TSFFA (“TSFFA” disclosed as SEQ ID NO: 52) d288-291 | | |
| | d295 L298IM T300A I304E I309L P310L A314SHVRWV | | |
| | (“SHVRWV” disclosed as SEQ ID NO: 101) d315-319 | | |
| | A328RELE (“RELE” disclosed as SEQ ID NO: 58) d329-331 | | |
| | T336K T339S N341T E348H L351V K374T E375P I378P P386A | | |
| | Q389E R391K N394D V398N A402L D411TENAPR | | |
| | (“TENAPR” disclosed as SEQ ID NO: 63) d412-416 L419G | | |
| | D433K H436W E443RASFA (“RASFA” disclosed as SEQ ID | | |
| | NO: 66) d444-447 D455G Y459D R461G L471R K474EDGY | | |
| | (“EDGY” disclosed as SEQ ID NO: 68) d475-477 G506RHPD | | |
| | (“RHPD” disclosed as SEQ ID NO: 69) d507-509 T513A V521P | | |
| | D540T V542E T546K Q551R d562 I565F D566VE | | |
| 917 | E224M | 0.93 | − |
| 918 | T235Q | 0.779 | − |
| 919 | C445S | 0.848 | − |
| 920 | N7S | 1.655 | + |
| 921 | T290L | 0.412 | − |
| 922 | L384M | −0.002 | − |
| 923 | K493Y | 1.712 | + |
| 924 | G506R | 0.776 | − |
| 925 | I111C | 0.151 | − |
| 926 | I480L | 0.872 | − |
| 927 | E231P | 2.566 | ++ |
| 928 | T367H | 0.023 | − |
| 929 | F255C | 0.358 | − |
| 930 | L163F | 0.061 | − |
| 931 | S232Q | 1.852 | + |
| 932 | S221A | 1.424 | + |
| 933 | V354I | 0.735 | − |
| 934 | Y137T | 0.058 | − |
| 935 | N387H | 0.034 | − |
| 936 | I225Q | 0.144 | − |
| 937 | H377N | 0.918 | − |
| 938 | I414N | 0.445 | − |
| 939 | Y152I | 0.247 | − |
| 940 | L198T | −0.041 | − |
| 941 | L334I | 0.204 | − |
| 942 | P364S | 4.696 | ++ |
| 943 | Q551R | 0.118 | − |
| 944 | N7T A8S Y19F G30D S39A V40L K44H Q47R L49Y M54L | −0.104 | − |
| | K60R L64A Y69L C72T R73G S82G M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D F127MRHTG (“MRHTG” disclosed as SEQ ID | | |
| | NO: 21) d128-131 A138S A140E V144T T149R Y152C A159G | | |
| | E162HFDAF (“HFDAF” disclosed as SEQ ID NO: 26) d163-166 | | |
| | A170G H189G R213M S221A N227G E231QDLT (“QDLT” | | |
| | disclosed as SEQ ID NO: 102) d232-234 Q238D A240S | | |
| | F255ILLHNV (“ILLHNV” disclosed as SEQ ID NO: 103) d256- | | |
| | 260 Y268IMNR (“IMNR” disclosed as SEQ ID NO: 43) d269-271 | | |
| | I275EEMLRAIQ (“EEMLRAIQ” disclosed as SEQ ID NO: 47) | | |
| | d276-282 N287TVFFA (“TVFFA” disclosed as SEQ ID NO: 51) | | |
| | d288-291 d295 L298IM T300A I304E I309L P310L | | |
| | A314SHVRTV (“SHVRTV” disclosed as SEQ ID NO: 57) d315- | | |
| | 319 A328RELE (“RELE” disclosed as SEQ ID NO: 58) d329-331 | | |
| | T336K T339S N341T L351V K374T E375P I378P P386A Q389E | | |
| | N394D V398N D411TENAPR (“TENAPR” disclosed as SEQ ID | | |
| | NO: 63) d412-416 L419G D433K H436W E443RASFA | | |
| | (“RASFA” disclosed as SEQ ID NO: 66) d444-447 D455G | | |
| | Y459D R461G L471R K474EDGH (“EDGH” disclosed as SEQ | | |
| | ID NO: 67) d475-477 A508P Q509D T513A V521P D540T | | |
| | V542E T546K Q551R d562 I565F D566VE | | |
| 945 | A496Y | 1.161 | + |
| 946 | L163I | 0.592 | − |
| 947 | I304E | 1.17 | + |
| 948 | N227A | 1.728 | + |
| 949 | N7T A8S Y19F S39A V40L A43G K44S Q47R L49Y M54L | −0.099 | − |
| | K60R L64A Y69L R73G S82N M83K L87V T89V V94TIEAA | | |
| | (“TIEAA” disclosed as SEQ ID NO: 19) d95-98 I111V G114A | | |
| | S115N S119T Q121R F127MRITG (“MRITG” disclosed as SEQ | | |
| | ID NO: 22) d128-131 A138S A140E V144T T149R Y152A | | |
| | A159G E162HFDAF (“HFDAF” disclosed as SEQ ID NO: 26) | | |
| | d163-166 A170G H189G R213M S221A N227G E231QDY d232- | | |
| | 233 Q238DVSAC (“DVSAC” disclosed as SEQ ID NO: 81) d239- | | |
| | 242 F255ILLANVHY (“ILLANVHY” disclosed as SEQ ID NO: | | |
| | 36) d256-262 Y268IMPR (“IMPR” disclosed as SEQ ID NO: 42) | | |
| | d269-271 I275K V277M A280AI d282 N287TMFFA (“TMFFA” | | |
| | disclosed as SEQ ID NO: 54) d288-291 d295 L298IM T300A | | |
| | I309L P310L A314SHVRSV (“SHVRSV” disclosed as SEQ ID | | |
| | NO: 56) d315-319 A328RELE (“RELE” disclosed as SEQ ID NO: | | |
| | 58) d329-331 L335RKRM (“RKRM” disclosed as SEQ ID NO: | | |
| | 104) d336-338 E348H L351V K374T I378P P386A Q389E | | |
| | R391K N394D V398N A402L D411TENAPD (“TENAPD” | | |
| | disclosed as SEQ ID NO: 61) d412-416 L419G D433K H436W | | |
| | E443RASFA (“RASFA” disclosed as SEQ ID NO: 66) d444-447 | | |
| | D455G Y459D R461G L471K K474EDGY (“EDGY” disclosed as | | |
| | SEQ ID NO: 68) d475-477 G506RHPD (“RHPD” disclosed as | | |
| | SEQ ID NO: 69) d507-509 T513A V521P D540T V542D T546A | | |
| | Q551R d562 I565F D566VE | | |
| 950 | F369M | 2.378 | ++ |
| 951 | A234H | 3.073 | ++ |
| 952 | N525E | 2.289 | ++ |
| 953 | T336K | 1.648 | + |
| 954 | T513V | 2.912 | ++ |
| 955 | M479W | 0.545 | − |
| 956 | K254F | 0.985 | − |
| 957 | L351W | −0.025 | − |
| 958 | C445A | 1.34 | + |
| 959 | S205T | 0.682 | − |
| 960 | T235D | 0.931 | − |
| 961 | S82N | 0.792 | − |
| 962 | V390A | 1.434 | + |
| 963 | A8I | 0.005 | − |
| 964 | R391K | 0.101 | − |
| 965 | E231H | 1.243 | + |
| 966 | N7T A8S D20W G30D S39A V40L K44H Q47R L49Y M54L | −0.058 | − |
| | K60R L64A Y69L C72T R73G S82N M83K L87V T89V | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140E V144T T149R | | |
| | Y152I A159G E162HFDSF (“HFDSF” disclosed as SEQ ID NO: | | |
| | 25) d163-166 A170G H189G R213M S221A E224H N227G | | |
| | E231QL233Y Q238DISAC (“DISAC” disclosed as SEQ ID NO: | | |
| | 34) d239-242 F255ILLLNVHY (“ILLLNVHY” disclosed as SEQ | | |
| | ID NO: 37) d256-262 Y268IMNR (“IMNR” disclosed as SEQ ID | | |
| | NO: 43) d269-271 L274PEEMLQAIQ (“PEEMLQAIQ” disclosed | | |
| | as SEQ ID NO: 45) d275-282 N287TVFFA (“TVFFA” disclosed | | |
| | as SEQ ID NO: 51) d288-291 d295 L298IM T300A I304E I309L | | |
| | P310L A314SHVRTV (“SHVRTV” disclosed as SEQ ID NO: 57) | | |
| | d315-319 A328RELE (“RELE” disclosed as SEQ ID NO: 58) | | |
| | d329-331 L335R T336K T339S N341H L351V K374T E375P | | |
| | I378P P386A Q389E R391K N394D V398N A402L | | |
| | D411TENAPR (“TENAPR” disclosed as SEQ ID NO: 63) d412- | | |
| | 416 L419G D433K H436W E443RASFA (“RASFA” disclosed as | | |
| | SEQ ID NO: 66) d444-447 D455G Y459D R461G L471K | | |
| | K474EDGY (“EDGY” disclosed as SEQ ID NO: 68) d475-477 | | |
| | G506RHPD (“RHPD” disclosed as SEQ ID NO: 69) d507-509 | | |
| | T513A V521P D540T V542E T546K Q551R d562 I565F | | |
| | D566VE | | |
| 967 | A234E | 0.549 | − |
| 968 | T513C | 3.65 | ++ |
| 969 | T235G | 0.809 | − |
| 970 | N12P | 1.611 | + |
| 971 | A240V | 0.248 | − |
| 972 | P553S | 0.053 | − |
| 973 | K254S | 0.225 | − |
| 974 | Q51Y | 0.08 | − |
| 975 | N7T A8S Y19Q G30D S39A V40L A43G K44S Q47R L49Y | −0.002 | − |
| | M54L K60R L64A Y69L C72T R73G S82N M83K L87VAV | | |
| | d88-89 V94TIEAA (“TIEAA” disclosed as SEQ ID NO: 19) d95- | | |
| | 98 I111VTPAN (“VTPAN” disclosed as SEQ ID NO: 20) d112- | | |
| | 115 S119T Q121D F127MRHTG (“MRHTG” disclosed as SEQ | | |
| | ID NO: 21) d128-131 A138S A140E V144T T149R Y152C | | |
| | A159G E162HFDAF (“HFDAF” disclosed as SEQ ID NO: 26) | | |
| | d163-166 A170G H189G R213M T220M S221A | | |
| | N227GKWSQDLK (“GKWSQDLK” disclosed as SEQ ID NO: | | |
| | 31) d228-234 Q238DISAC (“DISAC” disclosed as SEQ ID NO: | | |
| | 34) d239-242 F255ILLSNVHY (“ILLSNVHY” disclosed as SEQ | | |
| | ID NO: 41) d256-262 Y268IMPR (“IMPR” disclosed as SEQ ID | | |
| | NO: 42) d269-271 I275KEMLQAIQ (“KEMLQAIQ” disclosed as | | |
| | SEQ ID NO: 48) d276-282 N287TMFHA (“TMFHA” disclosed as | | |
| | SEQ ID NO: 71) d288-291 d295 L298IM T300A I304E E306D | | |
| | I309L P310L A314SHVRSV (“SHVRSV” disclosed as SEQ ID | | |
| | NO: 56) d315-319 A328RELE (“RELE” disclosed as SEQ ID NO: | | |
| | 58) d329-331 L335R T336K T339S N341T L351V K374T E375P | | |
| | I378P P386A Q389E N394D V398N A402L D411TQNAPD | | |
| | (“TQNAPD” disclosed as SEQ ID NO: 92) d412-416 L419G | | |
| | D433K H436W E443RASFA (“RASFA” disclosed as SEQ ID | | |
| | NO: 66) d444-447 D455G Y459D R461G L471K K474EDGY | | |
| | (“EDGY” disclosed as SEQ ID NO: 68) d475-477 A508P Q509D | | |
| | T513A V521P D540T V542D T546A Q551R d562 I565F | | |
| | D566VE | | |
| 976 | S253M | 1.746 | + |
| 977 | C381I | 0.356 | − |
| 978 | N7T A8S Y19Q G30D S39A V40L K44H Q47R L49Y M54L | −0.146 | − |
| | K60R L64A Y69L C72T R73G S82N M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121R V129H T131G A138S A140E V144T T149R | | |
| | Y152I A159G E162HFDSF (“HFDSF” disclosed as SEQ ID NO: | | |
| | 25) d163-166 A170G H189G R213M T220M S221A | | |
| | N227GKWSQ (“GKWSQ” disclosed as SEQ ID NO: 30) d228- | | |
| | 231 Q238D A240S F255I I257L P260V A262Y Y268IMNR | | |
| | (“IMNR” disclosed as SEQ ID NO: 43) d269-271 | | |
| | I275EEMLQAIQ (“EEMLQAIQ” disclosed as SEQ ID NO: 46) | | |
| | d276-282 N287TVFFA (“TVFFA” disclosed as SEQ ID NO: 51) | | |
| | d288-291 d295 L298IM T300A I304E I309L P310L | | |
| | A314SHVRSV (“SHVRSV” disclosed as SEQ ID NO: 56) d315- | | |
| | 319 A328RELE (“RELE” disclosed as SEQ ID NO: 58) d329-331 | | |
| | L335Q T336K L351V K374T I378P Q389E R391K N394D | | |
| | V398N A402L D411TENAPR (“TENAPR” disclosed as SEQ ID | | |
| | NO: 63) d412-416 L419G D433K H436W E443RASFA | | |
| | (“RASFA” disclosed as SEQ ID NO: 66) d444-447 D455G | | |
| | Y459D R461G L471K K474EDGY (“EDGY” disclosed as SEQ | | |
| | ID NO: 68) d475-477 A508P Q509D T513A V521P D540T | | |
| | V542E T546K Q551R d562 I565FV | | |
| 979 | A328S | 0.997 | − |
| 980 | I243M | 2.058 | ++ |
| 981 | I244V | 0.642 | − |
| 982 | L105I | 0.504 | − |
| 983 | K254G | −0.044 | − |
| 984 | S232D | 0.367 | − |
| 985 | Y69L | 0.05 | − |
| 986 | L291V | 0.67 | − |
| 987 | G31A | 0.018 | − |
| 988 | S191A | 1.377 | + |
| 989 | K493L | 1.43 | + |
| 990 | N7K A8S D20W G30D S39A V40L K44H Q47R L49Y M54L | 0.005 | − |
| | K60R L64A Y69L R73G S82G M83K L87VAV d88-89 | | |
| | V94TIEAA (“TIEAA” disclosed as SEQ ID NO: 19) d95-98 | | |
| | I111VTPAN (“VTPAN” disclosed as SEQ ID NO: 20) d112-115 | | |
| | S119T Q121D V129H T131G A138S A140E V144T T149R | | |
| | Y152I A159G E162HFDSF (“HFDSF” disclosed as SEQ ID NO: | | |
| | 25) d163-166 A170G H189G R213M S221A E224H N227G | | |
| | E231Q Q238D A240S F255ILLHNVHM (“ILLHNVHM” | | |
| | disclosed as SEQ ID NO: 83) d256-262 Y268IMNR (“IMNR” | | |
| | disclosed as SEQ ID NO: 43) d269-271 L274PEEML QAIQ | | |
| | (“PEEMLQAIQ” disclosed as SEQ ID NO: 45) d275-282 | | |
| | N287TVFFA (“TVFFA” disclosed as SEQ ID NO: 51) d288-291 | | |
| | d295 L298IM T300A I304E I309L P310L A314S A318T I319V | | |
| | A328RELE (“RELE” disclosed as SEQ ID NO: 58) d329-331 | | |
| | L335R T336K T339S N341T L351V K374P E375P I378P P386A | | |
| | Q389E N394D V398N A402L D411TENAPR (“TENAPR” | | |
| | disclosed as SEQ ID NO: 63) d412-416 L419G D433K H436W | | |
| | E443RASFA (“RASFA” disclosed as SEQ ID NO: 66) d444-447 | | |
| | D455G Y459D R461G L471R K474EDGH (“EDGH” disclosed as | | |
| | SEQ ID NO: 67) d475-477 G506RHPD (“RHPD” disclosed as | | |
| | SEQ ID NO: 69) d507-509 T513A V521P D540T V542D T546A | | |
| | Q551R d562 I565F D566VE | | |

*wherein a space indicates a comma, a lower case “d” proceeding the position indicates deletion, a hyphen
between two positions indicates alterations of the two positions and the including the range between the two
positions, identification of multiple residues means insertion of all identified residues.
Key

| | |
|---|---|
| − | lower activity than control <1 |
| + | similar activity to control ≥1 |
| ++ | higher activity than control >2 |

TABLE 9 shows that ACL variants of homolog 2 nos. 670, 742, 818, 471, 734, 40, 602, 616, 306, 393, 535, 420, 792, 693, 889, 797, 157, 244, 864, 328, 412, 842, 937, 865, 31, 612, 908, 695, 34, 668, 163, 663, 289, 823, 480, 3, 887, 102, 923, 373, 703, 713, 533, 481, 903, 539, 180, 364, 475, 705, 399, 580, 637, 477, 544, 30, 396, 461, 329, 752, 124, 150, 437, 803, 377, 771, 788, 26, 43, 698, 517, 315, 178, 427, 888, 483, 723, 737, 441, 448, 763, 559, 33, 308, 949, 554, 202, 478, 486, 442, 808, 946, 443, 596, 4, 782, 800, 529, 952, 789, 349, 709, 355, 817, 859, 379, 840, 959, 416, 435, 558, 623, 282, 419, 850, 541, 697, 358, 845, 757, 201, 799, 426, 120, 128, 646, 241, 714, 661, 927, 44, 780, 567, 929, 184, 323, 962, 404, 555, 754, 221, 750, 408, 45, 497, 774, 877, 776, 410, 174, 581, 521, 287, 796, 814, 758, 896, 760, 428, 245, 469, 424, 628, 920, 715, 277, 575, 186, 173, 761, 376, 524, 634, 356, 271, 819, 454, 470, 468, 196, 793, 943, 220, 655, 683, 868, 860, 68, 162, 855, 671, 597, 126, 336, 211, 566, 215, 658, 945, 638, 439, 807, 606, 59, 77, 712, 674, 872, 179, 357, 232, 479, 302, 721, 440, 879, 195, 786, 610, 6, 205, 956, 777, 688, 642, 914, 167, 513, 765, 609, 153, 14, 831, 158, 841, 394, 504, 577, 70, 557, 570, 17, 595, 746, 296, 310, 755, 247, 883, 100, 154, 209, 294, 64, 2, 276, 301, 676, 161, 97, 172, 885, 53, 591, 869, 725, 332, 592, 706, 119, 375, 84, 177, 337, 906, 334, 297, 83, 313, 873, 917, 108, 152, 307, 438, 208, 125, 260, 65, 185, 743, 718, 13, 619, 320, 362, 9, 339, 625, 735, 627, 954, 367, 311, 632, 656, 622, 390, 88, 238, 540, 564, 744, 36, 899, 729, 351, 457, 52, 547, 366, 265, 933, 114, 387, 275, 514, 431, 223, 812, 846, 653, 227, 340, 137, 447, 250, 707, 316, 924, 490, 25, 644, 109, 572, 142, 939, 47, 281, 827, 696, 904, 113, 702, 182, 226, 360, 498, 659, 409, 505, 159, 599, 123, 175, 305, 312, 164, 368, 928, 654, 436, 304, 263, 615, 825, 542, 690, 886, 861, 563, 93, 213, 371, 415, 881, 28, 665, 279, 813, 783, 801, 645, 488, 598, 926, 523, 197, 730, 719, 67, 749, 915, 958, 500, 75, 867, 252, and 745 have the highest activity level relative to homolog 2 and the highest activity levels on HHA.

TABLE 9
Exemplary Engineered ACLs Using Homolog 2 (SEQ
ID NO: 2) as the Template with Positional Alteration(s)
and Corresponding Activity on HHA

| | | Activity | |
|---|---|---|---|
| Variant | Mutations to | relative to | Active |
| # | homolog 2 | homolog 2 | HHA |
| | Homolog 2 | 1 | + |
| 1 | M99L | 1.337 | + |
| 2 | E142N | 3.636 | ++ |
| 3 | E292H | 8.848 | ++ |
| 4 | F291M | 6.242 | ++ |
| 5 | T41M | 0.211 | + |
| 6 | K50I | 4.063 | ++ |
| 7 | T375A | 0.381 | + |
| 8 | L74F | 0.799 | + |
| 9 | L246I | 3.152 | ++ |
| 10 | P598D | 0.662 | + |
| 11 | K573T | 0.267 | + |
| 12 | G480L | 0.19 | + |
| 13 | S219T | 3.206 | ++ |
| 14 | G191A | 3.848 | ++ |
| 15 | M148L | 1.748 | + |
| 16 | I380F | −0.467 | − |
| 17 | G193A | 3.732 | ++ |
| 18 | S110M | 0.019 | + |
| 19 | Q346V | 1.604 | + |
| 20 | I507Y | −0.075 | − |
| 21 | W259L | −0.353 | − |
| 22 | L363D | −0.167 | − |
| 23 | F425H | 0.806 | + |
| 24 | I212L | 0.299 | + |
| 25 | K285R | 2.634 | ++ |
| 26 | V481P | 7.068 | ++ |
| 27 | Y339F | 0.664 | + |
| 28 | D221T | 2.151 | ++ |
| 29 | V262C | 0.929 | + |
| 30 | I570V | 7.986 | ++ |
| 31 | S94R | 10.544 | ++ |
| 32 | L279V | 0.111 | + |
| 33 | L279M | 6.489 | ++ |
| 34 | L565M | 9.531 | ++ |
| 35 | N308T | −0.195 | − |
| 36 | R61P | 2.954 | ++ |
| 37 | A399M | −0.195 | − |
| 38 | S351F | −0.162 | − |
| 39 | S110F | −1.459 | − |
| 40 | L336T | 18.672 | ++ |
| 41 | T294I | −0.079 | − |
| 42 | E569M | 0.327 | + |
| 43 | H568G | 7.007 | ++ |
| 44 | L363V | 5.386 | ++ |
| 45 | A242V | 5.15 | ++ |
| 46 | Q236A | −0.231 | − |
| 47 | Y71F | 2.549 | ++ |
| 48 | N308A | −1.446 | − |
| 49 | L336I | 1.021 | + |
| 50 | V104L | 1.985 | + |
| 51 | S278C | 0.177 | + |
| 52 | I120M | 2.867 | ++ |
| 53 | A446M | 3.518 | ++ |
| 54 | K566A | 1.718 | + |
| 55 | W349M | −0.936 | − |
| 56 | V121T | 0.836 | + |
| 57 | D255A | −0.167 | − |
| 58 | T294L | −0.118 | − |
| 59 | A147S | 4.247 | ++ |
| 60 | N577Y | −0.127 | − |
| 61 | A367T | 1.816 | + |
| 62 | F245M | 1.259 | + |
| 63 | A113T | −0.8 | − |
| 64 | M542Y | 3.64 | ++ |
| 65 | E333S | 3.238 | ++ |
| 66 | K573S | 0.509 | + |
| 67 | G574M | 2.057 | ++ |
| 68 | T473S | 4.414 | ++ |
| 69 | T227L | −0.317 | − |
| 70 | V518I | 3.759 | ++ |
| 71 | N268M | 0.368 | + |
| 72 | L86A | 1.135 | + |
| 73 | Q114M | −0.292 | − |
| 74 | D255T | 0.48 | + |
| 75 | V78A | 2.026 | ++ |
| 76 | Y127F | 0.291 | + |
| 77 | L 565I | 4.241 | ++ |
| 78 | M272C | 0.224 | + |
| 79 | F469L | 0.231 | + |
| 80 | D567K | 0.911 | + |
| 81 | R414W | −0.169 | − |
| 82 | T583D | 1.122 | + |
| 83 | Q238M | 3.379 | ++ |
| 84 | T379C | 3.432 | ++ |
| 85 | L213I | 1.287 | + |
| 86 | W66G | −0.189 | − |
| 87 | M271A | 0.015 | + |
| 88 | P22D | 2.973 | ++ |
| 89 | D406E | 0.61 | + |
| 90 | G265M | 0.58 | + |
| 91 | V394R | −0.14 | − |
| 92 | R591Q | 1.092 | + |
| 93 | L352I | 2.171 | ++ |
| 94 | R252H | 0.533 | + |
| 95 | R296D | 0.978 | + |
| 96 | I281F | −0.634 | − |
| 97 | V311A | 3.581 | ++ |
| 98 | W256F | 0.128 | + |
| 99 | I578Q | 0.934 | + |
| 100 | T521P | 3.688 | ++ |
| 101 | T227D | −1.101 | − |
| 102 | V465L | 8.818 | ++ |
| 103 | F449I | −0.367 | − |
| 104 | G480I | −0.622 | − |
| 105 | P563F | 0.056 | + |
| 106 | L137M | 0.308 | + |
| 107 | G224A | 0.672 | + |
| 108 | S362H | 3.328 | ++ |
| 109 | S159R | 2.598 | ++ |
| 110 | W256V | 0.241 | + |
| 111 | V311R | 1.635 | + |
| 112 | I380C | −0.323 | − |
| 113 | G290A | 2.52 | ++ |
| 114 | Y127W | 2.826 | ++ |
| 115 | Q346A | 0.569 | + |
| 116 | G508V | 0.026 | + |
| 117 | V97A | 1.412 | + |
| 118 | A378C | −0.26 | − |
| 119 | D567Q | 3.473 | ++ |
| 120 | A113C | 5.546 | ++ |
| 121 | C411D | −0.015 | − |
| 122 | L396F | −0.483 | − |
| 123 | A173V | 2.366 | ++ |
| 124 | T473N | 7.614 | ++ |
| 125 | H201A | 3.259 | ++ |
| 126 | I527L | 4.367 | ++ |
| 127 | N270M | 0.138 | + |
| 128 | N283E | 5.507 | ++ |
| 129 | S368N | 1.905 | + |
| 130 | A124Y | 0.155 | + |
| 131 | A274Y | 0.116 | + |
| 132 | H502P | −0.425 | − |
| 133 | P286D | 0.55 | + |
| 134 | D255N | 0.213 | + |
| 135 | S575A | −0.058 | − |
| 136 | W476N | 1.998 | + |
| 137 | A43S | 2.744 | ++ |
| 138 | M272V | 1.548 | + |
| 139 | Q236C | −0.42 | − |
| 140 | H267I | −0.969 | − |
| 141 | K145G | 1.447 | + |
| 142 | I511V | 2.59 | ++ |
| 143 | V460Y | 1.723 | + |
| 144 | V16C | 0.656 | + |
| 145 | L305W | −0.627 | − |
| 146 | D282P | −0.364 | − |
| 147 | F435I | −0.803 | − |
| 148 | A367C | 1.523 | + |
| 149 | P377G | 0.254 | + |
| 150 | F214Y | 7.304 | ++ |
| 151 | F245L | 1.556 | + |
| 152 | M148V | 3.323 | ++ |
| 153 | L209I | 3.884 | ++ |
| 154 | I120F | 3.683 | ++ |
| 155 | P455E | −1.488 | − |
| 156 | M366I | 1.058 | + |
| 157 | G8P | 12.155 | ++ |
| 158 | S266N | 3.826 | ++ |
| 159 | K50F | 2.392 | ++ |
| 160 | G284F | −1.855 | − |
| 161 | R136E | 3.598 | ++ |
| 162 | K430A | 4.386 | ++ |
| 163 | A314Q | 9.424 | ++ |
| 164 | L363I | 2.323 | ++ |
| 165 | S362L | 1.03 | + |
| 166 | L243I | 0.581 | + |
| 167 | R230H | 3.955 | ++ |
| 168 | E10K | 0.765 | + |
| 169 | D462I | 0.156 | + |
| 170 | N275G | −1.076 | − |
| 171 | N419H | 1.248 | + |
| 172 | L365M | 3.576 | ++ |
| 173 | S20H | 4.706 | ++ |
| 174 | V460F | 4.939 | ++ |
| 175 | L273M | 2.365 | ++ |
| 176 | A434T | 0.409 | + |
| 177 | D485A | 3.431 | ++ |
| 178 | L243F | 6.762 | ++ |
| 179 | V140M | 4.193 | ++ |
| 180 | L12A | 8.444 | ++ |
| 181 | M272P | 0.307 | + |
| 182 | V262T | 2.512 | ++ |
| 183 | M421T | 1.799 | + |
| 184 | A234S | 5.314 | ++ |
| 185 | F291V | 3.238 | ++ |
| 186 | F291L | 4.715 | ++ |
| 187 | V37C | 1.894 | + |
| 188 | A107V | −0.386 | − |
| 189 | Q165R | 0.836 | + |
| 190 | S266T | −0.155 | − |
| 191 | N268A | 0.339 | + |
| 192 | S343P | 1.662 | + |
| 193 | V498C | 1.212 | + |
| 194 | V16L | −1.152 | − |
| 195 | S278T | 4.106 | ++ |
| 196 | A453G | 4.576 | ++ |
| 197 | M357W | 2.065 | ++ |
| 198 | L213C | −0.135 | − |
| 199 | V382L | 1.258 | + |
| 200 | A107P | −0.302 | − |
| 201 | S351R | 5.591 | ++ |
| 202 | L336M | 6.381 | ++ |
| 203 | H201P | −0.47 | − |
| 204 | S560D | 0.531 | + |
| 205 | E142Q | 4.045 | ++ |
| 206 | F269L | −0.152 | − |
| 207 | S266M | 0.296 | + |
| 208 | M272A | 3.266 | ++ |
| 209 | P302Q | 3.683 | ++ |
| 210 | V391S | 0.213 | + |
| 211 | G63D | 4.349 | ++ |
| 212 | I380H | 0.341 | + |
| 213 | L471T | 2.169 | ++ |
| 214 | 1392V | 0.662 | + |
| 215 | V151C | 4.339 | ++ |
| 216 | D468M | 0.284 | + |
| 217 | G303V | 0.434 | + |
| 218 | T287P | 0.078 | + |
| 219 | G277A | 0.497 | + |
| 220 | N260H | 4.536 | ++ |
| 221 | S532R | 5.196 | ++ |
| 222 | S128R | 0.085 | + |
| 223 | M357G | 2.773 | ++ |
| 224 | D255L | −1.53 | − |
| 225 | E359H | −0.194 | − |
| 226 | R296Q | 2.485 | ++ |
| 227 | T375C | 2.748 | ++ |
| 228 | S368Q | −0.152 | − |
| 229 | K566E | 0.924 | + |
| 230 | H383D | −0.394 | − |
| 231 | V382N | 1.621 | + |
| 232 | S351Q | 4.165 | ++ |
| 233 | L75D | 0.894 | + |
| 234 | M542W | 1.916 | + |
| 235 | D462T | −0.485 | − |
| 236 | N308S | −0.362 | − |
| 237 | W256I | −0.027 | − |
| 238 | M357V | 2.97 | ++ |
| 239 | Y115A | −3.789 | − |
| 240 | C233L | 0.174 | − |
| 241 | A432K | 5.485 | ++ |
| 242 | S369G | 1.809 | + |
| 243 | M409Q | 1.723 | + |
| 244 | W476Y | 11.76 | ++ |
| 245 | D452P | 4.781 | ++ |
| 246 | S266V | −0.495 | − |
| 247 | L243M | 3.701 | ++ |
| 248 | L12I | 1.411 | + |
| 249 | D567T | 1.25 | + |
| 250 | M357L | 2.705 | ++ |
| 251 | A274R | 0.303 | + |
| 252 | M315R | 2.0 | ++ |
| 253 | D241A | 1.182 | + |
| 254 | T41L | −0.215 | − |
| 255 | T227H | −0.076 | − |
| 256 | G83S | 1.591 | + |
| 257 | V225A | −0.422 | − |
| 258 | P286F | 0.089 | + |
| 259 | K299A | 0.657 | + |
| 260 | R230K | 3.239 | ++ |
| 261 | R61E | −0.02 | − |
| 262 | G264A | 0.163 | + |
| 263 | W349F | 2.275 | ++ |
| 264 | H267G | 0.226 | + |
| 265 | A84Y | 2.843 | ++ |
| 266 | G303I | −0.299 | − |
| 267 | P119V | 1.446 | + |
| 268 | H192T | 0.662 | + |
| 269 | W256C | −0.26 | − |
| 270 | N419S | 0.065 | + |
| 271 | R67K | 4.623 | ++ |
| 272 | G458N | −2.338 | − |
| 273 | W349L | 0.085 | + |
| 274 | S216G | −0.495 | − |
| 275 | K416R | 2.791 | ++ |
| 276 | A479P | 3.633 | ++ |
| 277 | T237A | 4.74 | ++ |
| 278 | F435M | 0.085 | + |
| 279 | P131E | 2.147 | ++ |
| 280 | L123M | 1.822 | + |
| 281 | L254I | 2.549 | ++ |
| 282 | V442K | 5.735 | ++ |
| 283 | D241H | 1.538 | + |
| 284 | G458F | −0.088 | − |
| 285 | N270H | −0.068 | − |
| 286 | G341A | −0.382 | − |
| 287 | E353R | 4.919 | ++ |
| 288 | S240A | 1.745 | + |
| 289 | I337L | 9.076 | ++ |
| 290 | R244E | 0.206 | + |
| 291 | N283D | 1.811 | + |
| 292 | P563L | 1.216 | + |
| 293 | L316W | −0.509 | − |
| 294 | K247R | 3.681 | ++ |
| 295 | L471V | 0.542 | + |
| 296 | L141M | 3.721 | ++ |
| 297 | D93G | 3.381 | ++ |
| 298 | G277C | −3.299 | − |
| 299 | V401M | 0.177 | + |
| 300 | L254F | −0.711 | − |
| 301 | I420V | 3.633 | ++ |
| 302 | R540Q | 4.132 | ++ |
| 303 | V447A | 0.449 | + |
| 304 | I58V | 2.289 | ++ |
| 305 | H568N | 2.353 | ++ |
| 306 | A6L | 14.015 | ++ |
| 307 | V382C | 3.315 | ++ |
| 308 | K50R | 6.485 | ++ |
| 309 | V394K | 0.164 | + |
| 310 | I120V | 3.716 | ++ |
| 311 | E359Q | 3.069 | ++ |
| 312 | G387D | 2.336 | ++ |
| 313 | K50V | 3.373 | ++ |
| 314 | E400D | 1.528 | + |
| 315 | A399V | 6.784 | ++ |
| 316 | L109V | 2.682 | ++ |
| 317 | T215S | 0.01 | + |
| 318 | S575Y | 0.194 | + |
| 319 | S351W | 0.294 | + |
| 320 | V526L | 3.176 | ++ |
| 321 | D282M | −0.029 | − |
| 322 | F338W | 0.215 | + |
| 323 | D5R | 5.304 | ++ |
| 324 | M271P | −0.32 | − |
| 325 | M357P | −0.397 | − |
| 326 | D255I | −2.221 | − |
| 327 | R464D | −0.456 | − |
| 328 | P515D | 11.23 | ++ |
| 329 | L404V | 7.76 | ++ |
| 330 | G265L | −0.083 | − |
| 331 | L305Y | −0.178 | − |
| 332 | V401I | 3.49 | ++ |
| 333 | C411S | 1.276 | + |
| 334 | V460I | 3.392 | ++ |
| 335 | H106W | −0.236 | − |
| 336 | S20K | 4.363 | ++ |
| 337 | S456D | 3.428 | ++ |
| 338 | D221M | 1.853 | + |
| 339 | A446I | 3.141 | ++ |
| 340 | N318H | 2.745 | ++ |
| 341 | Q346P | 0.342 | + |
| 342 | S110Y | −0.127 | − |
| 343 | F269M | −0.381 | − |
| 344 | I337M | 1.735 | + |
| 345 | P53L | 0.364 | + |
| 346 | N80K | 1.971 | + |
| 347 | F510A | 0.025 | + |
| 348 | L316M | 1.41 | + |
| 349 | D587A | 6.108 | ++ |
| 350 | H383N | −0.724 | − |
| 351 | V181E | 2.908 | ++ |
| 352 | Y330L | 0.165 | − |
| 353 | Q126M | 1.981 | + |
| 354 | K285F | 0.47 | + |
| 355 | S519T | 6.044 | ++ |
| 356 | G191E | 4.633 | ++ |
| 357 | G205T | 4.184 | ++ |
| 358 | L565V | 5.646 | ++ |
| 359 | I253Y | 0.766 | − |
| 360 | V460R | 2.477 | ++ |
| 361 | T373S | −0.008 | − |
| 362 | R322Q | 3.164 | ++ |
| 363 | F461I | 0.291 | − |
| 364 | E125P | 8.436 | ++ |
| 365 | D255G | −0.607 | − |
| 366 | S466D | 2.846 | ++ |
| 367 | D567N | 3.076 | ++ |
| 368 | N577A | 2.323 | ++ |
| 369 | Q114W | −0.065 | − |
| 370 | S368E | −0.077 | − |
| 371 | Q194S | 2.161 | ++ |
| 372 | C411V | −0.851 | − |
| 373 | L305V | 8.711 | ++ |
| 374 | S466K | 0.965 | + |
| 375 | V442H | 3.441 | ++ |
| 376 | G480S | 4.648 | ++ |
| 377 | N554E | 7.162 | ++ |
| 378 | M366V | 0.184 | + |
| 379 | M357I | 5.973 | ++ |
| 380 | S110A | 1.89 | + |
| 381 | P251E | −0.659 | − |
| 382 | V394I | 1.619 | + |
| 383 | V391T | 0.121 | + |
| 384 | Q236I | −0.212 | − |
| 385 | Q478T | 1.81 | + |
| 386 | Y313L | −0.386 | − |
| 387 | V348T | 2.794 | ++ |
| 388 | F269I | −0.906 | − |
| 389 | F263A | −1.138 | − |
| 390 | P39R | 2.997 | ++ |
| 391 | A274V | −0.322 | − |
| 392 | A306V | 0.587 | + |
| 393 | L243W | 13.899 | ++ |
| 394 | D15E | 3.817 | ++ |
| 395 | A274M | 0.535 | + |
| 396 | G387R | 7.902 | ++ |
| 397 | F461L | 0.651 | + |
| 398 | R483E | −0.966 | − |
| 399 | A446L | 8.209 | ++ |
| 400 | I337F | −0.073 | − |
| 401 | L295W | −0.121 | − |
| 402 | V311P | −0.697 | − |
| 403 | L243V | 0.022 | + |
| 404 | L295M | 5.248 | ++ |
| 405 | M381N | −0.478 | − |
| 406 | N308L | −0.471 | − |
| 407 | T208D | 0.884 | + |
| 408 | L334I | 5.177 | ++ |
| 409 | L12V | 2.41 | ++ |
| 410 | A434V | 4.976 | ++ |
| 411 | G474E | −0.427 | − |
| 412 | A242C | 11.143 | ++ |
| 413 | Q564D | 1.709 | + |
| 414 | I386D | −2.299 | − |
| 415 | V401C | 2.161 | ++ |
| 416 | M77R | 5.891 | ++ |
| 417 | G83H | −0.37 | − |
| 418 | D572R | −2.054 | − |
| 419 | W476L | 5.725 | ++ |
| 420 | S389A | 13.225 | ++ |
| 421 | Q236M | 0.281 | + |
| 422 | M372L | −0.982 | − |
| 423 | A113V | −0.661 | − |
| 424 | L161I | 4.773 | ++ |
| 425 | I281L | 0.379 | + |
| 426 | R322K | 5.578 | ++ |
| 427 | V116I | 6.737 | ++ |
| 428 | T379V | 4.82 | ++ |
| 429 | N268C | −0.844 | − |
| 430 | W44M | −0.086 | − |
| 431 | Y339T | 2.783 | ++ |
| 432 | I239F | −0.098 | − |
| 433 | M381F | −1.43 | − |
| 434 | I578A | 1.63 | + |
| 435 | V481E | 5.864 | ++ |
| 436 | L226M | 2.308 | ++ |
| 437 | H267M | 7.252 | ++ |
| 438 | L163V | 3.268 | ++ |
| 439 | T176D | 4.313 | ++ |
| 440 | V169I | 4.117 | ++ |
| 441 | A187D | 6.599 | ++ |
| 442 | E125T | 6.335 | ++ |
| 443 | D485L | 6.272 | ++ |
| 444 | W370Y | 0.311 | + |
| 445 | W48P | −0.476 | − |
| 446 | H267W | 0.327 | + |
| 447 | E170I | 2.735 | ++ |
| 448 | H69F | 6.593 | ++ |
| 449 | L226I | 1.668 | + |
| 450 | I380S | 0.976 | + |
| 451 | C233V | −0.423 | − |
| 452 | C233M | −0.401 | − |
| 453 | I212M | 0.035 | + |
| 454 | K145R | 4.596 | ++ |
| 455 | I392C | −0.243 | − |
| 456 | F435L | −0.358 | − |
| 457 | D221L | 2.893 | ++ |
| 458 | N235Q | 0.087 | + |
| 459 | G284R | −0.65 | − |
| 460 | H383R | 0.314 | + |
| 461 | A124S | 7.846 | ++ |
| 462 | A306Y | 0.325 | + |
| 463 | L365V | 0.013 | + |
| 464 | D255H | −0.101 | − |
| 465 | G341S | −0.585 | − |
| 466 | G101P | 0.22 | + |
| 467 | Q114L | −2.952 | − |
| 468 | Y313F | 4.582 | ++ |
| 469 | M315A | 4.776 | ++ |
| 470 | A479V | 4.591 | ++ |
| 471 | N260S | 21.728 | ++ |
| 472 | K211L | 0.031 | + |
| 473 | P418D | −1.565 | − |
| 474 | G312I | −0.224 | − |
| 475 | M357F | 8.395 | ++ |
| 476 | V394Q | −0.01 | − |
| 477 | A274C | 8.014 | ++ |
| 478 | I507V | 6.381 | ++ |
| 479 | V382T | 4.152 | ++ |
| 480 | Y127L | 8.946 | ++ |
| 481 | I386V | 8.564 | ++ |
| 482 | R414L | 0.958 | + |
| 483 | F338L | 6.678 | ++ |
| 484 | E384P | −0.475 | − |
| 485 | N283P | 1.138 | + |
| 486 | N76T | 6.37 | ++ |
| 487 | N268G | −0.216 | − |
| 488 | L28M | 2.103 | ++ |
| 489 | N275C | 0.604 | + |
| 490 | Q346E | 2.645 | ++ |
| 491 | L246T | −0.314 | − |
| 492 | S343A | 1.233 | + |
| 493 | F269V | −0.068 | − |
| 494 | I558F | −0.389 | − |
| 495 | A342S | 0.476 | + |
| 496 | I386L | 0.912 | + |
| 497 | H267F | 5.102 | ++ |
| 498 | L344M | 2.459 | ++ |
| 499 | Q238S | −0.667 | − |
| 500 | L12M | 2.031 | ++ |
| 501 | P405D | −0.449 | − |
| 502 | F307P | 0.047 | + |
| 503 | V450D | 0.122 | + |
| 504 | S20R | 3.767 | ++ |
| 505 | G480P | 2.408 | ++ |
| 506 | L279I | 1.907 | + |
| 507 | R585H | 0.228 | + |
| 508 | V394L | −4.131 | − |
| 509 | T237M | 0.163 | + |
| 510 | L365I | 1.104 | + |
| 511 | L123V | 1.247 | + |
| 512 | C233W | −4.208 | − |
| 513 | S159E | 3.935 | ++ |
| 514 | M357A | 2.786 | ++ |
| 515 | A234W | −1.331 | − |
| 516 | F307W | 0.336 | + |
| 517 | M232L | 6.857 | ++ |
| 518 | I253M | 0.455 | + |
| 519 | C233A | 0.611 | + |
| 520 | R414V | −0.554 | − |
| 521 | F263Y | 4.928 | ++ |
| 522 | D282E | −0.13 | − |
| 523 | L305I | 2.072 | ++ |
| 524 | D567S | 4.644 | ++ |
| 525 | Q238W | −1.578 | − |
| 526 | T227I | 0.08 | + |
| 527 | N235S | −2.081 | − |
| 528 | A376T | −0.364 | − |
| 529 | G303A | 6.156 | ++ |
| 530 | I507W | −0.035 | − |
| 531 | P395A | −1.252 | − |
| 532 | F331G | 0.22 | + |
| 533 | N76D | 8.585 | ++ |
| 534 | L396I | −0.051 | − |
| 535 | E358Q | 13.593 | ++ |
| 536 | G103R | 0.336 | + |
| 537 | S456E | −2.593 | − |
| 538 | N577L | 0.108 | + |
| 539 | L123I | 8.492 | ++ |
| 540 | A57F | 2.959 | ++ |
| 541 | Q126N | 5.694 | ++ |
| 542 | A376S | 2.211 | ++ |
| 543 | F291P | −0.141 | − |
| 544 | L344I | 7.989 | ++ |
| 545 | Q114G | −0.206 | − |
| 546 | K211T | −1.268 | − |
| 547 | P119T | 2.862 | ++ |
| 548 | L213V | 0.458 | + |
| 549 | E467K | 0.664 | + |
| 550 | P385G | 1.18 | + |
| 551 | F307Y | 0.142 | − |
| 552 | L413I | −0.274 | − |
| 553 | M409E | 1.463 | + |
| 554 | S466A | 6.422 | ++ |
| 555 | G387N | 5.216 | ++ |
| 556 | M381L | 1.356 | + |
| 557 | I386T | 3.738 | ++ |
| 558 | H14P | 5.859 | ++ |
| 559 | F338M | 6.509 | ++ |
| 560 | D221R | 1.978 | + |
| 561 | P563W | −0.664 | − |
| 562 | K211C | −0.748 | − |
| 563 | F338V | 2.188 | ++ |
| 564 | L273V | 2.956 | ++ |
| 565 | Q564K | 0.435 | + |
| 566 | S369A | 4.348 | ++ |
| 567 | D485E | 5.372 | ++ |
| 568 | S110D | −1.879 | − |
| 569 | N308G | 0.381 | + |
| 570 | L45I | 3.735 | ++ |
| 571 | G265W | −0.103 | − |
| 572 | I120A | 2.596 | ++ |
| 573 | R414C | 0.534 | + |
| 574 | N275S | −4.136 | − |
| 575 | R388K | 4.723 | ++ |
| 576 | H106F | 0.05 | + |
| 577 | I281V | 3.767 | ++ |
| 578 | K285W | −0.967 | − |
| 579 | I239M | 0.451 | + |
| 580 | R584N | 8.063 | ++ |
| 581 | H69Y | 4.935 | ++ |
| 582 | A306I | 1.985 | + |
| 583 | M366L | 0.583 | + |
| 584 | M148A | 1.731 | + |
| 585 | L334M | 0.615 | + |
| 586 | P119I | 0.929 | + |
| 587 | D255C | 0.881 | + |
| 588 | K211M | −0.281 | − |
| 589 | A378T | 0.585 | + |
| 590 | M232I | −2.704 | − |
| 591 | E300Q | 3.509 | ++ |
| 592 | G492P | 3.489 | ++ |
| 593 | R414Q | −0.664 | − |
| 594 | A59V | 1.267 | + |
| 595 | S532H | 3.731 | ++ |
| 596 | R322A | 6.244 | ++ |
| 597 | N76A | 4.369 | ++ |
| 598 | L226V | 2.082 | ++ |
| 599 | N419Q | 2.375 | ++ |
| 600 | T443S | 0.564 | + |
| 601 | G284W | −0.15 | − |
| 602 | V348I | 18.586 | ++ |
| 603 | Y339S | 1.49 | + |
| 604 | H14A | 1.513 | + |
| 605 | L213Q | −3.533 | − |
| 606 | I298M | 4.273 | ++ |
| 607 | G312M | 0.782 | + |
| 608 | L209T | 1.692 | + |
| 609 | Y330F | 3.885 | ++ |
| 610 | I298V | 4.07 | ++ |
| 611 | I253F | −0.244 | − |
| 612 | W476F | 9.919 | ++ |
| 613 | A306M | −0.17 | − |
| 614 | Y339C | −0.867 | − |
| 615 | L441F | 2.275 | ++ |
| 616 | R244P | 15.519 | ++ |
| 617 | F245H | −0.133 | − |
| 618 | N260Y | 0.378 | + |
| 619 | H568D | 3.193 | ++ |
| 620 | G83Q | −0.12 | − |
| 621 | M231N | −0.511 | − |
| 622 | F355A | 3.028 | ++ |
| 623 | E400Q | 5.849 | ++ |
| 624 | A306L | −0.664 | − |
| 625 | I120L | 3.111 | ++ |
| 626 | L459Y | −0.209 | − |
| 627 | I239V | 3.103 | ++ |
| 628 | K50L | 4.77 | ++ |
| 629 | M272T | 0.962 | + |
| 630 | W349A | −0.434 | − |
| 631 | N275H | 1.872 | + |
| 632 | D221K | 3.047 | ++ |
| 633 | I380M | −0.444 | − |
| 634 | R322H | 4.64 | ++ |
| 635 | D496H | −1.744 | − |
| 636 | K247P | 0.711 | + |
| 637 | I253L | 8.06 | ++ |
| 638 | N76R | 4.322 | ++ |
| 639 | T379I | 0.64 | + |
| 640 | L305F | −1.316 | − |
| 641 | L45V | 1.139 | + |
| 642 | V348L | 3.964 | ++ |
| 643 | A107I | −0.12 | − |
| 644 | L316I | 2.631 | ++ |
| 645 | S218T | 2.114 | ++ |
| 646 | N595E | 5.493 | ++ |
| 647 | G83N | 0.941 | + |
| 648 | M232V | −0.551 | − |
| 649 | P455G | −1.046 | − |
| 650 | I337A | −0.284 | − |
| 651 | L189V | 1.892 | + |
| 652 | V262A | 0.018 | + |
| 653 | K566D | 2.756 | ++ |
| 654 | L336Y | 2.312 | ++ |
| 655 | G168H | 4.498 | ++ |
| 656 | A367M | 3.043 | ++ |
| 657 | P222S | 0.794 | + |
| 658 | T41V | 4.338 | ++ |
| 659 | V16I | 2.434 | ++ |
| 660 | L396M | −2.372 | − |
| 661 | A92K | 5.419 | ++ |
| 662 | V262I | −0.628 | − |
| 663 | A367I | 9.206 | ++ |
| 664 | D15L | −1.688 | − |
| 665 | L254V | 2.15 | ++ |
| 666 | L344V | 0.709 | + |
| 667 | I120W | −1.55 | − |
| 668 | V442Y | 9.453 | ++ |
| 669 | S368T | −1.541 | − |
| 670 | L336V | 87.047 | ++ |
| 671 | A185S | 4.373 | ++ |
| 672 | G417S | −0.306 | − |
| 673 | N268V | −1.262 | − |
| 674 | V401L | 4.208 | ++ |
| 675 | P13C | 1.831 | + |
| 676 | S278A | 3.621 | ++ |
| 677 | T237Q | 0.142 | + |
| 678 | F150V | 0.024 | + |
| 679 | P385D | −0.889 | − |
| 680 | W349I | −0.869 | − |
| 681 | L396V | 1.333 | + |
| 682 | I380T | 0.037 | + |
| 683 | I337V | 4.498 | ++ |
| 684 | W259F | −0.391 | − |
| 685 | S575K | −0.178 | − |
| 686 | G398D | −0.64 | − |
| 687 | H267L | 1.517 | + |
| 688 | L190V | 3.97 | ++ |
| 689 | T287A | 0.671 | + |
| 690 | E125W | 2.209 | ++ |
| 691 | H267V | −2.865 | − |
| 692 | D462V | 1.393 | + |
| 693 | A367V | 12.66 | ++ |
| 694 | C411G | −1.56 | − |
| 695 | D177E | 9.598 | ++ |
| 696 | E384N | 2.533 | ++ |
| 697 | A446V | 5.667 | ++ |
| 698 | S389P | 6.875 | ++ |
| 699 | K211V | −0.752 | − |
| 700 | L209P | −0.171 | − |
| 701 | R591E | 0.16 | + |
| 702 | S389L | 2.52 | ++ |
| 703 | A340G | 8.676 | ++ |
| 704 | E51R | 1.297 | + |
| 705 | A399T | 8.231 | ++ |
| 706 | E19D | 3.474 | ++ |
| 707 | I253V | 2.684 | ++ |
| 708 | K285D | 0.086 | + |
| 709 | F291I | 6.08 | ++ |
| 710 | S278H | −0.942 | − |
| 711 | L213M | 1.156 | + |
| 712 | V450M | 4.22 | ++ |
| 713 | V442R | 8.604 | ++ |
| 714 | D572P | 5.468 | ++ |
| 715 | I130M | 4.747 | ++ |
| 716 | T237R | −0.406 | − |
| 717 | C411N | −0.534 | − |
| 718 | N318D | 3.212 | ++ |
| 719 | L490C | 2.064 | ++ |
| 720 | R136Q | 0.427 | + |
| 721 | G480A | 4.124 | ++ |
| 722 | N40T | 1.818 | + |
| 723 | E433R | 6.617 | ++ |
| 724 | F425L | 1.26 | + |
| 725 | A242T | 3.495 | ++ |
| 726 | Y313W | 1.078 | + |
| 727 | G265F | −1.493 | − |
| 728 | F307H | −0.006 | − |
| 729 | R136K | 2.93 | ++ |
| 730 | D347E | 2.065 | ++ |
| 731 | V497A | −1.548 | − |
| 732 | R88L | 1.711 | + |
| 733 | I511L | 0.953 | + |
| 734 | H14Q | 19.193 | ++ |
| 735 | M315K | 3.104 | ++ |
| 736 | K211A | −2.289 | − |
| 737 | E10D | 6.616 | ++ |
| 738 | F263H | −0.837 | − |
| 739 | V401A | 1.154 | + |
| 740 | L493F | −1.489 | − |
| 741 | H267C | −0.48 | − |
| 742 | Q126R | 34.63 | ++ |
| 743 | M421F | 3.219 | ++ |
| 744 | V140C | 2.956 | ++ |
| 745 | E10P | 2 | ++ |
| 746 | V140I | 3.726 | ++ |
| 747 | A242W | −0.079 | − |
| 748 | S25N | 1.667 | + |
| 749 | E170V | 2.045 | ++ |
| 750 | E321R | 5.189 | ++ |
| 751 | P122A | −0.582 | − |
| 752 | G387H | 7.716 | ++ |
| 753 | R585L | −1.736 | − |
| 754 | A62D | 5.209 | ++ |
| 755 | A87R | 3.716 | ++ |
| 756 | Q238N | −1.226 | − |
| 757 | F461Y | 5.593 | ++ |
| 758 | V442C | 4.896 | ++ |
| 759 | L336F | 0.407 | + |
| 760 | D485R | 4.822 | ++ |
| 761 | R244Q | 4.681 | ++ |
| 762 | L413V | 0.807 | − |
| 763 | Q564S | 6.588 | ++ |
| 764 | Y157L | −3.03 | − |
| 765 | V311T | 3.907 | ++ |
| 766 | N308V | −1.015 | − |
| 767 | E569K | 0.078 | + |
| 768 | L246M | −1.074 | − |
| 769 | D335R | −0.105 | − |
| 770 | G398H | 0.433 | + |
| 771 | W476V | 7.139 | ++ |
| 772 | T287V | 0.075 | + |
| 773 | C233I | −0.104 | − |
| 774 | E10N | 5.07 | ++ |
| 775 | Q114A | −1.199 | − |
| 776 | G303P | 4.99 | ++ |
| 777 | I120Y | 3.993 | ++ |
| 778 | T294A | 0.105 | + |
| 779 | L257M | −0.392 | − |
| 780 | E433D | 5.385 | ++ |
| 781 | Y593A | 0.065 | + |
| 782 | G516A | 6.185 | ++ |
| 783 | L85F | 2.118 | ++ |
| 784 | V415I | −2.319 | − |
| 785 | L305H | −0.471 | − |
| 786 | T304S | 4.081 | ++ |
| 787 | G312V | 0.797 | + |
| 788 | S220G | 7.104 | ++ |
| 789 | D567A | 6.131 | ++ |
| 790 | A376C | −0.452 | − |
| 791 | T237E | −0.824 | − |
| 792 | A376G | 13.05 | ++ |
| 793 | P22E | 4.572 | ++ |
| 794 | V104W | 0.353 | + |
| 795 | G303C | 1.587 | + |
| 796 | P164A | 4.91 | ++ |
| 797 | A399C | 12.396 | ++ |
| 798 | S456Q | −0.973 | − |
| 799 | I58L | 5.586 | ++ |
| 800 | E596K | 6.185 | ++ |
| 801 | R388H | 2.118 | ++ |
| 802 | A378V | −0.215 | − |
| 803 | L565P | 7.229 | ++ |
| 804 | V382P | 0.558 | + |
| 805 | A107L | −0.475 | − |
| 806 | I380V | 0.719 | + |
| 807 | T228S | 4.275 | ++ |
| 808 | N72A | 6.301 | ++ |
| 809 | L316F | −0.642 | − |
| 810 | Q564L | 1.935 | + |
| 811 | Q238H | 0.525 | + |
| 812 | P345S | 2.758 | ++ |
| 813 | S20D | 2.126 | ++ |
| 814 | L86Q | 4.901 | ++ |
| 815 | D255V | 0.721 | + |
| 816 | V460W | 1.91 | + |
| 817 | A149V | 6.018 | ++ |
| 818 | L295V | 26.198 | ++ |
| 819 | E321Q | 4.613 | ++ |
| 820 | A274H | 0.505 | + |
| 821 | I601L | 1.408 | + |
| 822 | W256Y | 0.458 | + |
| 823 | L295I | 8.958 | ++ |
| 824 | N283R | 0.837 | + |
| 825 | L273F | 2.258 | ++ |
| 826 | F245W | 0.039 | + |
| 827 | E170L | 2.542 | ++ |
| 828 | D335K | 0.875 | + |
| 829 | V447G | −0.288 | − |
| 830 | G196P | 1.608 | + |
| 831 | S362C | 3.83 | ++ |
| 832 | R252V | 1.892 | + |
| 833 | G574L | 0.837 | + |
| 834 | A314E | 0.175 | + |
| 835 | M381C | 0.601 | + |
| 836 | M372Q | −3.099 | − |
| 837 | G361F | −1.91 | − |
| 838 | W370F | −2.378 | − |
| 839 | S240R | −3.063 | − |
| 840 | S575T | 5.946 | ++ |
| 841 | V526M | 3.82 | ++ |
| 842 | S7K | 10.955 | ++ |
| 843 | Q478L | −0.432 | − |
| 844 | G265C | −0.442 | − |
| 845 | S7T | 5.608 | ++ |
| 846 | A124N | 2.758 | ++ |
| 847 | H383P | −0.497 | − |
| 848 | G387E | 1.492 | + |
| 849 | A107W | −1.175 | − |
| 850 | H69L | 5.696 | ++ |
| 851 | L352M | 1.358 | + |
| 852 | K566P | 0.616 | + |
| 853 | D451E | 1.975 | + |
| 854 | Q236V | −0.039 | − |
| 855 | F307M | 4.375 | ++ |
| 856 | E384D | 0.521 | + |
| 857 | G398N | −0.442 | − |
| 858 | D282A | −0.253 | − |
| 859 | 158M | 5.982 | ++ |
| 860 | N72R | 4.468 | ++ |
| 861 | L305M | 2.198 | ++ |
| 862 | S100C | 0.072 | + |
| 863 | L336W | 1.946 | + |
| 864 | I58F | 11.459 | ++ |
| 865 | L565T | 10.631 | ++ |
| 866 | I239C | −0.829 | − |
| 867 | E19R | 2.025 | ++ |
| 868 | L514N | 4.485 | ++ |
| 869 | F186Y | 3.508 | ++ |
| 870 | I535L | 0.449 | + |
| 871 | V262M | −0.358 | − |
| 872 | V81C | 4.199 | ++ |
| 873 | V98M | 3.342 | ++ |
| 874 | K145D | 1.542 | + |
| 875 | N419M | 0.225 | + |
| 876 | T41I | 1.242 | + |
| 877 | I108V | 5.058 | ++ |
| 878 | L441M | 1.441 | + |
| 879 | Q564T | 4.108 | ++ |
| 880 | M271C | 1.137 | + |
| 881 | M315L | 2.152 | ++ |
| 882 | I298L | 1.371 | + |
| 883 | D241E | 3.691 | ++ |
| 884 | A149L | −0.125 | − |
| 885 | P95R | 3.528 | ++ |
| 886 | A599S | 2.208 | ++ |
| 887 | R354K | 8.843 | ++ |
| 888 | G195S | 6.689 | ++ |
| 889 | D5E | 12.653 | ++ |
| 890 | K247E | −0.285 | − |
| 891 | F355R | −0.006 | − |
| 892 | D282H | −0.036 | − |
| 893 | L363G | 1.593 | + |
| 894 | M366F | 0.233 | + |
| 895 | E384W | 1.415 | + |
| 896 | M409G | 4.873 | ++ |
| 897 | L209V | 0.71 | + |
| 898 | F245Y | −0.016 | − |
| 899 | A546N | 2.954 | ++ |
| 900 | H267A | −0.732 | − |
| 901 | A84H | 1.014 | + |
| 902 | K470V | 0.256 | + |
| 903 | E167R | 8.515 | ++ |
| 904 | T379L | 2.522 | ++ |
| 905 | G480V | −0.846 | − |
| 906 | H201L | 3.403 | ++ |
| 907 | M381V | −3.327 | − |
| 908 | A399I | 9.686 | ++ |
| 909 | P455R | −2.962 | − |
| 910 | I570L | 1.523 | + |
| 911 | M271T | −0.092 | − |
| 912 | G458Y | −0.287 | − |
| 913 | Q564R | 1.144 | + |
| 914 | D15S | 3.962 | ++ |
| 915 | V183I | 2.043 | ++ |
| 916 | I239L | 1.295 | + |
| 917 | V104A | 3.342 | ++ |
| 918 | K402R | −0.431 | − |
| 919 | G458W | −2.483 | − |
| 920 | A314H | 4.757 | ++ |
| 921 | R244H | 0.925 | + |
| 922 | L352Y | −2.752 | − |
| 923 | K175R | 8.765 | ++ |
| 924 | T521W | 2.665 | ++ |
| 925 | R244G | 1.566 | + |
| 926 | D221H | 2.076 | ++ |
| 927 | A367L | 5.393 | ++ |
| 928 | E10R | 2.314 | ++ |
| 929 | D241Q | 5.317 | ++ |
| 930 | T379A | 1.176 | + |
| 931 | L588Q | 1.263 | + |
| 932 | N260F | 0.637 | + |
| 933 | L344C | 2.841 | ++ |
| 934 | A107F | −0.096 | − |
| 935 | N308M | −1.664 | − |
| 936 | L441Y | −0.595 | − |
| 937 | S456N | 10.678 | ++ |
| 938 | R504H | −0.058 | − |
| 939 | Y71W | 2.552 | ++ |
| 940 | P377M | −1.279 | − |
| 941 | A306F | −2.014 | − |
| 942 | N270Y | −1.154 | − |
| 943 | L273I | 4.552 | ++ |
| 944 | N270T | −1.283 | − |
| 945 | P515S | 4.323 | ++ |
| 946 | L254M | 6.301 | ++ |
| 947 | D255S | −1.441 | − |
| 948 | G463D | 0.114 | + |
| 949 | Q126A | 6.443 | ++ |
| 950 | I281M | −0.164 | − |
| 951 | M271L | 1.388 | + |
| 952 | L334V | 6.144 | ++ |
| 953 | F512I | 1.846 | + |
| 954 | W349Y | 3.08 | ++ |
| 955 | P513V | −1.219 | − |
| 956 | H383Y | 4.022 | ++ |
| 957 | D15R | −0.495 | − |
| 958 | E561D | 2.043 | ++ |
| 959 | D407P | 5.943 | ++ |
| 960 | A274W | −0.48 | − |
| 961 | R552Q | 1.828 | + |
| 962 | W476I | 5.254 | ++ |
| 963 | G312F | 1.928 | + |

Key

| | |
|---|---|
| − | lower activity than control <1 |
| + | similar activity to control ≥1 |
| ++ | higher activity than control >2 |

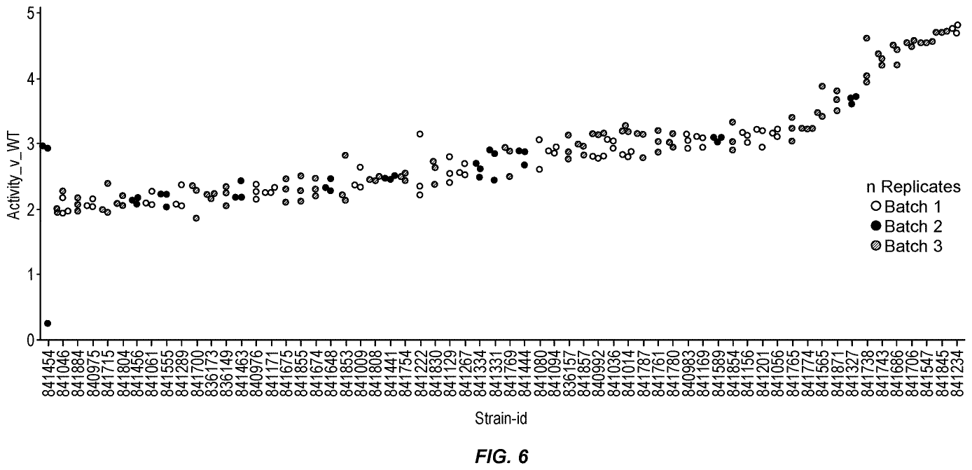

Variants from the homolog 1 subset yielded many improved variants, while the performance of the variants from the homolog 2 subset was overall poor. Because the homolog 2 template control also showed lower than expected performance compared to pilot experiments, a representative selection of 120 variants, including the 12 top performing variants, was included in the secondary screening. The 60 best homolog 1 variants were also promoted to secondary screening, the results of which are shown in FIG. 6.

### 3.2. Secondary Screen

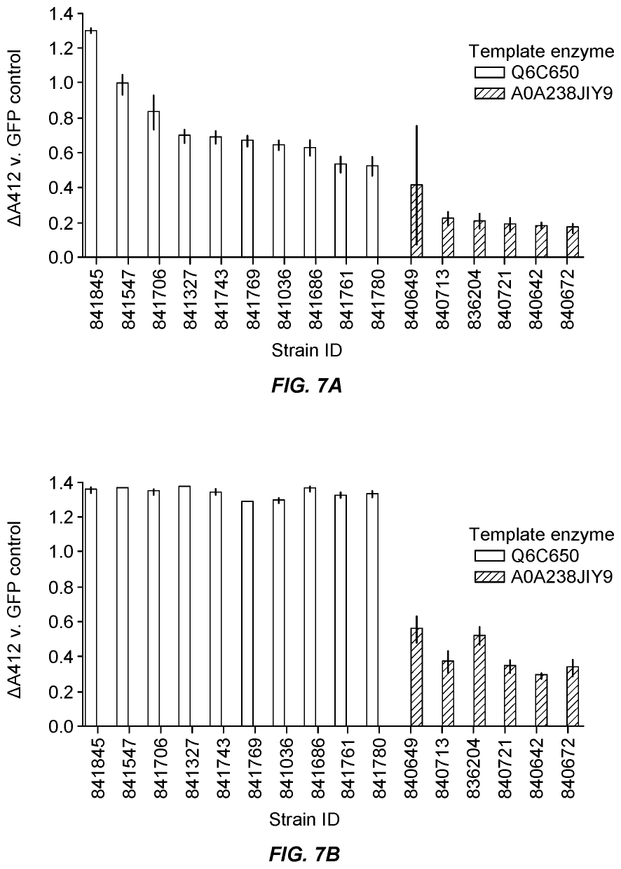

The cultures in the secondary screen went through a similar processes as the primary screen and enzymatic activity on HHA was assayed by measuring absorbance at 412 nm (DTNB assay) after 4 h or 1h. Top strains from the secondary screen are shown in FIGS. 7A and 7B. The genetic identity of these variants was confirmed by sequencing.

### Example 4. ALDH Template Library

### 4.1. Screening and Sourcing of Metagenomic Library ALDH Candidates and ALDH Template Library

Genes encoding ALDH candidate template enzymes (SEQ ID NO: 3; SEQ ID NO: 4; SEQ ID NO: 5; SEQ ID NO: 6; and SEQ ID NO: 7) were identified, sourced, synthesized, expressed and evaluated in an in vitro cell lysate assay as described in Example 1.1. Homolog 3—SEQ ID NO: 3 as set forth in TABLE 2 was selected as the template candidate.

### Example 5. Assaying the ALDH Template Library

### 5.1. In Vitro Cell Lysate Assay for Substrate Specificity

Based on the results from Example 4, the genes encoding the ALDH enzyme candidates were synthesized, expressed and evaluated for substrate specificity in an in vitro cell lysate assay as described in Example 2.1.

Results show the inverted rate of NADH consumption as a proxy for enzymatic activity (full data not shown). Homolog 3 from A. massiliensis displayed the highest activity on HHA-CoA (TABLE 10), but note that the rate obtained for this enzyme on adipyl-CoA is likely an underestimate due to the nature of the kinetic assay, in that at the start of measurement the NADH signal had already dropped considerably. This was not the case for all other enzyme-substrate combinations shown. Although a lower initial absorbance signal was not noted here for the homolog 3/HHA-CoA combination, subsequent screening performed at a higher dilution of lysate also indicated that activity on HHA-CoA is likewise higher relative to activity on succinyl-CoA and acetyl-CoA than would be suggested based on these results.

From this data, homolog 3 (SEQ ID NO: 3) was selected as template for the protein engineering library screen with the goal of selecting variants with (a) reduced activity on succinyl-CoA, and (b) conserved or improved activity on HHA-CoA and potentially adipyl-CoA.

### 5.2. Kinetic Characterization

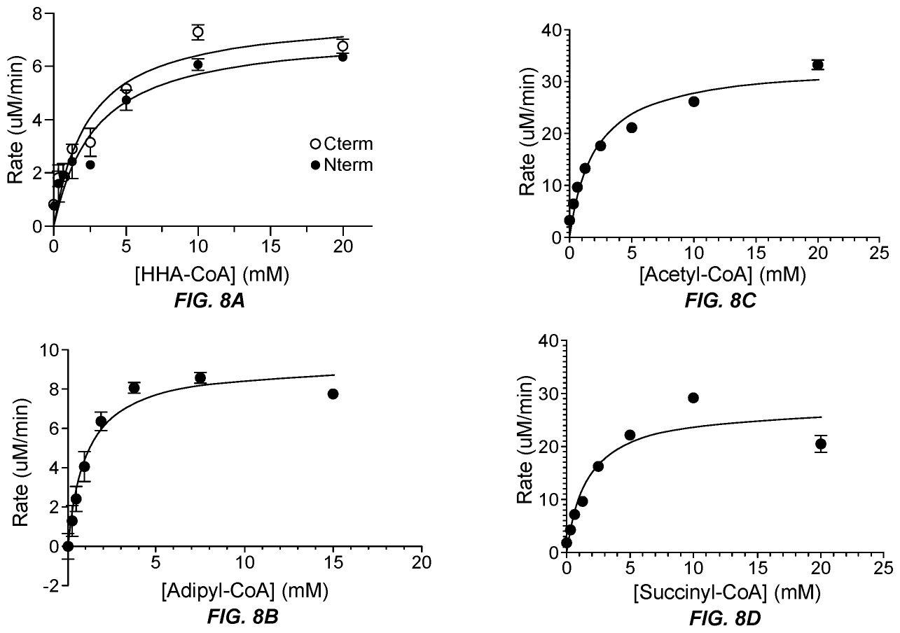

Next, the purification and biochemical characterization of homolog 3 (SEQ ID NO: 3) with the substrates HHA-CoA (FIG. 8A), adipyl-CoA (FIG. 8B), acetyl-CoA (FIG. 8C), and Succinyl-CoA (FIG. 8D) was established. Calculated kinetic parameters are listed in TABLE 11. In contrast to what earlier crude lysate data suggested, catalytic efficiency on adipyl-CoA and HHA-CoA are comparable to each other and an order of magnitude higher than with succinyl-CoA. Activity on adipyl-CoA can be desirable as it is part of the upstream HHA pathway.

TABLE 10
ALDH Template Candidates in vitro cell
lysate assay for substrate specificity

| | Rate (mA340/min) | | | |
|---|---|---|---|---|
| Homolog # | HHA- | Acetyl- | Adipyl- | Succinyl- |
| (Seq ID No) | CoA | CoA | CoA | CoA |
| 3 (SEQ ID NO: 2) | −18.2 | −1.10 | −6.00 | −8.30 |

### Example 6. Protein Engineering and Assaying the ALDH Variant Library

TABLE 11
ALDH Michaelis-Menten Kinetic Parameters*

| | Homolog 3 (C-terminal tag) | | | |
|---|---|---|---|---|
| | HHA- | Adipyl- | Acetyl- | Succinyl- |
| | CoA | CoA | CoA | CoA |
| Vmax (uM/min | 8.0 | 9.3 | 33.5 | 27.8 |
| | (7.0-9.1) | (8.6-10.1) | (30.5-36.9) | (24.4-31.8) |
| Km (mM) | 2.4 | 1.1 | 2.1 | 1.7 |
| | (1.5-3.7) | (0.8-1.4) | (1.5-2.9) | (1.1-2.6) |
| kcat (s⁻¹) | 6.7 | 3.1 | 0.056 | 0.23 |

*95% confidence intervals based on profile likelihood indicated in parenthesis

An ALDH variant library comprising about 1500 proteins was produced based on the template ALDH (homolog 3).

### 6.1. Primary Screen

Aldehyde dehydrogenases were expressed from an IPTG inducible plasmid in E. coli (ΔadhE) and tested in a cell lysate assay. In order to evaluate the impact of each ALDH variant, a plasmid containing a gene encoding a variant was transformed into a strain of E. coli that also included introduced genes encoding HHA-CoA pathway enzymes: 1) a beta ketothiolase, 2) a hydroxyadipyl-CoA dehydrogenase (HBD) 3) a crotonase, 4) trans-enoyl CoA reductase (TER) 5) a 6ACA-aldehyde dehydrogenase (ALD), 6) an alcohol dehydrogenase and 7) an CoA transferase/ligase. Alternatively, the gene encoding a variant was integrated onto the chromosome of an E. coli strain already expressing the same HHA-CoA pathway enzymes. The resulting strains were evaluated for enzymatic activity as described using similar processes as described in Example 2.1.

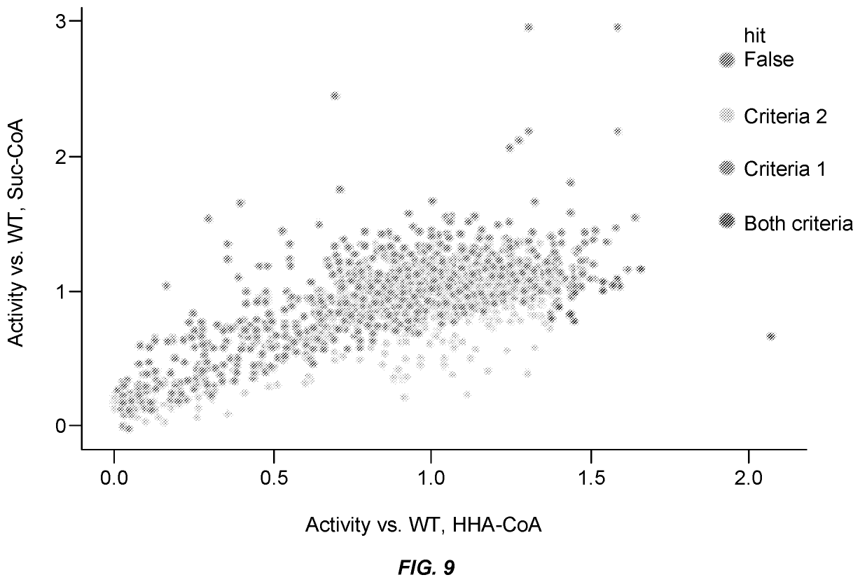

TABLE 12 shows that ALDH variants of homolog 6, nos. 393, 779, 872, 1046, 1047, 1230, 1232, 1234, 1265, 1280, 1304, 1339, and 1382 have the highest activity level and specificity level relative to homolog 3. Variants promoted top secondary screening are based on selection criteria: activity (1) or selectivity (2) and can be seen in FIG. 9.

TABLE 12
Exemplary Engineered ALDHs Using Homolog 3 (SEQ ID NO:
3) as the Template with Positional Alteration(s) and
Corresponding Activity on HHA-CoA and Succinyl-CoA

| | Mutations | Activity on | | Activity on | |
|---|---|---|---|---|---|
| | to | HHA-CoA | Active | succinyl-CoA | Active |
| Variant | homolog | relative to | HHA- | relative to | succinyl- |
| # | 3 | homolog 3 | CoA | homolog 3 | CoA |
| | WT | 1 | + | 1 | + |
| 1 | N154H | 0.98 | − | 1.02 | + |
| 2 | L307I | 1.124 | + | 0.928 | − |
| 3 | N288R | 0.944 | − | 1.015 | + |
| 4 | V321L | 1.064 | + | 1.038 | + |
| 5 | I251Y | 0.875 | − | 0.924 | − |
| 6 | I170L | 0.647 | − | 0.854 | − |
| 7 | D82E | 0.969 | − | 1.292 | + |
| 8 | L160V | 1.12 | + | 0.921 | − |
| 9 | Q3S | 1.067 | + | 0.954 | − |
| 10 | Q212A | 1.06 | + | 1.163 | + |
| 11 | R86V | 1.046 | + | 0.934 | − |
| 12 | K358N | 1.086 | + | 1.1 | + |
| 13 | T245M | 0.062 | − | 0.208 | − |
| 14 | N119Q | 0.62 | − | 0.33 | − |
| 15 | A382K | 1.04 | + | 0.984 | − |
| 16 | D352Q | 0.947 | − | 0.886 | − |
| 17 | E331S | 0.448 | − | 0.621 | − |
| 18 | L49Y | 0.192 | − | 0.491 | − |
| 19 | L333M | 1.058 | + | 0.946 | − |
| 20 | E276Q | 0.977 | − | 0.915 | − |
| 21 | V232I | 0.935 | − | 0.846 | − |
| 22 | V152L | 0.956 | − | 0.992 | − |
| 23 | N360S | 1.033 | + | 0.821 | − |
| 24 | I18K | 1.051 | + | 1.004 | + |
| 25 | I261L | 1.063 | + | 1.05 | + |
| 26 | D453E | 0.916 | − | 1.022 | + |
| 27 | V210H | 0.192 | − | 0.197 | − |
| 28 | E254Q | 0.96 | − | 1.069 | + |
| 29 | F430I | 0.697 | − | 1.324 | + |
| 30 | G289M | 0.815 | − | 1.235 | + |
| 31 | R325K | 0.78 | − | 1.336 | + |
| 32 | D64E | 0.626 | − | 0.462 | − |
| 33 | T30Q | 1.278 | + | 1.122 | + |
| 34 | Y43L | 1.018 | + | 1.065 | + |
| 35 | Q396R | 0.199 | − | 0.201 | − |
| 36 | C130T | 0.974 | − | 0.972 | − |
| 37 | P377D | 0.956 | − | 1.055 | + |
| 38 | S202N | 0.982 | − | 1.01 | + |
| 39 | K265Q | 1.267 | + | 1.099 | + |
| 40 | A53I | 0.647 | − | 1.484 | + |
| 41 | W457M | 0.026 | − | 0.21 | − |
| 42 | W78N | 1.052 | + | 1.131 | + |
| 43 | I366Y | 1.014 | + | 1.109 | + |
| 44 | Y26W | 1 | + | 1.009 | + |
| 45 | Q311R | 1.051 | + | 1.127 | + |
| 46 | V217F | 0.066 | − | 0.086 | − |
| 47 | V169L | 1.031 | + | 1.112 | + |
| 48 | Y432A | 0.108 | − | 0.315 | − |
| 49 | L196M | 0.97 | − | 1.152 | + |
| 50 | P194T | 1.181 | + | 1.092 | + |
| 51 | S178V | −0.027 | − | 0.273 | − |
| 52 | V215S | 1.319 | + | 1.427 | + |
| 53 | M80V | 1.094 | + | 0.925 | − |
| 54 | F286L | 0.963 | − | 1.082 | + |
| 55 | L338F | −0.092 | − | 0.248 | − |
| 56 | V217A | 1.022 | + | 1.005 | + |
| 57 | M175I | 1.153 | + | 1.009 | + |
| 58 | N115W | −0.097 | − | 0.197 | − |
| 59 | V76Y | −0.034 | − | 0.171 | − |
| 60 | T113F | −0.022 | − | 0.197 | − |
| 61 | I167V | 1.133 | + | 0.935 | − |
| 62 | A63Q | 1.319 | + | 1.113 | + |
| 63 | M121V | 1.177 | + | 1.108 | + |
| 64 | E94D | 1.277 | + | 1.091 | + |
| 65 | C246S | 1.273 | + | 1.36 | + |
| 66 | K256V | 1.324 | + | 1.113 | + |
| 67 | N424S | 0.259 | − | 0.306 | − |
| 68 | L9V | 1.147 | + | 0.933 | − |
| 69 | P93D | 1.347 | + | 0.983 | − |
| 70 | T171E | 1.258 | + | 1.064 | + |
| 71 | S275P | 1.054 | + | 0.972 | − |
| 72 | G209L | 0.309 | − | 0.188 | − |
| 73 | K40W | 1.072 | + | 0.923 | − |
| 74 | L435V | 1.454 | + | 1.295 | + |
| 75 | D224N | 1.135 | + | 1.113 | + |
| 76 | E259A | 1.152 | + | 1.047 | + |
| 77 | Y271F | 1.089 | + | 1.03 | + |
| 78 | K74R | 1.153 | + | 1.403 | + |
| 79 | W350V | 1.378 | + | 0.971 | − |
| 80 | P27D | 1.072 | + | 1.006 | + |
| 81 | Q3E | 0.98 | − | 0.948 | − |
| 82 | L48E | 1.074 | + | 0.884 | − |
| 83 | K234W | 1.379 | + | 0.827 | − |
| 84 | D239R | 0.007 | − | 0.153 | − |
| 85 | P291K | 1.137 | + | 1.544 | ++ |
| 86 | E2Q | 1.171 | + | 1.108 | + |
| 87 | K265R | 1.016 | + | 0.928 | − |
| 88 | P451W | 0.826 | − | 1.023 | + |
| 89 | W350E | 1.262 | + | 0.818 | − |
| 90 | F238R | 1.036 | + | 0.619 | − |
| 91 | I189S | 0.658 | − | 0.635 | − |
| 92 | V383M | 1.062 | + | 1.083 | + |
| 93 | V319F | 0.157 | − | 0.322 | − |
| 94 | V397I | 1.096 | + | 1.016 | + |
| 95 | D283E | 1.038 | + | 1.008 | + |
| 96 | A305I | 0.997 | − | 1.046 | + |
| 97 | A285L | 0.883 | − | 1.006 | + |
| 98 | M162I | 0.87 | − | 0.972 | − |
| 99 | Q227K | 1.113 | + | 1.09 | + |
| 100 | T346R | 0.948 | − | 0.954 | − |
| 101 | Q306H | 1.016 | + | 1.007 | + |
| 102 | H267Y | 0.97 | − | 1.011 | + |
| 103 | T346K | 0.978 | − | 1.017 | + |
| 104 | K280Q | 0.752 | − | 0.898 | − |
| 105 | K83Q | 0.963 | − | 0.848 | − |
| 106 | I135V | 0.959 | − | 0.946 | − |
| 107 | A386E | 0.86 | − | 0.979 | − |
| 108 | R444E | 0.739 | − | 0.754 | − |
| 109 | C323E | 0.978 | − | 0.968 | − |
| 110 | N240Q | 0.612 | − | 0.707 | − |
| 111 | M12I | 0.87 | − | 0.909 | − |
| 112 | F430Y | 0.029 | − | 0.206 | − |
| 113 | H355R | 0.925 | − | 0.904 | − |
| 114 | K72A | 0.915 | − | 0.203 | − |
| 115 | D7E | 1.025 | + | 0.931 | − |
| 116 | Q306K | 1.013 | + | 1.092 | + |
| 117 | R141Q | 0.08 | − | 0.188 | − |
| 118 | M175H | 1.154 | + | 1.38 | + |
| 119 | N154A | 0.976 | − | 0.949 | − |
| 120 | E276M | 0.943 | − | 1.007 | + |
| 121 | K11D | 1.043 | + | 0.96 | − |
| 122 | H355A | 1.182 | + | 0.987 | − |
| 123 | E99Q | 1.102 | + | 1.018 | + |
| 124 | I456F | 0.789 | − | 0.762 | − |
| 125 | M406Y | 0.958 | − | 0.621 | − |
| 126 | A158E | 1.165 | + | 0.953 | − |
| 127 | H355D | 1.141 | + | 0.968 | − |
| 128 | A184H | 0.984 | − | 0.988 | − |
| 129 | V100Y | 1.348 | + | 1.086 | + |
| 130 | I450M | 0.715 | − | 0.86 | − |
| 131 | E176Q | 0.984 | − | 1.144 | + |
| 132 | N132C | 0.694 | − | 0.791 | − |
| 133 | H373W | 1.004 | + | 1.005 | + |
| 134 | I118N | 0.164 | − | 1.031 | + |
| 135 | Y271K | 1.032 | + | 1.058 | + |
| 136 | Y201M | 0.841 | − | 0.903 | − |
| 137 | S427G | 0.951 | − | 0.783 | − |
| 138 | A382R | 0.727 | − | 0.91 | − |
| 139 | T346P | 1.22 | + | 0.848 | − |
| 140 | K280R | 0.71 | − | 0.888 | − |
| 141 | L157A | 1.057 | + | 0.829 | − |
| 142 | K90S | 1.136 | + | 0.901 | − |
| 143 | V35I | 0.656 | − | 0.796 | − |
| 144 | K335R | 1.041 | + | 0.871 | − |
| 145 | V232F | 0.983 | − | 0.741 | − |
| 146 | H267R | 1.133 | + | 1.084 | + |
| 147 | T149V | 1.105 | + | 1.161 | + |
| 148 | D316E | 1.142 | + | 1.055 | + |
| 149 | D55R | 1.161 | + | 1.295 | + |
| 150 | V31L | 1.239 | + | 1.19 | + |
| 151 | A111T | 1.376 | + | 0.818 | − |
| 152 | T25A | 1.096 | + | 1.029 | + |
| 153 | D407H | 1.251 | + | 1.057 | + |
| 154 | H379N | 1.139 | + | 1.006 | + |
| 155 | R282Q | 1.126 | + | 1.137 | + |
| 156 | K257A | 1.172 | + | 1.006 | + |
| 157 | S183Q | 1.24 | + | 1.013 | + |
| 158 | V152M | 1.009 | + | 1.018 | + |
| 159 | A179D | 0.574 | − | 0.537 | − |
| 160 | A382E | 0.771 | − | 0.89 | − |
| 161 | H434R | 0.094 | − | 0.182 | − |
| 162 | I92M | 0.863 | − | 0.929 | − |
| 163 | A299K | 1.21 | + | 1.359 | + |
| 164 | Q227R | 0.824 | − | 1.055 | + |
| 165 | E381M | 1.214 | + | 1.025 | + |
| 166 | V292I | 0.671 | − | 0.84 | − |
| 167 | Y270L | 1.092 | + | 0.981 | − |
| 168 | M436T | 0.742 | − | 0.932 | − |
| 169 | E455K | 1.057 | + | 1.001 | + |
| 170 | A142T | 0.319 | − | 0.421 | − |
| 171 | R91E | 1.126 | + | 0.91 | − |
| 172 | V445I | 0.701 | − | 0.927 | − |
| 173 | Y270F | 1.105 | + | 0.942 | − |
| 174 | K294S | 0.383 | − | 0.481 | − |
| 175 | K58R | 1.153 | + | 1.031 | + |
| 176 | S275G | 1.197 | + | 1.068 | + |
| 177 | K349S | 1.153 | + | 1.044 | + |
| 178 | K294N | 0.439 | − | 0.343 | − |
| 179 | T171P | 1.334 | + | 1.109 | + |
| 180 | A285V | 1.053 | + | 1.077 | + |
| 181 | V136L | 1.256 | + | 1.138 | + |
| 182 | I18E | 1.224 | + | 1.103 | + |
| 183 | E351D | 1.044 | + | 1.082 | + |
| 184 | R86K | 1.122 | + | 1.05 | + |
| 185 | P297A | 0.691 | − | 0.721 | − |
| 186 | V100L | 1.148 | + | 1.254 | + |
| 187 | K6F | 1.111 | + | 0.867 | − |
| 188 | L307V | 1.053 | + | 1.085 | + |
| 189 | S390T | 1.292 | + | 1.172 | + |
| 190 | I456L | 0.677 | − | 1.187 | + |
| 191 | A24S | 0.986 | − | 0.936 | − |
| 192 | A230R | 0.859 | − | 1.029 | + |
| 193 | S146T | 0.953 | − | 1.066 | + |
| 194 | S73A | 0.934 | − | 1.173 | + |
| 195 | D348E | 1.082 | + | 0.915 | − |
| 196 | I18T | 0.84 | − | 0.973 | − |
| 197 | L218I | 1.026 | + | 0.892 | − |
| 198 | A279E | 1.071 | + | 1.122 | + |
| 199 | D255E | 1.137 | + | 1.028 | + |
| 200 | K302A | 1.022 | + | 0.859 | − |
| 201 | E381W | 1.199 | + | 0.988 | − |
| 202 | V397W | 1.119 | + | 0.956 | − |
| 203 | A24R | 0.941 | − | 1.055 | + |
| 204 | D7Q | 1.103 | + | 1.155 | + |
| 205 | A405I | 0.465 | − | 0.508 | − |
| 206 | A34V | 1.095 | + | 1.047 | + |
| 207 | P253D | 0.843 | − | 0.865 | − |
| 208 | I89P | 0.261 | − | 0.348 | − |
| 209 | G312A | 1.062 | + | 1.058 | + |
| 210 | S174G | 0.911 | − | 0.988 | − |
| 211 | S300A | 1.055 | + | 1.111 | + |
| 212 | Y327W | 1.131 | + | 1.044 | + |
| 213 | M406K | 0.934 | − | 0.775 | − |
| 214 | M52L | 1.011 | + | 0.906 | − |
| 215 | A34L | 0.98 | − | 1.085 | + |
| 216 | E29K | 0.678 | − | 0.963 | − |
| 217 | S275A | 1.052 | + | 0.945 | − |
| 218 | H144L | 0.883 | − | 1.059 | + |
| 219 | T25N | 0.938 | − | 0.855 | − |
| 220 | V383L | 0.801 | − | 1.083 | + |
| 221 | L95K | 1.023 | + | 0.92 | − |
| 222 | S308A | 0.61 | − | 0.938 | − |
| 223 | K58Q | 0.948 | − | 0.762 | − |
| 224 | E455Q | 0.75 | − | 1.101 | + |
| 225 | V397A | 0.746 | − | 0.957 | − |
| 226 | T439Q | 0.491 | − | 0.431 | − |
| 227 | K11A | 0.817 | − | 1.114 | + |
| 228 | A34I | 0.978 | − | 0.932 | − |
| 229 | I380A | 0.849 | − | 0.852 | − |
| 230 | S145T | 1.032 | + | 1.225 | + |
| 231 | V321P | −0.013 | − | −0.032 | − |
| 232 | K205R | 0.872 | − | 0.988 | − |
| 233 | V445Y | 0.844 | − | 0.943 | − |
| 234 | Y271V | 0.971 | − | 1.086 | + |
| 235 | H373Y | 1.093 | + | 1.001 | + |
| 236 | I89L | 1.021 | + | 1.041 | + |
| 237 | T245I | 1.067 | + | 0.373 | − |
| 238 | Q21E | 0.993 | − | 0.906 | − |
| 239 | I304L | 1.101 | + | 1.164 | + |
| 240 | Y62I | 0.968 | − | 1.105 | + |
| 241 | V371M | 0.989 | − | 0.942 | − |
| 242 | G96N | 1.172 | + | 0.918 | − |
| 243 | V31I | 1.068 | + | 0.955 | − |
| 244 | V319L | 1.436 | + | 1.022 | + |
| 245 | S85A | 0.737 | − | 0.9 | − |
| 246 | I356M | 0.969 | − | 0.896 | − |
| 247 | N119I | 0.133 | − | 0.33 | − |
| 248 | F430L | 0.971 | − | 0.922 | − |
| 249 | N45Q | 0.773 | − | 1 | + |
| 250 | K266A | 0.997 | − | 0.825 | − |
| 251 | K329E | 0.685 | − | 0.939 | − |
| 252 | V136M | 0.869 | − | 0.827 | − |
| 253 | N61K | 0.643 | − | 0.994 | − |
| 254 | Y443H | 0.742 | − | 1.12 | + |
| 255 | I88L | 0.956 | − | 0.876 | − |
| 256 | M436L | 0.706 | − | 1.045 | + |
| 257 | V383Y | 0.836 | − | 0.659 | − |
| 258 | V232T | 0.953 | − | 0.772 | − |
| 259 | T324I | 1.074 | + | 1.093 | + |
| 260 | Y26L | 1.147 | + | 0.91 | − |
| 261 | Y443W | 1.202 | + | 1.045 | + |
| 262 | E151D | 1.324 | + | 0.954 | − |
| 263 | E295R | 1.078 | + | 1.138 | + |
| 264 | A24E | 1.067 | + | 0.847 | − |
| 265 | D44Q | 1.311 | + | 1.135 | + |
| 266 | K280T | 1.097 | + | 0.99 | − |
| 267 | V445F | 0.878 | − | 1.089 | + |
| 268 | I272V | 1.243 | + | 0.956 | − |
| 269 | K318R | 1.183 | + | 1.112 | + |
| 270 | E151K | 1.05 | + | 0.935 | − |
| 271 | V441L | 1.297 | + | 1.09 | + |
| 272 | L435F | 0.095 | − | 0.171 | − |
| 273 | Q168T | 1.253 | + | 1.039 | + |
| 274 | Y296W | 1.255 | + | 1.059 | + |
| 275 | L438V | 0.724 | − | 1.033 | + |
| 276 | A285I | 0.799 | − | 0.843 | − |
| 277 | V383F | 0.673 | − | 1.216 | + |
| 278 | Y296R | 0.334 | − | 0.363 | − |
| 279 | Y296V | 0.651 | − | 0.988 | − |
| 280 | A263E | 0.915 | − | 0.815 | − |
| 281 | E385Q | 0.733 | − | 1.186 | + |
| 282 | A14D | 0.635 | − | 0.659 | − |
| 283 | C323L | 0.717 | − | 1.118 | + |
| 284 | D82P | 0.887 | − | 0.872 | − |
| 285 | L435W | −0.036 | − | 0.156 | − |
| 286 | T245N | 0.202 | − | 0.279 | − |
| 287 | T439S | 1.05 | + | 0.953 | − |
| 288 | R79Y | 0.903 | − | 0.335 | − |
| 289 | Y271W | 1.219 | + | 1.028 | + |
| 290 | G96K | 0.97 | − | 0.987 | − |
| 291 | V98T | 1.209 | + | 1.038 | + |
| 292 | V319M | 1.403 | + | 1.207 | + |
| 293 | T359A | 1.195 | + | 0.976 | − |
| 294 | L438T | 1.122 | + | 1.003 | + |
| 295 | E259R | 1.069 | + | 1.01 | + |
| 296 | Y271R | 0.942 | − | 1.035 | + |
| 297 | D352H | 0.99 | − | 0.96 | − |
| 298 | V54L | 1.095 | + | 1.057 | + |
| 299 | P206V | 1.226 | + | 0.998 | − |
| 300 | M436F | 1.152 | + | 1.087 | + |
| 301 | Y296F | 0.945 | − | 0.889 | − |
| 302 | A393M | 0.775 | − | 0.93 | − |
| 303 | L438W | 0.926 | − | 0.866 | − |
| 304 | E362L | 0.7 | − | 1.255 | + |
| 305 | V210L | 0.131 | − | 0.167 | − |
| 306 | V185C | 0.762 | − | 0.964 | − |
| 307 | P253Q | 0.915 | − | 0.846 | − |
| 308 | P446E | 0.76 | − | 1.264 | + |
| 309 | C418F | 0.032 | − | 0.174 | − |
| 310 | L177V | 0.924 | − | 1.151 | + |
| 311 | K148Y | 0.875 | − | 0.873 | − |
| 312 | A53E | 0.51 | − | 0.717 | − |
| 313 | M134I | 0.879 | − | 0.833 | − |
| 314 | I450C | 0.924 | − | 0.791 | − |
| 315 | A159K | 1.084 | + | 1.105 | + |
| 316 | P112E | 0.04 | − | 0.228 | − |
| 317 | V228A | 1.048 | + | 0.914 | − |
| 318 | K40Q | 0.954 | − | 0.916 | − |
| 319 | I310G | 0.479 | − | 0.584 | − |
| 320 | R282K | 1.021 | + | 1.103 | + |
| 321 | E331D | 1.076 | + | 1.117 | + |
| 322 | A317G | 1.334 | + | 0.949 | − |
| 323 | M162A | 1.077 | + | 1.057 | + |
| 324 | K6R | 0.799 | − | 0.9 | − |
| 325 | V54T | 0.881 | − | 0.95 | − |
| 326 | V397P | 0.993 | − | 0.912 | − |
| 327 | A15K | 1.253 | + | 1.281 | + |
| 328 | L48M | 1.071 | + | 0.933 | − |
| 329 | I354L | 1.172 | + | 1.039 | + |
| 330 | A50G | 0.811 | − | 0.57 | − |
| 331 | T376N | 0.79 | − | 1.211 | + |
| 332 | Q311T | 0.907 | − | 0.86 | − |
| 333 | K329R | 1.054 | + | 0.873 | − |
| 334 | A159Q | 0.921 | − | 1.182 | + |
| 335 | Y62M | 0.958 | − | 1.02 | + |
| 336 | F23W | 0.53 | − | 1.435 | + |
| 337 | D32A | 0.656 | − | 0.508 | − |
| 338 | A279D | 0.974 | − | 1.169 | + |
| 339 | T37K | 1.011 | + | 0.945 | − |
| 340 | N375H | 1.054 | + | 0.913 | − |
| 341 | E172D | 1.065 | + | 0.983 | − |
| 342 | E155R | 1.109 | + | 1.028 | + |
| 343 | D316G | 1.078 | + | 1.051 | + |
| 344 | T359D | 0.987 | − | 0.891 | − |
| 345 | Q21A | 1.055 | + | 1.06 | + |
| 346 | L410F | 0.927 | − | 0.756 | − |
| 347 | S202K | 0.98 | − | 1.198 | + |
| 348 | P173G | 0.844 | − | 1.004 | + |
| 349 | G398S | 0.369 | − | 0.488 | − |
| 350 | V38F | 0.423 | − | 0.424 | − |
| 351 | K349G | 1.007 | + | 0.986 | − |
| 352 | K329P | 1.204 | + | 0.833 | − |
| 353 | L196I | 0.277 | − | 0.708 | − |
| 354 | Q396E | 1.075 | + | 0.66 | − |
| 355 | P253H | 1.098 | + | 0.956 | − |
| 356 | A14K | 0.432 | − | 0.562 | − |
| 357 | A386Q | 1.102 | + | 0.97 | − |
| 358 | I366K | 1.102 | + | 0.998 | − |
| 359 | Y449E | 0.862 | − | 0.905 | − |
| 360 | V232C | 0.99 | − | 0.918 | − |
| 361 | D186R | 0.867 | − | 0.909 | − |
| 362 | V370C | 1.156 | + | 1.065 | + |
| 363 | R325E | 1.08 | + | 0.946 | − |
| 364 | V169W | 0.872 | − | 0.861 | − |
| 365 | E99R | 1.091 | + | 0.899 | − |
| 366 | K205T | 0.632 | − | 0.683 | − |
| 367 | T452S | 1.038 | + | 0.974 | − |
| 368 | A334V | 1.265 | + | 1.028 | + |
| 369 | E22K | 1.034 | + | 0.925 | − |
| 370 | L166C | 1.127 | + | 1.076 | + |
| 371 | M260I | 0.927 | − | 0.856 | − |
| 372 | F344I | 1.13 | + | 1.136 | + |
| 373 | A262Q | 0.937 | − | 0.953 | − |
| 374 | H51A | 0.997 | − | 1.099 | + |
| 375 | R325D | 0.928 | − | 0.884 | − |
| 376 | L177T | 1.19 | + | 1.002 | + |
| 377 | A393I | 0.896 | − | 0.767 | − |
| 378 | E454A | 1.118 | + | 0.953 | − |
| 379 | Y62F | 1.077 | + | 1.111 | + |
| 380 | K303Q | 0.883 | − | 0.773 | − |
| 381 | K6Q | 0.563 | − | 0.61 | − |
| 382 | R141K | 0.777 | − | 0.609 | − |
| 383 | A382Q | 0.934 | − | 1.002 | + |
| 384 | G328D | 0.984 | − | 0.972 | − |
| 385 | I456V | 0.978 | − | 1.135 | + |
| 386 | R91D | 0.986 | − | 0.929 | − |
| 387 | G326D | 1.015 | + | 1.047 | + |
| 388 | T359S | 1.158 | + | 0.873 | − |
| 389 | I356L | 1.062 | + | 1.072 | + |
| 390 | N360T | 0.504 | − | 0.428 | − |
| 391 | P377I | 1.014 | + | 1.01 | + |
| 392 | L177A | 1.06 | + | 0.995 | − |
| 393 | S427T | 1.518 | ++ | 1.438 | + |
| 394 | I456W | 0.73 | − | 0.71 | − |
| 395 | S293A | 1.055 | + | 1.096 | + |
| 396 | V394I | 1.193 | + | 1.085 | + |
| 397 | D226E | 0.918 | − | 0.965 | − |
| 398 | K148L | 1.098 | + | 0.94 | − |
| 399 | P93E | 0.912 | − | 0.958 | − |
| 400 | D453P | 1.057 | + | 1.203 | + |
| 401 | Q3K | 1.106 | + | 1.141 | + |
| 402 | T376S | 0.903 | − | 0.982 | − |
| 403 | W350I | 1.041 | + | 0.959 | − |
| 404 | A229W | 0.501 | − | 0.642 | − |
| 405 | V292M | 0.63 | − | 0.907 | − |
| 406 | V232Q | 0.763 | − | 0.792 | − |
| 407 | S145C | 1.189 | + | 1.092 | + |
| 408 | A124V | 1.201 | + | 0.916 | − |
| 409 | G298A | 0.478 | − | 0.391 | − |
| 410 | Y433T | 1.022 | + | 0.95 | − |
| 411 | S146C | 0.914 | − | 0.821 | − |
| 412 | A278P | 0.895 | − | 1.124 | + |
| 413 | V38L | 1.184 | + | 0.942 | − |
| 414 | N119L | 0.364 | − | 0.725 | − |
| 415 | V70R | 1.122 | + | 1.025 | + |
| 416 | N154Q | 1.189 | + | 0.911 | − |
| 417 | I304G | 0.076 | − | 0.152 | − |
| 418 | A386K | 1.052 | + | 1.029 | + |
| 419 | W421Y | 0.531 | − | 0.481 | − |
| 420 | F344V | 1.103 | + | 1.191 | + |
| 421 | L281V | 1.067 | + | 1.199 | + |
| 422 | Y449R | 1.024 | + | 1.039 | + |
| 423 | L435Y | 0.004 | − | 0.156 | − |
| 424 | C126A | 1.076 | + | 1.25 | + |
| 425 | L361D | 0.458 | − | 0.443 | − |
| 426 | F23L | 0.906 | − | 1.086 | + |
| 427 | A458H | 1.012 | + | 0.978 | − |
| 428 | S425I | 1.128 | + | 1.121 | + |
| 429 | I380M | 0.871 | − | 0.775 | − |
| 430 | L402N | −0.002 | − | 0.208 | − |
| 431 | K143P | 0.637 | − | 0.568 | − |
| 432 | Y201Q | 0.256 | − | 0.706 | − |
| 433 | I372V | 1.183 | + | 1.077 | + |
| 434 | I261M | 1.01 | + | 1.159 | + |
| 435 | K90H | 1.079 | + | 1.054 | + |
| 436 | A53S | 0.886 | − | 1.075 | + |
| 437 | M406F | 1.237 | + | 0.916 | − |
| 438 | I304Y | 0.014 | − | 0.112 | − |
| 439 | W78E | 1.005 | + | 0.775 | − |
| 440 | I313L | 1.163 | + | 1.126 | + |
| 441 | M162F | 1.013 | + | 0.911 | − |
| 442 | R91Q | 1.104 | + | 0.999 | − |
| 443 | A111V | 0.775 | − | 0.484 | − |
| 444 | R17H | 0.932 | − | 0.951 | − |
| 445 | H144N | 0.901 | − | 0.976 | − |
| 446 | A101K | 0.517 | − | 0.496 | − |
| 447 | I372M | 1.017 | + | 1.115 | + |
| 448 | K6H | 0.786 | − | 0.864 | − |
| 449 | T25S | 0.988 | − | 1.034 | + |
| 450 | R236K | 0.605 | − | 0.622 | − |
| 451 | E428D | 1.312 | + | 1.293 | + |
| 452 | C69I | 0.756 | − | 0.788 | − |
| 453 | S322A | 1.023 | + | 1.052 | + |
| 454 | N411T | 1.245 | + | 1.501 | ++ |
| 455 | D283H | 1.021 | + | 1.125 | + |
| 456 | K302R | 0.975 | − | 1.053 | + |
| 457 | E364Q | 0.538 | − | 0.841 | − |
| 458 | Q122C | 0.816 | − | 0.625 | − |
| 459 | V150I | 1.043 | + | 1.085 | + |
| 460 | A285M | 0.863 | − | 0.849 | − |
| 461 | N408T | 0.712 | − | 1.173 | + |
| 462 | R282M | 1.064 | + | 1.546 | ++ |
| 463 | E254K | 1.021 | + | 1.003 | + |
| 464 | I313V | 1.044 | + | 1.057 | + |
| 465 | P332R | 1.019 | + | 1.279 | + |
| 466 | K148A | 1.05 | + | 0.981 | − |
| 467 | N154K | 1.077 | + | 1.119 | + |
| 468 | A230D | 1.019 | + | 0.942 | − |
| 469 | T431G | 0.824 | − | 0.952 | − |
| 470 | P332T | 1.074 | + | 1.203 | + |
| 471 | K90R | 1.107 | + | 1.073 | + |
| 472 | E2T | 1.118 | + | 1.157 | + |
| 473 | E385L | 0.861 | − | 0.997 | − |
| 474 | N45H | 1.005 | + | 1.057 | + |
| 475 | S293V | 0.968 | − | 1.074 | + |
| 476 | E331H | 0.179 | − | 0.383 | − |
| 477 | H379K | 0.98 | − | 1.039 | + |
| 478 | V38M | 0.994 | − | 0.991 | − |
| 479 | A184P | 0.627 | − | 0.563 | − |
| 480 | K58G | 0.954 | − | 0.918 | − |
| 481 | I251F | 1.019 | + | 1.047 | + |
| 482 | P27S | 0.942 | − | 1.047 | + |
| 483 | A269S | 0.924 | − | 0.958 | − |
| 484 | D330E | 1.011 | + | 0.994 | − |
| 485 | D220C | 0.958 | − | 0.975 | − |
| 486 | K256M | 1.047 | + | 1.072 | + |
| 487 | A262D | 0.928 | − | 1.039 | + |
| 488 | P194R | 1.131 | + | 1.176 | + |
| 489 | K6E | 0.961 | − | 0.975 | − |
| 490 | A279S | 1.008 | + | 1.07 | + |
| 491 | A223C | 1.077 | + | 1.069 | + |
| 492 | N288D | 0.972 | − | 0.924 | − |
| 493 | V445M | 0.818 | − | 1.073 | + |
| 494 | Y26T | 0.997 | − | 0.957 | − |
| 495 | L243V | 1.191 | + | 1.197 | + |
| 496 | M162Y | 1.115 | + | 1.02 | + |
| 497 | N437H | 0.024 | − | 0.174 | − |
| 498 | D283N | 1.084 | + | 1.118 | + |
| 499 | V321C | 1.061 | + | 1.043 | + |
| 500 | S390N | 0.073 | − | 0.151 | − |
| 501 | K148E | 1.013 | + | 0.846 | − |
| 502 | T30R | 0.947 | − | 0.969 | − |
| 503 | E276R | 1.045 | + | 1.036 | + |
| 504 | E247G | −0.044 | − | 0.088 | − |
| 505 | A159D | 1.008 | + | 1.001 | + |
| 506 | V219M | 1.128 | + | 1.144 | + |
| 507 | D44E | 0.757 | − | 0.854 | − |
| 508 | K294P | 0.492 | − | 0.595 | − |
| 509 | K318T | 0.996 | − | 1.042 | + |
| 510 | R79S | 0.939 | − | 0.727 | − |
| 511 | T114N | 0.722 | − | 0.507 | − |
| 512 | R282G | 1.02 | + | 1.171 | + |
| 513 | R36H | 0.945 | − | 0.948 | − |
| 514 | V169C | 1.121 | + | 1.079 | + |
| 515 | R282E | 0.664 | − | 0.907 | − |
| 516 | S390C | 1.27 | + | 1.342 | + |
| 517 | L338V | 0.412 | − | 0.234 | − |
| 518 | I135M | 0.943 | − | 1.119 | + |
| 519 | A34M | 0.985 | − | 1.093 | + |
| 520 | A229V | 1.102 | + | 1.185 | + |
| 521 | A101Y | 1.115 | + | 0.848 | − |
| 522 | I426V | 1.024 | + | 1.013 | + |
| 523 | P291L | 1.01 | + | 0.952 | − |
| 524 | R391M | 0.034 | − | 0.142 | − |
| 525 | Y347F | 1.031 | + | 0.932 | − |
| 526 | V120T | 1.398 | + | 0.913 | − |
| 527 | N45R | 1.251 | + | 1.059 | + |
| 528 | E331M | 0.022 | − | 0.144 | − |
| 529 | D222T | 1.274 | + | 1.069 | + |
| 530 | A269G | 0.925 | − | 0.914 | − |
| 531 | K234Q | 1.348 | + | 0.92 | − |
| 532 | L243M | 0.456 | − | 1.284 | + |
| 533 | L438C | 1.259 | + | 1.163 | + |
| 534 | Q311K | 0.915 | − | 0.974 | − |
| 535 | Y449F | 0.785 | − | 1.194 | + |
| 536 | S425G | 1.035 | + | 1.315 | + |
| 537 | I354V | 0.968 | − | 0.998 | − |
| 538 | T25K | 0.863 | − | 1.234 | + |
| 539 | K303W | 0.819 | − | 0.808 | − |
| 540 | A357C | 0.873 | − | 1.404 | + |
| 541 | D220F | 0.491 | − | 0.603 | − |
| 542 | E29Q | 0.967 | − | 1.285 | + |
| 543 | I104V | 0.885 | − | 0.947 | − |
| 544 | H267N | 0.863 | − | 1.01 | + |
| 545 | K265A | 0.598 | − | 0.803 | − |
| 546 | V319I | 0.946 | − | 0.997 | − |
| 547 | V219F | 0.892 | − | 1.379 | + |
| 548 | E94N | 0.906 | − | 0.982 | − |
| 549 | A230P | 1.184 | + | 1.021 | + |
| 550 | L166A | 0.775 | − | 0.883 | − |
| 551 | V147I | 1.118 | + | 1.052 | + |
| 552 | P253V | 0.851 | − | 0.979 | − |
| 553 | H379V | 1.307 | + | 1.185 | + |
| 554 | K302D | 0.906 | − | 0.881 | − |
| 555 | L95R | 1.221 | + | 1.108 | + |
| 556 | G87T | 0.909 | − | 0.939 | − |
| 557 | A384S | 1.349 | + | 1.088 | + |
| 558 | V371I | 0.875 | − | 1.125 | + |
| 559 | H373F | 1.116 | + | 0.942 | − |
| 560 | T452D | 0.993 | − | 0.988 | − |
| 561 | Q227M | 1.131 | + | 0.97 | − |
| 562 | R141Y | 0.253 | − | 0.263 | − |
| 563 | N375Q | 1.133 | + | 1.065 | + |
| 564 | I88V | 0.976 | − | 0.995 | − |
| 565 | K318Y | 0.851 | − | 1.255 | + |
| 566 | L97M | 1.064 | + | 1.125 | + |
| 567 | Y62A | 0.866 | − | 1.425 | + |
| 568 | T414M | −0.013 | − | 0.184 | − |
| 569 | K6P | 0.273 | − | 0.453 | − |
| 570 | V394K | −0.064 | − | 0.143 | − |
| 571 | Q248D | 0.545 | − | 0.986 | − |
| 572 | P314A | 0.939 | − | 0.974 | − |
| 573 | I233V | 0.829 | − | 1.288 | + |
| 574 | I450F | 0.851 | − | 1.028 | + |
| 575 | E295K | 0.881 | − | 1.199 | + |
| 576 | E22A | 1.007 | + | 1.07 | + |
| 577 | A230Q | 0.973 | − | 0.997 | − |
| 578 | G96H | 0.765 | − | 0.998 | − |
| 579 | D44R | 0.972 | − | 1.079 | + |
| 580 | L264M | 0.968 | − | 0.943 | − |
| 581 | V38W | −0.079 | − | 0.081 | − |
| 582 | K257F | 0.958 | − | 0.916 | − |
| 583 | E22Q | 1.051 | + | 0.989 | − |
| 584 | N154R | 0.936 | − | 1.036 | + |
| 585 | L166I | 1.05 | + | 1.078 | + |
| 586 | Q248T | 0.445 | − | 0.554 | − |
| 587 | C323I | 1.085 | + | 0.906 | − |
| 588 | Y432P | 0.03 | − | 0.189 | − |
| 589 | M182L | 0.955 | − | 0.872 | − |
| 590 | P253E | 0.723 | − | 0.746 | − |
| 591 | K148H | 0.95 | − | 1.029 | + |
| 592 | M363Y | 0.943 | − | 1.468 | + |
| 593 | I272M | 1.011 | + | 1.243 | + |
| 594 | E94A | 0.85 | − | 1.287 | + |
| 595 | Q21P | 0.716 | − | 0.882 | − |
| 596 | G180H | 0.539 | − | 0.853 | − |
| 597 | E295A | 0.882 | − | 1.133 | + |
| 598 | S183K | 1.047 | + | 1.54 | ++ |
| 599 | M162L | 1.045 | + | 1.093 | + |
| 600 | H267A | 0.736 | − | 1.203 | + |
| 601 | L48D | 1.048 | + | 0.98 | − |
| 602 | C323G | 0.577 | − | 0.933 | − |
| 603 | A343C | 1.26 | + | 1.144 | + |
| 604 | Q20M | 0.881 | − | 1.284 | + |
| 605 | A384G | 1.287 | + | 1.062 | + |
| 606 | E364Y | 0.225 | − | 0.641 | − |
| 607 | I88M | 1.363 | + | 1.198 | + |
| 608 | L218V | 1.019 | + | 0.961 | − |
| 609 | R17Q | 0.824 | − | 0.838 | − |
| 610 | G328T | 0.894 | − | 1.008 | + |
| 611 | V219L | 1.18 | + | 1.145 | + |
| 612 | E378K | 1.088 | + | 1.027 | + |
| 613 | I456M | 0.998 | − | 1.116 | + |
| 614 | K358R | 1.093 | + | 1.025 | + |
| 615 | N411G | 1.022 | + | 0.687 | − |
| 616 | Q21K | 0.97 | − | 1.072 | + |
| 617 | A299Q | 1.211 | + | 1.482 | + |
| 618 | D32E | 1.055 | + | 1.208 | + |
| 619 | K58E | 0.93 | − | 0.746 | − |
| 620 | A262E | 0.952 | − | 0.963 | − |
| 621 | F344L | 1.001 | + | 1.438 | + |
| 622 | Y26F | 0.773 | − | 1.192 | + |
| 623 | N411P | 1.333 | + | 1.001 | + |
| 624 | Y296H | 0.929 | − | 1.566 | ++ |
| 625 | A305V | 1.292 | + | 1.358 | + |
| 626 | S85T | 0.836 | − | 1.074 | + |
| 627 | D222S | 1.274 | + | 1.122 | + |
| 628 | T37R | 1.387 | + | 1.339 | + |
| 629 | V445L | 0.932 | − | 1.153 | + |
| 630 | V150M | 1.081 | + | 1.119 | + |
| 631 | A278E | 1.25 | + | 1.252 | + |
| 632 | M436I | 1.277 | + | 1.174 | + |
| 633 | I450V | 1.329 | + | 1.15 | + |
| 634 | S293I | 1.12 | + | 1.128 | + |
| 635 | Y270I | 1.074 | + | 1.097 | + |
| 636 | H434Q | 0.207 | − | 0.249 | − |
| 637 | K198Q | 0.963 | − | 0.726 | − |
| 638 | V38I | 1.167 | + | 1.168 | + |
| 639 | Y271H | 1.025 | + | 1.167 | + |
| 640 | W78H | 1.326 | + | 1.093 | + |
| 641 | V383I | 1.053 | + | 1.054 | + |
| 642 | A156I | 1.214 | + | 1.16 | + |
| 643 | I104Y | 0.857 | − | 0.903 | − |
| 644 | Y449H | 1.276 | + | 1.109 | + |
| 645 | A63D | 1.222 | + | 1.047 | + |
| 646 | S300R | 0.981 | − | 0.925 | − |
| 647 | K102E | 0.92 | − | 0.616 | − |
| 648 | T245S | 0.156 | − | 0.633 | − |
| 649 | T30K | 1.028 | + | 1.087 | + |
| 650 | Q248E | 0.865 | − | 1.099 | + |
| 651 | E381K | 1.096 | + | 1.06 | + |
| 652 | A230K | 1.216 | + | 1.203 | + |
| 653 | V54M | 0.731 | − | 0.931 | − |
| 654 | S133T | 1.208 | + | 1.103 | + |
| 655 | E381R | 1.037 | + | 1.059 | + |
| 656 | Q396S | 1.245 | + | 1.172 | + |
| 657 | A269C | 0.939 | − | 0.933 | − |
| 658 | Q3M | 0.355 | − | 0.618 | − |
| 659 | S293N | 1.209 | + | 1.247 | + |
| 660 | W350L | 1.136 | + | 1.036 | + |
| 661 | P173D | 0.11 | − | 0.462 | − |
| 662 | T439R | 0 | − | 0.127 | − |
| 663 | G290H | 1.094 | + | 1.064 | + |
| 664 | V250I | 0.829 | − | 0.916 | − |
| 665 | N165H | 1.171 | + | 1.054 | + |
| 666 | T113S | 0.908 | − | 0.741 | − |
| 667 | G398C | 0.681 | − | 0.831 | − |
| 668 | L416M | 0.557 | − | 0.737 | − |
| 669 | V106I | 0.785 | − | 0.933 | − |
| 670 | M59I | 0.428 | − | 0.716 | − |
| 671 | T431S | 0.914 | − | 1.04 | + |
| 672 | I189F | −0.029 | − | 0.205 | − |
| 673 | L49I | 1.233 | + | 1.045 | + |
| 674 | V150G | 0.899 | − | 1.038 | + |
| 675 | R444P | 0.841 | − | 1.014 | + |
| 676 | A200C | 1.051 | + | 1.059 | + |
| 677 | Q3R | 0.898 | − | 0.986 | − |
| 678 | I107C | 1.122 | + | 1.046 | + |
| 679 | A24K | 1.052 | + | 1.142 | + |
| 680 | S275E | 1.045 | + | 1.142 | + |
| 681 | E276D | 1.129 | + | 1.142 | + |
| 682 | T324V | 1.043 | + | 1.169 | + |
| 683 | L438Y | 1.184 | + | 1.162 | + |
| 684 | G273C | 1.155 | + | 1.176 | + |
| 685 | L281M | 1.08 | + | 1.23 | + |
| 686 | S442G | 0.734 | − | 0.857 | − |
| 687 | G96R | 0.988 | − | 1.204 | + |
| 688 | N437Q | 1.164 | + | 1.195 | + |
| 689 | Y433R | 1.072 | + | 1.221 | + |
| 690 | N408S | 0.743 | − | 1.201 | + |
| 691 | N437Y | 0.017 | − | 0.196 | − |
| 692 | I310V | 1.132 | + | 1.089 | + |
| 693 | V397F | 0.937 | − | 1.038 | + |
| 694 | S442A | 0.957 | − | 0.92 | − |
| 695 | W78F | 1.009 | + | 1.004 | + |
| 696 | I118A | 0.297 | − | 1.527 | ++ |
| 697 | I251L | 1.077 | + | 1.049 | + |
| 698 | K294E | 0.079 | − | 0.224 | − |
| 699 | L218F | 0.977 | − | 1.11 | + |
| 700 | R141E | −0.066 | − | 0.142 | − |
| 701 | H368L | 0.041 | − | 0.21 | − |
| 702 | T30D | 1.119 | + | 1.066 | + |
| 703 | L225V | 1.137 | + | 1.042 | + |
| 704 | S308T | 1.088 | + | 1.018 | + |
| 705 | D453H | 1.017 | + | 1.187 | + |
| 706 | Q311S | 1.14 | + | 1.121 | + |
| 707 | I366F | 0.879 | − | 0.987 | − |
| 708 | A458G | 1.11 | + | 1.143 | + |
| 709 | E172G | 1.017 | + | 1.164 | + |
| 710 | A184S | 1.15 | + | 1.136 | + |
| 711 | L402A | 0.268 | − | 0.445 | − |
| 712 | A262K | 1.043 | + | 1.068 | + |
| 713 | P446K | 1.238 | + | 1.082 | + |
| 714 | L338I | 0.262 | − | 0.409 | − |
| 715 | A14Q | 0.997 | − | 0.872 | − |
| 716 | K102Q | 1.085 | + | 0.921 | − |
| 717 | K329D | 1.202 | + | 0.911 | − |
| 718 | P377V | 1.057 | + | 1.053 | + |
| 719 | M448W | 1.008 | + | 1.064 | + |
| 720 | D32N | 1.221 | + | 1.104 | + |
| 721 | Y8D | 0.807 | − | 0.799 | − |
| 722 | M134C | 0.965 | − | 0.982 | − |
| 723 | E459D | 1.086 | + | 1.006 | + |
| 724 | K90E | 1.217 | + | 1.066 | + |
| 725 | I189T | 0.901 | − | 0.905 | − |
| 726 | D407Q | 1.236 | + | 1.168 | + |
| 727 | L243T | 0.569 | − | 1.056 | + |
| 728 | V371A | 1.072 | + | 1.126 | + |
| 729 | L49M | 0.809 | − | 1.019 | + |
| 730 | R36E | 0.611 | − | 0.662 | − |
| 731 | D316N | 1.127 | + | 1.1 | + |
| 732 | Y432F | 0.947 | − | 1.045 | + |
| 733 | I170V | 1.102 | + | 1.208 | + |
| 734 | P297T | 0.562 | − | 0.768 | − |
| 735 | Y443R | 1.108 | + | 1.147 | + |
| 736 | V250L | 0.958 | − | 1.093 | + |
| 737 | D239H | 0.049 | − | 0.165 | − |
| 738 | A343T | 0.9 | − | 1.054 | + |
| 739 | S183D | 0.903 | − | 0.742 | − |
| 740 | N274E | 1.142 | + | 1.035 | + |
| 741 | Q216H | 0.666 | − | 0.598 | − |
| 742 | P388R | 1.079 | + | 0.998 | − |
| 743 | E258D | 1.299 | + | 1.091 | + |
| 744 | K84Q | 1.157 | + | 0.958 | − |
| 745 | I450L | 0.815 | − | 0.79 | − |
| 746 | L416F | 0.02 | − | 0.126 | − |
| 747 | A230E | 1.172 | + | 0.962 | − |
| 748 | I320Y | 1.047 | + | 1.084 | + |
| 749 | K33R | 1.24 | + | 1.074 | + |
| 750 | V301I | 0.942 | − | 0.91 | − |
| 751 | K33A | 1.297 | + | 1.036 | + |
| 752 | I251V | 0.948 | − | 0.916 | − |
| 753 | T400I | 1.091 | + | 1.135 | + |
| 754 | D239Q | 0.08 | − | 0.372 | − |
| 755 | E295Q | 1.177 | + | 1.305 | + |
| 756 | H267Q | 0.916 | − | 1.127 | + |
| 757 | I310L | 1.067 | + | 1.13 | + |
| 758 | E247P | 0.039 | − | 0.173 | − |
| 759 | V13L | 0.937 | − | 1.262 | + |
| 760 | E259K | 0.958 | − | 1.161 | + |
| 761 | D277Q | 1.147 | + | 1.116 | + |
| 762 | N165G | 0.899 | − | 1.05 | + |
| 763 | S146A | 0.606 | − | 0.914 | − |
| 764 | S183A | 1.032 | + | 1.051 | + |
| 765 | V70L | 0.804 | − | 0.95 | − |
| 766 | A179N | 0.95 | − | 1.072 | + |
| 767 | P297Q | 0.459 | − | 0.624 | − |
| 768 | C109I | 0.448 | − | 0.942 | − |
| 769 | A334S | 1.226 | + | 0.981 | − |
| 770 | I66L | 0.735 | − | 0.828 | − |
| 771 | K65R | 1.174 | + | 1.133 | + |
| 772 | V117N | 0.691 | − | 1.111 | + |
| 773 | W457Y | 0.476 | − | 0.832 | − |
| 774 | I208V | 1.145 | + | 0.957 | − |
| 775 | G399A | 0.877 | − | 1.38 | + |
| 776 | N71E | 1.003 | + | 0.467 | − |
| 777 | A207V | 1.183 | + | 0.973 | − |
| 778 | M80L | 0.812 | − | 1.036 | + |
| 779 | C246A | 1.511 | ++ | 1.373 | + |
| 780 | M182H | 0.391 | − | 0.491 | − |
| 781 | N71H | 1.315 | + | 0.935 | − |
| 782 | I387M | 1.148 | + | 0.944 | − |
| 783 | K234F | 1.379 | + | 0.788 | − |
| 784 | Q122F | 0.895 | − | 0.411 | − |
| 785 | E254R | 1.076 | + | 1.012 | + |
| 786 | E362T | 1.228 | + | 1.147 | + |
| 787 | L196V | 1.089 | + | 0.969 | − |
| 788 | V120A | 1.25 | + | 0.764 | − |
| 789 | A142S | 0.956 | − | 0.78 | − |
| 790 | G211L | −0.099 | − | 0.05 | − |
| 791 | K81A | 1.143 | + | 1.155 | + |
| 792 | N123K | 0.181 | − | 0.237 | − |
| 793 | K74D | 0.817 | − | 0.676 | − |
| 794 | K234A | 1.179 | + | 0.79 | − |
| 795 | N423K | 0.778 | − | 0.77 | − |
| 796 | T414F | 0.133 | − | 0.163 | − |
| 797 | L333V | 0.989 | − | 0.893 | − |
| 798 | Y432W | 0.919 | − | 1.012 | + |
| 799 | N214S | 1.021 | + | 1.204 | + |
| 800 | V321I | 1.04 | + | 0.954 | − |
| 801 | L342M | 1.049 | + | 1.308 | + |
| 802 | F345Y | 1.07 | + | 1.051 | + |
| 803 | T30E | 0.317 | − | 0.505 | − |
| 804 | I104F | 1.126 | + | 1.058 | + |
| 805 | S16A | 1.038 | + | 1.304 | + |
| 806 | T359N | 1.095 | + | 0.978 | − |
| 807 | L187H | 0.855 | − | 1.124 | + |
| 808 | M260V | 1.039 | + | 0.894 | − |
| 809 | C69I | 0.92 | − | 1.012 | + |
| 810 | V215N | 1.304 | + | 1.359 | + |
| 811 | N401G | 1.318 | + | 0.941 | − |
| 812 | V35C | 1.134 | + | 1.116 | + |
| 813 | I233L | 1.277 | + | 1.024 | + |
| 814 | T245V | 0.612 | − | 0.362 | − |
| 815 | G60A | 0.027 | − | 0.213 | − |
| 816 | N411A | 1.389 | + | 0.885 | − |
| 817 | V110M | 0.012 | − | 0.256 | − |
| 818 | Y347V | 1.277 | + | 0.959 | − |
| 819 | I42C | 1.376 | + | 1.217 | + |
| 820 | P206R | 0.124 | − | 0.176 | − |
| 821 | S203N | 1.285 | + | 0.997 | − |
| 822 | V100M | 1.488 | + | 1.216 | + |
| 823 | I118L | 0.429 | − | 0.734 | − |
| 824 | F344M | 1.09 | + | 0.971 | − |
| 825 | S178A | 0.437 | − | 0.581 | − |
| 826 | V110L | 0.134 | − | 0.394 | − |
| 827 | A41V | 1.108 | + | 1.055 | + |
| 828 | G241N | 0.066 | − | 0.162 | − |
| 829 | T113W | 0.228 | − | 0.187 | − |
| 830 | Q248K | 0.1 | − | 0.024 | − |
| 831 | G398P | 0.042 | − | 0.156 | − |
| 832 | N288E | 1.198 | + | 0.899 | − |
| 833 | V35L | 0.747 | − | 0.809 | − |
| 834 | K329N | 1.102 | + | 0.972 | − |
| 835 | R36K | 1.161 | + | 1.09 | + |
| 836 | N401H | 1.31 | + | 1.02 | + |
| 837 | A207S | 0.946 | − | 0.679 | − |
| 838 | P206K | 0.127 | − | 0.116 | − |
| 839 | G403S | 0.223 | − | 0.462 | − |
| 840 | R79W | 1.239 | + | 0.549 | − |
| 841 | L218Y | 0.718 | − | 0.864 | − |
| 842 | A207C | 1.463 | + | 1.071 | + |
| 843 | L402F | 0.833 | − | 0.974 | − |
| 844 | I189M | 0.452 | − | 0.911 | − |
| 845 | Y433K | 1.185 | + | 1.144 | + |
| 846 | R79D | 1.178 | + | 0.398 | − |
| 847 | G312D | 1.233 | + | 1.066 | + |
| 848 | N61R | 1.113 | + | 1.064 | + |
| 849 | G193S | 0.313 | − | 0.57 | − |
| 850 | P194K | 1.334 | + | 1.25 | + |
| 851 | R236C | 0.027 | − | 0.319 | − |
| 852 | G209E | 0.063 | − | 0.143 | − |
| 853 | H355Q | 1.21 | + | 1.021 | + |
| 854 | S183H | 1.301 | + | 1.108 | + |
| 855 | H434D | 0.112 | − | 0.14 | − |
| 856 | P194A | 1.259 | + | 1.025 | + |
| 857 | F23Y | 1.178 | + | 1.199 | + |
| 858 | E454D | 1.238 | + | 1.095 | + |
| 859 | F345V | 1.002 | + | 1.053 | + |
| 860 | V341I | 1.122 | + | 1.033 | + |
| 861 | S146N | 1.039 | + | 0.801 | − |
| 862 | W78R | 0.903 | − | 1.011 | + |
| 863 | G131R | 0.867 | − | 1.07 | + |
| 864 | N61E | 1.045 | + | 0.838 | − |
| 865 | I107V | 1.209 | + | 1.095 | + |
| 866 | Q248S | −0.009 | − | 0.064 | − |
| 867 | G195A | 0.653 | − | 0.916 | − |
| 868 | N214K | 0.054 | − | 0.076 | − |
| 869 | F286I | 1.098 | + | 1.22 | + |
| 870 | E428H | 0.086 | − | 0.17 | − |
| 871 | S369T | −0.062 | − | 0.125 | − |
| 872 | T149I | 1.555 | ++ | 1.354 | + |
| 873 | S137R | 0.611 | − | 0.485 | − |
| 874 | E315D | 1.084 | + | 1.002 | + |
| 875 | Q122R | 0.021 | − | 0.124 | − |
| 876 | R236W | 0.063 | − | 0.119 | − |
| 877 | V54I | 0.84 | − | 0.887 | − |
| 878 | M182I | 0.76 | − | 0.659 | − |
| 879 | H139S | 0.112 | − | 0.575 | − |
| 880 | M59Y | 0.644 | − | 0.795 | − |
| 881 | K198E | 0.539 | − | 0.51 | − |
| 882 | E94G | 1.166 | + | 1.088 | + |
| 883 | A200I | 0.482 | − | 0.715 | − |
| 884 | F286W | 0.847 | − | 0.87 | − |
| 885 | M59F | 0.429 | − | 0.562 | − |
| 886 | G235A | 0.08 | − | 0.263 | − |
| 887 | A393W | −0.059 | − | 0.169 | − |
| 888 | N115R | −0.064 | − | 0.186 | − |
| 889 | P297V | 0.81 | − | 1.021 | + |
| 890 | M125I | 1.246 | + | 1.361 | + |
| 891 | L342V | 0.998 | − | 0.949 | − |
| 892 | M121A | 0.739 | − | 1.315 | + |
| 893 | V217I | 0.86 | − | 0.843 | − |
| 894 | W350F | 1.156 | + | 0.973 | − |
| 895 | I42L | 1.154 | + | 1.08 | + |
| 896 | W78Y | 1.185 | + | 1.039 | + |
| 897 | N401D | 1.193 | + | 0.719 | − |
| 898 | T149L | 1.347 | + | 1.173 | + |
| 899 | Y43R | 0.931 | − | 0.914 | − |
| 900 | I189V | 1.17 | + | 0.853 | − |
| 901 | S369A | 0.66 | − | 0.595 | − |
| 902 | S203H | 1.249 | + | 1.04 | + |
| 903 | I153F | 1.041 | + | 0.988 | − |
| 904 | L187K | 1.032 | + | 1.098 | + |
| 905 | S174D | 0.92 | − | 0.862 | − |
| 906 | I233M | 0.978 | − | 0.96 | − |
| 907 | P297L | 0.792 | − | 0.8 | − |
| 908 | C130S | 1.143 | + | 1.195 | + |
| 909 | L281I | 1.098 | + | 1.179 | + |
| 910 | F238M | 1.458 | + | 1.274 | + |
| 911 | K65E | −0.037 | − | 0.26 | − |
| 912 | T400S | 1.101 | + | 0.85 | − |
| 913 | M59L | 0.467 | − | 0.525 | − |
| 914 | C188I | 0.041 | − | 0.332 | − |
| 915 | E151R | 0.987 | − | 1.002 | + |
| 916 | Y62V | 1.031 | + | 1.336 | + |
| 917 | K81E | 0.687 | − | 0.657 | − |
| 918 | R86E | 0.154 | − | 0.301 | − |
| 919 | M406L | 1.06 | + | 1.506 | ++ |
| 920 | V215A | 1.46 | + | 1.399 | + |
| 921 | N274D | 1.046 | + | 1.003 | + |
| 922 | K81R | 1.241 | + | 1.165 | + |
| 923 | N119M | 0.296 | − | 0.235 | − |
| 924 | T400F | 1.248 | + | 2.053 | ++ |
| 925 | E385R | 1.052 | + | 1.009 | + |
| 926 | K335Q | 0.664 | − | 0.732 | − |
| 927 | C69N | 0.935 | − | 1.042 | + |
| 928 | M125L | 1.024 | + | 1.406 | + |
| 929 | F430V | 0.912 | − | 1.344 | + |
| 930 | V70I | 0.997 | − | 0.977 | − |
| 931 | I92D | 1.343 | + | 1.031 | + |
| 932 | P388H | 1.1 | + | 1.144 | + |
| 933 | K164D | 1.33 | + | 1.033 | + |
| 934 | I366A | 1.367 | + | 0.996 | − |
| 935 | C252V | 1.081 | + | 0.987 | − |
| 936 | I366L | 1.25 | + | 1.146 | + |
| 937 | N71F | 1.116 | + | 0.762 | − |
| 938 | T376D | 1.278 | + | 1.113 | + |
| 939 | V76F | −0.005 | − | 0.154 | − |
| 940 | H139G | 0.22 | − | 0.627 | − |
| 941 | L128I | 1.232 | + | 1.104 | + |
| 942 | Q122Y | 0.1 | − | 0.122 | − |
| 943 | L410I | 0.929 | − | 1.097 | + |
| 944 | I104L | 0.831 | − | 1.069 | + |
| 945 | T171N | 1.336 | + | 1.166 | + |
| 946 | I208T | 0.441 | − | 0.438 | − |
| 947 | A111Y | 0.212 | − | 0.226 | − |
| 948 | Q122H | 1.452 | + | 0.767 | − |
| 949 | L181I | 1.198 | + | 1.075 | + |
| 950 | R391T | −0.002 | − | 0.145 | − |
| 951 | V120P | 0.492 | − | 0.576 | − |
| 952 | S73T | 1.004 | + | 1.657 | ++ |
| 953 | L243C | −0.035 | − | 0.233 | − |
| 954 | F286Y | 1.06 | + | 0.877 | − |
| 955 | T400V | 0.932 | − | 1 | + |
| 956 | Y433H | 1.357 | + | 1.093 | + |
| 957 | K266E | 1.099 | + | 0.954 | − |
| 958 | I104M | 1.101 | + | 1.156 | + |
| 959 | S202A | 1.189 | + | 1.05 | + |
| 960 | A124L | 1.385 | + | 1.052 | + |
| 961 | M121L | 1.464 | + | 1.042 | + |
| 962 | C109A | 0.423 | − | 0.649 | − |
| 963 | I118M | 0.393 | − | 1.092 | + |
| 964 | N71Y | 1.14 | + | 0.559 | − |
| 965 | A405F | 0.836 | − | 1.119 | + |
| 966 | A67E | 1.395 | + | 0.894 | − |
| 967 | V250M | 1.262 | + | 0.945 | − |
| 968 | V197I | 1.003 | + | 0.883 | − |
| 969 | R236A | 0.687 | − | 0.916 | − |
| 970 | K256I | 1.055 | + | 1.009 | + |
| 971 | T431N | 1.151 | + | 1.012 | + |
| 972 | F345M | 1.074 | + | 1.049 | + |
| 973 | Q212P | 1.307 | + | 1.36 | + |
| 974 | A111I | 0.923 | − | 0.354 | − |
| 975 | N423R | 1.135 | + | 1.313 | + |
| 976 | W77Y | 1.028 | + | 1.201 | + |
| 977 | E364F | 0.128 | − | 0.395 | − |
| 978 | A127M | 0.969 | − | 0.966 | − |
| 979 | C69M | 0.904 | − | 1.255 | + |
| 980 | N240A | 0.545 | − | 0.573 | − |
| 981 | G209M | 0.144 | − | 0.297 | − |
| 982 | V197M | 0.737 | − | 0.563 | − |
| 983 | K148R | 1.254 | + | 0.999 | − |
| 984 | L49W | 0.325 | − | 0.739 | − |
| 985 | K129A | 1.081 | + | 0.915 | − |
| 986 | I387V | 1.147 | + | 1.057 | + |
| 987 | K102R | 1.254 | + | 0.889 | − |
| 988 | K33Q | 1.008 | + | 1.024 | + |
| 989 | T245A | 0.104 | − | 0.38 | − |
| 990 | L157I | 1.044 | + | 1.035 | + |
| 991 | M12A | 1.14 | + | 1.067 | + |
| 992 | A200V | 0.488 | − | 0.821 | − |
| 993 | P297H | 0.417 | − | 0.69 | − |
| 994 | Y62W | 1.002 | + | 1.397 | + |
| 995 | L333F | 1.173 | + | 1.17 | + |
| 996 | L181F | 0.367 | − | 0.711 | − |
| 997 | W421F | 1.171 | + | 0.777 | − |
| 998 | L410M | 1.008 | + | 1.09 | + |
| 999 | F392V | 1.32 | + | 1.292 | + |
| 1000 | F345L | 1.023 | + | 1.035 | + |
| 1001 | T400H | 1.326 | + | 1.651 | ++ |
| 1002 | K11R | 1.089 | + | 1.071 | + |
| 1003 | T237F | 1.449 | + | 1.166 | + |
| 1004 | V217C | 1.113 | + | 1.092 | + |
| 1005 | S137K | 0.292 | − | 0.295 | − |
| 1006 | E378D | 1.159 | + | 1.078 | + |
| 1007 | A15R | 1.378 | + | 1.24 | + |
| 1008 | V35M | 1.094 | + | 1.156 | + |
| 1009 | A207T | 1.229 | + | 1.067 | + |
| 1010 | G213K | −0.042 | − | 0.174 | − |
| 1011 | A334M | 1.162 | + | 1.172 | + |
| 1012 | R236I | −0.054 | − | 0.168 | − |
| 1013 | M80I | 1.173 | + | 0.997 | − |
| 1014 | E99T | 1.156 | + | 0.998 | − |
| 1015 | A63E | 1.174 | + | 1.061 | + |
| 1016 | N240T | 1.072 | + | 0.761 | − |
| 1017 | D10N | 0.651 | − | 0.756 | − |
| 1018 | V117L | 1.44 | + | 1.795 | ++ |
| 1019 | V98Y | 1.124 | + | 0.94 | − |
| 1020 | A101R | 0.495 | − | 0.43 | − |
| 1021 | V117T | 1.057 | + | 1.387 | + |
| 1022 | S425W | 0.5 | − | 0.573 | − |
| 1023 | V76M | 0.337 | − | 0.368 | − |
| 1024 | M125A | 0.905 | − | 1.244 | + |
| 1025 | E295D | 1.184 | + | 0.973 | − |
| 1026 | V397Y | 1.15 | + | 1.117 | + |
| 1027 | D55E | 1.189 | + | 1.271 | + |
| 1028 | V136F | 0.448 | − | 0.701 | − |
| 1029 | M125V | 1.16 | + | 1.443 | + |
| 1030 | E362N | 1.199 | + | 1.143 | + |
| 1031 | V100I | 1.475 | + | 1.212 | + |
| 1032 | S183E | 0.97 | − | 0.85 | − |
| 1033 | K335H | 0.868 | − | 0.813 | − |
| 1034 | Q122N | 0.61 | − | 0.691 | − |
| 1035 | V185I | 0.716 | − | 0.898 | − |
| 1036 | I310F | 1.081 | + | 1.084 | + |
| 1037 | M121C | 0.78 | − | 1.135 | + |
| 1038 | K349D | 1.172 | + | 1.082 | + |
| 1039 | T149M | 1.241 | + | 0.975 | − |
| 1040 | K198R | 1.293 | + | 0.996 | − |
| 1041 | S133C | 1.066 | + | 1.036 | + |
| 1042 | L157F | 1.034 | + | 0.974 | − |
| 1043 | M162V | 1.026 | + | 1.034 | + |
| 1044 | C188L | 0.031 | − | 0.29 | − |
| 1045 | S178T | 0.285 | − | 0.617 | − |
| 1046 | N119T | 2.072 | ++ | 0.654 | − |
| 1047 | F392I | 1.583 | ++ | 1.46 | + |
| 1048 | I118S | 0.399 | − | 1.644 | ++ |
| 1049 | Q306E | 1.252 | + | 0.994 | − |
| 1050 | L361V | 0.91 | − | 0.894 | − |
| 1051 | Q122S | 0.847 | − | 0.446 | − |
| 1052 | D447E | 1.11 | + | 1.009 | + |
| 1053 | G108A | 0.244 | − | 0.643 | − |
| 1054 | F238Q | 1.442 | + | 1.015 | + |
| 1055 | Q21R | 1.297 | + | 1.076 | + |
| 1056 | S203A | 1.334 | + | 1.121 | + |
| 1057 | Q212G | 0.629 | − | 0.717 | − |
| 1058 | S202R | 1.093 | + | 1.416 | + |
| 1059 | I118F | 0.1 | − | 0.285 | − |
| 1060 | L264V | 1.255 | + | 0.874 | − |
| 1061 | P297I | 0.843 | − | 0.906 | − |
| 1062 | S75G | 1.228 | + | 1.292 | + |
| 1063 | Y201A | 1.169 | + | 0.818 | − |
| 1064 | K90D | 1.274 | + | 0.98 | − |
| 1065 | T171D | 1.34 | + | 0.976 | − |
| 1066 | N401S | 1.446 | + | 0.99 | − |
| 1067 | S202T | 1.453 | + | 1.137 | + |
| 1068 | S75P | 0.25 | − | 0.826 | − |
| 1069 | N249Y | 1.229 | + | 1.106 | + |
| 1070 | T284V | 1.209 | + | 1.024 | + |
| 1071 | A405V | 1.292 | + | 0.911 | − |
| 1072 | L435M | 1.412 | + | 1.047 | + |
| 1073 | I208Y | 1.129 | + | 1.283 | + |
| 1074 | V197G | 0.275 | − | 0.372 | − |
| 1075 | P27T | 1.386 | + | 1.206 | + |
| 1076 | E378A | 1.15 | + | 1.063 | + |
| 1077 | N61D | 1.104 | + | 0.832 | − |
| 1078 | V215C | 1.375 | + | 1.396 | + |
| 1079 | C418I | 0.183 | − | 0.232 | − |
| 1080 | L187F | 1.201 | + | 1.03 | + |
| 1081 | Y433M | 1.364 | + | 1.203 | + |
| 1082 | A317T | 1.133 | + | 1.108 | + |
| 1083 | I208M | 1.069 | + | 1.102 | + |
| 1084 | C126P | −0.067 | − | 0.175 | − |
| 1085 | M80F | 1.325 | + | 0.81 | − |
| 1086 | P291E | 0.643 | − | 0.634 | − |
| 1087 | I208L | 0.996 | − | 1.035 | + |
| 1088 | K302Q | 1.112 | + | 1.017 | + |
| 1089 | C130A | 1.262 | + | 1.189 | + |
| 1090 | G398T | 0.291 | − | 0.413 | − |
| 1091 | C69H | 0.683 | − | 0.943 | − |
| 1092 | I153V | 1.113 | + | 1.024 | + |
| 1093 | Q248N | 1.308 | + | 1.196 | + |
| 1094 | P291T | 1.172 | + | 1.033 | + |
| 1095 | K234M | 1.165 | + | 0.838 | − |
| 1096 | T237Y | 1.396 | + | 1.235 | + |
| 1097 | T37A | 1.217 | + | 1.197 | + |
| 1098 | S390G | 1.444 | + | 0.951 | − |
| 1099 | I189L | 1.097 | + | 1.211 | − |
| 1100 | Q122G | 0.665 | − | 0.8 | − |
| 1101 | I167F | 1.224 | + | 1.071 | + |
| 1102 | K257H | 0.928 | − | 0.987 | − |
| 1103 | H51R | 0.963 | − | 0.993 | − |
| 1104 | I42M | 1.284 | + | 1.162 | + |
| 1105 | Q28W | 1.04 | + | 0.872 | − |
| 1106 | E155K | 1.041 | + | 1.023 | + |
| 1107 | E47D | 1.237 | + | 1.068 | + |
| 1108 | K83E | 1.118 | + | 0.903 | − |
| 1109 | I426T | 0.653 | − | 0.782 | − |
| 1110 | L160C | 0.984 | − | 0.994 | − |
| 1111 | H51N | 1.092 | + | 1.024 | + |
| 1112 | N115H | 0.252 | − | 0.263 | − |
| 1113 | Q216A | 1.016 | + | 0.657 | − |
| 1114 | K74E | 1.097 | + | 0.616 | − |
| 1115 | N375K | 1.249 | + | 1.038 | + |
| 1116 | S202E | 0.854 | − | 0.728 | − |
| 1117 | I89V | 1.23 | + | 1.016 | + |
| 1118 | R17K | 1.301 | + | 1.03 | + |
| 1119 | T415P | 0.019 | − | 0.165 | − |
| 1120 | S322T | 1.226 | + | 1.078 | + |
| 1121 | H379R | 1.131 | + | 0.966 | − |
| 1122 | G108C | 0.278 | − | 0.397 | − |
| 1123 | K265E | 1.182 | + | 1.199 | + |
| 1124 | T452K | 1.225 | + | 1.064 | + |
| 1125 | D277E | 1.096 | + | 1.058 | + |
| 1126 | Y433E | 0.83 | − | 0.731 | − |
| 1127 | V389A | 1.252 | + | 0.982 | − |
| 1128 | A124M | 1.152 | + | 1.144 | + |
| 1129 | V31R | 0.732 | − | 0.904 | − |
| 1130 | L97V | 1.242 | + | 1.098 | + |
| 1131 | D447N | 1.113 | + | 1.046 | + |
| 1132 | P140E | −0.015 | − | 0.216 | − |
| 1133 | A393L | 0.967 | − | 0.851 | − |
| 1134 | L48R | 0.901 | − | 0.88 | − |
| 1135 | S202Q | 0.912 | − | 1.011 | + |
| 1136 | E247I | −0.022 | − | 0.086 | − |
| 1137 | Q311D | 1.033 | + | 0.972 | − |
| 1138 | L95M | 1.086 | + | 0.968 | − |
| 1139 | Y443F | 1.16 | + | 1.129 | + |
| 1140 | N424R | −0.07 | − | 0.064 | − |
| 1141 | K40D | 0.116 | − | 0.156 | − |
| 1142 | T237C | 1.193 | + | 1.266 | + |
| 1143 | T25E | 1.13 | + | 1.046 | + |
| 1144 | L177I | 1.117 | + | 1.087 | + |
| 1145 | M121I | 1.226 | + | 1.158 | + |
| 1146 | T414V | 1.114 | + | 0.225 | − |
| 1147 | C252I | 1.122 | + | 1.058 | + |
| 1148 | E172H | 1.099 | + | 1.177 | + |
| 1149 | I153C | 1.061 | + | 0.946 | − |
| 1150 | T113Y | 0.132 | − | 0.265 | − |
| 1151 | L95N | 0.984 | − | 0.871 | − |
| 1152 | K257Y | 1.015 | + | 0.965 | − |
| 1153 | I426L | 0.915 | − | 1.309 | + |
| 1154 | V232M | 1.282 | + | 0.999 | − |
| 1155 | M231Q | 1.327 | + | 0.978 | − |
| 1156 | N123P | 0.342 | − | 0.322 | − |
| 1157 | V371W | 0.14 | − | 0.054 | − |
| 1158 | V98I | 1.044 | + | 0.955 | − |
| 1159 | N240C | 0.959 | − | 0.679 | − |
| 1160 | M231R | 1.018 | + | 0.972 | − |
| 1161 | V70M | 1.107 | + | 0.95 | − |
| 1162 | K11E | 1.049 | + | 0.885 | − |
| 1163 | N423G | 1.232 | + | 0.88 | − |
| 1164 | A124I | 1.235 | + | 0.871 | − |
| 1165 | V169M | 1.132 | + | 1.215 | + |
| 1166 | C190P | 0.028 | − | 0.161 | − |
| 1167 | E155D | 1.175 | + | 1.037 | + |
| 1168 | V371S | 1.035 | + | 1.034 | + |
| 1169 | M231I | 1.17 | + | 1.153 | + |
| 1170 | D348K | 1.106 | + | 1.027 | + |
| 1171 | E176D | 1.171 | + | 1.044 | + |
| 1172 | H355S | 1.16 | + | 1.041 | + |
| 1173 | K40E | 0.809 | − | 0.772 | − |
| 1174 | A158V | 1.095 | + | 0.984 | − |
| 1175 | C323P | 1.174 | + | 1.073 | + |
| 1176 | V232N | 0.808 | − | 0.786 | − |
| 1177 | L187I | 1.208 | + | 1.013 | + |
| 1178 | G312T | 1.085 | + | 1.06 | + |
| 1179 | A184G | 1.058 | + | 1.008 | + |
| 1180 | G180Q | 0.864 | − | 0.927 | − |
| 1181 | P377E | 1.318 | + | 1.007 | + |
| 1182 | Y270M | 1.345 | + | 0.991 | − |
| 1183 | K102V | 0.853 | − | 0.528 | − |
| 1184 | P388D | 1.276 | + | 1.191 | + |
| 1185 | Y327F | 1.092 | + | 0.99 | − |
| 1186 | Y26M | 1.019 | + | 0.978 | − |
| 1187 | K349N | 1.145 | + | 1.035 | + |
| 1188 | N429E | 0.118 | − | 0.11 | − |
| 1189 | T284P | 0.808 | − | 0.827 | − |
| 1190 | A4S | 1.316 | + | 1.062 | + |
| 1191 | K349T | 1.149 | + | 1.01 | + |
| 1192 | L49F | 0.747 | − | 1.066 | + |
| 1193 | E455D | 1.105 | + | 1.053 | + |
| 1194 | V110I | 0.648 | − | 0.828 | − |
| 1195 | L187V | 1.242 | + | 0.976 | − |
| 1196 | T413A | 0.603 | − | 0.308 | − |
| 1197 | K33E | 1.045 | + | 0.931 | − |
| 1198 | R440K | 1.276 | + | 1.125 | + |
| 1199 | G312K | 1.155 | + | 1.086 | + |
| 1200 | D64H | 1.071 | + | 1.014 | + |
| 1201 | D407Y | 0.91 | − | 0.928 | − |
| 1202 | V219I | 1.051 | + | 1.049 | + |
| 1203 | C188V | 0.081 | − | 0.586 | − |
| 1204 | R444K | 1.375 | + | 1.109 | + |
| 1205 | K337Q | 0.035 | − | −0.019 | − |
| 1206 | E151Q | 1.162 | + | 1.025 | + |
| 1207 | C323V | 1.288 | + | 1.039 | + |
| 1208 | V120M | 1.112 | + | 1.233 | + |
| 1209 | C190A | 1.242 | + | 1.297 | + |
| 1210 | A199S | 0.823 | − | 0.906 | − |
| 1211 | N240S | 0.584 | − | 0.66 | − |
| 1212 | A263K | 1.129 | + | 1.055 | + |
| 1213 | M182V | 0.801 | − | 0.733 | − |
| 1214 | K164A | 1.089 | + | 0.97 | − |
| 1215 | P388Q | 1.124 | + | 1.133 | + |
| 1216 | L438I | 1.138 | + | 0.997 | − |
| 1217 | G235S | 0.296 | − | 0.604 | − |
| 1218 | T237M | 1.44 | + | 1.572 | ++ |
| 1219 | M436V | 1.147 | + | 1.016 | + |
| 1220 | K294R | 1.475 | + | 1.143 | + |
| 1221 | H434N | 0.888 | − | 0.697 | − |
| 1222 | I66V | 1.275 | + | 1.039 | + |
| 1223 | M12L | 1.012 | + | 0.995 | − |
| 1224 | S75Y | 1.277 | + | 2.108 | ++ |
| 1225 | A334C | 1.215 | + | 1.041 | + |
| 1226 | L97I | 1.444 | + | 1.059 | + |
| 1227 | V5I | 1.059 | + | 0.971 | − |
| 1228 | H51E | 1.45 | + | 1.09 | + |
| 1229 | V215D | 0.499 | − | 0.763 | − |
| 1230 | A158K | 1.587 | ++ | 1.081 | + |
| 1231 | H144R | 1.246 | + | 1.127 | + |
| 1232 | D348S | 1.595 | ++ | 1.029 | + |
| 1233 | T149F | 0.887 | − | 1.088 | + |
| 1234 | E259D | 1.661 | ++ | 1.152 | + |
| 1235 | H51K | 0.953 | − | 0.903 | − |
| 1236 | H368A | 0.169 | − | 0.618 | − |
| 1237 | R391I | −0.01 | − | 0.104 | − |
| 1238 | I387L | 1.008 | + | 1.063 | + |
| 1239 | C109M | 0.413 | − | 0.702 | − |
| 1240 | K129M | 1.173 | + | 1.074 | + |
| 1241 | G180A | 0.946 | − | 0.951 | − |
| 1242 | F345I | 0.999 | − | 0.986 | − |
| 1243 | V117I | 1.256 | + | 1.177 | + |
| 1244 | Q20F | 1.075 | + | 0.949 | − |
| 1245 | G268A | 0.97 | − | 0.865 | − |
| 1246 | E381T | 1.152 | + | 1.083 | + |
| 1247 | P291H | 1.109 | + | 1.056 | + |
| 1248 | L416I | 0.401 | − | 0.56 | − |
| 1249 | K303R | 1.023 | + | 0.894 | − |
| 1250 | A343V | 1.099 | + | 1.302 | + |
| 1251 | A343S | 1.097 | + | 1.089 | + |
| 1252 | Q168N | 1.413 | + | 1.056 | + |
| 1253 | F238Y | 0.951 | − | 1.106 | + |
| 1254 | V147M | 1.419 | + | 1.07 | + |
| 1255 | Y270W | 1.043 | + | 0.99 | − |
| 1256 | A299R | 1.643 | ++ | 1.537 | ++ |
| 1257 | N165D | 1.181 | + | 1.046 | + |
| 1258 | L338M | 0.619 | − | 0.484 | − |
| 1259 | M260F | 0.981 | − | 0.841 | − |
| 1260 | V98M | 1.325 | + | 1.03 | + |
| 1261 | I153M | 1.164 | + | 0.944 | − |
| 1262 | E155Q | 1.288 | + | 1.128 | + |
| 1263 | I167L | 1.084 | + | 0.929 | − |
| 1264 | D82G | 1.114 | + | 1.105 | + |
| 1265 | S174T | 1.512 | ++ | 1.196 | + |
| 1266 | F339C | 0.958 | − | 0.833 | − |
| 1267 | Q122V | 0.292 | − | 0.463 | − |
| 1268 | Y43E | 1.242 | + | 0.57 | − |
| 1269 | E381H | 1.085 | + | 0.998 | − |
| 1270 | M231K | 1.466 | + | 1.301 | + |
| 1271 | S322G | 1.038 | + | 1.002 | + |
| 1272 | I118V | 0.698 | − | 2.437 | ++ |
| 1273 | K90P | 0.583 | − | 0.647 | − |
| 1274 | F339M | 1.398 | + | 0.996 | − |
| 1275 | P291A | 1.056 | + | 0.994 | − |
| 1276 | E247A | 0.359 | − | 0.08 | − |
| 1277 | M175R | 0.724 | − | 1.376 | + |
| 1278 | I18Q | 1.355 | + | 1.126 | + |
| 1279 | N71D | 1.304 | + | 0.381 | − |
| 1280 | I42V | 1.514 | ++ | 1.083 | + |
| 1281 | D352E | 0.933 | − | 0.88 | − |
| 1282 | E254D | 1.214 | + | 1.056 | + |
| 1283 | M448K | 1.082 | + | 1.098 | + |
| 1284 | E2P | 1.277 | + | 1.212 | + |
| 1285 | T324L | 1.257 | + | 1.197 | + |
| 1286 | Q20H | 1.098 | + | 1.005 | + |
| 1287 | A4G | 1.105 | + | 0.989 | − |
| 1288 | P412F | 0.769 | − | 0.689 | − |
| 1289 | V169I | 1.38 | + | 1.391 | + |
| 1290 | Q396D | 1.181 | + | 0.736 | − |
| 1291 | W457F | 1.232 | + | 1.074 | + |
| 1292 | P412R | −0.035 | − | 0.117 | − |
| 1293 | K337E | 0.03 | − | −0.012 | − |
| 1294 | N375T | 1.127 | + | 1.034 | + |
| 1295 | T359E | 1.367 | + | 1.053 | + |
| 1296 | A343P | 0.918 | − | 0.488 | − |
| 1297 | T420S | 1.079 | + | 1.226 | + |
| 1298 | A386R | 1.472 | + | 1.161 | + |
| 1299 | Q122W | 0.121 | − | 0.17 | − |
| 1300 | V150A | 1.262 | + | 1.015 | + |
| 1301 | L166V | 1.272 | + | 1.054 | + |
| 1302 | D44K | 1.327 | + | 1.063 | + |
| 1303 | G39A | 1.326 | + | 1.123 | + |
| 1304 | N401A | 1.574 | ++ | 1.033 | + |
| 1305 | K303E | 1.213 | + | 0.772 | − |
| 1306 | I354C | 1.325 | + | 1.1 | + |
| 1307 | V76L | 0.307 | − | 0.218 | − |
| 1308 | H355E | 1.375 | + | 1.058 | + |
| 1309 | M260L | 1.304 | + | 1.008 | + |
| 1310 | A101H | 1.227 | + | 0.925 | − |
| 1311 | V371Y | −0.018 | − | 0.143 | − |
| 1312 | A199V | 1.098 | + | 1.161 | + |
| 1313 | P138G | 0.628 | − | 0.449 | − |
| 1314 | T420R | −0.04 | − | 0.206 | − |
| 1315 | F392M | 1.215 | + | 1.22 | + |
| 1316 | H51Q | 1.03 | + | 1.134 | + |
| 1317 | T191R | −0.092 | − | 0.113 | − |
| 1318 | I272L | 1.023 | + | 0.975 | − |
| 1319 | N249R | 0.046 | − | −0.029 | − |
| 1320 | R440W | 1.359 | + | 0.75 | − |
| 1321 | A14E | 0.809 | − | 0.793 | − |
| 1322 | N408H | 0.203 | − | 0.175 | − |
| 1323 | V5T | 1.258 | + | 1.109 | + |
| 1324 | V215P | 1.439 | + | 0.819 | − |
| 1325 | Q311E | 1.324 | + | 1.14 | + |
| 1326 | S178G | 0.583 | − | 0.68 | − |
| 1327 | N375R | 1.251 | + | 1.053 | + |
| 1328 | L307M | 1.06 | + | 1.013 | + |
| 1329 | V441I | 1.223 | + | 1.034 | + |
| 1330 | R221D | 1.146 | + | 1.131 | + |
| 1331 | Q216P | 1.046 | + | 0.903 | − |
| 1332 | C418T | 0.912 | − | 0.981 | − |
| 1333 | L160A | 1.119 | + | 1.001 | + |
| 1334 | R221P | 1.121 | + | 1.079 | + |
| 1335 | F286V | 1.264 | + | 1.045 | + |
| 1336 | E259Q | 1.183 | + | 1.079 | + |
| 1337 | L333I | 1.402 | + | 0.873 | − |
| 1338 | S322V | 1.019 | + | 1.013 | + |
| 1339 | Y327V | 1.502 | ++ | 1.081 | + |
| 1340 | E364H | 0.282 | − | 0.595 | − |
| 1341 | A111S | 1.472 | + | 1.12 | + |
| 1342 | E331Y | −0.005 | − | 0.209 | − |
| 1343 | N115Y | −0.083 | − | 0.17 | − |
| 1344 | K68D | 0.487 | − | 0.28 | − |
| 1345 | V441M | 1.146 | + | 0.984 | − |
| 1346 | T415S | 0.995 | − | 0.601 | − |
| 1347 | K84R | 1.129 | + | 0.876 | − |
| 1348 | M406W | 1.124 | + | 0.492 | − |
| 1349 | A179E | 0.568 | − | 0.611 | − |
| 1350 | S374T | 1.212 | + | 1.045 | + |
| 1351 | K68E | 0.079 | − | 0.217 | − |
| 1352 | D407E | 0.956 | − | 1.115 | + |
| 1353 | M363E | 1.069 | + | 0.966 | − |
| 1354 | V389C | 1.206 | + | 1.079 | + |
| 1355 | W77F | 0.901 | − | 1.141 | + |
| 1356 | G328S | 1.003 | + | 0.91 | − |
| 1357 | A393G | 1.007 | + | 1.152 | + |
| 1358 | Q227E | 1.175 | + | 1 | + |
| 1359 | I356V | 1.221 | + | 1.102 | + |
| 1360 | A159E | 0.707 | − | 0.708 | − |
| 1361 | V136I | 1.479 | + | 1.142 | + |
| 1362 | L402M | 1.073 | + | 1.113 | + |
| 1363 | Q306R | 1.389 | + | 1.279 | + |
| 1364 | I320L | 1.132 | + | 1.116 | + |
| 1365 | G180E | 1.031 | + | 0.814 | − |
| 1366 | C190F | −0.048 | − | 0.154 | − |
| 1367 | K40R | 0.694 | − | 0.63 | − |
| 1368 | C323K | 1.124 | + | 1.047 | + |
| 1369 | V76I | 0.713 | − | 1.744 | ++ |
| 1370 | L264F | 1.326 | + | 1.027 | + |
| 1371 | M134V | 0.989 | − | 0.919 | − |
| 1372 | I380V | 1.477 | + | 1.119 | + |
| 1373 | P388E | 1.192 | + | 1.103 | + |
| 1374 | Y296L | 1.366 | + | 1.037 | + |
| 1375 | M448P | 1.302 | + | 1.178 | + |
| 1376 | R325T | 1.37 | + | 1.047 | + |
| 1377 | D55Q | 0.884 | − | 1.263 | + |
| 1378 | K256R | 1.37 | + | 1.138 | + |
| 1379 | S145V | 1.118 | + | 1.504 | ++ |
| 1380 | V301A | 1.422 | + | 1.087 | + |
| 1381 | V217M | 1.216 | + | 0.959 | − |
| 1382 | A263R | 1.512 | ++ | 1.188 | + |
| 1383 | K11Q | 1.108 | + | 1.022 | + |
| 1384 | P291N | 1.062 | + | 1.076 | + |
| 1385 | L342I | 1.088 | + | 0.988 | − |
| 1386 | A405G | 1.115 | + | 0.949 | − |
| 1387 | L187M | 1.158 | + | 0.974 | − |
| 1388 | E99K | 1.471 | + | 1.022 | + |
| 1389 | V370A | 1.62 | ++ | 1.149 | + |
| 1390 | T439K | 0.047 | − | 0.106 | − |
| 1391 | F238W | 0.662 | − | 0.558 | − |
| 1392 | S425A | 1.196 | + | 1.171 | + |
| 1393 | S427A | 1.407 | + | 1.126 | + |
| 1394 | E362S | 1.056 | + | 1.026 | + |
| 1395 | G39R | 0.115 | − | 0.163 | − |
| 1396 | V197A | 0.232 | − | 0.327 | − |
| 1397 | V319Y | 0.18 | − | 0.124 | − |
| 1398 | K302E | 1.288 | + | 1 | + |
| 1399 | K72R | 0.399 | − | 0.766 | − |
| 1400 | L9I | 0.708 | − | 0.831 | − |
| 1401 | K164P | 1.27 | + | 1.067 | + |
| 1402 | G399S | 1.448 | + | 2.561 | ++ |
| 1403 | A41L | 1.003 | + | 1.008 | + |
| 1404 | E385K | 1.453 | + | 1.017 | + |
| 1405 | I261V | 1.293 | + | 0.989 | − |
| 1406 | V147A | 1.453 | + | 0.977 | − |
| 1407 | V152I | 1.311 | + | 1.072 | + |
| 1408 | T245C | 0.515 | − | 0.297 | − |
| 1409 | V389T | 1.229 | + | 0.972 | − |

Key

| | |
|---|---|
| − | lower activity than control <1 |
| + | similar activity to control ≥1 |
| ++ | higher activity than control >1.5 |

### 6.2. Secondary Screen

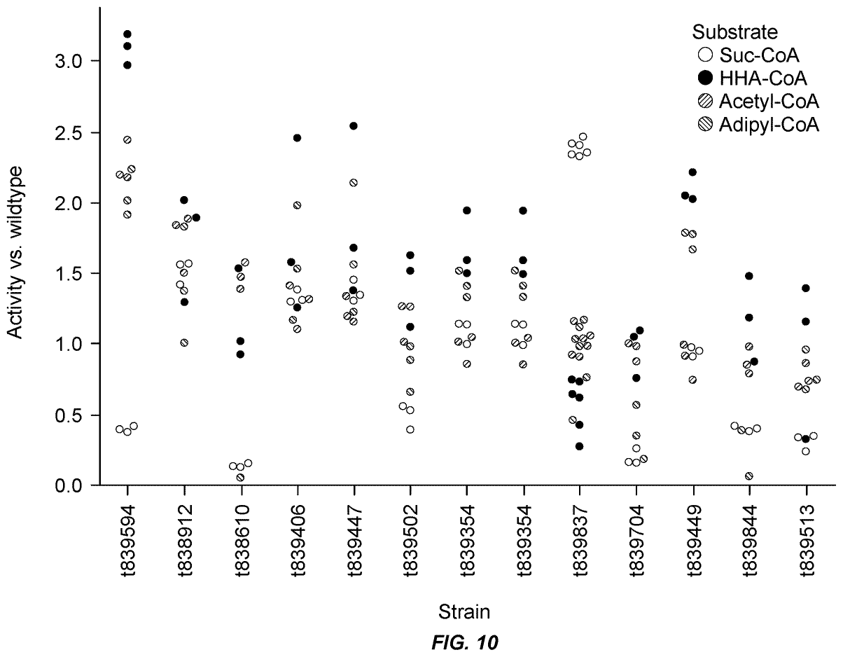

The cultures in the secondary screen went through a similar process as the primary screen and enzymatic activity on HHA-CoA, Suc-CoA, Acetyl-CoA, and Adipyl-CoA were evaluated as described in Example 3.2. While the target substrate is HHA-CoA, activity on Suc-CoA is to be avoided as it results in loss of carbon to 4-HB. Top strains from the secondary screen are shown in FIG. 10.

### Example 7. Pathway Design, Assembly and Small Scale Screening (OSS)

### 7.1. Template Pathway—Primary Screening

Plasmids containing ACLs (homolog 1—SEQ ID NO: 1 and homolog 2—SEQ ID NO: 2) and ALDHs (homolog 3—SEQ ID NO: 3, full data not shown), along with three ADHs with different expression levels were synthesized and transformed into two HHA chassis strains, EChk-1230 and EChk-14936, and screened in a QSSv2 assay.

Briefly, strains carrying the 1,6 HDO pathway (genes encoding the HHA pathway enzymes: 1) a beta ketothiolase, 2) a hydroxyadipyl-CoA dehydrogenase (HBD) 3) a crotonase, 4) trans-enoylCoA reductase (TER), 5) a 6ACA-aldehyde dehydrogenase (ALD) and 6) an alcohol dehydrogenase and the genes encoding the sets) were evaluated in a plate assay and analyzed by LC-MS for 1,6 HDO production.

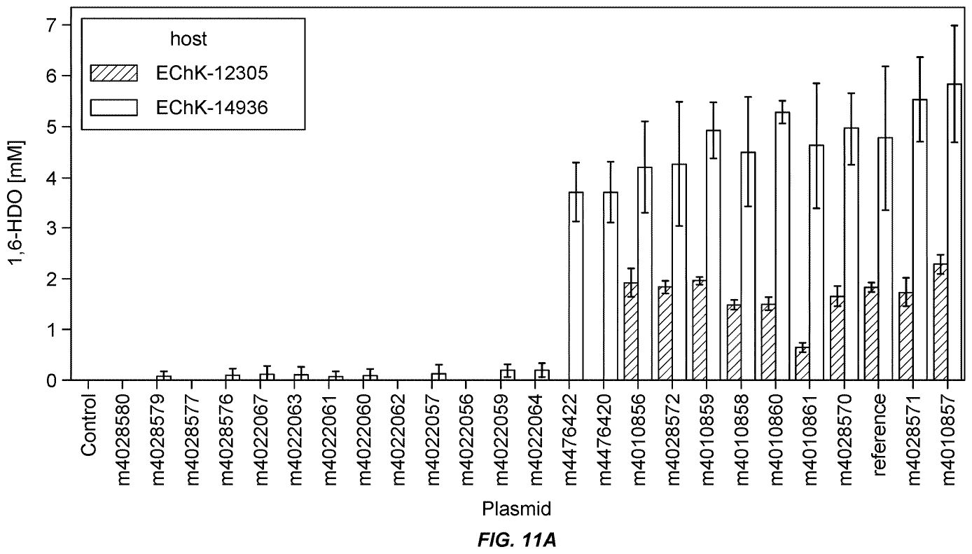

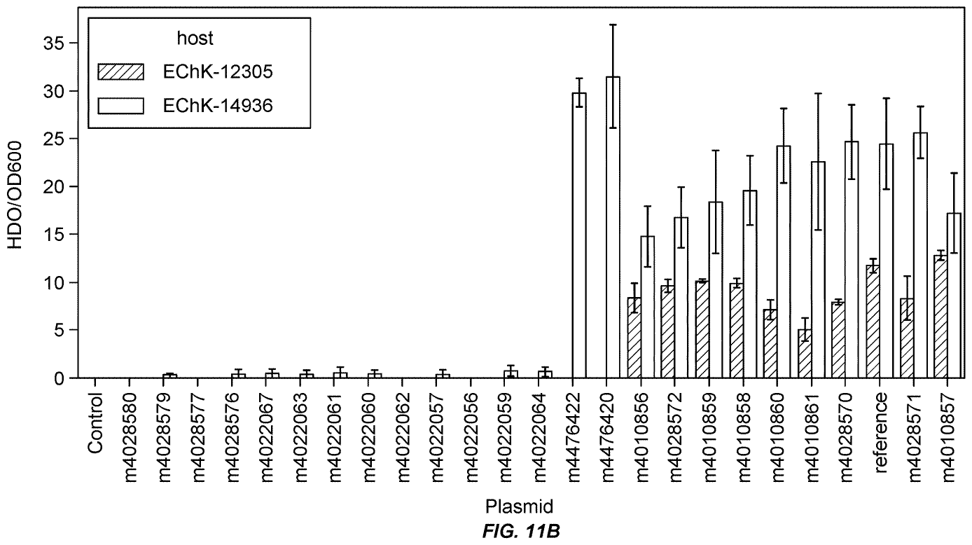

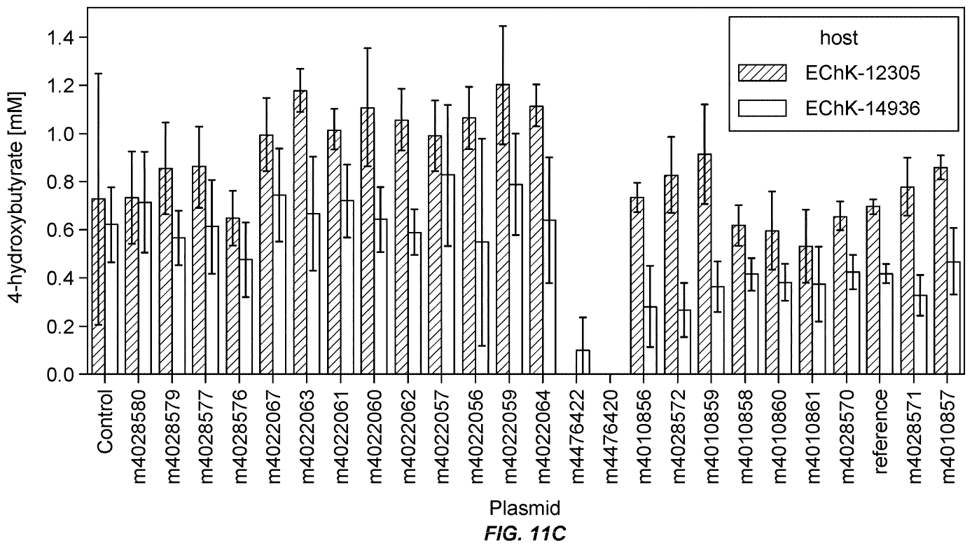

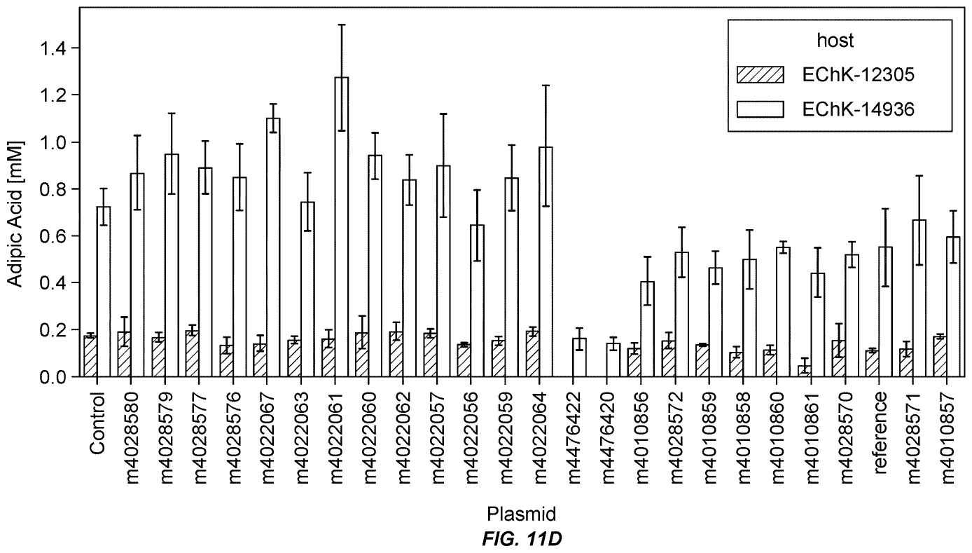

Absolute and relative 1,6-HDO titer from the primary screen are shown in FIGS. 11A and 11B respectively. Relative 1,6 HDO titers are normalized to OD₆₀₀. Control plasmid carries GFP, and the reference plasmid encodes the best performing pathway (data not shown). Higher 1,6-HDO titers were observed when pathway plasmids were transformed into the EChk-14936 HHA production strain which confirms the increased performance of this chassis.

Analysis on this dataset suggested that the ACL enzyme largely impacts 1,6-HDO titers (FIGS. 11A and 11B) and byproduct formation (FIGS. 11C and 11D). The highest 1,6-HDO titers were observed in the majority of plasmids containing the homolog 1 (SEQ ID NO: 1) ACL ligase template.

### 7.2. Template Pathway—Secondary Screening

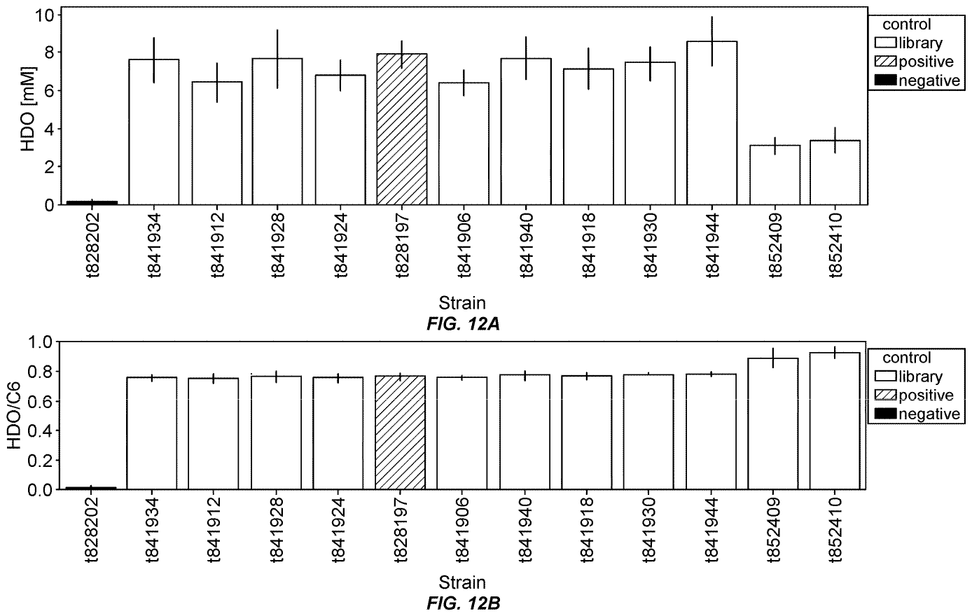

The 12 plasmids in the EChK-14936 strain that converted HHA to 1,6-HDO as seen in FIGS. 11A and 11B were selected for the secondary screening in the QSSv2 model and underwent similar processes as described in Example 7.1. Results from the secondary screen are shown in FIGS. 12A and 12B. As expected, all the strains produced 1,6-HDO (FIG. 12A).

### 7.3. Designing Variant Pathways (Library 3)

A pathway library was designed using best performing variants established in Example 3 and Example 6 using similar processes described in Example 7.1. Expression levels varied for each of ACL variant, ALDH variant and ADH (data not shown). Pathways were cloned in both medium and low-copy plasmids. Library was transformed into the HHA chassis strain EChk-14936 and transformants were screened in a QSSv2 assay in a similar method as described in Example 7.1, using eight replicates of each construct.

### 7.4. Assaying Variant Pathways

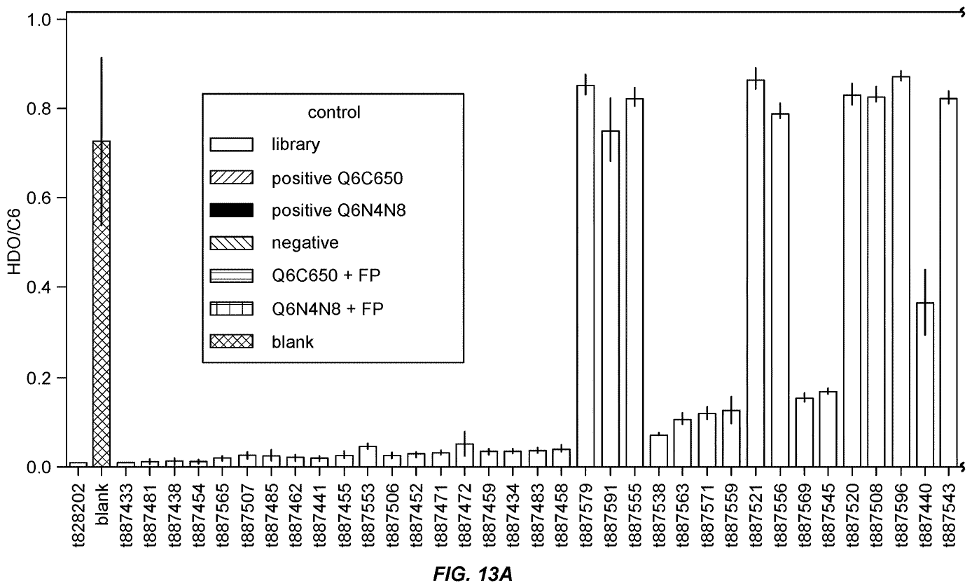

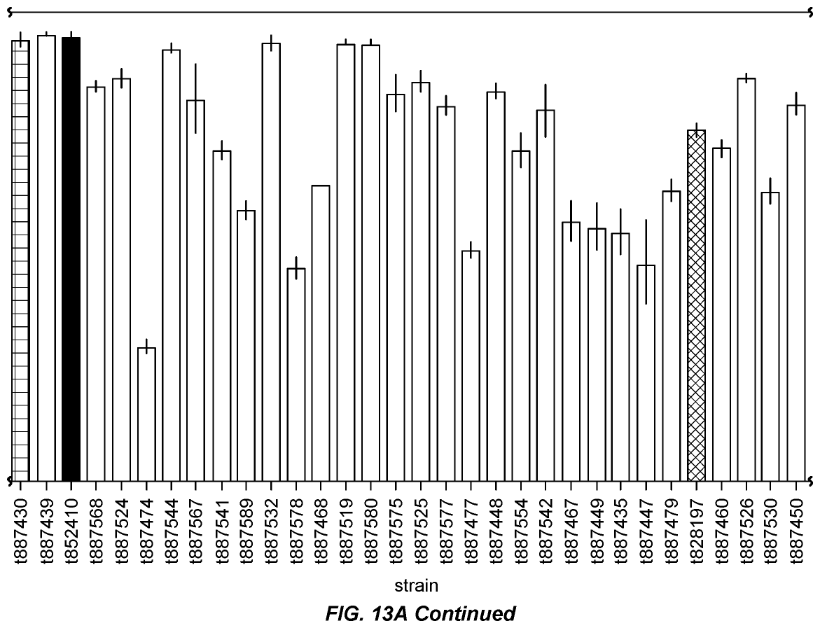

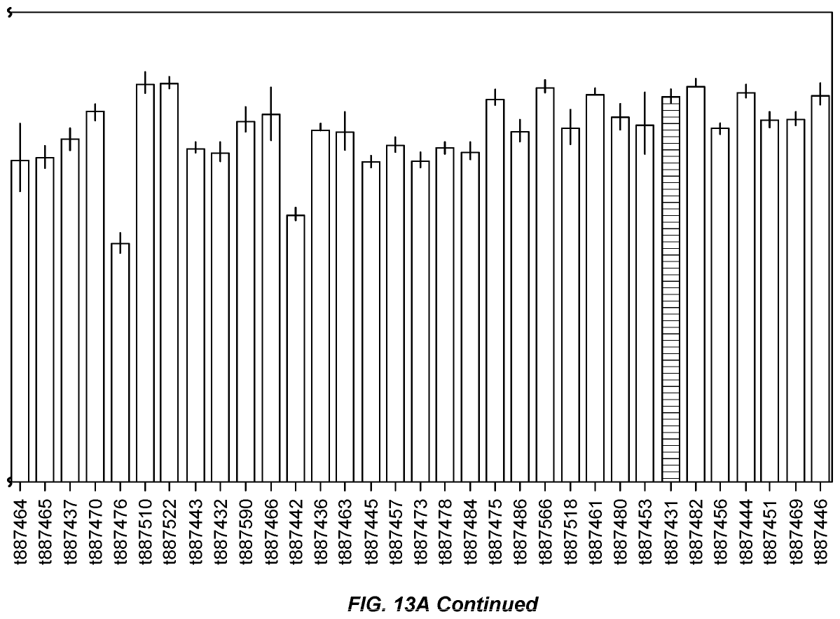

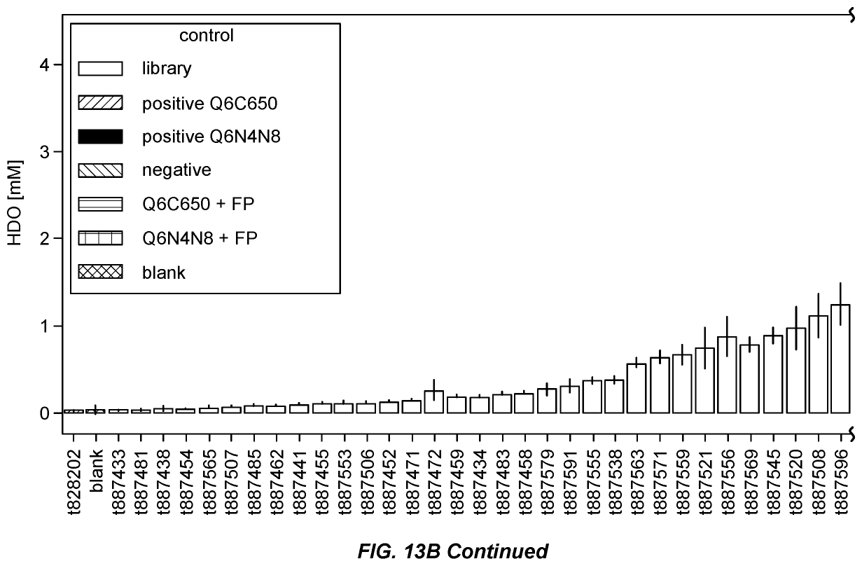

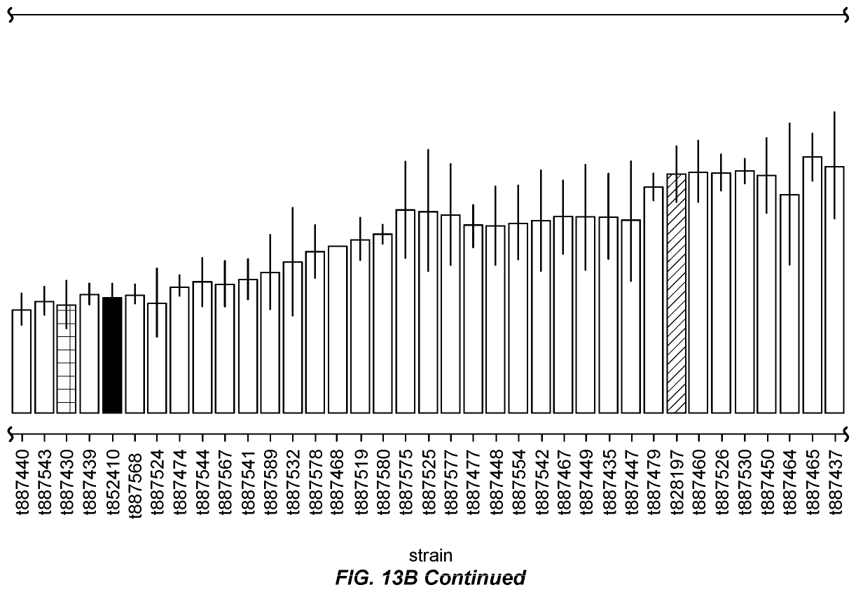

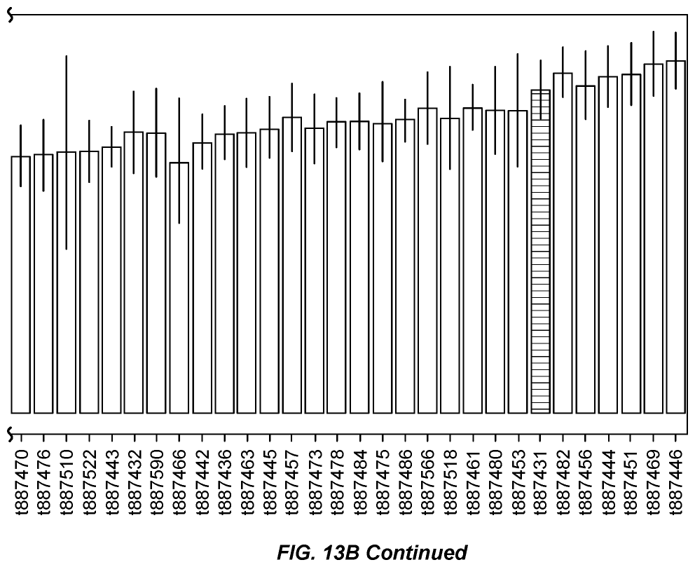

The resulting strains were evaluated in a similar method as described in Example 7.2. FIG. 13A shows the 1,6 HDO conversion of the above pathway and FIG. 13B shows the total HDO titers of the library. Positive controls Q6C650 and Q6N4N8 contained Step G (ACL), H (ALDH) and I (ADH) with the wild-type version of the ACL for comparison with the protein engineered variants

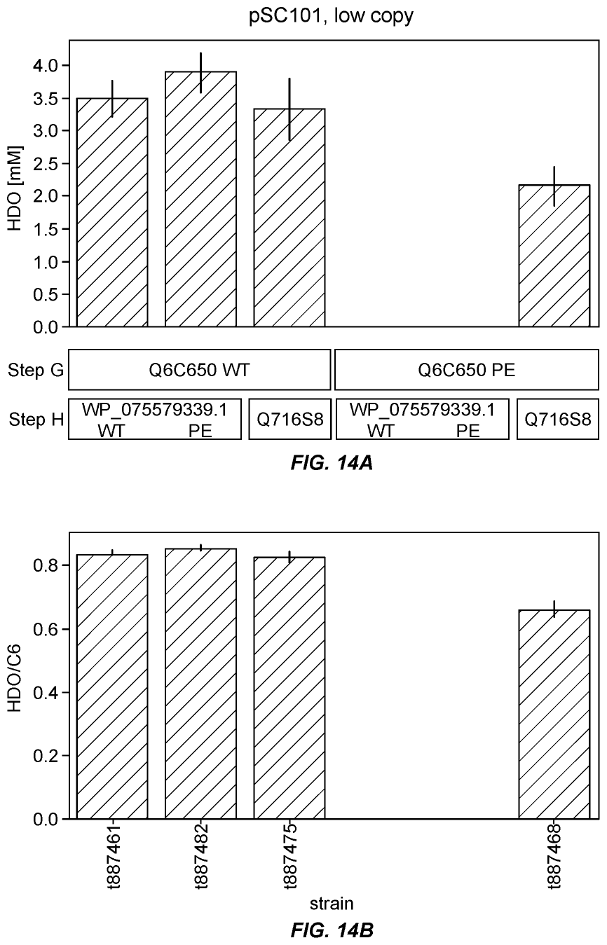

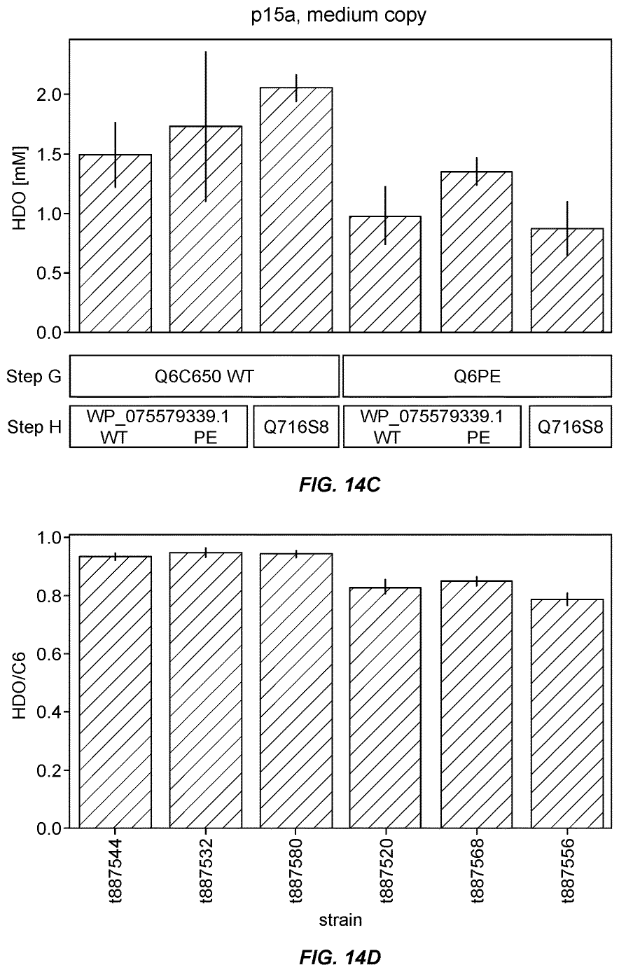

As shown in FIGS. 14A and 14C, expression of the engineered variant of the ALDH enzyme results in similar or higher 1,6-HDO titers when compared to WT (homolog 3—SED ID NO: 3, and WP_075579339.1). However, expression of the engineered variant of the ACL enzyme seems to produce lower 1,6-HDO titers than the WT (homolog 1—SEQ ID NO: 1).

In general, pathways encoded in a lower copy plasmid resulted in higher 1,6-HDO titers (FIGS. 14A and 14B) when compared to pathways encoded in a medium copy plasmid (FIGS. 14C and 14D).

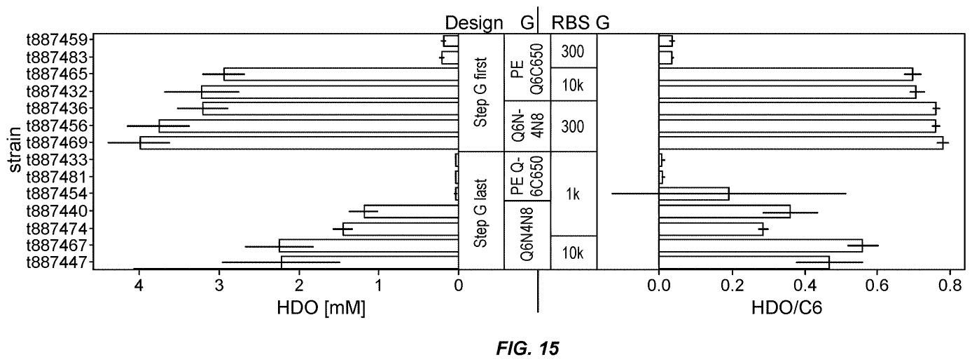

Designs were also assayed where the ACL was moved to the end of the operon and the RBS strengths were varied (FIG. 15). All strains have the HDO pathway enzymes on a low copy vector. Having the ACL gene at the start of the operon resulted in higher titers and better conversion, which is expected since the ACL gene is then closer to the promoter. The weak RBS for the ACL gene only results in significant 1,6-HDO production with the Q6N4N8 enzyme (WT ACL). The Q6C650 strains (homolog 1—SEQ ID NO: 1) perform better when a strong RBS is used.

Based on these results, the following strains were advanced for evaluation in a fermentation assay: t852410 (positive control strain with Q6N4N8—WT ACL), t887430 (FP control with Q6N4N8—WT ACL), t887475 (homolog 1—SEQ ID NO: 1), t887468 (ACL variant of homolog 1), t887461 (homolog 2—SEQ ID NO: 2), and t887482 (ALDH variant of homolog 2).

### Example 8. Fermentation Production of 1,6 HDO

### 8.1. Assaying the Optimized Template Pathway (Library 1)

Hits from Examples 7.1 to 7.2 plus controls, as indicated in TABLE 13, were evaluated in a 12-way Ambr250 fermentation assay.

TABLE 13
Template Pathway Strains Evaluated

| Strain ID | Host | Description |
|---|---|---|
| t828197 | EChK- | Best pathway from Phase I |
| | 14936 | |
| t828202 | EChK- | GFP control |
| | 14936 | |
| t841940 | EChK- | Pathway Library I hit 1 |
| | 14936 | |
| t841944 | EChK- | Pathway Library I hit 2 |
| | 14936 | |

The fermentation process consisted of two phases, seed propagation and production fermentation. The propagation phase aimed to generate metabolically active biomass for inoculation of production fermenters. For the first phase, seed propagation, 5 uL from thawed glycerol stock of the various enzyme-expressing E. coli 1,6-HDO production strain was used as the inoculum to add to 25 mL of LB medium containing 1% glucose and kanamycin (40 mg/L) to 125 mL baffled shake flask. The culture was inoculated at 35° C. and 250-275 rpm for 12 hours in a temperature-controlled incubator shaker. (Stage I). A second flask (Stage II) was prepared by adding 25 mL of FM36 media containing 15 g/L glucose and kanamycin (40 mg/L) in a 125 mL baffled shake flask. 1.5 mL of the overnight culture from the initial flask was used to inoculate the Stage II flask and the flask is incubated at 35° C. with an agitation of 250-275 RPM for 8 hours in a temperature-controlled incubator. After 8 hours, the Stage II flask was used to inoculate 25 mL of FM36 containing 15 g/L glucose and kanamycin (40 mg/L) in a baffled 125 mL flask (Stage III). Stage III was grown at 35° C. with an agitation of 250-275 RPM for 15-16 hrs. The culture volume of stage 3 was modified depending on the number of vessel size needed to be inoculated. The culture volume to headspace ratio was maintained at 1:5. The final target 0D600 was between 3-5. After 15-16 hours, Stage III was used to inoculate a 2 L seed fermenter with approximately 140 mL of culture containing 20 g/L glucose and kanamycin (50 mg/L). A solution of 55% w/w glucose was used as the carbon feed and a one-sided pH control was used to maintain pH at 6.75 using 13.5-14% w/w Ammonia hydroxide. The fermentation was carried out at 35° C. and airflow was maintained at 0.4 VVM based on starting volume.

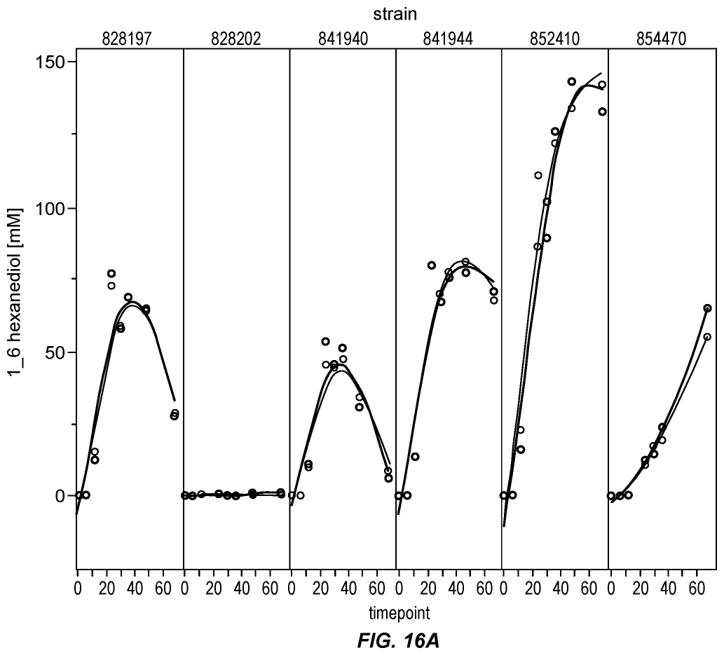

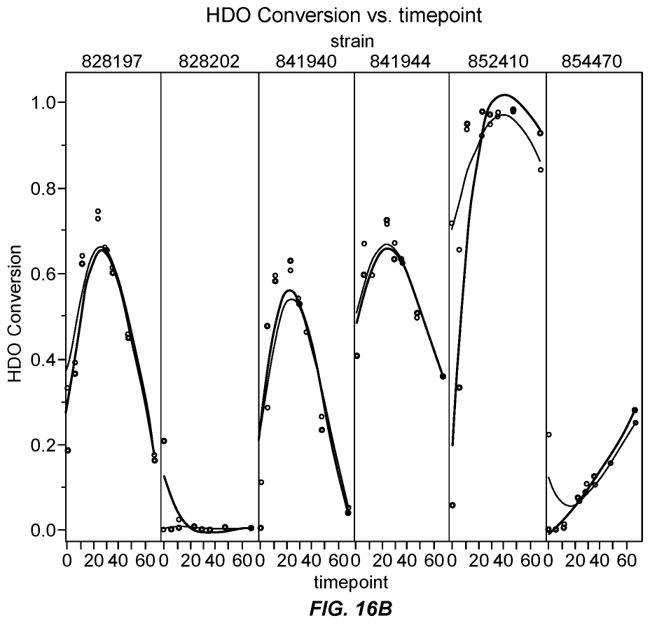

In the growth phase, the reactors were inoculated to an OD of 0.5 using cultures from the stage 3 of the seed train. Several parameters were monitored during the fermentation, which included fermentor exhaust gas composition, dissolved oxygen, residual glucose concentration, and OD of the culture broth. To maintain residual glucose concentration at 40 g/L+/−15 g/L, an off gas-based feed strategy was used. The feed was started 2 hours after a certain setpoint was reached. To adjust this control strategy for the strains tested, different feed multipliers were used. The feed multiplier was set to 1.3 for the first 10 hours of the feed and was then lowered to 1.15 for the remainder of the run. 1.8 mL samples were taken by the automated liquid handler throughout the run and sampled into a chilled (4° C.) 96 deep well plate (timepoints: 0, 6, 12, 24, 30, 36, 48 hr. post inoculation and at the end of fermentation). The samples were processed by spinning down at 4,000 g for 10 mins at 4° C. Supernatant was transferred into a new plate and then aliquoted for different analytical measurements. The total fermentation run length was 60 hours. The results of these fermentation cultures at 60 h are shown in FIGS. 16A and 16B where different colors for each strain representing replicate batches. FIGS. 16A and 16B compares the 1,6-HDO titers and conversion efficiency between the strains.

### 8.2. Assaying the Optimized Variant Pathway (Library 3)

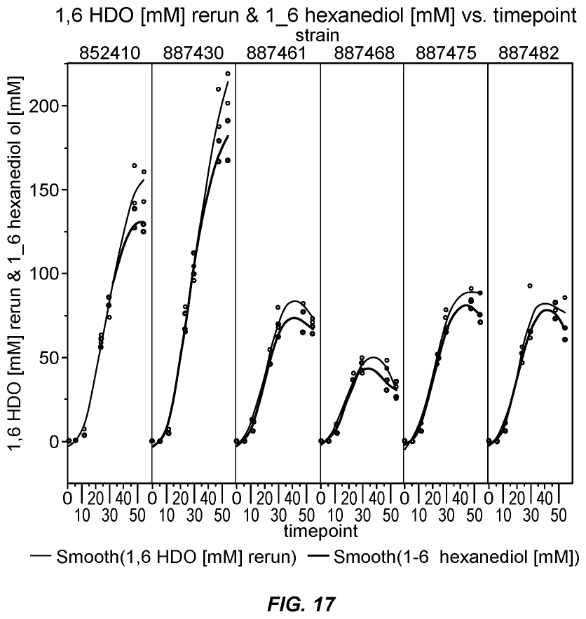

In order to evaluate the impact of the variants and pathways (TABLE 14) identified by the methods described in Example 7 on ability to produce 1,6-HDO during fermentation, a fermentation assay was conducted using the process described in Example 8.1. The results at 60 h are shown in FIG. 17.

TABLE 14
Variant Pathway Strains Evaluated

| Strain ID | ACL | ALDH | ADH | Description |
|---|---|---|---|---|
| t887475 | Homolog 1 | AAT66436.1 | A0A0M8TKC3 | WT ACL |
| | (SEQ ID NO: 1) | | | |
| t887468 | ACL variant | AAT66436.1 | A0A0M8TKC3 | ACL variant |
| | no. 942 of | | | |
| | homolog 1 | | | |
| t887461 | Homolog 1 | Homolog 3 | A0A0M8TKC3 | WT ALDH |
| | (SEQ ID NO: 1) | (SEQ ID NO: 3) | | |
| t887482 | Homolog 1 | ALDH variant | A0A0M8TKC3 | ALDH variant |
| | (SEQ ID NO: 1) | no. 1046 of | | |
| | | homolog 3 | | |

As seen in FIG. 17, introduction of an ACL variant no. 942 of homolog 1 did not improve pathway performance (see strain t887468). In fact, the strain expressing the ACL variant of homolog 1, reached lower 1,6-HDO titers when compared to the strain expressing the WT (homolog 1—SEQ ID NO: 1). Expression of an ALDH variant no. 1046 of homolog 3 did not enhance pathway performance under these conditions (FIG. 17). However, this might be due to the presence of the gene encoding the same enzyme in the genome (step E in the HHA pathway FIG. 1). Swapping the genome integrated variant with the protein engineered variant will grant a better idea on how this variant affects pathway performance.

### Example 9. Genomic Integration of 1,6-HDO Pathway

9.1. Fermentation of Strain Derived from High-Performing Plasmids—Rerun

The below strains were carrying the pathway either on the genome or the plasmid.

TABLE 15
Pathway Strains Evaluated

| Strain ID | Host | Description |
|---|---|---|
| t841944 | EChK-14936 | Pathway 3 (15 g/L 1,6-HDO) |
| t870885 | EChK-14936 | Pathway 3 integrant |
| | aslB::path 3 | |
| t870891 | EChK-14936 | Pathway 3 integrant |
| | arsB::path 3 | |

Strains were evaluated in duplicate in a 12-way fermentation run, and underwent processes as described in Example 8. Strain ID was successfully verified at the end of the fermentation and no mutations were detected in this run.

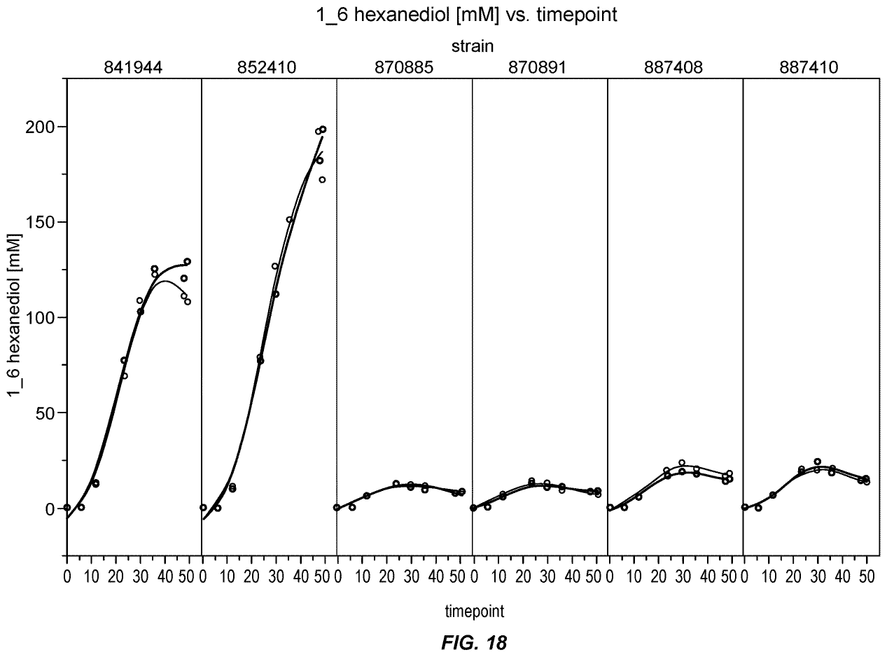

Comparisons in 1,6-HDO titers between all the strains (FIG. 18) show that the genomic integration causes a significant drop in 1,6-HDO titers (~-90%).

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed is:

1. An engineered acyl-coenzyme A ligase (ACL) comprising:
a variant of amino acid sequence SEQ ID NO: 1 or a functional fragment thereof, wherein the engineered ACL comprises one or more alterations at a position described in TABLE 8; or
a variant of amino acid sequence SEQ ID NO: 2 or a functional fragment thereof, wherein the engineered ACL comprises one or more alterations at a position described in TABLE 9.

2. The engineered ACL of claim 1, wherein the engineered ACL is capable of:
a) catalyzing the conversion of 6-hydroxyhexanoate (HHA) to 6-hydroxyhexanoyl coenzyme A (HHA-CoA);
b) catalyzing the conversion of HHA to HHA-CoA at a greater rate compared to wild-type ACL;
c) having a higher specificity for the HHA substrate as compared to the wild type ACL;
d) catalyzing the conversion of 6-aminocaproate (6ACA) to 6-aminocaproyl-CoA (6ACA-CoA);
e) catalyzing the conversion of 6ACA to 6ACA-CoA at a greater rate compared to wild-type ACL;
f) having a higher specificity for the 6ACA substrate as compared to the wild type ACL or g) any combination thereof.

3. The engineered ACL of claim 1 or claim 2, wherein the engineered ACL has higher specificity for conversion of HHA to HHA-CoA, or 6ACA to 6ACA-CoA, or both over conversion of:
a) propionate to propionyl-CoA;
b) acetate to acetyl-CoA;
c) adipate to adipyl-CoA;
d) succinate to succinyl-CoA;
e) 4-hydroxybutyrate (4-HB) to 4-hydroxybutyryl-CoA (4-HBC);
f) 3-hydroxybutyrate (3HB) to 3-hydroxybutyrate-CoA (3HB-CoA);
g) 3-hydroxybutyrate (3HB) to either isomer of 3-hydroxybutyrate-CoA (3HB-CoA); or
h) any combination thereof.

4. The engineered ACL of any one of claims 1 to 3, wherein the engineered ACL has higher specificity for conversion of adipate to adipyl-CoA, succinate to succinyl-CoA, or both, over conversion of 4-HB to 4-HBC.

5. The engineered ACL of any one of claims 1 to 4, wherein the engineered ACL comprises an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an ACL consisting of the amino acid sequence of SEQ ID NO: 1 or SEQ ID NO: 2.

6. The engineered ACL of any one of claims 1 to 5, wherein the one or more amino acid alterations comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 or more alterations.

7. The engineered ACL of any one of claims 1 to 6, wherein the one or more amino acid alterations are conservative or non-conservative amino acid substitutions, deletions, or insertions, or any combination thereof.

8. The engineered ACL of any one of claims 1 to 7, wherein the ACL comprises one or more amino acid alterations at a position corresponding to position 4, 7, 8, 9, 10, 12, 13, 15, 16, 19, 20, 22, 30, 31, 35, 36, 39, 40, 43, 44, 47, 48, 49, 50, 51, 52, 53, 54, 55, 60, 61, 64, 66, 67, 69, 72, 73, 74, 76, 78, 80, 81, 82, 83, 86, 87, 88, 89, 90, 91, 94, 95, 96, 97, 98, 99, 100, 101, 102, 103, 104, 105, 106, 107, 109, 111, 112, 113, 114, 115, 116, 117, 119, 121, 126, 127, 128, 129, 130, 131, 136, 137, 138, 139, 140, 144, 149, 150, 152, 153, 159, 162, 163, 164, 165, 166, 167, 170, 172, 182, 189, 191, 194, 195, 196, 197, 198, 199, 200, 201, 202, 205, 208, 209, 211, 212, 213, 214, 215, 217, 220, 221, 222, 223, 224, 225, 226, 227, 228, 229, 230, 231, 232, 233, 234, 235, 238, 239, 240, 241, 242, 243, 244, 246, 247, 248, 249, 250, 251, 252, 253, 254, 255, 256, 257, 258, 259, 260, 261, 262, 264, 265, 266, 267, 268, 269, 270, 271, 272, 274, 275, 276, 277, 278, 279, 280, 281, 282, 286, 287, 288, 289, 290, 291, 294, 295, 296, 297, 298, 299, 300, 304, 306, 309, 310, 311, 314, 315, 316, 317, 318, 319, 320, 321, 323, 324, 326, 327, 328, 329, 330, 331, 334, 335, 336, 338, 339, 341, 342, 348, 351, 352, 353, 354, 355, 356, 357, 359, 360, 362, 363, 364, 365, 366, 367, 368, 369, 370, 371, 372, 373, 374, 375, 376, 377, 378, 379, 380, 381, 383, 384, 386, 387, 388, 389, 390, 391, 394, 398, 402, 406, 407, 410, 411, 412, 413, 414, 415, 416, 418, 419, 420, 424, 425, 426, 430, 431, 432, 433, 436, 442, 443, 444, 445, 446, 447, 455, 459, 461, 462, 464, 465, 469, 470, 471, 472, 474, 475, 476, 477, 478, 479, 480, 481, 484, 486, 489, 490, 491, 493, 494, 496, 503, 506, 507, 508, 509, 513, 518, 519, 521, 523, 524, 525, 527, 528, 529, 539, 540, 542, 546, 551, 553, 554, 555, 557, 559, 560, 562, 564, 565, 566, 583, or a combination thereof, in SEQ ID NO: 1.

9. The engineered ACL of any one of claims 1 to 8, wherein the ACL comprises one or more amino acid alterations at a position corresponding to position 7, 8, 10, 12, 15, 16, 19, 52, 54, 64, 66, 67, 73, 74, 82, 87, 90, 94, 100, 111, 115, 117, 119, 121, 129, 131, 138, 139, 140, 144, 150, 165, 166, 172, 182, 191, 199, 209, 212, 217, 221, 223, 224, 227, 229, 230, 231, 232, 233, 234, 235, 238, 240, 243, 249, 253, 254, 256, 258, 259, 262, 268, 271, 275, 277, 281, 288, 290, 296, 297, 300, 304, 306, 316, 318, 320, 321, 323, 328, 330, 331, 336, 352, 353, 363, 364, 365, 366, 369, 372, 374, 376, 378, 388, 390, 394, 398, 406, 407, 416, 432, 443, 445, 455, 462, 469, 470, 471, 480, 481, 484, 486, 490, 491, 493, 494, 496, 508, 513, 518, 519, 521, 523, 525, 546, 554, 555, 560, 565, 583 or a combination thereof, in SEQ ID NO: 1.

10. The engineered ACL of any one of claims 1 to 9, wherein the ACL comprises one or more amino acid alterations at a position corresponding to position 10, 199, 223, 229, 230, 231, 232, 233, 234, 235, 238, 243, 256, 258, 277, 288, 300, 318, 320, 321, 323, 328, 330, 364, 365, 369, 372, 374, 378, 462, 469, 480, 481, 484, 513, 523, 525, 554, 560, 583 or any combination thereof, in SEQ ID NO: 1.

11. The engineered ACL of any one of claims 1 to 10, wherein the ACL comprises one or more amino acid alterations at a position corresponding to position 223, 232, 233, 234, 238, 258, 288, 318, 320, 321, 323, 364, 513, 523 or any combination thereof, in SEQ ID NO: 1.

12. The engineered ACL of any one of claims 1 to 11, wherein the one or more amino acid alterations result in an engineered ACL comprising:
a) M at a residue corresponding to position 4 in SEQ ID NO: 1;
b) D, K, Q, S, T at a residue corresponding to position 7 in SEQ ID NO: 1;
c) I, S at a residue corresponding to position 8 in SEQ ID NO: 1;
d) A, K, S at a residue corresponding to position 9 in SEQ ID NO: 1;
e) F, H, P, V, W, Y at a residue corresponding to position 10 in SEQ ID NO: 1;
f) D, E, H, P at a residue corresponding to position 12 in SEQ ID NO: 1;
g) C, M at a residue corresponding to position 13 in SEQ ID NO: 1;
h) D, I, L, P at a residue corresponding to position 15 in SEQ ID NO: 1;
i) E, H, N at a residue corresponding to position 16 in SEQ ID NO: 1;
j) F, L, P, Q at a residue corresponding to position 19 in SEQ ID NO: 1;
k) W at a residue corresponding to position 20 in SEQ ID NO: 1;
l) S at a residue corresponding to position 22 in SEQ ID NO: 1;
m) D at a residue corresponding to position 30 in SEQ ID NO: 1;
n) A at a residue corresponding to position 31 in SEQ ID NO: 1;
o) G, P at a residue corresponding to position 35 in SEQ ID NO: 1;
p) H at a residue corresponding to position 36 in SEQ ID NO: 1;
q) A at a residue corresponding to position 39 in SEQ ID NO: 1;
r) C, F, L, M at a residue corresponding to position 40 in SEQ ID NO: 1;
s) G at a residue corresponding to position 43 in SEQ ID NO: 1;
t) D, H, S, T at a residue corresponding to position 44 in SEQ ID NO: 1;
u) R, RTYTQKEL (SEQ ID NO: 15), RTYTQREL (SEQ ID NO: 16), RTYT (SEQ ID NO: 17) at a residue corresponding to position 47 in SEQ ID NO: 1;
v) a deletion at a residue corresponding to position 48 in SEQ ID NO: 1;
w) a deletion, Y at a residue corresponding to position 49 in SEQ ID NO: 1;
x) a deletion, T at a residue corresponding to position 50 in SEQ ID NO: 1;
y) a deletion, F, H, L, R, W, Y at a residue corresponding to position 51 in SEQ ID NO: 1;
z) a deletion, K, R at a residue corresponding to position 52 in SEQ ID NO: 1;
aa) a deletion at a residue corresponding to position 53 in SEQ ID NO: 1;
bb) a deletion, L at a residue corresponding to position 54 in SEQ ID NO: 1;
cc) L at a residue corresponding to position 55 in SEQ ID NO: 1;
dd) R at a residue corresponding to position 60 in SEQ ID NO: 1;
ee) F, L at a residue corresponding to position 61 in SEQ ID NO: 1;
ff) A, G at a residue corresponding to position 64 in SEQ ID NO: 1;
gg) R at a residue corresponding to position 66 in SEQ ID NO: 1;
hh) A at a residue corresponding to position 67 in SEQ ID NO: 1;
ii) L at a residue corresponding to position 69 in SEQ ID NO: 1;
jj) G, T at a residue corresponding to position 72 in SEQ ID NO: 1;
kk) F, G, L, V at a residue corresponding to position 73 in SEQ ID NO: 1;
ll) K at a residue corresponding to position 74 in SEQ ID NO: 1;
mm) T at a residue corresponding to position 76 in SEQ ID NO: 1;
nn) V at a residue corresponding to position 78 in SEQ ID NO: 1;
oo) G at a residue corresponding to position 80 in SEQ ID NO: 1;
pp) PG at a residue corresponding to position 81 in SEQ ID NO: 1;
qq) A, E, G, N, R, T at a residue corresponding to position 82 in SEQ ID NO: 1;
rr) a deletion, K, P, R, S at a residue corresponding to position 83 in SEQ ID NO: 1;
ss) I at a residue corresponding to position 86 in SEQ ID NO: 1;
tt) I, V, VAV at a residue corresponding to position 87 in SEQ ID NO: 1;
uu) a deletion, A at a residue corresponding to position 88 in SEQ ID NO: 1;
vv) a deletion, I, L, V at a residue corresponding to position 89 in SEQ ID NO: 1;
ww) C, L, M, V, W at a residue corresponding to position 90 in SEQ ID NO: 1;
xx) A, L, M, S at a residue corresponding to position 91 in SEQ ID NO: 1;
yy) C, CIEAA (SEQ ID NO: 18), S, T, TIEAA (SEQ ID NO: 19) at a residue corresponding to position 94 in SEQ ID NO: 1;
zz) a deletion at a residue corresponding to position 95 in SEQ ID NO: 1;
aaa) a deletion, E at a residue corresponding to position 96 in SEQ ID NO: 1;
bbb) a deletion at a residue corresponding to position 97 in SEQ ID NO: 1;
ccc) a deletion, A, L at a residue corresponding to position 98 in SEQ ID NO: 1;
ddd) C, M at a residue corresponding to position 99 in SEQ ID NO: 1;
eee) S, M, L at a residue corresponding to position 100 in SEQ ID NO: 1;
fff) F, M at a residue corresponding to position 101 in SEQ ID NO: 1;
ggg) H, F, L at a residue corresponding to position 102 in SEQ ID NO: 1;
hhh) G at a residue corresponding to position 103 in SEQ ID NO: 1;
iii) V, L, T at a residue corresponding to position 104 in SEQ ID NO: 1;
jjj) V, W, H, A, I at a residue corresponding to position 105 in SEQ ID NO: 1;
kkk) S, Y, R, K at a residue corresponding to position 106 in SEQ ID NO: 1;
lll) A, C, V at a residue corresponding to position 107 in SEQ ID NO: 1;
mmm) L, V, Q at a residue corresponding to position 109 in SEQ ID NO: 1;
nnn) V, A, M, L, C, VTPAN (SEQ ID NO: 20) at a residue corresponding to position 111 in SEQ ID NO: 1;
ooo) T, a deletion at a residue corresponding to position 112 in SEQ ID NO: 1;
ppp) a deletion, L at a residue corresponding to position 113 in SEQ ID NO: 1;
qqq) a deletion, A, L, V at a residue corresponding to position 114 in SEQ ID NO: 1;
rrr) a deletion, N at a residue corresponding to position 115 in SEQ ID NO: 1;
sss) A, W, P at a residue corresponding to position 116 in SEQ ID NO: 1;
ttt) S at a residue corresponding to position 117 in SEQ ID NO: 1;
uuu) T at a residue corresponding to position 119 in SEQ ID NO: 1;
vvv) R, D at a residue corresponding to position 121 in SEQ ID NO: 1;
www) H at a residue corresponding to position 126 in SEQ ID NO: 1;
xxx) M, L, MRHTG (SEQ ID NO: 21), MRITG (SEQ ID NO: 22) at a residue corresponding to position 127 in SEQ ID NO: 1;
yyy) a deletion at a residue corresponding to position 128 in SEQ ID NO: 1;
zzz) a deletion, I, H at a residue corresponding to position 129 in SEQ ID NO: 1;
aaaa) a deletion at a residue corresponding to position 130 in SEQ ID NO: 1;
bbbb) a deletion, G, D at a residue corresponding to position 131 in SEQ ID NO: 1;
cccc) L at a residue corresponding to position 136 in SEQ ID NO: 1;
dddd) T at a residue corresponding to position 137 in SEQ ID NO: 1;
eeee) D, S at a residue corresponding to position 138 in SEQ ID NO: 1;
ffff) E at a residue corresponding to position 139 in SEQ ID NO: 1;
gggg) E, K, D at a residue corresponding to position 140 in SEQ ID NO: 1;
hhhh) T at a residue corresponding to position 144 in SEQ ID NO: 1;
iiii) R at a residue corresponding to position 149 in SEQ ID NO: 1;
jjjj) I, L at a residue corresponding to position 150 in SEQ ID NO: 1;
kkkk) A, T, C, I at a residue corresponding to position 152 in SEQ ID NO: 1;
llll) G, S at a residue corresponding to position 153 in SEQ ID NO: 1;
mmmm) G at a residue corresponding to position 159 in SEQ ID NO: 1;
nnnn) S, H, R, SIDAF (SEQ ID NO: 23), RIESF (SEQ ID NO: 24), HFDSF (SEQ ID NO: 25), HFDAF (SEQ ID NO: 26), HIDSF (SEQ ID NO: 27) at a residue corresponding to position 162 in SEQ ID NO: 1;
oooo) F, I, a deletion at a residue corresponding to position 163 in SEQ ID NO: 1;
pppp) a deletion, D at a residue corresponding to position 164 in SEQ ID NO: 1;
qqqq) a deletion, S at a residue corresponding to position 165 in SEQ ID NO: 1;
rrrr) a deletion, F at a residue corresponding to position 166 in SEQ ID NO: 1;
ssss) L at a residue corresponding to position 167 in SEQ ID NO: 1;
tttt) G at a residue corresponding to position 170 in SEQ ID NO: 1;
uuuu) L, A, D at a residue corresponding to position 172 in SEQ ID NO: 1;
vvvv) Q at a residue corresponding to position 182 in SEQ ID NO: 1;
wwww) E, G at a residue corresponding to position 189 in SEQ ID NO: 1;
xxxx) A at a residue corresponding to position 191 in SEQ ID NO: 1;
yyyy) D at a residue corresponding to position 194 in SEQ ID NO: 1;
zzzz) T, P, M at a residue corresponding to position 195 in SEQ ID NO: 1;
aaaaa) T, I at a residue corresponding to position 196 in SEQ ID NO: 1;
bbbbb) W, C at a residue corresponding to position 197 in SEQ ID NO: 1;
ccccc) Y, V, S, W, I, M, T at a residue corresponding to position 198 in SEQ ID NO: 1;
ddddd) M, L, W, P, F, C, A at a residue corresponding to position 199 in SEQ ID NO: 1;
eeeee) W at a residue corresponding to position 200 in SEQ ID NO: 1;
fffff) F, T, N, P, Y, L at a residue corresponding to position 201 in SEQ ID NO: 1;
ggggg) T, H, A, Q, E, L, G at a residue corresponding to position 202 in SEQ ID NO: 1;
hhhhh) T at a residue corresponding to position 205 in SEQ ID NO: 1;
iiiii) F at a residue corresponding to position 208 in SEQ ID NO: 1;
jjjjj) P at a residue corresponding to position 209 in SEQ ID NO: 1;
kkkkk) G at a residue corresponding to position 211 in SEQ ID NO: 1;
lllll) A, C at a residue corresponding to position 212 in SEQ ID NO: 1;
mmmmm) M, V, E at a residue corresponding to position 213 in SEQ ID NO: 1;
nnnnn) V at a residue corresponding to position 214 in SEQ ID NO: 1;
ooooo) S at a residue corresponding to position 215 in SEQ ID NO: 1;
ppppp) Q, R, Y, H, K, G at a residue corresponding to position 217 in SEQ ID NO: 1;
qqqqq) W, M, V at a residue corresponding to position 220 in SEQ ID NO: 1;
rrrrr) T, A at a residue corresponding to position 221 in SEQ ID NO: 1;
sssss) S, N at a residue corresponding to position 222 in SEQ ID NO: 1;
ttttt) A, N, I, L, H at a residue corresponding to position 223 in SEQ ID NO: 1;
uuuuu) T, Q, H, I, N, M at a residue corresponding to position 224 in SEQ ID NO: 1;
vvvvv) L, A, F, Q at a residue corresponding to position 225 in SEQ ID NO: 1;
wwwww) Y, C, T, W at a residue corresponding to position 226 in SEQ ID NO: 1;
xxxxx) G, E, M, T, A, GKWSQD (SEQ ID NO: 28), GKWSQSY (SEQ ID NO: 29), GKWSQ (SEQ ID NO: 30), GKWSQDLK (SEQ ID NO: 31), GKWSQQLQ (SEQ ID NO: 32) at a residue corresponding to position 227 in SEQ ID NO: 1;
yyyyy) a deletion at a residue corresponding to position 228 in SEQ ID NO: 1;
zzzzz) a deletion, F, S, L, G, W, I, M, H at a residue corresponding to position 229 in SEQ ID NO: 1;
aaaaaa) a deletion, R, M, P, T, E, Q, A at a residue corresponding to position 230 in SEQ ID NO: 1;
bbbbbb) a deletion, Q, P, H, QDLQ (SEQ ID NO: 33) at a residue corresponding to position 231 in SEQ ID NO: 1;
cccccc) a deletion, W, A, Y, N, K, Q, D at a residue corresponding to position 232 in SEQ ID NO: 1;
dddddd) a deletion, W, R, M, Y, P at a residue corresponding to position 233 in SEQ ID NO: 1;
eeeeee) a deletion, F, Y, T, K, R, Q, P, G, H, E at a residue corresponding to position 234 in SEQ ID NO: 1;
ffffff) H, S, R, K, M, Q, D, G at a residue corresponding to position 235 in SEQ ID NO: 1;
gggggg) P, N, H, T, D, DISAC (SEQ ID NO: 34), DVS at a residue corresponding to position 238 in SEQ ID NO: 1;
hhhhhh) V, F, R, a deletion at a residue corresponding to position 239 in SEQ ID NO: 1;
iiiiii) a deletion, F, H, T, W, S, L, C, V at a residue corresponding to position 240 in SEQ ID NO: 1;
jjjjjj) a deletion, C, M, P, I at a residue corresponding to position 241 in SEQ ID NO: 1;
kkkkkk) a deletion, G, V, C at a residue corresponding to position 242 in SEQ ID NO: 1;
llllll) L, C, M at a residue corresponding to position 243 in SEQ ID NO: 1;
mmmmmm) M, V at a residue corresponding to position 244 in SEQ ID NO: 1;
nnnnnn) W, F, L, Y at a residue corresponding to position 246 in SEQ ID NO: 1;
oooooo) F, C at a residue corresponding to position 247 in SEQ ID NO: 1;
pppppp) S, P at a residue corresponding to position 248 in SEQ ID NO: 1;
qqqqqq) A, V, M at a residue corresponding to position 249 in SEQ ID NO: 1;
rrrrrr) M, A, W at a residue corresponding to position 250 in SEQ ID NO: 1;
ssssss) A, H at a residue corresponding to position 251 in SEQ ID NO: 1;
tttttt) I, M, W, Q at a residue corresponding to position 252 in SEQ ID NO: 1;
uuuuuu) T, Q, N, I, H, V, M at a residue corresponding to position 253 in SEQ ID NO: 1;
vvvvvv) E, I, T, Y, H, V, L, P, A, M, F, S, G at a residue corresponding to position 254 in SEQ ID NO: 1;
wwwwww) M, T, I, N, Y, C, ILLANV (SEQ ID NO: 35), ILLANVHY (SEQ ID NO: 36), ILLLNVHY (SEQ ID NO: 37), ILLLNV (SEQ ID NO: 38), ILLLNVHK (SEQ ID NO: 39), ILLHNVHK (SEQ ID NO: 40), ILLSNVHY (SEQ ID NO: 41) at a residue corresponding to position 255 in SEQ ID NO: 1;
xxxxxx) I, V, H, C, F, a deletion at a residue corresponding to position 256 in SEQ ID NO: 1;
yyyyyy) a deletion, L, T, N at a residue corresponding to position 257 in SEQ ID NO: 1;
zzzzzz) a deletion, S, L, P, M, H, T, A at a residue corresponding to position 258 in SEQ ID NO: 1;
aaaaaaa) a deletion, C, A at a residue corresponding to position 259 in SEQ ID NO: 1;
bbbbbbb) a deletion, F, L, M, V at a residue corresponding to position 260 in SEQ ID NO: 1;
ccccccc) a deletion, Y, M, W, A, F, L, S at a residue corresponding to position 261 in SEQ ID NO: 1;
ddddddd) a deletion, M, K, Y, R, G, C, L, V, S, N at a residue corresponding to position 262 in SEQ ID NO: 1;
eeeeeee) G, W, I, S, V at a residue corresponding to position 264 in SEQ ID NO: 1;
fffffff) R, C, H, K at a residue corresponding to position 265 in SEQ ID NO: 1;
ggggggg) I, V, Q, C at a residue corresponding to position 266 in SEQ ID NO: 1;
hhhhhhh) Y at a residue corresponding to position 267 in SEQ ID NO: 1;
iiiiiii) V, M, T, F, IPR (SEQ ID NO: 42), EVINR (SEQ ID NO: 43) at a residue corresponding to position 268 in SEQ ID NO: 1;
jjjjjjj) M, at a residue corresponding to position 269 in SEQ ID NO: 1;
kkkkkkk) a deletion, P at a residue corresponding to position 270 in SEQ ID NO: 1;
lllllll) a deletion, R, Q at a residue corresponding to position 271 in SEQ ID NO: 1;
mmmmmmm) W at a residue corresponding to position 272 in SEQ ID NO: 1;
nnnnnnn) P, PEEMLRAIQ (SEQ ID NO: 44), PEEMLQAIQ (SEQ ID NO: 45) at a residue corresponding to position 274 in SEQ ID NO: 1;
ooooooo) D, A, E, R, K, EEMLQAIQ (SEQ ID NO: 46), EEMLRAIQ (SEQ ID NO: 47), a deletion, KEMLQAIQ (SEQ ID NO: 48) at a residue corresponding to position 275 in SEQ ID NO: 1;
ppppppp) a deletion at a residue corresponding to position 276 in SEQ ID NO: 1;
qqqqqqq) a deletion, M, Y, L, F at a residue corresponding to position 277 in SEQ ID NO: 1;
rrrrrrr) a deletion at a residue corresponding to position 278 in SEQ ID NO: 1;
sssssss) a deletion, Q, R, QAIQ (SEQ ID NO: 49), RAIQ (SEQ ID NO: 50) at a residue corresponding to position 279 in SEQ ID NO: 1;
ttttttt) a deletion at a residue corresponding to position 280 in SEQ ID NO: 1;
uuuuuuu) a deletion, V, M, L, I at a residue corresponding to position 281 in SEQ ID NO: 1;
vvvvvvv) a deletion, Q at a residue corresponding to position 282 in SEQ ID NO: 1;
wwwwwww) P, C at a residue corresponding to position 286 in SEQ ID NO: 1;
xxxxxxx) T, TVFFA (SEQ ID NO: 51), TSFFA (SEQ ID NO: 52), TFFFA (SEQ ID NO: 53), TMFFA (SEQ ID NO: 54), TVFHA (SEQ ID NO: 55) at a residue corresponding to position 287 in SEQ ID NO: 1;
yyyyyyy) H, F, M, Y, R, I, V, T, C, a deletion at a residue corresponding to position 288 in SEQ ID NO: 1;
zzzzzzz) a deletion, M, V, A, L, F, I at a residue corresponding to position 289 in SEQ ID NO: 1;
aaaaaaaa) a deletion, A, W, H, C, F, V, P, L at a residue corresponding to position 290 in SEQ ID NO: 1;
bbbbbbbb) a deletion, A, M, G, V at a residue corresponding to position 291 in SEQ ID NO: 1;
cccccccc) A, T at a residue corresponding to position 294 in SEQ ID NO: 1;
dddddddd) L, a deletion at a residue corresponding to position 295 in SEQ ID NO: 1;
eeeeeeee) M, L at a residue corresponding to position 296 in SEQ ID NO: 1;
ffffffff) M, Q, I, A, L, T at a residue corresponding to position 297 in SEQ ID NO: 1;
gggggggg) M, A, F, R, IM at a residue corresponding to position 298 in SEQ ID NO: 1;
hhhhhhhh) M, F at a residue corresponding to position 299 in SEQ ID NO: 1;
iiiiiiii) A at a residue corresponding to position 300 in SEQ ID NO: 1;
jjjjjjjj) M, R, E at a residue corresponding to position 304 in SEQ ID NO: 1;
kkkkkkkk) D at a residue corresponding to position 306 in SEQ ID NO: 1;
llllllll) L at a residue corresponding to position 309 in SEQ ID NO: 1;
mmmmmmmm) L, D at a residue corresponding to position 310 in SEQ ID NO: 1;
nnnnnnnn) L at a residue corresponding to position 311 in SEQ ID NO: 1;
oooooooo) S, SHVRSV (SEQ ID NO: 56), SHVRTV (SEQ ID NO: 57) at a residue corresponding to position 314 in SEQ ID NO: 1;
pppppppp) S, a deletion at a residue corresponding to position 315 in SEQ ID NO: 1;
qqqqqqqq) a deletion, V, L, I at a residue corresponding to position 316 in SEQ ID NO: 1;
rrrrrrrr) a deletion, H, N, Q, T at a residue corresponding to position 317 in SEQ ID NO: 1;
ssssssss) a deletion, Y, M, T, S, C, Q, R, H, V, W, I, L at a residue corresponding to position 318 in SEQ ID NO: 1;
tttttttt) a deletion, C, F, V, L, M at a residue corresponding to position 319 in SEQ ID NO: 1;
uuuuuuuu) F, I, V, Y, T, M, W at a residue corresponding to position 320 in SEQ ID NO: 1;
vvvvvvvv) C at a residue corresponding to position 321 in SEQ ID NO: 1;
wwwwwwww) G at a residue corresponding to position 323 in SEQ ID NO: 1;
xxxxxxxx) S, M at a residue corresponding to position 324 in SEQ ID NO: 1;
yyyyyyyy) V, I, C at a residue corresponding to position 326 in SEQ ID NO: 1;
zzzzzzzz) S at a residue corresponding to position 327 in SEQ ID NO: 1;
aaaaaaaaa) H, V, R, S, RELE (SEQ ID NO: 58) at a residue corresponding to position 328 in SEQ ID NO: 1;
bbbbbbbbb) E, a deletion, ELE at a residue corresponding to position 329 in SEQ ID NO: 1;
ccccccccc) a deletion, L, T, I at a residue corresponding to position 330 in SEQ ID NO: 1;
ddddddddd) a deletion, M, E, C, L, I at a residue corresponding to position 331 in SEQ ID NO: 1;
eeeeeeeee) A, W, C, M, I at a residue corresponding to position 334 in SEQ ID NO: 1;
fffffffff) Q, R at a residue corresponding to position 335 in SEQ ID NO: 1;
ggggggggg) K at a residue corresponding to position 336 in SEQ ID NO: 1;
hhhhhhhhh) M at a residue corresponding to position 338 in SEQ ID NO: 1;
iiiiiiiii) S at a residue corresponding to position 339 in SEQ ID NO: 1;
jjjjjjjjj) H, T at a residue corresponding to position 341 in SEQ ID NO: 1;
kkkkkkkkk) L, F at a residue corresponding to position 342 in SEQ ID NO: 1;
lllllllll) D, H, Y at a residue corresponding to position 348 in SEQ ID NO: 1;
mmmmmmmmm) V, C, T, F, A, W at a residue corresponding to position 351 in SEQ ID NO: 1;
nnnnnnnnn) E, K, D at a residue corresponding to position 352 in SEQ ID NO: 1;
ooooooooo) V, L, M at a residue corresponding to position 353 in SEQ ID NO: 1;
ppppppppp) C, F, L, Y, A, K, S, I at a residue corresponding to position 354 in SEQ ID NO: 1;
qqqqqqqqq) E at a residue corresponding to position 355 in SEQ ID NO: 1;
rrrrrrrrr) A at a residue corresponding to position 356 in SEQ ID NO: 1;
sssssssss) W at a residue corresponding to position 357 in SEQ ID NO: 1;
ttttttttt) M at a residue corresponding to position 359 in SEQ ID NO: 1;
uuuuuuuuu) S at a residue corresponding to position 360 in SEQ ID NO: 1;
vvvvvvvvv) C, S at a residue corresponding to position 362 in SEQ ID NO: 1;
wwwwwwwww) A, T, G at a residue corresponding to position 363 in SEQ ID NO: 1;
xxxxxxxxx) C, S at a residue corresponding to position 364 in SEQ ID NO: 1;
yyyyyyyyy) L, I, M, C at a residue corresponding to position 365 in SEQ ID NO: 1;
zzzzzzzzz) S, V, T at a residue corresponding to position 366 in SEQ ID NO: 1;
aaaaaaaaaa) A, C, S, H at a residue corresponding to position 367 in SEQ ID NO: 1;
bbbbbbbbbb) H at a residue corresponding to position 368 in SEQ ID NO: 1;
cccccccccc) H, Y, I, C, L, V, W, S, T, M at a residue corresponding to position 369 in SEQ ID NO: 1;
dddddddddd) Y, R, T, S, H, G, N, Q at a residue corresponding to position 370 in SEQ ID NO: 1;
eeeeeeeeee) W at a residue corresponding to position 371 in SEQ ID NO: 1;
ffffffffff) K, W, Q, H, S, R, N at a residue corresponding to position 372 in SEQ ID NO: 1;
gggggggggg) S, H, P, Q, E at a residue corresponding to position 373 in SEQ ID NO: 1;
hhhhhhhhhh) D, T, A, R, S, P, W, E, Q, H at a residue corresponding to position 374 in SEQ ID NO: 1;
iiiiiiiiii) P at a residue corresponding to position 375 in SEQ ID NO: 1;
jjjjjjjjjj) T, K, E, Q, H, D at a residue corresponding to position 376 in SEQ ID NO: 1;
kkkkkkkkkk) Y, R, N at a residue corresponding to position 377 in SEQ ID NO: 1;
llllllllll) S, Y, P, V, A at a residue corresponding to position 378 in SEQ ID NO: 1;
mmmmmmmmmm) G at a residue corresponding to position 379 in SEQ ID NO: 1;
nnnnnnnnnn) T at a residue corresponding to position 380 in SEQ ID NO: 1;
oooooooooo) V, S, I at a residue corresponding to position 381 in SEQ ID NO: 1;
pppppppppp) Q, H, T, Y, W, P at a residue corresponding to position 383 in SEQ ID NO: 1;
qqqqqqqqqq) I, P, A, M at a residue corresponding to position 384 in SEQ ID NO: 1;
rrrrrrrrrr) S, A at a residue corresponding to position 386 in SEQ ID NO: 1;
ssssssssss) G, S, H at a residue corresponding to position 387 in SEQ ID NO: 1;
tttttttttt) T, M, V, L at a residue corresponding to position 388 in SEQ ID NO: 1;
uuuuuuuuuu) R, K, E at a residue corresponding to position 389 in SEQ ID NO: 1;
vvvvvvvvvv) C, L, A at a residue corresponding to position 390 in SEQ ID NO: 1;
wwwwwwwwww) K at a residue corresponding to position 391 in SEQ ID NO: 1;
xxxxxxxxxx) D at a residue corresponding to position 394 in SEQ ID NO: 1;
yyyyyyyyyy) D, N at a residue corresponding to position 398 in SEQ ID NO: 1;
zzzzzzzzzz) V, L, I at a residue corresponding to position 402 in SEQ ID NO: 1;
aaaaaaaaaaa) L, V at a residue corresponding to position 406 in SEQ ID NO: 1;
bbbbbbbbbbb) D at a residue corresponding to position 407 in SEQ ID NO: 1;
ccccccccccc) N, Q, NTENAPR (SEQ ID NO: 59), NTENAPD (SEQ ID NO: 60) at a residue corresponding to position 410 in SEQ ID NO: 1;
ddddddddddd) T, TENAPD (SEQ ID NO: 61), TQNAPR (SEQ ID NO: 62), a deletion, TENAPR (SEQ ID NO: 63), TQNAPN (SEQ ID NO: 64) at a residue corresponding to position 411 in SEQ ID NO: 1;
eeeeeeeeeee) a deletion, Q at a residue corresponding to position 412 in SEQ ID NO: 1;
fffffffffff) a deletion, N, NNPN (SEQ ID NO: 65) at a residue corresponding to position 413 in SEQ ID NO: 1;
ggggggggggg) a deletion, A, S, N at a residue corresponding to position 414 in SEQ ID NO: 1;
hhhhhhhhhhh) a deletion, P at a residue corresponding to position 415 in SEQ ID NO: 1;
iiiiiiiiiii) a deletion, D, R, N, E at a residue corresponding to position 416 in SEQ ID NO: 1;
jjjjjjjjjjj) P at a residue corresponding to position 418 in SEQ ID NO: 1;
kkkkkkkkkkk) G at a residue corresponding to position 419 in SEQ ID NO: 1;
lllllllllll) E at a residue corresponding to position 420 in SEQ ID NO: 1;
mmmmmmmmmmm) M, V at a residue corresponding to position 424 in SEQ ID NO: 1;
nnnnnnnnnnn) V, L at a residue corresponding to position 425 in SEQ ID NO: 1;
ooooooooooo) M, L at a residue corresponding to position 426 in SEQ ID NO: 1;
ppppppppppp) T, N, S at a residue corresponding to position 430 in SEQ ID NO: 1;
qqqqqqqqqqq) I, L at a residue corresponding to position 431 in SEQ ID NO: 1;
rrrrrrrrrrr) T, F at a residue corresponding to position 432 in SEQ ID NO: 1;
sssssssssss) S, K at a residue corresponding to position 433 in SEQ ID NO: 1;
ttttttttttt) W at a residue corresponding to position 436 in SEQ ID NO: 1;
uuuuuuuuuuu) T at a residue corresponding to position 442 in SEQ ID NO: 1;
vvvvvvvvvvv) R, K, RASFA (SEQ ID NO: 66) at a residue corresponding to position 443 in SEQ ID NO: 1;
wwwwwwwwwww) a deletion at a residue corresponding to position 444 in SEQ ID NO: 1;
xxxxxxxxxxx) a deletion, M, T, S, A at a residue corresponding to position 445 in SEQ ID NO: 1;
yyyyyyyyyyy) a deletion at a residue corresponding to position 446 in SEQ ID NO: 1;
zzzzzzzzzzz) a deletion, A at a residue corresponding to position 447 in SEQ ID NO: 1;
aaaaaaaaaaaa) S, G at a residue corresponding to position 455 in SEQ ID NO: 1;
bbbbbbbbbbbb) G, E, D at a residue corresponding to position 459 in SEQ ID NO: 1;
cccccccccccc) D, G at a residue corresponding to position 461 in SEQ ID NO: 1;
dddddddddddd) R, G at a residue corresponding to position 462 in SEQ ID NO: 1;
eeeeeeeeeeee) L, Y at a residue corresponding to position 464 in SEQ ID NO: 1;
ffffffffffff) H at a residue corresponding to position 465 in SEQ ID NO: 1;
gggggggggggg) M at a residue corresponding to position 469 in SEQ ID NO: 1;
hhhhhhhhhhhh) C, G at a residue corresponding to position 470 in SEQ ID NO: 1;
iiiiiiiiiiii) Y, K, M, W, I, R at a residue corresponding to position 471 in SEQ ID NO: 1;
jjjjjjjjjjjj) I, F, C, Y, L, M at a residue corresponding to position 472 in SEQ ID NO: 1;
kkkkkkkkkkkk) E, EDGH (SEQ ID NO: 67), EDGY (SEQ ID NO: 68) at a residue corresponding to position 474 in SEQ ID NO: 1;
llllllllllll) D, a deletion at a residue corresponding to position 475 in SEQ ID NO: 1;
mmmmmmmmmmmm) a deletion at a residue corresponding to position 476 in SEQ ID NO: 1;
nnnnnnnnnnnn) a deletion, H, F, N, E, Y at a residue corresponding to position 477 in SEQ ID NO: 1;
oooooooooooo) W, I, M at a residue corresponding to position 478 in SEQ ID NO: 1;
pppppppppppp) T, H, Y, F, W at a residue corresponding to position 479 in SEQ ID NO: 1;
qqqqqqqqqqqq) V, L at a residue corresponding to position 480 in SEQ ID NO: 1;
rrrrrrrrrrrr) C, I, T at a residue corresponding to position 481 in SEQ ID NO: 1;
ssssssssssss) L at a residue corresponding to position 484 in SEQ ID NO: 1;
tttttttttttt) D at a residue corresponding to position 486 in SEQ ID NO: 1;
uuuuuuuuuuuu) I at a residue corresponding to position 489 in SEQ ID NO: 1;
vvvvvvvvvvvv) R, C, V at a residue corresponding to position 490 in SEQ ID NO: 1;
wwwwwwwwwwww) G, R, N at a residue corresponding to position 491 in SEQ ID NO: 1;
xxxxxxxxxxxx) F, H, Y, L at a residue corresponding to position 493 in SEQ ID NO: 1;
yyyyyyyyyyyy) N at a residue corresponding to position 494 in SEQ ID NO: 1;
zzzzzzzzzzzz) F, S, Y at a residue corresponding to position 496 in SEQ ID NO: 1;
aaaaaaaaaaaaa) L, V, I at a residue corresponding to position 503 in SEQ ID NO: 1;
bbbbbbbbbbbbb) R, RHPD (SEQ ID NO: 69) at a residue corresponding to position 506 in SEQ ID NO: 1;
ccccccccccccc) a deletion at a residue corresponding to position 507 in SEQ ID NO: 1;
ddddddddddddd) a deletion, P at a residue corresponding to position 508 in SEQ ID NO: 1;
eeeeeeeeeeeee) a deletion, D at a residue corresponding to position 509 in SEQ ID NO: 1;
fffffffffffff) A, V, C at a residue corresponding to position 513 in SEQ ID NO: 1;
ggggggggggggg) V at a residue corresponding to position 518 in SEQ ID NO: 1;
hhhhhhhhhhhhh) P at a residue corresponding to position 519 in SEQ ID NO: 1;
iiiiiiiiiiiii) E, P at a residue corresponding to position 521 in SEQ ID NO: 1;
jjjjjjjjjjjjj) Q, A, R, M, Y, D at a residue corresponding to position 523 in SEQ ID NO: 1;
kkkkkkkkkkkkk) S, A at a residue corresponding to position 524 in SEQ ID NO: 1;
lllllllllllll) S, T, D, G, E at a residue corresponding to position 525 in SEQ ID NO: 1;
mmmmmmmmmmmmm) P, T, V at a residue corresponding to position 527 in SEQ ID NO: 1;
nnnnnnnnnnnnn) A, V at a residue corresponding to position 528 in SEQ ID NO: 1;
ooooooooooooo) V, A at a residue corresponding to position 529 in SEQ ID NO: 1;
ppppppppppppp) L, V at a residue corresponding to position 539 in SEQ ID NO: 1;
qqqqqqqqqqqqq) T at a residue corresponding to position 540 in SEQ ID NO: 1;
rrrrrrrrrrrrr) E, D at a residue corresponding to position 542 in SEQ ID NO: 1;
sssssssssssss) A, K at a residue corresponding to position 546 in SEQ ID NO: 1;
ttttttttttttt) K, R at a residue corresponding to position 551 in SEQ ID NO: 1;
uuuuuuuuuuuuu) S at a residue corresponding to position 553 in SEQ ID NO: 1;
vvvvvvvvvvvvv) S, P, H at a residue corresponding to position 554 in SEQ ID NO: 1;
wwwwwwwwwwwww) F at a residue corresponding to position 555 in SEQ ID NO: 1;
xxxxxxxxxxxxx) K at a residue corresponding to position 557 in SEQ ID NO: 1;
yyyyyyyyyyyyy) P at a residue corresponding to position 559 in SEQ ID NO: 1;
zzzzzzzzzzzzz) G at a residue corresponding to position 560 in SEQ ID NO: 1;
aaaaaaaaaaaaaa) V, a deletion at a residue corresponding to position 562 in SEQ ID NO: 1;
bbbbbbbbbbbbbb) F at a residue corresponding to position 564 in SEQ ID NO: 1;
cccccccccccccc) V, F, FV at a residue corresponding to position 565 in SEQ ID NO: 1;
dddddddddddddd) E, VE at a residue corresponding to position 566 in SEQ ID NO: 1;
eeeeeeeeeeeeee) Q, E at a residue corresponding to position 583 in SEQ ID NO: 1;
ffffffffffffff) or any combination thereof.

13. The engineered ACL of claim 12, wherein the one or more amino acid alterations result in an engineered ACL comprising:
a) K, S at a residue corresponding to position 7 in SEQ ID NO: 1;
b) S at a residue corresponding to position 8 in SEQ ID NO: 1;
c) W, Y, F at a residue corresponding to position 10 in SEQ ID NO: 1;
d) E, P, D at a residue corresponding to position 12 in SEQ ID NO: 1;
e) I at a residue corresponding to position 15 in SEQ ID NO: 1;
f) E at a residue corresponding to position 16 in SEQ ID NO: 1;
g) F at a residue corresponding to position 19 in SEQ ID NO: 1;
h) R, K at a residue corresponding to position 52 in SEQ ID NO: 1;
i) L at a residue corresponding to position 54 in SEQ ID NO: 1;
j) A at a residue corresponding to position 64 in SEQ ID NO: 1;
k) R at a residue corresponding to position 66 in SEQ ID NO: 1;
l) A at a residue corresponding to position 67 in SEQ ID NO: 1;
m) L at a residue corresponding to position 73 in SEQ ID NO: 1;
n) K at a residue corresponding to position 74 in SEQ ID NO: 1;
o) T at a residue corresponding to position 82 in SEQ ID NO: 1;
p) I at a residue corresponding to position 87 in SEQ ID NO: 1;
q) L, M at a residue corresponding to position 90 in SEQ ID NO: 1;
r) T at a residue corresponding to position 94 in SEQ ID NO: 1;
s) L at a residue corresponding to position 100 in SEQ ID NO: 1;
t) V at a residue corresponding to position 111 in SEQ ID NO: 1;
u) N at a residue corresponding to position 115 in SEQ ID NO: 1;
v) S at a residue corresponding to position 117 in SEQ ID NO: 1;
w) T at a residue corresponding to position 119 in SEQ ID NO: 1;
x) D at a residue corresponding to position 121 in SEQ ID NO: 1;
y) I at a residue corresponding to position 129 in SEQ ID NO: 1;
z) G at a residue corresponding to position 131 in SEQ ID NO: 1;
aa) D, S at a residue corresponding to position 138 in SEQ ID NO: 1;
bb) E at a residue corresponding to position 139 in SEQ ID NO: 1;
cc) K, E, D at a residue corresponding to position 140 in SEQ ID NO: 1;
dd) T at a residue corresponding to position 144 in SEQ ID NO: 1;
ee) L, I at a residue corresponding to position 150 in SEQ ID NO: 1;
ff) S at a residue corresponding to position 165 in SEQ ID NO: 1;
gg) F at a residue corresponding to position 166 in SEQ ID NO: 1;
hh) L, A, D at a residue corresponding to position 172 in SEQ ID NO: 1;
ii) Q at a residue corresponding to position 182 in SEQ ID NO: 1;
jj) A at a residue corresponding to position 191 in SEQ ID NO: 1;
kk) A at a residue corresponding to position 199 in SEQ ID NO: 1;
ll) P at a residue corresponding to position 209 in SEQ ID NO: 1;
mm) C at a residue corresponding to position 212 in SEQ ID NO: 1;
nn) R at a residue corresponding to position 217 in SEQ ID NO: 1;
oo) T, A at a residue corresponding to position 221 in SEQ ID NO: 1;
pp) N, I, A, L, H at a residue corresponding to position 223 in SEQ ID NO: 1;
qq) Q at a residue corresponding to position 224 in SEQ ID NO: 1;
rr) E, T, A at a residue corresponding to position 227 in SEQ ID NO: 1;
ss) I, L, M at a residue corresponding to position 229 in SEQ ID NO: 1;
tt) Q, A at a residue corresponding to position 230 in SEQ ID NO: 1;
uu) H, Q, P at a residue corresponding to position 231 in SEQ ID NO: 1;
vv) Y, K, Q, N at a residue corresponding to position 232 in SEQ ID NO: 1;
ww) W, Y at a residue corresponding to position 233 in SEQ ID NO: 1;
xx) Y, F, H at a residue corresponding to position 234 in SEQ ID NO: 1;
yy) S at a residue corresponding to position 235 in SEQ ID NO: 1;
zz) D, H, T at a residue corresponding to position 238 in SEQ ID NO: 1;
aaa) T, H, C, S at a residue corresponding to position 240 in SEQ ID NO: 1;
bbb) C, M at a residue corresponding to position 243 in SEQ ID NO: 1;
ccc) M, V at a residue corresponding to position 249 in SEQ ID NO: 1;
ddd) H, M, Q at a residue corresponding to position 253 in SEQ ID NO: 1;
eee) M at a residue corresponding to position 254 in SEQ ID NO: 1;
fff) I, V at a residue corresponding to position 256 in SEQ ID NO: 1;
ggg) T, H, A, L, M at a residue corresponding to position 258 in SEQ ID NO: 1;
hhh) A at a residue corresponding to position 259 in SEQ ID NO: 1;
iii) K, C, N at a residue corresponding to position 262 in SEQ ID NO: 1;
jjj) F at a residue corresponding to position 268 in SEQ ID NO: 1;
kkk) R, Q at a residue corresponding to position 271 in SEQ ID NO: 1;
lll) E at a residue corresponding to position 275 in SEQ ID NO: 1;
mmm) L, M at a residue corresponding to position 277 in SEQ ID NO: 1;
nnn) V at a residue corresponding to position 281 in SEQ ID NO: 1;
ooo) V, I, F, T, H, Y at a residue corresponding to position 288 in SEQ ID NO: 1;
ppp) V, A at a residue corresponding to position 290 in SEQ ID NO: 1;
qqq) L, M at a residue corresponding to position 296 in SEQ ID NO: 1;
rrr) I at a residue corresponding to position 297 in SEQ ID NO: 1;
sss) A at a residue corresponding to position 300 in SEQ ID NO: 1;
ttt) E at a residue corresponding to position 304 in SEQ ID NO: 1;
uuu) D at a residue corresponding to position 306 in SEQ ID NO: 1;
vvv) L at a residue corresponding to position 316 in SEQ ID NO: 1;
www) S, R, H, W, Y, M, T, C, Q, V, I, L at a residue corresponding to position 318 in SEQ ID NO: 1;
xxx) M, F, T, Y, W at a residue corresponding to position 320 in SEQ ID NO: 1;
yyy) C at a residue corresponding to position 321 in SEQ ID NO: 1;
zzz) G at a residue corresponding to position 323 in SEQ ID NO: 1;
aaaa) V, H, R at a residue corresponding to position 328 in SEQ ID NO: 1;
bbbb) I, L at a residue corresponding to position 330 in SEQ ID NO: 1;
cccc) I at a residue corresponding to position 331 in SEQ ID NO: 1;
dddd) K at a residue corresponding to position 336 in SEQ ID NO: 1;
eeee) K at a residue corresponding to position 352 in SEQ ID NO: 1;
ffff) V, L at a residue corresponding to position 353 in SEQ ID NO: 1;
gggg) G at a residue corresponding to position 363 in SEQ ID NO: 1;
hhhh) S at a residue corresponding to position 364 in SEQ ID NO: 1;
iiii) C, L, I, M at a residue corresponding to position 365 in SEQ ID NO: 1;
jjjj) V at a residue corresponding to position 366 in SEQ ID NO: 1;
kkkk) H, S, T, M at a residue corresponding to position 369 in SEQ ID NO: 1;
llll) S, K, H, N at a residue corresponding to position 372 in SEQ ID NO: 1;
mmmm) Q, R, T, P, H, S, A, D, E at a residue corresponding to position 374 in SEQ ID NO: 1;
nnnn) T at a residue corresponding to position 376 in SEQ ID NO: 1;
oooo) A, P, S, V at a residue corresponding to position 378 in SEQ ID NO: 1;
pppp) T at a residue corresponding to position 388 in SEQ ID NO: 1;
qqqq) C, A at a residue corresponding to position 390 in SEQ ID NO: 1;
rrrr) D at a residue corresponding to position 394 in SEQ ID NO: 1;
ssss) N at a residue corresponding to position 398 in SEQ ID NO: 1;
tttt) L at a residue corresponding to position 406 in SEQ ID NO: 1;
uuuu) D at a residue corresponding to position 407 in SEQ ID NO: 1;
vvvv) N, E at a residue corresponding to position 416 in SEQ ID NO: 1;
wwww) F at a residue corresponding to position 432 in SEQ ID NO: 1;
xxxx) R, K at a residue corresponding to position 443 in SEQ ID NO: 1;
yyyy) A at a residue corresponding to position 445 in SEQ ID NO: 1;
zzzz) S, G at a residue corresponding to position 455 in SEQ ID NO: 1;
aaaaa) R at a residue corresponding to position 462 in SEQ ID NO: 1;
bbbbb) M at a residue corresponding to position 469 in SEQ ID NO: 1;
ccccc) G at a residue corresponding to position 470 in SEQ ID NO: 1;
ddddd) R, M, W at a residue corresponding to position 471 in SEQ ID NO: 1;
eeeee) V at a residue corresponding to position 480 in SEQ ID NO: 1;
fffff) I, C at a residue corresponding to position 481 in SEQ ID NO: 1;
ggggg) L at a residue corresponding to position 484 in SEQ ID NO: 1;
hhhhh) D at a residue corresponding to position 486 in SEQ ID NO: 1;
iiiii) C at a residue corresponding to position 490 in SEQ ID NO: 1;
jjjjj) R, G at a residue corresponding to position 491 in SEQ ID NO: 1;
kkkkk) L, H, Y at a residue corresponding to position 493 in SEQ ID NO: 1;
lllll) N at a residue corresponding to position 494 in SEQ ID NO: 1;
mmmmm) Y at a residue corresponding to position 496 in SEQ ID NO: 1;
nnnnn) P at a residue corresponding to position 508 in SEQ ID NO: 1;
ooooo) A, V, C at a residue corresponding to position 513 in SEQ ID NO: 1;
ppppp) V at a residue corresponding to position 518 in SEQ ID NO: 1;
qqqqq) P at a residue corresponding to position 519 in SEQ ID NO: 1;
rrrrr) E, P at a residue corresponding to position 521 in SEQ ID NO: 1;
sssss) R, Q, A, Y, M, D at a residue corresponding to position 523 in SEQ ID NO: 1;
ttttt) D, S, T, G, E at a residue corresponding to position 525 in SEQ ID NO: 1;
uuuuu) K at a residue corresponding to position 546 in SEQ ID NO: 1;
vvvvv) H, S, P at a residue corresponding to position 554 in SEQ ID NO: 1;
wwwww) F at a residue corresponding to position 555 in SEQ ID NO: 1;
xxxxx) G at a residue corresponding to position 560 in SEQ ID NO: 1;
yyyyy) V at a residue corresponding to position 565 in SEQ ID NO: 1;
zzzzz) Q, E at a residue corresponding to position 583 in SEQ ID NO: 1;
aaaaaa) or any combination thereof.

14. The engineered ACL of any one of claim 12 or 13, wherein the one or more amino acid alterations result in an engineered ACL comprising:
a) F at a residue corresponding to position 10 in SEQ ID NO: 1;
b) A at a residue corresponding to position 199 in SEQ ID NO: 1;
c) L, H, A at a residue corresponding to position 223 in SEQ ID NO: 1;
d) M at a residue corresponding to position 229 in SEQ ID NO: 1;
e) Q, A at a residue corresponding to position 230 in SEQ ID NO: 1;
f) P at a residue corresponding to position 231 in SEQ ID NO: 1;
g) N at a residue corresponding to position 232 in SEQ ID NO: 1;
h) W, Y at a residue corresponding to position 233 in SEQ ID NO: 1;
i) H at a residue corresponding to position 234 in SEQ ID NO: 1;
j) S at a residue corresponding to position 235 in SEQ ID NO: 1;
k) T at a residue corresponding to position 238 in SEQ ID NO: 1;
l) C, M at a residue corresponding to position 243 in SEQ ID NO: 1;
m) I, V at a residue corresponding to position 256 in SEQ ID NO: 1;
n) L, M at a residue corresponding to position 258 in SEQ ID NO: 1;
o) M at a residue corresponding to position 277 in SEQ ID NO: 1;
p) Y, H at a residue corresponding to position 288 in SEQ ID NO: 1;
q) A at a residue corresponding to position 300 in SEQ ID NO: 1;
r) V, T, C, I, L, Q, M, Y at a residue corresponding to position 318 in SEQ ID NO: 1;
s) W, Y at a residue corresponding to position 320 in SEQ ID NO: 1;
t) C at a residue corresponding to position 321 in SEQ ID NO: 1;
u) G at a residue corresponding to position 323 in SEQ ID NO: 1;
v) R at a residue corresponding to position 328 in SEQ ID NO: 1;
w) L at a residue corresponding to position 330 in SEQ ID NO: 1;
x) S at a residue corresponding to position 364 in SEQ ID NO: 1;
y) I, M at a residue corresponding to position 365 in SEQ ID NO: 1;
z) M at a residue corresponding to position 369 in SEQ ID NO: 1;
aa) N at a residue corresponding to position 372 in SEQ ID NO: 1;
bb) E at a residue corresponding to position 374 in SEQ ID NO: 1;
cc) V at a residue corresponding to position 378 in SEQ ID NO: 1;
dd) R at a residue corresponding to position 462 in SEQ ID NO: 1;
ee) M at a residue corresponding to position 469 in SEQ ID NO: 1;
ff) V at a residue corresponding to position 480 in SEQ ID NO: 1;
gg) C at a residue corresponding to position 481 in SEQ ID NO: 1;
hh) L at a residue corresponding to position 484 in SEQ ID NO: 1;
ii) C, A, V at a residue corresponding to position 513 in SEQ ID NO: 1;
jj) D at a residue corresponding to position 523 in SEQ ID NO: 1;
kk) G, E, T at a residue corresponding to position 525 in SEQ ID NO: 1;
ll) S, P at a residue corresponding to position 554 in SEQ ID NO: 1;
mm) G at a residue corresponding to position 560 in SEQ ID NO: 1;
nn) E at a residue corresponding to position 583 in SEQ ID NO: 1; or
oo) any combination thereof.

15. The engineered ACL of any one of claims 12 to 14, wherein the one or more amino acid alterations result in an engineered ACL comprising:
a) L, H at a residue corresponding to position 223 in SEQ ID NO: 1;
b) N at a residue corresponding to position 232 in SEQ ID NO: 1;
c) W, Y at a residue corresponding to position 233 in SEQ ID NO: 1;
d) H at a residue corresponding to position 234 in SEQ ID NO: 1;
e) T at a residue corresponding to position 238 in SEQ ID NO: 1;
f) L at a residue corresponding to position 258 in SEQ ID NO: 1;
g) Y, H at a residue corresponding to position 288 in SEQ ID NO: 1;
h) V, T, C, I, L at a residue corresponding to position 318 in SEQ ID NO: 1;
i) W, Y at a residue corresponding to position 320 in SEQ ID NO: 1;
j) C at a residue corresponding to position 321 in SEQ ID NO: 1;
k) G at a residue corresponding to position 323 in SEQ ID NO: 1;
l) S at a residue corresponding to position 364 in SEQ ID NO: 1;
m) C, A at a residue corresponding to position 513 in SEQ ID NO: 1;
n) D at a residue corresponding to position 523 in SEQ ID NO: 1; or
o) any combination thereof.

16. The engineered ACL of any one of claims 1 to 7, wherein the engineered ACL comprises one or more amino acid alterations selected from the group consisting of: I4M, N7D, N7K, N7Q, N7S, N7T, A8I, A8S, T9A, T9K, T9S, I10F, I10H, I10P, I10V, I10W, I10Y, N12D, N12E, N12H, N12P, I13C, I13M, V15D, V15I, V15L, V15P, D16E, D16H, D16N, Y19F, Y19L, Y19P, Y19Q, D20W, K22S, G30D, G31A, E35G, E35P, T36H, S39A, V40C, V40F, V40L, V40M, A43G, K44D, K44H, K44S, K44T, Q47R, Q47RTYTQKEL (“RTYTQKEL” disclosed as SEQ ID NO: 15), Q47RTYTQREL (“RTYTQREL” disclosed as SEQ ID NO: 16), Q47RTYT (“RTYT” disclosed as SEQ ID NO: 17), d48, d49, L49Y, d50, S50T, d51, Q51F, Q51H, Q51L, Q51R, Q51W, Q51Y, d52, A52K, A52R, d53, d54, M54L, W55L, K60R, Y61F, Y61L, L64A, L64G, S66R, S67A, Y69L, C72G, C72T, R73F, R73G, R73L, R73V, H74K, D76T, E78V, D80G, P81PG, S82A, S82E, S82G, S82N, S82R, S82T, d83, M83K, M83P, M83R, M83S, V86I, L87I, L87V, L87VAV, d88, I88A, d89, T89I, T89L, T89V, F90C, F90L, F90M, F90V, F90W, F91A, F91L, F91M, F91S, V94C, V94CIEAA (“CIEAA” disclosed as SEQ ID NO: 18), V94S, V94T, V94TIEAA (“TIEAA” disclosed as SEQ ID NO: 19), d95, d96, L96E, d97, d98, P98A, P98L, V99C, V99M, V100S, V100M, V100L, H101F, H101M, W102H, W102F, W102L, A103G, A104V, A104L, A104T, L105V, L105W, L105H, L105A, L105I, D106S, D106Y, D106R, D106K, L107A, L107C, L107V, A109L, A109V, A109Q, I111V, I111A, I111M, I111L, I111C, I111VTPAN (“VTPAN” disclosed as SEQ ID NO: 20), S112T, d112, d113, P113L, d114, G114A, G114L, G114V, d115, S115N, T116A, T116W, T116P, G117S, S119T, Q121R, Q121D, Q126H, F127M, F127L, F127MRHTG (“MRHTG” disclosed as SEQ ID NO: 21), F127MRITG (“MRITG” disclosed as SEQ ID NO: 22), d128, d129, V129I, V129H, d130, d131, T131G, T131D, V136L, Y137T, A138D, A138S, K139E, A140E, A140K, A140D, V144T, T149R, K150I, K150L, Y152A, Y152T, Y152C, Y152I, N153G, N153S, A159G, E162S, E162H, E162R, E162SIDAF (“SIDAF” disclosed as SEQ ID NO: 23), E162RIESF (“RIESF” disclosed as SEQ ID NO: 24), E162HFDSF (“HFDSF” disclosed as SEQ ID NO: 25), E162HFDAF (“HFDAF” disclosed as SEQ ID NO: 26), E162HIDSF (“HIDSF” disclosed as SEQ ID NO: 27), L163F, L163I, d163, d164, E164D, d165, A165S, d166, L166F, D167L, A170G, M172L, M172A, M172D, R182Q, H189E, H189G, S191A, R194D, I195T, I195P, I195M, A196T, A196I, Y197W, Y197C, L198Y, L198V, L198S, L198W, L198I, L198M, L198T, G199M, G199L, G199W, G199P, G199F, G199C, G199A, M200W, S201F, S201T, S201N, S201P, S201Y, S201L, S202T, S202H, S202A, S202Q, S202E, S202L, S202G, S205T, V208F, S209P, A211G, V212A, V212C, R213M, R213V, R213E, L214V, T215S, S217Q, S217R, S217Y, S217H, S217K, S217G, T220W, T220M, T220V, S221T, S221A, C222S, C222N, S223A, S223N, S223I, S223L, S223H, E224T, E224Q, E224H, E224I, E224N, E224M, I225L, I225A, I225F, I225Q, S226Y, S226C, S226T, S226W, N227G, N227E, N227M, N227T, N227A, N227GKWSQD (“GKWSQD” disclosed as SEQ ID NO: 28), N227GKWSQSY (“GKWSQSY” disclosed as SEQ ID NO: 29), N227GKWSQ (“GKWSQ” disclosed as SEQ ID NO: 30), N227GKWSQDLK (“GKWSQDLK” disclosed as SEQ ID NO: 31), N227GKWSQQLQ (“GKWSQQLQ” disclosed as SEQ ID NO: 32), d228, d229, A229F, A229S, A229L, A229G, A229W, A229I, A229M, A229H, d230, S230R, S230M, S230P, S230T, S230E, S230Q, S230A, d231, E231Q, E231P, E231H, E231QDLQ (“QDLQ” disclosed as SEQ ID NO: 33), d232, S232W, S232A, S232Y, S232N, S232K, S232Q, S232D, d233, L233W, L233R, L233M, L233Y, L233P, d234, A234F, A234Y, A234T, A234K, A234R, A234Q, A234P, A234G, A234H, A234E, T235H, T235S, T235R, T235K, T235M, T235Q, T235D, T235G, Q238P, Q238N, Q238H, Q238T, Q238D, Q238DISAC (“DISAC” disclosed as SEQ ID NO: 34), Q238DVS, I239V, I239F, I239R, d239, d240, A240F, A240H, A240T, A240W, A240S, A240L, A240C, A240V, d241, A241C, A241M, A241P, A241I, d242, A242G, A242V, A242C, I243L, I243C, I243M, I244M, I244V, V246W, V246F, V246L, V246Y, S247F, S247C, H248S, H248P, L249A, L249V, L249M, F250M, F250A, F250W, G251A, G251H, L252I, L252M, L252W, L252Q, S253T, S253Q, S253N, S253I, S253H, S253V, S253M, K254E, K254I, K254T, K254Y, K254H, K254V, K254L, K254P, K254A, K254M, K254F, K254S, K254G, F255M, F255T, F255I, F255N, F255Y, F255C, F255ILLANV (“ILLANV” disclosed as SEQ ID NO: 35), F255ILLANVHY (“ILLANVHY” disclosed as SEQ ID NO: 36), F255ILLLNVHY (“ILLLNVHY” disclosed as SEQ ID NO: 37), F255ILLLNV (“ILLLNV” disclosed as SEQ ID NO: 38), F255ILLLNVHK (“ILLLNVHK” disclosed as SEQ ID NO: 39), F255ILLHNVHK (“ILLHNVHK” disclosed as SEQ ID NO: 40), F255ILLSNVHY (“ILLSNVHY” disclosed as SEQ ID NO: 41), L256I, L256V, L256H, L256C, L256F, d256, d257, I257L, I257T, I257N, d258, G258S, G258L, G258P, G258M, G258H, G258T, G258A, d259, N259C, N259A, d260, P260F, P260L, P260M, P260V, d261, H261Y, H261M, H261W, H261A, H261F, H261L, H261S, d262, A262M, A262K, A262Y, A262R, A262G, A262C, A262L, A262V, A262S, A262N, A264G, A264W, A264I, A264S, A264V, T265R, T265C, T265H, T265K, T266I, T266V, T266Q, T266C, V267Y, Y268V, Y268M, Y268T, Y268F, Y268IMPR (“IMPR” disclosed as SEQ ID NO: 42), Y268EIMNR (“IMNR” disclosed as SEQ ID NO: 43), H269M, d269, d270, N270P, d271, G271R, G271Q, F272W, L274P, L274PEEMLRAIQ (“PEEMLRAIQ” disclosed as SEQ ID NO: 44), L274PEEMLQAIQ (“PEEMLQAIQ” disclosed as SEQ ID NO: 45), 1275D, I275A, 1275E, I275R, I275K, I275EEMLQAIQ (“EEMLQAIQ” disclosed as SEQ ID NO: 46), I275EEMLRAIQ (“EEMLRAIQ” disclosed as SEQ ID NO: 47), d275-282, I275KEMLQAIQ (“KEMLQAIQ” disclosed as SEQ ID NO: 48), d276, d277, V277M, V277Y, V277L, V277F, d278, d279, E279Q, E279R, E279QAIQ (“QAIQ” disclosed as SEQ ID NO: 49), E279RAIQ (“RAIQ” disclosed as SEQ ID NO: 50), d280, d281, Q281V, Q281M, Q281L, Q281I, d282, K282Q, V286P, V286C, N287T, N287TVFFA (“TVFFA” disclosed as SEQ ID NO: 51), N287TSFFA (“TSFFA” disclosed as SEQ ID NO: 52), N287TFFFA (“TFFFA” disclosed as SEQ ID NO: 53), N287TMFFA (“TMFFA” disclosed as SEQ ID NO: 54), N287TVFHA (“TVFHA” disclosed as SEQ ID NO: 55), S288H, S288F, S288M, S288Y, S288R, S288I, S288V, S288T, S288C, d288, d289, W289M, W289V, W289A, W289L, W289F, W289I, d290, T290A, T290W, T290H, T290C, T290F, T290V, T290P, T290L, d291, L291A, L291M, L291G, L291V, P294A, P294T, I295L, d295, I296M, I296L, V297M, V297Q, V297I, V297A, V297L, V297T, L298M, L298A, L298F, L298R, L298Im, L299M, L299F, T300A, 1304M, 1304R, 1304E, E306D, 1309L, P310L, P310D, S311L, A314S, A314SHVRSV (“SHVRSV” disclosed as SEQ ID NO: 56), A314SHVRTV (“SHVRTV” disclosed as SEQ ID NO: 57), H315S, d315, d316, M316V, M316L, M316I, d317, R317H, R317N, R317Q, R317T, d318, A318Y, A318M, A318T, A318S, A318C, A318Q, A318R, A318H, A318V, A318W, A318I, A318L, d319, I319C, I319F, I319V, I319L, I319M, L320F, L320I, L320V, L320Y, L320T, L320M, L320W, S321C, A323G, A324S, A324M, L326V, L326I, L326C, G327S, A328H, A328V, A328R, A328S, A328RELE (“RELE” disclosed as SEQ ID NO: 58), N329E, d329, N329ELE, d330, V330L, V330T, V330I, d331, T331M, T331E, T331C, T331L, T331I, L334A, L334W, L334C, L334M, L334I, L335Q, L335R, T336K, V338M, T339S, N341H, N341T, K342L, K342F, E348D, E348H, E348Y, L351V, L351C, L351T, L351F, L351A, L351W, R352E, R352K, R352D, I353V, I353L, I353M, V354C, V354F, V354L, V354Y, V354A, V354K, V354S, V354I, Q355E, G356A, Y357W, L359M, T360S, T362C, T362S, S363A, S363T, S363G, P364C, P364S, V365L, V365I, V365M, V365C, A366S, A366V, A366T, T367A, T367C, T367S, T367H, L368H, F369H, F369Y, F369I, F369C, F369L, F369V, F369W, F369S, F369T, F369M, D370Y, D370R, D370T, D370S, D370H, D370G, D370N, D370Q, P371W, E372K, E372W, E372Q, E372H, E372S, E372R, E372N, D373S, D373H, D373P, D373Q, D373E, K374D, K374T, K374A, K374R, K374S, K374P, K374W, K374E, K374Q, K374H, E375P, R376T, R376K, R376E, R376Q, R376H, R376D, H377Y, H377R, H377N, I378S, I378Y, I378P, I378V, I378A, R379G, S380T, C381V, C381S, C381I, K383Q, K383H, K383T, K383Y, K383W, K383P, L384I, L384P, L384A, L384M, P386S, P386A, N387G, N387S, N387H, S388T, S388M, S388V, S388L, Q389R, Q389K, Q389E, V390C, V390L, V390A, R391K, N394D, V398D, V398N, A402V, A402L, A402I, D406L, D406V, P407D, L410N, L410Q, L410NTENAPR (“NTENAPR” disclosed as SEQ ID NO: 59), L410NTENAPD (“NTENAPD” disclosed as SEQ ID NO: 60), D411T, D411TENAPD (“TENAPD” disclosed as SEQ ID NO: 61), D411TQNAPR (“TQNAPR” disclosed as SEQ ID NO: 62), d411, D411TENAPR (“TENAPR” disclosed as SEQ ID NO: 63), D411TQNAPN (“TQNAPN” disclosed as SEQ ID NO: 64), d412, E412Q, d413, A413N, A413NNPN (“NNPN” disclosed as SEQ ID NO: 65), d414, I414A, I414S, I414N, d415, K415P, d416, Q416D, Q416R, Q416N, Q416E, T418P, L419G, P420E, L424M, L424V, W425V, W425L, I426M, I426L, Q430T, Q430N, Q430S, V431I, V431L, M432T, M432F, D433S, D433K, H436W, N442T, E443R, E443K, E443RASFA (“RASFA” disclosed as SEQ ID NO: 66), d444, d445, C445M, C445T, C445S, C445A, d446, d447, V447A, D455S, D455G, Y459G, Y459E, Y459D, R461D, R461G, H462R, H462G, F464L, F464Y, R465H, V469M, A470C, A470G, L471Y, L471K, L471M, L471W, L471I, L471R, V472I, V472F, V472C, V472Y, V472L, V472M, K474E, K474EDGH (“EDGH” disclosed as SEQ ID NO: 67), K474EDGY (“EDGY” disclosed as SEQ ID NO: 68), Q475D, d475, d476, d477, R477H, R477F, R477N, R477E, R477Y, Y478W, Y478I, Y478M, M479T, M479H, M479Y, M479F, M479W, I480V, I480L, V481C, V481I, V481T, T484L, E486D, K489I, S490R, S490C, S490V, Q491G, Q491R, Q491N, K493F, K493H, K493Y, K493L, Q494N, A496F, A496S, A496Y, M503L, M503V, M503I, G506R, G506RHPD (“RHPD” disclosed as SEQ ID NO: 69), d507, d508, A508P, d509, Q509D, T513A, T513V, T513C, I518V, Q519P, V521E, V521P, K523Q, K523A, K523R, K523M, K523Y, K523D, G524S, G524A, N525S, N525T, N525D, N525G, N525E, A527P, A527T, A527V, P528A, P528V, R529V, R529A, Y539L, Y539V, D540T, V542E, V542D, T546A, T546K, Q551K, Q551R, P553S, K554S, K554P, K554H, Y555F, Q557K, H559P, A560G, I562V, d562, V564F, I565V, I565F, I565FV, D566E, D566VE, A583Q, A583E, and any combination thereof.

17. The engineered ACL claim 16, wherein the engineered ACL comprises one or more amino acid alterations selected from the group consisting of: N7K, N7S, A8S, I10W, I10Y, I10F, N12E, N12P, N12D, V15I, D16E, Y19F, A52R, A52K, M54L, L64A, S66R, S67A, R73L, H74K, S82T, L87I, F90L, F90M, V94T, V100L, Ily 1V, S115N, G117S, S119T, Q121D, V129I, T131G, A138D, A138S, K139E, A140K, A140E, A140D, V144T, K150L, K150I, A165S, L166F, M172L, M172A, M172D, R182Q, S191A, G199A, S209P, V212C, S217R, S221T, S221A, S223N, S223I, S223A, S223L, S223H, E224Q, N227E, N227T, N227A, A229I, A229L, A229M, S230Q, S230A, E231H, E231Q, E231P, S232Y, S232K, S232Q, S232N, L233W, L233Y, A234Y, A234F, A234H, T235S, Q238D, Q238H, Q238T, A240T, A240H, A240C, A240S, I243C, I243M, L249M, L249V, S253H, S253M, S253Q, K254M, L256I, L256V, G258T, G258H, G258A, G258L, G258M, N259A, A262K, A262C, A262N, Y268F, G271R, G271Q, I275E, V277L, V277M, Q281V, S288V, S288I, S288F, S288T, S288H, S288Y, T290V, T290A, I296L, I296M, V297I, T300A, I304E, E306D, M316L, A318S, A318R, A318H, A318W, A318Y, A318M, A318T, A318C, A318Q, A318V, A318I, A318L, L320M, L320F, L320T, L320Y, L320W, S321C, A323G, A328V, A328H, A328R, V330I, V330L, T331I, T336K, R352K, I353V, I353L, S363G, P364S, V365C, V365L, V365I, V365M, A366V, F369H, F369S, F369T, F369M, E372S, E372K, E372H, E372N, K374Q, K374R, K374T, K374P, K374H, K374S, K374A, K374D, K374E, R376T, I378A, I378P, I378S, I378V, S388T, V390C, V390A, N394D, V398N, D406L, P407D, Q416N, Q416E, M432F, E443R, E443K, C445A, D455S, D455G, H462R, V469M, A470G, L471R, L471M, L471W, I480V, V481I, V481C, T484L, E486D, S490C, Q491R, Q491G, K493L, K493H, K493Y, Q494N, A496Y, A508P, T513A, T513V, T513C, I518V, Q519P, V521E, V521P, K523R, K523Q, K523A, K523Y, K523M, K523D, N525D, N525S, N525T, N525G, N525E, T546K, K554H, K554S, K554P, Y555F, A560G, I565V, A583Q, A583E and any combination thereof.

18. The engineered ACL of claim 16 or claim 17, wherein the engineered ACL comprises one or more amino acid alterations selected from the group consisting of: I10F, G199A, S223L, S223H, S223A, A229M, S230Q, S230A, E231P, S232N, L233W, L233Y, A234H, T235S, Q238T, I243C, I243M, L256I, L256V, G258L, G258M, V277M, S288Y, S288H, T300A, A318V, A318T, A318C, A318I, A318L, A318Q, A318M, A318Y, L320W, L320Y, S321C, A323G, A328R, V330L, P364S, V365I, V365M, F369M, E372N, K374E, I378V, H462R, V469M, I480V, V481C, T484L, T513C, T513A, T513V, K523D, N525G, N525E, N525T, K554S, K554P, A560G, A583E and any combination thereof.

19. The engineered ACL of any one of claims 16 to 18, wherein the engineered ACL comprises one or more amino acid alterations selected from the group consisting of: L233W, P364S, S232N, L320W, A318V, S321C, S223L, L320Y, T513C, Q238T, L233Y, K523D, A318T, S288Y, A318C, A234H, A323G, T513A, S288H, S223H, A318I, A318L, G258L and any combination thereof.

20. The engineered ACL of any one of claims 1 to 19, wherein the engineered ACL comprises a single alteration or a combination of alterations described in TABLE 8.

21. The engineered ACL of any one of claims 1 to 7, wherein the ACL comprises one or more amino acid alterations at a position corresponding to position 5, 6, 7, 8, 10, 12, 13, 14, 15, 16, 19, 20, 22, 25, 28, 37, 39, 40, 41, 43, 44, 45, 48, 50, 51, 53, 57, 58, 59, 61, 62, 63, 66, 67, 69, 71, 72, 74, 75, 76, 77, 78, 80, 81, 83, 84, 85, 86, 87, 88, 92, 93, 94, 95, 97, 98, 99, 100, 101, 103, 104, 106, 107, 108, 109, 110, 113, 114, 115, 116, 119, 120, 121, 122, 123, 124, 125, 126, 127, 128, 130, 131, 136, 137, 140, 141, 142, 145, 147, 148, 149, 150, 151, 157, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 216, 218, 219, 220, 221, 222, 224, 225, 226, 227, 228, 230, 231, 232, 233, 234, 235, 236, 237, 238, 239, 240, 241, 242, 243, 244, 245, 246, 247, 251, 252, 253, 254, 255, 256, 257, 259, 260, 262, 263, 264, 265, 266, 267, 268, 269, 270, 271, 272, 273, 274, 275, 277, 278, 279, 281, 282, 283, 284, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 341, 342, 343, 344, 345, 346, 347, 348, 349, 351, 352, 353, 354, 355, 357, 358, 359, 361, 362, 363, 365, 366, 367, 368, 369, 370, 372, 373, 375, 376, 377, 378, 379, 380, 381, 382, 383, 384, 385, 386, 387, 388, 389, 391, 392, 394, 395, 396, 398, 399, 400, 401, 402, 404, 405, 406, 407, 409, 411, 413, 414, 415, 416, 417, 418, 419, 420, 421, 425, 430, 432, 433, 434, 435, 441, 442, 443, 446, 447, 449, 450, 451, 452, 453, 455, 456, 458, 459, 460, 461, 462, 463, 464, 465, 466, 467, 468, 469, 470, 471, 473, 474, 476, 478, 479, 480, 481, 483, 485, 490, 492, 493, 496, 497, 498, 502, 504, 507, 508, 510, 511, 512, 513, 514, 515, 516, 518, 519, 521, 526, 527, 532, 535, 540, 542, 546, 552, 554, 558, 560, 561, 563, 564, 565, 566, 567, 568, 569, 570, 572, 573, 574, 575, 577, 578, 583, 584, 585, 587, 588, 591, 593, 595, 596, 598, 599, 601, or a combination thereof, in SEQ ID NO: 2.

22. The engineered ACL of claim 21, wherein the ACL comprises one or more amino acid alterations at a position corresponding to position 5, 6, 7, 8, 10, 12, 13, 14, 15, 16, 19, 20, 22, 25, 28, 37, 39, 40, 41, 43, 45, 50, 51, 53, 57, 58, 59, 61, 62, 63, 67, 69, 71, 72, 74, 75, 76, 77, 78, 80, 81, 83, 84, 85, 86, 87, 88, 92, 93, 94, 95, 97, 98, 99, 100, 101, 103, 104, 106, 108, 109, 110, 113, 116, 119, 120, 121, 123, 124, 125, 126, 127, 128, 130, 131, 136, 137, 140, 141, 142, 145, 147, 148, 149, 150, 151, 159, 161, 163, 164, 165, 167, 168, 169, 170, 173, 175, 176, 177, 181, 183, 185, 186, 187, 189, 190, 191, 192, 193, 194, 195, 196, 201, 205, 208, 209, 211, 212, 213, 214, 215, 218, 219, 220, 221, 222, 224, 226, 227, 228, 230, 232, 233, 234, 235, 236, 237, 238, 239, 240, 241, 242, 243, 244, 245, 246, 247, 252, 253, 254, 255, 256, 260, 262, 263, 264, 265, 266, 267, 268, 270, 271, 272, 273, 274, 275, 277, 278, 279, 281, 283, 285, 286, 287, 290, 291, 292, 294, 295, 296, 298, 299, 300, 302, 303, 304, 305, 306, 307, 308, 311, 312, 313, 314, 315, 316, 318, 321, 322, 330, 331, 333, 334, 335, 336, 337, 338, 339, 340, 342, 343, 344, 345, 346, 347, 348, 349, 351, 352, 353, 354, 355, 357, 358, 359, 362, 363, 365, 366, 367, 368, 369, 370, 375, 376, 377, 378, 379, 380, 381, 382, 383, 384, 385, 386, 387, 388, 389, 391, 392, 394, 396, 398, 399, 400, 401, 404, 406, 407, 409, 411, 414, 416, 419, 420, 421, 425, 430, 432, 433, 434, 435, 441, 442, 443, 446, 447, 450, 451, 452, 453, 456, 460, 461, 462, 463, 465, 466, 467, 468, 469, 470, 471, 473, 476, 478, 479, 480, 481, 485, 490, 492, 498, 507, 508, 510, 511, 512, 514, 515, 516, 518, 519, 521, 526, 527, 532, 535, 540, 542, 546, 552, 554, 560, 561, 563, 564, 565, 566, 567, 568, 569, 570, 572, 573, 574, 575, 577, 578, 583, 584, 585, 587, 588, 591, 593, 595, 596, 598, 599, 601 or a combination thereof, in SEQ ID NO: 2.

23. The engineered ACL of claim 21 or 22 wherein the ACL comprises one or more amino acid alterations at a position corresponding to 5, 6, 7, 8, 10, 12, 14, 15, 16, 19, 20, 22, 28, 39, 41, 43, 45, 50, 57, 58, 61, 62, 63, 67, 69, 71, 72, 76, 77, 78, 81, 84, 85, 86, 87, 92, 93, 94, 95, 98, 104, 108, 109, 113, 116, 119, 120, 123, 124, 125, 126, 127, 130, 131, 136, 140, 141, 142, 145, 147, 148, 149, 151, 159, 161, 163, 164, 167, 168, 169, 170, 173, 175, 176, 177, 181, 183, 185, 186, 187, 190, 191, 193, 194, 195, 201, 205, 209, 214, 218, 219, 220, 221, 226, 228, 230, 232, 234, 237, 238, 239, 241, 242, 243, 244, 246, 247, 253, 254, 260, 262, 263, 266, 267, 272, 273, 274, 278, 279, 281, 283, 285, 290, 291, 292, 295, 296, 298, 300, 302, 303, 304, 305, 307, 311, 313, 314, 315, 316, 318, 321, 322, 330, 333, 334, 336, 337, 338, 339, 340, 344, 345, 346, 347, 348, 349, 351, 352, 353, 354, 355, 357, 358, 359, 362, 363, 365, 367, 369, 375, 376, 379, 382, 383, 384, 386, 387, 388, 389, 399, 400, 401, 404, 407, 409, 416, 419, 420, 421, 430, 432, 433, 434, 441, 442, 446, 450, 452, 453, 456, 460, 461, 465, 466, 471, 473, 476, 479, 480, 481, 485, 490, 492, 507, 511, 514, 515, 516, 518, 519, 521, 526, 527, 532, 540, 542, 546, 554, 561, 564, 565, 566, 567, 568, 570, 572, 574, 575, 577, 584, 587, 595, 596, 599 or a combination thereof, in SEQ ID NO: 2.

24. The engineered ACL of any one of claims 21 to 23 wherein the ACL comprises one or more amino acid alterations at a position corresponding to 5, 6, 7, 8, 10, 12, 14, 15, 19, 20, 22, 41, 45, 50, 58, 62, 63, 67, 69, 72, 76, 77, 81, 86, 87, 92, 93, 94, 95, 98, 104, 108, 113, 116, 120, 123, 124, 125, 126, 127, 130, 136, 140, 141, 142, 145, 147, 148, 149, 151, 159, 161, 163, 164, 167, 168, 169, 175, 176, 177, 185, 186, 187, 190, 191, 193, 195, 201, 205, 209, 214, 219, 220, 221, 228, 230, 232, 234, 237, 238, 239, 241, 242, 243, 244, 246, 247, 253, 254, 260, 263, 266, 267, 272, 273, 274, 278, 279, 281, 283, 291, 292, 295, 298, 300, 302, 303, 304, 305, 307, 311, 313, 314, 315, 318, 321, 322, 330, 333, 334, 336, 337, 338, 340, 344, 348, 349, 351, 353, 354, 355, 357, 358, 359, 362, 363, 365, 367, 369, 376, 379, 382, 383, 386, 387, 388, 389, 399, 400, 401, 404, 407, 409, 420, 421, 430, 432, 433, 434, 442, 446, 450, 452, 453, 456, 460, 461, 465, 466, 473, 476, 479, 480, 481, 485, 492, 507, 514, 515, 516, 518, 519, 521, 526, 527, 532, 540, 542, 554, 564, 565, 567, 568, 570, 572, 575, 584, 587, 595, 596 or a combination thereof, in SEQ ID NO: 2.

25. The engineered ACL of any one of claims 1 to 7, wherein the one or more amino acid alterations result in an engineered ACL comprising:
a) R, E at a residue corresponding to position 5 in SEQ ID NO: 2;
b) L at a residue corresponding to position 6 in SEQ ID NO: 2;
c) K, T at a residue corresponding to position 7 in SEQ ID NO: 2;
d) P at a residue corresponding to position 8 in SEQ ID NO: 2;
e) K, D, P, N, R at a residue corresponding to position 10 in SEQ ID NO: 2;
f) A, I, V, M at a residue corresponding to position 12 in SEQ ID NO: 2;
g) C at a residue corresponding to position 13 in SEQ ID NO: 2;
h) P, A, Q at a residue corresponding to position 14 in SEQ ID NO: 2;
i) E, L, S, R at a residue corresponding to position 15 in SEQ ID NO: 2;
j) C, L, I at a residue corresponding to position 16 in SEQ ID NO: 2;
k) D, R at a residue corresponding to position 19 in SEQ ID NO: 2;
l) H, K, R, D at a residue corresponding to position 20 in SEQ ID NO: 2;
m) D, E at a residue corresponding to position 22 in SEQ ID NO: 2;
n) N at a residue corresponding to position 25 in SEQ ID NO: 2;
o) M at a residue corresponding to position 28 in SEQ ID NO: 2;
p) C at a residue corresponding to position 37 in SEQ ID NO: 2;
q) R at a residue corresponding to position 39 in SEQ ID NO: 2;
r) T at a residue corresponding to position 40 in SEQ ID NO: 2;
s) M, L, V, I at a residue corresponding to position 41 in SEQ ID NO: 2;
t) S at a residue corresponding to position 43 in SEQ ID NO: 2;
u) M at a residue corresponding to position 44 in SEQ ID NO: 2;
v) I, V at a residue corresponding to position 45 in SEQ ID NO: 2;
w) P at a residue corresponding to position 48 in SEQ ID NO: 2;
x) I, F, R, V, L at a residue corresponding to position 50 in SEQ ID NO: 2;
y) R at a residue corresponding to position 51 in SEQ ID NO: 2;
z) L at a residue corresponding to position 53 in SEQ ID NO: 2;
aa) F at a residue corresponding to position 57 in SEQ ID NO: 2;
bb) V, L, M, F at a residue corresponding to position 58 in SEQ ID NO: 2;
cc) V at a residue corresponding to position 59 in SEQ ID NO: 2;
dd) P, E at a residue corresponding to position 61 in SEQ ID NO: 2;
ee) D at a residue corresponding to position 62 in SEQ ID NO: 2;
ff) D at a residue corresponding to position 63 in SEQ ID NO: 2;
gg) G at a residue corresponding to position 66 in SEQ ID NO: 2;
hh) K at a residue corresponding to position 67 in SEQ ID NO: 2;
ii) F, Y, L at a residue corresponding to position 69 in SEQ ID NO: 2;
jj) F, W at a residue corresponding to position 71 in SEQ ID NO: 2;
kk) A, R at a residue corresponding to position 72 in SEQ ID NO: 2;
ll) F at a residue corresponding to position 74 in SEQ ID NO: 2;
mm) D at a residue corresponding to position 75 in SEQ ID NO: 2;
nn) T, D, A, R at a residue corresponding to position 76 in SEQ ID NO: 2;
oo) R at a residue corresponding to position 77 in SEQ ID NO: 2;
pp) A at a residue corresponding to position 78 in SEQ ID NO: 2;
qq) K at a residue corresponding to position 80 in SEQ ID NO: 2;
rr) C at a residue corresponding to position 81 in SEQ ID NO: 2;
ss) S, H, Q, N at a residue corresponding to position 83 in SEQ ID NO: 2;
tt) Y, H at a residue corresponding to position 84 in SEQ ID NO: 2;
uu) F at a residue corresponding to position 85 in SEQ ID NO: 2;
vv) A, Q at a residue corresponding to position 86 in SEQ ID NO: 2;
ww) R at a residue corresponding to position 87 in SEQ ID NO: 2;
xx) L at a residue corresponding to position 88 in SEQ ID NO: 2;
yy) K at a residue corresponding to position 92 in SEQ ID NO: 2;
zz) G at a residue corresponding to position 93 in SEQ ID NO: 2;
aaa) R at a residue corresponding to position 94 in SEQ ID NO: 2;
bbb) R at a residue corresponding to position 95 in SEQ ID NO: 2;
ccc) A at a residue corresponding to position 97 in SEQ ID NO: 2;
ddd) M at a residue corresponding to position 98 in SEQ ID NO: 2;
eee) L at a residue corresponding to position 99 in SEQ ID NO: 2;
fff) C at a residue corresponding to position 100 in SEQ ID NO: 2;
ggg) P at a residue corresponding to position 101 in SEQ ID NO: 2;
hhh) R at a residue corresponding to position 103 in SEQ ID NO: 2;
iii) L, W, A at a residue corresponding to position 104 in SEQ ID NO: 2;
jjj) W, F at a residue corresponding to position 106 in SEQ ID NO: 2;
kkk) V, P, I, L, W, F at a residue corresponding to position 107 in SEQ ID NO: 2;
lll) V at a residue corresponding to position 108 in SEQ ID NO: 2;
mmm) V at a residue corresponding to position 109 in SEQ ID NO: 2;
nnn) M, F, Y, A, D at a residue corresponding to position 110 in SEQ ID NO: 2;
ooo) T, C, V at a residue corresponding to position 113 in SEQ ID NO: 2;
ppp) M, W, L, G, A at a residue corresponding to position 114 in SEQ ID NO: 2;
qqq) A at a residue corresponding to position 115 in SEQ ID NO: 2;
rrr) I at a residue corresponding to position 116 in SEQ ID NO: 2;
sss) V, T, I at a residue corresponding to position 119 in SEQ ID NO: 2;
ttt) M, F, V, A, L, W, Y at a residue corresponding to position 120 in SEQ ID NO: 2;
uuu) T at a residue corresponding to position 121 in SEQ ID NO: 2;
vvv) A at a residue corresponding to position 122 in SEQ ID NO: 2;
www) M, V, I at a residue corresponding to position 123 in SEQ ID NO: 2;
xxx) Y, S, N at a residue corresponding to position 124 in SEQ ID NO: 2;
yyy) P, T, W at a residue corresponding to position 125 in SEQ ID NO: 2;
zzz) M, N, R, A at a residue corresponding to position 126 in SEQ ID NO: 2;
aaaa) F, W, L at a residue corresponding to position 127 in SEQ ID NO: 2;
bbbb) R at a residue corresponding to position 128 in SEQ ID NO: 2;
cccc) M at a residue corresponding to position 130 in SEQ ID NO: 2;
dddd) E at a residue corresponding to position 131 in SEQ ID NO: 2;
eeee) E, Q, K at a residue corresponding to position 136 in SEQ ID NO: 2;
ffff) M at a residue corresponding to position 137 in SEQ ID NO: 2;
gggg) M, C, I at a residue corresponding to position 140 in SEQ ID NO: 2;
hhhh) M at a residue corresponding to position 141 in SEQ ID NO: 2;
iiii) N, Q at a residue corresponding to position 142 in SEQ ID NO: 2;
jjjj) G, R, D at a residue corresponding to position 145 in SEQ ID NO: 2;
kkkk) S at a residue corresponding to position 147 in SEQ ID NO: 2;
llll) L, V, A at a residue corresponding to position 148 in SEQ ID NO: 2;
mmmm) V, L at a residue corresponding to position 149 in SEQ ID NO: 2;
nnnn) V at a residue corresponding to position 150 in SEQ ID NO: 2;
oooo) C at a residue corresponding to position 151 in SEQ ID NO: 2;
pppp) L at a residue corresponding to position 157 in SEQ ID NO: 2;
qqqq) R, E at a residue corresponding to position 159 in SEQ ID NO: 2;
rrrr) I at a residue corresponding to position 161 in SEQ ID NO: 2;
ssss) V at a residue corresponding to position 163 in SEQ ID NO: 2;
tttt) A at a residue corresponding to position 164 in SEQ ID NO: 2;
uuuu) R at a residue corresponding to position 165 in SEQ ID NO: 2;
vvvv) R at a residue corresponding to position 167 in SEQ ID NO: 2;
wwww) H at a residue corresponding to position 168 in SEQ ID NO: 2;
xxxx) I at a residue corresponding to position 169 in SEQ ID NO: 2;
yyyy) I, V, L at a residue corresponding to position 170 in SEQ ID NO: 2;
zzzz) V at a residue corresponding to position 173 in SEQ ID NO: 2;
aaaaa) R at a residue corresponding to position 175 in SEQ ID NO: 2;
bbbbb) D at a residue corresponding to position 176 in SEQ ID NO: 2;
ccccc) E at a residue corresponding to position 177 in SEQ ID NO: 2;
ddddd) E at a residue corresponding to position 181 in SEQ ID NO: 2;
eeeee) I at a residue corresponding to position 183 in SEQ ID NO: 2;
fffff) S at a residue corresponding to position 185 in SEQ ID NO: 2;
ggggg) Y at a residue corresponding to position 186 in SEQ ID NO: 2;
hhhhh) D at a residue corresponding to position 187 in SEQ ID NO: 2;
iiiii) V at a residue corresponding to position 189 in SEQ ID NO: 2;
jjjjj) V at a residue corresponding to position 190 in SEQ ID NO: 2;
kkkkk) A, E at a residue corresponding to position 191 in SEQ ID NO: 2;
lllll) T at a residue corresponding to position 192 in SEQ ID NO: 2;
mmmmm) A at a residue corresponding to position 193 in SEQ ID NO: 2;
nnnnn) S at a residue corresponding to position 194 in SEQ ID NO: 2;
ooooo) S at a residue corresponding to position 195 in SEQ ID NO: 2;
ppppp) P at a residue corresponding to position 196 in SEQ ID NO: 2;
qqqqq) A, P, L at a residue corresponding to position 201 in SEQ ID NO: 2;
rrrrr) T at a residue corresponding to position 205 in SEQ ID NO: 2;
sssss) D at a residue corresponding to position 208 in SEQ ID NO: 2;
ttttt) I, T, P, V at a residue corresponding to position 209 in SEQ ID NO: 2;
uuuuu) L, T, C, M, V, A at a residue corresponding to position 211 in SEQ ID NO: 2;
vvvvv) L, M at a residue corresponding to position 212 in SEQ ID NO: 2;
wwwww) I, C, V, Q, M at a residue corresponding to position 213 in SEQ ID NO: 2;
xxxxx) Y at a residue corresponding to position 214 in SEQ ID NO: 2;
yyyyy) S at a residue corresponding to position 215 in SEQ ID NO: 2;
zzzzz) G at a residue corresponding to position 216 in SEQ ID NO: 2;
aaaaaa) T at a residue corresponding to position 218 in SEQ ID NO: 2;
bbbbbb) T at a residue corresponding to position 219 in SEQ ID NO: 2;
cccccc) G at a residue corresponding to position 220 in SEQ ID NO: 2;
dddddd) T, M, L, R, K, H at a residue corresponding to position 221 in SEQ ID NO: 2;
eeeeee) S at a residue corresponding to position 222 in SEQ ID NO: 2;
ffffff) A at a residue corresponding to position 224 in SEQ ID NO: 2;
gggggg) A at a residue corresponding to position 225 in SEQ ID NO: 2;
hhhhhh) M, I, V at a residue corresponding to position 226 in SEQ ID NO: 2;
iiiiii) L, D, H, I at a residue corresponding to position 227 in SEQ ID NO: 2;
jjjjjj) S at a residue corresponding to position 228 in SEQ ID NO: 2;
kkkkkk) H, K at a residue corresponding to position 230 in SEQ ID NO: 2;
llllll) N at a residue corresponding to position 231 in SEQ ID NO: 2;
mmmmmm) L, I, V at a residue corresponding to position 232 in SEQ ID NO: 2;
nnnnnn) L, V, M, W, A, I at a residue corresponding to position 233 in SEQ ID NO: 2;
oooooo) S, W at a residue corresponding to position 234 in SEQ ID NO: 2;
pppppp) Q, S at a residue corresponding to position 235 in SEQ ID NO: 2;
qqqqqq) A, C, I, M, V at a residue corresponding to position 236 in SEQ ID NO: 2;
rrrrrr) A, M, Q, R, E at a residue corresponding to position 237 in SEQ ID NO: 2;
ssssss) M, S, W, N, H at a residue corresponding to position 238 in SEQ ID NO: 2;
tttttt) F, M, V, C, L at a residue corresponding to position 239 in SEQ ID NO: 2;
uuuuuu) A, R at a residue corresponding to position 240 in SEQ ID NO: 2;
vvvvvv) A, H, E, Q at a residue corresponding to position 241 in SEQ ID NO: 2;
wwwwww) V, C, T, W at a residue corresponding to position 242 in SEQ ID NO: 2;
xxxxxx) I, F, M, W, V at a residue corresponding to position 243 in SEQ ID NO: 2;
yyyyyy) E, P, Q, H, G at a residue corresponding to position 244 in SEQ ID NO: 2;
zzzzzz) M, L, H, W, Y at a residue corresponding to position 245 in SEQ ID NO: 2;
aaaaaaa) I, T, M at a residue corresponding to position 246 in SEQ ID NO: 2;
bbbbbbb) R, P, E at a residue corresponding to position 247 in SEQ ID NO: 2;
ccccccc) E at a residue corresponding to position 251 in SEQ ID NO: 2;
ddddddd) H, V at a residue corresponding to position 252 in SEQ ID NO: 2;
eeeeeee) Y, M, F, L, V at a residue corresponding to position 253 in SEQ ID NO: 2;
fffffff) I, F, V, M at a residue corresponding to position 254 in SEQ ID NO: 2;
ggggggg) A, T, N, L, I, G, H, C, V, S at a residue corresponding to position 255 in SEQ ID NO: 2;
hhhhhhh) F, V, I, C, Y at a residue corresponding to position 256 in SEQ ID NO: 2;
iiiiiii) M at a residue corresponding to position 257 in SEQ ID NO: 2;
jjjjjjj) L, F at a residue corresponding to position 259 in SEQ ID NO: 2;
kkkkkkk) H, S, Y, F at a residue corresponding to position 260 in SEQ ID NO: 2;
lllllll) C, T, A, I, M at a residue corresponding to position 262 in SEQ ID NO: 2;
mmmmmmm) A, Y, H at a residue corresponding to position 263 in SEQ ID NO: 2;
nnnnnnn) A at a residue corresponding to position 264 in SEQ ID NO: 2;
ooooooo) M, L, W, F, C at a residue corresponding to position 265 in SEQ ID NO: 2;
ppppppp) N, T, M, V at a residue corresponding to position 266 in SEQ ID NO: 2;
qqqqqqq) I, G, M, W, F, L, V, C, A at a residue corresponding to position 267 in SEQ ID NO: 2;
rrrrrrr) M, A, C, G, V at a residue corresponding to position 268 in SEQ ID NO: 2;
sssssss) L, M, I, V at a residue corresponding to position 269 in SEQ ID NO: 2;
ttttttt) M, H, Y, T at a residue corresponding to position 270 in SEQ ID NO: 2;
uuuuuuu) A, P, C, T, L at a residue corresponding to position 271 in SEQ ID NO: 2;
vvvvvvv) C, V, P, A, T at a residue corresponding to position 272 in SEQ ID NO: 2;
wwwwwww) M, V, F, I at a residue corresponding to position 273 in SEQ ID NO: 2;
xxxxxxx) Y, R, V, M, C, H, W at a residue corresponding to position 274 in SEQ ID NO: 2;
yyyyyyy) G, C, S, H at a residue corresponding to position 275 in SEQ ID NO: 2;
zzzzzzz) A, C at a residue corresponding to position 277 in SEQ ID NO: 2;
aaaaaaaa) C, T, A, H at a residue corresponding to position 278 in SEQ ID NO: 2;
bbbbbbbb) V, M, I at a residue corresponding to position 279 in SEQ ID NO: 2;
cccccccc) F, L, V, M at a residue corresponding to position 281 in SEQ ID NO: 2;
dddddddd) P, M, E, A, H at a residue corresponding to position 282 in SEQ ID NO: 2;
eeeeeeee) E, D, P, R at a residue corresponding to position 283 in SEQ ID NO: 2;
ffffffff) F, R, W at a residue corresponding to position 284 in SEQ ID NO: 2;
gggggggg) R, F, W, D at a residue corresponding to position 285 in SEQ ID NO: 2;
hhhhhhhh) D, F at a residue corresponding to position 286 in SEQ ID NO: 2;
iiiiiiii) P, A, V at a residue corresponding to position 287 in SEQ ID NO: 2;
jjjjjjjj) A at a residue corresponding to position 290 in SEQ ID NO: 2;
kkkkkkkk) M, V, L, P, I at a residue corresponding to position 291 in SEQ ID NO: 2;
llllllll) H at a residue corresponding to position 292 in SEQ ID NO: 2;
mmmmmmmm) I, L, A at a residue corresponding to position 294 in SEQ ID NO: 2;
nnnnnnnn) W, M, V, I at a residue corresponding to position 295 in SEQ ID NO: 2;
oooooooo) D, Q at a residue corresponding to position 296 in SEQ ID NO: 2;
pppppppp) M, V, L at a residue corresponding to position 298 in SEQ ID NO: 2;
qqqqqqqq) A at a residue corresponding to position 299 in SEQ ID NO: 2;
rrrrrrrr) Q at a residue corresponding to position 300 in SEQ ID NO: 2;
ssssssss) Q at a residue corresponding to position 302 in SEQ ID NO: 2;
tttttttt) V, I, A, P, C at a residue corresponding to position 303 in SEQ ID NO: 2;
uuuuuuuu) S at a residue corresponding to position 304 in SEQ ID NO: 2;
vvvvvvvv) W, Y, V, I, F, H, M at a residue corresponding to position 305 in SEQ ID NO: 2;
wwwwwwww) V, Y, I, M, L, F at a residue corresponding to position 306 in SEQ ID NO: 2;
xxxxxxxx) P, W, Y, H, M at a residue corresponding to position 307 in SEQ ID NO: 2;
yyyyyyyy) T, A, S, L, G, V, M at a residue corresponding to position 308 in SEQ ID NO: 2;
zzzzzzzz) A, R, P, T at a residue corresponding to position 311 in SEQ ID NO: 2;
aaaaaaaaa) I, M, V, F at a residue corresponding to position 312 in SEQ ID NO: 2;
bbbbbbbbb) L, F, W at a residue corresponding to position 313 in SEQ ID NO: 2;
ccccccccc) Q, E, H at a residue corresponding to position 314 in SEQ ID NO: 2;
ddddddddd) R, A, K, L at a residue corresponding to position 315 in SEQ ID NO: 2;
eeeeeeeee) W, M, I, F at a residue corresponding to position 316 in SEQ ID NO: 2;
fffffffff) H, D at a residue corresponding to position 318 in SEQ ID NO: 2;
ggggggggg) R, Q at a residue corresponding to position 321 in SEQ ID NO: 2;
hhhhhhhhh) Q, K, A, H at a residue corresponding to position 322 in SEQ ID NO: 2;
iiiiiiiii) L, F at a residue corresponding to position 330 in SEQ ID NO: 2;
jjjjjjjjj) G at a residue corresponding to position 331 in SEQ ID NO: 2;
kkkkkkkkk) S at a residue corresponding to position 333 in SEQ ID NO: 2;
lllllllll) I, M, V at a residue corresponding to position 334 in SEQ ID NO: 2;
mmmmmmmmm) R, K at a residue corresponding to position 335 in SEQ ID NO: 2;
nnnnnnnnn) T, I, M, Y, V, F, W at a residue corresponding to position 336 in SEQ ID NO: 2;
ooooooooo) L, M, F, A, V at a residue corresponding to position 337 in SEQ ID NO: 2;
ppppppppp) W, L, M, V at a residue corresponding to position 338 in SEQ ID NO: 2;
qqqqqqqqq) F, T, S, C at a residue corresponding to position 339 in SEQ ID NO: 2;
rrrrrrrrr) G at a residue corresponding to position 340 in SEQ ID NO: 2;
sssssssss) A, S at a residue corresponding to position 341 in SEQ ID NO: 2;
ttttttttt) S at a residue corresponding to position 342 in SEQ ID NO: 2;
uuuuuuuuu) P, A at a residue corresponding to position 343 in SEQ ID NO: 2;
vvvvvvvvv) M, I, V, C at a residue corresponding to position 344 in SEQ ID NO: 2;
wwwwwwwww) S at a residue corresponding to position 345 in SEQ ID NO: 2;
xxxxxxxxx) V, A, P, E at a residue corresponding to position 346 in SEQ ID NO: 2;
yyyyyyyyy) E at a residue corresponding to position 347 in SEQ ID NO: 2;
zzzzzzzzz) T, I, L at a residue corresponding to position 348 in SEQ ID NO: 2;
aaaaaaaaaa) M, F, L, A, I, Y at a residue corresponding to position 349 in SEQ ID NO: 2;
bbbbbbbbbb) F, R, Q, W at a residue corresponding to position 351 in SEQ ID NO: 2;
cccccccccc) I, M, Y at a residue corresponding to position 352 in SEQ ID NO: 2;
dddddddddd) R at a residue corresponding to position 353 in SEQ ID NO: 2;
eeeeeeeeee) K at a residue corresponding to position 354 in SEQ ID NO: 2;
ffffffffff) A, R at a residue corresponding to position 355 in SEQ ID NO: 2;
gggggggggg) W, G, V, L, P, I, F, A at a residue corresponding to position 357 in SEQ ID NO: 2;
hhhhhhhhhh) Q at a residue corresponding to position 358 in SEQ ID NO: 2;
iiiiiiiiii) H, Q at a residue corresponding to position 359 in SEQ ID NO: 2;
jjjjjjjjjj) F at a residue corresponding to position 361 in SEQ ID NO: 2;
kkkkkkkkkk) H, L, C at a residue corresponding to position 362 in SEQ ID NO: 2;
llllllllll) D, V, I, G at a residue corresponding to position 363 in SEQ ID NO: 2;
mmmmmmmmmm) M, V, I at a residue corresponding to position 365 in SEQ ID NO: 2;
nnnnnnnnnn) I, V, L, F at a residue corresponding to position 366 in SEQ ID NO: 2;
oooooooooo) T, C, M, I, V, L at a residue corresponding to position 367 in SEQ ID NO: 2;
pppppppppp) N, Q, E, T at a residue corresponding to position 368 in SEQ ID NO: 2;
qqqqqqqqqq) G, A at a residue corresponding to position 369 in SEQ ID NO: 2;
rrrrrrrrrr) Y, F at a residue corresponding to position 370 in SEQ ID NO: 2;
ssssssssss) L, Q at a residue corresponding to position 372 in SEQ ID NO: 2;
tttttttttt) S at a residue corresponding to position 373 in SEQ ID NO: 2;
uuuuuuuuuu) A, C at a residue corresponding to position 375 in SEQ ID NO: 2;
vvvvvvvvvv) T, S, C, G at a residue corresponding to position 376 in SEQ ID NO: 2;
wwwwwwwwww) G, M at a residue corresponding to position 377 in SEQ ID NO: 2;
xxxxxxxxxx) C, T, V at a residue corresponding to position 378 in SEQ ID NO: 2;
yyyyyyyyyy) C, V, I, L, A at a residue corresponding to position 379 in SEQ ID NO: 2;
zzzzzzzzzz) F, C, H, S, M, T, V at a residue corresponding to position 380 in SEQ ID NO: 2;
aaaaaaaaaaa) N, F, L, C, V at a residue corresponding to position 381 in SEQ ID NO: 2;
bbbbbbbbbbb) L, N, C, T, P at a residue corresponding to position 382 in SEQ ID NO: 2;
ccccccccccc) D, N, R, P, Y at a residue corresponding to position 383 in SEQ ID NO: 2;
ddddddddddd) P, N, D, W at a residue corresponding to position 384 in SEQ ID NO: 2;
eeeeeeeeeee) G, D at a residue corresponding to position 385 in SEQ ID NO: 2;
fffffffffff) D, V, L, T at a residue corresponding to position 386 in SEQ ID NO: 2;
ggggggggggg) D, R, N, H, E at a residue corresponding to position 387 in SEQ ID NO: 2;
hhhhhhhhhhh) K, H at a residue corresponding to position 388 in SEQ ID NO: 2;
iiiiiiiiiii) A, P, L at a residue corresponding to position 389 in SEQ ID NO: 2;
jjjjjjjjjjj) S, T at a residue corresponding to position 391 in SEQ ID NO: 2;
kkkkkkkkkkk) V, C at a residue corresponding to position 392 in SEQ ID NO: 2;
lllllllllll) R, K, I, Q, L at a residue corresponding to position 394 in SEQ ID NO: 2;
mmmmmmmmmmm) A at a residue corresponding to position 395 in SEQ ID NO: 2;
nnnnnnnnnnn) F, I, M, V at a residue corresponding to position 396 in SEQ ID NO: 2;
ooooooooooo) D, H, N at a residue corresponding to position 398 in SEQ ID NO: 2;
ppppppppppp) M, V, T, C, I at a residue corresponding to position 399 in SEQ ID NO: 2;
qqqqqqqqqqq) D, Q at a residue corresponding to position 400 in SEQ ID NO: 2;
rrrrrrrrrrr) M, I, C, L, A at a residue corresponding to position 401 in SEQ ID NO: 2;
sssssssssss) R at a residue corresponding to position 402 in SEQ ID NO: 2;
ttttttttttt) V at a residue corresponding to position 404 in SEQ ID NO: 2;
uuuuuuuuuuu) D at a residue corresponding to position 405 in SEQ ID NO: 2;
vvvvvvvvvvv) E at a residue corresponding to position 406 in SEQ ID NO: 2;
wwwwwwwwwww) P at a residue corresponding to position 407 in SEQ ID NO: 2;
xxxxxxxxxxx) Q, E, G at a residue corresponding to position 409 in SEQ ID NO: 2;
yyyyyyyyyyy) D, S, V, G, N at a residue corresponding to position 411 in SEQ ID NO: 2;
zzzzzzzzzzz) I, V at a residue corresponding to position 413 in SEQ ID NO: 2;
aaaaaaaaaaaa) W, L, V, C, Q at a residue corresponding to position 414 in SEQ ID NO: 2;
bbbbbbbbbbbb) I at a residue corresponding to position 415 in SEQ ID NO: 2;
cccccccccccc) R at a residue corresponding to position 416 in SEQ ID NO: 2;
dddddddddddd) S at a residue corresponding to position 417 in SEQ ID NO: 2;
eeeeeeeeeeee) D at a residue corresponding to position 418 in SEQ ID NO: 2;
ffffffffffff) H, S, Q, M at a residue corresponding to position 419 in SEQ ID NO: 2;
gggggggggggg) V at a residue corresponding to position 420 in SEQ ID NO: 2;
hhhhhhhhhhhh) T, F at a residue corresponding to position 421 in SEQ ID NO: 2;
iiiiiiiiiiii) H, L at a residue corresponding to position 425 in SEQ ID NO: 2;
jjjjjjjjjjjj) A at a residue corresponding to position 430 in SEQ ID NO: 2;
kkkkkkkkkkkk) K at a residue corresponding to position 432 in SEQ ID NO: 2;
llllllllllll) R, D at a residue corresponding to position 433 in SEQ ID NO: 2;
mmmmmmmmmmmm) T, V at a residue corresponding to position 434 in SEQ ID NO: 2;
nnnnnnnnnnnn) I, M, L at a residue corresponding to position 435 in SEQ ID NO: 2;
oooooooooooo) F, M, Y at a residue corresponding to position 441 in SEQ ID NO: 2;
pppppppppppp) K, H, Y, R, C at a residue corresponding to position 442 in SEQ ID NO: 2;
qqqqqqqqqqqq) S at a residue corresponding to position 443 in SEQ ID NO: 2;
rrrrrrrrrrrr) M, I, L, V at a residue corresponding to position 446 in SEQ ID NO: 2;
ssssssssssss) A, G at a residue corresponding to position 447 in SEQ ID NO: 2;
tttttttttttt) I at a residue corresponding to position 449 in SEQ ID NO: 2;
uuuuuuuuuuuu) D, M at a residue corresponding to position 450 in SEQ ID NO: 2;
vvvvvvvvvvvv) E at a residue corresponding to position 451 in SEQ ID NO: 2;
wwwwwwwwwwww) P at a residue corresponding to position 452 in SEQ ID NO: 2;
xxxxxxxxxxxx) G at a residue corresponding to position 453 in SEQ ID NO: 2;
yyyyyyyyyyyy) E, G, R at a residue corresponding to position 455 in SEQ ID NO: 2;
zzzzzzzzzzzz) D, E, Q, N at a residue corresponding to position 456 in SEQ ID NO: 2;
aaaaaaaaaaaaa) N, F, Y, W at a residue corresponding to position 458 in SEQ ID NO: 2;
bbbbbbbbbbbbb) Y at a residue corresponding to position 459 in SEQ ID NO: 2;
ccccccccccccc) Y, F, I, R, W at a residue corresponding to position 460 in SEQ ID NO: 2;
ddddddddddddd) I, L, Y at a residue corresponding to position 461 in SEQ ID NO: 2;
eeeeeeeeeeeee) I, T, V at a residue corresponding to position 462 in SEQ ID NO: 2;
fffffffffffff) D at a residue corresponding to position 463 in SEQ ID NO: 2;
ggggggggggggg) D at a residue corresponding to position 464 in SEQ ID NO: 2;
hhhhhhhhhhhhh) L at a residue corresponding to position 465 in SEQ ID NO: 2;
iiiiiiiiiiiii) D, K, A at a residue corresponding to position 466 in SEQ ID NO: 2;
jjjjjjjjjjjjj) K at a residue corresponding to position 467 in SEQ ID NO: 2;
kkkkkkkkkkkkk) M at a residue corresponding to position 468 in SEQ ID NO: 2;
lllllllllllll) L at a residue corresponding to position 469 in SEQ ID NO: 2;
mmmmmmmmmmmmm) V at a residue corresponding to position 470 in SEQ ID NO: 2;
nnnnnnnnnnnnn) T, V at a residue corresponding to position 471 in SEQ ID NO: 2;
ooooooooooooo) S, N at a residue corresponding to position 473 in SEQ ID NO: 2;
ppppppppppppp) E at a residue corresponding to position 474 in SEQ ID NO: 2;
qqqqqqqqqqqqq) N, Y, L, F, V, I at a residue corresponding to position 476 in SEQ ID NO: 2;
rrrrrrrrrrrrr) T, L at a residue corresponding to position 478 in SEQ ID NO: 2;
sssssssssssss) P, V at a residue corresponding to position 479 in SEQ ID NO: 2;
ttttttttttttt) L, I, S, P, A, V at a residue corresponding to position 480 in SEQ ID NO: 2;
uuuuuuuuuuuuu) P, E at a residue corresponding to position 481 in SEQ ID NO: 2;
vvvvvvvvvvvvv) E at a residue corresponding to position 483 in SEQ ID NO: 2;
wwwwwwwwwwwww) A, L, E, R at a residue corresponding to position 485 in SEQ ID NO: 2;
xxxxxxxxxxxxx) C at a residue corresponding to position 490 in SEQ ID NO: 2;
yyyyyyyyyyyyy) P at a residue corresponding to position 492 in SEQ ID NO: 2;
zzzzzzzzzzzzz) F at a residue corresponding to position 493 in SEQ ID NO: 2;
aaaaaaaaaaaaaa) H at a residue corresponding to position 496 in SEQ ID NO: 2;
bbbbbbbbbbbbbb) A at a residue corresponding to position 497 in SEQ ID NO: 2;
cccccccccccccc) C at a residue corresponding to position 498 in SEQ ID NO: 2;
dddddddddddddd) P at a residue corresponding to position 502 in SEQ ID NO: 2;
eeeeeeeeeeeeee) H at a residue corresponding to position 504 in SEQ ID NO: 2;
ffffffffffffff) Y, V, W at a residue corresponding to position 507 in SEQ ID NO: 2;
gggggggggggggg) V at a residue corresponding to position 508 in SEQ ID NO: 2;
hhhhhhhhhhhhhh) A at a residue corresponding to position 510 in SEQ ID NO: 2;
iiiiiiiiiiiiii) V, L at a residue corresponding to position 511 in SEQ ID NO: 2;
jjjjjjjjjjjjjj) I at a residue corresponding to position 512 in SEQ ID NO: 2;
kkkkkkkkkkkkkk) V at a residue corresponding to position 513 in SEQ ID NO: 2;
llllllllllllll) N at a residue corresponding to position 514 in SEQ ID NO: 2;
mmmmmmmmmmmmmm) D, S at a residue corresponding to position 515 in SEQ ID NO: 2;
nnnnnnnnnnnnnn) A at a residue corresponding to position 516 in SEQ ID NO: 2;
oooooooooooooo) I at a residue corresponding to position 518 in SEQ ID NO: 2;
pppppppppppppp) T at a residue corresponding to position 519 in SEQ ID NO: 2;
qqqqqqqqqqqqqq) P, W at a residue corresponding to position 521 in SEQ ID NO: 2;
rrrrrrrrrrrrrr) L, M at a residue corresponding to position 526 in SEQ ID NO: 2;
ssssssssssssss) L at a residue corresponding to position 527 in SEQ ID NO: 2;
tttttttttttttt) R, H at a residue corresponding to position 532 in SEQ ID NO: 2;
uuuuuuuuuuuuuu) L at a residue corresponding to position 535 in SEQ ID NO: 2;
vvvvvvvvvvvvvv) Q at a residue corresponding to position 540 in SEQ ID NO: 2;
wwwwwwwwwwwwww) Y, W at a residue corresponding to position 542 in SEQ ID NO: 2;
xxxxxxxxxxxxxx) N at a residue corresponding to position 546 in SEQ ID NO: 2;
yyyyyyyyyyyyyy) Q at a residue corresponding to position 552 in SEQ ID NO: 2;
zzzzzzzzzzzzzz) E at a residue corresponding to position 554 in SEQ ID NO: 2;
aaaaaaaaaaaaaaa) F at a residue corresponding to position 558 in SEQ ID NO: 2;
bbbbbbbbbbbbbbb) D at a residue corresponding to position 560 in SEQ ID NO: 2;
ccccccccccccccc) D at a residue corresponding to position 561 in SEQ ID NO: 2;
ddddddddddddddd) F, L, W at a residue corresponding to position 563 in SEQ ID NO: 2;
eeeeeeeeeeeeeee) D, K, S, L, T, R at a residue corresponding to position 564 in SEQ ID NO: 2;
fffffffffffffff) M, I, V, P, T at a residue corresponding to position 565 in SEQ ID NO: 2;
ggggggggggggggg) A, E, D, P at a residue corresponding to position 566 in SEQ ID NO: 2;
hhhhhhhhhhhhhhh) K, Q, T, N, S, A at a residue corresponding to position 567 in SEQ ID NO: 2;
iiiiiiiiiiiiiii) G, N, D at a residue corresponding to position 568 in SEQ ID NO: 2;
jjjjjjjjjjjjjjj) M, K at a residue corresponding to position 569 in SEQ ID NO: 2;
kkkkkkkkkkkkkkk) V, L at a residue corresponding to position 570 in SEQ ID NO: 2;
lllllllllllllll) R, P at a residue corresponding to position 572 in SEQ ID NO: 2;
mmmmmmmmmmmmmmm) T, S at a residue corresponding to position 573 in SEQ ID NO: 2;
nnnnnnnnnnnnnnn) M, L at a residue corresponding to position 574 in SEQ ID NO: 2;
ooooooooooooooo) A, Y, K, T at a residue corresponding to position 575 in SEQ ID NO: 2;
ppppppppppppppp) Y, A, L at a residue corresponding to position 577 in SEQ ID NO: 2;
qqqqqqqqqqqqqqq) Q, A at a residue corresponding to position 578 in SEQ ID NO: 2;
rrrrrrrrrrrrrrr) D at a residue corresponding to position 583 in SEQ ID NO: 2;
sssssssssssssss) N at a residue corresponding to position 584 in SEQ ID NO: 2;
ttttttttttttttt) H, L at a residue corresponding to position 585 in SEQ ID NO: 2;
uuuuuuuuuuuuuuu) A at a residue corresponding to position 587 in SEQ ID NO: 2;
vvvvvvvvvvvvvvv) Q at a residue corresponding to position 588 in SEQ ID NO: 2;
wwwwwwwwwwwwwww) Q, E at a residue corresponding to position 591 in SEQ ID NO: 2;
xxxxxxxxxxxxxxx) A at a residue corresponding to position 593 in SEQ ID NO: 2;
yyyyyyyyyyyyyyy) E at a residue corresponding to position 595 in SEQ ID NO: 2;
zzzzzzzzzzzzzzz) K at a residue corresponding to position 596 in SEQ ID NO: 2;
aaaaaaaaaaaaaaaa) D at a residue corresponding to position 598 in SEQ ID NO: 2;
bbbbbbbbbbbbbbbb) S at a residue corresponding to position 599 in SEQ ID NO: 2;
cccccccccccccccc) L at a residue corresponding to position 601 in SEQ ID NO: 2; or
dddddddddddddddd) any combination thereof.

26. The engineered ACL of claim 25, wherein the one or more amino acid alterations result in an engineered ACL comprising:
a) E, R at a residue corresponding to position 5 in SEQ ID NO: 2;
b) L at a residue corresponding to position 6 in SEQ ID NO: 2;
c) K, T at a residue corresponding to position 7 in SEQ ID NO: 2;
d) P at a residue corresponding to position 8 in SEQ ID NO: 2;
e) D, N, R, P, K at a residue corresponding to position 10 in SEQ ID NO: 2;
f) A, V, M, I at a residue corresponding to position 12 in SEQ ID NO: 2;
g) C at a residue corresponding to position 13 in SEQ ID NO: 2;
h) Q, P, A at a residue corresponding to position 14 in SEQ ID NO: 2;
i) S, E at a residue corresponding to position 15 in SEQ ID NO: 2;
j) I, C at a residue corresponding to position 16 in SEQ ID NO: 2;
k) D, R at a residue corresponding to position 19 in SEQ ID NO: 2;
l) H, K, R, D at a residue corresponding to position 20 in SEQ ID NO: 2;
m) E, D at a residue corresponding to position 22 in SEQ ID NO: 2;
n) N at a residue corresponding to position 25 in SEQ ID NO: 2;
o) M at a residue corresponding to position 28 in SEQ ID NO: 2;
p) C at a residue corresponding to position 37 in SEQ ID NO: 2;
q) R at a residue corresponding to position 39 in SEQ ID NO: 2;
r) T at a residue corresponding to position 40 in SEQ ID NO: 2;
s) V, I, M at a residue corresponding to position 41 in SEQ ID NO: 2;
t) S at a residue corresponding to position 43 in SEQ ID NO: 2;
u) I, V at a residue corresponding to position 45 in SEQ ID NO: 2;
v) R, L, I, V, F at a residue corresponding to position 50 in SEQ ID NO: 2;
w) R at a residue corresponding to position 51 in SEQ ID NO: 2;
x) L at a residue corresponding to position 53 in SEQ ID NO: 2;
y) F at a residue corresponding to position 57 in SEQ ID NO: 2;
z) F, M, L, V at a residue corresponding to position 58 in SEQ ID NO: 2;
aa) V at a residue corresponding to position 59 in SEQ ID NO: 2;
bb) P at a residue corresponding to position 61 in SEQ ID NO: 2;
cc) D at a residue corresponding to position 62 in SEQ ID NO: 2;
dd) D at a residue corresponding to position 63 in SEQ ID NO: 2;
ee) K at a residue corresponding to position 67 in SEQ ID NO: 2;
ff) F, L, Y at a residue corresponding to position 69 in SEQ ID NO: 2;
gg) W, F at a residue corresponding to position 71 in SEQ ID NO: 2;
hh) A, R at a residue corresponding to position 72 in SEQ ID NO: 2;
ii) F at a residue corresponding to position 74 in SEQ ID NO: 2;
jj) D at a residue corresponding to position 75 in SEQ ID NO: 2;
kk) D, T, A, R at a residue corresponding to position 76 in SEQ ID NO: 2;
ll) R at a residue corresponding to position 77 in SEQ ID NO: 2;
mm) A at a residue corresponding to position 78 in SEQ ID NO: 2;
nn) K at a residue corresponding to position 80 in SEQ ID NO: 2;
oo) C at a residue corresponding to position 81 in SEQ ID NO: 2;
pp) S, N at a residue corresponding to position 83 in SEQ ID NO: 2;
qq) Y, H at a residue corresponding to position 84 in SEQ ID NO: 2;
rr) F at a residue corresponding to position 85 in SEQ ID NO: 2;
ss) Q, A at a residue corresponding to position 86 in SEQ ID NO: 2;
tt) R at a residue corresponding to position 87 in SEQ ID NO: 2;
uu) L at a residue corresponding to position 88 in SEQ ID NO: 2;
vv) K at a residue corresponding to position 92 in SEQ ID NO: 2;
ww) G at a residue corresponding to position 93 in SEQ ID NO: 2;
xx) R at a residue corresponding to position 94 in SEQ ID NO: 2;
yy) R at a residue corresponding to position 95 in SEQ ID NO: 2;
zz) A at a residue corresponding to position 97 in SEQ ID NO: 2;
aaa) M at a residue corresponding to position 98 in SEQ ID NO: 2;
bbb) L at a residue corresponding to position 99 in SEQ ID NO: 2;
ccc) C at a residue corresponding to position 100 in SEQ ID NO: 2;
ddd) P at a residue corresponding to position 101 in SEQ ID NO: 2;
eee) R at a residue corresponding to position 103 in SEQ ID NO: 2;
fff) A, L, W at a residue corresponding to position 104 in SEQ ID NO: 2;
ggg) F at a residue corresponding to position 106 in SEQ ID NO: 2;
hhh) V at a residue corresponding to position 108 in SEQ ID NO: 2;
iii) V at a residue corresponding to position 109 in SEQ ID NO: 2;
jjj) A, M at a residue corresponding to position 110 in SEQ ID NO: 2;
kkk) C at a residue corresponding to position 113 in SEQ ID NO: 2;
lll) I at a residue corresponding to position 116 in SEQ ID NO: 2;
mmm) T, V, I at a residue corresponding to position 119 in SEQ ID NO: 2;
nnn) Y, V, F, L, M, A at a residue corresponding to position 120 in SEQ ID NO: 2;
ooo) T at a residue corresponding to position 121 in SEQ ID NO: 2;
ppp) I, M, V at a residue corresponding to position 123 in SEQ ID NO: 2;
qqq) S, N, Y at a residue corresponding to position 124 in SEQ ID NO: 2;
rrr) P, T, W at a residue corresponding to position 125 in SEQ ID NO: 2;
sss) R, A, N, M at a residue corresponding to position 126 in SEQ ID NO: 2;
ttt) L, W, F at a residue corresponding to position 127 in SEQ ID NO: 2;
uuu) R at a residue corresponding to position 128 in SEQ ID NO: 2;
vvv) M at a residue corresponding to position 130 in SEQ ID NO: 2;
www) E at a residue corresponding to position 131 in SEQ ID NO: 2;
xxx) E, K, Q at a residue corresponding to position 136 in SEQ ID NO: 2;
yyy) M at a residue corresponding to position 137 in SEQ ID NO: 2;
zzz) M, I, C at a residue corresponding to position 140 in SEQ ID NO: 2;
aaaa) M at a residue corresponding to position 141 in SEQ ID NO: 2;
bbbb) Q, N at a residue corresponding to position 142 in SEQ ID NO: 2;
cccc) R, D, G at a residue corresponding to position 145 in SEQ ID NO: 2;
dddd) S at a residue corresponding to position 147 in SEQ ID NO: 2;
eeee) V, L, A at a residue corresponding to position 148 in SEQ ID NO: 2;
ffff) V at a residue corresponding to position 149 in SEQ ID NO: 2;
gggg) V at a residue corresponding to position 150 in SEQ ID NO: 2;
hhhh) C at a residue corresponding to position 151 in SEQ ID NO: 2;
iiii) E, R at a residue corresponding to position 159 in SEQ ID NO: 2;
jjjj) I at a residue corresponding to position 161 in SEQ ID NO: 2;
kkkk) V at a residue corresponding to position 163 in SEQ ID NO: 2;
llll) A at a residue corresponding to position 164 in SEQ ID NO: 2;
mmmm) R at a residue corresponding to position 165 in SEQ ID NO: 2;
nnnn) R at a residue corresponding to position 167 in SEQ ID NO: 2;
oooo) H at a residue corresponding to position 168 in SEQ ID NO: 2;
pppp) I at a residue corresponding to position 169 in SEQ ID NO: 2;
qqqq) I, L, V at a residue corresponding to position 170 in SEQ ID NO: 2;
rrrr) V at a residue corresponding to position 173 in SEQ ID NO: 2;
ssss) R at a residue corresponding to position 175 in SEQ ID NO: 2;
tttt) D at a residue corresponding to position 176 in SEQ ID NO: 2;
uuuu) E at a residue corresponding to position 177 in SEQ ID NO: 2;
vvvv) E at a residue corresponding to position 181 in SEQ ID NO: 2;
wwww) I at a residue corresponding to position 183 in SEQ ID NO: 2;
xxxx) S at a residue corresponding to position 185 in SEQ ID NO: 2;
yyyy) Y at a residue corresponding to position 186 in SEQ ID NO: 2;
zzzz) D at a residue corresponding to position 187 in SEQ ID NO: 2;
aaaaa) V at a residue corresponding to position 189 in SEQ ID NO: 2;
bbbbb) V at a residue corresponding to position 190 in SEQ ID NO: 2;
ccccc) E, A at a residue corresponding to position 191 in SEQ ID NO: 2;
ddddd) T at a residue corresponding to position 192 in SEQ ID NO: 2;
eeeee) A at a residue corresponding to position 193 in SEQ ID NO: 2;
fffff) S at a residue corresponding to position 194 in SEQ ID NO: 2;
ggggg) S at a residue corresponding to position 195 in SEQ ID NO: 2;
hhhhh) P at a residue corresponding to position 196 in SEQ ID NO: 2;
iiiii) L, A at a residue corresponding to position 201 in SEQ ID NO: 2;
jjjjj) T at a residue corresponding to position 205 in SEQ ID NO: 2;
kkkkk) D at a residue corresponding to position 208 in SEQ ID NO: 2;
lllll) I, T, V at a residue corresponding to position 209 in SEQ ID NO: 2;
mmmmm) L at a residue corresponding to position 211 in SEQ ID NO: 2;
nnnnn) L, M at a residue corresponding to position 212 in SEQ ID NO: 2;
ooooo) I, M, V at a residue corresponding to position 213 in SEQ ID NO: 2;
ppppp) Y at a residue corresponding to position 214 in SEQ ID NO: 2;
qqqqq) S at a residue corresponding to position 215 in SEQ ID NO: 2;
rrrrr) T at a residue corresponding to position 218 in SEQ ID NO: 2;
sssss) T at a residue corresponding to position 219 in SEQ ID NO: 2;
ttttt) G at a residue corresponding to position 220 in SEQ ID NO: 2;
uuuuu) K, L, T, H, R, M at a residue corresponding to position 221 in SEQ ID NO: 2;
vvvvv) S at a residue corresponding to position 222 in SEQ ID NO: 2;
wwwww) A at a residue corresponding to position 224 in SEQ ID NO: 2;
xxxxx) M, V, I at a residue corresponding to position 226 in SEQ ID NO: 2;
yyyyy) I at a residue corresponding to position 227 in SEQ ID NO: 2;
zzzzz) S at a residue corresponding to position 228 in SEQ ID NO: 2;
aaaaaa) H, K at a residue corresponding to position 230 in SEQ ID NO: 2;
bbbbbb) L at a residue corresponding to position 232 in SEQ ID NO: 2;
cccccc) A at a residue corresponding to position 233 in SEQ ID NO: 2;
dddddd) S at a residue corresponding to position 234 in SEQ ID NO: 2;
eeeeee) Q at a residue corresponding to position 235 in SEQ ID NO: 2;
ffffff) M at a residue corresponding to position 236 in SEQ ID NO: 2;
gggggg) A, M, Q at a residue corresponding to position 237 in SEQ ID NO: 2;
hhhhhh) M, H at a residue corresponding to position 238 in SEQ ID NO: 2;
iiiiii) V, L, M at a residue corresponding to position 239 in SEQ ID NO: 2;
jjjjjj) A at a residue corresponding to position 240 in SEQ ID NO: 2;
kkkkkk) Q, E, H, A at a residue corresponding to position 241 in SEQ ID NO: 2;
llllll) C, V, T at a residue corresponding to position 242 in SEQ ID NO: 2;
mmmmmm) W, F, M, I, V at a residue corresponding to position 243 in SEQ ID NO: 2;
nnnnnn) P, Q, G, H, E at a residue corresponding to position 244 in SEQ ID NO: 2;
oooooo) L, M, W at a residue corresponding to position 245 in SEQ ID NO: 2;
pppppp) I at a residue corresponding to position 246 in SEQ ID NO: 2;
qqqqqq) R, P at a residue corresponding to position 247 in SEQ ID NO: 2;
rrrrrr) V, H at a residue corresponding to position 252 in SEQ ID NO: 2;
ssssss) L, V, M at a residue corresponding to position 253 in SEQ ID NO: 2;
tttttt) M, I, V at a residue corresponding to position 254 in SEQ ID NO: 2;
uuuuuu) C, V, T, N at a residue corresponding to position 255 in SEQ ID NO: 2;
vvvvvv) Y, V, F at a residue corresponding to position 256 in SEQ ID NO: 2;
wwwwww) S, H, F, Y at a residue corresponding to position 260 in SEQ ID NO: 2;
xxxxxx) T, C, A at a residue corresponding to position 262 in SEQ ID NO: 2;
yyyyyy) Y at a residue corresponding to position 263 in SEQ ID NO: 2;
zzzzzz) A at a residue corresponding to position 264 in SEQ ID NO: 2;
aaaaaaa) M at a residue corresponding to position 265 in SEQ ID NO: 2;
bbbbbbb) N, M at a residue corresponding to position 266 in SEQ ID NO: 2;
ccccccc) M, F, L, W, G at a residue corresponding to position 267 in SEQ ID NO: 2;
ddddddd) M, A at a residue corresponding to position 268 in SEQ ID NO: 2;
eeeeeee) M at a residue corresponding to position 270 in SEQ ID NO: 2;
fffffff) L, C, A at a residue corresponding to position 271 in SEQ ID NO: 2;
ggggggg) A, V, T, P, C at a residue corresponding to position 272 in SEQ ID NO: 2;
hhhhhhh) I, V, M, F at a residue corresponding to position 273 in SEQ ID NO: 2;
iiiiiii) C, M, H, R, Y at a residue corresponding to position 274 in SEQ ID NO: 2;
jjjjjjj) H, C at a residue corresponding to position 275 in SEQ ID NO: 2;
kkkkkkk) A at a residue corresponding to position 277 in SEQ ID NO: 2;
lllllll) T, A, C at a residue corresponding to position 278 in SEQ ID NO: 2;
mmmmmmm) M, I, V at a residue corresponding to position 279 in SEQ ID NO: 2;
nnnnnnn) V, L at a residue corresponding to position 281 in SEQ ID NO: 2;
ooooooo) E, D, P, R at a residue corresponding to position 283 in SEQ ID NO: 2;
ppppppp) R, F, D at a residue corresponding to position 285 in SEQ ID NO: 2;
qqqqqqq) D, F at a residue corresponding to position 286 in SEQ ID NO: 2;
rrrrrrr) A, P, V at a residue corresponding to position 287 in SEQ ID NO: 2;
sssssss) A at a residue corresponding to position 290 in SEQ ID NO: 2;
ttttttt) M, I, L, V at a residue corresponding to position 291 in SEQ ID NO: 2;
uuuuuuu) H at a residue corresponding to position 292 in SEQ ID NO: 2;
vvvvvvv) A at a residue corresponding to position 294 in SEQ ID NO: 2;
wwwwwww) V, I, M at a residue corresponding to position 295 in SEQ ID NO: 2;
xxxxxxx) Q, D at a residue corresponding to position 296 in SEQ ID NO: 2;
yyyyyyy) M, V, L at a residue corresponding to position 298 in SEQ ID NO: 2;
zzzzzzz) A at a residue corresponding to position 299 in SEQ ID NO: 2;
aaaaaaaa) Q at a residue corresponding to position 300 in SEQ ID NO: 2;
bbbbbbbb) Q at a residue corresponding to position 302 in SEQ ID NO: 2;
cccccccc) A, P, C, V at a residue corresponding to position 303 in SEQ ID NO: 2;
dddddddd) S at a residue corresponding to position 304 in SEQ ID NO: 2;
eeeeeeee) V, M, I at a residue corresponding to position 305 in SEQ ID NO: 2;
ffffffff) I, V, Y at a residue corresponding to position 306 in SEQ ID NO: 2;
gggggggg) M, W, P at a residue corresponding to position 307 in SEQ ID NO: 2;
hhhhhhhh) G at a residue corresponding to position 308 in SEQ ID NO: 2;
iiiiiiii) T, A, R at a residue corresponding to position 311 in SEQ ID NO: 2;
jjjjjjjj) F, V, M at a residue corresponding to position 312 in SEQ ID NO: 2;
kkkkkkkk) F, W at a residue corresponding to position 313 in SEQ ID NO: 2;
llllllll) Q, H, E at a residue corresponding to position 314 in SEQ ID NO: 2;
mmmmmmmm) A, K, L, R at a residue corresponding to position 315 in SEQ ID NO: 2;
nnnnnnnn) I, M at a residue corresponding to position 316 in SEQ ID NO: 2;
oooooooo) D, H at a residue corresponding to position 318 in SEQ ID NO: 2;
pppppppp) R, Q at a residue corresponding to position 321 in SEQ ID NO: 2;
qqqqqqqq) A, K, H, Q at a residue corresponding to position 322 in SEQ ID NO: 2;
rrrrrrrr) F at a residue corresponding to position 330 in SEQ ID NO: 2;
ssssssss) G at a residue corresponding to position 331 in SEQ ID NO: 2;
tttttttt) S at a residue corresponding to position 333 in SEQ ID NO: 2;
uuuuuuuu) V, I, M at a residue corresponding to position 334 in SEQ ID NO: 2;
vvvvvvvv) K at a residue corresponding to position 335 in SEQ ID NO: 2;
wwwwwwww) V, T, M, Y, W, I, F at a residue corresponding to position 336 in SEQ ID NO: 2;
xxxxxxxx) L, V, M at a residue corresponding to position 337 in SEQ ID NO: 2;
yyyyyyyy) L, M, V, W at a residue corresponding to position 338 in SEQ ID NO: 2;
zzzzzzzz) T, S, F at a residue corresponding to position 339 in SEQ ID NO: 2;
aaaaaaaaa) G at a residue corresponding to position 340 in SEQ ID NO: 2;
bbbbbbbbb) S at a residue corresponding to position 342 in SEQ ID NO: 2;
ccccccccc) P, A at a residue corresponding to position 343 in SEQ ID NO: 2;
ddddddddd) I, C, M, V at a residue corresponding to position 344 in SEQ ID NO: 2;
eeeeeeeee) S at a residue corresponding to position 345 in SEQ ID NO: 2;
fffffffff) E, V, A, P at a residue corresponding to position 346 in SEQ ID NO: 2;
ggggggggg) E at a residue corresponding to position 347 in SEQ ID NO: 2;
hhhhhhhhh) I, L, T at a residue corresponding to position 348 in SEQ ID NO: 2;
iiiiiiiii) Y, F, L at a residue corresponding to position 349 in SEQ ID NO: 2;
jjjjjjjjj) R, Q, W at a residue corresponding to position 351 in SEQ ID NO: 2;
kkkkkkkkk) I, M at a residue corresponding to position 352 in SEQ ID NO: 2;
lllllllll) R at a residue corresponding to position 353 in SEQ ID NO: 2;
mmmmmmmmm) K at a residue corresponding to position 354 in SEQ ID NO: 2;
nnnnnnnnn) A at a residue corresponding to position 355 in SEQ ID NO: 2;
ooooooooo) F, I, V, A, G, L, W at a residue corresponding to position 357 in SEQ ID NO: 2;
ppppppppp) Q at a residue corresponding to position 358 in SEQ ID NO: 2;
qqqqqqqqq) Q at a residue corresponding to position 359 in SEQ ID NO: 2;
rrrrrrrrr) C, H, L at a residue corresponding to position 362 in SEQ ID NO: 2;
sssssssss) V, I, G at a residue corresponding to position 363 in SEQ ID NO: 2;
ttttttttt) M, I, V at a residue corresponding to position 365 in SEQ ID NO: 2;
uuuuuuuuu) I, L, F, V at a residue corresponding to position 366 in SEQ ID NO: 2;
vvvvvvvvv) V, I, L, M, T, C at a residue corresponding to position 367 in SEQ ID NO: 2;
wwwwwwwww) N at a residue corresponding to position 368 in SEQ ID NO: 2;
xxxxxxxxx) A, G at a residue corresponding to position 369 in SEQ ID NO: 2;
yyyyyyyyy) Y at a residue corresponding to position 370 in SEQ ID NO: 2;
zzzzzzzzz) C, A at a residue corresponding to position 375 in SEQ ID NO: 2;
aaaaaaaaaa) G, S at a residue corresponding to position 376 in SEQ ID NO: 2;
bbbbbbbbbb) G at a residue corresponding to position 377 in SEQ ID NO: 2;
cccccccccc) T at a residue corresponding to position 378 in SEQ ID NO: 2;
dddddddddd) V, C, L, A, I at a residue corresponding to position 379 in SEQ ID NO: 2;
eeeeeeeeee) S, V, H, T at a residue corresponding to position 380 in SEQ ID NO: 2;
ffffffffff) L, C at a residue corresponding to position 381 in SEQ ID NO: 2;
gggggggggg) T, C, N, L, P at a residue corresponding to position 382 in SEQ ID NO: 2;
hhhhhhhhhh) Y, R at a residue corresponding to position 383 in SEQ ID NO: 2;
iiiiiiiiii) N, W, D at a residue corresponding to position 384 in SEQ ID NO: 2;
jjjjjjjjjj) G at a residue corresponding to position 385 in SEQ ID NO: 2;
kkkkkkkkkk) V, T, L at a residue corresponding to position 386 in SEQ ID NO: 2;
llllllllll) R, H, N, D, E at a residue corresponding to position 387 in SEQ ID NO: 2;
mmmmmmmmmm) K, H at a residue corresponding to position 388 in SEQ ID NO: 2;
nnnnnnnnnn) A, P, L at a residue corresponding to position 389 in SEQ ID NO: 2;
oooooooooo) S, T at a residue corresponding to position 391 in SEQ ID NO: 2;
pppppppppp) V at a residue corresponding to position 392 in SEQ ID NO: 2;
qqqqqqqqqq) I, K at a residue corresponding to position 394 in SEQ ID NO: 2;
rrrrrrrrrr) V at a residue corresponding to position 396 in SEQ ID NO: 2;
ssssssssss) H at a residue corresponding to position 398 in SEQ ID NO: 2;
tttttttttt) C, I, T, V at a residue corresponding to position 399 in SEQ ID NO: 2;
uuuuuuuuuu) Q, D at a residue corresponding to position 400 in SEQ ID NO: 2;
vvvvvvvvvv) L, I, C, A, M at a residue corresponding to position 401 in SEQ ID NO: 2;
wwwwwwwwww) V at a residue corresponding to position 404 in SEQ ID NO: 2;
xxxxxxxxxx) E at a residue corresponding to position 406 in SEQ ID NO: 2;
yyyyyyyyyy) P at a residue corresponding to position 407 in SEQ ID NO: 2;
zzzzzzzzzz) G, Q, E at a residue corresponding to position 409 in SEQ ID NO: 2;
aaaaaaaaaaa) S at a residue corresponding to position 411 in SEQ ID NO: 2;
bbbbbbbbbbb) L, C at a residue corresponding to position 414 in SEQ ID NO: 2;
ccccccccccc) R at a residue corresponding to position 416 in SEQ ID NO: 2;
ddddddddddd) Q, H, M, S at a residue corresponding to position 419 in SEQ ID NO: 2;
eeeeeeeeeee) V at a residue corresponding to position 420 in SEQ ID NO: 2;
fffffffffff) F, T at a residue corresponding to position 421 in SEQ ID NO: 2;
ggggggggggg) L, H at a residue corresponding to position 425 in SEQ ID NO: 2;
hhhhhhhhhhh) A at a residue corresponding to position 430 in SEQ ID NO: 2;
iiiiiiiiiii) K at a residue corresponding to position 432 in SEQ ID NO: 2;
jjjjjjjjjjj) R, D at a residue corresponding to position 433 in SEQ ID NO: 2;
kkkkkkkkkkk) V, T at a residue corresponding to position 434 in SEQ ID NO: 2;
lllllllllll) M at a residue corresponding to position 435 in SEQ ID NO: 2;
mmmmmmmmmmm) F, M at a residue corresponding to position 441 in SEQ ID NO: 2;
nnnnnnnnnnn) Y, R, K, C, H at a residue corresponding to position 442 in SEQ ID NO: 2;
ooooooooooo) S at a residue corresponding to position 443 in SEQ ID NO: 2;
ppppppppppp) L, V, M, I at a residue corresponding to position 446 in SEQ ID NO: 2;
qqqqqqqqqqq) A at a residue corresponding to position 447 in SEQ ID NO: 2;
rrrrrrrrrrr) M, D at a residue corresponding to position 450 in SEQ ID NO: 2;
sssssssssss) E at a residue corresponding to position 451 in SEQ ID NO: 2;
ttttttttttt) P at a residue corresponding to position 452 in SEQ ID NO: 2;
uuuuuuuuuuu) G at a residue corresponding to position 453 in SEQ ID NO: 2;
vvvvvvvvvvv) N, D at a residue corresponding to position 456 in SEQ ID NO: 2;
wwwwwwwwwww) F, I, R, W, Y at a residue corresponding to position 460 in SEQ ID NO: 2;
xxxxxxxxxxx) Y, L at a residue corresponding to position 461 in SEQ ID NO: 2;
yyyyyyyyyyy) V, I at a residue corresponding to position 462 in SEQ ID NO: 2;
zzzzzzzzzzz) D at a residue corresponding to position 463 in SEQ ID NO: 2;
aaaaaaaaaaaa) L at a residue corresponding to position 465 in SEQ ID NO: 2;
bbbbbbbbbbbb) A, D, K at a residue corresponding to position 466 in SEQ ID NO: 2;
cccccccccccc) K at a residue corresponding to position 467 in SEQ ID NO: 2;
dddddddddddd) M at a residue corresponding to position 468 in SEQ ID NO: 2;
eeeeeeeeeeee) L at a residue corresponding to position 469 in SEQ ID NO: 2;
ffffffffffff) V at a residue corresponding to position 470 in SEQ ID NO: 2;
gggggggggggg) T, V at a residue corresponding to position 471 in SEQ ID NO: 2;
hhhhhhhhhhhh) N, S at a residue corresponding to position 473 in SEQ ID NO: 2;
iiiiiiiiiiii) Y, F, V, L, I, N at a residue corresponding to position 476 in SEQ ID NO: 2;
jjjjjjjjjjjj) T at a residue corresponding to position 478 in SEQ ID NO: 2;
kkkkkkkkkkkk) V, P at a residue corresponding to position 479 in SEQ ID NO: 2;
llllllllllll) S, A, P, L at a residue corresponding to position 480 in SEQ ID NO: 2;
mmmmmmmmmmmm) P, E at a residue corresponding to position 481 in SEQ ID NO: 2;
nnnnnnnnnnnn) L, E, R, A at a residue corresponding to position 485 in SEQ ID NO: 2;
oooooooooooo) C at a residue corresponding to position 490 in SEQ ID NO: 2;
pppppppppppp) P at a residue corresponding to position 492 in SEQ ID NO: 2;
qqqqqqqqqqqq) C at a residue corresponding to position 498 in SEQ ID NO: 2;
rrrrrrrrrrrr) V at a residue corresponding to position 507 in SEQ ID NO: 2;
ssssssssssss) V at a residue corresponding to position 508 in SEQ ID NO: 2;
tttttttttttt) A at a residue corresponding to position 510 in SEQ ID NO: 2;
uuuuuuuuuuuu) V, L at a residue corresponding to position 511 in SEQ ID NO: 2;
vvvvvvvvvvvv) I at a residue corresponding to position 512 in SEQ ID NO: 2;
wwwwwwwwwwww) N at a residue corresponding to position 514 in SEQ ID NO: 2;
xxxxxxxxxxxx) D, S at a residue corresponding to position 515 in SEQ ID NO: 2;
yyyyyyyyyyyy) A at a residue corresponding to position 516 in SEQ ID NO: 2;
zzzzzzzzzzzz) I at a residue corresponding to position 518 in SEQ ID NO: 2;
aaaaaaaaaaaaa) T at a residue corresponding to position 519 in SEQ ID NO: 2;
bbbbbbbbbbbbb) P, W at a residue corresponding to position 521 in SEQ ID NO: 2;
ccccccccccccc) M, L at a residue corresponding to position 526 in SEQ ID NO: 2;
ddddddddddddd) L at a residue corresponding to position 527 in SEQ ID NO: 2;
eeeeeeeeeeeee) R, H at a residue corresponding to position 532 in SEQ ID NO: 2;
fffffffffffff) L at a residue corresponding to position 535 in SEQ ID NO: 2;
ggggggggggggg) Q at a residue corresponding to position 540 in SEQ ID NO: 2;
hhhhhhhhhhhhh) Y, W at a residue corresponding to position 542 in SEQ ID NO: 2;
iiiiiiiiiiiii) N at a residue corresponding to position 546 in SEQ ID NO: 2;
jjjjjjjjjjjjj) Q at a residue corresponding to position 552 in SEQ ID NO: 2;
kkkkkkkkkkkkk) E at a residue corresponding to position 554 in SEQ ID NO: 2;
lllllllllllll) D at a residue corresponding to position 560 in SEQ ID NO: 2;
mmmmmmmmmmmmm) D at a residue corresponding to position 561 in SEQ ID NO: 2;
nnnnnnnnnnnnn) L, F at a residue corresponding to position 563 in SEQ ID NO: 2;
ooooooooooooo) S, T, L, D, R, K at a residue corresponding to position 564 in SEQ ID NO: 2;
ppppppppppppp) T, M, P, V, I at a residue corresponding to position 565 in SEQ ID NO: 2;
qqqqqqqqqqqqq) D, A, E, P at a residue corresponding to position 566 in SEQ ID NO: 2;
rrrrrrrrrrrrr) A, S, Q, N, T, K at a residue corresponding to position 567 in SEQ ID NO: 2;
sssssssssssss) G, D, N at a residue corresponding to position 568 in SEQ ID NO: 2;
ttttttttttttt) M, K at a residue corresponding to position 569 in SEQ ID NO: 2;
uuuuuuuuuuuuu) V, L at a residue corresponding to position 570 in SEQ ID NO: 2;
vvvvvvvvvvvvv) P at a residue corresponding to position 572 in SEQ ID NO: 2;
wwwwwwwwwwwww) S, T at a residue corresponding to position 573 in SEQ ID NO: 2;
xxxxxxxxxxxxx) M, L at a residue corresponding to position 574 in SEQ ID NO: 2;
yyyyyyyyyyyyy) T, Y at a residue corresponding to position 575 in SEQ ID NO: 2;
zzzzzzzzzzzzz) A, L at a residue corresponding to position 577 in SEQ ID NO: 2;
aaaaaaaaaaaaaa) A, Q at a residue corresponding to position 578 in SEQ ID NO: 2;
bbbbbbbbbbbbbb) D at a residue corresponding to position 583 in SEQ ID NO: 2;
cccccccccccccc) N at a residue corresponding to position 584 in SEQ ID NO: 2;
dddddddddddddd) H at a residue corresponding to position 585 in SEQ ID NO: 2;
eeeeeeeeeeeeee) A at a residue corresponding to position 587 in SEQ ID NO: 2;
ffffffffffffff) Q at a residue corresponding to position 588 in SEQ ID NO: 2;
gggggggggggggg) Q, E at a residue corresponding to position 591 in SEQ ID NO: 2;
hhhhhhhhhhhhhh) A at a residue corresponding to position 593 in SEQ ID NO: 2;
iiiiiiiiiiiiii) E at a residue corresponding to position 595 in SEQ ID NO: 2;
jjjjjjjjjjjjjj) K at a residue corresponding to position 596 in SEQ ID NO: 2;
kkkkkkkkkkkkkk) D at a residue corresponding to position 598 in SEQ ID NO: 2;
llllllllllllll) S at a residue corresponding to position 599 in SEQ ID NO: 2;
mmmmmmmmmmmmmm) L at a residue corresponding to position 601 in SEQ ID NO: 2; or
nnnnnnnnnnnnnn) Any combination thereof.

27. The engineered ACL of claim 25 or 26, wherein the one or more amino acid alterations result in an engineered ACL comprising:
a) E, R at a residue corresponding to position 5 in SEQ ID NO: 2;
b) L at a residue corresponding to position 6 in SEQ ID NO: 2;
c) K, T at a residue corresponding to position 7 in SEQ ID NO: 2;
d) P at a residue corresponding to position 8 in SEQ ID NO: 2;
e) D, N, R, P at a residue corresponding to position 10 in SEQ ID NO: 2;
f) A, V, M at a residue corresponding to position 12 in SEQ ID NO: 2;
g) Q, P at a residue corresponding to position 14 in SEQ ID NO: 2;
h) S, E at a residue corresponding to position 15 in SEQ ID NO: 2;
i) I at a residue corresponding to position 16 in SEQ ID NO: 2;
j) D, R at a residue corresponding to position 19 in SEQ ID NO: 2;
k) H, K, R, D at a residue corresponding to position 20 in SEQ ID NO: 2;
l) E, D at a residue corresponding to position 22 in SEQ ID NO: 2;
m) M at a residue corresponding to position 28 in SEQ ID NO: 2;
n) R at a residue corresponding to position 39 in SEQ ID NO: 2;
o) V at a residue corresponding to position 41 in SEQ ID NO: 2;
p) S at a residue corresponding to position 43 in SEQ ID NO: 2;
q) I at a residue corresponding to position 45 in SEQ ID NO: 2;
r) R, L, I, V, F at a residue corresponding to position 50 in SEQ ID NO: 2;
s) F at a residue corresponding to position 57 in SEQ ID NO: 2;
t) F, M, L, V at a residue corresponding to position 58 in SEQ ID NO: 2;
u) P at a residue corresponding to position 61 in SEQ ID NO: 2;
v) D at a residue corresponding to position 62 in SEQ ID NO: 2;
w) D at a residue corresponding to position 63 in SEQ ID NO: 2;
x) K at a residue corresponding to position 67 in SEQ ID NO: 2;
y) F, L, Y at a residue corresponding to position 69 in SEQ ID NO: 2;
z) W, F at a residue corresponding to position 71 in SEQ ID NO: 2;
aa) A, R at a residue corresponding to position 72 in SEQ ID NO: 2;
bb) D, T, A, R at a residue corresponding to position 76 in SEQ ID NO: 2;
cc) R at a residue corresponding to position 77 in SEQ ID NO: 2;
dd) A at a residue corresponding to position 78 in SEQ ID NO: 2;
ee) C at a residue corresponding to position 81 in SEQ ID NO: 2;
ff) Y at a residue corresponding to position 84 in SEQ ID NO: 2;
gg) F at a residue corresponding to position 85 in SEQ ID NO: 2;
hh) Q at a residue corresponding to position 86 in SEQ ID NO: 2;
ii) R at a residue corresponding to position 87 in SEQ ID NO: 2;
jj) K at a residue corresponding to position 92 in SEQ ID NO: 2;
kk) G at a residue corresponding to position 93 in SEQ ID NO: 2;
ll) R at a residue corresponding to position 94 in SEQ ID NO: 2;
mm) R at a residue corresponding to position 95 in SEQ ID NO: 2;
nn) M at a residue corresponding to position 98 in SEQ ID NO: 2;
oo) A at a residue corresponding to position 104 in SEQ ID NO: 2;
pp) V at a residue corresponding to position 108 in SEQ ID NO: 2;
qq) V at a residue corresponding to position 109 in SEQ ID NO: 2;
rr) C at a residue corresponding to position 113 in SEQ ID NO: 2;
ss) I at a residue corresponding to position 116 in SEQ ID NO: 2;
tt) T at a residue corresponding to position 119 in SEQ ID NO: 2;
uu) Y, V, F, L, M, A at a residue corresponding to position 120 in SEQ ID NO: 2;
vv) I at a residue corresponding to position 123 in SEQ ID NO: 2;
ww) S, N at a residue corresponding to position 124 in SEQ ID NO: 2;
xx) P, T, W at a residue corresponding to position 125 in SEQ ID NO: 2;
yy) R, A, N at a residue corresponding to position 126 in SEQ ID NO: 2;
zz) L, W at a residue corresponding to position 127 in SEQ ID NO: 2;
aaa) M at a residue corresponding to position 130 in SEQ ID NO: 2;
bbb) E at a residue corresponding to position 131 in SEQ ID NO: 2;
ccc) E, K at a residue corresponding to position 136 in SEQ ID NO: 2;
ddd) M, I, C at a residue corresponding to position 140 in SEQ ID NO: 2;
eee) M at a residue corresponding to position 141 in SEQ ID NO: 2;
fff) Q, N at a residue corresponding to position 142 in SEQ ID NO: 2;
ggg) R at a residue corresponding to position 145 in SEQ ID NO: 2;
hhh) S at a residue corresponding to position 147 in SEQ ID NO: 2;
iii) V at a residue corresponding to position 148 in SEQ ID NO: 2;
jjj) V at a residue corresponding to position 149 in SEQ ID NO: 2;
kkk) C at a residue corresponding to position 151 in SEQ ID NO: 2;
lll) E, R at a residue corresponding to position 159 in SEQ ID NO: 2;
mmm) I at a residue corresponding to position 161 in SEQ ID NO: 2;
nnn) V at a residue corresponding to position 163 in SEQ ID NO: 2;
ooo) A at a residue corresponding to position 164 in SEQ ID NO: 2;
ppp) R at a residue corresponding to position 167 in SEQ ID NO: 2;
qqq) H at a residue corresponding to position 168 in SEQ ID NO: 2;
rrr) I at a residue corresponding to position 169 in SEQ ID NO: 2;
sss) I, L, V at a residue corresponding to position 170 in SEQ ID NO: 2;
ttt) V at a residue corresponding to position 173 in SEQ ID NO: 2;
uuu) R at a residue corresponding to position 175 in SEQ ID NO: 2;
vvv) D at a residue corresponding to position 176 in SEQ ID NO: 2;
www) E at a residue corresponding to position 177 in SEQ ID NO: 2;
xxx) E at a residue corresponding to position 181 in SEQ ID NO: 2;
yyy) I at a residue corresponding to position 183 in SEQ ID NO: 2;
zzz) S at a residue corresponding to position 185 in SEQ ID NO: 2;
aaaa) Y at a residue corresponding to position 186 in SEQ ID NO: 2;
bbbb) D at a residue corresponding to position 187 in SEQ ID NO: 2;
cccc) V at a residue corresponding to position 190 in SEQ ID NO: 2;
dddd) E, A at a residue corresponding to position 191 in SEQ ID NO: 2;
eeee) A at a residue corresponding to position 193 in SEQ ID NO: 2;
ffff) S at a residue corresponding to position 194 in SEQ ID NO: 2;
gggg) S at a residue corresponding to position 195 in SEQ ID NO: 2;
hhhh) L, A at a residue corresponding to position 201 in SEQ ID NO: 2;
iiii) T at a residue corresponding to position 205 in SEQ ID NO: 2;
jjjj) I at a residue corresponding to position 209 in SEQ ID NO: 2;
kkkk) Y at a residue corresponding to position 214 in SEQ ID NO: 2;
llll) T at a residue corresponding to position 218 in SEQ ID NO: 2;
mmmm) T at a residue corresponding to position 219 in SEQ ID NO: 2;
nnnn) G at a residue corresponding to position 220 in SEQ ID NO: 2;
oooo) K, L, T, H at a residue corresponding to position 221 in SEQ ID NO: 2;
pppp) M, V at a residue corresponding to position 226 in SEQ ID NO: 2;
qqqq) S at a residue corresponding to position 228 in SEQ ID NO: 2;
rrrr) H, K at a residue corresponding to position 230 in SEQ ID NO: 2;
ssss) L at a residue corresponding to position 232 in SEQ ID NO: 2;
tttt) S at a residue corresponding to position 234 in SEQ ID NO: 2;
uuuu) A at a residue corresponding to position 237 in SEQ ID NO: 2;
vvvv) M at a residue corresponding to position 238 in SEQ ID NO: 2;
wwww) V at a residue corresponding to position 239 in SEQ ID NO: 2;
xxxx) Q, E at a residue corresponding to position 241 in SEQ ID NO: 2;
yyyy) C, V, T at a residue corresponding to position 242 in SEQ ID NO: 2;
zzzz) W, F, M at a residue corresponding to position 243 in SEQ ID NO: 2;
aaaaa) P, Q at a residue corresponding to position 244 in SEQ ID NO: 2;
bbbbb) I at a residue corresponding to position 246 in SEQ ID NO: 2;
ccccc) R at a residue corresponding to position 247 in SEQ ID NO: 2;
ddddd) L, V at a residue corresponding to position 253 in SEQ ID NO: 2;
eeeee) M, I, V at a residue corresponding to position 254 in SEQ ID NO: 2;
fffff) S, H at a residue corresponding to position 260 in SEQ ID NO: 2;
ggggg) T at a residue corresponding to position 262 in SEQ ID NO: 2;
hhhhh) Y at a residue corresponding to position 263 in SEQ ID NO: 2;
iiiii) N at a residue corresponding to position 266 in SEQ ID NO: 2;
jjjjj) M, F at a residue corresponding to position 267 in SEQ ID NO: 2;
kkkkk) A at a residue corresponding to position 272 in SEQ ID NO: 2;
lllll) I, V, M, F at a residue corresponding to position 273 in SEQ ID NO: 2;
mmmmm) C at a residue corresponding to position 274 in SEQ ID NO: 2;
nnnnn) T, A at a residue corresponding to position 278 in SEQ ID NO: 2;
ooooo) M at a residue corresponding to position 279 in SEQ ID NO: 2;
ppppp) V at a residue corresponding to position 281 in SEQ ID NO: 2;
qqqqq) E at a residue corresponding to position 283 in SEQ ID NO: 2;
rrrrr) R at a residue corresponding to position 285 in SEQ ID NO: 2;
sssss) A at a residue corresponding to position 290 in SEQ ID NO: 2;
ttttt) M, I, L, V at a residue corresponding to position 291 in SEQ ID NO: 2;
uuuuu) H at a residue corresponding to position 292 in SEQ ID NO: 2;
vvvvv) V, I, M at a residue corresponding to position 295 in SEQ ID NO: 2;
wwwww) Q at a residue corresponding to position 296 in SEQ ID NO: 2;
xxxxx) M, V at a residue corresponding to position 298 in SEQ ID NO: 2;
yyyyy) Q at a residue corresponding to position 300 in SEQ ID NO: 2;
zzzzz) Q at a residue corresponding to position 302 in SEQ ID NO: 2;
aaaaaa) A, P at a residue corresponding to position 303 in SEQ ID NO: 2;
bbbbbb) S at a residue corresponding to position 304 in SEQ ID NO: 2;
cccccc) V, M, I at a residue corresponding to position 305 in SEQ ID NO: 2;
dddddd) M at a residue corresponding to position 307 in SEQ ID NO: 2;
eeeeee) T, A at a residue corresponding to position 311 in SEQ ID NO: 2;
ffffff) F at a residue corresponding to position 313 in SEQ ID NO: 2;
gggggg) Q, H at a residue corresponding to position 314 in SEQ ID NO: 2;
hhhhhh) A, K, L, R at a residue corresponding to position 315 in SEQ ID NO: 2;
iiiiii) I at a residue corresponding to position 316 in SEQ ID NO: 2;
jjjjjj) D, H at a residue corresponding to position 318 in SEQ ID NO: 2;
kkkkkk) R, Q at a residue corresponding to position 321 in SEQ ID NO: 2;
llllll) A, K, H, Q at a residue corresponding to position 322 in SEQ ID NO: 2;
mmmmmm) F at a residue corresponding to position 330 in SEQ ID NO: 2;
nnnnnn) S at a residue corresponding to position 333 in SEQ ID NO: 2;
oooooo) V, I at a residue corresponding to position 334 in SEQ ID NO: 2;
pppppp) V, T, M, Y at a residue corresponding to position 336 in SEQ ID NO: 2;
qqqqqq) L, V at a residue corresponding to position 337 in SEQ ID NO: 2;
rrrrrr) L, M, V at a residue corresponding to position 338 in SEQ ID NO: 2;
ssssss) T at a residue corresponding to position 339 in SEQ ID NO: 2;
tttttt) G at a residue corresponding to position 340 in SEQ ID NO: 2;
uuuuuu) I, C, M at a residue corresponding to position 344 in SEQ ID NO: 2;
vvvvvv) S at a residue corresponding to position 345 in SEQ ID NO: 2;
wwwwww) E at a residue corresponding to position 346 in SEQ ID NO: 2;
xxxxxx) E at a residue corresponding to position 347 in SEQ ID NO: 2;
yyyyyy) I, L, T at a residue corresponding to position 348 in SEQ ID NO: 2;
zzzzzz) Y, F at a residue corresponding to position 349 in SEQ ID NO: 2;
aaaaaaa) R, Q at a residue corresponding to position 351 in SEQ ID NO: 2;
bbbbbbb) I at a residue corresponding to position 352 in SEQ ID NO: 2;
ccccccc) R at a residue corresponding to position 353 in SEQ ID NO: 2;
ddddddd) K at a residue corresponding to position 354 in SEQ ID NO: 2;
eeeeeee) A at a residue corresponding to position 355 in SEQ ID NO: 2;
fffffff) F, I, V, A, G, L, W at a residue corresponding to position 357 in SEQ ID NO: 2;
ggggggg) Q at a residue corresponding to position 358 in SEQ ID NO: 2;
hhhhhhh) Q at a residue corresponding to position 359 in SEQ ID NO: 2;
iiiiiii) C, H at a residue corresponding to position 362 in SEQ ID NO: 2;
jjjjjjj) V, I at a residue corresponding to position 363 in SEQ ID NO: 2;
kkkkkkk) M at a residue corresponding to position 365 in SEQ ID NO: 2;
lllllll) V, I, L, M at a residue corresponding to position 367 in SEQ ID NO: 2;
mmmmmmm) A at a residue corresponding to position 369 in SEQ ID NO: 2;
nnnnnnn) C at a residue corresponding to position 375 in SEQ ID NO: 2;
ooooooo) G, S at a residue corresponding to position 376 in SEQ ID NO: 2;
ppppppp) V, C, L at a residue corresponding to position 379 in SEQ ID NO: 2;
qqqqqqq) T, C at a residue corresponding to position 382 in SEQ ID NO: 2;
rrrrrrr) Y at a residue corresponding to position 383 in SEQ ID NO: 2;
sssssss) N at a residue corresponding to position 384 in SEQ ID NO: 2;
ttttttt) V, T at a residue corresponding to position 386 in SEQ ID NO: 2;
uuuuuuu) R, H, N, D at a residue corresponding to position 387 in SEQ ID NO: 2;
vvvvvvv) K, H at a residue corresponding to position 388 in SEQ ID NO: 2;
wwwwwww) A, P, L at a residue corresponding to position 389 in SEQ ID NO: 2;
xxxxxxx) C, I, T, V at a residue corresponding to position 399 in SEQ ID NO: 2;
yyyyyyy) Q at a residue corresponding to position 400 in SEQ ID NO: 2;
zzzzzzz) L, I, C at a residue corresponding to position 401 in SEQ ID NO: 2;
aaaaaaaa) V at a residue corresponding to position 404 in SEQ ID NO: 2;
bbbbbbbb) P at a residue corresponding to position 407 in SEQ ID NO: 2;
cccccccc) G at a residue corresponding to position 409 in SEQ ID NO: 2;
dddddddd) R at a residue corresponding to position 416 in SEQ ID NO: 2;
eeeeeeee) Q at a residue corresponding to position 419 in SEQ ID NO: 2;
ffffffff) V at a residue corresponding to position 420 in SEQ ID NO: 2;
gggggggg) F at a residue corresponding to position 421 in SEQ ID NO: 2;
hhhhhhhh) A at a residue corresponding to position 430 in SEQ ID NO: 2;
iiiiiiii) K at a residue corresponding to position 432 in SEQ ID NO: 2;
jjjjjjjj) R, D at a residue corresponding to position 433 in SEQ ID NO: 2;
kkkkkkkk) V at a residue corresponding to position 434 in SEQ ID NO: 2;
llllllll) F at a residue corresponding to position 441 in SEQ ID NO: 2;
mmmmmmmm) Y, R, K, C, H at a residue corresponding to position 442 in SEQ ID NO: 2;
nnnnnnnn) L, V, M, I at a residue corresponding to position 446 in SEQ ID NO: 2;
oooooooo) M at a residue corresponding to position 450 in SEQ ID NO: 2;
pppppppp) P at a residue corresponding to position 452 in SEQ ID NO: 2;
qqqqqqqq) G at a residue corresponding to position 453 in SEQ ID NO: 2;
rrrrrrrr) N, D at a residue corresponding to position 456 in SEQ ID NO: 2;
ssssssss) F, I, R at a residue corresponding to position 460 in SEQ ID NO: 2;
tttttttt) Y at a residue corresponding to position 461 in SEQ ID NO: 2;
uuuuuuuu) L at a residue corresponding to position 465 in SEQ ID NO: 2;
vvvvvvvv) A, D at a residue corresponding to position 466 in SEQ ID NO: 2;
wwwwwwww) T at a residue corresponding to position 471 in SEQ ID NO: 2;
xxxxxxxx) N, S at a residue corresponding to position 473 in SEQ ID NO: 2;
yyyyyyyy) Y, F, V, L, I at a residue corresponding to position 476 in SEQ ID NO: 2;
zzzzzzzz) V, P at a residue corresponding to position 479 in SEQ ID NO: 2;
aaaaaaaaa) S, A, P at a residue corresponding to position 480 in SEQ ID NO: 2;
bbbbbbbbb) P, E at a residue corresponding to position 481 in SEQ ID NO: 2;
ccccccccc) L, E, R, A at a residue corresponding to position 485 in SEQ ID NO: 2;
ddddddddd) C at a residue corresponding to position 490 in SEQ ID NO: 2;
eeeeeeeee) P at a residue corresponding to position 492 in SEQ ID NO: 2;
fffffffff) V at a residue corresponding to position 507 in SEQ ID NO: 2;
ggggggggg) V at a residue corresponding to position 511 in SEQ ID NO: 2;
hhhhhhhhh) N at a residue corresponding to position 514 in SEQ ID NO: 2;
iiiiiiiii) D, S at a residue corresponding to position 515 in SEQ ID NO: 2;
jjjjjjjjj) A at a residue corresponding to position 516 in SEQ ID NO: 2;
kkkkkkkkk) I at a residue corresponding to position 518 in SEQ ID NO: 2;
lllllllll) T at a residue corresponding to position 519 in SEQ ID NO: 2;
mmmmmmmmm) P, W at a residue corresponding to position 521 in SEQ ID NO: 2;
nnnnnnnnn) M, L at a residue corresponding to position 526 in SEQ ID NO: 2;
ooooooooo) L at a residue corresponding to position 527 in SEQ ID NO: 2;
ppppppppp) R, H at a residue corresponding to position 532 in SEQ ID NO: 2;
qqqqqqqqq) Q at a residue corresponding to position 540 in SEQ ID NO: 2;
rrrrrrrrr) Y at a residue corresponding to position 542 in SEQ ID NO: 2;
sssssssss) N at a residue corresponding to position 546 in SEQ ID NO: 2;
ttttttttt) E at a residue corresponding to position 554 in SEQ ID NO: 2;
uuuuuuuuu) D at a residue corresponding to position 561 in SEQ ID NO: 2;
vvvvvvvvv) S, T at a residue corresponding to position 564 in SEQ ID NO: 2;
wwwwwwwww) T, M, P, V, I at a residue corresponding to position 565 in SEQ ID NO: 2;
xxxxxxxxx) D at a residue corresponding to position 566 in SEQ ID NO: 2;
yyyyyyyyy) A, S, Q, N at a residue corresponding to position 567 in SEQ ID NO: 2;
zzzzzzzzz) G, D, N at a residue corresponding to position 568 in SEQ ID NO: 2;
aaaaaaaaaa) V at a residue corresponding to position 570 in SEQ ID NO: 2;
bbbbbbbbbb) P at a residue corresponding to position 572 in SEQ ID NO: 2;
cccccccccc) M at a residue corresponding to position 574 in SEQ ID NO: 2;
dddddddddd) T at a residue corresponding to position 575 in SEQ ID NO: 2;
eeeeeeeeee) A at a residue corresponding to position 577 in SEQ ID NO: 2;
ffffffffff) N at a residue corresponding to position 584 in SEQ ID NO: 2;
gggggggggg) A at a residue corresponding to position 587 in SEQ ID NO: 2;
hhhhhhhhhh) E at a residue corresponding to position 595 in SEQ ID NO: 2;
iiiiiiiiii) K at a residue corresponding to position 596 in SEQ ID NO: 2;
jjjjjjjjjj) S at a residue corresponding to position 599 in SEQ ID NO: 2; or
kkkkkkkkkk) Any combination thereof.

28. The engineered ACL of any one of claims 25 to 27, wherein the one or more amino acid alterations result in an engineered ACL comprising:
a) E, R at a residue corresponding to position 5 in SEQ ID NO: 2;
b) L at a residue corresponding to position 6 in SEQ ID NO: 2;
c) K, T at a residue corresponding to position 7 in SEQ ID NO: 2;
d) P at a residue corresponding to position 8 in SEQ ID NO: 2;
e) D, N at a residue corresponding to position 10 in SEQ ID NO: 2;
f) A at a residue corresponding to position 12 in SEQ ID NO: 2;
g) Q, P at a residue corresponding to position 14 in SEQ ID NO: 2;
h) S, E at a residue corresponding to position 15 in SEQ ID NO: 2;
i) D at a residue corresponding to position 19 in SEQ ID NO: 2;
j) H, K, R at a residue corresponding to position 20 in SEQ ID NO: 2;
k) E at a residue corresponding to position 22 in SEQ ID NO: 2;
l) V at a residue corresponding to position 41 in SEQ ID NO: 2;
m) I at a residue corresponding to position 45 in SEQ ID NO: 2;
n) R, L, I, V at a residue corresponding to position 50 in SEQ ID NO: 2;
o) F, M, L at a residue corresponding to position 58 in SEQ ID NO: 2;
p) D at a residue corresponding to position 62 in SEQ ID NO: 2;
q) D at a residue corresponding to position 63 in SEQ ID NO: 2;
r) K at a residue corresponding to position 67 in SEQ ID NO: 2;
s) F, L, Y at a residue corresponding to position 69 in SEQ ID NO: 2;
t) A, R at a residue corresponding to position 72 in SEQ ID NO: 2;
u) D, T, A, R at a residue corresponding to position 76 in SEQ ID NO: 2;
v) R at a residue corresponding to position 77 in SEQ ID NO: 2;
w) C at a residue corresponding to position 81 in SEQ ID NO: 2;
x) Q at a residue corresponding to position 86 in SEQ ID NO: 2;
y) R at a residue corresponding to position 87 in SEQ ID NO: 2;
z) K at a residue corresponding to position 92 in SEQ ID NO: 2;
aa) G at a residue corresponding to position 93 in SEQ ID NO: 2;
bb) R at a residue corresponding to position 94 in SEQ ID NO: 2;
cc) R at a residue corresponding to position 95 in SEQ ID NO: 2;
dd) M at a residue corresponding to position 98 in SEQ ID NO: 2;
ee) A at a residue corresponding to position 104 in SEQ ID NO: 2;
ff) V at a residue corresponding to position 108 in SEQ ID NO: 2;
gg) C at a residue corresponding to position 113 in SEQ ID NO: 2;
hh) I at a residue corresponding to position 116 in SEQ ID NO: 2;
ii) Y, V, F, L at a residue corresponding to position 120 in SEQ ID NO: 2;
jj) I at a residue corresponding to position 123 in SEQ ID NO: 2;
kk) S at a residue corresponding to position 124 in SEQ ID NO: 2;
ll) P, T at a residue corresponding to position 125 in SEQ ID NO: 2;
mm) R, A, N at a residue corresponding to position 126 in SEQ ID NO: 2;
nn) L at a residue corresponding to position 127 in SEQ ID NO: 2;
oo) M at a residue corresponding to position 130 in SEQ ID NO: 2;
pp) E at a residue corresponding to position 136 in SEQ ID NO: 2;
qq) M, I at a residue corresponding to position 140 in SEQ ID NO: 2;
rr) M at a residue corresponding to position 141 in SEQ ID NO: 2;
ss) Q, N at a residue corresponding to position 142 in SEQ ID NO: 2;
tt) R at a residue corresponding to position 145 in SEQ ID NO: 2;
uu) S at a residue corresponding to position 147 in SEQ ID NO: 2;
vv) V at a residue corresponding to position 148 in SEQ ID NO: 2;
ww) V at a residue corresponding to position 149 in SEQ ID NO: 2;
xx) C at a residue corresponding to position 151 in SEQ ID NO: 2;
yy) E at a residue corresponding to position 159 in SEQ ID NO: 2;
zz) I at a residue corresponding to position 161 in SEQ ID NO: 2;
aaa) V at a residue corresponding to position 163 in SEQ ID NO: 2;
bbb) A at a residue corresponding to position 164 in SEQ ID NO: 2;
ccc) R at a residue corresponding to position 167 in SEQ ID NO: 2;
ddd) H at a residue corresponding to position 168 in SEQ ID NO: 2;
eee) I at a residue corresponding to position 169 in SEQ ID NO: 2;
fff) R at a residue corresponding to position 175 in SEQ ID NO: 2;
ggg) D at a residue corresponding to position 176 in SEQ ID NO: 2;
hhh) E at a residue corresponding to position 177 in SEQ ID NO: 2;
iii) S at a residue corresponding to position 185 in SEQ ID NO: 2;
jjj) Y at a residue corresponding to position 186 in SEQ ID NO: 2;
kkk) D at a residue corresponding to position 187 in SEQ ID NO: 2;
lll) V at a residue corresponding to position 190 in SEQ ID NO: 2;
mmm) E, A at a residue corresponding to position 191 in SEQ ID NO: 2;
nnn) A at a residue corresponding to position 193 in SEQ ID NO: 2;
ooo) S at a residue corresponding to position 195 in SEQ ID NO: 2;
ppp) L, A at a residue corresponding to position 201 in SEQ ID NO: 2;
qqq) T at a residue corresponding to position 205 in SEQ ID NO: 2;
rrr) I at a residue corresponding to position 209 in SEQ ID NO: 2;
sss) Y at a residue corresponding to position 214 in SEQ ID NO: 2;
ttt) T at a residue corresponding to position 219 in SEQ ID NO: 2;
uuu) G at a residue corresponding to position 220 in SEQ ID NO: 2;
vvv) K at a residue corresponding to position 221 in SEQ ID NO: 2;
www) S at a residue corresponding to position 228 in SEQ ID NO: 2;
xxx) H, K at a residue corresponding to position 230 in SEQ ID NO: 2;
yyy) L at a residue corresponding to position 232 in SEQ ID NO: 2;
zzz) S at a residue corresponding to position 234 in SEQ ID NO: 2;
aaaa) A at a residue corresponding to position 237 in SEQ ID NO: 2;
bbbb) M at a residue corresponding to position 238 in SEQ ID NO: 2;
cccc) V at a residue corresponding to position 239 in SEQ ID NO: 2;
dddd) Q, E at a residue corresponding to position 241 in SEQ ID NO: 2;
eeee) C, V, T at a residue corresponding to position 242 in SEQ ID NO: 2;
ffff) W, F, M at a residue corresponding to position 243 in SEQ ID NO: 2;
gggg) P, Q at a residue corresponding to position 244 in SEQ ID NO: 2;
hhhh) I at a residue corresponding to position 246 in SEQ ID NO: 2;
iiii) R at a residue corresponding to position 247 in SEQ ID NO: 2;
jjjj) L at a residue corresponding to position 253 in SEQ ID NO: 2;
kkkk) M at a residue corresponding to position 254 in SEQ ID NO: 2;
llll) S, H at a residue corresponding to position 260 in SEQ ID NO: 2;
mmmm) Y at a residue corresponding to position 263 in SEQ ID NO: 2;
nnnn) N at a residue corresponding to position 266 in SEQ ID NO: 2;
oooo) M, F at a residue corresponding to position 267 in SEQ ID NO: 2;
pppp) A at a residue corresponding to position 272 in SEQ ID NO: 2;
qqqq) I at a residue corresponding to position 273 in SEQ ID NO: 2;
rrrr) C at a residue corresponding to position 274 in SEQ ID NO: 2;
ssss) T, A at a residue corresponding to position 278 in SEQ ID NO: 2;
tttt) M at a residue corresponding to position 279 in SEQ ID NO: 2;
uuuu) V at a residue corresponding to position 281 in SEQ ID NO: 2;
vvvv) E at a residue corresponding to position 283 in SEQ ID NO: 2;
wwww) M, I, L, V at a residue corresponding to position 291 in SEQ ID NO: 2;
xxxx) H at a residue corresponding to position 292 in SEQ ID NO: 2;
yyyy) V, I, M at a residue corresponding to position 295 in SEQ ID NO: 2;
zzzz) M, V at a residue corresponding to position 298 in SEQ ID NO: 2;
aaaaa) Q at a residue corresponding to position 300 in SEQ ID NO: 2;
bbbbb) Q at a residue corresponding to position 302 in SEQ ID NO: 2;
ccccc) A, P at a residue corresponding to position 303 in SEQ ID NO: 2;
ddddd) S at a residue corresponding to position 304 in SEQ ID NO: 2;
eeeee) V at a residue corresponding to position 305 in SEQ ID NO: 2;
fffff) M at a residue corresponding to position 307 in SEQ ID NO: 2;
ggggg) T, A at a residue corresponding to position 311 in SEQ ID NO: 2;
hhhhh) F at a residue corresponding to position 313 in SEQ ID NO: 2;
iiiii) Q, H at a residue corresponding to position 314 in SEQ ID NO: 2;
jjjjj) A, K at a residue corresponding to position 315 in SEQ ID NO: 2;
kkkkk) D at a residue corresponding to position 318 in SEQ ID NO: 2;
lllll) R, Q at a residue corresponding to position 321 in SEQ ID NO: 2;
mmmmm) A, K, H, Q at a residue corresponding to position 322 in SEQ ID NO: 2;
nnnnn) F at a residue corresponding to position 330 in SEQ ID NO: 2;
ooooo) S at a residue corresponding to position 333 in SEQ ID NO: 2;
ppppp) V, I at a residue corresponding to position 334 in SEQ ID NO: 2;
qqqqq) V, T, M at a residue corresponding to position 336 in SEQ ID NO: 2;
rrrrr) L, V at a residue corresponding to position 337 in SEQ ID NO: 2;
sssss) L, M at a residue corresponding to position 338 in SEQ ID NO: 2;
ttttt) G at a residue corresponding to position 340 in SEQ ID NO: 2;
uuuuu) I at a residue corresponding to position 344 in SEQ ID NO: 2;
vvvvv) I, L at a residue corresponding to position 348 in SEQ ID NO: 2;
wwwww) Y at a residue corresponding to position 349 in SEQ ID NO: 2;
xxxxx) R, Q at a residue corresponding to position 351 in SEQ ID NO: 2;
yyyyy) R at a residue corresponding to position 353 in SEQ ID NO: 2;
zzzzz) K at a residue corresponding to position 354 in SEQ ID NO: 2;
aaaaaa) A at a residue corresponding to position 355 in SEQ ID NO: 2;
bbbbbb) F, I at a residue corresponding to position 357 in SEQ ID NO: 2;
cccccc) Q at a residue corresponding to position 358 in SEQ ID NO: 2;
dddddd) Q at a residue corresponding to position 359 in SEQ ID NO: 2;
eeeeee) C, H at a residue corresponding to position 362 in SEQ ID NO: 2;
ffffff) V at a residue corresponding to position 363 in SEQ ID NO: 2;
gggggg) M at a residue corresponding to position 365 in SEQ ID NO: 2;
hhhhhh) V, I, L, M at a residue corresponding to position 367 in SEQ ID NO: 2;
iiiiii) A at a residue corresponding to position 369 in SEQ ID NO: 2;
jjjjjj) G at a residue corresponding to position 376 in SEQ ID NO: 2;
kkkkkk) V, C at a residue corresponding to position 379 in SEQ ID NO: 2;
llllll) T, C at a residue corresponding to position 382 in SEQ ID NO: 2;
mmmmmm) Y at a residue corresponding to position 383 in SEQ ID NO: 2;
nnnnnn) V, T at a residue corresponding to position 386 in SEQ ID NO: 2;
oooooo) R, H, N at a residue corresponding to position 387 in SEQ ID NO: 2;
pppppp) K at a residue corresponding to position 388 in SEQ ID NO: 2;
qqqqqq) A, P at a residue corresponding to position 389 in SEQ ID NO: 2;
rrrrrr) C, I, T, V at a residue corresponding to position 399 in SEQ ID NO: 2;
ssssss) Q at a residue corresponding to position 400 in SEQ ID NO: 2;
tttttt) L, I at a residue corresponding to position 401 in SEQ ID NO: 2;
uuuuuu) V at a residue corresponding to position 404 in SEQ ID NO: 2;
vvvvvv) P at a residue corresponding to position 407 in SEQ ID NO: 2;
wwwwww) G at a residue corresponding to position 409 in SEQ ID NO: 2;
xxxxxx) V at a residue corresponding to position 420 in SEQ ID NO: 2;
yyyyyy) F at a residue corresponding to position 421 in SEQ ID NO: 2;
zzzzzz) A at a residue corresponding to position 430 in SEQ ID NO: 2;
aaaaaaa) K at a residue corresponding to position 432 in SEQ ID NO: 2;
bbbbbbb) R, D at a residue corresponding to position 433 in SEQ ID NO: 2;
ccccccc) V at a residue corresponding to position 434 in SEQ ID NO: 2;
ddddddd) Y, R, K, C, H at a residue corresponding to position 442 in SEQ ID NO: 2;
eeeeeee) L, V, M, I at a residue corresponding to position 446 in SEQ ID NO: 2;
fffffff) M at a residue corresponding to position 450 in SEQ ID NO: 2;
ggggggg) P at a residue corresponding to position 452 in SEQ ID NO: 2;
hhhhhhh) G at a residue corresponding to position 453 in SEQ ID NO: 2;
iiiiiii) N, D at a residue corresponding to position 456 in SEQ ID NO: 2;
jjjjjjj) F, I at a residue corresponding to position 460 in SEQ ID NO: 2;
kkkkkkk) Y at a residue corresponding to position 461 in SEQ ID NO: 2;
lllllll) L at a residue corresponding to position 465 in SEQ ID NO: 2;
mmmmmmm) A at a residue corresponding to position 466 in SEQ ID NO: 2;
nnnnnnn) N, S at a residue corresponding to position 473 in SEQ ID NO: 2;
ooooooo) Y, F, V, L, I at a residue corresponding to position 476 in SEQ ID NO: 2;
ppppppp) V, P at a residue corresponding to position 479 in SEQ ID NO: 2;
qqqqqqq) S, A at a residue corresponding to position 480 in SEQ ID NO: 2;
rrrrrrr) P, E at a residue corresponding to position 481 in SEQ ID NO: 2;
sssssss) L, E, R, A at a residue corresponding to position 485 in SEQ ID NO: 2;
ttttttt) P at a residue corresponding to position 492 in SEQ ID NO: 2;
uuuuuuu) V at a residue corresponding to position 507 in SEQ ID NO: 2;
vvvvvvv) N at a residue corresponding to position 514 in SEQ ID NO: 2;
wwwwwww) D, S at a residue corresponding to position 515 in SEQ ID NO: 2;
xxxxxxx) A at a residue corresponding to position 516 in SEQ ID NO: 2;
yyyyyyy) I at a residue corresponding to position 518 in SEQ ID NO: 2;
zzzzzzz) T at a residue corresponding to position 519 in SEQ ID NO: 2;
aaaaaaaa) P at a residue corresponding to position 521 in SEQ ID NO: 2;
bbbbbbbb) M, L at a residue corresponding to position 526 in SEQ ID NO: 2;
cccccccc) L at a residue corresponding to position 527 in SEQ ID NO: 2;
dddddddd) R, H at a residue corresponding to position 532 in SEQ ID NO: 2;
eeeeeeee) Q at a residue corresponding to position 540 in SEQ ID NO: 2;
ffffffff) Y at a residue corresponding to position 542 in SEQ ID NO: 2;
gggggggg) E at a residue corresponding to position 554 in SEQ ID NO: 2;
hhhhhhhh) S, T at a residue corresponding to position 564 in SEQ ID NO: 2;
iiiiiiii) T, M, P, V, I at a residue corresponding to position 565 in SEQ ID NO: 2;
jjjjjjjj) A, S, Q, N at a residue corresponding to position 567 in SEQ ID NO: 2;
kkkkkkkk) G, D at a residue corresponding to position 568 in SEQ ID NO: 2;
llllllll) V at a residue corresponding to position 570 in SEQ ID NO: 2;
mmmmmmmm) P at a residue corresponding to position 572 in SEQ ID NO: 2;
nnnnnnnn) T at a residue corresponding to position 575 in SEQ ID NO: 2;
oooooooo) N at a residue corresponding to position 584 in SEQ ID NO: 2;
pppppppp) A at a residue corresponding to position 587 in SEQ ID NO: 2;
qqqqqqqq) E at a residue corresponding to position 595 in SEQ ID NO: 2;
rrrrrrrr) K at a residue corresponding to position 596 in SEQ ID NO: 2;
or
ssssssss) any combination thereof.

29. The engineered ACL of any one of claims 1 to 7, wherein the engineered ACL comprises one or more amino acid alterations selected from the group consisting of: D5R, D5E, A6L, S7K, S7T, G8P, E10K, E10D, E10P, E10N, E10R, L12A, L12I, L12V, L12M, P13C, H14P, H14A, H14Q, D15E, D15L, D15S, D15R, V16C, V16L, V16I, E19D, E19R, S20H, S20K, S20R, S20D, P22D, P22E, S25N, L28M, V37C, P39R, N40T, T41M, T41L, T41V, T41I, A43S, W44M, L45I, L45V, W48P, K50I, K50F, K50R, K50V, K50L, E51R, P53L, A57F, I58V, I58L, I58M, I58F, A59V, R61P, R61E, A62D, G63D, W66G, R67K, H69F, H69Y, H69L, Y71F, Y71W, N72A, N72R, L74F, L75D, N76T, N76D, N76A, N76R, M77R, V78A, N80K, V81C, G83S, G83H, G83Q, G83N, A84Y, A84H, L85F, L86A, L86Q, A87R, R88L, A92K, D93G, S94R, P95R, V97A, V98M, M99L, S100C, G101P, G103R, V104L, V104W, V104A, H106W, H106F, A107V, A107P, A107I, A107L, A107W, A107F, I108V, L109V, S110M, S110F, S110Y, S110A, S110D, A113T, A113C, A113V, Q114M, Q114W, Q114L, Q114G, Q114A, Y115A, V116I, P119V, P119T, P119I, I120M, I120F, I120V, I120A, I120L, I120W, I120Y, V121T, P122A, L123M, L123V, L1231, A124Y, A124S, A124N, E125P, E125T, E125W, Q126M, Q126N, Q126R, Q126A, Y127F, Y127W, Y127L, S128R, I130M, P131E, R136E, R136Q, R136K, L137M, V140M, V140C, V140I, L141M, E142N, E142Q, K145G, K145R, K145D, A147S, M148L, M148V, M148A, A149V, A149L, F150V, V151C, Y157L, S159R, S159E, L161I, L163V, P164A, Q165R, E167R, G168H, V169I, E170I, E170V, E170L, A173V, K175R, T176D, D177E, V181E, V183I, A185S, F186Y, A187D, L189V, L190V, G191A, G191E, H192T, G193A, Q194S, G195S, G196P, H201A, H201P, H201L, G205T, T208D, L209I, L209T, L209P, L209V, K211L, K211T, K211C, K211M, K211V, K211A, I212L, I212M, L213I, L213C, L213V, L213Q, L213M, F214Y, T215S, S216G, S218T, S219T, S220G, D221T, D221M, D221L, D221R, D221K, D221H, P222S, G224A, V225A, L226M, L226I, L226V, T227L, T227D, T227H, T227I, T228S, R230H, R230K, M231N, M232L, M232I, M232V, C233L, C233V, C233M, C233W, C233A, C233I, A234S, A234W, N235Q, N235S, Q236A, Q236C, Q236I, Q236M, Q236V, T237A, T237M, T237Q, T237R, T237E, Q238M, Q238S, Q238W, Q238N, Q238H, I239F, I239M, I239V, I239C, I239L, S240A, S240R, D241A, D241H, D241E, D241Q, A242V, A242C, A242T, A242W, L243I, L243F, L243M, L243W, L243V, R244E, R244P, R244Q, R244H, R244G, F245M, F245L, F245H, F245W, F245Y, L246I, L246T, L246M, K247R, K247P, K247E, P251E, R252H, R252V, I253Y, I253M, I253F, I253L, I253V, L254I, L254F, L254V, L254M, D255A, D255T, D255N, D255L, D255I, D255G, D255H, D255C, D255V, D255S, W256F, W256V, W256I, W256C, W256Y, L257M, W259L, W259F, N260H, N260S, N260Y, N260F, V262C, V262T, V262A, V262I, V262M, F263A, F263Y, F263H, G264A, G265M, G265L, G265W, G265F, G265C, S266N, S266T, S266M, S266V, H267I, H267G, H267M, H267W, H267F, H267L, H267V, H267C, H267A, N268M, N268A, N268C, N268G, N268V, F269L, F269M, F269I, F269V, N270M, N270H, N270Y, N270T, M271A, M271P, M271C, M271T, M271L, M272C, M272V, M272P, M272A, M272T, L273M, L273V, L273F, L273I, A274Y, A274R, A274V, A274M, A274C, A274H, A274W, N275G, N275C, N275S, N275H, G277A, G277C, S278C, S278T, S278A, S278H, L279V, L279M, L279I, I281F, I281L, I281V, I281M, D282P, D282M, D282E, D282A, D282H, N283E, N283D, N283P, N283R, G284F, G284R, G284W, K285R, K285F, K285W, K285D, P286D, P286F, T287P, T287A, T287V, G290A, F291M, F291V, F291L, F291P, F291I, E292H, T294I, T294L, T294A, L295W, L295M, L295V, L295I, R296D, R296Q, I298M, I298V, I298L, K299A, E300Q, P302Q, G303V, G303I, G303A, G303P, G303C, T304S, L305W, L305Y, L305V, L305I, L305F, L305H, L305M, A306V, A306Y, A306I, A306M, A306L, A306F, F307P, F307W, F307Y, F307H, F307M, N308T, N308A, N308S, N308L, N308G, N308V, N308M, V311A, V311R, V311P, V311T, G312I, G312M, G312V, G312F, Y313L, Y313F, Y313W, A314Q, A314E, A314H, M315R, M315A, M315K, M315L, L316W, L316M, L316I, L316F, N318H, N318D, E321R, E321Q, R322Q, R322K, R322A, R322H, Y330L, Y330F, F331G, E333S, L334I, L334M, L334V, D335R, D335K, L336T, L336I, L336M, L336Y, L336V, L336F, L336W, I337L, I337M, I337F, I337A, I337V, F338W, F338L, F338M, F338V, Y339F, Y339T, Y339S, Y339C, A340G, G341A, G341S, A342S, S343P, S343A, L344M, L344I, L344V, L344C, P345S, Q346V, Q346A, Q346P, Q346E, D347E, V348T, V348I, V348L, W349M, W349F, W349L, W349A, W349I, W349Y, S351F, S351R, S351Q, S351W, L352I, L352M, L352Y, E353R, R354K, F355A, F355R, M357W, M357G, M357V, M357L, M357P, M357I, M357F, M357A, E358Q, E359H, E359Q, G361F, S362H, S362L, S362C, L363D, L363V, L363I, L363G, L365M, L365V, L365I, M366I, M366V, M366L, M366F, A367T, A367C, A367M, A367I, A367V, A367L, S368N, S368Q, S368E, S368T, S369G, S369A, W370Y, W370F, M372L, M372Q, T373S, T375A, T375C, A376T, A376S, A376C, A376G, P377G, P377M, A378C, A378T, A378V, T379C, T379V, T379I, T379L, T379A, I380F, I380C, I380H, I380S, I380M, I380T, I380V, M381N, M381F, M381L, M381C, M381V, V382L, V382N, V382C, V382T, V382P, H383D, H383N, H383R, H383P, H383Y, E384P, E384N, E384D, E384W, P385G, P385D, I386D, I386V, I386L, I386T, G387D, G387R, G387N, G387H, G387E, R388K, R388H, S389A, S389P, S389L, V391S, V391T, I392V, I392C, V394R, V394K, V394I, V394Q, V394L, P395A, L396F, L396I, L396M, L396V, G398D, G398H, G398N, A399M, A399V, A399T, A399C, A399I, E400D, E400Q, V401M, V401I, V401C, V401L, V401A, K402R, L404V, P405D, D406E, D407P, M409Q, M409E, M409G, C411D, C411S, C411V, C411G, C411N, L413I, L413V, R414W, R414L, R414V, R414C, R414Q, V415I, K416R, G417S, P418D, N419H, N419S, N419Q, N419M, I420V, M421T, M421F, F425H, F425L, K430A, A432K, E433R, E433D, A434T, A434V, F435I, F435M, F435L, L441F, L441M, L441Y, V442K, V442H, V442Y, V442R, V442C, T443S, A446M, A446I, A446L, A446V, V447A, V447G, F449I, V450D, V450M, D451E, D452P, A453G, P455E, P455G, P455R, S456D, S456E, S456Q, S456N, G458N, G458F, G458Y, G458W, L459Y, V460Y, V460F, V460I, V460R, V460W, F461I, F461L, F461Y, D462I, D462T, D462V, G463D, R464D, V465L, S466D, S466K, S466A, E467K, D468M, F469L, K470V, L471T, L471V, T473S, T473N, G474E, W476N, W476Y, W476L, W476F, W476V, W476I, Q478T, Q478L, A479P, A479V, G480L, G480I, G480S, G480P, G480A, G480V, V481P, V481E, R483E, D485A, D485L, D485E, D485R, L490C, G492P, L493F, D496H, V497A, V498C, H502P, R504H, I507Y, I507V, I507W, G508V, F510A, I511V, I511L, F512I, P513V, L514N, P515D, P515S, G516A, V518I, S519T, T521P, T521W, V526L, V526M, I527L, S532R, S532H, I535L, R540Q, M542Y, M542W, A546N, R552Q, N554E, I558F, S560D, E561D, P563F, P563L, P563W, Q564D, Q564K, Q564S, Q564L, Q564T, Q564R, L565M, L565I, L565V, L565P, L565T, K566A, K566E, K566D, K566P, D567K, D567Q, D567T, D567N, D567S, D567A, H568G, H568N, H568D, E569M, E569K, I570V, I570L, D572R, D572P, K573T, K573S, G574M, G574L, S575A, S575Y, S575K, S575T, N577Y, N577A, N577L, I578Q, I578A, T583D, R584N, R585H, R585L, D587A, L588Q, R591Q, R591E, Y593A, N595E, E596K, P598D, A599S, I601L, and any combination thereof.

30. The engineered ACL of claim 29, wherein the engineered ACL comprises one or more amino acid alterations selected from the group consisting of: D5E, D5R, A6L, S7K, S7T, G8P, E10D, E10N, E10R, E10P, E10K, L12A, L12V, L12M, L12I, P13C, H14Q, H14P, H14A, D15S, D15E, V16I, V16C, E19D, E19R, S20H, S20K, S20R, S20D, P22E, P22D, S25N, L28M, V37C, P39R, N40T, T41V, T41I, T41M, A43S, L45I, L45V, K50R, K50L, K50I, K50V, K50F, E51R, P53L, A57F, I58F, I58M, I58L, I58V, A59V, R61P, A62D, G63D, R67K, H69F, H69L, H69Y, Y71W, Y71F, N72A, N72R, L74F, L75D, N76D, N76T, N76A, N76R, M77R, V78A, N80K, V81C, G83S, G83N, A84Y, A84H, L85F, L86Q, L86A, A87R, R88L, A92K, D93G, S94R, P95R, V97A, V98M, M99L, S100C, G101P, G103R, V104A, V104L, V104W, H106F, I108V, L109V, S110A, S110M, A113C, V116I, P119T, P119V, P1191, I120Y, I120V, I120F, I120L, I120M, I120A, V121T, L123I, L123M, L123V, A124S, A124N, A124Y, E125P, E125T, E125W, Q126R, Q126A, Q126N, Q126M, Y127L, Y127W, Y127F, S128R, I130M, P131E, R136E, R136K, R136Q, L137M, V140M, V140I, V140C, L141M, E142Q, E142N, K145R, K145D, K145G, A147S, M148V, M148L, M148A, A149V, F150V, V151C, S159E, S159R, L161I, L163V, P164A, Q165R, E167R, G168H, V169I, E170I, E170L, E170V, A173V, K175R, T176D, D177E, V181E, V183I, A185S, F186Y, A187D, L189V, L190V, G191E, G191A, H192T, G193A, Q194S, G195S, G196P, H201L, H201A, G205T, T208D, L209I, L209T, L209V, K211L, I212L, I212M, L213I, L213M, L213V, F214Y, T215S, S218T, S219T, S220G, D221K, D221L, D221T, D221H, D221R, D221M, P222S, G224A, L226M, L226V, L226I, T227I, T228S, R230H, R230K, M232L, C233A, A234S, N235Q, Q236M, T237A, T237M, T237Q, Q238M, Q238H, I239V, I239L, I239M, S240A, D241Q, D241E, D241H, D241A, A242C, A242V, A242T, L243W, L243F, L243M, L243I, L243V, R244P, R244Q, R244G, R244H, R244E, F245L, F245M, F245W, L246I, K247R, K247P, R252V, R252H, I253L, I253V, I253M, L254M, L254I, L254V, D255C, D255V, D255T, D255N, W256Y, W256V, W256F, N260S, N260H, N260F, N260Y, V262T, V262C, V262A, F263Y, G264A, G265M, S266N, S266M, H267M, H267F, H267L, H267W, H267G, N268M, N268A, N270M, M271L, M271C, M271A, M272A, M272V, M272T, M272P, M272C, L273I, L273V, L273M, L273F, A274C, A274M, A274H, A274R, A274Y, N275H, N275C, G277A, S278T, S278A, S278C, L279M, L279I, L279V, I281V, I281L, N283E, N283D, N283P, N283R, K285R, K285F, K285D, P286D, P286F, T287A, T287P, T287V, G290A, F291M, F291I, F291L, F291V, E292H, T294A, L295V, L295I, L295M, R296Q, R296D, I298M, I298V, I298L, K299A, E300Q, P302Q, G303A, G303P, G303C, G303V, T304S, L305V, L305M, L305I, A306I, A306V, A306Y, F307M, F307W, F307P, N308G, V311T, V311A, V311R, G312F, G312V, G312M, Y313F, Y313W, A314Q, A314H, A314E, M315A, M315K, M315L, M315R, L316I, L316M, N318D, N318H, E321R, E321Q, R322A, R322K, R322H, R322Q, Y330F, F331G, E333S, L334V, L334I, L334M, D335K, L336V, L336T, L336M, L336Y, L336W, L336I, L336F, I337L, I337V, I337M, F338L, F338M, F338V, F338W, Y339T, Y339S, Y339F, A340G, A342S, S343P, S343A, L344I, L344C, L344M, L344V, P345S, Q346E, Q346V, Q346A, Q346P, D347E, V348I, V348L, V348T, W349Y, W349F, W349L, S351R, S351Q, S351W, L352I, L352M, E353R, R354K, F355A, M357F, M357I, M357V, M357A, M357G, M357L, M357W, E358Q, E359Q, S362C, S362H, S362L, L363V, L363I, L363G, L365M, L365I, L365V, M366I, M366L, M366F, M366V, A367V, A367I, A367L, A367M, A367T, A367C, S368N, S369A, S369G, W370Y, T375C, T375A, A376G, A376S, P377G, A378T, T379V, T379C, T379L, T379A, T379I, 1380S, I380V, I380H, I380T, M381L, M381C, V382T, V382C, V382N, V382L, V382P, H383Y, H383R, E384N, E384W, E384D, P385G, I386V, I386T, I386L, G387R, G387H, G387N, G387D, G387E, R388K, R388H, S389A, S389P, S389L, V391S, V391T, I392V, V394I, V394K, L396V, G398H, A399C, A399I, A399T, A399V, E400Q, E400D, V401L, V401I, V401C, V401A, V401M, L404V, D406E, D407P, M409G, M409Q, M409E, C411S, R414L, R414C, K416R, N419Q, N419H, N419M, N419S, I420V, M421F, M421T, F425L, F425H, K430A, A432K, E433R, E433D, A434V, A434T, F435M, L441F, L441M, V442Y, V442R, V442K, V442C, V442H, T443S, A446L, A446V, A446M, A446I, V447A, V450M, V450D, D451E, D452P, A453G, S456N, S456D, V460F, V460I, V460R, V460W, V460Y, F461Y, F461L, D462V, D462I, G463D, V465L, S466A, S466D, S466K, E467K, D468M, F469L, K470V, L471T, L471V, T473N, T473S, W476Y, W476F, W476V, W476L, W476I, W476N, Q478T, A479V, A479P, G480S, G480A, G480P, G480L, V481P, V481E, D485L, D485E, D485R, D485A, L490C, G492P, V498C, I507V, G508V, F510A, 1511V, 1511L, F512I, L514N, P515D, P515S, G516A, V518I, S519T, T521P, T521W, V526M, V526L, I527L, S532R, S532H, I535L, R540Q, M542Y, M542W, A546N, R552Q, N554E, S560D, E561D, P563L, P563F, Q564S, Q564T, Q564L, Q564D, Q564R, Q564K, L565T, L565M, L565P, L565V, L565I, K566D, K566A, K566E, K566P, D567A, D567S, D567Q, D567N, D567T, D567K, H568G, H568D, H568N, E569M, E569K, I570V, I570L, D572P, K573S, K573T, G574M, G574L, S575T, S575Y, N577A, N577L, I578A, I578Q, T583D, R584N, R585H, D587A, L588Q, R591Q, R591E, Y593A, N595E, E596K, P598D, A599S, I601L and any combination thereof.

31. The engineered ACL of claim 29 or 30, wherein the engineered ACL comprises one or more amino acid alterations selected from the group consisting of: D5E, D5R, A6L, S7K, S7T, G8P, E10D, E10N, E10R, E10P, L12A, L12V, L12M, H14Q, H14P, D15S, D15E, V161, E19D, E19R, S20H, S20K, S20R, S20D, P22E, P22D, L28M, P39R, T41V, A43S, L45I, K50R, K50L, K50I, K50V, K50F, A57F, I58F, I58M, I58L, I58V, R61P, A62D, G63D, R67K, H69F, H69L, H69Y, Y71W, Y71F, N72A, N72R, N76D, N76T, N76A, N76R, M77R, V78A, V81C, A84Y, L85F, L86Q, A87R, A92K, D93G, S94R, P95R, V98M, V104A, 1108V, L109V, A113C, V116I, P119T, I120Y, I120V, I120F, I120L, I120M, I120A, L123I, A124S, A124N, E125P, E125T, E125W, Q126R, Q126A, Q126N, Y127L, Y127W, I130M, P131E, R136E, R136K, V140M, V140I, V140C, L141M, E142Q, E142N, K145R, A147S, M148V, A149V, V151C, S159E, S159R, L161I, L163V, P164A, E167R, G168H, V169I, E170I, E170L, E170V, A173V, K175R, T176D, D177E, V181E, V183I, A185S, F186Y, A187D, L190V, G191E, G191A, G193A, Q194S, G195S, H201L, H201A, G205T, L209I, F214Y, S218T, S219T, S220G, D221K, D221L, D221T, D221H, L226M, L226V, T228S, R230H, R230K, M232L, A234S, T237A, Q238M, I239V, D241Q, D241E, A242C, A242V, A242T, L243W, L243F, L243M, R244P, R244Q, L246I, K247R, I253L, I253V, L254M, L254I, L254V, N260S, N260H, V262T, F263Y, S266N, H267M, H267F, M272A, L273I, L273V, L273M, L273F, A274C, S278T, S278A, L279M, I281V, N283E, K285R, G290A, F291M, F291I, F291L, F291V, E292H, L295V, L295I, L295M, R296Q, I298M, I298V, E300Q, P302Q, G303A, G303P, T304S, L305V, L305M, L305I, F307M, V311T, V311A, Y313F, A314Q, A314H, M315A, M315K, M315L, M315R, L316I, N318D, N318H, E321R, E321Q, R322A, R322K, R322H, R322Q, Y330F, E333S, L334V, L334I, L336V, L336T, L336M, L336Y, I337L, I337V, F338L, F338M, F338V, Y339T, A340G, L344I, L344C, L344M, P345S, Q346E, D347E, V348I, V348L, V348T, W349Y, W349F, S351R, S351Q, L352I, E353R, R354K, F355A, M357F, M357I, M357V, M357A, M357G, M357L, M357W, E358Q, E359Q, S362C, S362H, L363V, L363I, L365M, A367V, A367I, A367L, A367M, S369A, T375C, A376G, A376S, T379V, T379C, T379L, V382T, V382C, H383Y, E384N, I386V, I386T, G387R, G387H, G387N, G387D, R388K, R388H, S389A, S389P, S389L, A399C, A399I, A399T, A399V, E400Q, V401L, V401I, V401C, L404V, D407P, M409G, K416R, N419Q, I420V, M421F, K430A, A432K, E433R, E433D, A434V, L441F, V442Y, V442R, V442K, V442C, V442H, A446L, A446V, A446M, A446I, V450M, D452P, A453G, S456N, S456D, V460F, V460I, V460R, F461Y, V465L, S466A, S466D, L471T, T473N, T473S, W476Y, W476F, W476V, W476L, W476I, A479V, A479P, G480S, G480A, G480P, V481P, V481E, D485L, D485E, D485R, D485A, L490C, G492P, I507V, 1511V, L514N, P515D, P515S, G516A, V518I, S519T, T521P, T521W, V526M, V526L, I527L, S532R, S532H, R540Q, M542Y, A546N, N554E, E561D, Q564S, Q564T, L565T, L565M, L565P, L565V, L565I, K566D, D567A, D567S, D567Q, D567N, H568G, H568D, H568N, I570V, D572P, G574M, S575T, N577A, R584N, D587A, N595E, E596K, A599S, and any combination thereof.

32. The engineered ACL of any one of claims 29 to 31, wherein the engineered ACL comprises one or more amino acid alterations selected from the group consisting of: D5E, D5R, A6L, S7K, S7T, G8P, E10D, E10N, L12A, H14Q, H14P, D15S, D15E, E19D, S20H, S20K, S20R, P22E, T41V, L45I, K50R, K50L, K50I, K50V, I58F, I58M, I58L, A62D, G63D, R67K, H69F, H69L, H69Y, N72A, N72R, N76D, N76T, N76A, N76R, M77R, V81C, L86Q, A87R, A92K, D93G, S94R, P95R, V98M, V104A, I108V, A113C, V116I, I120Y, I120V, I120F, I120L, L123I, A124S, E125P, E125T, Q126R, Q126A, Q126N, Y127L, I130M, R136E, V140M, V140I, L141M, E142Q, E142N, K145R, A147S, M148V, A149V, V151C, S159E, L161I, L163V, P164A, E167R, G168H, V169I, K175R, T176D, D177E, A185S, F186Y, A187D, L190V, G191E, G191A, G193A, G195S, H201L, H201A, G205T, L209I, F214Y, S219T, S220G, D221K, T228S, R230H, R230K, M232L, A234S, T237A, Q238M, I239V, D241Q, D241E, A242C, A242V, A242T, L243W, L243F, L243M, R244P, R244Q, L246I, K247R, I253L, L254M, N260S, N260H, F263Y, S266N, H267M, H267F, M272A, L273I, A274C, S278T, S278A, L279M, I281V, N283E, F291M, F291I, F291L, F291V, E292H, L295V, L295I, L295M, I298M, I298V, E300Q, P302Q, G303A, G303P, T304S, L305V, F307M, V311T, V311A, Y313F, A314Q, A314H, M315A, M315K, N318D, E321R, E321Q, R322A, R322K, R322H, R322Q, Y330F, E333S, L334V, L334I, L336V, L336T, L336M, I337L, I337V, F338L, F338M, A340G, L344I, V348I, V348L, W349Y, S351R, S351Q, E353R, R354K, F355A, M357F, M357I, E358Q, E359Q, S362C, S362H, L363V, L365M, A367V, A367I, A367L, A367M, S369A, A376G, T379V, T379C, V382T, V382C, H383Y, I386V, I386T, G387R, G387H, G387N, R388K, S389A, S389P, A399C, A399I, A399T, A399V, E400Q, V401L, V401I, L404V, D407P, M409G, I420V, M421F, K430A, A432K, E433R, E433D, A434V, V442Y, V442R, V442K, V442C, V442H, A446L, A446V, A446M, A446I, V450M, D452P, A453G, S456N, S456D, V460F, V460I, F461Y, V465L, S466A, T473N, T473S, W476Y, W476F, W476V, W476L, W476I, A479V, A479P, G480S, G480A, V481P, V481E, D485L, D485E, D485R, D485A, G492P, I507V, L514N, P515D, P515S, G516A, V518I, S519T, T521P, V526M, V526L, I527L, S532R, S532H, R540Q, M542Y, N554E, Q564S, Q564T, L565T, L565M, L565P, L565V, L565I, D567A, D567S, D567Q, D567N, H568G, H568D, I570V, D572P, S575T, R584N, D587A, N595E, E596K, and any combination thereof.

33. The engineered ACL of any one of claims 1 to 7 or claims 21 to 32, wherein the engineered ACL comprises a single alteration or a combination of alterations described in TABLE 9.

34. The engineered ACL of any one of claims 1 to 33, wherein the amino acid sequence, other than the one or more amino acid alterations, has at least 65%, at least 70%, at least 75%, at least 80%, at least 85%, at least 90%, at least 95%, at least 98%, at least 99%, or 100% sequence identity to the amino acid sequence referenced in SEQ ID NO: 1 or SEQ ID NO: 2.

35. A recombinant nucleic acid encoding the engineered ACL of any one of claims 1 to 34.

36. The recombinant nucleic acid of claim 35, wherein the nucleic acid comprises a nucleotide sequence encoding the engineered ACL operatively linked to a promoter.

37. A vector comprising the recombinant nucleic acid of claims 35 or 36.

38. An engineered aldehyde dehydrogenase (ALDH) comprising a variant of amino acid sequence SEQ ID NO: 3 or a functional fragment thereof, wherein the engineered ALDH comprises one or more alterations at a position described in TABLE 12.

39. The engineered ALDH of claim 38, wherein the engineered ALDH is capable of:
a) catalyzing the conversion of HHA-CoA to 6-hydroxyhexanal (6-HHAI);
b) catalyzing the conversion of NADH to NAD+;
c) catalyzing the conversion of HHA-CoA to 6-HHAI at a greater rate as compared to the wild-type ALDH;
d) having a higher affinity for a HHA-CoA substrate as compared to the wild-type ALDH;
e) catalyzing the conversion of 6ACA-CoA to 6-aminocaproate semialdehyde (6ACA-SADH);
f) catalyzing the conversion of 6ACA-CoA to 6ACA-SADH at a greater rate as compared to the wild-type ALDH;
g) having a higher affinity for a 6ACA-CoA substrate as compared to the wild-type ALDH; or
h) any combination thereof.

40. The engineered ALDH of claim 38 or 39, wherein the engineered ALDH is capable of:
a) catalyzing the conversion of adipyl-CoA to adipate semialdehyde;
b) catalyzing the conversion of NADH to NAD+;
c) catalyzing the conversion of adipyl-CoA to adipate semialdehyde at a greater rate as compared to the wild-type ALDH;
d) having a higher affinity for an adipyl-CoA substrate as compared to the wild-type ALDH;
e) or any combination thereof.

41. The engineered ALDH of any one of claims 38 to 40, wherein the engineered ALDH has higher specificity for conversion of HHA-CoA to 6-HHAI, or 6ACA-CoA to 6ACA-SADH, or both, over conversion of:
a) adipyl-CoA to adipate semialdehyde;
b) acetyl-CoA to acetaldehyde;
c) succinyl-CoA to succinate semialdehyde;
d) 3-hydroxyadipyl-CoA to 4-hydroxy-6-oxohexanoate;
e) 3HB-CoA to 3-hydroxybutyrate aldehyde (3HB-Ald); or
f) any combination thereof.

42. The engineered ALDH of any one of claims 38 to 41, wherein the engineered ALDH has higher specificity for conversion of HHA-CoA to 6-HHAI over conversion of acetyl-CoA to acetaldehyde, succinyl-CoA to succinate semialdehyde, or both.

43. The engineered ALDH of any one of claims 38 to 41, wherein the engineered ALDH has higher specificity for conversion of adipyl-CoA to adipate semialdehyde over conversion of acetyl-CoA to acetaldehyde, succinyl-CoA to succinate semialdehyde, or both.

44. The engineered ALDH of any one of claims 38 to 41, wherein the engineered ALDH has a lower or equal affinity for succinyl-CoA as compared to the wild-type ALDH.

45. The engineered ALDH of any one of claims 38 to 44, wherein the engineered ALDH comprises an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an ALDH consisting of the amino acid sequence of SEQ ID NO: 3.

46. The engineered ALDH of any one of claims 38 to 45, wherein the one or more amino acid alterations comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 or more alterations.

47. The engineered ALDH of any one of claims 38 to 46, wherein the one or more amino acid alterations are conservative or non-conservative amino acid substitutions, deletions, or insertions, or any combination thereof.

48. The engineered ALDH of any one of claims 38 to 47, wherein the ALDH comprises one or more amino acid alterations at a position corresponding to position 2, 3, 4, 5, 6, 7, 8,9, 10, 11, 12, 13, 14, 15, 16, 17, 18, 20, 21, 22, 23, 24, 25, 26, 27, 28, 29, 30, 31, 32, 33, 34, 35, 36, 37, 38, 39, 40, 41, 42, 43, 44, 45, 47, 48, 49, 50, 51, 52, 53, 54, 55, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 104, 106, 107, 108, 109, 110, 111, 112, 113, 114, 115, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, 201, 202, 203, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 243, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 288, 289, 290, 291, 292, 293, 294, 295, 296, 297, 298, 299, 300, 301, 302, 303, 304, 305, 306, 307, 308, 310, 311, 312, 313, 314, 315, 316, 317, 318, 319, 320, 321, 322, 323, 324, 325, 326, 327, 328, 329, 330, 331, 332, 333, 334, 335, 337, 338, 339, 341, 342, 343, 344, 345, 346, 347, 348, 349, 350, 351, 352, 354, 355, 356, 357, 358, 359, 360, 361, 362, 363, 364, 366, 368, 369, 370, 371, 372, 373, 374, 375, 376, 377, 378, 379, 380, 381, 382, 383, 384, 385, 386, 387, 388, 389, 390, 391, 392, 393, 394, 396, 397, 398, 399, 400, 401, 402, 403, 405, 406, 407, 408, 410, 411, 412, 413, 414, 415, 416, 418, 420, 421, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, or any combination thereof, in SEQ ID NO: 3.

49. The engineered ALDH of any one of claims 38 to 48, wherein the ALDH comprises one or more amino acid alterations at a position corresponding to position 2, 3, 4, 5, 6, 7, 9, 11, 12, 15, 16, 17, 18, 20, 21, 22, 23, 24, 25, 26, 27, 28, 30, 31, 32, 33, 34, 35, 36, 37, 38, 39, 40, 41, 42, 43, 44, 45, 47, 48, 49, 51, 52, 54, 55, 58, 61, 62, 63, 64, 65, 66, 67, 70, 71, 73, 74, 75, 77, 78, 79, 80, 81, 82, 83, 84, 86, 88, 89, 90, 91, 92, 93, 94, 95, 96, 97, 98, 99, 100, 101, 102, 104, 107, 111, 117, 119, 120, 121, 122, 124, 125, 126, 128, 129, 130, 133, 136, 144, 145, 146, 147, 148, 149, 150, 151, 152, 153, 154, 155, 156, 157, 158, 159, 160, 162, 164, 165, 166, 167, 168, 169, 170, 171, 172, 174, 175, 176, 177, 180, 181, 183, 184, 187, 189, 190, 194, 196, 197, 198, 199, 200, 201, 202, 203, 206, 207, 208, 212, 214, 215, 216, 217, 218, 219, 221, 222, 223, 224, 225, 227, 228, 229, 230, 231, 232, 233, 234, 237, 238, 240, 243, 245, 246, 248, 249, 250, 251, 252, 253, 254, 255, 256, 257, 258, 259, 260, 261, 262, 263, 264, 265, 266, 267, 270, 271, 272, 273, 274, 275, 276, 277, 278, 279, 280, 281, 282, 283, 284, 285, 286, 288, 290, 291, 293, 294, 295, 296, 299, 300, 301, 302, 303, 304, 305, 306, 307, 308, 310, 311, 312, 313, 315, 316, 317, 318, 319, 320, 321, 322, 323, 324, 325, 326, 327, 328, 329, 330, 331, 332, 333, 334, 335, 339, 341, 342, 343, 344, 345, 346, 347, 348, 349, 350, 351, 354, 355, 356, 358, 359, 360, 362, 363, 366, 370, 371, 372, 373, 374, 375, 376, 377, 378, 379, 380, 381, 382, 383, 384, 385, 386, 387, 388, 389, 390, 392, 393, 394, 396, 397, 400, 401, 402, 405, 406, 407, 410, 411, 414, 420, 421, 423, 425, 426, 427, 428, 431, 433, 435, 436, 437, 438, 439, 440, 441, 443, 444, 446, 447, 448, 449, 450, 452, 453, 454, 455, 457, 458, 459 or a combination thereof, in SEQ ID NO: 3.

50. The engineered ALDH of any one of claims 38 to 49, wherein the ALDH comprises one or more amino acid alterations at a position corresponding to position 42, 119, 149, 158, 174, 246, 259, 263, 299, 327, 348, 370, 392, 401, 427 or a combination thereof, in SEQ ID NO: 3.

51. The engineered ALDH of any one of claims 38 to 48, wherein the one or more amino acid alterations result in an engineered ALDH comprising:
a) P, Q, T at a residue corresponding to position 2 in SEQ ID NO: 3;
b) E, K, M, R, S at a residue corresponding to position 3 in SEQ ID NO: 3;
c) G, S at a residue corresponding to position 4 in SEQ ID NO: 3;
d) I, T at a residue corresponding to position 5 in SEQ ID NO: 3;
e) E, F, H, P, Q, R at a residue corresponding to position 6 in SEQ ID NO: 3;
f) E, Q at a residue corresponding to position 7 in SEQ ID NO: 3;
g) D at a residue corresponding to position 8 in SEQ ID NO: 3;
h) I, V at a residue corresponding to position 9 in SEQ ID NO: 3;
i) N at a residue corresponding to position 10 in SEQ ID NO: 3;
j) A, D, E, Q, R at a residue corresponding to position 11 in SEQ ID NO: 3;
k) A, I, L at a residue corresponding to position 12 in SEQ ID NO: 3;
l) L at a residue corresponding to position 13 in SEQ ID NO: 3;
m) D, E, K, Q at a residue corresponding to position 14 in SEQ ID NO: 3;
n) K, R at a residue corresponding to position 15 in SEQ ID NO: 3;
o) A at a residue corresponding to position 16 in SEQ ID NO: 3;
p) H, K, Q at a residue corresponding to position 17 in SEQ ID NO: 3;
q) E, K, Q, T at a residue corresponding to position 18 in SEQ ID NO: 3;
r) F, H, M at a residue corresponding to position 20 in SEQ ID NO: 3;
s) A, E, K, P, R at a residue corresponding to position 21 in SEQ ID NO: 3;
t) A, K, Q at a residue corresponding to position 22 in SEQ ID NO: 3;
u) L, W, Y at a residue corresponding to position 23 in SEQ ID NO: 3;
v) E, K, R, S at a residue corresponding to position 24 in SEQ ID NO: 3;
w) A, E, K, N, S at a residue corresponding to position 25 in SEQ ID NO: 3;
x) F, L, M, T, W at a residue corresponding to position 26 in SEQ ID NO: 3;
y) D, S, T at a residue corresponding to position 27 in SEQ ID NO: 3;
z) W at a residue corresponding to position 28 in SEQ ID NO: 3;
aa) K, Q at a residue corresponding to position 29 in SEQ ID NO: 3;
bb) D, E, K, Q, R at a residue corresponding to position 30 in SEQ ID NO: 3;
cc) I, L, R at a residue corresponding to position 31 in SEQ ID NO: 3;
dd) A, E, N at a residue corresponding to position 32 in SEQ ID NO: 3;
ee) A, E, Q, R at a residue corresponding to position 33 in SEQ ID NO: 3;
ff) I, L, M, V at a residue corresponding to position 34 in SEQ ID NO: 3;
gg) C, I, L, M at a residue corresponding to position 35 in SEQ ID NO: 3;
hh) E, H, K at a residue corresponding to position 36 in SEQ ID NO: 3;
ii) A, K, R at a residue corresponding to position 37 in SEQ ID NO: 3;
jj) F, I, L, M, W at a residue corresponding to position 38 in SEQ ID NO: 3;
kk) A, R at a residue corresponding to position 39 in SEQ ID NO: 3;
ll) D, E, Q, R, W at a residue corresponding to position 40 in SEQ ID NO: 3;
mm) L, V at a residue corresponding to position 41 in SEQ ID NO: 3;
nn) C, L, M, V at a residue corresponding to position 42 in SEQ ID NO: 3;
oo) E, L, R at a residue corresponding to position 43 in SEQ ID NO: 3;
pp) E, K, Q, R at a residue corresponding to position 44 in SEQ ID NO: 3;
qq) H, Q, R at a residue corresponding to position 45 in SEQ ID NO: 3;
rr) D at a residue corresponding to position 47 in SEQ ID NO: 3;
ss) D, E, M, R at a residue corresponding to position 48 in SEQ ID NO: 3;
tt) F, I, M, W, Y at a residue corresponding to position 49 in SEQ ID NO: 3;
uu) G at a residue corresponding to position 50 in SEQ ID NO: 3;
vv) A, E, K, N, Q, R at a residue corresponding to position 51 in SEQ ID NO: 3;
ww) L at a residue corresponding to position 52 in SEQ ID NO: 3;
xx) E, I, S at a residue corresponding to position 53 in SEQ ID NO: 3;
yy) I, L, M, T at a residue corresponding to position 54 in SEQ ID NO: 3;
zz) E, Q, R at a residue corresponding to position 55 in SEQ ID NO: 3;
aaa) E, G, Q, R at a residue corresponding to position 58 in SEQ ID NO: 3;
bbb) F, I, L, Y at a residue corresponding to position 59 in SEQ ID NO: 3;
ccc) A at a residue corresponding to position 60 in SEQ ID NO: 3;
ddd) D, E, K, R at a residue corresponding to position 61 in SEQ ID NO: 3;
eee) A, F, I, M, V, W at a residue corresponding to position 62 in SEQ ID NO: 3;
fff) D, E, Q at a residue corresponding to position 63 in SEQ ID NO: 3;
ggg) E, H at a residue corresponding to position 64 in SEQ ID NO: 3;
hhh) E, R at a residue corresponding to position 65 in SEQ ID NO: 3;
iii) L, V at a residue corresponding to position 66 in SEQ ID NO: 3;
jjj) E at a residue corresponding to position 67 in SEQ ID NO: 3;
kkk) D, E at a residue corresponding to position 68 in SEQ ID NO: 3;
lll) H, I, L, M, N at a residue corresponding to position 69 in SEQ ID NO: 3;
mmm) I, L, M, R at a residue corresponding to position 70 in SEQ ID NO: 3;
nnn) D, E, F, H, Y at a residue corresponding to position 71 in SEQ ID NO: 3;
ooo) A, R at a residue corresponding to position 72 in SEQ ID NO: 3;
ppp) A, T at a residue corresponding to position 73 in SEQ ID NO: 3;
qqq) D, E, R at a residue corresponding to position 74 in SEQ ID NO: 3;
rrr) G, P, Y at a residue corresponding to position 75 in SEQ ID NO: 3;
sss) F, I, L, M, Y at a residue corresponding to position 76 in SEQ ID NO: 3;
ttt) F, Y at a residue corresponding to position 77 in SEQ ID NO: 3;
uuu) E, F, H, N, R, Y at a residue corresponding to position 78 in SEQ ID NO: 3;
vvv) D, S, W, Y at a residue corresponding to position 79 in SEQ ID NO: 3;
www) F, I, L, V at a residue corresponding to position 80 in SEQ ID NO: 3;
xxx) A, E, R at a residue corresponding to position 81 in SEQ ID NO: 3;
yyy) E, G, P at a residue corresponding to position 82 in SEQ ID NO: 3;
zzz) E, Q at a residue corresponding to position 83 in SEQ ID NO: 3;
aaaa) Q, R at a residue corresponding to position 84 in SEQ ID NO: 3;
bbbb) A, T at a residue corresponding to position 85 in SEQ ID NO: 3;
cccc) E, K, V at a residue corresponding to position 86 in SEQ ID NO: 3;
dddd) T at a residue corresponding to position 87 in SEQ ID NO: 3;
eeee) L, M, V at a residue corresponding to position 88 in SEQ ID NO: 3;
ffff) L, P, V at a residue corresponding to position 89 in SEQ ID NO: 3;
gggg) D, E, H, P, R, S at a residue corresponding to position 90 in SEQ ID NO: 3;
hhhh) D, E, Q at a residue corresponding to position 91 in SEQ ID NO: 3;
iiii) D, M at a residue corresponding to position 92 in SEQ ID NO: 3;
jjjj) D, E at a residue corresponding to position 93 in SEQ ID NO: 3;
kkkk) A, D, G, N at a residue corresponding to position 94 in SEQ ID NO: 3;
llll) K, M, N, R at a residue corresponding to position 95 in SEQ ID NO: 3;
mmmm) H, K, N, R at a residue corresponding to position 96 in SEQ ID NO: 3;
nnnn) I, M, V at a residue corresponding to position 97 in SEQ ID NO: 3;
oooo) I, M, T, Y at a residue corresponding to position 98 in SEQ ID NO: 3;
pppp) K, Q, R, T at a residue corresponding to position 99 in SEQ ID NO: 3;
qqqq) Y, L, M, I at a residue corresponding to position 100 in SEQ ID NO: 3;
rrrr) K, Y, R, H at a residue corresponding to position 101 in SEQ ID NO: 3;
ssss) E, Q, R, V at a residue corresponding to position 102 in SEQ ID NO: 3;
tttt) V, Y, F, L, M at a residue corresponding to position 104 in SEQ ID NO: 3;
uuuu) I at a residue corresponding to position 106 in SEQ ID NO: 3;
vvvv) C, V at a residue corresponding to position 107 in SEQ ID NO: 3;
wwww) A, C at a residue corresponding to position 108 in SEQ ID NO: 3;
xxxx) I, A, M at a residue corresponding to position 109 in SEQ ID NO: 3;
yyyy) M, L, I at a residue corresponding to position 110 in SEQ ID NO: 3;
zzzz) T, V, Y, I, S at a residue corresponding to position 111 in SEQ ID NO: 3;
aaaaa) E at a residue corresponding to position 112 in SEQ ID NO: 3;
bbbbb) F, S, W, Y at a residue corresponding to position 113 in SEQ ID NO: 3;
ccccc) N at a residue corresponding to position 114 in SEQ ID NO: 3;
ddddd) W, R, H, Y at a residue corresponding to position 115 in SEQ ID NO: 3;
eeeee) N, L, T, I at a residue corresponding to position 117 in SEQ ID NO: 3;
fffff) N, A, L, M, S, F, V at a residue corresponding to position 118 in SEQ ID NO: 3;
ggggg) Q, I, L, M, T, S at a residue corresponding to position 119 in SEQ ID NO: 3;
hhhhh) T, A, P, M at a residue corresponding to position 120 in SEQ ID NO: 3;
iiiii) V, A, L, C, I at a residue corresponding to position 121 in SEQ ID NO: 3;
jjjjj) C, F, R, Y, H, N, S, G, V, W at a residue corresponding to position 122 in SEQ ID NO: 3;
kkkkk) K, P at a residue corresponding to position 123 in SEQ ID NO: 3;
lllll) V, L, M, I at a residue corresponding to position 124 in SEQ ID NO: 3;
mmmmm) I, L, A, V at a residue corresponding to position 125 in SEQ ID NO: 3;
nnnnn) A, P at a residue corresponding to position 126 in SEQ ID NO: 3;
ooooo) M at a residue corresponding to position 127 in SEQ ID NO: 3;
ppppp) I at a residue corresponding to position 128 in SEQ ID NO: 3;
qqqqq) A, M at a residue corresponding to position 129 in SEQ ID NO: 3;
rrrrr) T, S, A at a residue corresponding to position 130 in SEQ ID NO: 3;
sssss) R at a residue corresponding to position 131 in SEQ ID NO: 3;
ttttt) C at a residue corresponding to position 132 in SEQ ID NO: 3;
uuuuu) T, C at a residue corresponding to position 133 in SEQ ID NO: 3;
vvvvv) I, C, V at a residue corresponding to position 134 in SEQ ID NO: 3;
wwwww) V, M at a residue corresponding to position 135 in SEQ ID NO: 3;
xxxxx) L, M, F, I at a residue corresponding to position 136 in SEQ ID NO: 3;
yyyyy) R, K at a residue corresponding to position 137 in SEQ ID NO: 3;
zzzzz) G at a residue corresponding to position 138 in SEQ ID NO: 3;
aaaaaa) S, G at a residue corresponding to position 139 in SEQ ID NO: 3;
bbbbbb) E at a residue corresponding to position 140 in SEQ ID NO: 3;
cccccc) Q, K, Y, E at a residue corresponding to position 141 in SEQ ID NO: 3;
dddddd) T, S at a residue corresponding to position 142 in SEQ ID NO: 3;
eeeeee) P at a residue corresponding to position 143 in SEQ ID NO: 3;
ffffff) L, N, R at a residue corresponding to position 144 in SEQ ID NO: 3;
gggggg) T, C, V at a residue corresponding to position 145 in SEQ ID NO: 3;
hhhhhh) T, C, A, N at a residue corresponding to position 146 in SEQ ID NO: 3;
iiiiii) I, M, A at a residue corresponding to position 147 in SEQ ID NO: 3;
jjjjjj) Y, L, A, E, H, R at a residue corresponding to position 148 in SEQ ID NO: 3;
kkkkkk) V, I, L, M, F at a residue corresponding to position 149 in SEQ ID NO: 3;
llllll) I, M, G, A at a residue corresponding to position 150 in SEQ ID NO: 3;
mmmmmm) D, K, R, Q at a residue corresponding to position 151 in SEQ ID NO: 3;
nnnnnn) L, M, I at a residue corresponding to position 152 in SEQ ID NO: 3;
oooooo) F, V, C, M at a residue corresponding to position 153 in SEQ ID NO: 3;
pppppp) H, A, Q, K, R at a residue corresponding to position 154 in SEQ ID NO: 3;
qqqqqq) R, K, D, Q at a residue corresponding to position 155 in SEQ ID NO: 3;
rrrrrr) I at a residue corresponding to position 156 in SEQ ID NO: 3;
ssssss) A, I, F at a residue corresponding to position 157 in SEQ ID NO: 3;
tttttt) E, V, K at a residue corresponding to position 158 in SEQ ID NO: 3;
uuuuuu) K, Q, D, E at a residue corresponding to position 159 in SEQ ID NO: 3;
vvvvvv) V, C, A at a residue corresponding to position 160 in SEQ ID NO: 3;
wwwwww) I, A, F, Y, L, V at a residue corresponding to position 162 in SEQ ID NO: 3;
xxxxxx) D, A, P at a residue corresponding to position 164 in SEQ ID NO: 3;
yyyyyy) H, G, D at a residue corresponding to position 165 in SEQ ID NO: 3;
zzzzzz) C, A, I, V at a residue corresponding to position 166 in SEQ ID NO: 3;
aaaaaaa) V, F, L at a residue corresponding to position 167 in SEQ ID NO: 3;
bbbbbbb) T, N at a residue corresponding to position 168 in SEQ ID NO: 3;
ccccccc) L, W, C, M, I at a residue corresponding to position 169 in SEQ ID NO: 3;
ddddddd) L, V at a residue corresponding to position 170 in SEQ ID NO: 3;
eeeeeee) E, P, N, D at a residue corresponding to position 171 in SEQ ID NO: 3;
fffffff) D, G, H at a residue corresponding to position 172 in SEQ ID NO: 3;
ggggggg) G, D at a residue corresponding to position 173 in SEQ ID NO: 3;
hhhhhhh) G, D, T at a residue corresponding to position 174 in SEQ ID NO: 3;
iiiiiii) I, H, R at a residue corresponding to position 175 in SEQ ID NO: 3;
jjjjjjj) Q, D at a residue corresponding to position 176 in SEQ ID NO: 3;
kkkkkkk) V, T, A, I at a residue corresponding to position 177 in SEQ ID NO: 3;
lllllll) V, A, T, G at a residue corresponding to position 178 in SEQ ID NO: 3;
mmmmmmm) D, N, E at a residue corresponding to position 179 in SEQ ID NO: 3;
nnnnnnn) H, Q, A, E at a residue corresponding to position 180 in SEQ ID NO: 3;
ooooooo) I, F at a residue corresponding to position 181 in SEQ ID NO: 3;
ppppppp) L, H, I, V at a residue corresponding to position 182 in SEQ ID NO: 3;
qqqqqqq) Q, K, D, A, H, E at a residue corresponding to position 183 in SEQ ID NO: 3;
rrrrrrr) H, P, S, G at a residue corresponding to position 184 in SEQ ID NO: 3;
sssssss) C, I at a residue corresponding to position 185 in SEQ ID NO: 3;
ttttttt) R at a residue corresponding to position 186 in SEQ ID NO: 3;
uuuuuuu) H, K, F, I, V, M at a residue corresponding to position 187 in SEQ ID NO: 3;
vvvvvvv) I, L, V at a residue corresponding to position 188 in SEQ ID NO: 3;
wwwwwww) S, F, T, M, V, L at a residue corresponding to position 189 in SEQ ID NO: 3;
xxxxxxx) P, A, F at a residue corresponding to position 190 in SEQ ID NO: 3;
yyyyyyy) R at a residue corresponding to position 191 in SEQ ID NO: 3;
zzzzzzz) S at a residue corresponding to position 193 in SEQ ID NO: 3;
aaaaaaaa) T, R, K, A at a residue corresponding to position 194 in SEQ ID NO: 3;
bbbbbbbb) A at a residue corresponding to position 195 in SEQ ID NO: 3;
cccccccc) M, I, V at a residue corresponding to position 196 in SEQ ID NO: 3;
dddddddd) I, M, G, A at a residue corresponding to position 197 in SEQ ID NO: 3;
eeeeeeee) Q, E, R at a residue corresponding to position 198 in SEQ ID NO: 3;
ffffffff) S, V at a residue corresponding to position 199 in SEQ ID NO: 3;
gggggggg) C, I, V at a residue corresponding to position 200 in SEQ ID NO: 3;
hhhhhhhh) M, Q, A at a residue corresponding to position 201 in SEQ ID NO: 3;
iiiiiiii) N, K, A, R, T, E, Q at a residue corresponding to position 202 in SEQ ID NO: 3;
jjjjjjjj) N, H, A at a residue corresponding to position 203 in SEQ ID NO: 3;
kkkkkkkk) R, T at a residue corresponding to position 205 in SEQ ID NO: 3;
llllllll) V, R, K at a residue corresponding to position 206 in SEQ ID NO: 3;
mmmmmmmm) V, S, C, T at a residue corresponding to position 207 in SEQ ID NO: 3;
nnnnnnnn) V, T, Y, M, L at a residue corresponding to position 208 in SEQ ID NO: 3;
oooooooo) L, E, M at a residue corresponding to position 209 in SEQ ID NO: 3;
pppppppp) H, L at a residue corresponding to position 210 in SEQ ID NO: 3;
qqqqqqqq) L at a residue corresponding to position 211 in SEQ ID NO: 3;
rrrrrrrr) A, P, G at a residue corresponding to position 212 in SEQ ID NO: 3;
ssssssss) K at a residue corresponding to position 213 in SEQ ID NO: 3;
tttttttt) S, K at a residue corresponding to position 214 in SEQ ID NO: 3;
uuuuuuuu) S, N, A, C, D, P at a residue corresponding to position 215 in SEQ ID NO: 3;
vvvvvvvv) H, A, P at a residue corresponding to position 216 in SEQ ID NO: 3;
wwwwwwww) F, A, I, C, M at a residue corresponding to position 217 in SEQ ID NO: 3;
xxxxxxxx) I, V, F, Y at a residue corresponding to position 218 in SEQ ID NO: 3;
yyyyyyyy) M, F, L, I at a residue corresponding to position 219 in SEQ ID NO: 3;
zzzzzzzz) C, F at a residue corresponding to position 220 in SEQ ID NO: 3;
aaaaaaaaa) D, P at a residue corresponding to position 221 in SEQ ID NO: 3;
bbbbbbbbb) T, S at a residue corresponding to position 222 in SEQ ID NO: 3;
ccccccccc) C at a residue corresponding to position 223 in SEQ ID NO: 3;
ddddddddd) N at a residue corresponding to position 224 in SEQ ID NO: 3;
eeeeeeeee) V at a residue corresponding to position 225 in SEQ ID NO: 3;
fffffffff) E at a residue corresponding to position 226 in SEQ ID NO: 3;
ggggggggg) K, R, M, E at a residue corresponding to position 227 in SEQ ID NO: 3;
hhhhhhhhh) A at a residue corresponding to position 228 in SEQ ID NO: 3;
iiiiiiiii) W, V at a residue corresponding to position 229 in SEQ ID NO: 3;
jjjjjjjjj) R, D, P, Q, K, E at a residue corresponding to position 230 in SEQ ID NO: 3;
kkkkkkkkk) Q, R, I, K at a residue corresponding to position 231 in SEQ ID NO: 3;
lllllllll) I, F, T, C, Q, M, N at a residue corresponding to position 232 in SEQ ID NO: 3;
mmmmmmmmm) V, L, M at a residue corresponding to position 233 in SEQ ID NO: 3;
nnnnnnnnn) W, Q, F, A, M at a residue corresponding to position 234 in SEQ ID NO: 3;
ooooooooo) A, S at a residue corresponding to position 235 in SEQ ID NO: 3;
ppppppppp) K, C, W, A, I at a residue corresponding to position 236 in SEQ ID NO: 3;
qqqqqqqqq) F, Y, C, M at a residue corresponding to position 237 in SEQ ID NO: 3;
rrrrrrrrr) R, M, Q, Y, W at a residue corresponding to position 238 in SEQ ID NO: 3;
sssssssss) R, H, Q at a residue corresponding to position 239 in SEQ ID NO: 3;
ttttttttt) Q, A, T, C, S at a residue corresponding to position 240 in SEQ ID NO: 3;
uuuuuuuuu) N at a residue corresponding to position 241 in SEQ ID NO: 3;
vvvvvvvvv) V, M, T, C at a residue corresponding to position 243 in SEQ ID NO: 3;
wwwwwwwww) M, I, N, S, V, A, C at a residue corresponding to position 245 in SEQ ID NO: 3;
xxxxxxxxx) S, A at a residue corresponding to position 246 in SEQ ID NO: 3;
yyyyyyyyy) G, P, I, A at a residue corresponding to position 247 in SEQ ID NO: 3;
zzzzzzzzz) D, T, E, K, S, N at a residue corresponding to position 248 in SEQ ID NO: 3;
aaaaaaaaaa) Y, R at a residue corresponding to position 249 in SEQ ID NO: 3;
bbbbbbbbbb) I, L, M at a residue corresponding to position 250 in SEQ ID NO: 3;
cccccccccc) Y, F, L, V at a residue corresponding to position 251 in SEQ ID NO: 3;
dddddddddd) V, I at a residue corresponding to position 252 in SEQ ID NO: 3;
eeeeeeeeee) D, Q, H, V, E at a residue corresponding to position 253 in SEQ ID NO: 3;
ffffffffff) Q, K, R, D at a residue corresponding to position 254 in SEQ ID NO: 3;
gggggggggg) E at a residue corresponding to position 255 in SEQ ID NO: 3;
hhhhhhhhhh) V, M, I, R at a residue corresponding to position 256 in SEQ ID NO: 3;
iiiiiiiiii) A, F, H, Y at a residue corresponding to position 257 in SEQ ID NO: 3;
jjjjjjjjjj) D at a residue corresponding to position 258 in SEQ ID NO: 3;
kkkkkkkkkk) A, R, K, D, Q at a residue corresponding to position 259 in SEQ ID NO: 3;
llllllllll) I, V, F, L at a residue corresponding to position 260 in SEQ ID NO: 3;
mmmmmmmmmm) L, M, V at a residue corresponding to position 261 in SEQ ID NO: 3;
nnnnnnnnnn) Q, D, E, K at a residue corresponding to position 262 in SEQ ID NO: 3;
oooooooooo) E, K, R at a residue corresponding to position 263 in SEQ ID NO: 3;
pppppppppp) M, V, F at a residue corresponding to position 264 in SEQ ID NO: 3;
qqqqqqqqqq) Q, R, A, E at a residue corresponding to position 265 in SEQ ID NO: 3;
rrrrrrrrrr) A, E at a residue corresponding to position 266 in SEQ ID NO: 3;
ssssssssss) Y, R, N, A, Q at a residue corresponding to position 267 in SEQ ID NO: 3;
tttttttttt) A at a residue corresponding to position 268 in SEQ ID NO: 3;
uuuuuuuuuu) S, G, C at a residue corresponding to position 269 in SEQ ID NO: 3;
vvvvvvvvvv) L, F, I, M, W at a residue corresponding to position 270 in SEQ ID NO: 3;
wwwwwwwwww) F, K, V, W, R, H at a residue corresponding to position 271 in SEQ ID NO: 3;
xxxxxxxxxx) V, M, L at a residue corresponding to position 272 in SEQ ID NO: 3;
yyyyyyyyyy) C at a residue corresponding to position 273 in SEQ ID NO: 3;
zzzzzzzzzz) E, D at a residue corresponding to position 274 in SEQ ID NO: 3;
aaaaaaaaaaa) P, G, A, E at a residue corresponding to position 275 in SEQ ID NO: 3;
bbbbbbbbbbb) Q, M, R, D at a residue corresponding to position 276 in SEQ ID NO: 3;
ccccccccccc) Q, E at a residue corresponding to position 277 in SEQ ID NO: 3;
ddddddddddd) P, E at a residue corresponding to position 278 in SEQ ID NO: 3;
eeeeeeeeeee) E, D, S at a residue corresponding to position 279 in SEQ ID NO: 3;
fffffffffff) Q, R, T at a residue corresponding to position 280 in SEQ ID NO: 3;
ggggggggggg) V, M, I at a residue corresponding to position 281 in SEQ ID NO: 3;
hhhhhhhhhhh) Q, K, M, G, E at a residue corresponding to position 282 in SEQ ID NO: 3;
iiiiiiiiiii) E, H, N at a residue corresponding to position 283 in SEQ ID NO: 3;
jjjjjjjjjjj) V, P at a residue corresponding to position 284 in SEQ ID NO: 3;
kkkkkkkkkkk) L, V, I, M at a residue corresponding to position 285 in SEQ ID NO: 3;
lllllllllll) L, I, W, Y, V at a residue corresponding to position 286 in SEQ ID NO: 3;
mmmmmmmmmmm) R, D, E at a residue corresponding to position 288 in SEQ ID NO: 3;
nnnnnnnnnnn) M at a residue corresponding to position 289 in SEQ ID NO: 3;
ooooooooooo) H at a residue corresponding to position 290 in SEQ ID NO: 3;
ppppppppppp) K, L, E, T, H, A, N at a residue corresponding to position 291 in SEQ ID NO: 3;
qqqqqqqqqqq) I, M at a residue corresponding to position 292 in SEQ ID NO: 3;
rrrrrrrrrrr) A, V, I, N at a residue corresponding to position 293 in SEQ ID NO: 3;
sssssssssss) S, N, P, E, R at a residue corresponding to position 294 in SEQ ID NO: 3;
ttttttttttt) R, K, A, Q, D at a residue corresponding to position 295 in SEQ ID NO: 3;
uuuuuuuuuuu) W, R, V, F, H, L at a residue corresponding to position 296 in SEQ ID NO: 3;
vvvvvvvvvvv) A, T, Q, V, L, H, I at a residue corresponding to position 297 in SEQ ID NO: 3;
wwwwwwwwwww) A at a residue corresponding to position 298 in SEQ ID NO: 3;
xxxxxxxxxxx) K, Q, R at a residue corresponding to position 299 in SEQ ID NO: 3;
yyyyyyyyyyy) A, R at a residue corresponding to position 300 in SEQ ID NO: 3;
zzzzzzzzzzz) I, A at a residue corresponding to position 301 in SEQ ID NO: 3;
aaaaaaaaaaaa) A, R, D, Q, E at a residue corresponding to position 302 in SEQ ID NO: 3;
bbbbbbbbbbbb) Q, W, R, E at a residue corresponding to position 303 in SEQ ID NO: 3;
cccccccccccc) L, G, Y at a residue corresponding to position 304 in SEQ ID NO: 3;
dddddddddddd) I, V at a residue corresponding to position 305 in SEQ ID NO: 3;
eeeeeeeeeeee) H, K, E, R at a residue corresponding to position 306 in SEQ ID NO: 3;
ffffffffffff) I, V, M at a residue corresponding to position 307 in SEQ ID NO: 3;
gggggggggggg) A, T at a residue corresponding to position 308 in SEQ ID NO: 3;
hhhhhhhhhhhh) G, V, L, F at a residue corresponding to position 310 in SEQ ID NO: 3;
iiiiiiiiiiii) R, T, K, S, D, E at a residue corresponding to position 311 in SEQ ID NO: 3;
jjjjjjjjjjjj) A, D, T, K at a residue corresponding to position 312 in SEQ ID NO: 3;
kkkkkkkkkkkk) L, V at a residue corresponding to position 313 in SEQ ID NO: 3;
llllllllllll) A at a residue corresponding to position 314 in SEQ ID NO: 3;
mmmmmmmmmmmm) D at a residue corresponding to position 315 in SEQ ID NO: 3;
nnnnnnnnnnnn) E, G, N at a residue corresponding to position 316 in SEQ ID NO: 3;
oooooooooooo) G, T at a residue corresponding to position 317 in SEQ ID NO: 3;
pppppppppppp) R, T, Y at a residue corresponding to position 318 in SEQ ID NO: 3;
qqqqqqqqqqqq) F, L, M, I, Y at a residue corresponding to position 319 in SEQ ID NO: 3;
rrrrrrrrrrrr) Y, L at a residue corresponding to position 320 in SEQ ID NO: 3;
ssssssssssss) L, P, C, I at a residue corresponding to position 321 in SEQ ID NO: 3;
tttttttttttt) A, T, G, V at a residue corresponding to position 322 in SEQ ID NO: 3;
uuuuuuuuuuuu) E, L, I, G, P, V, K at a residue corresponding to position 323 in SEQ ID NO: 3;
vvvvvvvvvvvv) I, V, L at a residue corresponding to position 324 in SEQ ID NO: 3;
wwwwwwwwwwww) K, E, D, T at a residue corresponding to position 325 in SEQ ID NO: 3;
xxxxxxxxxxxx) D at a residue corresponding to position 326 in SEQ ID NO: 3;
yyyyyyyyyyyy) W, F, V at a residue corresponding to position 327 in SEQ ID NO: 3;
zzzzzzzzzzzz) D, T, S at a residue corresponding to position 328 in SEQ ID NO: 3;
aaaaaaaaaaaaa) E, R, P, D, N at a residue corresponding to position 329 in SEQ ID NO: 3;
bbbbbbbbbbbbb) E at a residue corresponding to position 330 in SEQ ID NO: 3;
ccccccccccccc) S, D, H, M, Y at a residue corresponding to position 331 in SEQ ID NO: 3;
ddddddddddddd) R, T at a residue corresponding to position 332 in SEQ ID NO: 3;
eeeeeeeeeeeee) M, V, F, I at a residue corresponding to position 333 in SEQ ID NO: 3;
fffffffffffff) V, S, M, C at a residue corresponding to position 334 in SEQ ID NO: 3;
ggggggggggggg) R, Q, H at a residue corresponding to position 335 in SEQ ID NO: 3;
hhhhhhhhhhhhh) Q, E at a residue corresponding to position 337 in SEQ ID NO: 3;
iiiiiiiiiiiii) F, V, I, M at a residue corresponding to position 338 in SEQ ID NO: 3;
jjjjjjjjjjjjj) C, M at a residue corresponding to position 339 in SEQ ID NO: 3;
kkkkkkkkkkkkk) I at a residue corresponding to position 341 in SEQ ID NO: 3;
lllllllllllll) M, V, I at a residue corresponding to position 342 in SEQ ID NO: 3;
mmmmmmmmmmmmm) C, T, V, S, P at a residue corresponding to position 343 in SEQ ID NO: 3;
nnnnnnnnnnnnn) I, V, L, M at a residue corresponding to position 344 in SEQ ID NO: 3;
ooooooooooooo) Y, V, M, L, I at a residue corresponding to position 345 in SEQ ID NO: 3;
ppppppppppppp) R, K, P at a residue corresponding to position 346 in SEQ ID NO: 3;
qqqqqqqqqqqqq) F, V at a residue corresponding to position 347 in SEQ ID NO: 3;
rrrrrrrrrrrrr) E, K, S at a residue corresponding to position 348 in SEQ ID NO: 3;
sssssssssssss) S, G, D, N, T at a residue corresponding to position 349 in SEQ ID NO: 3;
ttttttttttttt) V, E, I, L, F at a residue corresponding to position 350 in SEQ ID NO: 3;
uuuuuuuuuuuuu) D at a residue corresponding to position 351 in SEQ ID NO: 3;
vvvvvvvvvvvvv) Q, H, E at a residue corresponding to position 352 in SEQ ID NO: 3;
wwwwwwwwwwwww) L, V, C at a residue corresponding to position 354 in SEQ ID NO: 3;
xxxxxxxxxxxxx) R, A, D, Q, S, E at a residue corresponding to position 355 in SEQ ID NO: 3;
yyyyyyyyyyyyy) M, L, V at a residue corresponding to position 356 in SEQ ID NO: 3;
zzzzzzzzzzzzz) C at a residue corresponding to position 357 in SEQ ID NO: 3;
aaaaaaaaaaaaaa) N, R at a residue corresponding to position 358 in SEQ ID NO: 3;
bbbbbbbbbbbbbb) A, D, S, N, E at a residue corresponding to position 359 in SEQ ID NO: 3;
cccccccccccccc) S, T at a residue corresponding to position 360 in SEQ ID NO: 3;
dddddddddddddd) D, V at a residue corresponding to position 361 in SEQ ID NO: 3;
eeeeeeeeeeeeee) L, T, N, S at a residue corresponding to position 362 in SEQ ID NO: 3;
ffffffffffffff) Y, E at a residue corresponding to position 363 in SEQ ID NO: 3;
gggggggggggggg) Q, Y, F, H at a residue corresponding to position 364 in SEQ ID NO: 3;
hhhhhhhhhhhhhh) Y, K, F, A, L at a residue corresponding to position 366 in SEQ ID NO: 3;
iiiiiiiiiiiiii) L, A at a residue corresponding to position 368 in SEQ ID NO: 3;
jjjjjjjjjjjjjj) T, A at a residue corresponding to position 369 in SEQ ID NO: 3;
kkkkkkkkkkkkkk) C, A at a residue corresponding to position 370 in SEQ ID NO: 3;
llllllllllllll) M, I, A, W, S, Y at a residue corresponding to position 371 in SEQ ID NO: 3;
mmmmmmmmmmmmmm) V, M at a residue corresponding to position 372 in SEQ ID NO: 3;
nnnnnnnnnnnnnn) W, Y, F at a residue corresponding to position 373 in SEQ ID NO: 3;
oooooooooooooo) T at a residue corresponding to position 374 in SEQ ID NO: 3;
pppppppppppppp) H, Q, K, T, R at a residue corresponding to position 375 in SEQ ID NO: 3;
qqqqqqqqqqqqqq) N, S, D at a residue corresponding to position 376 in SEQ ID NO: 3;
rrrrrrrrrrrrrr) D, I, V, E at a residue corresponding to position 377 in SEQ ID NO: 3;
ssssssssssssss) K, D, A at a residue corresponding to position 378 in SEQ ID NO: 3;
tttttttttttttt) N, K, V, R at a residue corresponding to position 379 in SEQ ID NO: 3;
uuuuuuuuuuuuuu) A, M, V at a residue corresponding to position 380 in SEQ ID NO: 3;
vvvvvvvvvvvvvv) M, W, K, R, T, H at a residue corresponding to position 381 in SEQ ID NO: 3;
wwwwwwwwwwwwww) K, R, E, Q at a residue corresponding to position 382 in SEQ ID NO: 3;
xxxxxxxxxxxxxx) M, L, Y, F, I at a residue corresponding to position 383 in SEQ ID NO: 3;
yyyyyyyyyyyyyy) S, G at a residue corresponding to position 384 in SEQ ID NO: 3;
zzzzzzzzzzzzzz) Q, L, R, K at a residue corresponding to position 385 in SEQ ID NO: 3;
aaaaaaaaaaaaaaa) E, Q, K, R at a residue corresponding to position 386 in SEQ ID NO: 3;
bbbbbbbbbbbbbbb) M, V, L at a residue corresponding to position 387 in SEQ ID NO: 3;
ccccccccccccccc) R, H, D, Q, E at a residue corresponding to position 388 in SEQ ID NO: 3;
ddddddddddddddd) A, C, T at a residue corresponding to position 389 in SEQ ID NO: 3;
eeeeeeeeeeeeeee) T, N, C, G at a residue corresponding to position 390 in SEQ ID NO: 3;
fffffffffffffff) M, T, I at a residue corresponding to position 391 in SEQ ID NO: 3;
ggggggggggggggg) V, I, M at a residue corresponding to position 392 in SEQ ID NO: 3;
hhhhhhhhhhhhhhh) M, I, W, L, G at a residue corresponding to position 393 in SEQ ID NO: 3;
iiiiiiiiiiiiiii) I, K at a residue corresponding to position 394 in SEQ ID NO: 3;
jjjjjjjjjjjjjjj) R, E, S, D at a residue corresponding to position 396 in SEQ ID NO: 3;
kkkkkkkkkkkkkkk) I, W, A, P, F, Y at a residue corresponding to position 397 in SEQ ID NO: 3;
lllllllllllllll) S, C, P, T at a residue corresponding to position 398 in SEQ ID NO: 3;
mmmmmmmmmmmmmmm) A, S at a residue corresponding to position 399 in SEQ ID NO: 3;
nnnnnnnnnnnnnnn) I, S, F, V, H at a residue corresponding to position 400 in SEQ ID NO: 3;
ooooooooooooooo) G, H, D, S, A at a residue corresponding to position 401 in SEQ ID NO: 3;
ppppppppppppppp) N, A, F, M at a residue corresponding to position 402 in SEQ ID NO: 3;
qqqqqqqqqqqqqqq) S at a residue corresponding to position 403 in SEQ ID NO: 3;
rrrrrrrrrrrrrrr) I, F, V, G at a residue corresponding to position 405 in SEQ ID NO: 3;
sssssssssssssss) Y, K, F, L, W at a residue corresponding to position 406 in SEQ ID NO: 3;
ttttttttttttttt) H, Q, Y, E at a residue corresponding to position 407 in SEQ ID NO: 3;
uuuuuuuuuuuuuuu) T, S, H at a residue corresponding to position 408 in SEQ ID NO: 3;
vvvvvvvvvvvvvvv) F, I, M at a residue corresponding to position 410 in SEQ ID NO: 3;
wwwwwwwwwwwwwww) T, G, P, A at a residue corresponding to position 411 in SEQ ID NO: 3;
xxxxxxxxxxxxxxx) F, R at a residue corresponding to position 412 in SEQ ID NO: 3;
yyyyyyyyyyyyyyy) A at a residue corresponding to position 413 in SEQ ID NO: 3;
zzzzzzzzzzzzzzz) M, F, V at a residue corresponding to position 414 in SEQ ID NO: 3;
aaaaaaaaaaaaaaaa) P, S at a residue corresponding to position 415 in SEQ ID NO: 3;
bbbbbbbbbbbbbbbb) M, F, I at a residue corresponding to position 416 in SEQ ID NO: 3;
cccccccccccccccc) F, I, T at a residue corresponding to position 418 in SEQ ID NO: 3;
dddddddddddddddd) S, R at a residue corresponding to position 420 in SEQ ID NO: 3;
eeeeeeeeeeeeeeee) Y, F at a residue corresponding to position 421 in SEQ ID NO: 3;
ffffffffffffffff) K, R, G at a residue corresponding to position 423 in SEQ ID NO: 3;
gggggggggggggggg) S, R at a residue corresponding to position 424 in SEQ ID NO: 3;
hhhhhhhhhhhhhhhh) I, G, W, A at a residue corresponding to position 425 in SEQ ID NO: 3;
iiiiiiiiiiiiiiii) V, T, L at a residue corresponding to position 426 in SEQ ID NO: 3;
jjjjjjjjjjjjjjjj) G, T, A at a residue corresponding to position 427 in SEQ ID NO: 3;
kkkkkkkkkkkkkkkk) D, H at a residue corresponding to position 428 in SEQ ID NO: 3;
llllllllllllllll) E at a residue corresponding to position 429 in SEQ ID NO: 3;
mmmmmmmmmmmmmmmm) I, Y, L, V at a residue corresponding to position 430 in SEQ ID NO: 3;
nnnnnnnnnnnnnnnn) G, S, N at a residue corresponding to position 431 in SEQ ID NO: 3;
oooooooooooooooo) A, P, F, W at a residue corresponding to position 432 in SEQ ID NO: 3;
pppppppppppppppp) T, R, K, H, M, E at a residue corresponding to position 433 in SEQ ID NO: 3;
qqqqqqqqqqqqqqqq) R, Q, D, N at a residue corresponding to position 434 in SEQ ID NO: 3;
rrrrrrrrrrrrrrrr) V, F, W, Y, M at a residue corresponding to position 435 in SEQ ID NO: 3;
ssssssssssssssss) T, L, F, I, V at a residue corresponding to position 436 in SEQ ID NO: 3;
tttttttttttttttt) H, Q, Y at a residue corresponding to position 437 in SEQ ID NO: 3;
uuuuuuuuuuuuuuuu) V, T, W, C, Y, I at a residue corresponding to position 438 in SEQ ID NO: 3;
vvvvvvvvvvvvvvvv) Q, S, R, K at a residue corresponding to position 439 in SEQ ID NO: 3;
wwwwwwwwwwwwwwww) K, W at a residue corresponding to position 440 in SEQ ID NO: 3;
xxxxxxxxxxxxxxxx) L, I, M at a residue corresponding to position 441 in SEQ ID NO: 3;
yyyyyyyyyyyyyyyy) G, A at a residue corresponding to position 442 in SEQ ID NO: 3;
zzzzzzzzzzzzzzzz) H, W, R, F at a residue corresponding to position 443 in SEQ ID NO: 3;
aaaaaaaaaaaaaaaaa) E, P, K at a residue corresponding to position 444 in SEQ ID NO: 3;
bbbbbbbbbbbbbbbbb) I, Y, F, M, L at a residue corresponding to position 445 in SEQ ID NO: 3;
ccccccccccccccccc) E, K at a residue corresponding to position 446 in SEQ ID NO: 3;
ddddddddddddddddd) E, N at a residue corresponding to position 447 in SEQ ID NO: 3;
eeeeeeeeeeeeeeeee) W, K, P at a residue corresponding to position 448 in SEQ ID NO: 3;
fffffffffffffffff) E, R, F, H at a residue corresponding to position 449 in SEQ ID NO: 3;
ggggggggggggggggg) M, C, F, V, L at a residue corresponding to position 450 in SEQ ID NO: 3;
hhhhhhhhhhhhhhhhh) W at a residue corresponding to position 451 in SEQ ID NO: 3;
iiiiiiiiiiiiiiiii) S, D, K at a residue corresponding to position 452 in SEQ ID NO: 3;
jjjjjjjjjjjjjjjjj) E, P, H at a residue corresponding to position 453 in SEQ ID NO: 3;
kkkkkkkkkkkkkkkkk) A, D at a residue corresponding to position 454 in SEQ ID NO: 3;
lllllllllllllllll) K, Q, D at a residue corresponding to position 455 in SEQ ID NO: 3;
mmmmmmmmmmmmmmmmm) F, L, V, W, M at a residue corresponding to position 456 in SEQ ID NO: 3;
nnnnnnnnnnnnnnnnn) M, Y, F at a residue corresponding to position 457 in SEQ ID NO: 3;
ooooooooooooooooo) H, G at a residue corresponding to position 458 in SEQ ID NO: 3;
ppppppppppppppppp) D at a residue corresponding to position 459 in SEQ ID NO: 3; or
qqqqqqqqqqqqqqqqq) Any combination thereof.

52. The engineered ALDH of claim 51, wherein the one or more amino acid alterations result in an engineered ALDH comprising:
a) Q, T, P at a residue corresponding to position 2 in SEQ ID NO: 3;
b) S, K at a residue corresponding to position 3 in SEQ ID NO: 3;
c) G, S at a residue corresponding to position 4 in SEQ ID NO: 3;
d) I, T at a residue corresponding to position 5 in SEQ ID NO: 3;
e) F at a residue corresponding to position 6 in SEQ ID NO: 3;
f) E, Q at a residue corresponding to position 7 in SEQ ID NO: 3;
g) V at a residue corresponding to position 9 in SEQ ID NO: 3;
h) E, D, Q, R at a residue corresponding to position 11 in SEQ ID NO: 3;
i) L, A at a residue corresponding to position 12 in SEQ ID NO: 3;
j) R, K at a residue corresponding to position 15 in SEQ ID NO: 3;
k) A at a residue corresponding to position 16 in SEQ ID NO: 3;
l) K at a residue corresponding to position 17 in SEQ ID NO: 3;
m) K, E, Q at a residue corresponding to position 18 in SEQ ID NO: 3;
n) F, H at a residue corresponding to position 20 in SEQ ID NO: 3;
o) A, R at a residue corresponding to position 21 in SEQ ID NO: 3;
p) K, Q, A at a residue corresponding to position 22 in SEQ ID NO: 3;
q) Y at a residue corresponding to position 23 in SEQ ID NO: 3;
r) E, K at a residue corresponding to position 24 in SEQ ID NO: 3;
s) A, E at a residue corresponding to position 25 in SEQ ID NO: 3;
t) L, M, W at a residue corresponding to position 26 in SEQ ID NO: 3;
u) D, T at a residue corresponding to position 27 in SEQ ID NO: 3;
v) W at a residue corresponding to position 28 in SEQ ID NO: 3;
w) D, K, Q at a residue corresponding to position 30 in SEQ ID NO: 3;
x) I, L at a residue corresponding to position 31 in SEQ ID NO: 3;
y) N, E at a residue corresponding to position 32 in SEQ ID NO: 3;
z) E, Q, A, R at a residue corresponding to position 33 in SEQ ID NO: 3;
aa) V at a residue corresponding to position 34 in SEQ ID NO: 3;
bb) C, M at a residue corresponding to position 35 in SEQ ID NO: 3;
cc) K at a residue corresponding to position 36 in SEQ ID NO: 3;
dd) K, A, R at a residue corresponding to position 37 in SEQ ID NO: 3;
ee) L, I at a residue corresponding to position 38 in SEQ ID NO: 3;
ff) A at a residue corresponding to position 39 in SEQ ID NO: 3;
gg) W at a residue corresponding to position 40 in SEQ ID NO: 3;
hh) L, V at a residue corresponding to position 41 in SEQ ID NO: 3;
ii) L, V, M, C at a residue corresponding to position 42 in SEQ ID NO: 3;
jj) E, L at a residue corresponding to position 43 in SEQ ID NO: 3;
kk) K, Q at a residue corresponding to position 44 in SEQ ID NO: 3;
ll) H, R at a residue corresponding to position 45 in SEQ ID NO: 3;
mm) D at a residue corresponding to position 47 in SEQ ID NO: 3;
nn) E, M, D at a residue corresponding to position 48 in SEQ ID NO: 3;
oo) I at a residue corresponding to position 49 in SEQ ID NO: 3;
pp) N, E, Q at a residue corresponding to position 51 in SEQ ID NO: 3;
qq) L at a residue corresponding to position 52 in SEQ ID NO: 3;
rr) L at a residue corresponding to position 54 in SEQ ID NO: 3;
ss) E, R at a residue corresponding to position 55 in SEQ ID NO: 3;
tt) R at a residue corresponding to position 58 in SEQ ID NO: 3;
uu) D, E, R at a residue corresponding to position 61 in SEQ ID NO: 3;
vv) F, V, W at a residue corresponding to position 62 in SEQ ID NO: 3;
ww) D, E, Q at a residue corresponding to position 63 in SEQ ID NO: 3;
xx) H at a residue corresponding to position 64 in SEQ ID NO: 3;
yy) R at a residue corresponding to position 65 in SEQ ID NO: 3;
zz) V at a residue corresponding to position 66 in SEQ ID NO: 3;
aaa) E at a residue corresponding to position 67 in SEQ ID NO: 3;
bbb) M, R at a residue corresponding to position 70 in SEQ ID NO: 3;
ccc) D, E, Y, F, H at a residue corresponding to position 71 in SEQ ID NO: 3;
ddd) T at a residue corresponding to position 73 in SEQ ID NO: 3;
eee) E, R at a residue corresponding to position 74 in SEQ ID NO: 3;
fff) G at a residue corresponding to position 75 in SEQ ID NO: 3;
ggg) Y at a residue corresponding to position 77 in SEQ ID NO: 3;
hhh) E, F, Y, H, N at a residue corresponding to position 78 in SEQ ID NO: 3;
iii) D, W at a residue corresponding to position 79 in SEQ ID NO: 3;
jjj) F, V, I at a residue corresponding to position 80 in SEQ ID NO: 3;
kkk) A, R at a residue corresponding to position 81 in SEQ ID NO: 3;
lll) G at a residue corresponding to position 82 in SEQ ID NO: 3;
mmm) E at a residue corresponding to position 83 in SEQ ID NO: 3;
nnn) R, Q at a residue corresponding to position 84 in SEQ ID NO: 3;
ooo) V, K at a residue corresponding to position 86 in SEQ ID NO: 3;
ppp) M at a residue corresponding to position 88 in SEQ ID NO: 3;
qqq) V, L at a residue corresponding to position 89 in SEQ ID NO: 3;
rrr) S, D, H, E, R at a residue corresponding to position 90 in SEQ ID NO: 3;
sss) E, Q at a residue corresponding to position 91 in SEQ ID NO: 3;
ttt) D at a residue corresponding to position 92 in SEQ ID NO: 3;
uuu) D at a residue corresponding to position 93 in SEQ ID NO: 3;
vvv) G, D at a residue corresponding to position 94 in SEQ ID NO: 3;
www) K, M, R at a residue corresponding to position 95 in SEQ ID NO: 3;
xxx) N at a residue corresponding to position 96 in SEQ ID NO: 3;
yyy) I, V, M at a residue corresponding to position 97 in SEQ ID NO: 3;
zzz) Y, I, M, T at a residue corresponding to position 98 in SEQ ID NO: 3;
aaaa) R, T, Q, K at a residue corresponding to position 99 in SEQ ID NO: 3;
bbbb) Y, I, M, L at a residue corresponding to position 100 in SEQ ID NO: 3;
cccc) Y, H at a residue corresponding to position 101 in SEQ ID NO: 3;
dddd) R, Q at a residue corresponding to position 102 in SEQ ID NO: 3;
eeee) F, M at a residue corresponding to position 104 in SEQ ID NO: 3;
ffff) C, V at a residue corresponding to position 107 in SEQ ID NO: 3;
gggg) T, S at a residue corresponding to position 111 in SEQ ID NO: 3;
hhhh) I, T, L at a residue corresponding to position 117 in SEQ ID NO: 3;
iiii) T, S at a residue corresponding to position 119 in SEQ ID NO: 3;
jjjj) A, T, M at a residue corresponding to position 120 in SEQ ID NO: 3;
kkkk) L, V, I at a residue corresponding to position 121 in SEQ ID NO: 3;
llll) H at a residue corresponding to position 122 in SEQ ID NO: 3;
mmmm) I, V, L, M at a residue corresponding to position 124 in SEQ ID NO: 3;
nnnn) I, L, V at a residue corresponding to position 125 in SEQ ID NO: 3;
oooo) A at a residue corresponding to position 126 in SEQ ID NO: 3;
pppp) I at a residue corresponding to position 128 in SEQ ID NO: 3;
qqqq) A, M at a residue corresponding to position 129 in SEQ ID NO: 3;
rrrr) A, S at a residue corresponding to position 130 in SEQ ID NO: 3;
ssss) C, T at a residue corresponding to position 133 in SEQ ID NO: 3;
tttt) L, I at a residue corresponding to position 136 in SEQ ID NO: 3;
uuuu) R at a residue corresponding to position 144 in SEQ ID NO: 3;
vvvv) C, T, V at a residue corresponding to position 145 in SEQ ID NO: 3;
wwww) N at a residue corresponding to position 146 in SEQ ID NO: 3;
xxxx) A, I, M at a residue corresponding to position 147 in SEQ ID NO: 3;
yyyy) E, L, A, R at a residue corresponding to position 148 in SEQ ID NO: 3;
zzzz) M, V, L, I at a residue corresponding to position 149 in SEQ ID NO: 3;
aaaaa) A, I, M at a residue corresponding to position 150 in SEQ ID NO: 3;
bbbbb) K, D, Q at a residue corresponding to position 151 in SEQ ID NO: 3;
ccccc) M, I at a residue corresponding to position 152 in SEQ ID NO: 3;
ddddd) M, C, F, V at a residue corresponding to position 153 in SEQ ID NO: 3;
eeeee) Q, K at a residue corresponding to position 154 in SEQ ID NO: 3;
fffff) K, R, D, Q at a residue corresponding to position 155 in SEQ ID NO: 3;
ggggg) I at a residue corresponding to position 156 in SEQ ID NO: 3;
hhhhh) A, F, I at a residue corresponding to position 157 in SEQ ID NO: 3;
iiiii) E, V, K at a residue corresponding to position 158 in SEQ ID NO: 3;
jjjjj) D, K at a residue corresponding to position 159 in SEQ ID NO: 3;
kkkkk) V, A at a residue corresponding to position 160 in SEQ ID NO: 3;
lllll) F, Y, V, A, L at a residue corresponding to position 162 in SEQ ID NO: 3;
mmmmm) A, D, P at a residue corresponding to position 164 in SEQ ID NO: 3;
nnnnn) D, H at a residue corresponding to position 165 in SEQ ID NO: 3;
ooooo) V, C, I at a residue corresponding to position 166 in SEQ ID NO: 3;
ppppp) L, V, F at a residue corresponding to position 167 in SEQ ID NO: 3;
qqqqq) T, N at a residue corresponding to position 168 in SEQ ID NO: 3;
rrrrr) C, L, M, I at a residue corresponding to position 169 in SEQ ID NO: 3;
sssss) V at a residue corresponding to position 170 in SEQ ID NO: 3;
ttttt) D, E, P, N at a residue corresponding to position 171 in SEQ ID NO: 3;
uuuuu) D, G, H at a residue corresponding to position 172 in SEQ ID NO: 3;
vvvvv) T at a residue corresponding to position 174 in SEQ ID NO: 3;
wwwww) I, H at a residue corresponding to position 175 in SEQ ID NO: 3;
xxxxx) D at a residue corresponding to position 176 in SEQ ID NO: 3;
yyyyy) A, T, I at a residue corresponding to position 177 in SEQ ID NO: 3;
zzzzz) E at a residue corresponding to position 180 in SEQ ID NO: 3;
aaaaaa) I at a residue corresponding to position 181 in SEQ ID NO: 3;
bbbbbb) Q, A, H, K at a residue corresponding to position 183 in SEQ ID NO: 3;
cccccc) G, S at a residue corresponding to position 184 in SEQ ID NO: 3;
dddddd) M, V, I, F, K at a residue corresponding to position 187 in SEQ ID NO: 3;
eeeeee) V, L at a residue corresponding to position 189 in SEQ ID NO: 3;
ffffff) A at a residue corresponding to position 190 in SEQ ID NO: 3;
gggggg) A, T, R, K at a residue corresponding to position 194 in SEQ ID NO: 3;
hhhhhh) V at a residue corresponding to position 196 in SEQ ID NO: 3;
iiiiii) I at a residue corresponding to position 197 in SEQ ID NO: 3;
jjjjjj) R at a residue corresponding to position 198 in SEQ ID NO: 3;
kkkkkk) V at a residue corresponding to position 199 in SEQ ID NO: 3;
llllll) C at a residue corresponding to position 200 in SEQ ID NO: 3;
mmmmmm) A at a residue corresponding to position 201 in SEQ ID NO: 3;
nnnnnn) A, T, R at a residue corresponding to position 202 in SEQ ID NO: 3;
oooooo) N, H, A at a residue corresponding to position 203 in SEQ ID NO: 3;
pppppp) V at a residue corresponding to position 206 in SEQ ID NO: 3;
qqqqqq) V, T, C at a residue corresponding to position 207 in SEQ ID NO: 3;
rrrrrr) V, M, Y at a residue corresponding to position 208 in SEQ ID NO: 3;
ssssss) A, P at a residue corresponding to position 212 in SEQ ID NO: 3;
tttttt) S at a residue corresponding to position 214 in SEQ ID NO: 3;
uuuuuu) P, N, C, A, S at a residue corresponding to position 215 in SEQ ID NO: 3;
vvvvvv) A, P at a residue corresponding to position 216 in SEQ ID NO: 3;
wwwwww) M, A, C at a residue corresponding to position 217 in SEQ ID NO: 3;
xxxxxx) I, V at a residue corresponding to position 218 in SEQ ID NO: 3;
yyyyyy) I, M, L at a residue corresponding to position 219 in SEQ ID NO: 3;
zzzzzz) P, D at a residue corresponding to position 221 in SEQ ID NO: 3;
aaaaaaa) T, S at a residue corresponding to position 222 in SEQ ID NO: 3;
bbbbbbb) C at a residue corresponding to position 223 in SEQ ID NO: 3;
ccccccc) N at a residue corresponding to position 224 in SEQ ID NO: 3;
ddddddd) V at a residue corresponding to position 225 in SEQ ID NO: 3;
eeeeeee) M, E, K at a residue corresponding to position 227 in SEQ ID NO: 3;
fffffff) A at a residue corresponding to position 228 in SEQ ID NO: 3;
ggggggg) V at a residue corresponding to position 229 in SEQ ID NO: 3;
hhhhhhh) D, E, P, K at a residue corresponding to position 230 in SEQ ID NO: 3;
iiiiiii) R, Q, I, K at a residue corresponding to position 231 in SEQ ID NO: 3;
jjjjjjj) M at a residue corresponding to position 232 in SEQ ID NO: 3;
kkkkkkk) L at a residue corresponding to position 233 in SEQ ID NO: 3;
lllllll) F, A, W, M, Q at a residue corresponding to position 234 in SEQ ID NO: 3;
mmmmmmm) F, Y, C, M at a residue corresponding to position 237 in SEQ ID NO: 3;
nnnnnnn) R, Q, M at a residue corresponding to position 238 in SEQ ID NO: 3;
ooooooo) T at a residue corresponding to position 240 in SEQ ID NO: 3;
ppppppp) V at a residue corresponding to position 243 in SEQ ID NO: 3;
qqqqqqq) I at a residue corresponding to position 245 in SEQ ID NO: 3;
rrrrrrr) S, A at a residue corresponding to position 246 in SEQ ID NO: 3;
sssssss) N at a residue corresponding to position 248 in SEQ ID NO: 3;
ttttttt) Y at a residue corresponding to position 249 in SEQ ID NO: 3;
uuuuuuu) M at a residue corresponding to position 250 in SEQ ID NO: 3;
vvvvvvv) F, L at a residue corresponding to position 251 in SEQ ID NO: 3;
wwwwwww) V, I at a residue corresponding to position 252 in SEQ ID NO: 3;
xxxxxxx) H at a residue corresponding to position 253 in SEQ ID NO: 3;
yyyyyyy) K, R, D at a residue corresponding to position 254 in SEQ ID NO: 3;
zzzzzzz) E at a residue corresponding to position 255 in SEQ ID NO: 3;
aaaaaaaa) I, M, V, R at a residue corresponding to position 256 in SEQ ID NO: 3;
bbbbbbbb) Y, A at a residue corresponding to position 257 in SEQ ID NO: 3;
cccccccc) D at a residue corresponding to position 258 in SEQ ID NO: 3;
dddddddd) R, A, Q, D at a residue corresponding to position 259 in SEQ ID NO: 3;
eeeeeeee) V, L at a residue corresponding to position 260 in SEQ ID NO: 3;
ffffffff) V, L, M at a residue corresponding to position 261 in SEQ ID NO: 3;
gggggggg) K at a residue corresponding to position 262 in SEQ ID NO: 3;
hhhhhhhh) K, R at a residue corresponding to position 263 in SEQ ID NO: 3;
iiiiiiii) V, F at a residue corresponding to position 264 in SEQ ID NO: 3;
jjjjjjjj) R, Q, E at a residue corresponding to position 265 in SEQ ID NO: 3;
kkkkkkkk) E at a residue corresponding to position 266 in SEQ ID NO: 3;
llllllll) R at a residue corresponding to position 267 in SEQ ID NO: 3;
mmmmmmmm) F, L, W, M, I at a residue corresponding to position 270 in SEQ ID NO: 3;
nnnnnnnn) W, F, K, H at a residue corresponding to position 271 in SEQ ID NO: 3;
oooooooo) V, L, M at a residue corresponding to position 272 in SEQ ID NO: 3;
pppppppp) C at a residue corresponding to position 273 in SEQ ID NO: 3;
qqqqqqqq) D, E at a residue corresponding to position 274 in SEQ ID NO: 3;
rrrrrrrr) A, P, G, E at a residue corresponding to position 275 in SEQ ID NO: 3;
ssssssss) R, D at a residue corresponding to position 276 in SEQ ID NO: 3;
tttttttt) E, Q at a residue corresponding to position 277 in SEQ ID NO: 3;
uuuuuuuu) E at a residue corresponding to position 278 in SEQ ID NO: 3;
vvvvvvvv) S, E at a residue corresponding to position 279 in SEQ ID NO: 3;
wwwwwwww) T at a residue corresponding to position 280 in SEQ ID NO: 3;
xxxxxxxx) I, V, M at a residue corresponding to position 281 in SEQ ID NO: 3;
yyyyyyyy) K, Q, G, M at a residue corresponding to position 282 in SEQ ID NO: 3;
zzzzzzzz) E, N, H at a residue corresponding to position 283 in SEQ ID NO: 3;
aaaaaaaaa) V at a residue corresponding to position 284 in SEQ ID NO: 3;
bbbbbbbbb) V at a residue corresponding to position 285 in SEQ ID NO: 3;
ccccccccc) Y, V, I at a residue corresponding to position 286 in SEQ ID NO: 3;
ddddddddd) E at a residue corresponding to position 288 in SEQ ID NO: 3;
eeeeeeeee) H at a residue corresponding to position 290 in SEQ ID NO: 3;
fffffffff) L, A, T, H, N, K at a residue corresponding to position 291 in SEQ ID NO: 3;
ggggggggg) A, I, N at a residue corresponding to position 293 in SEQ ID NO: 3;
hhhhhhhhh) R at a residue corresponding to position 294 in SEQ ID NO: 3;
iiiiiiiii) D, R, Q at a residue corresponding to position 295 in SEQ ID NO: 3;
jjjjjjjjj) L, W at a residue corresponding to position 296 in SEQ ID NO: 3;
kkkkkkkkk) K, Q, R at a residue corresponding to position 299 in SEQ ID NO: 3;
lllllllll) A at a residue corresponding to position 300 in SEQ ID NO: 3;
mmmmmmmmm) A at a residue corresponding to position 301 in SEQ ID NO: 3;
nnnnnnnnn) A, E, Q at a residue corresponding to position 302 in SEQ ID NO: 3;
ooooooooo) E, R at a residue corresponding to position 303 in SEQ ID NO: 3;
ppppppppp) L at a residue corresponding to position 304 in SEQ ID NO: 3;
qqqqqqqqq) V at a residue corresponding to position 305 in SEQ ID NO: 3;
rrrrrrrrr) E, H, K, R at a residue corresponding to position 306 in SEQ ID NO: 3;
sssssssss) I, M, V at a residue corresponding to position 307 in SEQ ID NO: 3;
ttttttttt) T at a residue corresponding to position 308 in SEQ ID NO: 3;
uuuuuuuuu) F, V, L at a residue corresponding to position 310 in SEQ ID NO: 3;
vvvvvvvvv) D, S, R, E at a residue corresponding to position 311 in SEQ ID NO: 3;
wwwwwwwww) A, T, D, K at a residue corresponding to position 312 in SEQ ID NO: 3;
xxxxxxxxx) V, L at a residue corresponding to position 313 in SEQ ID NO: 3;
yyyyyyyyy) D at a residue corresponding to position 315 in SEQ ID NO: 3;
zzzzzzzzz) G, E, N at a residue corresponding to position 316 in SEQ ID NO: 3;
aaaaaaaaaa) G, T at a residue corresponding to position 317 in SEQ ID NO: 3;
bbbbbbbbbb) R at a residue corresponding to position 318 in SEQ ID NO: 3;
cccccccccc) L, M at a residue corresponding to position 319 in SEQ ID NO: 3;
dddddddddd) Y, L at a residue corresponding to position 320 in SEQ ID NO: 3;
eeeeeeeeee) I, L, C at a residue corresponding to position 321 in SEQ ID NO: 3;
ffffffffff) G, V, A, T at a residue corresponding to position 322 in SEQ ID NO: 3;
gggggggggg) I, V, K, P at a residue corresponding to position 323 in SEQ ID NO: 3;
hhhhhhhhhh) I, V, L at a residue corresponding to position 324 in SEQ ID NO: 3;
iiiiiiiiii) E, T at a residue corresponding to position 325 in SEQ ID NO: 3;
jjjjjjjjjj) D at a residue corresponding to position 326 in SEQ ID NO: 3;
kkkkkkkkkk) F, W, V at a residue corresponding to position 327 in SEQ ID NO: 3;
llllllllll) S at a residue corresponding to position 328 in SEQ ID NO: 3;
mmmmmmmmmm) P, R, D, N at a residue corresponding to position 329 in SEQ ID NO: 3;
nnnnnnnnnn) E at a residue corresponding to position 330 in SEQ ID NO: 3;
oooooooooo) D at a residue corresponding to position 331 in SEQ ID NO: 3;
pppppppppp) T, R at a residue corresponding to position 332 in SEQ ID NO: 3;
qqqqqqqqqq) I, M, F at a residue corresponding to position 333 in SEQ ID NO: 3;
rrrrrrrrrr) S, V, C, M at a residue corresponding to position 334 in SEQ ID NO: 3;
ssssssssss) R at a residue corresponding to position 335 in SEQ ID NO: 3;
tttttttttt) M at a residue corresponding to position 339 in SEQ ID NO: 3;
uuuuuuuuuu) I at a residue corresponding to position 341 in SEQ ID NO: 3;
vvvvvvvvvv) I, M at a residue corresponding to position 342 in SEQ ID NO: 3;
wwwwwwwwww) S, C, V at a residue corresponding to position 343 in SEQ ID NO: 3;
xxxxxxxxxx) M, I, V, L at a residue corresponding to position 344 in SEQ ID NO: 3;
yyyyyyyyyy) L, M, Y, V at a residue corresponding to position 345 in SEQ ID NO: 3;
zzzzzzzzzz) P at a residue corresponding to position 346 in SEQ ID NO: 3;
aaaaaaaaaaa) F, V at a residue corresponding to position 347 in SEQ ID NO: 3;
bbbbbbbbbbb) E, K, S at a residue corresponding to position 348 in SEQ ID NO: 3;
ccccccccccc) G, T, N, S, D at a residue corresponding to position 349 in SEQ ID NO: 3;
ddddddddddd) E, I, V, F, L at a residue corresponding to position 350 in SEQ ID NO: 3;
eeeeeeeeeee) D at a residue corresponding to position 351 in SEQ ID NO: 3;
fffffffffff) L, C at a residue corresponding to position 354 in SEQ ID NO: 3;
ggggggggggg) D, A, Q, S, E at a residue corresponding to position 355 in SEQ ID NO: 3;
hhhhhhhhhhh) L, V at a residue corresponding to position 356 in SEQ ID NO: 3;
iiiiiiiiiii) R, N at a residue corresponding to position 358 in SEQ ID NO: 3;
jjjjjjjjjjj) S, A, N, E at a residue corresponding to position 359 in SEQ ID NO: 3;
kkkkkkkkkkk) S at a residue corresponding to position 360 in SEQ ID NO: 3;
lllllllllll) S, N, T at a residue corresponding to position 362 in SEQ ID NO: 3;
mmmmmmmmmmm) E at a residue corresponding to position 363 in SEQ ID NO: 3;
nnnnnnnnnnn) A, K, Y, L at a residue corresponding to position 366 in SEQ ID NO: 3;
ooooooooooo) C, A at a residue corresponding to position 370 in SEQ ID NO: 3;
ppppppppppp) S, A at a residue corresponding to position 371 in SEQ ID NO: 3;
qqqqqqqqqqq) V, M at a residue corresponding to position 372 in SEQ ID NO: 3;
rrrrrrrrrrr) F, Y, W at a residue corresponding to position 373 in SEQ ID NO: 3;
sssssssssss) T at a residue corresponding to position 374 in SEQ ID NO: 3;
ttttttttttt) H, T, K, R, Q at a residue corresponding to position 375 in SEQ ID NO: 3;
uuuuuuuuuuu) D at a residue corresponding to position 376 in SEQ ID NO: 3;
vvvvvvvvvvv) E, I, V at a residue corresponding to position 377 in SEQ ID NO: 3;
wwwwwwwwwww) K, A, D at a residue corresponding to position 378 in SEQ ID NO: 3;
xxxxxxxxxxx) R, N, V at a residue corresponding to position 379 in SEQ ID NO: 3;
yyyyyyyyyyy) V at a residue corresponding to position 380 in SEQ ID NO: 3;
zzzzzzzzzzz) W, H, M, R, K, T at a residue corresponding to position 381 in SEQ ID NO: 3;
aaaaaaaaaaaa) K at a residue corresponding to position 382 in SEQ ID NO: 3;
bbbbbbbbbbbb) I, M at a residue corresponding to position 383 in SEQ ID NO: 3;
cccccccccccc) G, S at a residue corresponding to position 384 in SEQ ID NO: 3;
dddddddddddd) R, K at a residue corresponding to position 385 in SEQ ID NO: 3;
eeeeeeeeeeee) Q, K, R at a residue corresponding to position 386 in SEQ ID NO: 3;
ffffffffffff) M, V, L at a residue corresponding to position 387 in SEQ ID NO: 3;
gggggggggggg) R, E, Q, H, D at a residue corresponding to position 388 in SEQ ID NO: 3;
hhhhhhhhhhhh) T, A, C at a residue corresponding to position 389 in SEQ ID NO: 3;
iiiiiiiiiiii) G, T, C at a residue corresponding to position 390 in SEQ ID NO: 3;
jjjjjjjjjjjj) M, V, I at a residue corresponding to position 392 in SEQ ID NO: 3;
kkkkkkkkkkkk) G at a residue corresponding to position 393 in SEQ ID NO: 3;
llllllllllll) I at a residue corresponding to position 394 in SEQ ID NO: 3;
mmmmmmmmmmmm) E, D, S at a residue corresponding to position 396 in SEQ ID NO: 3;
nnnnnnnnnnnn) W, I, Y at a residue corresponding to position 397 in SEQ ID NO: 3;
oooooooooooo) S, I, H at a residue corresponding to position 400 in SEQ ID NO: 3;
pppppppppppp) D, G, S, H, A at a residue corresponding to position 401 in SEQ ID NO: 3;
qqqqqqqqqqqq) M at a residue corresponding to position 402 in SEQ ID NO: 3;
rrrrrrrrrrrr) V, G at a residue corresponding to position 405 in SEQ ID NO: 3;
ssssssssssss) W, F, L at a residue corresponding to position 406 in SEQ ID NO: 3;
tttttttttttt) H, Q at a residue corresponding to position 407 in SEQ ID NO: 3;
uuuuuuuuuuuu) M at a residue corresponding to position 410 in SEQ ID NO: 3;
vvvvvvvvvvvv) G, A, P, T at a residue corresponding to position 411 in SEQ ID NO: 3;
wwwwwwwwwwww) V at a residue corresponding to position 414 in SEQ ID NO: 3;
xxxxxxxxxxxx) S at a residue corresponding to position 420 in SEQ ID NO: 3;
yyyyyyyyyyyy) F at a residue corresponding to position 421 in SEQ ID NO: 3;
zzzzzzzzzzzz) G, R at a residue corresponding to position 423 in SEQ ID NO: 3;
aaaaaaaaaaaaa) I, A, G at a residue corresponding to position 425 in SEQ ID NO: 3;
bbbbbbbbbbbbb) V at a residue corresponding to position 426 in SEQ ID NO: 3;
ccccccccccccc) A, T at a residue corresponding to position 427 in SEQ ID NO: 3;
ddddddddddddd) D at a residue corresponding to position 428 in SEQ ID NO: 3;
eeeeeeeeeeeee) N at a residue corresponding to position 431 in SEQ ID NO: 3;
fffffffffffff) T, H, K, M, R at a residue corresponding to position 433 in SEQ ID NO: 3;
ggggggggggggg) M, V at a residue corresponding to position 435 in SEQ ID NO: 3;
hhhhhhhhhhhhh) V, F, I at a residue corresponding to position 436 in SEQ ID NO: 3;
iiiiiiiiiiiii) Q at a residue corresponding to position 437 in SEQ ID NO: 3;
jjjjjjjjjjjjj) I, T, Y, C at a residue corresponding to position 438 in SEQ ID NO: 3;
kkkkkkkkkkkkk) S at a residue corresponding to position 439 in SEQ ID NO: 3;
lllllllllllll) W, K at a residue corresponding to position 440 in SEQ ID NO: 3;
mmmmmmmmmmmmm) M, I, L at a residue corresponding to position 441 in SEQ ID NO: 3;
nnnnnnnnnnnnn) W, F, R at a residue corresponding to position 443 in SEQ ID NO: 3;
ooooooooooooo) K at a residue corresponding to position 444 in SEQ ID NO: 3;
ppppppppppppp) K at a residue corresponding to position 446 in SEQ ID NO: 3;
qqqqqqqqqqqqq) E, N at a residue corresponding to position 447 in SEQ ID NO: 3;
rrrrrrrrrrrrr) W, K, P at a residue corresponding to position 448 in SEQ ID NO: 3;
sssssssssssss) R, H at a residue corresponding to position 449 in SEQ ID NO: 3;
ttttttttttttt) V at a residue corresponding to position 450 in SEQ ID NO: 3;
uuuuuuuuuuuuu) S, K at a residue corresponding to position 452 in SEQ ID NO: 3;
vvvvvvvvvvvvv) H, P at a residue corresponding to position 453 in SEQ ID NO: 3;
wwwwwwwwwwwww) A, D at a residue corresponding to position 454 in SEQ ID NO: 3;
xxxxxxxxxxxxx) K, D at a residue corresponding to position 455 in SEQ ID NO: 3;
yyyyyyyyyyyyy) F at a residue corresponding to position 457 in SEQ ID NO: 3;
zzzzzzzzzzzzz) H, G at a residue corresponding to position 458 in SEQ ID NO: 3;
aaaaaaaaaaaaaa) D at a residue corresponding to position 459 in SEQ ID NO: 3; or
bbbbbbbbbbbbbb) any combination thereof.

53. The engineered ALDH of any one of claims 50 to 52, wherein the one or more amino acid alterations result in an engineered ALDH comprising:
a) V at a residue corresponding to position 42 in SEQ ID NO: 3;
b) T or S at a residue corresponding to position 119 in SEQ ID NO: 3;
c) I at a residue corresponding to position 149 in SEQ ID NO: 3;
d) K at a residue corresponding to position 158 in SEQ ID NO: 3;
e) T at a residue corresponding to position 174 in SEQ ID NO: 3;
f) A at a residue corresponding to position 246 in SEQ ID NO: 3;
g) D at a residue corresponding to position 259 in SEQ ID NO: 3;
h) R at a residue corresponding to position 263 in SEQ ID NO: 3;
i) R at a residue corresponding to position 299 in SEQ ID NO: 3;
j) V at a residue corresponding to position 327 in SEQ ID NO: 3;
k) S at a residue corresponding to position 348 in SEQ ID NO: 3;
l) A at a residue corresponding to position 370 in SEQ ID NO: 3;
m) I at a residue corresponding to position 392 in SEQ ID NO: 3;
n) A at a residue corresponding to position 401 in SEQ ID NO: 3;
o) T at a residue corresponding to position 427 in SEQ ID NO: 3; or
p) any combination thereof.

54. The engineered ALDH of claims 38 to 48, wherein the engineered ALDH comprises one or more amino acid alterations selected from the group consisting of: E2P, E2Q, E2T, Q3E, Q3K, Q3M, Q3R, Q3S, A4G, A4S, V5I, V5T, K6E, K6F, K6H, K6P, K6Q, K6R, D7E, D7Q, Y8D, L9I, L9V, D10N, K11A, K11D, K11E, K11Q, K11R, M12A, M12I, M12L, V13L, A14D, A14E, A14K, A14Q, A15K, A15R, S16A, R17H, R17K, R17Q, 118E, I18K, I18Q, I18T, Q20F, Q20H, Q20M, Q21A, Q21E, Q21K, Q21P, Q21R, E22A, E22K, E22Q, F23L, F23W, F23Y, A24E, A24K, A24R, A24S, T25A, T25E, T25K, T25N, T25S, Y26F, Y26L, Y26M, Y26T, Y26W, P27D, P27S, P27T, Q28W, E29K, E29Q, T30D, T30E, T30K, T30Q, T30R, V31I, V31L, V31R, D32A, D32E, D32N, K33A, K33E, K33Q, K33R, A34I, A34L, A34M, A34V, V35C, V35I, V35L, V35M, R36E, R36H, R36K, T37A, T37K, T37R, V38F, V38I, V38L, V38M, V38W, G39A, G39R, K40D, K40E, K40Q, K40R, K40W, A41L, A41V, I42C, I42L, I42M, I42V, Y43E, Y43L, Y43R, D44E, D44K, D44Q, D44R, N45H, N45Q, N45R, E47D, L48D, L48E, L48M, L48R, L49F, L49I, L49M, L49W, L49Y, A50G, H51A, H51E, H51K, H51N, H51Q, H51R, M52L, A53E, A53I, A53S, V54I, V54L, V54M, V54T, D55E, D55Q, D55R, K58E, K58G, K58Q, K58R, M59F, M59I, M59L, M59Y, G60A, N61D, N61E, N61K, N61R, Y62A, Y62F, Y62I, Y62M, Y62V, Y62W, A63D, A63E, A63Q, D64E, D64H, K65E, K65R, I66L, I66V, A67E, K68D, K68E, C69H, C69I, C69L, C69M, C69N, V70I, V70L, V70M, V70R, N71D, N71E, N71F, N71H, N71Y, K72A, K72R, S73A, S73T, K74D, K74E, K74R, S75G, S75P, S75Y, V76F, V76I, V76L, V76M, V76Y, W77F, W77Y, W78E, W78F, W78H, W78N, W78R, W78Y, R79D, R79S, R79W, R79Y, M80F, M80I, M80L, M80V, K81A, K81E, K81R, D82E, D82G, D82P, K83E, K83Q, K84Q, K84R, S85A, S85T, R86E, R86K, R86V, G87T, I88L, I88M, I88V, I89L, I89P, I89V, K90D, K90E, K90H, K90P, K90R, K90S, R91D, R91E, R91Q, I92D, I92M, P93D, P93E, E94A, E94D, E94G, E94N, L95K, L95M, L95N, L95R, G96H, G96K, G96N, G96R, L97I, L97M, L97V, V98I, V98M, V98T, V98Y, E99K, E99Q, E99R, E99T, V100Y, V100L, V100M, V100I, A101K, A101Y, A101R, A101H, K102E, K102Q, K102R, K102V, I104V, I104Y, I104F, I104L, I104M, V106I, I107C, I107V, G108A, G108C, C109I, C109A, C109M, V110M, V110L, V110I, A111T, A111V, A111Y, A111I, A111S, P112E, T113F, T113S, T113W, T113Y, T114N, N115W, N115R, N115H, N115Y, V117N, V117L, V117T, V117I, I118N, I118A, I118L, I118M, I118S, I118F, I118V, N119Q, N119I, N119L, N119M, N119T, N119S, V120T, V120A, V120P, V120M, M121V, M121A, M121L, M121C, M121I, Q122C, Q122F, Q122R, Q122Y, Q122H, Q122N, Q122S, Q122G, Q122V, Q122W, N123K, N123P, A124V, A124L, A124M, A124I, M125I, M125L, M125A, M125V, C126A, C126P, A127M, L128I, K129A, K129M, C130T, C130S, C130A, G131R, N132C, S133T, S133C, M134I, M134C, M134V, I135V, I135M, V136L, V136M, V136F, V136I, S137R, S137K, P138G, H139S, H139G, P140E, R141Q, R141K, R141Y, R141E, A142T, A142S, K143P, H144L, H144N, H144R, S145T, S145C, S145V, S146T, S146C, S146A, S146N, V147I, V147M, V147A, K148Y, K148L, K148A, K148E, K148H, K148R, T149V, T149I, T149L, T149M, T149F, V150I, V150M, V150G, V150A, E151D, E151K, E151R, E151Q, V152L, V152M, V152I, I153F, I153V, I153C, I153M, N154H, N154A, N154Q, N154K, N154R, E155R, E155K, E155D, E155Q, A156I, L157A, L157I, L157F, A158E, A158V, A158K, A159K, A159Q, A159D, A159E, L160V, L160C, L160A, M162I, M162A, M162F, M162Y, M162L, M162V, K164D, K164A, K164P, N165H, N165G, N165D, L166C, L166A, L166I, L166V, I167V, I167F, I167L, Q168T, Q168N, V169L, V169W, V169C, V169M, V169I, I170L, I170V, T171E, T171P, T171N, T171D, E172D, E172G, E172H, P173G, P173D, S174G, S174D, S174T, M175I, M175H, M175R, E176Q, E176D, L177V, L177T, L177A, L177I, S178V, S178A, S178T, S178G, A179D, A179N, A179E, G180H, G180Q, G180A, G180E, L181I, L181F, M182L, M182H, M182I, M182V, S183Q, S183K, S183D, S183A, S183H, S183E, A184H, A184P, A184S, A184G, V185C, V185I, D186R, L187H, L187K, L187F, L187I, L187V, L187M, C188I, C188L, C188V, I189S, I189F, I189T, I189M, I189V, I189L, C190P, C190A, C190F, T191R, G193S, P194T, P194R, P194K, P194A, G195A, L196M, L196I, L196V, V197I, V197M, V197G, V197A, K198Q, K198E, K198R, A199S, A199V, A200C, A200I, A200V, Y201M, Y201Q, Y201A, S202N, S202K, S202A, S202R, S202T, S202E, S202Q, S203N, S203H, S203A, K205R, K205T, P206V, P206R, P206K, A207V, A207S, A207C, A207T, I208V, I208T, I208Y, I208M, I208L, G209L, G209E, G209M, V210H, V210L, G211L, Q212A, Q212P, Q212G, G213K, N214S, N214K, V215S, V215N, V215A, V215C, V215D, V215P, Q216H, Q216A, Q216P, V217F, V217A, V217I, V217C, V217M, L218I, L218V, L218F, L218Y, V219M, V219F, V219L, V219I, D220C, D220F, R221D, R221P, D222T, D222S, A223C, D224N, L225V, D226E, Q227K, Q227R, Q227M, Q227E, V228A, A229W, A229V, A230R, A230D, A230P, A230Q, A230K, A230E, M231Q, M231R, M231I, M231K, V2321, V232F, V232T, V232C, V232Q, V232M, V232N, I233V, I233L, I233M, K234W, K234Q, K234F, K234A, K234M, G235A, G235S, R236K, R236C, R236W, R236A, R236I, T237F, T237Y, T237C, T237M, F238R, F238M, F238Q, F238Y, F238W, D239R, D239H, D239Q, N240Q, N240A, N240T, N240C, N240S, G241N, L243V, L243M, L243T, L243C, T245M, T245I, T245N, T245S, T245V, T245A, T245C, C246S, C246A, E247G, E247P, E247I, E247A, Q248D, Q248T, Q248E, Q248K, Q248S, Q248N, N249Y, N249R, V250I, V250L, V250M, I251Y, I251F, I251L, 1251V, C252V, C252I, P253D, P253Q, P253H, P253V, P253E, E254Q, E254K, E254R, E254D, D255E, K256V, K256M, K256I, K256R, K257A, K257F, K257H, K257Y, E258D, E259A, E259R, E259K, E259D, E259Q, M260I, M260V, M260F, M260L, I261L, I261M, I261V, A262Q, A262D, A262E, A262K, A263E, A263K, A263R, L264M, L264V, L264F, K265Q, K265R, K265A, K265E, K266A, K266E, H267Y, H267R, H267N, H267A, H267Q, G268A, A269S, A269G, A269C, Y270L, Y270F, Y270I, Y270M, Y270W, Y271F, Y271K, Y271V, Y271W, Y271R, Y271H, I272V, I272M, I272L, G273C, N274E, N274D, S275P, S275G, S275A, S275E, E276Q, E276M, E276R, E276D, D277Q, D277E, A278P, A278E, A279E, A279D, A279S, K280Q, K280R, K280T, L281V, L281M, L281I, R282Q, R282K, R282M, R282G, R282E, D283E, D283H, D283N, T284V, T284P, A285L, A285V, A285I, A285M, F286L, F286I, F286W, F286Y, F286V, N288R, N288D, N288E, G289M, G290H, P291K, P291L, P291E, P291T, P291H, P291A, P291N, V292I, V292M, S293A, S293V, S293I, S293N, K294S, K294N, K294P, K294E, K294R, E295R, E295K, E295A, E295Q, E295D, Y296W, Y296R, Y296V, Y296F, Y296H, Y296L, P297A, P297T, P297Q, P297V, P297L, P297H, P297I, G298A, A299K, A299Q, A299R, S300A, S300R, V301I, V301A, K302A, K302R, K302D, K302Q, K302E, K303Q, K303W, K303R, K303E, I304L, I304G, I304Y, A305I, A305V, Q306H, Q306K, Q306E, Q306R, L307I, L307V, L307M, S308A, S308T, I310G, I310V, I310L, I310F, Q311R, Q311T, Q311K, Q311S, Q311D, Q311E, G312A, G312D, G312T, G312K, I313L, I313V, P314A, E315D, D316E, D316G, D316N, A317G, A317T, K318R, K318T, K318Y, V319F, V319L, V319M, V319I, V319Y, I320Y, I320L, V321L, V321P, V321C, V321I, S322A, S322T, S322G, S322V, C323E, C323L, C323I, C323G, C323P, C323V, C323K, T324I, T324V, T324L, R325K, R325E, R325D, R325T, G326D, Y327W, Y327F, Y327V, G328D, G328T, G328S, K329E, K329R, K329P, K329D, K329N, D330E, E331S, E331D, E331H, E331M, E331Y, P332R, P332T, L333M, L333V, L333F, L333I, A334V, A334S, A334M, A334C, K335R, K335Q, K335H, K337Q, K337E, L338F, L338V, L338I, L338M, F339C, F339M, V341I, L342M, L342V, L342I, A343C, A343T, A343V, A343S, A343P, F344I, F344V, F344L, F344M, F345Y, F345V, F345M, F345L, F345I, T346R, T346K, T346P, Y347F, Y347V, D348E, D348K, D348S, K349S, K349G, K349D, K349N, K349T, W350V, W350E, W350I, W350L, W350F, E351D, D352Q, D352H, D352E, I354L, I354V, I354C, H355R, H355A, H355D, H355Q, H355S, H355E, I356M, I356L, I356V, A357C, K358N, K358R, T359A, T359D, T359S, T359N, T359E, N360S, N360T, L361D, L361V, E362L, E362T, E362N, E362S, M363Y, M363E, E364Q, E364Y, E364F, E364H, I366Y, I366K, I366F, I366A, I366L, H368L, H368A, S369T, S369A, V370C, V370A, V371M, V371I, V371A, V371W, V371S, V371Y, I372V, I372M, H373W, H373Y, H373F, S374T, N375H, N375Q, N375K, N375T, N375R, T376N, T376S, T376D, P377D, P377I, P377V, P377E, E378K, E378D, E378A, H379N, H379K, H379V, H379R, I380A, I380M, I380V, E381M, E381W, E381K, E381R, E381T, E381H, A382K, A382R, A382E, A382Q, V383M, V383L, V383Y, V383F, V383I, A384S, A384G, E385Q, E385L, E385R, E385K, A386E, A386Q, A386K, A386R, I387M, I387V, I387L, P388R, P388H, P388D, P388Q, P388E, V389A, V389C, V389T, S390T, S390N, S390C, S390G, R391M, R391T, R391I, F392V, F392I, F392M, A393M, A393I, A393W, A393L, A393G, V394I, V394K, Q396R, Q396E, Q396S, Q396D, V397I, V397W, V397A, V397P, V397F, V397Y, G398S, G398C, G398P, G398T, G399A, G399S, T400I, T400S, T400F, T400V, T400H, N401G, N401H, N401D, N401S, N401A, L402N, L402A, L402F, L402M, G403S, A405I, A405F, A405V, A405G, M406Y, M406K, M406F, M406L, M406W, D407H, D407Q, D407Y, D407E, N408T, N408S, N408H, L410F, L4101, L410M, N411T, N411G, N411P, N411A, P412F, P412R, T413A, T414M, T414F, T414V, T415P, T415S, L416M, L416F, L416I, C418F, C418I, C418T, T420S, T420R, W421Y, W421F, N423K, N423R, N423G, N424S, N424R, S425I, S425G, S425W, S425A, I426V, I426T, I426L, S427G, S427T, S427A, E428D, E428H, N429E, F4301, F430Y, F430L, F430V, T431G, T431S, T431N, Y432A, Y432P, Y432F, Y432W, Y433T, Y433R, Y433K, Y433H, Y433M, Y433E, H434R, H434Q, H434D, H434N, L435V, L435F, L435W, L435Y, L435M, M436T, M436L, M436F, M436I, M436V, N437H, N437Q, N437Y, L438V, L438T, L438W, L438C, L438Y, L438I, T439Q, T439S, T439R, T439K, R440K, R440W, V441L, V441I, V441M, S442G, S442A, Y443H, Y443W, Y443R, Y443F, R444E, R444P, R444K, V445I, V445Y, V445F, V445M, V445L, P446E, P446K, D447E, D447N, M448W, M448K, M448P, Y449E, Y449R, Y449F, Y449H, I450M, I450C, I450F, I450V, I450L, P451W, T452S, T452D, T452K, D453E, D453P, D453H, E454A, E454D, E455K, E455Q, E455D, I456F, I456L, I456V, I456W, I456M, W457M, W457Y, W457F, A458H, A458G, E459D, and any combination thereof.

55. The engineered ALDH of any one of claims 38 to 48, wherein the engineered ALDH comprises one or more amino acid alterations selected from the group consisting of: E2Q, E2T, E2P, Q3S, Q3K, A4G, A4S, V5I, V5T, K6F, D7E, D7Q, L9V, K11E, K11D, K11Q, K11R, M12L, M12A, A15R, A15K, S16A, R17K, I18K, 118E, I18Q, Q20F, Q20H, Q21A, Q21R, E22K, E22Q, E22A, F23Y, A24E, A24K, T25A, T25E, Y26L, Y26M, Y26W, P27D, P27T, Q28W, T30D, T30K, T30Q, V31I, V31L, D32N, D32E, K33E, K33Q, K33A, K33R, A34V, V35C, V35M, R36K, T37K, T37A, T37R, V38L, V38I, G39A, K40W, A41L, A41V, I42L, I42V, I42M, I42C, Y43E, Y43L, D44K, D44Q, N45H, N45R, E47D, L48E, L48M, L48D, L49I, H51N, H51E, H51Q, M52L, V54L, D55E, D55R, K58R, N61D, N61E, N61R, Y62F, Y62V, Y62W, A63D, A63E, A63Q, D64H, K65R, I66V, A67E, V70M, V70R, N71D, N71E, N71Y, N71F, N71H, S73T, K74E, K74R, S75G, W77Y, W78E, W78F, W78Y, W78H, W78N, R79D, R79W, M80F, M80V, M80I, K81A, K81R, D82G, K83E, K84R, K84Q, R86V, R86K, I88M, I89V, I89L, K90S, K90D, K90H, K90E, K90R, R91E, R91Q, I92D, P93D, E94G, E94D, L95K, L95M, L95R, G96N, L97I, L97V, L97M, V98Y, V98I, V98M, V98T, E99R, E99T, E99Q, E99K, V100Y, V100I, V100M, V100L, A101Y, A101H, K102R, K102Q, I104F, I104M, I107C, I107V, A111T, A111S, V1171, V117T, V117L, N119T, N119S, V120A, V120T, V120M, M121L, M121V, M121I, Q122H, A124I, A124V, A124L, A124M, M125I, M125L, M125V, C126A, L128I, K129A, K129M, C130A, C130S, S133C, S133T, V136L, V136I, H144R, S145C, S145T, S145V, S146N, V147A, V147I, V147M, K148E, K148L, K148A, K148R, T149M, T149V, T149L, T149I, V150A, V150I, V150M, E151K, E151D, E151Q, V152M, V152I, I153M, I153C, I153F, I153V, N154Q, N154K, E155K, E155R, E155D, E155Q, A156I, L157A, L157F, L157I, A158E, A158V, A158K, A159D, A159K, L160V, L160A, M162F, M162Y, M162V, M162A, M162L, K164A, K164D, K164P, N165D, N165H, L166V, L166C, L166I, I167L, I167V, I167F, Q168T, Q168N, V169C, V169L, V169M, V169I, I170V, T171D, T171E, T171P, T171N, E172D, E172G, E172H, S174T, M175I, M175H, E176D, L177A, L177T, L177I, G180E, L181I, S183Q, S183A, S183H, S183K, A184G, A184S, L187M, L187V, L187I, L187F, L187K, I189V, I189L, C190A, P194A, P194T, P194R, P194K, L196V, V197I, K198R, A199V, A200C, Y201A, S202A, S202T, S202R, S203N, S203H, S203A, P206V, A207V, A207T, A207C, I208V, I208M, I208Y, Q212A, Q212P, N214S, V215P, V215N, V215C, V215A, V215S, Q216A, Q216P, V217M, V217A, V217C, L218I, L218V, V219I, V219M, V219L, R221P, R221D, D222T, D222S, A223C, D224N, L225V, Q227M, Q227E, Q227K, V228A, A229V, A230D, A230E, A230P, A230K, M231R, M231Q, M231I, M231K, V232M, I233L, K234F, K234A, K234W, K234M, K234Q, T237F, T237Y, T237C, T237M, F238R, F238Q, F238M, N240T, L243V, T245I, C246S, C246A, Q248N, N249Y, V250M, I251F, I251L, C252V, C252I, P253H, E254K, E254R, E254D, D255E, K256I, K256M, K256V, K256R, K257Y, K257A, E258D, E259R, E259A, E259Q, E259D, M260V, M260L, 1261V, I261L, I261M, A262K, A263K, A263R, L264V, L264F, K265R, K265Q, K265E, K266E, H267R, Y270F, Y270L, Y270W, Y270M, Y270I, Y271W, Y271F, Y271K, Y271H, I272V, I272L, I272M, G273C, N274D, N274E, S275A, S275P, S275G, S275E, E276R, E276D, D277E, D277Q, A278E, A279S, A279E, K280T, L281I, L281V, L281M, R282K, R282Q, R282G, R282M, D283E, D283N, D283H, T284V, A285V, F286Y, F286V, F286I, N288E, G290H, P291L, P291A, P291T, P291H, P291N, P291K, S293A, S293I, S293N, K294R, E295D, E295R, E295Q, Y296L, Y296W, A299K, A299Q, A299R, S300A, V301A, K302A, K302E, K302Q, K303E, K303R, I304L, A305V, Q306E, Q306H, Q306K, Q306R, L307I, L307M, L307V, S308T, I310F, I310V, I310L, Q311D, Q311S, Q311R, Q311E, G312A, G312T, G312D, G312K, 1313V, I313L, E315D, D316G, D316E, D316N, A317G, A317T, K318R, V319L, V319M, I320Y, I320L, V321I, V321L, V321C, S322G, S322V, S322A, S322T, C323I, C323V, C323K, C323P, T324I, T324V, T324L, R325E, R325T, G326D, Y327F, Y327W, Y327V, G328S, K329P, K329R, K329D, K329N, D330E, E331D, P332T, P332R, L333I, L333M, L333F, A334S, A334V, A334C, A334M, K335R, F339M, V341I, L342I, L342M, A343S, A343C, A343V, F344M, F344I, F344V, F344L, F345L, F345M, F345Y, F345V, T346P, Y347F, Y347V, D348E, D348K, D348S, K349G, K349T, K349N, K349S, K349D, W350E, W350I, W350V, W350F, W350L, E351D, I354L, I354C, H355D, H355A, H355Q, H355S, H355E, I356L, I356V, K358R, K358N, T359S, T359A, T359N, T359E, N360S, E362S, E362N, E362T, M363E, I366A, I366K, I366Y, I366L, V370C, V370A, V371S, V371A, I372V, I372M, H373F, H373Y, H373W, S374T, N375H, N375T, N375K, N375R, N375Q, T376D, P377E, P377I, P377V, E378K, E378A, E378D, H379R, H379N, H379V, I380V, E381W, E381H, E381M, E381R, E381K, E381T, A382K, V383I, V383M, A384G, A384S, E385R, E385K, A386Q, A386K, A386R, I387M, I387V, I387L, P388R, P388E, P388Q, P388H, P388D, V389T, V389A, V389C, S390G, S390T, S390C, F392M, F392V, F392I, A393G, V394I, Q396E, Q396D, Q396S, V397W, V397I, V397Y, T400S, T400I, T400H, N401D, N401G, N401S, N401H, N401A, L402M, A405V, A405G, M406W, M406F, M406L, D407H, D407Q, L410M, N411G, N411A, N411P, N411T, T414V, T420S, W421F, N423G, N423R, S425I, S425A, S425G, I426V, S427A, S427T, E428D, T431N, Y433T, Y433H, Y433K, Y433M, Y433R, L435M, L435V, M436V, M436F, M436I, N437Q, L438I, L438T, L438Y, L438C, T439S, R440W, R440K, V441M, V441I, V441L, Y443W, Y443F, Y443R, R444K, P446K, D447E, D447N, M448W, M448K, M448P, Y449R, Y449H, I450V, T452S, T452K, D453H, D453P, E454A, E454D, E455K, E455D, W457F, A458H, A458G, E459D, and any combination thereof.

56. The engineered ALDH of any one of claims 38 to 48, wherein the engineered ALDH comprises one or more amino acid alterations selected from the group consisting of: I42V, N119T or N119S, T149I, A158K, S174T, C246A, E259D, A263R, A299R, Y327V, D348S, V370A, F392I, N401A, S427T, and any combination thereof.

57. The engineered ALDH of any one of claims 38 to 56, wherein the one or more amino acid alterations of the ALDH is an alteration described in TABLE 12.

58. The engineered ALDH of any one of claims 38 to 57, wherein the amino acid sequence, other than the one or more amino acid alterations, has at least 65%, at least 70%, at least 75%, at least 80%, at least 85%, at least 90%, at least 95%, at least 98%, at least 99%, or 100% sequence identity to the amino acid sequence referenced in SEQ ID NO: 3.

59. A recombinant nucleic acid encoding the engineered ALDH of any one of claims 38 to 58.

60. The recombinant nucleic acid of claim 59, wherein the nucleic acid comprises a nucleotide sequence encoding the engineered ALDH operatively linked to a promoter.

61. A vector comprising the recombinant nucleic acid of claims 59 or 60.

62. A recombinant nucleic acid encoding:
a) the engineered ACL of any one of claims 1 to 34; and
b) the engineered ADLH of any one of claims 38 to 58.

63. The recombinant nucleic acid of claim 62, wherein the nucleic acid comprises a nucleotide sequence encoding the engineered ACL operatively linked to a promoter.

64. The recombinant nucleic acid of claim 63, wherein the nucleic acid comprises a nucleotide sequence encoding the engineered ACL operatively linked to a first promoter and the engineered ALDH operatively linked to a second promoter.

65. A vector comprising the recombinant nucleic acid of any one of claims 62 to 64.

66. A non-naturally occurring microbial organism comprising a recombinant nucleic acid encoding an engineered ACL selected from any one of claims 1 to 34.

67. The non-naturally occurring microbial organism of claim 66, wherein the non-naturally occurring microbial organism further comprises a pathway that produces HHA-CoA and/or 1,6 hexanediol (1,6 HDO), or an ester or amide thereof.

68. The non-naturally occurring microbial organism of claim 67, wherein the non-naturally occurring microbial organism is capable of producing at least 10% more HHA-CoA and/or 1,6 HDO, or an ester or amide thereof compared to a control microbial organism that does not comprise any one of the nucleic acids of claims 35 or 36.

69. A non-naturally occurring microbial organism comprising a recombinant nucleic acid encoding an engineered ALDH selected from any one of claims 38 to 58.

70. The non-naturally occurring microbial organism of claim 69, wherein the non-naturally occurring microbial organism further comprises a pathway that produces HHAI and/or 1,6 HDO, or an ester or amide thereof.

71. The non-naturally occurring microbial organism of claim 70, wherein the non-naturally occurring microbial organism is capable of producing at least 10% more 6-HHAI and/or 1,6 HDO, or an ester or amide thereof compared to a control microbial organism that does not comprise any one of the nucleic acids of claims 59 or 60.

72. The non-naturally occurring microbial organism of any one of claims 66 to 68, or claims 69 to 71 wherein the microbial organism produces a decreased amount of one or more by-product as compared to a control microbial organism that does not comprise the recombinant nucleic acid of any one of claims 35 to 36 or any one of claims 59 to 60, respectively.

73. The non-naturally occurring microbial organism of claim 72, wherein the one or more by-product comprises adipic acid (ADA), 4-HB, 3HB, acetate, succinate, 3-oxoadipate, 3-hydroxyadipate, 2-hexenedioate or combinations thereof.

74. The non-naturally occurring microbial organism of any one of claims 66 to 73, wherein the microbial organism is capable of producing at least 10% less by-product compared to a control microbial organism that does not comprise the recombinant nucleic acid of any one of claims 35 to 36 or any one of claims 59 to 60.

75. A non-naturally occurring microbial organism comprising a recombinant nucleic acid encoding
a) an engineered ALDH selected from any one of claims 1 to 34; and
b) an engineered ACL selected from any one of claims 38 to 58;

76. The non-naturally occurring microbial organism of claim 72, wherein the non-naturally occurring microbial organism further comprises a pathway that produces 6-HHAI and/or 1,6 HDO, or an ester or amide thereof.

77. The non-naturally occurring microbial organism of claim 76, wherein the non-naturally occurring microbial organism is capable of producing at least 10% more HHA-CoA and/or 6-HHAI and/or 1,6 HDO, or an ester or amide thereof compared to a control microbial organism that does not comprise any one of the nucleic acids of claims 35, 36, 59, 60, or any combination thereof.

78. The non-naturally occurring microbial organism of any one of claims 72 to 77, wherein the microbial organism produces a decreased amount of one or more by-product as compared to a control microbial organism that does not comprise the recombinant nucleic acid of any one of claims 35, 36, 59,60, or any combination thereof.

79. The non-naturally occurring microbial organism of claim 78, wherein the one or more by-product is 4-HB, 3HB, ADA, or both.

80. The non-naturally occurring microbial organism of any one of claims 72 or 79, wherein the microbial organism is capable of producing at least 10% less by-product compared to a control microbial organism that does not comprise the recombinant nucleic acid of any one of claims 35, 36, 59, 60, or any combination thereof.

81. The non-naturally occurring microbial organism of any one of claims 66 to 80, wherein the one or more enzymes of the pathway are encoded by an exogenous nucleic acid.

82. The non-naturally occurring microbial organism of claim 81, wherein the exogenous nucleic acid is heterologous or homologous.

83. The non-naturally occurring microbial organism of any one of claims 66 to 82, wherein the non-naturally occurring microbial organism is in a substantially anaerobic culture medium.

84. The non-naturally occurring microbial organism of any one of claims 66 to 83, wherein the microbial organism is a species of bacteria, yeast, or fungus.

85. A method for producing HHA-CoA and/or 6-HHAI and/or 1,6 HDO, or an ester or amide thereof, comprising culturing the non-naturally occurring microbial organism of any one of claims 66 to 84 under conditions and for a sufficient period of time to produce the HHA-CoA and/or 6-HHAI and/or 1,6 HDO, or an ester or amide thereof.

86. The method of claim 85, wherein the method further comprises separating the HHA-CoA and/or 6-HHAI and/or 1,6 HDO, or an ester or amide thereof from other components in the culture.

87. The method of claim 86, wherein the separating comprises extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

88. A culture medium comprising the HHA-CoA and/or 6-HHAI and/or 1,6 HDO, or an ester or amide thereof produced by the method of any one of claims 85 to 87, wherein the HHA-CoA and/or 6-HHAI and/or 1,6 HDO, or an ester or amide thereof has a carbon-12, carbon-13 and carbon-14 isotope ratio that reflects an atmospheric carbon dioxide uptake source.

89. A HHA-CoA and/or 6-HHAI and/or 1,6 HDO, or an ester or amide thereof produced according to the method of any one of claims 85 to 87.

90. The HHA-CoA and/or 6-HHAI and/or 1,6 HDO, or an ester or amide thereof of claim 88 or 89, wherein the HHA-CoA and/or 6-HH and/or 1,6 HDO, or an ester or amide thereof has an Fm value of at least 80%, at least 85%, at least 90%, at least 95% or at least 98%.

91. A composition comprising the HHA-CoA and/or 6-HHAI and/or 1,6 HDO, or an ester or amide thereof of claim 89 or 90 and a compound other than the HHA-CoA and/or 6-HHAI and/or 1,6 HDO, or an ester or amide thereof.

92. The composition of claim 91, wherein the compound other than the HHA-CoA and/or 6-HHAI and/or 1,6 HDO, or an ester or amide thereof is a trace amount of a cellular portion of a non-naturally occurring microbial organism having a pathway that produces HHA-CoA and/or 6-HHAI and/or 1,6 HDO, or an ester or amide thereof.

93. A composition comprising the HHA-CoA and/or 6-HHAI and/or 1,6 HDO, or an ester or amide thereof of claim 89 or 90 or a cell lysate or culture supernatant thereof.

94. Use of the engineered ACL of any one of claims 1 to 34 as a biocatalyst.

95. A composition comprising the engineered ACL of any one of claims 1 to 34 and at least one substrate for the engineered ACL.

96. The composition of claim 95, wherein the engineered ACL can react with the substrate under in vitro conditions.

97. The composition of claim 95 or 96, wherein the substrate is 6-HHA.

98. Use of the engineered ALDH of any one of claims 38 to 58 as a biocatalyst.

99. A composition comprising the engineered ALDH of any one of claims 38 to 58 and at least one substrate for the engineered ALDH.

100. The composition of claim 99, wherein the engineered ALDH can react with the substrate under in vitro conditions.

101. The composition of claim 99 or 100, wherein the substrate is HHA-CoA.

102. The composition of claim 99 or 100, wherein the substrate is adipyl-CoA.


SEQUENCE LISTING (104 sequences)

SEQ ID NO: 1 - protein, 597 aa, unidentified
MTQIIHNATIPNIPVDQLYDGKITDFIRSGGHSNETKPSVIDAKTGQTLSQAEMWQLSDKYAALLSSQYGLCRHRDNELDPSMGDVLITFFGNVILAPVVHWAALDLGATISPGSTGYSAQDLAHQFRVTTPKVVVYAKAFKDVVDEATKLYNSPNPPALVELEALDKQARMVGNHKVEHTRKIKLAPHESRTRIAYLGMSSGTSGGVSKAVRLTHSNLTSCSEISNKASESLATDQQIAAAIIPVSHLFGLSKFLIGNPHAGATTVYHNGFDLIEVLEAQKKYKVNSWTLVPPIIVLLTKHPIVEKYIPSLRAHMRAILSGAAPLGANVTEALLTRVTGNKFGESPEGGLRIVQGYGLTETSPVATLFDPEDKERHIRSCGKLVPNSQVRIVNEDGVDQPAYDVDPNELDEAIKQGTLPVGELWIRGPQVMDGYHNNPEANEACFVKADDAEADTAYYNRHWFRTGDVALVDKQGRYMIVDRTKEMIKSQGKQVAPAELEDMLLGHAQVADTAVIGIQDVEKGNEAPRAFVVLKDPKYDAVEIKTWLDKQLPKYKQLHAGIVVIDAIPKNASGKILRRLLRARKDDVVLGLNKAKL

SEQ ID NO: 2 - protein, 603 aa, unidentified
MNQIDASGIELLPHDVHQESRPDGSILLRSTHSLGAVAPNTGAWLHRWAKEAPGRVAIAERAGAGWREHTYNELLNMVRNVAGALLARGLGADSPIVVMSGNGVDHAILSLAAQYVGVPIVPLAEQYSLIPEAHGRLIYVLEKVKPAMAFVSDAAQYASALSLPQLEGVELVAVKTDGAPVPVTAFADLLGHGQGGVDDAHAKVGPDTLAKILFTSGSSSDPKGVLTTHRMMCANQTQISDALRFLKARPPRILDWLPWNHVFGGSHNFNMMLANGGSLYIDNGKPTKAGFEDTLRNIKEHPGTLAFNVPVGYAMLVNAMERDPALRRAYFKELDLIFYAGASLPQDVWESLERFAMEERGSLPLMASSWGMTETAPATIMVHEPIGRSGVIGVPLPGAEVKLLPDDEMRCELRVKGPNIMRGYFNDPAKTAEAFDDEGFLVTGDAVRFVDDADPSRGLVFDGRVSEDFKLLTGTWVQAGVLRLDALKALAGLVQDVVICGHDRDQIGLFIFPLPGAVSDTSAGAVIDPALSAQIEERLRDMAKTATGSAKRINRAIILSEPPQLKDHEITDKGSLNIRKILTRRADLLERLYDNEDPALIRV

SEQ ID NO: 3 - protein, 459 aa, Acidaminococcus sp.
MEQAVKDYLDKMVAASRIAQQEFATYPQETVDKAVRTVGKAIYDNAELLAHMAVDETKMGNYADKIAKCVNKSKSVWWRMKDKKSRGIIKRIPELGLVEVAKPIGVIGCVAPTTNPVINVMQNAMCALKCGNSMIVSPHPRAKHSSVKTVEVINEALAALGMPKNLIQVITEPSMELSAGLMSAVDLCICTGGPGLVKAAYSSGKPAIGVGQGNVQVLVDRDADLDQVAAMVIKGRTFDNGVLCTCEQNVICPEDKKEEMIAALKKHGAYYIGNSEDAAKLRDTAFPNGGPVSKEYPGASVKKIAQLSGIQGIPEDAKVIVSCTRGYGKDEPLAKEKLFPVLAFFTYDKWEDAIHIAKTNLEMEGIGHSVVIHSNTPEHIEAVAEAIPVSRFAVNQVGGTNLGGAMDNGLNPTTTLGCGTWGNNSISENFTYYHLMNLTRVSYRVPDMYIPTDEEIWAE

SEQ ID NO: 4 - protein, 468 aa, Clostridium sp.
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 5 - protein, 475 aa, unidentified
MQINDIESAVRKILAEELDNASSNSAAVTNDDGHRGIFTNVDDAIAAAKAAQEVYRDKPIAVRQQVIDAIKEGFRPYIEKMAKDIKEETGMGTVEAKIAKLHNALYNTPGPEILEPVVENGDGGMVMYERLPYGVIGAVGPSTNPSETVIANAIMMLAGGNTLYFGAHPGAKNITRWTIEKMNDFIADATGLHNLVVSIETPTIESVQQMMQHPDIAMLAVTGGPAVVHQAMTSGKKAVGAGPGNPPAMVDATADIDLAAHNIIASASFDNDILCTAEKEVVAESSIKDELIRKMQDEGAFVVNREQADKLADMCIQDNGTPDRKFVGKDATYILDQANIPYTGHPVEIICELPKEHPLVMTEMLMPILPVVSCPTFDDVLKTAVEVEQGNHHTATIHSNNLKHINNAAHRMQCSIFVVNGPSYVGTGVADNGAHSGASALTIATPTGEGTCTARTFTRRVRLNSPQGFSVRNWY

SEQ ID NO: 6 - protein, 462 aa, unidentified
MTVNEQLVQDIIKNVVASMQLTQTNKTELGVFDDMNQAIEAAKEAQLVVKKMSMDQREKIISAIRKKTIEHAETLARMAVEETGMGNVGHKILKHQLVAEKTPGTEDITTTAWSGDRGLTLVEMGPFGVIGAITPCTNPSETIICNTIGMLAGGNTVVFNPHPAAIKTSNFAVQLINEASLSAGGPVNIACSVRKPTLDSSKIMMSHQDIPLIAATGGPGVVTAVLQSGKRGIGAGAGNPPVLVDETADIRKAAEDIINGCTFDNNLPCIAEKEVVAIDAIANELMNYMVKEQGCYAITKEQQEKLTNLVITPKGLNRNCVGKDARTLLGMIGIDVPSNIRCIIFEGEKEHPLISEELMMPILGIVRAKSFDDAVEKAVWLEHGNRHSAHIHSKNVDRITTYAKAIDTAILVKNAPSYAAIGFGGEGFCTFTIASRTGEGLTSASTFTKRRRCVMSDSLCIR

SEQ ID NO: 7 - protein, 464 aa, Salmonella sp.
MNTSELETLIRTILSEQLTTPAQTPVQPQGKGIFQSVSEAIDAAHQAFLRYQQCPLKTRSAIISAMRQELTPLLAPLAEESANETGMGNKEDKFLKNKAALDNTPGVEDLTTTALTGDGGMVLFEYSPFGVIGSVAPSTNPTETIINNSISMLAAGNSIYFSPHPGAKKVSLKLISLIEEIAFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAVCLPEGQANKKLVGKSPSAMLEAAGIAVPAKAPRLLIALVNADDPWVTSEQLMPMLPVVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 8 - DNA, 1794 bp, unidentified
  source 1..1794: mol_type = unassigned DNA
atgacacagattatccataacgccaccatccccaatatccccgtggaccagctctacgacggcaagatcaccgacttcattcgaagcggaggccactccaacgaaaccaagccttcggtgatcgatgccaaaacaggccagactctttcccaggcggaaatgtggcagctgtcggacaagtacgcggcacttctaagctcgcagtacggtctatgcagacacagagacaacgagctggacccatccatgggagatgtgctcatcaccttttttggaaacgtgatcctcgcccctgtggtccattgggctgctctcgacctcggagcaaccatttctcccggatccacaggctactcggcccaggatctcgctcaccagttcagagtcaccactcccaaggtcgttgtgtatgccaaggcgttcaaggatgtggtggacgaggctacgaagctgtacaactcgcccaacccccccgcacttgttgagctcgaggcgctggacaaacaggcccgaatggttggaaaccacaaggtcgaacacacccgaaagatcaagctggccccccacgagtcgcgaactcgaatcgcgtaccttggcatgtcttcaggtacctccggaggagtttcaaaggctgtcaggctcacccattccaatctcacgtcgtgttctgaaatctctaacaaagcctccgagtccctcgcaactgaccagcagatcgctgccgccattatccccgtgtctcatctgtttggactgtcgaaattcctcattggcaacccccacgccggagccaccactgtctatcacaatgggttcgatctgatcgaggtgctggaggcacagaagaagtataaagtcaactcgtggaccctggtaccccccatcattgtactgctcaccaaacaccccattgtcgagaagtacattccttctctcagggcccacatgcgagccatcctctcgggagctgctcctctgggtgccaatgtcacagaggctcttttgaccagagtcactggcaacaagtttggcgagtcccccgagggcggtctgcgaatcgtccagggctacggacttacagagacgtctcccgtagccactctgtttgaccccgaagacaaggaacgacacattaggtcgtgtggaaagctcgtgcccaactcgcaggttcgaattgtgaacgaagacggcgttgatcagcccgcctacgatgtggaccccaacgagctggacgaggccatcaaacagggcactctgccagtcggagagctttggatcagaggtccccaggttatggacggctaccataacaaccccgaggccaacgaagcctgtttcgtcaaggctgacgatgctgaagcagatactgcctactataacagacactggttccgaaccggagacgttgctctggtcgacaagcagggcagatacatgattgtggaccgaaccaaggagatgatcaagagtcagggtaagcaggttgctcctgccgagctcgaagacatgctcctgggacacgcacaggtggcagataccgcagtcattggtattcaggacgtggagaagggtaacgaggctccccgagcttttgttgtgctcaaggacccgaagtacgacgctgtggagattaaaacatggctggacaagcagcttcccaagtacaagcagcttcatgctggcatcgtggtgattgatgccattcccaagaacgccagtggcaagattctgcgacgattgttgcgtgctagaaaggacgatgttgttctgggtctcaacaaggccaagctgtaa

SEQ ID NO: 9 - DNA, 1812 bp, unidentified
  source 1..1812: mol_type = unassigned DNA
atgaaccagatagatgccagcggcatagagctgctgccacatgacgtgcatcaggaaagccgcccggatgggtcaattctgcttcggtctacgcattctcttggggcggtcgcgccgaacactggcgcatggttgcaccggtgggcgaaagaggcgccggggcgcgttgccatcgcagaacgtgcgggtgcgggctggcgggaacatacctataacgaattgctaaacatggtgcgcaacgttgctggcgcgcttttggcgcggggcttgggcgcggatagcccaatcgtggtcatgtccggcaacggggttgatcatgcgatcctatcgcttgccgcgcaatatgttggtgtacccatcgtcccccttgcagagcagtattcgctgattcccgaagcccacgggcggctgatctatgtgcttgaaaaggtgaagcccgcgatggcgtttgtttcggacgcggcgcagtatgcctctgcgctgtcactgccacagcttgaaggggtggaactggtcgcggtcaaaacagatggcgcgcctgtgccagtgaccgcatttgccgatttgctgggccacggacaggggggtgtcgacgacgcccacgccaaagtcgggccagatacgctggctaagattttgttcacttcggggtcgtcctcggacccaaagggggttctgacgacgcaccgcatgatgtgtgccaaccaaacgcagatctctgacgcgctgcggttcttgaaggcgcggccaccccgtattctggactggctgccatggaaccatgtgttcggcggaagccacaacttcaacatgatgctcgccaatggcggcagcctttatatcgacaacggaaagcccaccaaagctggcttcgaagacacccttcgcaacatcaaagagcatccgggaaccttggcgttcaacgtgcccgtgggctatgcgatgcttgtcaacgcgatggagcgtgacccggccctgcgacgcgcctatttcaaagaacttgaccttatcttttacgcgggcgcgtccctgccgcaggatgtgtgggaaagtctggaacgtttcgcgatggaagaacggggcagcctgcctctgatggcgtccagctggggcatgaccgaaaccgcgcccgccacgattatggtccatgaacctatcgggcggtcaggtgtgatcggtgttccgttgccgggggcagaggtcaagctgttgcccgatgatgaaatgcgctgtgaattgcgggtcaaagggccgaacatcatgcgcggctatttcaacgatccggcgaaaaccgccgaagcgtttgatgacgaggggttcctggtaaccggcgatgccgtgcgctttgtcgacgatgcggatccatcgcgcgggttggtgtttgacgggcgtgtctctgaagatttcaagttgttgaccggaacatgggtgcaggcaggcgtcttgcgtctggatgcgcttaaggcgcttgccggtttggtgcaggacgttgtgatctgcggccatgatcgtgatcagattggcctgttcatctttccgcttccgggggcggtgtctgacacctctgccggggcggtcattgatccggcgcttagcgcccagatcgaagagcgcctgcgcgacatggccaaaaccgcaaccgggtctgccaagcgcattaaccgcgcaattatcctgtcagagccgccgcagttgaaggaccacgagatcaccgacaagggcagcctgaacatccgcaaaatcctgacacgccgcgccgatctgttggaacggctttatgacaacgaagacccggccctgatccgggtttga

SEQ ID NO: 10 - DNA, 1380 bp, synthetic construct
  source 1..1380: mol_type = other DNA
atggaacaggccgttaaagattatctggataaaatggttgcagcaagccgtattgcacagcaagaatttgcaacctatccgcaagaaaccgttgataaagcagttcgtaccgtgggtaaagccatttatgataatgcagaactgctggcacatatggcagttgatgaaaccaaaatgggtaactatgccgacaaaattgccaaatgcgtgaacaaaagcaaaagcgtttggtggcgtatgaaagacaaaaaaagccgtggtattatcaaacgcattccggaactgggtttagttgaagttgcaaaaccgattggtgttattggttgtgttgcaccgaccaccaatccggttattaatgttatgcagaatgcaatgtgtgccctgaaatgtggtaatagcatgattgttagtccgcatccgcgtgcaaaacattcaagcgttaaaaccgtggaagtgattaatgaagcactggcagcactgggcatgccgaaaaatctgattcaggttattaccgaaccgagcatggaactgagcgcaggtctgatgagcgcagttgatctgtgtatttgtaccggtggtcctggtctggttaaagcagcatatagcagcggtaaaccggcaattggtgttggtcagggtaatgttcaggttctggttgatcgtgatgccgatctggatcaggttgcagccatggttattaaaggtcgcacatttgataatggtgttctgtgtacctgtgaacagaatgttatttgtcccgaggataaaaaagaagagatgattgcagccctgaaaaaacatggtgcctattatatcggtaatagcgaagatgcagcaaaactgcgtgataccgcatttccgaatggtggtccggttagcaaagaatatccgggtgccagcgtgaaaaaaatcgcacagctgagcggtattcagggtattccggaagatgcaaaagttattgttagctgtacccgtggttatggtaaagatgaaccgctggcaaaagaaaaactgtttccggttctggcctttttcacctatgataaatgggaagatgccatccatattgccaaaaccaatctggaaatggaaggtattggtcatagcgttgtgattcatagcaatacaccggaacatattgaagcagttgcagaagcaattccggttagtcgttttgcagttaatcaggttggtggcaccaatttaggtggtgcaatggataatggtctgaatccgaccacaaccttaggttgcggcacctggggtaataatagcattagcgaaaacttcacctactatcatctgatgaatctgacccgtgttagctatcgtgttccggatatgtatattccgaccgatgaagaaatttgggcagaataa

SEQ ID NO: 11 - DNA, 1407 bp, synthetic construct
  source 1..1407: mol_type = other DNA
atgaataaagacacactaatacctacaactaaagatttaaaagtaaaaacaaatggtgaaaacattaatttaaagaactacaaggataattcttcatgtttcggagtattcgaaaatgttgaaaatgctataagcagcgctgtacacgcacaaaagatattatcccttcattatacaaaagagcaaagagaaaaaatcataactgagataagaaaggccgcattacaaaataaagaggtcttggctacaatgattctagaagaaacacatatgggaagatatgaggataaaatattaaaacatgaattggtagctaaatatactcctggtacagaagatttaactactactgcttggtcaggtgataatggtcttacagttgtagaaatgtctccatatggtgttataggtgcaataactccttctacgaatccaactgaaactgtaatatgtaatagcataggcatgatagctgctggaaatgctgtagtatttaacggacacccatgcgctaaaaaatgtgttgcctttgctgttgaaatgataaataaggcaattatttcatgtggcggtcctgaaaatctagtaacaactataaaaaatccaactatggagtctctagatgcaattattaagcatccttcaataaaacttctttgcggaactgggggtccaggaatggtaaaaaccctcttaaattctggtaagaaagctataggtgctggtgctggaaatccaccagttattgtagatgatactgctgatatagaaaaggctggtaggagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattcttagatgaaatagatgttgagtctccttcaaatgttaaatgcataatctgcgaagtaaatgcaaatcatccatttgttatgacagaactcatgatgccaatattgccaattgtaagagttaaagatatagatgaagctattaaatatgcaaagatagcagaacaaaatagaaaacatagtgcctatatttattctaaaaatatagacaacctaaatagatttgaaagagaaatagatactactatttttgtaaagaatgctaaatcttttgctggtgttggttatgaagcagaaggatttacaactttcactattgctggatctactggtgagggaataacctctgcaaggaattttacaagacaaagaagatgtgtacttgccggctaa

SEQ ID NO: 12 - DNA, 1428 bp, synthetic construct
  source 1..1428: mol_type = other DNA
atgcagattaacgatattgaaagtgctgttcgcaagatcctagcagaagaattagataatgctagctctaacagtgcggcagttactaatgatgatggacatcgtggtatttttactaatgttgatgacgcaattgctgctgctaaagctgcccaagaagtatatcgtgacaagccaattgcagtgcggcaacaggtgattgatgctatcaaggaaggtttccgtccttacattgaaaagatggcaaaggacattaaagaagaaaccggaatgggaacagtcgaagcaaagattgctaagttgcacaatgctttgtacaacactcctggtccagaaattcttgaaccggttgtcgaaaatggtgatggcggaatggttatgtatgaacggttgccatacggtgttatcggtgctgttggtccaagtactaacccatccgaaacagttattgccaatgcaattatgatgcttgcaggtggaaataccctttactttggtgctcaccctggtgctaagaatatcactcgttggacgattgaaaagatgaatgatttcattgctgatgcaactggtcttcacaatttagttgtaagcattgaaacaccaacaattgaatctgttcaacaaatgatgcaacatcctgatattgctatgcttgccgtaaccggtggacctgctgttgttcaccaagcaatgactagcggtaagaaggctgttggtgctggtcctggtaatcctcctgcaatggttgatgctaccgctgatattgacttagctgctcataatattattgcttcagcttcattcgataatgacattttatgtactgctgaaaaggaagttgttgccgaaagcagtattaaggatgaattaatccgcaagatgcaagatgaaggtgcatttgttgttaaccgtgaacaggctgacaaactcgccgatatgtgcatccaagacaacggtactcctgatcggaagtttgttggaaaggatgcaacctacatcttagatcaagctaatattccatatactggtcacccggtagaaattatttgtgaattgccaaaggaacacccattggtaatgactgaaatgttgatgccaatcttaccagttgtttcttgcccaacatttgatgatgttttgaagactgctgttgaagtagaacaaggcaaccaccatactgcaactattcactccaacaacttaaagcatattaataatgctgctcaccggatgcaatgttcaatcttcgttgttaatggtccatcctatgttggaacaggtgttgctgataatggtgctcactcaggtgcttcagcattaacaattgctacgccaaccggtgaaggaacttgtactgcaagaacctttacgcgtcgtgttcgtttgaattcgccacaagggttctcagttcgtaattggtactaa

SEQ ID NO: 13 - DNA, 1389 bp, synthetic construct
  source 1..1389: mol_type = other DNA
gtgacagtgaatgaacaattggttcaagacatcattaaaaatgtcgtagcgagtatgcaattgacacaaacaaataagacagaacttggcgtattcgacgacatgaatcaggcaattgaagctgcaaaagaagctcaattggttgtaaagaagatgtcgatggatcagagagagaaaatcatctctgcgatacgtaagaaaacgattgaacatgcagaaacacttgcccgtatggcggtagaagaaactggcatgggtaatgttggacataagattttaaaacaccagctggtagcagaaaagacacctggaacagaagatatcacgactactgcttggtctggtgatcgtggtttaaccttagttgagatgggaccatttggtgtgattggtgcaataacaccatgtactaacccaagtgaaacaatcatttgtaacacgattggcatgttagctggtggaaacacagtggtatttaatccgcatccagcagcgataaagacctcgaactttgctgtacagttaattaatgaagcatcactttcggctggaggtccagtcaacattgcttgttctgtgagaaaacctaccctagatagcagtaagataatgatgtctcatcaggacattccattgattgctgctactggaggtccaggcgtagttactgctgtattacaaagcggaaaacgaggaatcggtgcaggagcaggaaatccaccggttttagtagatgaaacagctgatattagaaaagcagccgaagatatcattaatggatgtacctttgataataatcttccatgtattgcagaaaaagaagttgttgcaattgatgctatagcaaatgaattaatgaactacatggtaaaagaacaaggttgttatgcaatcaccaaagaacagcaagaaaagttaactaatcttgtgataacacccaaaggattaaatcgtaattgcgttggtaaagatgccagaacattacttggtatgattggaattgatgttccatccaatattcgttgtatcatttttgaaggtgagaaagaacatcctttgatttctgaagagttaatgatgccgattcttggaattgttcgagctaagagcttcgatgatgcggtagaaaaagcggtttggttagaacatggtaatcgacattccgcacatattcattccaagaatgtggaccgtatcacaacttatgcaaaagcgattgatacagctatccttgttaaaaatgcaccttcctatgcagcaatcggatttggtggtgaaggattttgtacctttactatagcaagtagaaccggtgaaggcttgacgagtgcaagtacatttacaaaacgcagacgatgcgtgatgtcggatagtttgtgtatccgataa

SEQ ID NO: 14 - DNA, 1395 bp, synthetic construct
  source 1..1395: mol_type = other DNA
atgaatacttctgaactcgaaaccctgattcgcaccattcttagcgagcaattaaccacgccggcgcaaacgccggtccagcctcagggcaaagggattttccagtccgtgagcgaggccatcgacgccgcgcaccaggcgttcttacgttatcagcagtgcccgctaaaaacccgcagcgccattatcagcgcgatgcgtcaggagctgacgccgctgctggcgcccctggcggaagagagcgccaatgaaacggggatgggcaacaaagaagataaatttctcaaaaacaaggctgcgctggacaacacgccgggcgtagaagatctcaccaccaccgcgctgaccggcgacggcggcatggtgctgtttgaatactcaccgtttggcgttatcggttcggtcgccccaagcaccaacccgacggaaaccatcatcaacaacagtatcagcatgctggcggcgggcaacagtatctactttagcccgcatccgggagcgaaaaaggtctctctgaagctgattagcctgattgaagagattgccttccgctgctgcggcatccgcaatctggtggtgaccgtggcggaacccaccttcgaagcgacccagcagatgatggcccacccgcgaatcgcagtactggccattaccggcggcccgggcattgtggcaatgggcatgaagagcggtaagaaggtgattggcgctggcgcgggtaacccgccctgcatcgttgatgaaacggcggacctggtgaaagcggcggaagatatcatcaacggcgcgtcattcgattacaacctgccctgcattgccgagaagagcctgatcgtagtggagagtgtcgccgaacgtctggtgcagcaaatgcaaaccttcggcgcgctgctgttaagccctgccgataccgacaaactccgcgccgtctgcctgcctgaaggccaggcgaataaaaaactggtcggcaagagcccatcggccatgctggaagccgccgggatcgctgtccctgcaaaagcgccgcgtctgctgattgcgctggttaacgctgacgatccgtgggtcaccagcgaacagttgatgccgatgctgccagtggtaaaagtcagcgatttcgatagcgcgctggcgctggccctgaaggttgaagaggggctgcatcataccgccattatgcactcgcagaacgtgtcacgcctgaacctcgcggcccgcacgctgcaaacctcgatattcgtcaaaaacggcccctcttatgccgggatcggcgtcggcggcgaaggctttaccaccttcactatcgccacaccaaccggtgaagggaccacgtcagcgcgtacttttgcccgttcccggcgctgcgtactgaccaacggcttttctattcgctaa

SEQ ID NO: 15 - protein, 8 aa, synthetic construct
RTYTQKEL

SEQ ID NO: 16 - protein, 8 aa, synthetic construct
RTYTQREL

SEQ ID NO: 17 - protein, 4 aa, synthetic construct
RTYT

SEQ ID NO: 18 - protein, 5 aa, synthetic construct
CIEAA

SEQ ID NO: 19 - protein, 5 aa, synthetic construct
TIEAA

SEQ ID NO: 20 - protein, 5 aa, synthetic construct
VTPAN

SEQ ID NO: 21 - protein, 5 aa, synthetic construct
MRHTG

SEQ ID NO: 22 - protein, 5 aa, synthetic construct
MRITG

SEQ ID NO: 23 - protein, 5 aa, synthetic construct
SIDAF

SEQ ID NO: 24 - protein, 5 aa, synthetic construct
RIESF

SEQ ID NO: 25 - protein, 5 aa, synthetic construct
HFDSF

SEQ ID NO: 26 - protein, 5 aa, synthetic construct
HFDAF

SEQ ID NO: 27 - protein, 5 aa, synthetic construct
HIDSF

SEQ ID NO: 28 - protein, 6 aa, synthetic construct
GKWSQD

SEQ ID NO: 29 - protein, 7 aa, synthetic construct
GKWSQSY

SEQ ID NO: 30 - protein, 5 aa, synthetic construct
GKWSQ

SEQ ID NO: 31 - protein, 8 aa, synthetic construct
GKWSQDLK

SEQ ID NO: 32 - protein, 8 aa, synthetic construct
GKWSQQLQ

SEQ ID NO: 33 - protein, 4 aa, synthetic construct
QDLQ

SEQ ID NO: 34 - protein, 5 aa, synthetic construct
DISAC

SEQ ID NO: 35 - protein, 6 aa, synthetic construct
ILLANV

SEQ ID NO: 36 - protein, 8 aa, synthetic construct
ILLANVHY

SEQ ID NO: 37 - protein, 8 aa, synthetic construct
ILLLNVHY

SEQ ID NO: 38 - protein, 6 aa, synthetic construct
ILLLNV

SEQ ID NO: 39 - protein, 8 aa, synthetic construct
ILLLNVHK

SEQ ID NO: 40 - protein, 8 aa, synthetic construct
ILLHNVHK

SEQ ID NO: 41 - protein, 8 aa, synthetic construct
ILLSNVHY

SEQ ID NO: 42 - protein, 4 aa, synthetic construct
IMPR

SEQ ID NO: 43 - protein, 4 aa, synthetic construct
IMNR

SEQ ID NO: 44 - protein, 9 aa, synthetic construct
PEEMLRAIQ

SEQ ID NO: 45 - protein, 9 aa, synthetic construct
PEEMLQAIQ

SEQ ID NO: 46 - protein, 8 aa, synthetic construct
EEMLQAIQ

SEQ ID NO: 47 - protein, 8 aa, synthetic construct
EEMLRAIQ

SEQ ID NO: 48 - protein, 8 aa, synthetic construct
KEMLQAIQ

SEQ ID NO: 49 - protein, 4 aa, synthetic construct
QAIQ

SEQ ID NO: 50 - protein, 4 aa, synthetic construct
RAIQ

SEQ ID NO: 51 - protein, 5 aa, synthetic construct
TVFFA

SEQ ID NO: 52 - protein, 5 aa, synthetic construct
TSFFA

SEQ ID NO: 53 - protein, 5 aa, synthetic construct
TFFFA

SEQ ID NO: 54 - protein, 5 aa, synthetic construct
TMFFA

SEQ ID NO: 55 - protein, 5 aa, synthetic construct
TVFHA

SEQ ID NO: 56 - protein, 6 aa, synthetic construct
SHVRSV

SEQ ID NO: 57 - protein, 6 aa, synthetic construct
SHVRTV

SEQ ID NO: 58 - protein, 4 aa, synthetic construct
RELE

SEQ ID NO: 59 - protein, 7 aa, synthetic construct
NTENAPR

SEQ ID NO: 60 - protein, 7 aa, synthetic construct
NTENAPD

SEQ ID NO: 61 - protein, 6 aa, synthetic construct
TENAPD

SEQ ID NO: 62 - protein, 6 aa, synthetic construct
TQNAPR

SEQ ID NO: 63 - protein, 6 aa, synthetic construct
TENAPR

SEQ ID NO: 64 - protein, 6 aa, synthetic construct
TQNAPN

SEQ ID NO: 65 - protein, 4 aa, synthetic construct
NNPN

SEQ ID NO: 66 - protein, 5 aa, synthetic construct
RASFA

SEQ ID NO: 67 - protein, 4 aa, synthetic construct
EDGH

SEQ ID NO: 68 - protein, 4 aa, synthetic construct
EDGY

SEQ ID NO: 69 - protein, 4 aa, synthetic construct
RHPD

SEQ ID NO: 70 - protein, 5 aa, synthetic construct
TSMFA

SEQ ID NO: 71 - protein, 5 aa, synthetic construct
TMFHA

SEQ ID NO: 72 - protein, 7 aa, synthetic construct
NTENNPD

SEQ ID NO: 73 - protein, 6 aa, synthetic construct
TENAPE

SEQ ID NO: 74 - protein, 4 aa, synthetic construct
EDGF

SEQ ID NO: 75 - protein, 8 aa, synthetic construct
GKWSQDLQ

SEQ ID NO: 76 - protein, 8 aa, synthetic construct
ILLANLHY

SEQ ID NO: 77 - protein, 7 aa, synthetic construct
RKRMSGT

SEQ ID NO: 78 - protein, 8 aa, synthetic construct
ILLLNVHM

SEQ ID NO: 79 - protein, 5 aa, synthetic construct
ENAPR

SEQ ID NO: 80 - protein, 5 aa, synthetic construct
THFFA

SEQ ID NO: 81 - protein, 5 aa, synthetic construct
DVSAC

SEQ ID NO: 82 - protein, 5 aa, synthetic construct
KASFA

SEQ ID NO: 83 - protein, 8 aa, synthetic construct
ILLHNVHM

SEQ ID NO: 84 - protein, 7 aa, synthetic construct
NTENSPE

SEQ ID NO: 85 - protein, 5 aa, synthetic construct
TQKEL

SEQ ID NO: 86 - protein, 6 aa, synthetic construct
GKWSQN

SEQ ID NO: 87 - protein, 5 aa, synthetic construct
LYSKE

SEQ ID NO: 88 - protein, 4 aa, synthetic construct
QDLK

SEQ ID NO: 89 - protein, 6 aa, synthetic construct
ILLSNV

SEQ ID NO: 90 - protein, 6 aa, synthetic construct
GKWSQQ

SEQ ID NO: 91 - protein, 7 aa, synthetic construct
NTENAPE

SEQ ID NO: 92 - protein, 6 aa, synthetic construct
TQNAPD

SEQ ID NO: 93 - protein, 5 aa, synthetic construct
THMFA

SEQ ID NO: 94 - protein, 7 aa, synthetic construct
QKRMSGT

SEQ ID NO: 95 - protein, 4 aa, synthetic construct
SIDS

SEQ ID NO: 96 - protein, 7 aa, synthetic construct
QTENNPE

SEQ ID NO: 97 - protein, 4 aa, synthetic construct
QNLQ

SEQ ID NO: 98 - protein, 5 aa, synthetic construct
THMFV

SEQ ID NO: 99 - protein, 4 aa, synthetic construct
HDLK

SEQ ID NO: 100 - protein, 5 aa, synthetic construct
SIDSF

SEQ ID NO: 101 - protein, 6 aa, synthetic construct
SHVRWV

SEQ ID NO: 102 - protein, 4 aa, synthetic construct
QDLT

SEQ ID NO: 103 - protein, 6 aa, synthetic construct
ILLHNV

SEQ ID NO: 104 - protein, 4 aa, synthetic construct
RKRM